(19) United States
(12) Patent Application Publication
(10) Pub. No.: US 2026/0116881 A1
(43) Pub. Date: Apr. 30, 2026

(54) KIF MODULATOR COMPOUNDS AND METHODS OF USE

(71) Applicant: Iambic Therapeutics, Inc., San Diego, CA (US)

(72) Inventors: Shawn Eric WRIGHT, San Diego, CA (US); Iriny BOTROUS, San Diego, CA (US); Joseph Michael DENNIS, San Diego, CA (US); Laurent GOMEZ, San Diego, CA (US); Svitlana KULYK, San Diego, CA (US); Anders Steen CHRISTENSEN, Copenhagen (DK); Stephen MUNOZ, Chula Vista, CA (US); Shane YOST, San Diego, CA (US); Zhongdong HUANG, Rancho Santa Fe, CA (US); Chao ZHANG, Oakland, CA (US); Alexander Buitrago SANTANILLA, San Diego, CA (US)

(21) Appl. No.: 19/354,621

(22) Filed: Oct. 9, 2025

Related U.S. Application Data
(60) Provisional application No. 63/706,408, filed on Oct. 11, 2024.
(60) Provisional application No. 63/721,953, filed on Nov. 18, 2024.
(60) Provisional application No. 63/736,512, filed on Dec. 19, 2024.
(60) Provisional application No. 63/765,353, filed on Feb. 28, 2025.
(60) Provisional application No. 63/784,691, filed on Apr. 7, 2025.
(60) Provisional application No. 63/789,634, filed on Apr. 16, 2025.

(51) Int. Cl. C07D 471/18; A61K 31/439; A61K 31/4545; A61K 31/496; A61K 31/4985; A61K 31/4995; A61K 31/5377; A61K 31/5386; A61K 31/541; A61K 31/695; A61P 35/00; C07B 59/00; C07D 498/18; C07D 519/00; C07F 7/08; C07K 14/47

(52) U.S. Cl. CPC C07D 471/18; A61K 31/439; A61K 31/4545; A61K 31/496; A61K 31/4985; A61K 31/4995; A61K 31/5377; A61K 31/5386; A61K 31/541; A61K 31/695; A61P 35/00; C07B 59/002; C07D 498/18; C07D 519/00; C07F 7/0816; C07K 14/47

(57) ABSTRACT

Provided herein are KIF modulator compounds, pharmaceutical compositions thereof, and methods of using said compounds and compositions in the treatment of disease.

### CROSS-REFERENCE TO RELATED APPLICATIONS

This application claims the benefit of U.S. Provisional Application No. 63/706,408, filed Oct. 11, 2024, U.S. Provisional Application No. 63/721,953, filed Nov. 18, 2024, U.S. Provisional Application No. 63/736,512, filed Dec. 19, 2024, U.S. Provisional Application No. 63/765,353, filed Feb. 28, 2025, U.S. Provisional Application No. 63/784,691, filed Apr. 7, 2025, and U.S. Provisional Application No. 63/789,634, filed Apr. 16, 2025, each of which is incorporated herein by reference in its entirety.

### BACKGROUND OF INVENTION

Cancers such as breast cancer, pancreatic cancer, uterine cancer, as well as other solid tumors, affect millions of patients. Cancer treatment represents a large component of the overall healthcare expenditure.

Kinesin family member 18A (KIF18A) has a distinct role in cell division as a member of the kinesin superfamily of microtubule-associated molecular motors. KIF18A is overexpressed in cancers such as breast, pancreatic, and uterine cancer, and has been shown to be associated with tumor aggressiveness.

### SUMMARY OF THE INVENTION

Chromosomal instability (CIN) is a hallmark of cancer that results from errors in chromosome segregation during mitosis. In some instances, targeting of CIN-associated vulnerabilities is a therapeutic strategy for treating cancer. In some instances, kinesin family member 18A (KIF18A) plays a role in maintaining bipolar spindle integrity, thereby promoting viability of CIN cancer cells. In some instances, cancer cells with chromosomal instability depend on KIF18A for cell division and survival, making KIF18A a potential therapeutic target.

Provided herein in some embodiments are KIF18A modulators (e.g., inhibitors). In some embodiments, the compounds provided herein are useful for treating cancer, such as cancers that depend on KIF18A for cell division and survival.

In one aspect, provided herein is a compound of Formula (I-1):

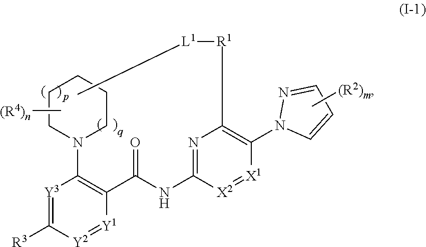

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), —CH(OR^{2a})-(optionally substituted phenyl), optionally substituted monocyclic or bicyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, optionally substituted 4- to 10-membered heterocycloalkyl, or optionally substituted monocyclic heteroaryl;
   or two R^{2a} on the same N atom are taken together with the N atom to which they are attached to form a 4- to 10-membered heterocycloalkyl which is optionally substituted with 1, 2, or 3 groups independently selected from halogen, —OH, oxo, —O(C₁-C₄ alkyl), —N(C₁-C₄ alkyl)₂, C₁-C₄ alkyl, C₁-C₄ haloalkyl, C₁-C₆ hydroxyalkyl —CN, —C(O)(C₁-C₆ alkyl), —S(O)₂(C₁-C₆ alkyl), and optionally substituted C₃-C₆ cycloalkyl;
   each R^{2b} is independently optionally substituted C₁-C₆ alkyl, optionally substituted C₃-C₁₀ cycloalkyl, or optionally substituted 4- to 10-membered heterocycloalkyl;
   each v is independently 1, 2, or 3;
R³ is —N(R^{3a})₂, —NR^{3a}(SO₂R^{3b}), —SO₂NR^{3a}R^{3b}, —SO(═NH)NR^{3a}R^{3b}, —NR^{3a}SO₂N(R^{3a})₂, —SO₂R^{3b}, —NR^{3a}C(O)—C(O)R^{3b}, —NR^{3a}C(O)OR^{3b}, or —NR^{3b}C(O)NR^{3a}R^{3a};
   each R^{3a} is independently hydrogen, optionally substituted C₁-C₆ alkyl, or C₁-C₆ hydroxyalkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR⁵, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4;
wherein if any L¹, L^{1b}, R², R^{2a}, R^{2b}, R³, R^{3a}, R^{3b}, R⁵, R^{5a}, R^{5b}, R⁶, R^{6a}, or R^{6b} group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl.

In some embodiments, provided herein is a compound of Formula (I-0):

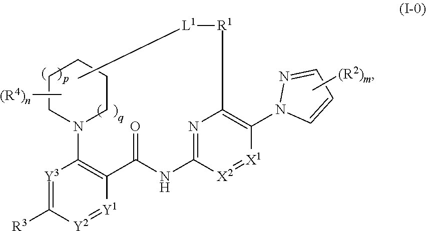

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R⁶, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
each R^{6a} is independently hydrogen or optionally substituted C₁-C₆ alkyl;
each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4;
wherein if any L¹, L^{1b}, R², R^{2a}, R^{2b}, R³, R^{3a}, R^{3b}, R⁵, R^{5a}, R^{5b}, R⁶, R^{6a}, or R^{6b} group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl.

In some embodiments, provided herein is a compound of Formula (I),

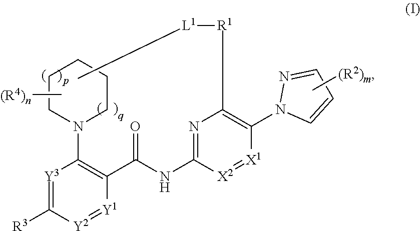

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹; L^{1a} is a bond, —O—, —NH—, —S—, or —SO₂—; L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6′}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4;
wherein if any L¹, L^{1b}, R², R^{2a}, R^{2b}, R³, R^{3a}, R^{3b}, R⁵, R^{5a}, R^{5b}, R⁶, R^{6a}, or R^{6b} group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl.

In some embodiments, the compound is a compound of Formula (II),

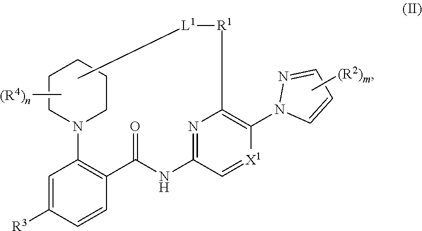

or a pharmaceutically acceptable salt, solvate, or isotopologue thereof, wherein:
X¹ is CH or N.

In some embodiments, the compound is a compound of Formula (IIa), Formula (IIb), or Formula (IIc).

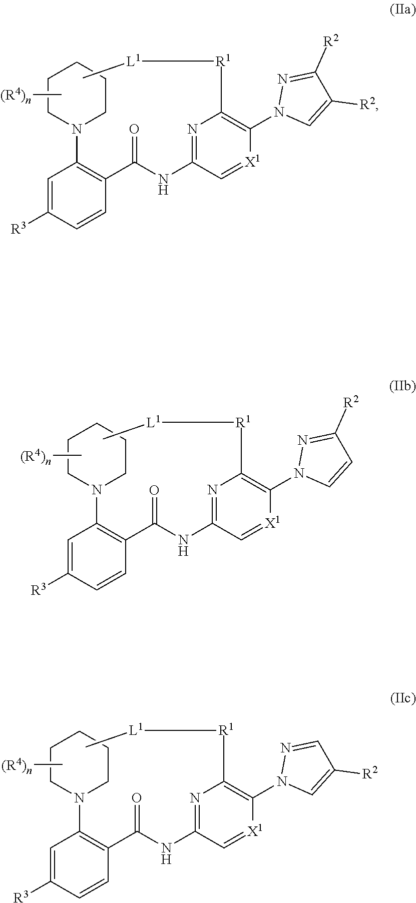

or a pharmaceutically acceptable salt, solvate, or isotopologue thereof, wherein:
X¹ is CH or N.

In some embodiments, the compound is a compound of Formula (IIIa), Formula (IIIb), Formula (IVa), or Formula (IVb),

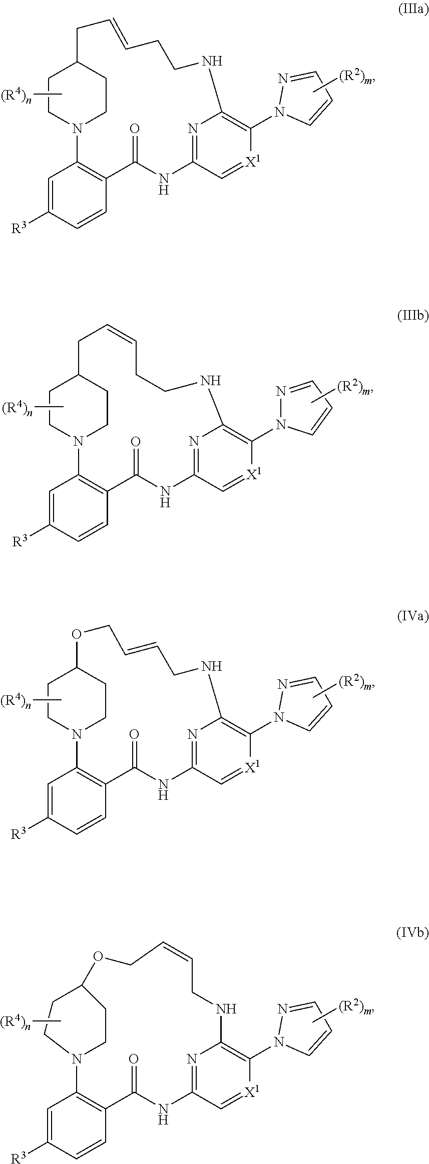

or a pharmaceutically acceptable salt, solvate, or isotopologue thereof, wherein:
X¹ is CH or N.

In some embodiments, the compound is a compound of Formula (VIa), Formula (VIb), Formula (VIIa), or Formula (VIIb),

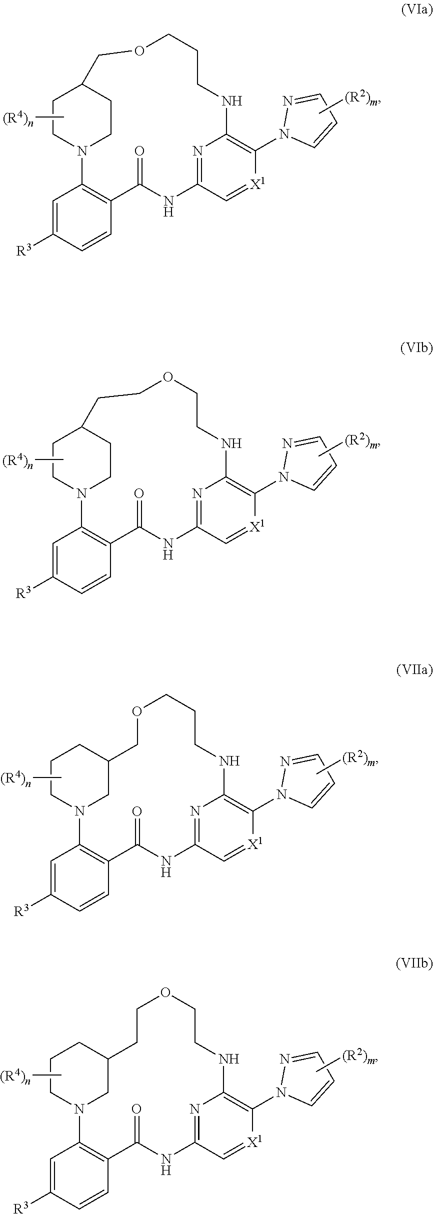

or a pharmaceutically acceptable salt, solvate, or isotopologue thereof, wherein:
X¹ is CH or N.

Any combination of the groups described above for the various variables is contemplated herein.

Also provided herein in some embodiments is a composition comprising a protein and a compound described herein, and the protein and the compound form the adduct described herein.

Also described herein is a pharmaceutical composition comprising a compound described herein, or a pharmaceutically acceptable salt, solvate, or isotopologue thereof, or the adduct thereof as described herein, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

Also described herein is a method of treating a disease or condition in a mammal that would benefit from the modulation of KIF18A activity comprising administering a compound described herein, or pharmaceutically acceptable salt, solvate, or isotopologue thereof, or the adduct thereof as described herein, to the mammal in need thereof. In some embodiments, the disease or condition is cancer. In some embodiments, the cancer is bladder cancer, endometrial cancer, lung squamous cell cancer, breast cancer, colon cancer, kidney cancer, liver cancer, lung cancer, small cell lung cancer, esophagus cancer, gall-bladder cancer, brain cancer, head and neck cancer, ovarian cancer, pancreas cancer, stomach cancer, cervix cancer, thyroid cancer, prostate cancer or skin cancer.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

For a more complete understanding of the features and advantages of the present disclosure, reference is now made to the detailed description of the disclosure along with the accompanying figures and in which:

FIG. 1 shows preclinical differentiation of A Compound of Formula (I) when compared to sovilnesib (also known as AMG650), a clinical stage KIF18A inhibitor compound under investigation for treatment of cancer. A Compound of Formula (I) is a highly potent KIF18A inhibitor with favorable in vitro activity and ADME-PK profile for drug development.

FIG. 2A shows a 6-day viability assay with A Compound of Formula (I) dose response curve in OVCAR-3 and MCF-7 cells. OVCAR-3 cells are sensitive to KIF18A inhibition by A Compound of Formula (I) and MCF-7 cells are not.

FIG. 2B shows a dose dependent increase in M phase quantified by flow cytometry using phospho-histone H3 (Ser10) Alexa 488 antibody+FxCycle™ Violet stain with A Compound of Formula (I).

FIG. 2C shows a dose response for 72 hours of OVCAR-3 (left panel) and MCF-7 (right panel) cells treated with A Compound of Formula (I). Incucyte v.2023A was used to quantify cell confluence and cell death (via Caspase-3/7 green apoptosis reagent). Treatment with A Compound of Formula (I) results in increased apoptosis and reduced confluence in OVCAR-3 cells but not MCF-7 cells.

FIG. 3 shows IC₅₀ data for A Compound of Formula (I) in a broad panel of cell lines, as assessed by 6-day CellTiter-Glo assay.

FIG. 4A shows that treatment with A Compound of Formula (I) at varying doses for 21 days achieves dose dependent antitumor activity.

FIG. 4B shows that treatment with A Compound of Formula (I) at varying doses for 21 days results in no body weight loss.

FIG. 4C shows the results of a study where mice bearing OVCAR-3 tumors were treated with A Compound of Formula (I) at 100 mg/kg QD PO×1 day, 2 days or 7 days. Tumors were harvested and stained for phospho-histone H3 (pHH3) and images were quantified for positive cells compared to total cells.

FIG. 4D shows the results of a study where mice bearing OVCAR-3 tumors were treated with A Compound of Formula (I) at 100 mg/kg QD PO×1 day, 2 days or 7 days. Tumors were harvested and stained for cleaved caspase-3 (CC-3) and images were quantified for positive cells compared to total cells.

FIG. 4E shows that A Compound of Formula (I) achieves similar antitumor activity as AMG650, but at ˜10× lower total (left panel) and unbound (right panel) drug exposures.

FIG. 5A shows that treatment with A Compound of Formula (I) led to significant tumor regression at 100, 200 and 400 mg/kg dose levels in MFM-223 cells (top panel); while AMG650 treatment led to significant tumor regression at 100 mg/kg however the study had to be stopped on day 9 at dose levels >100 mg/kg (bottom panel).

FIG. 5B shows that all dose levels of A Compound of Formula (I) were well tolerated throughout the treatment duration; while AMG650 was not tolerated at doses above 100 mg/kg QD.

FIG. 6A shows that A Compound of Formula (I) results in dose dependent antitumor activity in a OVCAR-8 xenograft study. A Compound of Formula (I) at 100 and 250 mg/kg QD and AMG650 at 100 mg/kg QD result in similar regression in OVCAR-8 xenograft.

FIG. 6B shows that plasma and tumor PK are correlated to depth of tumor growth inhibition (TGI) response. Similar efficacy is achieved with A Compound of Formula (I) as compared to AMG650 with ˜10× less plasma and tumor exposure.

FIG. 7 shows that A Compound of Formula (I) at 100 mg/kg QD×˜21 days results in anti-tumor activity across a panel of breast and ovarian cell line derived xenograft models. A Compound of Formula (I) achieved regressions in 3/7 (43%) of breast and 2/4 (50%) of ovarian cancer models tested.

### DETAILED DESCRIPTION OF THE INVENTION

The KIF18A gene is part of the Kinesin-8 subfamily. KIF18A is a plus-end-directed motor. In some instances, KIF18A influences dynamics at the plus end of kinetochore microtubules, such as controlling chromosomal positioning and spindle tension. In some instances, depletion of KIF18A leads to longer spindles, increased chromosome oscillation at metaphase, and activation of the mitotic spindle assembly checkpoint.

KIF18A is a potential target for treating cancer, such as cancers where KIF18A is overexpressed and/or that depend on KIF18A for cell division and survival. For example, such cancers include colon, breast, lung, pancreas, prostate, bladder, head, neck, cervix, and ovarian cancers. In some instances, genetic deletion, knockdown, or inhibition of KIF18A affects mitotic spindle apparatus. In some instances, inhibition of KIF18A induces mitotic cell arrest. In some instances, mitotic cell arrest is a vulnerability that can promote cell death in mitosis via apoptosis, mitotic catastrophe, or multipolarity driven lethality or death after mitotic slippage in interphase.

Provided in some embodiments herein are KIF18A modulators. In some embodiments, the compounds provided herein are useful for treating cancer, such as cancers that depend on KIF18A for cell division and survival.

### Compounds

The compounds described herein can be useful as KIF18A modulators. In some embodiments, the compounds described herein are KIF18A inhibitors.

In one aspect, provided herein is a compound of Formula (I-1):

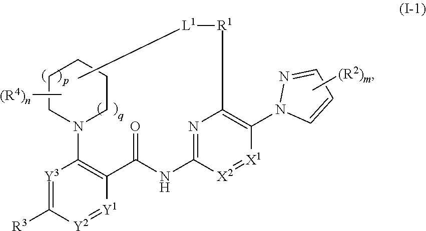

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), —CH(OR^{2a})-(optionally substituted phenyl), optionally substituted monocyclic or bicyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, optionally substituted 4- to 10-membered heterocycloalkyl, or optionally substituted monocyclic heteroaryl;
   or two R^{2a} on the same N atom are taken together with the N atom to which they are attached to form a 4- to 10-membered heterocycloalkyl which is optionally substituted with 1, 2, or 3 groups independently selected from halogen, —OH, oxo, —O(C₁-C₄ alkyl), —N(C₁-C₄ alkyl)₂, C₁-C₄ alkyl, C₁-C₄ haloalkyl, C₁-C₆ hydroxyalkyl —CN, —C(O)(C₁-C₆ alkyl), —S(O)₂(C₁-C₆ alkyl), and optionally substituted C₃-C₆ cycloalkyl;
   each R^{2b} is independently optionally substituted C₁-C₆ alkyl, optionally substituted C₃-C₁₀ cycloalkyl, or optionally substituted 4- to 10-membered heterocycloalkyl;
   each v is independently 1, 2, or 3;
R³ is —N(R^{3a})₂, —NR^{3a}(SO₂R^{3b}), —SO₂NR^{3a}R^{3b}, —SO(═NH)NR^{3a}R^{3b}, —NR^{3a}SO₂N(R^{3a})₂, —SO₂R^{3b}, —NR^{3a}C(O)—C(O)R^{3b}, —NR^{3a}C(O)OR^{3b}, or —NR^{3b}C(O)NR^{3a}R^{3a};
   each R^{3a} is independently hydrogen, optionally substituted C₁-C₆ alkyl, or C₁-C₆ hydroxyalkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.

In some embodiments,
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3; and
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or;
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl.

In some embodiments, described herein, is a compound of Formula (I-0):

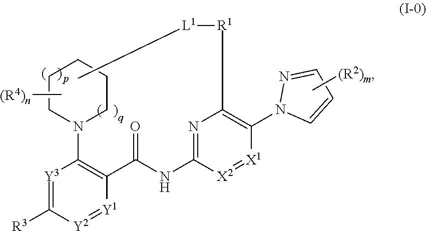

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.

In some embodiments, described herein is a compound having the structure of Formula (I):

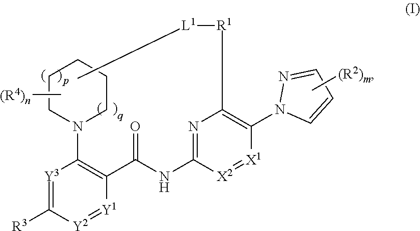

or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR⁵, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.

In some embodiments of any of the formulas described herein, if any L¹, L^{1b}, R², R^{2a}, R^{2b}, R³, R^{3a}, R^{3b}, R⁵, R^{5a}, R^{5b}, R⁶, R^{6a}, or R^{6b} group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl.

For any and all of the embodiments, substituents are selected from among a subset of the listed alternatives. For example, in some embodiments, L¹ is a linker comprising the structure -L^{1a}-L^{1b}-*, where * designates the attachment point to R¹; L^{1a} is a bond, —O—, —NH—, —S—, or —SO₂—; L^{1b} is optionally substituted C₄-C₅ alkenylene. In other embodiments, L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.

In some embodiments of Formula (I), or a pharmaceutically acceptable salt, solvate, or isotopologue thereof,
Y, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b}; each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.

In some embodiments,
each R² is independently halogen, —CN, —OR^{2a}, —N(R^{2a})₂, —C(O)N(R^{2a})₂, —N(R^{2a})C(O)NR^{2a}, —C(O)R^{2b}, —C(O)OR^{2a}, —S(O)₂N(R^{2a})₂, —S(O)₂R^{2b}, optionally substituted C₁-C₈ alkyl, C₁-C₈ hydroxyalkyl, optionally substituted C₃-C₈ cycloalkyl, or optionally substituted 4- to 8-membered heterocycloalkyl;
   each R^{2a} is independently hydrogen, optionally substituted C₁-C₆ alkyl, or optionally substituted C₃-C₆ cycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl; and
   each R^{2b} is independently optionally substituted C₁-C₆ alkyl.

In some embodiments of Formula (Ib), or a pharmaceutically acceptable salt, solvate, or isotopologue thereof,
Y¹, Y², and Y³ are each independently CR⁵ or N;
X¹ and X² are each independently CR⁶ or N;
L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁—C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.

In some embodiments,
each R² is independently halogen, —CN, —OR^{2a}, —N(R^{2a})₂, —C(O)N(R^{2a})₂, —N(R^{2a})C(O)NR^{2a}, —C(O)R^{2b}, —C(O)OR^{2a}, —S(O)₂N(R^{2a})₂, —S(O)₂R^{2b}, optionally substituted C₁-C₈ alkyl, C₁-C₈ hydroxyalkyl, optionally substituted C₃-C₈ cycloalkyl, or optionally substituted 4- to 8-membered heterocycloalkyl;
   each R^{2a} is independently hydrogen, optionally substituted C₁-C₆ alkyl, or optionally substituted C₃-C₆ cycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl; and
   each R^{2b} is independently optionally substituted C₁-C₆ alkyl.

In some embodiments, Y¹ is CR⁵. In other embodiments, Y¹ is N.

In some embodiments, Y² is CR⁵. In other embodiments, Y² is N.

In some embodiments, Y³ is CR⁵. In other embodiments, Y³ is N.

In some embodiments, Y¹ is CR⁵, Y² is CR⁵, and Y³ is CR⁵.

In some embodiments, each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl; each R^{5a} is independently hydrogen or optionally substituted C₁-C₆ alkyl; and each R^{5b} is independently optionally substituted C₁-C₆ alkyl. In some embodiments, each R⁵ is independently hydrogen, halogen, —CN, —OR^{5a}, —N(R^{5a})₂, or C₁-C₈ alkyl. In some embodiments, each R⁵ is independently hydrogen, halogen, —CN, —OR^{5a}, —N(R^{5a})₂, or optionally substituted C₁-C₈ alkyl; each R^{5a} is independently hydrogen or C₁-C₆ alkyl; and each R^{5b} is independently C₁-C₆ alkyl. In some embodiments, each R⁵ is independently hydrogen, halogen, —CN, —OH, —O(C₁-C₆ alkyl), —NH₂, —NH(C₁-C₆ alkyl), —N(C₁-C₆ alkyl)₂, or C₁-C₈ alkyl. In some embodiments, each R⁵ is independently hydrogen, halogen, or C₁-C₆ alkyl. In some embodiments, each R⁵ is independently hydrogen, fluoro, chloro, or methyl. In some embodiments, each R⁵ is hydrogen or fluoro. In some embodiments, each R⁵ is hydrogen.

In some embodiments, Y¹ is CR⁵; Y² is CR⁵; Y³ is CR⁵; and each R⁵ is independently hydrogen, halogen, —CN, —OR^{5a}, —N(R^{5a})₂, or C₁-C₈ alkyl. In some embodiments, each R⁵ is hydrogen or fluoro. In some embodiments, each R⁵ is hydrogen.

In some embodiments, Y¹ is CH; Y² is CH; and Y³ is CH.

In some embodiments, X¹ is CR⁶. In other embodiments, X¹ is N.

In some embodiments, X² is CR⁶. In other embodiments, X² is N.

In some embodiments, X¹ is CR⁶ or N; and X² is CR⁶.

In some embodiments, each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl; each R^{6a} is independently hydrogen or optionally substituted C₁-C₆ alkyl; and each R^{6b} is independently optionally substituted C₁-C₆ alkyl. In some embodiments, each R⁶ is independently hydrogen, halogen, —CN, —OR^{6a}, —N(R^{6a})₂, or C₁-C₈ alkyl. In some embodiments, each R⁶ is independently hydrogen, halogen, —CN, —OR^{6a}, —N(R^{6a})₂, or optionally substituted C₁-C₈ alkyl; each R^{6a} is independently hydrogen or C₁-C₆ alkyl; and each R^{6b} is independently C₁-C₆ alkyl. In some embodiments, each R⁶ is independently hydrogen, halogen, —CN, —OH, —O(C₁-C₆ alkyl), —NH₂, —NH(C₁-C₆ alkyl), —N(C₁-C₆ alkyl)₂, or C₁-C₈ alkyl. In some embodiments, each R⁶ is independently hydrogen, halogen, or C₁-C₆ alkyl. In some embodiments, each R⁶ is independently hydrogen, fluoro, chloro, or methyl. In some embodiments, each R⁶ is hydrogen or fluoro. In some embodiments, each R⁶ is hydrogen.

In some embodiments, X¹ is CR⁶ or N; X² is CR⁶; and each R⁶ is independently hydrogen, halogen, —CN, —OR^{5a}, —N(R^{5a})₂, or C₁-C₈ alkyl. In some embodiments, each R⁶ is hydrogen or fluoro. In some embodiments, each R⁶ is hydrogen.

In some embodiments, X¹ is CH or N; and Y² is CH. In some embodiments, X¹ is CH; and Y² is CH. In some embodiments, X¹ is N; and Y² is CH.

In some embodiments, p is 0, 1, or 2. In some embodiments, p is 0. In some embodiments, p is 1. In some embodiments, p is 2.

In some embodiments, q is 0, 1, or 2. In some embodiments, q is 0. In some embodiments, q is 1. In some embodiments, q is 2.

In some embodiments, p is 1; and q is 1.

In some embodiments, the compound is a compound of Formula (II),

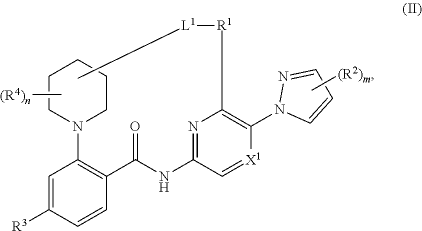

or a pharmaceutically acceptable salt, solvate, or isotopologue thereof, wherein X¹ is CH or N. In some embodiments X¹ is CH. In some embodiments, X¹ is N.

In some embodiments, the compound is a compound of Formula (IIa), Formula (IIb), or Formula (IIc).

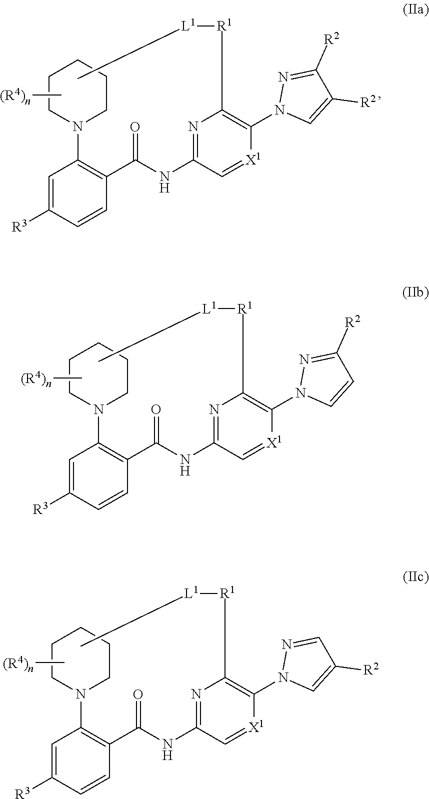

or a pharmaceutically acceptable salt, solvate, or isotopologue thereof, wherein:
X¹ is CH or N.

In some embodiments, R¹ is a bond, —O—, —NH—, or —N(C₁-C₄ alkyl)-. In some embodiments, R¹ is a bond. In some embodiments, R¹ is —O—, —NH—, or —N(C₁-C₄ alkyl)-. In some embodiments, R¹ is —NH—, or —N(C₁-C₄ alkyl)-. In some embodiments, R¹ is —O—. In some embodiments, R¹ is —NH—. In some embodiments, R¹ is —N(C₁-C₄ alkyl)-. In some embodiments R¹ is —NH— or —N(CH₃)—. In some embodiments, R¹ is —N(CH₃)—.

In some embodiments, L¹ is a linker comprising the structure -L^{1a}-L^{1b}-*, where * designates the attachment point to R¹. In some embodiments, L¹ can be any suitable linker structure in any combination of variables L^{1a} and Lib described herein.

In some embodiments, L¹ is attached to the non-aromatic heterocyclic ring (e.g., the piperidine ring) at the 3-position of the piperidine ring. In other embodiments, L¹ is attached to the non-aromatic heterocyclic ring (e.g., the piperidine ring) at the 4-position of the piperidine ring.

In some embodiments, of Formula (I), is

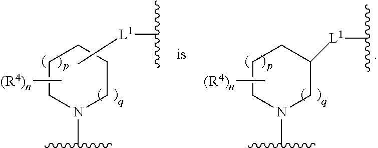

In other embodiments of Formula (I),

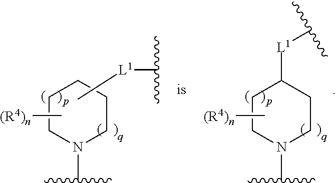

In some embodiments, of Formula (II), is

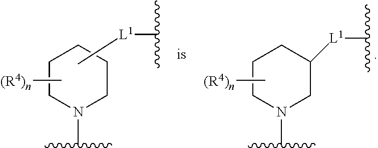

In other embodiments of Formula (II),

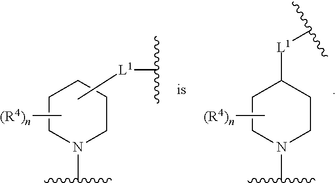

In some embodiments, L¹ is a linker comprising the structure -L^{1a}-L^{1b}-*, where * designates the attachment point to R¹; L^{1a} is a bond, —O—, —NH—, —S—, or —SO₂—; and L^{1b} is optionally substituted C₄-C₅ alkenylene.

In some embodiments, L^{1a} is a bond, —O—, —NH—, —S—, or —SO₂—. In some embodiments, L^{1a} is a bond or —O—. In some embodiments, L^{1a} is a bond. In some embodiments, L^{1a} is —O—. In some embodiments, L^{1a} is —NH—. In some embodiments, L^{1a} is —S—. In some embodiments, L^{1a} is —SO₂—.

In some embodiments, L^{1b} is optionally substituted C₄-C₅ alkenylene. In some embodiments, L^{1b} is unsubstituted C₄-C₅ alkenylene. In some embodiments, L^{1b} is C₄-C₅ alkenylene. In some embodiments, L^{1b} is optionally substituted C₄ alkenylene. In some embodiments, L^{1b} is unsubstituted C₄ alkenylene. In some embodiments, L^{1b} is C₄ alkenylene. In some embodiments, L^{1b} is optionally substituted C₅ alkenylene. In some embodiments, L^{1b} is unsubstituted C₅ alkenylene. In some embodiments, L^{1b} is C₅ alkenylene.

In some embodiments, L^{1b} is —CH═CHCH₂CH₂CH₂—*, —CH₂CH═CHCH₂CH₂—*, —CH₂CH₂CH═CHCH₂—*, —CH₂CH₂CH₂CH═CH—*, —CH═CHCH₂CH₂—*, —CH₂CH═CHCH₂—*, or —CH₂CH₂CH═CH—*, where * designates the attachment point to RU.

In some embodiments, L bis an (E)-alkenylene. In other embodiments, L^{1b} is a (Z)-alkenylene. In some embodiments, L^{1b} is

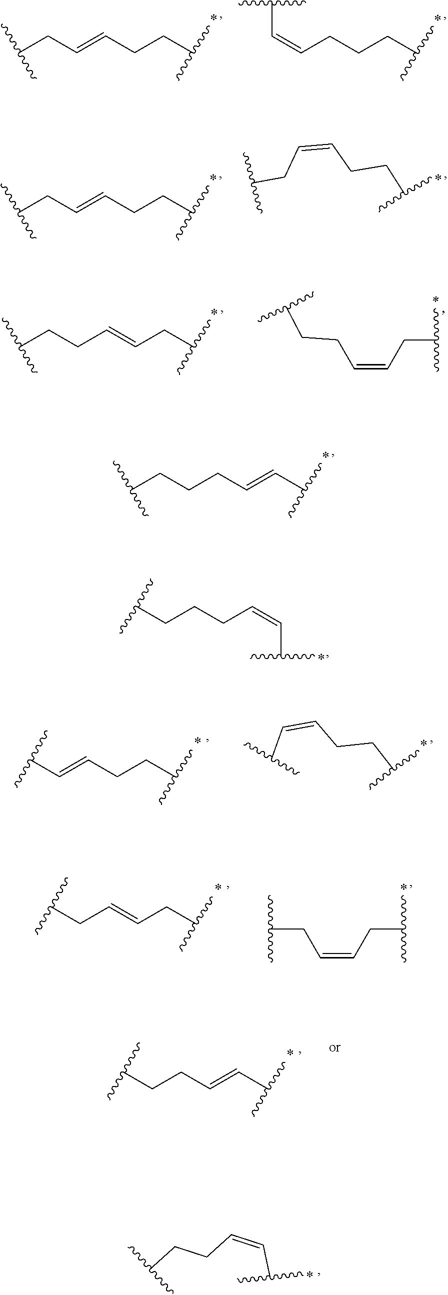

where * designates the attachment point to RU.

In some embodiments, L^{1a} is a bond; and L^{1b} is C₅ alkenylene.

In some embodiments, L^{1a} is —O—; and L^{1b} is C₄ alkenylene.

In some embodiments, L^{1a} is a bond; and L^{1b} is C₅ alkenylene; or L^{1a} is —O—; and L^{1b} is C₄ alkenylene.

In some embodiments, L¹ is —CH═CHCH₂CH₂CH₂—*, —CH₂CH═CHCH₂CH₂—*, —CH₂CH₂CH═CHCH₂—*, —CH₂CH₂CH₂CH═CH—*, —OCH═CHCH₂CH₂—*, —OCH₂CH═CHCH₂—*, or —OCH₂CH₂CH═CH—*, where * designates the attachment point to RU. In some embodiments, L¹ is —CH₂CH═CHCH₂CH₂—*, —CH₂CH₂CH═CHCH₂—*, or —OCH₂CH═CHCH₂—*, where * designates the attachment point to RU. In some embodiments, L¹ is —CH₂CH═CHCH₂CH₂—* or —OCH₂CH═CHCH₂—*, where * designates the attachment point to R¹.

In some embodiments, L¹ is

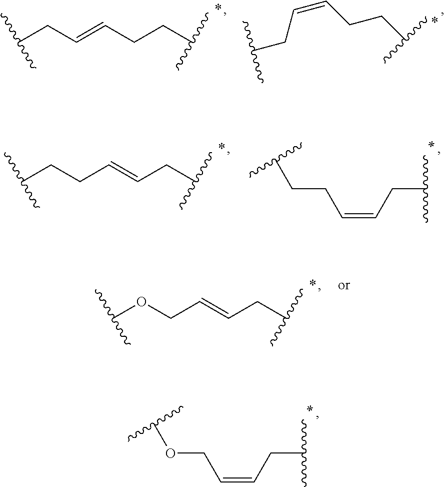 [SMILES: *C/C=C/CC* ; *C/C=C\CC* ; *C/C=C/CC* ; *C/C=C\CC* ; *C/C=C/CO* ; *C/C=C\CO*]

where * designates the attachment point to R¹.

In some embodiments, L¹ is

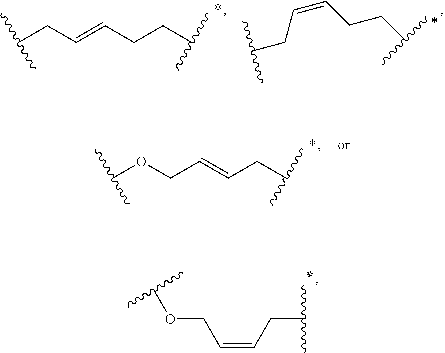 [SMILES: *C/C=C/CC* ; *C/C=C\CC* ; *C/C=C/CO* ; *C/C=C\CO*]

where * designates the attachment point to R¹.

In some embodiments, the compound is a compound of Formula (IIIa) or Formula (IIIb),

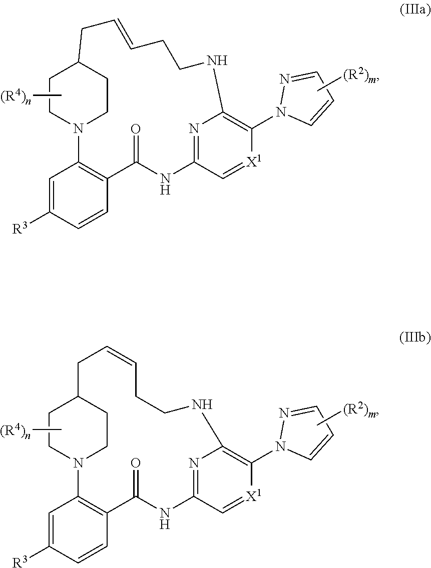

or a pharmaceutically acceptable salt, solvate, or isotopologue thereof, wherein X¹ is CH or N. In some embodiments X¹ is CH. In some embodiments, X¹ is N.

In some embodiments, the compound is a compound of Formula (IIIa), or a pharmaceutically acceptable salt, solvate, or isotopologue thereof. In some embodiments, the compound is a compound of Formula (IIIb), or a pharmaceutically acceptable salt, solvate, or isotopologue thereof.

In some embodiments, the compound is a compound of Formula (IVa) or Formula (IVb),

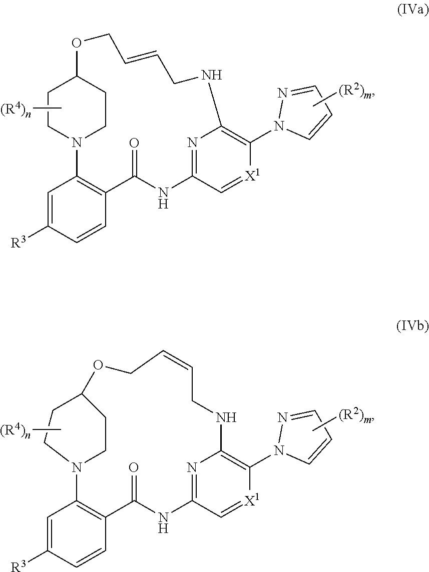

or a pharmaceutically acceptable salt, solvate, or isotopologue thereof, wherein X¹ is CH or N. In some embodiments X¹ is CH. In some embodiments, X¹ is N.

In some embodiments, the compound is a compound of Formula (IVa), or a pharmaceutically acceptable salt, solvate, or isotopologue thereof. In some embodiments, the compound is a compound of Formula (IVb), or a pharmaceutically acceptable salt, solvate, or isotopologue thereof.

In some embodiments, the compound is a compound of Formula (Va) or Formula (Vb),

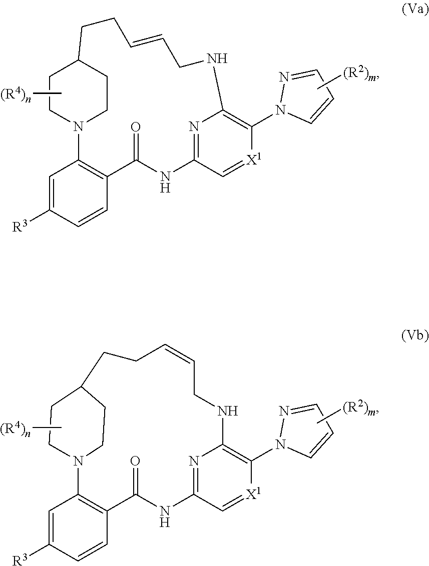

or a pharmaceutically acceptable salt, solvate, or isotopologue thereof, wherein X¹ is CH or N. In some embodiments X¹ is CH. In some embodiments, X¹ is N.

In some embodiments, the compound is a compound of Formula (Va), or a pharmaceutically acceptable salt, solvate, or isotopologue thereof. In some embodiments, the compound is a compound of Formula (Vb), or a pharmaceutically acceptable salt, solvate, or isotopologue thereof.

In some embodiments, L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S. In some embodiments, the heteroalkylene is branched. In other embodiments, the heteroalkylene is a straight-chained heteroalkylene.

In some embodiments, L¹ is optionally substituted C₄-C₅ heteroalkylene; wherein the terminal atoms of the heteroalkylene are C, and the heteroalkylene comprises 1 heteroatom selected from N, O, and S. In some embodiments, L¹ is optionally substituted C₄ heteroalkylene; wherein the terminal atoms of the heteroalkylene are C, and the heteroalkylene comprises 1 heteroatom selected from N, O, and S. In some embodiments, L¹ is optionally substituted C₅ heteroalkylene; wherein the terminal atoms of the heteroalkylene are C, and the heteroalkylene comprises 1 heteroatom selected from N, O, and S. In some embodiments, L¹ is optionally substituted C₆ heteroalkylene; wherein the terminal atoms of the heteroalkylene are C, and the heteroalkylene comprises 1 heteroatom selected from N, O, and S.

In some embodiments, L¹ is optionally substituted C₄-C₅ heteroalkylene; wherein the terminal atoms of the heteroalkylene are C, and the heteroalkylene comprises 1 O atom. In some embodiments, L¹ is optionally substituted C₄ heteroalkylene; wherein the terminal atoms of the heteroalkylene are C, and the heteroalkylene comprises 1 O atom. In some embodiments, L¹ is optionally substituted C₅ heteroalkylene; wherein the terminal atoms of the heteroalkylene are C, and the heteroalkylene comprises 1 O atom. In some embodiments, L¹ is optionally substituted C₆ heteroalkylene; wherein the terminal atoms of the heteroalkylene are C, and the heteroalkylene comprises 1 O atom.

In some embodiments, L¹ is —CH₂OCH₂CH₂CH₂—* or —CH₂CH₂OCH₂CH₂—*, where * designates the attachment point to R¹.

In some embodiments, the compound is a compound of Formula (VIa), Formula (VIb), Formula (VIIa), or Formula (VIIb),

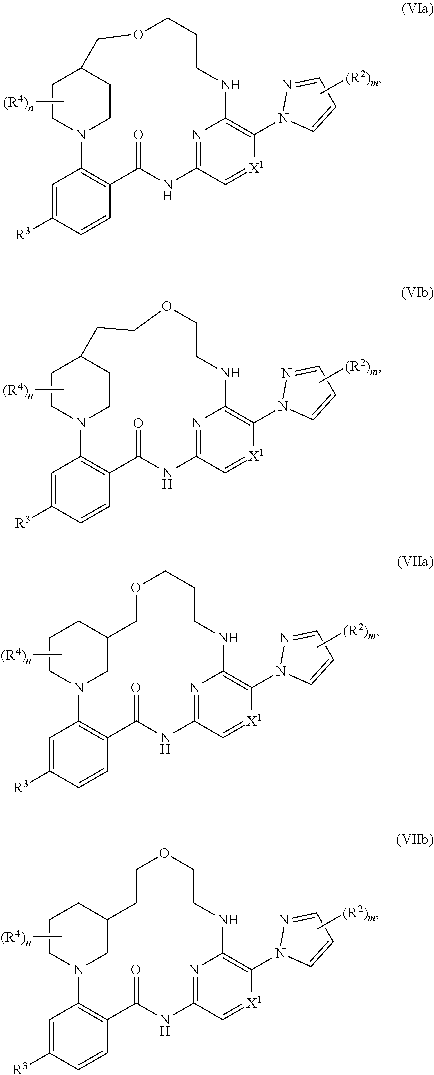

or a pharmaceutically acceptable salt, solvate, or isotopologue thereof, wherein X¹ is CH or N. In some embodiments X¹ is CH. In some embodiments, X¹ is N.

In some embodiments, the compound is a compound of Formula (VIa), or a pharmaceutically acceptable salt, solvate, or isotopologue thereof. In some embodiments, the compound is a compound of Formula (VIb), or a pharmaceutically acceptable salt, solvate, or isotopologue thereof. In some embodiments, the compound is a compound of Formula (VIIa), or a pharmaceutically acceptable salt, solvate, or isotopologue thereof. In some embodiments, the compound is a compound of Formula (VIIb), or a pharmaceutically acceptable salt, solvate, or isotopologue thereof.

In some embodiments, each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), —CH(OR^{2a})-(optionally substituted phenyl), optionally substituted monocyclic or bicyclic heteroaryl, or —(CH₂)ᵥ-(optionally substituted monocyclic heteroaryl); or two R² are joined together to form an optionally substituted C₃-C₁₀ cycloalkyl or an optionally substituted 4- to 10-membered heterocycloalkyl; each R^{2a} is independently hydrogen, optionally substituted C₁-C₆ alkyl, optionally substituted C₃-C₁₀ cycloalkyl, optionally substituted 4- to 10-membered heterocycloalkyl, or optionally substituted monocyclic heteroaryl; or two R^{2a} on the same N atom are taken together with the N atom to which they are attached to form a 4- to 10-membered heterocycloalkyl which is optionally substituted with 1, 2, or 3 groups independently selected from halogen, —OH, oxo, —O(C₁-C₄ alkyl), —N(C₁-C₄ alkyl)₂, C₁-C₄ alkyl, C₁-C₄ haloalkyl, C₁-C₆ hydroxyalkyl —CN, —C(O)(C₁-C₆ alkyl), —S(O)₂(C₁-C₆ alkyl), and optionally substituted C₃-C₆ cycloalkyl; each R^{2b} is independently optionally substituted C₁-C₆ alkyl, optionally substituted C₃-C₁₀ cycloalkyl, or optionally substituted 4- to 10-membered heterocycloalkyl; each v is independently 1, 2, or 3.

In some embodiments, each R² is independently halogen, —CN, —CH₂CH₂CN, —OR^{2a}, —O—CH₂—R^{2a}, —O—(CH₂)₂—R^{2a}, —N(R^{2a})₂, —CH₂—N(R^{2a})₂, —C(O)N(R^{2a})₂, —N(R^{2a})C(O)NR^{2a}, —C(O)(C₁-C₆ alkyl), —S(O)₂N(R^{2a})₂, —S(O)₂R^{2b}, —CH₂—S(O)₂R^{2b}, C₁-C₈ alkyl, C₁-C₈ hydroxyalkyl, optionally substituted C₃-C₆ cycloalkyl, —CH₂-(optionally substituted C₃-C₆ cycloalkyl), heteroaryl, optionally substituted 4- to 8-membered heterocycloalkyl, —CH₂-(optionally substituted 4- to 10-membered heterocycloalkyl), —CH(OR^{2a})-(phenyl), optionally substituted monocyclic or bicyclic heteroaryl; each R^{2a} is independently hydrogen, C₁-C₆ alkyl, C₃-C₆ cycloalkyl, 4- to 9-membered heterocycloalkyl, or monocyclic heteroaryl; wherein the alkyl, the C₃-C₆ cycloalkyl, and the 4- to 9-membered heterocycloalkyl is optionally substituted with 1, 2, or 3 groups independently selected from C₁-C₂ alkyl, halogen, —N(C₁-C₂ alkyl)₂, —OH, —CN, —C(O)(C₁-C₂ alkyl), —S(O)₂(C₁-C₂ alkyl), 4- to 6-membered heterocycloalkyl, C₃-C₆ cycloalkyl, and heteroaryl; or two R^{2a} on the same N atom are taken together with the N atom to which they are attached to form a 4- to 9-membered heterocycloalkyl which is optionally substituted with 1, 2, or 3 groups independently selected from halogen, —OH, oxo, —O(C₁-C₂ alkyl), —N(C₁-C₂ alkyl)₂, C₁-C₂ alkyl, C₁-C₂ haloalkyl, C₁-C₃ hydroxyalkyl —CN, —C(O)(C₁-C₂ alkyl), —S(O)₂(C₁-C₂ alkyl), and C₃-C₆ cycloalkyl, wherein the C₃-C₆ cycloalkyl is optionally substituted with 1, 2, or 3 groups independently selected from —OH; or two R^{2a} on the same N atom are taken together with the N atom to which they are attached to form a 4- to 6-membered heterocycloalkyl which is optionally substituted with 2 to 10 deuterium atoms; and each R^{2b} is independently C₁-C₆ alkyl, C₃-C₁₀ cycloalkyl, or 4- to 10-membered heterocycloalkyl.

In some embodiments, each R² is independently —F, —C₁, —Br, —CN, —CF₃, —OR^{2a}, —N(R^{2a})₂, —C(O)(C₁-C₂ alkyl), —C(O)N(R^{2a})₂, —S(O)₂R^{2b}, S(O)₂N(R^{2a})₂, C₁-C₄ hydroxyalkyl, —CH₂CF₃, —CH₂CH₂CN, —CH₂N(CH₃)₂, —CH₂S(O)₂CH₃, —CH₂NHC(O)CH₃

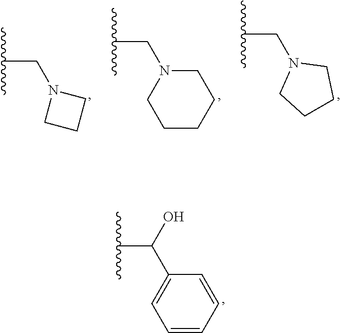 [SMILES: *CN1CCC1 ; *CN1CCCCC1 ; *CN1CCCC1 ; *C(O)c1ccccc1]

N(R^{2a})C(O)R^{2b},

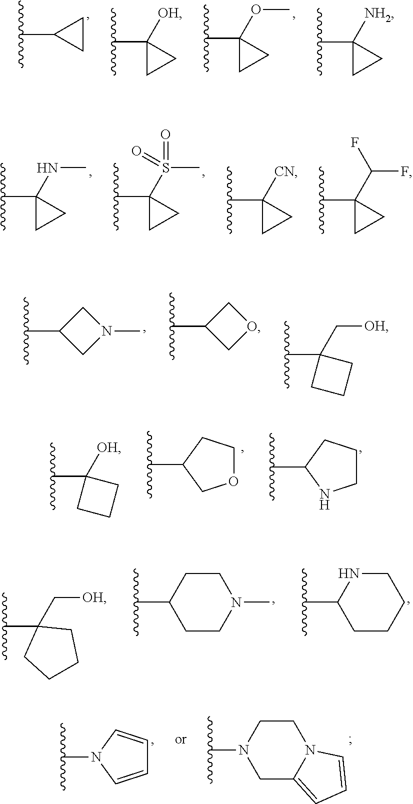

each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, —CH(CH₃)CH₂OH, —C(CH₃)₂CH₂OH,

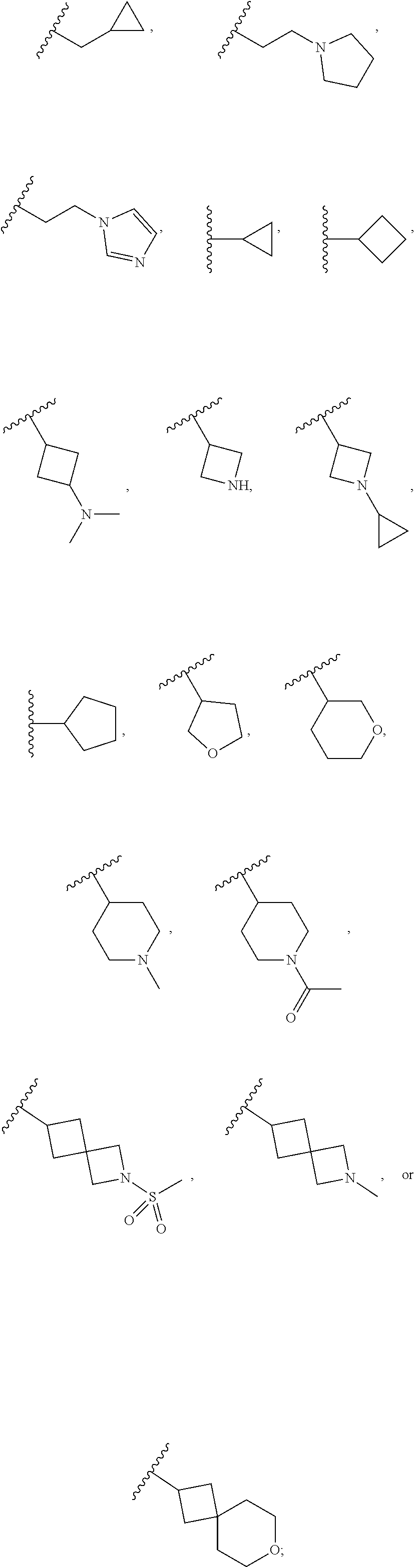

or two R^{2a} on the same N atom are taken together with the N atom to which they are attached to form a 4- to 9-membered heterocycloalkyl selected from an azetidinyl, pyrrolidinyl, piperidinyl, morpholinyl, piperazinyl, 3-oxa-8-azabicyclo[3.2.1]octanyl, 2-oxa-5-azabicyclo[2.2.1]heptanyl, oxazolidinyl, 4-oxa-7-azaspiro[2.5]octanyl, 2-oxa-6-azaspiro[3.3]heptanyl, 2-thia-6-azaspiro[3.4]octanyl, 2,7-diazaspiro[4.4]nonanyl, 2-oxa-7-azaspiro[4.4]nonanyl, 1-oxa-6-azaspiro[3.4]octanyl, 2,6-diazaspiro[3.4]octanyl, 2-thia-6-azaspiro[3.4]octanyl, thiomorpholinyl, isothiazolidinyl, and 1,4-azasilinanyl, wherein the heterocycloalkyl is optionally substituted with 1, 2, or 3 groups independently selected from —F, —OH, —OCH₃, oxo, —CH₃, —CH₂F, —CHF₂, —CF₃, —CN, —N(CH₃)₂, —S(O)₂CH₃,

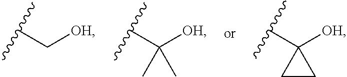

and C(O)CH₃; or two R^{2a} on the same N atom are taken together with the N atom to which they are attached to form a 5- to 6-membered heterocycloalkyl selected from a pyrrolidinyl and a piperidinyl, wherein the pyrrolidinyl and the piperidinyl is substituted with 2-10 deuterium atoms; and each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, or

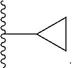 [SMILES: *C1CC1]

In some embodiments, each R² is independently —F, —CF₃, —Br, —Cl, —CN, —OH, —OCH₃, —C(O)CH₂CH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂CF₃, —CH₂CH₂CN, —CH₂OH, —CH₂N(CH₃)₂, —CH₂S(O)₂CH₃, —CH₂NHC(O)CH₃,

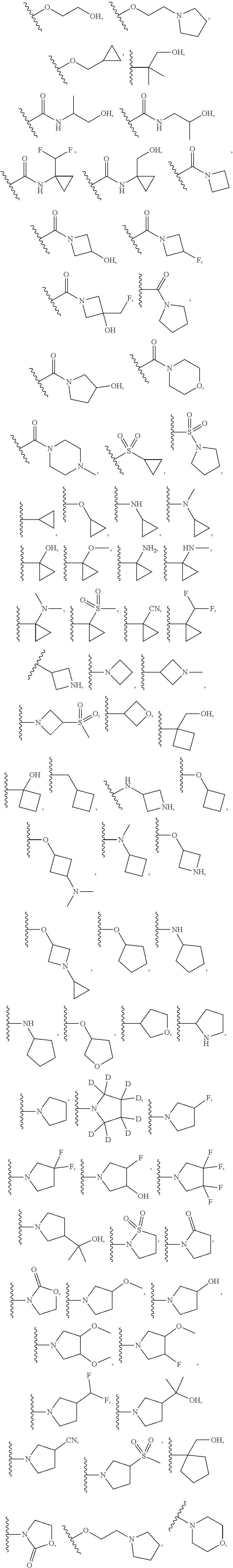

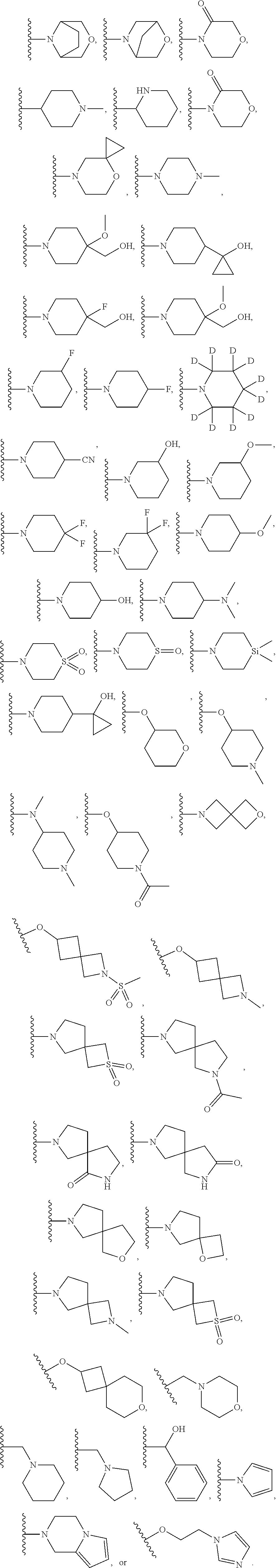

In some embodiments, each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or two R² are joined together to form an optionally substituted C₃-C₁₀ cycloalkyl or an optionally substituted 4- to 10-membered heterocycloalkyl; each R^{2a} is independently hydrogen, optionally substituted C₁-C₆ alkyl, optionally substituted C₃-C₁₀ cycloalkyl, or optionally substituted 4- to 10-membered heterocycloalkyl; or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; each v is independently 1, 2, or 3

In some embodiments, each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂—(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, —(CH₂)ᵥ-(optionally substituted monocyclic heteroaryl), or two R^{2b} are joined together to form an optionally substituted C₃-C₁₀ cycloalkyl or an optionally substituted 4- to 10-membered heterocycloalkyl; each R^{2a} is independently hydrogen, optionally substituted C₁-C₆ alkyl, optionally substituted C₃-C₁₀ cycloalkyl, or optionally substituted 4- to 10-membered heterocycloalkyl; or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); each R^{2a} is independently hydrogen, optionally substituted C₁-C₆ alkyl, optionally substituted C₃-C₁₀ cycloalkyl, or optionally substituted 4- to 10-membered heterocycloalkyl; or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₈ cycloalkyl, —(CH₂)ᵥ-(optionally substituted C₃-C₈ cycloalkyl), optionally substituted 4- to 8-membered heterocycloalkyl, —(CH₂)ᵥ-(optionally substituted 4- to 8-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); each R^{2a} is independently hydrogen, optionally substituted C₁-C₆ alkyl, optionally substituted C₃-C₈ cycloalkyl, or optionally substituted 4- to 8-membered heterocycloalkyl; or two R^{2a} on the same N atom are taken together with the N atom to which they are attached to form a 4- to 8-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); each R^{2a} is independently hydrogen, optionally substituted C₁-C₆ alkyl, optionally substituted C₃-C₁₀ cycloalkyl, or optionally substituted 4- to 10-membered heterocycloalkyl; or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, C₁-C₈ alkyl, C₁-C₈ hydroxyalkyl, optionally substituted C₃-C₈ cycloalkyl, —(CH₂)ᵥ-(optionally substituted C₃-C₈ cycloalkyl), optionally substituted 4- to 8-membered heterocycloalkyl, —(CH₂)ᵥ-(optionally substituted 4- to 8-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); each R^{2a} is independently hydrogen, optionally substituted C₁-C₆ alkyl, optionally substituted C₃-C₈ cycloalkyl, or optionally substituted 4- to 8-membered heterocycloalkyl; or two R^{2a} on the same N atom are taken together with the N atom to which they are attached to form a 4- to 8-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each v is independently 1, 2, or 3. In some embodiments, each v is independently 1 or 2. In some embodiments, each v is 1.

In some embodiments, each R² is independently halogen, —CN, —CH₂CN, —OR^{2a}, —CH₂OR^{2a}, —N(R^{2a})₂, —CH₂N(R^{2a})₂, —C(O)N(R^{2a})₂, —CH₂C(O)N(R^{2a})₂, —N(R^{2a})C(O)NR^{2a}, —CH₂N(R^{2a})C(O)NR^{2a}, —C(O)R^{2b}, —CH₂C(O)R^{2b}, —C(O)OR^{2a}, —CH₂C(O)OR^{2a}, —S(O)₂N(R^{2a})₂, —CH₂S(O)₂N(R^{2a})₂, —S(O)₂R^{2b}, —CH₂S(O)₂R^{2b}, C₁-C₈ alkyl, C₁-C₈ hydroxyalkyl, optionally substituted C₃-C₁₀ cycloalkyl, —CH₂-(optionally substituted C₃-C₁₀ cycloalkyl), optionally substituted 4- to 10-membered heterocycloalkyl, —CH₂-(optionally substituted 4- to 10-membered heterocycloalkyl), optionally substituted phenyl, —CH₂-(optionally substituted phenyl), optionally substituted monocyclic heteroaryl, or —CH₂-(optionally substituted monocyclic heteroaryl); each R^{2a} is independently hydrogen, optionally substituted C₁-C₆ alkyl, optionally substituted C₃-C₁₀ cycloalkyl, or optionally substituted 4- to 10-membered heterocycloalkyl; or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —CH₂CN, —OR^{2a}, —CH₂OR^{2a}, —N(R^{2a})₂, —CH₂N(R^{2a})₂, —C(O)N(R^{2a})₂, —CH₂C(O)N(R^{2a})₂, —N(R^{2a})C(O)NR^{2a}, —CH₂N(R^{2a})C(O)NR^{2a}, —C(O)R^{2b}, —CH₂C(O)R^{2b}, —C(O)OR^{2a}, —CH₂C(O)OR^{2a}, —S(O)₂N(R^{2a})₂, —CH₂S(O)₂N(R^{2a})₂, —S(O)₂R^{2b}—, —CH₂S(O)₂R^{2b}, C₁-C₈ alkyl, C₁-C₈ hydroxyalkyl, optionally substituted C₃-C₈ cycloalkyl, —CH₂-(optionally substituted C₃-C₈ cycloalkyl), optionally substituted 4- to 8-membered heterocycloalkyl, —CH₂-(optionally substituted 4- to 8-membered heterocycloalkyl), optionally substituted phenyl, —CH₂-(optionally substituted phenyl), optionally substituted monocyclic heteroaryl, or —CH₂-(optionally substituted monocyclic heteroaryl); each R^{2a} is independently hydrogen, optionally substituted C₁-C₆ alkyl, optionally substituted C₃-C₈ cycloalkyl, or optionally substituted 4- to 8-membered heterocycloalkyl; or two R^{2a} on the same N atom are taken together with the N atom to which they are attached to form a 4- to 8-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3.

In some embodiments, each R² is independently halogen, —CN, —CH₂CN, —OR^{2a}, —CH₂OR^{2a}, —OCH₂R^{2a}, —N(R^{2a})₂, —CH₂N(R^{2a})₂, —C(O)N(R^{2a})₂, —CH₂C(O)N(R^{2a})₂, —N(R^{2a})C(O)NR^{2a}, —CH₂N(R^{2a})C(O)NR^{2a}, —C(O)R^{2b}, —CH₂C(O)R^{2b}, —C(O)OR^{2a}, —CH₂C(O)OR^{2a}, —S(O)₂N(R^{2a})₂, —CH₂S(O)₂N(R^{2a})₂, —S(O)₂R^{2b}, —CH₂S(O)₂R^{2b}, C₁-C₈ alkyl, C₁-C₈ hydroxyalkyl, optionally substituted C₃-C₈ cycloalkyl, —CH₂-(optionally substituted C₃-C₈ cycloalkyl), optionally substituted 4- to 8-membered heterocycloalkyl, —CH₂-(optionally substituted 4- to 8-membered heterocycloalkyl), optionally substituted phenyl, —CH₂-(optionally substituted phenyl), optionally substituted monocyclic heteroaryl, or —CH₂-(optionally substituted monocyclic heteroaryl); each R^{2a} is independently hydrogen, optionally substituted C₁-C₆ alkyl, optionally substituted C₃-C₈ cycloalkyl, or optionally substituted 4- to 8-membered heterocycloalkyl; or two R^{2a} on the same N atom are taken together with the N atom to which they are attached to form a 4- to 8-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl; and each v is independently 1, 2, or 3. In some embodiments, each R² is independently optionally substituted monocyclic heteroaryl, or —CH₂-(optionally substituted monocyclic heteroaryl). In some embodiments, each R² is independently CH(OH)-pyrazole or pyrole.

In some embodiments, each R² is independently halogen, —CN, —OR^{2a}, —N(R^{2a})₂, —C(O)N(R^{2a})₂, —N(R^{2a})C(O)NR^{2a}, —C(O)R^{2b}, —C(O)OR^{2a}, —S(O)₂N(R^{2a})₂, —S(O)₂R^{2b}, optionally substituted C₁-C₈ alkyl, C₁-C₈ hydroxyalkyl, optionally substituted C₃-C₁₀ cycloalkyl, or optionally substituted 4- to 10-membered heterocycloalkyl; each R^{2a} is independently hydrogen or optionally substituted C₁-C₆ alkyl; and each R^{2b} is independently optionally substituted C₁-C₆ alkyl. In some embodiments, each R² is independently halogen, —CN, —OR^{2a}, —N(R^{2a})₂, —C(O)N(R^{2a})₂, —N(R^{2a})C(O)NR^{2a}, —C(O)R^{2′}, —C(O)OR^{2a}, —S(O)₂N(R^{2a})₂, —S(O)₂R^{2b}, optionally substituted C₁-C₈ alkyl, C₁-C₈ hydroxyalkyl, optionally substituted C₃-C₈ cycloalkyl, or optionally substituted 4- to 8-membered heterocycloalkyl; each R^{2a} is independently hydrogen or optionally substituted C₁-C₆ alkyl; and each R^{2b} is independently optionally substituted C₁-C₆ alkyl. In some embodiments, each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl; each R^{2a} is independently hydrogen or C₁-C₆ alkyl; wherein the alkyl is optionally substituted with 1, 2, or 3 groups independently selected from halogen, —OH, and —CN; or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl; and each R^{2b} is independently C₁-C₆ alkyl. In some embodiments, each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}C₁-C₈ alkyl, or C₁-C₈ hydroxyalkyl; each R^{2a} is independently hydrogen or C₁-C₆ alkyl; and each R^{2b} is independently C₁-C₆ alkyl. In some embodiments, each R² is independently —F, —Cl, —CN, —OR^{2a}, —C(O)N(R^{2a})₂, —S(O)₂R^{2b}, or C₁-C₄ hydroxyalkyl; each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH; or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and each R²¹ is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R² is independently —F, —Cl, —CN, —OR^{2a}, —C(O)N(R^{2a})₂, —S(O)₂R^{2b}, or C₁-C₄ hydroxyalkyl; each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃; and each R²¹ is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, each R² is independently —OR^{2a}, —N(R^{2a})₂, or —C(O)N(R^{2a})₂; and each R^{2a} is independently hydrogen, optionally substituted C₃-C₁₀ cycloalkyl or optionally substituted 4- to 10-membered heterocycloalkyl. In some embodiments, each R² is independently —OR^{2a}, —N(R^{2a})₂, or —C(O)N(R^{2a})₂; and each R^{2a} is independently hydrogen, optionally substituted C₃-C₁₀ cycloalkyl or optionally substituted 4- to 10-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently —OR^{2a}, —N(R^{2a})₂, or —C(O)N(R^{2a})₂; and each R^{2a} is independently hydrogen, optionally substituted C₃-C₈ cycloalkyl or optionally substituted 4- to 8-membered heterocycloalkyl. In some embodiments, each R² is independently —OR^{2a}, —N(R^{2a})₂, or —C(O)N(R^{2a})₂; and each R^{2a} is independently hydrogen, optionally substituted C₃-C₈ cycloalkyl or optionally substituted 4- to 8-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently —N(R^{2a}); and each R^{2a} is independently hydrogen, optionally substituted C₃-C₁₀ cycloalkyl or optionally substituted 4- to 10-membered heterocycloalkyl. In some embodiments, each R² is independently —N(R^{2a})₂; and each R^{2a} is independently hydrogen, optionally substituted C₃-C₁₀ cycloalkyl or optionally substituted 4- to 10-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently —N(R^{2a}); and each R^{2a} is independently hydrogen, optionally substituted C₃-C₈ cycloalkyl or optionally substituted 4 to 8-membered heterocycloalkyl. In some embodiments, each R² is independently —N(R^{2a})₂; and each R^{2a} is independently hydrogen, optionally substituted C₃-C₈ cycloalkyl or optionally substituted 4- to 8-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently

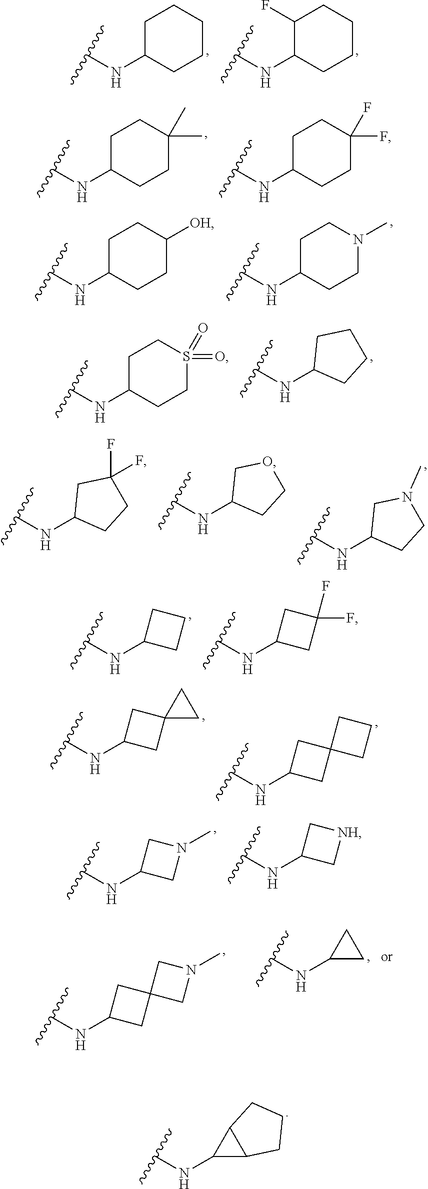

In some embodiments, each R² is independently —OR^{2a} or —O—(CH₂)₁₋₃—R^{2a} and each R^{2a} is independently optionally substituted C₃-C₁₀ cycloalkyl or optionally substituted 4- to 10-membered heterocycloalkyl. In some embodiments, each R² is independently —OR^{2a}; and each R^{2a} is independently optionally substituted C₃-C₁₀ cycloalkyl or optionally substituted 4- to 10-membered heterocycloalkyl. In some embodiments, each R² is independently —OR^{2a}; and each R^{2a} is independently optionally substituted C₃-C₁₀ cycloalkyl or optionally substituted 4- to 10-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently —OR^{2a}; and each R^{2a} is independently optionally substituted C₃-C₈ cycloalkyl or optionally substituted 4- to 8-membered heterocycloalkyl. In some embodiments, each R² is independently —OR^{2a}; and each R^{2a} is independently optionally substituted C₃-C₈ cycloalkyl or optionally substituted 4- to 8-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently

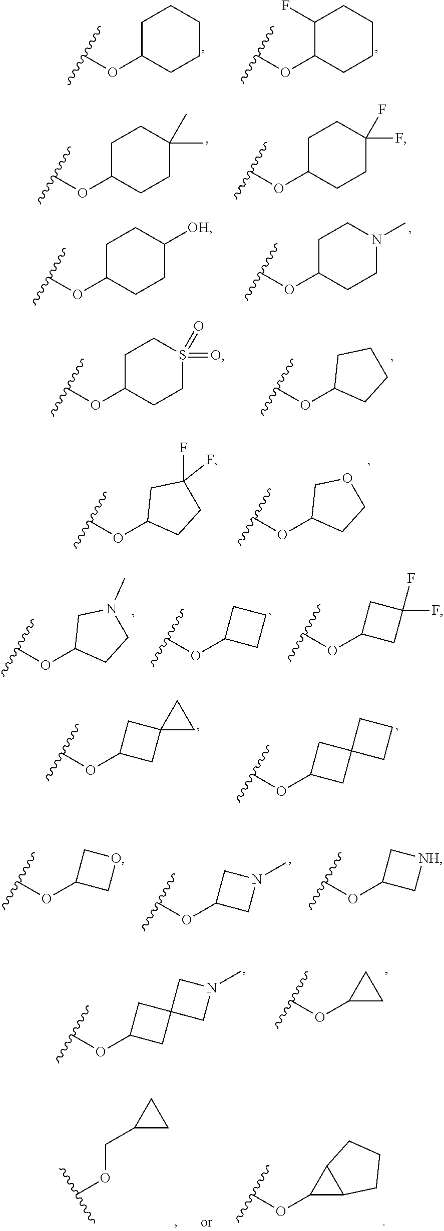

In some embodiments, each R² is independently —OR^{2a}; and each R^{2a} is independently optionally substituted C₃-C₁₀ cycloalkyl or optionally substituted 4- to 10-membered heterocycloalkyl. In some embodiments, each R² is independently —OR^{2a}; and each R^{2a} is independently optionally substituted C₃-C₁₀ cycloalkyl or optionally substituted 4- to 10-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently —OR^{2a}; and each R^{2a} is independently optionally substituted C₃-C₈ cycloalkyl or optionally substituted 4- to 8-membered heterocycloalkyl. In some embodiments, each R² is independently —OR^{2a}; and each R^{2a} is independently optionally substituted C₃-C₈ cycloalkyl or optionally substituted 4- to 8-membered heterocycloalkyl, wherein the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently

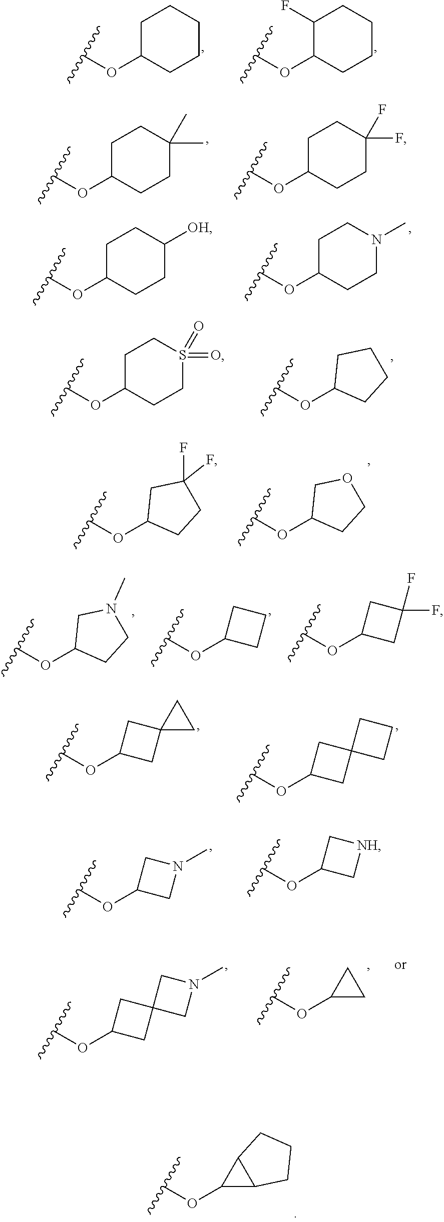

In some embodiments, each R² is independently optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl). In some embodiments, each R² is independently optionally substituted C₃-C₁₀ cycloalkyl, —CH₂-(optionally substituted C₃-C₁₀ cycloalkyl), optionally substituted 4- to 10-membered heterocycloalkyl, —CH₂-(optionally substituted 4- to 10-membered heterocycloalkyl), optionally substituted phenyl, —CH₂-(optionally substituted phenyl), optionally substituted monocyclic heteroaryl, or —CH₂-(optionally substituted monocyclic heteroaryl). In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, —NHC(═O)CH₃, and —P(O)(CH₃)₂.

In some embodiments, each R² is independently optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl). In some embodiments, each R² is independently optionally substituted C₃-C₁₀ cycloalkyl, —CH₂-(optionally substituted C₃-C₁₀ cycloalkyl), optionally substituted 4- to 10-membered heterocycloalkyl, —CH₂-(optionally substituted 4- to 10-membered heterocycloalkyl), optionally substituted phenyl, —CH₂-(optionally substituted phenyl), optionally substituted monocyclic heteroaryl, or —CH₂-(optionally substituted monocyclic heteroaryl). In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently optionally substituted C₃-C₁₀ cycloalkyl, —CH₂-(optionally substituted C₃-C₁₀ cycloalkyl), optionally substituted 4- to 10-membered heterocycloalkyl, or —CH₂-(optionally substituted 4- to 10-membered heterocycloalkyl). In some embodiments, each cycloalkyl is monocyclic or bicyclic cycloalkyl, including fused, bridged, or spirocyclic bicyclic cycloalkyl. In some embodiments, each heterocycloalkyl is monocyclic or bicyclic heterocycloalkyl, including fused, bridged, or spirocyclic bicyclic heterocycloalkyl. In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently optionally substituted C₃-C₈ cycloalkyl, —(CH₂)ᵥ-(optionally substituted C₃-C₈ cycloalkyl), optionally substituted 4- to 8-membered heterocycloalkyl, —(CH₂)ᵥ-(optionally substituted 4- to 8-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl). In some embodiments, each R² is independently optionally substituted C₃-C₈ cycloalkyl, —CH₂-(optionally substituted C₃-C₈ cycloalkyl), optionally substituted 4- to 8-membered heterocycloalkyl, —CH₂-(optionally substituted 4- to 8-membered heterocycloalkyl), optionally substituted phenyl, —CH₂-(optionally substituted phenyl), optionally substituted monocyclic heteroaryl, or —CH₂-(optionally substituted monocyclic heteroaryl). In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, —NHC(═O)CH₃, and —P(O)(CH₃)₂.

In some embodiments, each R² is independently optionally substituted C₃-C₈ cycloalkyl, —(CH₂)ᵥ-(optionally substituted C₃-C₈ cycloalkyl), optionally substituted 4- to 8-membered heterocycloalkyl, —(CH₂)ᵥ-(optionally substituted 4- to 8-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl). In some embodiments, each R² is independently optionally substituted C₃-C₈ cycloalkyl, —CH₂-(optionally substituted C₃-C₈ cycloalkyl), optionally substituted 4- to 8-membered heterocycloalkyl, —CH₂-(optionally substituted 4- to 8-membered heterocycloalkyl), optionally substituted phenyl, —CH₂-(optionally substituted phenyl), optionally substituted monocyclic heteroaryl, or —CH₂-(optionally substituted monocyclic heteroaryl). In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently optionally substituted C₃-C₈ cycloalkyl, —CH₂-(optionally substituted C₃-C₈ cycloalkyl), optionally substituted 4- to 8-membered heterocycloalkyl, or —CH₂-(optionally substituted 4- to 8-membered heterocycloalkyl). In some embodiments, each cycloalkyl is monocyclic or bicyclic cycloalkyl, including fused, bridged, or spirocyclic bicyclic cycloalkyl. In some embodiments, each heterocycloalkyl is monocyclic or bicyclic heterocycloalkyl, including fused, bridged, or spirocyclic bicyclic heterocycloalkyl. In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃.

In some embodiments, each R² is independently optionally substituted C₃-C₁₀ cycloalkyl, optionally substituted 4- to 10-membered heterocycloalkyl, or —CH₂-(optionally substituted 4- to 10-membered heterocycloalkyl). In some embodiments, each R² is independently optionally substituted C₃-C₈ cycloalkyl, optionally substituted 4- to 8-membered heterocycloalkyl, or —CH₂-(optionally substituted 4- to 8-membered heterocycloalkyl). In some embodiments, each cycloalkyl is monocyclic or bicyclic cycloalkyl, including fused, bridged, or spirocyclic bicyclic cycloalkyl. In some embodiments, each heterocycloalkyl is monocyclic or bicyclic heterocycloalkyl, including fused, bridged, or spirocyclic bicyclic heterocycloalkyl. In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently

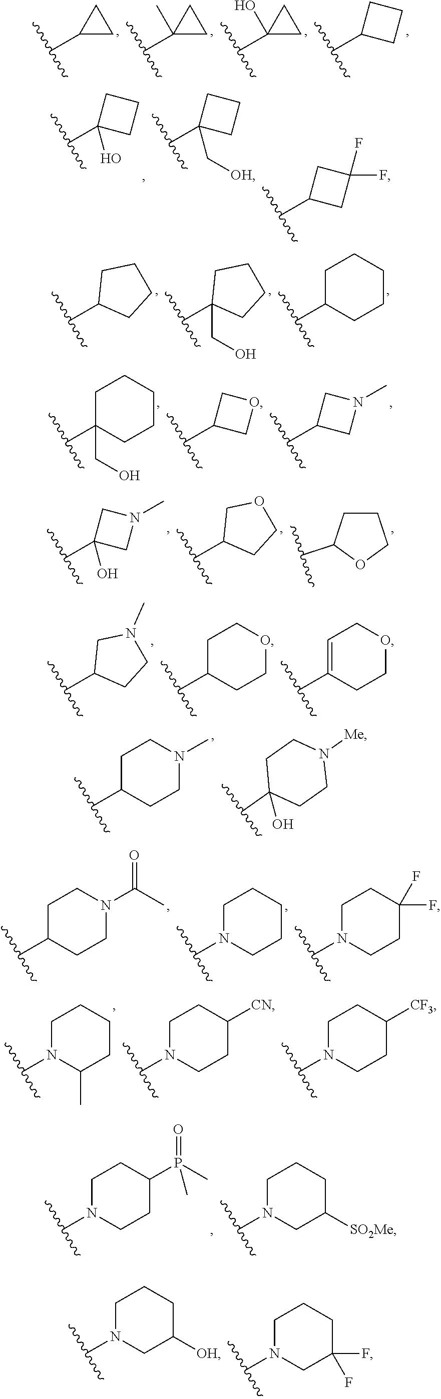

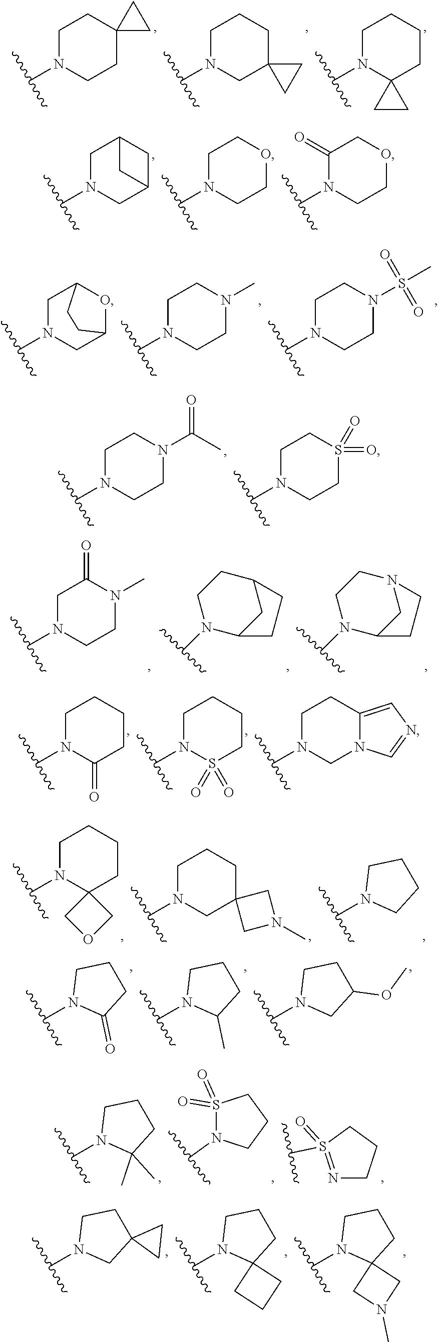

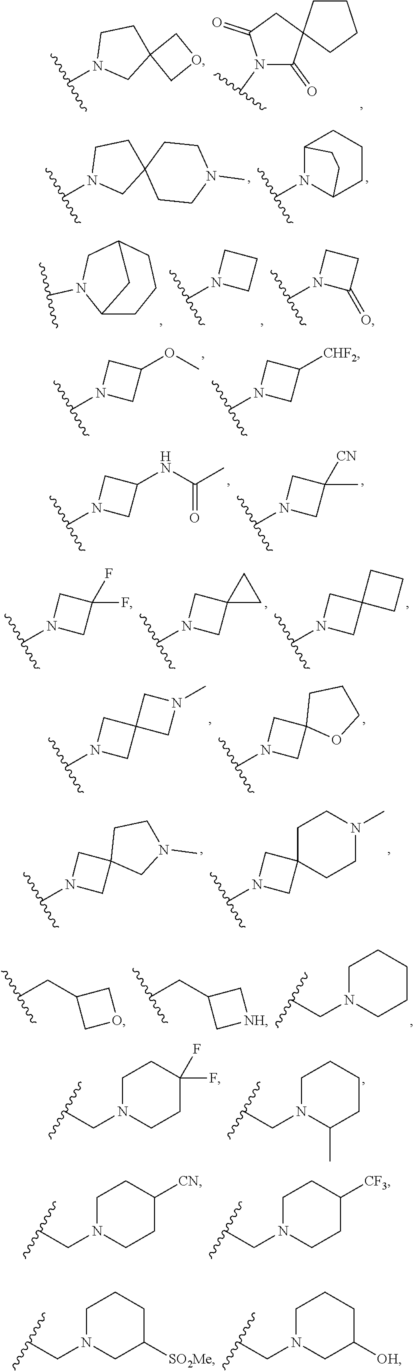

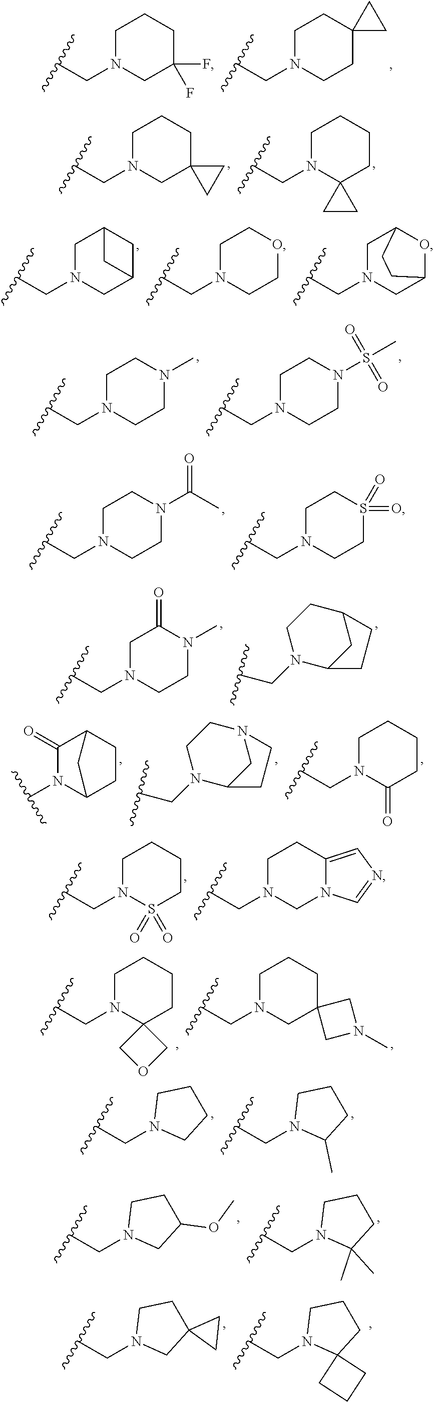

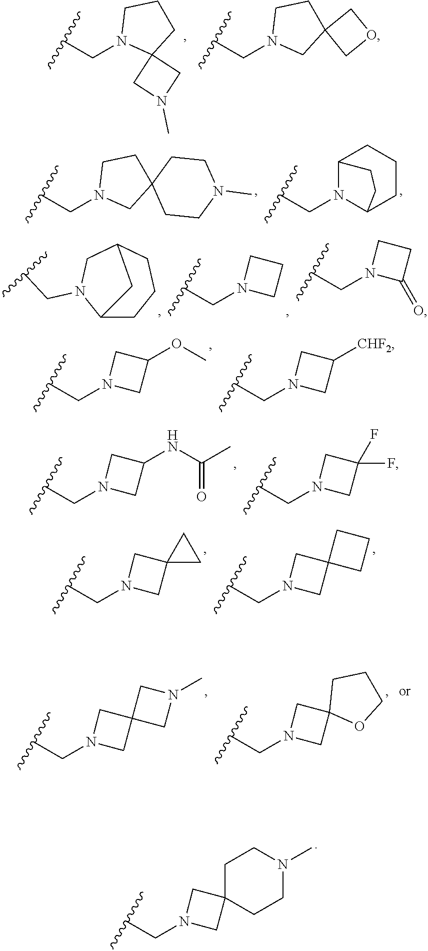

In some embodiments, each R² is independently optionally substituted C₃-C₁₀ cycloalkyl, optionally substituted 4- to 10-membered heterocycloalkyl, or —CH₂-(optionally substituted 4- to 10-membered heterocycloalkyl). In some embodiments, each R² is independently optionally substituted C₃-C₈ cycloalkyl, optionally substituted 4- to 8-membered heterocycloalkyl, or —CH₂-(optionally substituted 4- to 8-membered heterocycloalkyl). In some embodiments, each cycloalkyl is monocyclic or bicyclic cycloalkyl, including fused, bridged, or spirocyclic bicyclic cycloalkyl. In some embodiments, each heterocycloalkyl is monocyclic or bicyclic heterocycloalkyl, including fused, bridged, or spirocyclic bicyclic heterocycloalkyl. In some embodiments, the cycloalkyl or heterocycloalkyl is optionally substituted with 1, 2, or 3 groups independently selected from oxo, —F, —CN, —OH, —OCH₃, —CH₃, —CH₂F, —CHF₂, —CF₃, —SO₂CH₃, —C(═O)CH₃, or —NHC(═O)CH₃. In some embodiments, each R² is independently

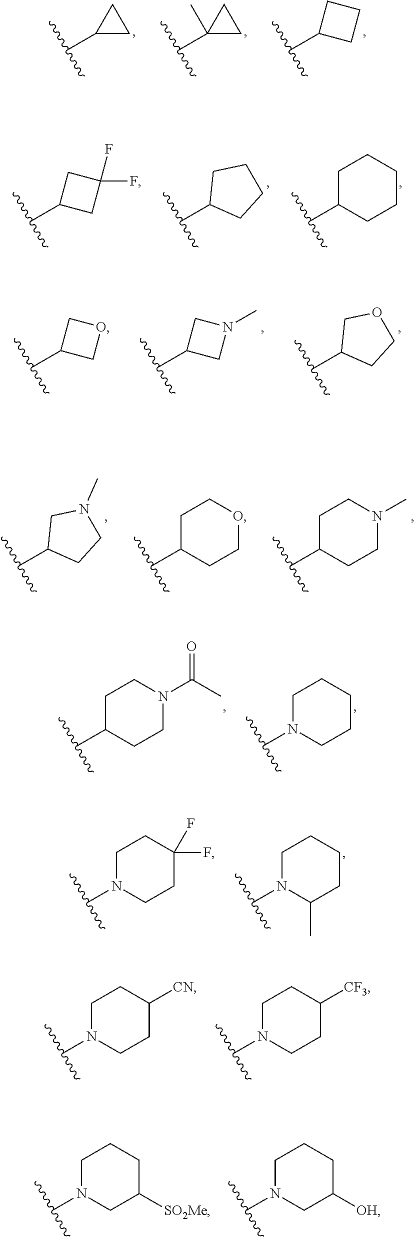

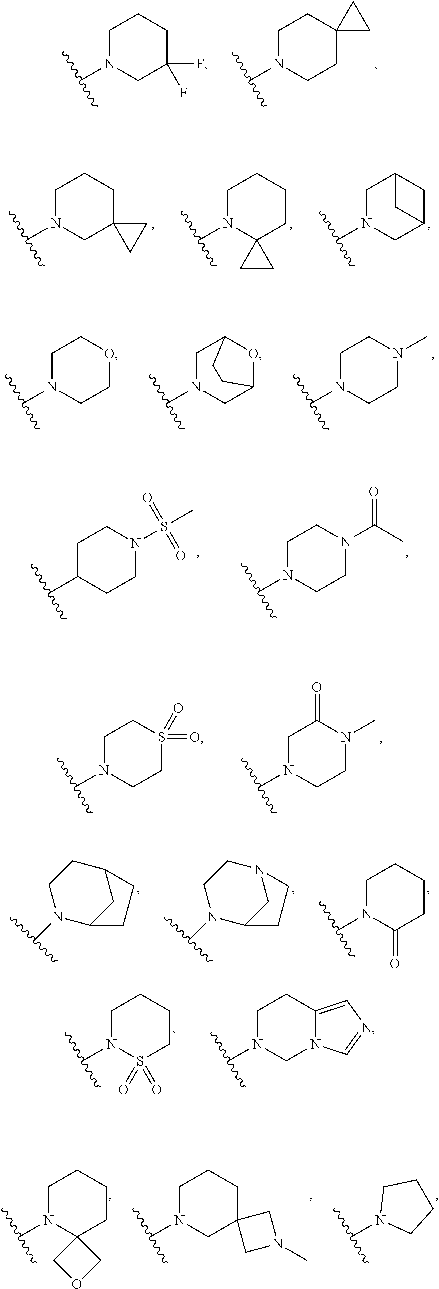

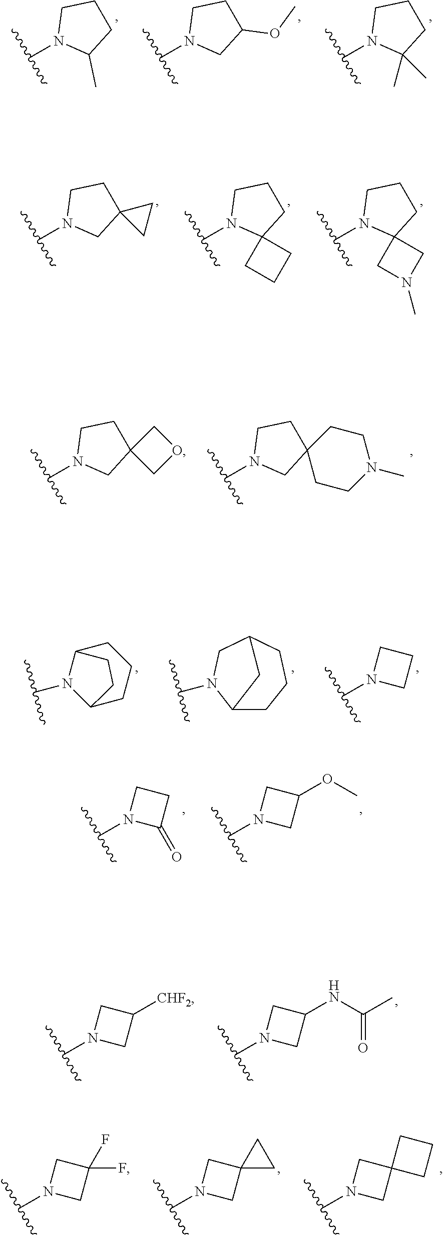

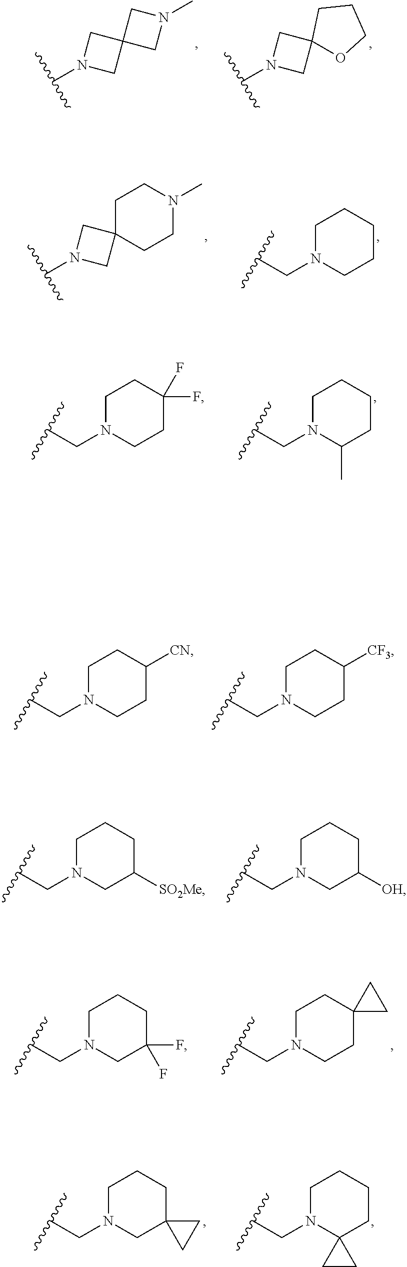

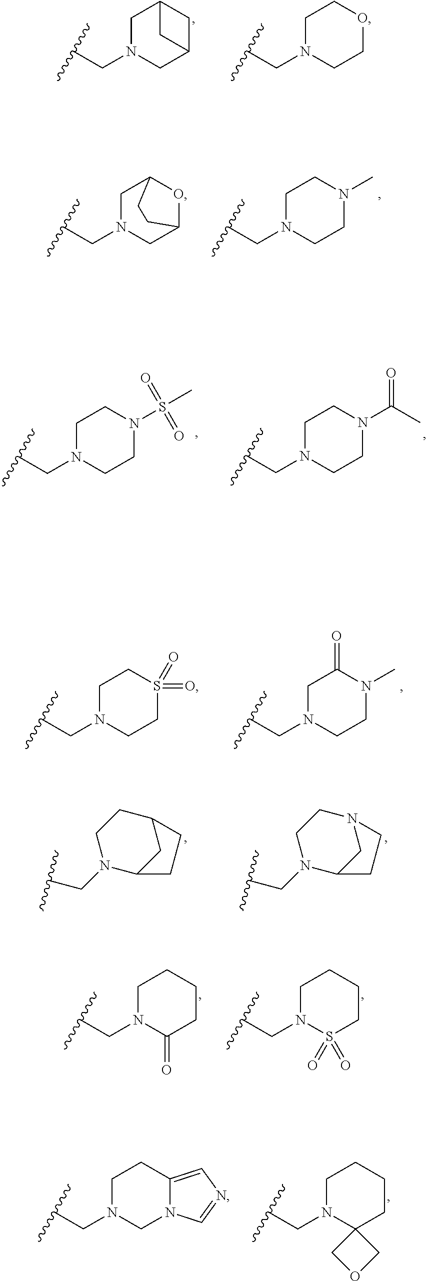

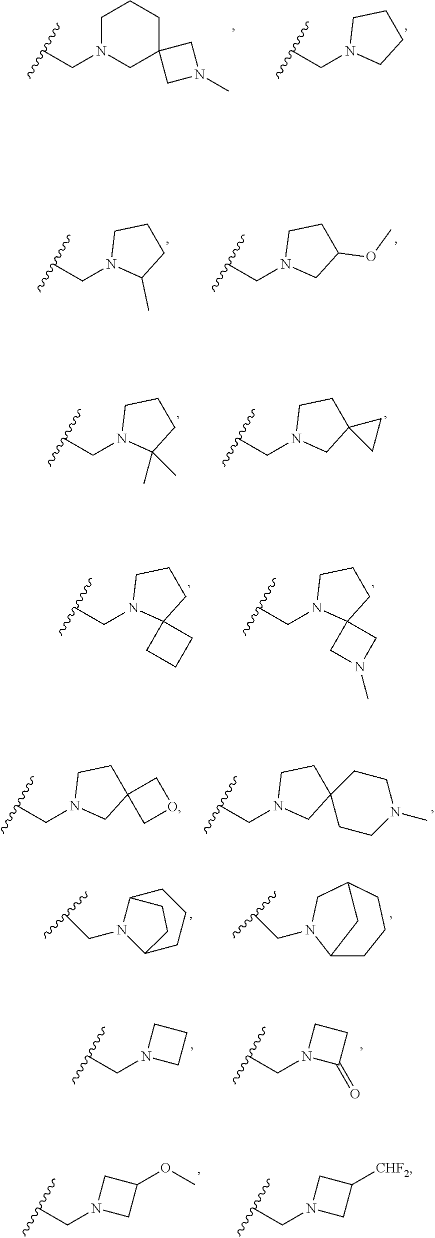

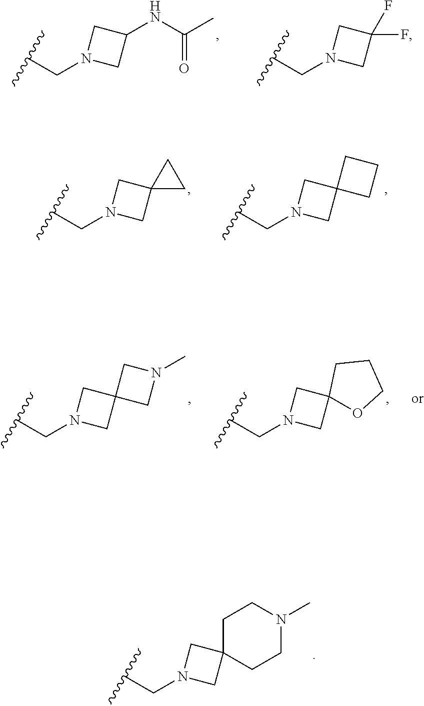

In some embodiments, each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

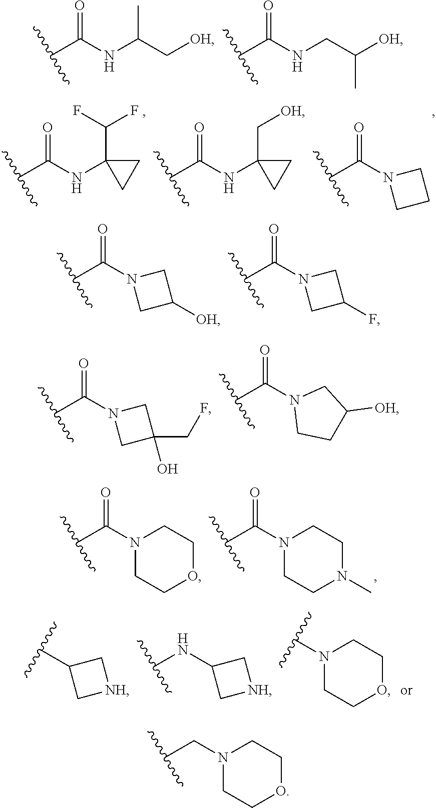

In some embodiments, each R² is independently —Cl, —CN, —OCH₃, —C(O)NH₂, —S(O)₂CH₃, or —CH₂OH.

In some embodiments, each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

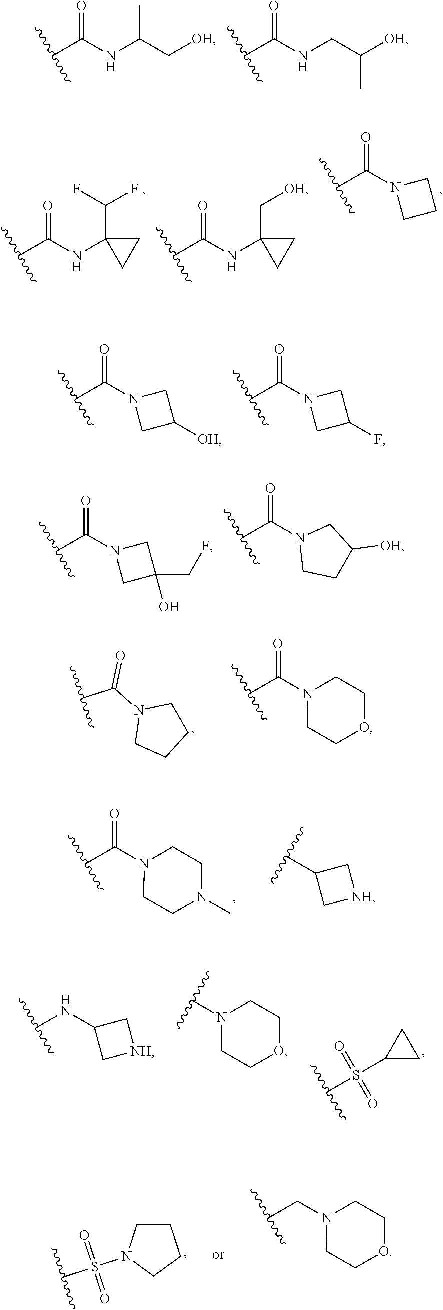

In some embodiments, each R² is independently —Cl, —CN, —OCH₃, —C(O)NH₂, —S(O)₂CH₃, or —CH₂OH.

In some embodiments, m is 0, 1, 2, or 3. In some embodiments, m is 0, 1, or 2. In some embodiments, m is 0. In some embodiments, m is 1. In some embodiments, m is 2. In some embodiments, m is 3.

In some embodiments, when m is 1, v is 1, 2, or 3. In some embodiments, v is 1 or 2. In some embodiments, v is 1. In some embodiments, v is 2. In some embodiments, v is 3.

In some embodiments, each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl. In some embodiments, each R⁴ is independently halogen, —CN, C₁-C₆ alkyl, or C₁-C₆ fluoroalkyl. In some embodiments, each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁴ is independently —CN or —CH₃.

In some embodiments, n is 0, 1, 2, 3, or 4. In some embodiments, n is 0, 1, or 2. In some embodiments, n is 0. In some embodiments, n is 1. In some embodiments, n is 2. In some embodiments, n is 3. In some embodiments, n is 4.

In some embodiments, when n is 2, 3, or 4, two R⁴ on the same carbon atom are taken together with the carbon atom to which they are attached to form a C₃-C₈ cycloalkyl. In some embodiments, two R⁴ on the same carbon atom are taken together with the carbon atom to which they are attached to form a C₃-C₄ cycloalkyl. In some embodiments, two R⁴ on the same carbon atom are taken together with the carbon atom to which they are attached to form a cyclopropyl, cyclobutyl, cyclopentyl, or cyclohexyl. In some embodiments, two R⁴ on the same carbon atom are taken together with the carbon atom to which they are attached to form a cyclopropyl.

In some embodiments, when n is 2, 3, or 4, two R⁴ on different carbon atoms are taken together with the intervening atoms to which they are attached to form a C₁-C₃ alkylene bridge. In some embodiments, two R⁴ on different carbon atoms are taken together with the intervening atoms to which they are attached to form a C₁ alkylene bridge. In some embodiments, two R⁴ on different carbon atoms are taken together with the intervening atoms to which they are attached to form a C₂ alkylene bridge. In some embodiments, two R⁴ on different carbon atoms are taken together with the intervening atoms to which they are attached to form a C₃ alkylene bridge.

In some embodiments, R³ is —N(R^{3a})₂, —NR^{3a}(SO₂R^{3b}), —SO₂NR^{3a}R^{3b}, —SO(═NH)NR^{3a}R^{3b}, —NR^{3a}SO₂N(R^{3a})₂, —SO₂R^{3b}, —NR^{3a}C(O)—C(O)R^{3b}, —NR^{3a}C(O)OR^{3b}, or —NR^{3b}C(O)NR^{3a}R^{3a}; each R^{3a} is independently hydrogen, optionally substituted C₁-C₆ alkyl, or C₁-C₆ hydroxyalkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl.

In some embodiments, R³ is —N(R^{3a})₂, —NR^{3a}(SO₂R^{3b}), —SO₂NR^{3a}R^{3b}, —NR^{3a}SO(═NH)R^{3b}, —NR^{3a}SO₂N(R^{3a})₂, —SO₂R^{3b}, or —NR^{3a}C(O)—C(O)R^{3b}, —NR^{3a}C(O)OR^{3b}, or —NR^{3b}C(O)NR^{3a}R^{3a}; each R^{3a} is independently hydrogen, C₁-C₆ alkyl, or C₁-C₆ hydroxyalkyl; R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl.

In some embodiments, R³ is —NR^{3a}(SO₂R^{3b}), —SO₂NR^{3a}R^{3b}, —SO(═NH)NR^{3a}R^{3b}, —NR^{3a}C(O)OR^{3b}, or —NR^{3b}C(O)NR^{3a}R^{3a}; R^{3a} is hydrogen, C₁-C₆ alkyl, or hydroxyalkyl; and R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl; or R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}) or —SO₂NR^{3a}R^{3b}; R^{3a} is hydrogen, C₁-C₆ alkyl, or C₁-C₃ hydroxyalkyl; and R^{3b} is optionally substituted C₁-C₆ alkyl, C₁-C₆ hydroxyalkyl, optionally substituted C₂-C₆ alkynyl, optionally substituted C₃-C₆ cycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl).

In some embodiments, R^{3a} is hydrogen, C₁-C₃ alkyl, or C₁-C₃ hydroxyalkyl; and R^{3b} is optionally substituted C₁-C₃ alkyl, C₁-C₃ hydroxyalkyl, C₃-C₆ cycloalkyl which is substituted by a C₁-C₃ alkyl or C₁-C₃ hydroxyalkyl, or (C₃-C₁₀ cycloalkyl)-(C₁-C₆ alkyl) which is optionally substituted by C₁-C₃ alkyl or C₁-C₃ hydroxyalkyl.

In some embodiments, R^{3a} is hydrogen, —CH₃, —CH₂CH₃, or —CH₂CH₂OH; and R^{3b} is —CH₃, —CHF₂, —CH₂CH₃, —CH₂CN, —CH₂CH₂CH₃, —CH(CH₃)₂, —CH₂OH, —CH₂CH₂OH, —CH₂CH₂OCH₃, —CH₂CH₂CH₂OH, —CH(CH₃)CH₂OH, —CH₂CH₂C(CH₃)₂OH, —CH₂C≡CCH₃, —CH₂CH₂C≡CH, —CH₂CH₂CH₂CH₂C≡CH,

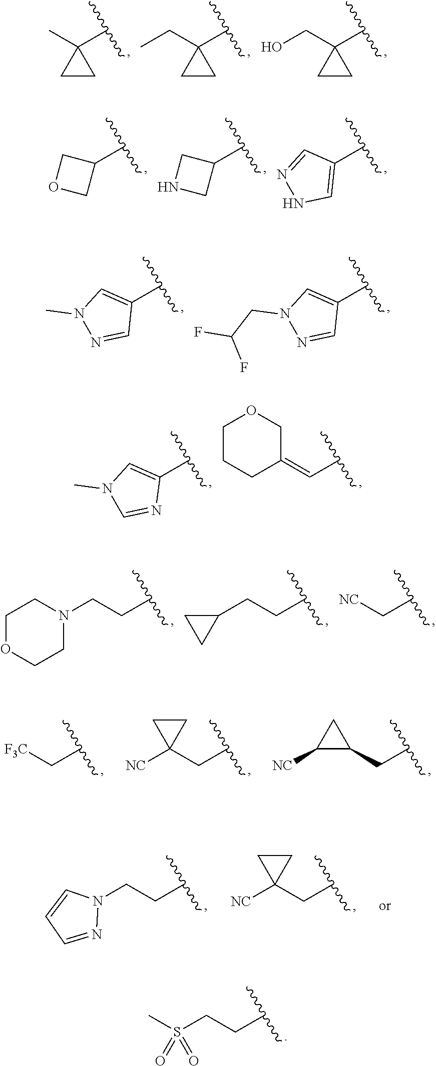

In some embodiments, R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}); R^{3a} and R^{3b} are taken together with the intervening atoms to which they are attached to form an optionally substituted 5- to 6-membered heterocycloalkyl.

In some embodiments, R³ is —NR^{3a}C(O)OR^{3b}, or —NR^{3b}C(O)NR^{3a}R^{3a}; R^{3a} and R^{3b} are taken together with the intervening atoms to which they are attached to form an optionally substituted 5- to 6-membered heterocycloalkyl.

In some embodiments, R^{3a} and R^{3b} are taken together with the intervening atoms to which they are attached to form

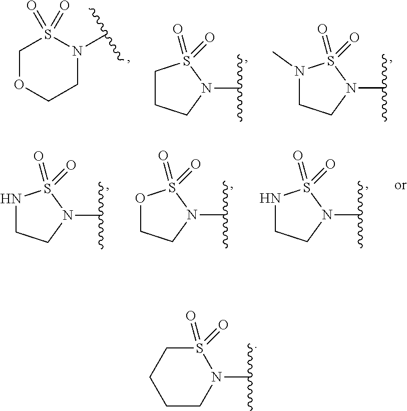

In some embodiments, R³ is —N(R^{3a})₂; and each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, R³ is —SO₂R^{3b}; and R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), optionally substituted monocyclic 5-membered heteroaryl, or optionally substituted (heteroaryl)-(C₁-C₆ alkyl).

In some embodiments, R^{3b} is C₁-C₃ alkyl, C₁-C₃ hydroxyalkyl, C₂-C₆ alkynyl, or C₃-C₆ cycloalkyl which is substituted by a —CN, C₁-C₃ alkyl or C₁-C₃ hydroxyalkyl.

In some embodiments, R^{3b} is —CH₃, —CH₂CH₃, —CH₂CH₂CH₃, —CH(CH₃)₂, —CH₂OH, —CH₂CN, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂, —CH₂C≡CCH₃,

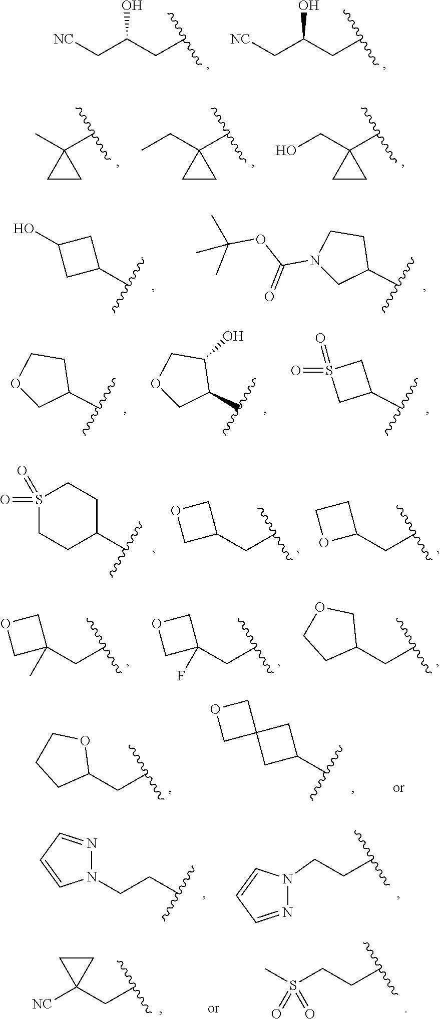

In some embodiments, R³ is —NR^{3a}C(O)—C(O)R^{3b}; R^{3a} is hydrogen or C₁-C₆ alkyl; and R^{3b} is C₁-C₆ alkyl, C₁-C₆ hydroxyalkyl, or optionally substituted C₃-C₆ cycloalkyl.

In some embodiments, R^{3a} is hydrogen, —CH₃, —CH₂CH₃, —CH₂CH₂CH₃, or —CH(CH₃)₂; and R^{3b} is —CH₃, —CH₂CH₃, —CH₂CH₂CH₃, —CH(CH₃)₂, —CH₂OH, —CH₂CH₂OH, —CH₂CH₂CH₂OH,

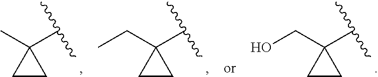

In some embodiments, R³ is —NH₂, —NHSO₂CH₃, —NHSO₂CHF₂, —NHSO₂CH₂CH₃, —N(CH₃)SO₂CH₃, —N(CH₃)SO₂CH₂CH₃, —NHSO₂CH₂CH₂OH, —NHSO₂CH₂CH₂OCH₃, —N(CH₃)SO₂CH₂CH₂OH, —NHSO₂CH(CH₃)CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂CH₂CH₂OCH₃, —SO₂CH₂CH₂CH₂OH, —SO₂CH₂C(CH₃)₂OH, —SO₂CH₂ CH₂C(CH₃)₂OH, —SO₂CH₂CH₂C(CH₃)₂OH, —SO₂CH(CH₃)CH₂OH, —SO₂CH₂CH₂SO₂CH₃, —SO₂CH₂CH₂OCH₂CN, —SO₂CH₂CH(CH₃)OH, —SO₂CH₂CN, —SO₂CH₂CH₂CN, —SO₂CH(CH₃)CN, —SO₂CH₂CH₂C(═CH₂)CN, —SO₂CH₂C(CH₃)(OH)CH₂OH, —SO₂CH₂C(═O)NH₂, —SO₂NHCH₃, —SO₂NHCH₂CH₃, —SO₂NHCH₂CH₂OH, —SO₂CH₂C≡CCH₃,

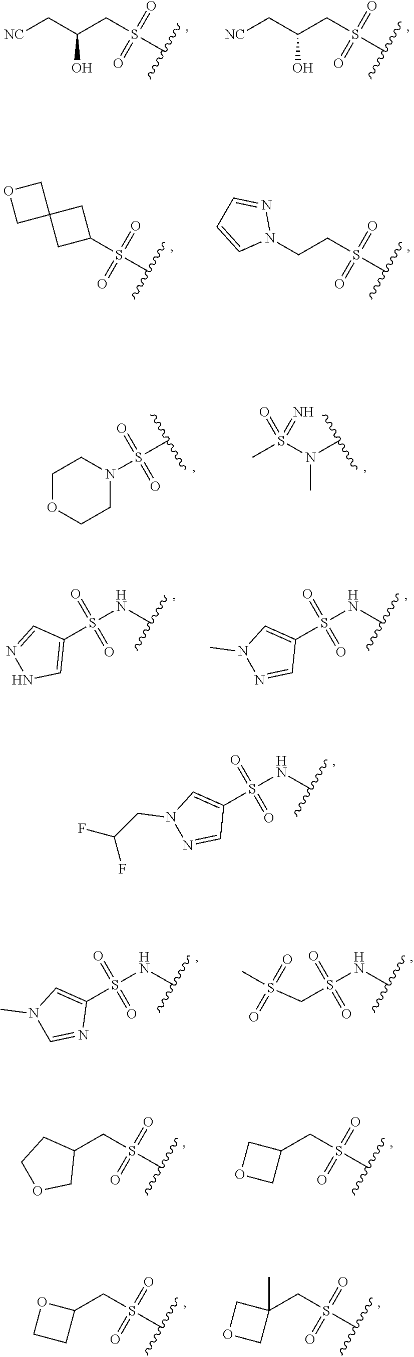

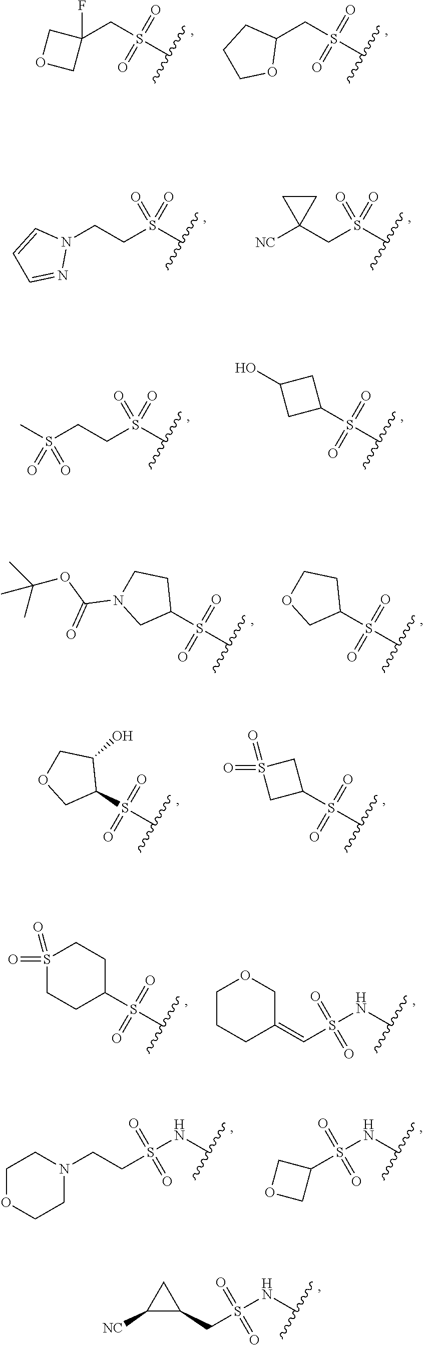

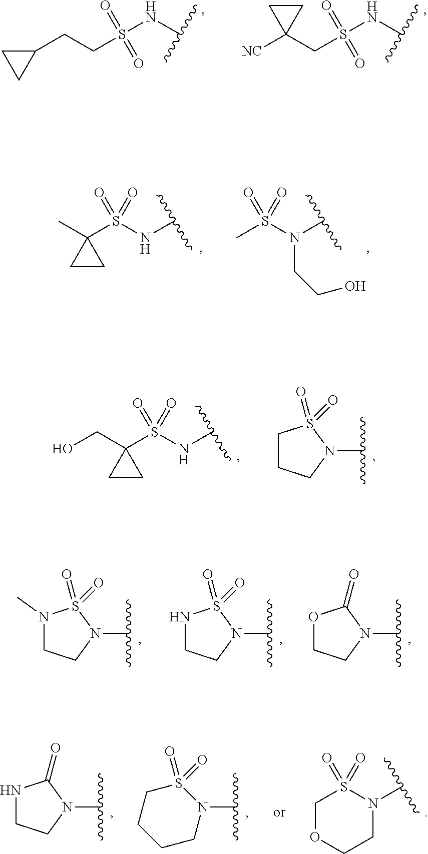

In some embodiments, R³ is —N(R^{3a})₂, —NR^{3a}(SO₂R^{3b}), —SO₂NR^{3a}R^{3b}, —NR^{3a}SO₂N(R^{3a})₂, or —SO₂R^{3b}; each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl.

In some embodiments, R³ is —N(R^{3a})₂, —SO₂NR^{3a}R^{3b}, —NR^{3a}(SO₂R^{3b}), —NR^{3a}SO₂N(R^{3a})₂, —SO₂R^{3b}, or —NR^{3a}C(O)—C(O)R^{3b}; each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl. In some embodiments, R³ is —N(R^{3a})₂, —SO₂NR^{3a}R^{3b}, NR^{3a}(SO₂R^{3b}), —NR^{3a}SO₂N(R^{3a})₂, —SO₂R^{3b}, or —NR^{3a}C(O)—C(O)R^{3b}; each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl. In some embodiments, R³ is —N(R^{3a})₂, —SO₂NR^{3a}R^{3b}, —NR^{3a}(SO₂R^{3b}), —NR^{3a}SO₂N(R^{3a})₂, —SO₂R^{3b}, or —NR^{3a}C(O)—C(O)R^{3b}; each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl. In some embodiments, R³ is —N(R^{3a})₂, —NR^{3a}(SO₂R^{3b}), —SO₂NR^{3a}R^{3b}, —SO₂R^{3b}, or —NR^{3a}C(O)—C(O)R^{3b}; each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, or optionally substituted C₃-C₆ cycloalkyl; or R^{3a} and R^{3b} are taken together with the intervening atoms to which they are attached to form an optionally substituted 4- to 8-membered heterocycloalkyl. In some embodiments, R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b}; each R^{3a} is independently hydrogen or C₁-C₆ alkyl; R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl. In some embodiments, R³ is —N(R^{3a})₂, —NR^{3a}(SO₂R^{3b})—SO₂NR^{3a}R^{3b}, —NR^{3a}SO₂N(R^{3a})₂, —SO₂R^{3b}, or —NR^{3a}C(O)—C(O)R^{3b}; each R^{3a} is independently hydrogen or C₁-C₆ alkyl; R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl; or R^{3a} and R^{3b} are taken together with the intervening atoms to which they are attached to form an optionally substituted 4- to 8-membered heterocycloalkyl. In some embodiments, R³ is —N(R^{3a})₂, —NR^{3a}(SO₂R^{3b})—SO₂NR^{3a}R^{3b}, —SO₂R^{3b}, or —NR^{3a}C(O)—C(O)R^{3b}; each R^{3a} is independently hydrogen or C₁-C₆ alkyl; R^{3b} is C₁-C₆ alkyl, C₁-C₆ hydroxyalkyl, or optionally substituted C₃-C₆ cycloalkyl; or R^{3a} and R^{3b} are taken together with the intervening atoms to which they are attached to form an optionally substituted 4- to 8-membered heterocycloalkyl.

In some embodiments, R³ is —N(R^{3a})₂. In some embodiments, R³ is —NR^{3a}(SO₂R^{3b}). In some embodiments, R³ is —SO₂NR^{3a}R^{3b}. In some embodiments, R³ is —NR^{3a}SO₂N(R^{3a})₂. In some embodiments, R³ is —SO₂R^{3b}. In some embodiments, R³ is —NR^{3a}C(O)—C(O)R^{3b}.

In some embodiments, each R^{3a} is independently hydrogen or C₁-C₆ alkyl. In some embodiments, each R^{3a} is independently hydrogen or C₁-C₃ alkyl. In some embodiments, each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{3a} is independently hydrogen, —CH₃, —CH₂CH₃, —CH₂CH₂CH₃, or —CH(CH₃)₂. In some embodiments, each R^{3a} is independently hydrogen, —CH₃, or —CH₂CH₃. In some embodiments, each R^{3a} is hydrogen or —CH₃. In some embodiments, each R^{3a} is hydrogen.

In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl. In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl. In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl. In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl. In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic 5-membered heteroaryl. In some embodiments, R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂,

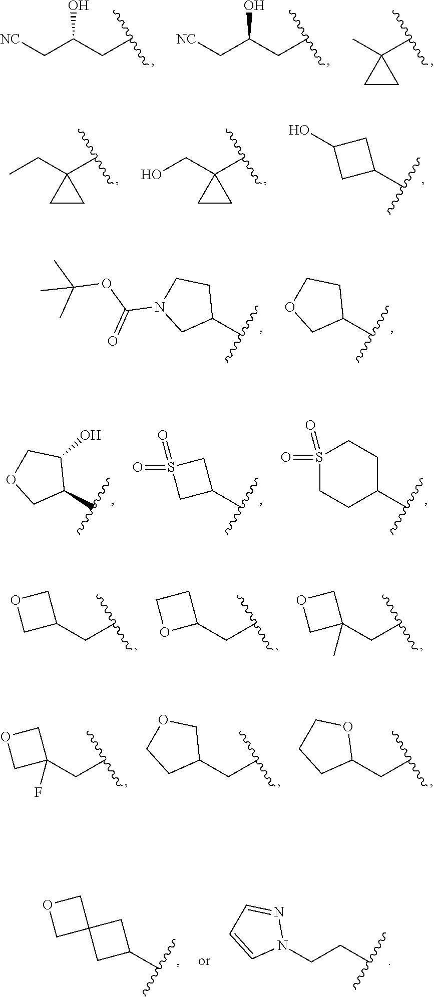

In some embodiments, R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH,

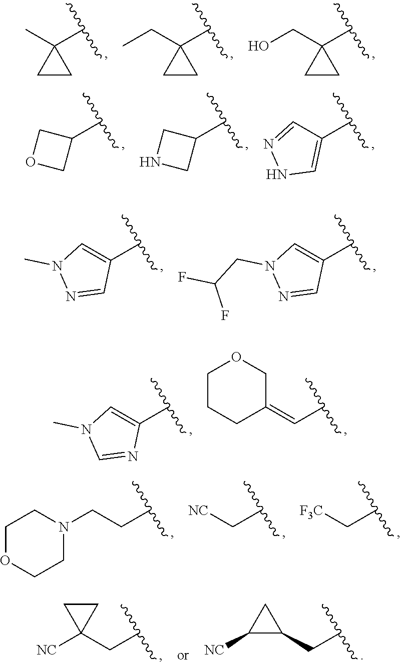

In some embodiments, R^{3b} is C₁-C₆ alkyl, C₁-C₆ hydroxyalkyl, or optionally substituted C₃-C₆ cycloalkyl. In some embodiments, R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl. In some embodiments, R^{3b} is —CH₃, —CH₂CH₃, —CH₂CH₂CH₃, —CH(CH₃)₂, —CH₂OH, —CH₂CH₂OH, —CH₂CH₂CH₂OH,

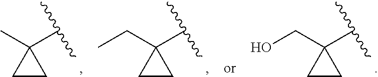

In some embodiments, R³ is —NR^{3a}(SO₂R^{3b}) or —SO₂NR^{3a}R^{3b}. In some such embodiments, R^{3a} is hydrogen or C₁-C₆ alkyl; and R^{3b} is C₁-C₆ alkyl, C₁-C₆ hydroxyalkyl, or optionally substituted C₃-C₆ cycloalkyl. In other such embodiments, R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}). In some such embodiments, R^{3a} is hydrogen or C₁-C₆ alkyl; and R^{3b} is C₁-C₆ alkyl, C₁-C₆ hydroxyalkyl, or optionally substituted C₃-C₆ cycloalkyl. In other such embodiments, R^{3a} and R^{3b} are taken together with the intervening atoms to which they are attached to form an optionally substituted 5- to 6-membered heterocycloalkyl.

In some embodiments, R³ is —SO₂NR^{3a}R^{3b}. In some such embodiments, R^{3a} is hydrogen or C₁-C₆ alkyl; and R^{3b} is C₁-C₆ alkyl, C₁-C₆ hydroxyalkyl, or optionally substituted C₃-C₆ cycloalkyl. In other such embodiments, R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}) or —SO₂NR^{3a}R^{3b}; R^{3a} is hydrogen or C₁-C₆ alkyl; and R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl; or R^{3a} and R^{3b} are taken together with the intervening atoms to which they are attached to form an optionally substituted 5- to 6-membered heterocycloalkyl. In some embodiments, R³ is —NR^{3a}(SO₂R^{3b}) or —SO₂NR^{3a}R^{3b}; R^{3a} is hydrogen or C₁-C₆ alkyl; and R^{3b} is C₁-C₆ alkyl, C₁-C₆ hydroxyalkyl, or optionally substituted C₃-C₆ cycloalkyl; or R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}); R^{3a} is hydrogen or C₁-C₆ alkyl; and R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl; or R^{3a} and R^{3b} are taken together with the intervening atoms to which they are attached to form an optionally substituted 5- to 6-membered heterocycloalkyl. In some embodiments, R³ is —NR^{3a}(SO₂R³); R^{3a} is hydrogen or C₁-C₆ alkyl; and R^{3b} is C₁-C₆ alkyl, C₁-C₆ hydroxyalkyl, or optionally substituted C₃-C₆ cycloalkyl; or R^{3a} and R^{3b} are taken together with the intervening atoms to which they are attached to form an optionally substituted 5- to 6-membered heterocycloalkyl.

In some embodiments, R³ is —SO₂NR^{3a}R^{3b}; R^{3a} is hydrogen or C₁-C₆ alkyl; and R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl; or R^{3a} and R^{3b} are taken together with the intervening atoms to which they are attached to form an optionally substituted 5- to 6-membered heterocycloalkyl. In some embodiments, R³ is —SO₂NR^{3a}R^{3b}; R^{3a} is hydrogen or C₁-C₆ alkyl; and R^{3b} is C₁-C₆ alkyl, C₁-C₆ hydroxyalkyl, or optionally substituted C₃-C₆ cycloalkyl; or R^{3a} and R^{3b} are taken together with the intervening atoms to which they are attached to form an optionally substituted 5- to 6-membered heterocycloalkyl.

In some embodiments, R³ is —NR^{3a}(SO₂R^{3b}) or —SO₂NR^{3a}R^{3b}; R^{3a} is hydrogen or C₁-C₆ alkyl; and R^{3b} is C₁-C₆ alkyl, C₁-C₆ hydroxyalkyl, or optionally substituted C₃-C₆ cycloalkyl. In some embodiments, R³ is —NR^{3a}(SO₂R^{3b}); R^{3a} is hydrogen or C₁-C₆ alkyl; and R^{3b} is C₁-C₆ alkyl, C₁-C₆ hydroxyalkyl, or optionally substituted C₃-C₆ cycloalkyl. In some embodiments, R³ is —SO₂NR^{3a}R^{3b}; R^{3a} is hydrogen or C₁-C₆ alkyl; and R^{3b} is C₁-C₆ alkyl, C₁-C₆ hydroxyalkyl, or optionally substituted C₃-C₆ cycloalkyl. In some embodiments, R^{3a} is hydrogen or C₁-C₃ alkyl; and R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.

In some embodiments, R^{3a} is hydrogen, —CH₃, or —CH₂CH₃; and R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH, —CH₂CH₂CH₂CH₂C≡CH,

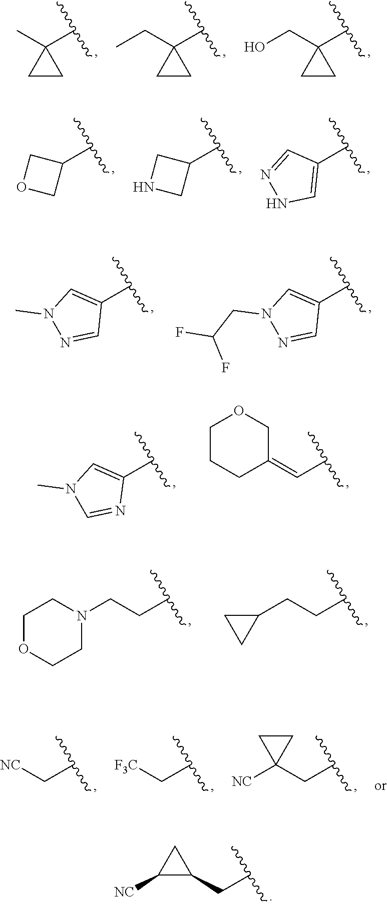

In some embodiments, R^{3a} is hydrogen, —CH₃, or —CH₂CH₃; and R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH,

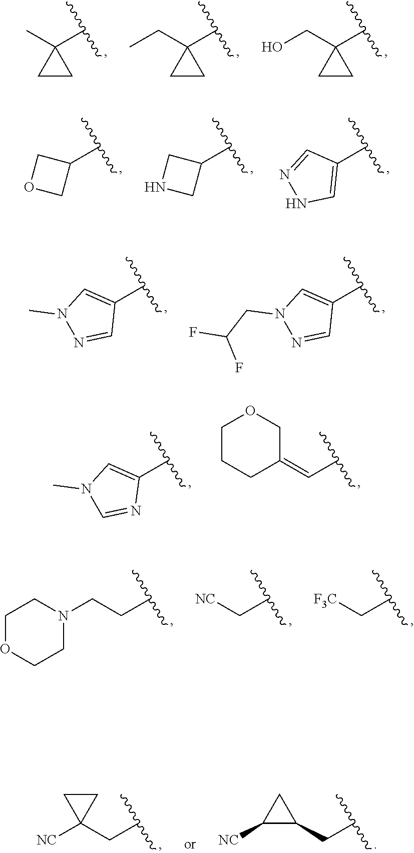

In some embodiments, R^{3a} is hydrogen, —CH₃, or —CH₂CH₃; and R^{3b} is —CH₃, —CH₂CH₃, —CH₂CH₂CH₃, —CH(CH₃)₂, —CH₂OH, —CH₂CH₂OH, —CH₂CH₂CH₂OH,

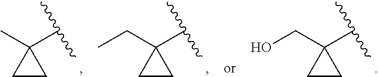

In some embodiments, R³ is —NR^{3a}(SO₂R^{3b}) or —SO₂NR^{3a}R^{3b}; and R^{3a} and R^{3b} are taken together with the intervening atoms to which they are attached to form an optionally substituted 5- to 6-membered heterocycloalkyl. In some embodiments, R³ is —NR^{3a}(SO₂R^{3b}); and R^{3a} and R^{3b} are taken together with the intervening atoms to which they are attached to form an optionally substituted 5- to 6-membered heterocycloalkyl. In some embodiments, R³ is —SO₂NR^{3a}R^{3b}; and R^{3a} and R^{3b} are taken together with the intervening atoms to which they are attached to form an optionally substituted 5- to 6-membered heterocycloalkyl. In some embodiments, R^{3a} and R^{3b} are taken together with the intervening atoms to which they are attached to form

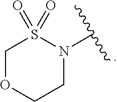 [SMILES: *N1CCOCS1(=O)=O]

In some embodiments, R^{3a} and R^{3b} are taken together with the intervening atoms to which they are attached to form

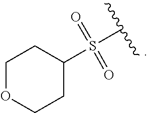 [SMILES: *S(=O)(=O)C1CCOCC1]

In some embodiments, R³ is —N(R^{3a})₂. In some such embodiments, each R^{3a} is independently hydrogen or C₁-C₆ alkyl. In some embodiments, each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{3a} is hydrogen or —CH₃. In some embodiments, each R^{3a} is hydrogen.

In some embodiments, R³ is —SO₂R^{3b}. In some embodiments, R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic 5-membered heteroaryl. In some embodiments, R^{3b} is C₁-C₆ alkyl, C₁-C₆ hydroxyalkyl, or optionally substituted C₃-C₆ cycloalkyl. In some embodiments, R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl. In some embodiments, R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂,

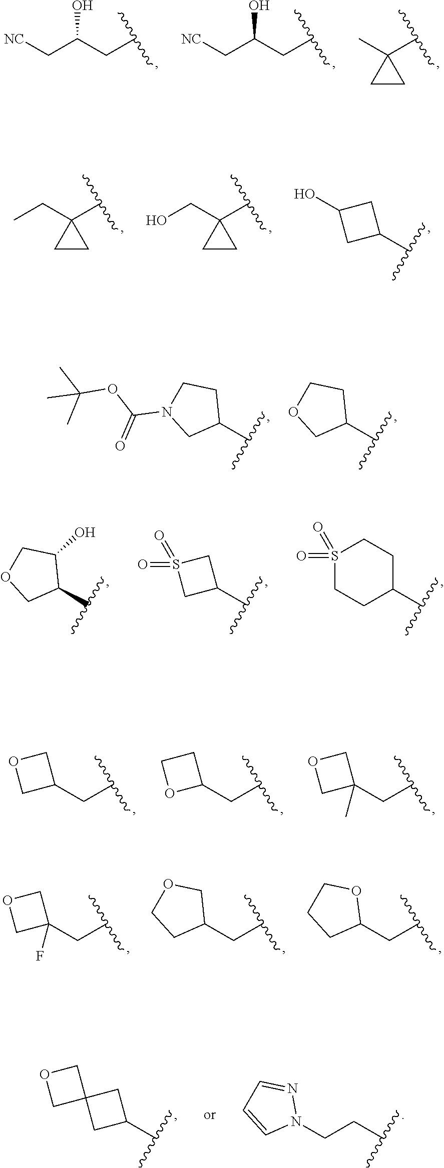

In some embodiments, R^{3b} is —CH₃, —CH₂CH₃, —CH₂CH₂CH₃, —CH(CH₃)₂, —CH₂OH, —CH₂CH₂OH, —CH₂CH₂CH₂OH,

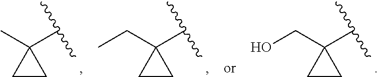

In some embodiments, R³ is —NR^{3a}C(O)—C(O)R^{3b}. In some embodiments, R^{3a} is hydrogen or C₁-C₆ alkyl; and R^{3b} is C₁-C₆ alkyl, C₁-C₆ hydroxyalkyl, or optionally substituted C₃-C₆ cycloalkyl. In some embodiments, R^{3a} is hydrogen, —CH₃, —CH₂CH₃, —CH₂CH₂CH₃, or —CH(CH₃)₂; and R^{3b} is —CH₃, —CH₂CH₃, —CH₂CH₂CH₃, —CH(CH₃)₂, —CH₂OH, —CH₂CH₂OH, —CH₂CH₂CH₂OH,

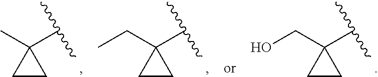

In some embodiments, R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂CH₂CH₂OCH₃, —SO₂CH₂CH₂CH₂OH, —SO₂CH₂C(CH₃)₂OH, —SO₂CH₂CH₂C(CH₃)₂OH, —SO₂CH(CH₃)CH₂OH, —SO₂CH₂CH₂SO₂CH₃, —SO₂CH₂CH₂OCH₂CN, —SO₂CH₂CH(CH₃)OH, —SO₂CH₂CH₂CN, —SO₂CH(CH₃)CN, —SO₂CH₂CH₂C(═CH₂)CN, —SO₂CH₂C(CH₃)(OH)CH₂OH, —SO₂CH₂C(═O)NH₂, —SO₂NHCH₂CH₂OH,

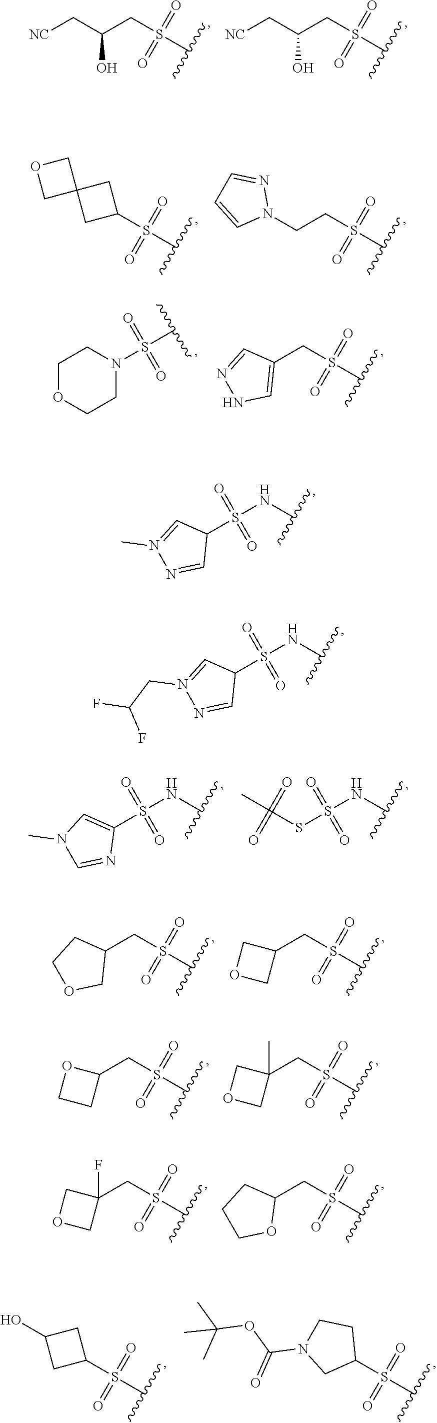

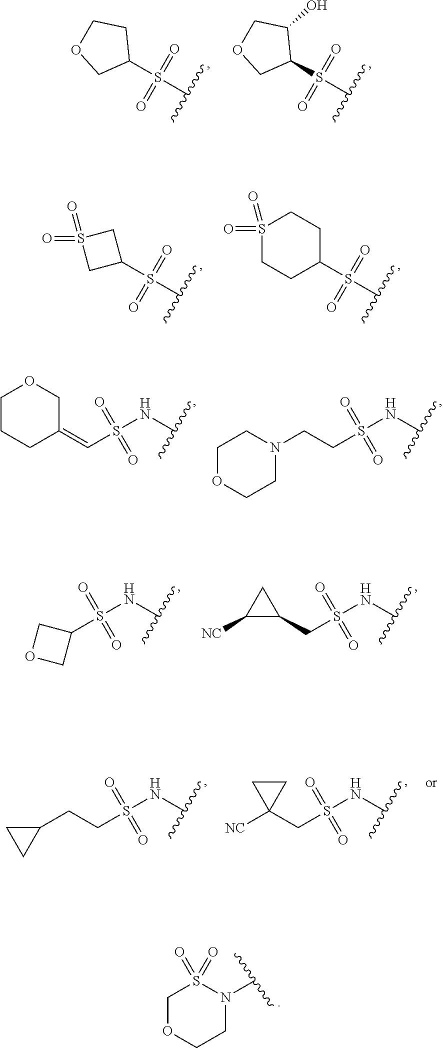

In some embodiments, R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂NHCH₂CH₂OH,

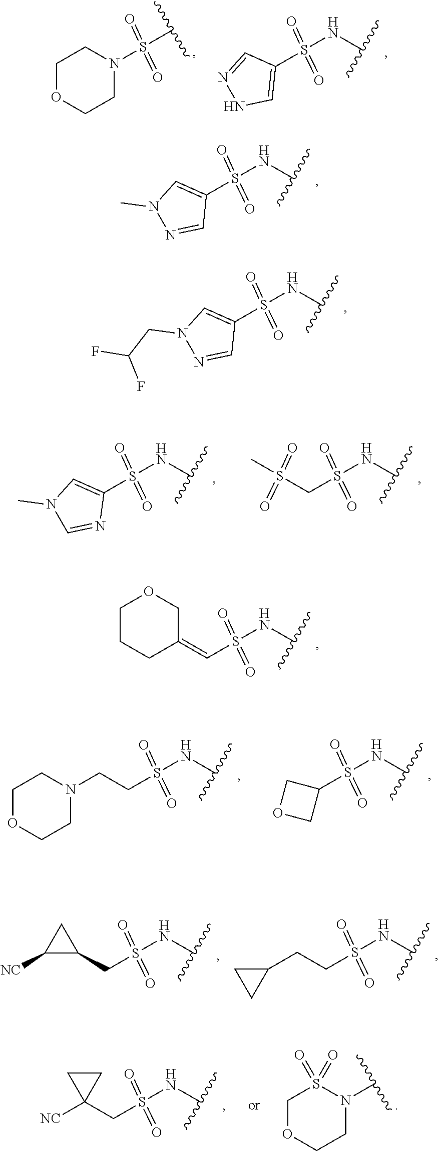

In some embodiments, R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHC(O)—C(O)CH₃, —NHSO₂N(CH₃)₂, —NHSO₂NH(CH₂CH₃), —SO₂CH₂CH₂OH, —SO₂NHCH₂CH₂OH,

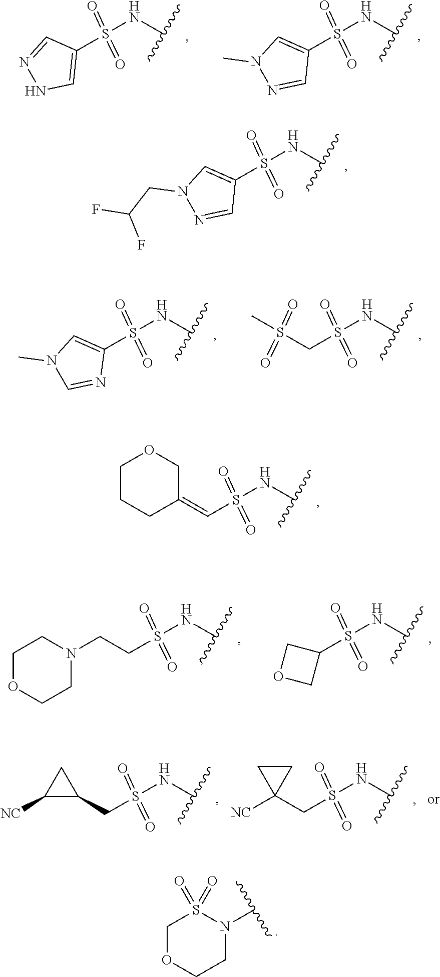

In some embodiments, R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHC(O)—C(O)CH₃, —SO₂CH₂CH₂OH, —SO₂NHCH₂CH₂OH, or

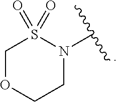 [SMILES: *N1CCOCS1(=O)=O]

In some embodiments, R³ is —NHSO₂CH₂CH₃, —NHSO₂CH₂CH₂OH, —SO₂CH₂CH₃, or —SO₂CH₂CH₂OH. In some embodiments, R³ is —NHSO₂CH₂CH₃ or —NHSO₂CH₂CH₂OH. In some embodiments, R³ is —NHSO₂CH₂CH₂OH. In some embodiments, R³ is —NHSO₂CH₂CH₃. In some embodiments, R³ is —SO₂CH₂CH₃ or —SO₂CH₂CH₂OH. In some embodiments, R³ is —SO₂CH₂CH₃. In some embodiments, R³ is —SO₂CH₂CH₂OH.

Any combination of the groups described above for the various variables is contemplated herein. Throughout the specification, groups and substituents thereof are chosen by one skilled in the field to provide stable moieties and compounds.

Provided in some embodiments herein is a compound having a structure provided in Table 1, a stereoisomer thereof, or a pharmaceutically acceptable salt, solvate, or isotopologue of the compound or the stereoisomer.

TABLE 1

| | | Mass Spec | HRMS |
|---|---|---|---|
| Cpd No. | Structure | M + H/1 | M + H/1 |
| 1 | 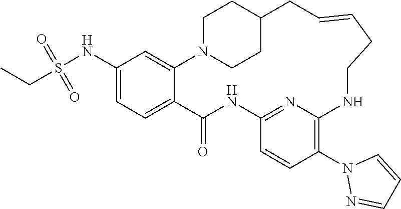 [SMILES: CCS(=O)(=O)Nc1ccc2c(c1)N1CCC(C/C=C/CCNc3nc(ccc3-n3cccn3)NC2=O)CC1] | 536.2 | 536.2428 |
| 2 | 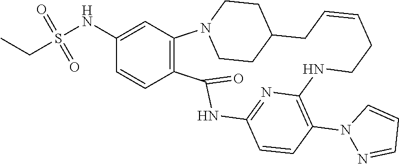 [SMILES: CCS(=O)(=O)Nc1ccc2c(c1)N1CCC(C/C=C\CCNc3nc(ccc3-n3cccn3)NC2=O)CC1] | 536.2 | 536.2422 |
| 3 | 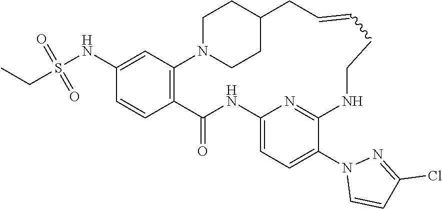 [SMILES: CCS(=O)(=O)Nc1ccc2c(c1)N1CCC(CC=CCCNc3nc(ccc3-n3ccc(Cl)n3)NC2=O)CC1] | 570.2 | |
| 4 | 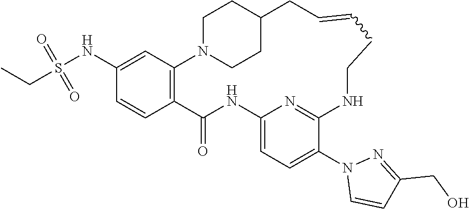 [SMILES: CCS(=O)(=O)Nc1ccc2c(c1)N1CCC(CC=CCCNc3nc(ccc3-n3ccc(CO)n3)NC2=O)CC1] | 566.3 | |
| 5 | 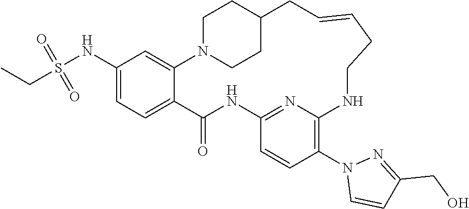 [SMILES: CCS(=O)(=O)Nc1ccc2c(c1)N1CCC(C/C=C/CCNc3nc(ccc3-n3ccc(CO)n3)NC2=O)CC1] | 566.3 | 566.2540 |
| 6 | 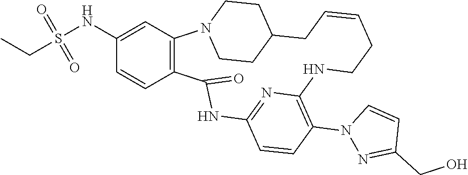 [SMILES: CCS(=O)(=O)Nc1ccc2c(c1)N1CCC(C/C=C\CCNc3nc(ccc3-n3ccc(CO)n3)NC2=O)CC1] | 566.3 | 566.2528 |
| 7 | 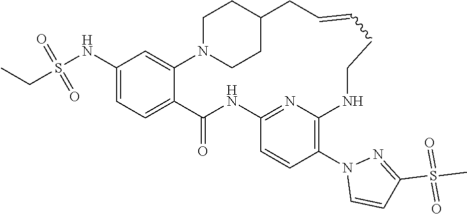 [SMILES: CCS(=O)(=O)Nc1ccc2c(c1)N1CCC(CC=CCCNc3nc(ccc3-n3ccc(S(C)(=O)=O)n3)NC2=O)CC1] | 614.2 | 614.2199 |
| 8 | 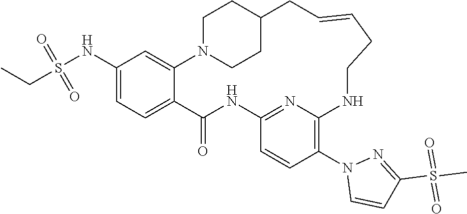 [SMILES: CCS(=O)(=O)Nc1ccc2c(c1)N1CCC(C/C=C/CCNc3nc(ccc3-n3ccc(S(C)(=O)=O)n3)NC2=O)CC1] | 614.2 | 614.2199 |
| 9 | 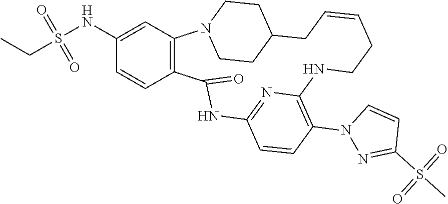 [SMILES: CCS(=O)(=O)Nc1ccc2c(c1)N1CCC(C/C=C\CCNc3nc(ccc3-n3ccc(S(C)(=O)=O)n3)NC2=O)CC1] | 614.2 | 614.2199 |
| 10 | 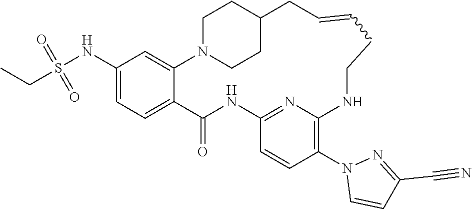 [SMILES: CCS(=O)(=O)Nc1ccc2c(c1)N1CCC(CC=CCCNc3nc(ccc3-n3ccc(C#N)n3)NC2=O)CC1] | 561.3 | 561.2374 |
| 11 | 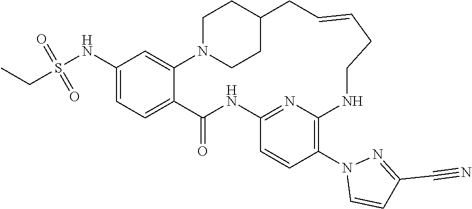 [SMILES: CCS(=O)(=O)Nc1ccc2c(c1)N1CCC(C/C=C/CCNc3nc(ccc3-n3ccc(C#N)n3)NC2=O)CC1] | 561.3 | 561.2389 |
| 12 | 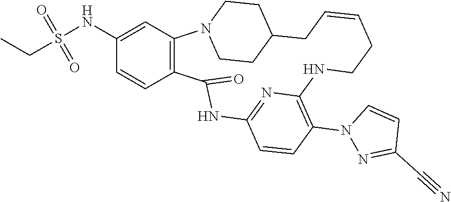 [SMILES: CCS(=O)(=O)Nc1ccc2c(c1)N1CCC(C/C=C\CCNc3nc(ccc3-n3ccc(C#N)n3)NC2=O)CC1] | 561.3 | 561.2395 |
| 13 | 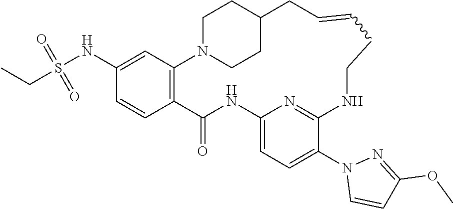 [SMILES: CCS(=O)(=O)Nc1ccc2c(c1)N1CCC(CC=CCCNc3nc(ccc3-n3ccc(OC)n3)NC2=O)CC1] | 566.4 | 566.2530 |
| 14 | 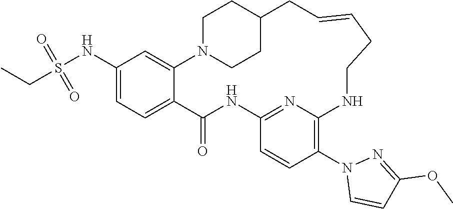 [SMILES: CCS(=O)(=O)Nc1ccc2c(c1)N1CCC(C/C=C/CCNc3nc(ccc3-n3ccc(OC)n3)NC2=O)CC1] | 566.4 | 566.2529 |
| 15 | 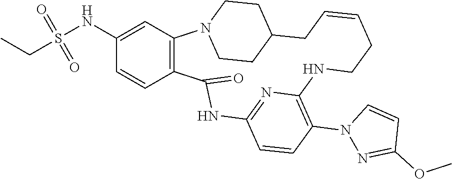 [SMILES: CCS(=O)(=O)Nc1ccc2c(c1)N1CCC(C/C=C\CCNc3nc(ccc3-n3ccc(OC)n3)NC2=O)CC1] | 566.3 | 566.2523 |
| 16 | 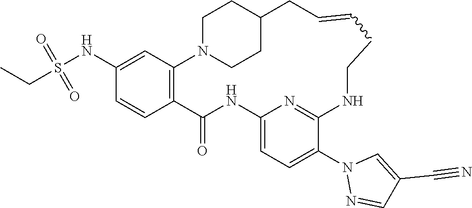 [SMILES: CCS(=O)(=O)Nc1ccc2c(c1)N1CCC(CC=CCCNc3nc(ccc3-n3cc(C#N)cn3)NC2=O)CC1] | 561.3 | 561.2386 |
| 17 | 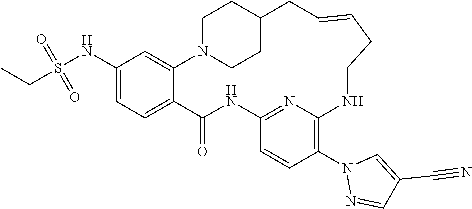 [SMILES: CCS(=O)(=O)Nc1ccc2c(c1)N1CCC(C/C=C/CCNc3nc(ccc3-n3cc(C#N)cn3)NC2=O)CC1] | 561.3 | 561.2383 |
| 18 | 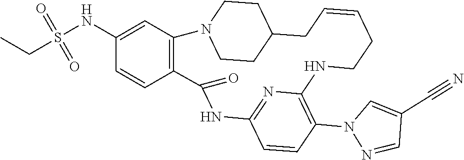 [SMILES: CCS(=O)(=O)Nc1ccc2c(c1)N1CCC(C/C=C\CCNc3nc(ccc3-n3cc(C#N)cn3)NC2=O)CC1] | 561.3 | 561.2377 |
| 19 | 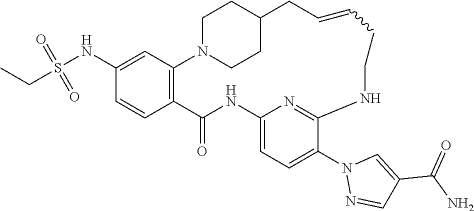 [SMILES: CCS(=O)(=O)Nc1ccc2c(c1)N1CCC(CC=CCCNc3nc(ccc3-n3cc(C(N)=O)cn3)NC2=O)CC1] | 579.3 | 579.2489 |
| 20 | 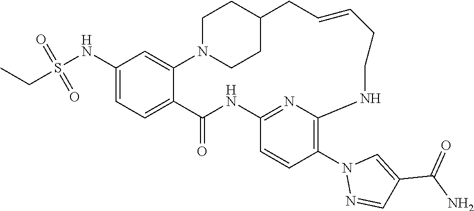 [SMILES: CCS(=O)(=O)Nc1ccc2c(c1)N1CCC(C/C=C/CCNc3nc(ccc3-n3cc(C(N)=O)cn3)NC2=O)CC1] | 579.3 | 579.2481 |
| 21 | 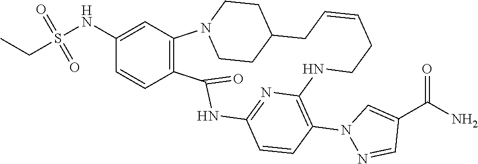 [SMILES: CCS(=O)(=O)Nc1ccc2c(c1)N1CCC(C/C=C\CCNc3nc(ccc3-n3cc(C(N)=O)cn3)NC2=O)CC1] | 579.3 | |
| 22 | 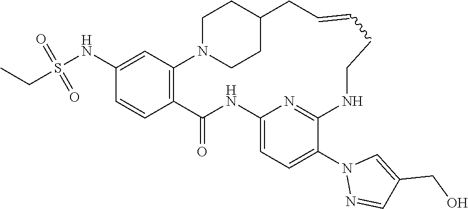 [SMILES: CCS(=O)(=O)Nc1ccc2c(c1)N1CCC(CC=CCCNc3nc(ccc3-n3cc(CO)cn3)NC2=O)CC1] | 556.2 | 566.2530 |
| 23 | 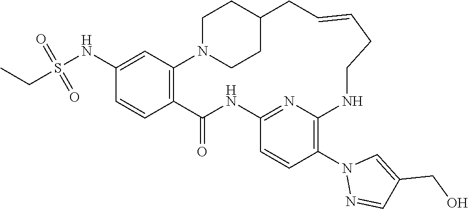 [SMILES: CCS(=O)(=O)Nc1ccc2c(c1)N1CCC(C/C=C/CCNc3nc(ccc3-n3cc(CO)cn3)NC2=O)CC1] | 566.3 | 566.2532 |
| 24 | 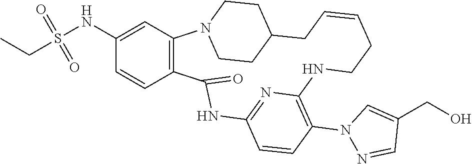 [SMILES: CCS(=O)(=O)Nc1ccc2c(c1)N1CCC(C/C=C\CCNc3nc(ccc3-n3cc(CO)cn3)NC2=O)CC1] | 566.3 | 566.2522 |
| 25 | 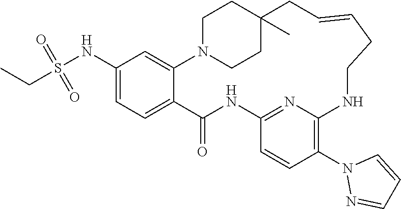 [SMILES: CCS(=O)(=O)Nc1ccc2c(c1)N1CCC(C)(C/C=C/CCNc3nc(ccc3-n3cccn3)NC2=O)CC1] | 550.3 | 550.2590 |
| 26 | 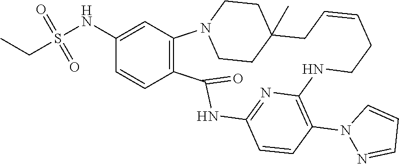 [SMILES: CCS(=O)(=O)Nc1ccc2c(c1)N1CCC(C)(C/C=C\CCNc3nc(ccc3-n3cccn3)NC2=O)CC1] | 550.4 | 550.2586 |
| 27 | 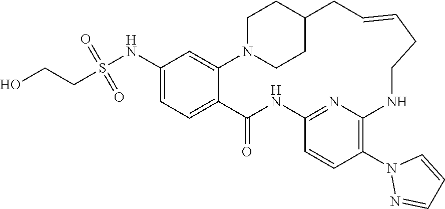 [SMILES: O=C1Nc2ccc(-n3cccn3)c(n2)NCC/C=C/CC2CCN(CC2)c2cc(NS(=O)(=O)CCO)ccc21] | 552.3 | 552.2376 |
| 28 | 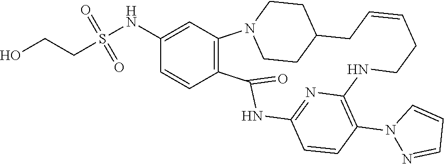 [SMILES: O=C1Nc2ccc(-n3cccn3)c(n2)NCC/C=C\CC2CCN(CC2)c2cc(NS(=O)(=O)CCO)ccc21] | 552.3 | 552.2375 |
| 29 | 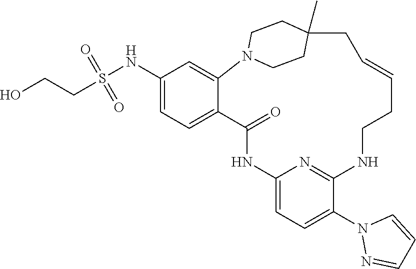 [SMILES: CC12C/C=C/CCNc3nc(ccc3-n3cccn3)NC(=O)c3ccc(NS(=O)(=O)CCO)cc3N(CC1)CC2] | 566.3 | 566.2532 |
| 30 | 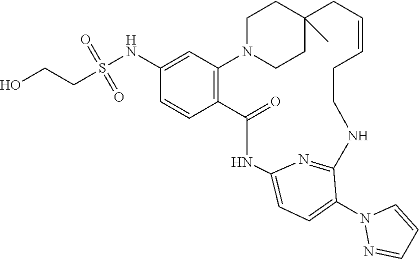 [SMILES: CC12C/C=C\CCNc3nc(ccc3-n3cccn3)NC(=O)c3ccc(NS(=O)(=O)CCO)cc3N(CC1)CC2] | 566.3 | 566.2530 |
| 31 | 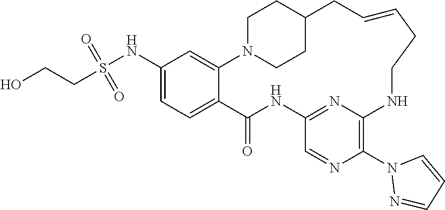 [SMILES: O=C1Nc2cnc(-n3cccn3)c(n2)NCC/C=C/CC2CCN(CC2)c2cc(NS(=O)(=O)CCO)ccc21] | 553.3 | 553.2326 |
| 32 | 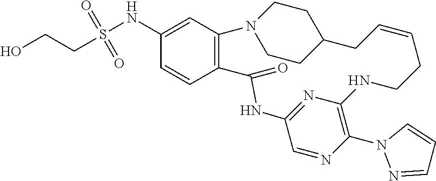 [SMILES: O=C1Nc2cnc(-n3cccn3)c(n2)NCC/C=C\CC2CCN(CC2)c2cc(NS(=O)(=O)CCO)ccc21] | 553.1 | 553.2334 |
| 33 | 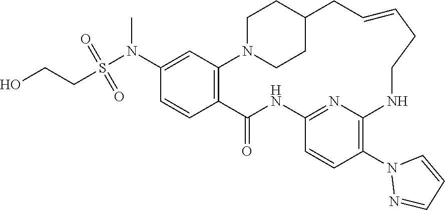 [SMILES: CN(c1ccc2c(c1)N1CCC(C/C=C/CCNc3nc(ccc3-n3cccn3)NC2=O)CC1)S(=O)(=O)CCO] | 566.2 | 566.2540 |
| 34 | 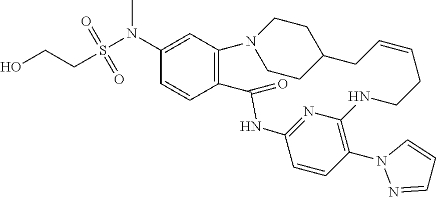 [SMILES: CN(c1ccc2c(c1)N1CCC(C/C=C\CCNc3nc(ccc3-n3cccn3)NC2=O)CC1)S(=O)(=O)CCO] | 566.3 | 566.2536 |
| 35 | 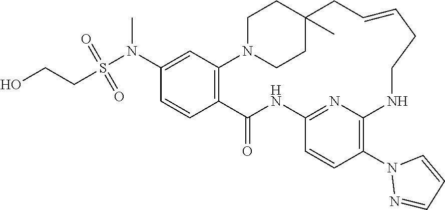 [SMILES: CN(c1ccc2c(c1)N1CCC(C)(C/C=C/CCNc3nc(ccc3-n3cccn3)NC2=O)CC1)S(=O)(=O)CCO] | 580.3 | 580.2692 |
| 36 | 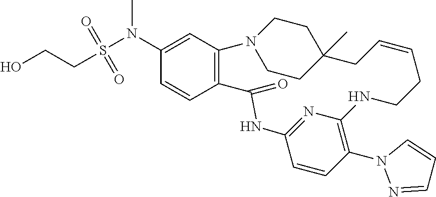 [SMILES: CN(c1ccc2c(c1)N1CCC(C)(C/C=C\CCNc3nc(ccc3-n3cccn3)NC2=O)CC1)S(=O)(=O)CCO] | 580.3 | 580.2686 |
| 37 | 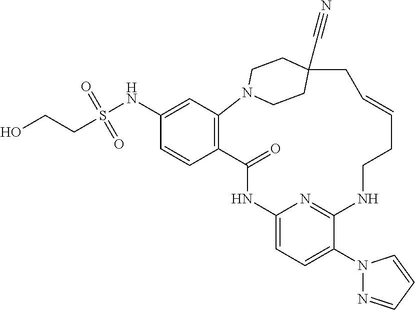 [SMILES: N#CC12C/C=C/CCNc3nc(ccc3-n3cccn3)NC(=O)c3ccc(NS(=O)(=O)CCO)cc3N(CC1)CC2] | 577.2 | 577.2331 |
| 38 | 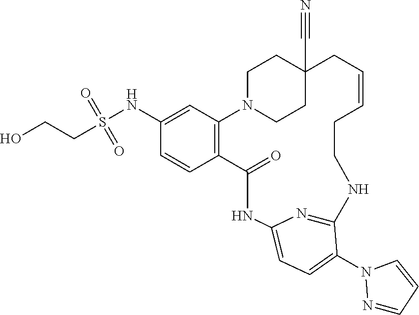 [SMILES: N#CC12C/C=C\CCNc3nc(ccc3-n3cccn3)NC(=O)c3ccc(NS(=O)(=O)CCO)cc3N(CC1)CC2] | 577.2 | 577.2331 |
| 39 | 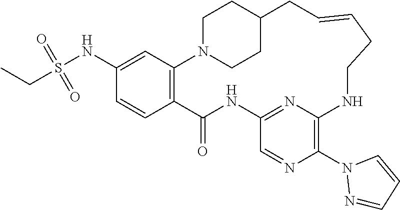 [SMILES: CCS(=O)(=O)Nc1ccc2c(c1)N1CCC(C/C=C/CCNc3nc(cnc3-n3cccn3)NC2=O)CC1] | 537.4 | 537.2379 |
| 40 | 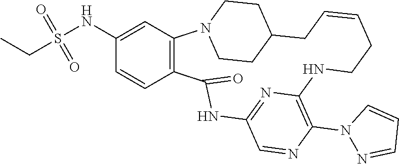 [SMILES: CCS(=O)(=O)Nc1ccc2c(c1)N1CCC(C/C=C\CCNc3nc(cnc3-n3cccn3)NC2=O)CC1] | 537.3 | 537.2380 |
| 41 | 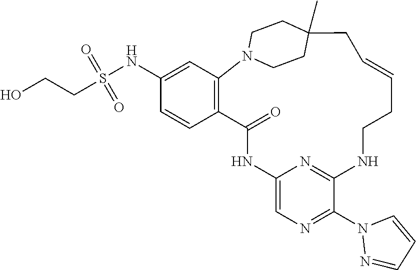 [SMILES: CC12C/C=C/CCNc3nc(cnc3-n3cccn3)NC(=O)c3ccc(NS(=O)(=O)CCO)cc3N(CC1)CC2] | 567.2 | 567.2489 |
| 42 | 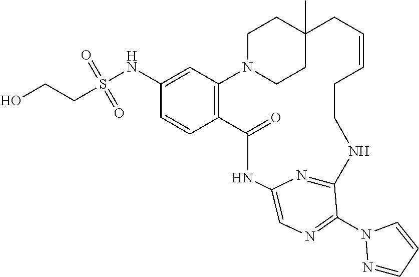 [SMILES: CC12C/C=C\CCNc3nc(cnc3-n3cccn3)NC(=O)c3ccc(NS(=O)(=O)CCO)cc3N(CC1)CC2] | 567.2 | 567.2483 |
| 43 | 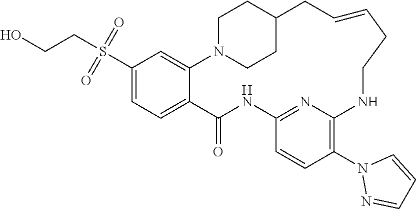 [SMILES: O=C1Nc2ccc(-n3cccn3)c(n2)NCC/C=C/CC2CCN(CC2)c2cc(S(=O)(=O)CCO)ccc21] | 537.3 | 537.2280 |
| 44 | 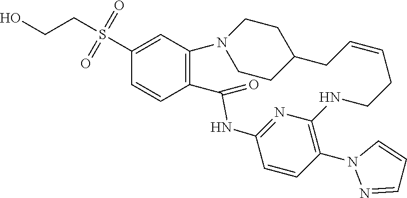 [SMILES: O=C1Nc2ccc(-n3cccn3)c(n2)NCC/C=C\CC2CCN(CC2)c2cc(S(=O)(=O)CCO)ccc21] | 537.2 | 537.2265 |
| 45 | 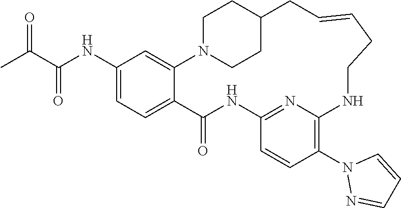 [SMILES: CC(=O)C(=O)Nc1ccc2c(c1)N1CCC(C/C=C/CCNc3nc(ccc3-n3cccn3)NC2=O)CC1] | 514.3 | 514.2554 |
| 46 | 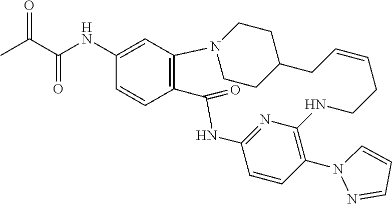 [SMILES: CC(=O)C(=O)Nc1ccc2c(c1)N1CCC(C/C=C\CCNc3nc(ccc3-n3cccn3)NC2=O)CC1] | 514.2 | 514.2566 |
| 47 | 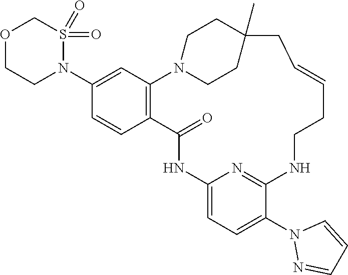 [SMILES: CC12C/C=C/CCNc3nc(ccc3-n3cccn3)NC(=O)c3ccc(N4CCOCS4(=O)=O)cc3N(CC1)CC2] | 578.2 | 578.2533 |
| 48 | 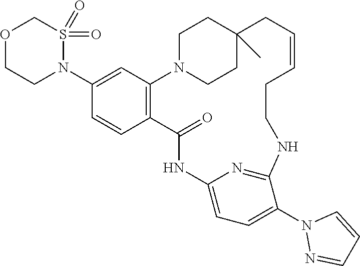 [SMILES: CC12C/C=C\CCNc3nc(ccc3-n3cccn3)NC(=O)c3ccc(N4CCOCS4(=O)=O)cc3N(CC1)CC2] | 578.3 | 578.2534 |
| 49 | 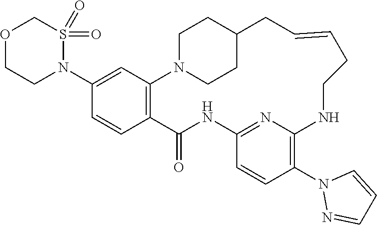 [SMILES: O=C1Nc2ccc(-n3cccn3)c(n2)NCC/C=C/CC2CCN(CC2)c2cc(N3CCOCS3(=O)=O)ccc21] | 564.2 | 564.2376 |
| 50 | 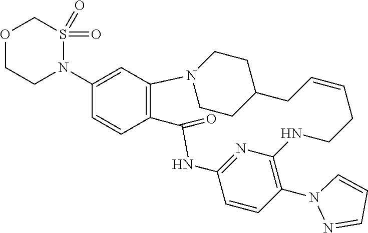 [SMILES: O=C1Nc2ccc(-n3cccn3)c(n2)NCC/C=C\CC2CCN(CC2)c2cc(N3CCOCS3(=O)=O)ccc21] | 564.2 | 564.2375 |
| 51 | 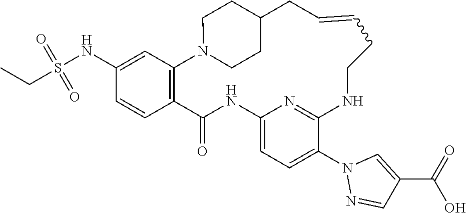 [SMILES: CCS(=O)(=O)Nc1ccc2c(c1)N1CCC(CC=CCCNc3nc(ccc3-n3cc(C(=O)O)cn3)NC2=O)CC1] | 580.2 | 580.2326 |
| 52 | 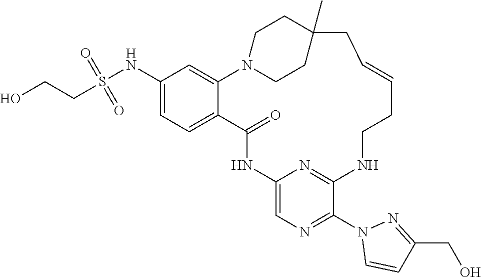 [SMILES: CC12C/C=C/CCNc3nc(cnc3-n3ccc(CO)n3)NC(=O)c3ccc(NS(=O)(=O)CCO)cc3N(CC1)CC2] | 597.3 | 597.2578 |
| 53 | 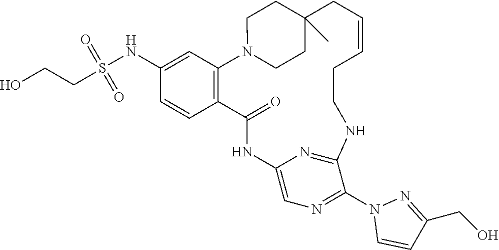 [SMILES: CC12C/C=C\CCNc3nc(cnc3-n3ccc(CO)n3)NC(=O)c3ccc(NS(=O)(=O)CCO)cc3N(CC1)CC2] | 597.2 | 597.2580 |
| 54 | 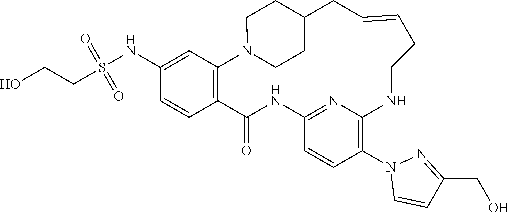 [SMILES: O=C1Nc2ccc(-n3ccc(CO)n3)c(n2)NCC/C=C/CC2CCN(CC2)c2cc(NS(=O)(=O)CCO)ccc21] | 582.3 | 582.2478 |
| 55 | 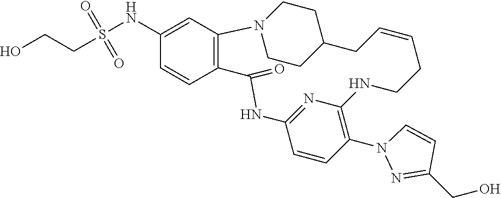 [SMILES: O=C1Nc2ccc(-n3ccc(CO)n3)c(n2)NCC/C=C\CC2CCN(CC2)c2cc(NS(=O)(=O)CCO)ccc21] | 582.3 | 582.2475 |
| 56 | 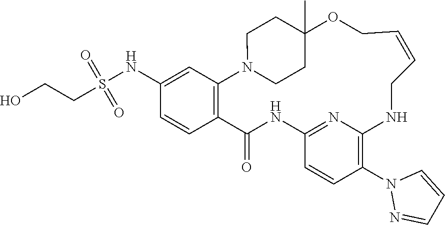 [SMILES: CC12CCN(CC1)c1cc(NS(=O)(=O)CCO)ccc1C(=O)Nc1ccc(-n3cccn3)c(n1)NC/C=C\CO2] | 568.3 | 568.2339 |
| 57 | 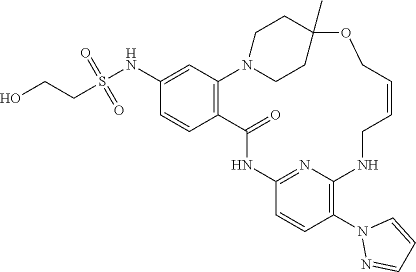 [SMILES: CC12CCN(CC1)c1cc(NS(=O)(=O)CCO)ccc1C(=O)Nc1ccc(-n3cccn3)c(n1)NC/C=C\CO2] | 568.3 | 568.2343 |
| 58 | 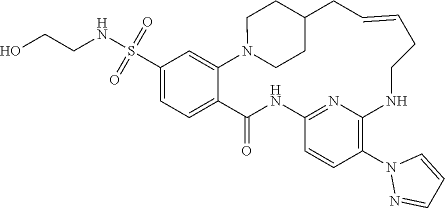 [SMILES: O=C1Nc2ccc(-n3cccn3)c(n2)NCC/C=C/CC2CCN(CC2)c2cc(S(=O)(=O)NCCO)ccc21] | 552.3 | 552.2374 |
| 59 | 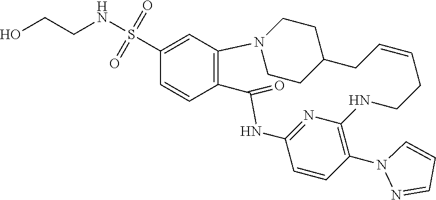 [SMILES: O=C1Nc2ccc(-n3cccn3)c(n2)NCC/C=C\CC2CCN(CC2)c2cc(S(=O)(=O)NCCO)ccc21] | 552.3 | 552.2365 |
| 60 | 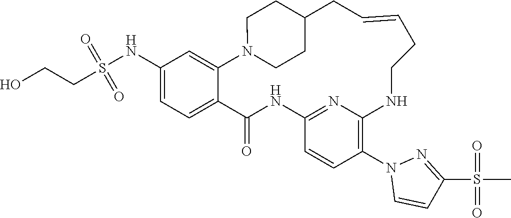 [SMILES: CS(=O)(=O)c1ccn(-c2ccc3nc2NCC/C=C/CC2CCN(CC2)c2cc(NS(=O)(=O)CCO)ccc2C(=O)N3)n1] | 630.3 | 630.2146 |
| 61 | 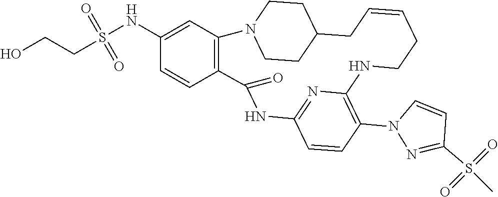 [SMILES: CS(=O)(=O)c1ccn(-c2ccc3nc2NCC/C=C\CC2CCN(CC2)c2cc(NS(=O)(=O)CCO)ccc2C(=O)N3)n1] | 630.3 | 630.2150 |
| 62 | 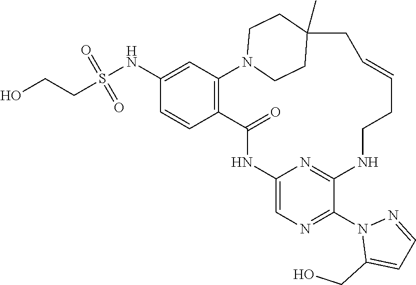 [SMILES: CC12C/C=C/CCNc3nc(cnc3-n3nccc3CO)NC(=O)c3ccc(NS(=O)(=O)CCO)cc3N(CC1)CC2] | 597.3 | 597.2603 |
| 63 | 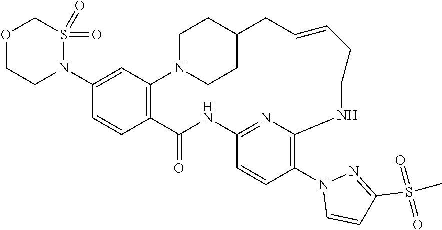 [SMILES: CS(=O)(=O)c1ccn(-c2ccc3nc2NCC/C=C/CC2CCN(CC2)c2cc(N4CCOCS4(=O)=O)ccc2C(=O)N3)n1] | 642.3 | 642.2156 |
| 64 | 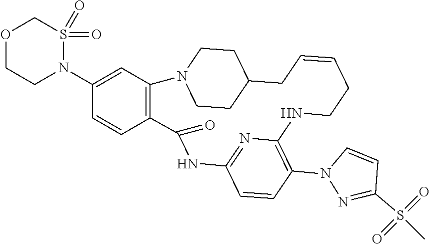 [SMILES: CS(=O)(=O)c1ccn(-c2ccc3nc2NCC/C=C\CC2CCN(CC2)c2cc(N4CCOCS4(=O)=O)ccc2C(=O)N3)n1] | 642.3 | 642.2161 |
| 65 | 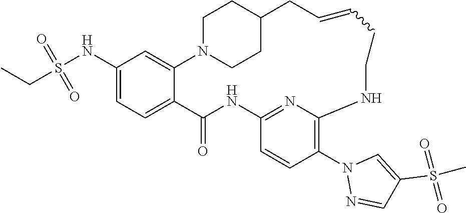 [SMILES: CCS(=O)(=O)Nc1ccc2c(c1)N1CCC(CC=CCCNc3nc(ccc3-n3cc(S(C)(=O)=O)cn3)NC2=O)CC1] | 614.2 | 614.2192 |
| 66 | 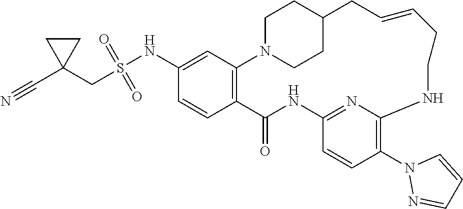 [SMILES: N#CC1(CS(=O)(=O)Nc2ccc3c(c2)N2CCC(C/C=C/CCNc4nc(ccc4-n4cccn4)NC3=O)CC2)CC1] | 587.2 | 587.2541 |
| 67 | 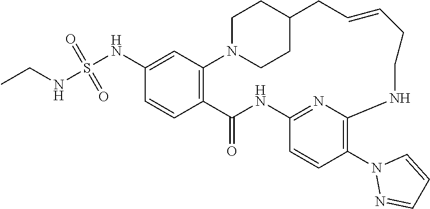 [SMILES: CCNS(=O)(=O)Nc1ccc2c(c1)N1CCC(C/C=C/CCNc3nc(ccc3-n3cccn3)NC2=O)CC1] | 551.2 | 551.2546 |
| 68 | 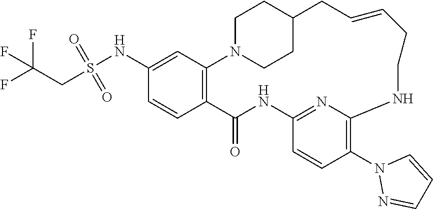 [SMILES: O=C1Nc2ccc(-n3cccn3)c(n2)NCC/C=C/CC2CCN(CC2)c2cc(NS(=O)(=O)CC(F)(F)F)ccc21] | 590.2 | 590.2167 |
| 69 | 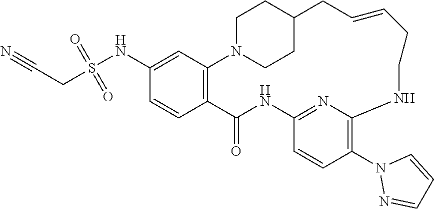 [SMILES: N#CCS(=O)(=O)Nc1ccc2c(c1)N1CCC(C/C=C/CCNc3nc(ccc3-n3cccn3)NC2=O)CC1] | 547.2 | 547.2227 |
| 70 | 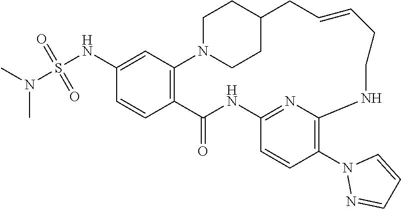 [SMILES: CN(C)S(=O)(=O)Nc1ccc2c(c1)N1CCC(C/C=C/CCNc3nc(ccc3-n3cccn3)NC2=O)CC1] | 551.2 | 551.2539 |
| 71 | 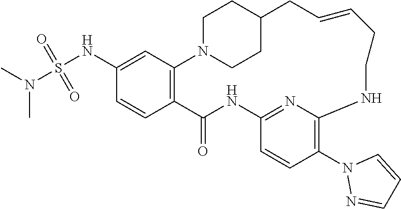 [SMILES: CN(C)S(=O)(=O)Nc1ccc2c(c1)N1CCC(C/C=C/CCNc3nc(ccc3-n3cccn3)NC2=O)CC1] | 563.2 | 563.2533 |
| 72 | 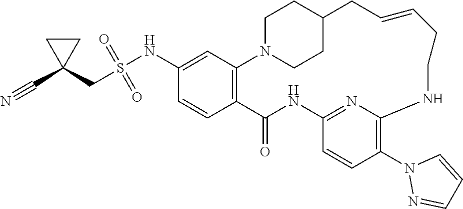 | 587.2 | 587.2531 |
| 73 | 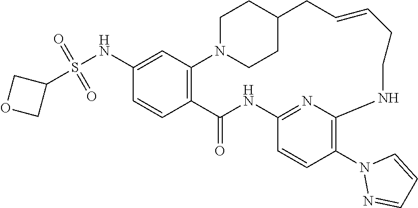 [SMILES: O=C1Nc2ccc(-n3cccn3)c(n2)NCC/C=C/CC2CCN(CC2)c2cc(NS(=O)(=O)C3COC3)ccc21] | 564.2 | 564.2382 |
| 74 | 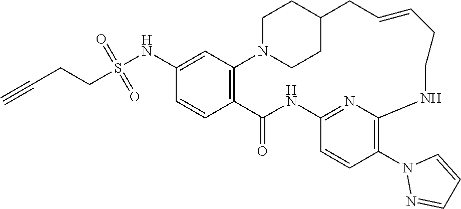 [SMILES: C#CCCS(=O)(=O)Nc1ccc2c(c1)N1CCC(C/C=C/CCNc3nc(ccc3-n3cccn3)NC2=O)CC1] | 560.2 | 560.2433 |
| 75 | 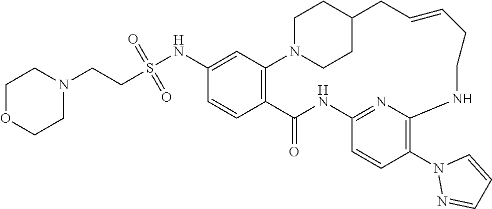 [SMILES: O=C1Nc2ccc(-n3cccn3)c(n2)NCC/C=C/CC2CCN(CC2)c2cc(NS(=O)(=O)CCN3CCOCC3)ccc21] | 621.3 | 621.2966 |
| 76 | 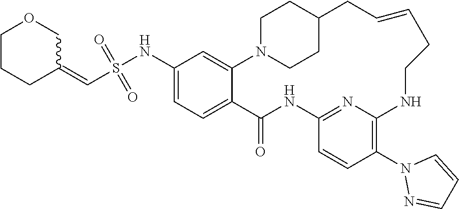 [SMILES: O=C1Nc2ccc(-n3cccn3)c(n2)NCC/C=C/CC2CCN(CC2)c2cc(NS(=O)(=O)C=C3CCCOC3)ccc21] | 604.3 | 604.2692 |
| 77 | 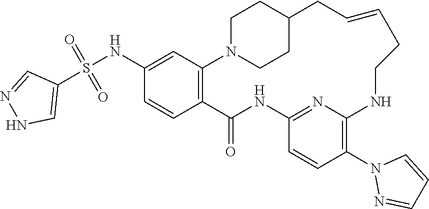 [SMILES: O=C1Nc2ccc(-n3cccn3)c(n2)NCC/C=C/CC2CCN(CC2)c2cc(NS(=O)(=O)c3cn[nH]c3)ccc21] | 574.2 | 574.2339 |
| 78 | 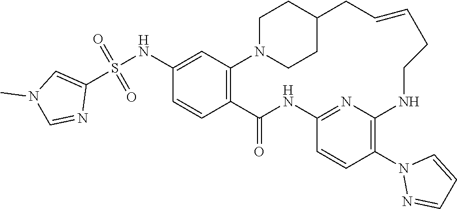 [SMILES: Cn1cnc(S(=O)(=O)Nc2ccc3c(c2)N2CCC(C/C=C/CCNc4nc(ccc4-n4cccn4)NC3=O)CC2)c1] | 588.2 | 588.2502 |
| 79 | 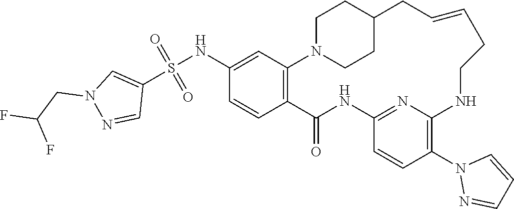 [SMILES: O=C1Nc2ccc(-n3cccn3)c(n2)NCC/C=C/CC2CCN(CC2)c2cc(NS(=O)(=O)c3cnn(CC(F)F)c3)ccc21] | 638.2 | 638.2452 |
| 80 | 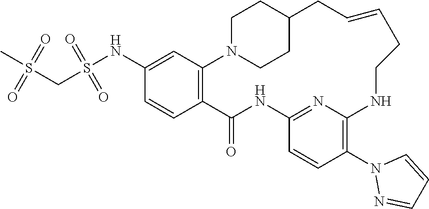 [SMILES: CS(=O)(=O)CS(=O)(=O)Nc1ccc2c(c1)N1CCC(C/C=C/CCNc3nc(ccc3-n3cccn3)NC2=O)CC1] | 600.2 | 600.2059 |
| 81 | 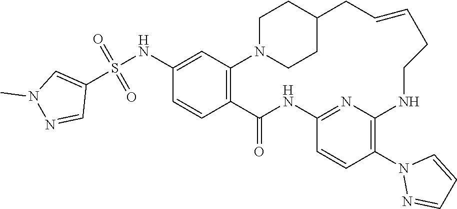 [SMILES: Cn1cc(S(=O)(=O)Nc2ccc3c(c2)N2CCC(C/C=C/CCNc4nc(ccc4-n4cccn4)NC3=O)CC2)cn1] | 588.2 | 588.2498 |
| 82 | 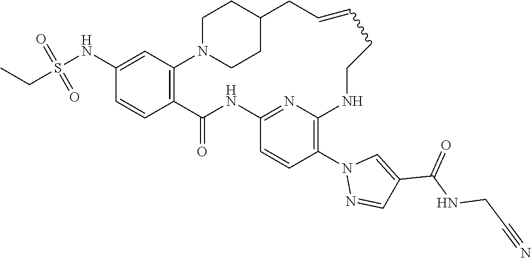 [SMILES: CCS(=O)(=O)Nc1ccc2c(c1)N1CCC(CC=CCCNc3nc(ccc3-n3cc(C(=O)NCC#N)cn3)NC2=O)CC1] | 618.2 | 618.2591 |
| 83 | 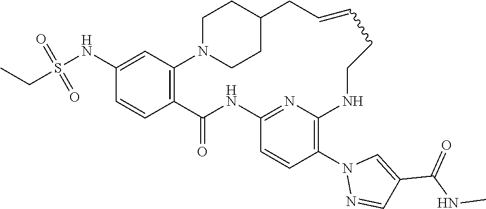 [SMILES: CCS(=O)(=O)Nc1ccc2c(c1)N1CCC(CC=CCCNc3nc(ccc3-n3cc(C(=O)NC)cn3)NC2=O)CC1] | 593.2 | 593.2642 |
| 84 | 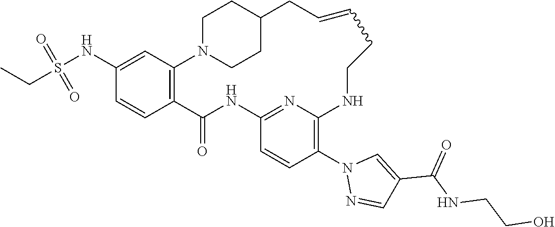 [SMILES: CCS(=O)(=O)Nc1ccc2c(c1)N1CCC(CC=CCCNc3nc(ccc3-n3cc(C(=O)NCCO)cn3)NC2=O)CC1] | 623.3 | 623.2738 |
| 85 | 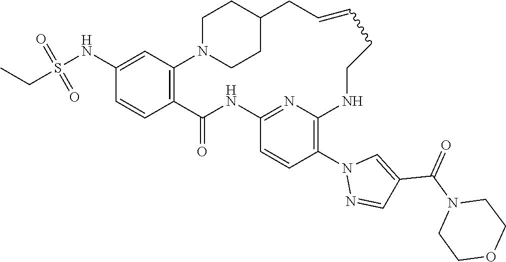 [SMILES: CCS(=O)(=O)Nc1ccc2c(c1)N1CCC(CC=CCCNc3nc(ccc3-n3cc(C(=O)N4CCOCC4)cn3)NC2=O)CC1] | 649.3 | 649.2912 |
| 86 | 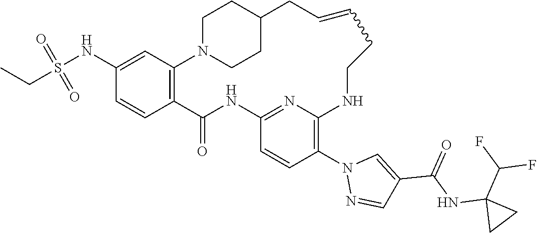 [SMILES: CCS(=O)(=O)Nc1ccc2c(c1)N1CCC(CC=CCCNc3nc(ccc3-n3cc(C(=O)NC4(C(F)F)CC4)cn3)NC2=O)CC1] | 669.3 | 669.2756 |
| 87 | 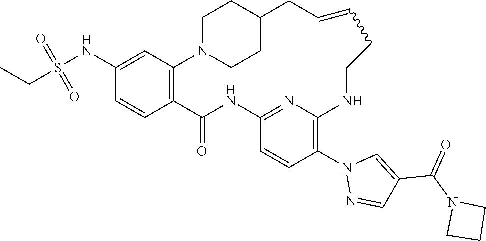 [SMILES: CCS(=O)(=O)Nc1ccc2c(c1)N1CCC(CC=CCCNc3nc(ccc3-n3cc(C(=O)N4CCC4)cn3)NC2=O)CC1] | 619.3 | 619.2804 |
| 88 | 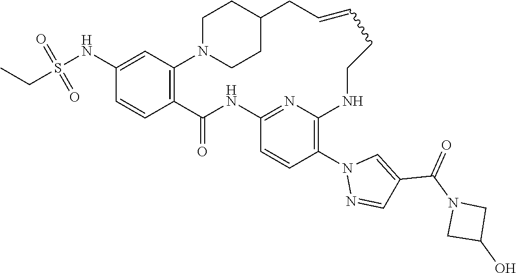 [SMILES: CCS(=O)(=O)Nc1ccc2c(c1)N1CCC(CC=CCCNc3nc(ccc3-n3cc(C(=O)N4CC(O)C4)cn3)NC2=O)CC1] | 635.3 | 635.2748 |
| 89 | 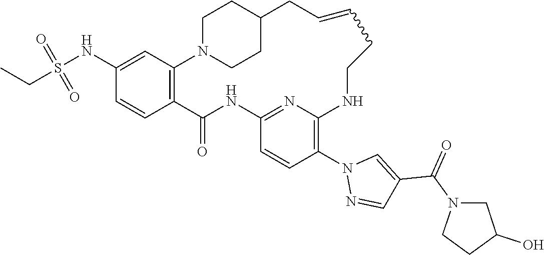 [SMILES: CCS(=O)(=O)Nc1ccc2c(c1)N1CCC(CC=CCCNc3nc(ccc3-n3cc(C(=O)N4CCC(O)C4)cn3)NC2=O)CC1] | 649.3 | 649.2911 |
| 90 | 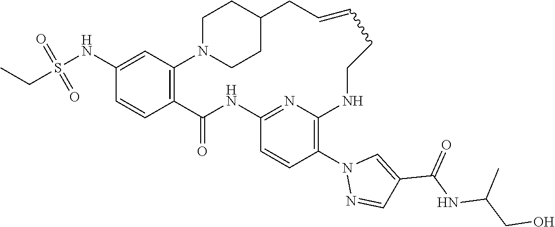 [SMILES: CCS(=O)(=O)Nc1ccc2c(c1)N1CCC(CC=CCCNc3nc(ccc3-n3cc(C(=O)NC(C)CO)cn3)NC2=O)CC1] | 637.3 | 637.2902 |
| 91 | 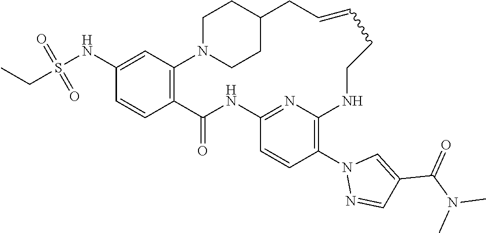 [SMILES: CCS(=O)(=O)Nc1ccc2c(c1)N1CCC(CC=CCCNc3nc(ccc3-n3cc(C(=O)N(C)C)cn3)NC2=O)CC1] | 607.3 | 607.2798 |
| 92 | 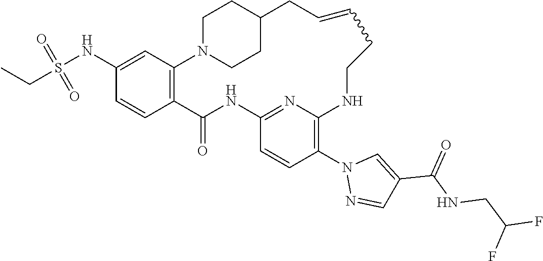 [SMILES: CCS(=O)(=O)Nc1ccc2c(c1)N1CCC(CC=CCCNc3nc(ccc3-n3cc(C(=O)NCC(F)F)cn3)NC2=O)CC1] | 643.3 | |
| 93 | 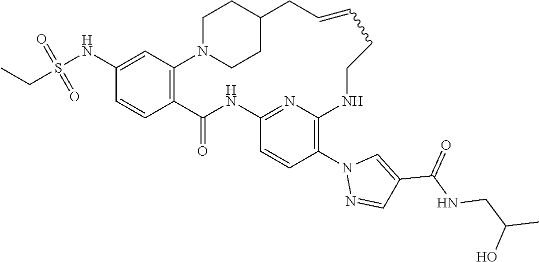 [SMILES: CCS(=O)(=O)Nc1ccc2c(c1)N1CCC(CC=CCCNc3nc(ccc3-n3cc(C(=O)NCC(C)O)cn3)NC2=O)CC1] | 637.3 | 637.2909 |
| 94 | 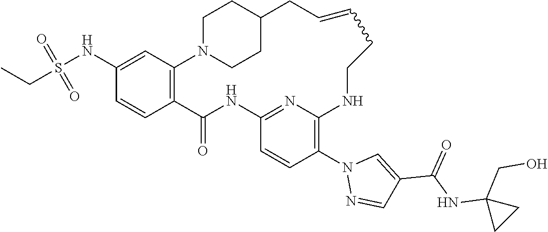 [SMILES: CCS(=O)(=O)Nc1ccc2c(c1)N1CCC(CC=CCCNc3nc(ccc3-n3cc(C(=O)NC4(CO)CC4)cn3)NC2=O)CC1] | 649.3 | 649.2895 |
| 95 | 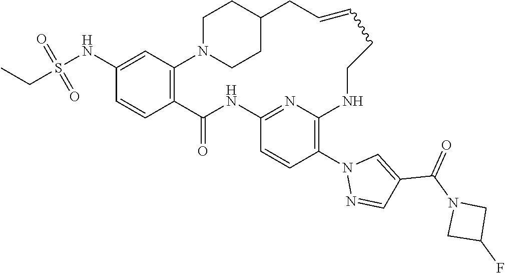 [SMILES: CCS(=O)(=O)Nc1ccc2c(c1)N1CCC(CC=CCCNc3nc(ccc3-n3cc(C(=O)N4CC(F)C4)cn3)NC2=O)CC1] | 637.3 | 637.2704 |
| 96 | 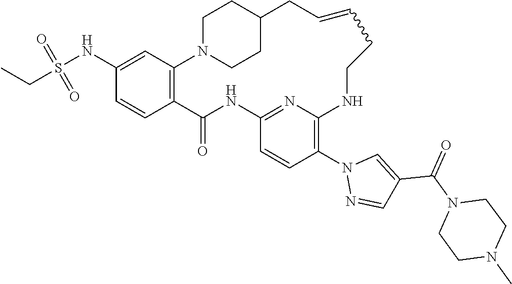 [SMILES: CCS(=O)(=O)Nc1ccc2c(c1)N1CCC(CC=CCCNc3nc(ccc3-n3cc(C(=O)N4CCN(C)CC4)cn3)NC2=O)CC1] | 662.3 | |
| 97 | 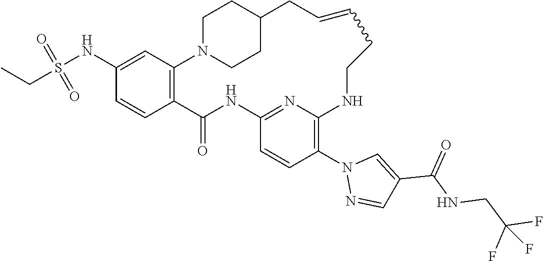 [SMILES: CCS(=O)(=O)Nc1ccc2c(c1)N1CCC(CC=CCCNc3nc(ccc3-n3cc(C(=O)NCC(F)(F)F)cn3)NC2=O)CC1] | 661.2 | 661.2514 |
| 98 | 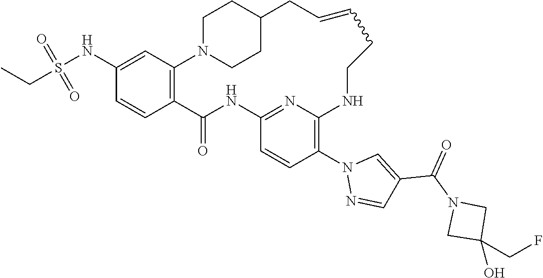 [SMILES: CCS(=O)(=O)Nc1ccc2c(c1)N1CCC(CC=CCCNc3nc(ccc3-n3cc(C(=O)N4CC(O)(CF)C4)cn3)NC2=O)CC1] | 667.3 | 667.2803 |
| 99 | 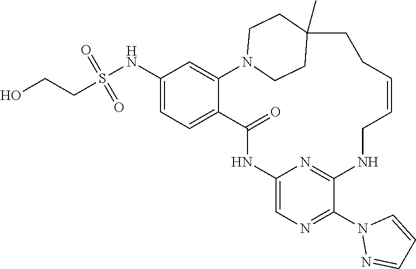 [SMILES: CC12CC/C=C\CNc3nc(cnc3-n3cccn3)NC(=O)c3ccc(NS(=O)(=O)CCO)cc3N(CC1)CC2] | 566.2 | 566.2544 |
| 100 | 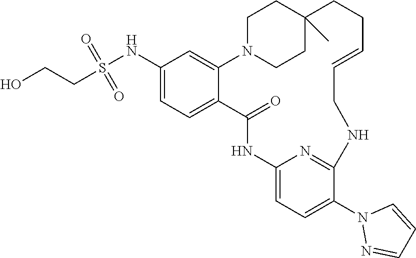 [SMILES: CC12CC/C=C/CNc3nc(ccc3-n3cccn3)NC(=O)c3ccc(NS(=O)(=O)CCO)cc3N(CC1)CC2] | 566.2 | 566.2527 |
| 101 | 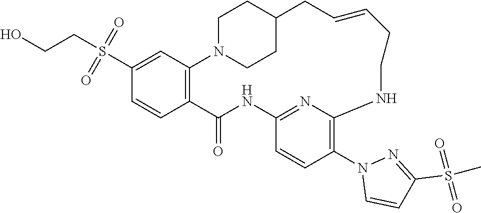 [SMILES: CS(=O)(=O)c1ccn(-c2ccc3nc2NCC/C=C/CC2CCN(CC2)c2cc(S(=O)(=O)CCO)ccc2C(=O)N3)n1] | 615.2 | 615.2048 |
| 102 | 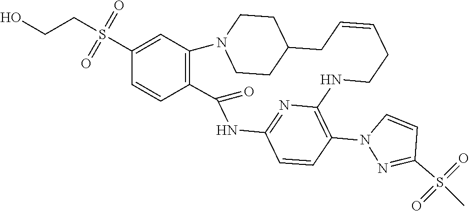 [SMILES: CS(=O)(=O)c1ccn(-c2ccc3nc2NCC/C=C\CC2CCN(CC2)c2cc(S(=O)(=O)CCO)ccc2C(=O)N3)n1] | 615.3 | 615.2054 |
| 103 | 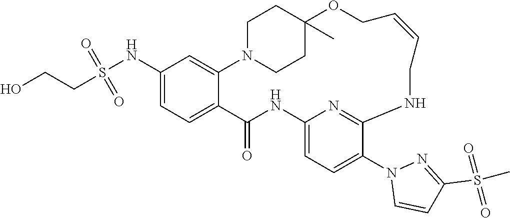 [SMILES: CC12CCN(CC1)c1cc(NS(=O)(=O)CCO)ccc1C(=O)Nc1ccc(-n3ccc(S(C)(=O)=O)n3)c(n1)NC/C=C\CO2] | 646.3 | 646.2095 |
| 104 | 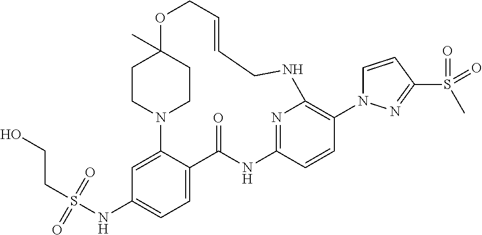 [SMILES: CC12CCN(CC1)c1cc(NS(=O)(=O)CCO)ccc1C(=O)Nc1ccc(-n3ccc(S(C)(=O)=O)n3)c(n1)NC/C=C/CO2] | 646.2 | 646.2097 |
| 105 | 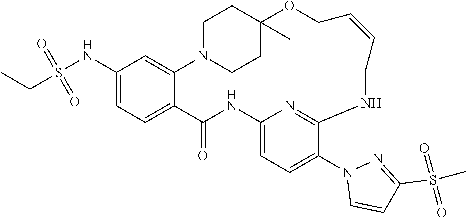 [SMILES: CCS(=O)(=O)Nc1ccc2c(c1)N1CCC(C)(CC1)OC/C=C\CNc1nc(ccc1-n1ccc(S(C)(=O)=O)n1)NC2=O] | 630.2 | 630.2154 |
| 106 | 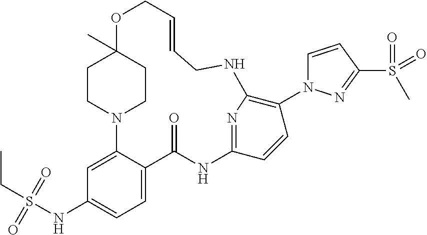 [SMILES: CCS(=O)(=O)Nc1ccc2c(c1)N1CCC(C)(CC1)OC/C=C/CNc1nc(ccc1-n1ccc(S(C)(=O)=O)n1)NC2=O] | 630.3 | 630.2147 |
| 107 | 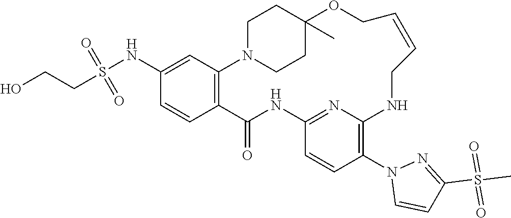 [SMILES: CC12CCN(CC1)c1cc(NS(=O)(=O)CCO)ccc1C(=O)Nc1ccc(-n3ccc(S(C)(=O)=O)n3)c(n1)NC/C=C\CO2] | 631.2 | 631.1998 |
| 108 | 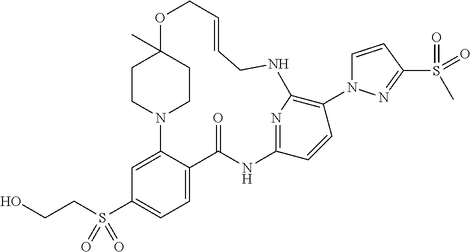 [SMILES: CC12CCN(CC1)c1cc(S(=O)(=O)CCO)ccc1C(=O)Nc1ccc(-n3ccc(S(C)(=O)=O)n3)c(n1)NC/C=C/CO2] | 631.1 | 631.1994 |
| 109 | 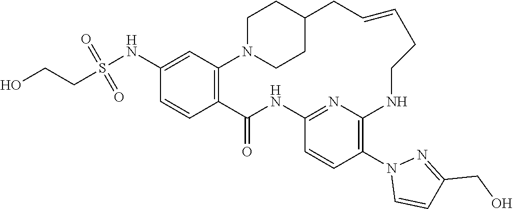 [SMILES: O=C1Nc2ccc(-n3ccc(CO)n3)c(n2)NCC/C=C/CC2CCN(CC2)c2cc(NS(=O)(=O)CCO)ccc21] | 582.2 | 582.2490 |
| 110 | 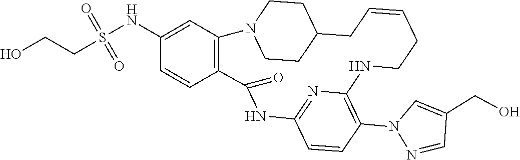 [SMILES: O=C1Nc2ccc(-n3cc(CO)cn3)c(n2)NCC/C=C\CC2CCN(CC2)c2cc(NS(=O)(=O)CCO)ccc21] | 582.2 | 582.2482 |
| 111 | 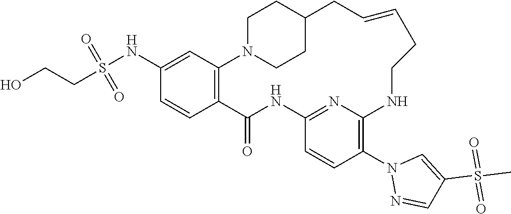 [SMILES: CS(=O)(=O)c1cnn(-c2ccc3nc2NCC/C=C/CC2CCN(CC2)c2cc(NS(=O)(=O)CCO)ccc2C(=O)N3)c1] | 630.2 | 630.2149 |
| 112 | 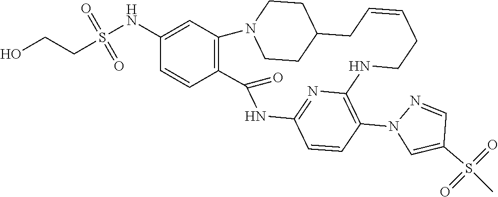 [SMILES: CS(=O)(=O)c1cnn(-c2ccc3nc2NCC/C=C\CC2CCN(CC2)c2cc(NS(=O)(=O)CCO)ccc2C(=O)N3)c1] | 630.2 | |
| 113 | 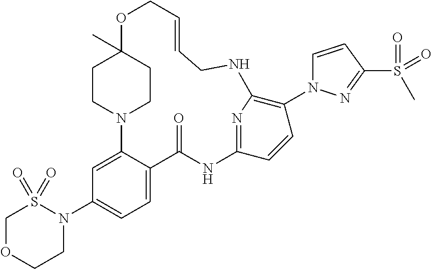 [SMILES: CC12CCN(CC1)c1cc(N3CCOCS3(=O)=O)ccc1C(=O)Nc1ccc(-n3ccc(S(C)(=O)=O)n3)c(n1)NC/C=C/CO2] | 658.2 | 658.2097 |
| 114 | 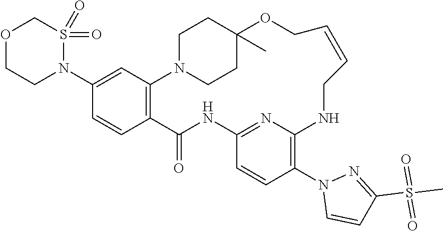 [SMILES: CC12CCN(CC1)c1cc(N3CCOCS3(=O)=O)ccc1C(=O)Nc1ccc(-n3ccc(S(C)(=O)=O)n3)c(n1)NC/C=C\CO2] | 658.2 | 658.2103 |
| 115 | 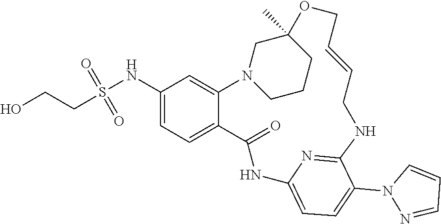 [SMILES: C[C@]12CCCN(C1)c1cc(NS(=O)(=O)CCO)ccc1C(=O)Nc1ccc(-n3cccn3)c(n1)NC/C=C/CO2] | 568.3 | 568.2335 |
| 116 | 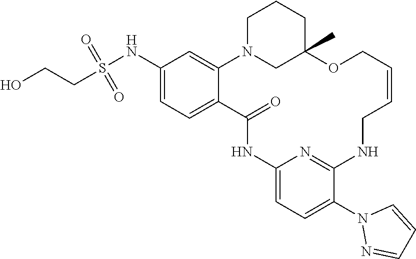 [SMILES: C[C@]12CCCN(C1)c1cc(NS(=O)(=O)CCO)ccc1C(=O)Nc1ccc(-n3cccn3)c(n1)NC/C=C\CO2] | 568.3 | 568.2323 |
| 117 | 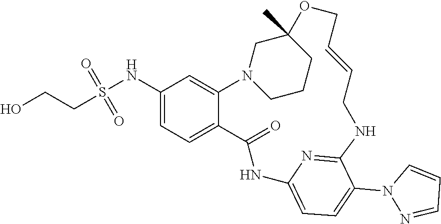 [SMILES: C[C@@]12CCCN(C1)c1cc(NS(=O)(=O)CCO)ccc1C(=O)Nc1ccc(-n3cccn3)c(n1)NC/C=C/CO2] | 568.3 | 568.2341 |
| 118 | 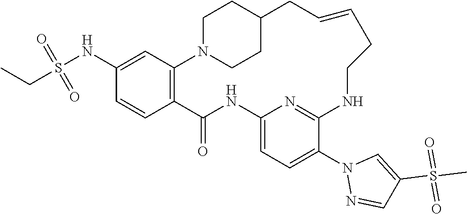 [SMILES: CCS(=O)(=O)Nc1ccc2c(c1)N1CCC(C/C=C/CCNc3nc(ccc3-n3cc(S(C)(=O)=O)cn3)NC2=O)CC1] | 614.2 | 614.2203 |
| 119 | 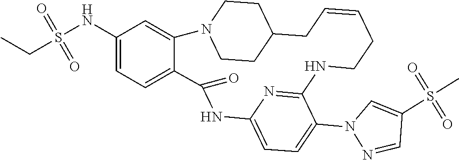 [SMILES: CCS(=O)(=O)Nc1ccc2c(c1)N1CCC(C/C=C\CCNc3nc(ccc3-n3cc(S(C)(=O)=O)cn3)NC2=O)CC1] | 614.2 | 614.2198 |
| 120 | 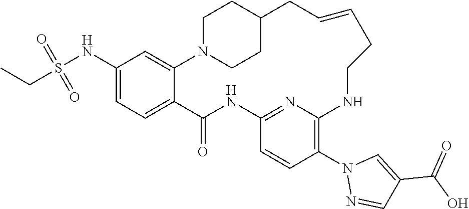 [SMILES: CCS(=O)(=O)Nc1ccc2c(c1)N1CCC(C/C=C/CCNc3nc(ccc3-n3cc(C(=O)O)cn3)NC2=O)CC1] | 580.2 | 580.2328 |
| 121 | 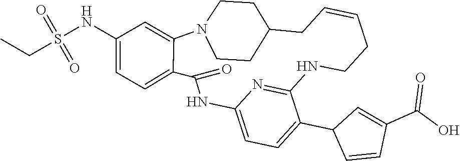 [SMILES: CCS(=O)(=O)Nc1ccc2c(c1)N1CCC(C/C=C\CCNc3nc(ccc3C3C=CC(C(=O)O)=C3)NC2=O)CC1] | 580.1 | |
| 122 | 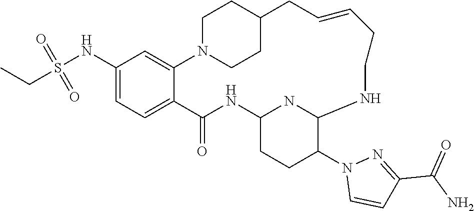 [SMILES: CCS(=O)(=O)Nc1ccc2c(c1)N1CCC(C/C=C/CCNC3NC(CCC3n3ccc(C(N)=O)n3)NC2=O)CC1] | 579.5 | 579.2487 |
| 123 | 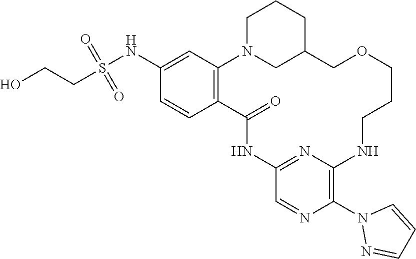 [SMILES: O=C1Nc2cnc(-n3cccn3)c(n2)NCCCOCC2CCCN(C2)c2cc(NS(=O)(=O)CCO)ccc21] | 556.1 | 556.2341 |
| 124 | 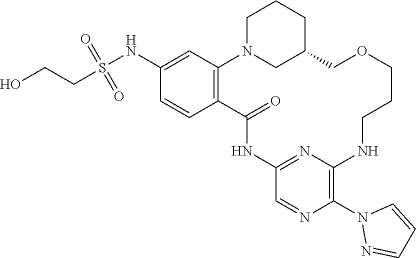 [SMILES: O=C1Nc2cnc(-n3cccn3)c(n2)NCCCOC[C@H]2CCCN(C2)c2cc(NS(=O)(=O)CCO)ccc21] | 556.3 | 556.2327 |
| 125 | 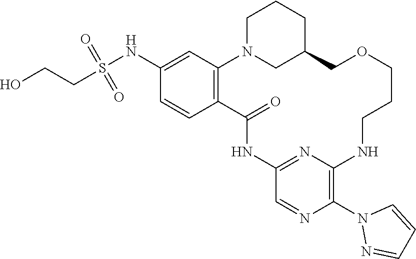 [SMILES: O=C1Nc2cnc(-n3cccn3)c(n2)NCCCOC[C@@H]2CCCN(C2)c2cc(NS(=O)(=O)CCO)ccc21] | 556.3 | 556.2339 |
| 126 | 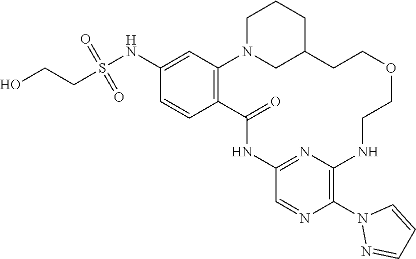 [SMILES: O=C1Nc2cnc(-n3cccn3)c(n2)NCCOCCC2CCCN(C2)c2cc(NS(=O)(=O)CCO)ccc21] | 556.2 | 556.2349 |
| 127 | 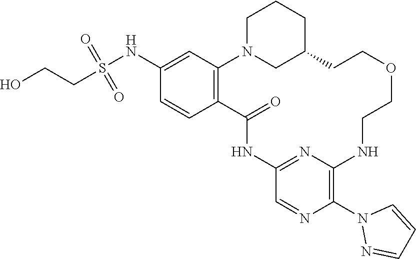 [SMILES: O=C1Nc2cnc(-n3cccn3)c(n2)NCCOCC[C@H]2CCCN(C2)c2cc(NS(=O)(=O)CCO)ccc21] | 556.2 | 556.2339 |
| 128 | 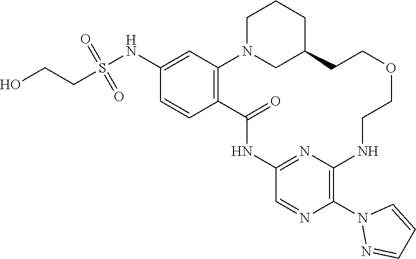 [SMILES: O=C1Nc2cnc(-n3cccn3)c(n2)NCCOCC[C@@H]2CCCN(C2)c2cc(NS(=O)(=O)CCO)ccc21] | 556.2 | 556.2334 |
| 129 | 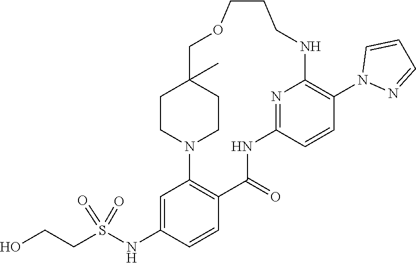 [SMILES: CC12CCN(CC1)c1cc(NS(=O)(=O)CCO)ccc1C(=O)Nc1ccc(-n3cccn3)c(n1)NCCCOC2] | 570.3 | 570.248 |
| 130 | 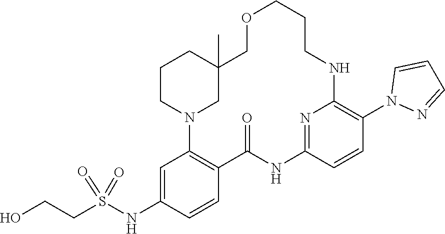 [SMILES: CC12CCCN(C1)c1cc(NS(=O)(=O)CCO)ccc1C(=O)Nc1ccc(-n3cccn3)c(n1)NCCCOC2] | 570.3 | 570.2484 |
| 131 | 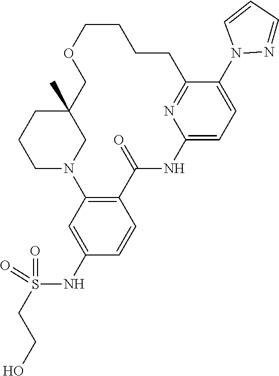 [SMILES: C[C@]12CCCN(C1)c1cc(NS(=O)(=O)CCO)ccc1C(=O)Nc1ccc(-n3cccn3)c(n1)CCCCOC2] | 570.3 | 570.2494 |
| 132 | 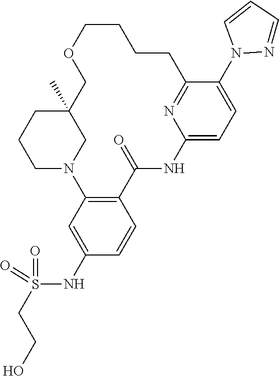 [SMILES: C[C@@]12CCCN(C1)c1cc(NS(=O)(=O)CCO)ccc1C(=O)Nc1ccc(-n3cccn3)c(n1)CCCCOC2] | 570.3 | 570.2486 |
| 134 | 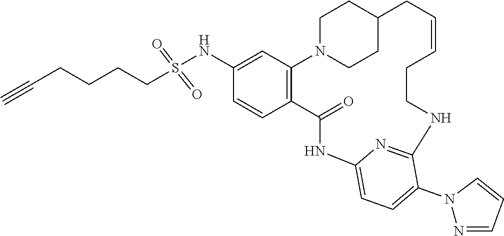 [SMILES: C#CCCCCS(=O)(=O)Nc1ccc2c(c1)N1CCC(C/C=C\CCNc3nc(ccc3-n3cccn3)NC2=O)CC1] | 588.7 | 588.2741 |
| 135 | 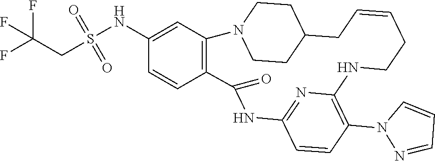 [SMILES: O=C1Nc2ccc(-n3cccn3)c(n2)NCC/C=C\CC2CCN(CC2)c2cc(NS(=O)(=O)CC(F)(F)F)ccc21] | 590.6 | |
| 136 | 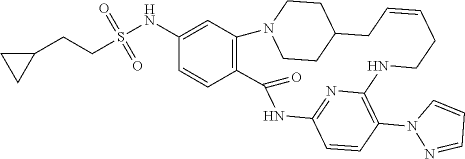 [SMILES: O=C1Nc2ccc(-n3cccn3)c(n2)NCC/C=C\CC2CCN(CC2)c2cc(NS(=O)(=O)CCC3CC3)ccc21] | 576.7 | 576.2726 |
| 137 | 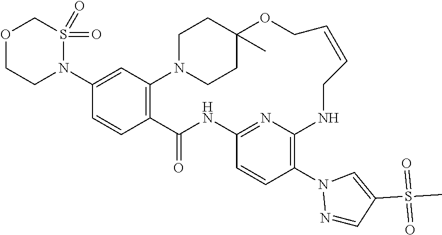 [SMILES: CC12CCN(CC1)c1cc(N3CCOCS3(=O)=O)ccc1C(=O)Nc1ccc(-n3cc(S(C)(=O)=O)cn3)c(n1)NC/C=C\CO2] | 658.5 | 658.2098 |
| 138 | 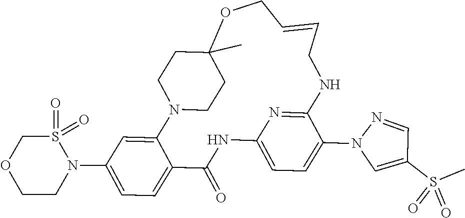 [SMILES: CC12CCN(CC1)c1cc(N3CCOCS3(=O)=O)ccc1C(=O)Nc1ccc(-n3cc(S(C)(=O)=O)cn3)c(n1)NC/C=C/CO2] | 658.5 | 658.2097 |
| 139 | 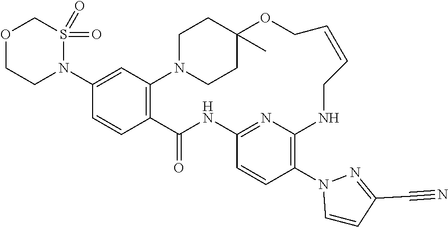 [SMILES: CC12CCN(CC1)c1cc(N3CCOCS3(=O)=O)ccc1C(=O)Nc1ccc(-n3ccc(C#N)n3)c(n1)NC/C=C\CO2] | 605.5 | 605.2277 |
| 140 | 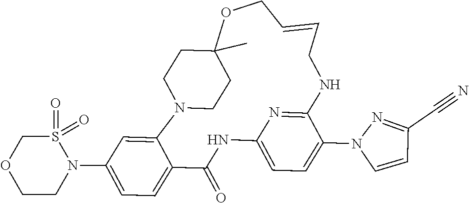 [SMILES: CC12CCN(CC1)c1cc(N3CCOCS3(=O)=O)ccc1C(=O)Nc1ccc(-n3ccc(C#N)n3)c(n1)NC/C=C/CO2] | 605.5 | 605.2273 |
| 141 | 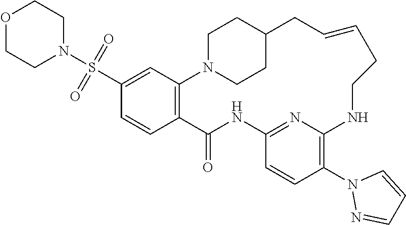 [SMILES: O=C1Nc2ccc(-n3cccn3)c(n2)NCC/C=C/CC2CCN(CC2)c2cc(S(=O)(=O)N3CCOCC3)ccc21] | 578.6 | 578.253 |
| 142 | 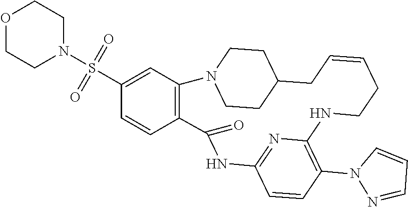 [SMILES: O=C1Nc2ccc(-n3cccn3)c(n2)NCC/C=C\CC2CCN(CC2)c2cc(S(=O)(=O)N3CCOCC3)ccc21] | 578.5 | |
| 143 | 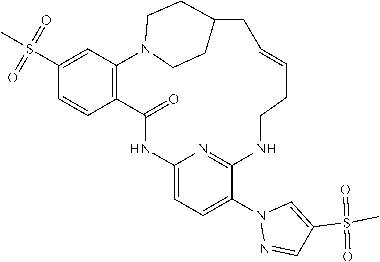 | 585.4 | 585.1926 |
| 144 | 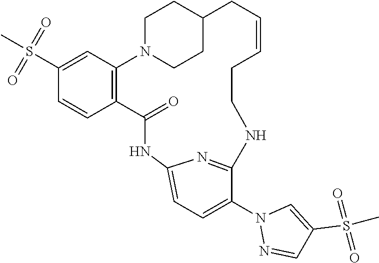 | 585.4 | 585.1932 |
| 145 | 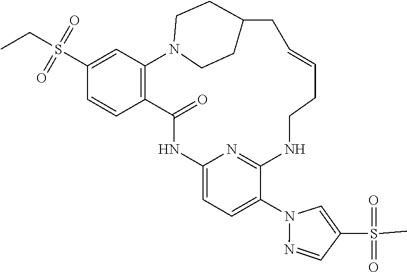 [SMILES: CCS(=O)(=O)c1ccc2c(c1)N1CCC(C/C=C/CCNc3nc(ccc3-n3cc(S(C)(=O)=O)cn3)NC2=O)CC1] | 599.5 | 599.2093 |
| 146 | 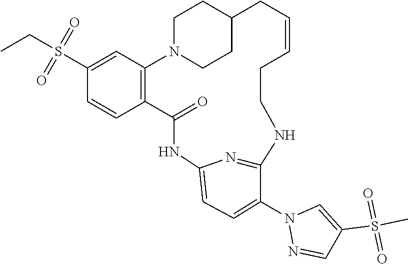 [SMILES: CCS(=O)(=O)c1ccc2c(c1)N1CCC(C/C=C\CCNc3nc(ccc3-n3cc(S(C)(=O)=O)cn3)NC2=O)CC1] | 599.0 | 599.2082 |
| 147 | 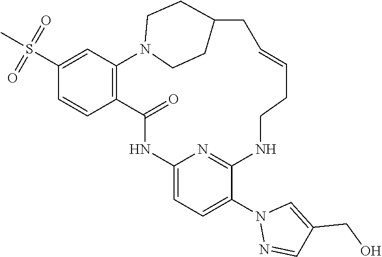 [SMILES: CS(=O)(=O)c1ccc2c(c1)N1CCC(C/C=C/CCNc3nc(ccc3-n3cc(CO)cn3)NC2=O)CC1] | 537.5 | 537.2262 |
| 148 | 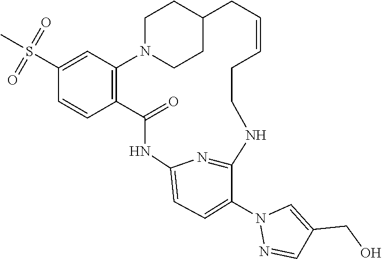 [SMILES: CS(=O)(=O)c1ccc2c(c1)N1CCC(C/C=C\CCNc3nc(ccc3-n3cc(CO)cn3)NC2=O)CC1] | 537.5 | 537.2251 |
| 149 | 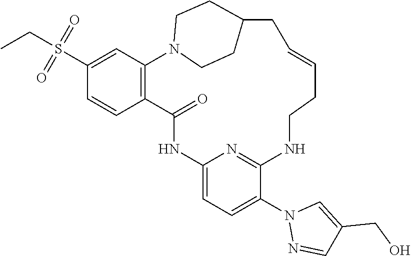 [SMILES: CCS(=O)(=O)c1ccc2c(c1)N1CCC(C/C=C/CCNc3nc(ccc3-n3cc(CO)cn3)NC2=O)CC1] | 551.3 | 551.2411 |
| 150 | 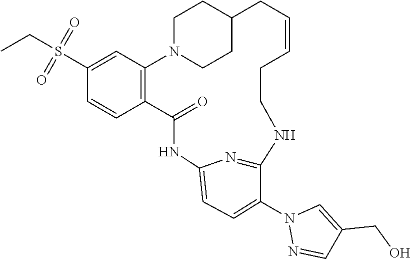 [SMILES: CCS(=O)(=O)c1ccc2c(c1)N1CCC(C/C=C\CCNc3nc(ccc3-n3cc(CO)cn3)NC2=O)CC1] | 551.1 | 551.2424 |
| 151 | 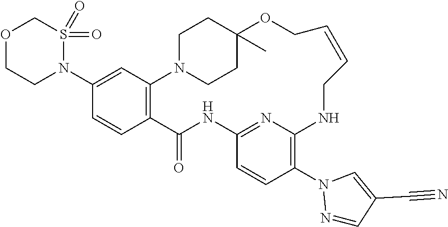 [SMILES: CC12CCN(CC1)c1cc(N3CCOCS3(=O)=O)ccc1C(=O)Nc1ccc(-n3cc(C#N)cn3)c(n1)NC/C=C\CO2] | 605.5 | 605.2268 |
| 152 | 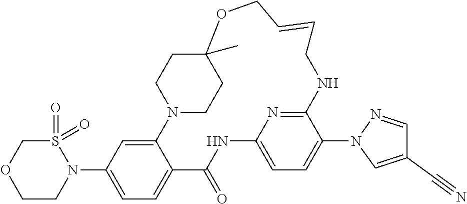 [SMILES: CC12CCN(CC1)c1cc(N3CCOCS3(=O)=O)ccc1C(=O)Nc1ccc(-n3cc(C#N)cn3)c(n1)NC/C=C/CO2] | 605.5 | 605.2269 |
| 153 | 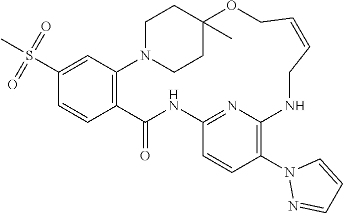 [SMILES: CC12CCN(CC1)c1cc(S(C)(=O)=O)ccc1C(=O)Nc1ccc(-n3cccn3)c(n1)NC/C=C\CO2] | 523.4 | 523.2113 |
| 154 | 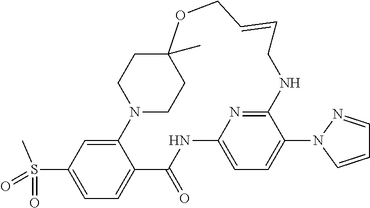 [SMILES: CC12CCN(CC1)c1cc(S(C)(=O)=O)ccc1C(=O)Nc1ccc(-n3cccn3)c(n1)NC/C=C/CO2] | 523.4 | 523.2107 |
| 155 | 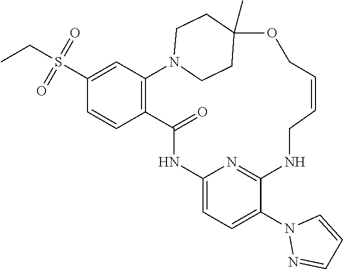 [SMILES: CCS(=O)(=O)c1ccc2c(c1)N1CCC(C)(CC1)OC/C=C\CNc1nc(ccc1-n1cccn1)NC2=O] | 537.1 | 537.2263 |
| 156 | 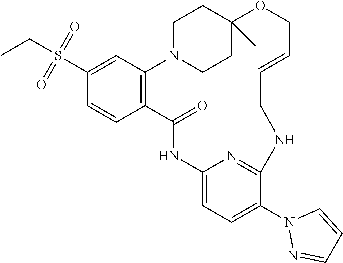 [SMILES: CCS(=O)(=O)c1ccc2c(c1)N1CCC(C)(CC1)OC/C=C/CNc1nc(ccc1-n1cccn1)NC2=O] | 537.1 | 537.2259 |
| 157 | 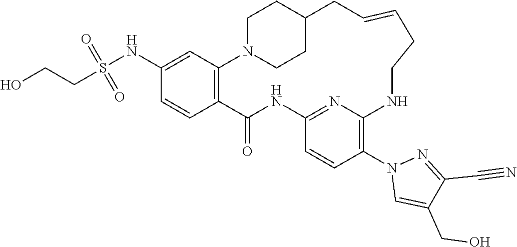 [SMILES: N#Cc1nn(-c2ccc3nc2NCC/C=C/CC2CCN(CC2)c2cc(NS(=O)(=O)CCO)ccc2C(=O)N3)cc1CO] | 607.6 | 607.2421 |
| 158 | 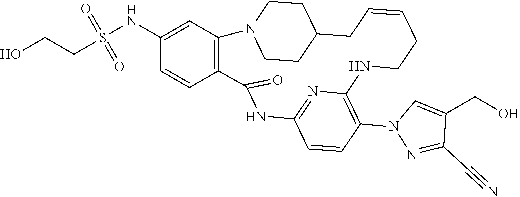 [SMILES: N#Cc1nn(-c2ccc3nc2NCC/C=C\CC2CCN(CC2)c2cc(NS(=O)(=O)CCO)ccc2C(=O)N3)cc1CO] | 607.5 | |
| 159 | 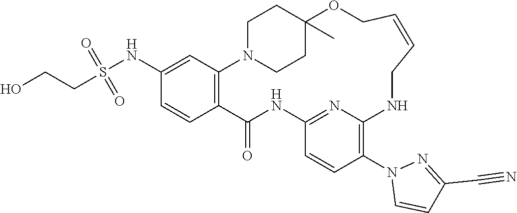 [SMILES: CC12CCN(CC1)c1cc(NS(=O)(=O)CCO)ccc1C(=O)Nc1ccc(-n3ccc(C#N)n3)c(n1)NC/C=C\CO2] | 593.5 | |
| 160 | 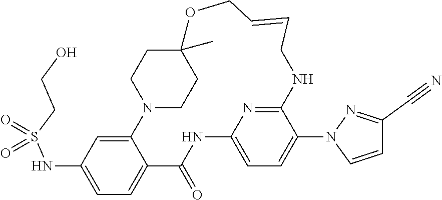 [SMILES: CC12CCN(CC1)c1cc(NS(=O)(=O)CCO)ccc1C(=O)Nc1ccc(-n3ccc(C#N)n3)c(n1)NC/C=C/CO2] | 593.6 | |
| 161 | 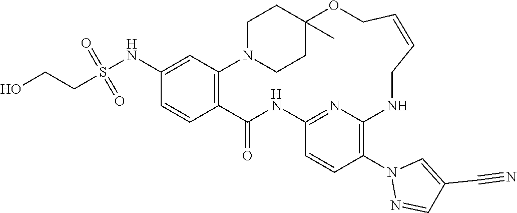 [SMILES: CC12CCN(CC1)c1cc(NS(=O)(=O)CCO)ccc1C(=O)Nc1ccc(-n3cc(C#N)cn3)c(n1)NC/C=C\CO2] | 593.5 | 593.227 |
| 162 | 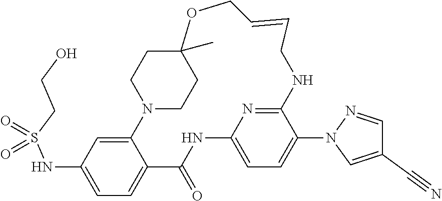 [SMILES: CC12CCN(CC1)c1cc(NS(=O)(=O)CCO)ccc1C(=O)Nc1ccc(-n3cc(C#N)cn3)c(n1)NC/C=C/CO2] | 593.5 | 593.2275 |
| 163 | 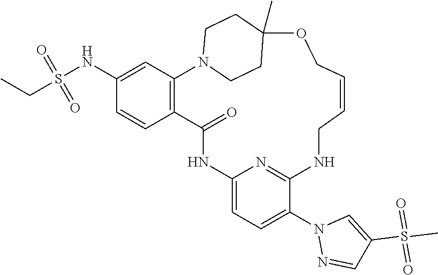 [SMILES: CCS(=O)(=O)Nc1ccc2c(c1)N1CCC(C)(CC1)OC/C=C\CNc1nc(ccc1-n1cc(S(C)(=O)=O)cn1)NC2=O] | 630.5 | 630.2128 |
| 164 | 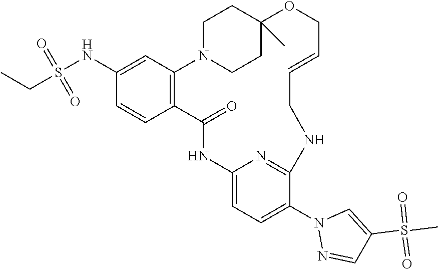 [SMILES: CCS(=O)(=O)Nc1ccc2c(c1)N1CCC(C)(CC1)OC/C=C/CNc1nc(ccc1-n1cc(S(C)(=O)=O)cn1)NC2=O] | 630.6 | 630.2137 |
| 165 | 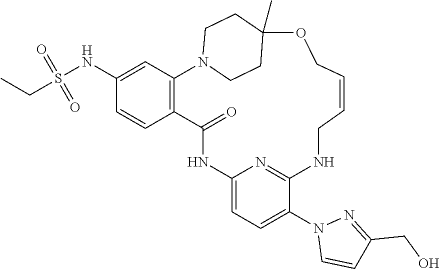 [SMILES: CCS(=O)(=O)Nc1ccc2c(c1)N1CCC(C)(CC1)OC/C=C\CNc1nc(ccc1-n1ccc(CO)n1)NC2=O] | 582.2 | 582.2475 |
| 166 | 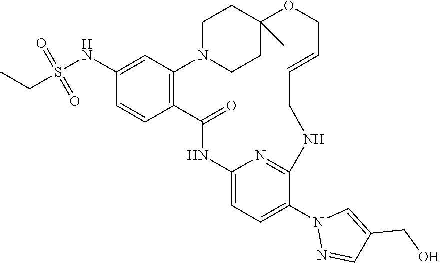 [SMILES: CCS(=O)(=O)Nc1ccc2c(c1)N1CCC(C)(CC1)OC/C=C/CNc1nc(ccc1-n1cc(CO)cn1)NC2=O] | 582.2 | 582.2481 |
| 167 | 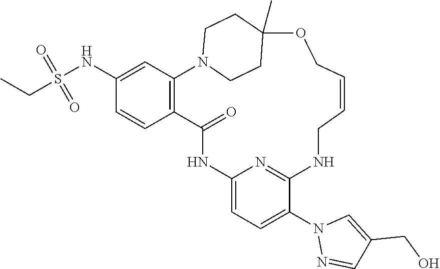 [SMILES: CCS(=O)(=O)Nc1ccc2c(c1)N1CCC(C)(CC1)OC/C=C\CNc1nc(ccc1-n1cc(CO)cn1)NC2=O] | 582.2 | 582.2474 |
| 168 | 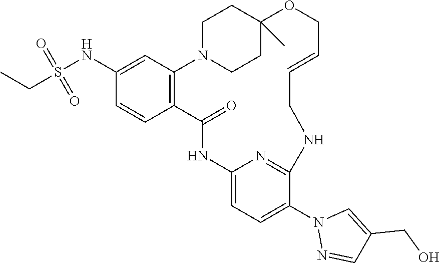 [SMILES: CCS(=O)(=O)Nc1ccc2c(c1)N1CCC(C)(CC1)OC/C=C/CNc1nc(ccc1-n1cc(CO)cn1)NC2=O] | 582.2 | 582.2477 |
| 169 | 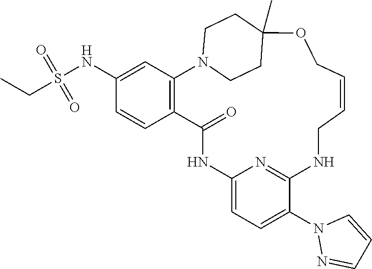 [SMILES: CCS(=O)(=O)Nc1ccc2c(c1)N1CCC(C)(CC1)OC/C=C\CNc1nc(ccc1-n1cccn1)NC2=O] | 552.3 | 552.2375 |
| 170 | 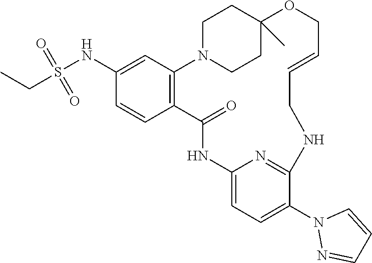 [SMILES: CCS(=O)(=O)Nc1ccc2c(c1)N1CCC(C)(CC1)OC/C=C/CNc1nc(ccc1-n1cccn1)NC2=O] | 552.3 | 552.237 |
| 171 | 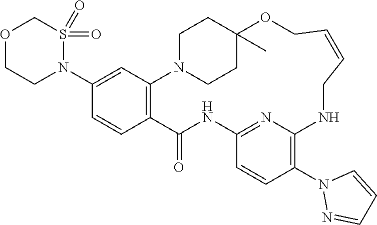 [SMILES: CC12CCN(CC1)c1cc(N3CCOCS3(=O)=O)ccc1C(=O)Nc1ccc(-n3cccn3)c(n1)NC/C=C\CO2] | 580.3 | 580.2323 |
| 172 | 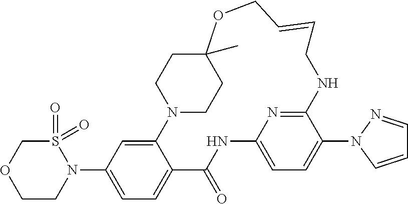 [SMILES: CC12CCN(CC1)c1cc(N3CCOCS3(=O)=O)ccc1C(=O)Nc1ccc(-n3cccn3)c(n1)NC/C=C/CO2] | 580.3 | 580.2329 |
| 173 | 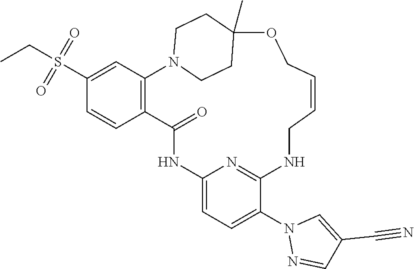 [SMILES: CCS(=O)(=O)c1ccc2c(c1)N1CCC(C)(CC1)OC/C=C\CNc1nc(ccc1-n1cc(C#N)cn1)NC2=O] | 562.2 | 562.2208 |
| 174 | 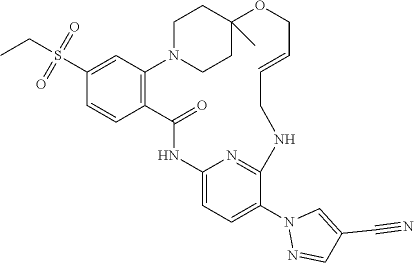 [SMILES: CCS(=O)(=O)c1ccc2c(c1)N1CCC(C)(CC1)OC/C=C/CNc1nc(ccc1-n1cc(C#N)cn1)NC2=O] | 562.2 | 562.2223 |
| 175 | 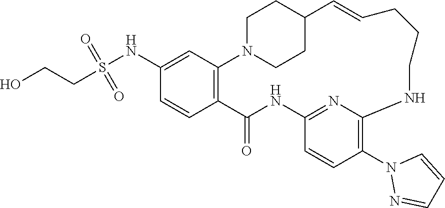 [SMILES: O=C1Nc2ccc(-n3cccn3)c(n2)NCCC/C=C/C2CCN(CC2)c2cc(NS(=O)(=O)CCO)ccc21] | 552.3 | 552.2387 |
| 176 | 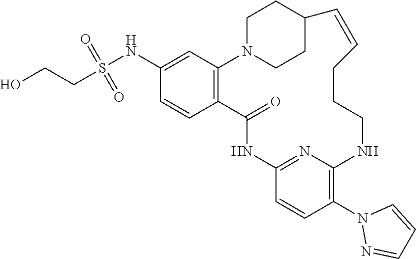 [SMILES: O=C1Nc2ccc(-n3cccn3)c(n2)NCCC/C=C\C2CCN(CC2)c2cc(NS(=O)(=O)CCO)ccc21] | 552.2 | 552.2384 |
| 177 | 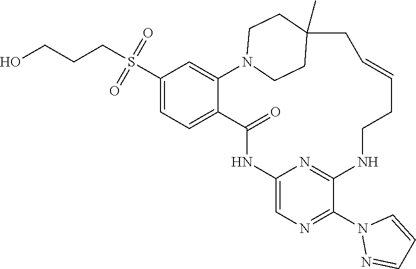 [SMILES: CC12C/C=C/CCNc3nc(cnc3-n3cccn3)NC(=O)c3ccc(S(=O)(=O)CCCO)cc3N(CC1)CC2] | 565.0 | 565.2581 |
| 178 | 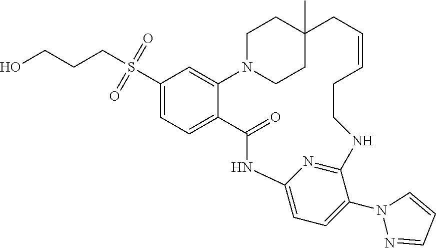 [SMILES: CC12C/C=C\CCNc3nc(ccc3-n3cccn3)NC(=O)c3ccc(S(=O)(=O)CCCO)cc3N(CC1)CC2] | 565.1 | 565.2586 |
| 179 | 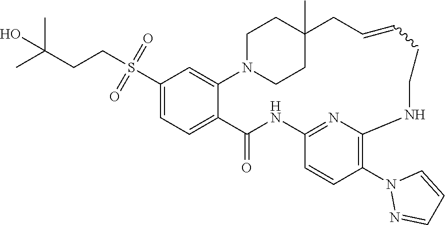 [SMILES: CC(C)(O)CCS(=O)(=O)c1ccc2c(c1)N1CCC(C)(CC=CCCNc3nc(ccc3-n3cccn3)NC2=O)CC1] | 593.2 | 593.2889 |
| 180 | 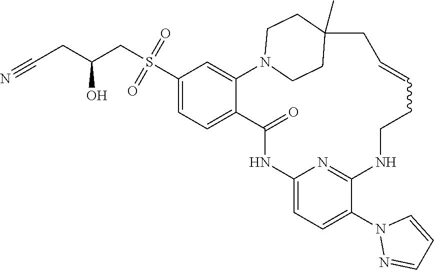 [SMILES: CC12CC=CCCNc3nc(ccc3-n3cccn3)NC(=O)c3ccc(S(=O)(=O)C[C@@H](O)CC#N)cc3N(CC1)CC2] | 590.1 | 590.2536 |
| 181 | 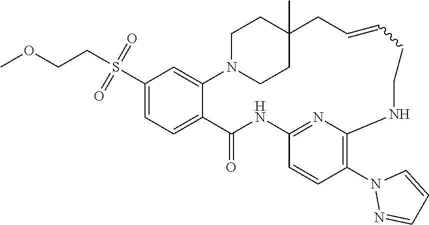 [SMILES: COCCS(=O)(=O)c1ccc2c(c1)N1CCC(C)(CC=CCCNc3nc(ccc3-n3cccn3)NC2=O)CC1] | 565.1 | 565.2582 |
| 182 | 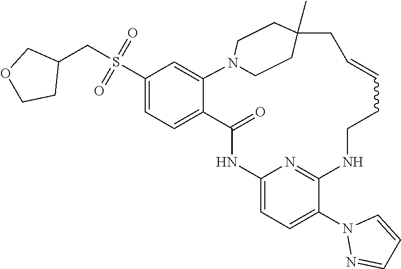 [SMILES: CC12CC=CCCNc3nc(ccc3-n3cccn3)NC(=O)c3ccc(S(=O)(=O)CC4CCOC4)cc3N(CC1)CC2] | 591.1 | 591.2736 |
| 183 | 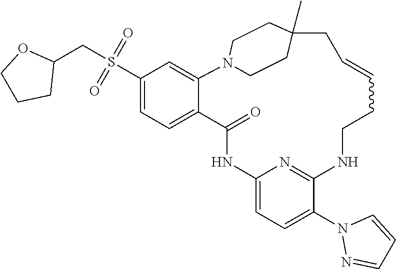 [SMILES: CC12CC=CCCNc3nc(ccc3-n3cccn3)NC(=O)c3ccc(S(=O)(=O)CC4CCCO4)cc3N(CC1)CC2] | 591.1 | 591.2728 |
| 184 | 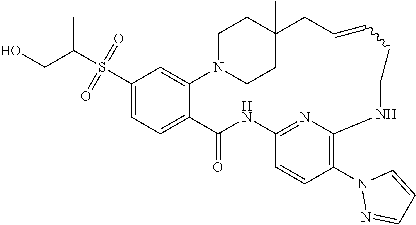 [SMILES: CC(CO)S(=O)(=O)c1ccc2c(c1)N1CCC(C)(CC=CCCNc3nc(ccc3-n3cccn3)NC2=O)CC1] | 565.1 | 565.2579 |
| 185 | 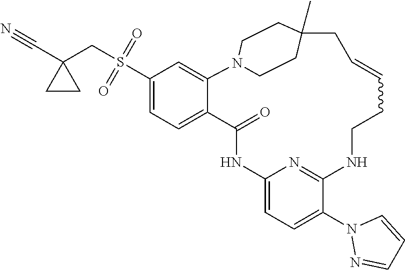 [SMILES: CC12CC=CCCNc3nc(ccc3-n3cccn3)NC(=O)c3ccc(S(=O)(=O)CC4(C#N)CC4)cc3N(CC1)CC2] | 586.1 | 586.2586 |
| 186 | 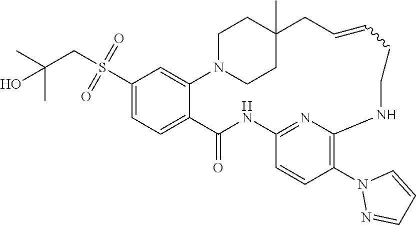 [SMILES: CC(C)(O)CS(=O)(=O)c1ccc2c(c1)N1CCC(C)(CC=CCCNc3nc(ccc3-n3cccn3)NC2=O)CC1] | 579.1 | 579.2732 |
| 187 | 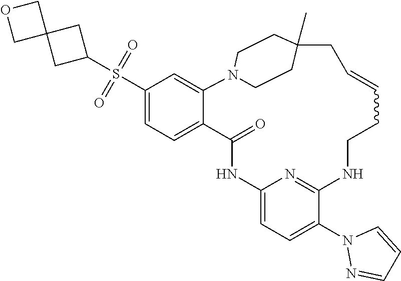 [SMILES: CC12CC=CCCNc3nc(ccc3-n3cccn3)NC(=O)c3ccc(S(=O)(=O)C4CC5(COC5)C4)cc3N(CC1)CC2] | 603.1 | 603.2734 |
| 188 | 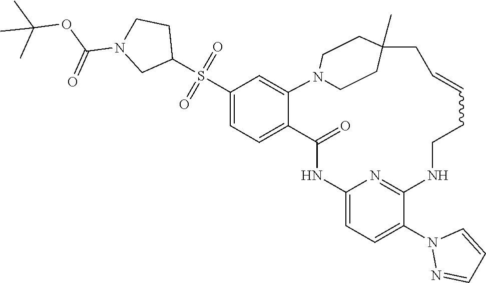 [SMILES: CC12CC=CCCNc3nc(ccc3-n3cccn3)NC(=O)c3ccc(S(=O)(=O)C4CCN(C(=O)OC(C)(C)C)C4)cc3N(CC1)CC2] | 676.1 | |
| 189 | 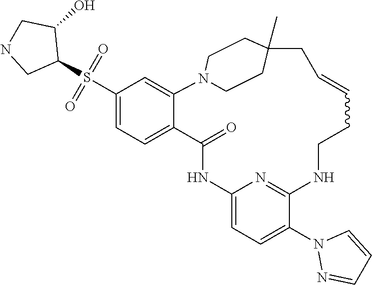 [SMILES: CC12CC=CCCNc3nc(ccc3-n3cccn3)NC(=O)c3ccc(S(=O)(=O)[C@H]4CNC[C@@H]4O)cc3N(CC1)CC2] | 593.1 | 593.2529 |
| 190 | 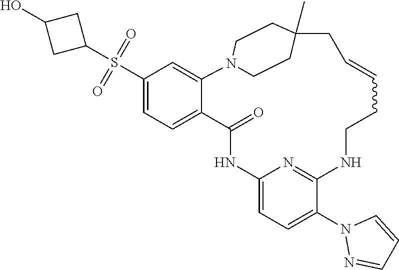 [SMILES: CC12CC=CCCNc3nc(ccc3-n3cccn3)NC(=O)c3ccc(S(=O)(=O)C4CC(O)C4)cc3N(CC1)CC2] | 577.1 | 577.2582 |
| 191 | 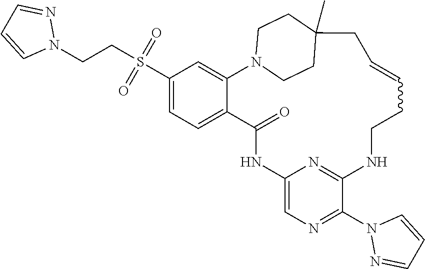 [SMILES: CC12CC=CCCNc3nc(cnc3-n3cccn3)NC(=O)c3ccc(S(=O)(=O)CCn4cccn4)cc3N(CC1)CC2] | 601.1 | 601.2693 |
| 192 | 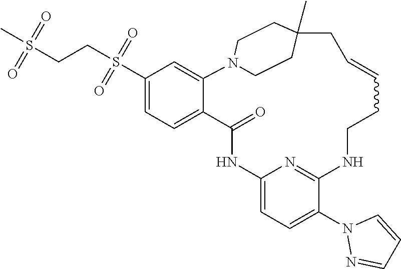 [SMILES: CC12CC=CCCNc3nc(ccc3-n3cccn3)NC(=O)c3ccc(S(=O)(=O)CCS(C)(=O)=O)cc3N(CC1)CC2] | 613.1 | 613.2256 |
| 193 | 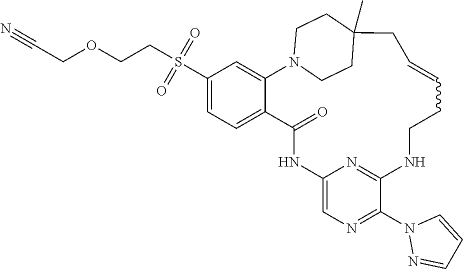 [SMILES: CC12CC=CCCNc3nc(cnc3-n3cccn3)NC(=O)c3ccc(S(=O)(=O)CCOCC#N)cc3N(CC1)CC2] | 590.1 | 590.2535 |
| 194 | 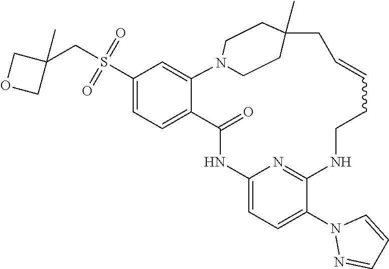 [SMILES: CC12CC=CCCNc3nc(ccc3-n3cccn3)NC(=O)c3ccc(S(=O)(=O)CC4(C)COC4)cc3N(CC1)CC2] | 591.2 | 591.2738 |
| 195 | 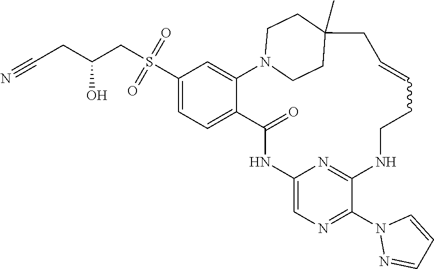 [SMILES: CC12CC=CCCNc3nc(cnc3-n3cccn3)NC(=O)c3ccc(S(=O)(=O)C[C@H](O)CC#N)cc3N(CC1)CC2] | 590.0 | 590.2532 |
| 196 | 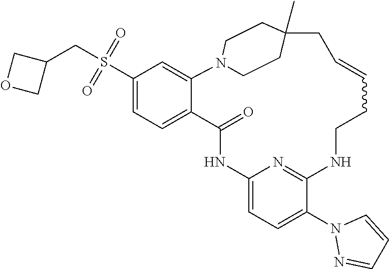 [SMILES: CC12CC=CCCNc3nc(ccc3-n3cccn3)NC(=O)c3ccc(S(=O)(=O)CC4COC4)cc3N(CC1)CC2] | 577.1 | 577.2586 |
| 197 | 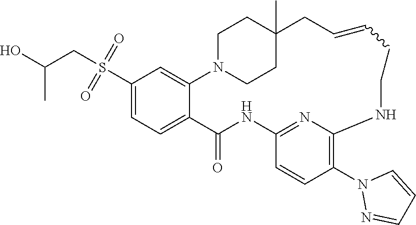 [SMILES: CC(O)CS(=O)(=O)c1ccc2c(c1)N1CCC(C)(CC=CCCNc3nc(ccc3-n3cccn3)NC2=O)CC1] | 565.1 | 565.258 |
| 198 | 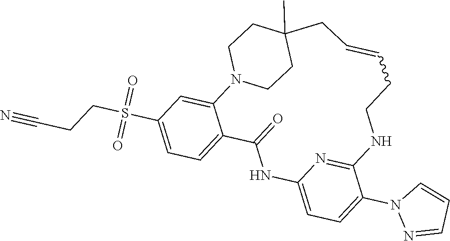 [SMILES: CC12CC=CCCNc3nc(ccc3-n3cccn3)NC(=O)c3ccc(S(=O)(=O)CCC#N)cc3N(CC1)CC2] | 560.1 | 560.2427 |
| 199 | 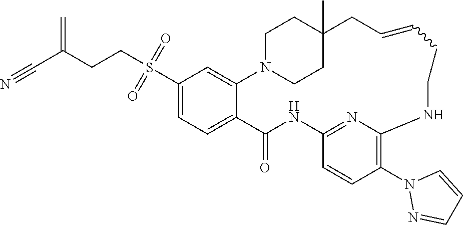 [SMILES: C=C(C#N)CCS(=O)(=O)c1ccc2c(c1)N1CCC(C)(CC=CCCNc3nc(ccc3-n3cccn3)NC2=O)CC1] | 586.1 | 586.2579 |
| 200 | 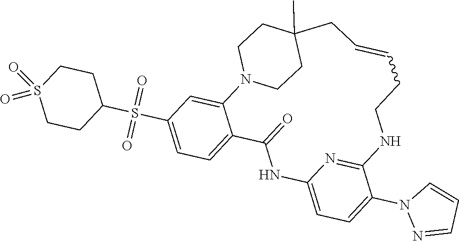 [SMILES: CC12CC=CCCNc3nc(ccc3-n3cccn3)NC(=O)c3ccc(S(=O)(=O)C4CCS(=O)(=O)CC4)cc3N(CC1)CC2] | 639.1 | 639.2397 |
| 201 | 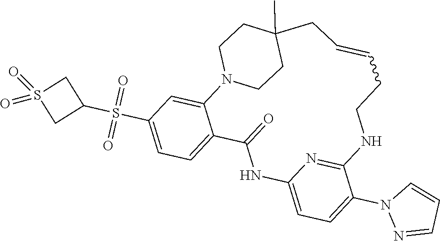 [SMILES: CC12CC=CCCNc3nc(ccc3-n3cccn3)NC(=O)c3ccc(S(=O)(=O)C4CS(=O)(=O)C4)cc3N(CC1)CC2] | 611.1 | 611.2089 |
| 202 | 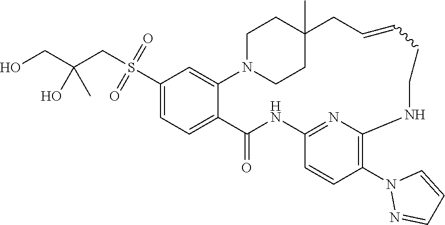 [SMILES: CC(O)(CO)CS(=O)(=O)c1ccc2c(c1)N1CCC(C)(CC=CCCNc3nc(ccc3-n3cccn3)NC2=O)CC1] | 595.1 | 595.2686 |
| 203 | 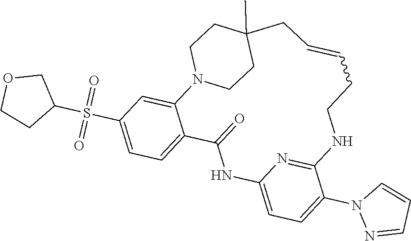 [SMILES: CC12CC=CCCNc3nc(ccc3-n3cccn3)NC(=O)c3ccc(S(=O)(=O)C4CCOC4)cc3N(CC1)CC2] | 577.1 | 577.2584 |
| 204 | 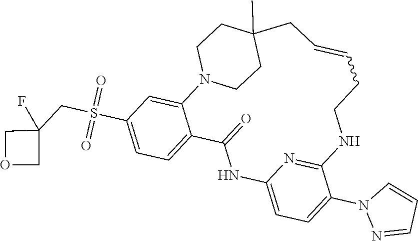 [SMILES: CC12CC=CCCNc3nc(ccc3-n3cccn3)NC(=O)c3ccc(S(=O)(=O)CC4(F)COC4)cc3N(CC1)CC2] | 595.1 | 595.2479 |
| 205 | 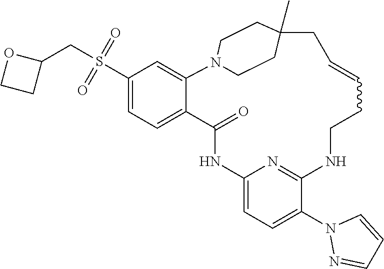 [SMILES: CC12CC=CCCNc3nc(ccc3-n3cccn3)NC(=O)c3ccc(S(=O)(=O)CC4CCO4)cc3N(CC1)CC2] | 577.1 | 577.2582 |
| 206 | 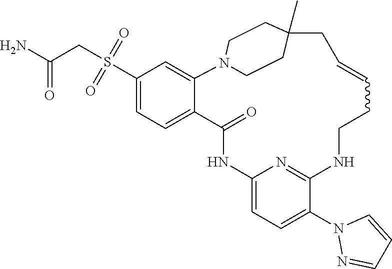 [SMILES: CC12CC=CCCNc3nc(ccc3-n3cccn3)NC(=O)c3ccc(S(=O)(=O)CC(N)=O)cc3N(CC1)CC2] | 564.1 | 564.2374 |
| 207 | 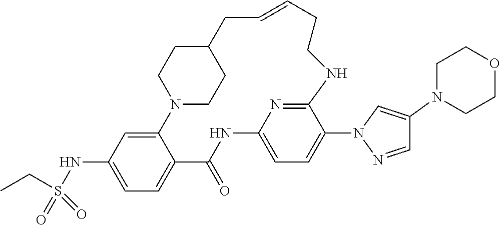 [SMILES: CCS(=O)(=O)Nc1ccc2c(c1)N1CCC(C/C=C/CCNc3nc(ccc3-n3cc(N4CCOCC4)cn3)NC2=O)CC1] | | 621.2956 |
| 208 | 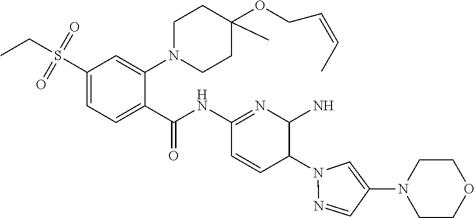 [SMILES: C/C=C\COC1(C)CCN(c2cc(S(=O)(=O)CC)ccc2C(=O)NC2=NC(N)C(n3cc(N4CCOCC4)cn3)C=C2)CC1] | 622.3 | 622.2796 |
| 209 | 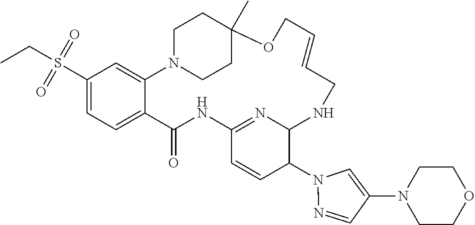 [SMILES: CCS(=O)(=O)c1ccc2c(c1)N1CCC(C)(CC1)OC/C=C/CNC1N=C(C=CC1n1cc(N3CCOCC3)cn1)NC2=O] | 622.3 | 622.2786 |
| 210 | 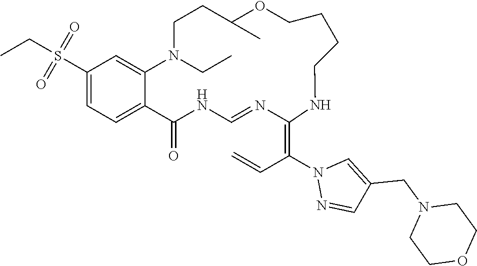 [SMILES: C=C/C(=C1\N=C\NC(=O)c2ccc(S(=O)(=O)CC)cc2N(CC)CCC(C)OCCCCN1)n1cc(CN2CCOCC2)cn1] | 636.4 | 636.2955 |
| 211 | 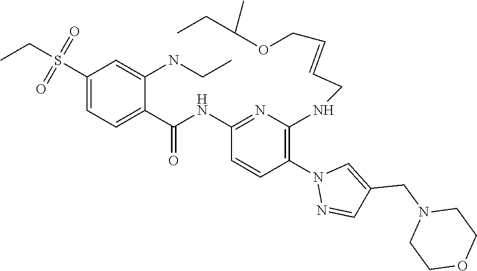 [SMILES: CCNc1cc(S(=O)(=O)CC)ccc1C(=O)Nc1ccc(-n2cc(CN3CCOCC3)cn2)c(NC/C=C/COC(C)CC)n1] | 636.3 | |
| 212 | 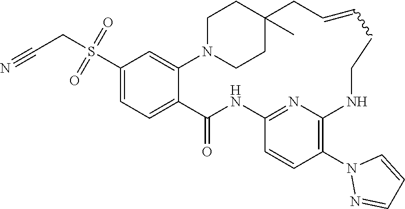 [SMILES: CC12CC=CCCNc3nc(ccc3-n3cccn3)NC(=O)c3ccc(S(=O)(=O)CC#N)cc3N(CC1)CC2] | 560 | |
| 213 | 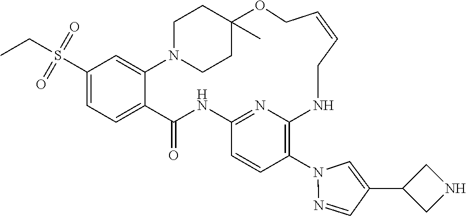 [SMILES: CCS(=O)(=O)c1ccc2c(c1)N1CCC(C)(CC1)OC/C=C\CNc1nc(ccc1-n1cc(C3CNC3)cn1)NC2=O] | 592.3 | 592.2675 |
| 214 | 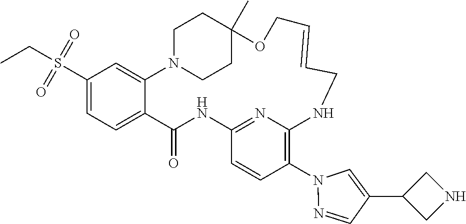 [SMILES: CCS(=O)(=O)c1ccc2c(c1)N1CCC(C)(CC1)OC/C=C/CNc1nc(ccc1-n1cc(C3CNC3)cn1)NC2=O] | 592.3 | 592.2675 |
| 215 | 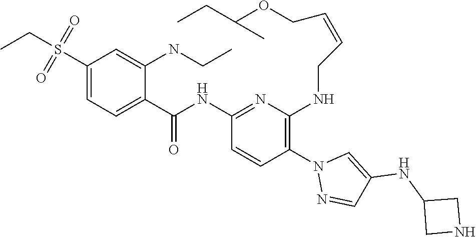 [SMILES: CCNc1cc(S(=O)(=O)CC)ccc1C(=O)Nc1ccc(-n2cc(NC3CNC3)cn2)c(NC/C=C\COC(C)CC)n1] | 607.4 | 607.2787 |
| 216 | 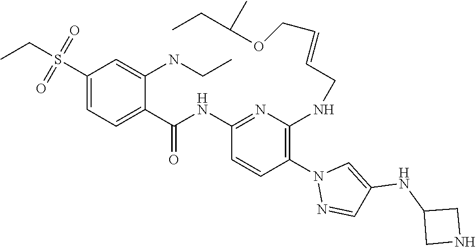 [SMILES: CCNc1cc(S(=O)(=O)CC)ccc1C(=O)Nc1ccc(-n2cc(NC3CNC3)cn2)c(NC/C=C/COC(C)CC)n1] | 607.4 | 607.2783 |
| 217 | 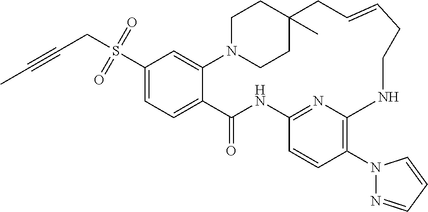 [SMILES: CC#CCS(=O)(=O)c1ccc2c(c1)N1CCC(C)(C/C=C/CCNc3nc(ccc3-n3cccn3)NC2=O)CC1] | 559.0 | 559.2461 |
| 218 | 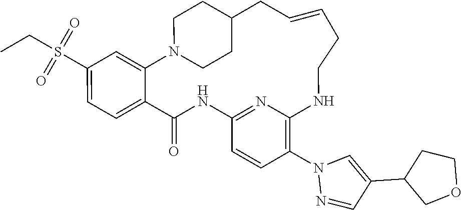 [SMILES: CCS(=O)(=O)c1ccc2c(c1)N1CCC(C/C=C/CCNc3nc(ccc3-n3cc(C4CCOC4)cn3)NC2=O)CC1] | 591.1 | 591.2739 |
| 219 | 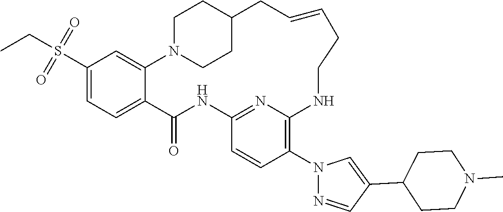 [SMILES: CCS(=O)(=O)c1ccc2c(c1)N1CCC(C/C=C/CCNc3nc(ccc3-n3cc(C4CCN(C)CC4)cn3)NC2=O)CC1] | 618.2 | 618.3216 |
| 220 | 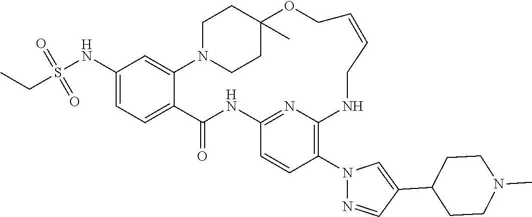 [SMILES: CCS(=O)(=O)Nc1ccc2c(c1)N1CCC(C)(CC1)OC/C=C\CNc1nc(ccc1-n1cc(C3CCN(C)CC3)cn1)NC2=O] | 649.2 | 649.327 |
| 221 | 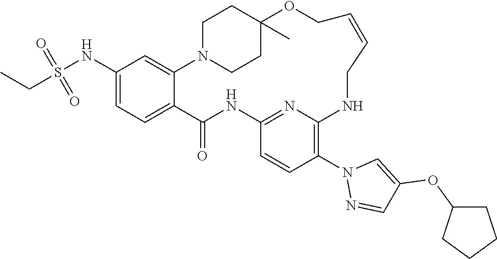 [SMILES: CCS(=O)(=O)Nc1ccc2c(c1)N1CCC(C)(CC1)OC/C=C\CNc1nc(ccc1-n1cc(OC3CCCC3)cn1)NC2=O] | 636.2 | 636.2952 |
| 222 | 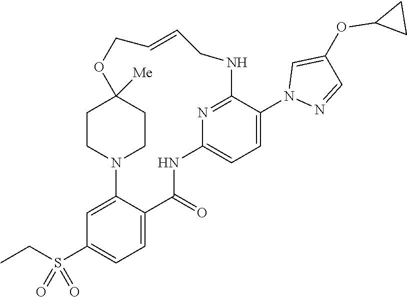 [SMILES: CCS(=O)(=O)c1ccc2c(c1)N1CCC(C)(CC1)OC/C=C/CNc1nc(ccc1-n1cc(OC3CC3)cn1)NC2=O] | 593.3 | |
| 223 | 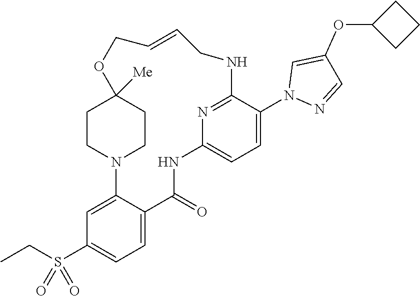 [SMILES: CCS(=O)(=O)c1ccc2c(c1)N1CCC(C)(CC1)OC/C=C/CNc1nc(ccc1-n1cc(OC3CCC3)cn1)NC2=O] | 607.2 | 607.2686 |
| 224 | 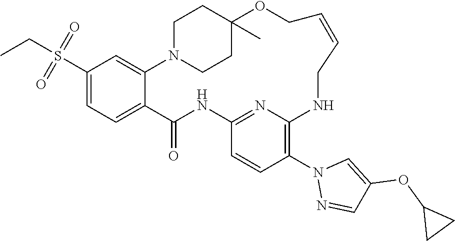 [SMILES: CCS(=O)(=O)c1ccc2c(c1)N1CCC(C)(CC1)OC/C=C\CNc1nc(ccc1-n1cc(OC3CC3)cn1)NC2=O] | 593.4 | |
| 225 | 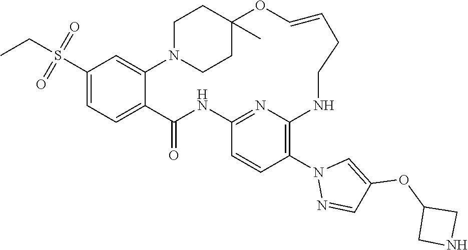 [SMILES: CCS(=O)(=O)c1ccc2c(c1)N1CCC(C)(CC1)O/C=C/CCNc1nc(ccc1-n1cc(OC3CNC3)cn1)NC2=O] | 608.3 | 608.2649 |
| 226 | 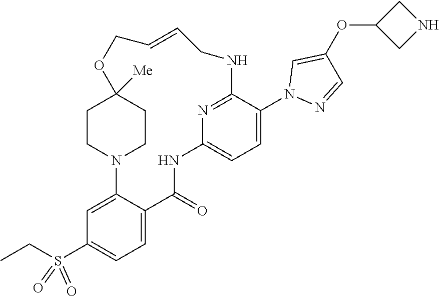 [SMILES: CCS(=O)(=O)c1ccc2c(c1)N1CCC(C)(CC1)OC/C=C/CNc1nc(ccc1-n1cc(OC3CNC3)cn1)NC2=O] | 608.3 | 608.2646 |
| 227 | 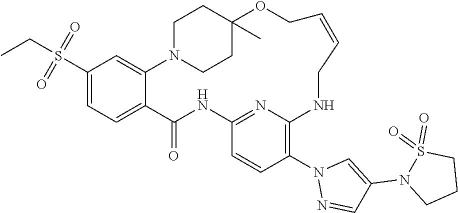 [SMILES: CCS(=O)(=O)c1ccc2c(c1)N1CCC(C)(CC1)OC/C=C\CNc1nc(ccc1-n1cc(N3CCCS3(=O)=O)cn1)NC2=O] | 656.3 | 656.2302 |
| 228 | 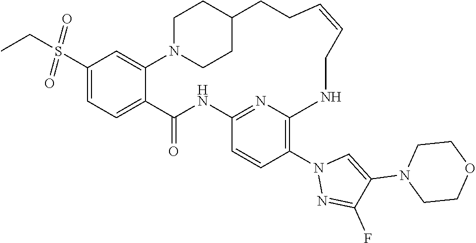 [SMILES: CCS(=O)(=O)c1ccc2c(c1)N1CCC(CC/C=C\CNc3nc(ccc3-n3cc(N4CCOCC4)c(F)n3)NC2=O)CC1] | 640.3 | 640.2695 |
| 229 | 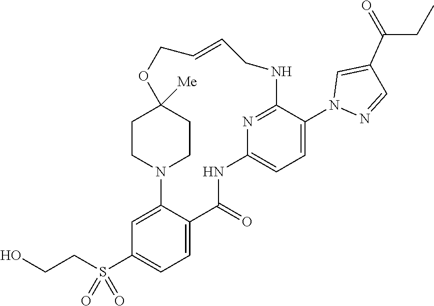 [SMILES: CCC(=O)c1cnn(-c2ccc3nc2NC/C=C/COC2(C)CCN(CC2)c2cc(S(=O)(=O)CCO)ccc2C(=O)N3)c1] | 609.3 | 609.2477 |
| 230 | 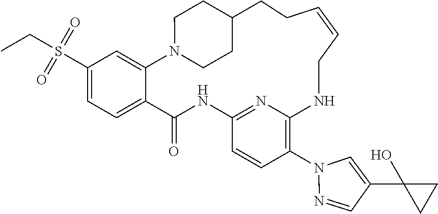 [SMILES: CCS(=O)(=O)c1ccc2c(c1)N1CCC(CC/C=C\CNc3nc(ccc3-n3cc(C4(O)CC4)cn3)NC2=O)CC1] | 593.3 | 593.2526 |
| 231 | 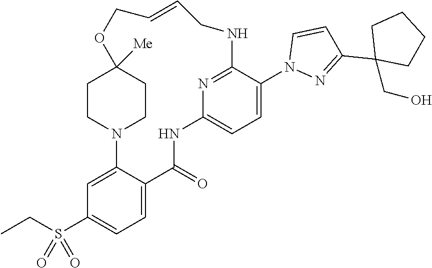 [SMILES: CCS(=O)(=O)c1ccc2c(c1)N1CCC(C)(CC1)OC/C=C/CNc1nc(ccc1-n1ccc(C3(CO)CCCC3)n1)NC2=O] | 635.2 | 635.2995 |
| 232 | 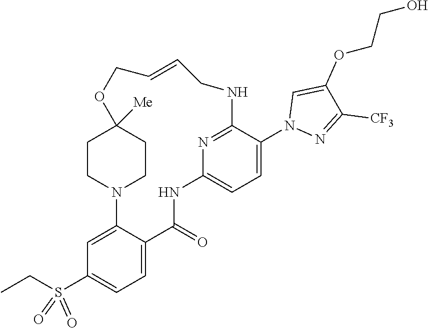 [SMILES: CCS(=O)(=O)c1ccc2c(c1)N1CCC(C)(CC1)OC/C=C/CNc1nc(ccc1-n1cc(OCCO)c(C(F)(F)F)n1)NC2=O] | 665.2 | 665.2351 |
| 233 | 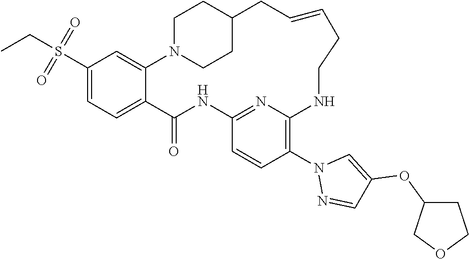 [SMILES: CCS(=O)(=O)c1ccc2c(c1)N1CCC(C/C=C/CCNc3nc(ccc3-n3cc(OC4CCOC4)cn3)NC2=O)CC1] | 607.2 | 607.2692 |
| 234 | 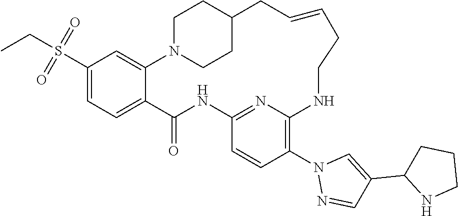 [SMILES: CCS(=O)(=O)c1ccc2c(c1)N1CCC(C/C=C/CCNc3nc(ccc3-n3cc(C4CCCN4)cn3)NC2=O)CC1] | 590.5 | 590.2895 |
| 235 | 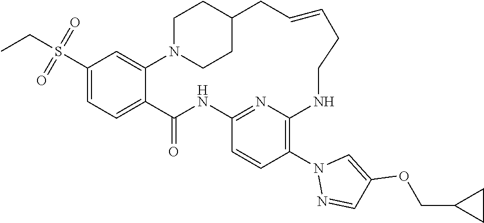 [SMILES: CCS(=O)(=O)c1ccc2c(c1)N1CCC(C/C=C/CCNc3nc(ccc3-n3cc(OCC4CC4)cn3)NC2=O)CC1] | 591.2 | 591.2731 |
| 236 | 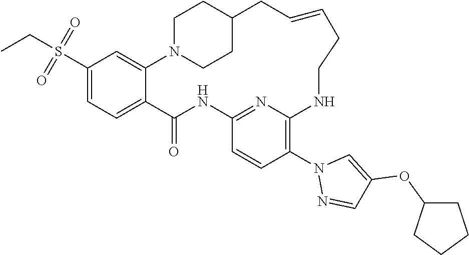 [SMILES: CCS(=O)(=O)c1ccc2c(c1)N1CCC(C/C=C/CCNc3nc(ccc3-n3cc(OC4CCCC4)cn3)NC2=O)CC1] | 605.2 | 605.2887 |
| 237 | 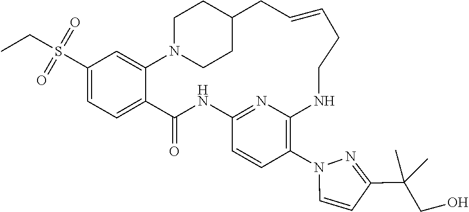 [SMILES: CCS(=O)(=O)c1ccc2c(c1)N1CCC(C/C=C/CCNc3nc(ccc3-n3ccc(C(C)(C)CO)n3)NC2=O)CC1] | 593.3 | 593.2888 |
| 238 | 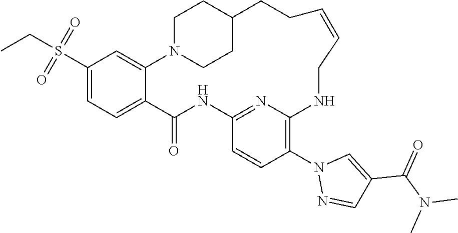 [SMILES: CCS(=O)(=O)c1ccc2c(c1)N1CCC(CC/C=C\CNc3nc(ccc3-n3cc(C(=O)N(C)C)cn3)NC2=O)CC1] | 592.2 | 592.2686 |
| 239 | 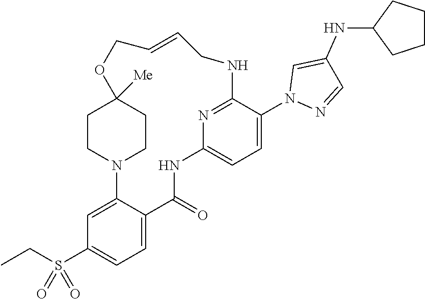 [SMILES: CCS(=O)(=O)c1ccc2c(c1)N1CCC(C)(CC1)OC/C=C/CNc1nc(ccc1-n1cc(NC3CCCC3)cn1)NC2=O] | 620.2 | 620.2998 |
| 240 | 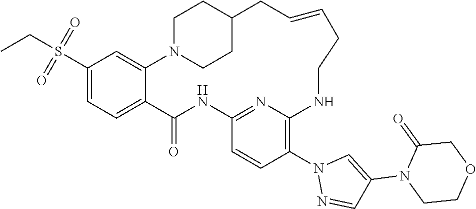 [SMILES: CCS(=O)(=O)c1ccc2c(c1)N1CCC(C/C=C/CCNc3nc(ccc3-n3cc(N4CCOCC4=O)cn3)NC2=O)CC1] | 620.2 | 620.2642 |
| 241 | 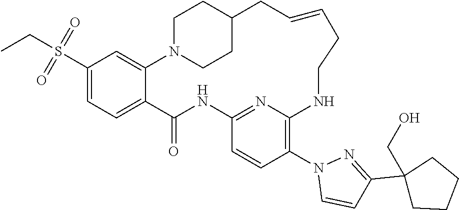 [SMILES: CCS(=O)(=O)c1ccc2c(c1)N1CCC(C/C=C/CCNc3nc(ccc3-n3ccc(C4(CO)CCCC4)n3)NC2=O)CC1] | 619.2 | 619.3052 |
| 242 | 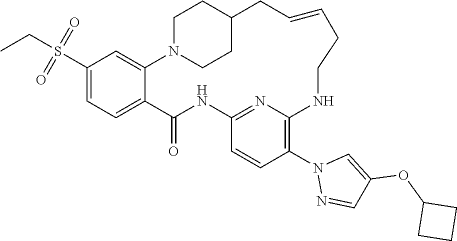 [SMILES: CCS(=O)(=O)c1ccc2c(c1)N1CCC(C/C=C/CCNc3nc(ccc3-n3cc(OC4CCC4)cn3)NC2=O)CC1] | 591.2 | 591.2738 |
| 243 | 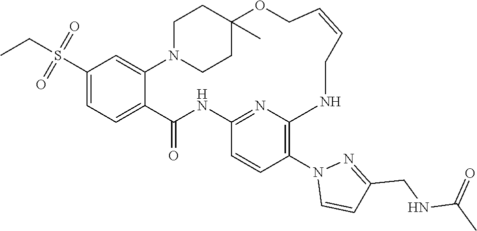 [SMILES: CCS(=O)(=O)c1ccc2c(c1)N1CCC(C)(CC1)OC/C=C\CNc1nc(ccc1-n1ccc(CNC(C)=O)n1)NC2=O] | 608.2 | 608.2642 |
| 244 | 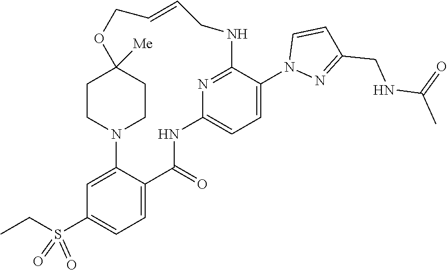 [SMILES: CCS(=O)(=O)c1ccc2c(c1)N1CCC(C)(CC1)OC/C=C/CNc1nc(ccc1-n1ccc(CNC(C)=O)n1)NC2=O] | 608.2 | 608.2644 |
| 245 | 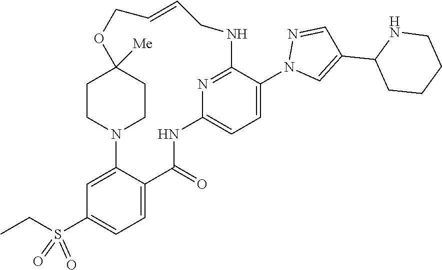 [SMILES: CCS(=O)(=O)c1ccc2c(c1)N1CCC(C)(CC1)OC/C=C/CNc1nc(ccc1-n1cc(C3CCCCN3)cn1)NC2=O] | 620.2 | 620.3001 |
| 246 | 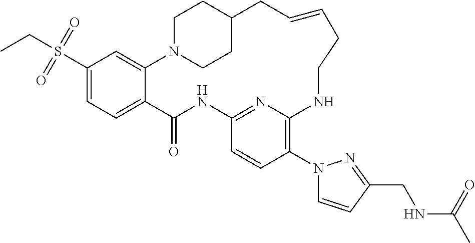 [SMILES: CCS(=O)(=O)c1ccc2c(c1)N1CCC(C/C=C/CCNc3nc(ccc3-n3ccc(CNC(C)=O)n3)NC2=O)CC1] | 592.1 | 592.2687 |
| 247 | 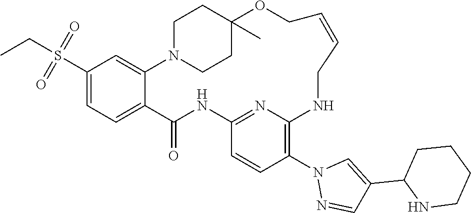 [SMILES: CCS(=O)(=O)c1ccc2c(c1)N1CCC(C)(CC1)OC/C=C\CNc1nc(ccc1-n1cc(C3CCCCN3)cn1)NC2=O] | 620.2 | 620.3014 |
| 248 | 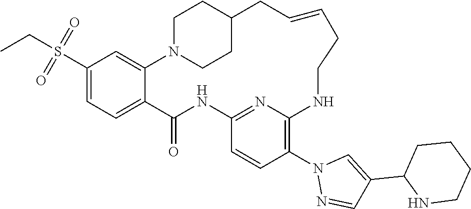 [SMILES: CCS(=O)(=O)c1ccc2c(c1)N1CCC(C/C=C/CCNc3nc(ccc3-n3cc(C4CCCCN4)cn3)NC2=O)CC1] | 604.2 | 604.3065 |
| 249 | 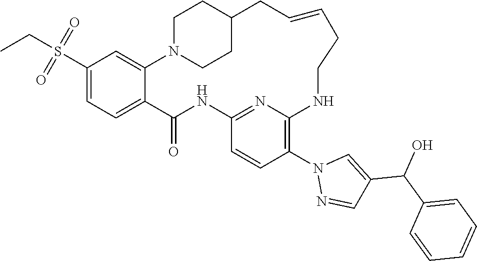 [SMILES: CCS(=O)(=O)c1ccc2c(c1)N1CCC(C/C=C/CCNc3nc(ccc3-n3cc(C(O)c4ccccc4)cn3)NC2=O)CC1] | 627.2 | 627.2735 |
| 250 | 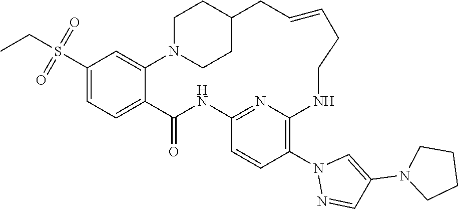 [SMILES: CCS(=O)(=O)c1ccc2c(c1)N1CCC(C/C=C/CCNc3nc(ccc3-n3cc(N4CCCC4)cn3)NC2=O)CC1] | 590.2 | 590.2892 |
| 251 | 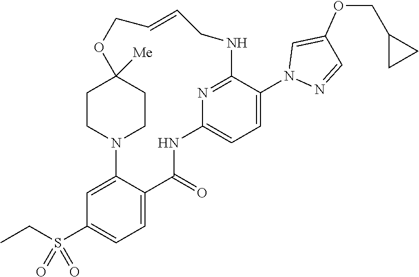 [SMILES: CCS(=O)(=O)c1ccc2c(c1)N1CCC(C)(CC1)OC/C=C/CNc1nc(ccc1-n1cc(OCC3CC3)cn1)NC2=O] | 607.2 | 607.2684 |
| 252 | 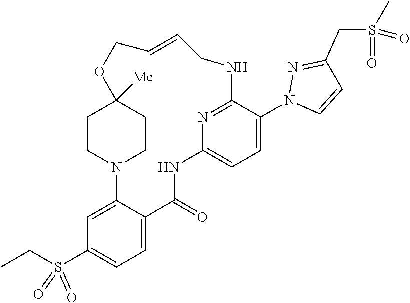 [SMILES: CCS(=O)(=O)c1ccc2c(c1)N1CCC(C)(CC1)OC/C=C/CNc1nc(ccc1-n1ccc(CS(C)(=O)=O)n1)NC2=O] | 629.1 | 629.2193 |
| 253 | 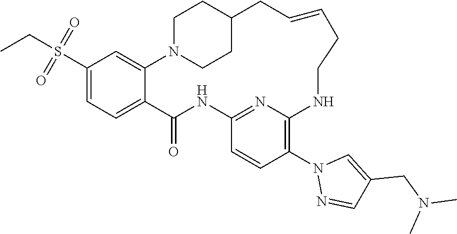 [SMILES: CCS(=O)(=O)c1ccc2c(c1)N1CCC(C/C=C/CCNc3nc(ccc3-n3cc(CN(C)C)cn3)NC2=O)CC1] | 578.2 | 578.2901 |
| 254 | 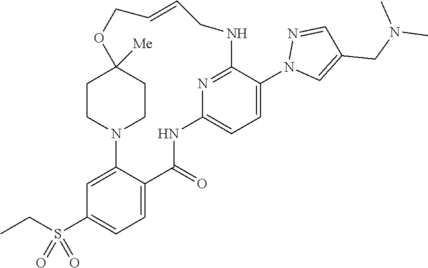 [SMILES: CCS(=O)(=O)c1ccc2c(c1)N1CCC(C)(CC1)OC/C=C/CNc1nc(ccc1-n1cc(CN(C)C)cn1)NC2=O] | 594.2 | 594.2848 |
| 255 | 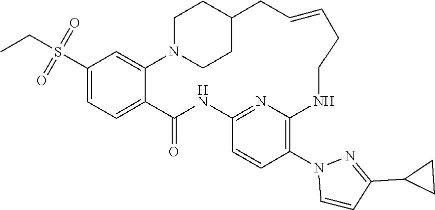 [SMILES: CCS(=O)(=O)c1ccc2c(c1)N1CCC(C/C=C/CCNc3nc(ccc3-n3ccc(C4CC4)n3)NC2=O)CC1] | 561.2 | 561.2635 |
| 256 | 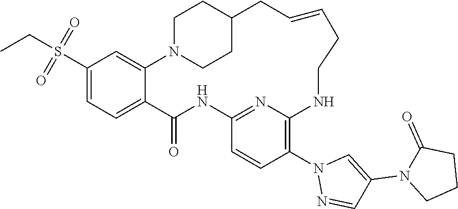 [SMILES: CCS(=O)(=O)c1ccc2c(c1)N1CCC(C/C=C/CCNc3nc(ccc3-n3cc(N4CCCC4=O)cn3)NC2=O)CC1] | 604.2 | 604.269 |
| 257 | 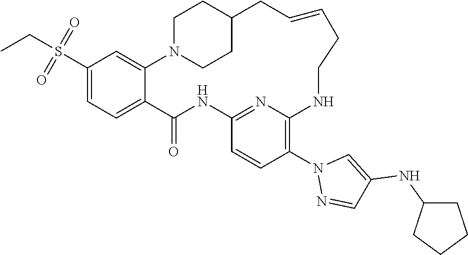 [SMILES: CCS(=O)(=O)c1ccc2c(c1)N1CCC(C/C=C/CCNc3nc(ccc3-n3cc(NC4CCCC4)cn3)NC2=O)CC1] | 604.2 | 604.3052 |
| 258 | 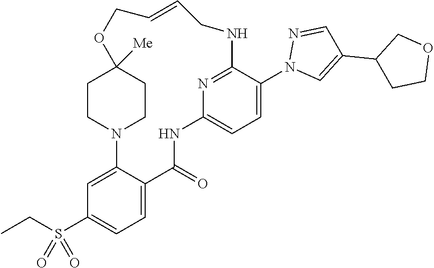 [SMILES: CCS(=O)(=O)c1ccc2c(c1)N1CCC(C)(CC1)OC/C=C/CNc1nc(ccc1-n1cc(C3CCOC3)cn1)NC2=O] | 607.2 | 607.2689 |
| 259 | 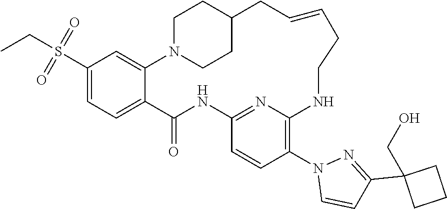 [SMILES: CCS(=O)(=O)c1ccc2c(c1)N1CCC(C/C=C/CCNc3nc(ccc3-n3ccc(C4(CO)CCC4)n3)NC2=O)CC1] | 605.2 | 605.2901 |
| 260 | 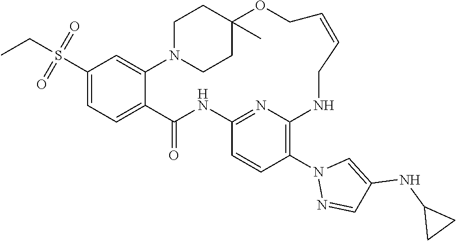 [SMILES: CCS(=O)(=O)c1ccc2c(c1)N1CCC(C)(CC1)OC/C=C\CNc1nc(ccc1-n1cc(NC3CC3)cn1)NC2=O] | 592.3 | 592.2669 |
| 261 | 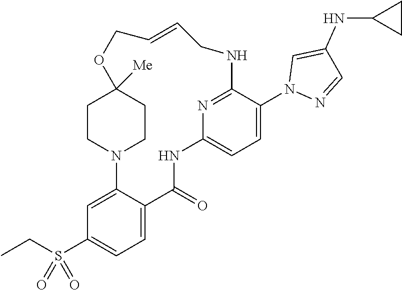 [SMILES: CCS(=O)(=O)c1ccc2c(c1)N1CCC(C)(CC1)OC/C=C/CNc1nc(ccc1-n1cc(NC3CC3)cn1)NC2=O] | 592.3 | 592.2682 |
| 262 | 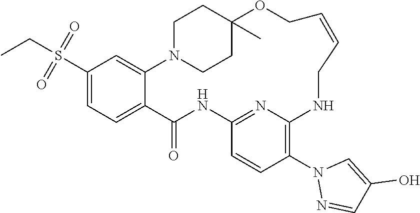 [SMILES: CCS(=O)(=O)c1ccc2c(c1)N1CCC(C)(CC1)OC/C=C\CNc1nc(ccc1-n1cc(O)cn1)NC2=O] | 553.3 | 553.2223 |
| 263 | 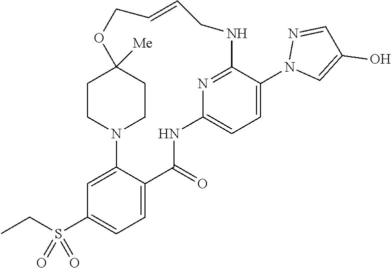 [SMILES: CCS(=O)(=O)c1ccc2c(c1)N1CCC(C)(CC1)OC/C=C/CNc1nc(ccc1-n1cc(O)cn1)NC2=O] | 553.3 | |
| 264 | 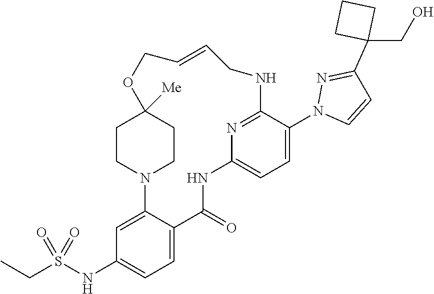 [SMILES: CCS(=O)(=O)Nc1ccc2c(c1)N1CCC(C)(CC1)OC/C=C/CNc1nc(ccc1-n1ccc(C3(CO)CCC3)n1)NC2=O] | 636.2 | 636.294 |
| 265 | 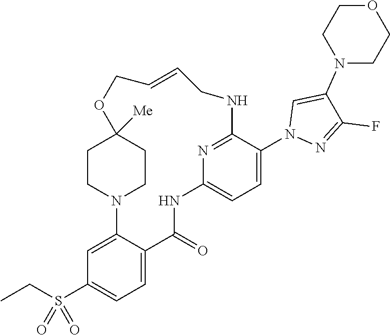 [SMILES: CCS(=O)(=O)c1ccc2c(c1)N1CCC(C)(CC1)OC/C=C/CNc1nc(ccc1-n1cc(N3CCOCC3)c(F)n1)NC2=O] | 640.3 | 640.2697 |
| 266 | 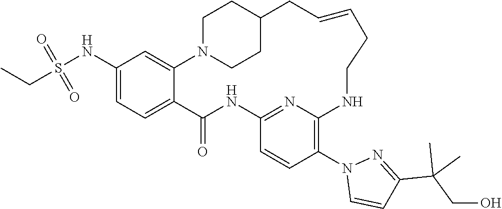 [SMILES: CCS(=O)(=O)Nc1ccc2c(c1)N1CCC(C/C=C/CCNc3nc(ccc3-n3ccc(C(C)(C)CO)n3)NC2=O)CC1] | 608.2 | 608.3001 |
| 267 | 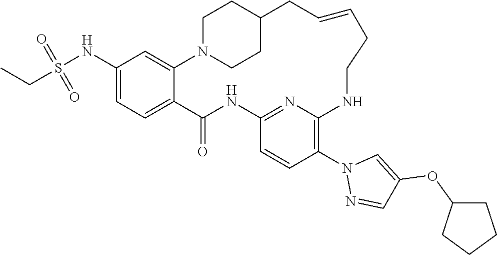 [SMILES: CCS(=O)(=O)Nc1ccc2c(c1)N1CCC(C/C=C/CCNc3nc(ccc3-n3cc(OC4CCCC4)cn3)NC2=O)CC1] | 620.2 | 620.2994 |
| 268 | 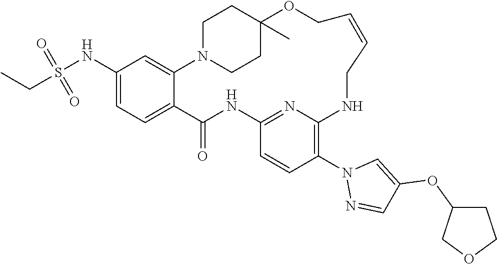 [SMILES: CCS(=O)(=O)Nc1ccc2c(c1)N1CCC(C)(CC1)OC/C=C\CNc1nc(ccc1-n1cc(OC3CCOC3)cn1)NC2=O] | 638.2 | 638.2749 |
| 269 | 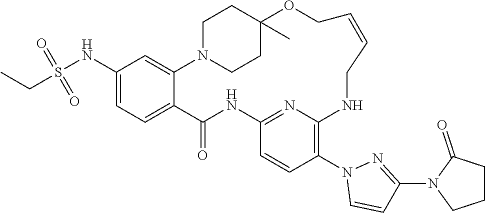 [SMILES: CCS(=O)(=O)Nc1ccc2c(c1)N1CCC(C)(CC1)OC/C=C\CNc1nc(ccc1-n1ccc(N3CCCC3=O)n1)NC2=O] | 635.2 | 635.2738 |
| 270 | 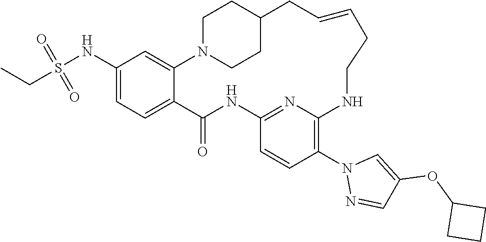 [SMILES: CCS(=O)(=O)Nc1ccc2c(c1)N1CCC(C/C=C/CCNc3nc(ccc3-n3cc(OC4CCC4)cn3)NC2=O)CC1] | 606.2 | 606.2851 |
| 271 | 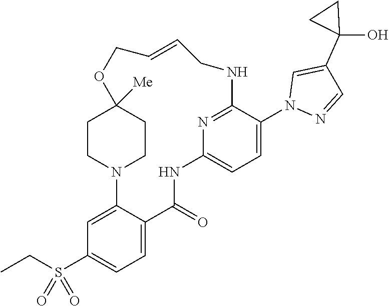 [SMILES: CCS(=O)(=O)c1ccc2c(c1)N1CCC(C)(CC1)OC/C=C/CNc1nc(ccc1-n1cc(C3(O)CC3)cn1)NC2=O] | 593.3 | 593.252 |
| 272 | 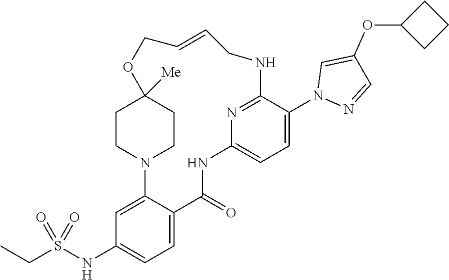 [SMILES: CCS(=O)(=O)Nc1ccc2c(c1)N1CCC(C)(CC1)OC/C=C/CNc1nc(ccc1-n1cc(OC3CCC3)cn1)NC2=O] | 622.2 | 622.279 |
| 273 | 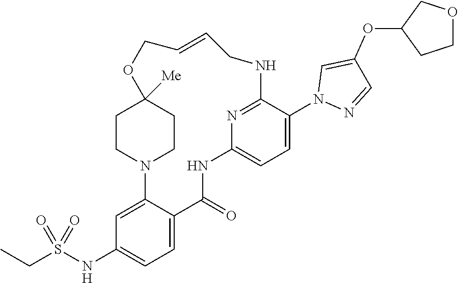 [SMILES: CCS(=O)(=O)Nc1ccc2c(c1)N1CCC(C)(CC1)OC/C=C/CNc1nc(ccc1-n1cc(OC3CCOC3)cn1)NC2=O] | 638.2 | 638.2749 |
| 274 | 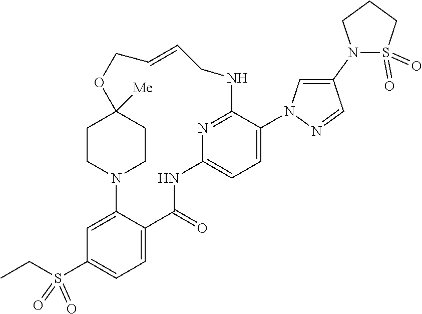 [SMILES: CCS(=O)(=O)c1ccc2c(c1)N1CCC(C)(CC1)OC/C=C/CNc1nc(ccc1-n1cc(N3CCCS3(=O)=O)cn1)NC2=O] | 656.3 | 656.2316 |
| 275 | 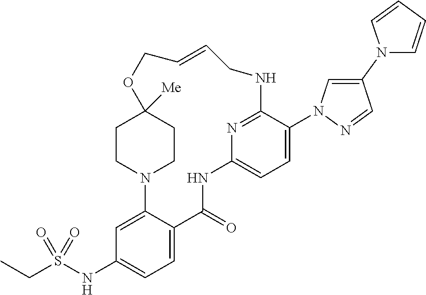 [SMILES: CCS(=O)(=O)Nc1ccc2c(c1)N1CCC(C)(CC1)OC/C=C/CNc1nc(ccc1-n1cc(-n3cccc3)cn1)NC2=O] | 617.1 | 617.263 |
| 276 | 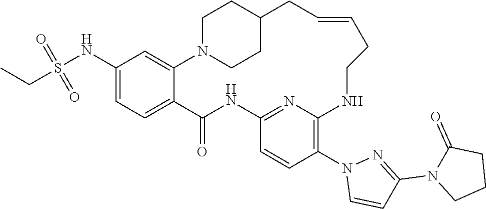 [SMILES: CCS(=O)(=O)Nc1ccc2c(c1)N1CCC(C/C=C/CCNc3nc(ccc3-n3ccc(N4CCCC4=O)n3)NC2=O)CC1] | 619.2 | 619.2795 |
| 277 | 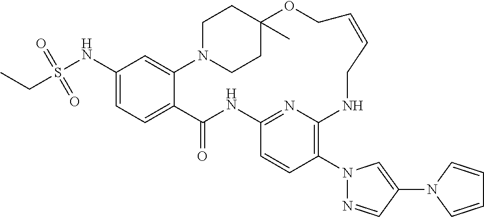 [SMILES: CCS(=O)(=O)Nc1ccc2c(c1)N1CCC(C)(CC1)OC/C=C\CNc1nc(ccc1-n1cc(-n3cccc3)cn1)NC2=O] | 617.2 | 617.2638 |
| 278 | 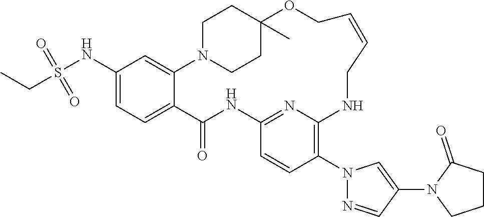 [SMILES: CCS(=O)(=O)Nc1ccc2c(c1)N1CCC(C)(CC1)OC/C=C\CNc1nc(ccc1-n1cc(N3CCCC3=O)cn1)NC2=O] | 635.2 | 635.2745 |
| 279 | 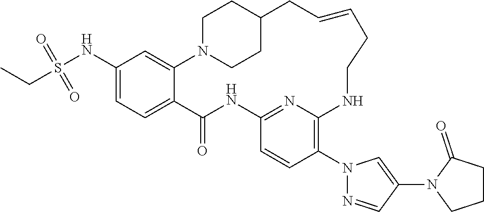 [SMILES: CCS(=O)(=O)Nc1ccc2c(c1)N1CCC(C/C=C/CCNc3nc(ccc3-n3cc(N4CCCC4=O)cn3)NC2=O)CC1] | 619.1 | 619.2799 |
| 280 | 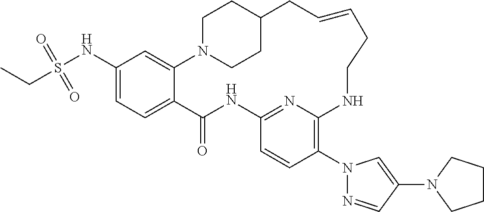 [SMILES: CCS(=O)(=O)Nc1ccc2c(c1)N1CCC(C/C=C/CCNc3nc(ccc3-n3cc(N4CCCC4)cn3)NC2=O)CC1] | 605.3 | 605.2996 |
| 281 | 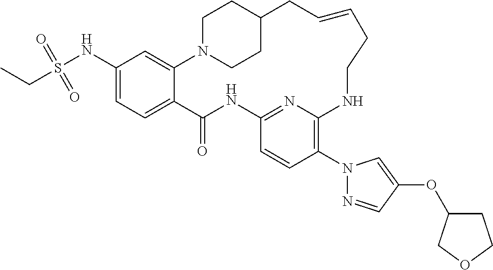 [SMILES: CCS(=O)(=O)Nc1ccc2c(c1)N1CCC(C/C=C/CCNc3nc(ccc3-n3cc(OC4CCOC4)cn3)NC2=O)CC1] | 622.2 | 622.2789 |
| 282 | 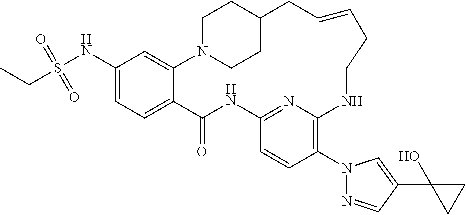 [SMILES: CCS(=O)(=O)Nc1ccc2c(c1)N1CCC(C/C=C/CCNc3nc(ccc3-n3cc(C4(O)CC4)cn3)NC2=O)CC1] | | 592.2689 |
| 283 | 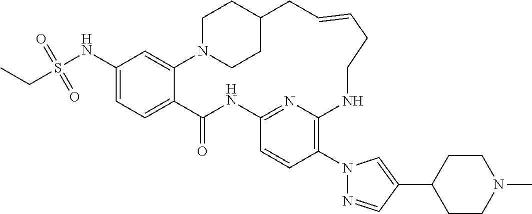 [SMILES: CCS(=O)(=O)Nc1ccc2c(c1)N1CCC(C/C=C/CCNc3nc(ccc3-n3cc(C4CCN(C)CC4)cn3)NC2=O)CC1] | 633.2 | 633.3314 |
| 284 | 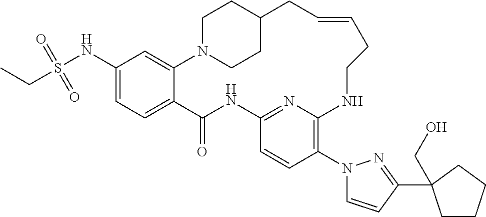 [SMILES: CCS(=O)(=O)Nc1ccc2c(c1)N1CCC(C/C=C/CCNc3nc(ccc3-n3ccc(C4(CO)CCCC4)n3)NC2=O)CC1] | 634.3 | 634.3152 |
| 285 | 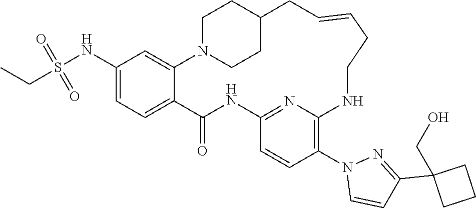 [SMILES: CCS(=O)(=O)Nc1ccc2c(c1)N1CCC(C/C=C/CCNc3nc(ccc3-n3ccc(C4(CO)CCC4)n3)NC2=O)CC1] | 620.3 | 620.3004 |
| 286 | 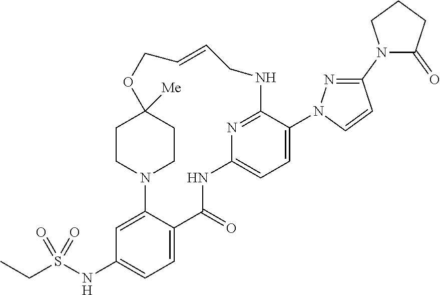 [SMILES: CCS(=O)(=O)Nc1ccc2c(c1)N1CCC(C)(CC1)OC/C=C/CNc1nc(ccc1-n1ccc(N3CCCC3=O)n1)NC2=O] | 635.1 | 635.2753 |
| 287 | 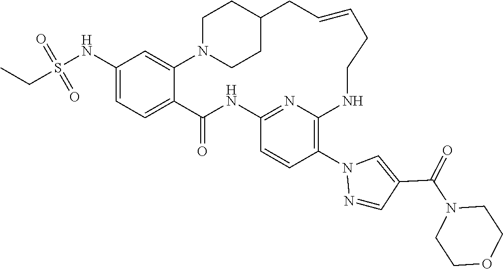 [SMILES: CCS(=O)(=O)Nc1ccc2c(c1)N1CCC(C/C=C/CCNc3nc(ccc3-n3cc(C(=O)N4CCOCC4)cn3)NC2=O)CC1] | 649.2 | |
| 288 | 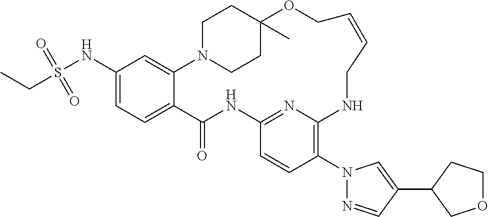 [SMILES: CCS(=O)(=O)Nc1ccc2c(c1)N1CCC(C)(CC1)OC/C=C\CNc1nc(ccc1-n1cc(C3CCOC3)cn1)NC2=O] | 606.2 | 606.2851 |
| 289 | 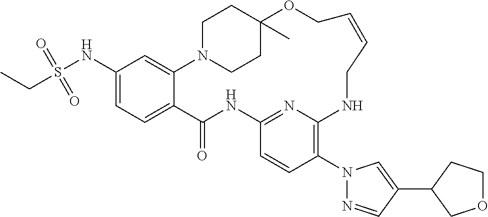 [SMILES: CCS(=O)(=O)Nc1ccc2c(c1)N1CCC(C)(CC1)OC/C=C\CNc1nc(ccc1-n1cc(C3CCOC3)cn1)NC2=O] | 622.2 | 622.2799 |
| 290 | 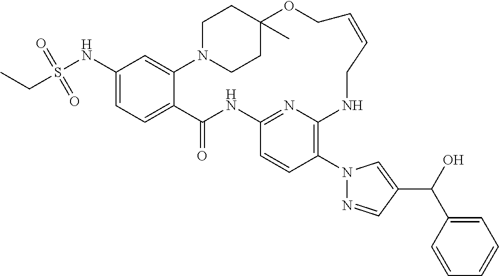 [SMILES: CCS(=O)(=O)Nc1ccc2c(c1)N1CCC(C)(CC1)OC/C=C\CNc1nc(ccc1-n1cc(C(O)c3ccccc3)cn1)NC2=O] | 658.2 | 658.2796 |
| 291 | 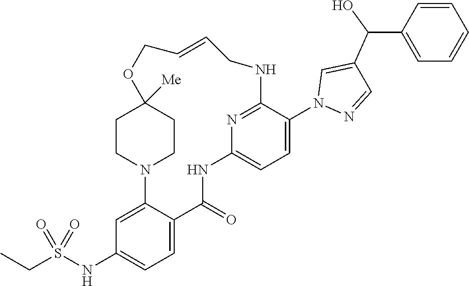 [SMILES: CCS(=O)(=O)Nc1ccc2c(c1)N1CCC(C)(CC1)OC/C=C/CNc1nc(ccc1-n1cc(C(O)c3ccccc3)cn1)NC2=O] | 658.2 | 658.2799 |
| 292 | 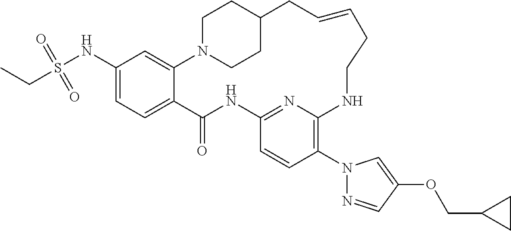 [SMILES: CCS(=O)(=O)Nc1ccc2c(c1)N1CCC(C/C=C/CCNc3nc(ccc3-n3cc(OCC4CC4)cn3)NC2=O)CC1] | 606.2 | 606.2843 |
| 293 | 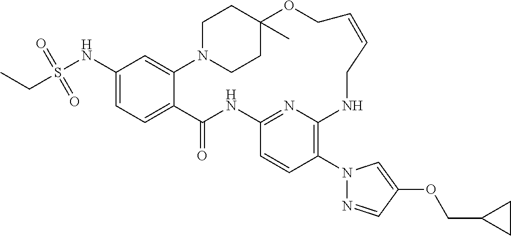 [SMILES: CCS(=O)(=O)Nc1ccc2c(c1)N1CCC(C)(CC1)OC/C=C\CNc1nc(ccc1-n1cc(OCC3CC3)cn1)NC2=O] | 622.3 | 622.2799 |
| 294 | 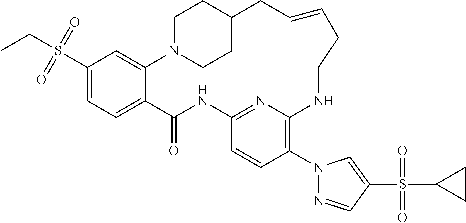 [SMILES: CCS(=O)(=O)c1ccc2c(c1)N1CCC(C/C=C/CCNc3nc(ccc3-n3cc(S(=O)(=O)C4CC4)cn3)NC2=O)CC1] | 625.2 | 625.2246 |
| 295 | 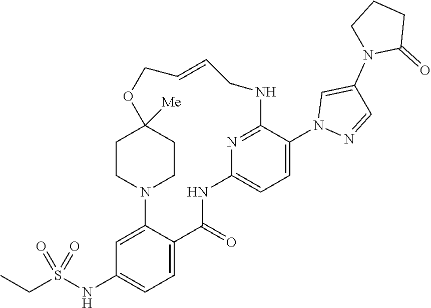 [SMILES: CCS(=O)(=O)Nc1ccc2c(c1)N1CCC(C)(CC1)OC/C=C/CNc1nc(ccc1-n1cc(N3CCCC3=O)cn1)NC2=O] | 635.1 | 635.2745 |
| 296 | 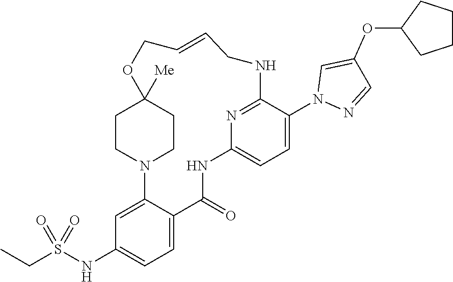 [SMILES: CCS(=O)(=O)Nc1ccc2c(c1)N1CCC(C)(CC1)OC/C=C/CNc1nc(ccc1-n1cc(OC3CCCC3)cn1)NC2=O] | 636.2 | 636.2954 |
| 297 | 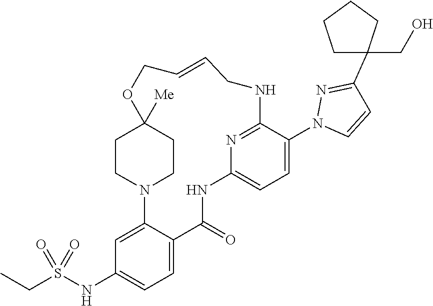 [SMILES: CCS(=O)(=O)Nc1ccc2c(c1)N1CCC(C)(CC1)OC/C=C/CNc1nc(ccc1-n1ccc(C3(CO)CCCC3)n1)NC2=O] | 650.2 | 650.3096 |
| 298 | 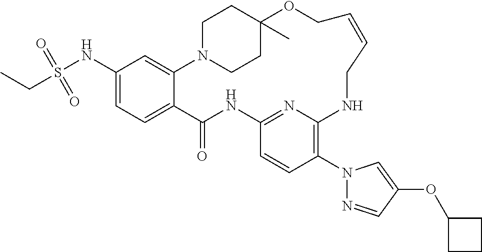 [SMILES: CCS(=O)(=O)Nc1ccc2c(c1)N1CCC(C)(CC1)OC/C=C\CNc1nc(ccc1-n1cc(OC3CCC3)cn1)NC2=O] | 622.2 | 622.2793 |
| 299 | 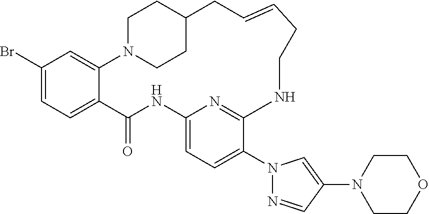 [SMILES: O=C1Nc2ccc(-n3cc(N4CCOCC4)cn3)c(n2)NCC/C=C/CC2CCN(CC2)c2cc(Br)ccc21] | 592.2 | 592.2013 |
| 300 | 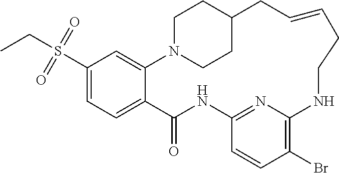 [SMILES: CCS(=O)(=O)c1ccc2c(c1)N1CCC(C/C=C/CCNc3nc(ccc3Br)NC2=O)CC1] | | 533.1212 |
| 301 | 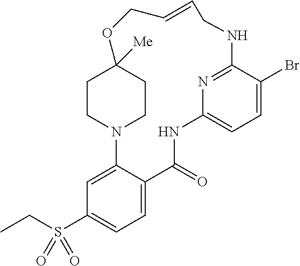 [SMILES: CCS(=O)(=O)c1ccc2c(c1)N1CCC(C)(CC1)OC/C=C/CNc1nc(ccc1Br)NC2=O] | 549.0 | 549.1145 |
| 302 | 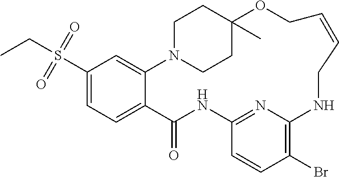 [SMILES: CCS(=O)(=O)c1ccc2c(c1)N1CCC(C)(CC1)OC/C=C\CNc1nc(ccc1Br)NC2=O] | 549.0 | 549.1154 |
| 303 | 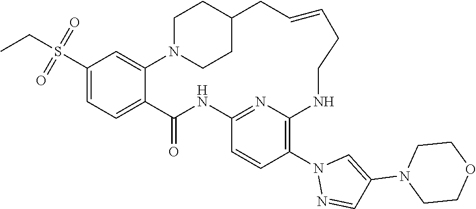 [SMILES: CCS(=O)(=O)c1ccc2c(c1)N1CCC(C/C=C/CCNc3nc(ccc3-n3cc(N4CCOCC4)cn3)NC2=O)CC1] | 606.3 | 606.2835 |
| 304 | 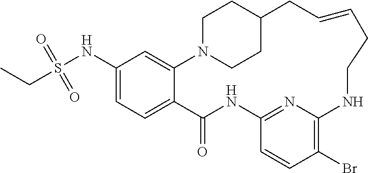 [SMILES: CCS(=O)(=O)Nc1ccc2c(c1)N1CCC(C/C=C/CCNc3nc(ccc3Br)NC2=O)CC1] | 548.2 | 548.1305 |
| 305 | 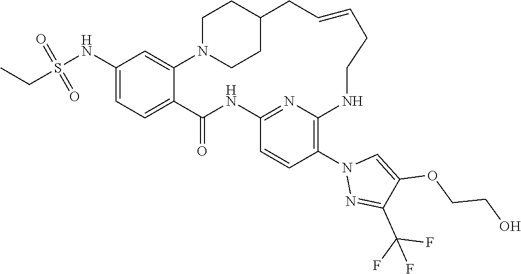 [SMILES: CCS(=O)(=O)Nc1ccc2c(c1)N1CCC(C/C=C/CCNc3nc(ccc3-n3cc(OCCO)c(C(F)(F)F)n3)NC2=O)CC1] | 664.2 | 664.2501 |
| 306 | 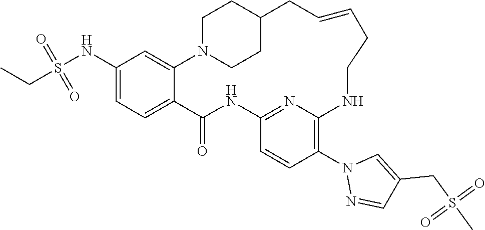 [SMILES: CCS(=O)(=O)Nc1ccc2c(c1)N1CCC(C/C=C/CCNc3nc(ccc3-n3cc(CS(C)(=O)=O)cn3)NC2=O)CC1] | 628.1 | 628.2359 |
| 307 | 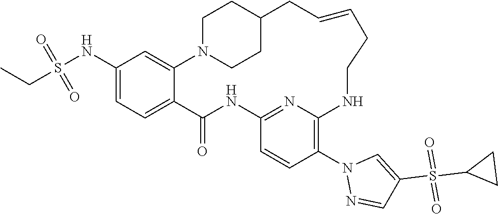 [SMILES: CCS(=O)(=O)Nc1ccc2c(c1)N1CCC(C/C=C/CCNc3nc(ccc3-n3cc(S(=O)(=O)C4CC4)cn3)NC2=O)CC1] | 640.1 | 640.2356 |
| 308 | 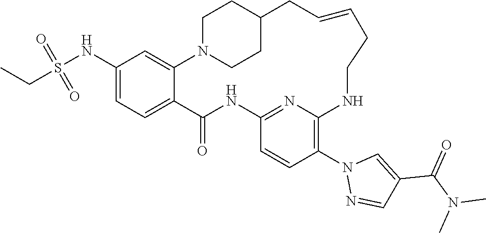 [SMILES: CCS(=O)(=O)Nc1ccc2c(c1)N1CCC(C/C=C/CCNc3nc(ccc3-n3cc(C(=O)N(C)C)cn3)NC2=O)CC1] | 607.2 | 607.2799 |
| 309 | 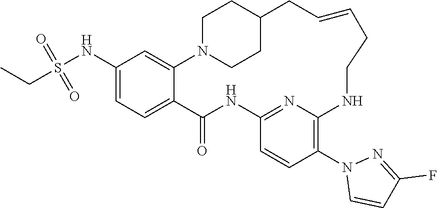 [SMILES: CCS(=O)(=O)Nc1ccc2c(c1)N1CCC(C/C=C/CCNc3nc(ccc3-n3ccc(F)n3)NC2=O)CC1] | 554.1 | 554.233 |
| 310 | 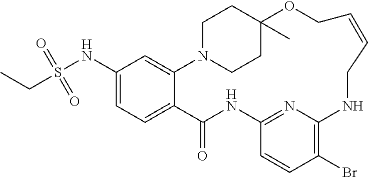 [SMILES: CCS(=O)(=O)Nc1ccc2c(c1)N1CCC(C)(CC1)OC/C=C\CNc1nc(ccc1Br)NC2=O] | 564.0 | 564.1261 |
| 311 | 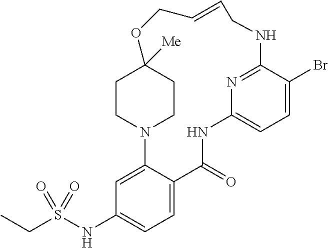 [SMILES: CCS(=O)(=O)Nc1ccc2c(c1)N1CCC(C)(CC1)OC/C=C/CNc1nc(ccc1Br)NC2=O] | 564.0 | 564.1264 |
| 312 | 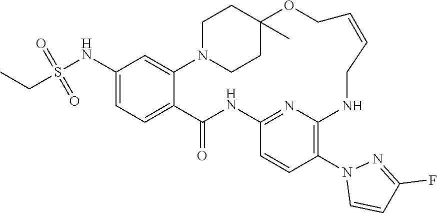 [SMILES: CCS(=O)(=O)Nc1ccc2c(c1)N1CCC(C)(CC1)OC/C=C\CNc1nc(ccc1-n1ccc(F)n1)NC2=O] | 570.1 | 570.2283 |
| 313 | 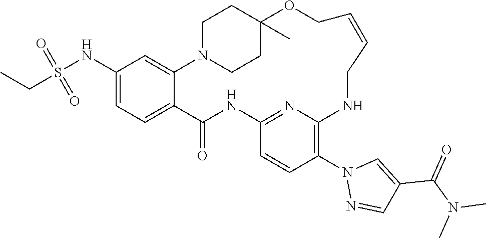 [SMILES: CCS(=O)(=O)Nc1ccc2c(c1)N1CCC(C)(CC1)OC/C=C\CNc1nc(ccc1-n1cc(C(=O)N(C)C)cn1)NC2=O] | 623.1 | 623.2734 |
| 314 | 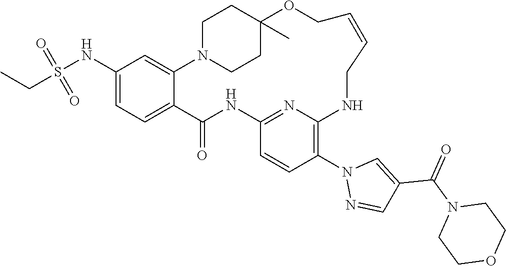 [SMILES: CCS(=O)(=O)Nc1ccc2c(c1)N1CCC(C)(CC1)OC/C=C\CNc1nc(ccc1-n1cc(C(=O)N3CCOCC3)cn1)NC2=O] | 665.2 | 665.2851 |
| 315 | 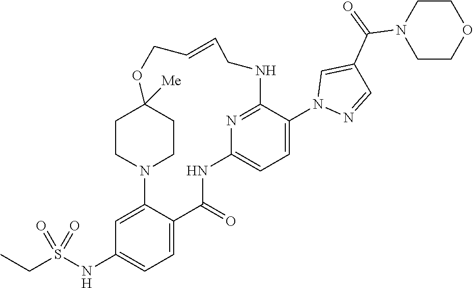 [SMILES: CCS(=O)(=O)Nc1ccc2c(c1)N1CCC(C)(CC1)OC/C=C/CNc1nc(ccc1-n1cc(C(=O)N3CCOCC3)cn1)NC2=O] | 665.2 | 665.2845 |
| 316 | 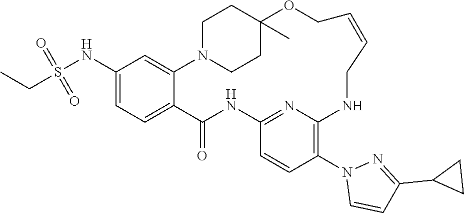 [SMILES: CCS(=O)(=O)Nc1ccc2c(c1)N1CCC(C)(CC1)OC/C=C\CNc1nc(ccc1-n1ccc(C3CC3)n1)NC2=O] | 592.2 | 592.2687 |
| 317 | 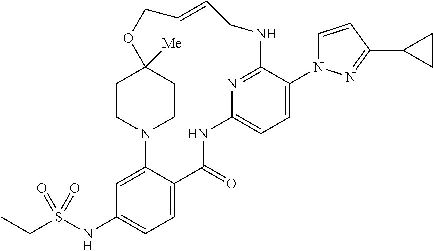 [SMILES: CCS(=O)(=O)Nc1ccc2c(c1)N1CCC(C)(CC1)OC/C=C/CNc1nc(ccc1-n1ccc(C3CC3)n1)NC2=O] | 592.1 | 592.2683 |
| 318 | 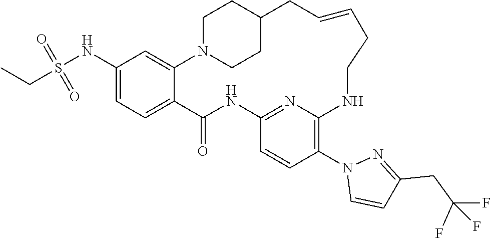 [SMILES: CCS(=O)(=O)Nc1ccc2c(c1)N1CCC(C/C=C/CCNc3nc(ccc3-n3ccc(CC(F)(F)F)n3)NC2=O)CC1] | 618.2 | 618.2454 |
| 319 | 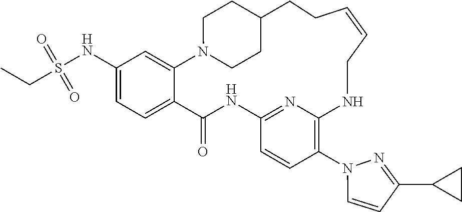 [SMILES: CCS(=O)(=O)Nc1ccc2c(c1)N1CCC(CC/C=C\CNc3nc(ccc3-n3ccc(C4CC4)n3)NC2=O)CC1] | 576.2 | 576.2743 |
| 320 | 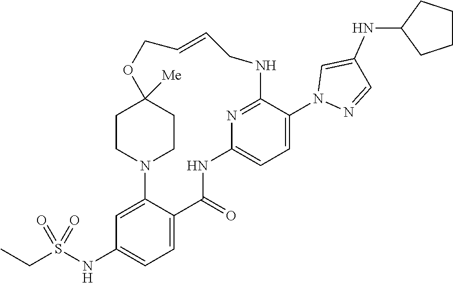 [SMILES: CCS(=O)(=O)Nc1ccc2c(c1)N1CCC(C)(CC1)OC/C=C/CNc1nc(ccc1-n1cc(NC3CCCC3)cn1)NC2=O] | 635.3 | 635.31 |
| 321 | 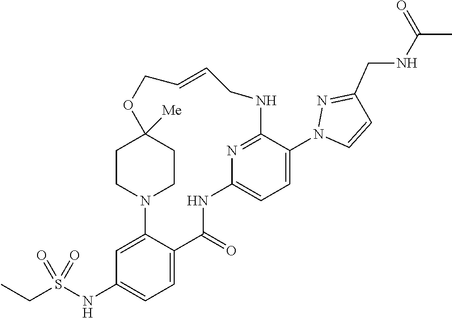 [SMILES: CCS(=O)(=O)Nc1ccc2c(c1)N1CCC(C)(CC1)OC/C=C/CNc1nc(ccc1-n1ccc(CNC(C)=O)n1)NC2=O] | 623.2 | 623.2754 |
| 322 | 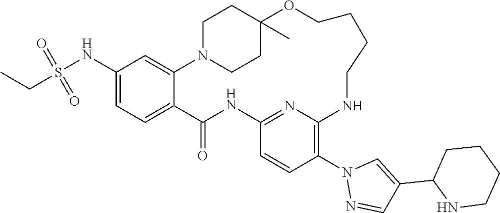 [SMILES: CCS(=O)(=O)Nc1ccc2c(c1)N1CCC(C)(CC1)OCCCCNc1nc(ccc1-n1cc(C3CCCCN3)cn1)NC2=O] | 635.2 | 635.3105 |
| 323 | 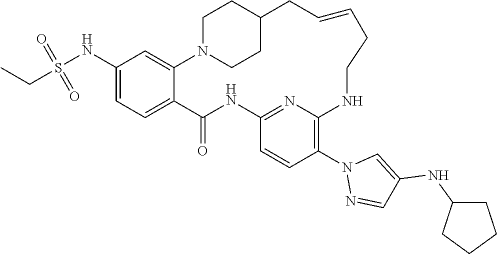 [SMILES: CCS(=O)(=O)Nc1ccc2c(c1)N1CCC(C/C=C/CCNc3nc(ccc3-n3cc(NC4CCCC4)cn3)NC2=O)CC1] | 619.2 | 619.3149 |
| 324 | 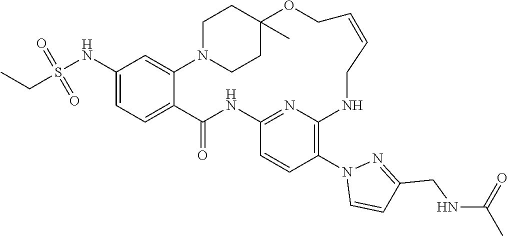 [SMILES: CCS(=O)(=O)Nc1ccc2c(c1)N1CCC(C)(CC1)OC/C=C\CNc1nc(ccc1-n1ccc(CNC(C)=O)n1)NC2=O] | | 623.2738 |
| 325 | 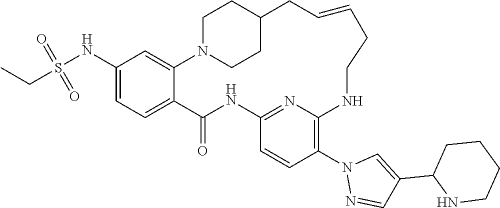 [SMILES: CCS(=O)(=O)Nc1ccc2c(c1)N1CCC(C/C=C/CCNc3nc(ccc3-n3cc(C4CCCCN4)cn3)NC2=O)CC1] | 619.2 | 619.3152 |
| 326 | 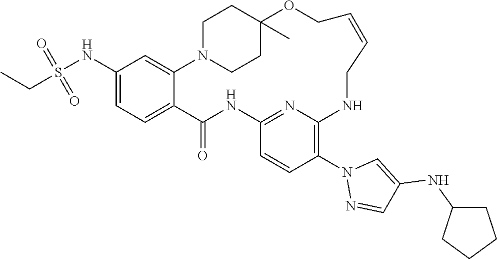 [SMILES: CCS(=O)(=O)Nc1ccc2c(c1)N1CCC(C)(CC1)OC/C=C\CNc1nc(ccc1-n1cc(NC3CCCC3)cn1)NC2=O] | 635.2 | 635.3101 |
| 327 | 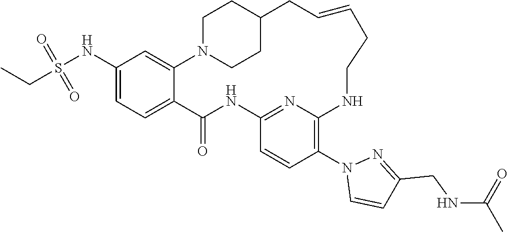 [SMILES: CCS(=O)(=O)Nc1ccc2c(c1)N1CCC(C/C=C/CCNc3nc(ccc3-n3ccc(CNC(C)=O)n3)NC2=O)CC1] | 607.2 | 607.2794 |
| 328 | 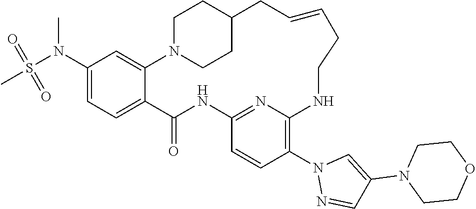 [SMILES: CN(c1ccc2c(c1)N1CCC(C/C=C/CCNc3nc(ccc3-n3cc(N4CCOCC4)cn3)NC2=O)CC1)S(C)(=O)=O] | 621.2 | 621.2947 |
| 329 | 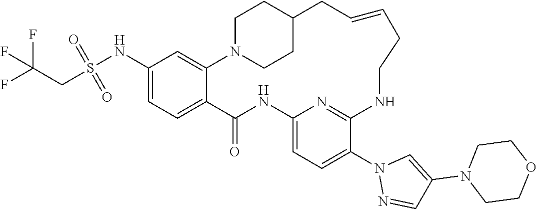 [SMILES: O=C1Nc2ccc(-n3cc(N4CCOCC4)cn3)c(n2)NCC/C=C/CC2CCN(CC2)c2cc(NS(=O)(=O)CC(F)(F)F)ccc21] | 675.2 | 675.267 |
| 330 | 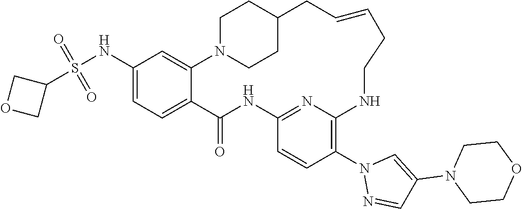 [SMILES: O=C1Nc2ccc(-n3cc(N4CCOCC4)cn3)c(n2)NCC/C=C/CC2CCN(CC2)c2cc(NS(=O)(=O)C3COC3)ccc21] | 649.2 | 649.2902 |
| 331 | 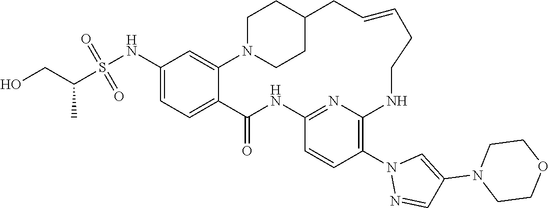 [SMILES: C[C@H](CO)S(=O)(=O)Nc1ccc2c(c1)N1CCC(C/C=C/CCNc3nc(ccc3-n3cc(N4CCOCC4)cn3)NC2=O)CC1] | 651.3 | 651.306 |
| 332 | 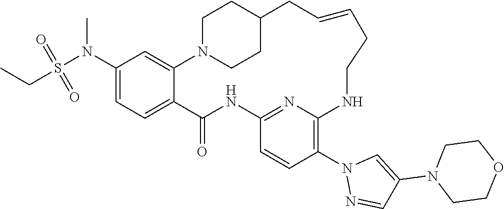 [SMILES: CCS(=O)(=O)N(C)c1ccc2c(c1)N1CCC(C/C=C/CCNc3nc(ccc3-n3cc(N4CCOCC4)cn3)NC2=O)CC1] | 635.2 | 635.3105 |
| 333 | 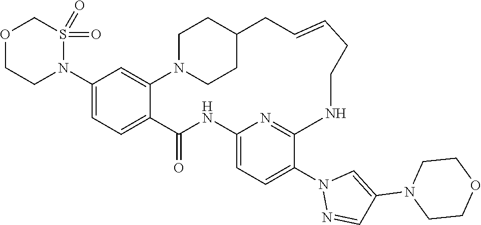 [SMILES: O=C1Nc2ccc(-n3cc(N4CCOCC4)cn3)c(n2)NCC/C=C/CC2CCN(CC2)c2cc(N3CCOCS3(=O)=O)ccc21] | 649.2 | 649.2897 |
| 334 | 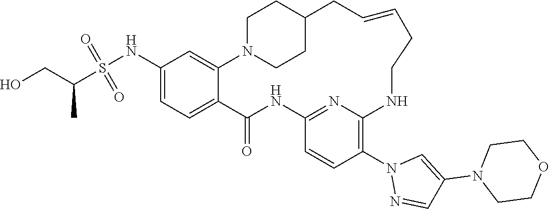 [SMILES: C[C@@H](CO)S(=O)(=O)Nc1ccc2c(c1)N1CCC(C/C=C/CCNc3nc(ccc3-n3cc(N4CCOCC4)cn3)NC2=O)CC1] | 651.2 | 651.306 |
| 335 | 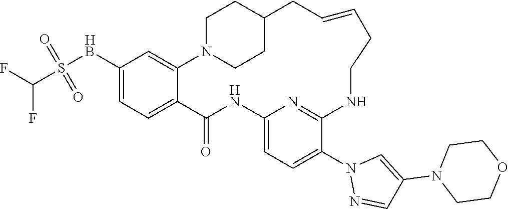 | 643.2 | 643.2608 |
| 336 | 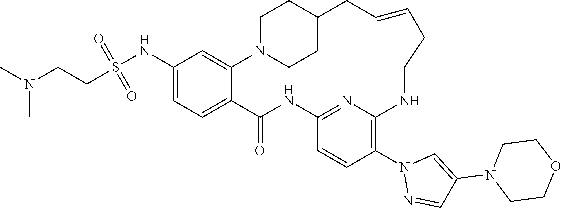 [SMILES: CN(C)CCS(=O)(=O)Nc1ccc2c(c1)N1CCC(C/C=C/CCNc3nc(ccc3-n3cc(N4CCOCC4)cn3)NC2=O)CC1] | 664.3 | 664.3363 |
| 337 | 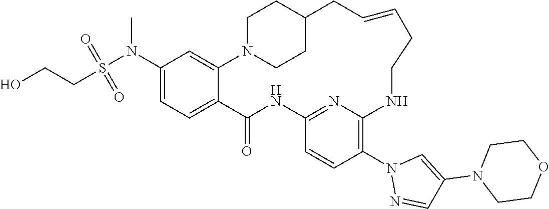 [SMILES: CN(c1ccc2c(c1)N1CCC(C/C=C/CCNc3nc(ccc3-n3cc(N4CCOCC4)cn3)NC2=O)CC1)S(=O)(=O)CCO] | 651.2 | 651.304 |
| 338 | 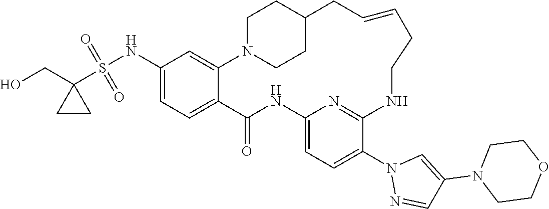 [SMILES: O=C1Nc2ccc(-n3cc(N4CCOCC4)cn3)c(n2)NCC/C=C/CC2CCN(CC2)c2cc(NS(=O)(=O)C3(CO)CC3)ccc21] | 663.2 | 663.305 |
| 339 | 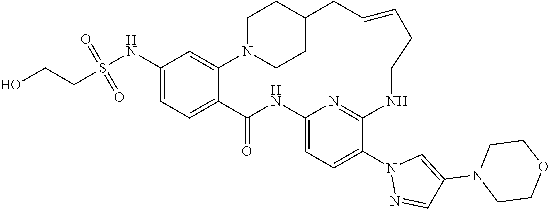 [SMILES: O=C1Nc2ccc(-n3cc(N4CCOCC4)cn3)c(n2)NCC/C=C/CC2CCN(CC2)c2cc(NS(=O)(=O)CCO)ccc21] | 637.2 | 637.2903 |
| 340 | 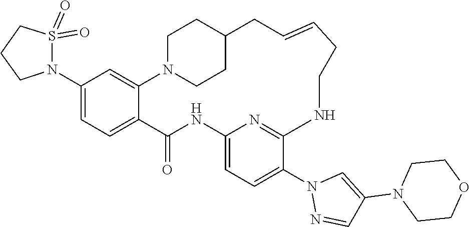 [SMILES: O=C1Nc2ccc(-n3cc(N4CCOCC4)cn3)c(n2)NCC/C=C/CC2CCN(CC2)c2cc(N3CCCS3(=O)=O)ccc21] | 633.2 | 633.2955 |
| 341 | 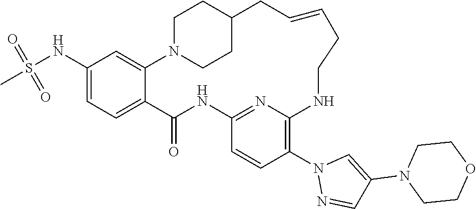 [SMILES: CS(=O)(=O)Nc1ccc2c(c1)N1CCC(C/C=C/CCNc3nc(ccc3-n3cc(N4CCOCC4)cn3)NC2=O)CC1] | 607.2 | 607.2794 |
| 342 | 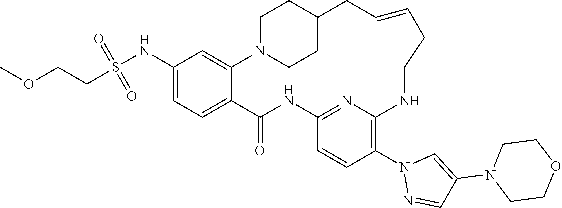 [SMILES: COCCS(=O)(=O)Nc1ccc2c(c1)N1CCC(C/C=C/CCNc3nc(ccc3-n3cc(N4CCOCC4)cn3)NC2=O)CC1] | 651.2 | 651.3052 |
| 343 | 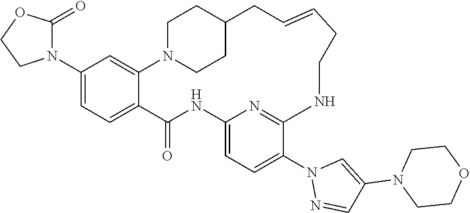 [SMILES: O=C1Nc2ccc(-n3cc(N4CCOCC4)cn3)c(n2)NCC/C=C/CC2CCN(CC2)c2cc(N3CCOC3=O)ccc21] | 599.2 | 599.3073 |
| 344 | 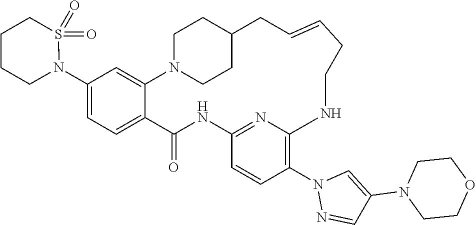 [SMILES: O=C1Nc2ccc(-n3cc(N4CCOCC4)cn3)c(n2)NCC/C=C/CC2CCN(CC2)c2cc(N3CCCCS3(=O)=O)ccc21] | 647.2 | 647.3111 |
| 345 | 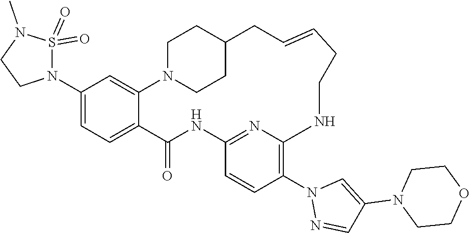 [SMILES: CN1CCN(c2ccc3c(c2)N2CCC(C/C=C/CCNc4nc(ccc4-n4cc(N5CCOCC5)cn4)NC3=O)CC2)S1(=O)=O] | 648.3 | 648.3063 |
| 346 | 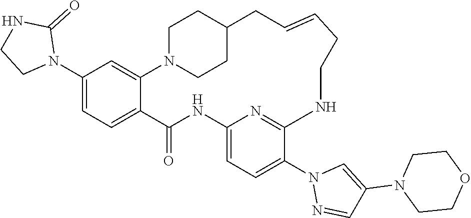 [SMILES: O=C1Nc2ccc(-n3cc(N4CCOCC4)cn3)c(n2)NCC/C=C/CC2CCN(CC2)c2cc(N3CCNC3=O)ccc21] | 598.2 | 598.3227 |
| 347 | 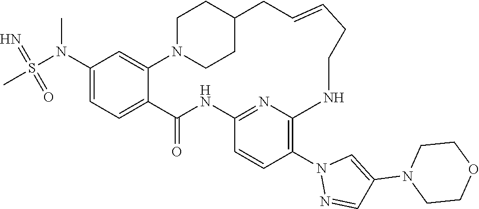 [SMILES: CN(c1ccc2c(c1)N1CCC(C/C=C/CCNc3nc(ccc3-n3cc(N4CCOCC4)cn3)NC2=O)CC1)S(C)(=N)=O] | 620.2 | 620.311 |
| 348 | 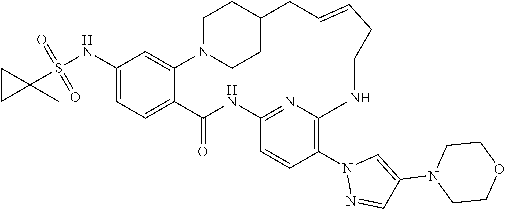 [SMILES: CC1(S(=O)(=O)Nc2ccc3c(c2)N2CCC(C/C=C/CCNc4nc(ccc4-n4cc(N5CCOCC5)cn4)NC3=O)CC2)CC1] | 647.3 | 647.3108 |
| 349 | 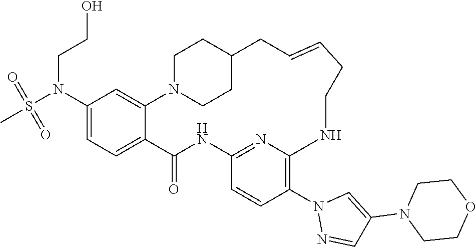 [SMILES: CS(=O)(=O)N(CCO)c1ccc2c(c1)N1CCC(C/C=C/CCNc3nc(ccc3-n3cc(N4CCOCC4)cn3)NC2=O)CC1] | 651.2 | 651.3058 |
| 350 | 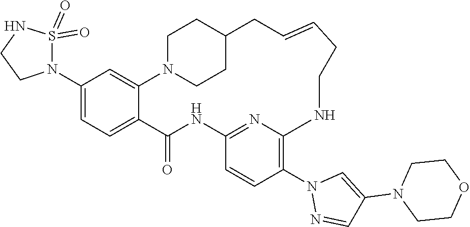 [SMILES: O=C1Nc2ccc(-n3cc(N4CCOCC4)cn3)c(n2)NCC/C=C/CC2CCN(CC2)c2cc(N3CCNS3(=O)=O)ccc21] | 634.2 | 634.291 |
| 351 | 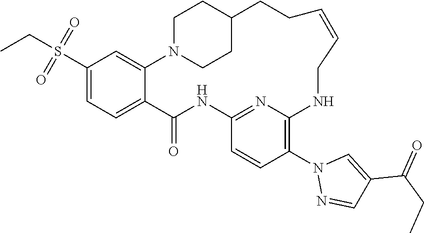 [SMILES: CCC(=O)c1cnn(-c2ccc3nc2NC/C=C\CCC2CCN(CC2)c2cc(S(=O)(=O)CC)ccc2C(=O)N3)c1] | 577.3 | 577.2576 |
| 352 | 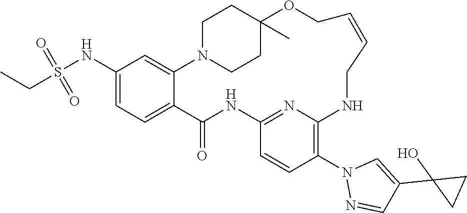 [SMILES: CCS(=O)(=O)Nc1ccc2c(c1)N1CCC(C)(CC1)OC/C=C\CNc1nc(ccc1-n1cc(C3(O)CC3)cn1)NC2=O] | 608.4 | 608.2632 |
| 353 | 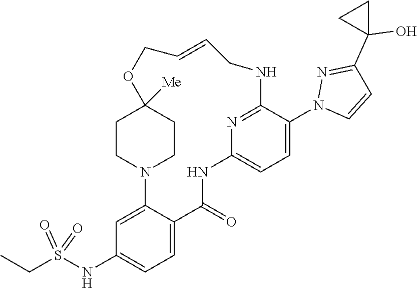 [SMILES: CCS(=O)(=O)Nc1ccc2c(c1)N1CCC(C)(CC1)OC/C=C/CNc1nc(ccc1-n1ccc(C3(O)CC3)n1)NC2=O] | 608.3 | 608.264 |
| 354 | 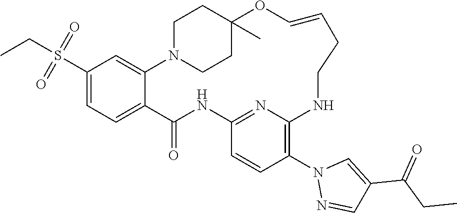 [SMILES: CCC(=O)c1cnn(-c2ccc3nc2NCC/C=C/OC2(C)CCN(CC2)c2cc(S(=O)(=O)CC)ccc2C(=O)N3)c1] | 593.3 | 593.253 |
| 355 | 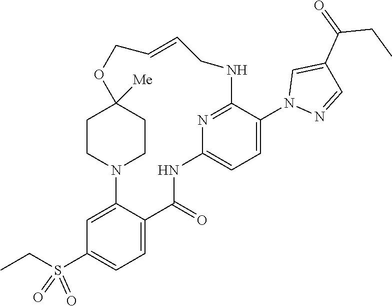 [SMILES: CCC(=O)c1cnn(-c2ccc3nc2NC/C=C/COC2(C)CCN(CC2)c2cc(S(=O)(=O)CC)ccc2C(=O)N3)c1] | 593.2 | 593.2519 |
| 356 | 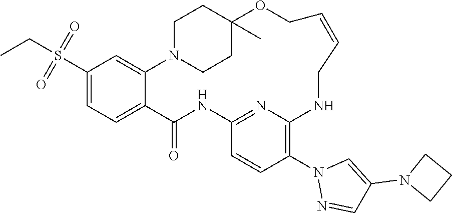 [SMILES: CCS(=O)(=O)c1ccc2c(c1)N1CCC(C)(CC1)OC/C=C\CNc1nc(ccc1-n1cc(N3CCC3)cn1)NC2=O] | 592.3 | 592.2672 |
| 357 | 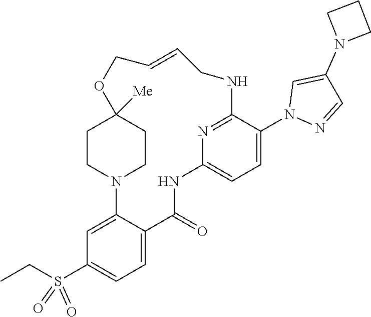 [SMILES: CCS(=O)(=O)c1ccc2c(c1)N1CCC(C)(CC1)OC/C=C/CNc1nc(ccc1-n1cc(N3CCC3)cn1)NC2=O] | 592.3 | 592.2676 |
| 358 | 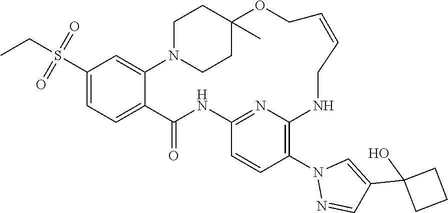 [SMILES: CCS(=O)(=O)c1ccc2c(c1)N1CCC(C)(CC1)OC/C=C\CNc1nc(ccc1-n1cc(C3(O)CCC3)cn1)NC2=O] | 607.3 | 607.2698 |
| 359 | 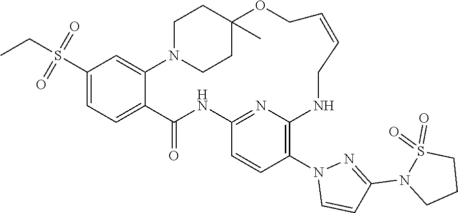 [SMILES: CCS(=O)(=O)c1ccc2c(c1)N1CCC(C)(CC1)OC/C=C\CNc1nc(ccc1-n1ccc(N3CCCS3(=O)=O)n1)NC2=O] | 656.3 | 656.2315 |
| 360 | 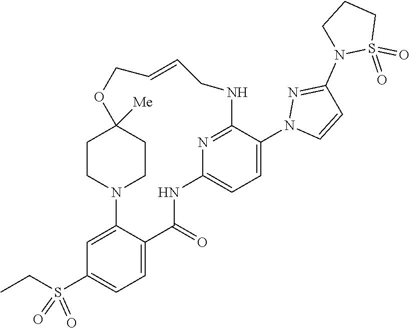 [SMILES: CCS(=O)(=O)c1ccc2c(c1)N1CCC(C)(CC1)OC/C=C/CNc1nc(ccc1-n1ccc(N3CCCS3(=O)=O)n1)NC2=O] | 656.3 | 656.2305 |
| 361 | 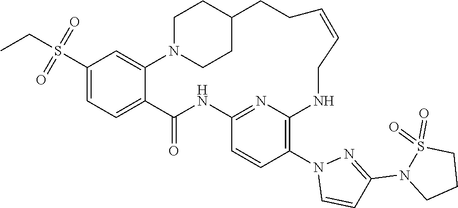 [SMILES: CCS(=O)(=O)c1ccc2c(c1)N1CCC(CC/C=C\CNc3nc(ccc3-n3ccc(N4CCCS4(=O)=O)n3)NC2=O)CC1] | 640.2 | 640.2361 |
| 362 | 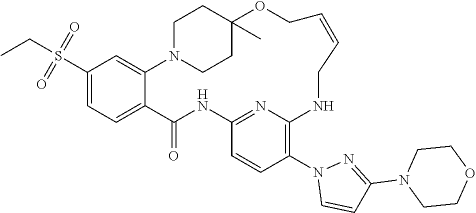 [SMILES: CCS(=O)(=O)c1ccc2c(c1)N1CCC(C)(CC1)OC/C=C\CNc1nc(ccc1-n1ccc(N3CCOCC3)n1)NC2=O] | 622.3 | 622.2791 |
| 363 | 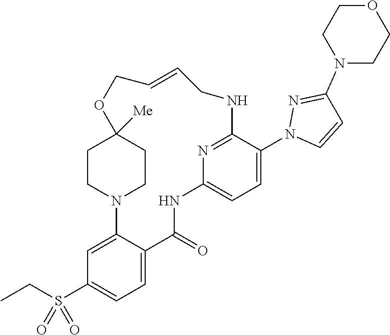 [SMILES: CCS(=O)(=O)c1ccc2c(c1)N1CCC(C)(CC1)OC/C=C/CNc1nc(ccc1-n1ccc(N3CCOCC3)n1)NC2=O] | 622.3 | 622.2791 |
| 364 | 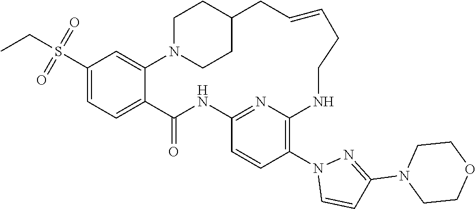 [SMILES: CCS(=O)(=O)c1ccc2c(c1)N1CCC(C/C=C/CCNc3nc(ccc3-n3ccc(N4CCOCC4)n3)NC2=O)CC1] | 606.3 | 606.2853 |
| 365 | 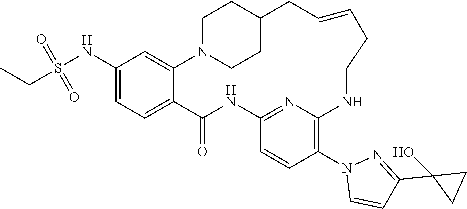 [SMILES: CCS(=O)(=O)Nc1ccc2c(c1)N1CCC(C/C=C/CCNc3nc(ccc3-n3ccc(C4(O)CC4)n3)NC2=O)CC1] | 592.3 | 592.2688 |
| 366 | 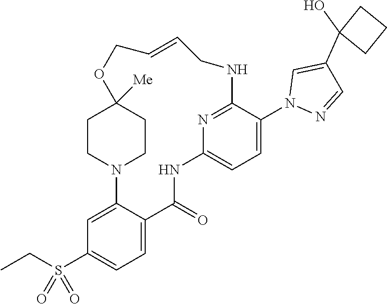 [SMILES: CCS(=O)(=O)c1ccc2c(c1)N1CCC(C)(CC1)OC/C=C/CNc1nc(ccc1-n1cc(C3(O)CCC3)cn1)NC2=O] | 607.3 | 607.2685 |
| 367 | 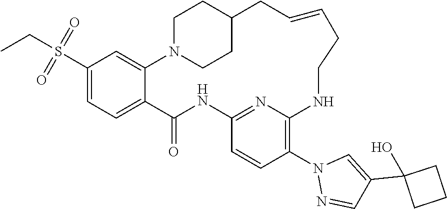 [SMILES: CCS(=O)(=O)c1ccc2c(c1)N1CCC(C/C=C/CCNc3nc(ccc3-n3cc(C4(O)CCC4)cn3)NC2=O)CC1] | 591.3 | 591.273 |
| 368 | 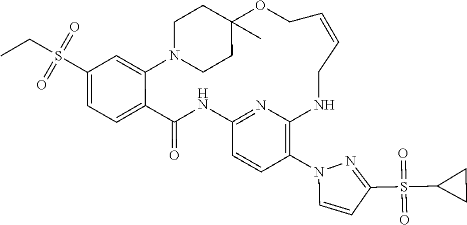 [SMILES: CCS(=O)(=O)c1ccc2c(c1)N1CCC(C)(CC1)OC/C=C\CNc1nc(ccc1-n1ccc(S(=O)(=O)C3CC3)n1)NC2=O] | 641.3 | 641.2202 |
| 369 | 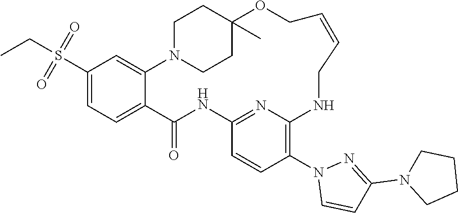 [SMILES: CCS(=O)(=O)c1ccc2c(c1)N1CCC(C)(CC1)OC/C=C\CNc1nc(ccc1-n1ccc(N3CCCC3)n1)NC2=O] | 606.3 | 606.2834 |
| 370 | 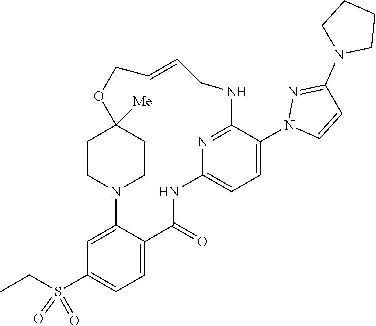 [SMILES: CCS(=O)(=O)c1ccc2c(c1)N1CCC(C)(CC1)OC/C=C/CNc1nc(ccc1-n1ccc(N3CCCC3)n1)NC2=O] | 606.4 | 606.2839 |
| 371 | 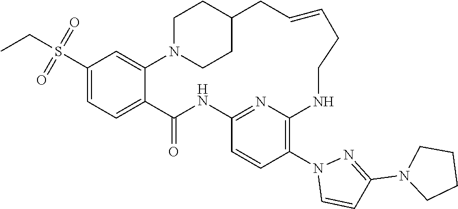 [SMILES: CCS(=O)(=O)c1ccc2c(c1)N1CCC(C/C=C/CCNc3nc(ccc3-n3ccc(N4CCCC4)n3)NC2=O)CC1] | | 590.2892 |
| 372 | 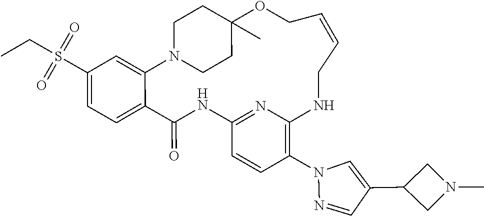 [SMILES: CCS(=O)(=O)c1ccc2c(c1)N1CCC(C)(CC1)OC/C=C\CNc1nc(ccc1-n1cc(C3CN(C)C3)cn1)NC2=O] | 606.3 | 606.2846 |
| 373 | 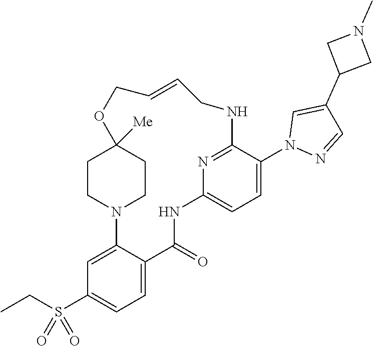 [SMILES: CCS(=O)(=O)c1ccc2c(c1)N1CCC(C)(CC1)OC/C=C/CNc1nc(ccc1-n1cc(C3CN(C)C3)cn1)NC2=O] | 606.3 | 606.2838 |
| 374 | 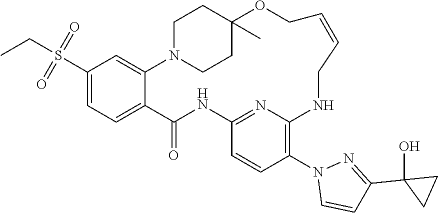 [SMILES: CCS(=O)(=O)c1ccc2c(c1)N1CCC(C)(CC1)OC/C=C\CNc1nc(ccc1-n1ccc(C3(O)CC3)n1)NC2=O] | 593.4 | 593.252 |
| 375 | 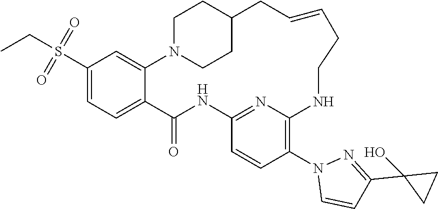 [SMILES: CCS(=O)(=O)c1ccc2c(c1)N1CCC(C/C=C/CCNc3nc(ccc3-n3ccc(C4(O)CC4)n3)NC2=O)CC1] | 577.4 | 577.2584 |
| 376 | 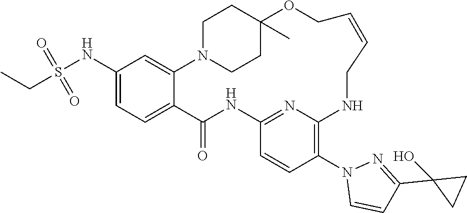 [SMILES: CCS(=O)(=O)Nc1ccc2c(c1)N1CCC(C)(CC1)OC/C=C\CNc1nc(ccc1-n1ccc(C3(O)CC3)n1)NC2=O] | 608.3 | 608.2637 |
| 377 | 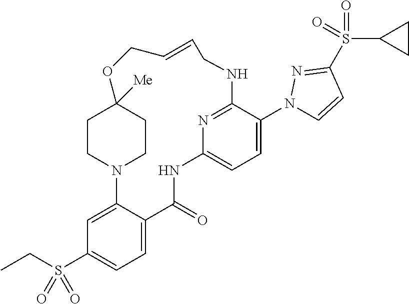 [SMILES: CCS(=O)(=O)c1ccc2c(c1)N1CCC(C)(CC1)OC/C=C/CNc1nc(ccc1-n1ccc(S(=O)(=O)C3CC3)n1)NC2=O] | 641.3 | 641.2199 |
| 378 | 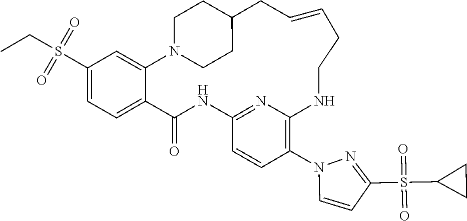 [SMILES: CCS(=O)(=O)c1ccc2c(c1)N1CCC(C/C=C/CCNc3nc(ccc3-n3ccc(S(=O)(=O)C4CC4)n3)NC2=O)CC1] | 625.3 | 625.225 |
| 379 | 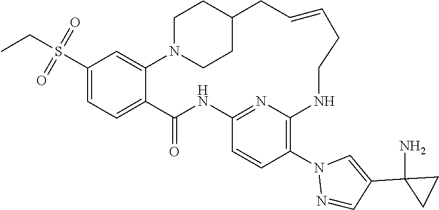 [SMILES: CCS(=O)(=O)c1ccc2c(c1)N1CCC(C/C=C/CCNc3nc(ccc3-n3cc(C4(N)CC4)cn3)NC2=O)CC1] | 576.4 | 576.2725 |
| 380 | 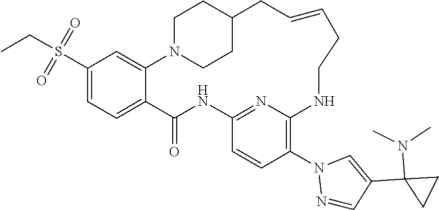 [SMILES: CCS(=O)(=O)c1ccc2c(c1)N1CCC(C/C=C/CCNc3nc(ccc3-n3cc(C4(N(C)C)CC4)cn3)NC2=O)CC1] | 604.4 | 604.3039 |
| 381 | 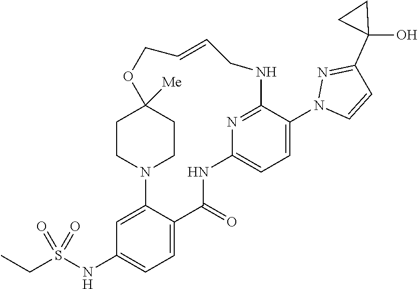 [SMILES: CCS(=O)(=O)Nc1ccc2c(c1)N1CCC(C)(CC1)OC/C=C/CNc1nc(ccc1-n1ccc(C3(O)CC3)n1)NC2=O] | 608.4 | 608.2619 |
| 382 | 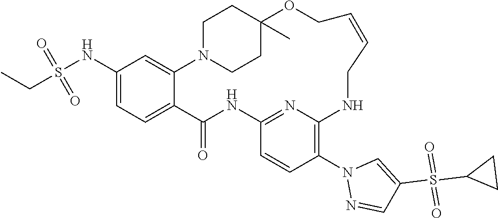 [SMILES: CCS(=O)(=O)Nc1ccc2c(c1)N1CCC(C)(CC1)OC/C=C\CNc1nc(ccc1-n1cc(S(=O)(=O)C3CC3)cn1)NC2=O] | 656.3 | 656.2302 |
| 383 | 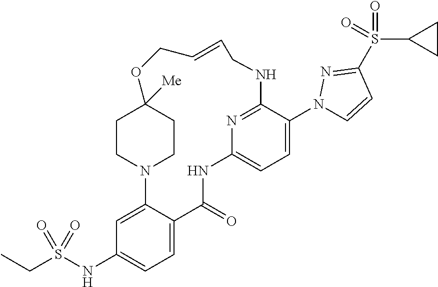 [SMILES: CCS(=O)(=O)Nc1ccc2c(c1)N1CCC(C)(CC1)OC/C=C/CNc1nc(ccc1-n1ccc(S(=O)(=O)C3CC3)n1)NC2=O] | 656.3 | 656.2302 |
| 384 | 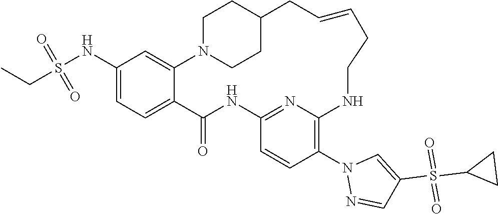 [SMILES: CCS(=O)(=O)Nc1ccc2c(c1)N1CCC(C/C=C/CCNc3nc(ccc3-n3cc(S(=O)(=O)C4CC4)cn3)NC2=O)CC1] | 640.3 | 640.2361 |
| 385 | 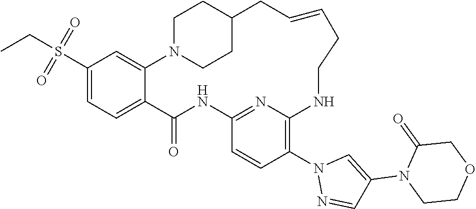 [SMILES: CCS(=O)(=O)c1ccc2c(c1)N1CCC(C/C=C/CCNc3nc(ccc3-n3cc(N4CCOCC4=O)cn3)NC2=O)CC1] | 620.3 | 620.2632 |
| 386 | 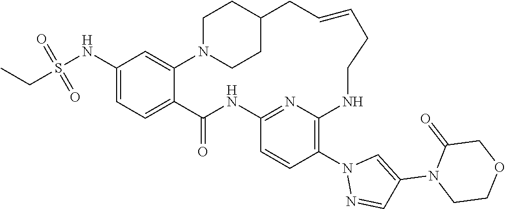 [SMILES: CCS(=O)(=O)Nc1ccc2c(c1)N1CCC(C/C=C/CCNc3nc(ccc3-n3cc(N4CCOCC4=O)cn3)NC2=O)CC1] | 635.3 | 635.2739 |
| 387 | 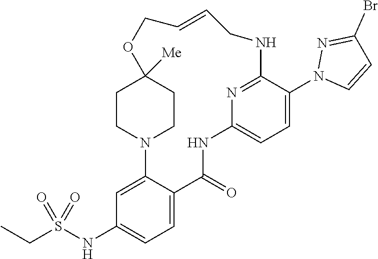 [SMILES: CCS(=O)(=O)Nc1ccc2c(c1)N1CCC(C)(CC1)OC/C=C/CNc1nc(ccc1-n1ccc(Br)n1)NC2=O] | | 630.1482 |
| 388 | 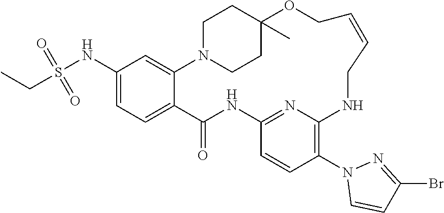 [SMILES: CCS(=O)(=O)Nc1ccc2c(c1)N1CCC(C)(CC1)OC/C=C\CNc1nc(ccc1-n1ccc(Br)n1)NC2=O] | | 630.1481 |
| 389 | 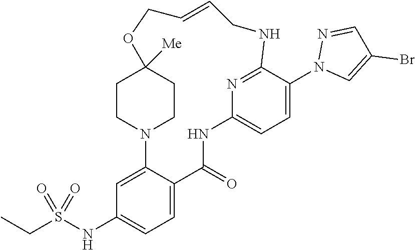 [SMILES: CCS(=O)(=O)Nc1ccc2c(c1)N1CCC(C)(CC1)OC/C=C/CNc1nc(ccc1-n1cc(Br)cn1)NC2=O] | | 630.1488 |
| 390 | 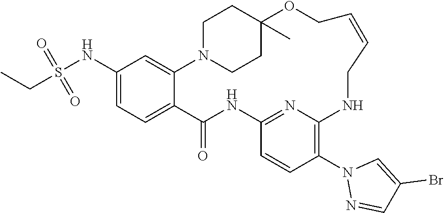 [SMILES: CCS(=O)(=O)Nc1ccc2c(c1)N1CCC(C)(CC1)OC/C=C\CNc1nc(ccc1-n1cc(Br)cn1)NC2=O] | | 630.1476 |
| 391 | 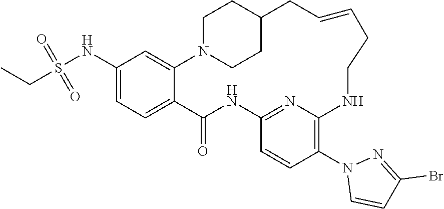 [SMILES: CCS(=O)(=O)Nc1ccc2c(c1)N1CCC(C/C=C/CCNc3nc(ccc3-n3ccc(Br)n3)NC2=O)CC1] | | 614.1536 |
| 392 | 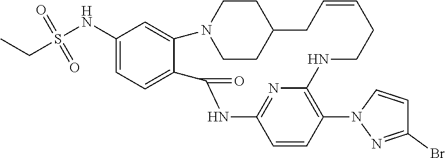 [SMILES: CCS(=O)(=O)Nc1ccc2c(c1)N1CCC(C/C=C\CCNc3nc(ccc3-n3ccc(Br)n3)NC2=O)CC1] | | 614.1534 |
| 393 | 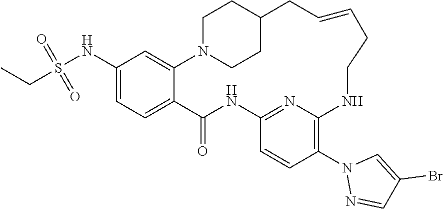 [SMILES: CCS(=O)(=O)Nc1ccc2c(c1)N1CCC(C/C=C/CCNc3nc(ccc3-n3cc(Br)cn3)NC2=O)CC1] | | 614.1545 |
| 394 | 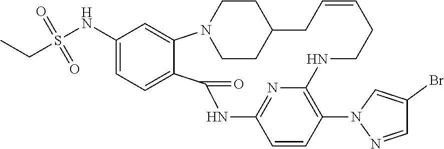 [SMILES: CCS(=O)(=O)Nc1ccc2c(c1)N1CCC(C/C=C\CCNc3nc(ccc3-n3cc(Br)cn3)NC2=O)CC1] | | 614.153 |
| 395 | 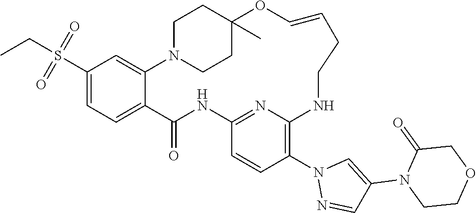 [SMILES: CCS(=O)(=O)c1ccc2c(c1)N1CCC(C)(CC1)O/C=C/CCNc1nc(ccc1-n1cc(N3CCOCC3=O)cn1)NC2=O] | 636.3 | 636.2588 |
| 396 | 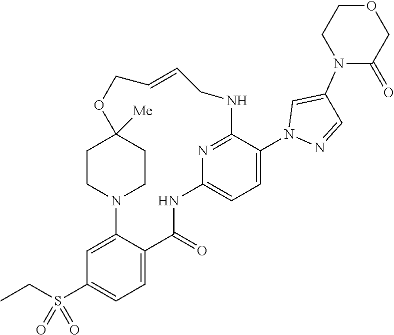 [SMILES: CCS(=O)(=O)c1ccc2c(c1)N1CCC(C)(CC1)OC/C=C/CNc1nc(ccc1-n1cc(N3CCOCC3=O)cn1)NC2=O] | 636.4 | 636.2589 |
| 397 | 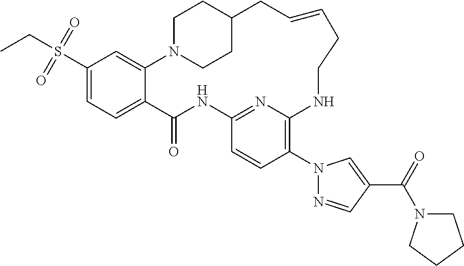 [SMILES: CCS(=O)(=O)c1ccc2c(c1)N1CCC(C/C=C/CCNc3nc(ccc3-n3cc(C(=O)N4CCCC4)cn3)NC2=O)CC1] | 618.4 | 618.2856 |
| 398 | 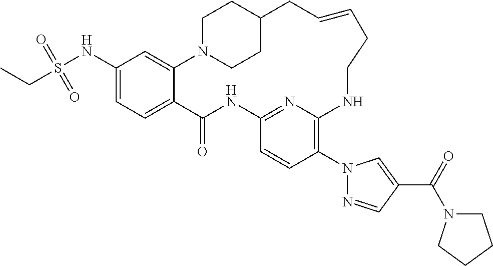 [SMILES: CCS(=O)(=O)Nc1ccc2c(c1)N1CCC(C/C=C/CCNc3nc(ccc3-n3cc(C(=O)N4CCCC4)cn3)NC2=O)CC1] | 633.4 | 633.2956 |
| 399 | 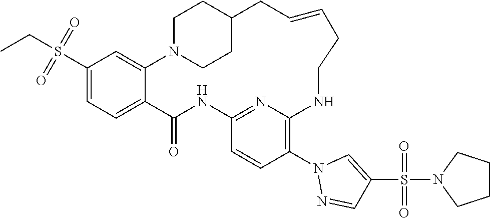 [SMILES: CCS(=O)(=O)c1ccc2c(c1)N1CCC(C/C=C/CCNc3nc(ccc3-n3cc(S(=O)(=O)N4CCCC4)cn3)NC2=O)CC1] | 654.3 | 654.2504 |
| 400 | 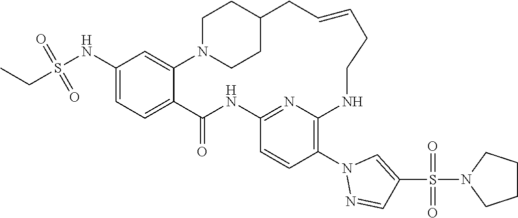 [SMILES: CCS(=O)(=O)Nc1ccc2c(c1)N1CCC(C/C=C/CCNc3nc(ccc3-n3cc(S(=O)(=O)N4CCCC4)cn3)NC2=O)CC1] | 669.3 | 669.2626 |
| 401 | 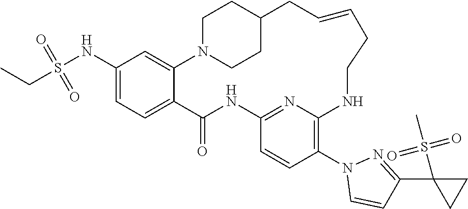 [SMILES: CCS(=O)(=O)Nc1ccc2c(c1)N1CCC(C/C=C/CCNc3nc(ccc3-n3ccc(C4(S(C)(=O)=O)CC4)n3)NC2=O)CC1] | 654.3 | 654.251 |
| 402 | 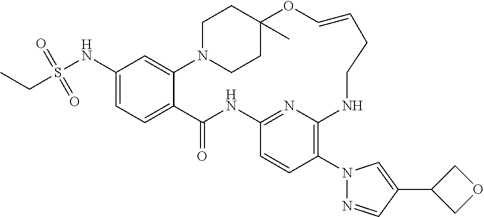 [SMILES: CCS(=O)(=O)Nc1ccc2c(c1)N1CCC(C)(CC1)O/C=C/CCNc1nc(ccc1-n1cc(C3COC3)cn1)NC2=O] | 608.4 | 608.2638 |
| 403 | 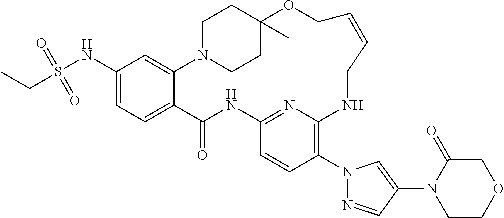 [SMILES: CCS(=O)(=O)Nc1ccc2c(c1)N1CCC(C)(CC1)OC/C=C\CNc1nc(ccc1-n1cc(N3CCOCC3=O)cn1)NC2=O] | 651.3 | 651.2691 |
| 404 | 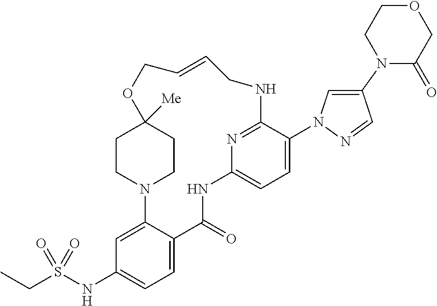 [SMILES: CCS(=O)(=O)Nc1ccc2c(c1)N1CCC(C)(CC1)OC/C=C/CNc1nc(ccc1-n1cc(N3CCOCC3=O)cn1)NC2=O] | 651.4 | 651.2697 |
| 405 | 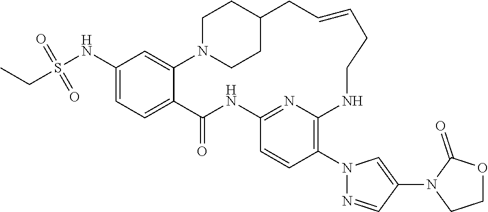 [SMILES: CCS(=O)(=O)Nc1ccc2c(c1)N1CCC(C/C=C/CCNc3nc(ccc3-n3cc(N4CCOC4=O)cn3)NC2=O)CC1] | 621.3 | 621.2588 |
| 406 | 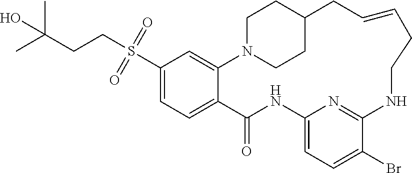 [SMILES: CC(C)(O)CCS(=O)(=O)c1ccc2c(c1)N1CCC(C/C=C/CCNc3nc(ccc3Br)NC2=O)CC1] | 593 | |
| 407 | 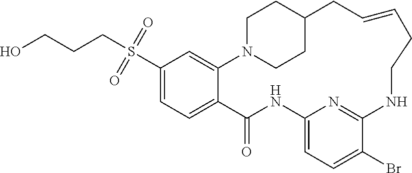 [SMILES: O=C1Nc2ccc(Br)c(n2)NCC/C=C/CC2CCN(CC2)c2cc(S(=O)(=O)CCCO)ccc21] | 565.1 | 563.1299 |
| 408 | 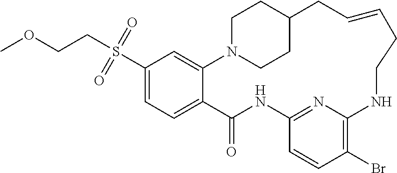 [SMILES: COCCS(=O)(=O)c1ccc2c(c1)N1CCC(C/C=C/CCNc3nc(ccc3Br)NC2=O)CC1] | 565.1 | 563.1306 |
| 409 | 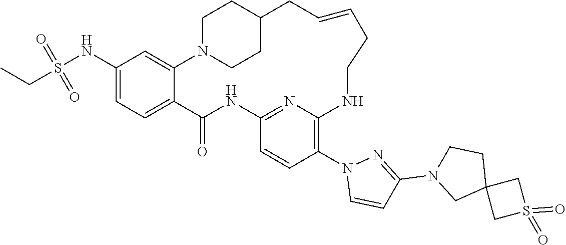 [SMILES: CCS(=O)(=O)Nc1ccc2c(c1)N1CCC(C/C=C/CCNc3nc(ccc3-n3ccc(N4CCC5(C4)CS(=O)(=O)C5)n3)NC2=O)CC1] | 695.2 | 695.2782 |
| 410 | 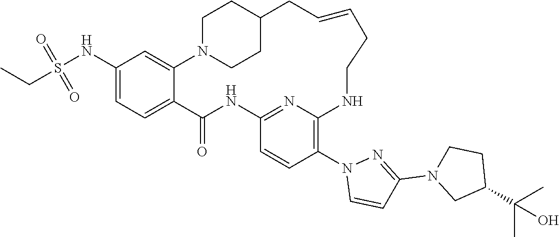 [SMILES: CCS(=O)(=O)Nc1ccc2c(c1)N1CCC(C/C=C/CCNc3nc(ccc3-n3ccc(N4CC[C@H](C(C)(C)O)C4)n3)NC2=O)CC1] | 663.3 | 663.3401 |
| 411 | 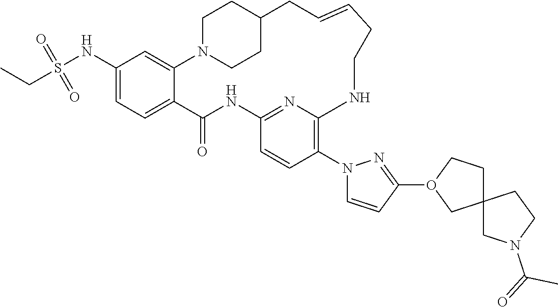 | 702.3 | 702.3517 |
| 412 | 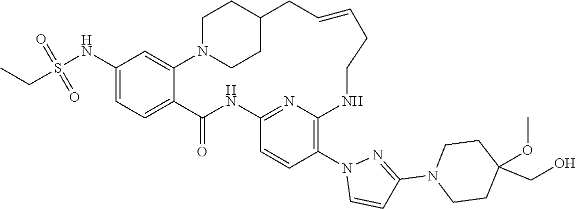 [SMILES: CCS(=O)(=O)Nc1ccc2c(c1)N1CCC(C/C=C/CCNc3nc(ccc3-n3ccc(N4CCC(CO)(OC)CC4)n3)NC2=O)CC1] | 679.3 | 679.3364 |
| 413 | 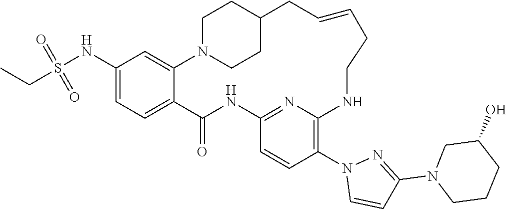 [SMILES: CCS(=O)(=O)Nc1ccc2c(c1)N1CCC(C/C=C/CCNc3nc(ccc3-n3ccc(N4CCC[C@@H](O)C4)n3)NC2=O)CC1] | 635.2 | 635.3104 |
| 414 | 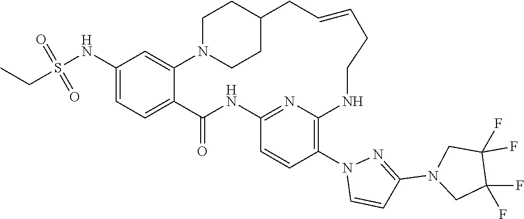 [SMILES: CCS(=O)(=O)Nc1ccc2c(c1)N1CCC(C/C=C/CCNc3nc(ccc3-n3ccc(N4CC(F)(F)C(F)(F)C4)n3)NC2=O)CC1] | 677.2 | 677.2622 |
| 415 | 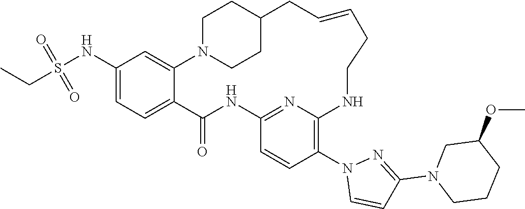 [SMILES: CCS(=O)(=O)Nc1ccc2c(c1)N1CCC(C/C=C/CCNc3nc(ccc3-n3ccc(N4CCC[C@H](OC)C4)n3)NC2=O)CC1] | 649.2 | 649.3268 |
| 416 | 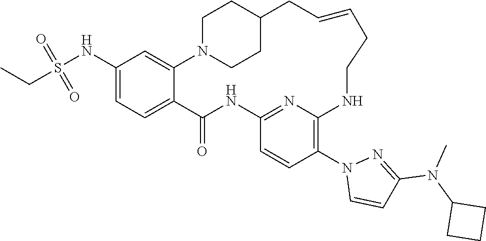 [SMILES: CCS(=O)(=O)Nc1ccc2c(c1)N1CCC(C/C=C/CCNc3nc(ccc3-n3ccc(N(C)C4CCC4)n3)NC2=O)CC1] | 619.2 | 619.3149 |
| 417 | 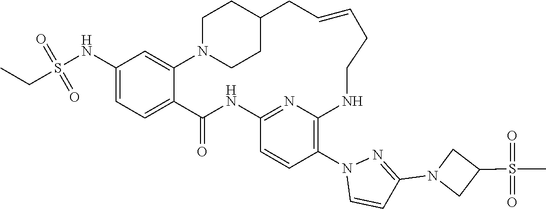 [SMILES: CCS(=O)(=O)Nc1ccc2c(c1)N1CCC(C/C=C/CCNc3nc(ccc3-n3ccc(N4CC(S(C)(=O)=O)C4)n3)NC2=O)CC1] | 669.2 | 669.2626 |
| 418 | 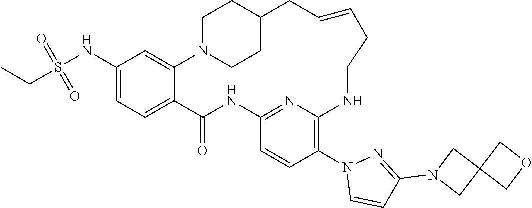 [SMILES: CCS(=O)(=O)Nc1ccc2c(c1)N1CCC(C/C=C/CCNc3nc(ccc3-n3ccc(N4CC5(COC5)C4)n3)NC2=O)CC1] | 633.2 | 633.2954 |
| 419 | 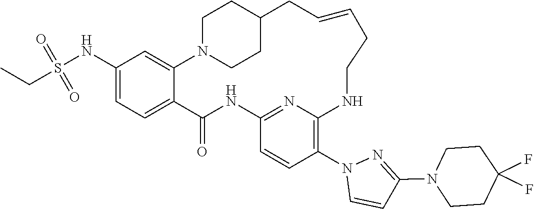 [SMILES: CCS(=O)(=O)Nc1ccc2c(c1)N1CCC(C/C=C/CCNc3nc(ccc3-n3ccc(N4CCC(F)(F)CC4)n3)NC2=O)CC1] | 655.2 | 655.2969 |
| 420 | 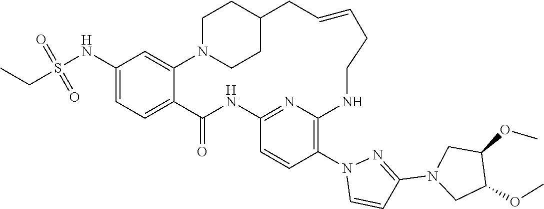 [SMILES: CCS(=O)(=O)Nc1ccc2c(c1)N1CCC(C/C=C/CCNc3nc(ccc3-n3ccc(N4C[C@@H](OC)[C@H](OC)C4)n3)NC2=O)CC1] | 665.2 | 665.3212 |
| 421 | 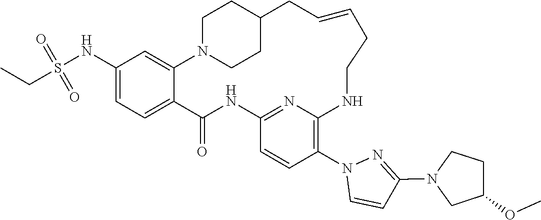 [SMILES: CCS(=O)(=O)Nc1ccc2c(c1)N1CCC(C/C=C/CCNc3nc(ccc3-n3ccc(N4CC[C@H](OC)C4)n3)NC2=O)CC1] | 635.2 | 635.3102 |
| 422 | 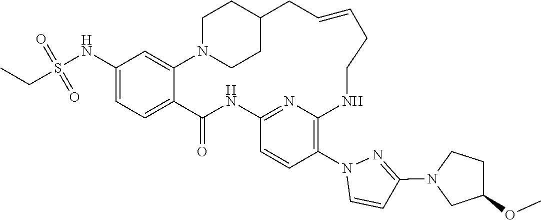 [SMILES: CCS(=O)(=O)Nc1ccc2c(c1)N1CCC(C/C=C/CCNc3nc(ccc3-n3ccc(N4CC[C@@H](OC)C4)n3)NC2=O)CC1] | 635.3 | 635.3098 |
| 423 | 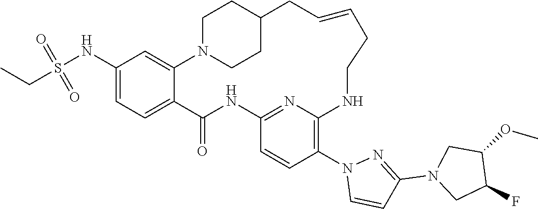 [SMILES: CCS(=O)(=O)Nc1ccc2c(c1)N1CCC(C/C=C/CCNc3nc(ccc3-n3ccc(N4C[C@H](OC)[C@@H](F)C4)n3)NC2=O)CC1] | 653.2 | 653.3007 |
| 424 | 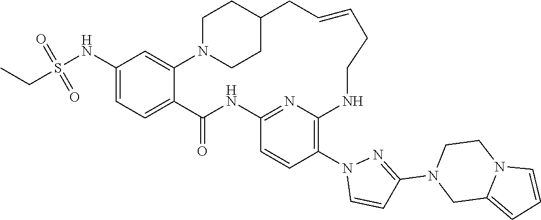 [SMILES: CCS(=O)(=O)Nc1ccc2c(c1)N1CCC(C/C=C/CCNc3nc(ccc3-n3ccc(N4CCn5cccc5C4)n3)NC2=O)CC1] | 656.2 | 656.3105 |
| 425 | 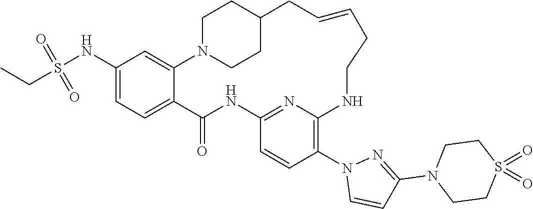 [SMILES: CCS(=O)(=O)Nc1ccc2c(c1)N1CCC(C/C=C/CCNc3nc(ccc3-n3ccc(N4CCS(=O)(=O)CC4)n3)NC2=O)CC1] | 669.2 | 669.2623 |
| 426 | 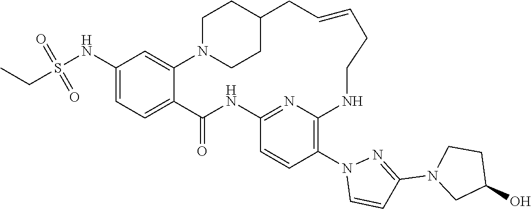 [SMILES: CCS(=O)(=O)Nc1ccc2c(c1)N1CCC(C/C=C/CCNc3nc(ccc3-n3ccc(N4CC[C@@H](O)C4)n3)NC2=O)CC1] | 621.2 | 621.2947 |
| 427 | 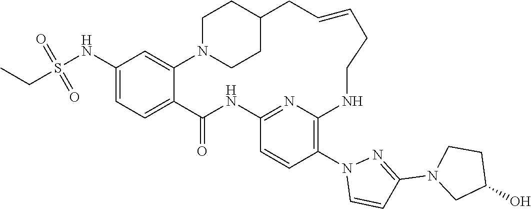 [SMILES: CCS(=O)(=O)Nc1ccc2c(c1)N1CCC(C/C=C/CCNc3nc(ccc3-n3ccc(N4CC[C@H](O)C4)n3)NC2=O)CC1] | 621.2 | 621.2937 |
| 428 | 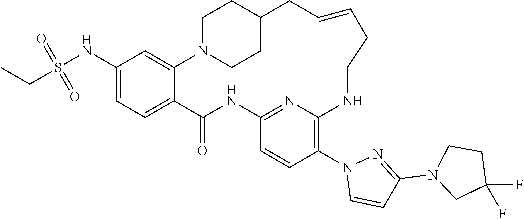 [SMILES: CCS(=O)(=O)Nc1ccc2c(c1)N1CCC(C/C=C/CCNc3nc(ccc3-n3ccc(N4CCC(F)(F)C4)n3)NC2=O)CC1] | 641.2 | 641.2815 |
| 429 | 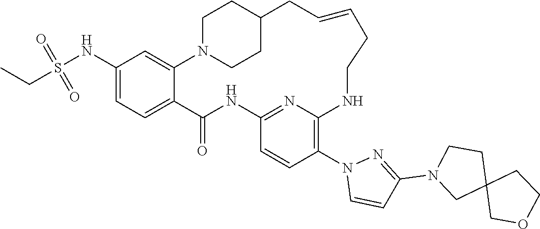 [SMILES: CCS(=O)(=O)Nc1ccc2c(c1)N1CCC(C/C=C/CCNc3nc(ccc3-n3ccc(N4CCC5(CCOC5)C4)n3)NC2=O)CC1] | 661.2 | 661.325 |
| 430 | 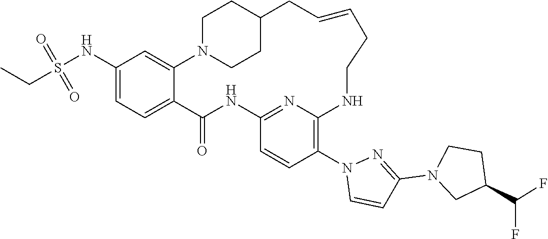 [SMILES: CCS(=O)(=O)Nc1ccc2c(c1)N1CCC(C/C=C/CCNc3nc(ccc3-n3ccc(N4CC[C@@H](C(F)F)C4)n3)NC2=O)CC1] | 655.2 | 655.2978 |
| 431 | 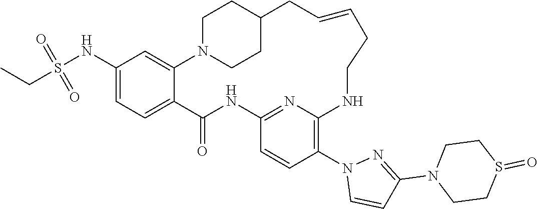 [SMILES: CCS(=O)(=O)Nc1ccc2c(c1)N1CCC(C/C=C/CCNc3nc(ccc3-n3ccc(N4CCS(=O)CC4)n3)NC2=O)CC1] | 653.1 | 653.2671 |
| 432 | 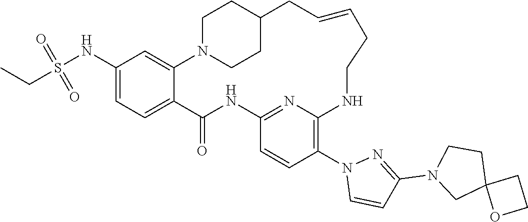 [SMILES: CCS(=O)(=O)Nc1ccc2c(c1)N1CCC(C/C=C/CCNc3nc(ccc3-n3ccc(N4CCC5(CCO5)C4)n3)NC2=O)CC1] | 647.2 | 647.311 |
| 433 | 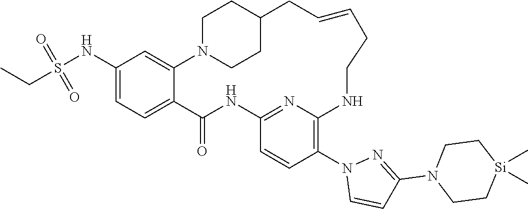 [SMILES: CCS(=O)(=O)Nc1ccc2c(c1)N1CCC(C/C=C/CCNc3nc(ccc3-n3ccc(N4CC[Si](C)(C)CC4)n3)NC2=O)CC1] | 663.3 | 663.3243 |
| 434 | 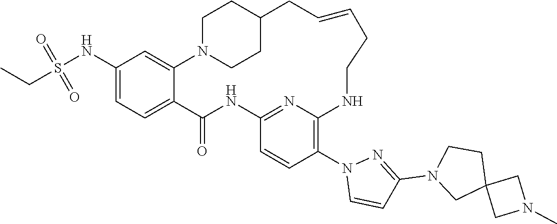 [SMILES: CCS(=O)(=O)Nc1ccc2c(c1)N1CCC(C/C=C/CCNc3nc(ccc3-n3ccc(N4CCC5(CN(C)C5)C4)n3)NC2=O)CC1] | 660.2 | 660.3427 |
| 435 | 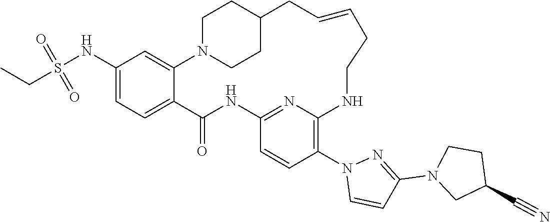 [SMILES: CCS(=O)(=O)Nc1ccc2c(c1)N1CCC(C/C=C/CCNc3nc(ccc3-n3ccc(N4CC[C@@H](C#N)C4)n3)NC2=O)CC1] | 630.2 | 630.2961 |
| 436 | 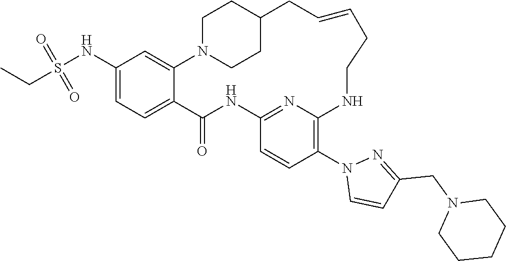 [SMILES: CCS(=O)(=O)Nc1ccc2c(c1)N1CCC(C/C=C/CCNc3nc(ccc3-n3ccc(CN4CCCCC4)n3)NC2=O)CC1] | 633.2 | 633.3308 |
| 437 | 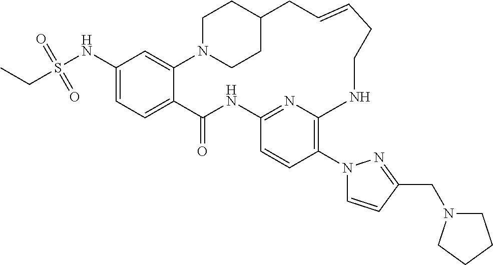 [SMILES: CCS(=O)(=O)Nc1ccc2c(c1)N1CCC(C/C=C/CCNc3nc(ccc3-n3ccc(CN4CCCC4)n3)NC2=O)CC1] | 619.2 | 619.3145 |
| 438 | 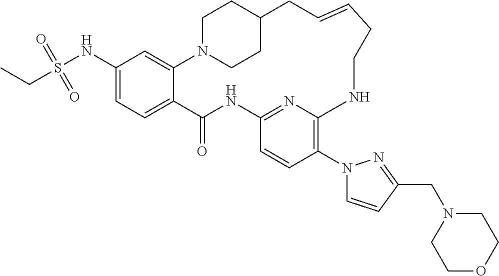 [SMILES: CCS(=O)(=O)Nc1ccc2c(c1)N1CCC(C/C=C/CCNc3nc(ccc3-n3ccc(CN4CCOCC4)n3)NC2=O)CC1] | 635.2 | 635.3101 |
| 439 | 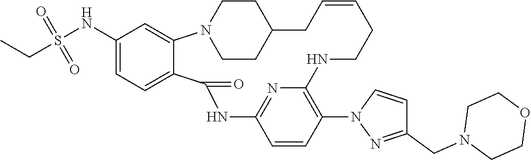 [SMILES: CCS(=O)(=O)Nc1ccc2c(c1)N1CCC(C/C=C\CCNc3nc(ccc3-n3ccc(CN4CCOCC4)n3)NC2=O)CC1] | 635.1 | 635.3091 |
| 440 | 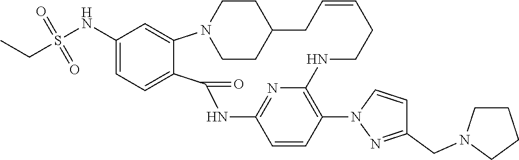 [SMILES: CCS(=O)(=O)Nc1ccc2c(c1)N1CCC(C/C=C\CCNc3nc(ccc3-n3ccc(CN4CCCC4)n3)NC2=O)CC1] | 619.1 | 619.3154 |
| 441 | 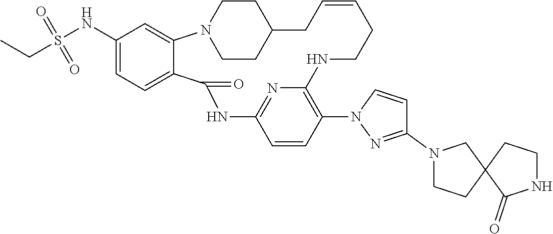 [SMILES: CCS(=O)(=O)Nc1ccc2c(c1)N1CCC(C/C=C\CCNc3nc(ccc3-n3ccc(N4CCC5(CCNC5=O)C4)n3)NC2=O)CC1] | 674.3 | 674.3194 |
| 442 | 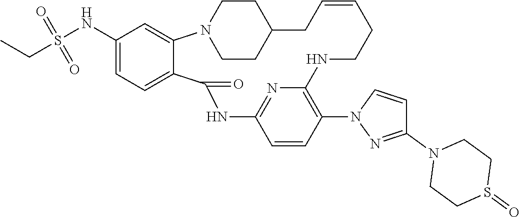 [SMILES: CCS(=O)(=O)Nc1ccc2c(c1)N1CCC(C/C=C\CCNc3nc(ccc3-n3ccc(N4CCS(=O)CC4)n3)NC2=O)CC1] | 653.1 | 653.2652 |
| 443 | 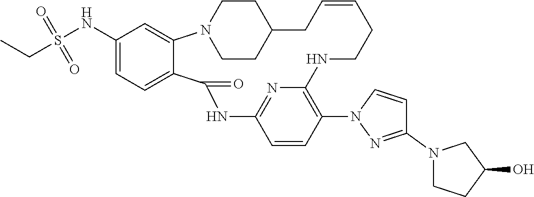 [SMILES: CCS(=O)(=O)Nc1ccc2c(c1)N1CCC(C/C=C\CCNc3nc(ccc3-n3ccc(N4CC[C@H](O)C4)n3)NC2=O)CC1] | 621.3 | 621.2931 |
| 444 | 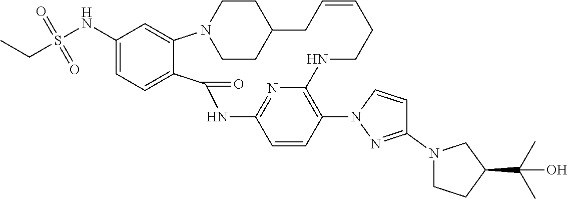 [SMILES: CCS(=O)(=O)Nc1ccc2c(c1)N1CCC(C/C=C\CCNc3nc(ccc3-n3ccc(N4CC[C@H](C(C)(C)O)C4)n3)NC2=O)CC1] | 663.3 | 663.3412 |
| 445 | 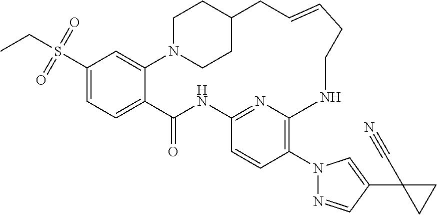 [SMILES: CCS(=O)(=O)c1ccc2c(c1)N1CCC(C/C=C/CCNc3nc(ccc3-n3cc(C4(C#N)CC4)cn3)NC2=O)CC1] | 586.3 | 586.2584 |
| 446 | 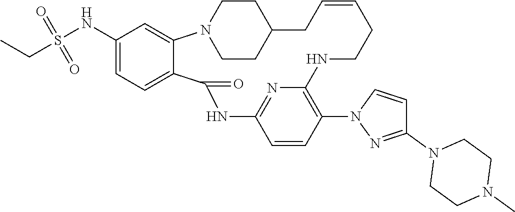 [SMILES: CCS(=O)(=O)Nc1ccc2c(c1)N1CCC(C/C=C\CCNc3nc(ccc3-n3ccc(N4CCN(C)CC4)n3)NC2=O)CC1] | 634.2 | 634.3277 |
| 447 | 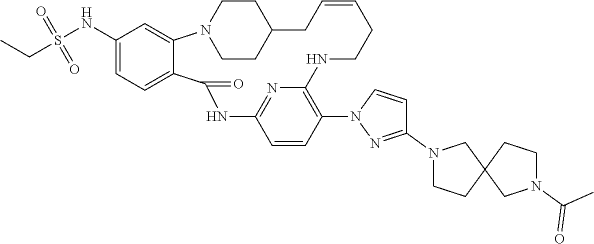 [SMILES: CCS(=O)(=O)Nc1ccc2c(c1)N1CCC(C/C=C\CCNc3nc(ccc3-n3ccc(N4CCC5(CCN(C(C)=O)C5)C4)n3)NC2=O)CC1] | 702.3 | 702.3533 |
| 448 | 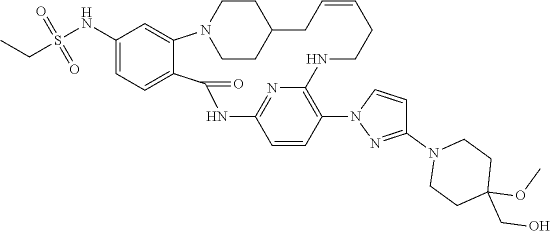 [SMILES: CCS(=O)(=O)Nc1ccc2c(c1)N1CCC(C/C=C\CCNc3nc(ccc3-n3ccc(N4CCC(CO)(OC)CC4)n3)NC2=O)CC1] | 679.3 | 679.3358 |
| 449 | 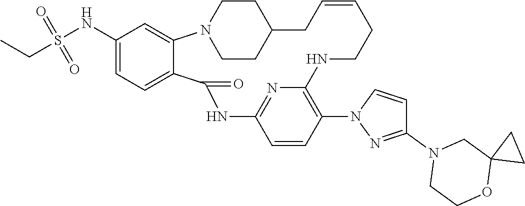 [SMILES: CCS(=O)(=O)Nc1ccc2c(c1)N1CCC(C/C=C\CCNc3nc(ccc3-n3ccc(N4CCOC5(CC5)C4)n3)NC2=O)CC1] | 647.2 | 647.3094 |
| 450 | 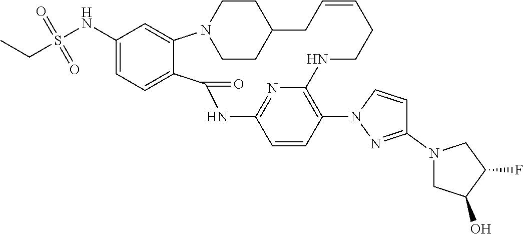 [SMILES: CCS(=O)(=O)Nc1ccc2c(c1)N1CCC(C/C=C\CCNc3nc(ccc3-n3ccc(N4C[C@H](O)[C@@H](F)C4)n3)NC2=O)CC1] | 639.1 | 639.2855 |
| 451 | 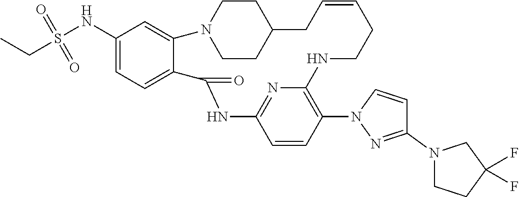 [SMILES: CCS(=O)(=O)Nc1ccc2c(c1)N1CCC(C/C=C\CCNc3nc(ccc3-n3ccc(N4CCC(F)(F)C4)n3)NC2=O)CC1] | 641.1 | 641.2806 |
| 452 | 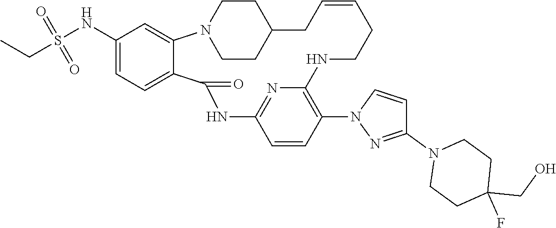 [SMILES: CCS(=O)(=O)Nc1ccc2c(c1)N1CCC(C/C=C\CCNc3nc(ccc3-n3ccc(N4CCC(F)(CO)CC4)n3)NC2=O)CC1] | 667.3 | 667.3172 |
| 453 | 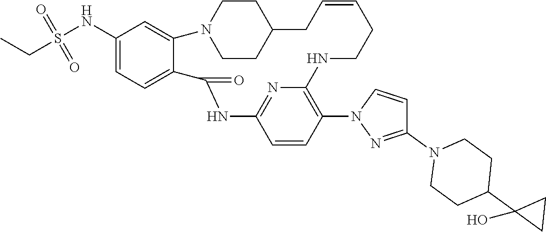 [SMILES: CCS(=O)(=O)Nc1ccc2c(c1)N1CCC(C/C=C\CCNc3nc(ccc3-n3ccc(N4CCC(C5(O)CC5)CC4)n3)NC2=O)CC1] | 675.3 | 675.3407 |
| 454 | 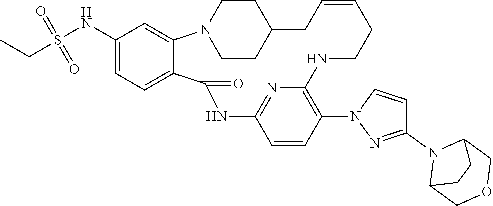 [SMILES: CCS(=O)(=O)Nc1ccc2c(c1)N1CCC(C/C=C\CCNc3nc(ccc3-n3ccc(N4C5CCC4COC5)n3)NC2=O)CC1] | 647.2 | 647.31 |
| 455 | 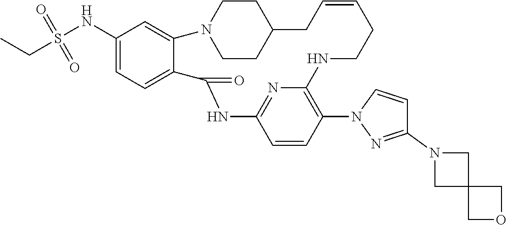 [SMILES: CCS(=O)(=O)Nc1ccc2c(c1)N1CCC(C/C=C\CCNc3nc(ccc3-n3ccc(N4CC5(COC5)C4)n3)NC2=O)CC1] | 633.2 | 633.2951 |
| 456 | 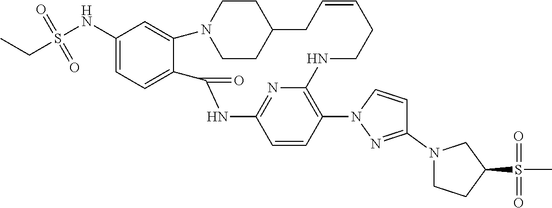 [SMILES: CCS(=O)(=O)Nc1ccc2c(c1)N1CCC(C/C=C\CCNc3nc(ccc3-n3ccc(N4CC[C@H](S(C)(=O)=O)C4)n3)NC2=O)CC1] | 683.2 | 683.2774 |
| 457 | 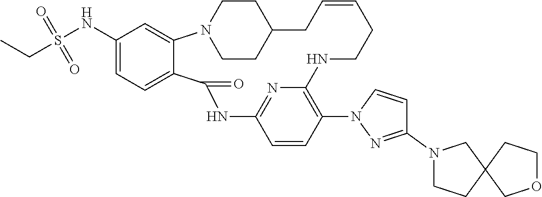 [SMILES: CCS(=O)(=O)Nc1ccc2c(c1)N1CCC(C/C=C\CCNc3nc(ccc3-n3ccc(N4CCC5(CCOC5)C4)n3)NC2=O)CC1] | 661.2 | 661.325 |
| 458 | 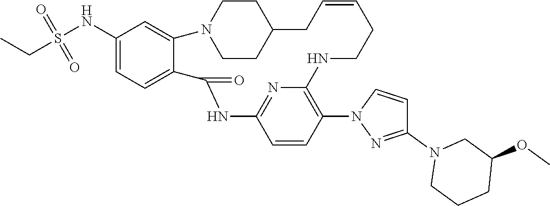 [SMILES: CCS(=O)(=O)Nc1ccc2c(c1)N1CCC(C/C=C\CCNc3nc(ccc3-n3ccc(N4CCC[C@H](OC)C4)n3)NC2=O)CC1] | 649.1 | 649.3257 |
| 459 | 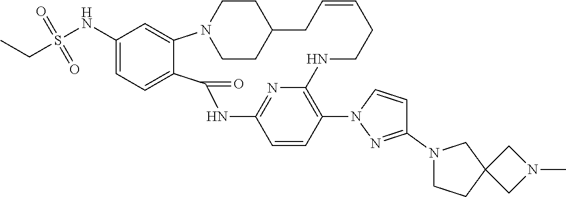 [SMILES: CCS(=O)(=O)Nc1ccc2c(c1)N1CCC(C/C=C\CCNc3nc(ccc3-n3ccc(N4CCC5(CN(C)C5)C4)n3)NC2=O)CC1] | 660.3 | 660.3437 |
| 460 | 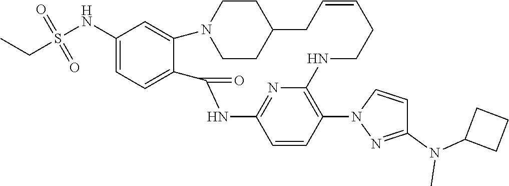 [SMILES: CCS(=O)(=O)Nc1ccc2c(c1)N1CCC(C/C=C\CCNc3nc(ccc3-n3ccc(N(C)C4CCC4)n3)NC2=O)CC1] | 619.2 | 619.3151 |
| 461 | 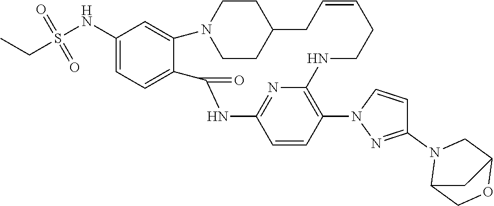 [SMILES: CCS(=O)(=O)Nc1ccc2c(c1)N1CCC(C/C=C\CCNc3nc(ccc3-n3ccc(N4CC5CC4CO5)n3)NC2=O)CC1] | 633.2 | 633.2951 |
| 462 | 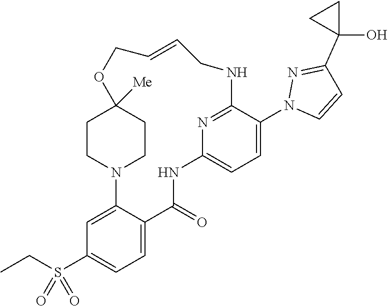 [SMILES: CCS(=O)(=O)c1ccc2c(c1)N1CCC(C)(CC1)OC/C=C/CNc1nc(ccc1-n1ccc(C3(O)CC3)n1)NC2=O] | 593.3 | 593.2531 |
| 463 | 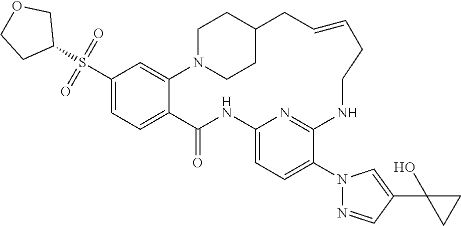 [SMILES: O=C1Nc2ccc(-n3cc(C4(O)CC4)cn3)c(n2)NCC/C=C/CC2CCN(CC2)c2cc(S(=O)(=O)[C@@H]3CCOC3)ccc21] | 619.3 | 619.2681 |
| 464 | 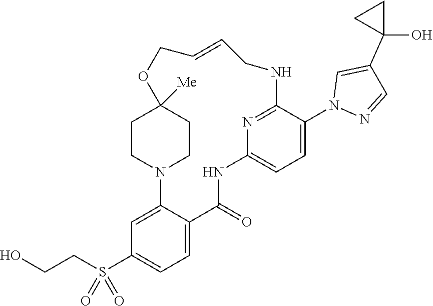 [SMILES: CC12CCN(CC1)c1cc(S(=O)(=O)CCO)ccc1C(=O)Nc1ccc(-n3cc(C4(O)CC4)cn3)c(n1)NC/C=C/CO2] | 609.3 | 609.2471 |
| 465 | 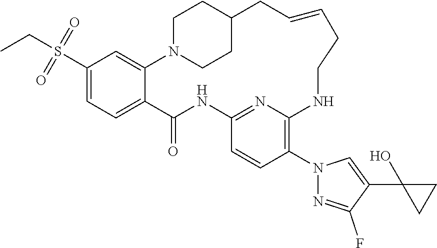 [SMILES: CCS(=O)(=O)c1ccc2c(c1)N1CCC(C/C=C/CCNc3nc(ccc3-n3cc(C4(O)CC4)c(F)n3)NC2=O)CC1] | 595.3 | 595.2473 |
| 466 | 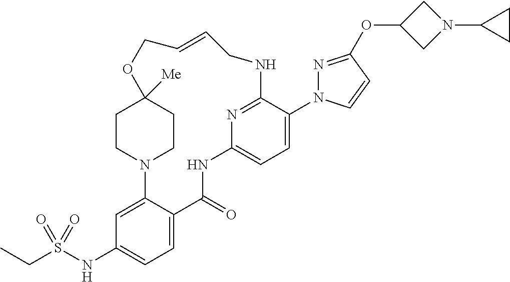 [SMILES: CCS(=O)(=O)Nc1ccc2c(c1)N1CCC(C)(CC1)OC/C=C/CNc1nc(ccc1-n1ccc(OC3CN(C4CC4)C3)n1)NC2=O] | 663.2 | 663.3069 |
| 467 | 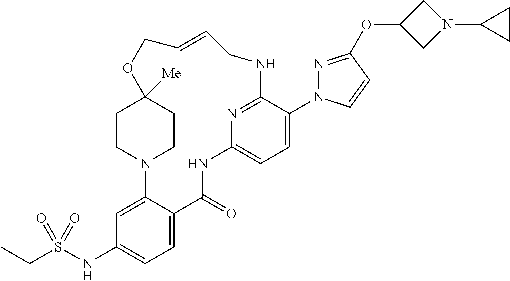 [SMILES: CCS(=O)(=O)Nc1ccc2c(c1)N1CCC(C)(CC1)OC/C=C/CNc1nc(ccc1-n1ccc(OC3CN(C4CC4)C3)n1)NC2=O] | 665.2 | 665.3227 |
| 468 | 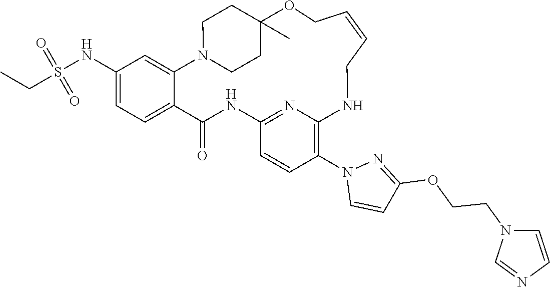 [SMILES: CCS(=O)(=O)Nc1ccc2c(c1)N1CCC(C)(CC1)OC/C=C\CNc1nc(ccc1-n1ccc(OCCn3ccnc3)n1)NC2=O] | 662.2 | 662.2856 |
| 469 | 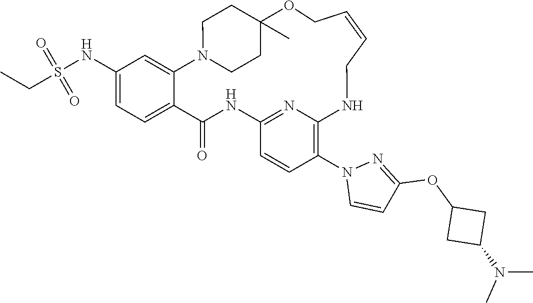 | 665.2 | 665.3215 |
| 470 | 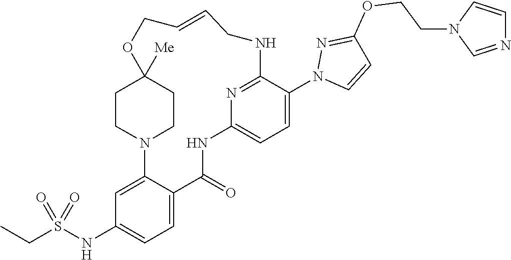 [SMILES: CCS(=O)(=O)Nc1ccc2c(c1)N1CCC(C)(CC1)OC/C=C/CNc1nc(ccc1-n1ccc(OCCn3ccnc3)n1)NC2=O] | 662.3 | 662.2862 |
| 471 | 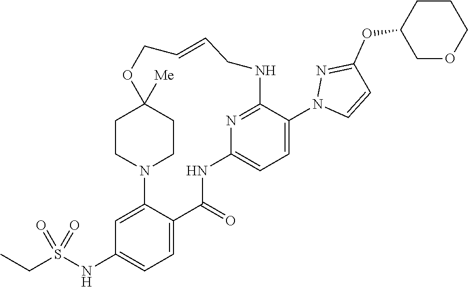 [SMILES: CCS(=O)(=O)Nc1ccc2c(c1)N1CCC(C)(CC1)OC/C=C/CNc1nc(ccc1-n1ccc(O[C@@H]3CCCOC3)n1)NC2=O] | 652.1 | 652.2904 |
| 472 | 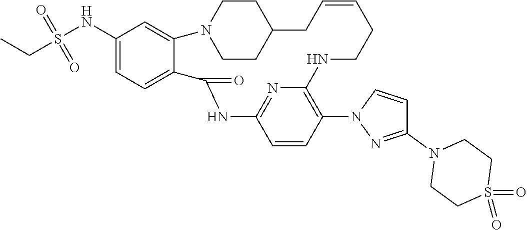 [SMILES: CCS(=O)(=O)Nc1ccc2c(c1)N1CCC(C/C=C\CCNc3nc(ccc3-n3ccc(N4CCS(=O)(=O)CC4)n3)NC2=O)CC1] | 668.9 | 669.2624 |
| 473 | 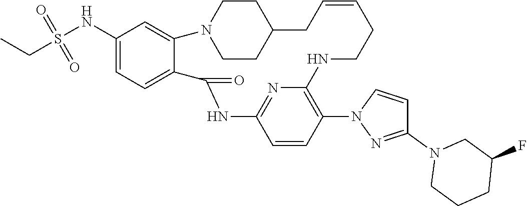 [SMILES: CCS(=O)(=O)Nc1ccc2c(c1)N1CCC(C/C=C\CCNc3nc(ccc3-n3ccc(N4CCC[C@H](F)C4)n3)NC2=O)CC1] | 637.2 | 637.3059 |
| 474 | 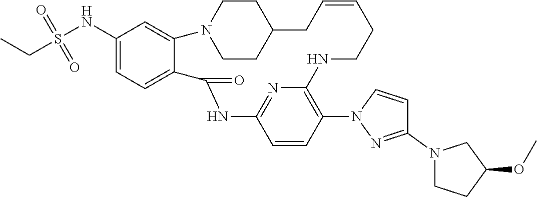 [SMILES: CCS(=O)(=O)Nc1ccc2c(c1)N1CCC(C/C=C\CCNc3nc(ccc3-n3ccc(N4CC[C@H](OC)C4)n3)NC2=O)CC1] | 635.2 | 635.311 |
| 475 | 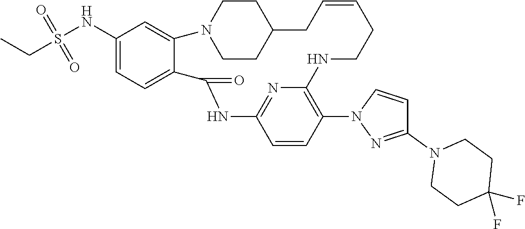 [SMILES: CCS(=O)(=O)Nc1ccc2c(c1)N1CCC(C/C=C\CCNc3nc(ccc3-n3ccc(N4CCC(F)(F)CC4)n3)NC2=O)CC1] | 655.2 | 655.2967 |
| 476 | 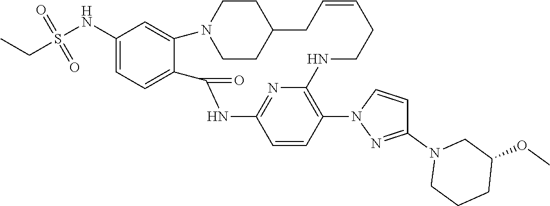 [SMILES: CCS(=O)(=O)Nc1ccc2c(c1)N1CCC(C/C=C\CCNc3nc(ccc3-n3ccc(N4CCC[C@@H](OC)C4)n3)NC2=O)CC1] | 649.2 | 649.3259 |
| 477 | 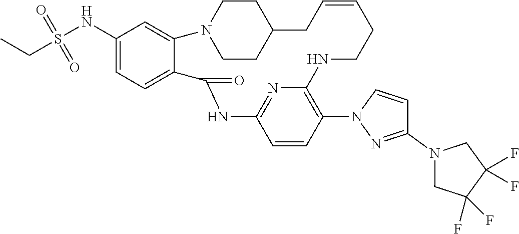 [SMILES: CCS(=O)(=O)Nc1ccc2c(c1)N1CCC(C/C=C\CCNc3nc(ccc3-n3ccc(N4CC(F)(F)C(F)(F)C4)n3)NC2=O)CC1] | 677.2 | 677.262 |
| 478 | 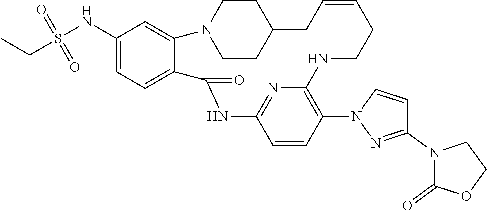 [SMILES: CCS(=O)(=O)Nc1ccc2c(c1)N1CCC(C/C=C\CCNc3nc(ccc3-n3ccc(N4CCOC4=O)n3)NC2=O)CC1] | 621.2 | 621.2587 |
| 479 | 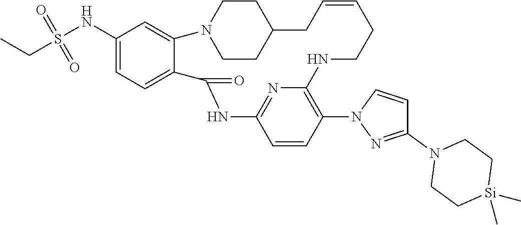 [SMILES: CCS(=O)(=O)Nc1ccc2c(c1)N1CCC(C/C=C\CCNc3nc(ccc3-n3ccc(N4CC[Si](C)(C)CC4)n3)NC2=O)CC1] | 663.2 | 663.3236 |
| 480 | 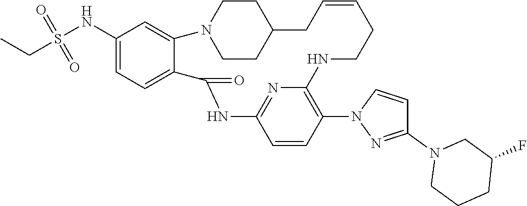 [SMILES: CCS(=O)(=O)Nc1ccc2c(c1)N1CCC(C/C=C\CCNc3nc(ccc3-n3ccc(N4CCC[C@@H](F)C4)n3)NC2=O)CC1] | 637.2 | 637.3059 |
| 481 | 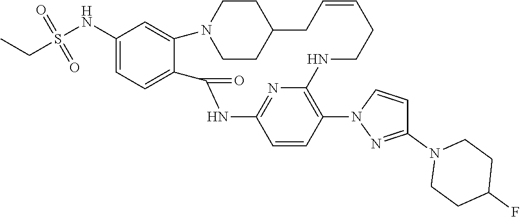 [SMILES: CCS(=O)(=O)Nc1ccc2c(c1)N1CCC(C/C=C\CCNc3nc(ccc3-n3ccc(N4CCC(F)CC4)n3)NC2=O)CC1] | 637.2 | 637.3064 |
| 482 | 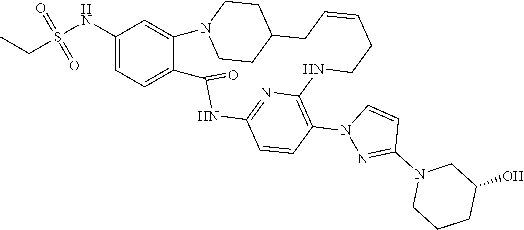 [SMILES: CCS(=O)(=O)Nc1ccc2c(c1)N1CCC(C/C=C\CCNc3nc(ccc3-n3ccc(N4CCC[C@@H](O)C4)n3)NC2=O)CC1] | 635.2 | 635.3109 |
| 483 | 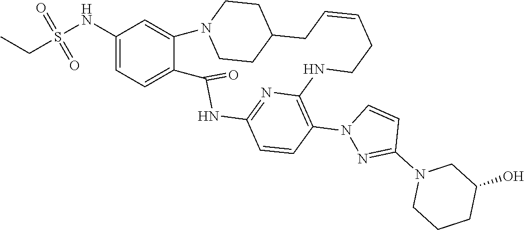 [SMILES: CCS(=O)(=O)Nc1ccc2c(c1)N1CCC(C/C=C\CCNc3nc(ccc3-n3ccc(N4CCC[C@@H](O)C4)n3)NC2=O)CC1] | 635.2 | 635.3111 |
| 484 | 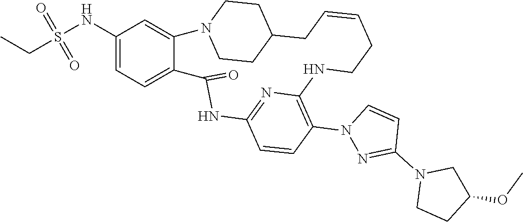 [SMILES: CCS(=O)(=O)Nc1ccc2c(c1)N1CCC(C/C=C\CCNc3nc(ccc3-n3ccc(N4CC[C@@H](OC)C4)n3)NC2=O)CC1] | 635.1 | 635.3102 |
| 485 | 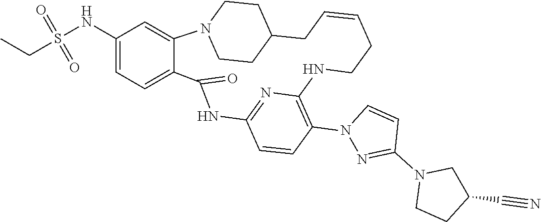 [SMILES: CCS(=O)(=O)Nc1ccc2c(c1)N1CCC(C/C=C\CCNc3nc(ccc3-n3ccc(N4CC[C@@H](C#N)C4)n3)NC2=O)CC1] | 630.2 | 630.2956 |
| 486 | 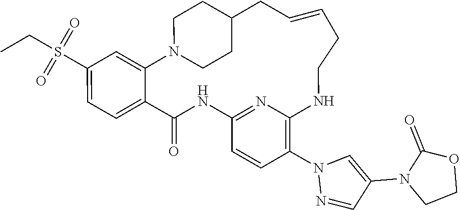 [SMILES: CCS(=O)(=O)c1ccc2c(c1)N1CCC(C/C=C/CCNc3nc(ccc3-n3cc(N4CCOC4=O)cn3)NC2=O)CC1] | 606.3 | 606.2479 |
| 487 | 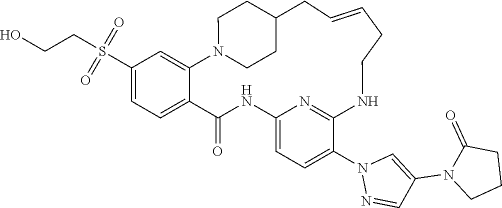 [SMILES: O=C1Nc2ccc(-n3cc(N4CCCC4=O)cn3)c(n2)NCC/C=C/CC2CCN(CC2)c2cc(S(=O)(=O)CCO)ccc21] | 620.3 | 620.2639 |
| 488 | 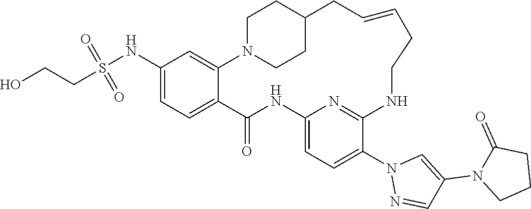 [SMILES: O=C1Nc2ccc(-n3cc(N4CCCC4=O)cn3)c(n2)NCC/C=C/CC2CCN(CC2)c2cc(NS(=O)(=O)CCO)ccc21] | 635.3 | 635.2748 |
| 489 | 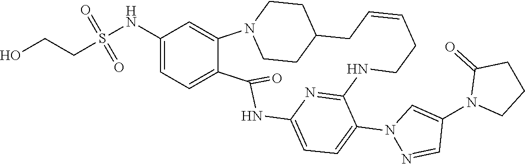 [SMILES: O=C1Nc2ccc(-n3cc(N4CCCC4=O)cn3)c(n2)NCC/C=C\CC2CCN(CC2)c2cc(NS(=O)(=O)CCO)ccc21] | 635.4 | 635.2761 |
| 490 | 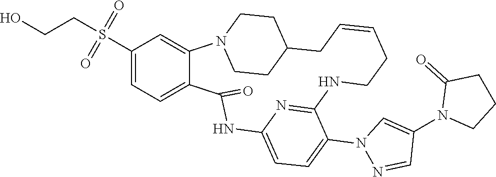 [SMILES: O=C1Nc2ccc(-n3cc(N4CCCC4=O)cn3)c(n2)NCC/C=C\CC2CCN(CC2)c2cc(S(=O)(=O)CCO)ccc21] | 620.4 | 620.2639 |
| 491 | 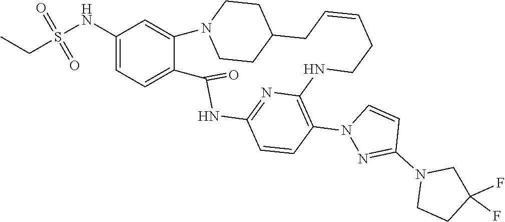 [SMILES: CCS(=O)(=O)Nc1ccc2c(c1)N1CCC(C/C=C\CCNc3nc(ccc3-n3ccc(N4CCC(F)(F)C4)n3)NC2=O)CC1] | 672.2 | 672.3043 |
| 492 | 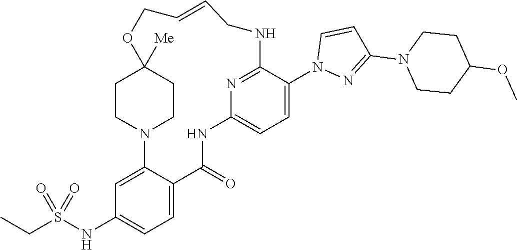 [SMILES: CCS(=O)(=O)Nc1ccc2c(c1)N1CCC(C)(CC1)OC/C=C/CNc1nc(ccc1-n1ccc(N3CCC(OC)CC3)n1)NC2=O] | 665.2 | 665.3212 |
| 493 | 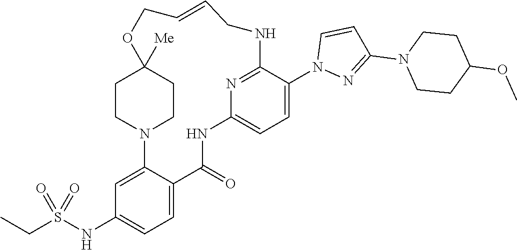 [SMILES: CCS(=O)(=O)Nc1ccc2c(c1)N1CCC(C)(CC1)OC/C=C/CNc1nc(ccc1-n1ccc(N3CCC(OC)CC3)n1)NC2=O] | 650.2 | 650.3212 |
| 494 | 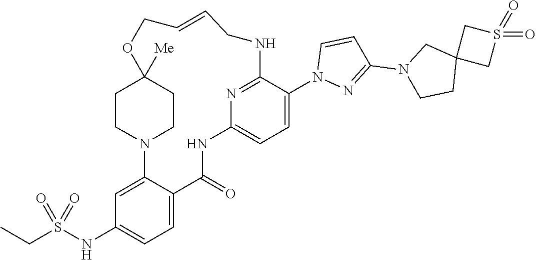 [SMILES: CCS(=O)(=O)Nc1ccc2c(c1)N1CCC(C)(CC1)OC/C=C/CNc1nc(ccc1-n1ccc(N3CCC4(C3)CS(=O)(=O)C4)n1)NC2=O] | 711.1 | 711.2724 |
| 495 | 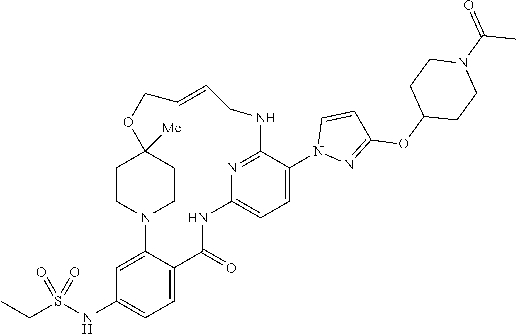 [SMILES: CCS(=O)(=O)Nc1ccc2c(c1)N1CCC(C)(CC1)OC/C=C/CNc1nc(ccc1-n1ccc(OC3CCN(C(C)=O)CC3)n1)NC2=O] | 693.3 | 693.3167 |
| 496 | 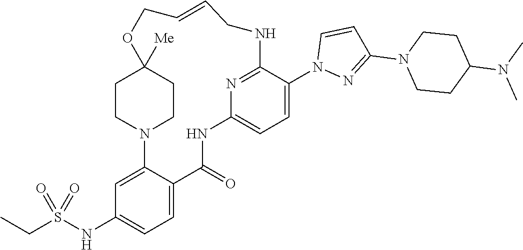 [SMILES: CCS(=O)(=O)Nc1ccc2c(c1)N1CCC(C)(CC1)OC/C=C/CNc1nc(ccc1-n1ccc(N3CCC(N(C)C)CC3)n1)NC2=O] | 678.3 | 678.3522 |
| 497 | 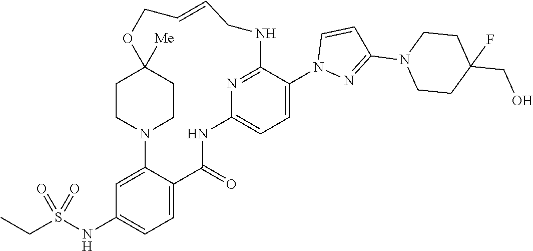 [SMILES: CCS(=O)(=O)Nc1ccc2c(c1)N1CCC(C)(CC1)OC/C=C/CNc1nc(ccc1-n1ccc(N3CCC(F)(CO)CC3)n1)NC2=O] | 683.3 | 683.311 |
| 498 | 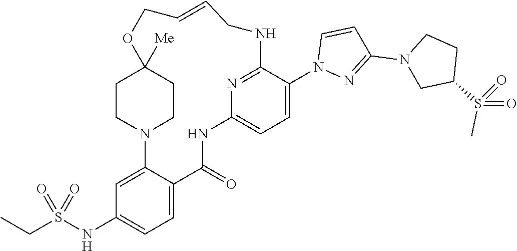 [SMILES: CCS(=O)(=O)Nc1ccc2c(c1)N1CCC(C)(CC1)OC/C=C/CNc1nc(ccc1-n1ccc(N3CC[C@H](S(C)(=O)=O)C3)n1)NC2=O] | 699.2 | 699.2719 |
| 499 | 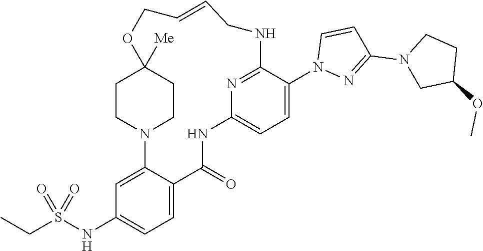 [SMILES: CCS(=O)(=O)Nc1ccc2c(c1)N1CCC(C)(CC1)OC/C=C/CNc1nc(ccc1-n1ccc(N3CC[C@@H](OC)C3)n1)NC2=O] | 651.2 | 651.3051 |
| 500 | 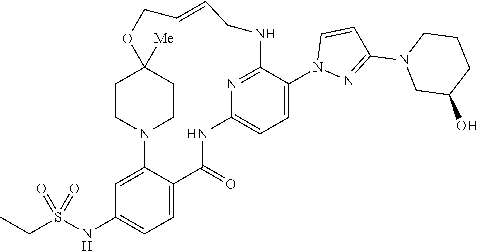 [SMILES: CCS(=O)(=O)Nc1ccc2c(c1)N1CCC(C)(CC1)OC/C=C/CNc1nc(ccc1-n1ccc(N3CCC[C@@H](O)C3)n1)NC2=O] | 651.3 | 651.3048 |
| 501 | 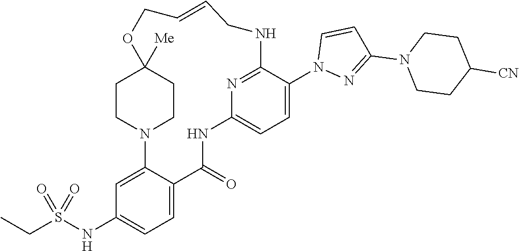 [SMILES: CCS(=O)(=O)Nc1ccc2c(c1)N1CCC(C)(CC1)OC/C=C/CNc1nc(ccc1-n1ccc(N3CCC(C#N)CC3)n1)NC2=O] | 660.2 | 660.3062 |
| 502 | 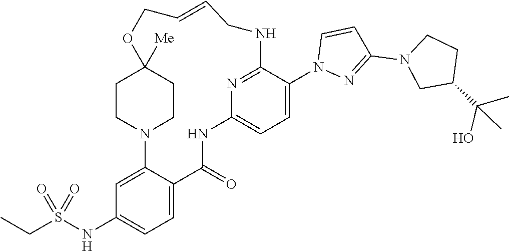 [SMILES: CCS(=O)(=O)Nc1ccc2c(c1)N1CCC(C)(CC1)OC/C=C/CNc1nc(ccc1-n1ccc(N3CC[C@H](C(C)(C)O)C3)n1)NC2=O] | 679.2 | 679.3373 |
| 503 | 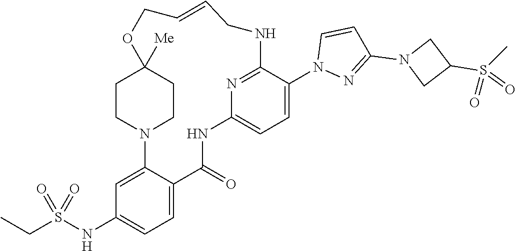 [SMILES: CCS(=O)(=O)Nc1ccc2c(c1)N1CCC(C)(CC1)OC/C=C/CNc1nc(ccc1-n1ccc(N3CC(S(C)(=O)=O)C3)n1)NC2=O] | 685.2 | 685.2568 |
| 504 | 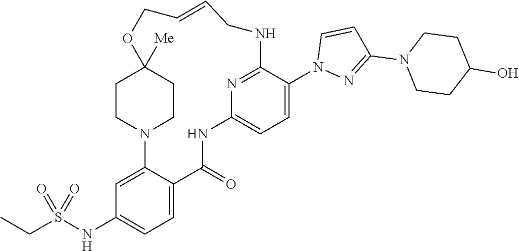 [SMILES: CCS(=O)(=O)Nc1ccc2c(c1)N1CCC(C)(CC1)OC/C=C/CNc1nc(ccc1-n1ccc(N3CCC(O)CC3)n1)NC2=O] | 651.3 | 651.3047 |
| 505 | 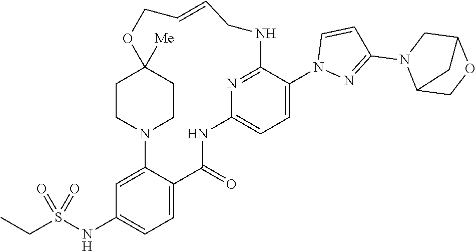 [SMILES: CCS(=O)(=O)Nc1ccc2c(c1)N1CCC(C)(CC1)OC/C=C/CNc1nc(ccc1-n1ccc(N3CC4CC3CO4)n1)NC2=O] | 649.0 | 649.2901 |
| 506 | 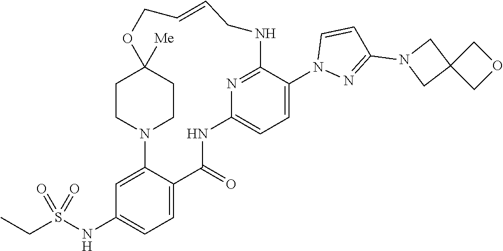 [SMILES: CCS(=O)(=O)Nc1ccc2c(c1)N1CCC(C)(CC1)OC/C=C/CNc1nc(ccc1-n1ccc(N3CC4(COC4)C3)n1)NC2=O] | 649.1 | 649.2905 |
| 507 | 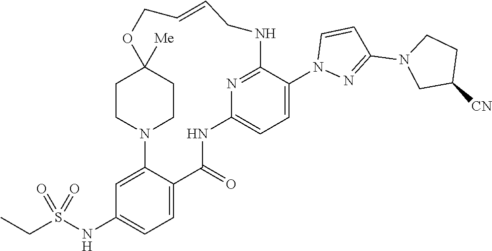 [SMILES: CCS(=O)(=O)Nc1ccc2c(c1)N1CCC(C)(CC1)OC/C=C/CNc1nc(ccc1-n1ccc(N3CC[C@@H](C#N)C3)n1)NC2=O] | 646.2 | 646.29 |
| 508 | 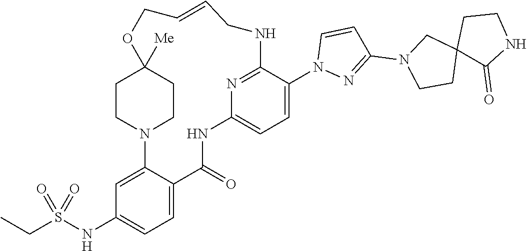 [SMILES: CCS(=O)(=O)Nc1ccc2c(c1)N1CCC(C)(CC1)OC/C=C/CNc1nc(ccc1-n1ccc(N3CCC4(CCNC4=O)C3)n1)NC2=O] | 690.4 | 690.3162 |
| 509 | 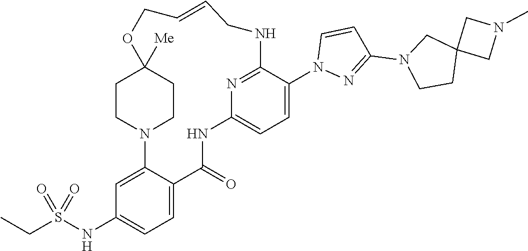 [SMILES: CCS(=O)(=O)Nc1ccc2c(c1)N1CCC(C)(CC1)OC/C=C/CNc1nc(ccc1-n1ccc(N3CCC4(CN(C)C4)C3)n1)NC2=O] | 676.3 | 676.3378 |
| 510 | 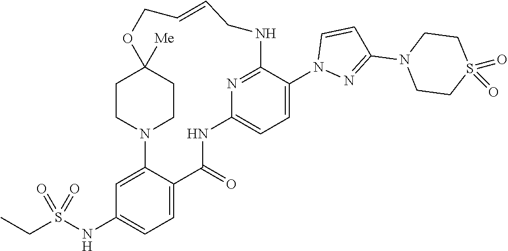 [SMILES: CCS(=O)(=O)Nc1ccc2c(c1)N1CCC(C)(CC1)OC/C=C/CNc1nc(ccc1-n1ccc(N3CCS(=O)(=O)CC3)n1)NC2=O] | 685.2 | 685.256 |
| 511 | 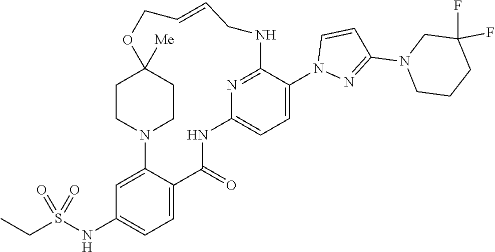 [SMILES: CCS(=O)(=O)Nc1ccc2c(c1)N1CCC(C)(CC1)OC/C=C/CNc1nc(ccc1-n1ccc(N3CCCC(F)(F)C3)n1)NC2=O] | 671.2 | 671.2918 |
| 512 | 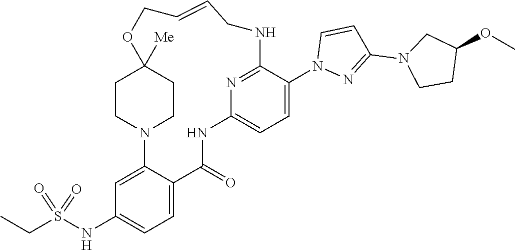 [SMILES: CCS(=O)(=O)Nc1ccc2c(c1)N1CCC(C)(CC1)OC/C=C/CNc1nc(ccc1-n1ccc(N3CC[C@H](OC)C3)n1)NC2=O] | 651.2 | 651.3053 |
| 513 | 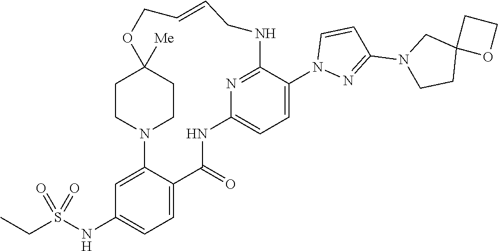 [SMILES: CCS(=O)(=O)Nc1ccc2c(c1)N1CCC(C)(CC1)OC/C=C/CNc1nc(ccc1-n1ccc(N3CCC4(CCO4)C3)n1)NC2=O] | 663.2 | 663.3054 |
| 514 | 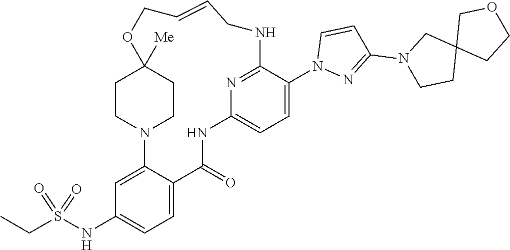 [SMILES: CCS(=O)(=O)Nc1ccc2c(c1)N1CCC(C)(CC1)OC/C=C/CNc1nc(ccc1-n1ccc(N3CCC4(CCOC4)C3)n1)NC2=O] | 677.3 | 677.3219 |
| 515 | 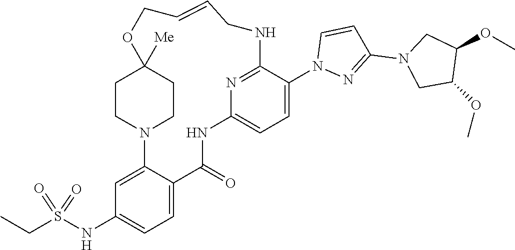 [SMILES: CCS(=O)(=O)Nc1ccc2c(c1)N1CCC(C)(CC1)OC/C=C/CNc1nc(ccc1-n1ccc(N3C[C@@H](OC)[C@H](OC)C3)n1)NC2=O] | 681.3 | 681.3166 |
| 516 | 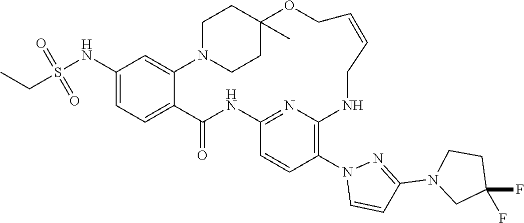 | 657.2 | 657.2764 |
| 517 | 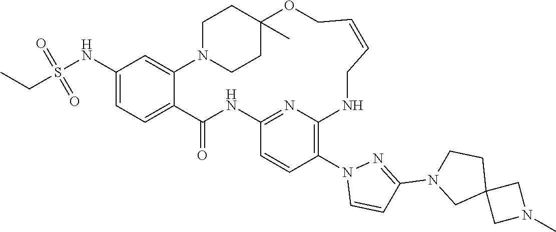 [SMILES: CCS(=O)(=O)Nc1ccc2c(c1)N1CCC(C)(CC1)OC/C=C\CNc1nc(ccc1-n1ccc(N3CCC4(CN(C)C4)C3)n1)NC2=O] | 676.2 | 676.3374 |
| 518 | 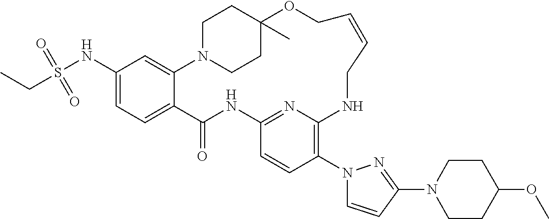 [SMILES: CCS(=O)(=O)Nc1ccc2c(c1)N1CCC(C)(CC1)OC/C=C\CNc1nc(ccc1-n1ccc(N3CCC(OC)CC3)n1)NC2=O] | 665.2 | 665.3204 |
| 519 | 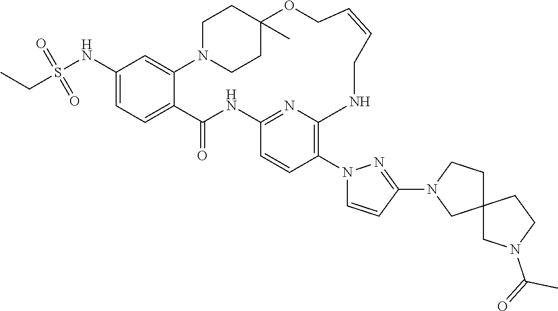 [SMILES: CCS(=O)(=O)Nc1ccc2c(c1)N1CCC(C)(CC1)OC/C=C\CNc1nc(ccc1-n1ccc(N3CCC4(CCN(C(C)=O)C4)C3)n1)NC2=O] | 718.3 | 718.3479 |
| 520 | 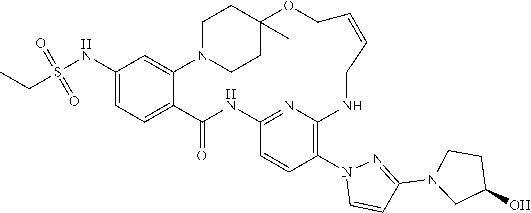 [SMILES: CCS(=O)(=O)Nc1ccc2c(c1)N1CCC(C)(CC1)OC/C=C\CNc1nc(ccc1-n1ccc(N3CC[C@@H](O)C3)n1)NC2=O] | 637.2 | 637.2884 |
| 521 | 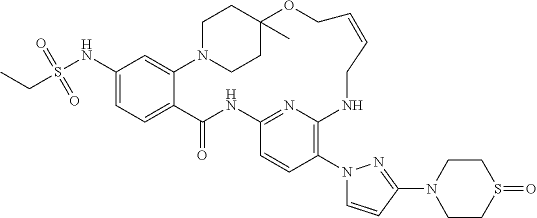 [SMILES: CCS(=O)(=O)Nc1ccc2c(c1)N1CCC(C)(CC1)OC/C=C\CNc1nc(ccc1-n1ccc(N3CCS(=O)CC3)n1)NC2=O] | 669.1 | 669.2624 |
| 522 | 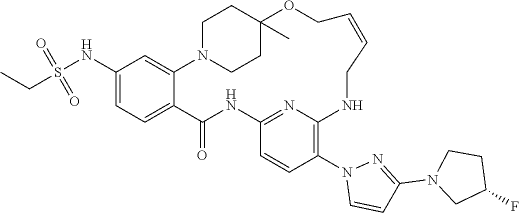 [SMILES: CCS(=O)(=O)Nc1ccc2c(c1)N1CCC(C)(CC1)OC/C=C\CNc1nc(ccc1-n1ccc(N3CC[C@H](F)C3)n1)NC2=O] | 639.2 | 639.2844 |
| 523 | 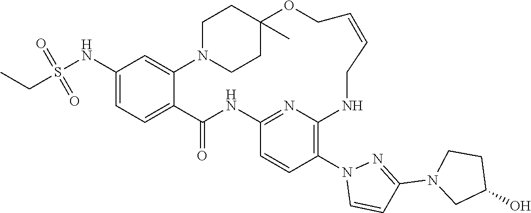 [SMILES: CCS(=O)(=O)Nc1ccc2c(c1)N1CCC(C)(CC1)OC/C=C\CNc1nc(ccc1-n1ccc(N3CC[C@H](O)C3)n1)NC2=O] | 637.1 | 637.2899 |
| 524 | 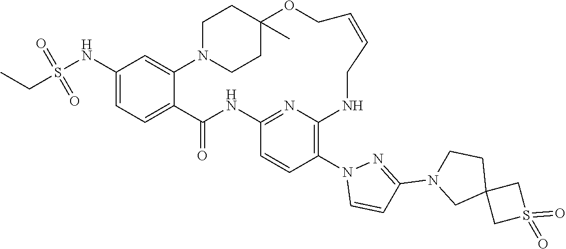 [SMILES: CCS(=O)(=O)Nc1ccc2c(c1)N1CCC(C)(CC1)OC/C=C\CNc1nc(ccc1-n1ccc(N3CCC4(C3)CS(=O)(=O)C4)n1)NC2=O] | 711.2 | 711.2721 |
| 525 | 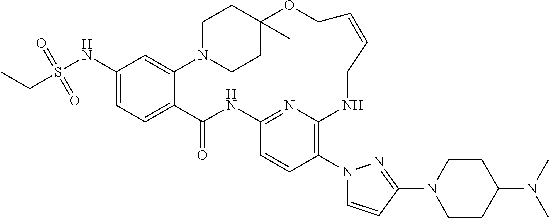 [SMILES: CCS(=O)(=O)Nc1ccc2c(c1)N1CCC(C)(CC1)OC/C=C\CNc1nc(ccc1-n1ccc(N3CCC(N(C)C)CC3)n1)NC2=O] | 678.2 | 678.3536 |
| 526 | 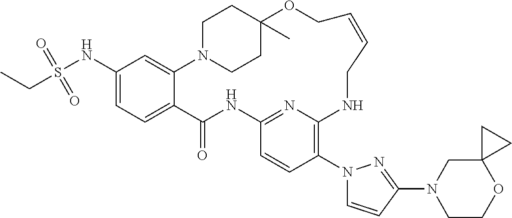 [SMILES: CCS(=O)(=O)Nc1ccc2c(c1)N1CCC(C)(CC1)OC/C=C\CNc1nc(ccc1-n1ccc(N3CCOC4(CC4)C3)n1)NC2=O] | 663.2 | 663.3057 |
| 527 | 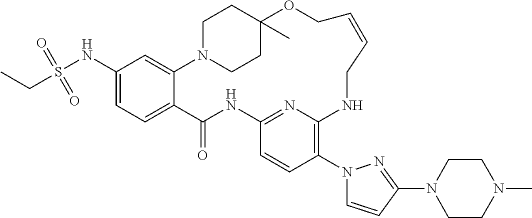 [SMILES: CCS(=O)(=O)Nc1ccc2c(c1)N1CCC(C)(CC1)OC/C=C\CNc1nc(ccc1-n1ccc(N3CCN(C)CC3)n1)NC2=O] | 650.2 | 650.3222 |
| 528 | 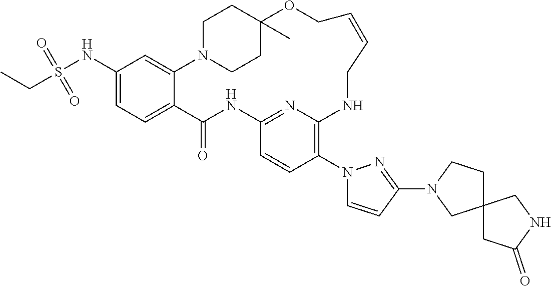 [SMILES: CCS(=O)(=O)Nc1ccc2c(c1)N1CCC(C)(CC1)OC/C=C\CNc1nc(ccc1-n1ccc(N3CCC4(CNC(=O)C4)C3)n1)NC2=O] | 690.1 | 690.3168 |
| 529 | 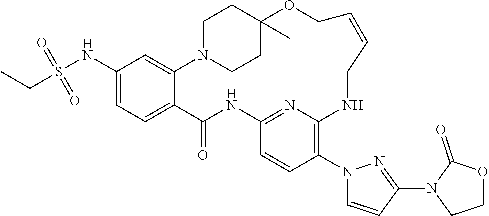 [SMILES: CCS(=O)(=O)Nc1ccc2c(c1)N1CCC(C)(CC1)OC/C=C\CNc1nc(ccc1-n1ccc(N3CCOC3=O)n1)NC2=O] | 637.2 | 637.2537 |
| 530 | 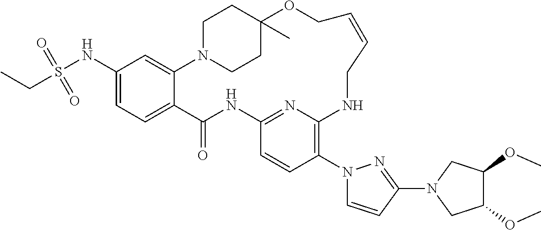 [SMILES: CCS(=O)(=O)Nc1ccc2c(c1)N1CCC(C)(CC1)OC/C=C\CNc1nc(ccc1-n1ccc(N3C[C@@H](OC)[C@H](OC)C3)n1)NC2=O] | 681.2 | 681.3156 |
| 531 | 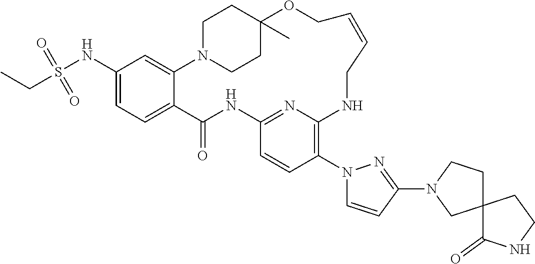 [SMILES: CCS(=O)(=O)Nc1ccc2c(c1)N1CCC(C)(CC1)OC/C=C\CNc1nc(ccc1-n1ccc(N3CCC4(CCNC4=O)C3)n1)NC2=O] | 690.2 | 690.3146 |
| 532 | 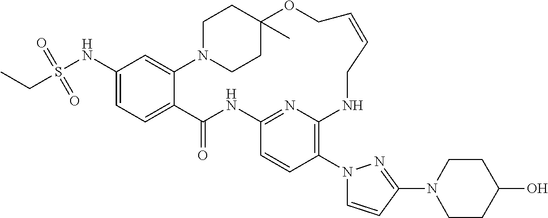 [SMILES: CCS(=O)(=O)Nc1ccc2c(c1)N1CCC(C)(CC1)OC/C=C\CNc1nc(ccc1-n1ccc(N3CCC(O)CC3)n1)NC2=O] | 651.3 | 651.3041 |
| 533 | 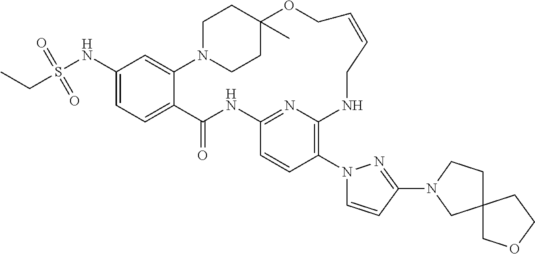 [SMILES: CCS(=O)(=O)Nc1ccc2c(c1)N1CCC(C)(CC1)OC/C=C\CNc1nc(ccc1-n1ccc(N3CCC4(CCOC4)C3)n1)NC2=O] | 677.2 | 677.3211 |
| 534 | 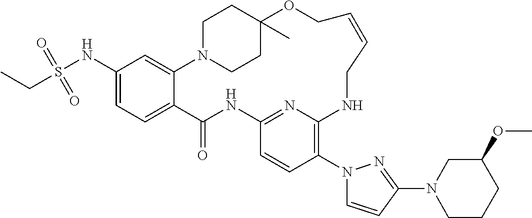 [SMILES: CCS(=O)(=O)Nc1ccc2c(c1)N1CCC(C)(CC1)OC/C=C\CNc1nc(ccc1-n1ccc(N3CCC[C@H](OC)C3)n1)NC2=O] | 665.2 | 665.3205 |
| 535 | 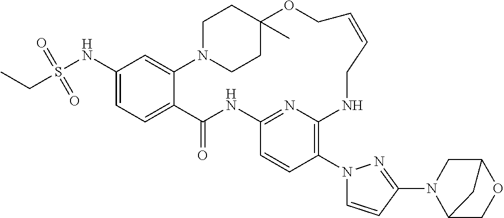 [SMILES: CCS(=O)(=O)Nc1ccc2c(c1)N1CCC(C)(CC1)OC/C=C\CNc1nc(ccc1-n1ccc(N3CC4CC3CO4)n1)NC2=O] | 649.1 | 649.2891 |
| 536 | 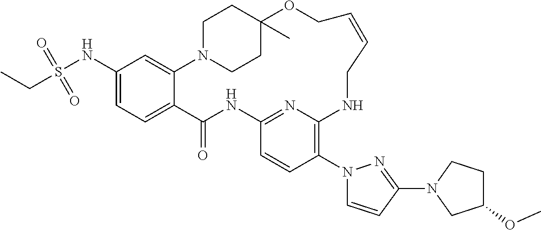 [SMILES: CCS(=O)(=O)Nc1ccc2c(c1)N1CCC(C)(CC1)OC/C=C\CNc1nc(ccc1-n1ccc(N3CC[C@H](OC)C3)n1)NC2=O] | 651.2 | 651.305 |
| 537 | 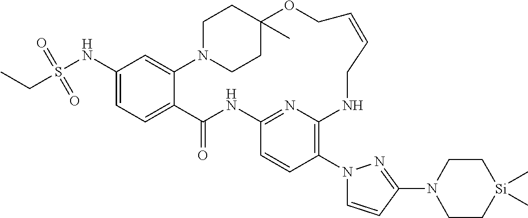 [SMILES: CCS(=O)(=O)Nc1ccc2c(c1)N1CCC(C)(CC1)OC/C=C\CNc1nc(ccc1-n1ccc(N3CC[Si](C)(C)CC3)n1)NC2=O] | 679.2 | 679.3187 |
| 538 | 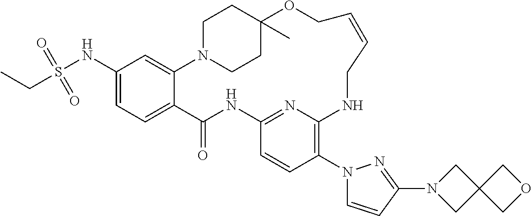 [SMILES: CCS(=O)(=O)Nc1ccc2c(c1)N1CCC(C)(CC1)OC/C=C\CNc1nc(ccc1-n1ccc(N3CC4(COC4)C3)n1)NC2=O] | 649.1 | 649.2902 |
| 539 | 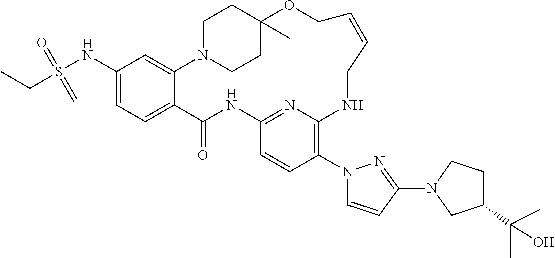 [SMILES: C=S(=O)(CC)Nc1ccc2c(c1)N1CCC(C)(CC1)OC/C=C\CNc1nc(ccc1-n1ccc(N3CC[C@H](C(C)(C)O)C3)n1)NC2=O] | 679.2 | 679.3359 |
| 540 | 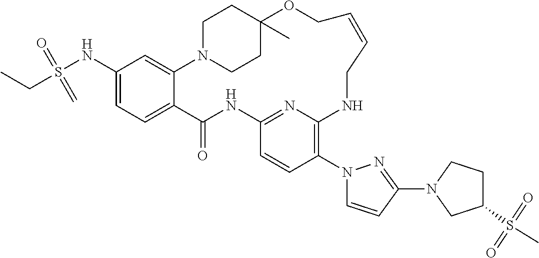 [SMILES: C=S(=O)(CC)Nc1ccc2c(c1)N1CCC(C)(CC1)OC/C=C\CNc1nc(ccc1-n1ccc(N3CC[C@H](S(C)(=O)=O)C3)n1)NC2=O] | 699.1 | 699.2722 |
| 541 | 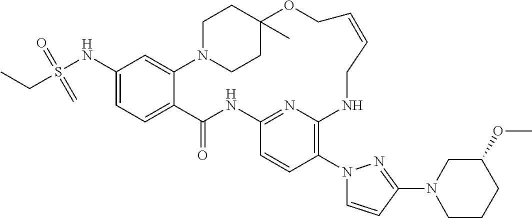 [SMILES: C=S(=O)(CC)Nc1ccc2c(c1)N1CCC(C)(CC1)OC/C=C\CNc1nc(ccc1-n1ccc(N3CCC[C@@H](OC)C3)n1)NC2=O] | 665.2 | 665.3216 |
| 542 | 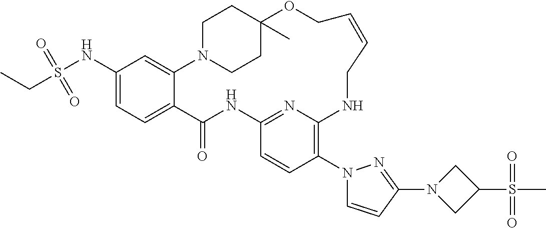 [SMILES: CCS(=O)(=O)Nc1ccc2c(c1)N1CCC(C)(CC1)OC/C=C\CNc1nc(ccc1-n1ccc(N3CC(S(C)(=O)=O)C3)n1)NC2=O] | 685.2 | 685.257 |
| 543 | 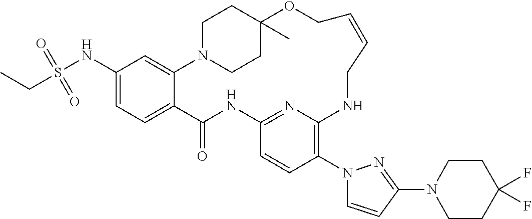 [SMILES: CCS(=O)(=O)Nc1ccc2c(c1)N1CCC(C)(CC1)OC/C=C\CNc1nc(ccc1-n1ccc(N3CCC(F)(F)CC3)n1)NC2=O] | 671.2 | 671.2922 |
| 544 | 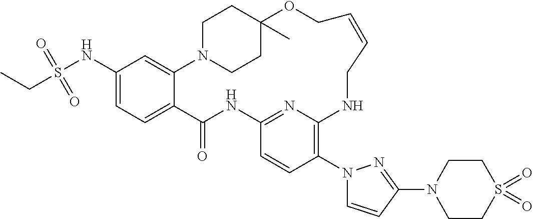 [SMILES: CCS(=O)(=O)Nc1ccc2c(c1)N1CCC(C)(CC1)OC/C=C\CNc1nc(ccc1-n1ccc(N3CCS(=O)(=O)CC3)n1)NC2=O] | 685.2 | 685.2568 |
| 545 | 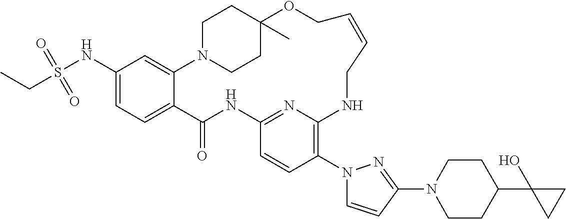 [SMILES: CCS(=O)(=O)Nc1ccc2c(c1)N1CCC(C)(CC1)OC/C=C\CNc1nc(ccc1-n1ccc(N3CCC(C4(O)CC4)CC3)n1)NC2=O] | 691.2 | 691.3373 |
| 546 | 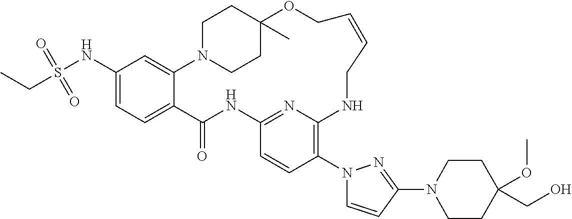 [SMILES: CCS(=O)(=O)Nc1ccc2c(c1)N1CCC(C)(CC1)OC/C=C\CNc1nc(ccc1-n1ccc(N3CCC(CO)(OC)CC3)n1)NC2=O] | 695.2 | 695.3332 |
| 547 | 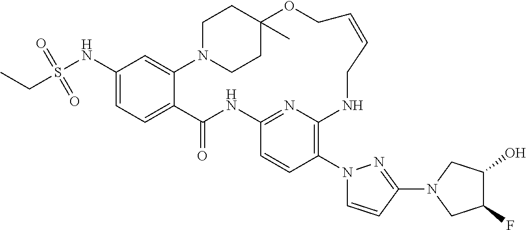 [SMILES: CCS(=O)(=O)Nc1ccc2c(c1)N1CCC(C)(CC1)OC/C=C\CNc1nc(ccc1-n1ccc(N3C[C@H](O)[C@@H](F)C3)n1)NC2=O] | 655.1 | 655.2804 |
| 548 | 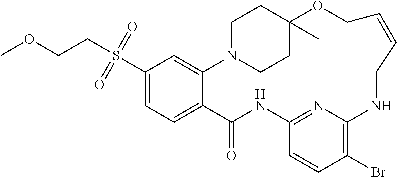 [SMILES: COCCS(=O)(=O)c1ccc2c(c1)N1CCC(C)(CC1)OC/C=C\CNc1nc(ccc1Br)NC2=O] | 581.0 | 579.1254 |
| 549 | 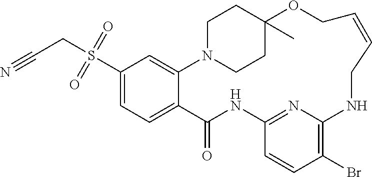 [SMILES: CC12CCN(CC1)c1cc(S(=O)(=O)CC#N)ccc1C(=O)Nc1ccc(Br)c(n1)NC/C=C\CO2] | 562.1 | 560.095 |
| 550 | 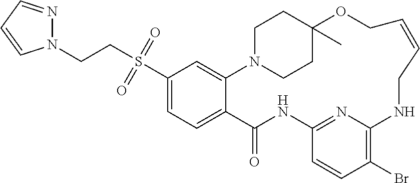 [SMILES: CC12CCN(CC1)c1cc(S(=O)(=O)CCn3cccn3)ccc1C(=O)Nc1ccc(Br)c(n1)NC/C=C\CO2] | 617.0 | 615.1377 |
| 551 | 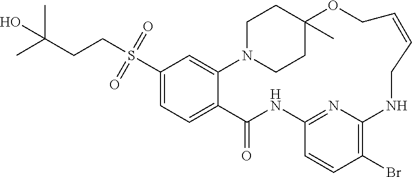 [SMILES: CC(C)(O)CCS(=O)(=O)c1ccc2c(c1)N1CCC(C)(CC1)OC/C=C\CNc1nc(ccc1Br)NC2=O] | 609.2 | 607.1563 |
| 552 | 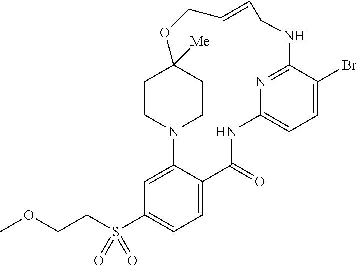 [SMILES: COCCS(=O)(=O)c1ccc2c(c1)N1CCC(C)(CC1)OC/C=C/CNc1nc(ccc1Br)NC2=O] | 581.1 | 579.1258 |
| 553 | 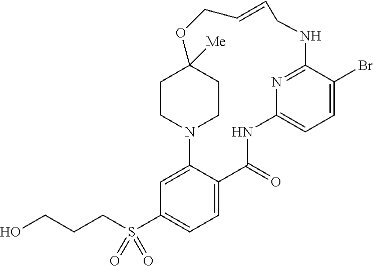 [SMILES: CC12CCN(CC1)c1cc(S(=O)(=O)CCCO)ccc1C(=O)Nc1ccc(Br)c(n1)NC/C=C/CO2] | 581.1 | 579.1254 |
| 554 | 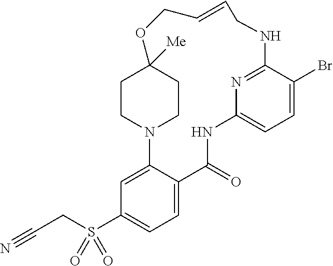 [SMILES: CC12CCN(CC1)c1cc(S(=O)(=O)CC#N)ccc1C(=O)Nc1ccc(Br)c(n1)NC/C=C/CO2] | 562.0 | 560.0959 |
| 555 | 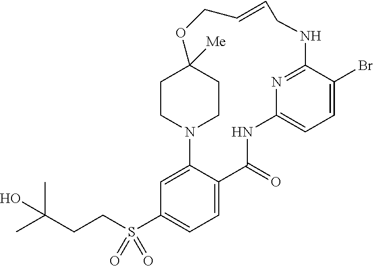 [SMILES: CC(C)(O)CCS(=O)(=O)c1ccc2c(c1)N1CCC(C)(CC1)OC/C=C/CNc1nc(ccc1Br)NC2=O] | 617.0 | 615.1378 |
| 556 | 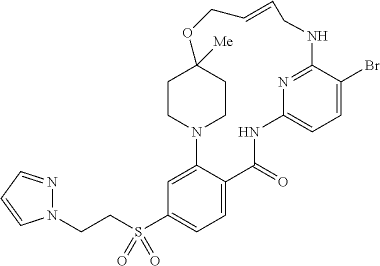 [SMILES: CC12CCN(CC1)c1cc(S(=O)(=O)CCn3cccn3)ccc1C(=O)Nc1ccc(Br)c(n1)NC/C=C/CO2] | 608.9 | 607.1576 |
| 557 | 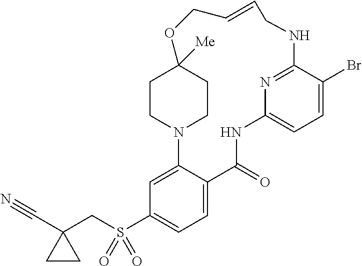 [SMILES: CC12CCN(CC1)c1cc(S(=O)(=O)CC3(C#N)CC3)ccc1C(=O)Nc1ccc(Br)c(n1)NC/C=C/CO2] | 602.0 | 600.1261 |
| 558 | 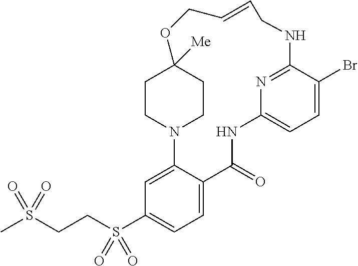 [SMILES: CC12CCN(CC1)c1cc(S(=O)(=O)CCS(C)(=O)=O)ccc1C(=O)Nc1ccc(Br)c(n1)NC/C=C/CO2] | 629.0 | 627.0917 |
| 559 | 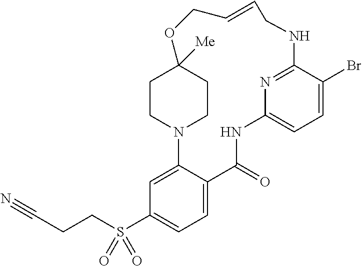 [SMILES: CC12CCN(CC1)c1cc(S(=O)(=O)CCC#N)ccc1C(=O)Nc1ccc(Br)c(n1)NC/C=C/CO2] | 576.1 | 574.1102 |
| 560 | 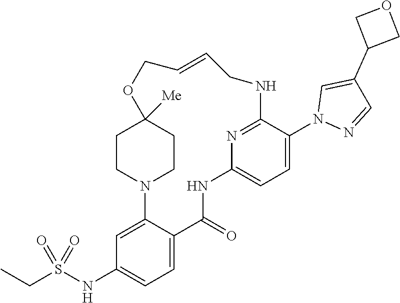 [SMILES: CCS(=O)(=O)Nc1ccc2c(c1)N1CCC(C)(CC1)OC/C=C/CNc1nc(ccc1-n1cc(C3COC3)cn1)NC2=O] | 608.3 | 608.2636 |
| 561 | 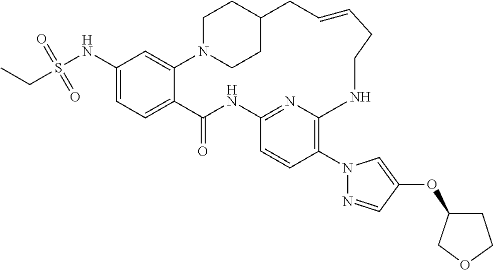 [SMILES: CCS(=O)(=O)Nc1ccc2c(c1)N1CCC(C/C=C/CCNc3nc(ccc3-n3cc(O[C@H]4CCOC4)cn3)NC2=O)CC1] | 622.4 | 622.279 |
| 562 | 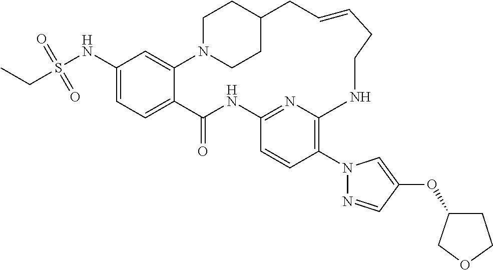 [SMILES: CCS(=O)(=O)Nc1ccc2c(c1)N1CCC(C/C=C/CCNc3nc(ccc3-n3cc(O[C@@H]4CCOC4)cn3)NC2=O)CC1] | 622.4 | 622.2792 |
| 563 | 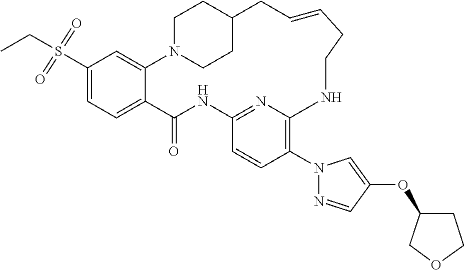 [SMILES: CCS(=O)(=O)c1ccc2c(c1)N1CCC(C/C=C/CCNc3nc(ccc3-n3cc(O[C@H]4CCOC4)cn3)NC2=O)CC1] | 607.3 | 607.2681 |
| 564 | 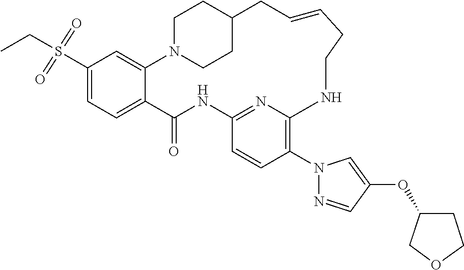 [SMILES: CCS(=O)(=O)c1ccc2c(c1)N1CCC(C/C=C/CCNc3nc(ccc3-n3cc(O[C@@H]4CCOC4)cn3)NC2=O)CC1] | 607.4 | 607.2689 |
| 565 | 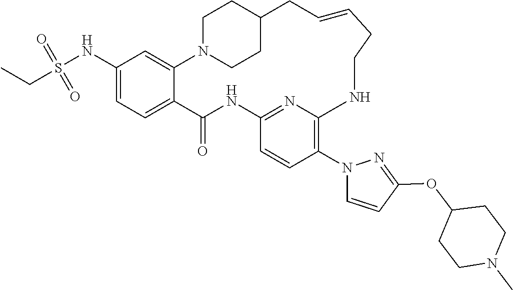 [SMILES: CCS(=O)(=O)Nc1ccc2c(c1)N1CCC(C/C=C/CCNc3nc(ccc3-n3ccc(OC4CCN(C)CC4)n3)NC2=O)CC1] | 649.1 | 649.3275 |
| 566 | 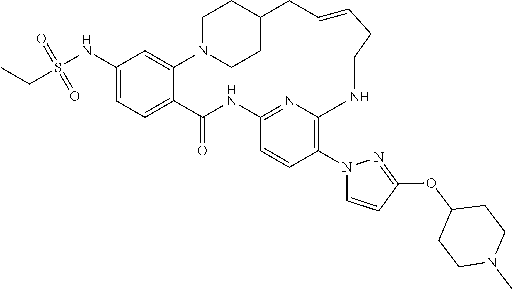 [SMILES: CCS(=O)(=O)Nc1ccc2c(c1)N1CCC(C/C=C/CCNc3nc(ccc3-n3ccc(OC4CCN(C)CC4)n3)NC2=O)CC1] | 677.2 | 677.3228 |
| 567 | 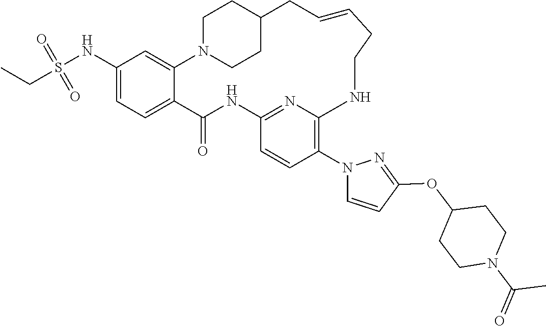 [SMILES: CCS(=O)(=O)Nc1ccc2c(c1)N1CCC(C/C=C/CCNc3nc(ccc3-n3ccc(OC4CCN(C(C)=O)CC4)n3)NC2=O)CC1] | 606.2 | 606.2856 |
| 568 | 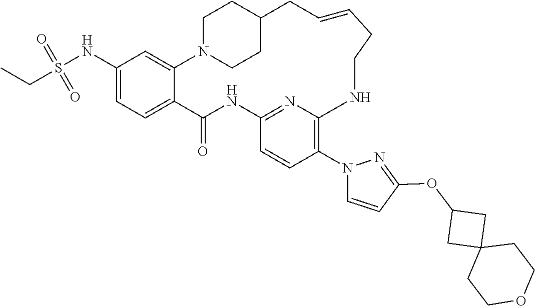 [SMILES: CCS(=O)(=O)Nc1ccc2c(c1)N1CCC(C/C=C/CCNc3nc(ccc3-n3ccc(OC4CC5(CCOCC5)C4)n3)NC2=O)CC1] | 676.3 | 676.3266 |
| 569 | 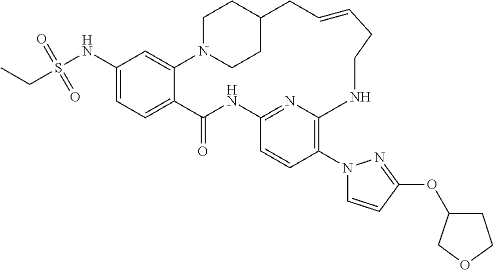 [SMILES: CCS(=O)(=O)Nc1ccc2c(c1)N1CCC(C/C=C/CCNc3nc(ccc3-n3ccc(OC4CCOC4)n3)NC2=O)CC1] | 622.2 | 622.2798 |
| 570 | 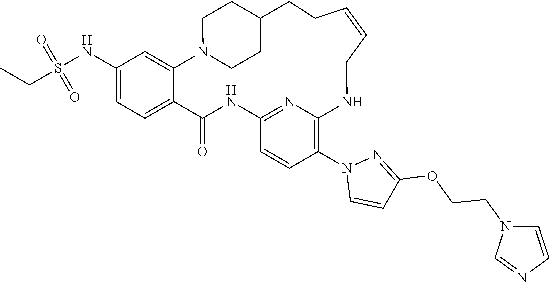 [SMILES: CCS(=O)(=O)Nc1ccc2c(c1)N1CCC(CC/C=C\CNc3nc(ccc3-n3ccc(OCCn4ccnc4)n3)NC2=O)CC1] | 646.2 | 646.2907 |
| 571 | 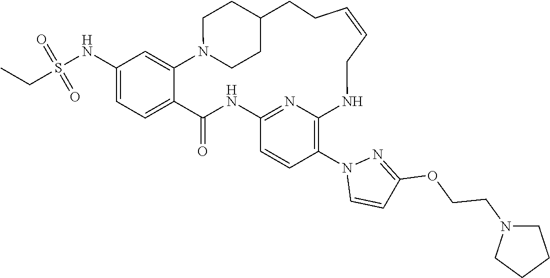 [SMILES: CCS(=O)(=O)Nc1ccc2c(c1)N1CCC(CC/C=C\CNc3nc(ccc3-n3ccc(OCCN4CCCC4)n3)NC2=O)CC1] | 649.2 | 649.327 |
| 572 | 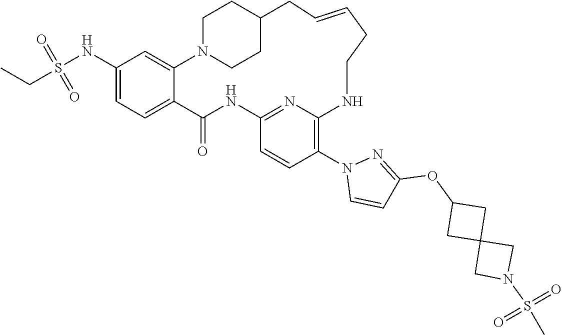 [SMILES: CCS(=O)(=O)Nc1ccc2c(c1)N1CCC(C/C=C/CCNc3nc(ccc3-n3ccc(OC4CC5(C4)CN(S(C)(=O)=O)C5)n3)NC2=O)CC1] | 725.0 | 725.2879 |
| 573 | 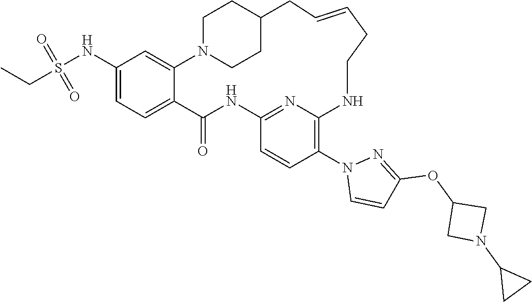 [SMILES: CCS(=O)(=O)Nc1ccc2c(c1)N1CCC(C/C=C/CCNc3nc(ccc3-n3ccc(OC4CN(C5CC5)C4)n3)NC2=O)CC1] | 647.3 | 647.3116 |
| 574 | 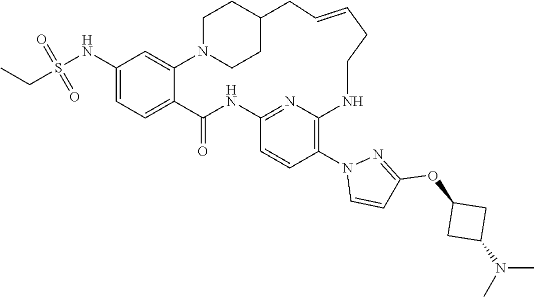 [SMILES: CCS(=O)(=O)Nc1ccc2c(c1)N1CCC(C/C=C/CCNc3nc(ccc3-n3ccc(O[C@H]4C[C@H](N(C)C)C4)n3)NC2=O)CC1] | 649.3 | 649.3271 |
| 575 | 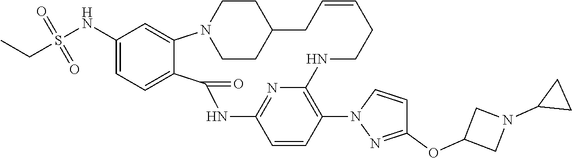 [SMILES: CCS(=O)(=O)Nc1ccc2c(c1)N1CCC(C/C=C\CCNc3nc(ccc3-n3ccc(OC4CN(C5CC5)C4)n3)NC2=O)CC1] | 647.2 | 647.3112 |
| 576 | 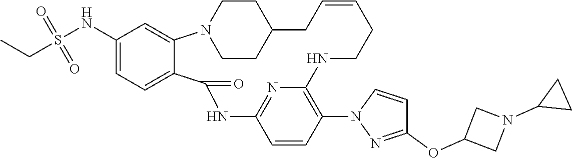 [SMILES: CCS(=O)(=O)Nc1ccc2c(c1)N1CCC(C/C=C\CCNc3nc(ccc3-n3ccc(OC4CN(C5CC5)C4)n3)NC2=O)CC1] | 606.1 | 606.285 |
| 577 | 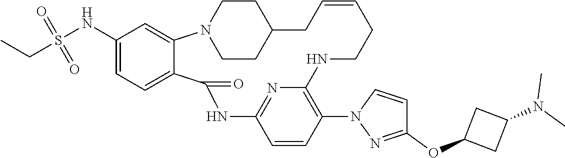 [SMILES: CCS(=O)(=O)Nc1ccc2c(c1)N1CCC(C/C=C\CCNc3nc(ccc3-n3ccc(O[C@H]4C[C@H](N(C)C)C4)n3)NC2=O)CC1] | 649.1 | 649.3265 |
| 578 | 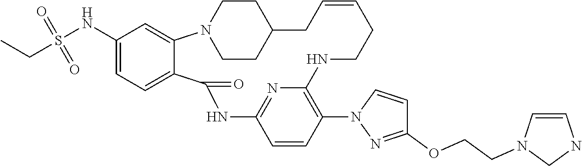 [SMILES: CCS(=O)(=O)Nc1ccc2c(c1)N1CCC(C/C=C\CCNc3nc(ccc3-n3ccc(OCCN4C=CNC4)n3)NC2=O)CC1] | 646.3 | 646.2911 |
| 579 | 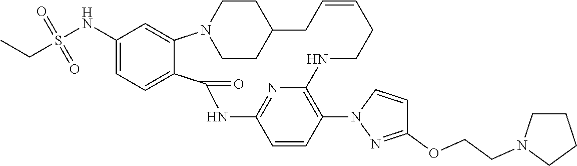 [SMILES: CCS(=O)(=O)Nc1ccc2c(c1)N1CCC(C/C=C\CCNc3nc(ccc3-n3ccc(OCCN4CCCC4)n3)NC2=O)CC1] | 649.1 | 649.3268 |
| 580 | 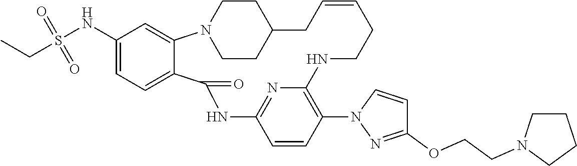 [SMILES: CCS(=O)(=O)Nc1ccc2c(c1)N1CCC(C/C=C\CCNc3nc(ccc3-n3ccc(OCCN4CCCC4)n3)NC2=O)CC1] | 649.1 | 649.3272 |
| 581 | 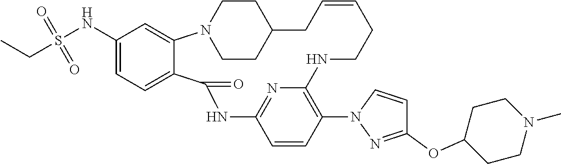 [SMILES: CCS(=O)(=O)Nc1ccc2c(c1)N1CCC(C/C=C\CCNc3nc(ccc3-n3ccc(OC4CCN(C)CC4)n3)NC2=O)CC1] | 677.3 | 677.3217 |
| 582 | 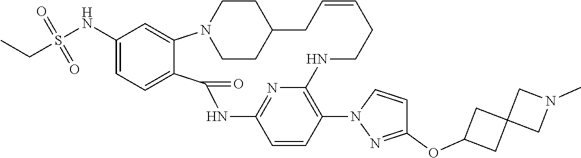 [SMILES: CCS(=O)(=O)Nc1ccc2c(c1)N1CCC(C/C=C\CCNc3nc(ccc3-n3ccc(OC4CC5(C4)CN(C)C5)n3)NC2=O)CC1] | 661.2 | 661.3276 |
| 583 | 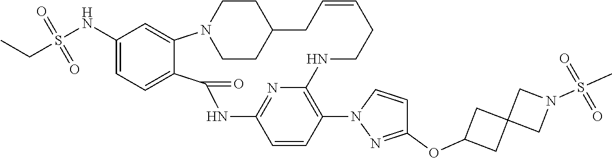 [SMILES: CCS(=O)(=O)Nc1ccc2c(c1)N1CCC(C/C=C\CCNc3nc(ccc3-n3ccc(OC4CC5(C4)CN(S(C)(=O)=O)C5)n3)NC2=O)CC1] | 725.1 | 725.2881 |
| 584 | 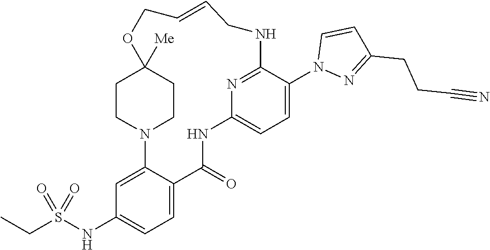 [SMILES: CCS(=O)(=O)Nc1ccc2c(c1)N1CCC(C)(CC1)OC/C=C/CNc1nc(ccc1-n1ccc(CCC#N)n1)NC2=O] | 605.1 | 605.2648 |
| 585 | 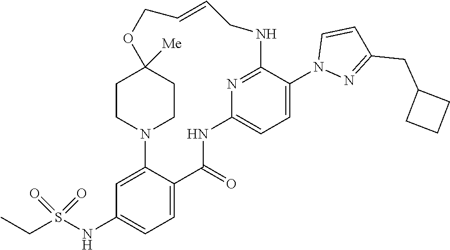 [SMILES: CCS(=O)(=O)Nc1ccc2c(c1)N1CCC(C)(CC1)OC/C=C/CNc1nc(ccc1-n1ccc(CC3CCC3)n1)NC2=O] | 620.2 | 620.3003 |
| 586 | 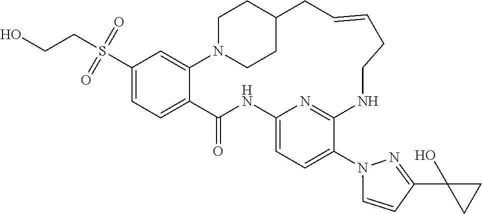 [SMILES: O=C1Nc2ccc(-n3ccc(C4(O)CC4)n3)c(n2)NCC/C=C/CC2CCN(CC2)c2cc(S(=O)(=O)CCO)ccc21] | 593.3 | 593.2519 |
| 587 | 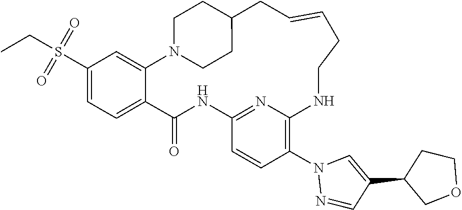 [SMILES: CCS(=O)(=O)c1ccc2c(c1)N1CCC(C/C=C/CCNc3nc(ccc3-n3cc([C@H]4CCOC4)cn3)NC2=O)CC1] | 591.3 | 591.2733 |
| 588 | 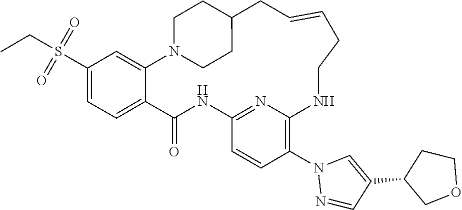 [SMILES: CCS(=O)(=O)c1ccc2c(c1)N1CCC(C/C=C/CCNc3nc(ccc3-n3cc([C@@H]4CCOC4)cn3)NC2=O)CC1] | 591.3 | 591.2724 |
| 589 | 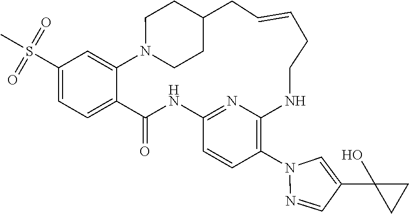 [SMILES: CS(=O)(=O)c1ccc2c(c1)N1CCC(C/C=C/CCNc3nc(ccc3-n3cc(C4(O)CC4)cn3)NC2=O)CC1] | 563.3 | 563.242 |
| 590 | 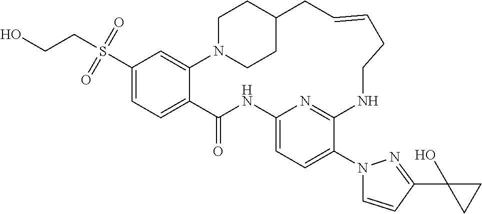 [SMILES: O=C1Nc2ccc(-n3ccc(C4(O)CC4)n3)c(n2)NCC/C=C/CC2CCN(CC2)c2cc(S(=O)(=O)CCO)ccc21] | | 593.2518 |
| 591 | 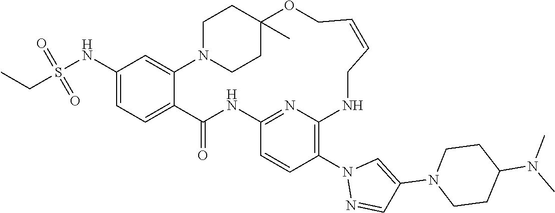 [SMILES: CCS(=O)(=O)Nc1ccc2c(c1)N1CCC(C)(CC1)OC/C=C\CNc1nc(ccc1-n1cc(N3CCC(N(C)C)CC3)cn1)NC2=O] | 678.3 | 678.3528 |
| 592 | 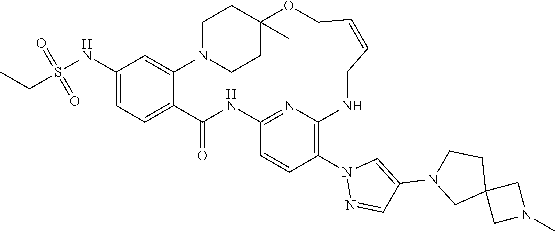 [SMILES: CCS(=O)(=O)Nc1ccc2c(c1)N1CCC(C)(CC1)OC/C=C\CNc1nc(ccc1-n1cc(N3CCC4(CN(C)C4)C3)cn1)NC2=O] | 676.3 | 676.3375 |
| 593 | 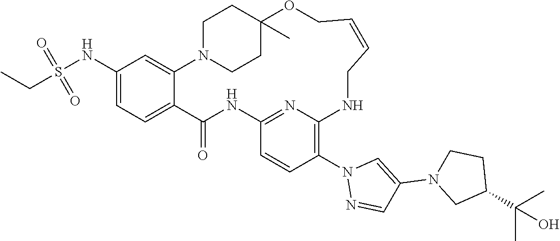 [SMILES: CCS(=O)(=O)Nc1ccc2c(c1)N1CCC(C)(CC1)OC/C=C\CNc1nc(ccc1-n1cc(N3CC[C@H](C(C)(C)O)C3)cn1)NC2=O] | 679.3 | 679.3356 |
| 594 | 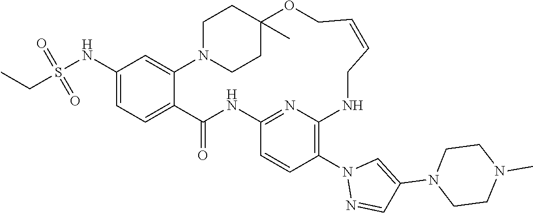 [SMILES: CCS(=O)(=O)Nc1ccc2c(c1)N1CCC(C)(CC1)OC/C=C\CNc1nc(ccc1-n1cc(N3CCN(C)CC3)cn1)NC2=O] | 650.2 | 650.3215 |
| 595 | 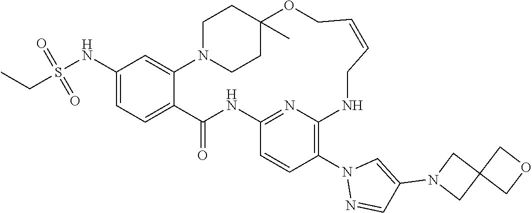 [SMILES: CCS(=O)(=O)Nc1ccc2c(c1)N1CCC(C)(CC1)OC/C=C\CNc1nc(ccc1-n1cc(N3CC4(COC4)C3)cn1)NC2=O] | 649.1 | 649.2896 |
| 596 | 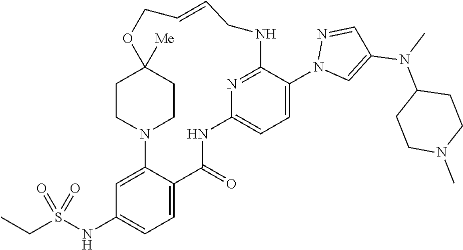 [SMILES: CCS(=O)(=O)Nc1ccc2c(c1)N1CCC(C)(CC1)OC/C=C/CNc1nc(ccc1-n1cc(N(C)C3CCN(C)CC3)cn1)NC2=O] | 678.2 | 678.3527 |
| 597 | 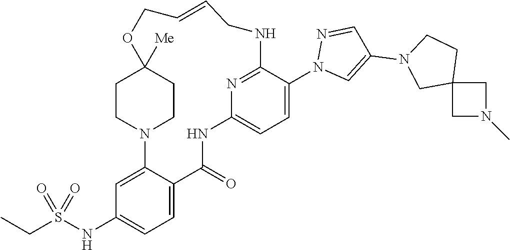 [SMILES: CCS(=O)(=O)Nc1ccc2c(c1)N1CCC(C)(CC1)OC/C=C/CNc1nc(ccc1-n1cc(N3CCC4(CN(C)C4)C3)cn1)NC2=O] | 676.2 | 676.3368 |
| 598 | 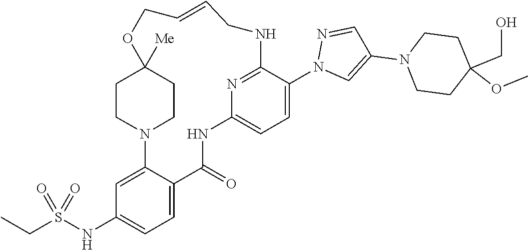 [SMILES: CCS(=O)(=O)Nc1ccc2c(c1)N1CCC(C)(CC1)OC/C=C/CNc1nc(ccc1-n1cc(N3CCC(CO)(OC)CC3)cn1)NC2=O] | 695.2 | 695.3304 |
| 599 | 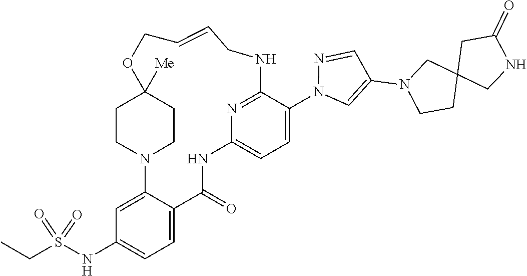 [SMILES: CCS(=O)(=O)Nc1ccc2c(c1)N1CCC(C)(CC1)OC/C=C/CNc1nc(ccc1-n1cc(N3CCC4(CNC(=O)C4)C3)cn1)NC2=O] | 690.2 | 690.3151 |
| 600 | 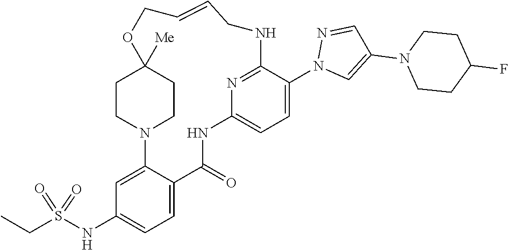 [SMILES: CCS(=O)(=O)Nc1ccc2c(c1)N1CCC(C)(CC1)OC/C=C/CNc1nc(ccc1-n1cc(N3CCC(F)CC3)cn1)NC2=O] | 653.2 | 653.2996 |
| 601 | 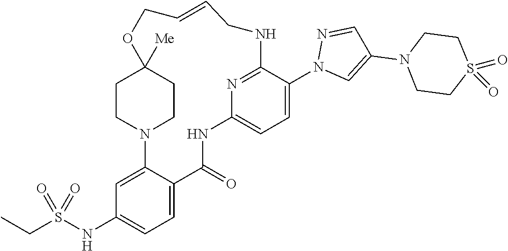 [SMILES: CCS(=O)(=O)Nc1ccc2c(c1)N1CCC(C)(CC1)OC/C=C/CNc1nc(ccc1-n1cc(N3CCS(=O)(=O)CC3)cn1)NC2=O] | 685.2 | 685.2567 |
| 602 | 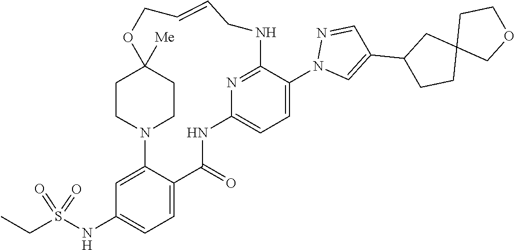 [SMILES: CCS(=O)(=O)Nc1ccc2c(c1)N1CCC(C)(CC1)OC/C=C/CNc1nc(ccc1-n1cc(C3CCC4(CCOC4)C3)cn1)NC2=O] | 677.2 | 677.3195 |
| 603 | 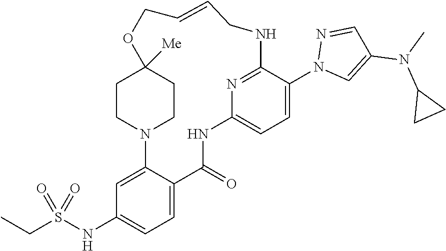 [SMILES: CCS(=O)(=O)Nc1ccc2c(c1)N1CCC(C)(CC1)OC/C=C/CNc1nc(ccc1-n1cc(N(C)C3CC3)cn1)NC2=O] | 621.2 | 621.2937 |
| 604 | 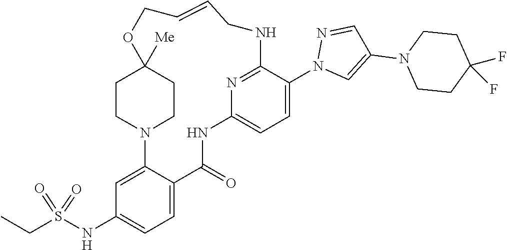 [SMILES: CCS(=O)(=O)Nc1ccc2c(c1)N1CCC(C)(CC1)OC/C=C/CNc1nc(ccc1-n1cc(N3CCC(F)(F)CC3)cn1)NC2=O] | 671.2 | 671.2907 |
| 605 | 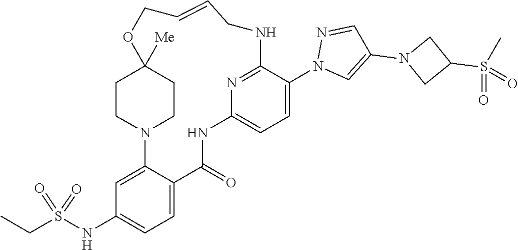 [SMILES: CCS(=O)(=O)Nc1ccc2c(c1)N1CCC(C)(CC1)OC/C=C/CNc1nc(ccc1-n1cc(N3CC(S(C)(=O)=O)C3)cn1)NC2=O] | 685.2 | 685.2558 |
| 606 | 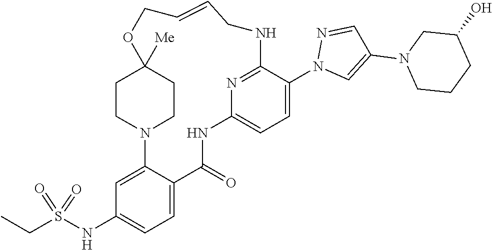 [SMILES: CCS(=O)(=O)Nc1ccc2c(c1)N1CCC(C)(CC1)OC/C=C/CNc1nc(ccc1-n1cc(N3CCC[C@@H](O)C3)cn1)NC2=O] | 651.2 | 651.3045 |
| 607 | 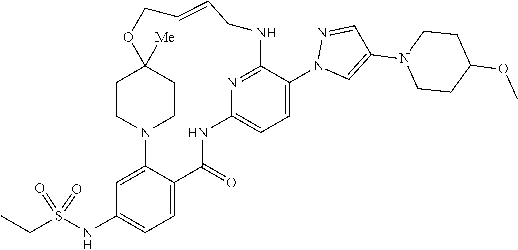 [SMILES: CCS(=O)(=O)Nc1ccc2c(c1)N1CCC(C)(CC1)OC/C=C/CNc1nc(ccc1-n1cc(N3CCC(OC)CC3)cn1)NC2=O] | 665.3 | 665.3202 |
| 608 | 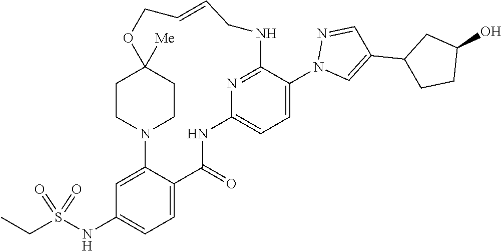 [SMILES: CCS(=O)(=O)Nc1ccc2c(c1)N1CCC(C)(CC1)OC/C=C/CNc1nc(ccc1-n1cc(C3CC[C@H](O)C3)cn1)NC2=O] | 637.1 | 401.223 |
| 609 | 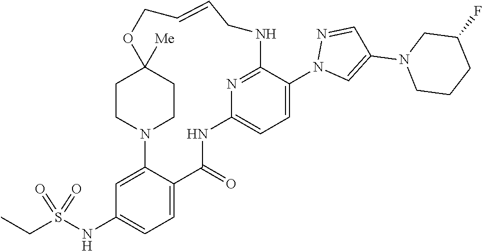 [SMILES: CCS(=O)(=O)Nc1ccc2c(c1)N1CCC(C)(CC1)OC/C=C/CNc1nc(ccc1-n1cc(N3CCC[C@@H](F)C3)cn1)NC2=O] | 653.2 | 653.3006 |
| 610 | 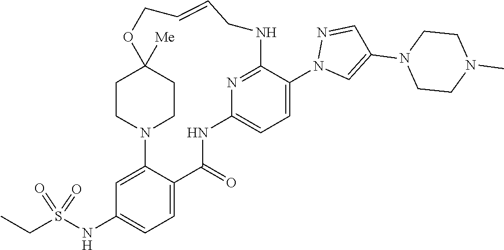 [SMILES: CCS(=O)(=O)Nc1ccc2c(c1)N1CCC(C)(CC1)OC/C=C/CNc1nc(ccc1-n1cc(N3CCN(C)CC3)cn1)NC2=O] | 650.2 | 650.3214 |
| 611 | 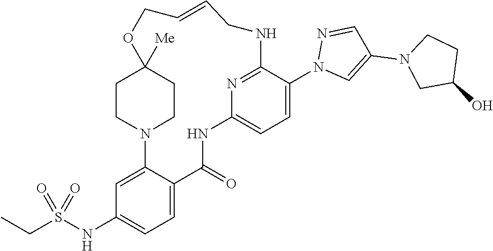 [SMILES: CCS(=O)(=O)Nc1ccc2c(c1)N1CCC(C)(CC1)OC/C=C/CNc1nc(ccc1-n1cc(N3CC[C@@H](O)C3)cn1)NC2=O] | 637.1 | 637.2872 |
| 612 | 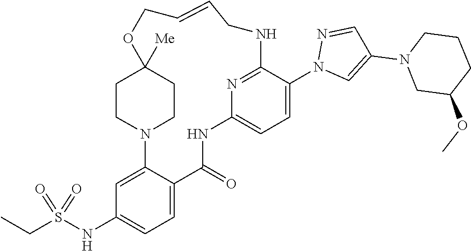 [SMILES: CCS(=O)(=O)Nc1ccc2c(c1)N1CCC(C)(CC1)OC/C=C/CNc1nc(ccc1-n1cc(N3CCC[C@@H](OC)C3)cn1)NC2=O] | 665.2 | 665.3204 |
| 613 | 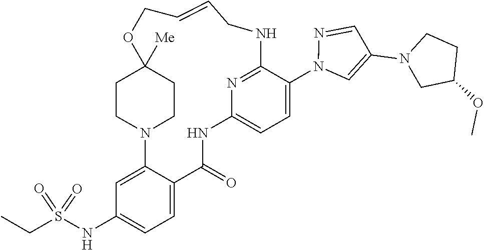 [SMILES: CCS(=O)(=O)Nc1ccc2c(c1)N1CCC(C)(CC1)OC/C=C/CNc1nc(ccc1-n1cc(N3CC[C@H](OC)C3)cn1)NC2=O] | 651.2 | 651.3045 |
| 614 | 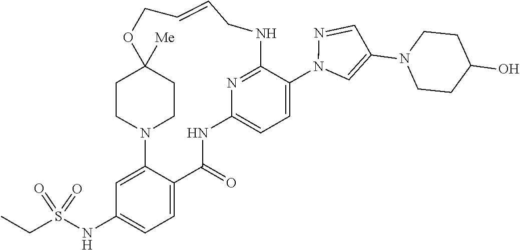 [SMILES: CCS(=O)(=O)Nc1ccc2c(c1)N1CCC(C)(CC1)OC/C=C/CNc1nc(ccc1-n1cc(N3CCC(O)CC3)cn1)NC2=O] | 651.2 | 651.3045 |
| 615 | 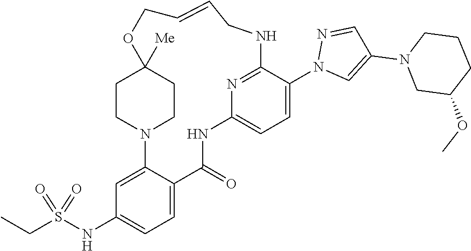 [SMILES: CCS(=O)(=O)Nc1ccc2c(c1)N1CCC(C)(CC1)OC/C=C/CNc1nc(ccc1-n1cc(N3CCC[C@H](OC)C3)cn1)NC2=O] | 665.2 | 665.3204 |
| 616 | 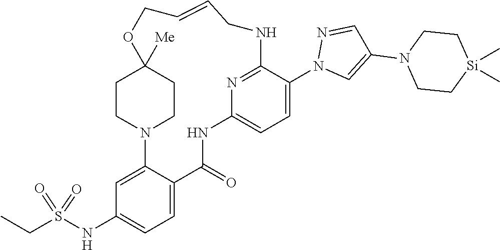 [SMILES: CCS(=O)(=O)Nc1ccc2c(c1)N1CCC(C)(CC1)OC/C=C/CNc1nc(ccc1-n1cc(N3CC[Si](C)(C)CC3)cn1)NC2=O] | 679.2 | 679.3184 |
| 617 | 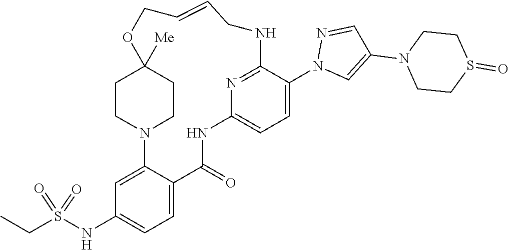 [SMILES: CCS(=O)(=O)Nc1ccc2c(c1)N1CCC(C)(CC1)OC/C=C/CNc1nc(ccc1-n1cc(N3CCS(=O)CC3)cn1)NC2=O] | 669.1 | 669.2615 |
| 618 | 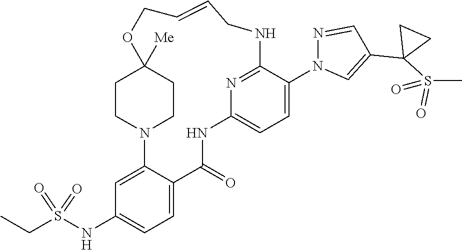 [SMILES: CCS(=O)(=O)Nc1ccc2c(c1)N1CCC(C)(CC1)OC/C=C/CNc1nc(ccc1-n1cc(C3(S(C)(=O)=O)CC3)cn1)NC2=O] | 670.3 | 670.2463 |
| 619 | 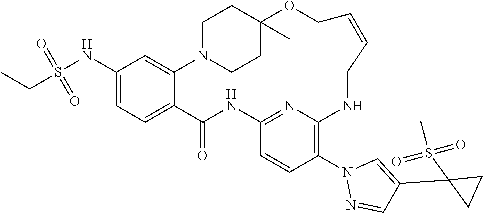 [SMILES: CCS(=O)(=O)Nc1ccc2c(c1)N1CCC(C)(CC1)OC/C=C\CNc1nc(ccc1-n1cc(C3(S(C)(=O)=O)CC3)cn1)NC2=O] | 670.3 | 670.2472 |
| 620 | 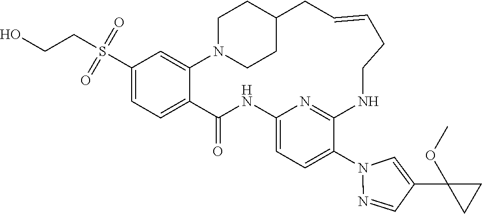 [SMILES: COC1(c2cnn(-c3ccc4nc3NCC/C=C/CC3CCN(CC3)c3cc(S(=O)(=O)CCO)ccc3C(=O)N4)c2)CC1] | | 607.2686 |
| 621 | 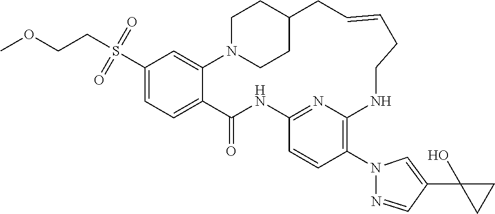 [SMILES: COCCS(=O)(=O)c1ccc2c(c1)N1CCC(C/C=C/CCNc3nc(ccc3-n3cc(C4(O)CC4)cn3)NC2=O)CC1] | 607.3 | 607.2695 |
| 622 | 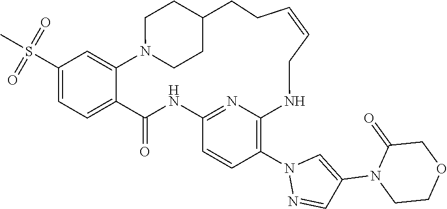 [SMILES: CS(=O)(=O)c1ccc2c(c1)N1CCC(CC/C=C\CNc3nc(ccc3-n3cc(N4CCOCC4=O)cn3)NC2=O)CC1] | 606.3 | 606.2482 |
| 623 | 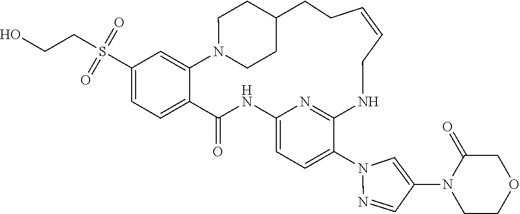 [SMILES: O=C1Nc2ccc(-n3cc(N4CCOCC4=O)cn3)c(n2)NC/C=C\CCC2CCN(CC2)c2cc(S(=O)(=O)CCO)ccc21] | 636.3 | 636.2595 |
| 624 | 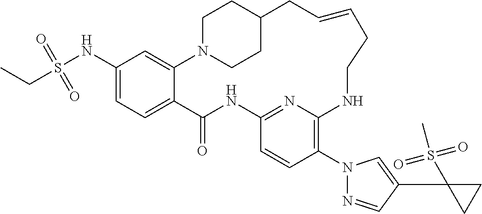 [SMILES: CCS(=O)(=O)Nc1ccc2c(c1)N1CCC(C/C=C/CCNc3nc(ccc3-n3cc(C4(S(C)(=O)=O)CC4)cn3)NC2=O)CC1] | 654.3 | 654.251 |
| 625 | 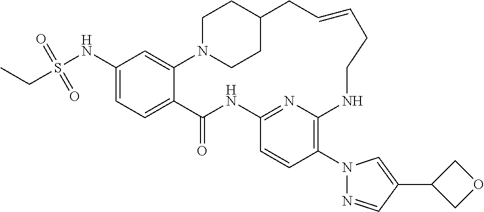 [SMILES: CCS(=O)(=O)Nc1ccc2c(c1)N1CCC(C/C=C/CCNc3nc(ccc3-n3cc(C4COC4)cn3)NC2=O)CC1] | 592.3 | 592.268 |
| 626 | 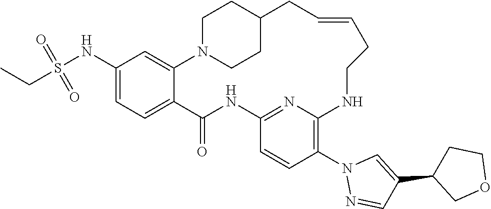 [SMILES: CCS(=O)(=O)Nc1ccc2c(c1)N1CCC(C/C=C/CCNc3nc(ccc3-n3cc([C@H]4CCOC4)cn3)NC2=O)CC1] | 606.3 | 606.284 |
| 627 | 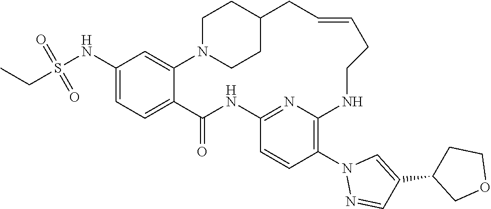 [SMILES: CCS(=O)(=O)Nc1ccc2c(c1)N1CCC(C/C=C/CCNc3nc(ccc3-n3cc([C@@H]4CCOC4)cn3)NC2=O)CC1] | 606.2 | 606.2842 |
| 628 | 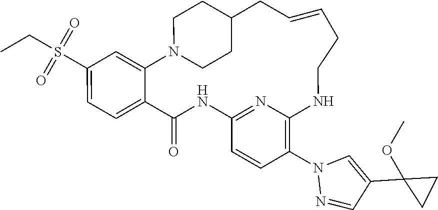 [SMILES: CCS(=O)(=O)c1ccc2c(c1)N1CCC(C/C=C/CCNc3nc(ccc3-n3cc(C4(OC)CC4)cn3)NC2=O)CC1] | 591.2 | 591.2731 |
| 629 | 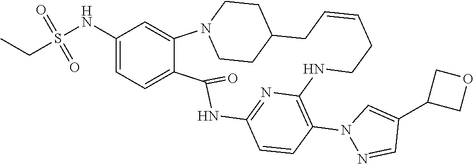 [SMILES: CCS(=O)(=O)Nc1ccc2c(c1)N1CCC(C/C=C\CCNc3nc(ccc3-n3cc(C4COC4)cn3)NC2=O)CC1] | 592.3 | 592.2683 |
| 630 | 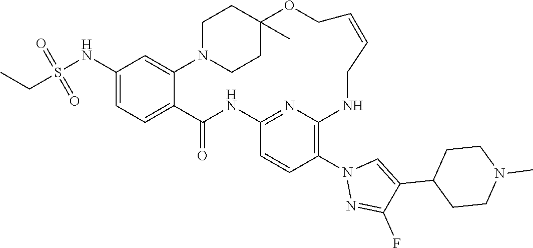 [SMILES: CCS(=O)(=O)Nc1ccc2c(c1)N1CCC(C)(CC1)OC/C=C\CNc1nc(ccc1-n1cc(C3CCN(C)CC3)c(F)n1)NC2=O] | 667.4 | 667.3161 |
| 631 | 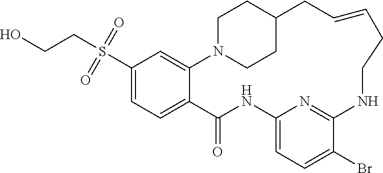 [SMILES: O=C1Nc2ccc(Br)c(n2)NCC/C=C/CC2CCN(CC2)c2cc(S(=O)(=O)CCO)ccc21] | 549.2 | 549.1141 |
| 632 | 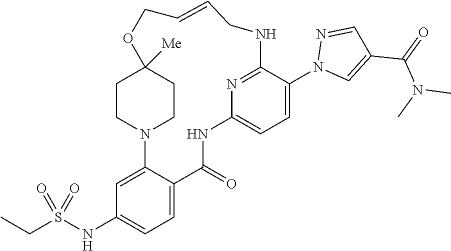 [SMILES: CCS(=O)(=O)Nc1ccc2c(c1)N1CCC(C)(CC1)OC/C=C/CNc1nc(ccc1-n1cc(C(=O)N(C)C)cn1)NC2=O] | 623.1 | 623.2741 |
| 633 | 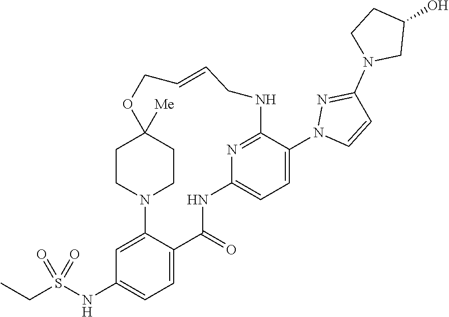 [SMILES: CCS(=O)(=O)Nc1ccc2c(c1)N1CCC(C)(CC1)OC/C=C/CNc1nc(ccc1-n1ccc(N3CC[C@H](O)C3)n1)NC2=O] | 637.3 | 637.2904 |
| 634 | 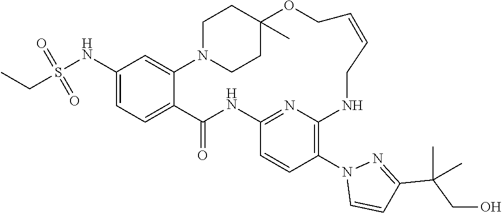 [SMILES: CCS(=O)(=O)Nc1ccc2c(c1)N1CCC(C)(CC1)OC/C=C\CNc1nc(ccc1-n1ccc(C(C)(C)CO)n1)NC2=O] | 624.2 | 624.2958 |
| 635 | 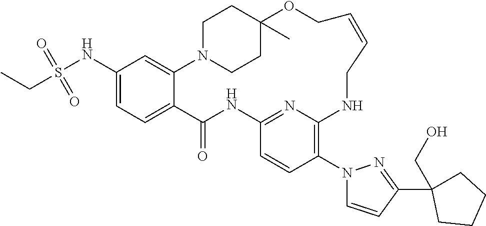 [SMILES: CCS(=O)(=O)Nc1ccc2c(c1)N1CCC(C)(CC1)OC/C=C\CNc1nc(ccc1-n1ccc(C3(CO)CCCC3)n1)NC2=O] | 650.2 | 650.3115 |
| 636 | 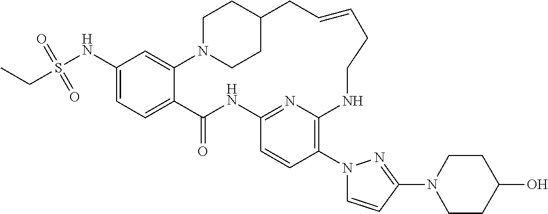 [SMILES: CCS(=O)(=O)Nc1ccc2c(c1)N1CCC(C/C=C/CCNc3nc(ccc3-n3ccc(N4CCC(O)CC4)n3)NC2=O)CC1] | 635.2 | 635.3101 |
| 637 | 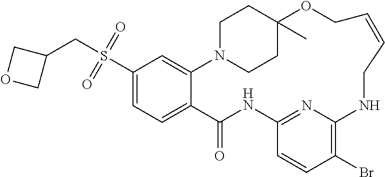 [SMILES: CC12CCN(CC1)c1cc(S(=O)(=O)CC3COC3)ccc1C(=O)Nc1ccc(Br)c(n1)NC/C=C\CO2] | 593.0 | 591.1265 |
| 638 | 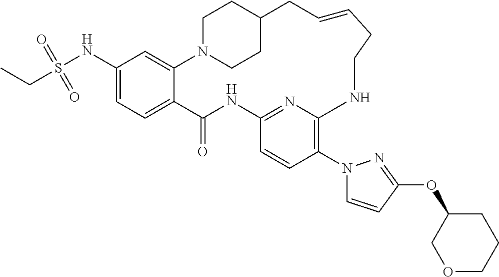 [SMILES: CCS(=O)(=O)Nc1ccc2c(c1)N1CCC(C/C=C/CCNc3nc(ccc3-n3ccc(O[C@H]4CCCOC4)n3)NC2=O)CC1] | 636.2 | 636.2932 |
| 639 | 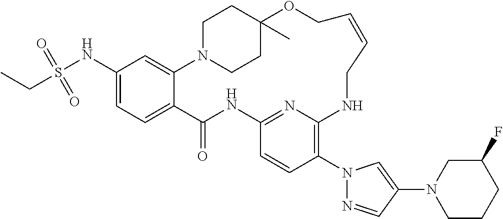 [SMILES: CCS(=O)(=O)Nc1ccc2c(c1)N1CCC(C)(CC1)OC/C=C\CNc1nc(ccc1-n1cc(N3CCC[C@H](F)C3)cn1)NC2=O] | 653.2 | 653.3008 |
| 640 | 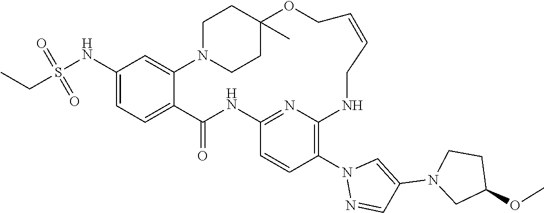 [SMILES: CCS(=O)(=O)Nc1ccc2c(c1)N1CCC(C)(CC1)OC/C=C\CNc1nc(ccc1-n1cc(N3CC[C@@H](OC)C3)cn1)NC2=O] | 651.2 | 651.3053 |
| 641 | 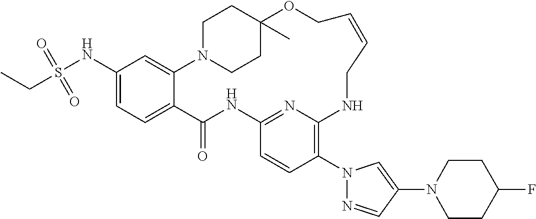 [SMILES: CCS(=O)(=O)Nc1ccc2c(c1)N1CCC(C)(CC1)OC/C=C\CNc1nc(ccc1-n1cc(N3CCC(F)CC3)cn1)NC2=O] | 653.3 | 653.3007 |
| 642 | 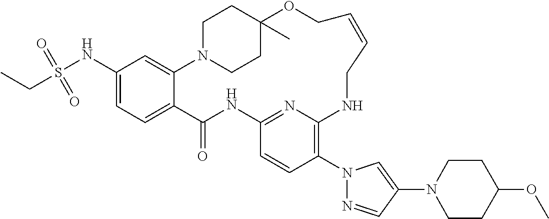 [SMILES: CCS(=O)(=O)Nc1ccc2c(c1)N1CCC(C)(CC1)OC/C=C\CNc1nc(ccc1-n1cc(N3CCC(OC)CC3)cn1)NC2=O] | 665.2 | 665.3204 |
| 643 | 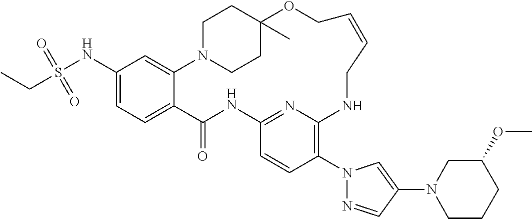 [SMILES: CCS(=O)(=O)Nc1ccc2c(c1)N1CCC(C)(CC1)OC/C=C\CNc1nc(ccc1-n1cc(N3CCC[C@@H](OC)C3)cn1)NC2=O] | 665.2 | 665.321 |
| 644 | 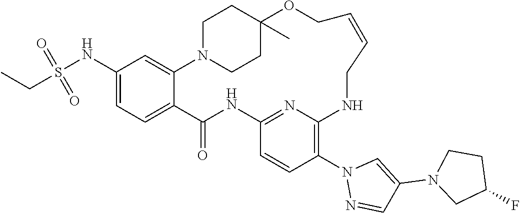 [SMILES: CCS(=O)(=O)Nc1ccc2c(c1)N1CCC(C)(CC1)OC/C=C\CNc1nc(ccc1-n1cc(N3CC[C@H](F)C3)cn1)NC2=O] | 639.2 | 639.2849 |
| 645 | 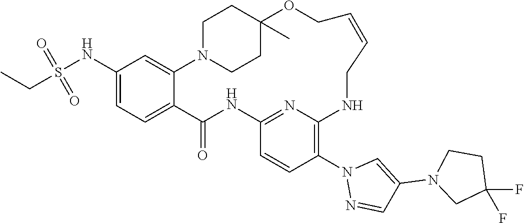 [SMILES: CCS(=O)(=O)Nc1ccc2c(c1)N1CCC(C)(CC1)OC/C=C\CNc1nc(ccc1-n1cc(N3CCC(F)(F)C3)cn1)NC2=O] | 657.2 | 657.2776 |
| 646 | 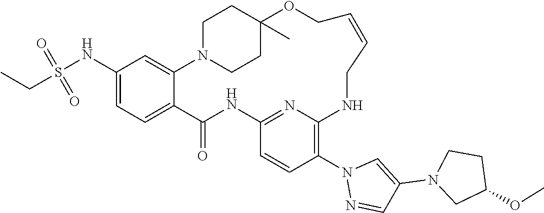 [SMILES: CCS(=O)(=O)Nc1ccc2c(c1)N1CCC(C)(CC1)OC/C=C\CNc1nc(ccc1-n1cc(N3CC[C@H](OC)C3)cn1)NC2=O] | 651.2 | 651.305 |
| 647 | 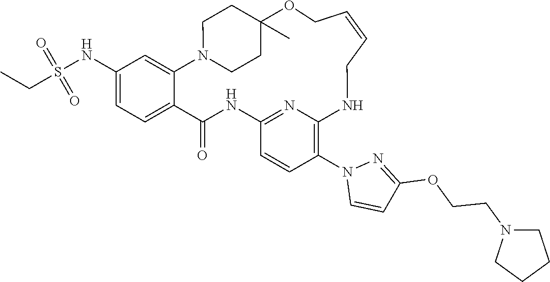 [SMILES: CCS(=O)(=O)Nc1ccc2c(c1)N1CCC(C)(CC1)OC/C=C\CNc1nc(ccc1-n1ccc(OCCN3CCCC3)n1)NC2=O] | 665.2 | 665.3221 |
| 648 | 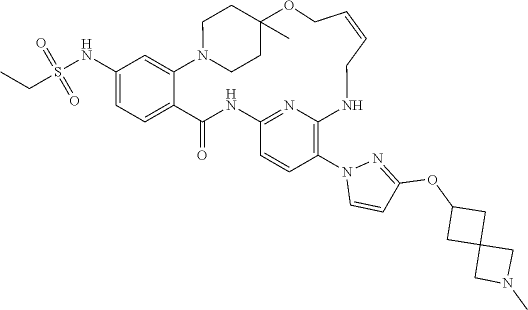 [SMILES: CCS(=O)(=O)Nc1ccc2c(c1)N1CCC(C)(CC1)OC/C=C\CNc1nc(ccc1-n1ccc(OC3CC4(C3)CN(C)C4)n1)NC2=O] | 677.3 | 677.3228 |
| 649 | 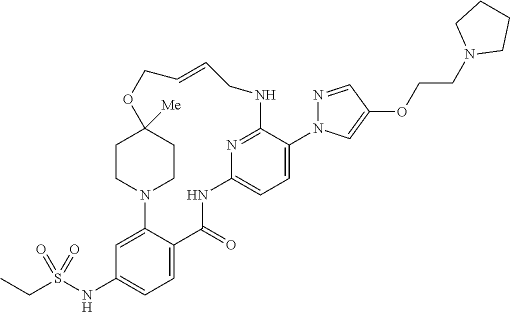 [SMILES: CCS(=O)(=O)Nc1ccc2c(c1)N1CCC(C)(CC1)OC/C=C/CNc1nc(ccc1-n1cc(OCCN3CCCC3)cn1)NC2=O] | 665.3 | 665.3217 |
| 650 | 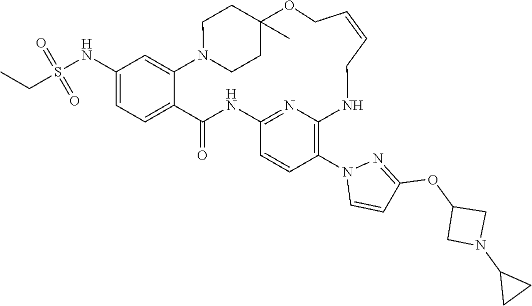 [SMILES: CCS(=O)(=O)Nc1ccc2c(c1)N1CCC(C)(CC1)OC/C=C\CNc1nc(ccc1-n1ccc(OC3CN(C4CC4)C3)n1)NC2=O] | 663.3 | 663.3063 |
| 651 | 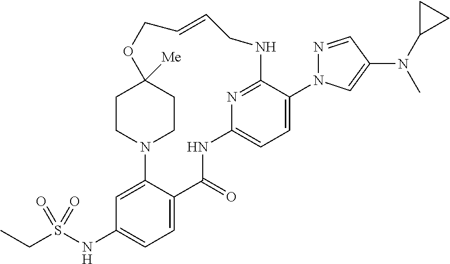 [SMILES: CCS(=O)(=O)Nc1ccc2c(c1)N1CCC(C)(CC1)OC/C=C/CNc1nc(ccc1-n1cc(N(C)C3CC3)cn1)NC2=O] | 635.2 | 635.3091 |
| 652 | 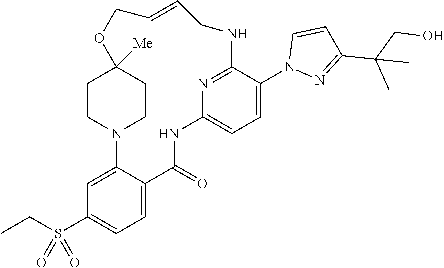 [SMILES: CCS(=O)(=O)c1ccc2c(c1)N1CCC(C)(CC1)OC/C=C/CNc1nc(ccc1-n1ccc(C(C)(C)CO)n1)NC2=O] | 609.2 | 609.2847 |
| 653 | 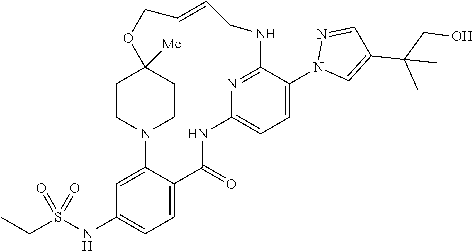 [SMILES: CCS(=O)(=O)Nc1ccc2c(c1)N1CCC(C)(CC1)OC/C=C/CNc1nc(ccc1-n1cc(C(C)(C)CO)cn1)NC2=O] | 624.2 | 624.2952 |
| 654 | 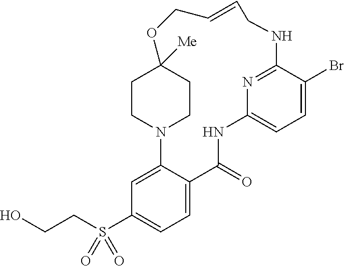 [SMILES: CC12CCN(CC1)c1cc(S(=O)(=O)CCO)ccc1C(=O)Nc1ccc(Br)c(n1)NC/C=C/CO2] | 656.1 | |
| 655 | 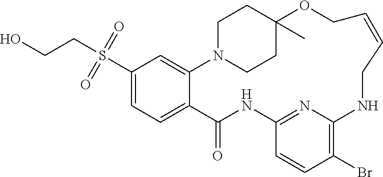 [SMILES: CC12CCN(CC1)c1cc(S(=O)(=O)CCO)ccc1C(=O)Nc1ccc(Br)c(n1)NC/C=C\CO2] | 565.2 | |
| 656 | 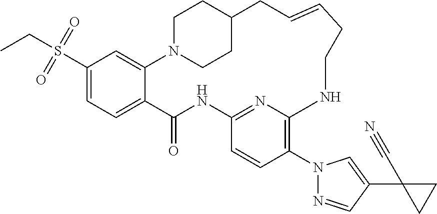 [SMILES: CCS(=O)(=O)c1ccc2c(c1)N1CCC(C/C=C/CCNc3nc(ccc3-n3cc(C4(C#N)CC4)cn3)NC2=O)CC1] | 586.3 | 586.2589 |
| 657 | 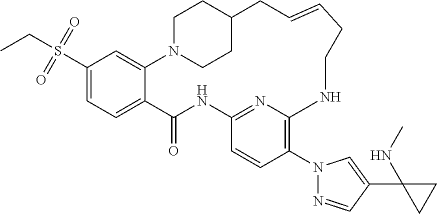 [SMILES: CCS(=O)(=O)c1ccc2c(c1)N1CCC(C/C=C/CCNc3nc(ccc3-n3cc(C4(NC)CC4)cn3)NC2=O)CC1] | 590.4 | 590.2893 |
| 658 | 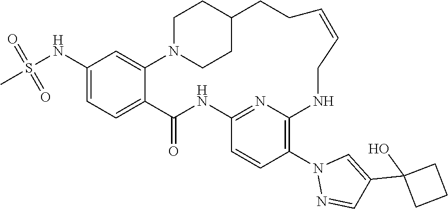 [SMILES: CS(=O)(=O)Nc1ccc2c(c1)N1CCC(CC/C=C\CNc3nc(ccc3-n3cc(C4(O)CCC4)cn3)NC2=O)CC1] | 578.4 | |
| 659 | 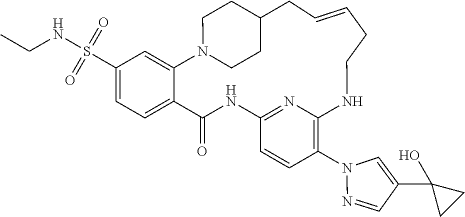 [SMILES: CCNS(=O)(=O)c1ccc2c(c1)N1CCC(C/C=C/CCNc3nc(ccc3-n3cc(C4(O)CC4)cn3)NC2=O)CC1] | 609.3 | 609.2471 |
| 660 | 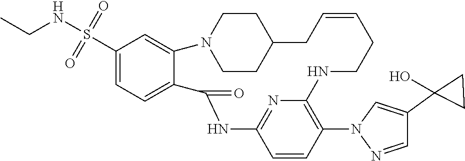 [SMILES: CCNS(=O)(=O)c1ccc2c(c1)N1CCC(C/C=C\CCNc3nc(ccc3-n3cc(C4(O)CC4)cn3)NC2=O)CC1] | 592.1 | |
| 661 | 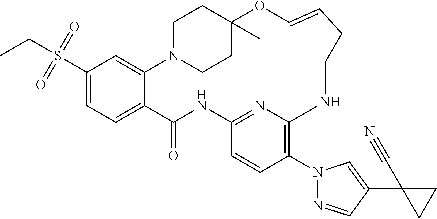 [SMILES: CCS(=O)(=O)c1ccc2c(c1)N1CCC(C)(CC1)O/C=C/CCNc1nc(ccc1-n1cc(C3(C#N)CC3)cn1)NC2=O] | 602.2 | |
| 662 | 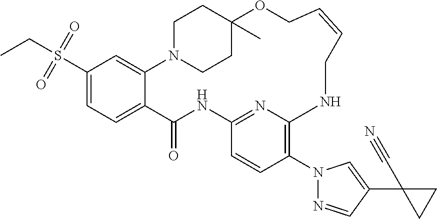 [SMILES: CCS(=O)(=O)c1ccc2c(c1)N1CCC(C)(CC1)OC/C=C\CNc1nc(ccc1-n1cc(C3(C#N)CC3)cn1)NC2=O] | 602.2 | |
| 663 | 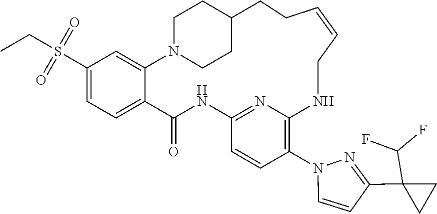 [SMILES: CCS(=O)(=O)c1ccc2c(c1)N1CCC(CC/C=C\CNc3nc(ccc3-n3ccc(C4(C(F)F)CC4)n3)NC2=O)CC1] | 611.4 | |
| 664 | 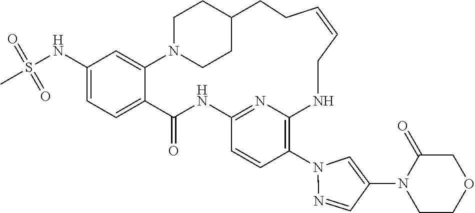 [SMILES: CS(=O)(=O)Nc1ccc2c(c1)N1CCC(CC/C=C\CNc3nc(ccc3-n3cc(N4CCOCC4=O)cn3)NC2=O)CC1] | 621.3 | |
| 665 | 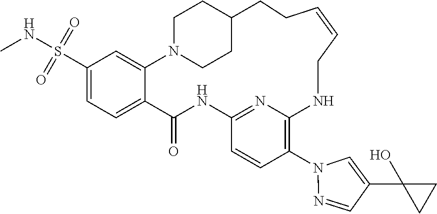 [SMILES: CNS(=O)(=O)c1ccc2c(c1)N1CCC(CC/C=C\CNc3nc(ccc3-n3cc(C4(O)CC4)cn3)NC2=O)CC1] | 578.4 | |
| 666 | 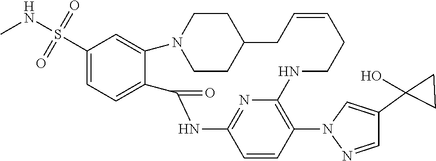 [SMILES: CNS(=O)(=O)c1ccc2c(c1)N1CCC(C/C=C\CCNc3nc(ccc3-n3cc(C4(O)CC4)cn3)NC2=O)CC1] | 578.3 | |
| 667 | 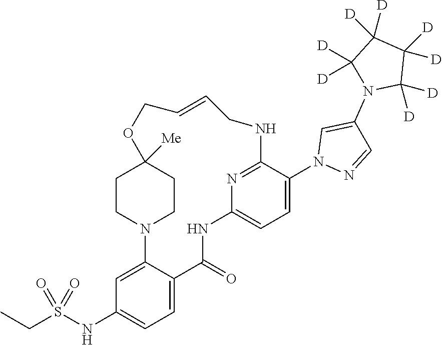 [SMILES: [2H]C1([2H])N(c2cnn(-c3ccc4nc3NC/C=C/COC3(C)CCN(CC3)c3cc(NS(=O)(=O)CC)ccc3C(=O)N4)c2)C([2H])([2H])C([2H])([2H])C1([2H])[2H]] | 629.3 | |
| 668 | 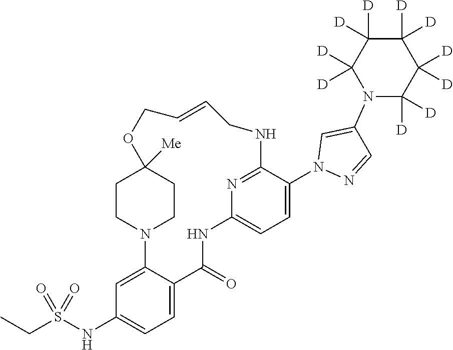 [SMILES: [2H]C1([2H])N(c2cnn(-c3ccc4nc3NC/C=C/COC3(C)CCN(CC3)c3cc(NS(=O)(=O)CC)ccc3C(=O)N4)c2)C([2H])([2H])C([2H])([2H])C([2H])([2H])C1([2H])[2H]] | 645.3 | |
| 669 | 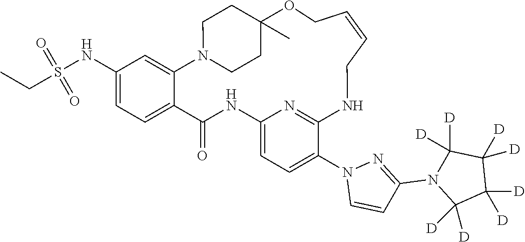 [SMILES: [2H]C1([2H])N(c2ccn(-c3ccc4nc3NC/C=C\COC3(C)CCN(CC3)c3cc(NS(=O)(=O)CC)ccc3C(=O)N4)n2)C([2H])([2H])C([2H])([2H])C1([2H])[2H]] | 629.3 | |
| 670 | 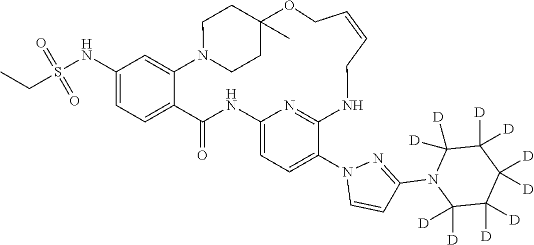 [SMILES: [2H]C1([2H])N(c2ccn(-c3ccc4nc3NC/C=C\COC3(C)CCN(CC3)c3cc(NS(=O)(=O)CC)ccc3C(=O)N4)n2)C([2H])([2H])C([2H])([2H])C([2H])([2H])C1([2H])[2H]] | 645.3 | |
| 671 | 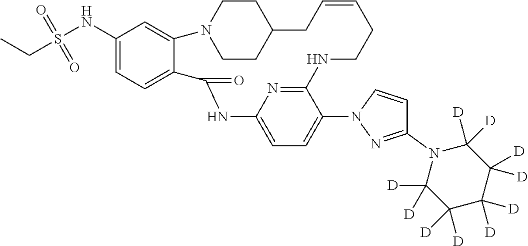 [SMILES: [2H]C1([2H])N(c2ccn(-c3ccc4nc3NCC/C=C\CC3CCN(CC3)c3cc(NS(=O)(=O)CC)ccc3C(=O)N4)n2)C([2H])([2H])C([2H])([2H])C([2H])([2H])C1([2H])[2H]] | 629.2 | |
| 672 | 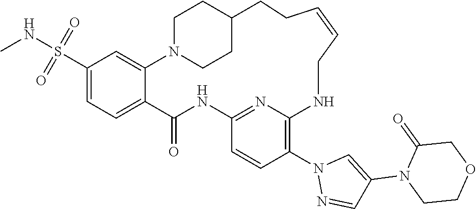 [SMILES: CNS(=O)(=O)c1ccc2c(c1)N1CCC(CC/C=C\CNc3nc(ccc3-n3cc(N4CCOCC4=O)cn3)NC2=O)CC1] | 621.4 | |
| 673 | 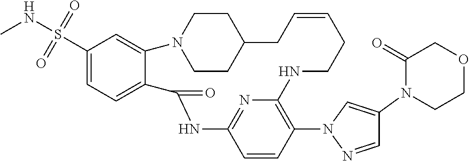 [SMILES: CNS(=O)(=O)c1ccc2c(c1)N1CCC(C/C=C\CCNc3nc(ccc3-n3cc(N4CCOCC4=O)cn3)NC2=O)CC1] | 621.4 | |
| 674 | 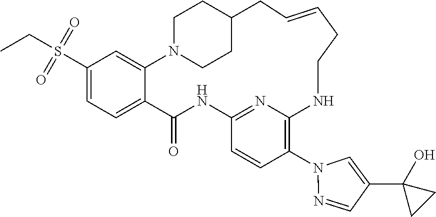 [SMILES: CCS(=O)(=O)c1ccc2c(c1)N1CCC(C/C=C/CCNc3nc(ccc3-n3cc(C4(O)CC4)cn3)NC2=O)CC1] | 577.3 | |
| 675 | 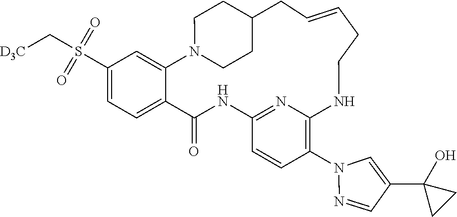 [SMILES: [2H]C([2H])([2H])CS(=O)(=O)c1ccc2c(c1)N1CCC(C/C=C/CCNc3nc(ccc3-n3cc(C4(O)CC4)cn3)NC2=O)CC1] | 580.3 | |
| 676 | 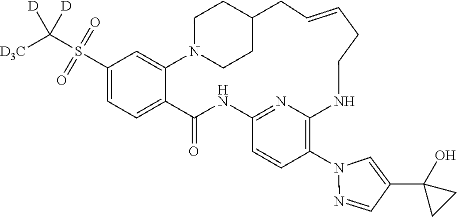 [SMILES: [2H]C([2H])([2H])C([2H])([2H])S(=O)(=O)c1ccc2c(c1)N1CCC(C/C=C/CCNc3nc(ccc3-n3cc(C4(O)CC4)cn3)NC2=O)CC1] | 582.3 | |

Characterization data of compounds in Table 1:

| Cpd | |
|---|---|
| No. | ¹H NMR (ppm) |
| 1 | ¹H NMR (400 MHz, DMSO-d6) δ 10.89 (s, 1H), 10.20 (s, 1H), 8.17 (d, J = 2.5 Hz, 1H), 7.89 (dd, |
| | J = 8.5, 1.4 Hz, 1H), 7.81 (d, J = 1.9 Hz, 1H), 7.74-7.55 (m, 2H), 7.18-6.89 (m, 3H), 6.55 (t, J = |
| | 2.2 Hz, 1H), 5.55 (ddq, J = 28.2, 15.4, 7.2, 6.5 Hz, 2H), 3.67 (p, J = 3.3 Hz, 2H), 3.28-3.11 |
| | (m, 4H), 2.68 (t, J = 11.5 Hz, 2H), 2.17 (d, J = 5.1 Hz, 2H), 2.06 (dd, J = 7.6, 3.8 Hz, 2H), 1.92- |
| | 1.42 (m, 5H), 1.21 (td, J = 7.4, 1.6 Hz, 3H) |
| 2 | ¹H NMR (400 MHz, DMSO-d₆) δ 12.52 (s, 1 H) 10.26 (s, 1 H) 8.17 (d, J = 2.5 Hz, 1 H) 8.12 (d, |
| | J = 8.5 Hz, 1 H) 7.81 (d, J = 1.8 Hz, 1 H) 7.66 (d, J = 8.0 Hz, 1 H) 7.36 (d, J = 8.3 Hz, 1 H) 7.25 (d, |
| | J = 2.0 Hz, 1 H) 7.09-7.21 (m, 2 H) 6.55 (t, J = 1.9 Hz, 1 H) 5.42-5.57 (m, 2 H) 3.39-3.54 (m, 2 |
| | H) 3.22 (q, J = 7.3 Hz, 2 H) 3.12 (br d, J = 10.8 Hz, 2 H) 2.78 (br t, J = 11.0 Hz, 2 H) 2.22-2.35 (m, |
| | 2 H) 2.18 (br t, J = 6.9 Hz, 2 H) 1.86-2.03 (m, 2 H) 1.67-1.86 (m, 3 H) 1.21 (t, J = 7.4 Hz, 3 H) |
| 5 | ¹H NMR (400 MHz, DMSO-d6) δ 10.89 (s, 1H), 10.20 (s, 1H), 8.06 (s, 1H), 7.89 (d, J = 8.4 Hz, |
| | 1H), 7.64 (d, J = 8.4 Hz, 1H), 7.59 (d, J = 8.4 Hz, 1H), 7.09 (s, 1H), 7.04-6.99 (m, 2H), 6.47 (s, |
| | 1H), 5.63-5.49 (m, 2H), 5.19-5.16 (m, 1H), 4.54-4.52 (m, 2H), 3.68 (s, 2H), 3.22-3.18 (m, 4H), |
| | 2.71-2.64 (m, 2H), 2.20-2.17 (m, 2H), 2.07-2.04 (m, 2H), 1.83-1.76 (m, 2H), 1.73-1.65 (m, 1H), |
| | 1.54-1.49 (m, 2H), 1.21 (t, J = 7.2 Hz, 3H). |
| 6 | ¹H NMR (400 MHz, DMSO-d6) δ 12.49 (s, 1H), 10.25 (s, 1H), 8.12-8.07 (m, 2H), 7.63 (d, J = |
| | 8.4 Hz, 1H), 7.34 (d, J = 8.4 Hz, 1H), 7.27-7.23 (m, 2H), 7.15-7.12 (m, 1H), 6.47 (s, 1H), 5.52- |
| | 5.49 (m, 2H), 5.20-5.12 (m, 1H), 4.54-4.52 (m, 2H), 3.48-3.45 (m, 2H), 3.22-3.20 (m, 2H), 3.13- |
| | 3.10 (m, 2H), 2.81-2.73 (m, 2H), 2.31-2.22 (m, 2H), 2.21-2.16 (m, 2H), 1.99-1.87 (m, 2H), 1.86- |
| | 1.79 (m, 2H), 1.78-1.67 (m, 1H), 1.23-1.21 (m, 3H). |
| 8 | ¹H NMR (400 MHz, DMSO-d₆) δ 10.95 (s, 1 H) 10.21 (s, 1 H) 8.31 (d, J = 2.5 Hz, 1 H) 7.89 (d, |
| | J = 8.5 Hz, 1 H) 7.58-7.69 (m, 2 H) 7.10 (d, J = 2.0 Hz, 1 H) 7.00-7.07 (m, 2 H) 6.39 (t, J = 6.5 |
| | Hz, 1 H) 5.46-5.66 (m, 2 H) 3.56-3.69 (m, 2 H) 3.34 (s, 3 H) 3.14-3.27 (m, 4 H) 2.69 (br t, |
| | J = 11.4 Hz, 2 H) 2.11-2.21 (m, 2 H) 2.07 (br dd, J = 6.4, 3.6 Hz, 2 H) 1.73-1.88 (m, 2 H) 1.60- |
| | 1.73 (m, 1 H) 1.54 (br d, J = 11.3 Hz, 2 H) 1.21 (t, J = 7.4 Hz, 3 H) |
| 9 | ¹H NMR (400 MHz, DMSO-d₆) δ 12.63 (s, 1 H) 10.28 (br s, 1 H) 8.30 (d, J = 2.5 Hz, 1 H) 8.11 (d, |
| | J = 8.8 Hz, 1 H) 7.66 (d, J = 8.3 Hz, 1 H) 7.40 (d, J = 8.3 Hz, 1 H) 7.25 (d, J = 2.3 Hz, 1 H) 7.15 (dd, |
| | J = 8.8, 2.3 Hz, 1 H) 7.06 (d, J = 2.5 Hz, 1 H) 6.65 (t, J = 6.4 Hz, 1 H) 5.44-5.57 (m, 2 H) 3.38- |
| | 3.48 (m, 2 H) 3.35 (s, 3 H) 3.22 (q, J = 7.4 Hz, 2 H) 3.12 (br d, J = 11.3 Hz, 2 H) 2.78 (br t, J = 11.3 |
| | Hz, 2 H) 2.22-2.30 (m, 2 H) 2.18 (br t, J = 6.9 Hz, 2 H) 1.68-1.98 (m, 5 H) 1.21 (t, J = 7.3 Hz, 3 |
| | H) |
| 11 | 1H NMR (400 MHz, DMSO-d6) δ 10.95 (s, 1H), 10.21 (s, 1H), 8.33 (d, J = 2.4 Hz, 1H), 7.89 (d, |
| | J = 8.4 Hz, 1H), 7.65 (d, J = 8.4 Hz, 1H), 7.59 (d, J = 8.4 Hz, 1H), 7.25 (d, J = 2.8 Hz, 1H), 7.10 |
| | (d, J = 2.4 Hz, 1H), 7.04-7.01 (m, 1H), 6.45-6.41 (m, 1H), 5.57-5.51 (m, 2H), 3.65-3.56 |
| | (m, 4H), 3.27-3.16 (m, 4H), 2.68 (s, 2H), 2.21-2.02 (m, 2H), 1.87-1.74 (m, 2H), 1.73-1.60 |
| | (m, 1H), 1.58-1.50 (m, 2H), 1.23-1.19 (m, 3H). |
| 12 | 1H NMR (400 MHz, DMSO-d6) δ 12.63 (s, 1H), 10.27 (s, 1H), 8.33 (d, J = 2.4 Hz, 1H), 8.12 (d, |
| | J = 8.4 Hz, 1H), 7.64 (d, J = 8.4 Hz, 1H), 7.38 (d, J = 8.4 Hz, 1H), 7.25 (d, J = 2.4 Hz, 2H), 7.16- |
| | 7.14 (m, 1H), 6.71-6.68 (m, 1H), 5.52-5.49 (m, 2H), 3.42-3.38 (m, 2H), 3.22-3.19 (m, 2H), |
| | 3.13-3.10 (m, 2H), 2.81-2.75 (m, 2H), 2.33-2.23 (m, 2H), 2.20-2.16 (m, 2H), 1.92-1.74 |
| | (m, 5H), 1.23-1.19 (m, 3H). |
| 14 | 1H NMR (400 MHz, DMSO-d6) δ 10.86 (s, 1H), 10.19 (s, 1H), 7.93 (d, J = 2.6 Hz, 1H), 7.88 (d, |
| | J = 8.5 Hz, 1H), 7.62 (d, J = 8.3 Hz, 1H), 7.50 (d, J = 8.3 Hz, 1H), 7.09 (d, J = 2.1 Hz, 1H), 7.02 |
| | (dd, J = 8.6, 2.0 Hz, 1H), 6.78 (t, J = 6.8 Hz, 1H), 5.99 (d, J = 2.5 Hz, 1H), 5.66-5.47 (m, 2H), |
| | 3.87 (s, 3H), 3.69-3.62 (m, 2H), 3.26-3.15 (m, 4H), 2.67 (t, J = 11.5 Hz, 2H), 2.18 (q, J = 5.9 |
| | Hz, 2H), 2.06 (dd, J = 7.4, 3.7 Hz, 2H), 1.87-1.74 (m, 2H), 1.67 (d, J = 12.3 Hz, 1H), 1.53 (d, J = |
| | 11.8 Hz, 2H), 1.21 (t, J = 7.3 Hz, 3H). |
| 15 | 1H NMR (400 MHz, DMSO-d6) δ 12.50 (s, 1H), 10.24 (s, 1H), 8.10 (d, J = 8.7 Hz, 1H), 7.91 (t, J = |
| | 2.2 Hz, 1H), 7.53 (d, J = 8.3 Hz, 1H), 7.30 (d, J = 8.2 Hz, 1H), 7.23 (d, J = 2.2 Hz, 1H), 7.13 |
| | (dd, J = 8.7, 2.1 Hz, 1H), 6.98 (t, J = 6.5 Hz, 1H), 5.98 (d, J = 2.5 Hz, 1H), 5.54-5.43 (m, 2H), |
| | 3.86 (s, 2H), 3.45 (s, 4H), 3.26-3.14 (m, 2H), 3.10 (d, J = 11.2 Hz, 2H), 2.77 (td, J = 11.8, 2.5 |
| | Hz, 2H), 2.27 (dt, J = 11.9, 6.1 Hz, 2H), 2.17 (t, J = 7.2 Hz, 2H), 1.97-1.76 (m, 3H), 1.21 (t, J = |
| | 7.3 Hz, 3H). |
| 17 | ¹H NMR (400 MHz, DMSO) δ 10.94 (s, 1H), 10.23-10.19 (m, 1H), 8.91 (s, 1H), 8.38 (s, 1H), |
| | 7.88 (s, 1H), 7.68-7.51 (m, 2H), 7.13-6.99 (m, 2H), 6.42 (t, J = 6.6 Hz, 1H), 5.65-5.45 (m, |
| | 2H), 3.60 (s, 2H), 3.24-3.17 (m, 4H), 2.68 (t, J = 11.6 Hz, 2H), 2.17-2.12 (m, 2H), 2.08-2.02 |
| | (m, 2H), 1.78 (t, J = 12.0 Hz, 2H), 1.68-1.63 (m, 1H), 1.54 (d, J = 12.3 Hz, 2H), 1.21 (t, J = 7.3 |
| | Hz, 3H). |
| 18 | ¹H NMR (400 MHz, DMSO) δ 12.64 (s, 1H), 8.89 (s, 1H), 8.36 (s, 1H), 8.10 (d, J = 1.4 Hz, 1H), |
| | 7.61 (d, J = 8.2 Hz, 1H), 7.36 (d, J = 8.3 Hz, 1H), 7.23 (q, J = 2.0 Hz, 1H), 7.13 (dq, J = 8.8, 1.9 |
| | Hz, 1H), 6.65 (t, J = 6.4 Hz, 2H), 5.49 (t, J = 5.8 Hz, 2H), 3.25-3.15 (m, 3H), 3.10 (d, J = 11.2 |
| | Hz, 3H), 2.83-2.73 (m, 1H), δ 2.27-2.20 (m, 2H), 2.17 (t, J = 7.0 Hz, 3H), 1.96-1.84 (m, |
| | 2H), 1.83-1.76 (m, 3H)., 1.21 (t, J = 7.2 Hz, 3H). |
| 20 | ¹H NMR (400 MHz, DMSO-d₆) δ 10.92 (s, 1H), 10.21 (s, 1H), 8.48 (s, 1H), 8.14 (s, 1H), 7.89 (d, |
| | J = 8.5 Hz, 1H), 7.74-7.51 (m, 3H), 7.21-6.95 (m, 3H), 6.66 (d, J = 7.0 Hz, 1H), 5.78-5.32 |
| | (m, 2H), 3.63 (s, 2H), 3.21 (t, J = 8.1 Hz, 4H), 2.74-2.61 (m, 2H), 2.39-2.30 (m, 2H), 2.16 (s, |
| | 1H), 2.06 (s, 1H), 1.83-1.15 (m, 5H), 1.21 (t, J = 7.3 Hz, 3H). |
| 21 | ¹H NMR (400 MHz, DMSO-d₆) δ 12.58 (s, 1H), 10.27 (s, 1H), 8.48 (s, 1H), 8.23-8.00 (m, 2H), |
| | 7.75-7.48 (m, 2H), 7.37 (d, J = 8.2 Hz, 1H), 7.34-7.06 (m, 3H), 6.88 (d, J = 6.5 Hz, 1H), 5.49 |
| | (d, J = 5.9 Hz, 2H), 3.55-3.35 (m, 2H), 3.21 (t, J = 7.3 Hz, 2H), 3.15-3.09 (m, 2H), 2.78 (t, J = |
| | 11.4 Hz, 2H), 2.36-2.21 (m, 2H), 2.17 (d, J = 6.9 Hz, 2H), 1.91 (t, J = 11.7 Hz, 2H), 1.82-1.71 |
| | (m, 3H), 1.21 (t, J = 7.3 Hz, 3H). |
| 23 | ¹H NMR (400 MHz, DMSO) δ 10.89 (s, 1H), 10.20 (s, 1H), 8.05 (s, 1H), 7.89 (d, J = 8.5 Hz, |
| | 1H), 7.74 (s, 1H), 7.68-7.57 (m, 2H), 7.10 (d, J = 2.1 Hz, 1H), 7.02 (t, J = 8.0 Hz, 2H), 5.65- |
| | 5.44 (m, 1H), 5.00 (t, J = 5.4 Hz, 1H), 4.47 (d, J = 5.4 Hz, 2H), 3.67 (s, 2H), 3.22 (q, J = 7.8 Hz, |
| | 3H), 2.71-2.64 (m, 2H), 2.32 (d, J = 11.5 Hz, 1H), 2.17 (s, 2H), 2.07-2.02 (m, 2H), 1.80 (t, J = |
| | 11.8 Hz, 2H), 1.68-1.64 (m, 2H), 1.53 (d, J = 12.4 Hz, 2H), 1.21 (t, J = 7.3 Hz, 3H). |
| 24 | ¹H NMR (400 MHz, DMSO) δ 12.51 (s, 1H), 10.26 (s, 1H), 8.12 (d, J = 8.7 Hz, 1H), 8.05 (s, |
| | 1H), 7.73 (s, 1H), 7.63 (d, J = 8.2 Hz, 1H), 7.35 (d, J = 8.2 Hz, 1H), 7.23 (dd, J = 8.0, 4.4 Hz, |
| | 2H), 7.15 (d, J = 8.8 Hz, 1H), 5.51 (t, J = 5.6 Hz, 1H), 5.00 (t, J = 5.4 Hz, 1H), 4.46 (d, J = 5.2 |
| | Hz, 2H), 3.50-3.41 (m, 2H), 3.22 (q, J = 7.2 Hz, 2H), 3.12 (d, J = 11.2 Hz, 2H), 2.78 (t, J = 11.4 |
| | Hz, 2H), 2.68 (s, 1H), 2.34-2.24 (m, 3H), 2.18 (s, 1H), 1.96-1.88 (m, 2H), 1.84-1.74 (m, |
| | 2H), 1.21 (t, J = 7.2 Hz, 4H). |
| 25 | 1H NMR (400 MHz, DMSO-d6) δ 10.80 (s, 1H), 10.21 (s, 1H), 8.17 (s, 1H), 7.86 (d, J = 8.4 Hz, |
| | 1H), 7.80 (s, 1H), 7.63 (s, 2H), 7.14 (s, 1H), 7.02-7.00 (m, 2H), 6.55 (s, 1H), 5.74-5.66 (m, 1H), |
| | 5.54-5.47 (m, 1H), 3.71 (s, 2H), 3.25-3.17 (m, 2H), 3.04-2.90 (m, 2H), 2.92-2.84 (m, 2H), 2.19 (s, |
| | 2H), 2.08-1.98 (m, 2H), 1.96-1.89 (m, 2H), 1.25-1.12 (m, 5H), 1.00 (s, 3H). |
| 26 | ¹H NMR (400 MHz, DMSO-d₆) δ 12.49 (s, 1 H) 10.25 (br s, 1 H) 8.05-8.26 (m, 2 H) 7.80 (d, |
| | J = 1.8 Hz, 1 H) 7.65 (d, J = 8.3 Hz, 1 H) 7.26-7.39 (m, 2 H) 7.11-7.24 (m, 2 H) 6.48-6.61 (m, 1 |
| | H) 5.46-5.67 (m, 2 H) 3.39-3.54 (m, 2 H) 3.22 (q, J = 7.1 Hz, 2 H) 2.85-3.03 (m, 4 H) 2.20- |
| | 2.38 (m, 2 H) 2.12 (br d, J = 6.3 Hz, 4 H) 1.51 (br d, J = 13.3 Hz, 2 H) 1.21 (t, J = 7.4 Hz, 3 H) 1.12 |
| | (s, 3 H) |
| 27 | 1H NMR (400 MHz, DMSO-d6) δ 10.90 (s, 1H), 10.12-10.08 (br, 1H), 8.17 (d, J = 2.4 Hz, 1H), |
| | 7.90 (d, J = 8.4 Hz, 1H), 7.81 (s, 1H), 7.68-7.61 (m, 2H), 7.10 (s, 1H), 7.04-6.96 (m, 2H), 6.55 |
| | (s, 1H), 5.64-5.48 (m, 2H), 5.07-4.86 (br, 1H), 3.79-3.76 (m, 2H), 3.67 (s, 2H), 3.37-3.34 |
| | (m, 2H), 3.21-3.18 (m, 2H), 2.71-2.65 (m, 2H), 2.25-2.17 (m, 2H), 2.08-2.05 (m, 2H), 1.91- |
| | 1.78 (m, 2H), 1.68-1.62 (m, 1H), 1.54-1.51 (m, 2H). |
| 28 | 1H NMR (400 MHz, DMSO-d6) δ 12.52 (s, 1H), 10.19 (br, 1H), 8.17-8.11 (m, 2H), 7.80 (s, |
| | 1H), 7.65 (d, J = 8.4 Hz, 1H), 7.36 (d, J = 4.2, 1H), 7.24-7.13 (m, 3H), 6.55 (s, 1H), 5.50 (s, |
| | 2H), 4.95 (br, 1H), 3.79-3.75 (m, 2H), 3.47-3.43 (m, 2H), 3.37-3.33 (m, 2H), 3.13-3.10 (m, |
| | 2H), 2.81-2.75 (m, 2H), 2.27-2.26 (m, 2H), 2.18 (s, 2H), 1.97-1.89 (m, 2H), 1.82-1.79 (m, |
| | 2H), 1.73 (s, 1H). |
| 29 | ¹H NMR (400 MHz, DMSO-d6) δ 10.83 (s, 1H), 10.15 (s, 1H), 8.18 (d, J = 2.0 Hz, 1H), 7.87 (d, |
| | J = 8.8 Hz, 1H), 7.81 (d, J = 1.6 Hz, 1H), 7.64 (s, 2H), 7.14 (s, 1H), 6.99-7.03 (m, 2H), 6.55 (d, J = |
| | 4.0 Hz, 1H), 5.78-5.66 (m, 1H), 5.59-5.48 (m, 1H), 4.95 (s, 1H), 3.82-3.69 (m, 4H), 3.38-3.35 |
| | (m, 2H), 3.08-3.03 (m, 2H), 2.95-2.85 (m, 2H), 2.23-2.17 (m, 2H), 2.03-1.97 (m, 2H), 1.92-1.88 |
| | (m, 2H), 1.23-1.17 (m, 2H), 1.00 (m, 3H). |
| 30 | 1H NMR (400 MHz, DMSO-d6) δ 12.51 (s, 1H), 10.19 (s, 1H), 8.21-8.13 (m, 2H), 7.81 (s, 1H), |
| | 7.66 (d, J = 8.0 Hz, 1H), 7.34-7.31 (m, 2H), 7.22-7.14 (m, 2H), 6.55 (s, 1H), 5.68-5.53 (m, 2H), |
| | 4.95 (s, 1H), 3.82-3.74 (m, 2H), 3.53-3.42 (m, 2H), 3.41-3.35 (m, 2H), 3.01-2.88 (m, 4H), 2.38- |
| | 2.24 (m, 2H), 2.22-2.08 (m, 4H), 1.59-1.48 (m, 2H), 1.23 (m, 3H). |
| 31 | 1H NMR (400 MHz, DMSO-d6) δ 11.18 (s, 1H), 10.20 (s, 1H), 8.61 (s, 1H), 8.58-8.57 (m, 1H), |
| | 8.53-8.50 (m, 1H), 7.92-7.90 (m, 2H), 7.10 (s, 1H), 7.04 (dd, J = 8.4 Hz, 2.0 Hz, 1H), 6.64 (s, |
| | 1H), 5.64-5.51 (m, 2H), 4.95 (s, 1H), 3.78-3.75 (m, 4H), 3.37-3.34 (m, 2H), 3.20-3.17 (m, 2H), |
| | 2.72-2.66 (m, 2H), 2.32-2.26 (m, 2H), 2.07-2.05 (m, 2H), 1.81-1.66 (m, 3H), 1.60-1.53 (m, 2H). |
| 32 | 1H NMR (400 MHz, DMSO-d6) δ 12.88 (s, 1H), 8.63-8.58 (m, 2H), 8.32 (s, 1H), 8.14 (d, J = 8.8 |
| | Hz, 1H), 7.89 (s, 1H), 7.25 (s, 1H), 7.15 (dd, J = 8.8 Hz, 2.0 Hz, 1H), 6.64 (t, J = 2.4 Hz, 1H), |
| | 5.54-5.51 (m, 2H), 3.78-3.75 (m, 2H), 3.54-3.51 (m, 2H), 3.38-3.34 (m, 2H), 3.14-3.10 (m, 2H), |
| | 2.83-2.77 (m, 2H), 2.36-2.31 (m, 2H), 2.20-2.17 (m, 2H), 1.94-1.81 (m, 4H), 1.80-1.70 (m, 1H). |
| 33 | ¹H NMR (400 MHz, DMSO-d₆) δ 10.93 (s, 1 H) 8.18 (d, J = 1.5 Hz, 1 H) 7.93 (d, J = 8.5 Hz, 1 H) |
| | 7.82 (s, 1 H) 7.61-7.70 (m, 2 H) 7.21-7.30 (m, 2 H) 7.00 (br t, J = 6.9 Hz, 1 H) 6.53-6.59 (m, 1 |
| | H) 5.46-5.67 (m, 2 H) 5.02-5.10 (m, 1 H) 3.61-3.81 (m, 5 H) 3.32 (br s, 3 H) 3.15-3.26 (m, 3 |
| | H) 2.75 (br t, J = 11.5 Hz, 2 H) 2.18 (br dd, J = 3.8, 1.5 Hz, 2 H) 2.07 (br d, J = 3.0 Hz, 2 H) 1.73- |
| | 1.91 (m, 2 H) 1.59-1.71 (m, 1 H) 1.49-1.58 (m, 2 H) |
| 34 | ¹H NMR (400 MHz, DMSO-d₆) δ 12.67 (s, 1 H) 8.13-8.24 (m, 2 H) 7.81 (s, 1 H) 7.67 (d, J = 8.3 |
| | Hz, 1 H) 7.49 (d, J = 1.8 Hz, 1 H) 7.32-7.44 (m, 2 H) 7.21 (br t, J = 6.3 Hz, 1 H) 6.56 (d, J = 2.0 Hz, |
| | 1 H) 5.43-5.59 (m, 2 H) 5.05 (t, J = 5.3 Hz, 1 H) 3.74 (q, J = 5.8 Hz, 2 H) 3.41-3.55 (m, 3 H) 3.36 |
| | (br s, 3 H) 3.04-3.22 (m, 3 H) 2.90 (br t, J = 11.1 Hz, 2 H) 2.23-2.33 (m, 2 H) 2.19 (br t, J = 6.9 |
| | Hz, 2 H) 1.87-2.02 (m, 2 H) 1.66-1.86 (m, 3 H) |
| 35 | ¹H NMR (400 MHz, DMSO-d₆) δ 10.87 (s, 1 H) 8.19 (d, J = 2.5 Hz, 1 H) 7.91 (d, J = 8.5 Hz, 1 H) |
| | 7.81 (d, J = 1.8 Hz, 1 H) 7.65 (s, 2 H) 7.33 (d, J = 2.0 Hz, 1 H) 7.24 (dd, J = 8.6, 1.9 Hz, 1 H) 7.04 |
| | (br t, J = 6.6 Hz, 1 H) 6.56 (t, J = 2.1 Hz, 1 H) 5.43-5.80 (m, 2 H) 5.06 (t, J = 5.4 Hz, 1 H) 3.65- |
| | 3.81 (m, 4 H) 3.36 (br s, 2 H) 3.32-3.33 (m, 3 H) 3.03 (br s, 2 H) 2.90-2.99 (m, 2 H) 2.20 (br d, |
| | J = 4.0 Hz, 2 H) 1.95-2.05 (m, 2 H) 1.88-1.94 (m, 2 H) 1.19 (br d, J = 12.5 Hz, 2 H) 1.01 (s, 3 H) |
| 36 | ¹H NMR (400 MHz, DMSO-d₆) δ 12.70 (s, 1 H) 8.10-8.30 (m, 2 H) 7.81 (d, J = 2.0 Hz, 1 H) 7.54- |
| | 7.73 (m, 2 H) 7.40 (dd, J = 8.6, 1.9 Hz, 1 H) 7.34 (d, J = 8.0 Hz, 1 H) 7.23 (br t, J = 6.4 Hz, 1 H) |
| | 6.56 (t, J = 1.9 Hz, 1 H) 5.48-5.67 (m, 2 H) 5.06 (t, J = 5.3 Hz, 1 H) 3.75 (q, J = 5.9 Hz, 2 H) 3.35- |
| | 3.54 (m, 7 H) 3.02-3.14 (m, 2 H) 2.93 (br d, J = 11.3 Hz, 2 H) 2.22-2.35 (m, 2 H) 2.05-2.19 (m, |
| | 4 H) 1.52 (br d, J = 13.3 Hz, 2 H) 1.16 (s, 3 H) |
| 37 | ¹H NMR (400 MHz, DMSO-d₆) δ 10.58 (s, 1 H) 10.16 (br s, 1 H) 8.18 (d, J = 2.3 Hz, 1 H) 7.88 (d, |
| | J = 8.5 Hz, 1 H) 7.78-7.84 (m, 1 H) 7.60-7.69 (m, 2 H) 7.09-7.14 (m, 1 H) 7.01-7.09 (m, 2 H) |
| | 6.56 (t, J = 2.1 Hz, 1 H) 5.51-5.76 (m, 2 H) 4.96 (br s, 1 H) 3.77 (br t, J = 6.4 Hz, 2 H) 3.63-3.72 |
| | (m, 2 H) 3.37 (t, J = 6.5 Hz, 2 H) 3.28 (br d, J = 12.3 Hz, 2 H) 2.87 (br t, J = 11.9 Hz, 2 H) 2.47 (br d, |
| | J = 7.5 Hz, 2 H) 2.16-2.27 (m, 2 H) 1.98-2.12 (m, 2 H) 1.86 (br d, J = 13.3 Hz, 2 H) |
| 38 | ¹H NMR (400 MHz, DMSO-d₆) δ 12.01 (s, 1 H) 10.21 (br s, 1 H) 8.09-8.21 (m, 2 H) 7.81 (d, |
| | J = 1.8 Hz, 1 H) 7.67 (d, J = 8.3 Hz, 1 H) 7.23-7.31 (m, 3 H) 7.20 (dd, J = 8.6, 2.1 Hz, 1 H) 6.56 (t, |
| | J = 2.1 Hz, 1 H) 5.51-5.78 (m, 2 H) 4.96 (br s, 1 H) 3.77 (br t, J = 6.5 Hz, 2 H) 3.35-3.48 (m, 4 H) |
| | 3.23 (br d, J = 11.8 Hz, 2 H) 2.94 (br t, J = 11.9 Hz, 2 H) 2.59 (br d, J = 8.0 Hz, 2 H) 2.26-2.40 (m, |
| | 4 H) 2.12 (br d, J = 13.8 Hz, 2 H) |
| 39 | ¹H NMR (400 MHz, DMSO-d₆) δ 11.15 (br d, J = 3.5 Hz, 1 H) 10.24 (br s, 1 H) 8.44-8.65 (m, 3 |
| | H) 7.80-7.98 (m, 2 H) 6.95-7.20 (m, 2 H) 6.62 (br s, 1 H) 5.41-5.72 (m, 2 H) 3.74 (br s, 2 H) |
| | 3.12-3.28 (m, 4 H) 2.68 (br t, J = 11.3 Hz, 2 H) 2.25 (br s, 2 H) 2.05 (br d, J = 2.8 Hz, 2 H) 1.58- |
| | 1.83 (m, 3 H) 1.52 (br d, J = 11.8 Hz, 2 H) 1.21 (br t, J = 7.3 Hz, 3 H) |
| 40 | ¹H NMR (400 MHz, DMSO-d₆) δ 12.82 (s, 1H), 10.30 (s, 1H), 8.63-8.57 (m, 2H), 8.30 (d, J = |
| | 2.4 Hz, 1H), 8.14-8.12 (m, 1H), 7.89 (s, 1H), 7.26 (d, J = 2.4 Hz, 1H), 7.18-7.15 (m, 1H), 6.63 |
| | (s, 1H), 5.51 (s, 2H), 3.51 (s, 2H), 3.26-3.20 (m, 2H), 3.13-3.10 (m, 2H), 2.82-2.76 (m, 2H), |
| | 2.34 (s, 2H), 2.17 (s, 2H), 1.99-1.64 (m, 5H), 1.24-1.20 (m, 3H). |
| 41 | ¹H NMR (400 MHz, DMSO-d₆) δ 11.08 (s, 1 H) 9.71-10.50 (m, 1 H) 8.47-8.64 (m, 3 H) 7.83- |
| | 7.96 (m, 2 H) 7.16 (s, 1 H) 7.04 (d, J = 8.5 Hz, 1 H) 6.63 (d, J = 1.8 Hz, 1 H) 5.44-5.74 (m, 2 H) |
| | 4.60-5.31 (m, 1 H) 3.78 (br t, J = 6.4 Hz, 4 H) 3.37 (br t, J = 6.5 Hz, 2 H) 3.01 (br d, J = 11.0 Hz, 2 |
| | H) 2.81-2.95 (m, 2 H) 2.27 (br d, J = 4.8 Hz, 2 H) 1.83-2.01 (m, 4 H) 1.18 (br d, J = 12.8 Hz, 2 |
| | H) 0.99 (s, 3 H) |
| 42 | ¹H NMR (400 MHz, DMSO-d₆) δ 12.79 (s, 1 H) 10.23 (br s, 1 H) 8.51-8.69 (m, 2 H) 8.28 (s, 1 |
| | H) 8.16 (d, J = 8.8 Hz, 1 H) 7.89 (d, J = 1.5 Hz, 1 H) 7.35 (d, J = 2.0 Hz, 1 H) 7.17 (dd, J = 8.6, 1.9 |
| | Hz, 1 H) 6.58-6.69 (m, 1 H) 5.50-5.69 (m, 2 H) 4.96 (br s, 1 H) 3.78 (br t, J = 6.4 Hz, 2 H) 3.45- |
| | 3.57 (m, 2 H) 3.38 (br t, J = 6.5 Hz, 3 H) 2.96 (br d, J = 8.0 Hz, 4 H) 2.31-2.44 (m, 2 H) 2.04- |
| | 2.19 (m, 3 H) 1.53 (br d, J = 12.8 Hz, 2 H) 1.13 (s, 3 H) |
| 43 | 1H NMR (400 MHz, DMSO-d6) δ 10.72 (s, 1H), 8.20 (d, J = 2.5 Hz, 1H), 8.06 (d, J = 8.1 Hz, |
| | 1H), 7.82 (d, J = 1.8 Hz, 1H), 7.73-7.65 (m, 1H), 7.65 (d, J = 10.1 Hz, 2H), 7.04 (t, J = 6.7 Hz, |
| | 1H), 6.57 (t, J = 2.2 Hz, 1H), 5.67-5.45 (m, 2H), 4.93 (t, J = 5.4 Hz, 1H), 3.78-3.66 (m, 2H), |
| | 3.55 (dd, J = 9.4, 6.1 Hz, 2H), 3.54-3.43 (m, 1H), 3.28 (d, J = 10.8 Hz, 2H), 3.17 (d, J = 11.3 |
| | Hz, 1H), 2.97 (t, J = 11.3 Hz, 1H), 2.81 (t, J = 11.7 Hz, 2H), 2.18 (d, J = 5.4 Hz, 2H), 2.06 (dd, J = |
| | 7.5, 3.7 Hz, 2H), 1.81 (td, J = 13.0, 12.5, 8.9 Hz, 2H), 1.68 (d, J = 12.7 Hz, 1H), 1.53 (d, J = |
| | 12.3 Hz, 2H). |
| 47 | 1H NMR (400 MHz, DMSO) δ 10.85 (s, 1H), 8.19 (d, J = 2.4 Hz, 1H), 7.93 (d, J = 8.4 Hz, 1H), |
| | 7.81 (s, 1H), 7.65 (s, 2H), 7.25-7.14 (m, 2H), 7.04 (t, J = 6.8 Hz, 1H), 6.56 (t, J = 2.2 Hz, 1H), |
| | 5.76-5.66 (m, 1H), 5.56-5.46 (m, 1H), 5.02 (s, 2H), 4.12-4.06 (m, 2H), 3.99-3.93 (m, 2H), 3.78- |
| | 3.67 (m, 2H), 3.07-2.91 (m, 4H), 2.23-2.16 (m, 2H), 2.05-1.95 (m, 2H), 1.91 (d, J = 7.6 Hz, 2H), |
| | 1.18 (d, J = 13.0 Hz, 2H), 1.01 (s, 3H). |
| 48 | ¹H NMR (400 MHz, DMSO) δ 12.71 (s, 1H), 8.24-8.07 (m, 2H), 7.81 (d, J = 1.9 Hz, 1H), 7.67 |
| | (d, J = 8.2 Hz, 1H), 7.52 (d, J = 2.2 Hz, 1H), 7.43-7.30 (m, 2H), 7.23 (t, J = 6.6 Hz, 1H), 6.56 (t, |
| | J = 2.2 Hz, 1H), 5.63-5.50 (m, 2H), 5.04 (s, 2H), 4.14-3.96 (m, 4H), 3.45 (s, 2H), 3.10 (t, J = |
| | 12.1 Hz, 2H), 2.92 (d, J = 11.5 Hz, 2H), 2.35-2.26 (m, 2H), 2.15-2.11 (m, 3H), 2.08 (s, 1H), 1.52 |
| | (d, J = 13.1 Hz, 2H), 1.17 (s, 3H) |
| 49 | ¹H NMR (400 MHz, DMSO-d₆) δ 10.90 (s, 1 H) 8.18 (d, J = 2.3 Hz, 1 H) 7.95 (d, J = 8.5 Hz, 1 H) |
| | 7.82 (d, J = 1.8 Hz, 1 H) 7.59-7.71 (m, 2 H) 7.19 (dd, J = 8.5, 1.8 Hz, 1 H) 7.14 (d, J = 2.0 Hz, 1 H) |
| | 7.00 (br t, J = 6.8 Hz, 1 H) 6.56 (t, J = 2.0 Hz, 1 H) 5.47-5.67 (m, 2 H) 5.02 (s, 2 H) 3.91-4.12 (m, |
| | 4 H) 3.63-3.74 (m, 2 H) 3.21 (br d, J = 11.0 Hz, 2 H) 2.76 (br t, J = 11.1 Hz, 2 H) 2.12-2.22 (m, 2 |
| | H) 2.06 (br dd, J = 7.1, 3.4 Hz, 2 H) 1.74-1.89 (m, 2 H) 1.60-1.72 (m, 1 H) 1.54 (br d, J = 12.0 |
| | Hz, 2 H) |
| 52 | ¹H NMR (400 MHz, DMSO-d₆) δ 11.09 (s, 1 H) 8.51-8.64 (m, 2 H) 8.47 (d, J = 2.5 Hz, 1 H) 7.89 |
| | (d, J = 8.8 Hz, 1 H) 7.15 (d, J = 1.8 Hz, 1 H) 7.02 (dd, J = 8.5, 2.0 Hz, 1 H) 6.55 (d, J = 2.5 Hz, 1 H) |
| | 5.44-5.76 (m, 2 H) 5.28 (br s, 1 H) 4.58 (s, 2 H) 3.77 (br t, J = 6.5 Hz, 4 H) 3.33-3.38 (m, 4 H) |
| | 2.96-3.06 (m, 2 H) 2.88 (br t, J = 11.8 Hz, 2 H) 2.21-2.35 (m, 2 H) 1.81-2.03 (m, 4 H) 1.19 (br |
| | d, J = 12.5 Hz, 2 H) 1.00 (s, 3 H) |
| 53 | ¹H NMR (400 MHz, DMSO-d₆) δ 12.63-12.82 (m, 1 H) 8.60-8.79 (m, 1 H) 8.49 (d, J = 2.3 Hz, |
| | 1 H) 8.00-8.34 (m, 2 H) 7.18-7.38 (m, 1 H) 7.05-7.18 (m, 1 H) 6.41-6.63 (m, 1 H) 5.42- |
| | 5.73 (m, 3 H) 5.27 (t, J = 5.9 Hz, 1 H) 4.41-4.68 (m, 3 H) 3.66-3.86 (m, 3 H) 3.42-3.61 (m, 4 |
| | H) 2.78-3.06 (m, 7 H) 1.99-2.21 (m, 3 H) 1.42-1.58 (m, 2 H) 1.03-1.13 (m, 2 H) |
| 54 | ¹H NMR (400 MHz, DMSO) δ 10.90 (s, 1H), 8.07 (s, 1H), 7.89 (d, J = 8.5 Hz, 1H), 7.62 (dd, |
| | 2H), 7.11-6.96 (m, 3H), 6.48 (s, 1H), 5.62-5.51 (m, 3H), 5.16 (t, J = 5.8 Hz, 1H), 4.53 (d, J = |
| | 5.8 Hz, 2H), 3.76 (s, 1H), 3.68 (s, 2H), 3.19 (d, J = 11.2 Hz, 2H), 2.72-2.64 (m, 3H), 2.32 (d, J = |
| | 11.6 Hz, 2H), 2.18 (s, 2H), 2.06 (s, 2H), 1.84-1.75 (m, 1H), 1.67 (s, 2H), 1.54 (d, J = 12.6 Hz, |
| | 3H). |
| 56 | 1H NMR (400 MHz, DMSO-d6) δ 12.68 (s, 1 H) 8.16 (d, J = 2.3 Hz, 1 H) 8.01-8.09 (m, 1 H) |
| | 7.80 (d, J = 1.8 Hz, 1 H) 7.56-7.68 (m, 2 H) 6.99-7.27 (m, 4 H) 6.50-6.60 (m, 1 H) 5.33-5.59 |
| | (m, 2 H) 4.40 (br t, J = 5.4 Hz, 2 H) 4.18 (br d, J = 3.8 Hz, 2 H) 3.70-3.81 (m, 3 H) 3.11 (br d, |
| | J = 11.3 Hz, 3 H) 2.88 (br t, J = 12.1 Hz, 3 H) 2.53-2.59 (m, 2 H) 1.70 (br d, J = 12.0 Hz, 2 H) 1.32 |
| | (s, 3 H) |
| 57 | ¹H NMR (400 MHz, DMSO-d₆) δ 11.19-11.31 (m, 1 H) 8.27-8.38 (m, 1 H) 8.20 (d, J = 2.5 Hz, |
| | 1 H) 7.76-7.89 (m, 2 H) 7.57-7.71 (m, 2 H) 7.20-7.33 (m, 1 H) 7.06 (s, 1 H) 6.98 (br d, J = 9.3 |
| | Hz, 1 H) 6.47-6.61 (m, 1 H) 5.93-6.08 (m, 1 H) 5.47-5.61 (m, 1 H) 4.07-4.18 (m, 2 H) 3.85- |
| | 3.93 (m, 3 H) 3.72-3.79 (m, 3 H) 3.08-3.17 (m, 4 H) 2.73-2.85 (m, 3 H) 1.60-1.70 (m, 2 H) |
| | 1.22 (s, 3 H) |
| 58 | ¹H NMR (400 MHz, DMSO-d₆) δ 10.75 (s, 1 H) 8.19 (d, J = 2.3 Hz, 1 H) 8.03 (d, J = 8.3 Hz, 1 H) |
| | 7.75-7.86 (m, 2 H) 7.66 (s, 2 H) 7.51-7.61 (m, 2 H) 7.03 (br t, J = 6.8 Hz, 1 H) 6.57 (t, J = 2.1 Hz, |
| | 1 H) 5.43-5.67 (m, 2 H) 4.66-4.77 (m, 1 H) 3.63-3.76 (m, 2 H) 3.39 (br d, J = 5.8 Hz, 2 H) 3.27 |
| | (br d, J = 11.5 Hz, 2 H) 2.72-2.88 (m, 4 H) 2.18 (br d, J = 4.5 Hz, 2 H) 2.06 (br dd, J = 7.4, 3.4 Hz, |
| | 2 H) 1.74-1.88 (m, 2 H) 1.60-1.72 (m, 1 H) 1.54 (br d, J = 12.5 Hz, 2 H) |
| 59 | ¹H NMR (400 MHz, DMSO-d₆) δ 12.57 (s, 1 H) 8.34 (d, J = 8.3 Hz, 1 H) 8.19 (d, J = 2.0 Hz, 1 H) |
| | 7.78-7.92 (m, 3 H) 7.62-7.76 (m, 2 H) 7.36 (d, J = 8.0 Hz, 1 H) 7.25 (t, J = 6.5 Hz, 1 H) 6.48- |
| | 6.65 (m, 1 H) 5.42-5.59 (m, 2 H) 4.65-4.85 (m, 1 H) 3.43-3.53 (m, 2 H) 3.37-3.41 (m, 2 H) |
| | 3.12-3.21 (m, 2 H) 2.80-2.97 (m, 4 H) 2.23-2.31 (m, 2 H) 2.19 (br t, J = 7.3 Hz, 2 H) 1.87- |
| | 2.02 (m, 2 H) 1.69-1.87 (m, 3 H) |
| 60 | ¹H NMR (400 MHz, DMSO-d₆) δ 10.96 (s, 1 H) 10.15 (br s, 1 H) 8.22-8.38 (m, 1 H) 7.89 (d, |
| | J = 8.8 Hz, 1 H) 7.52-7.76 (m, 2 H) 6.95-7.15 (m, 3 H) 6.39 (br t, J = 6.4 Hz, 1 H) 5.43-5.70 (m, |
| | 2 H) 4.97 (br s, 1 H) 3.76 (br t, J = 6.3 Hz, 2 H) 3.63 (br s, 2 H) 3.34-3.38 (m, 4 H) 3.20 (br d, |
| | J = 10.8 Hz, 2 H) 2.69 (br t, J = 11.4 Hz, 2 H) 2.16 (br d, J = 2.5 Hz, 2 H) 2.02-2.10 (m, 3 H) 1.74- |
| | 1.90 (m, 2 H) 1.61-1.72 (m, 1 H) 1.54 (br d, J = 11.5 Hz, 2 H) |
| 61 | ¹H NMR (400 MHz, DMSO-d₆) δ 12.49-12.69 (m, 1 H) 8.17-8.36 (m, 1 H) 7.98-8.15 (m, 1 |
| | H) 7.53-7.73 (m, 1 H) 7.31-7.47 (m, 1 H) 6.94-7.29 (m, 4 H) 6.51-6.76 (m, 1 H) 5.35-5.61 |
| | (m, 2 H) 3.65-3.87 (m, 4 H) 3.41-3.48 (m, 3 H) 3.06-3.15 (m, 3 H) 2.73-2.90 (m, 3 H) 2.12- |
| | 2.31 (m, 4 H) 1.65-2.04 (m, 6 H) |
| 62 | ¹H NMR (400 MHz, DMSO) δ 11.17 (s, 1H), 8.58 (s, 1H), 7.90 (d, J = 8.6 Hz, 1H), 7.75 (d, J = |
| | 1.8 Hz, 1H), 7.50 (t, J = 6.7 Hz, 1H), 7.16 (d, J = 2.1 Hz, 1H), 7.02 (dd, J = 8.5, 2.0 Hz, 1H), 6.51 |
| | (d, J = 1.7 Hz, 1H), 5.67 (dt, J = 15.4, 7.6 Hz, 1H), 5.57-5.46 (m, 1H), 5.36-5.26 (m, 2H), 4.69 |
| | (d, J = 5.7 Hz, 2H), 3.77 (t, J = 6.5 Hz, 2H), 3.68 (d, J = 5.7 Hz, 2H), 3.01 (d, J = 11.3 Hz, 2H), |
| | 2.89 (t, J = 12.1 Hz, 2H), 2.23 (d, J = 5.7 Hz, 2H), 2.07-1.86 (m, 5H), 1.47 (d, J = 7.6 Hz, 2H), |
| | 1.01 (s, 3H), 0.90-0.82 (m, 2H). |
| 63 | ¹H NMR (400 MHz, DMSO) δ 10.96 (s, 1H), 8.31 (d, J = 2.6 Hz, 1H), 7.95 (d, J = 8.4 Hz, 1H), |
| | 7.72-7.60 (m, 2H), 7.23-7.12 (m, 2H), 7.06 (d, J = 2.6 Hz, 1H), 6.42 (t, J = 6.6 Hz, 1H), 5.67- |
| | 5.47 (m, 2H), 5.02 (s, 2H), 4.12-4.05 (m, 2H), 3.99-3.92 (m, 2H), 3.63 (d, J = 6.9 Hz, 2H), |
| | 3.35 (s, 3H), 3.21 (d, J = 11.2 Hz, 2H), 2.81-2.71 (m, 2H), 2.16 (d, J = 4.9 Hz, 2H), 2.07 (d, J = |
| | 9.2 Hz, 2H), 1.88-1.75 (m, 2H), 1.69-1.64 (m, 1H), 1.54 (d, J = 12.2 Hz, 2H). |
| 64 | ¹H NMR (400 MHz, DMSO-d₆) δ 12.78 (s, 1 H) 8.31 (d, J = 2.5 Hz, 1 H) 8.20 (d, J = 8.5 Hz, 1 H) |
| | 7.67 (d, J = 8.3 Hz, 1 H) 7.41 (d, J = 8.3 Hz, 1 H) 7.38 (s, 1 H) 7.32-7.36 (m, 1 H) 7.06 (d, J = 2.3 |
| | Hz, 1 H) 6.68 (br t, J = 6.1 Hz, 1 H) 5.43-5.60 (m, 2 H) 5.04 (s, 2 H) 4.04-4.16 (m, 2 H) 3.93- |
| | 4.03 (m, 2 H) 3.39-3.50 (m, 2 H) 3.36 (s, 3 H) 3.11 (br d, J = 11.0 Hz, 2 H) 2.91 (br t, J = 11.0 Hz, |
| | 2 H) 2.13-2.30 (m, 4 H) 1.66-1.99 (m, 5 H) |
| 68 | ¹H NMR (499 MHz, DMSO-d₆) δ 10.85 (d, J = 14.5 Hz, 2 H) 8.17 (d, J = 1.9 Hz, 1 H) 7.90 (d, |
| | J = 8.5 Hz, 1 H) 7.80 (d, J = 1.6 Hz, 1 H) 7.64 (q, J = 8.5 Hz, 2 H) 7.09 (d, J = 2.2 Hz, 1 H) 7.03 (dd, |
| | J = 8.6, 2.1 Hz, 1 H) 6.97 (br t, J = 6.7 Hz, 1 H) 6.52-6.57 (m, 1 H) 5.46-5.65 (m, 2 H) 4.67 (q, |
| | J = 9.7 Hz, 2 H) 3.67 (br d, J = 3.8 Hz, 2 H) 3.21 (br d, J = 11.5 Hz, 2 H) 2.69 (br t, J = 11.2 Hz, 2 H) |
| | 2.12-2.21 (m, 2 H) 2.05 (dd, J = 7.4, 3.6 Hz, 2 H) 1.81 (qd, J = 12.4, 3.3 Hz, 2 H) 1.60-1.71 (m, 1 |
| | H) 1.53 (br d, J = 11.0 Hz, 2 H) |
| 69 | ¹H NMR (499 MHz, DMSO-d₆) δ 11.05 (br s, 1 H) 10.95 (br s, 1 H) 8.16 (d, J = 2.2 Hz, 1 H) 7.87 |
| | (d, J = 8.8 Hz, 1 H) 7.80 (d, J = 1.6 Hz, 1 H) 7.64-7.68 (m, 1 H) 7.59-7.63 (m, 1 H) 7.04 (br s, 1 |
| | H) 6.92-7.00 (m, 2 H) 6.52-6.57 (m, 1 H) 5.47-5.66 (m, 2 H) 4.75-4.95 (m, 2 H) 3.61-3.72 |
| | (m, 2 H) 3.19 (br d, J = 10.4 Hz, 2 H) 2.65-2.74 (m, 2 H) 2.13-2.21 (m, 2 H) 2.05 (dd, J = 7.1, 3.6 |
| | Hz, 2 H) 1.74-1.88 (m, 2 H) 1.60-1.72 (m, 1 H) 1.53 (br d, J = 11.8 Hz, 2 H) |
| 71 | ¹H NMR (499 MHz, DMSO-d₆) δ 10.83 (s, 1 H) 10.63 (br s, 1 H) 9.21-9.42 (m, 1 H) 9.04-9.19 |
| | (m, 1 H) 8.16 (d, J = 2.2 Hz, 1 H) 7.91 (d, J = 8.5 Hz, 1 H) 7.81 (d, J = 1.9 Hz, 1 H) 7.58-7.69 (m, 2 |
| | H) 7.04 (d, J = 2.2 Hz, 1 H) 6.95-7.03 (m, 2 H) 6.53-6.57 (m, 1 H) 5.47-5.64 (m, 2 H) 4.53 (tt, |
| | J = 8.9, 6.3 Hz, 1 H) 4.24-4.40 (m, 2 H) 4.13 (br s, 2 H) 3.67 (br d, J = 3.3 Hz, 2 H) 3.19 (br d, |
| | J = 11.5 Hz, 2 H) 2.64-2.75 (m, 2 H) 2.12-2.22 (m, 2 H) 2.06 (dd, J = 7.3, 3.4 Hz, 2 H) 1.81 (qd, |
| | J = 12.2, 3.3 Hz, 2 H) 1.59-1.73 (m, 1 H) 1.54 (br d, J = 11.2 Hz, 2 H) |
| 73 | ¹H NMR (499 MHz, DMSO-d₆) δ 10.94 (br s, 1 H) 10.42 (br d, J = 3.0 Hz, 1 H) 8.16 (d, J = 2.2 Hz, |
| | 1 H) 7.84 (d, J = 8.8 Hz, 1 H) 7.80 (d, J = 1.4 Hz, 1 H) 7.58-7.68 (m, 2 H) 7.00 (br s, 1 H) 6.91- |
| | 6.97 (m, 2 H) 6.54 (t, J = 2.2 Hz, 1 H) 5.47-5.64 (m, 2 H) 4.74-4.80 (m, 2 H) 4.65-4.73 (m, 3 |
| | H) 3.62-3.71 (m, 2 H) 3.17 (br d, J = 11.2 Hz, 2 H) 2.64-2.72 (m, 2 H) 2.12-2.21 (m, 2 H) 2.02- |
| | 2.09 (m, 2 H) 1.75-1.87 (m, 2 H) 1.60-1.72 (m, 1 H) 1.47-1.58 (m, 2 H) |
| 74 | ¹H NMR (499 MHz, DMSO-d₆) δ 10.91 (s, 1 H) 10.31 (br s, 1 H) 8.16 (d, J = 2.2 Hz, 1 H) 7.88 (d, |
| | J = 8.5 Hz, 1 H) 7.80 (d, J = 1.6 Hz, 1 H) 7.58-7.70 (m, 2 H) 7.06 (d, J = 1.9 Hz, 1 H) 7.01 (dd, |
| | J = 8.6, 2.1 Hz, 1 H) 6.96 (t, J = 6.7 Hz, 1 H) 6.52-6.56 (m, 1 H) 5.47-5.64 (m, 2 H) 3.63-3.70 |
| | (m, 2 H) 3.38 (t, J = 7.4 Hz, 2 H) 3.19 (br d, J = 11.8 Hz, 2 H) 2.93 (t, J = 2.6 Hz, 1 H) 2.64-2.72 |
| | (m, 2 H) 2.60 (td, J = 7.3, 2.7 Hz, 2 H) 2.13-2.20 (m, 2 H) 2.05 (dd, J = 7.4, 3.8 Hz, 2 H) 1.81 (qd, |
| | J = 12.3, 3.6 Hz, 2 H) 1.59-1.72 (m, 1 H) 1.53 (br d, J = 10.7 Hz, 2 H) |
| 75 | ¹H NMR (499 MHz, DMSO-d₆) δ 10.87 (s, 1 H) 10.38-10.62 (m, 1 H) 8.16 (d, J = 2.2 Hz, 1 H) |
| | 7.92 (d, J = 8.5 Hz, 1 H) 7.81 (d, J = 1.9 Hz, 1 H) 7.57-7.70 (m, 2 H) 7.03-7.09 (m, 2 H) 6.94- |
| | 7.01 (m, 1 H) 6.53-6.57 (m, 1 H) 5.48-5.64 (m, 2 H) 3.28-3.73 (m, 14 H) 3.23-3.23 (m, 1 H) |
| | 3.20 (br d, J = 11.2 Hz, 2 H) 2.68 (br t, J = 11.4 Hz, 2 H) 2.12-2.22 (m, 2 H) 2.06 (dd, J = 7.4, 3.6 |
| | Hz, 2 H) 1.82 (qd, J = 12.4, 3.4 Hz, 2 H) 1.61-1.72 (m, 1 H) 1.54 (br d, J = 10.7 Hz, 2 H) |
| 76 | ¹H NMR (499 MHz, DMSO-d₆) δ 10.87-10.95 (m, 1 H) 10.38-10.45 (m, 1 H) 8.16 (d, J = 2.5 |
| | Hz, 1 H) 7.88 (d, J = 8.5 Hz, 1 H) 7.80 (d, J = 1.6 Hz, 1 H) 7.63 (q, J = 8.4 Hz, 2 H) 7.00-7.05 (m, 1 |
| | H) 6.89-6.99 (m, 2 H) 6.52-6.57 (m, 1 H) 6.33-6.45 (m, 1 H) 5.44-5.68 (m, 2 H) 4.51 (s, 2 |
| | H) 3.58-3.72 (m, 5 H) 3.17 (br d, J = 11.0 Hz, 2 H) 2.61-2.73 (m, 2 H) 2.40 (t, J = 5.9 Hz, 2 H) |
| | 2.11-2.25 (m, 2 H) 2.05 (dd, J = 7.5, 3.4 Hz, 2 H) 1.74-1.87 (m, 2 H) 1.61-1.72 (m, 2 H) 1.48- |
| | 1.72 (m, 2 H) |
| 78 | ¹H NMR (499 MHz, DMSO-d₆) δ 10.89 (s, 1 H) 10.64 (s, 1 H) 8.15 (d, J = 2.2 Hz, 1 H) 7.97 (d, |
| | J = 1.4 Hz, 1 H) 7.77-7.81 (m, 2 H) 7.76 (d, J = 1.1 Hz, 1 H) 7.58-7.64 (m, 2 H) 7.06 (d, J = 1.9 |
| | Hz, 1 H) 6.95 (dd, J = 8.6, 2.1 Hz, 2 H) 6.52-6.56 (m, 1 H) 5.46-5.64 (m, 2 H) 3.62-3.71 (m, 5 |
| | H) 3.11 (br d, J = 11.5 Hz, 2 H) 2.57-2.67 (m, 2 H) 2.12-2.21 (m, 2 H) 2.05 (dd, J = 7.4, 3.6 Hz, 2 |
| | H) 1.73-1.85 (m, 2 H) 1.60-1.72 (m, 1 H) 1.52 (br d, J = 11.0 Hz, 2 H) |
| 80 | ¹H NMR (499 MHz, DMSO-d₆) δ 10.91 (s, 1 H) 10.79 (br s, 1 H) 8.17 (d, J = 2.2 Hz, 1 H) 7.88 (d, |
| | J = 8.5 Hz, 1 H) 7.80 (d, J = 1.6 Hz, 1 H) 7.59-7.67 (m, 2 H) 7.07 (d, J = 1.6 Hz, 1 H) 7.01 (dd, |
| | J = 8.5, 1.9 Hz, 1 H) 6.96 (t, J = 6.7 Hz, 1 H) 6.53-6.56 (m, 1 H) 5.47-5.64 (m, 2 H) 5.24 (br s, 2 |
| | H) 3.62-3.71 (m, 2 H) 3.16-3.24 (m, 5 H) 2.65-2.74 (m, 2 H) 2.11-2.22 (m, 2 H) 2.05 (dd, |
| | J = 7.4, 3.6 Hz, 2 H) 1.81 (qd, J = 12.5, 3.3 Hz, 2 H) 1.60-1.73 (m, 1 H) 1.48-1.57 (m, 2 H) |
| 81 | ¹H NMR (499 MHz, DMSO-d₆) δ 10.88 (s, 1 H) 10.56 (br s, 1 H) 8.33 (s, 1 H) 8.16 (d, J = 2.2 Hz, |
| | 1 H) 7.82 (d, J = 8.5 Hz, 1 H) 7.80 (d, J = 1.6 Hz, 1 H) 7.77 (s, 1 H) 7.58-7.65 (m, 2 H) 7.01 (d, |
| | J = 1.9 Hz, 1 H) 6.92-6.97 (m, 2 H) 6.52-6.56 (m, 1 H) 5.45-5.64 (m, 2 H) 3.84 (s, 3 H) 3.62- |
| | 3.70 (m, 2 H) 3.13 (br d, J = 11.5 Hz, 2 H) 2.58-2.69 (m, 2 H) 2.12-2.20 (m, 2 H) 2.05 (dd, |
| | J = 7.4, 3.6 Hz, 2 H) 1.79 (qd, J = 12.3, 3.6 Hz, 2 H) 1.66 (ddt, J = 11.9, 8.0, 4.0, 4.0 Hz, 1 H) 1.52 |
| | (br d, J = 11.0 Hz, 2 H) |
| 99 | ¹H NMR (400 MHz, DMSO-d₆) δ 11.14 (s, 1 H) 8.21 (d, J = 2.3 Hz, 1 H) 7.89 (d, J = 8.5 Hz, 1 H) |
| | 7.80 (d, J = 1.8 Hz, 1 H) 7.56-7.72 (m, 2 H) 7.27 (t, J = 5.5 Hz, 1 H) 7.23 (d, J = 1.8 Hz, 1 H) 7.04 |
| | (dd, J = 8.6, 1.9 Hz, 1 H) 6.52-6.59 (m, 1 H) 5.36-5.57 (m, 2 H) 3.88 (br s, 2 H) 3.76 (t, J = 6.5 |
| | Hz, 2 H) 2.84-3.05 (m, 4 H) 2.24 (br s, 2 H) 1.92-2.17 (m, 3 H) 1.41-1.58 (m, 2 H) 1.12-1.33 |
| | (m, 5 H) 0.98 (s, 3 H) |
| 100 | ¹H NMR (400 MHz, DMSO-d₆) δ 12.30 (s, 1 H) 8.05-8.21 (m, 2 H) 7.80 (d, J = 1.5 Hz, 1 H) 7.57- |
| | 7.73 (m, 2 H) 7.40 (d, J = 2.0 Hz, 1 H) 7.27 (br t, J = 6.1 Hz, 1 H) 7.14 (dd, J = 8.6, 2.1 Hz, 1 H) |
| | 6.49-6.61 (m, 1 H) 5.10-5.45 (m, 2 H) 4.11 (br s, 2 H) 3.77 (t, J = 6.5 Hz, 2 H) 2.87-3.09 (m, 4 |
| | H) 2.16-2.34 (m, 4 H) 1.93-2.05 (m, 1 H) 1.49-1.61 (m, 2 H) 1.15-1.36 (m, 5 H) 1.07 (s, 3 |
| | H) |
| 101 | ¹H NMR (400 MHz, DMSO-d₆) δ 10.77 (s, 1 H) 8.33 (d, J = 2.5 Hz, 1 H) 8.05 (d, J = 8.0 Hz, 1 H) |
| | 7.61-7.73 (m, 4 H) 7.07 (d, J = 2.3 Hz, 1 H) 6.45 (br t, J = 6.4 Hz, 1 H) 5.42-5.67 (m, 2 H) 4.93 |
| | (br t, J = 5.4 Hz, 1 H) 3.72 (q, J = 5.9 Hz, 2 H) 3.62-3.68 (m, 2 H) 3.53 (br t, J = 6.3 Hz, 2 H) 3.35 |
| | (s, 3 H) 3.25-3.29 (m, 2 H) 2.82 (br t, J = 11.1 Hz, 2 H) 2.11-2.22 (m, 2 H) 2.07 (br dd, J = 7.0, |
| | 3.5 Hz, 2 H) 1.73-1.87 (m, 2 H) 1.61-1.72 (m, 1 H) 1.54 (br d, J = 11.0 Hz, 2 H) |
| 102 | ¹H NMR (400 MHz, DMSO-d₆) δ 12.69 (s, 1 H) 8.37 (d, J = 8.3 Hz, 1 H) 8.32 (d, J = 2.5 Hz, 1 H) |
| | 7.93 (d, J = 0.8 Hz, 1 H) 7.81-7.88 (m, 1 H) 7.70 (d, J = 8.3 Hz, 1 H) 7.40 (d, J = 8.3 Hz, 1 H) 7.07 |
| | (d, J = 2.5 Hz, 1 H) 6.71 (br t, J = 6.5 Hz, 1 H) 5.43-5.61 (m, 2 H) 4.94 (br s, 1 H) 3.69-3.78 (m, |
| | 2 H) 3.52-3.61 (m, 2 H) 3.41-3.49 (m, 2 H) 3.36 (s, 3 H) 3.18 (br d, J = 11.0 Hz, 2 H) 2.97 (br t, |
| | J = 10.8 Hz, 2 H) 2.15-2.31 (m, 4 H) 1.70-1.99 (m, 5 H) |
| 103 | ¹H NMR (400 MHz, DMSO) δ 12.72 (s, 1H), 10.22 (s, 1H), 8.30 (d, J = 2.4 Hz, 1H), 8.05 (d, J = |
| | 8.6 Hz, 1H), 7.66-7.49 (m, 2H), 7.23 (d, J = 1.7 Hz, 1H), 7.12 (d, J = 2.2 Hz, 1H), 7.05 (d, J = |
| | 2.4 Hz, 1H), 6.54 (t, J = 6.2 Hz, 1H), 6.39 (d, J = 5.6 Hz, 1H), 4.96 (s, 1H), 4.74 (q, J = 7.4 Hz, |
| | 1H), 3.77 (t, J = 6.4 Hz, 2H), 3.54-3.44 (m, 2H), 3.39-3.34 (m, 5H), 3.16 (d, J = 11.6 Hz, 2H), |
| | 2.88 (t, J = 12.0 Hz, 2H), 2.33 (d, J = 8.6 Hz, 2H), 2.25-2.15 (m, 2H), 1.86 (d, J = 12.4 Hz, 2H), |
| | 1.44 (s, 3H). |
| 105 | ¹H NMR (400 MHz, DMSO) δ 12.75 (s, 1H), 10.23 (s, 1H), 8.29 (d, J = 2.5 Hz, 1H), 8.07 (d, J = |
| | 8.8 Hz, 1H), 7.68-7.59 (m, 2H), 7.24 (s, 1H), 7.12-7.01 (m, 2H), 6.62 (s, 1H), 5.55-5.46 (m, |
| | 1H), 5.42-5.34 (m, 1H), 4.37 (s, 2H), 4.17 (d, J = 4.7 Hz, 2H), 3.35 (s, 3H), 3.23-3.09 (m, 4H), |
| | 2.88 (t, J = 12.2 Hz, 2H), 2.34-2.30 (m, 2H), 1.71 (d, J = 12.3 Hz, 2H), 1.32 (s, 3H), 1.27-1.17 |
| | (m, 3H). |
| 106 | ¹H NMR (400 MHz, DMSO-d₆) δ 11.30 (s, 1 H) 10.19 (br s, 1 H) 8.34 (d, J = 2.5 Hz, 1 H) 7.86 (d, |
| | J = 8.5 Hz, 1 H) 7.60-7.72 (m, 2 H) 7.11 (s, 1 H) 7.05 (d, J = 2.5 Hz, 1 H) 7.02 (br d, J = 8.3 Hz, 1 |
| | H) 6.81 (br t, J = 5.1 Hz, 1 H) 5.96-6.06 (m, 1 H) 5.51-5.62 (m, 1 H) 4.13 (br s, 2 H) 3.86 (br s, |
| | 2 H) 3.35 (s, 3 H) 3.11-3.23 (m, 4 H) 2.81 (br t, J = 11.8 Hz, 2 H) 2.21-2.31 (m, 2 H) 1.67 (br d, |
| | J = 11.5 Hz, 2 H) 1.18-1.24 (m, 6 H) |
| 107 | ¹H NMR (400 MHz, DMSO-d₆) δ 12.81 (s, 1 H) 8.28-8.36 (m, 2 H) 8.01 (d, J = 1.5 Hz, 1 H) 7.82 |
| | (dd, J = 8.4, 1.6 Hz, 1 H) 7.59-7.72 (m, 2 H) 7.07 (d, J = 2.3 Hz, 1 H) 6.69 (t, J = 6.1 Hz, 1 H) 5.46- |
| | 5.57 (m, 1 H) 5.38 (dt, J = 11.5, 5.8 Hz, 1 H) 4.95 (br s, 1 H) 4.37 (br t, J = 5.4 Hz, 2 H) 4.19 (br |
| | d, J = 3.3 Hz, 2 H) 3.74 (br s, 2 H) 3.53-3.62 (m, 2 H) 3.35 (s, 3 H) 3.05-3.21 (m, 4 H) 2.52- |
| | 2.58 (m, 2 H) 1.71 (br d, J = 12.0 Hz, 2 H) 1.36 (s, 3 H) |
| 108 | ¹H NMR (400 MHz, DMSO-d₆) δ 11.01 (s, 1 H) 8.36 (d, J = 2.5 Hz, 1 H) 8.01 (d, J = 8.0 Hz, 1 H) |
| | 7.60-7.77 (m, 4 H) 7.06 (d, J = 2.5 Hz, 1 H) 6.87 (t, J = 5.1 Hz, 1 H) 6.00 (br d, J = 15.5 Hz, 1 H) |
| | 5.51-5.63 (m, 1 H) 4.93 (br t, J = 5.5 Hz, 1 H) 4.05-4.18 (m, 2 H) 3.88 (br s, 2 H) 3.68-3.78 (m, |
| | 2 H) 3.49-3.59 (m, 2 H) 3.35 (s, 3 H) 3.21-3.27 (m, 2 H) 2.99 (br t, J = 10.9 Hz, 2 H) 2.15-2.26 |
| | (m, 2 H) 1.65 (br d, J = 12.5 Hz, 2 H) 1.23 (s, 3 H) |
| 109 | ¹H NMR (400 MHz, DMSO-d₆) δ 10.90 (s, 1 H) 9.89-10.40 (m, 1 H) 8.04 (s, 1 H) 7.89 (d, J = 8.5 |
| | Hz, 1 H) 7.74 (s, 1 H) 7.56-7.69 (m, 2 H) 7.09 (d, J = 2.0 Hz, 1 H) 6.98-7.05 (m, 2 H) 5.45- |
| | 5.66 (m, 2 H) 4.99 (t, J = 5.4 Hz, 2 H) 4.47 (d, J = 5.3 Hz, 2 H) 3.76 (t, J = 6.6 Hz, 2 H) 3.63-3.72 |
| | (m, 2 H) 3.33-3.37 (m, 2 H) 3.19 (br d, J = 11.0 Hz, 2 H) 2.68 (br t, J = 11.4 Hz, 2 H) 2.17 (br d, |
| | J = 3.8 Hz, 2 H) 2.06 (br dd, J = 7.4, 3.4 Hz, 2 H) 1.74-1.89 (m, 2 H) 1.60-1.72 (m, 1 H) 1.53 (br |
| | d, J = 11.5 Hz, 2 H) |
| 110 | ¹H NMR (400 MHz, DMSO-d₆) δ 12.63 (s, 1 H) 8.75 (s, 1 H) 8.23 (s, 1 H) 8.11 (d, J = 8.5 Hz, 1 |
| | H) 7.66 (d, J = 8.3 Hz, 1 H) 7.38 (d, J = 8.3 Hz, 1 H) 7.23 (d, J = 2.0 Hz, 1 H) 7.13 (dd, J = 8.6, 2.1 |
| | Hz, 1 H) 6.76 (t, J = 6.3 Hz, 1 H) 5.44-5.58 (m, 2 H) 3.76 (t, J = 6.5 Hz, 2 H) 3.38-3.47 (m, 2 H) |
| | 3.35 (s, 4 H) 3.29 (s, 3 H) 3.12 (br d, J = 11.0 Hz, 2 H) 2.79 (br t, J = 10.9 Hz, 2 H) 2.14-2.30 (m, |
| | 4 H) 1.66-1.99 (m, 5 H) |
| 111 | ¹H NMR (400 MHz, DMSO-d₆) δ 10.96 (s, 1 H) 8.76 (s, 1 H) 8.22 (s, 1 H) 7.89 (d, J = 8.5 Hz, 1 |
| | H) 7.56-7.74 (m, 2 H) 7.09 (d, J = 1.8 Hz, 1 H) 7.02 (dd, J = 8.6, 1.9 Hz, 1 H) 6.53 (t, J = 6.6 Hz, 1 |
| | H) 5.47-5.65 (m, 2 H) 3.76 (t, J = 6.5 Hz, 2 H) 3.58-3.68 (m, 2 H) 3.33-3.37 (m, 2 H) 3.29 (s, 3 |
| | H) 3.20 (br d, J = 11.0 Hz, 2 H) 2.63-2.74 (m, 2 H) 2.17 (br d, J = 3.5 Hz, 2 H) 2.06 (br dd, J = 6.9, |
| | 3.4 Hz, 2 H) 1.74-1.88 (m, 2 H) 1.61-1.73 (m, 1 H) 1.54 (br d, J = 11.5 Hz, 2 H) |
| 112 | ¹H NMR (400 MHz, DMSO-d₆) δ 12.52 (s, 1 H) 8.11 (d, J = 8.5 Hz, 1 H) 8.05 (s, 1 H) 7.73 (s, 1 |
| | H) 7.63 (d, J = 8.0 Hz, 1 H) 7.35 (d, J = 8.3 Hz, 1 H) 7.20-7.26 (m, 2 H) 7.13 (br d, J = 8.5 Hz, 1 H) |
| | 5.43-5.57 (m, 2 H) 5.00 (t, J = 5.4 Hz, 1 H) 4.46 (d, J = 5.3 Hz, 2 H) 3.76 (t, J = 6.4 Hz, 2 H) 3.40- |
| | 3.52 (m, 2 H) 3.33-3.37 (m, 4 H) 3.12 (br d, J = 10.5 Hz, 2 H) 2.78 (br t, J = 11.0 Hz, 2 H) 2.23- |
| | 2.31 (m, 2 H) 2.18 (br t, J = 6.6 Hz, 2 H) 1.86-2.02 (m, 2 H) 1.66-1.86 (m, 3 H) |
| 113 | ¹H NMR (400 MHz, DMSO) δ 13.00 (s, 1H), 8.30 (d, J = 2.5 Hz, 1H), 8.18 (d, J = 8.6 Hz, 1H), |
| | 7.70-7.59 (m, 2H), 7.45 (d, J = 2.2 Hz, 1H), 7.34 (dd, J = 8.6, 2.2 Hz, 1H), 7.06 (d, J = 2.5 Hz, |
| | 1H), 6.66 (t, J = 6.2 Hz, 1H), 5.57-5.45 (m, 1H), 5.44-5.34 (m, 1H), 5.04 (s, 2H), 4.43-4.35 |
| | (m, 2H), 4.21-4.15 (m, 2H), 4.11-4.07 (m, 2H), 4.00 (t, J = 4.5 Hz, 2H), 3.35 (s, 3H), 3.18 (d, |
| | J = 4.8 Hz, 1H), 3.13-2.99 (m, 4H), 1.72 (d, J = 12.2 Hz, 2H), 1.35 (s, 3H), 1.25-1.20 (m, 1H). |
| 114 | ¹H NMR (400 MHz, DMSO) δ 11.33 (s, 1H), 8.35 (d, J = 2.5 Hz, 1H), 7.94 (d, J = 8.7 Hz, 1H), |
| | 7.78-7.62 (m, 2H), 7.23 (d, J = 7.6 Hz, 2H), 7.06 (d, J = 2.5 Hz, 1H), 6.85 (d, J = 5.8 Hz, 1H), |
| | 6.02 (d, J = 15.8 Hz, 1H), 5.57 (d, J = 15.7 Hz, 1H), 5.02 (s, 2H), 4.14-4.07 (m, 4H), 4.01- |
| | 3.94 (m, 2H), 3.87 (s, 2H), 3.35 (s, 3H), 3.15 (d, J = 12.3 Hz, 2H), 2.92 (t, J = 11.8 Hz, 2H), 2.29 |
| | (d, J = 16.4 Hz, 1H), 1.67 (d, J = 12.2 Hz, 2H), 1.24 (s, 4H). |
| 115 | ¹H NMR (400 MHz, DMSO-d₆) δ 11.39 (s, 1 H) 10.13 (br d, J = 3.3 Hz, 1 H) 8.16 (d, J = 2.3 Hz, 1 |
| | H) 8.02 (d, J = 8.8 Hz, 1 H) 7.79 (d, J = 1.8 Hz, 1 H) 7.61-7.72 (m, 2 H) 7.23 (br t, J = 5.9 Hz, 1 H) |
| | 7.13 (d, J = 2.0 Hz, 1 H) 7.07 (dd, J = 8.6, 1.9 Hz, 1 H) 5.45-5.60 (m, 1 H) 5.31 (td, J = 10.5, 2.3 |
| | Hz, 1 H) 4.84-5.03 (m, 1 H) 4.60-4.79 (m, 1 H) 4.23 (t, J = 10.0 Hz, 1 H) 3.68-3.86 (m, 3 H) |
| | 3.50-3.65 (m, 1 H) 3.36 (t, J = 6.5 Hz, 2 H) 3.08-3.19 (m, 2 H) 2.83 (d, J = 11.5 Hz, 1 H) 2.39- |
| | 2.48 (m, 2 H) 2.17 (br d, J = 14.0 Hz, 1 H) 1.46-1.59 (m, 1 H) 1.30-1.44 (m, 2 H) 1.26 (s, 3 H) |
| 116 | ¹H NMR (400 MHz, DMSO-d₆) δ 10.04 (br s, 1 H) 9.67 (s, 1 H) 8.19 (d, J = 2.5 Hz, 1 H) 7.80 (d, |
| | J = 1.8 Hz, 1 H) 7.75 (d, J = 8.3 Hz, 1 H) 7.52-7.67 (m, 2 H) 7.19-7.24 (m, 1 H) 6.92-7.00 (m, 2 |
| | H) 6.52-6.58 (m, 1 H) 5.50-5.75 (m, 2 H) 4.93 (br d, J = 3.5 Hz, 1 H) 3.66-3.96 (m, 6 H) 3.34- |
| | 3.36 (m, 2 H) 3.03-3.19 (m, 2 H) 2.71 (br d, J = 11.3 Hz, 1 H) 2.41-2.50 (m, 1 H) 2.08 (br d, |
| | J = 13.3 Hz, 1 H) 1.61-1.79 (m, 1 H) 1.10-1.27 (m, 5 H) |
| 117 | ¹H NMR (400 MHz, DMSO-d₆) δ 11.40 (s, 1 H) 8.16 (d, J = 2.3 Hz, 1 H) 8.01 (d, J = 8.8 Hz, 1 H) |
| | 7.79 (d, J = 1.8 Hz, 1 H) 7.59-7.73 (m, 2 H) 7.23 (t, J = 6.0 Hz, 1 H) 7.11 (d, J = 1.3 Hz, 1 H) 7.06 |
| | (dd, J = 8.4, 1.6 Hz, 1 H) 6.54 (t, J = 2.1 Hz, 1 H) 5.47-5.57 (m, 1 H) 5.24-5.38 (m, 1 H) 4.61- |
| | 4.77 (m, 1 H) 4.23 (t, J = 9.6 Hz, 1 H) 3.69-3.85 (m, 3 H) 3.50-3.65 (m, 1 H) 3.33-3.38 (m, 2 |
| | H) 3.07-3.19 (m, 2 H) 2.83 (d, J = 11.5 Hz, 1 H) 2.52-2.58 (m, 2 H) 2.44 (br s, 2 H) 2.11-2.22 |
| | (m, 1 H) 1.45-1.57 (m, 1 H) 1.29-1.42 (m, 1 H) 1.26 (s, 3 H) |
| 118 | ¹H NMR (400 MHz, DMSO-d₆) δ 10.95 (s, 1 H) 10.21 (br s, 1 H) 8.76 (s, 1 H) 8.22 (s, 1 H) 7.89 |
| | (d, J = 8.5 Hz, 1 H) 7.58-7.71 (m, 2 H) 7.10 (d, J = 1.8 Hz, 1 H) 7.03 (dd, J = 8.6, 1.9 Hz, 1 H) 6.53 |
| | (t, J = 6.5 Hz, 1 H) 5.46-5.66 (m, 2 H) 3.58-3.68 (m, 2 H) 3.29 (s, 3 H) 3.16-3.26 (m, 4 H) 2.68 |
| | (br t, J = 11.4 Hz, 2 H) 2.17 (br d, J = 4.0 Hz, 2 H) 2.06 (br dd, J = 6.5, 3.5 Hz, 2 H) 1.73-1.90 (m, 2 |
| | H) 1.60-1.73 (m, 1 H) 1.54 (br d, J = 11.0 Hz, 2 H) 1.21 (t, J = 7.3 Hz, 3 H) |
| 119 | ¹H NMR (400 MHz, DMSO-d₆) δ 12.62 (s, 1 H) 10.27 (br s, 1 H) 8.75 (s, 1 H) 8.22 (s, 1 H) 8.12 |
| | (d, J = 8.8 Hz, 1 H) 7.66 (d, J = 8.5 Hz, 1 H) 7.38 (d, J = 8.3 Hz, 1 H) 7.25 (d, J = 1.8 Hz, 1 H) 7.15 |
| | (dd, J = 8.6, 2.1 Hz, 1 H) 6.76 (t, J = 6.3 Hz, 1 H) 5.43-5.59 (m, 2 H) 3.39-3.48 (m, 2 H) 3.29 (s, |
| | 3 H) 3.23 (q, J = 7.3 Hz, 2 H) 3.08-3.17 (m, 2 H) 2.78 (br t, J = 11.3 Hz, 2 H) 2.14-2.30 (m, 4 H) |
| | 1.66-2.00 (m, 5 H) 1.21 (t, J = 7.4 Hz, 3 H) |
| 120 | ¹H NMR (400 MHz, DMSO-d₆) δ 10.93 (s, 1 H) 8.55 (s, 1 H) 8.10 (s, 1 H) 7.89 (d, J = 8.5 Hz, 1 |
| | H) 7.58-7.70 (m, 2 H) 7.09 (d, J = 1.8 Hz, 1 H) 7.02 (dd, J = 8.5, 1.8 Hz, 1 H) 6.58-6.70 (m, 1 H) |
| | 5.43-5.67 (m, 2 H) 3.59-3.68 (m, 3 H) 3.17-3.25 (m, 4 H) 2.68 (br t, J = 11.3 Hz, 3 H) 2.11- |
| | 2.24 (m, 2 H) 2.06 (br dd, J = 6.9, 3.4 Hz, 2 H) 1.74-1.89 (m, 2 H) 1.59-1.72 (m, 1 H) 1.54 (br d, |
| | J = 10.8 Hz, 2 H) 1.14-1.27 (m, 3 H) |
| 121 | ¹H NMR (400 MHz, DMSO-d₆) δ 12.59 (s, 1 H) 8.57 (s, 1 H) 8.07-8.16 (m, 2 H) 7.67 (d, J = 8.0 |
| | Hz, 1 H) 7.35 (d, J = 8.5 Hz, 1 H) 7.20-7.29 (m, 1 H) 7.11-7.18 (m, 1 H) 6.78-6.89 (m, 1 H) |
| | 5.50 (br s, 2 H) 3.23 (m, J = 7.8, 3.6, 3.6 Hz, 3 H) 2.73-2.86 (m, 2 H) 2.12-2.30 (m, 6 H) 1.65- |
| | 1.98 (m, 7 H) 1.18-1.25 (m, 4 H) |
| 122 | ¹H NMR (400 MHz, DMSO-d₆) δ 10.93 (s, 1 H) 10.04-10.29 (m, 1 H) 8.19 (d, J = 2.3 Hz, 1 H) |
| | 7.79-8.01 (m, 2 H) 7.66 (s, 2 H) 7.38 (br s, 1 H) 7.07 (s, 1 H) 6.91-7.02 (m, 2 H) 6.86 (d, J = 2.5 |
| | Hz, 1 H) 5.42-5.66 (m, 2 H) 3.78-4.28 (m, 1 H) 3.69 (br s, 2 H) 3.08-3.26 (m, 5 H) 2.61-2.76 |
| | (m, 3 H) 2.11-2.26 (m, 2 H) 1.98-2.11 (m, 2 H) 1.72-1.88 (m, 2 H) 1.59-1.72 (m, 1 H) 1.48- |
| | 1.58 (m, 2 H) |
| 123 | ¹H NMR (400 MHz, DMSO-d₆) δ 11.85 (br s, 1 H) 8.17 (d, J = 2.5 Hz, 1 H) 8.06 (d, J = 8.8 Hz, 1 |
| | H) 7.79 (d, J = 1.5 Hz, 1 H) 7.66 (s, 2 H) 7.15-7.24 (m, 2 H) 7.08 (dd, J = 8.8, 2.0 Hz, 1 H) 6.51- |
| | 6.58 (m, 1 H) 4.66-4.79 (m, 1 H) 3.63-3.83 (m, 4 H) 3.53 (br d, J = 10.0 Hz, 1 H) 3.35 (s, 2 H) |
| | 3.11-3.20 (m, 3 H) 3.04 (br d, J = 10.5 Hz, 2 H) 2.38-2.49 (m, 3 H) 2.23-2.36 (m, 1 H) 1.99- |
| | 2.10 (m, 1 H) 1.41-1.58 (m, 2 H) 1.21-1.40 (m, 1 H) |
| 124 | ¹H NMR (400 MHz, DMSO-d₆) δ 11.87 (s, 1H), 8.16 (s, 1H), 8.03 (d, J = 8.8 Hz, 1H), 7.79 (d, J = |
| | 2.4 Hz, 1H), 7.66 (s, 2H), 7.17-7.15 (m, 2H), 7.06-7.04 (m, 1H), 6.55-6.54 (m, 1H), 4.73 |
| | (s, 1H), 3.78-3.67 (m, 5H), 3.58-3.46 (m, 2H), 3.21-3.04 (m, 4H), 3.03 (s, 2H), 2.33-2.30 |
| | (m, 2H), 2.06-2.04 (m, 1H), 1.49 (s, 3H), 1.32-1.24 (m, 2H). |
| 125 | ¹H NMR (400 MHz, DMSO-d₆) δ 11.86 (s, 1H), 8.16 (d, J = 2.4 Hz, 1H), 8.04 (d, J = 8.8 Hz, |
| | 1H), 7.79 (d, J = 2.4 Hz, 1H), 7.66 (s, 2H), 7.19-7.16 (m, 2H), 7.07-7.04 (m, 1H), 6.55-6.54 |
| | (m, 1H), 4.72 (s, 1H), 3.77-3.67 (m, 5H), 3.54-3.45 (m, 2H), 3.33-3.30 (m, 2H), 3.17-3.14 |
| | (m, 3H), 3.04-3.02 (m, 2H), 2.50-2.48 (m, 1H), 2.33-2.31 (m, 1H), 2.06-2.03 (m, 1H), 1.52- |
| | 1.49 (m, 2H), 1.32-1.23 (m, 2H). |
| 126 | ¹H NMR (400 MHz, DMSO-d₆) δ 12.25 (br s, 1 H) 10.22 (s, 1 H) 8.16 (d, J = 2.5 Hz, 1 H) 8.08 (d, |
| | J = 8.8 Hz, 1 H) 7.79 (d, J = 1.8 Hz, 1 H) 7.55-7.71 (m, 2 H) 7.24 (d, J = 2.0 Hz, 1 H) 7.11 (br dd, |
| | J = 8.5, 2.0 Hz, 2 H) 6.51-6.57 (m, 1 H) 4.98 (s, 1 H) 3.77 (t, J = 6.5 Hz, 2 H) 3.41-3.70 (m, 7 H) |
| | 3.33-3.38 (m, 3 H) 3.05-3.15 (m, 1 H) 2.99 (br s, 1 H) 2.83 (td, J = 4.1, 0.9 Hz, 1 H) 2.62-2.73 |
| | (m, 1 H) 1.94-2.08 (m, 1 H) 1.75-1.92 (m, 1 H) 1.58-1.74 (m, 2 H) 1.28-1.50 (m, 1 H) |
| 127 | ¹H NMR (400 MHz, DMSO-d₆) δ 12.27 (s, 1H), 8.16 (d, J = 2.4 Hz, 1H), 8.09 (d, J = 8.6 Hz, |
| | 1H), 7.79 (d, J = 1.9 Hz, 1H), 7.67 (d, J = 8.3 Hz, 1H), 7.59 (d, J = 8.3 Hz, 1H), 7.25 (d, J = 2.1 |
| | Hz, 1H), 7.19-7.05 (m, 2H), 6.54 (t, J = 2.2 Hz, 1H), 3.77 (t, J = 6.4 Hz, 2H), 3.68-3.35 (m, |
| | 10H), 3.11 (d, J = 10.3 Hz, 1H), 2.99 (s, 1H), 2.83 (s, 1H), 2.68 (s, 1H), 2.02 (s, 1H), 1.85 (s, 1H), |
| | 1.66 (s, 2H), 1.34 (s, 1H), 1.24 (s, 1H), 0.85 (d, J = 8.3 Hz, 1H). |
| 129 | ¹H NMR (400 MHz, DMSO) δ 11.79 (s, 1H), 8.15 (d, J = 2.4 Hz, 1H), 8.01 (dd, J = 8.8, 1.2 Hz, |
| | 1H), 7.79 (d, J = 2.0 Hz, 1H), 7.62 (d, J = 2.0 Hz, 2H), 7.23 (d, J = 2.8 Hz, 1H), 7.09-6.98 (m, |
| | 1H), 6.90-6.89 (m, 1H), 6.54-6.53 (m, 1H), 3.79-3.75 (m, 4H), 3.40-3.37 (m, 2H), 3.15 (s, |
| | 2H), 3.01-2.99 (m, 2H), 2.99 (s, 2H), 2.92-2.89 (m, 2H), 2.45 (s, 2H), 1.69-1.66 (d, J = 6.5 |
| | Hz, 2H), 1.26-1.23 (m, 2H), 1.19-1.09 (m, 2H), 0.92 (s, 3H). |
| 130 | ¹H NMR (400 MHz, DMSO-d6) δ ppm 11.70 (s, 1 H) 8.16 (d, J = 2.3 Hz, 1 H) 8.05 (d, J = 8.5 Hz, |
| | 1 H) 7.79 (d, J = 1.3 Hz, 1 H) 7.66 (s, 2 H) 7.18 (br s, 2 H) 7.09 (br d, J = 8.8 Hz, 1 H) 6.54 (t, J = 1.8 |
| | Hz, 1 H) 4.81 (br d, J = 8.3 Hz, 1 H) 3.76 (br t, J = 6.4 Hz, 4 H) 3.45 (br s, 2 H) 3.18 (br d, J = 7.5 |
| | Hz, 2 H) 2.86-3.07 (m, 2 H) 2.72-2.83 (m, 2 H) 2.40 (br d, J = 5.8 Hz, 2 H) 2.05 (br d, J = 12.5 |
| | Hz, 1 H) 1.53 (br s, 1 H) 0.85-1.35 (m, 8 H) |
| 131 | ¹H NMR (400 MHz, DMSO-d₆) δ ppm 11.69 (s, 1 H) 8.16 (d, J = 2.3 Hz, 1 H) 8.07 (d, J = 8.5 Hz, 1 |
| | H) 7.79 (d, J = 1.8 Hz, 1 H) 7.66 (s, 2 H) 7.15-7.24 (m, 2 H) 7.11 (dd, J = 8.8, 1.8 Hz, 1 H) 6.54 (t, |
| | J = 2.1 Hz, 1 H) 4.81 (d, J = 8.5 Hz, 1 H) 3.76 (br t, J = 6.5 Hz, 4 H) 3.47 (br d, J = 9.0 Hz, 1 H) 3.35- |
| | 3.38 (m, 3 H) 3.18 (br d, J = 7.0 Hz, 1 H) 2.87-3.06 (m, 2 H) 2.74-2.84 (m, 2 H) 2.52-2.59 (m, |
| | 2 H) 2.35-2.45 (m, 2 H) 2.05 (br d, J = 12.5 Hz, 1 H) 1.54 (br dd, J = 2.4, 0.9 Hz, 1 H) 1.16-1.33 |
| | (m, 1 H) 0.97-1.15 (m, 4 H) |
| 132 | ¹H NMR (400 MHz, DMSO-d₆) δ 11.69 (s, 1 H) 10.16 (s, 1 H) 8.16 (d, J = 2.5 Hz, 1 H) 8.07 (d, |
| | J = 8.5 Hz, 1 H) 7.79 (d, J = 1.8 Hz, 1 H) 7.66 (s, 2 H) 7.16-7.25 (m, 2 H) 7.12 (dd, J = 8.6, 1.9 Hz, |
| | 1 H) 6.54 (t, J = 2.1 Hz, 1 H) 4.94 (br t, J = 5.6 Hz, 1 H) 4.81 (d, J = 8.5 Hz, 1 H) 3.68-3.86 (m, 4 |
| | H) 3.43-3.52 (m, 2 H) 3.36-3.38 (m, 2 H) 3.13-3.23 (m, 1 H) 2.87-3.08 (m, 2 H) 2.73-2.85 |
| | (m, 2 H) 2.35-2.45 (m, 2 H) 2.05 (br d, J = 12.3 Hz, 1 H) 1.46-1.59 (m, 1 H) 1.18-1.33 (m, 1 |
| | H) 0.97-1.16 (m, 4 H) |
| 137 | ¹H NMR (400 MHz, DMSO-d6) δ 12.97 (s, 1H), 8.75 (d, J = 0.7 Hz, 1H), 8.22 (d, J = 0.7 Hz, |
| | 1H), 8.18 (d, J = 8.6 Hz, 1H), 7.67 (d, J = 8.4 Hz, 1H), 7.62 (d, J = 8.3 Hz, 1H), 7.45 (d, J = 2.2 |
| | Hz, 1H), 7.34 (dd, J = 8.7, 2.2 Hz, 1H), 6.76 (s, 1H), 5.57-5.47 (m, 1H), 5.45-5.32 (m, 1H), |
| | 5.03 (s, 2H), 4.38 (s, 2H), 4.18 (d, J = 4.6 Hz, 2H), 4.09 (dd, J = 5.6, 3.5 Hz, 2H), 4.03-3.91 (m, |
| | 2H), 3.29 (s, 3H), 3.14-2.97 (m, 4H), 2.58-2.53 (m, 2H), 1.71 (d, J = 12.3 Hz, 2H), 1.35 (s, |
| | 3H). |
| 138 | 1H NMR (400 MHz, DMSO-d6) δ 11.28 (s, 1H), 8.80 (s, 1H), 8.22 (s, 1H), 7.93 (d, J = 9.1 Hz, |
| | 1H), 7.71 (d, J = 8.3 Hz, 1H), 7.63 (d, J = 8.3 Hz, 1H), 7.27-7.12 (m, 2H), 6.92 (t, J = 5.3 Hz, |
| | 1H), 5.99 (d, J = 16.0 Hz, 1H), 5.56 (d, J = 15.8 Hz, 1H), 5.02 (s, 2H), 4.10 (dd, J = 11.0, 6.6 Hz, |
| | 4H), 3.97 (t, J = 4.6 Hz, 2H), 3.87 (s, 2H), 3.29 (s, 3H), 3.15 (d, J = 11.8 Hz, 2H), 2.91 (t, J = 12.0 |
| | Hz, 2H), 2.32-2.20 (m, 2H), 1.66 (d, J = 12.2 Hz, 2H), 1.24 (s, 3H). |
| 139 | 1H NMR (400 MHz, DMSO) δ 12.98 (s, 1H), 8.32 (d, J = 2.5 Hz, 1H), 8.18 (d, J = 8.6 Hz, 1H), |
| | 7.71-7.56 (m, 2H), 7.45 (d, J = 2.2 Hz, 1H), 7.34 (dd, J = 8.6, 2.1 Hz, 1H), 7.25 (d, J = 2.5 Hz, |
| | 1H), 6.68 (t, J = 6.2 Hz, 1H), 5.56-5.44 (m, 1H), 5.38-5.33 (m, 1H), 5.03 (s, 2H), 4.36 (s, 2H), |
| | 4.23-4.15 (m, 2H), 4.09 (dd, J = 5.7, 3.3 Hz, 2H), 4.00 (dd, J = 5.6, 3.3 Hz, 2H), 3.05-3.02 (m, |
| | 4H), 2.68-2.65 (m 1H), 2.08 (s, 1H), 1.70 (d, J = 12.3 Hz, 2H), 1.35 (s, 3H). |
| 140 | 1H NMR (400 MHz, DMSO) δ 12.98 (s, 1H), 8.32 (d, J = 2.5 Hz, 1H), 8.18 (d, J = 8.6 Hz, 1H), |
| | 7.71-7.56 (m, 2H), 7.45 (d, J = 2.2 Hz, 1H), 7.34 (dd, J = 8.6, 2.1 Hz, 1H), 7.25 (d, J = 2.5 Hz, |
| | 1H), 6.68 (t, J = 6.2 Hz, 1H), 5.56-5.44 (m, 1H), 5.38-5.33 (m, 1H), 5.03 (s, 2H), 4.36 (s, 2H), |
| | 4.23-4.15 (m, 2H), 4.09 (dd, J = 5.7, 3.3 Hz, 2H), 4.00 (dd, J = 5.6, 3.3 Hz, 2H), 3.05-3.02 (m, |
| | 4H), 2.68-2.65 (m 1H), 2.08 (s, 1H), 1.70 (d, J = 12.3 Hz, 2H), 1.35 (s, 3H). |
| 141 | 1H NMR (400 MHz, DMSO-d6) δ 10.70 (s, 1H), 8.19 (s, 1H), 8.07 (d, J = 8.4 Hz, 1H), 7.82 (s, |
| | 1H), 7.67 (s, 2H), 7.49 (dd, J = 8.0 Hz, 1.6 Hz, 1H), 7.38 (s, 1H), 7.04-7.00 (m, 1H), 6.57-6.56 |
| | (m, 1H), 5.68-5.45 (m, 2H), 3.71-3.64 (m, 6H), 3.32-3.27 (m, 2H), 2.94-2.92 (m, 4H), 2.81 (t, J = |
| | 11.6 Hz, 2H), 2.22-2.15 (m, 2H), 2.07-2.04 (m, 2H), 0.88-0.72 (m, 2H), 0.71-0.59 (m, 1H), 0.57- |
| | 0.50 (m, 2H). |
| 142 | 1H NMR (400 MHz, DMSO-d6) δ 12.53 (s, 1H), 8.40 (d, J = 8.8 Hz, 1H), 8.20 (s, 1H), 7.82 (s, |
| | 1H), 7.71-7.67 (m, 3H), 7.36 (d, J = 8.4 Hz, 1H), 7.28-7.24 (m, 1H), 6.57 (s, 1H), 5.53-5.49 (m, |
| | 2H), 3.66-3.64 (m, 4H), 3.49-3.47 (m, 2H), 3.22-3.18 (m, 2H), 3.00-2.93 (m, 6H), 2.34-2.27 (m, |
| | 2H), 2.21-2.18 (m, 2H), 2.08-1.97 (m, 2H), 1.84-1.81 (m, 2H), 1.80-1.69 (m, 1H). |
| 143 | 1H NMR (400 MHz, DMSO-d6) δ 10.76 (s, 1H), 8.78 (d, J = 0.7 Hz, 1H), 8.23 (d, J = 0.7 Hz, |
| | 1H), 8.07 (d, J = 8.0 Hz, 1H), 7.73-7.61 (m, 4H), 6.59 (t, J = 6.6 Hz, 1H), 5.54 (ddt, J = 28.5, |
| | 15.4, 7.2 Hz, 2H), 3.65 (s, 2H), 3.30 (s, 3H), 3.29 (s, 3H), 2.83 (t, J = 11.6 Hz, 2H), 2.56-2.52 |
| | (m, 2H), 2.20-2.12 (m, 2H), 2.09-2.02 (m, 2H), 1.87-1.73 (m, 2H), 1.72-1.59 (m, 1H), 1.58- |
| | 1.47 (m, 2H). |
| 144 | 1H NMR (400 MHz, DMSO-d6) δ 12.66 (s, 1H), 8.77 (s, 1H), 8.38 (d, J = 8.3 Hz, 1H), 8.24 (s, |
| | 1H), 7.96 (d, J = 1.8 Hz, 1H), 7.88 (dd, J = 8.3, 1.8 Hz, 1H), 7.70 (d, J = 8.2 Hz, 1H), 7.38 (d, J = |
| | 8.2 Hz, 1H), 6.83 (t, J = 6.4 Hz, 1H), 5.61-5.29 (m, 2H), 3.44 (dt, J = 11.6, 6.6 Hz, 2H), 3.32 (s, |
| | 3H), 3.30 (s, 3H), 3.20-3.14 (m, 2H), 3.05-2.92 (m, 2H), 2.30-2.22 (m, 2H), 2.19 (t, J = 7.2 |
| | Hz, 2H), 1.99-1.87 (m, 2H), 1.86-1.72 (m, 3H). |
| 145 | 1H NMR (400 MHz, DMSO) δ 10.75 (s, 1H), 8.78 (s, 1H), 8.23 (s, 1H), 8.07 (d, J = 8.1 Hz, 1H), |
| | 7.70-7.57 (m, 4H), 6.59 (t, J = 6.6 Hz, 1H), 5.54 (ddt, J = 28.4, 15.3, 7.1 Hz, 2H), 3.66 (s, 2H), |
| | 3.38 (q, J = 7.3 Hz, 2H), 3.30 (s, 4H), 2.82 (t, J = 11.6 Hz, 2H), 2.32 (d, J = 8.2 Hz, 1H), 2.17 (s, |
| | 2H), 2.06 (dd, J = 8.4, 4.3 Hz, 2H), 1.80 (q, J = 12.2 Hz, 2H), 1.68 (d, J = 12.1 Hz, 1H), 1.53 (d, J = |
| | 12.2 Hz, 2H), 1.14 (t, J = 7.3 Hz, 3H). |
| 146 | 1H NMR (400 MHz, DMSO) δ 12.65 (s, 1H), 8.78 (s, 1H), 8.39 (d, J = 8.2 Hz, 1H), 8.24 (s, 1H), |
| | 7.90 (d, J = 1.7 Hz, 1H), 7.83 (dd, J = 8.2, 1.6 Hz, 1H), 7.70 (d, J = 8.2 Hz, 1H), 7.42-7.35 (m, |
| | 1H), 6.83 (t, J = 6.4 Hz, 1H), 5.50 (d, J = 6.1 Hz, 2H), 3.40 (q, J = 7.3 Hz, 5H), 3.30 (s, 3H), 3.18 |
| | (d, J = 11.2 Hz, 2H), 3.02-2.93 (m, 2H), 2.29-2.15 (m, 4H), 1.93 (q, J = 11.1 Hz, 2H), 1.82 (d, |
| | J = 13.4 Hz, 2H), 1.14 (t, J = 7.3 Hz, 3H). |
| 147 | 1H NMR (400 MHz, DMSO-d6) δ 10.70 (s, 1H), 8.07 (dd, J = 4.4, 3.6 Hz, 2H), 7.75 (s, 1H), 7.72- |
| | 7.60 (m, 4H), 7.09 (t, J = 6.7 Hz, 1H), 5.67-5.43 (m, 2H), 5.01 (t, J = 5.4 Hz, 1H), 4.47 (d, J = |
| | 5.4 Hz, 2H), 3.70 (q, J = 6.0 Hz, 2H), 3.32-3.23 (m, 5H), 2.82 (t, J = 11.7 Hz, 2H), 2.21-2.13 |
| | (m, 2H), 2.06 (dd, J = 7.8, 3.6 Hz, 2H), 1.90-1.73 (m, 2H), 1.67 (d, J = 12.4 Hz, 1H), 1.53 (d, J = |
| | 12.2 Hz, 2H). |
| 148 | ¹H NMR (400 MHz, DMSO-d₆) δ 12.55 (s, 1 H) 8.38 (d, J = 8.3 Hz, 1 H) 8.07 (s, 1 H) 7.95 (d, |
| | J = 2.0 Hz, 1 H) 7.87 (dd, J = 8.3, 1.8 Hz, 1 H) 7.74 (s, 1 H) 7.67 (d, J = 8.3 Hz, 1 H) 7.35 (d, J = 8.3 |
| | Hz, 1 H) 7.31 (t, J = 6.4 Hz, 1 H) 5.44-5.57 (m, 2 H) 5.01 (t, J = 5.4 Hz, 1 H) 4.42-4.51 (m, 2 H) |
| | 3.42-3.55 (m, 2 H) 3.28-3.38 (m, 3 H) 3.12-3.23 (m, 2 H) 2.91-3.03 (m, 2 H) 2.23-2.33 (m, |
| | 2 H) 2.14-2.22 (m, 2 H) 1.88-2.03 (m, 2 H) 1.67-1.87 (m, 3 H). |
| 149 | ¹H NMR (400 MHz, DMSO-d₆) δ ppm 10.69 (s, 1 H) 8.02-8.10 (m, 2 H) 7.75 (s, 1 H) 7.61- |
| | 7.68 (m, 3 H) 7.60 (d, J = 1.5 Hz, 1 H) 7.09 (t, J = 6.6 Hz, 1 H) 5.44-5.66 (m, 2 H) 5.00 (t, J = 5.4 |
| | Hz, 1 H) 4.47 (d, J = 5.3 Hz, 2 H) 3.63-3.78 (m, 2 H) 3.24-3.43 (m, 4 H) 2.81 (br t, J = 11.6 Hz, 2 |
| | H) 2.13-2.25 (m, 2 H) 2.06 (br dd, J = 7.1, 3.1 Hz, 2 H) 1.73-1.88 (m, 2 H) 1.60-1.72 (m, 1 H) |
| | 1.47-1.57 (m, 2 H) 1.14 (t, J = 7.4 Hz, 3 H). |
| 150 | ¹H NMR (400 MHz, DMSO-d₆) δ 12.54 (s, 1 H) 8.36-8.42 (m, 1 H) 8.07 (s, 1 H) 7.89 (s, 1 H) |
| | 7.82 (dd, J = 8.1, 1.6 Hz, 1 H) 7.74 (s, 1 H) 7.67 (d, J = 8.3 Hz, 1 H) 7.27-7.39 (m, 2 H) 5.44-5.59 |
| | (m, 2 H) 4.98-5.05 (m, 1 H) 4.47 (d, J = 5.3 Hz, 2 H) 3.36-3.53 (m, 4 H) 3.18 (br d, J = 10.3 Hz, |
| | 2 H) 2.97 (br t, J = 11.4 Hz, 2 H) 2.24-2.32 (m, 2 H) 2.19 (br t, J = 6.3 Hz, 2 H) 1.88-2.02 (m, 2 |
| | H) 1.69-1.87 (m, 3 H) 1.14 (t, J = 7.4 Hz, 3 H). |
| 151 | ¹H NMR (400 MHz, DMSO-d₆) δ 12.97 (s, 1 H) 8.90 (s, 1 H) 8.37 (s, 1 H) 8.17 (d, J = 8.8 Hz, 1 |
| | H) 7.58-7.66 (m, 2 H) 7.45 (d, J = 2.3 Hz, 1 H) 7.34 (dd, J = 8.6, 2.1 Hz, 1 H) 6.65 (t, J = 6.1 Hz, 1 |
| | H) 5.33-5.54 (m, 2 H) 5.03 (s, 2 H) 4.35 (br t, J = 4.9 Hz, 2 H) 4.17 (br d, J = 3.8 Hz, 2 H) 3.95- |
| | 4.13 (m, 5 H) 2.97-3.15 (m, 5 H) 1.70 (br d, J = 12.0 Hz, 2 H) 1.35 (s, 3 H). |
| 152 | ¹H NMR (400 MHz, DMSO-d₆) δ 11.29 (s, 1 H) 8.95 (s, 1 H) 8.38 (s, 1 H) 7.93 (d, J = 9.0 Hz, 1 |
| | H) 7.60-7.71 (m, 2 H) 7.18-7.27 (m, 2 H) 6.78-6.88 (m, 1 H) 5.92-6.05 (m, 1 H) 5.55 (br d, |
| | J = 16.5 Hz, 1 H) 5.02 (s, 2 H) 4.10 (dt, J = 8.8, 4.1 Hz, 4 H) 3.93-4.01 (m, 2 H) 3.84 (br d, J = 1.5 |
| | Hz, 2 H) 3.09-3.20 (m, 2 H) 2.91 (br t, J = 11.6 Hz, 2 H) 2.20-2.30 (m, 2 H) 1.66 (br d, J = 11.5 |
| | Hz, 2 H) 1.24 (s, 3 H). |
| 153 | ¹H NMR (400 MHz, DMSO-d₆) δ 12.67 (s, 1 H) 8.35 (d, J = 8.3 Hz, 1 H) 8.18 (d, J = 2.3 Hz, 1 H) |
| | 8.03 (d, J = 1.8 Hz, 1 H) 7.85 (dd, J = 8.3, 1.8 Hz, 1 H) 7.81 (d, J = 2.0 Hz, 1 H) 7.70 (d, J = 8.3 Hz, 1 |
| | H) 7.60 (d, J = 8.3 Hz, 1 H) 7.23 (t, J = 6.4 Hz, 1 H) 6.53-6.59 (m, 1 H) 5.32-5.58 (m, 2 H) 4.40 |
| | (br t, J = 5.6 Hz, 2 H) 4.18 (br d, J = 3.8 Hz, 2 H) 3.33 (s, 3 H) 3.04-3.21 (m, 4 H) 2.52-2.58 (m, |
| | 2 H) 1.70 (br d, J = 12.3 Hz, 2 H) 1.35 (s, 3 H). |
| 154 | 1H NMR (400 MHz, DMSO) δ 10.90 (s, 1H), 8.22 (dd, J = 2.4, 0.6 Hz, 1H), 8.02 (d, J = 8.0 Hz, |
| | 1H), 7.80 (d, J = 1.9 Hz, 1H), 7.76-7.65 (m, 3H), 7.61 (d, J = 8.3 Hz, 1H), 7.33 (t, J = 5.4 Hz, |
| | 1H), 6.61-6.52 (m, 1H), 5.99 (d, J = 15.9 Hz, 1H), 5.54 (d, J = 15.7 Hz, 1H), 4.10 (s, 2H), 3.92 |
| | (s, 2H), 3.28 (s, 5H), 2.96 (s, 2H), 2.33-2.13 (m, 2H), 1.63 (d, J = 12.4 Hz, 2H), 1.22 (s, 3H). |
| 155 | 1H NMR (400 MHz, DMSO) δ 12.66 (s, 1H), 8.35 (dd, J = 8.1, 1.8 Hz, 1H), 8.18 (t, J = 2.1 Hz, |
| | 1H), 7.97 (d, J = 1.9 Hz, 1H), 7.85-7.77 (m, 2H), 7.70 (dd, J = 8.2, 1.8 Hz, 1H), 7.60 (dd, J = |
| | 8.2, 1.8 Hz, 1H), 7.22 (t, J = 6.0 Hz, 1H), 6.56 (q, J = 2.1 Hz, 1H), 5.55-5.34 (m, 2H), 4.40 (t, J = |
| | 6.3 Hz, 2H), 4.19 (d, J = 4.7 Hz, 2H), 3.45-3.37 (m, 3H), 3.18 (d, J = 11.1 Hz, 2H), 3.09 (t, J = |
| | 12.1 Hz, 2H), 1.70 (d, J = 12.4 Hz, 2H), 1.36 (d, J = 1.8 Hz, 3H), 1.15 (td, J = 7.3, 1.9 Hz, 4H). |
| 156 | 1H NMR (400 MHz, DMSO) δ 10.88 (s, 1H), 8.22 (d, J = 2.4 Hz, 1H), 8.05-7.98 (m, 1H), 7.84- |
| | 7.79 (m, 1H), 7.77-7.58 (m, 4H), 7.33 (t, J = 5.5 Hz, 1H), 6.59-6.54 (m, 1H), 5.99 (d, J = |
| | 15.7 Hz, 1H), 5.54 (d, J = 15.7 Hz, 1H), 4.13-4.07 (m, 2H), 3.92 (s, 2H), 3.37 (q, J = 7.6 Hz, |
| | 3H), 3.26 (d, J = 12.1 Hz, 1H), 2.99 (t, J = 11.5 Hz, 2H), 2.25-2.16 (m, 2H), 1.63 (d, J = 12.3 |
| | Hz, 2H), 1.22 (s, 3H), 1.14 (t, J = 7.3 Hz, 3H). |
| 157 | 1H NMR (400 MHz, DMSO-d6) δ 10.96 (s, 1H), 10.15 (s, 1H), 8.23 (s, 1H), 7.89 (d, J = 8.6 Hz, |
| | 1H), 7.65 (d, J = 8.4 Hz, 1H), 7.58 (d, J = 8.3 Hz, 1H), 7.13-6.92 (m, 2H), 6.46 (t, J = 6.6 Hz, |
| | 1H), 5.74-5.45 (m, 2H), 5.39 (t, J = 5.2 Hz, 1H), 4.94 (s, 1H), 4.54 (d, J = 5.2 Hz, 2H), 3.76 (t, J = |
| | 6.5 Hz, 2H), 3.69-3.58 (m, 2H), 3.38-3.34 (m, 2H), 3.19 (d, J = 11.1 Hz, 2H), 2.68 (t, J = |
| | 11.5 Hz, 2H), 2.16 (d, J = 5.6 Hz, 2H), 2.06 (dd, J = 7.3, 3.7 Hz, 2H), 1.79 (dd, J = 13.9, 10.3 Hz, |
| | 2H), 1.68 (d, J = 12.2 Hz, 1H), 1.54 (d, J = 12.2 Hz, 2H). |
| 158 | 1H NMR (400 MHz, DMSO-d6) δ 12.64 (s, 1H), 10.21 (s, 1H), 8.23 (s, 1H), 8.11 (d, J = 8.7 Hz, |
| | 1H), 7.62 (d, J = 8.3 Hz, 1H), 7.38 (d, J = 8.3 Hz, 1H), 7.24 (d, J = 2.2 Hz, 1H), 7.14 (dd, J = 8.7, |
| | 2.1 Hz, 1H), 6.72 (t, J = 6.4 Hz, 1H), 5.55-5.45 (m, 2H), 5.40 (t, J = 5.2 Hz, 1H), 4.94 (s, |
| | 1H), 4.54 (d, J = 5.2 Hz, 2H), 3.76 (t, J = 6.5 Hz, 2H), 3.42-3.36 (m, 4H), 3.11 (d, J = 11.1 Hz, |
| | 2H), 2.79 (dd, J = 12.8, 10.1 Hz, 2H), 2.30-2.14 (m, 4H), 1.98-1.67 (m, J = 33.2, 15.8 Hz, |
| | 5H). |
| 159 | 1H NMR (400 MHz, DMSO-d6) δ 12.99 (s, 1H), 8.57-8.23 (m, 2H), 7.90 (d, J = 8.7 Hz, 1H), |
| | 7.72-7.49 (m, 2H), 7.23 (d, J = 2.4 Hz, 1H), 7.02-6.80 (m, 2H), 6.58 (t, J = 6.1 Hz, 1H), 5.58- |
| | 5.29 (m, 2H), 4.34 (d, J = 6.6 Hz, 2H), 4.15 (d, J = 4.6 Hz, 2H), 3.72 (t, J = 6.6 Hz, 2H), 3.05 (dt, |
| | J = 10.7, 4.7 Hz, 6H), 2.86 (t, J = 12.2 Hz, 3H), 1.68 (d, J = 12.2 Hz, 2H), 1.31 (s, 3H). |
| 161 | ¹H NMR (400 MHz, DMSO-d₆) δ 12.72 (s, 1 H) 10.23 (br s, 1 H) 8.90 (s, 1 H) 8.37 (s, 1 H) 8.09 |
| | (d, J = 8.5 Hz, 1 H) 7.61 (s, 2 H) 7.28 (d, J = 2.0 Hz, 1 H) 7.13 (dd, J = 8.5, 2.0 Hz, 1 H) 6.63 (t, |
| | J = 6.3 Hz, 1 H) 5.31-5.55 (m, 2 H) 4.95 (br s, 1 H) 4.33 (br t, J = 5.3 Hz, 2 H) 4.17 (br d, J = 3.8 |
| | Hz, 2 H) 3.77 (t, J = 6.4 Hz, 2 H) 3.37 (t, J = 6.5 Hz, 2 H) 3.12 (br d, J = 11.3 Hz, 2 H) 2.88 (br t, |
| | J = 11.9 Hz, 2 H) 2.52-2.56 (m, 2 H) 1.70 (br d, J = 12.3 Hz, 2 H) 1.32 (s, 3 H). |
| 162 | ¹H NMR (400 MHz, DMSO-d₆) δ 11.24 (s, 1 H) 10.18 (br s, 1 H) 8.95 (s, 1 H) 8.37 (s, 1 H) 7.87 |
| | (d, J = 8.5 Hz, 1 H) 7.58-7.71 (m, 2 H) 7.13 (s, 1 H) 7.03 (dd, J = 8.6, 1.9 Hz, 1 H) 6.81 (t, J = 5.3 |
| | Hz, 1 H) 5.98 (br d, J = 15.8 Hz, 1 H) 5.50-5.64 (m, 1 H) 4.94 (br s, 1 H) 4.12 (br d, J = 1.8 Hz, 2 |
| | H) 3.83 (br s, 2 H) 3.76 (t, J = 6.6 Hz, 2 H) 3.33-3.37 (m, 2 H) 3.15 (br d, J = 11.5 Hz, 2 H) 2.81 |
| | (br t, J = 11.5 Hz, 2 H) 2.21-2.31 (m, 2 H) 1.66 (br d, J = 12.3 Hz, 2 H) 1.22 (s, 3 H). |
| 163 | 1H NMR (400 MHz, DMSO) δ 12.69 (s, 1H), 10.26 (s, 1H), 8.74 (s, 1H), 8.22 (s, 1H), 8.09 (d, J = |
| | 8.7 Hz, 1H), 7.63 (q, J = 8.4 Hz, 2H), 7.28 (d, J = 2.2 Hz, 1H), 7.14 (dd, J = 8.6, 2.0 Hz, 1H), |
| | 6.74 (t, J = 6.2 Hz, 1H), 5.51 (d, J = 11.0 Hz, 1H), 5.38 (dt, J = 11.9, 5.9 Hz, 1H), 4.35 (t, J = 5.8 |
| | Hz, 2H), 4.17 (d, J = 4.8 Hz, 2H), 3.29 (s, 3H), 3.23 (q, J = 7.3 Hz, 2H), 3.13 (d, J = 11.4 Hz, |
| | 2H), 2.88 (t, J = 12.1 Hz, 2H), 2.68 (s, 1H), 1.70 (d, J = 12.3 Hz, 2H), 1.32 (s, 3H), 1.22 (t, J = 7.3 |
| | Hz, 4H). |
| 164 | 1H NMR (400 MHz, DMSO) δ 11.21 (s, 1H), 10.19 (s, 1H), 8.79 (s, 1H), 8.22 (s, 1H), 7.87 (d, J = |
| | 8.6 Hz, 1H), 7.69 (d, J = 8.3 Hz, 1H), 7.63 (d, J = 8.4 Hz, 1H), 7.15 (d, J = 2.1 Hz, 1H), 7.04 |
| | (dd, J = 8.5, 2.0 Hz, 1H), 6.90 (t, J = 5.3 Hz, 1H), 5.98 (d, J = 15.8 Hz, 1H), 5.56 (d, J = 15.8 Hz, |
| | 1H), 4.12 (s, 2H), 3.86 (s, 2H), 3.29 (s, 3H), 3.22 (q, J = 7.3 Hz, 2H), 3.15 (d, J = 11.6 Hz, 2H), |
| | 2.87-2.73 (m, 2H), 2.29 (td, J = 13.5, 7.8 Hz, 2H), 1.66 (d, J = 12.1 Hz, 2H), 1.27-1.16 (m, |
| | 6H). |
| 165 | ¹H NMR (400 MHz, DMSO-d₆) δ 12.62 (s, 1 H) 8.02-8.13 (m, 2 H) 7.55-7.68 (m, 2 H) 7.20- |
| | 7.32 (m, 2 H) 7.07-7.18 (m, 1 H) 6.42-6.50 (m, 1 H) 5.34-5.56 (m, 2 H) 5.15 (t, J = 5.9 Hz, 1 |
| | H) 4.53 (d, J = 5.8 Hz, 2 H) 4.36-4.44 (m, 2 H) 4.18 (br d, J = 3.3 Hz, 2 H) 3.21 (q, J = 7.2 Hz, 2 H) |
| | 3.12 (br d, J = 10.5 Hz, 2 H) 2.87 (br t, J = 12.3 Hz, 2 H) 1.64-1.76 (m, 2 H) 1.32 (s, 4 H) 1.15- |
| | 1.26 (m, 5 H). |
| 166 | ¹H NMR (400 MHz, DMSO-d₆) δ 11.17 (s, 1 H) 8.10 (d, J = 2.3 Hz, 1 H) 7.86 (d, J = 8.5 Hz, 1 H) |
| | 7.57-7.71 (m, 2 H) 7.35 (t, J = 5.3 Hz, 1 H) 7.13 (s, 1 H) 7.02 (br d, J = 8.5 Hz, 1 H) 6.47 (d, J = 2.3 |
| | Hz, 1 H) 6.00 (br d, J = 16.0 Hz, 1 H) 5.48-5.60 (m, 1 H) 5.13 (t, J = 5.9 Hz, 1 H) 4.53 (d, J = 5.8 |
| | Hz, 2 H) 4.13 (br d, J = 2.0 Hz, 2 H) 3.91 (br d, J = 1.3 Hz, 2 H) 3.09-3.26 (m, 4 H) 2.73-2.87 (m, |
| | 2 H) 2.21-2.32 (m, 2 H) 1.65 (br d, J = 11.5 Hz, 2 H) 1.13-1.28 (m, 7 H). |
| 167 | 1H NMR (400 MHz, DMSO) δ 12.60 (s, 1H), 10.25 (s, 1H), 8.08 (d, J = 8.7 Hz, 1H), 8.03 (s, |
| | 1H), 7.72 (s, 1H), 7.66-7.56 (m, 2H), 7.26 (d, J = 2.1 Hz, 1H), 7.20 (t, J = 6.3 Hz, 1H), 7.12 (dd, |
| | J = 8.7, 2.1 Hz, 1H), 5.51 (d, J = 11.3 Hz, 1H), 5.37 (dt, J = 11.4, 5.8 Hz, 1H), 4.99 (t, J = 5.4 Hz, |
| | 1H), 4.46 (d, J = 5.3 Hz, 2H), 4.39 (s, 2H), 4.17 (d, J = 4.7 Hz, 2H), 3.22 (q, J = 7.2 Hz, 2H), 3.12 |
| | (d, J = 11.5 Hz, 2H), 2.87 (t, J = 12.2 Hz, 2H), 2.33 (d, J = 5.7 Hz, 1H), 1.69 (d, J = 12.3 Hz, 2H), |
| | 1.31 (s, 3H), 1.21 (t, J = 7.3 Hz, 4H). |
| 168 | 1H NMR (400 MHz, DMSO) δ 11.22 (s, 1H), 10.20 (s, 1H), 8.07 (s, 1H), 7.83 (d, J = 8.5 Hz, |
| | 1H), 7.72 (s, 1H), 7.64 (s, 2H), 7.30 (t, J = 5.4 Hz, 1H), 7.07 (s, 1H), 6.98 (d, J = 8.5 Hz, 1H), |
| | 5.99 (d, J = 15.7 Hz, 1H), 5.54 (d, J = 15.8 Hz, 1H), 4.99 (t, J = 5.4 Hz, 1H), 4.46 (d, J = 5.1 Hz, |
| | 2H), 4.12 (s, 2H), 3.90 (s, 2H), 3.17-3.09 (m, 4H), 2.79 (t, J = 12.1 Hz, 2H), 2.35-2.25 (m, |
| | 2H), 1.65 (d, J = 12.1 Hz, 2H), 1.23-1.15 (m, 6H). |
| 169 | ¹H NMR (400 MHz, DMSO) δ 12.63 (s, 1H), 10.24 (s, 1H), 8.16 (s, 1H), 8.06 (d, J = 8.7 Hz, |
| | 1H), 7.80 (d, J = 1.9 Hz, 1H), 7.63 (q, J = 8.3 Hz, 2H), 7.23 (s, 1H), 7.18-6.99 (m, 2H), 6.55 (d, |
| | J = 2.2 Hz, 1H), 5.50 (s, 1H), 5.37 (s, 1H), 4.39 (s, 2H), 4.17 (s, 2H), 3.20-3.02 (m, 4H), 2.87 (t, |
| | J = 12.2 Hz, 2H), 2.56-2.52 (m, 2H), 1.70 (d, J = 11.9 Hz, 2H), 1.32 (s, 3H), 1.21 (t, J = 7.3 Hz, |
| | 3H). |
| 170 | 1H NMR (400 MHz, DMSO-d6) δ 11.17 (s, 1H), 10.18 (s, 1H), 8.20 (d, J = 2.4 Hz, 1H), 7.86 (d, |
| | J = 8.5 Hz, 1H), 7.80 (d, J = 1.8 Hz, 1H), 7.69 (d, J = 8.3 Hz, 1H), 7.63 (d, J = 8.3 Hz, 1H), 7.26 |
| | (d, J = 5.5 Hz, 1H), 7.12 (s, 1H), 7.05-6.98 (m, 1H), 6.62-6.38 (m, 1H), 6.00 (d, J = 15.9 Hz, |
| | 1H), 5.54 (d, J = 15.5 Hz, 1H), 4.12 (s, 2H), 3.91 (s, 2H), 3.17 (dd, J = 18.1, 10.2 Hz, 4H), 2.80 (t, |
| | J = 12.1 Hz, 2H), 2.30 (d, J = 15.9 Hz, 2H), 1.65 (d, J = 12.2 Hz, 2H), 1.26-1.14 (m, 6H). |
| 171 | 1H NMR (400 MHz, DMSO-d6) δ 12.86 (s, 1H), 8.30-8.07 (m, 2H), 7.80 (d, J = 1.9 Hz, 1H), |
| | 7.75-7.56 (m, 2H), 7.43 (d, J = 2.2 Hz, 1H), 7.33 (dd, J = 8.6, 2.2 Hz, 1H), 7.18 (t, J = 6.3 Hz, |
| | 1H), 6.70-6.36 (m, 1H), 5.56-5.35 (m, 2H), 5.03 (s, 2H), 4.41 (s, 2H), 4.17 (d, J = 4.7 Hz, 2H), |
| | 4.08 (d, J = 4.9 Hz, 2H), 4.08-3.87 (m, 2H), 3.20-2.85 (m, 4H), 2.58-2.52 (m, 2H), 1.70 (d, J = |
| | 12.4 Hz, 2H), 1.34 (s, 3H). |
| 172 | 1H NMR (400 MHz, DMSO-d6) δ 11.21 (s, 1H), 8.21 (d, J = 2.5 Hz, 1H), 7.93 (d, J = 9.1 Hz, |
| | 1H), 7.80 (d, J = 1.9 Hz, 1H), 7.75-7.60 (m, 2H), 7.36-7.13 (m, 3H), 6.55 (s, 1H), 6.00 (d, J = |
| | 15.9 Hz, 1H), 5.54 (d, J = 15.9 Hz, 1H), 5.01 (s, 2H), 4.09 (dd, J = 10.7, 6.4 Hz, 4H), 4.02-3.86 |
| | (m, 4H), 3.14 (d, J = 11.8 Hz, 2H), 2.90 (t, J = 11.9 Hz, 2H), 2.28 (dd, J = 22.7, 10.0 Hz, 2H), |
| | 1.65 (d, J = 12.2 Hz, 2H), 1.23 (s, 3H). |
| 173 | 1H NMR (400 MHz, DMSO) δ 12.75 (s, 1H), 8.91 (s, 1H), 8.40-8.32 (m, 2H), 7.98 (s, 1H), |
| | 7.81 (d, J = 8.3 Hz, 1H), 7.68-7.55 (m, 2H), 6.69 (s, 1H), 5.50 (d, J = 11.7 Hz, 1H), 5.37 (s, 1H), |
| | 4.33 (s, 2H), 4.18 (d, J = 4.6 Hz, 2H), 3.41 (q, J = 7.3 Hz, 2H), 3.11 (dd, J = 26.9, 14.8 Hz, 4H), |
| | 2.33 (d, J = 8.1 Hz, 1H), 2.08 (s, 1H), 1.69 (d, J = 12.4 Hz, 2H), 1.35 (s, 3H), 1.14 (d, J = 7.4 Hz, |
| | 3H) |
| 174 | 1H NMR (400 MHz, DMSO) δ 10.94 (s, 1H), 8.97 (s, 1H), 8.38 (s, 1H), 8.01 (d, J = 8.1 Hz, 1H), |
| | 7.75-7.55 (m, 4H), 6.85 (d, J = 5.7 Hz, 1H), 5.96 (d, J = 15.8 Hz, 1H), 5.55 (d, J = 15.9 Hz, 1H), |
| | 4.09 (s, 2H), 3.84 (s, 2H), 3.38 (t, J = 7.3 Hz, 2H), 3.26 (d, J = 12.0 Hz, 2H), 2.99 (t, J = 11.5 Hz, |
| | 2H), 2.19 (t, J = 11.5 Hz, 2H), 1.63 (d, J = 12.4 Hz, 2H), 1.22 (s, 3H), 1.14 (t, J = 7.3 Hz, 3H). |
| 175 | ¹H NMR (400 MHz, DMSO-d₆) δ 12.50 (s, 1 H) 8.12-8.17 (m, 1 H) 8.09 (d, J = 8.5 Hz, 1 H) 7.80 |
| | (d, J = 1.5 Hz, 1 H) 7.58-7.68 (m, 2 H) 7.19 (s, 1 H) 7.10 (br d, J = 8.5 Hz, 1 H) 6.94 (br t, J = 6.4 |
| | Hz, 1 H) 6.51-6.56 (m, 1 H) 5.53-5.64 (m, 1 H) 5.32-5.46 (m, 1 H) 3.77 (t, J = 6.4 Hz, 2 H) |
| | 3.47-3.58 (m, 2 H) 3.33-3.36 (m, 2 H) 3.14 (br d, J = 11.0 Hz, 2 H) 2.77-2.91 (m, 2 H) 2.19- |
| | 2.29 (m, 1 H) 1.89-2.10 (m, 6 H) 1.52 (br d, J = 5.3 Hz, 2 H). |
| 176 | ¹H NMR (400 MHz, DMSO-d₆) δ 12.32 (br s, 1 H) 8.15 (d, J = 2.0 Hz, 1 H) 8.02 (br d, J = 8.5 Hz, |
| | 1 H) 7.79 (s, 1 H) 7.64 (s, 2 H) 7.17 (br d, J = 10.5 Hz, 2 H) 7.02-7.11 (m, 1 H) 6.54 (s, 1 H) 5.46- |
| | 5.62 (m, 2 H) 3.76 (br t, J = 6.4 Hz, 2 H) 3.36 (br d, J = 8.0 Hz, 2 H) 3.14-3.25 (m, 2 H) 2.65- |
| | 2.81 (m, 2 H) 2.41-2.47 (m, 2 H) 2.06-2.26 (m, 5 H) 1.83-2.00 (m, 2 H) 1.68 (br d, J = 2.8 Hz, |
| | 2 H). |
| 177 | ¹H NMR (499 MHz, DMSO-d₆) δ 10.64 (s, 1 H) 8.19 (d, J = 2.2 Hz, 1 H) 8.03 (d, J = 8.2 Hz, 1 H) |
| | 7.81 (d, J = 1.9 Hz, 1 H) 7.62-7.68 (m, 4 H) 7.07 (br s, 1 H) 6.50-6.61 (m, 1 H) 5.69 (dt, J = 15.3, |
| | 7.7 Hz, 1 H) 5.45-5.53 (m, 1 H) 3.73 (br s, 2 H) 3.33-3.46 (m, 5 H) 3.07-3.14 (m, 2 H) 3.01 |
| | (br t, J = 11.6 Hz, 2 H) 2.15-2.23 (m, 2 H) 1.97 (td, J = 12.9, 3.8 Hz, 2 H) 1.90 (d, J = 7.7 Hz, 2 H) |
| | 1.66-1.76 (m, 2 H) 1.13-1.21 (m, 2 H) 1.00 (s, 3 H). |
| 178 | ¹H NMR (499 MHz, DMSO-d₆) δ 12.63 (s, 1 H) 8.41 (d, J = 8.5 Hz, 1 H) 8.19 (d, J = 1.9 Hz, 1 H) |
| | 8.09 (d, J = 1.9 Hz, 1 H) 7.84 (dd, J = 8.4, 1.8 Hz, 1 H) 7.81 (d, J = 1.6 Hz, 1 H) 7.69 (d, J = 8.2 Hz, 1 |
| | H) 7.31 (d, J = 8.2 Hz, 1 H) 7.27 (br t, J = 5.7 Hz, 1 H) 6.53-6.60 (m, 1 H) 5.50-5.65 (m, 2 H) |
| | 3.37-3.45 (m, 7 H) 3.12-3.21 (m, 2 H) 2.99 (br d, J = 11.2 Hz, 2 H) 2.24-2.34 (m, 2 H) 2.08- |
| | 2.17 (m, 4 H) 1.66-1.77 (m, 2 H) 1.52 (br d, J = 13.1 Hz, 2 H) 1.17 (s, 3 H). |
| 208 | ¹H NMR (400 MHz, DMSO-d₆) δ 12.59 (s, 1H), 8.30 (d, J = 8.2 Hz, 1H), 7.97 (d, J = 1.8 Hz, |
| | 1H), 7.66 (d, J = 8.4 Hz, 1H), 7.62-7.61(m, 1H), 7.58 (d, J = 8.4 Hz, 1H), 7.46 (t, J = 6.4 Hz, |
| | 1H), 5.65-5.35 (m, 2H), 4.41 (s, 2H), 4.18-4.16 (m, 2H), 3.74-3.72 (m, 4H), 3.41-3.38 (m, 4H), |
| | 3.15-3.11 (m, 4H), 3.05-2.84 (m, 4H), 1.69 (d, J = 12.5 Hz, 4H), 1.35 (s, 3H), 1.15-1.12 (m, 3H). |
| 209 | ¹H NMR (400 MHz, DMSO-d₆) δ 12.62 (s, 1H), 8.34 (d, J = 8.2 Hz, 1H), 7.97 (d, J = 1.8 Hz, |
| | 1H), 7.66 (d, J = 8.4 Hz, 1H), 7.62-7.60 (m, 1H), 7.58 (d, J = 8.4 Hz, 1H), 7.46 (t, J = 6.4 Hz, |
| | 1H), 5.90-5.53 (m, 2H), 4.41 (s, 2H), 4.18 (d, J = 4.6 Hz, 2H), 3.74 (dd, J = 5.9, 3.6 Hz, 4H), |
| | 3.41-3.39 (m, 4H), 3.14-3.10 (m, 4H), 3.05-2.84 (m, 4H), 1.69 (d, J = 12.5 Hz, 4H), 1.35 (s, 3H), |
| | 1.15 (t, J = 7.4 Hz, 3H). |
| 210 | ¹H NMR (400 MHz, DMSO-d₆) δ 10.87 (s, 1H), 8.12 (s, 1H), 8.01 (d, J = 8.1 Hz, 1H), 7.76-7.56 |
| | (m, 5H), 7.41 (t, J = 5.5 Hz, 1H), 5.98 (d, J = 15.7 Hz, 1H), 5.53 (d, J = 15.8 Hz, 1H), 4.09 (s, |
| | 2H), 3.92 (s, 2H), 3.59 (t, J = 4.6 Hz, 4H), 3.46-3.32 (m, 4H), 3.25 (d, J = 11.8 Hz, 2H), 3.04-2.93 |
| | (m, 2H), 2.40 (s, 2H), 2.20 (t, J = 11.0 Hz, 3H), 1.63 (d, J = 12.3 Hz, 2H), 1.24 (s, 1H), 1.22 (s, |
| | 3H), 1.14 (t, J = 7.3 Hz, 3H). |
| 211 | ¹H NMR (400 MHz, DMSO-d₆) δ 12.65 (s, 1H), 8.35 (d, J = 8.2 Hz, 1H), 8.08 (s, 1H), 7.97 (d, J = |
| | 1.8 Hz, 1H), 7.81 (dd, J = 8.3, 1.7 Hz, 1H), 7.73-7.66 (m, 2H), 7.58 (d, J = 8.3 Hz, 1H), 7.31 (t, |
| | J = 6.3 Hz, 1H), 5.55-5.48 (m, 1H), 5.40-5.32 (m, 1H), 4.40 (s, 2H), 4.21-4.16 (m, 2H), 3.59 (t, J = |
| | 4.7 Hz, 4H), 3.46-3.33 (m, 4H), 3.18 (d, J = 11.4 Hz, 2H), 3.08 (t, J = 11.9 Hz, 2H), 2.40 (s, |
| | 4H), 1.69 (d, J = 12.3 Hz, 2H), 1.35 (s, 3H), 1.23 (d, J = 6.4 Hz, 2H), 1.15 (t, J = 7.3 Hz, 3H). |
| 213 | ¹H NMR (400 MHz, DMSO) δ 12.25 (s, 1H), 8.33 (d, J = 8.2 Hz, 1H), 8.01 (d, J = 2.4 Hz, 1H), |
| | 7.93 (d, J = 1.8 Hz, 1H), 7.78 (dd, J = 8.2, 1.7 Hz, 1H), 7.55-7.46 (m, 2H), 6.99 (d, J = 8.2 Hz, |
| | 1H), 6.32 (t, J = 2.1 Hz, 1H), 5.92 (t, J = 6.3 Hz, 1H), 5.53-5.35 (m, 2H), 5.30-5.21 (m, 1H), |
| | 4.34-4.24 (m, 2H), 4.20 (d, J = 4.7 Hz, 2H), 4.00 (t, J = 7.0 Hz, 2H), 3.40 (q, J = 7.4 Hz, 2H), |
| | 3.15 (d, J = 10.8 Hz, 2H), 3.12-3.00 (m, 2H), 2.68 (s, 1H), 2.32 (d, J = 9.7 Hz, 3H), 1.64 (d, J = |
| | 12.3 Hz, 2H), 1.34 (s, 3H), 1.14 (t, J = 7.3 Hz, 3H). |
| 214 | ¹H NMR (400 MHz, DMSO) δ 10.69 (s, 1H), 8.03 (d, J = 7.9 Hz, 1H), 7.74-7.56 (m, 1H), 7.56- |
| | 7.42 (m, 1H), 6.96 (d, J = 8.2 Hz, 1H), 6.32 (s, 1H), 6.10-5.90 (m, 1H), 5.49 (d, J = 15.7 Hz, 1H), |
| | 5.27 (t, J = 6.6 Hz, 1H), 4.33 (t, J = 7.4 Hz, 1H), 4.15-3.99 (m, 3H), 3.84 (s, 1H), 3.33-3.30 (m, 4 |
| | H), 3.29-3.12 (m, 1H), 2.96 (t, J = 11.7 Hz, 2H), 2.68 (s, 4H), 2.33 (s, 1H), 2.22 (t, J = 11.8 Hz, |
| | 1H), 2.08 (s, 5H), 1.61 (d, J = 12.2 Hz, 1H), 1.39-1.08 (m, 4H). |
| 215 | ¹H NMR (400 MHz, DMSO-d₆) δ 10.83 (s, 1H), 8.01 (d, J = 8.1 Hz, 1H), 7.70-7.55 (m, 7H), 7.37 |
| | (s, 1H), 5.96 (s, 1H), 5.54 (d, J = 15.6 Hz, 1H), 5.38 (s, 1H), 4.10 (s, 3H), 3.93 (s, 3H), 3.59 (s, |
| | 2H), 3.23 (s, 2H), 3.00 (d, J = 11.8 Hz, 2H), 2.20 (s, 2H), 1.62 (d, J = 12.1 Hz, 2H), 1.23 (d, J = |
| | 10.9 Hz, 4H), 1.14 (t, J = 7.4 Hz, 4H). |
| 216 | ¹H NMR (400 MHz, DMSO-d₆) δ 12.60 (s, 1H), 8.34 (d, J = 8.2 Hz, 1H), 7.96 (d, J = 1.7 Hz, |
| | 1H), 7.80 (dd, J = 8.2, 1.7 Hz, 1H), 7.64 (d, J = 8.4 Hz, 1H), 7.60-7.51 (m, 3H), 7.35 (s, 1H), 5.51 |
| | (s, 1H), 5.46-5.36 (m, 1H), 5.28 (d, J = 8.6 Hz, 1H), 4.41 (s, 2H), 4.18 (d, J = 4.8 Hz, 2H), 4.02 |
| | (d, J = 7.4 Hz, 1H), 3.74 (t, J = 7.6 Hz, 2H), 3.47-3.37 (m, 4H), 3.16 (s, 2H), 3.08 (t, J = 12.0 Hz, |
| | 2H), 1.69 (d, J = 12.4 Hz, 2H), 1.35 (s, 3H), 1.24 (s, 2H), 1.15 (t, J = 7.3 Hz, 3H), 0.85 (d, J = 7.1 |
| | Hz, 1H). |
| 222 | ¹H NMR (400 MHz, DMSO-d6) δ 10.86 (s, 1H), 8.03 (d, J = 8.1 Hz, 1H), 7.83 (s, 1H), 7.72-7.62 |
| | (m, 2H), 7.44-7.34 (m, 2H), 7.08 (d, J = 8.4 Hz, 1H), 6.90 (d, J = 5.4 Hz, 1H), 5.99 (d, J = 15.6 |
| | Hz, 1H), 5.54 (d, J = 16.2 Hz, 1H), 4.10 (s, 2H), 3.85 (s, 2H), 3.70-3.65 (m, 1H), 3.37-3.35 (m, |
| | 2H), 3.21-3.19 (m, 2H), 2.97 (t, J = 11.8 Hz, 2H), 2.21 (t, J = 10.8 Hz, 2H), 1.63 (d, J = 12.3 Hz, |
| | 2H), 1.23 (s, 3H), 1.13 (t, J = 7.3 Hz, 3H), 1.08-1.00 (m, 2H), 0.95-0.92 (m, 2H). |
| 224 | ¹H NMR (400 MHz, DMSO-d6) δ 12.43 (s, 1H), 8.03 (d, J = 8.1 Hz, 1H), 7.83 (s, 1H), 7.73-7.61 |
| | (m, 2H), 7.48-7.32 (m, 2H), 7.08 (d, J = 8.4 Hz, 1H), 6.90 (d, J = 5.4 Hz, 1H), 5.53-5.38(m, 2H), |
| | 4.10 (s, 2H), 3.85 (s, 2H), 3.70-3.65 (m, 1H), 3.37-3.35 (m, 2H), 3.21 (d, J = 11.7 Hz, 2H), 2.97 |
| | (t, J = 11.8 Hz, 2H), 2.33-2.12 (m, 2H), 1.63 (d, J = 12.3 Hz, 2H), 1.23 (s, 3H), 1.13 (t, J = 7.3 |
| | Hz, 3H), 1.04 (q, J = 4.0 Hz, 2H), 0.95 (dd, J = 7.4, 4.8 Hz, 2H). |
| 226 | ¹H NMR (400 MHz, DMSO-d₆) δ 10.86 (s, 1H), 8.01 (d, J = 8.0 Hz, 1H), 7.92 (s, 1H), 7.70-7.60 |
| | (m, 3H), 7.64-7.54 (m, 1H), 7.37 (s, 1H), 5.98 (d, J = 16.1 Hz, 1H), 5.53 (d, J = 15.7 Hz, 1H), |
| | 4.88-4.80 (m, 1H), 4.09 (s, 3H), 3.90 (s, 3H), 3.78 (s, 3H), 3.55 (s, 2H), 3.37 (q, J = 7.3 Hz, 2H), |
| | 3.25 (d, J = 12.4 Hz, 3H), 3.01-2.95 (m, 3H), 2.19 (s, 2H), 1.62 (d, J = 12.7 Hz, 2H), 1.15-1.11 |
| | (m, 3H). |
| 225 | ¹H NMR (400 MHz, DMSO-d₆) δ 12.64 (s, 1H), 8.34 (d, J = 8.2 Hz, 1H), 7.97 (d, J = 1.8 Hz, |
| | 1H), 7.89 (s, 1H), 7.80 (dd, J = 8.2, 1.8 Hz, 1H), 7.64 (d, J = 8.4 Hz, 1H), 7.57 (d, J = 8.6 Hz, |
| | 2H), 5.50 (s, 1H), 5.40 (s, 1H), 4.85-4.82 (m, 1H), 4.39 (s, 2H), 4.18 (s, 2H), 3.78 (s, 2H), 3.56 (s, |
| | 2H), 3.41 (q, J = 7.3 Hz, 2H), 3.32 (s, 3H), 3.15-3.05 (m, 4H), 1.69 (d, J = 12.3 Hz, 2H), 1.35 (s, |
| | 3H), 1.24 (s, 1H), 1.16-1.12 (m, 3H). |
| 227 | ¹H NMR (400 MHz, DMSO-d6) δ 10.88 (s, 1H), 8.21 (d, J = 0.8 Hz, 1H), 8.01 (d, J = 8.1 Hz, |
| | 1H), 7.79 (d, J = 0.8 Hz, 1H), 7.72 (d, J = 8.3 Hz, 1H), 7.66 (dd, J = 11.7, 1.7 Hz, 2H), 7.64-7.56 |
| | (m, 1H), 7.27 (d, J = 5.5 Hz, 1H), 5.98 (d, J = 16.0 Hz, 1H), 5.54 (d, J = 16.1 Hz, 1H), 4.17-4.04 |
| | (m, 2H), 3.98-3.84 (m, 2H), 3.65 (t, J = 6.8 Hz, 2H), 3.45 (t, J = 6.9 Hz, 2H), 3.42-3.34 (m, 2H), |
| | 3.29-3.22 (m, 2H), 2.99 (t, J = 11.5 Hz, 2H), 2.48-2.38 (m, 1H), 2.32 (d, J = 9.4 Hz, 1H), 2.25- |
| | 2.13 (m, 2H), 1.63 (d, J = 12.1 Hz, 2H), 1.22 (s, 3H), 1.14 (t, J = 7.4 Hz, 3H). |
| 229 | ¹H NMR (400 MHz, DMSO-d₆) δ 10.95 (s, 1H), 8.85 (s, 1H), 8.22 (s, 1H), 8.00 (d, J = 8.1 Hz, |
| | 1H), 7.75 (d, J = 8.3 Hz, 1H), 7.71 (d, J = 1.8 Hz, 1H), 7.66 (dd, J = 8.1, 1.7 Hz, 1H), 7.62 (d, J = |
| | 8.3 Hz, 1H), 7.03 (t, J = 5.4 Hz, 1H), 5.99 (s, 1H), 5.55 (d, J = 16.0 Hz, 1H), 4.95 (s, 1H), 4.10 (s, |
| | 2H), 3.88 (s, 2H), 3.72 (t, J = 6.3 Hz, 2H), 3.53 (t, J = 6.3 Hz, 2H), 2.99 (t, J = 11.6 Hz, 3H), 2.88 |
| | (q, J = 7.3 Hz, 3H), 2.21 (t, J = 11.3 Hz, 2H), 1.64 (d, J = 12.4 Hz, 2H), 1.22 (s, 3H), 1.09 (t, J = |
| | 7.3 Hz, 3H) |
| 230 | ¹H NMR (400 MHz, DMSO-d6) δ 12.64 (s, 1H), 8.35 (d, J = 8.2 Hz, 1H), 7.96 (dd, J = 10.4, 1.3 |
| | Hz, 2H), 7.81 (dd, J = 8.2, 1.7 Hz, 1H), 7.67 (d, J = 8.4 Hz, 1H), 7.62 (d, J = 0.8 Hz, 1H), 7.58 (d, |
| | J = 8.3 Hz, 1H), 7.34 (t, J = 6.3 Hz, 1H), 5.93 (s, 1H), 5.52 (d, J = 11.7 Hz, 1H), 5.46-5.32 (m, |
| | 1H), 4.39 (d, J = 6.6 Hz, 2H), 4.18 (d, J = 4.7 Hz, 2H), 3.41-3.39 (m, 2H), 3.18 (d, J = 10.9 Hz, |
| | 2H), 3.08 (t, J = 12.0 Hz, 2H), 1.69 (d, J = 12.3 Hz, 2H), 1.35 (s, 3H), 1.15 (t, J = 7.3 Hz, 3H), |
| | 1.09-0.97 (m, 3H), 0.91-0.78 (m, 3H). |
| 260 | ¹H NMR (400 MHz, DMSO-d₆) δ 12.22 (s, 1H), 8.31 (d, J = 8.2 Hz, 1H), 7.92 (d, J = 1.8 Hz, |
| | 1H), 7.77 (dd, J = 8.2, 1.8 Hz, 1H), 7.60 (d, J = 0.9 Hz, 1H), 7.42 (d, J = 8.2 Hz, 1H), 7.26 (d, J = |
| | 0.9 Hz, 1H), 6.99 (d, J = 8.2 Hz, 1H), 6.52 (s, 1H), 6.24 (s, 1H), 5.59-5.38 (m, 2H), 4.36 (s, 2H), |
| | 4.19 (d, J = 4.7 Hz, 2H), 3.67-6.52 (m, 1H), 3.39 (q, J = 7.3 Hz, 3H), 3.14 (s, 1H), 3.06 (t, J = |
| | 12.0 Hz, 2H), 2.62-2.52 (m, 2H), 1.67 (d, J = 12.3 Hz, 2H), 1.35 (s, 3H), 1.14 (t, J = 7.4 Hz, 3H), |
| | 1.07-1.00 (m, 2H), 0.98-0.89 (m, 2H). |
| 261 | ¹H NMR (400 MHz, DMSO-d₆) δ 12.22 (s, 1H), 8.31 (d, J = 8.2 Hz, 1H), 7.92 (d, J = 1.8 Hz, |
| | 1H), 7.77 (dd, J = 8.2, 1.8 Hz, 1H), 7.60 (d, J = 0.9 Hz, 1H), 7.42 (d, J = 8.2 Hz, 1H), 7.26 (d, J = |
| | 0.9 Hz, 1H), 6.99 (d, J = 8.2 Hz, 1H), 6.52 (s, 1H), 6.24 (s, 1H), 5.64-5.37 (m, 2H), 4.36 (s, 2H), |
| | 4.19 (d, J = 4.7 Hz, 2H), 3.75-3.60 (m, 1H), 3.46-3.35 (m, 6H), 3.14 (s, 2H), 3.06 (t, J = 12.0 Hz, |
| | 2H), 2.62-2.54 (m, 1H), 1.67 (d, J = 12.3 Hz, 2H), 1.16-1.10 (m, 3H), 1.07-1.00 (m, 2H), 0.98- |
| | 0.85 (m, 2H). |
| 271 | ¹H NMR (400 MHz, DMSO-d6) δ 10.86 (s, 1H), 8.06-7.92 (m, 2H), 7.75-7.66 (m, 2H), 7.66-7.60 |
| | (m, 2H), 7.58 (s, 1H), 7.42 (s, 1H), 6.03-5.91 (m, 2H), 5.53 (d, J = 15.9 Hz, 1H), 4.10 (s, 2H), |
| | 3.92 (s, 2H), 3.38 (t, J = 7.4 Hz, 2H), 2.98 (t, J = 11.6 Hz, 2H), 2.20 (t, J = 11.5 Hz, 2H), 1.63 (d, |
| | J = 12.4 Hz, 2H), 1.22 (s, 3H), 1.14 (t, J = 7.3 Hz, 3H), 1.03 (q, J = 4.7 Hz, 3H), 0.86 (t, J = 3.6 |
| | Hz, 3H). |
| 274 | ¹H NMR (400 MHz, DMSO-d₆) δ 12.69 (s, 1H), 8.35 (d, J = 8.2 Hz, 1H), 8.17 (d, J = 0.8 Hz, |
| | 1H), 7.98 (d, J = 1.8 Hz, 1H), 7.81 (dd, J = 8.1, 1.3 Hz, 2H), 7.69 (d, J = 8.3 Hz, 1H), 7.60 (d, J = |
| | 8.3 Hz, 1H), 7.19 (d, J = 6.1 Hz, 1H), 5.57-5.46 (m, 1H), 5.46-5.33 (m, 1H), 4.40 (s, 2H), 4.19 (d, |
| | J = 4.6 Hz, 2H), 3.65 (t, J = 6.8 Hz, 2H), 3.49-3.38 (m, 4H), 3.22-3.04 (m, 4H), 2.48-2.39 (m, |
| | 2H), 2.34-2.30 (m, 1H), 1.69 (d, J = 12.5 Hz, 2H), 1.35 (s, 3H), 1.24 (s, 1H), 1.15 (t, J = 7.3 Hz, |
| | 3H). |
| 282 | ¹H NMR (400 MHz, DMSO-d₆) δ 10.91 (s, 2H), 7.97-7.81 (m, 2H), 7.67-7.55 (m, 2H), 7.23 (s, |
| | 1H), 7.11-7.04 (m, 1H), 7.01 (d, J = 8.7 Hz, 1H), 5.93 (d, J = 4.4 Hz, 1H), 5.56 (m, 2H), 5.33 (m, |
| | 1H), 3.67 (s, 2H), 3.18 (m, 3H), 2.73-2.62 (m, 2H), 2.37-2.27 (m, 1H), 2.20 (d, J = 25.7 Hz, 2H), |
| | 2.10-1.93 (m, 3H), 1.81 (m, 2H), 1.66 (s, 1H), 1.49 (dd, J = 29.2, 9.5 Hz, 3H), 1.07-0.97 (m, 2H), |
| | 0.85 (m, 3H). |
| 299 | ¹H (400 MHz, DMSO-d₆) δ 12.56 (s, 1H), 8.09 (d, J = 8.6 Hz, 1H), 7.79 (s, 1H), 7.67 (d, J = 2.0 |
| | Hz, 1H), 7.65-7.59 (m, 2H), 7.55 (dd, J = 8.5, 2.0 Hz, 1H), 7.45 (t, J = 6.5 Hz, 1H), 7.33 (d, J = |
| | 8.2 Hz, 1H), 5.50 (t, J = 5.6 Hz, 2H), 3.80-3.69 (m, 4H), 3.46 (dd, J = 10.7, 5.9 Hz, 2H), 3.09 (d, J = |
| | 11.2 Hz, 2H), 3.01-2.83 (m, 6H), 2.26 (dd, J = 11.0, 5.9 Hz, 2H), 2.20-2.12 (m, 2H), 1.90-1.87 |
| | (m, 1H), 1.79 (d, J = 12.9 Hz, 2H), 1.74-1.63 (m, 2H). |
| 301 | ¹H (400 MHz, DMSO-d₆) δ 10.83 (s, 1H), 7.98 (d, J = 8.1 Hz, 1H), 7.77 (d, J = 8.3 Hz, 1H), 7.66 |
| | (d, J = 1.7 Hz, 1H), 7.62 (dd, J = 8.0, 1.6 Hz, 1H), 7.45 (d, J = 8.2 Hz, 1H), 6.82 (t, J = 5.3 Hz, |
| | 1H), 5.94 (dt, J = 15.6, 4.1 Hz, 1H), 5.54-5.43 (m, 1H), 4.08 (dd, J = 5.0, 2.3 Hz, 2H), 3.85 (s, |
| | 2H), 3.36 (q, J = 7.3 Hz, 2H), 3.22 (dd, J = 10.0, 6.2 Hz, 2H), 2.96 (t, J = 11.4 Hz, 2H), 2.21- |
| | 2.06 (m, 2H), 1.61 (d, J = 12.3 Hz, 2H), 1.20 (s, 3H), 1.13 (t, J = 7.3 Hz, 3H). |
| 302 | ¹H (400 MHz, DMSO-d₆) δ 12.61 (s, 1H), 8.32 (d, J = 8.2 Hz, 1H), 7.96 (d, J = 1.8 Hz, 1H), 7.79 |
| | (dd, J = 8.2, 1.7 Hz, 1H), 7.75 (d, J = 8.3 Hz, 1H), 7.43 (d, J = 8.3 Hz, 1H), 6.64 (t, J = 6.2 Hz, |
| | 1H), 5.54-5.46 (m, 1H), 5.43 (td, J = 12.0, 11.2, 6.5 Hz, 1H), 4.32 (t, J = 5.9 Hz, 2H), 4.16 (d, J = |
| | 4.3 Hz, 2H), 3.40 (q, J = 7.3 Hz, 2H), 3.14 (d, J = 13.0 Hz, 2H), 3.10-3.00 (m, 2H), 2.45 (dd, J = |
| | 12.8, 4.8 Hz, 2H), 1.66 (d, J = 12.4 Hz, 2H), 1.33 (s, 3H), 1.14 (t, J = 7.3 Hz, 3H). |
| 304 | ¹H (400 MHz, DMSO-d₆,): δ 10.77 (s, 1H), 10.19 (s, 1H), 7.85 (d, J = 8.5 Hz, 1H), 7.69 (d, J = |
| | 8.3 Hz, 1H), 7.49 (d, J = 8.3 Hz, 1H), 7.07 (d, J = 2.1 Hz, 1H), 7.00 (dd, J = 8.5, 2.0 Hz, 1H), |
| | 6.36 (t, J = 6.6 Hz, 1H), 5.52 (tdd, J = 22.2, 15.2, 7.1 Hz, 2H), 3.61 (q, J = 6.2 Hz, 2H), 3.18 (td, |
| | J = 13.2, 8.2 Hz, 4H), 2.65 (t, J = 11.4 Hz, 2H), 2.24-2.16 (m, 2H), 2.03 (dd, J = 7.7, 3.6 Hz, |
| | 2H), 1.82-1.60 (m, 3H), 1.50 (d, J = 12.3 Hz, 2H), 1.2 (t, J = 7.3 Hz, 3H). |
| 310 | ¹H NMR: (400 MHz, DMSO-d₆): δ 11.08 (s, 1H), 10.20 (s, 1H), 7.85 (d, J = 8.5 Hz, 1H), 7.73 (d, |
| | J = 8.3 Hz, 1H), 7.47 (d, J = 8.3 Hz, 1H), 7.14 (d, J = 2.1 Hz, 1H), 7.03 (dd, J = 8.5, 2.0 Hz, 1H), |
| | 6.77 (t, J = 5.3 Hz, 1H), 5.95 (d, J = 15.5 Hz, 1H), 5.50 (dd, J = 12.9, 8.1 Hz, 1H), 4.15-4.07 |
| | (m, 2H), 3.85 (s, 2H), 3.29-3.07 (m, 4H), 2.78 (t, J = 12.0 Hz, 2H), 2.23 (dt, J = 13.6, 6.9 Hz, |
| | 2H), 1.63 (d, J = 12.1 Hz, 2H), 1.30-1.10 (m, 6H). |
| 311 | ¹H NMR: (400 MHz, DMSO-d₆): δ 11.08 (s, 1H), 10.20 (s, 1H), 7.85 (d, J = 8.5 Hz, 1H), 7.73 (d, |
| | J = 8.3 Hz, 1H), 7.47 (d, J = 8.3 Hz, 1H), 7.14 (d, J = 2.1 Hz, 1H), 7.03 (dd, J = 8.5, 2.0 Hz, 1H), |
| | 6.77 (t, J = 5.3 Hz, 1H), 5.95 (d, J = 15.5 Hz, 1H), 5.50 (dd, J = 12.9, 8.1 Hz, 1H), 4.15-4.07 |
| | (m, 2H), 3.85 (s, 2H), 3.29-3.07 (m, 4H), 2.78 (t, J = 12.0 Hz, 2H), 2.23 (dt, J = 13.6, 6.9 Hz, |
| | 2H), 1.63 (d, J = 12.1 Hz, 2H), 1.30-1.10 (m, 6H). |
| 351 | ¹H NMR (400 MHz, DMSO-d6) δ 10.73 (s, 1H), 8.81 (s, 1H), 8.23 (s, 1H), 8.06 (d, J = 8.0 Hz, |
| | 1H), 7.66 (dd, J = 11.4, 6.2 Hz, 3H), 7.59 (d, J = 1.8 Hz, 1H), 6.68 (t, J = 6.6 Hz, 1H), 5.54-5.16 |
| | (m, 2H), 3.66 (s, 2H), 3.42-3.35 (m, 3H), 3.29 (m, 2H), 2.93-2.85 (m, 2H), 2.85-2.77 (m, 1H), |
| | 2.16 (s, 2H), 2.06 (d, J = 4.1 Hz, 2H), 1.79 (q, J = 12.2 Hz, 2H), 1.67 (d, J = 11.7 Hz, 1H), 1.53 |
| | (d, J = 12.2 Hz, 2H), 1.11-1.00 (m, 6H). |
| 352 | ¹H NMR (400 MHz, DMSO-d₆) δ 12.58 (s, 1H), 10.26 (s, 1H), 8.08 (d, J = 8.7 Hz, 1H), 7.92 (s, |
| | 1H), 7.65-7.57 (m, 2H), 7.31-7.18 (m, 2H), 7.13 (dd, J = 8.7, 2.1 Hz, 1H), 5.92 (s, 1H), 5.51 (d, J = |
| | 11.3 Hz, 1H), 5.43-5.26 (m, 2H), 4.38 (d, J = 6.0 Hz, 2H), 4.22-4.10 (m, 2H), 3.22 (m, 2H), |
| | 3.12 (d, J = 11.5 Hz, 2H), 2.87 (m, 2H), 2.00 (m, 3H), 1.69 (d, J = 12.3 Hz, 2H), 1.46 (m, J = 7.2 |
| | Hz, 2H), 1.09-0.97 (m, 2H), 0.86 (m, J = 5.5, 3.2 Hz, 5H). |
| 353 | ¹H NMR (400 MHz, DMSO-d₆) δ 11.12 (s, 1H), 10.20 (s, 1H), 7.96 (s, 1H), 7.86 (d, J = 8.6 Hz, |
| | 1H), 7.70-7.56 (m, 3H), 7.37 (m, J = 5.3 Hz, 1H), 7.14 (s, 1H), 7.03 (dd, J = 8.5, 2.0 Hz, 1H), |
| | 5.98 (d, J = 15.8 Hz, 1H), 5.92 (s, 1H), 5.53 (d, J = 16.0 Hz, 1H), 4.12 (s, 2H), 3.90 (s, 2H), 3.21 |
| | (m, 2H), 3.14 (d, J = 11.8 Hz, 2H), 2.80 (m, 2H), 2.35-2.21 (m, 4H), 2.01 (m, 2H), 1.64 (d, J = |
| | 12.1 Hz, 2H), 1.06-0.99 (m, 2H), 0.86 (m, 4H). |
| 354 | ¹H NMR (400 MHz, DMSO) δ 12.73 (s, 1H), 8.79 (s, 1H), 8.35 (d, J = 8.2 Hz, 1H), 8.23 (s, 1H), |
| | 7.98 (d, J = 1.8 Hz, 1H), 7.81 (dd, J = 8.2, 1.8 Hz, 1H), 7.72 (d, J = 8.3 Hz, 1H), 7.61 (d, J = 8.3 |
| | Hz, 1H), 6.88 (t, J = 6.3 Hz, 1H), 5.65-5.46 (m, 1H), 5.42-5.27 (m, 1H), 4.41-4.28 (m, 2H), 4.18 |
| | (d, J = 4.7 Hz, 2H), 3.41-3.32 (m, 4H), 3.18 (d, J = 11.3 Hz, 2H), 3.09 (t, J = 12.0 Hz, 2H), 2.94- |
| | 2.83 (m, 2H), 1.76-1.62 (m, 2H), 1.35 (s, 3H), 1.20-1.02 (m, 6H). |
| 356 | ¹H NMR (400 MHz, DMSO-d₆) δ 10.68 (s, 1H), 8.03 (d, J = 8.1 Hz, 1H), 7.73-7.58 (m, 3H), 7.44 |
| | (d, J = 8.2 Hz, 1H), 7.29 (d, J = 0.9 Hz, 1H), 6.99 (d, J = 8.2 Hz, 1H), 6.54 (s, 1H), 6.42 (s, 1H), |
| | 6.03 (d, J = 16.0 Hz, 1H), 5.54 (d, J = 15.3 Hz, 1H), 4.11 (s, 2H), 3.88 (s, 2H), 3.73-3.62 (m, 1H), |
| | 3.27-3.14 (m, 4H), 3.01-2.95 (m, 2H), 2.93 (s, 1H), 2.75-2.64 (m, 1H), 2.37-2.31 (m, 1H), 2.27- |
| | 2.18 (m, 2H), 1.04-1.02 (m, 2H), 1.12-0.89 (m, 2H), 0.94-0.82 (m, 1H), 1.23 (d, J = 7.4 Hz, 2H), |
| | 1.62 (d, J = 12.1 Hz, 2H). |
| 357 | ¹H NMR (400 MHz, DMSO) δ 12.22 (s, 1H), 8.31 (d, J = 8.2 Hz, 1H), 7.92 (d, J = 1.8 Hz, 1H), |
| | 7.77 (dd, J = 8.2, 1.7 Hz, 1H), 7.60 (d, J = 0.9 Hz, 1H), 7.42 (d, J = 8.3 Hz, 1H), 7.26 (d, J = 0.9 |
| | Hz, 1H), 6.99 (d, J = 8.2 Hz, 2H), 6.51 (s, 2H), 6.24 (t, J = 6.1 Hz, 1H), 5.58-5.35 (m, 2H), 4.36 |
| | (s, 2H), 4.19 (d, J = 4.7 Hz, 2H), 3.67 (m, 2H), 3.40 (t, J = 7.3 Hz, 2H), 3.21-2.98 (m, 2H), 2.55 |
| | (s, 1H), 2.32 (d, J = 10.1 Hz, 2H), 1.67 (d, J = 12.4 Hz, 2H), 1.35 (s, 3H), 1.03-1.01 (m, 2H), |
| | 0.93-0.90 (m, 2H), 0.88-0.80 (m, 1H). |
| 358 | ¹H NMR (400 MHz, DMSO-d₆) δ 12.70 (s, 1H), 8.35 (d, J = 8.2 Hz, 1H), 8.11 (d, J = 2.5 Hz, |
| | 1H), 7.97 (d, J = 1.8 Hz, 1H), 7.81 (dd, J = 8.2, 1.8 Hz, 1H), 7.72 (d, J = 8.3 Hz, 1H), 7.59 (d, J = |
| | 8.3 Hz, 1H), 7.45 (d, J = 6.3 Hz, 1H), 6.50 (d, J = 2.4 Hz, 1H), 5.60 (s, 2H), 5.45-5.36 (m, 1H), |
| | 4.45 (s, 2H), 4.18 (s, 1H), 3.41-3.40 (m, 4H), 3.17 (s, 2H), 3.09-3.08 (m, 4H), 2.47 (d, J = 9.0 Hz, |
| | 1H), 2.33-2.22 (m, 2H), 1.82 (s, 1H), 1.76-1.61 (m, 3H), 1.35 (s, 3H), 1.15 (d, J = 7.3 Hz, 3H). |
| 359 | ¹ H NMR (400 MHz, DMSO-d₆) δ 12.69 (s, 1H), 8.34 (d, J = 8.2 Hz, 1H), 8.08 (d, J = 2.6 Hz, |
| | 1H), 7.97 (d, J = 1.8 Hz, 1H), 7.81 (dd, J = 8.3, 1.7 Hz, 1H), 7.65 (d, J = 8.3 Hz, 1H), 7.57 (d, J = |
| | 8.3 Hz, 1H), 7.09 (t, J = 6.1 Hz, 1H), 6.31 (d, J = 2.6 Hz, 1H), 5.52 (d, J = 11.8 Hz, 1H), 5.47- |
| | 5.38 (m, 1H), 4.40 (d, J = 6.4 Hz, 2H), 4.18 (d, J = 4.5 Hz, 2H), 3.84 (t, J = 6.7 Hz, 2H), 3.51 (t, J = |
| | 7.4 Hz, 2H), 3.42 (t, J = 7.3 Hz, 2H), 3.18 (d, J = 11.8 Hz, 2H), 3.08 (t, J = 12.0 Hz, 2H), 2.42 |
| | (m, 2H), 1.71 (d, J = 12.4 Hz, 2H), 1.35 (s, 3H), 1.24 (s, 2H), 1.14 (t, J = 7.3 Hz, 3H). |
| 360 | ¹H NMR (400 MHz, DMSO-d₆) δ 10.92 (s, 1H), 8.16-7.94 (m, 2H), 7.83-7.41 (m, 4H), 7.16 (d, J = |
| | 5.8 Hz, 2H), 6.31 (d, J = 2.6 Hz, 1H), 6.00 (d, J = 16.0 Hz, 1H), 5.54 (d, J = 15.7 Hz, 2H), 4.09 |
| | (s, 2H), 3.90 (s, 2H), 3.84 (t, J = 6.7 Hz, 2H), 3.50 (t, J = 7.4 Hz, 2H), 3.25 (d, J = 11.8 Hz, 2H), |
| | 2.99 (t, J = 11.5 Hz, 2H), 2.41 (m, 2H), 2.20 (t, J = 11.7 Hz, 2H), 1.64 (d, J = 12.3 Hz, 2H), 1.22 |
| | (s, 3H), 1.14 (t, J = 7.3 Hz, 3H) |
| 361 | ¹H NMR (400 MHz, DMSO-d₆) δ 10.66 (s, 1H), 8.13-8.01 (m, 2H), 7.76-7.45 (m, 4H), 6.91 (t, J = |
| | 6.7 Hz, 1H), 6.31 (d, J = 2.5 Hz, 1H), 5.54 (m, 2H), 3.83 (t, J = 6.7 Hz, 2H), 3.67 (d, J = 8.9 |
| | Hz, 2H), 3.51 (t, J = 7.4 Hz, 2H), 3.28 (d, J = 11.1 Hz, 2H), 2.81 (t, J = 11.5 Hz, 2H), 2.43 (m, |
| | 2H), 2.19 (s, 2H), 2.08 (s, 4H), 1.77 (dd, J = 13.5, 9.9 Hz, 2H), 1.66 (d, J = 12.4 Hz, 1H), 1.52 (d, |
| | J = 12.2 Hz, 2H), 1.13 (t, J = 7.3 Hz, 3H) |
| 362 | ¹H NMR (400 MHz, DMSO-d₆) δ 12.60 (s, 1H), 8.34 (d, J = 8.2 Hz, 1H), 7.97 (dd, J = 7.7, 2.2 |
| | Hz, 2H), 7.80 (dd, J = 8.2, 1.7 Hz, 1H), 7.57 (q, J = 8.3 Hz, 2H), 7.39 (t, J = 6.2 Hz, 1H), 6.10 (d, |
| | J = 2.6 Hz, 1H), 5.52 (d, J = 11.4 Hz, 1H), 5.42 (q, J = 5.8 Hz, 1H), 4.41 (s, 2H), 4.23-4.13 (m, |
| | 2H), 3.73 (t, J = 4.8 Hz, 4H), 3.41 (q, J = 7.3 Hz, 2H), 3.23-3.13 (m, 6H), 3.08 (t, J = 12.1 Hz, |
| | 2H), 2.31 (s, 1H), 2.08 (s, 1H), 1.70 (d, J = 12.4 Hz, 2H), 1.35 (s, 3H), 1.14 (t, J = 7.3 Hz, 3H) |
| 363 | ¹H NMR (400 MHz, DMSO-d₆) δ 10.88 (s, 1H), 8.02 (d, J = 8.4 Hz, 2H), 7.76-7.52 (m, 4H), 7.44 |
| | (s, 1H), 6.10 (s, 1H), 6.01 (d, J = 15.8 Hz, 1H), 5.54 (d, J = 15.6 Hz, 1H), 4.10 (s, 2H), 3.93 (s, |
| | 2H), 3.72 (d, J = 5.0 Hz, 2H), 3.37 (d, J = 7.8 Hz, 3H), 3.23 (s, 2H), 3.17 (t, J = 4.7 Hz, 3H), 2.98 |
| | (t, J = 11.8 Hz, 2H), 2.31 (s, 2H), 2.20 (s, 2H), 1.63 (d, J = 12.3 Hz, 2H), 1.22 (s, 3H), 1.13 (t, J = |
| | 7.3 Hz, 3H) |
| 364 | ¹H NMR (400 MHz, DMSO) δ 10.64 (s, 1H), 8.06 (d, J = 8.0 Hz, 1H), 7.98 (d, J = 2.6 Hz, 1H), |
| | 7.67-7.51 (m, 3H), 7.20 (t, J = 6.6 Hz, 1H), 6.10 (d, J = 2.6 Hz, 1H), 5.55-5.52 (m, 3H), 3.72-3.70 |
| | (m, 5H), 3.18 (t, J = 4.8 Hz, 4H), 2.80 (t, J = 11.6 Hz, 3H), 2.31 (s, 2H), 2.19 (s, 2H), 2.05 (d, J = |
| | 6.1 Hz, 2H), 1.87-1.72 (m, 3H), 1.66 (s, 2H), 1.52 (d, J = 12.3 Hz, 2H), 1.13 (t, J = 7.3 Hz, 3H) |
| 365 | ¹H NMR (400 MHz, DMSO-d₆) δ 10.93 (s, 1H), 8.04 (d, J = 2.4 Hz, 1H), 7.84 (d, J = 8.6 Hz, |
| | 1H), 7.66-7.54 (m, 2H), 7.03 (s, 2H), 6.96 (d, J = 8.6 Hz, 1H), 6.43 (d, J = 2.4 Hz, 1H), 6.08 (s, |
| | 1H), 5.65-5.48 (m, 1H), 3.67 (s, 2H), 3.30-3.20(m, 2H), 3.17 (d, J = 12.7 Hz, 4H), 2.31 (s, 1H), |
| | 2.17 (s, 2H), 2.06 (s, 2H), 1.81 (d, J = 12.3 Hz, 2H), 1.66 (s, 1H), 1.52 (d, J = 12.4 Hz, 2H), 1.26- |
| | 1.15 (m, 4H), 1.09-0.96 (m, 4H). |
| 366 | ¹H NMR (400 MHz, DMSO-d₆) δ 10.91 (s, 1H), 8.13 (d, J = 2.4 Hz, 1H), 8.02 (d, J = 8.1 Hz, |
| | 1H), 7.76-7.58 (m, 3H), 7.48 (s, 1H), 7.28 (d, J = 7.1 Hz, 1H), 7.20 (d, J = 7.9 Hz, 1H), 6.51 (d, J = |
| | 2.4 Hz, 1H), 6.03 (d, J = 16.1 Hz, 1H), 4.10 (s, 2H), 3.94 (s, 2H), 3.38 (t, J = 7.2 Hz, 1H), 3.24 |
| | (s, 2H), 3.04-2.94 (m, 4H), 2.78 (d, J = 7.3 Hz, 1H), 2.31 (s, 2H), 2.26 (d, J = 11.9 Hz, 1H), 2.19 |
| | (s, 1H), 1.81 (d, J = 10.5 Hz, 1H), 1.66 (d, J = 13.3 Hz, 5H), 1.23 (d, J = 7.7 Hz, 4H), 1.14 (d, J = |
| | 7.4 Hz, 1H). |
| 367 | ¹H NMR (400 MHz, DMSO-d₆) δ 10.67 (s, 1H), 8.11 (d, J = 2.4 Hz, 1H), 8.06 (d, J = 8.0 Hz, |
| | 1H), 7.72-7.61 (m, 2H), 7.59 (d, J = 1.7 Hz, 1H), 7.28 (t, J = 6.7 Hz, 1H), 6.50 (d, J = 2.4 Hz, |
| | 1H), 5.74-5.41 (m, 2H), 3.72 (s, 2H), 3.45-3.35 (m, 1H), 2.81 (t, J = 11.9 Hz, 2H), 2.49-2.43 (m, |
| | 4H), 2.35-2.23 (m, 3H), 2.20-2.14 (m, 2H), 2.08-2.01 (m, 2H), 1.86-1.73 (m, 3H), 1.71-1.61 (m, |
| | 1H), 1.55-1.48 (m, 2H), 1.31-1.19 (m, 3H), 1.13 (t, J = 7.3 Hz, 3H). |
| 368 | ¹H NMR (400 MHz, DMSO-d₆) δ 12.80 (s, 1H), 8.38-8.32 (m, 2H), 7.98 (d, J = 1.5 Hz, 1H), 7.81 |
| | (s, 1H), 7.73 (dd, J = 8.3, 1.2 Hz, 1H), 7.62 (dd, J = 8.3, 1.2 Hz, 1H), 7.05 (dd, J = 2.6, 1.2 Hz, |
| | 1H), 6.75 (t, J = 6.2 Hz, 1H), 5.52 (d, J = 11.8 Hz, 1H), 5.39 (d, J = 11.8 Hz, 1H), 4.38 (d, J = 6.4 |
| | Hz, 2H), 4.18 (d, J = 4.6 Hz, 2H), 3.46-3.36 (m, 4H), 3.18 (d, J = 11.5 Hz, 2H), 3.09 (t, J = 12.0 |
| | Hz, 2H), 3.04-2.93 (m, 2H), 1.71 (d, J = 12.3 Hz, 2H), 1.35 (s, 3H), 1.18 (t, J = 4.3 Hz, 2H), 1.14- |
| | 1.12 (m, 4H). |
| 369 | ¹H NMR (400 MHz, DMSO-d₆) δ 12.59 (s, 1H), 8.34 (d, J = 8.2 Hz, 1H), 7.94 (dd, J = 10.1, 2.1 |
| | Hz, 2H), 7.80 (dd, J = 8.2, 1.8 Hz, 1H), 7.67 (s, 1H), 7.62-7.42 (m, 2H), 5.84 (d, J = 2.5 Hz, 1H), |
| | 5.61-5.35 (m, 2H), 4.43 (s, 2H), 4.18 (d, J = 4.6 Hz, 2H), 3.50-3.38 (m, 2H), 3.31-3.21 (m, 4H), |
| | 3.21-3.02 (m, 4H), 2.32 (d, J = 12.0 Hz, 1H), 2.00-1.85 (m, 4H), 1.78-1.64 (m, 2H), 1.35 (s, 3H), |
| | 1.29 (s, 1H), 1.25-1.08 (m, 3H). |
| 370 | ¹H NMR (400 MHz, DMSO-d₆) δ 10.87 (s, 1H), 8.02 (d, J = 8.0 Hz, 1H), 7.95 (d, J = 2.4 Hz, |
| | 1H), 7.69 (d, J = 5.6 Hz, 2H), 7.66-7.61 (m, 1H), 7.61-7.52 (m, 2H), 6.02 (d, J = 16.0 Hz, 1H), |
| | 5.84 (d, J = 2.5 Hz, 1H), 5.54 (d, J = 15.6 Hz, 1H), 4.10 (s, 2H), 3.93 (s, 2H), 3.48-3.31 (m, 1H), |
| | 3.30-3.14 (m, 5H), 2.98 (t, J = 11.5 Hz, 3H), 2.32 (d, J = 12.4 Hz, 1H), 2.24-2.10 (m, 2H), 1.98- |
| | 1.88 (m, 4H), 1.63 (d, J = 12.3 Hz, 2H), 1.22 (s, 3H), 1.14 (t, J = 7.3 Hz, 3H). |
| 371 | ¹H NMR (400 MHz, DMSO-d6) δ 10.63 (s, 1H), 8.06 (d, J = 8.0 Hz, 1H), 7.93 (d, J = 2.5 Hz, |
| | 1H), 7.69-7.61 (m, 1H), 7.59 (d, J = 3.8 Hz, 1H), 7.55-7.46 (m, 1H), 5.84 (d, J = 2.6 Hz, 2H), |
| | 5.66-5.35 (m, 1H), 3.71 (s, 2H), 3.45-3.35 (m, 5H), 3.26 (d, J = 6.4 Hz, 4H), 2.81 (t, J = 11.6 Hz, |
| | 2H), 2.32 (d, J = 11.7 Hz, 2H), 2.19 (s, 2H), 2.06 (d, J = 7.2 Hz, 2H), 1.98-1.88 (m, 4H), 1.78 (t, J = |
| | 12.0 Hz, 1H), 1.66 (s, 1H), 1.52 (d, J = 12.2 Hz, 2H), 1.13 (t, J = 7.4 Hz, 3H). |
| 373 | ¹H NMR (400 MHz, DMSO-d₆) δ 10.75 (s, 1H), 8.12 (d, J = 0.8 Hz, 1H), 8.02 (d, J = 8.1 Hz, |
| | 1H), 7.76 (d, J = 0.8 Hz, 1H), 7.67 (d, J = 1.7 Hz, 1H), 7.64 (dd, J = 8.1, 1.6 Hz, 1H), 7.56 (d, J = |
| | 8.0 Hz, 1H), 7.50 (d, J = 8.0 Hz, 1H), 6.32 (t, J = 5.3 Hz, 1H), 6.12-5.91 (m, 2H), 5.61-5.48 (m, |
| | 1H), 5.08-4.87 (m, 1H), 4.11 (s, 2H), 3.84 (s, 2H), 3.76-3.68 (m, 2H), 3.48-3.41 (m, 2H), 3.37 (d, |
| | J = 7.4 Hz, 2H), 3.25 (d, J = 12.1 Hz, 2H), 2.98 (t, J = 11.7 Hz, 2H), 2.73-2.63 (m, 1H), 2.34 (s, |
| | 2H), 2.24 (s, 1H), 1.62 (d, J = 12.3 Hz, 2H), 1.22 (s, 3H), 1.14 (t, J = 7.3 Hz, 3H). |
| 374 | ¹H NMR (400 MHz, DMSO-d₆) δ 8.34 (d, J = 8.3 Hz, 1H), 8.06 (d, J = 2.4 Hz, 1H), 7.97 (d, J = |
| | 1.9 Hz, 1H), 7.80 (dd, J = 8.3, 1.8 Hz, 1H), 7.66 (d, J = 8.3 Hz, 1H), 7.57 (d, J = 8.3 Hz, 1H), 7.33 |
| | (d, J = 6.3 Hz, 1H), 6.43 (d, J = 2.5 Hz, 1H), 6.11 (s, 1H), 5.53-5.51 (m, 1H), 5.40 (d, J = 6.9 Hz, |
| | 1H), 4.42 (s, 2H), 4.18 (s, 2H), 3.41 (q, J = 7.4 Hz, 2H), 3.18 (d, J = 10.4 Hz, 2H), 3.08 (d, J = |
| | 11.9 Hz, 2H), 1.71 (d, J = 12.3 Hz, 2H), 1.57 (s, 2H), 1.35 (s, 2H), 1.33-1.27 (m, 2H), 1.14 (dd, J = |
| | 7.3 Hz, 3H), 1.03 (dd, J = 14.9, 2.7 Hz, 2H), 0.94 (t, J = 7.3 Hz, 2H). |
| 375 | ¹H NMR (400 MHz, DMSO-d₆) δ 8.10-8.02 (m, 1H), 7.67-7.56 (m, 2H), 7.25-7.20 (m, 1H), 7.14 |
| | (t, J = 6.5 Hz, 1H), 6.44 (d, J = 2.4 Hz, 1H), 5.65-5.46 (m, 2H), 5.33 (t, J = 4.7 Hz, 1H), 4.23 (t, J = |
| | 6.6 Hz, 2H), 3.69 (s, 2H), 3.38 (t, J = 7.3 Hz, 1H), 3.27 (s, 1H), 2.94 (s, 2H), 2.81 (t, J = 11.5 |
| | Hz, 1H), 2.31 (s, 1H), 2.18 (s, 1H), 2.07-1.96 (m, 3H), 1.79 (d, J = 11.4 Hz, 1H), 1.65 (s, 1H), |
| | 1.56-1.44 (m, 2H), 1.24 (s, 4H), 1.17-0.97 (m, 3H), 0.96-0.82 (m, 2H). |
| 376 | ¹H NMR (400 MHz, DMSO) δ 12.61 (s, 1H), 10.27 (s, 1H), 8.16-7.93 (m, 2H), 7.60 (q, J = 8.3 |
| | Hz, 2H), 7.25 (d, J = 3.3 Hz, 2H), 7.12 (dd, J = 8.6, 2.2 Hz, 1H), 6.42 (d, J = 2.4 Hz, 1H), 6.09 (s, |
| | 1H), 5.50 (s, 1H), 5.42-5.34 (m, 1H), 4.41 (s, 2H), 4.17 (s, 2H), 3.21 (q, J = 7.2 Hz, 3H), 3.12 (d, |
| | J = 11.4 Hz, 2H), 2.87 (t, J = 12.1 Hz, 3H), 1.71 (d, J = 12.3 Hz, 2H), 1.32 (d, J = 2.5 Hz, 3H), |
| | 1.28-1.14 (m, 5H), 0.95-0.79 (m, 2H) |
| 377 | ¹H NMR (400 MHz, DMSO-d₆) δ 10.97 (s, 1H), 8.39 (d, J = 2.5 Hz, 1H), 8.01 (d, J = 8.1 Hz, |
| | 1H), 7.75 (d, J = 8.3 Hz, 1H), 7.68 (d, J = 1.7 Hz, 1H), 7.63 (dd, J = 8.2, 1.5 Hz, 2H), 7.05 (d, J = |
| | 2.5 Hz, 1H), 6.90 (t, J = 5.4 Hz, 1H), 6.00 (d, J = 15.7 Hz, 1H), 5.57 (d, J = 15.5 Hz, 1H), 4.09 (s, |
| | 2H), 3.88 (s, 2H), 3.39 (d, J = 7.3 Hz, 4H), 3.26 (d, J = 12.0 Hz, 3H), 3.01-2.96 (m, 3H), 2.30 (s, |
| | 1H), 2.20 (t, J = 11.2 Hz, 2H), 1.64 (d, J = 12.3 Hz, 2H), 1.26-1.06 (m, 6H). |
| 378 | ¹H NMR (400 MHz, DMSO-d₆) δ 10.75 (s, 1H), 8.36 (dd, J = 2.5, 1.1 Hz, 1H), 8.06 (dd, J = 8.2, |
| | 1.1 Hz, 1H), 7.73-7.61 (m, 3H), 7.59 (t, J = 1.5 Hz, 1H), 7.06 (dd, J = 2.5, 1.1 Hz, 1H), 6.54 (t, J = |
| | 6.5 Hz, 1H), 5.66-5.46 (m, 2H), 3.66 (s, 2H), 3.42-3.30 (m, 2H), 3.33 (s, 2H), 3.03-2.92 (m, |
| | 1H), 2.82 (t, J = 11.6 Hz, 2H), 2.17 (s, 2H), 2.06 (dd, J = 7.3, 3.6 Hz, 2H), 1.79 (q, J = 11.3 Hz, |
| | 2H), 1.67 (d, J = 12.6 Hz, 1H), 1.53 (d, J = 12.2 Hz, 2H), 1.26-1.16 (m, 2H), 1.14-1.11 (m, 5H). |
| 379 | ¹H NMR (400 MHz, DMSO-d₆) δ 10.67 (s, 1H), 8.06 (d, J = 8.1 Hz, 1H), 7.96 (s, 1H), 7.65 (d, J = |
| | 1.8 Hz, 1H), 7.64-7.61 (m, 3H), 7.59 (d, J = 1.6 Hz, 1H), 7.15 (d, J = 6.7 Hz, 1H), 5.60-5.56 (m, |
| | 1H), 5.54-5.43 (m, 1H), 3.69 (d, J = 6.0 Hz, 2H), 3.37 (q, J = 7.4 Hz, 2H), 3.28 (d, J = 11.2 Hz, |
| | 2H), 2.86-2.75 (m, 2H), 2.61 (d, J = 52.7 Hz, 1H), 2.48-2.27 (m, 2H), 2.18 (s, 2H), 2.09-2.01 (m, |
| | 2H), 1.86-1.60 (m, 1H), 1.52 (d, J = 12.3 Hz, 2H), 1.24 (s, 1H), 1.13 (t, J = 7.3 Hz, 3H), 0.93-0.79 |
| | (m, 4H). |
| 380 | ¹H NMR (400 MHz, DMSO-d₆) δ 10.68 (s, 1H), 8.11 (s, 1H), 8.06 (d, J = 8.0 Hz, 1H), 7.70 (d, J = |
| | 4.2 Hz, 2H), 7.64 (dd, J = 8.2, 2.2 Hz, 2H), 7.59 (d, J = 1.7 Hz, 1H), 7.25 (d, J = 6.8 Hz, 1H), |
| | 5.69-5.40 (m, 2H), 3.71 (s, 3H), 3.37 (d, J = 7.4 Hz, 1H), 2.81 (d, J = 11.7 Hz, 2H), 2.51 (d, J = |
| | 1.1 Hz, 2H), 2.49 (s, 2H), 2.32 (d, J = 9.2 Hz, 1H), 2.19 (s, 6H), 2.05 (s, 2H), 1.79 (d, J = 12.4 |
| | Hz, 1H), 1.52 (d, J = 12.3 Hz, 2H), 1.24 (s, 1H), 1.13 (d, J = 7.3 Hz, 3H), 0.78 (dd, J = 11.0, 2.3 |
| | Hz, 4H). |
| 381 | ¹H NMR (400 MHz, DMSO) δ 11.17 (s, 1H), 10.21 (s, 1H), 8.07 (d, J = 2.5 Hz, 1H), 7.86 (d, J = |
| | 8.5 Hz, 1H), 7.62 (q, J = 8.3 Hz, 2H), 7.37 (t, J = 5.3 Hz, 1H), 7.14 (s, 1H), 7.03 (d, J = 8.7 Hz, |
| | 1H), 6.42 (d, J = 2.4 Hz, 1H), 6.04-6.02 (m, 2H), 5.54 (d, J = 15.8 Hz, 1H), 4.12 (s, 2H), 3.91 (s, |
| | 2H), 3.50-3.20 (m, 2H), 2.80 (t, J = 12.1 Hz, 2H), 2.34-2.22 (m, 2H), 1.65 (d, J = 12.6 Hz, 2H), |
| | 1.28-1.19 (m, 6H), 1.18-1.02 (m, 3H), 0.95-0.75 (m, 3H) |
| 382 | ¹H NMR (400 MHz, DMSO-d₆) δ 12.73 (s, 1H), 10.25 (s, 1H), 8.32 (d, J = 2.5 Hz, 1H), 8.09 (d, J = |
| | 8.7 Hz, 1H), 7.68 (d, J = 8.4 Hz, 1H), 7.63 (d, J = 8.3 Hz, 1H), 7.27 (d, J = 2.2 Hz, 1H), 7.14- |
| | 7.11 (m, 1H), 7.04 (d, J = 2.5 Hz, 1H), 6.70 (t, J = 6.1 Hz, 1H), 5.52-5.49 (m, 1H), 5.41-5.35 (m, |
| | 1H), 4.38 (s, 2H), 4.20-4.14 (m, 2H), 3.23 (q, J = 7.3 Hz, 2H), 3.13 (d, J = 11.5 Hz, 2H), 3.04- |
| | 2.93 (m, 1H), 2.87 (t, J = 12.1 Hz, 2H), 2.69-2.65 (m, 1H), 2.31 (s, 1H), 1.71 (d, J = 12.3 Hz, 2H), |
| | 1.31 (s, 3H), 1.25-1.18 (m, 4H), 1.12 (dd, J = 7.7, 2.6 Hz, 3H). |
| 383 | ¹H NMR (400 MHz, DMSO-d₆) δ 11.24 (s, 1H), 10.20 (s, 1H), 8.37 (d, J = 2.4 Hz, 1H), 7.88 (d, J = |
| | 8.5 Hz, 1H), 7.72 (d, J = 8.3 Hz, 1H), 7.65 (d, J = 8.3 Hz, 1H), 7.15 (d, J = 2.2 Hz, 1H), 7.05- |
| | 7.02 (m, 2H), 6.86 (t, J = 5.3 Hz, 1H), 6.01 (d, J = 15.9 Hz, 1H), 5.57 (d, J = 15.8 Hz, 1H), 4.12 |
| | (s, 2H), 3.87 (s, 2H), 3.27-3.11 (m, 4H), 3.00-2.97 (m, 1H), 2.86-2.75 (m, 2H), 2.33-2.22 (m, |
| | 2H), 1.66 (d, J = 12.1 Hz, 2H), 1.20 (dd, J = 13.8, 6.5 Hz, 8H), 1.13-1.06 (m, 2H). |
| 384 | ¹H NMR (400 MHz, DMSO-d₆) δ 10.95 (s, 1H), 10.23-10.18 (m, 1H), 8.34 (d, J = 2.5 Hz, 1H), |
| | 7.89 (d, J = 8.5 Hz, 1H), 7.71-7.61 (m, 2H), 7.09 (d, J = 2.1 Hz, 1H), 7.07-6.99 (m, 2H), 6.48 (t, J = |
| | 6.6 Hz, 1H), 5.58-5.54 (m, 2H), 3.64 (q, J = 6.1 Hz, 2H), 3.26-3.15 (m, 4H), 2.98-2.94 (m, 1H), |
| | 2.68 (t, J = 11.6 Hz, 2H), 2.20-2.11 (m, 2H), 2.10-2.02 (m, 2H), 1.87-1.74 (m, 2H), 1.68 (d, J = |
| | 11.9 Hz, 1H), 1.53 (d, J = 12.2 Hz, 2H), 1.25-1.14 (m, 5H), 1.14-1.11 (m, 2H). |
| 385 | ¹H NMR (400 MHz, DMSO) δ 10.69 (s, 1H), 8.39 (s, 1H), 8.14 (s, 1H), 8.06 (d, J = 8.0 Hz, 1H), |
| | 7.68-7.61 (m, 2H), 7.59 (d, J = 1.7 Hz, 1H), 6.90 (t, J = 6.7 Hz, 1H), 5.59-5.55 (m, 1H), 5.54-5.45 |
| | (m, 1H), 4.25 (s, 2H), 4.02 (dd, J = 6.1, 4.2 Hz, 2H), 3.80 (dd, J = 6.1, 4.3 Hz, 2H), 3.69 (d, J = |
| | 8.8 Hz, 2H), 3.38 (t, J = 7.3 Hz, 3H), 2.81 (t, J = 11.6 Hz, 2H), 2.18 (d, J = 9.1 Hz, 2H), 2.05 (dd, |
| | J = 7.4, 3.6 Hz, 2H), 1.86-1.73 (m, 2H), 1.67 (d, J = 12.6 Hz, 1H), 1.53 (d, J = 12.3 Hz, 2H), 1.24 |
| | (s, 2H), 1.13 (t, J = 7.3 Hz, 3H). |
| 386 | ¹H NMR (400 MHz, DMSO-d₆) δ 10.92 (s, 1H), 8.37 (s, 1H), 8.13 (s, 1H), 7.87 (d, J = 8.5 Hz, |
| | 1H), 7.69-7.56 (m, 2H), 7.07 (d, J = 2.1 Hz, 1H), 7.00 (dd, J = 8.6, 2.1 Hz, 1H), 6.83 (t, J = 6.8 |
| | Hz, 1H), 5.55 (m, 2H), 4.25 (s, 2H), 4.01 (dd, J = 6.3, 4.1 Hz, 2H), 3.80 (t, J = 5.1 Hz, 2H), 3.67 |
| | (d, J = 9.1 Hz, 2H), 3.18 (t, J = 7.2 Hz, 4H), 2.67 (t, J = 11.5 Hz, 2H), 2.16 (s, 2H), 2.06 (m, 2H), |
| | 1.79 (t, J = 11.4 Hz, 2H), 1.66 (s, 2H), 1.53 (d, J = 12.2 Hz, 2H), 1.20 (t, J = 7.3 Hz, 3H) |
| 387 | ¹H NMR (300 MHz, DMSO-d₆) δ 11.18 (s, 1H), 10.19 (s, 1H), 8.12 (d, J = 2.4 Hz, 1H), 7.88 (d, J = |
| | 8.5 Hz, 1H), 7.60 (d, J = 1.3 Hz, 2H), 7.15 (d, J = 2.1 Hz, 1H), 7.04 (dd, J = 8.6, 2.0 Hz, 1H), |
| | 6.78 (t, J = 5.2 Hz, 1H), 6.68 (d, J = 2.4 Hz, 1H), 5.98 (d, J = 15.8 Hz, 1H), 5.54 (d, J = 15.9 Hz, |
| | 1H), 4.13 (s, 2H), 3.86 (s, 2H), 3.29-3.10 (m, 4H), 2.80 (t, J = 11.9 Hz, 2H), 2.27 (t, J = 11.8 |
| | Hz, 2H), 1.65 (d, J = 12.1 Hz, 2H), 1.27-1.16 (m, 6H). |
| 388 | ¹H NMR (300 MHz, DMSO-d₆) δ 12.61 (s, 1H), 10.25 (s, 1H), 8.13-8.03 (m, 2H), 7.64-7.49 |
| | (m, 2H), 7.28 (d, J = 2.1 Hz, 1H), 7.14 (dd, J = 8.6, 2.1 Hz, 1H), 6.71-6.57 (m, 2H), 5.56-5.30 |
| | (m, 2H), 4.34 (t, J = 6.1 Hz, 2H), 4.21-4.13 (m, 2H), 3.30-3.07 (m, 4H), 2.94-2.80 (m, 2H), |
| | 2.55 (s, 1H), 2.48 (s, 1H), 1.68 (d, J = 12.3 Hz, 2H), 1.31 (s, 3H), 1.22 (t, J = 7.3 Hz, 3H). |
| 391 | ¹H NMR (300 MHz, DMSO-d₆) δ 10.92 (s, 1H), 10.21 (s, 1H), 8.08 (d, J = 2.5 Hz, 1H), 7.89 (d, J = |
| | 8.5 Hz, 1H), 7.64 (d, J = 8.3 Hz, 1H), 7.52 (d, J = 8.3 Hz, 1H), 7.10 (d, J = 2.1 Hz, 1H), 7.03 |
| | (dd, J = 8.6, 2.0 Hz, 1H), 6.68 (d, J = 2.4 Hz, 1H), 6.40 (t, J = 6.6 Hz, 1H), 5.67-5.43 (m, 2H), |
| | 3.62 (s, 2H), 3.21 (dd, J = 9.2, 6.3 Hz, 4H), 2.68 (t, J = 11.5 Hz, 2H), 2.17 (d, J = 8.9 Hz, 2H), |
| | 2.16-2.01 (m, 2H), 1.92-1.73 (m, 2H), 1.68 (d, J = 12.2 Hz, 1H), 1.53 (d, J = 12.1 Hz, 2H), |
| | 1.21 (t, J = 7.3 Hz, 3H). |
| 393 | ¹H NMR (300 MHz, DMSO-d₆) δ 10.90 (s, 1H), 10.20 (s, 1H), 8.37 (d, J = 0.7 Hz, 1H), 7.93- |
| | 7.85 (m, 2H), 7.70-7.52 (m, 2H), 7.13-6.98 (m, 2H), 6.61 (t, J = 6.7 Hz, 1H), 5.67-5.43 (m, |
| | 2H), 3.63 (s, 2H), 3.21 (q, J = 8.1 Hz, 4H), 2.67 (t, J = 11.5 Hz, 2H), 2.17 (d, J = 10.4 Hz, 2H), |
| | 2.06 (dd, J = 8.1, 4.7 Hz, 2H), 1.90-1.45 (m, 5H), 1.21 (t, J = 7.3 Hz, 3H). |
| 395 | ¹H NMR (400 MHz, DMSO-d₆) δ 12.68 (s, 1H), 8.40 (s, 1H), 8.35 (d, J = 8.2 Hz, 1H), 8.13 (s, |
| | 1H), 7.98 (d, J = 1.8 Hz, 1H), 7.81 (dd, J = 8.2, 1.7 Hz, 1H), 7.68 (d, J = 8.4 Hz, 1H), 7.60 (d, J = |
| | 8.0 Hz, 1H), 5.51 (d, J = 11.6 Hz, 1H), 5.43-5.33 (m, 1H), 4.39 (s, 2H), 4.26 (s, 2H), 4.18 (d, J = |
| | 4.6 Hz, 2H), 4.02 (dd, J = 6.1, 4.4 Hz, 2H), 3.80 (t, J = 5.3 Hz, 2H), 3.41 (q, J = 7.3 Hz, 2H), 3.18 |
| | (d, J = 11.7 Hz, 2H), 3.09 (t, J = 12.0 Hz, 2H), 2.31 (s, 1H), 2.08 (d, J = 1.3 Hz, 1H), 1.69 (d, J = |
| | 12.4 Hz, 2H), 1.35 (s, 3H), 1.23-1.16 (m, 1H), 1.15 (dd, J = 7.9, 6.6 Hz, 3H) |
| 396 | ¹H NMR (400 MHz, DMSO-d₆) δ 10.88 (s, 1H), 8.43 (s, 1H), 8.13 (s, 1H), 8.01 (d, J = 8.1 Hz, |
| | 1H), 8.00-7.66 (m, 3H), 7.23 (t, J = 5.5 Hz, 1H), 5.98 (d, J = 15.5 Hz, 1H), 5.54 (d, J = 15.9 Hz, |
| | 1H), 4.26 (s, 2H), 4.10 (s, 2H), 4.05-3.97 (m, 2H), 3.90 (s, 2H), 3.80 (t, J = 5.3 Hz, 2H), 3.43- |
| | 3.35 (m, 4H), 2.99 (t, J = 11.5 Hz, 2H), 2.20 (t, J = 11.7 Hz, 3H), 1.63 (d, J = 12.3 Hz, 2H), 1.22 |
| | (s, 3H), 1.14 (t, J = 7.3 Hz, 3H) |
| 397 | ¹H NMR (400 MHz, DMSO-d₆) δ 10.72 (s, 1H), 8.51 (d, J = 0.7 Hz, 1H), 8.13-8.03 (m, 2H), 7.70 |
| | (d, J = 8.3 Hz, 1H), 7.67-7.61 (m, 2H), 7.59 (d, J = 1.7 Hz, 1H), 6.75 (t, J = 6.6 Hz, 1H), 5.65- |
| | 5.44 (m, 2H), 3.73 (t, J = 6.7 Hz, 2H), 3.67 (s, 2H), 3.48 (t, J = 6.8 Hz, 2H), 3.37 (q, J = 7.3 Hz, |
| | 4H), 2.81 (t, J = 11.6 Hz, 2H), 2.17 (s, 2H), 2.06-2.04 (m, 2H), 2.00-1.73 (m, 6H), 1.67 (d, J = |
| | 12.2 Hz, 1H), 1.53 (d, J = 11.7 Hz, 2H), 1.13 (t, J = 7.3 Hz, 3H). |
| 398 | ¹H NMR (400 MHz, DMSO-d₆) δ 10.92 (s, 1H), 10.20 (s, 1H), 8.48 (d, J = 0.7 Hz, 1H), 8.10 (d, J = |
| | 0.7 Hz, 1H), 7.89 (d, J = 8.6 Hz, 1H), 7.65 (s, 2H), 7.09 (d, J = 2.1 Hz, 1H), 7.02 (dd, J = 8.5, |
| | 2.1 Hz, 1H), 6.68 (t, J = 6.7 Hz, 1H), 5.66-5.46 (m, 2H), 3.73 (t, J = 6.7 Hz, 2H), 3.65 (s, 2H), |
| | 3.47 (t, J = 6.8 Hz, 2H), 3.20 (dd, J = 9.2, 5.9 Hz, 4H), 2.68 (t, J = 11.6 Hz, 2H), 2.16 (d, J = 4.1 |
| | Hz, 2H), 2.05 (dd, J = 7.2, 3.7 Hz, 2H), 1.99-1.86 (m, 2H), 1.81-1.84 (m, 4H), 1.67 (d, J = 13.4 |
| | Hz, 1H), 1.53 (d, J = 12.2 Hz, 2H), 1.20 (t, J = 7.3 Hz, 3H). |
| 399 | ¹H NMR (400 MHz, DMSO-d₆) δ 10.74 (s, 1H), 8.78 (s, 1H), 8.18 (s, 1H), 8.06 (d, J = 8.1 Hz, |
| | 1H), 7.71 (d, J = 8.3 Hz, 1H), 7.67-7.63 (m, 2H), 7.60 (d, J = 1.7 Hz, 1H), 6.66 (t, J = 6.6 Hz, |
| | 1H), 5.72-5.42 (m, 2H), 3.66 (s, 2H), 3.43-3.35 (m, 2H), 3.25-3.16 (m, 4H), 2.87-2.76 (m, 2H), |
| | 2.70-2.65 (m, 1H), 2.36-2.30 (m, 1H), 2.18 (s, 2H), 2.12-2.01 (m, 2H), 1.85-1.61 (m, 6H), 1.57- |
| | 1.49 (m, 2H), 1.14 (t, J = 7.3 Hz, 4H). |
| 400 | ¹H NMR (400 MHz, DMSO) δ 10.74 (s, 1H), 8.78 (s, 1H), 8.18 (s, 1H), 8.06 (d, J = 8.1 Hz, 1H), |
| | 7.71 (d, J = 8.3 Hz, 1H), 7.67-7.63 (m, 2H), 7.60 (d, J = 1.7 Hz, 1H), 6.66 (t, J = 6.6 Hz, 1H), |
| | 5.72-5.42 (m, 2H), 3.66 (s, 2H), 3.43-3.35 (m, 2H), 3.25-3.16 (m, 4H), 2.87-2.76 (m, 2H), 2.70- |
| | 2.65 (m, 1H), 2.36-2.30 (m, 1H), 2.18 (s, 2H), 2.12-2.01 (m, 2H), 1.85-1.61 (m, 7H), 1.57-1.49 |
| | (m, 2H), 1.14 (t, J = 7.3 Hz, 4H). |
| 401 | ¹H NMR (400 MHz, DMSO-d₆) δ 10.93 (s, 1H), 8.13 (d, J = 2.6 Hz, 1H), 7.87 (d, J = 8.6 Hz, |
| | 1H), 7.70-7.57 (m, 2H), 7.07 (d, J = 2.2 Hz, 1H), 7.00 (dd, J = 8.4, 2.0 Hz, 1H), 6.73 (t, J = 6.7 |
| | Hz, 1H), 6.67 (d, J = 2.5 Hz, 1H), 5.66-5.48 (m, 2H), 3.65 (s, 1H), 3.33-3.31(m, 4H), 3.19 (d, J = |
| | 7.9 Hz, 3H), 3.08 (d, J = 2.1 Hz, 2H), 2.71-2.60 (m, 2H), 2.16 (s, 2H), 2.07 (d, J = 7.9 Hz, 3H), |
| | 1.79 (t, J = 12.3 Hz, 3H), 1.67-1.65 (m, 2H), 1.57-1.46 (m, 3H), 1.19-1.16 (m, 3H). |
| 402 | ¹H NMR (400 MHz, DMSO-d₆) δ 12.59 (s, 1H), 10.24 (s, 1H), 8.20 (s, 1H), 8.08 (d, J = 8.6 Hz, |
| | 1H), 7.84 (s, 1H), 7.68-7.56 (m, 2H), 7.26 (s, 1H), 7.20-7.09 (m, 2H), 5.49 (s, 1H), 5.39 (s, 1H), |
| | 4.89 (dd, J = 8.4, 5.6 Hz, 2H), 4.65 (dd, J = 7.2, 5.7 Hz, 2H), 4.38 (s, 2H), 4.25 (t, J = 7.7 Hz, |
| | 1H), 4.17 (s, 2H), 3.23 (d, J = 7.2 Hz, 2H), 3.12 (d, J = 11.5 Hz, 2H), 2.87 (t, J = 11.7 Hz, 2H), |
| | 2.30 (s, 2H), 1.69 (d, J = 12.3 Hz, 2H), 1.31 (s, 3H), 1.21 (t, J = 7.3 Hz, 3H). |
| 403 | ¹H NMR (400 MHz, DMSO-d₆) δ 12.64 (s, 1H), 10.20 (s, 1H), 8.37 (s, 1H), 8.18-7.96 (m, |
| | 2H), 7.70-7.50 (m, 2H), 7.24 (d, J = 2.2 Hz, 1H), 7.11 (dd, J = 8.7, 2.1 Hz, 1H), 7.04 (t, J = 6.3 |
| | Hz, 1H), 5.50 (d, J = 11.2 Hz, 1H), 5.43-5.32 (m, 1H), 4.38 (s, 2H), 4.25 (s, 2H), 4.21-4.12 (m, |
| | 2H), 4.01 (dd, J = 6.1, 4.3 Hz, 2H), 3.80 (t, J = 5.2 Hz, 2H), 3.20 (q, J = 7.3 Hz, 2H), 3.12 (d, J = |
| | 11.7 Hz, 2H), 2.87 (t, J = 12.1 Hz, 2H), 1.69 (d, J = 12.3 Hz, 3H), 1.37-1.11 (m, 7H) |
| 404 | ¹H NMR (400 MHz, DMSO-d₆) δ 11.19 (s, 1H), 10.18 (s, 1H), 8.41 (s, 1H), 8.12 (s, 1H), 7.86 (d, |
| | J = 8.5 Hz, 1H), 7.77-7.58 (m, 2H), 7.25-7.06 (m, 2H), 7.02 (dd, J = 8.6, 2.0 Hz, 1H), 5.99 (d, J = |
| | 15.7 Hz, 1H), 5.54 (d, J = 15.8 Hz, 1H), 4.25 (s, 2H), 4.13 (s, 2H), 4.02 (dd, J = 6.1, 4.2 Hz, 2H), |
| | 3.89 (s, 2H), 3.80 (dd, J = 6.2, 4.3 Hz, 2H), 3.18-2.85 (m, 4H), 2.80 (t, J = 11.9 Hz, 2H), 2.29 (d, |
| | J = 9.5 Hz, 2H), 1.65 (d, J = 12.0 Hz, 2H), 1.31-1.11 (m, 6H) |
| 405 | ¹H NMR (400 MHz, DMSO-d₆) δ 10.90 (s, 1H), 10.19 (s, 1H), 8.17 (d, J = 0.7 Hz, 1H), 7.97 (d, J = |
| | 0.7 Hz, 1H), 7.88 (d, J = 8.5 Hz, 1H), 7.68-7.57 (m, 2H), 7.08 (d, J = 2.1 Hz, 1H), 7.01 (dd, J = |
| | 8.6, 2.0 Hz, 1H), 6.86 (t, J = 6.7 Hz, 1H), 5.66-5.45 (m, 2H), 4.55-4.47 (m, 2H), 4.01-3.93 (m, |
| | 2H), 3.68-3.61 (m, 2H), 3.20 (q, J = 7.2 Hz, 4H), 2.72-2.62 (m, 2H), 2.16 (t, J = 5.8 Hz, 2H), |
| | 2.10-2.02 (m, 2H), 1.79 (dd, J = 13.4, 9.9 Hz, 2H), 1.67 (d, J = 11.8 Hz, 1H), 1.53 (d, J = 12.2 Hz, |
| | 2H), 1.20 (t, J = 7.3 Hz, 3H). |
| 445 | ¹H NMR (400 MHz, DMSO-d₆) δ 10.70 (s, 1H), 8.21 (d, J = 0.9 Hz, 1H), 8.06 (d, J = 8.1 Hz, |
| | 1H), 7.84 (d, J = 0.8 Hz, 1H), 7.65 (d, J = 6.0 Hz, 3H), 7.59 (d, J = 1.7 Hz, 1H), 6.87 (s, 1H), |
| | 5.55 (d, J = 25.1 Hz, 1H), 3.68 (s, 2H), 3.37 (q, J = 7.4 Hz, 2H), 3.29 (d, J = 10.4 Hz, 2H), 2.81 |
| | (t, J = 11.6 Hz, 3H), 2.32 (d, J = 9.9 Hz, 1H), 2.17 (s, 2H), 2.06 (s, 2H), 1.86-1.59 (m, 3H), 1.53 |
| | (d, J = 12.1 Hz, 2H), 1.48-1.38 (m, 2H), 1.14 (t, J = 7.4 Hz, 4H). |
| 462 | ¹H NMR (400 MHz, DMSO-d₆) δ 10.90 (s, 1H), 8.09 (d, J = 2.4 Hz, 1H), 8.01 (d, J = 8.1 Hz, |
| | 1H), 7.68 (dd, J = 5.0, 3.3 Hz, 2H), 7.64 (dd, J = 8.1, 1.7 Hz, 1H), 7.59 (d, J = 8.3 Hz, 1H), 7.42 |
| | (d, J = 5.4 Hz, 1H), 6.42 (d, J = 2.4 Hz, 1H), 6.09 (s, 1H), 6.01 (d, J = 15.6 Hz, 1H), 5.54 (d, J = |
| | 15.8 Hz, 1H), 4.10 (s, 2H), 3.92 (s, 2H), 3.37 (q, J = 7.9 Hz, 2H), 3.25 (d, J = 11.7 Hz, 2H), 2.98 |
| | (t, J = 11.5 Hz, 2H), 2.31 (d, J = 1.1 Hz, 1H), 2.20 (t, J = 11.3 Hz, 2H), 1.63 (d, J = 12.5 Hz, 2H), |
| | 1.57 (s, 1H), 1.31 (d, J = 7.3 Hz, 1H), 1.22 (s, 3H), 1.13 (d, J = 7.3 Hz, 3H), 0.94 (d, J = 7.3 Hz, |
| | 1H). |
| 463 | ¹H NMR (400 MHz, DMSO-d₆) δ 10.66 (s, 1H), 8.05 (d, J = 8.1 Hz, 1H), 7.93 (s, 1H), 7.70-7.57 |
| | (m, 5H), 7.14 (s, 1H), 5.95 (s, 1H), 5.59 (d, J = 12.4 Hz, 1H), 5.48 (d, J = 15.0 Hz, 1H), 4.29 (s, |
| | 1H), 4.04-3.97 (m, 1H), 3.88-3.75 (m, 3H), 3.69 (s, 4H), 2.81 (t, J = 11.8 Hz, 2H), 2.17 (s, 4H), |
| | 2.05 (s, 2H), 1.78 (d, J = 12.9 Hz, 2H), 1.66 (s, 1H), 1.52 (d, J = 12.4 Hz, 2H), 1.02 (s, 2H), 0.85 |
| | (s, 2H). |
| 464 | ¹H NMR (400 MHz, DMSO-d₆) δ 10.89 (s, 1H), 8.00 (d, J = 8.1 Hz, 1H), 7.98 (s, 1H), 7.70 (d, J = |
| | 8.2 Hz, 1H), 7.65 (dd, J = 8.1, 1.7 Hz, 1H), 7.63-7.55 (m, 2H), 7.42 (t, J = 5.4 Hz, 1H), 6.00 (s, |
| | 1H), 5.95 (d, J = 9.4 Hz, 1H), 5.53 (d, J = 16.1 Hz, 1H), 4.94 (s, 1H), 4.10 (s, 2H), 3.92 (s, 2H), |
| | 3.72 (s, 2H), 3.53 (t, J = 6.3 Hz, 2H), 2.98 (t, J = 11.9 Hz, 3H), 2.31 (s, 2H), 2.20 (t, J = 10.8 Hz, |
| | 2H), 1.63 (d, J = 12.4 Hz, 2H), 1.22 (s, 3H), 1.06-1.00 (m, 2H), 0.88-0.83 (m, 2H). |
| 465 | ¹H NMR (400 MHz, DMSO-d₆) δ 10.69 (s, 1H), 8.05 (d, J = 8.0 Hz, 1H), 7.89 (d, J = 2.6 Hz, |
| | 1H), 7.68-7.60 (m, 2H), 7.59 (d, J = 1.7 Hz, 1H), 7.53 (d, J = 8.3 Hz, 1H), 6.51 (d, J = 6.7 Hz, |
| | 1H), 5.95 (s, 1H), 5.64-5.42 (m, 2H), 3.65 (d, J = 6.1 Hz, 2H), 2.80 (d, J = 11.6 Hz, 2H), 2.18 (s, |
| | 2H), 2.09-2.02 (m, 2H), 1.78 (d, J = 11.9 Hz, 2H), 1.67 (d, J = 12.5 Hz, 1H), 1.52 (d, J = 12.2 Hz, |
| | 2H), 1.20-1.09 (m, 4H), 0.97-0.92 (m, 2H), 0.89 (dd, J = 5.9, 2.5 Hz, 2H), 0.77 (s, 3H). |
| 486 | ¹H NMR (400 MHz, DMSO-d₆) δ 10.69 (s, 1H), 8.19 (s, 1H), 8.06 (d, J = 8.1 Hz, 1H), 7.98 (s, |
| | 1H), 7.64 (d, J = 8.2 Hz, 3H), 7.59 (d, J = 1.9 Hz, 1H), 6.94 (t, J = 6.7 Hz, 1H), 5.63-5.56 (m, |
| | 1H), 5.54-5.44 (m, 1H), 4.51 (dd, J = 9.0, 7.0 Hz, 2H), 3.98 (dd, J = 9.0, 7.0 Hz, 2H), 3.68 (s, |
| | 2H), 3.38 (t, J = 7.3 Hz, 2H), 3.27 (s, 2H), 2.81 (t, J = 11.6 Hz, 2H), 2.17 (s, 2H), 2.05 (dd, J = |
| | 7.6, 3.6 Hz, 2H), 1.79 (q, J = 10.8 Hz, 2H), 1.67 (d, J = 12.4 Hz, 1H), 1.52 (d, J = 12.2 Hz, 2H), |
| | 1.13 (t, J = 7.3 Hz, 3H). |
| 487 | ¹H NMR (400 MHz, DMSO-d₆) δ 10.72 (s, 1H), 8.29 (s, 1H), 8.10 (s, 1H), 8.05 (d, J = 8.0 Hz, |
| | 1H), 7.69-7.61 (m, 4H), 6.94 (t, J = 6.7 Hz, 1H), 5.67-5.45 (m, 2H), 4.93 (d, J = 5.3 Hz, 1H), 3.77 |
| | (d, J = 7.3 Hz, 2H), 3.71 (d, J = 6.0 Hz, 1H), 3.53 (t, J = 6.2 Hz, 2H), 3.29 (d, J = 12.1 Hz, 2H), |
| | 2.81 (t, J = 11.7 Hz, 2H), 2.68 (s, 1H), 2.45 (d, J = 8.1 Hz, 2H), 2.32 (d, J = 9.4 Hz, 1H), 2.26- |
| | 2.10 (m, 4H), 2.06 (s, 2H), 1.90-1.73 (m, 2H), 1.67 (s, 1H), 1.53 (d, J = 12.2 Hz, 2H), 1.24 (s, |
| | 1H).. |
| 488 | ¹H NMR (400 MHz, DMSO-d₆) δ 10.96 (s, 1H), 8.26 (s, 1H), 8.08 (s, 1H), 7.86 (d, J = 8.6 Hz, |
| | 1H), 7.65 (d, J = 8.4 Hz, 1H), 7.59 (d, J = 8.3 Hz, 1H), 7.02 (s, 1H), 6.96 (d, J = 8.5 Hz, 1H), 6.84 |
| | (d, J = 6.9 Hz, 1H), 5.65-5.46 (m, 1H), 3.79-3.76 (m, 2H), 3.76-3.72 (m, 2H), 3.66 (s, 2H), 3.18 |
| | (d, J = 11.4 Hz, 3H), 2.72-2.64 (m, 2H), 2.46 (d, J = 8.0 Hz, 2H), 2.35-2.29 (m, 1H), 2.22-2.10 |
| | (m, 4H), 2.07 (d, J = 7.7 Hz, 2H), 1.80 (d, J = 11.9 Hz, 2H), 1.66 (s, 1H), 1.53 (d, J = 12.2 Hz, |
| | 2H), 1.24 (s, 2H), 0.85 (d, J = 7.3 Hz, 1H). |
| 489 | ¹H NMR (400 MHz, DMSO-d₆) δ 12.56 (s, 1H), 8.08 (t, J = 4.3 Hz, 2H), 7.63 (d, J = 8.3 Hz, 1H), |
| | 7.37 (d, J = 8.2 Hz, 1H), 7.18 (s, 1H), 7.09 (d, J = 7.8 Hz, 2H), 5.50 (d, J = 5.9 Hz, 3H), 3.98 (s, |
| | 2H), 3.76 (t, J = 6.5 Hz, 4H), 3.45 (s, 3H), 3.09 (s, 2H), 2.78 (t, J = 11.5 Hz, 2H), 2.45 (d, J = 8.2 |
| | Hz, 1H), 2.26 (s, 2H), 2.21-2.07 (m, 3H), 1.91 (t, J = 11.5 Hz, 2H), 1.81 (d, J = 12.7 Hz, 2H), |
| | 1.24 (s, 2H), 0.85 (d, J = 8.7 Hz, 2H). |
| 490 | ¹H NMR (400 MHz, DMSO-d₆) δ 12.58 (s, 1H), 8.37 (d, J = 8.3 Hz, 1H), 8.30 (s, 1H), 8.09 (s, |
| | 1H), 7.92 (d, J = 1.9 Hz, 1H), 7.83 (dd, J = 8.3, 1.8 Hz, 1H), 7.68 (d, J = 8.2 Hz, 1H), 7.37 (d, J = |
| | 8.2 Hz, 1H), 7.19 (t, J = 6.4 Hz, 1H), 5.51 (s, 2H), 4.93 (t, J = 5.4 Hz, 1H), 3.80-3.73 (m, 3H), |
| | 3.55 (d = 6.2 Hz, 2H), 3.47 (s, 3H), 3.30 (s, 1H), 3.17 (d, J = 11.2 Hz, 2H), 2.97 (d = 11.5 Hz, |
| | 2H), 2.31 (s, 1H), 2.27 (s, 2H), 2.16 (d = 15.7, 7.9 Hz, 4H), 1.94 (d, J = 12.1 Hz, 1H), 1.83 (d, J = |
| | 13.4 Hz, 2H), 1.24 (s, 2H).. |
| 560 | ¹H NMR (400 MHz, DMSO-d₆) δ 11.14 (s, 1H), 10.20 (s, 1H), 8.22 (s, 1H), 7.89-7.81 (m, 2H), |
| | 7.67 (d, J = 8.3 Hz, 1H), 7.61 (d, J = 8.3 Hz, 1H), 7.27 (t, J = 5.3 Hz, 1H), 7.13 (s, 1H), 7.03 (d, J = |
| | 8.6 Hz, 1H), 5.98 (d, J = 15.9 Hz, 1H), 5.53 (d, J = 15.7 Hz, 1H), 4.89 (dd, J = 8.5, 5.6 Hz, 2H), |
| | 4.65 (t, J = 6.4 Hz, 2H), 4.25 (p, J = 7.8 Hz, 1H), 4.12 (s, 2H), 3.89 (s, 2H), 3.23-3.12 (m, 4H), |
| | 2.79 (t, J = 12.3 Hz, 2H), 2.32-2.21 (m, 2H), 1.64 (d, J = 12.1 Hz, 2H), 1.20 (q, J = 5.6 Hz, 6H). |
| 561 | ¹H NMR (400 MHz, DMSO-d₆) δ 10.88 (s, 1H), 10.20 (s, 1H), 7.96 (d, J = 0.9 Hz, 1H), 7.88 (d, J = |
| | 8.5 Hz, 1H), 7.64 (d, J = 8.4 Hz, 1H), 7.60-7.56 (m, 1H), 7.09 (d, J = 2.1 Hz, 1H), 7.06-6.98 |
| | (m, 2H), 5.56 (d, J = 18.8 Hz, 2H), 4.82 (d, J = 1.9 Hz, 1H), 3.90-3.72 (m, 4H), 3.67 (s, 2H), 3.21 |
| | (q, J = 7.6 Hz, 3H), 2.67 (t, J = 11.6 Hz, 2H), 2.25-2.12 (m, 3H), 2.09-1.96 (m, 3H), 1.81 (d, J = |
| | 12.3 Hz, 2H), 1.66 (s, 1H), 1.53 (d, J = 12.4 Hz, 2H), 1.22 (q, J = 7.2 Hz, 4H), 0.85 (d, J = 7.2 |
| | Hz, 1H). |
| 562 | ¹H NMR (400 MHz, DMSO-d₆) δ 10.88 (s, 1H), 10.20 (s, 1H), 7.96 (s, 1H), 7.88 (d, J = 8.4 Hz, |
| | 1H), 7.64 (d, J = 8.5 Hz, 1H), 7.61-7.56 (m, 1H), 7.09 (d, J = 2.1 Hz, 1H), 7.07-6.98 (m, 2H), |
| | 5.55-5.52 (m, 2H), 4.85-4.79 (m, 1H), 3.92-3.80 (m, 3H), 3.76 (m, 1H), 3.67 (s, 2H), 3.21 (q, J = |
| | 7.8 Hz, 3H), 2.68 (t, J = 11.6 Hz, 2H), 2.25-2.13 (m, 3H), 2.06 (d, J = 6.5 Hz, 3H), 1.81 (q, J = |
| | 11.9 Hz, 2H), 1.66 (s, 1H), 1.53 (d, J = 12.3 Hz, 2H), 1.33-1.15 (m, 4H), 0.85 (d, J = 7.1 Hz, 1H). |
| 563 | ¹H NMR (400 MHz, DMSO-d₆) δ 10.67 (s, 1H), 8.06 (d, J = 8.0 Hz, 1H), 7.98 (d, J = 1.0 Hz, |
| | 1H), 7.72-7.50 (m, 4H), 7.11 (t, J = 6.7 Hz, 1H), 5.56-5.52 (m, 2H), 4.82 (s, 1H), 3.90-3.73 (m, |
| | 4H), 3.69 (s, 2H), 3.41-3.35 (m, 2H), 2.81 (t, J = 11.7 Hz, 2H), 2.17 (q, J = 7.7 Hz, 3H), 2.06 (t, J = |
| | 5.8 Hz, 3H), 1.80 (q, J = 11.9 Hz, 2H), 1.66 (s, 2H), 1.53 (d, J = 12.4 Hz, 2H), 1.24 (s, 2H), |
| | 1.14 (t, J = 7.3 Hz, 3H). |
| 564 | ¹H NMR (400 MHz, DMSO-d₆) δ 10.67 (s, 1H), 8.06 (d, J = 8.1 Hz, 1H), 7.98 (d, J = 0.9 Hz, |
| | 1H), 7.70-7.55 (m, 4H), 7.11 (t, J = 6.7 Hz, 1H), 5.65-5.46 (m, 2H), 5.05-4.82 (m, 1H), 3.87-3.81 |
| | (m, 2H), 3.80-3.77 (m, 1H), 3.69 (s, 2H), 3.38 (t, J = 7.3 Hz, 3H), 2.87-2.77 (m, 2H), 2.22-2.15 |
| | (m, 3H), 2.09-2.03 (m, 3H), 1.80 (d, J = 11.4 Hz, 2H), 1.77-1.60 (m, 2H), 1.52 (d, J = 12.4 Hz, |
| | 2H), 1.24 (s, 2H), 1.14 (t, J = 7.3 Hz, 3H). |
| 586 | ¹H NMR (400 MHz, DMSO) δ 10.73 (s, 1H), 8.80 (s, 1H), 8.23 (s, 1H), 8.06 (d, J = 8.0 Hz, 1H), |
| | 7.78-7.62 (m, 3H), 6.68 (t, J = 6.7 Hz, 1H), 5.53-5.50(m, 2H), 3.66 (s, 4H), 3.29 (s, 3H), 2.93- |
| | 2.77 (m, 4H), 2.21-2.12 (m, 2H), 2.03 (d, J = 19.4 Hz, 2H), 1.85-1.73 (m, 2H), 1.73-1.60 (m, 1H), |
| | 1.59-1.49 (m, 2H), 1.25 (d, J = 13.8 Hz, 2H), 1.17 (s, 1H), 1.09 (t, J = 7.3 Hz, 3H). |
| 587 | ¹H NMR (400 MHz, DMSO-d₆) δ 10.68 (s, 1H), 8.18-7.98 (m, 2H), 7.74 (s, 1H), 7.68-7.52 (m, |
| | 3H), 7.14 (t, J = 6.8 Hz, 1H), 5.66-5.42 (m, 2H), 5.33 (t, J = 4.7 Hz, 1H), 4.02 (t, J = 7.6 Hz, 1H), |
| | 3.90-3.85 (m, 1H), 3.81 (q, J = 7.6 Hz, 1H), 3.74-3.62 (m, 2H), 3.56 (t, J = 7.7 Hz, 1H), 3.41-3.35 |
| | (m, 2H), 2.81 (t, J = 11.6 Hz, 2H), 2.43-2.29 (m, 1H), 2.18 (s, 2H), 2.07-1.93 (m, 3H), 1.80 (q, J = |
| | 11.9 Hz, 2H), 1.67 (d, J = 13.3 Hz, 1H), 1.50 (t, J = 15.1 Hz, 3H), 1.14 (t, J = 7.3 Hz, 3H), 0.86 |
| | (t, J = 6.6 Hz, 2H). |
| 588 | ¹H NMR (400 MHz, DMSO-d₆) δ 10.67 (s, 1H), 8.20-7.92 (m, 2H), 7.73 (s, 1H), 7.67-7.47 (m, |
| | 3H), 7.13 (t, J = 6.7 Hz, 1H), 5.65-5.40 (m, 2H), 5.32 (s, 1H), 4.01 (t, J = 7.6 Hz, 1H), 4.00-3.90 |
| | (m, 1H), 3.80 (q, J = 7.6 Hz, 1H), 3.69 (s, 2H), 3.56 (t, J = 7.7 Hz, 1H), 3.40-3.34 (m, 2H), 2.80 |
| | (t, J = 11.6 Hz, 2H), 2.33-2.24 (m, 1H), 2.17 (s, 1H), 2.07-1.89 (m, 3H), 1.78 (t, J = 11.6 Hz, 2H), |
| | 1.66 (d, J = 12.7 Hz, 1H), 1.52 (d, J = 12.4 Hz, 2H), 1.23 (s, 3H), 1.13 (t, J = 7.3 Hz, 3H), 0.84 |
| | (d, J = 7.0 Hz, 1H) |
| 589 | ¹H NMR (400 MHz, DMSO-d₆) δ 10.69 (s, 1H), 8.06 (d, J = 8.2 Hz, 1H), 7.94 (s, 1H), 7.69-8.10 |
| | (dd, J = 1.8 Hz, 1H), 7.64-7.30 (dd, J = 2.0 Hz, 3H), 7.15 (t, J = 6.7 Hz, 1H), 5.92 (s, 1H), 5.63- |
| | 5.55 (m, 1H), 5.54-5.46 (m, 1H), 3.70 (s, 2H), 3.29 (s, 3H), 2.81 (t, J = 11.7 Hz, 2H), 2.17 (s, |
| | 2H), 2.05 (d, J = 6.6 Hz, 2H), 1.80 (q, J = 11.5 Hz, 2H), 1.66 (s, 2H), 1.53 (d, J = 12.1 Hz, 2H), |
| | 1.24 (s, 1H), 1.02 (t, J = 3.4 Hz, 2H), 0.85 (t, J = 3.8 Hz, 3H). |
| 590 | ¹H NMR (400 MHz, DMSO-d₆) δ 10.69 (s, 1H), 8.06 (d, J = 5.1 Hz, 2H), 7.63 (d, J = 1.7 Hz, |
| | 3H), 7.14 (d, J = 6.8 Hz, 1H), 6.44 (d, J = 2.4 Hz, 1H), 6.10 (s, 1H), 5.68-5.54 (m, 1H), 5.55-5.43 |
| | (m, 1H), 4.96 (s, 1H), 3.71 (d, J = 8.1 Hz, 5H), 3.53 (d, J = 6.2 Hz, 2H), 2.81 (m, J = 11.6 Hz, |
| | 2H), 2.31 (s, 1H), 2.18 (s, 2H), 2.06 (s, 2H), 1.93-1.72 (m, 2H), 1.53 (d, J = 12.3 Hz, 2H), 1.24 (s, |
| | 2H), 1.14-1.04 (m, 2H), 1.04-0.90 (m, 2H). |
| 618 | ¹H NMR (400 MHz, DMSO) δ 11.05 (s, 1H), 8.55 (s, 1H), 8.24 (s, 1H), 7.85 (d, J = 8.5 Hz, 1H), |
| | 7.66-7.56 (m, 2H), 7.12 (s, 1H), 7.02 (d, J = 7.6 Hz, 1H), 6.46 (s, 1H), 5.58 (s, 1H), 4.12 (s, 2H), |
| | 3.84 (s, 2H), 3.34-3.32(m, 5H) 3.25-3.09 (m, 6H), 2.80 (d, J = 11.8 Hz, 1H), 2.31 (s, 1H), 1.69- |
| | 1.51 (m, 3H), 1.37 (s, 3H), 1.29-1.07 (m, 6H). |
| 619 | ¹H NMR (400 MHz, DMSO-d₆) δ 12.44 (s, 1H), 10.23 (s, 1H), 8.52 (s, 1H), 8.20 (s, 1H), 8.08 (d, |
| | J = 8.6 Hz, 1H), 7.63-7.51 (m, 2H), 7.26 (d, J = 2.2 Hz, 1H), 7.12 (dd, J = 8.7, 2.1 Hz, 1H), 6.30 |
| | (t, J = 6.1 Hz, 1H), 5.45 (dd, J = 15.4, 10.3 Hz, 1H), 4.31 (s, 2H), 4.18 (s, 1H), 3.22-320 (m, 1H), |
| | 3.12 (d, J = 11.1 Hz, 2H), 2.87 (t, J = 12.1 Hz, 2H), 2.54 (s, 6H), 1.67 (d, J = 12.3 Hz, 2H), 1.60 |
| | (d, J = 6.3 Hz, 1H), 1.37 (s, 3H), 1.31 (s, 3H), 1.21 (t, J = 7.3 Hz, 3H), 1.16-1.12 (m, 2H). |
| 621 | ¹H NMR (400 MHz, DMSO-d₆) δ 10.73 (s, 1H), 8.05 (d, J = 8.1 Hz, 1H), 7.94 (s, 1H), 7.65-7.63 |
| | (m, 4H), 7.45 (s, 1H), 7.14 (t, J = 6.7 Hz, 1H), 5.94 (s, 1H), 5.65-5.43 (m, 2H), 3.12 (d, J = 1.5 |
| | Hz, 3H), 2.80 (t, J = 11.6 Hz, 2H), 2.17 (s, 2H), 2.05 (s, 2H), 1.83-1.73 (m, 2H), 1.66 (s, 2H), |
| | 1.53 (d, J = 12.3 Hz, 2H), 1.03 (q, J = 4.7 Hz, 2H), 0.99-0.96 (m, 1H), 0.89-0.82 (m, 2H), 0.81 (d, |
| | J = 1.5 Hz, 6H). |
| 622 | ¹H NMR (400 MHz, DMSO-d₆) δ 10.71 (s, 1H), 8.40 (s, 1H), 8.14 (s, 1H), 8.07 (d, J = 8.1 Hz, |
| | 1H), 7.79-7.55 (m, 3H), 6.91 (t, J = 6.6 Hz, 1H), 5.65-5.46 (m, 2H), 4.26 (s, 2H), 4.06-3.95 (m, |
| | 2H), 3.80 (t, J = 5.2 Hz, 2H), 3.68 (s, 2H), 3.29 (s, 3H), 2.82 (t, J = 11.8 Hz, 2H), 2.31 (s, 1H), |
| | 2.18 (s, 2H), 2.04 (d, J = 17.7 Hz, 2H), 1.86-1.74 (m, 2H), 1.66 (s, 1H), 1.54 (d, J = 12.1 Hz, 2H), |
| | 1.24 (s, 2H) |
| 623 | ¹H NMR (400 MHz, DMSO-d₆) δ 10.72 (s, 1H), 8.40 (s, 1H), 8.14 (s, 1H), 8.05 (d, J = 8.0 Hz, |
| | 1H), 7.65-7.43 (m, 3H), 6.91 (t, J = 6.6 Hz, 1H), 5.65-5.55 (m, 1H), 5.51 (q, J = 7.5 Hz, 1H), 4.94 |
| | (s, 1H), 4.26 (s, 2H), 4.02 (dd, J = 6.1, 4.3 Hz, 2H), 3.80 (t, J = 5.3 Hz, 2H), 3.72 (q, J = 6.0 Hz, |
| | 3H), 3.53 (t, J = 6.2 Hz, 2H), 3.29 (s, 1H), 2.81 (t, J = 11.9 Hz, 2H), 2.31 (s, 1H), 2.17 (s, 2H), |
| | 2.03 (dd, J = 21.7, 7.7 Hz, 2H), 1.86-1.72 (m, 2H), 1.66 (s, 1H), 1.53 (d, J = 11.9 Hz, 2H), 1.24 |
| | (s, 2H). |
| 626 | ¹H NMR (400 MHz, DMSO-d₆) δ 10.92 (s, 1 H), 8.36 (s, 1H), 8.07 (s, 1H), 7.86 (d, J = 8.5 Hz, |
| | 1H), 7.72 (s, 1H), 7.68-7.56 (m, 1H), 7.05 (d, J = 2.3 Hz, 1H), 6.99 (dd, J = 8.7, 2.0 Hz, 1H), |
| | 5.65-5.47 (m, 2H), 5.33 (t, J = 4.7 Hz, 1H), 4.02 (t, J = 7.6 Hz, 1H), 3.90 (m, 1H), 3.80 (q, J = 7.6 |
| | Hz, 2H), 3.67 (s, 2H), 3.56 (t, J = 7.7 Hz, 2H), 3.18 (t, J = 8.1 Hz, 2H), 2.38-2.24 (m, 2H), 2.17 |
| | (s, 2H), 2.09-2.01 (m, 2H), 1.95 (m, 2H), 1.79 (m, 1H), 1.66 (s, 1H), 1.53 (d, J = 12.2 Hz, 1H), |
| | 1.24 (s, 3H), 1.20 (m, 2H), 0.86 (t, J = 6.8 Hz, 2H). |
| 627 | ¹H NMR (400 MHz, DMSO-d₆) δ 10.89 (s, 1H), 8.07 (s, 1H), 7.88 (d, J = 8.8 Hz, 1H), 7.73 (s, |
| | 1H), 7.62 (q, J = 8.4 Hz, 1H), 7.07 (d, J = 6.9 Hz, 1H), 7.01 (d, J = 8.7 Hz, 1H), 5.65-5.46 (m, |
| | 2H), 5.34 (d, J = 4.9 Hz, 1H), 4.02 (t, J = 7.6 Hz, 1H), 3.90 (m, 1H), 3.80 (q, J = 7.5 Hz, 1H), |
| | 3.67 (s, 2H), 3.56 (t, J = 7.7 Hz, 2H), 3.20 (s, 2H), 2.67 (t, J = 11.6 Hz, 2H), 2.35-2.23 (m, 2H), |
| | 2.17 (s, 2H), 2.05 (d, J = 6.7 Hz, 1H), 2.02-1.91 (m, 2H), 1.81 (d, J = 12.5 Hz, 1H), 1.66 (s, 1H), |
| | 1.53 (d, J = 12.3 Hz, 1H), 1.46 (s, 1H), 1.24 (s, 3H), 1.20 (t, J = 7.2 Hz, 2H), 0.86 (t, J = 6.8 Hz, |
| | 2H). |
| 630 | ¹H NMR (400 MHz, DMSO-d₆) δ 12.59 (s, 1H), 10.23 (s, 1H), 8.07 (d, J = 8.6 Hz, 1H), 7.85 (d, J = |
| | 2.7 Hz, 1H), 7.57 (d, J = 8.3 Hz, 1H), 7.53 (d, J = 8.3 Hz, 1H), 7.25 (d, J = 2.2 Hz, 1H), 7.12 |
| | (dd, J = 8.7, 2.1 Hz, 1H), 6.68 (d, J = 6.3 Hz, 1H), 5.60-5.44 (m, 1H), 5.38 (d, J = 11.3, 5.7 Hz, |
| | 1H), 4.34 (s, 2H), 4.17 (d, J = 4.7 Hz, 2H), 3.21 (d, J = 7.3 Hz, 2H), 3.11 (d, J = 11.3 Hz, 2H), |
| | 2.92-2.77 (m, 4H), 2.51 (s, 2H), 2.49 (s, 2H), 2.19 (s, 3H), 1.97 (d, J = 11.5 Hz, 2H), 1.86 (d, J = |
| | 12.7 Hz, 2H), 1.73-1.58 (m, 3H), 1.31 (s, 3H), 1.21 (d, J = 7.3 Hz, 3H). |
| 631 | ¹H NMR (400 MHz, DMSO) δ 10.61 (s, 1H), 8.02 (d, J = 8.1 Hz, 1H), 7.74 (d, J = 8.3 Hz, 1H), |
| | 7.67-7.60 (m, 2H), 7.51 (d, J = 8.3 Hz, 1H), 6.43 (t, J = 6.5 Hz, 1H), 5.57 (m, 1H), 5.51-5.41 (m, |
| | 1H), 4.92 (t, J = 5.4 Hz, 1H), 3.71 (q, J = 6.0 Hz, 2H), 3.64 (s, 2H), 3.52 (t, J = 6.3 Hz, 2H), 3.25 |
| | (d, J = 11.3 Hz, 2H), 2.79 (t, J = 11.6 Hz, 2H), 2.23 (t, J = 6.5 Hz, 2H), 2.10-2.00 (m, 2H), 1.72 |
| | (m, 3H), 1.51 (d, J = 12.1 Hz, 2H). |
| 654 | ¹H NMR (400 MHz, DMS-d₆) δ 10.86 (s, 1H), 7.98 (d, J = 8.1 Hz, 1H), 7.82-7.73 (m, 1H), 7.70 |
| | (d, J = 2.7 Hz, 1H), 7.64 (dd, J = 8.1, 1.7 Hz, 1H), 7.50-7.37 (m, 1H), 6.81 (d, J = 5.6 Hz, 1H), |
| | 5.95 (d, J = 16.5 Hz, 1H), 5.49 (d, J = 15.3 Hz, 1H), 5.00-4.76 (m, 1H), 4.31-3.96 (m, 2H), 3.99- |
| | 3.78 (m, 1H), 3.75-3.61 (m, 2H), 3.52 (d, J = 6.3 Hz, 1H), 3.17 (d, J = 5.3 Hz, 1H), 3.09-2.82 (m, |
| | 2H), 2.51-2.44 (m, 5H), 2.11 (d, J = 27.8 Hz, 2H), 1.62 (d, J = 12.9 Hz, 2H), 1.21 (s, 1H). |
| 655 | ¹H NMR (400 MHz, DMSO-d₆) δ 12.66 (d, J = 3.5 Hz, 1H), 8.31 (d, J = 8.2, 2.3 Hz, 1H), 7.99 (q, |
| | J = 1.9 Hz, 1H), 7.80 (d, J = 10.8, 5.0, 3.6 Hz, 1H), 7.49-7.39 (m, 1H), 6.64 (d, J = 8.0 Hz, 1H), |
| | 5.46 (d, J = 22.0 Hz, 2H), 4.94 (d, J = 5.4, 1.6 Hz, 1H), 4.50-4.21 (m, 2H), 4.17 (s, 2H), 3.73 (dd, |
| | J = 7.0, 5.2 Hz, 2H), 3.56 (d, J = 6.1 Hz, 2H), 3.11 (dd, J = 23.8, 8.6 Hz, 4H), 2.49 (s, 3H), 1.67 |
| | (d, J = 12.8 Hz, 2H), 1.40-1.27 (m, 3H). |
| 656 | ¹H NMR (400 MHz, DMSO-d₆) δ 10.68 (s, 1H), 8.11 (d, J = 2.4 Hz, 1H), 8.06 (d, J = 8.1 Hz, |
| | 1H), 7.66-7.62 (m, 2H), 7.61-7.58 (m, 2H), 6.74 (t, J = 6.7 Hz, 1H), 6.49 (d, J = 2.5 Hz, 1H), |
| | 5.60-5.57 (m, 1H), 5.55-5.45 (m, 1H), 4.11 (s, 2H), 3.67 (s, 2H), 3.28 (d, J = 12.1 Hz, 4H), 2.81 |
| | (t, J = 11.6 Hz, 2H), 2.17 (s, 2H), 2.09-2.02 (m, 2H), 1.77 (t, J = 3.7 Hz, 2H), 1.69-1.63 (m, 1H), |
| | 1.63-1.48 (m, 4H), 1.14 (t, J = 7.3 Hz, 3H). |
| 657 | ¹H NMR (400 MHz, DMSO-d₆) δ 10.67 (s, 1H), 8.06 (d, J = 8.1 Hz, 1H), 7.99 (s, 1H), 7.69-7.59 |
| | (m, 3H), 7.59 (d, J = 1.7 Hz, 1H), 7.23 (t, J = 6.7 Hz, 1H), 5.60-5.57 (m, 1H), 5.55-5.44 (m, 1H), |
| | 3.96 (s, 4H), 3.70 (s, 2H), 3.28 (d, J = 11.1 Hz, 1H), 2.81 (t, J = 11.7 Hz, 2H), 2.27 (s, 3H), 2.18 |
| | (s, 3H), 2.08-2.02 (m, 2H), 1.79-1.76 (m, 2H), 1.65 (s, 1H), 1.52 (d, J = 12.1 Hz, 2H), 1.14 (t, J = |
| | 7.3 Hz, 3H), 0.91-0.84 (m, 2H), 0.84-0.77 (m, 2H). |
| 658 | ¹H NMR (400 MHz, DMSO-d₆) δ 11.01 (s, 1H), 7.91 (s, 1H), 7.83 (dd, J = 8.5, 2.6 Hz, 1H), 7.67- |
| | 7.60 (m, 2H), 7.57 (d, J = 8.3 Hz, 1H), 7.04 (t, J = 6.8 Hz, 1H), 6.94 (s, 1H), 5.91 (s, 1H), 5.55- |
| | 5.53 (m, 2H), 3.97 (s, 1H), 3.77-3.44 (m, 1H), 3.17 (d, J = 11.1 Hz, 2H), 2.95 (d, J = 7.7 Hz, 2H), |
| | 2.66 (t, J = 11.4 Hz, 3H), 2.36-2.28 (m, 1H), 2.17 (s, 2H), 2.07 (d, J = 7.3 Hz, 1H), 1.80 (t, J = |
| | 11.8 Hz, 2H), 1.65 (s, 1H), 1.52 (d, J = 12.4 Hz, 2H), 1.22 (d, J = 16.9 Hz, 2H), 1.06-0.98 (m, |
| | 2H), 0.88-0.81 (m, 2H). |
| 659 | ¹H NMR (400 MHz, DMSO-d₆) δ 10.72 (s, 1H), 8.03 (d, J = 8.4 Hz, 1H), 7.94 (s, 1H), 7.71 (t, J = |
| | 5.7 Hz, 1H), 7.63 (d, J = 2.0 Hz, 3H), 7.55 (d, J = 7.0 Hz, 2H), 7.14 (t, J = 6.8 Hz, 1H), 5.92 (s, |
| | 1H), 5.66-5.44 (m, 2H), 3.69 (s, 2H), 3.26 (d, J = 11.2 Hz, 2H), 2.87-2.72 (m, 4H), 2.17 (s, 2H), |
| | 2.09-2.01 (m, 2H), 1.80 (q, J = 12.3 Hz, 2H), 1.67 (d, J = 12.7 Hz, 1H), 1.52 (d, J = 12.2 Hz, 2H), |
| | 1.06-0.95 (m, 5H), 0.85 (q, J = 4.7 Hz, 2H). |
| 660 | ¹H NMR (400 MHz, DMSO-d₆) δ 12.54 (s, 1H), 8.34 (d, J = 8.2 Hz, 1H), 7.94 (s, 1H), 7.80 (d, J = |
| | 1.8 Hz, 1H), 7.78-7.60 (m, 4H), 7.39-7.31 (m, 2H), 5.92 (s, 1H), 5.50 (t, J = 5.4 Hz, 2H), 3.47 |
| | (dd, J = 10.8, 6.0 Hz, 2H), 3.16 (d, J = 11.1 Hz, 2H), 2.96-2.77 (m, 4H), 2.33-2.25 (m, 2H), 2.18 |
| | (t, J = 7.0 Hz, 2H), 1.94 (q, J = 11.9 Hz, 2H), 1.82 (d, J = 13.8 Hz, 2H), 1.75 (s, 1H), 1.06-0.94 |
| | (m, 5H), 0.89-0.81 (m, 2H). |
| 661 | ¹H NMR (400 MHz, DMSO-d6) δ 12.67 (s, 1H), 8.34 (d, J = 8.2 Hz, 1H), 8.20 (s, 1H), 7.97 (s, |
| | 1H), 7.81 (d, J = 11.8 Hz, 2H), 7.66 (d, J = 8.3 Hz, 1H), 7.57 (d, J = 8.2 Hz, 1H), 7.06 (t, J = 6.4 |
| | Hz, 1H), 5.50 (d, J = 10.8 Hz, 1H), 5.41-5.34 (m, 1H), 4.39-4.33 (m, 2H), 4.20-4.15 (m, 4H), |
| | 3.40 (q, J = 7.4 Hz, 2H), 3.17 (d, J = 11.2 Hz, 2H), 3.08 (t, J = 12.1 Hz, 2H), 1.74-1.65 (m, 4H), |
| | 1.45 (q, J = 4.8 Hz, 2H), 1.35 (s, 3H), 1.14 (t, J = 7.3 Hz, 3H). |
| 662 | ¹H NMR (400 MHz, DMSO-d6) δ 11.14 (s, 1H), 10.17 (s, 1H), 8.21 (s, 1H), 7.86 (d, J = 8.6 Hz, |
| | 1H), 7.81 (s, 1H), 7.65 (d, J = 8.3 Hz, 1H), 7.60 (d, J = 8.3 Hz, 1H), 7.13 (d, J = 2.1 Hz, 1H), 7.10 |
| | (t, J = 5.3 Hz, 1H), 7.03 (dd, J = 8.5, 2.0 Hz, 1H), 5.97 (d, J = 15.6 Hz, 1H), 5.53 (d, J = 15.6 Hz, |
| | 1H), 4.12 (s, 2H), 3.87 (s, 2H), 3.26-3.10 (m, 4H), 2.79 (t, J = 11.8 Hz, 2H), 2.26 (s, 2H), 1.73- |
| | 1.61 (m, 4H), 1.45 (q, J = 4.9 Hz, 2H), 1.23-1.17 (m, 6H) |
| 663 | ¹H NMR (400 MHz, DMSO-d6) δ 10.69 (s, 1H), 8.14 (s, 1H), 8.06 (d, J = 8.1 Hz, 1H), 7.77 (s, |
| | 1H), 7.64 (dd, J = 7.2, 1.5 Hz, 2H), 7.59 (d, J = 1.8 Hz, 1H), 7.09 (s, 1H), 5.89 (s, 1H), 5.58 (s, |
| | 1H), 5.52 (s, 1H), 3.70 (s, 2H), 3.52 (s, 2H), 3.37 (d, J = 7.4 Hz, 2H), 2.81 (t, J = 11.5 Hz, 2H), |
| | 2.31 (s, 1H), 2.18 (s, 2H), 1.80 (d, J = 12.6 Hz, 2H), 1.52 (d, J = 11.8 Hz, 3H), 1.16-1.11 (m, |
| | 4H), 1.04 (s, 2H), 0.89-0.80 (m, 3H). |
| 664 | ¹H NMR (400 MHz, DMSO-d6) δ 10.92 (s, 1H), 10.17 (s, 1H), 8.37 (s, 1H), 8.12 (s, 1H), 7.89 |
| | (dd, J = 8.4, 1.3 Hz, 1H), 7.69-7.56 (m, 2H), 7.06 (s, 1H), 6.83 (t, J = 6.7 Hz, 1H), 6.98-5.55 (m, |
| | 2H), 4.25 (d, J = 1.2 Hz, 2H), 4.01 (dd, J = 6.3, 4.1 Hz, 2H), 3.83-3.75 (m, 2H), 3.66 (s, 2H), |
| | 3.19 (d, J = 10.8 Hz, 2H), 3.09 (d, J = 1.5 Hz, 3H), 2.68 (t, J = 11.7 Hz, 2H), 2.16 (s, 2H), 2.10- |
| | 2.04 (m, 3H), 1.84-1.74 (m, 2H), 1.66 (s, 1H), 1.53 (d, J = 12.3 Hz, 2H). |
| 665 | ¹H NMR (400 MHz, DMSO-d6) δ 10.71 (s, 1H), 8.04 (dd, J = 8.6, 3.6 Hz, 1H), 7.94 (d, J = 3.6 |
| | Hz, 1H), 7.63 (t, J = 2.7 Hz, 4H), 7.58-7.51 (m, 2H), 7.14 (s, 1H), 5.93 (d, J = 3.0 Hz, 1H), 5.69- |
| | 5.42 (m, 1H), 3.69 (s, 2H), 2.78 (t, J = 11.7 Hz, 2H), 2.52 (d, J = 1.6 Hz, 1H), 2.45 (d, J = 4.1 Hz, |
| | 4H), 2.17 (s, 2H), 2.05 (s, 2H), 1.79 (t, J = 11.8 Hz, 2H), 1.66 (s, 1H), 1.53 (d, J = 12.5 Hz, 2H), |
| | 1.24 (s, 1H), 1.03 (dd, J = 7.1, 4.3 Hz, 2H), 0.85 (t, J = 3.7 Hz, 2H). |
| 666 | ¹H NMR (400 MHz, DMSO-d6) δ 12.53 (s, 1H), 8.34 (d, J = 8.3 Hz, 1H), 7.94 (s, 1H), 7.77 (d, J = |
| | 1.8 Hz, 1H), 7.73-7.68 (m, 1H), 7.66 (d, J = 8.2 Hz, 1H), 7.63 (d, J = 5.1 Hz, 2H), 7.33 (d, J = |
| | 8.3 Hz, 2H), 5.94 (s, 1H), 5.50 (d, J = 7.6 Hz, 3H), 4.37 (s, 1H), 3.17 (d, J = 10.7 Hz, 2H), 2.91 (t, |
| | J = 11.3 Hz, 2H), 2.45 (s, 3H), 2.30 (d, J = 21.9 Hz, 2H), 2.18 (s, 2H), 2.02-1.86 (m, 1H), 1.87- |
| | 1.66 (m, 3H), 1.23 (s, 1H), 1.03 (t, J = 3.8 Hz, 2H), 0.89-0.81 (m, 2H). |
| 672 | ¹H NMR (400 MHz, DMSO-d6) δ 10.74 (s, 1H), 8.39 (s, 1H), 8.14 (s, 1H), 8.04 (dd, J = 8.5, 1.2 |
| | Hz, 1H), 7.70-7.60 (m, 2H), 7.58-7.51 (m, 3H), 6.89 (t, J = 6.5 Hz, 1H), 5.66-5.45 (m, 2H), |
| | 4.26 (s, 2H), 4.05-3.98 (m, 2H), 3.80 (t, J = 5.1 Hz, 2H), 3.68 (s, 2H), 3.27 (d, J = 11.4 Hz, 2H), |
| | 2.78 (t, J = 11.6 Hz, 2H), 2.45 (s, 3H), 2.18 (s, 2H), 2.10-2.03 (m, 2H), 1.81 (q, J = 11.8 Hz, |
| | 2H), 1.68 (d, J = 12.6 Hz, 1H), 1.54 (d, J = 12.3 Hz, 2H). |
| 673 | ¹H NMR (400 MHz, DMSO-d6) δ 12.57 (s, 1H), 8.40 (s, 1H), 8.35 (d, J = 8.2 Hz, 1H), 8.13 (s, |
| | 1H), 7.78 (d, J = 1.8 Hz, 1H), 7.74-7.60 (m, 3H), 7.37 (d, J = 8.2 Hz, 1H), 7.14 (t, J = 6.5 Hz, |
| | 1H), 5.54-5.44 (m, 2H), 4.25 (s, 2H), 4.02 (t, J = 5.1 Hz, 2H), 3.80 (t, J = 5.3 Hz, 2H), 3.52- |
| | 3.41 (m, 2H), 3.18 (d, J = 10.9 Hz, 2H), 2.91 (t, J = 11.3 Hz, 2H), 2.46 (d, J = 4.4 Hz, 3H), 2.29 |
| | (d, J = 14.9 Hz, 2H), 2.19 (t, J = 7.0 Hz, 2H), 2.00-1.87 (m, 2H), 1.82 (d, J = 13.5 Hz, 2H), 1.75 |
| | (s, 1H). |

Provided in some embodiments herein is a compound having one of the following structures, a stereoisomer thereof, or a pharmaceutically acceptable salt, solvate, or isotopologue of the compound or the stereoisomer:

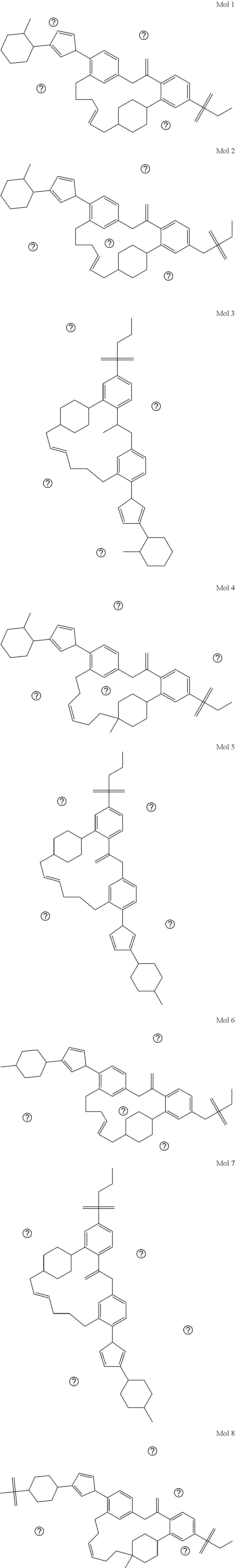

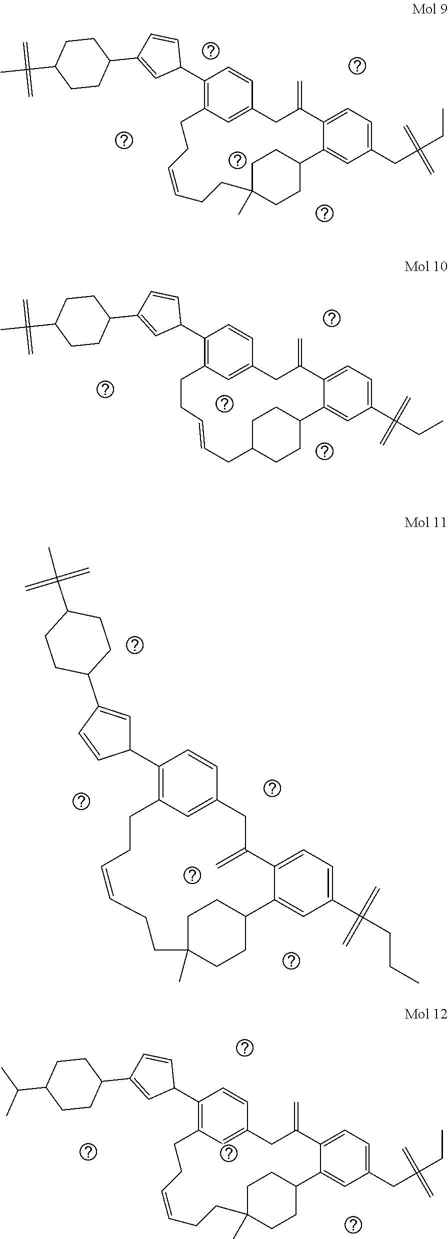

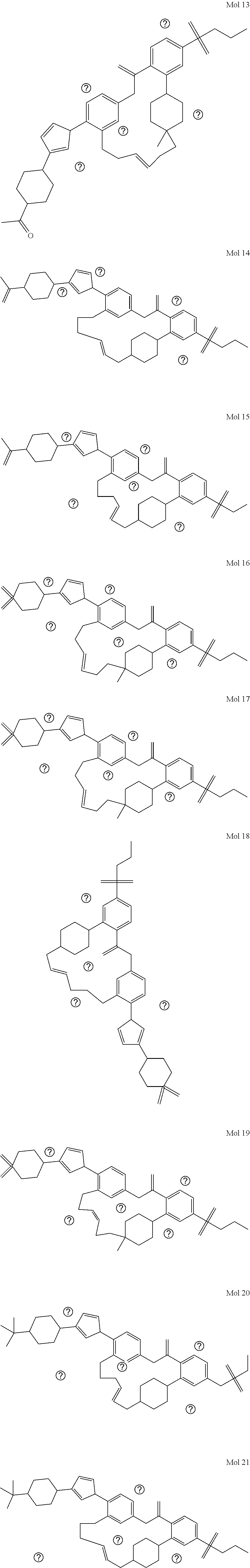

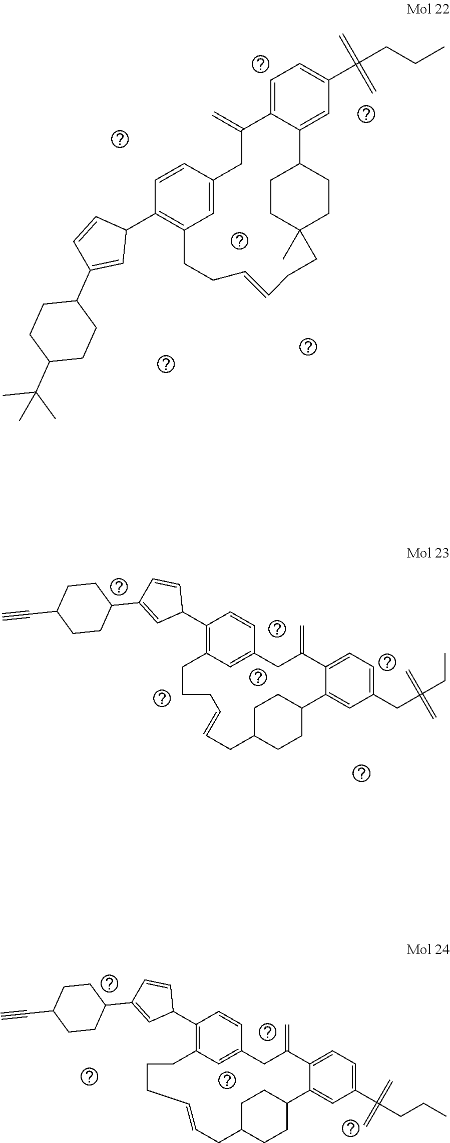

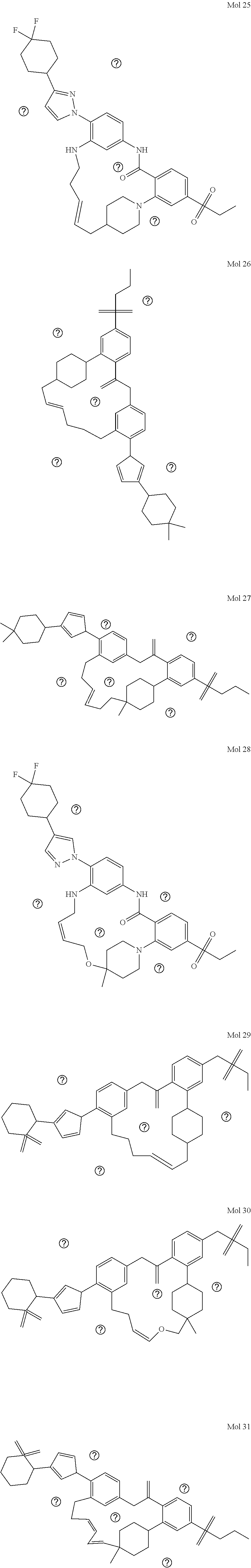

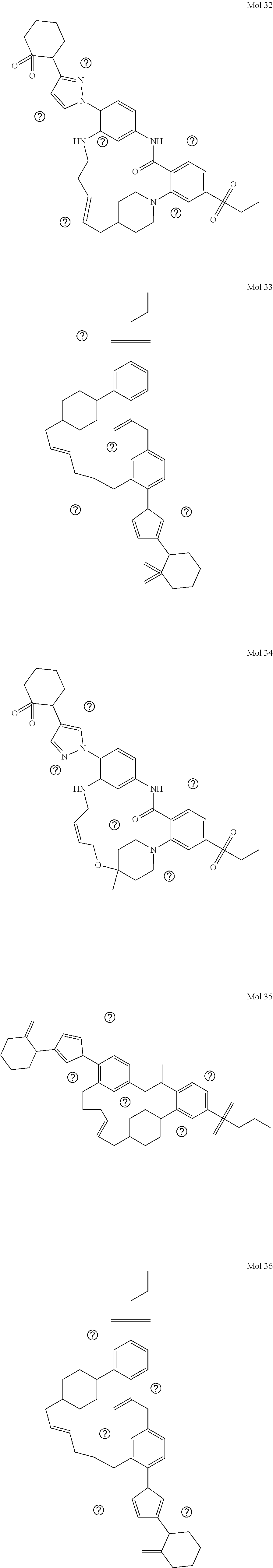

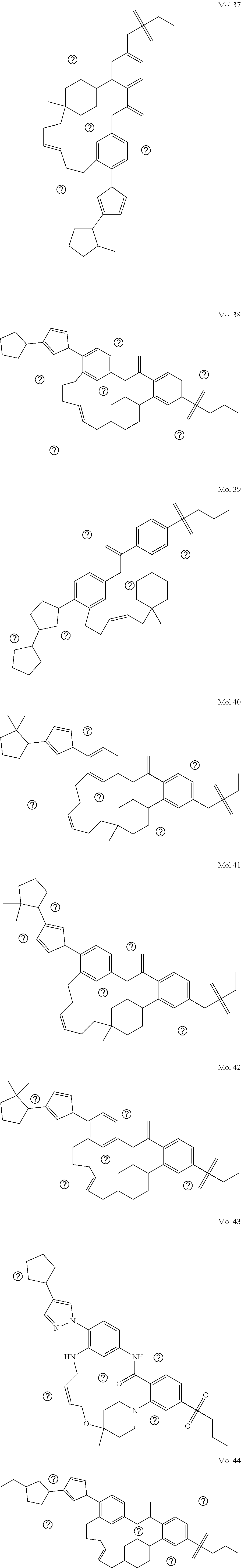

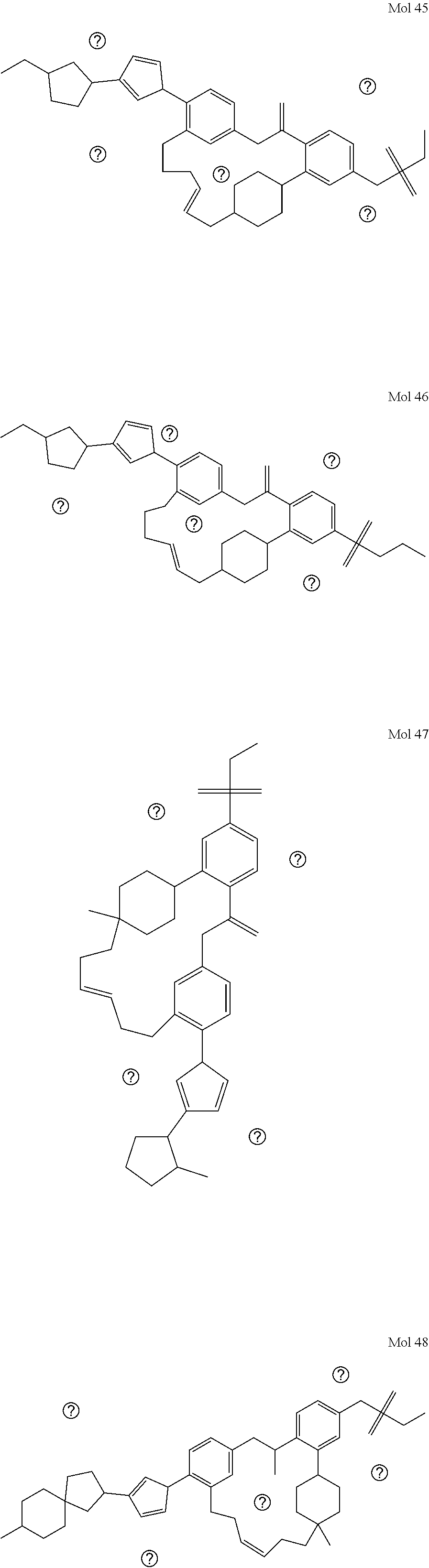

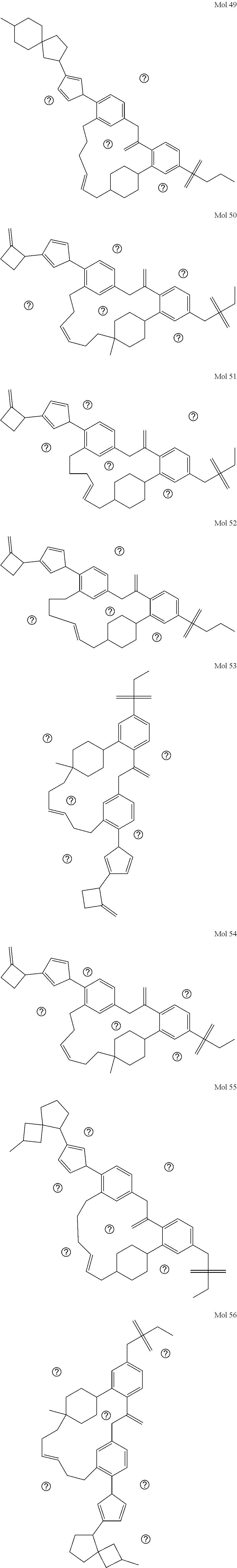

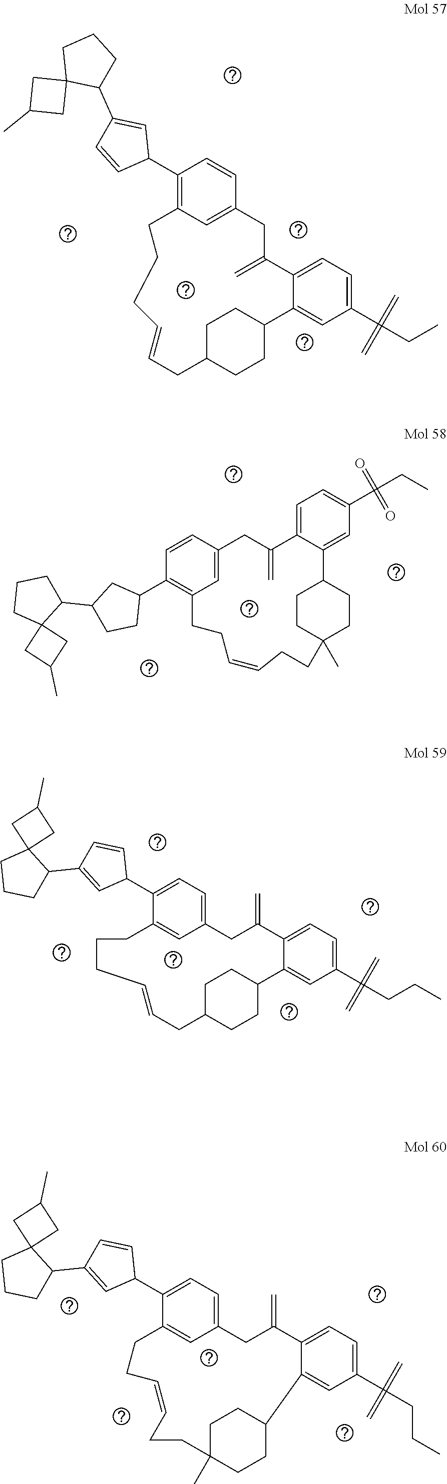

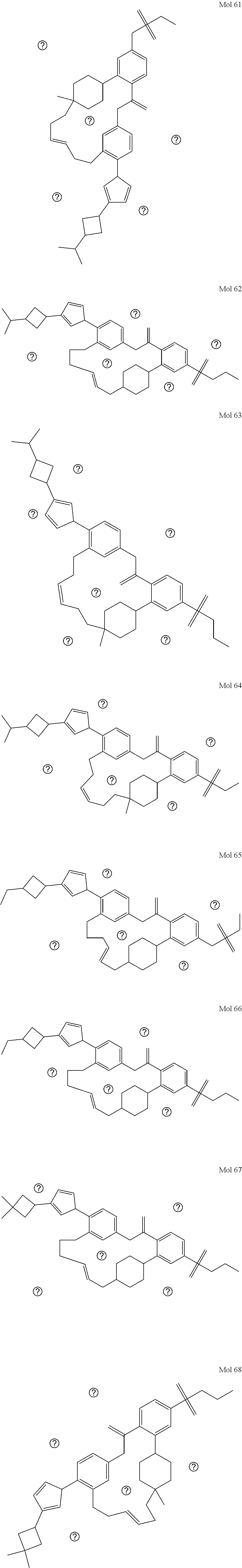

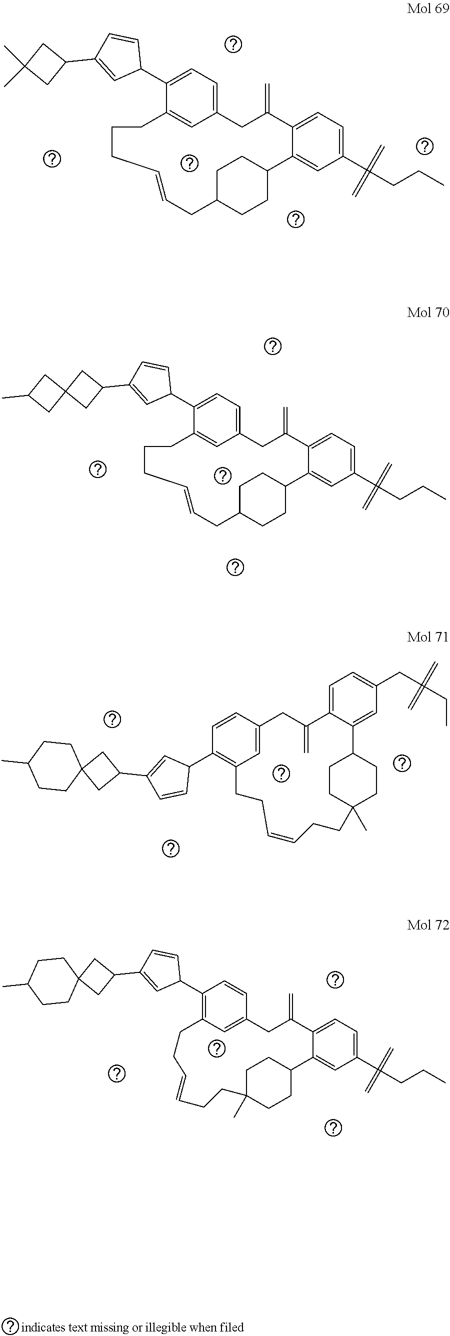

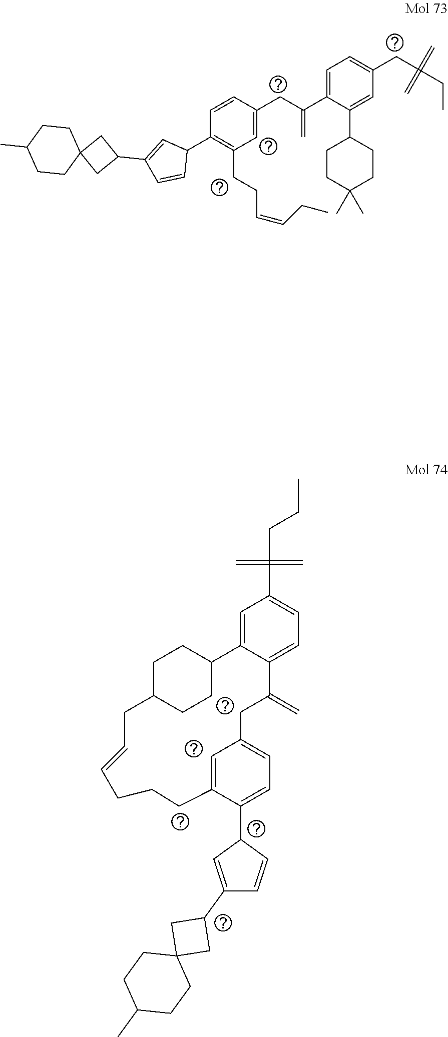

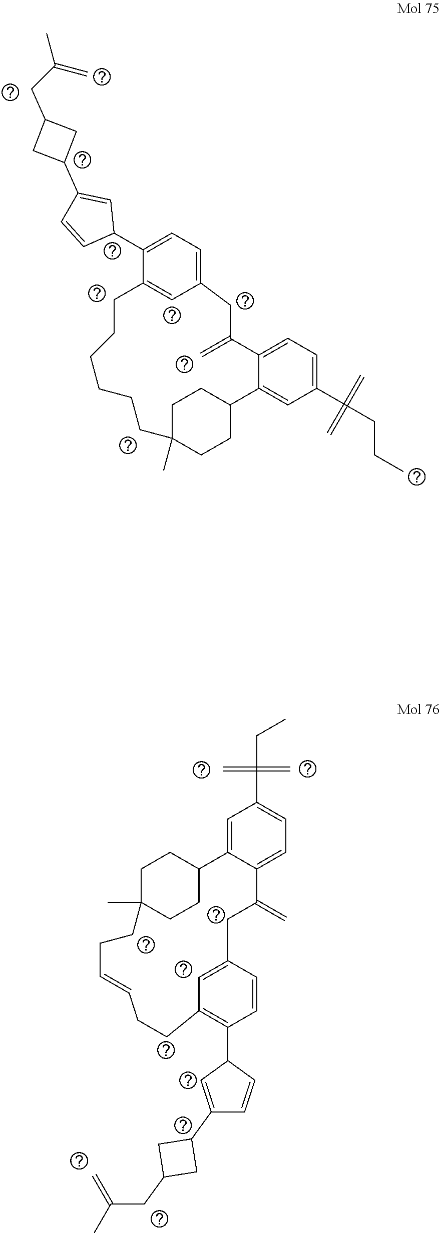

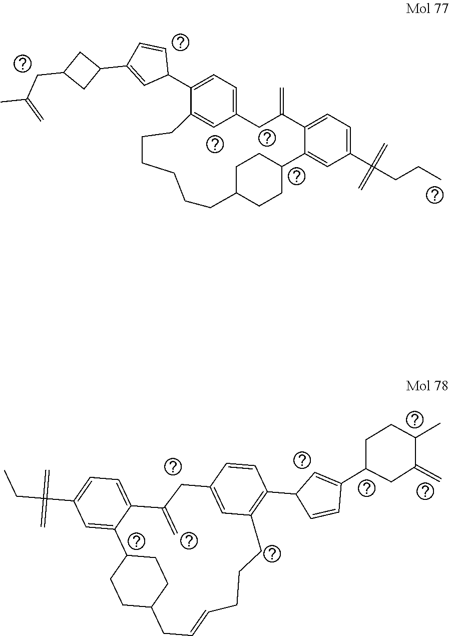

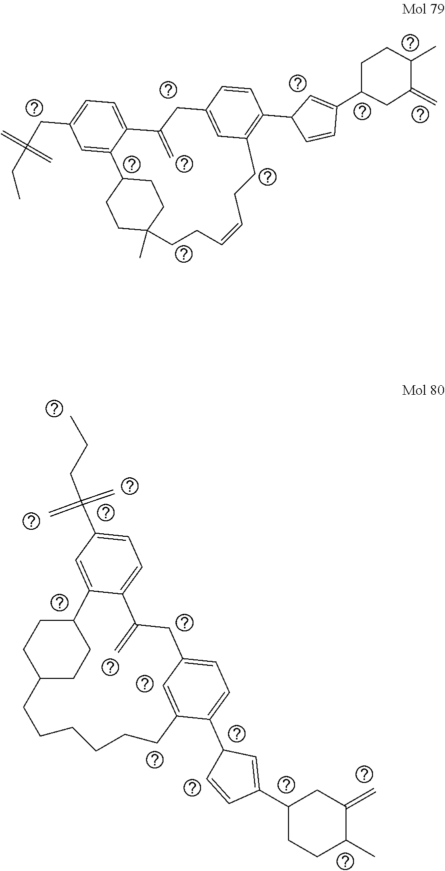

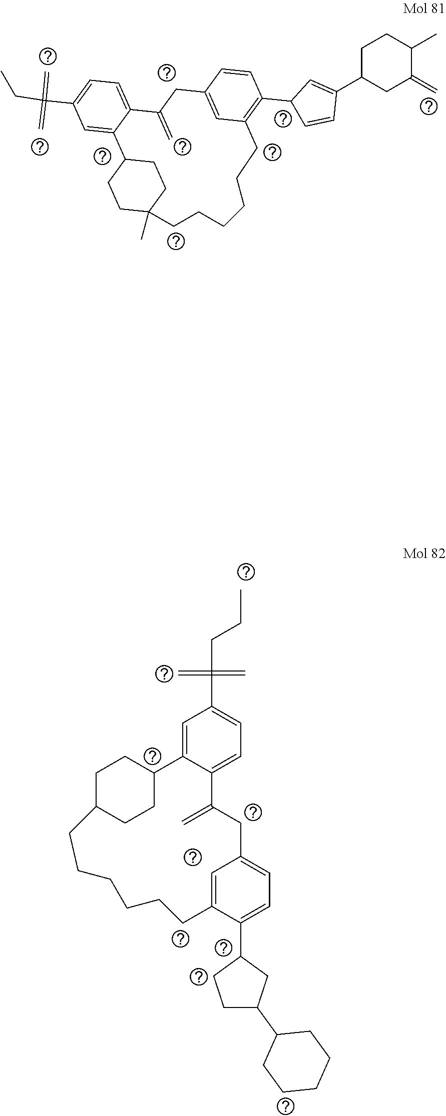

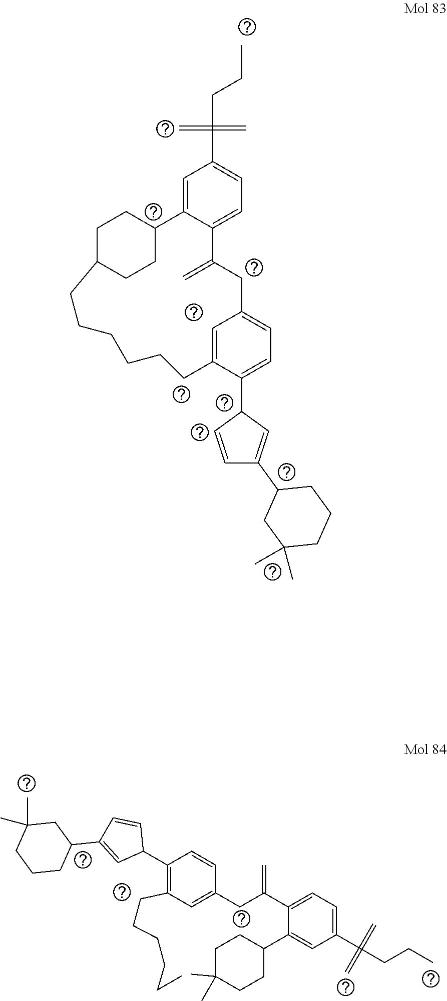

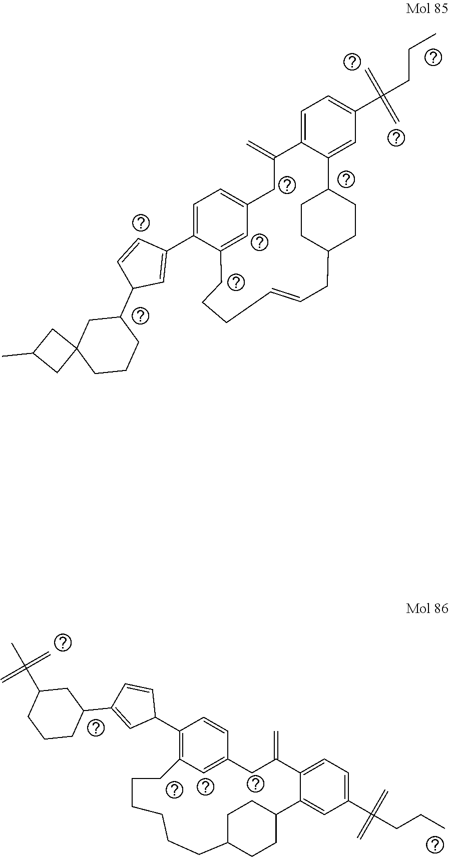

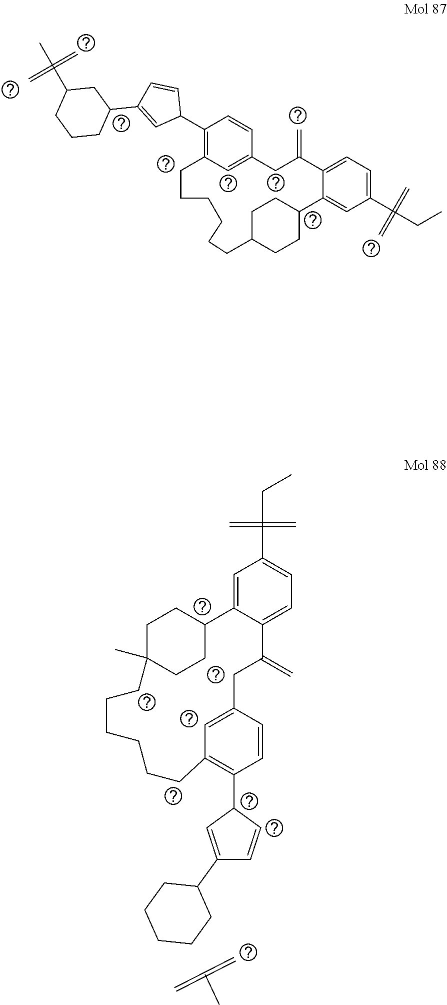

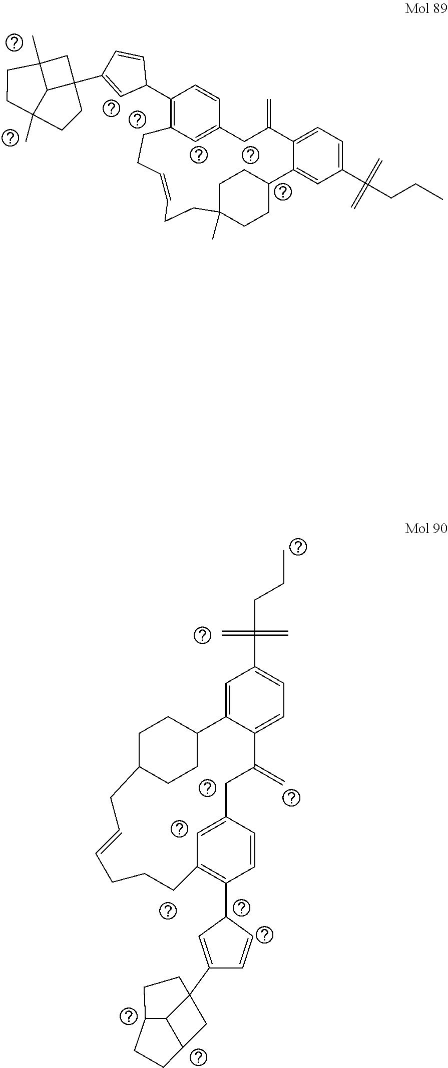

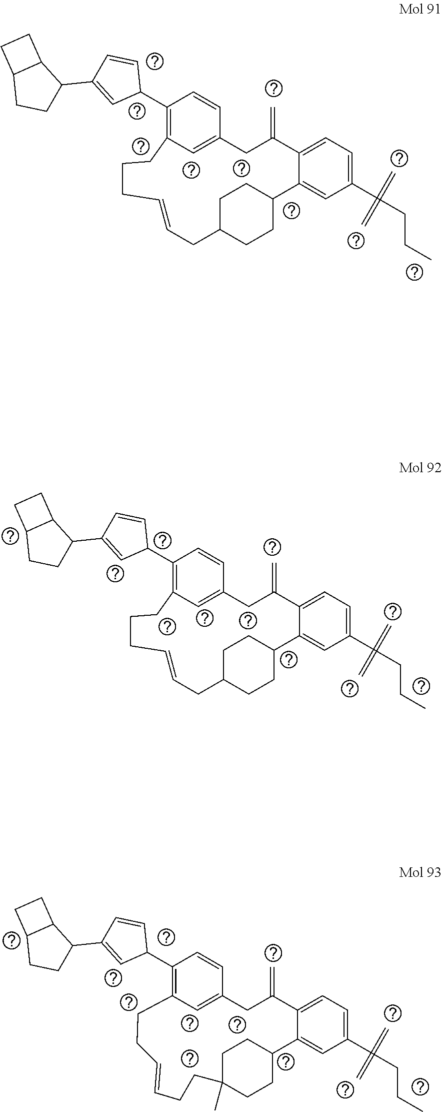

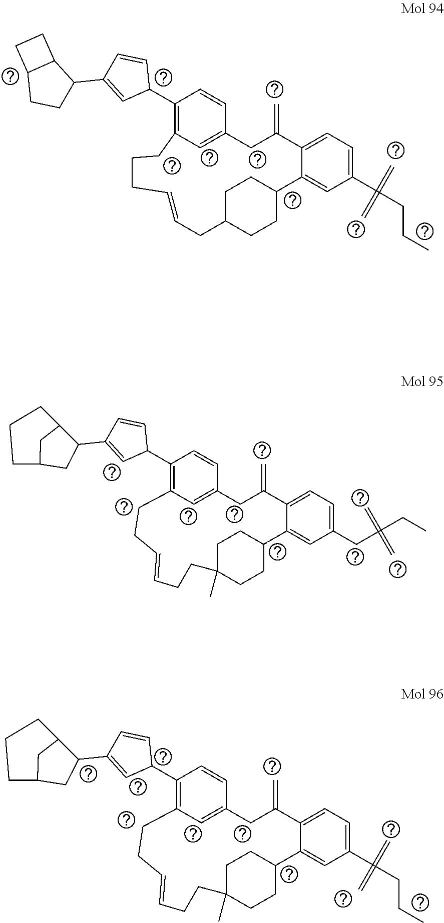

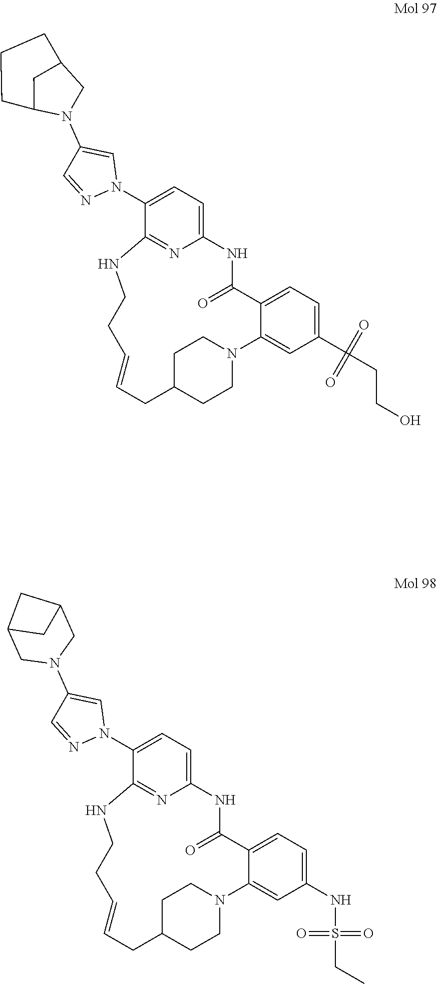

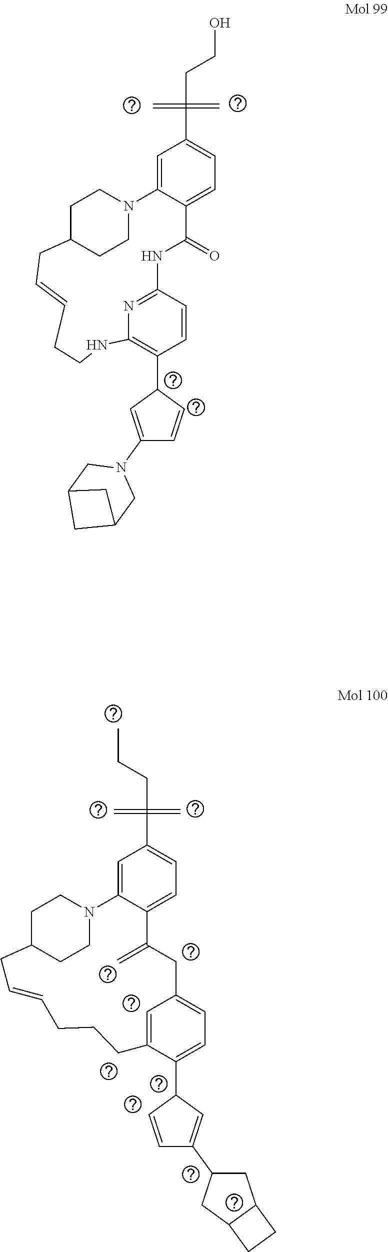

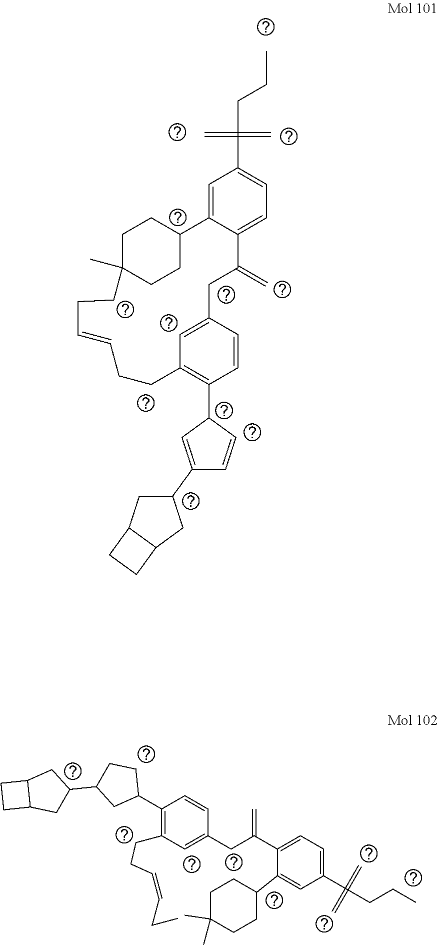

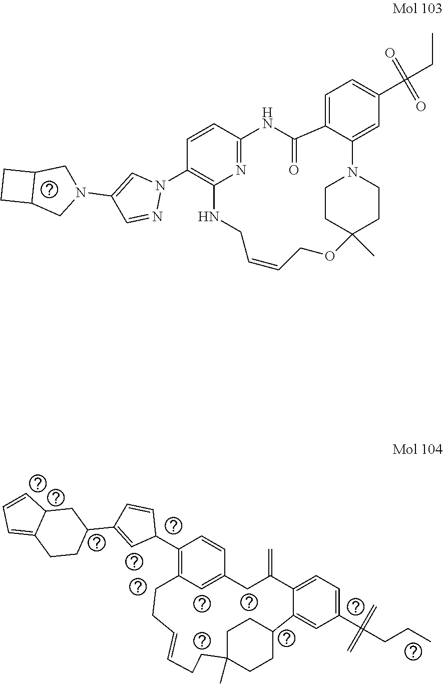

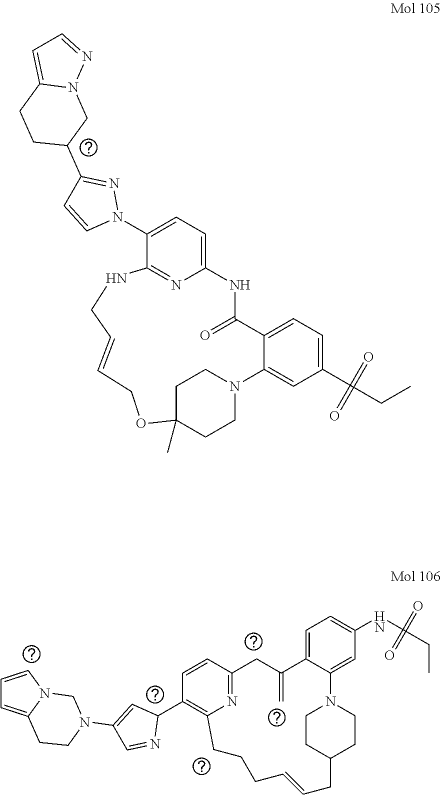

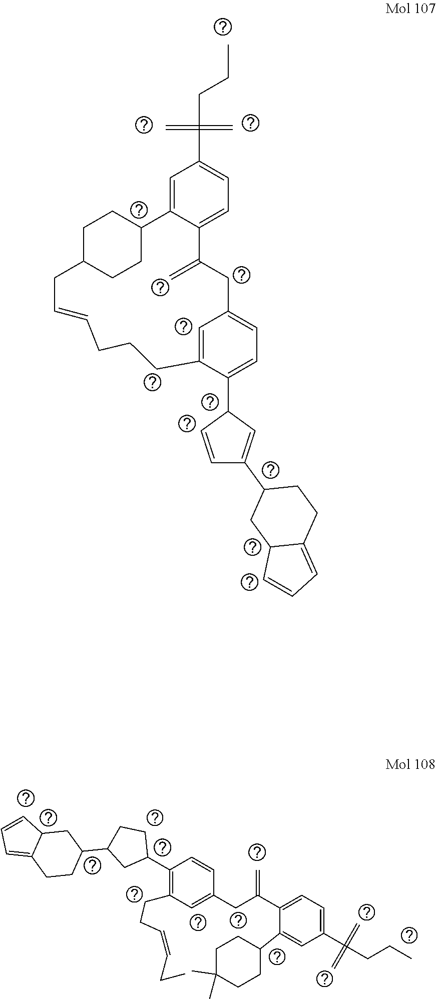

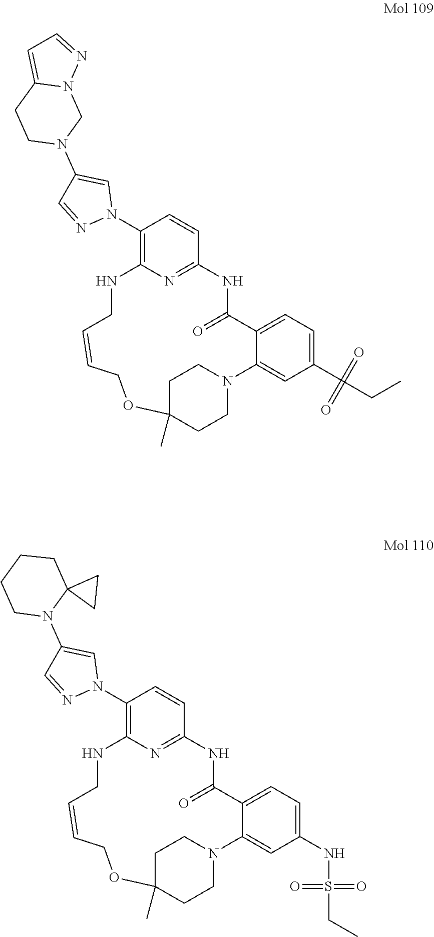

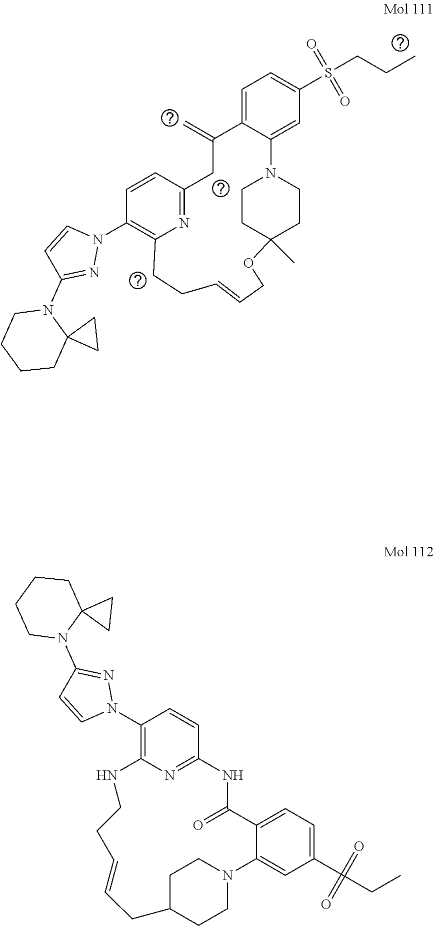

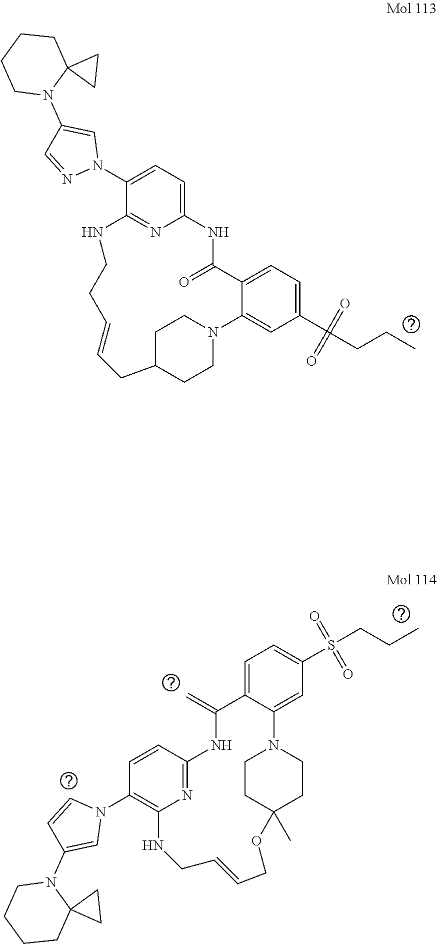

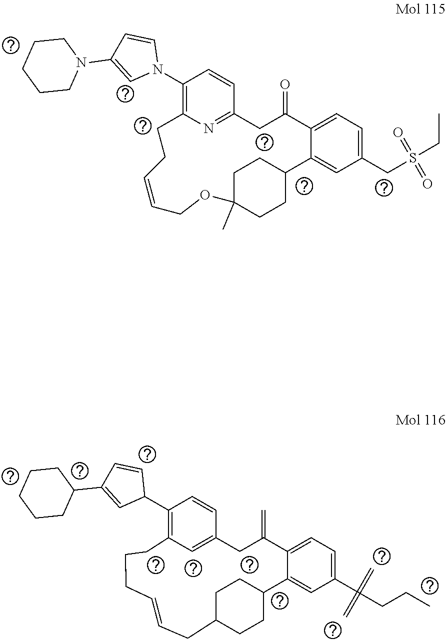

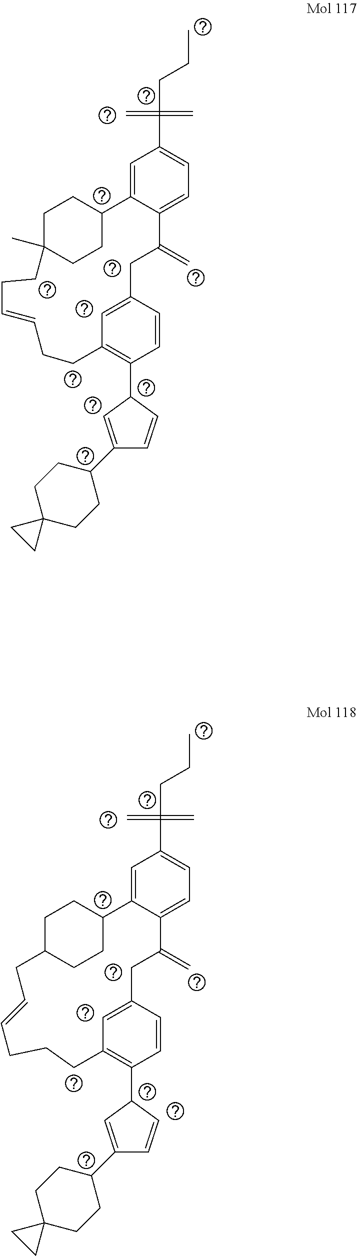

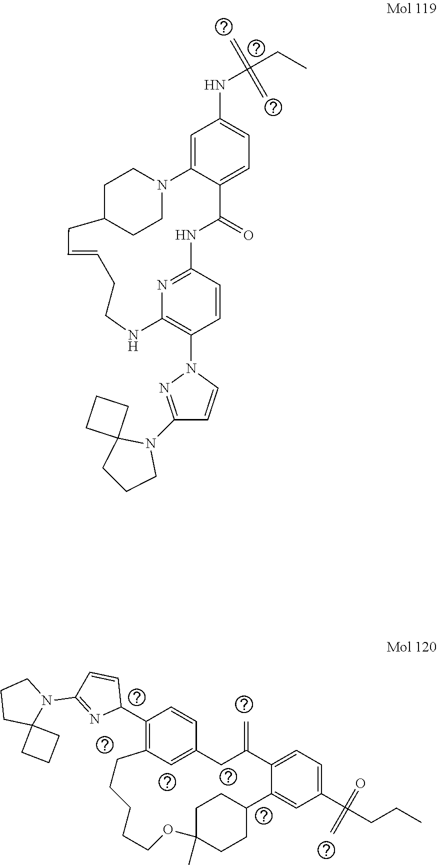

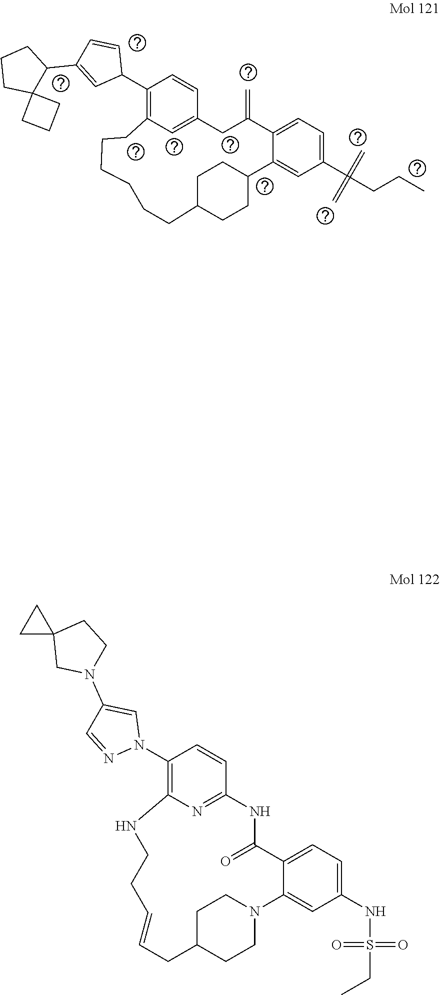

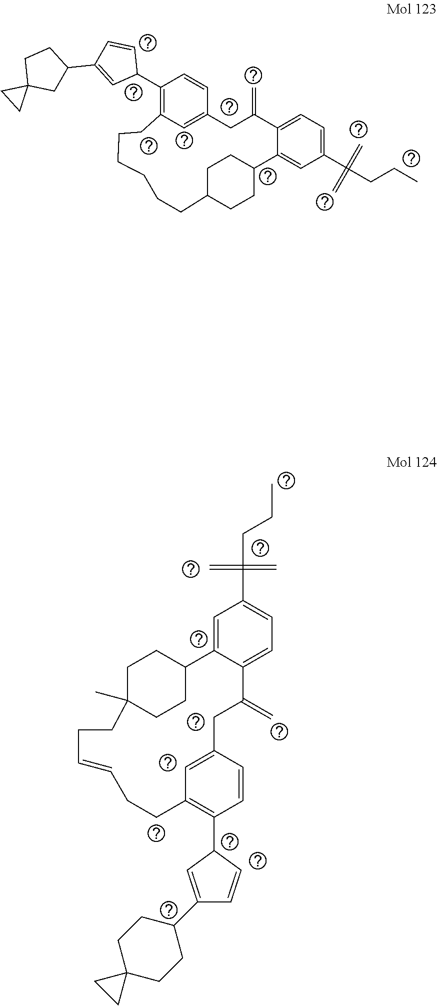

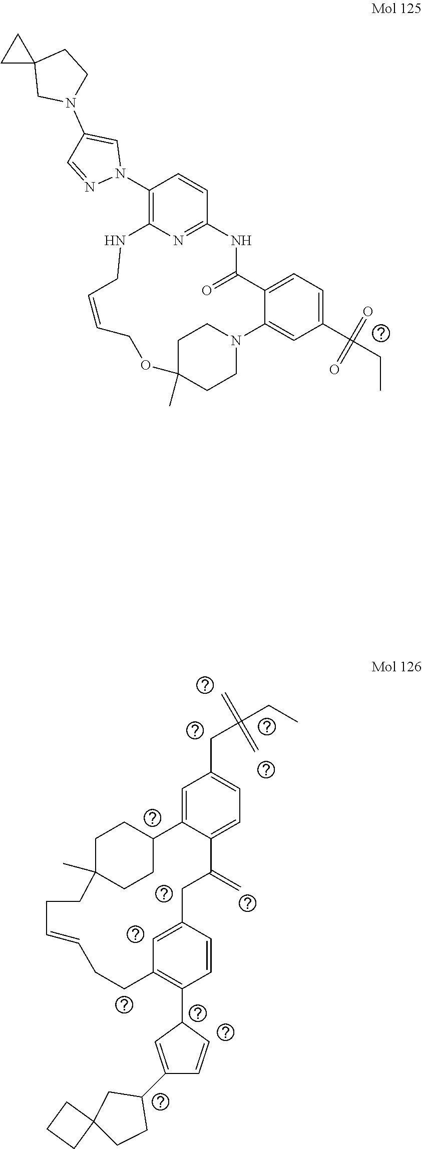

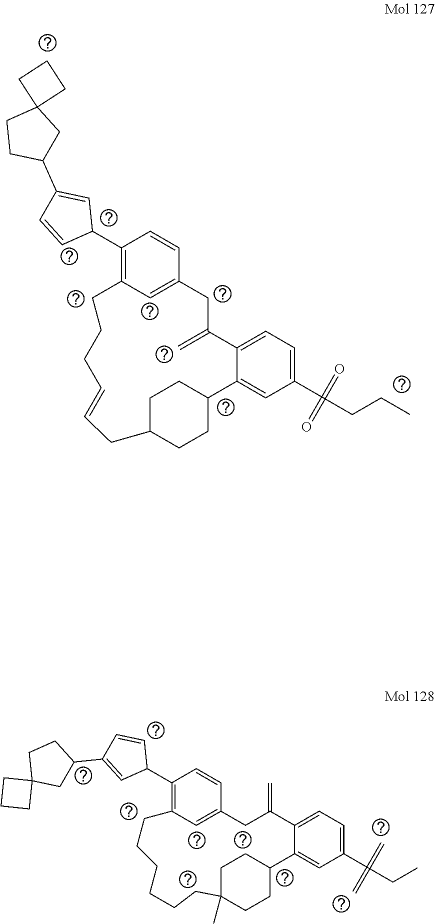

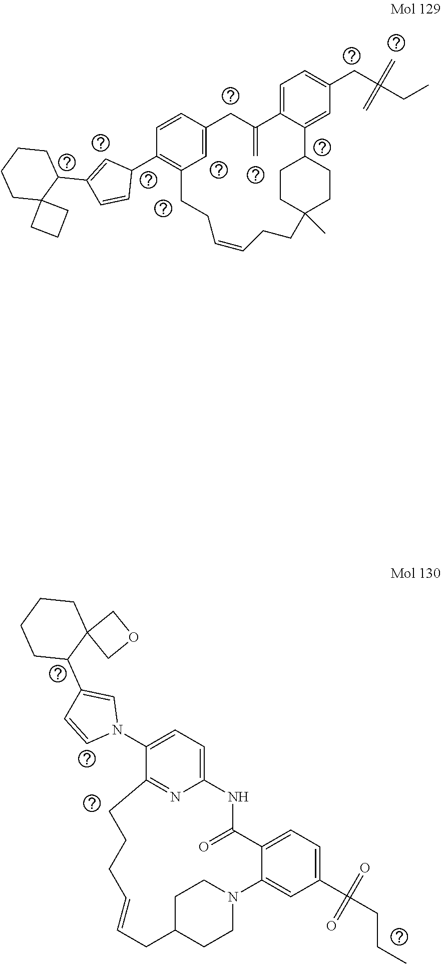

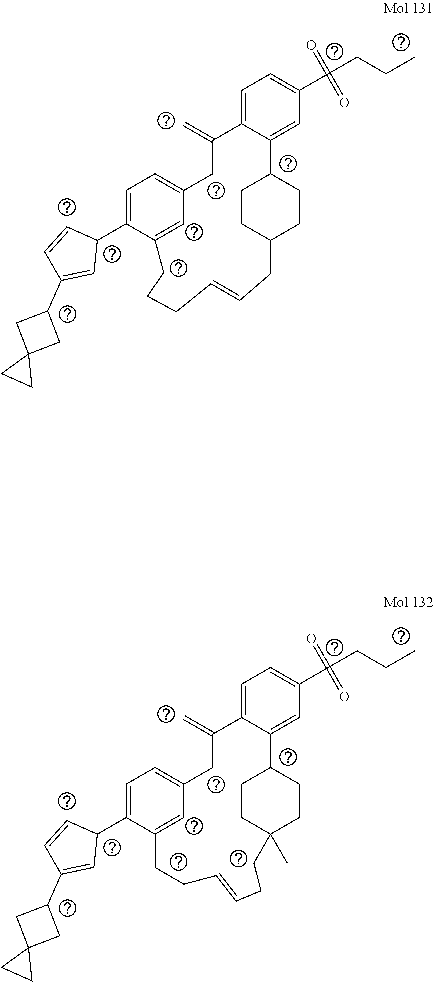

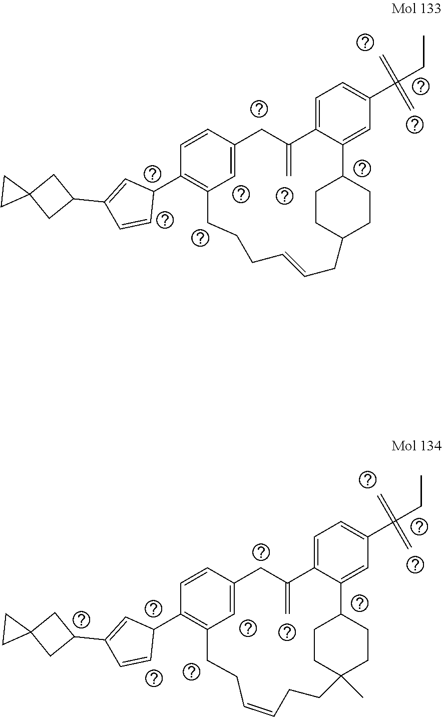

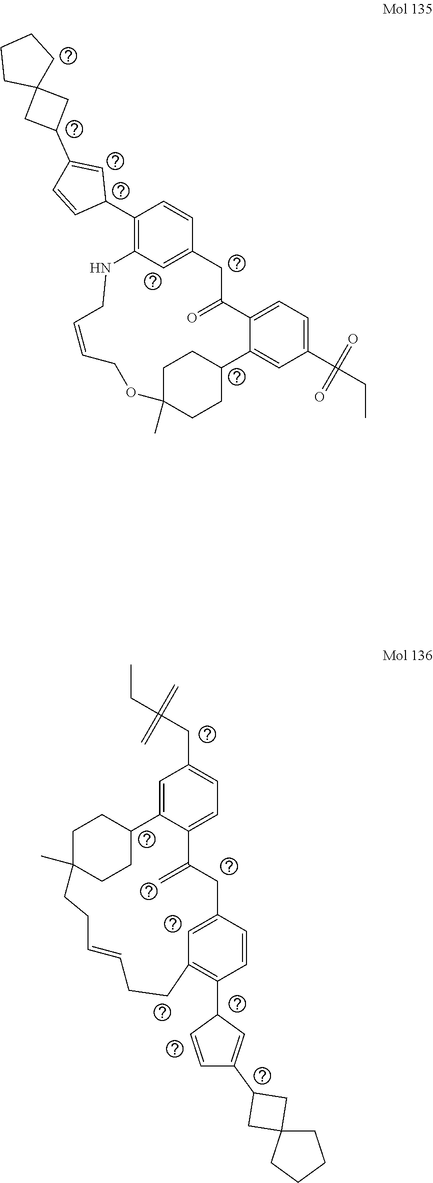

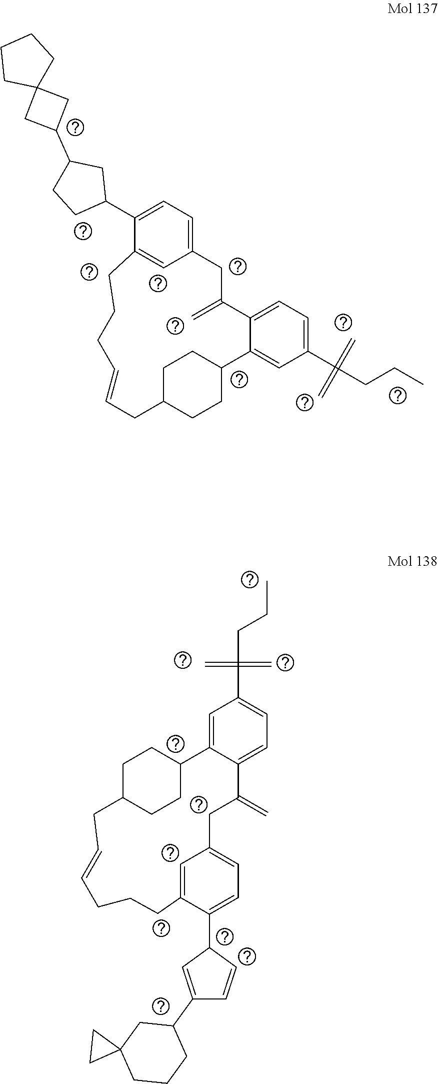

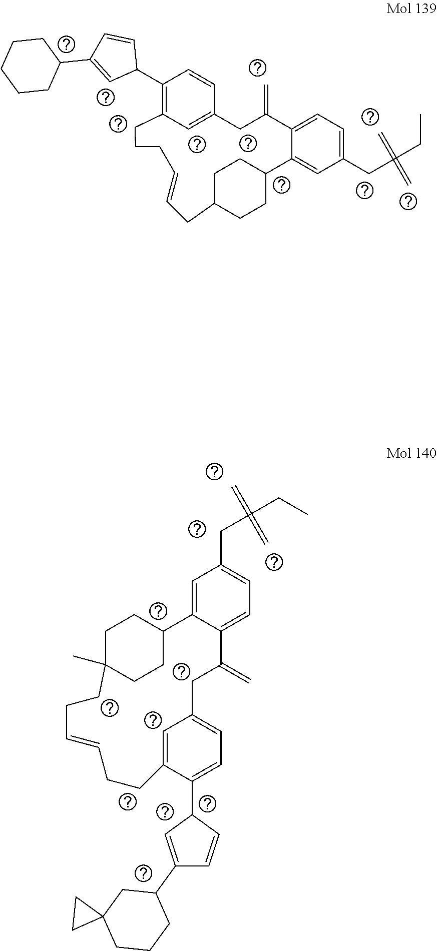

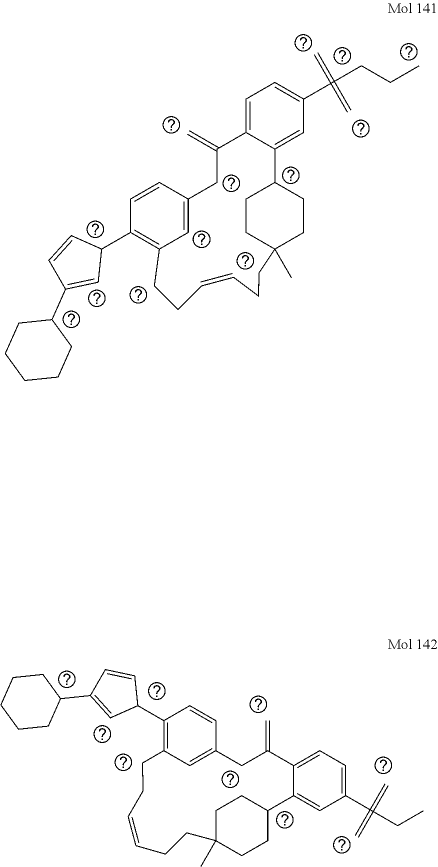

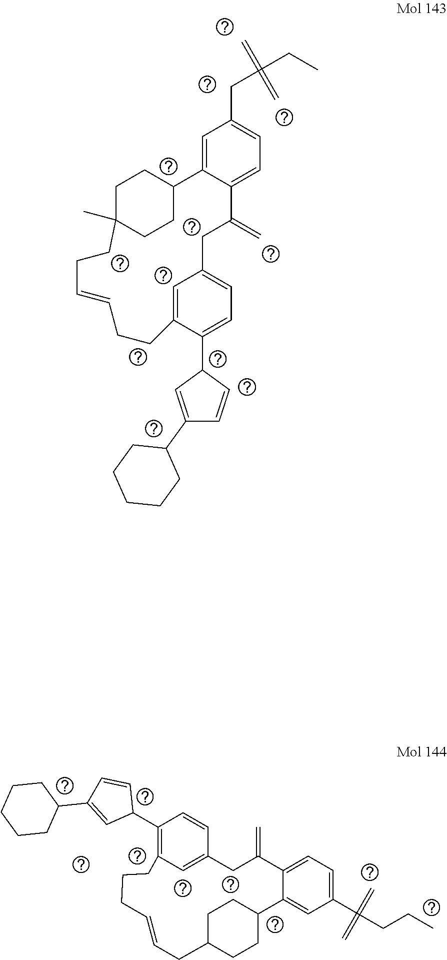

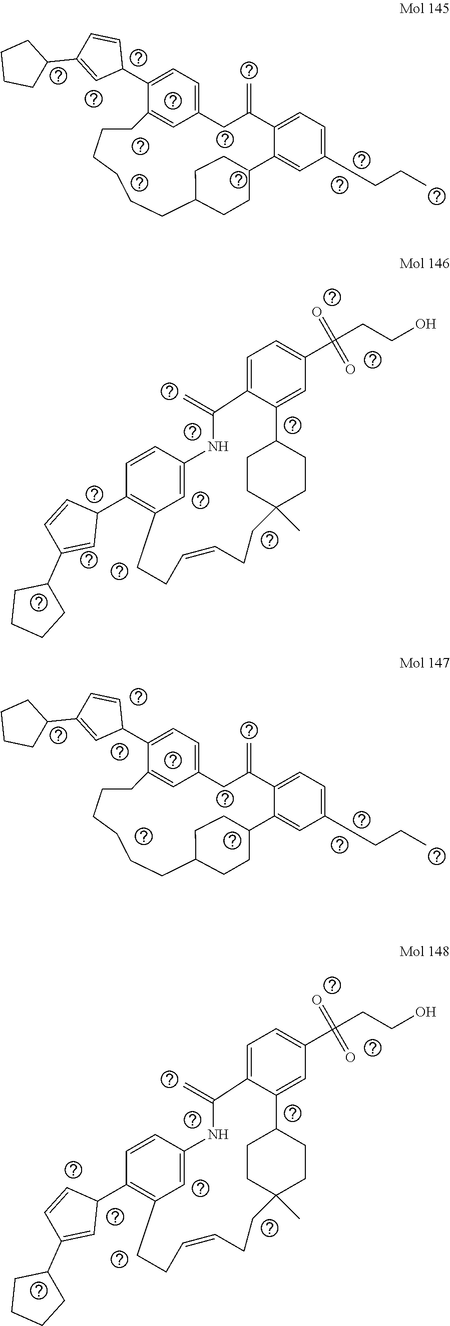

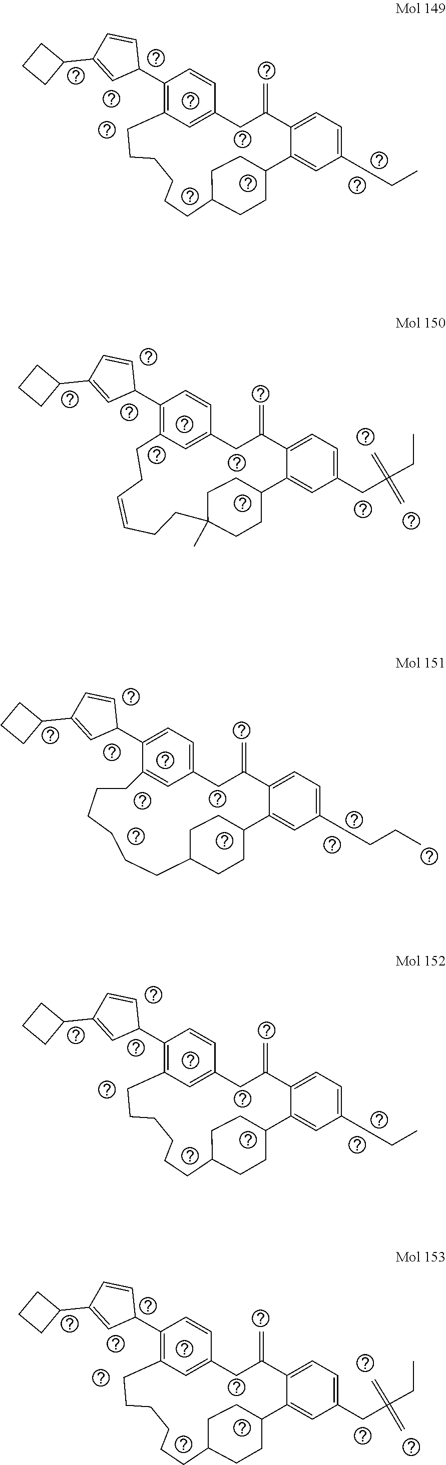

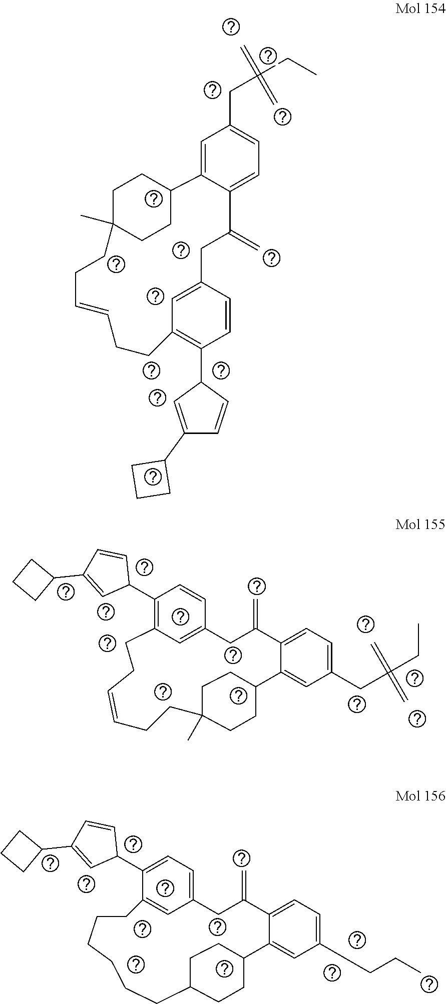

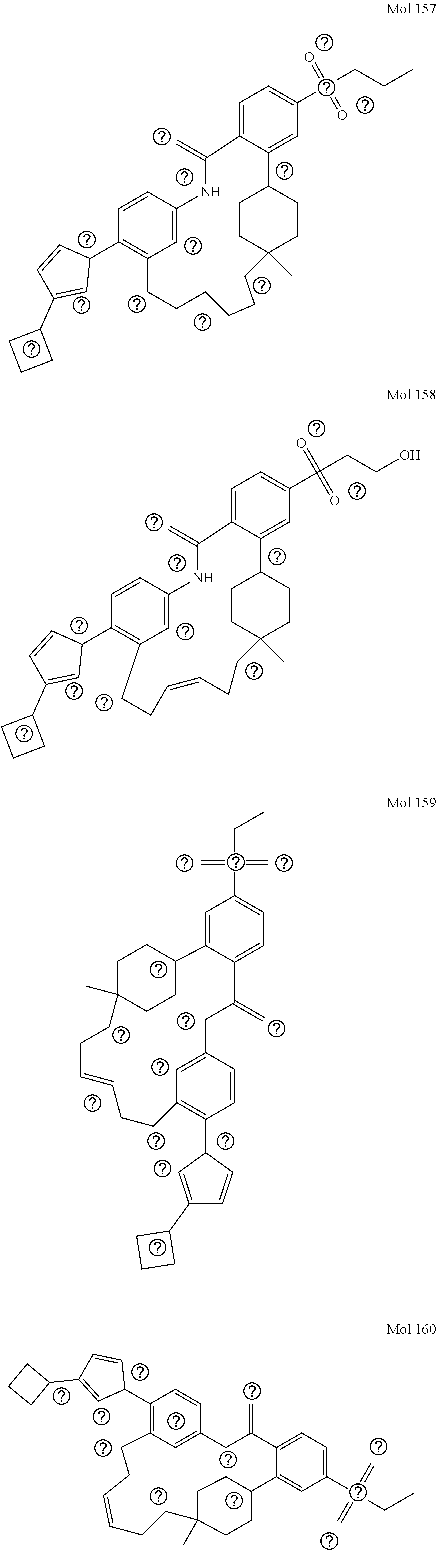

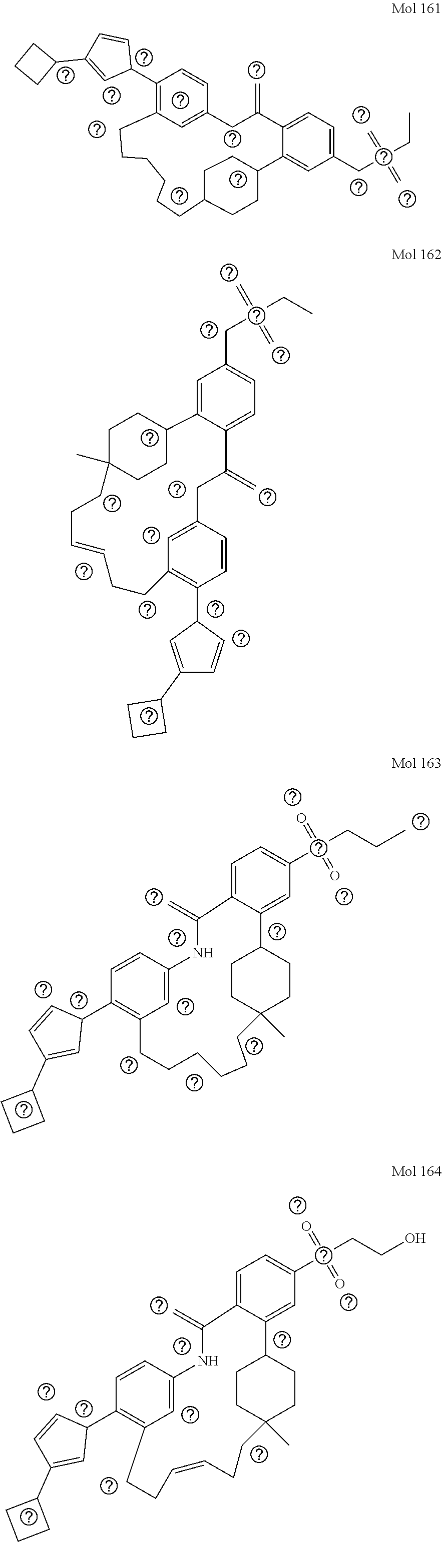

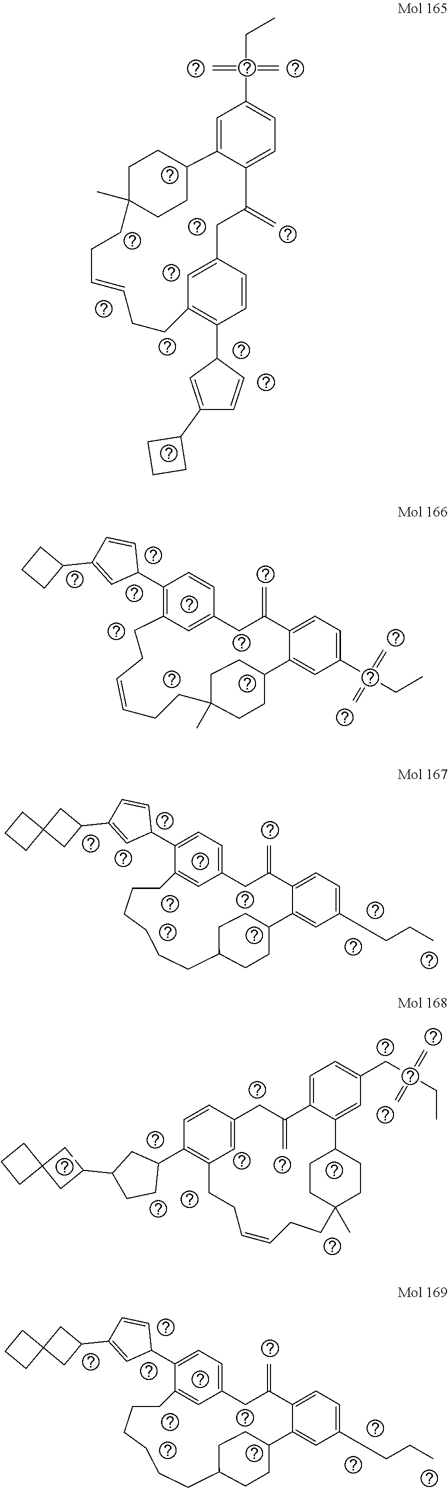

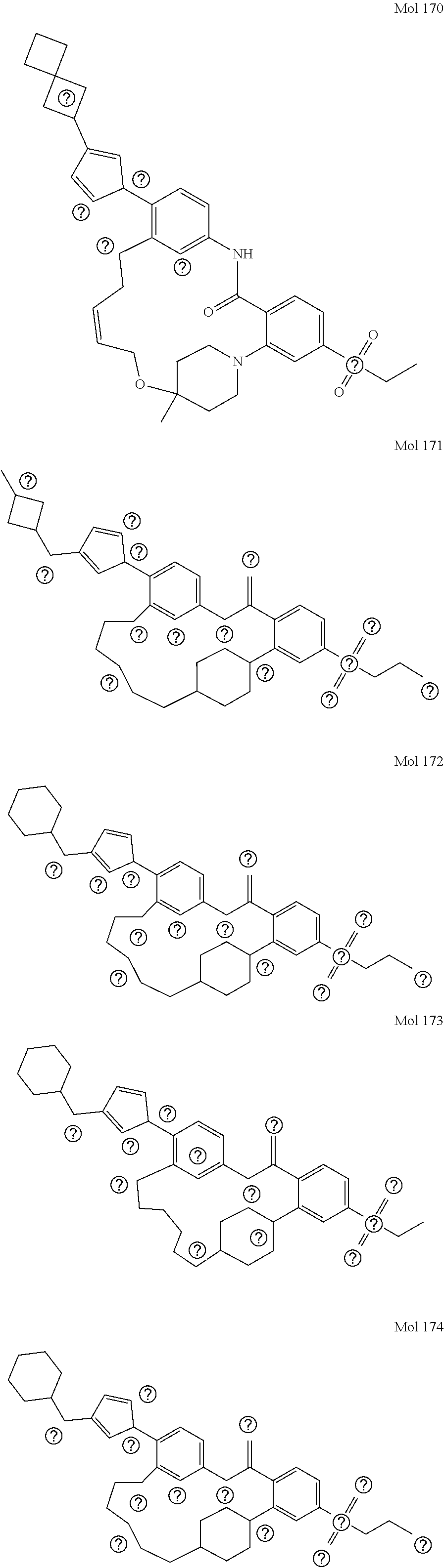

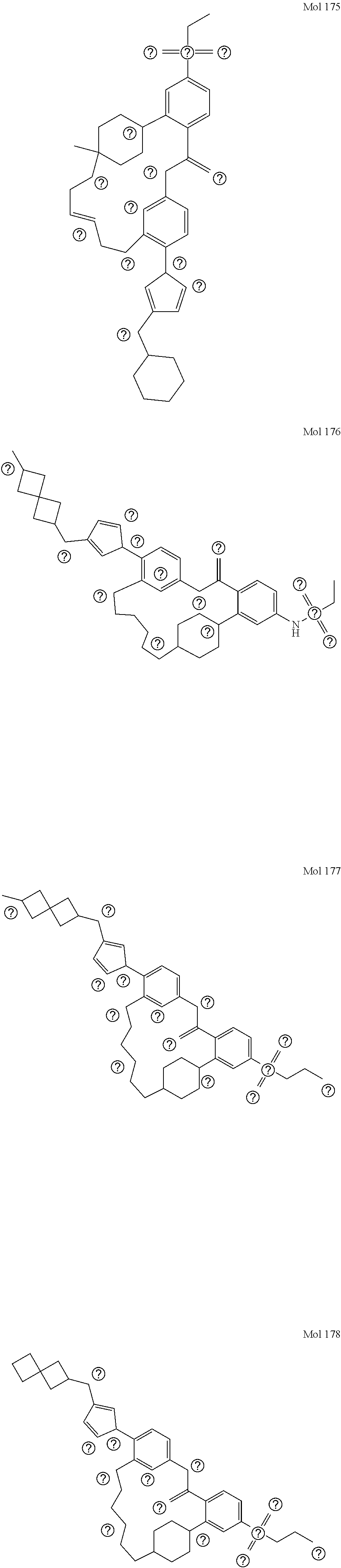

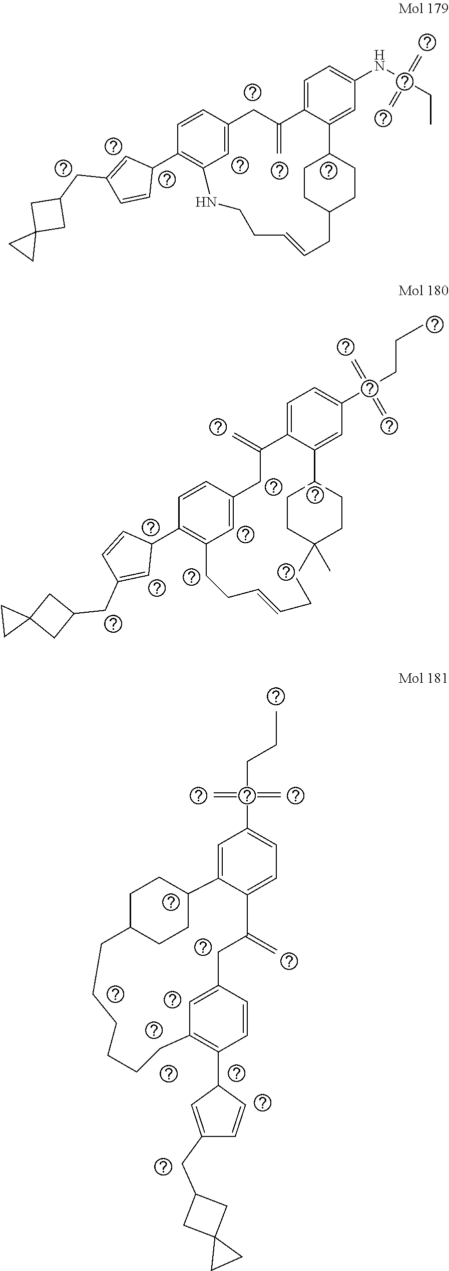

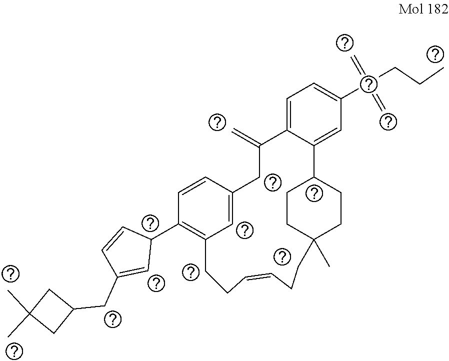

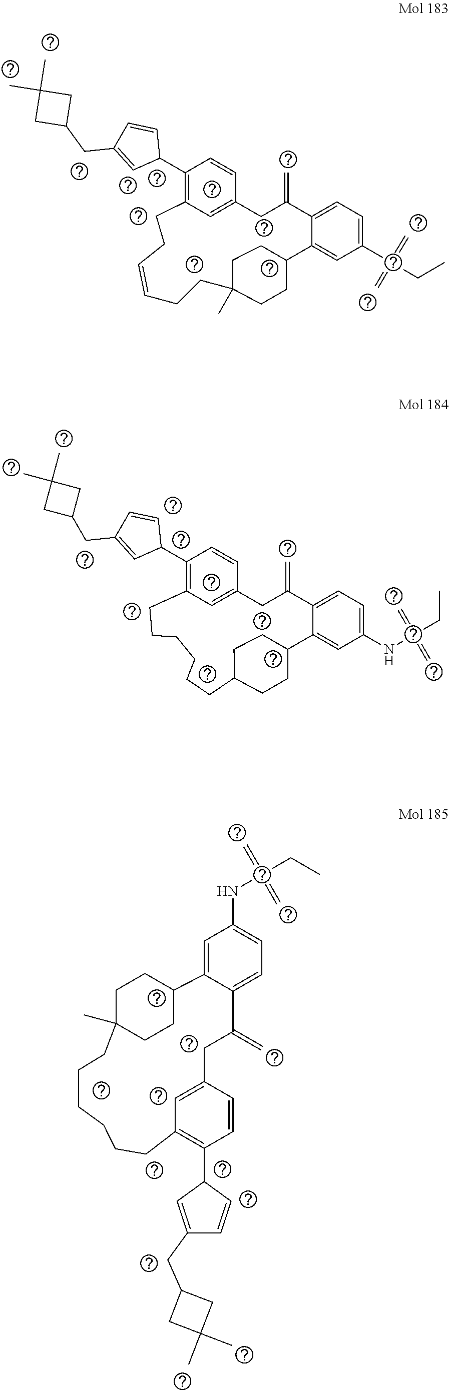

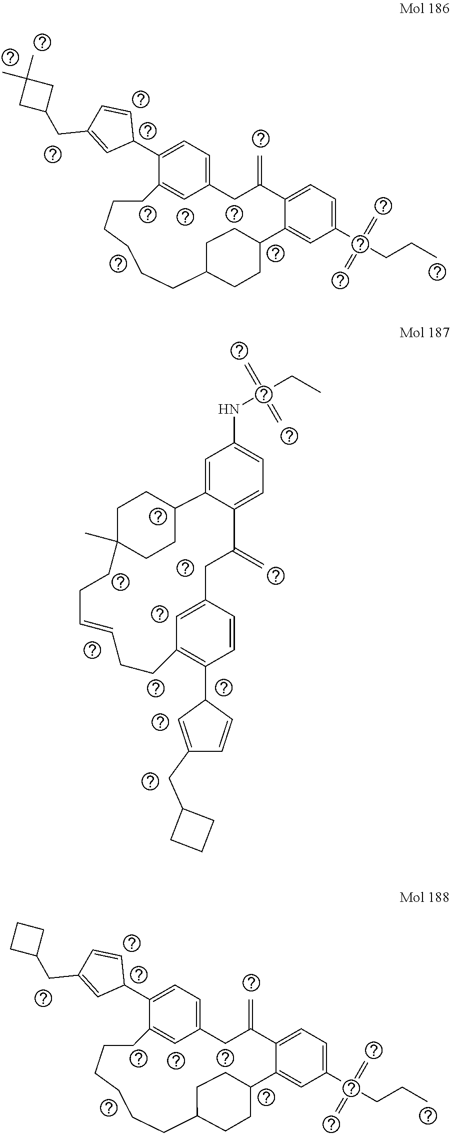

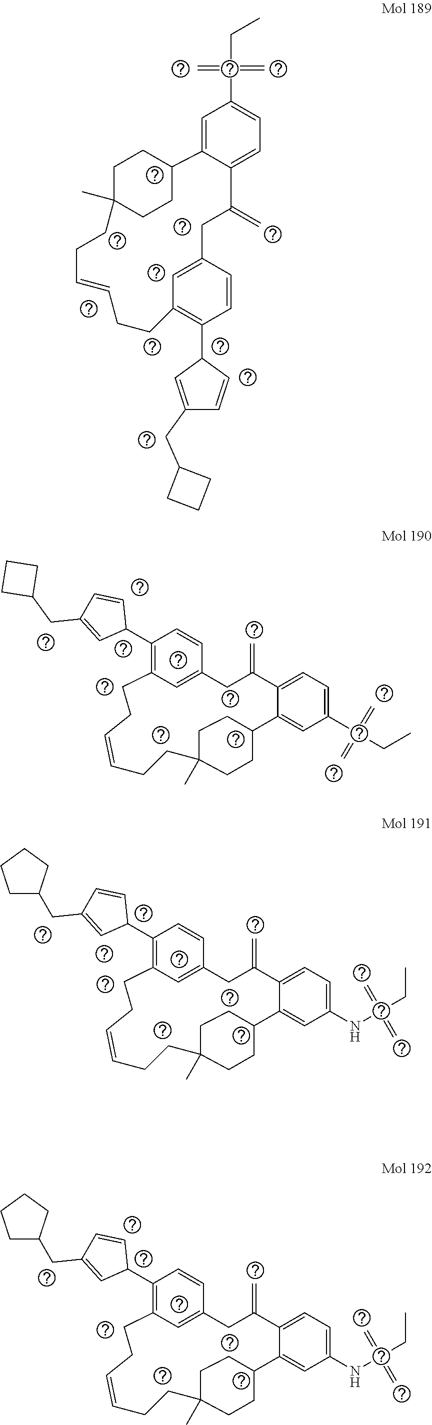

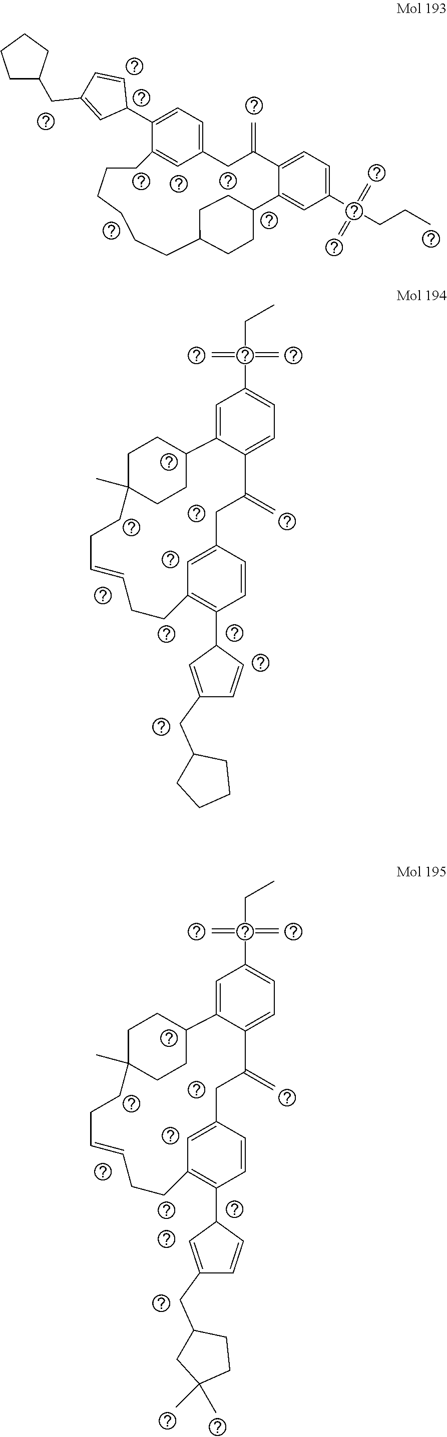

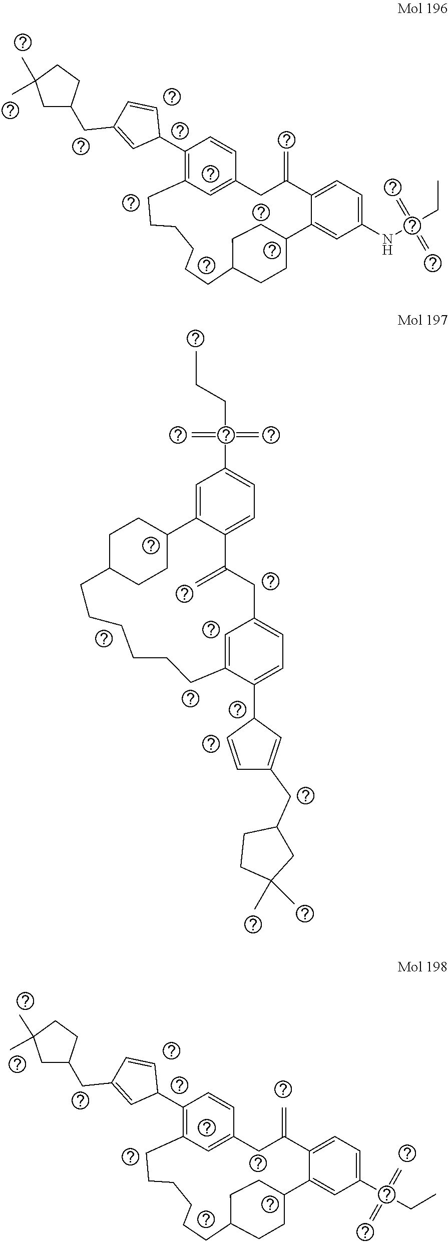

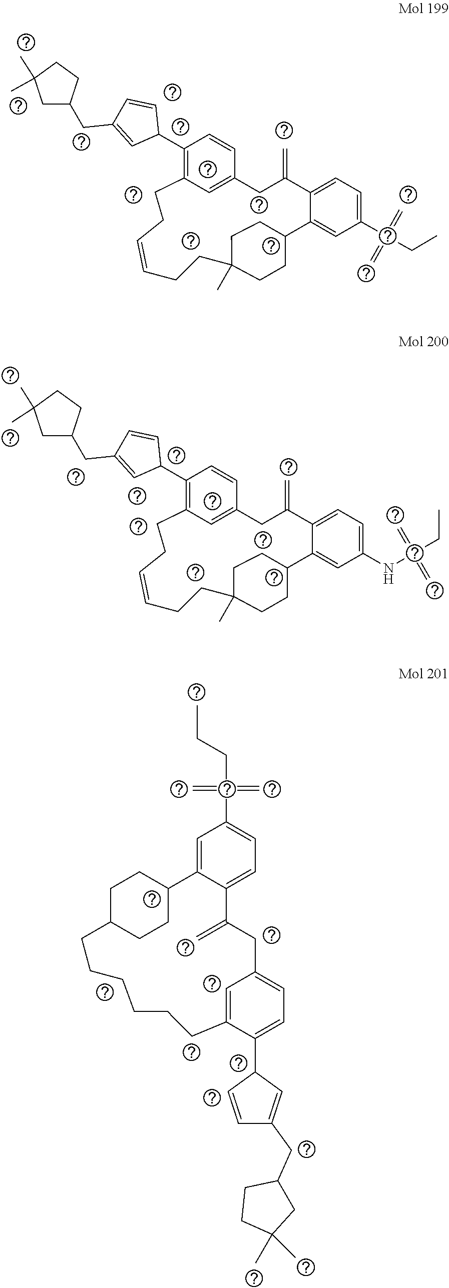

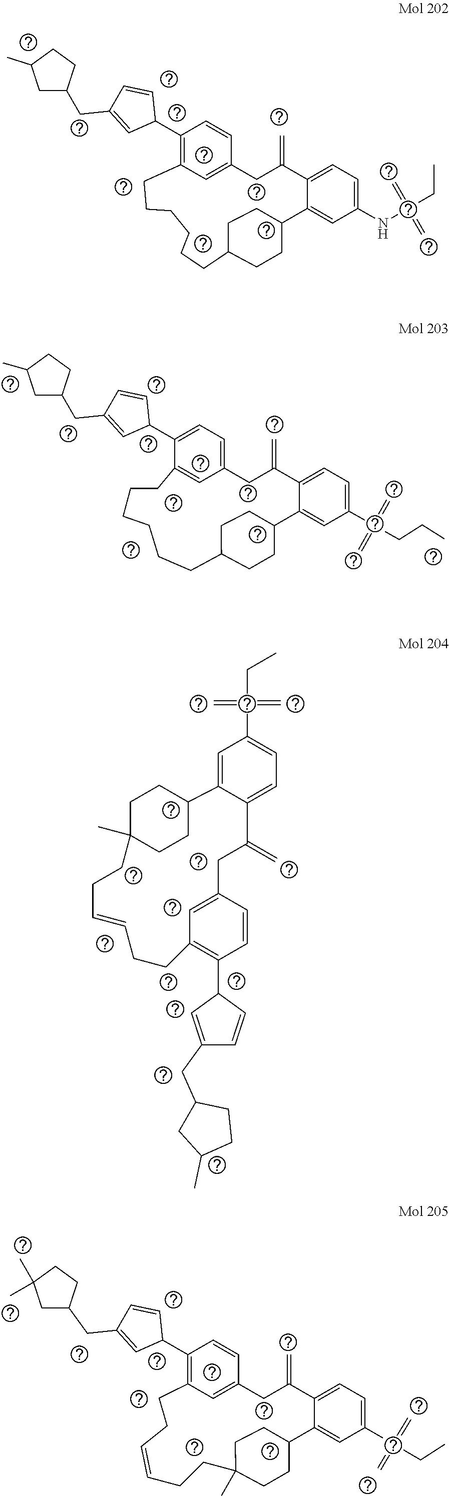

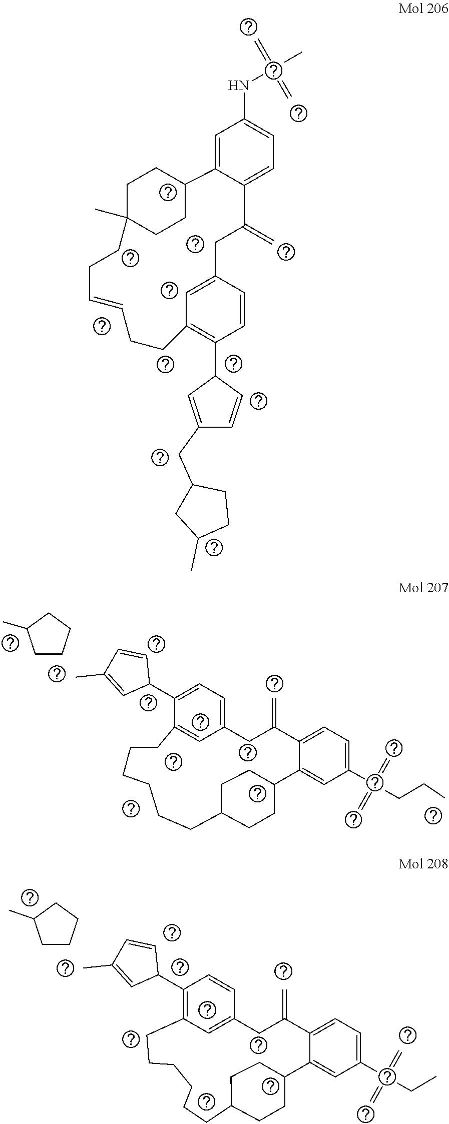

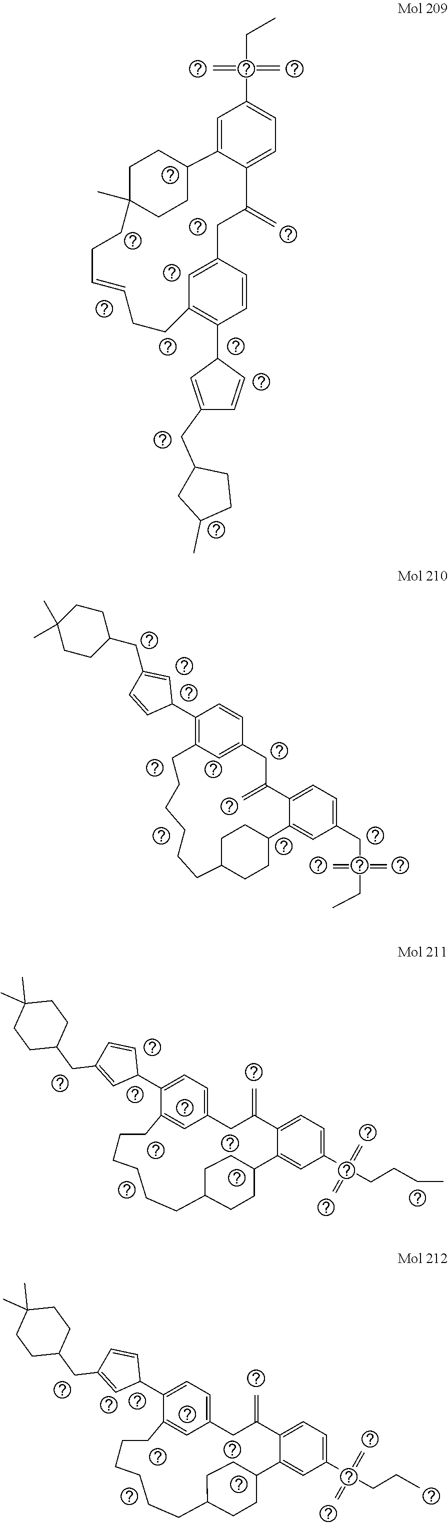

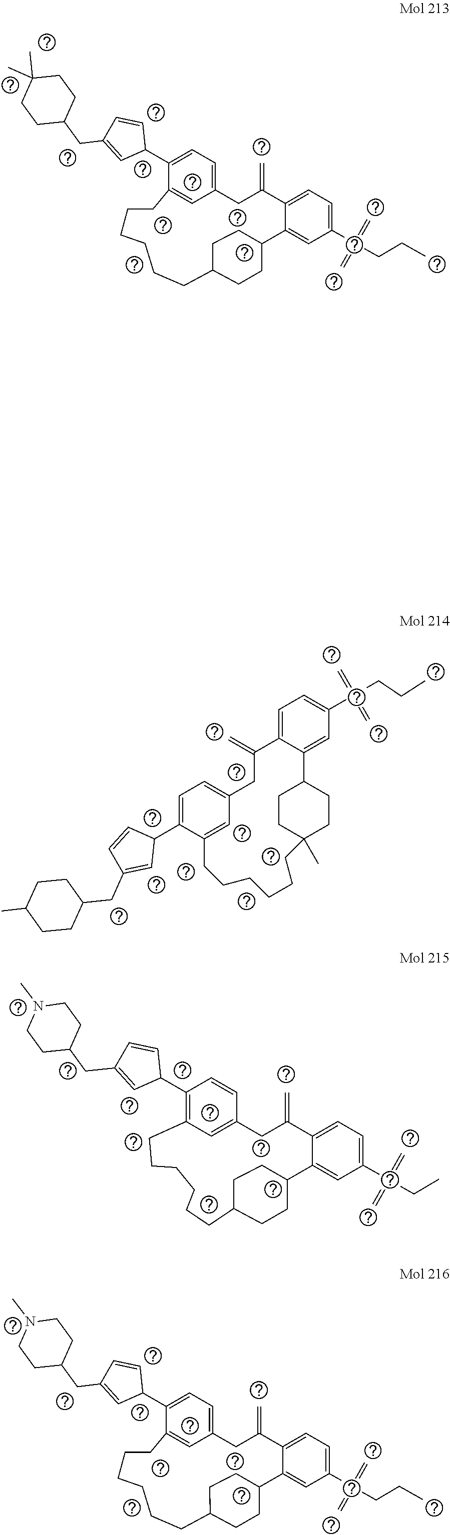

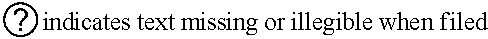

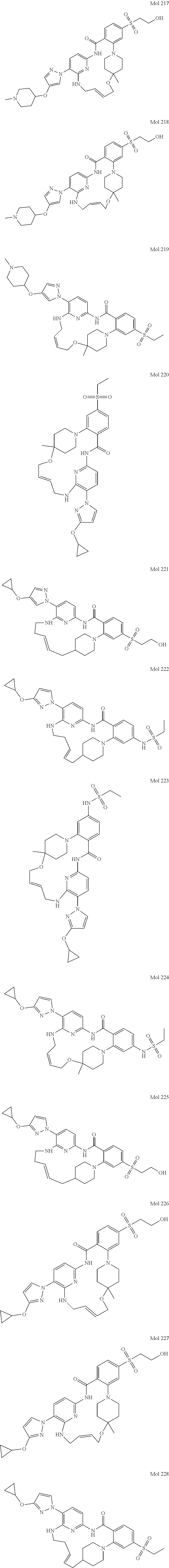

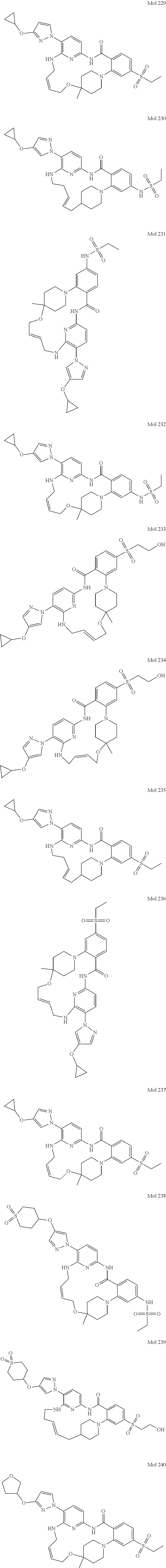

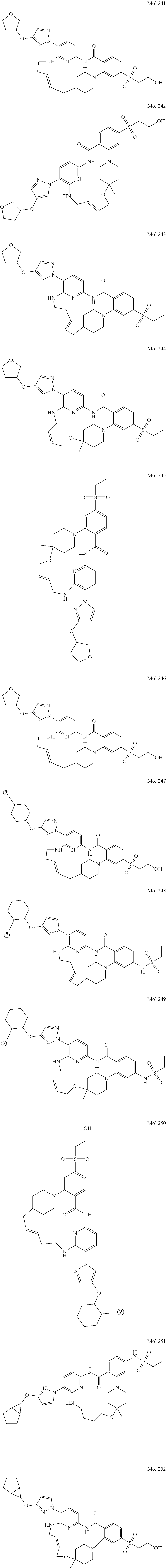

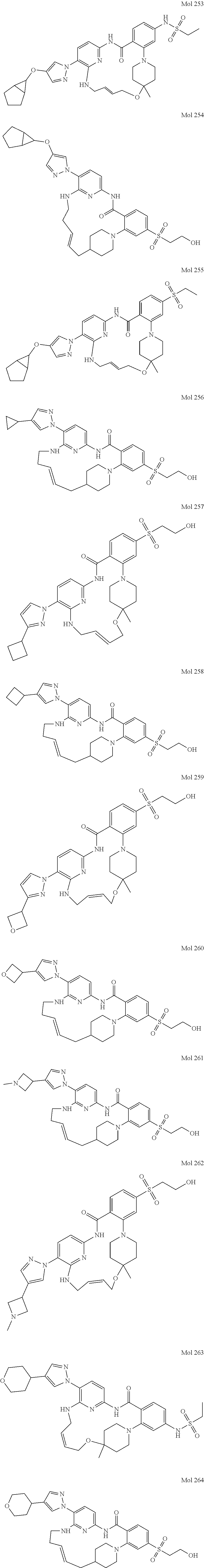

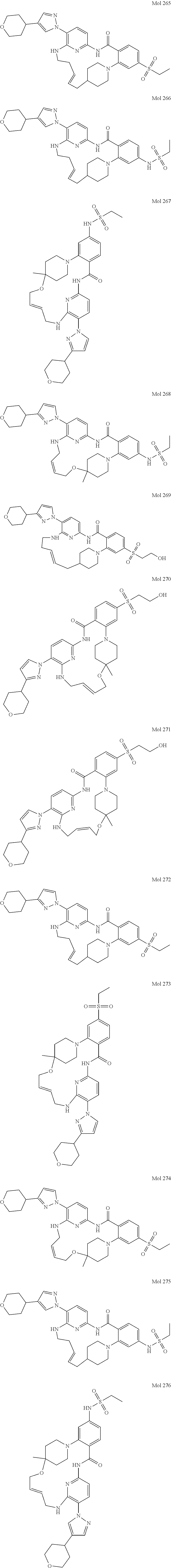

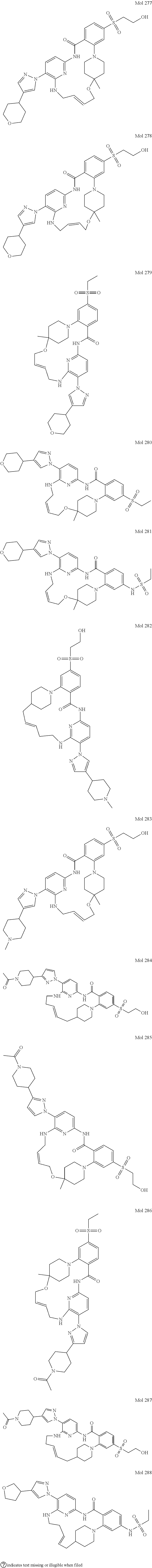

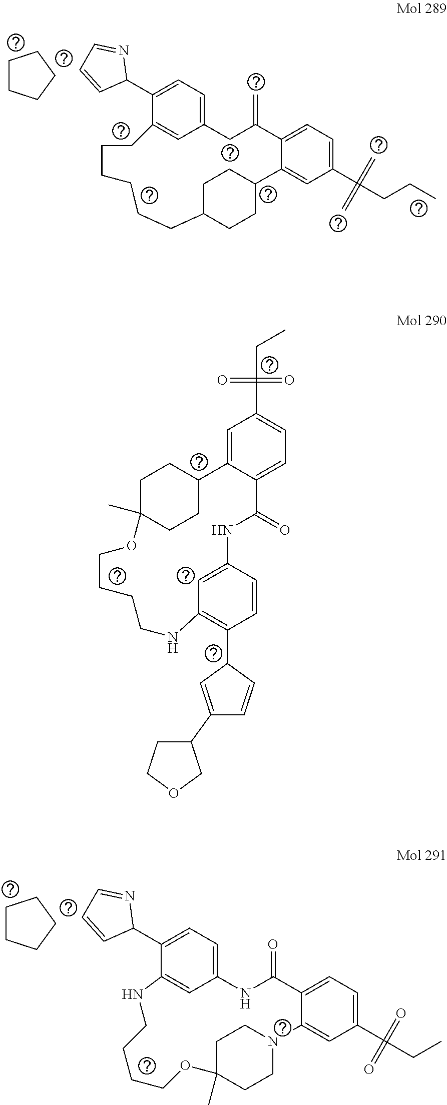

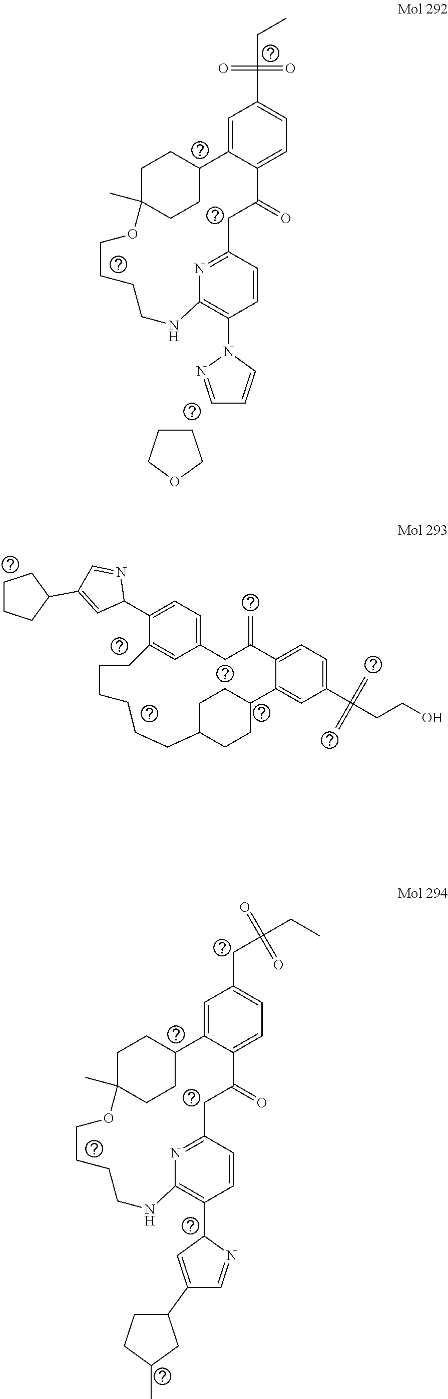

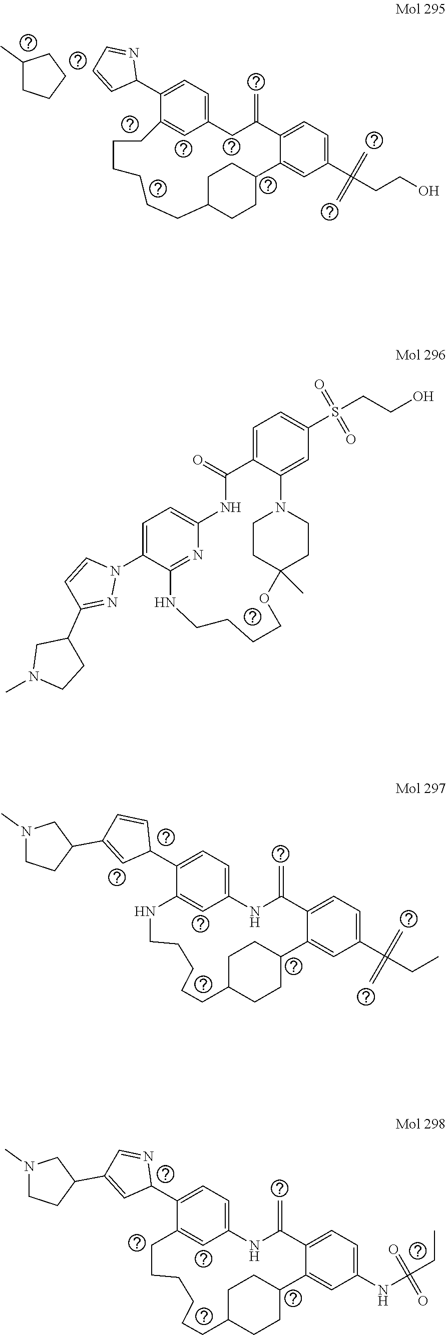

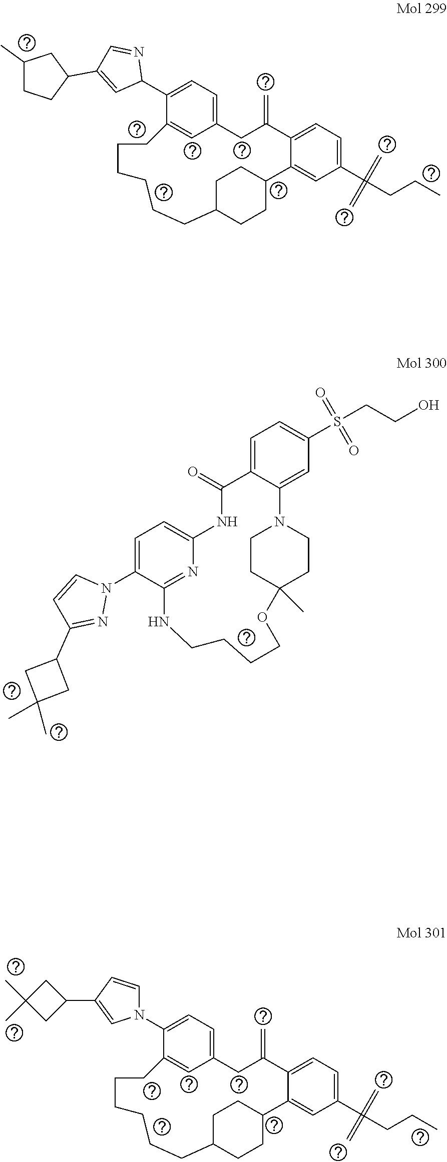

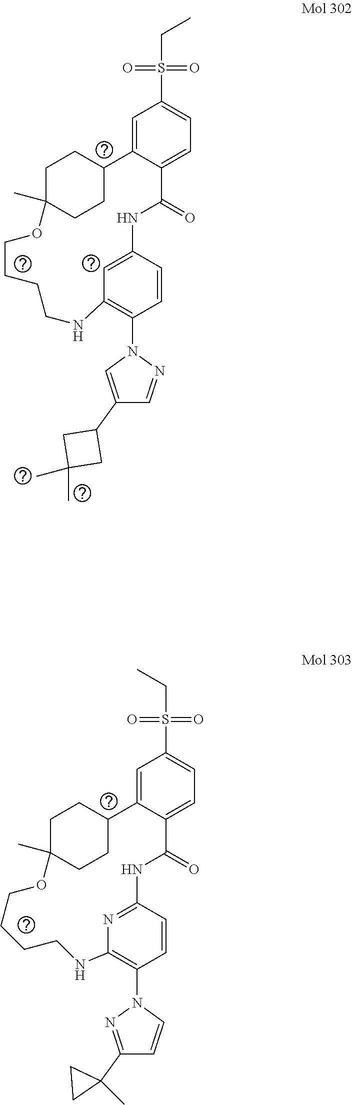 [SMILES: CCS(=O)(=O)c1ccc2c(c1)C1CCC(C)(CC1)OCCCCNc1cc(ccc1-n1cc(C3CC(C)(C)C3)cn1)NC2=O ; CCS(=O)(=O)c1ccc2c(c1)C1CCC(C)(CC1)OCCCCNc1nc(ccc1-n1ccc(C3(C)CC3)n1)NC2=O]

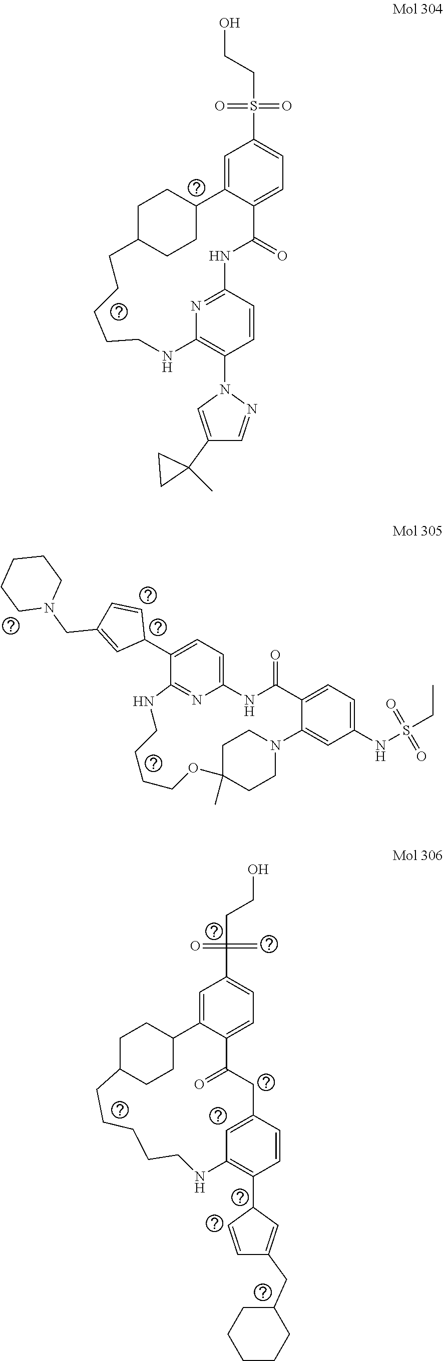

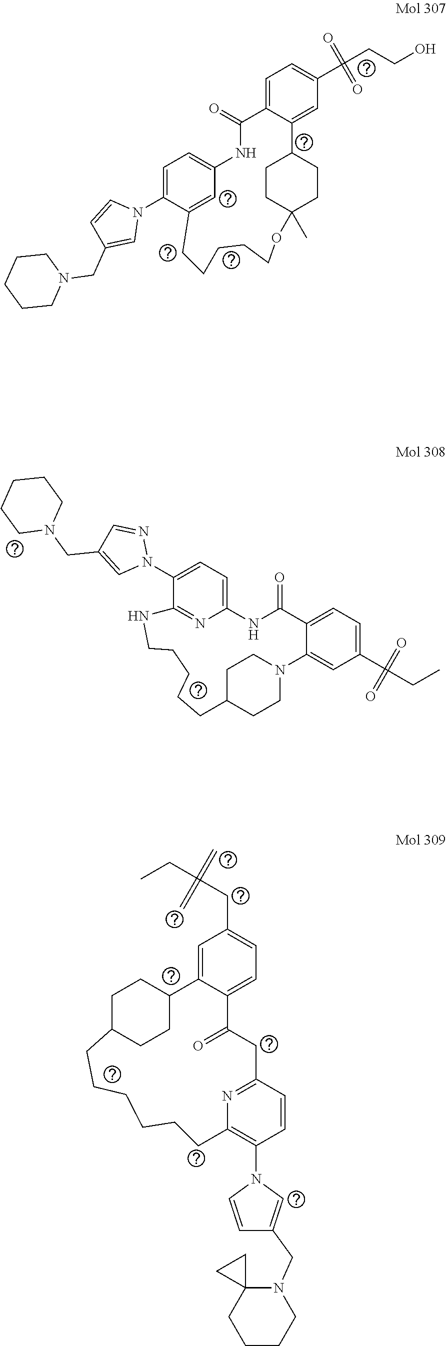

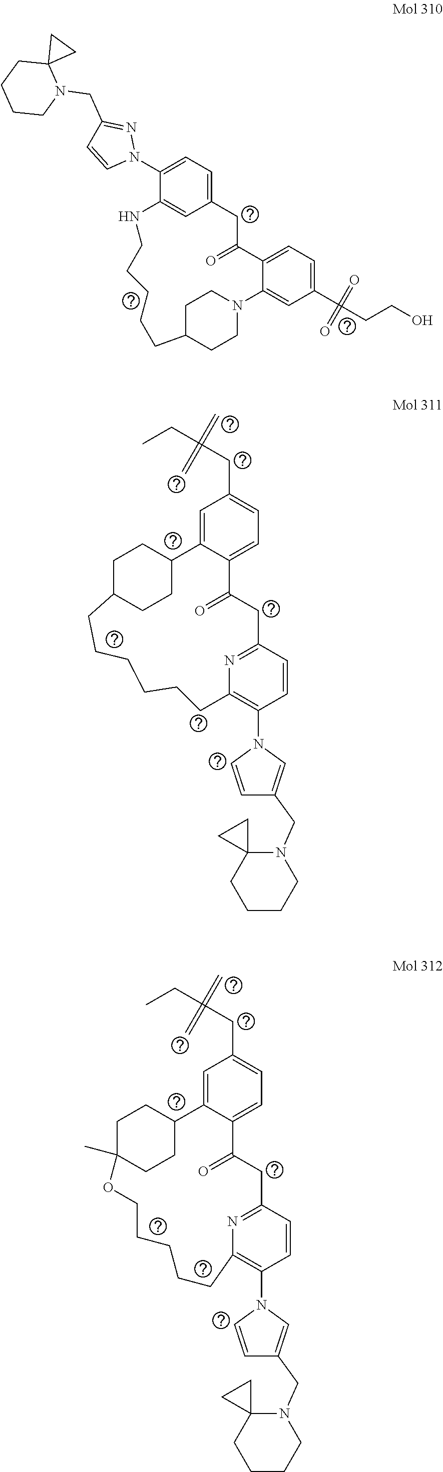

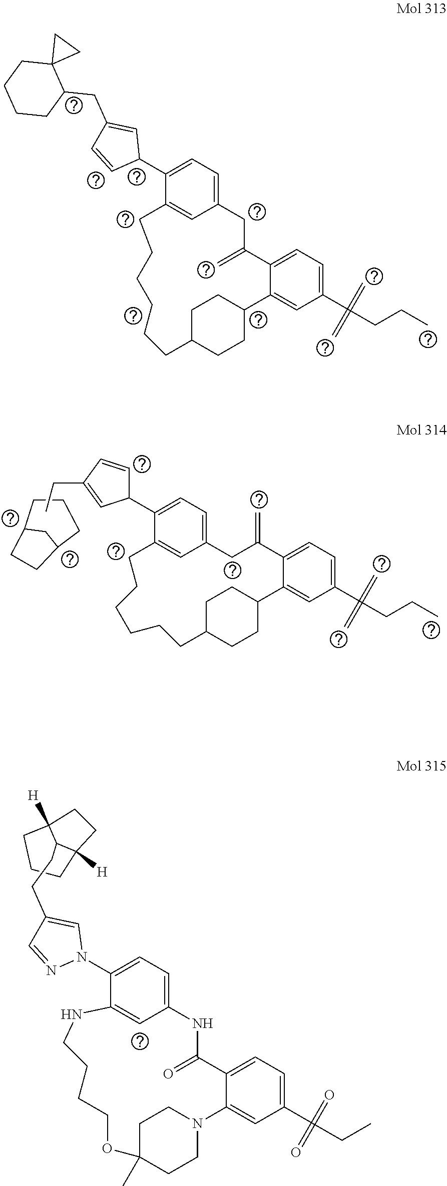

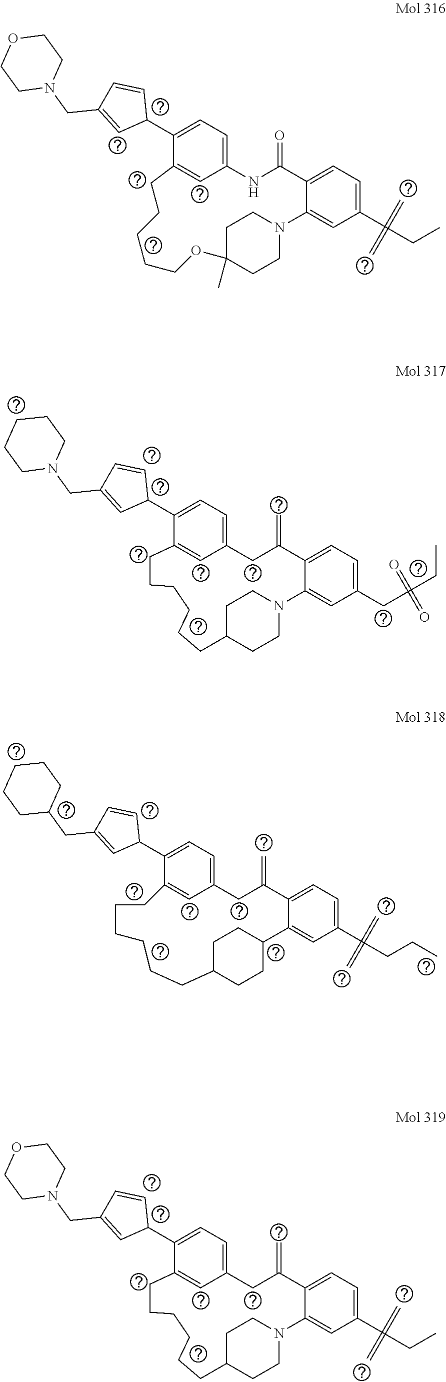

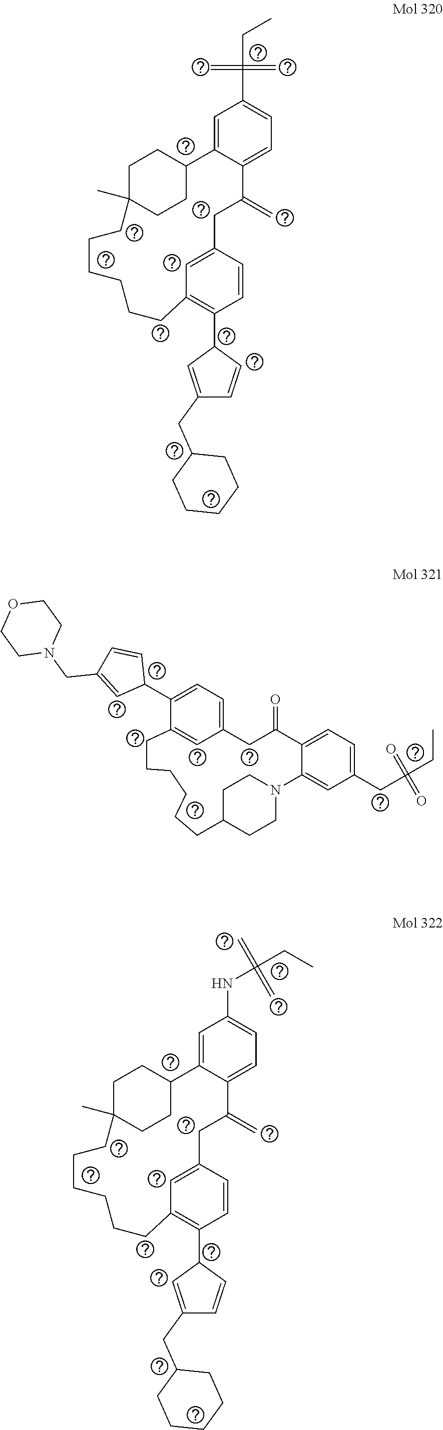

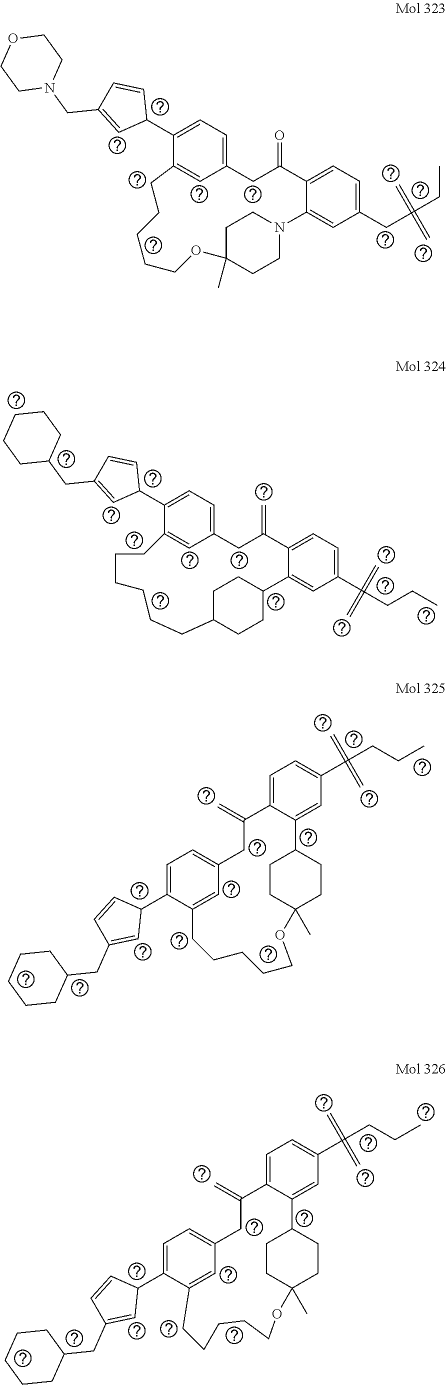

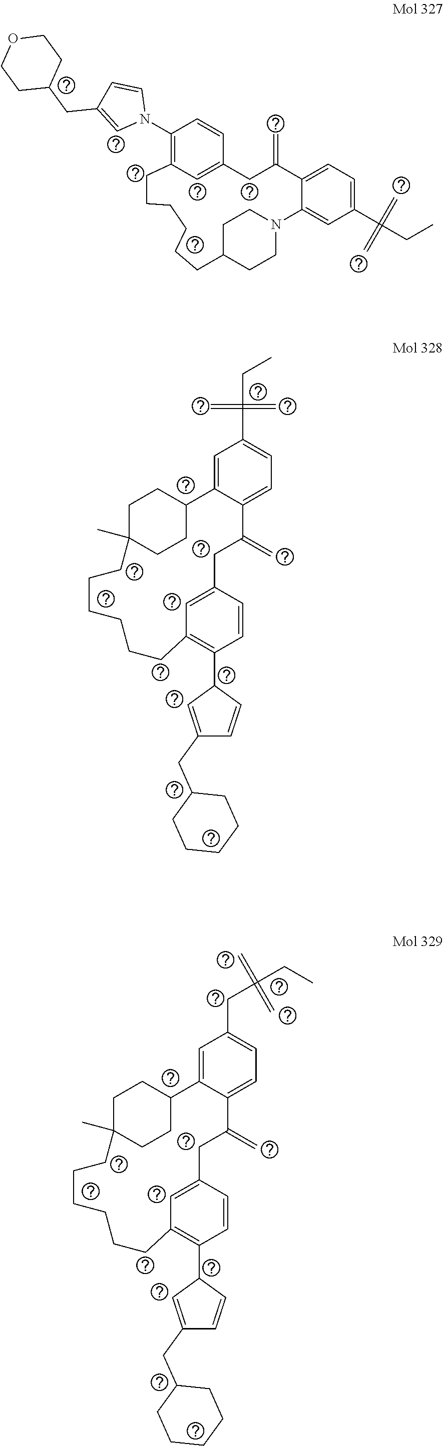

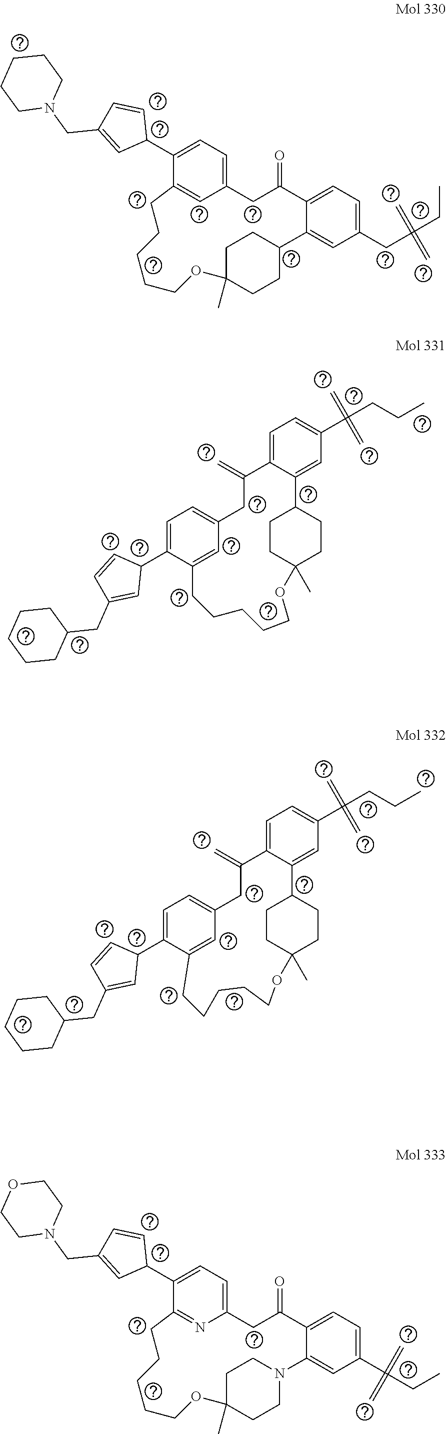

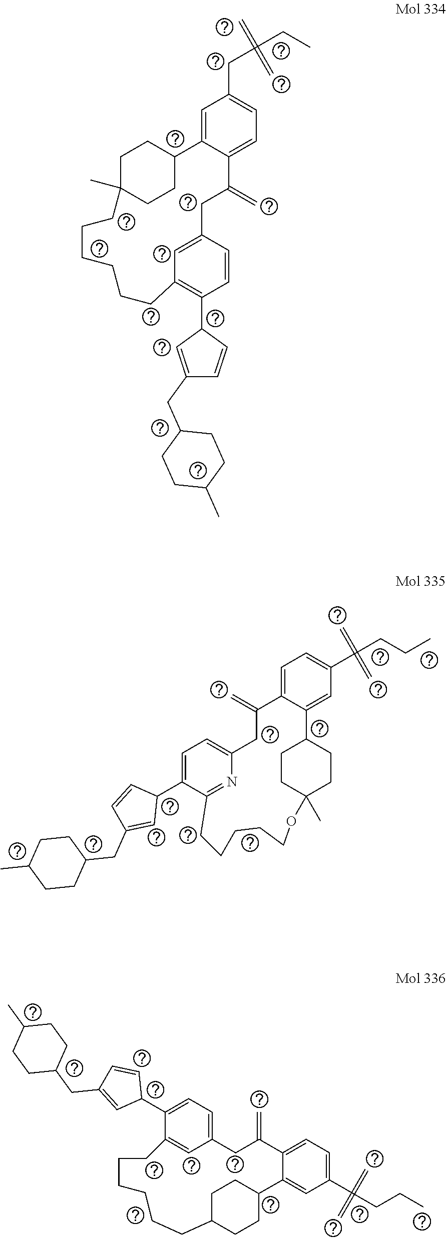

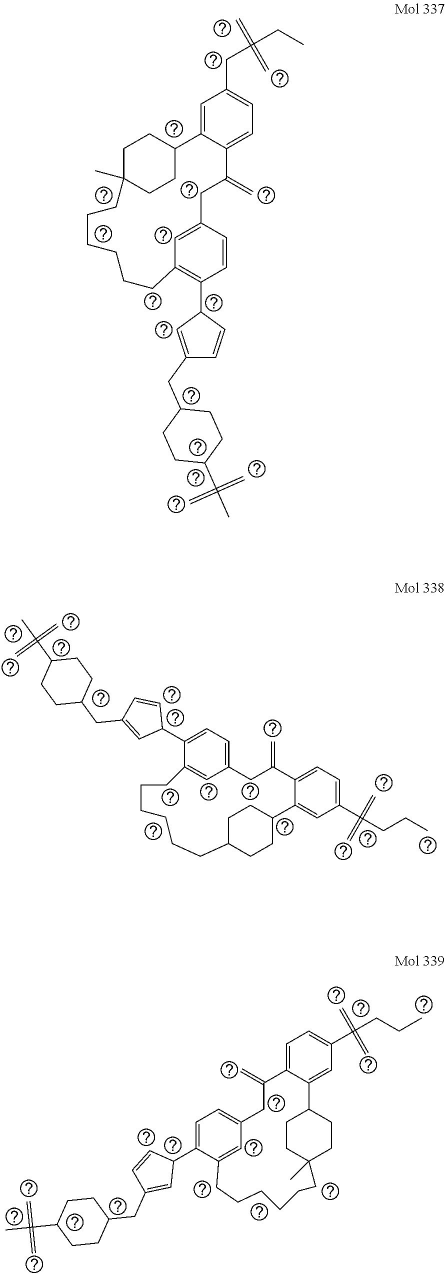

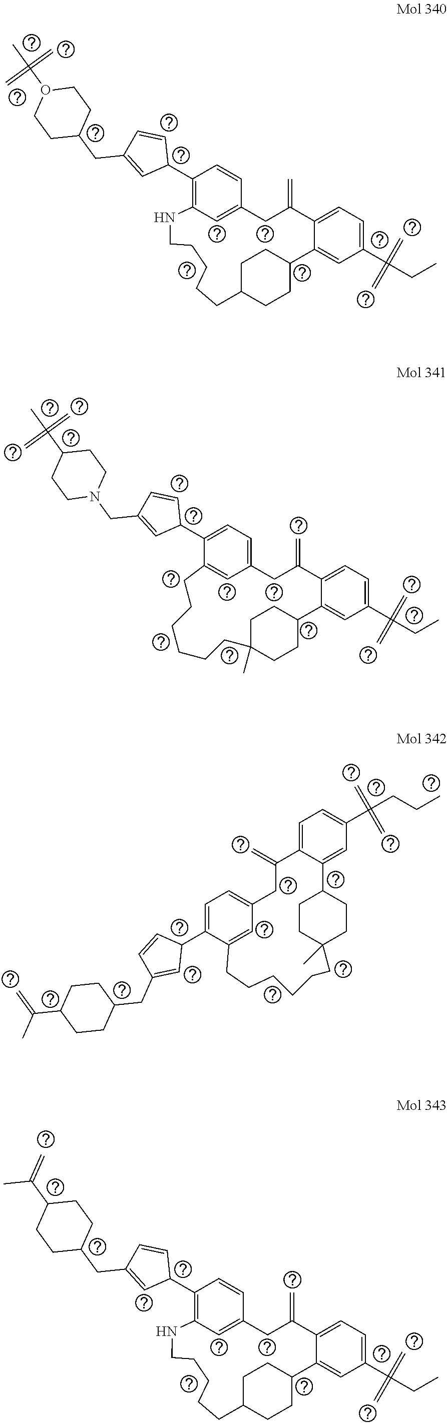

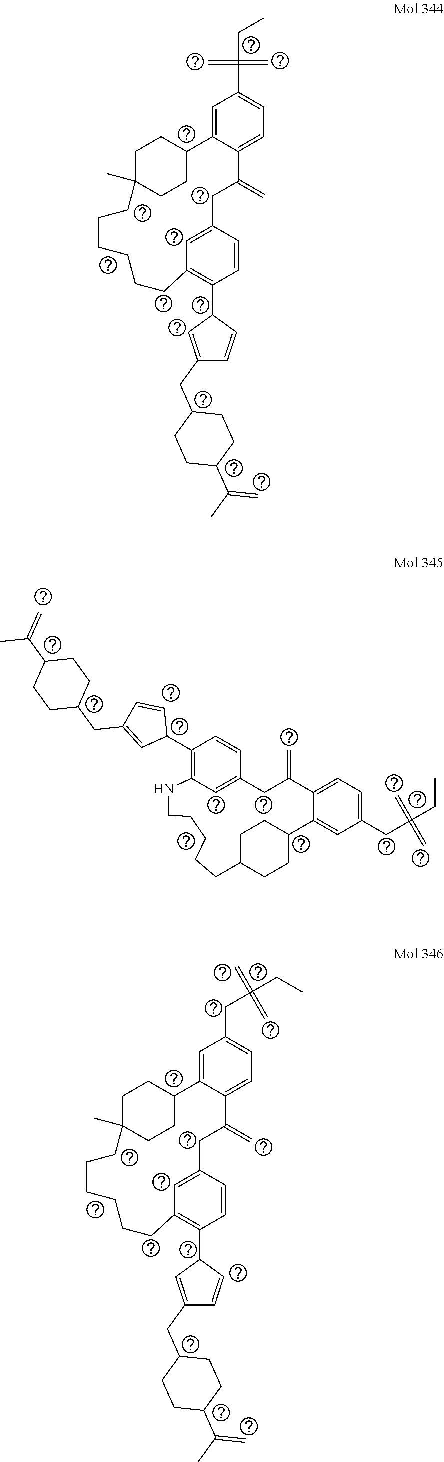

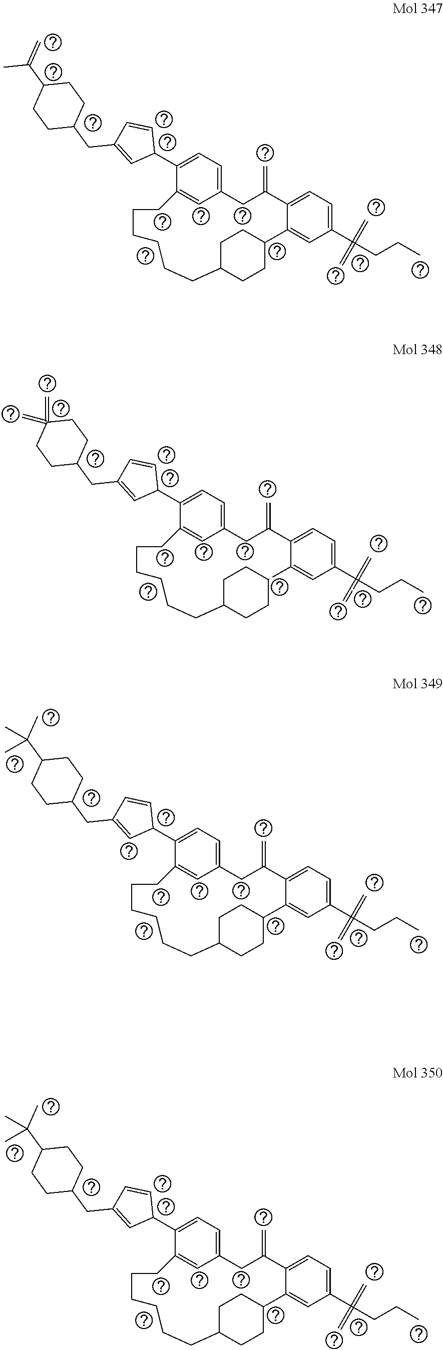

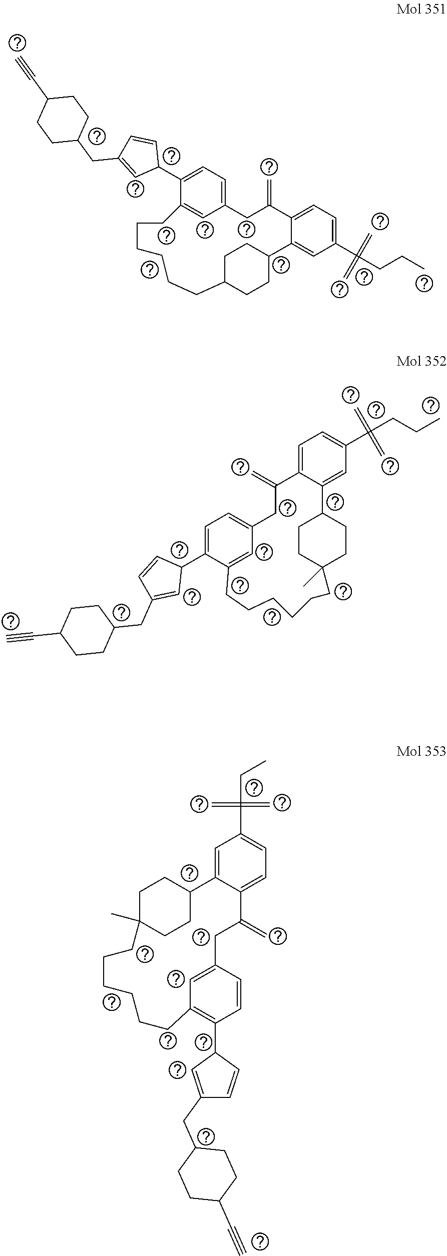

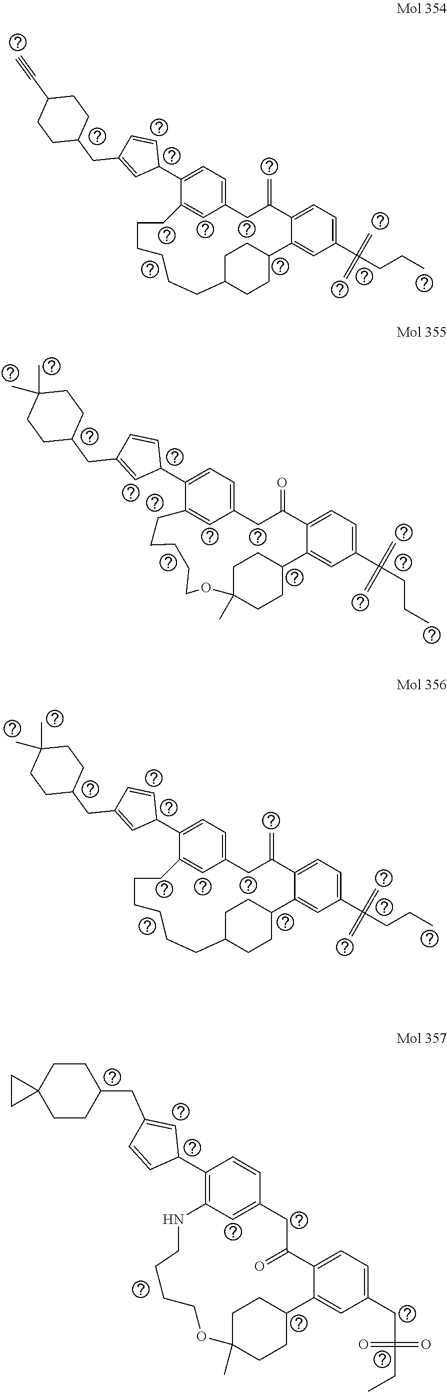

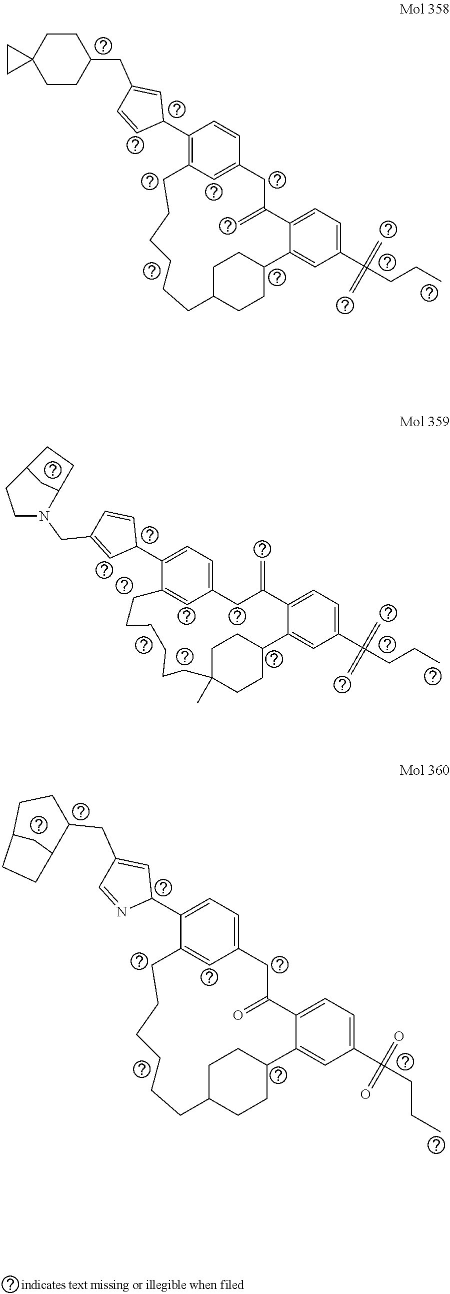

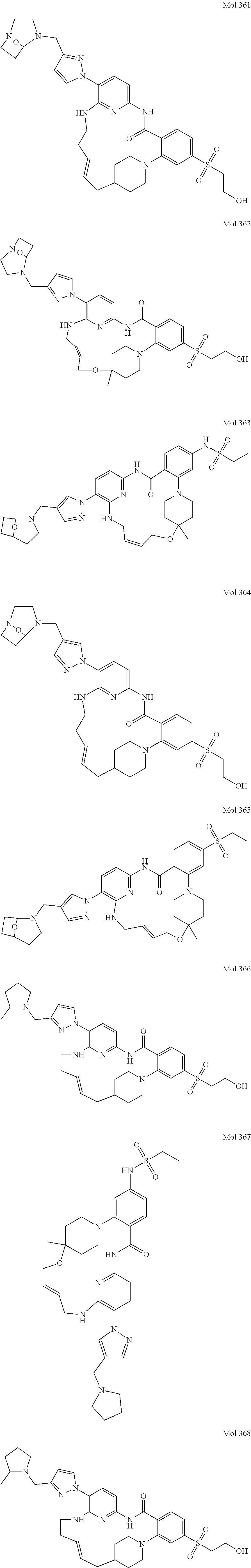

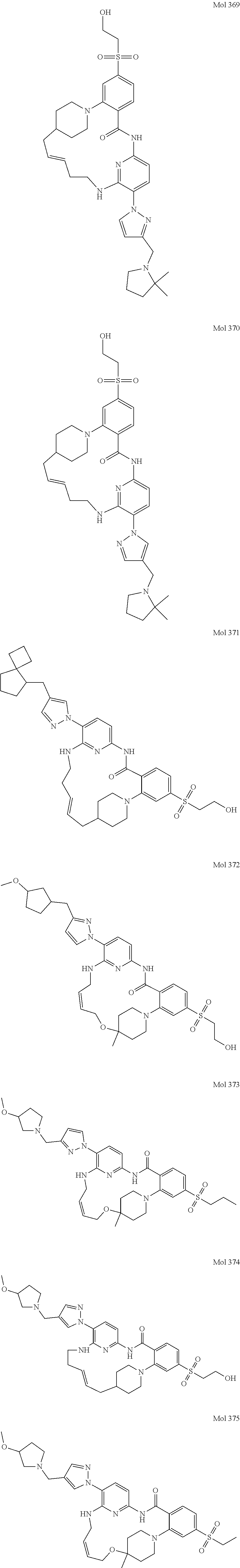

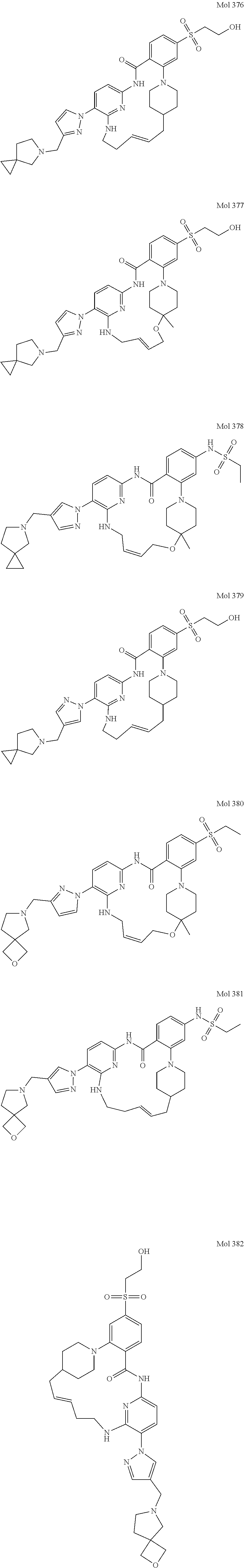

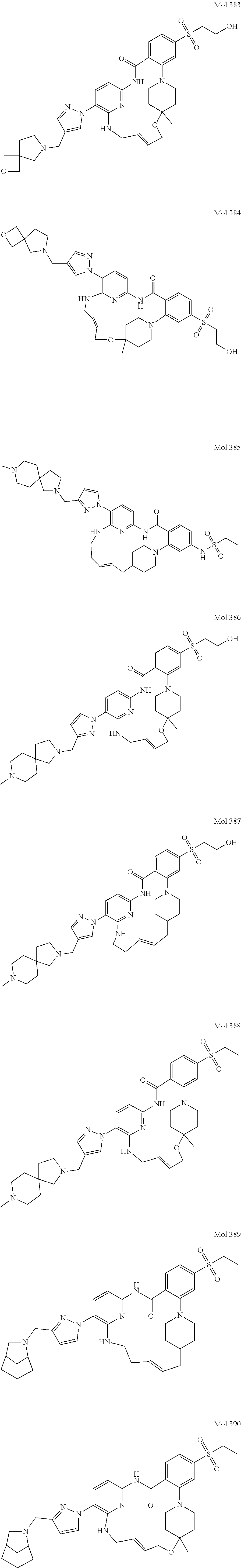

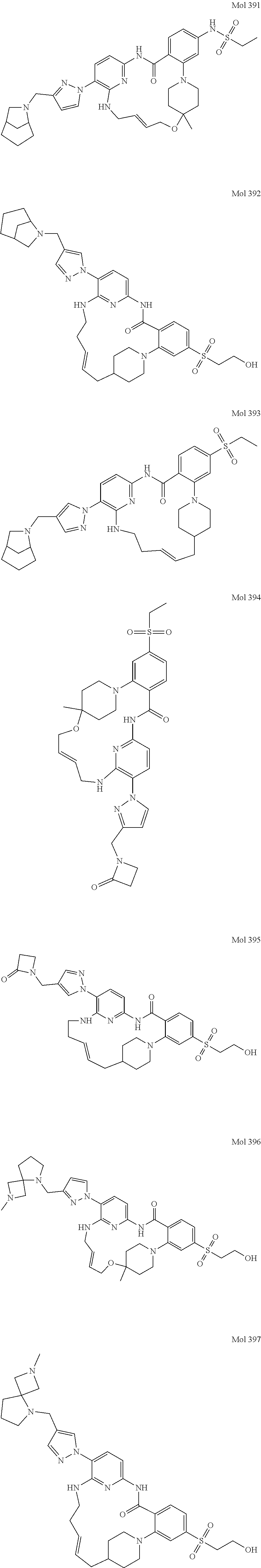

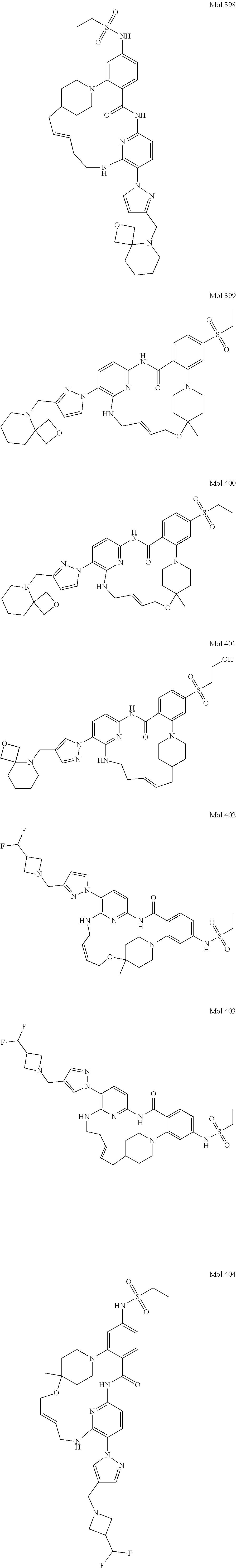

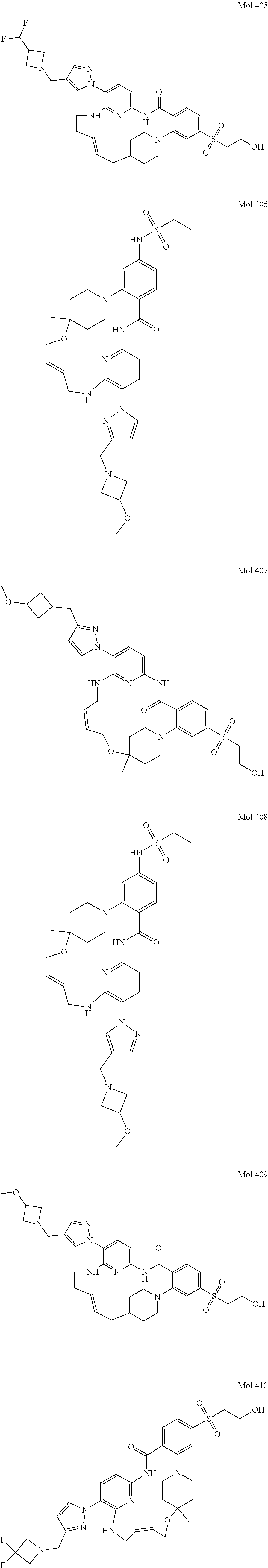

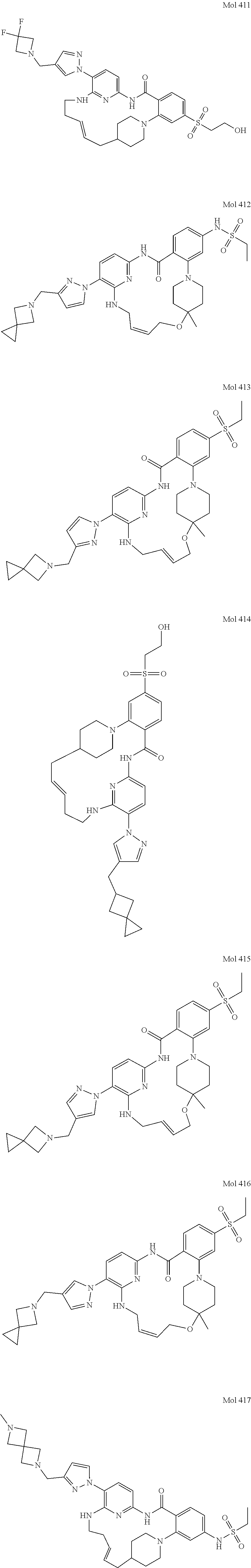

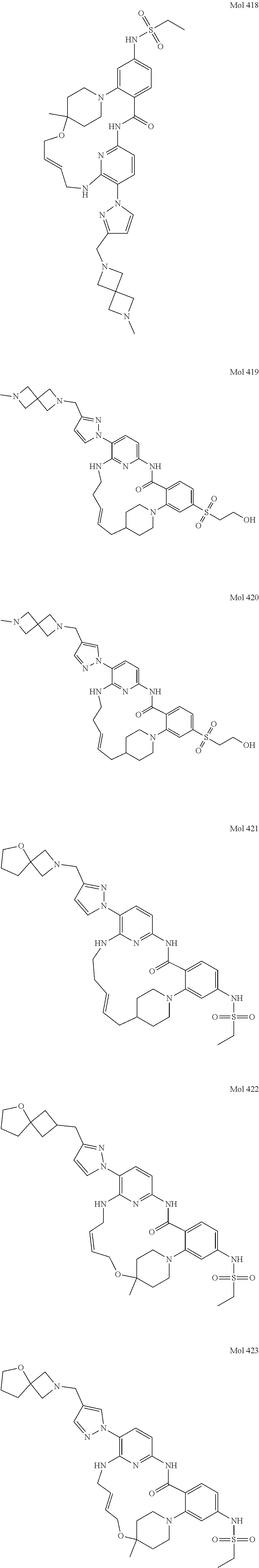

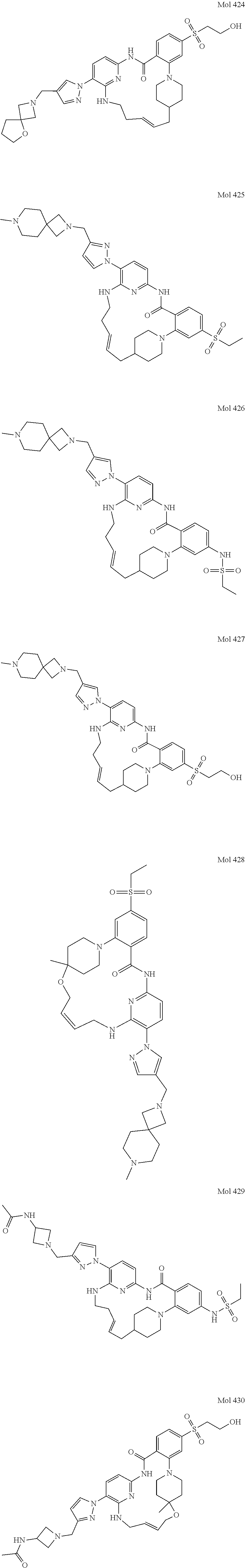

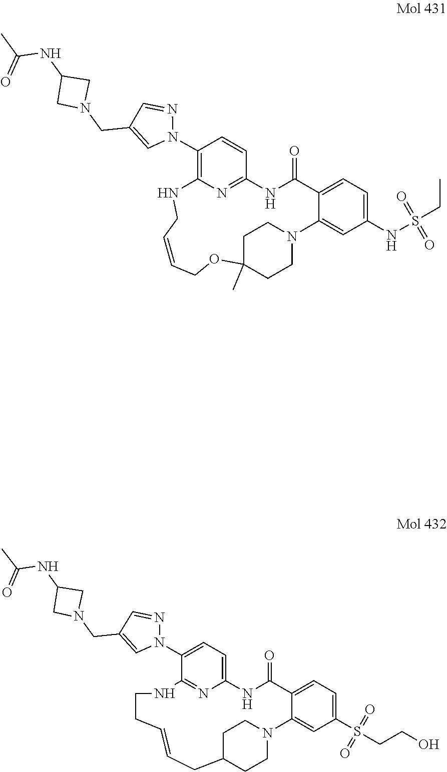

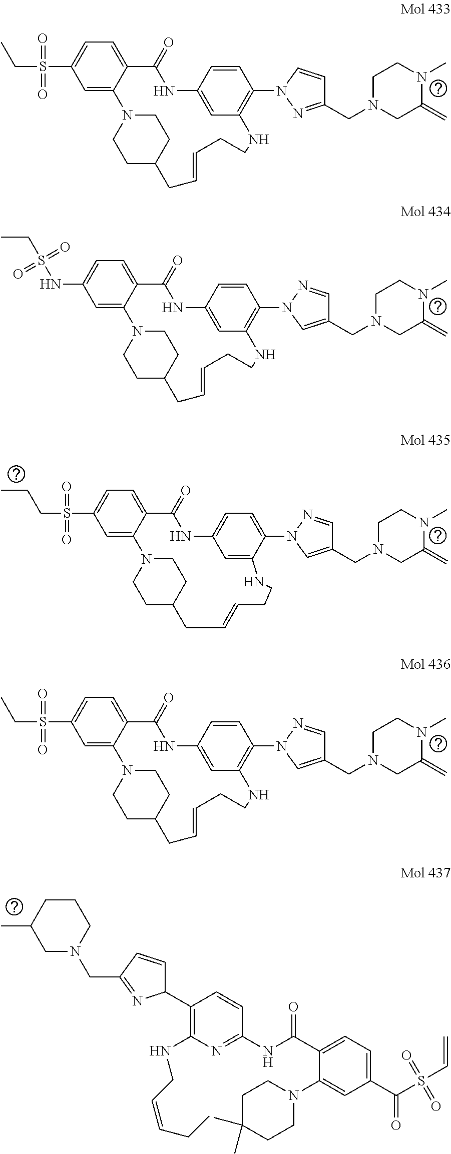

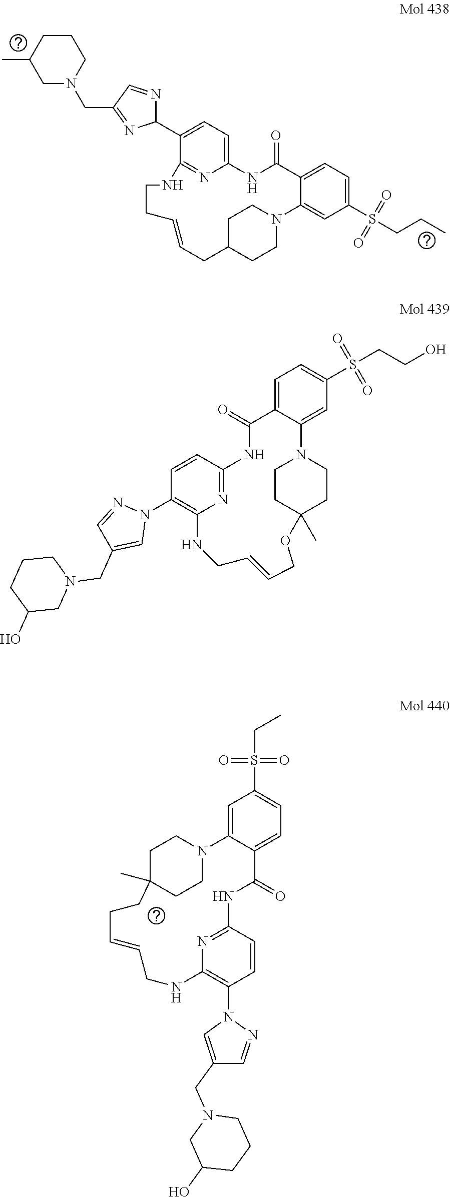 [SMILES: CCCS(=O)(=O)c1ccc2c(c1)N1CCC(C/C=C/CCNc3nc(ccc3C3N=CC(CN4CCCC(C)C4)=N3)NC2=O)CC1 ; CC12CCN(CC1)c1cc(S(=O)(=O)CCO)ccc1C(=O)Nc1ccc(-n3cc(CN4CCCC(O)C4)cn3)c(n1)NC/C=C/CO2 ; CCS(=O)(=O)c1ccc2c(c1)N1CCC(C)(CC/C=C/CNc3nc(ccc3-n3cc(CN4CCCC(O)C4)cn3)NC2=O)CC1]

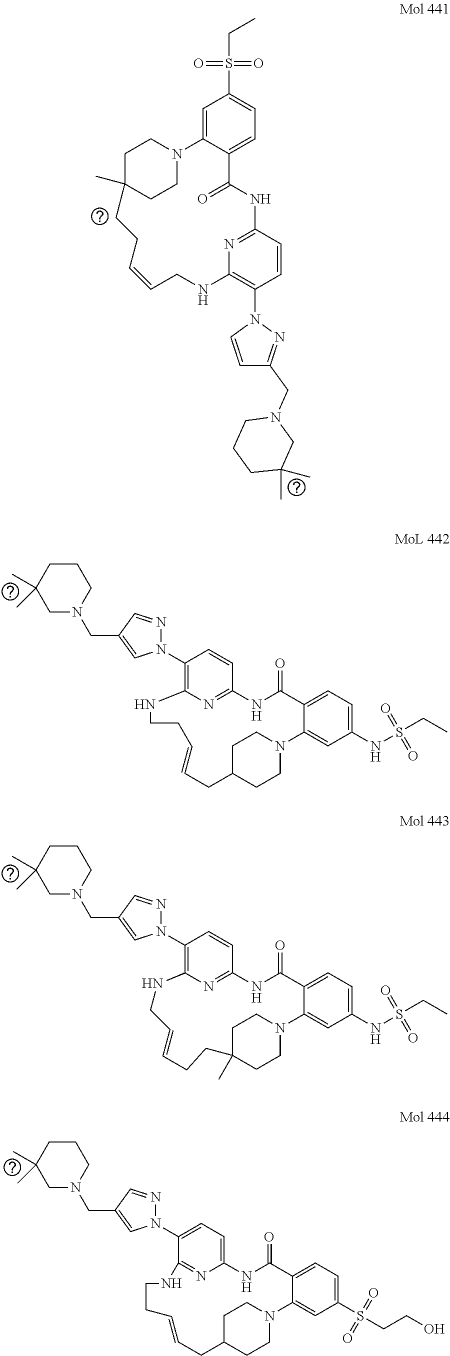

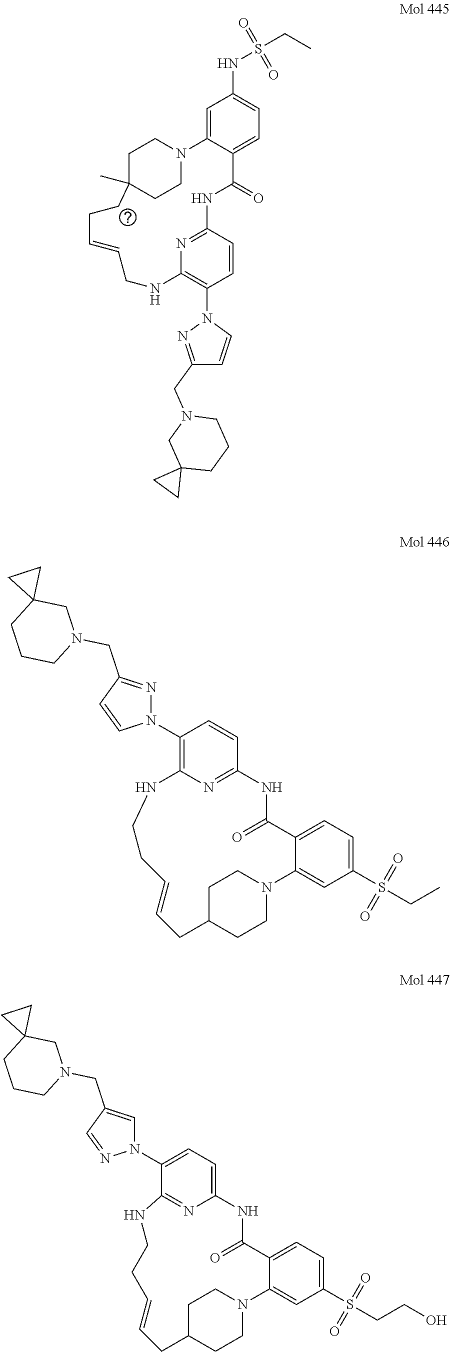 [SMILES: CCS(=O)(=O)Nc1ccc2c(c1)N1CCC(C)(CC/C=C/CNc3nc(ccc3-n3ccc(CN4CCCC5(CC5)C4)n3)NC2=O)CC1 ; CCS(=O)(=O)c1ccc2c(c1)N1CCC(C/C=C/CCNc3nc(ccc3-n3ccc(CN4CCCC5(CC5)C4)n3)NC2=O)CC1 ; O=C1Nc2ccc(-n3cc(CN4CCCC5(CC5)C4)cn3)c(n2)NCC/C=C/CC2CCN(CC2)c2cc(S(=O)(=O)CCO)ccc21]

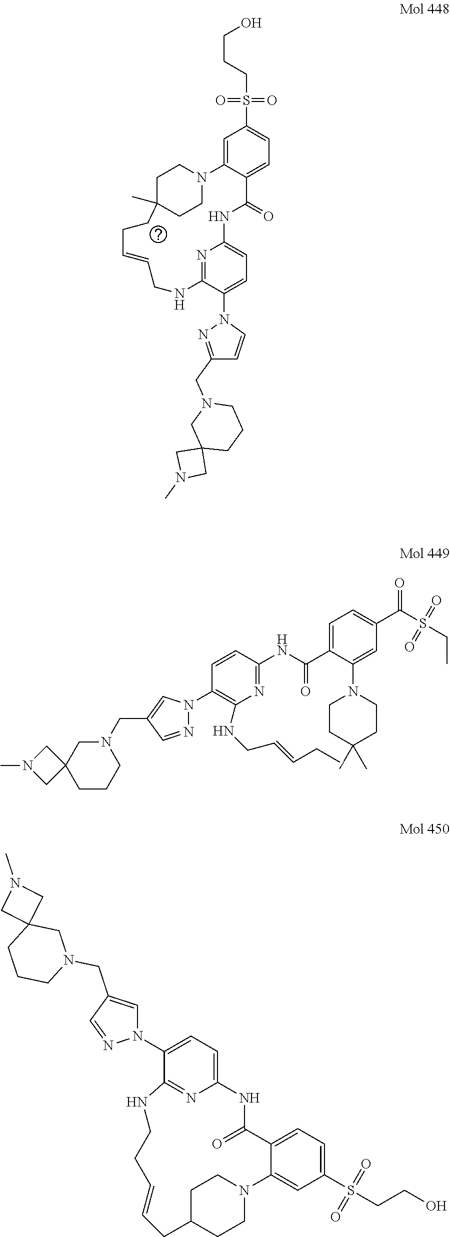 [SMILES: CN1CC2(CCCN(Cc3ccn(-c4ccc5nc4NC/C=C/CCC4(C)CCN(CC4)c4cc(S(=O)(=O)CCCO)ccc4C(=O)N5)n3)C2)C1 ; CC/C=C/CNc1nc(NC(=O)c2ccc(C(=O)S(=O)(=O)CC)cc2N2CCC(C)(C)CC2)ccc1-n1cc(CN2CCCC3(CN(C)C3)C2)cn1 ; CN1CC2(CCCN(Cc3cnn(-c4ccc5nc4NCC/C=C/CC4CCN(CC4)c4cc(S(=O)(=O)CCO)ccc4C(=O)N5)c3)C2)C1]

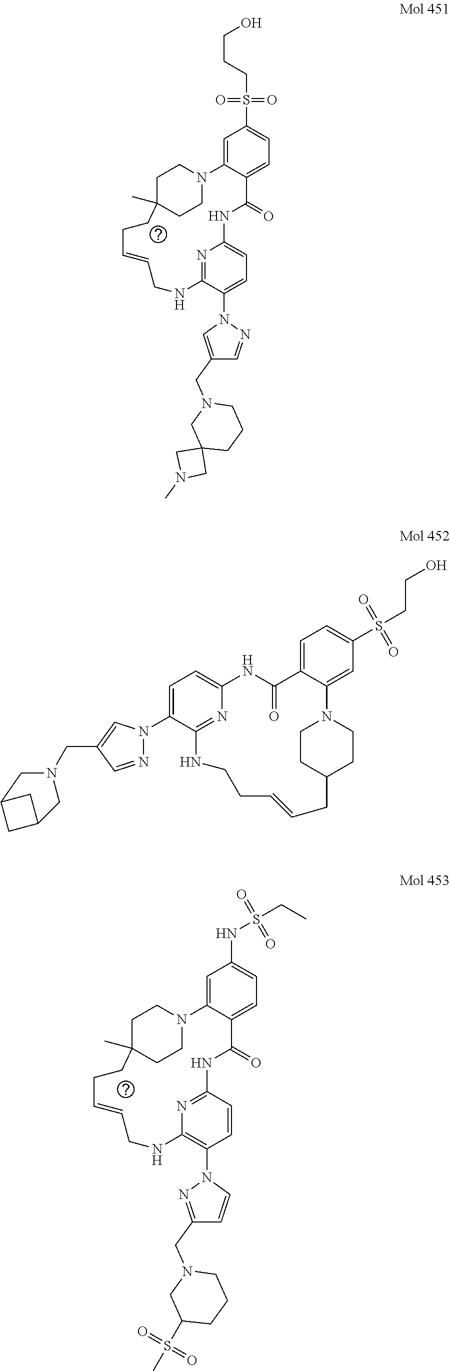 [SMILES: CN1CC2(CCCN(Cc3cnn(-c4ccc5nc4NC/C=C/CCC4(C)CCN(CC4)c4cc(S(=O)(=O)CCCO)ccc4C(=O)N5)c3)C2)C1 ; O=C1Nc2ccc(-n3cc(CN4CC5CC(C5)C4)cn3)c(n2)NCC/C=C/CC2CCN(CC2)c2cc(S(=O)(=O)CCO)ccc21 ; CCS(=O)(=O)Nc1ccc2c(c1)N1CCC(C)(CC/C=C/CNc3nc(ccc3-n3ccc(CN4CCCC(S(C)(=O)=O)C4)n3)NC2=O)CC1]

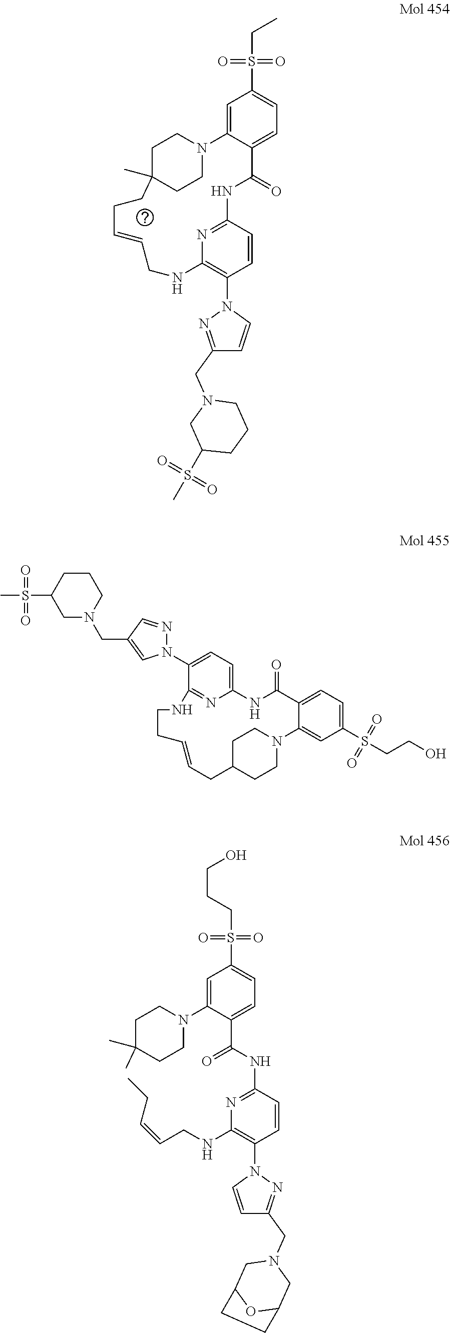 [SMILES: CCS(=O)(=O)c1ccc2c(c1)N1CCC(C)(CC/C=C/CNc3nc(ccc3-n3ccc(CN4CCCC(S(C)(=O)=O)C4)n3)NC2=O)CC1 ; CS(=O)(=O)C1CCCN(Cc2cnn(-c3ccc4nc3NCC/C=C/CC3CCN(CC3)c3cc(S(=O)(=O)CCO)ccc3C(=O)N4)c2)C1 ; CC/C=C\CNc1nc(NC(=O)c2ccc(S(=O)(=O)CCCO)cc2N2CCC(C)(C)CC2)ccc1-n1ccc(CN2CC3CCC(C2)O3)n1]

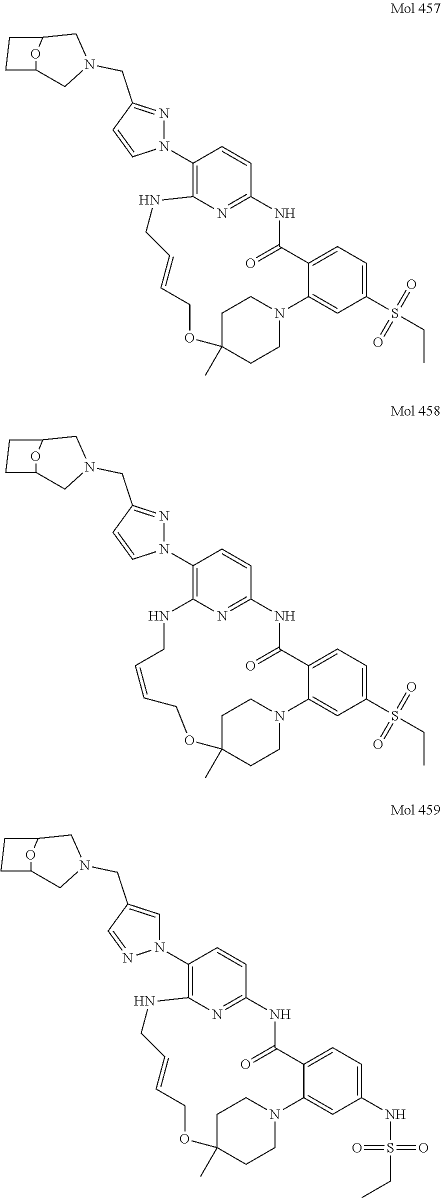 [SMILES: CCS(=O)(=O)c1ccc2c(c1)N1CCC(C)(CC1)OC/C=C/CNc1nc(ccc1-n1ccc(CN3CC4CCC(C3)O4)n1)NC2=O ; CCS(=O)(=O)c1ccc2c(c1)N1CCC(C)(CC1)OC/C=C\CNc1nc(ccc1-n1ccc(CN3CC4CCC(C3)O4)n1)NC2=O ; CCS(=O)(=O)Nc1ccc2c(c1)N1CCC(C)(CC1)OC/C=C/CNc1nc(ccc1-n1cc(CN3CC4CCC(C3)O4)cn1)NC2=O]

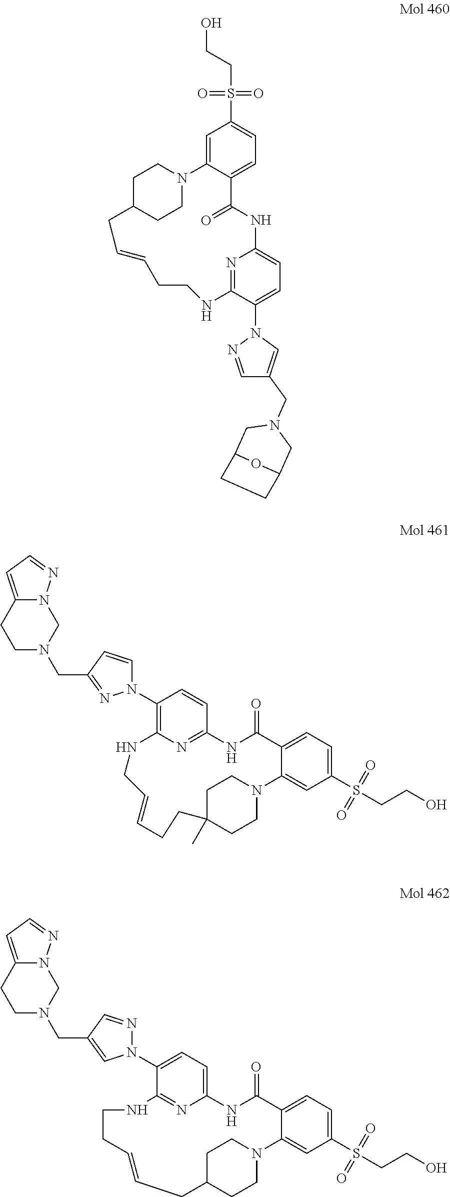 [SMILES: O=C1Nc2ccc(-n3cc(CN4CC5CCC(C4)O5)cn3)c(n2)NCC/C=C/CC2CCN(CC2)c2cc(S(=O)(=O)CCO)ccc21 ; CC12CC/C=C/CNc3nc(ccc3-n3ccc(CN4CCc5ccnn5C4)n3)NC(=O)c3ccc(S(=O)(=O)CCO)cc3N(CC1)CC2 ; O=C1Nc2ccc(-n3cc(CN4CCc5ccnn5C4)cn3)c(n2)NCC/C=C/CC2CCN(CC2)c2cc(S(=O)(=O)CCO)ccc21]

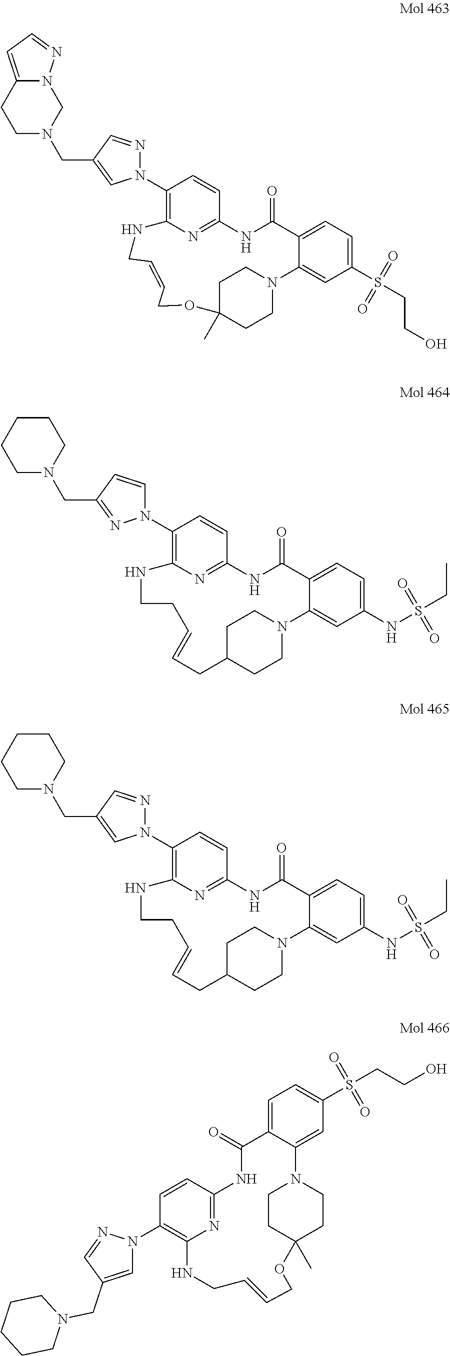 [SMILES: CC12CCN(CC1)c1cc(S(=O)(=O)CCO)ccc1C(=O)Nc1ccc(-n3cc(CN4CCc5ccnn5C4)cn3)c(n1)NC/C=C\CO2 ; CCS(=O)(=O)Nc1ccc2c(c1)N1CCC(C/C=C/CCNc3nc(ccc3-n3ccc(CN4CCCCC4)n3)NC2=O)CC1 ; CCS(=O)(=O)Nc1ccc2c(c1)N1CCC(C/C=C/CCNc3nc(ccc3-n3cc(CN4CCCCC4)cn3)NC2=O)CC1 ; CC12CCN(CC1)c1cc(S(=O)(=O)CCO)ccc1C(=O)Nc1ccc(-n3cc(CN4CCCCC4)cn3)c(n1)NC/C=C/CO2]

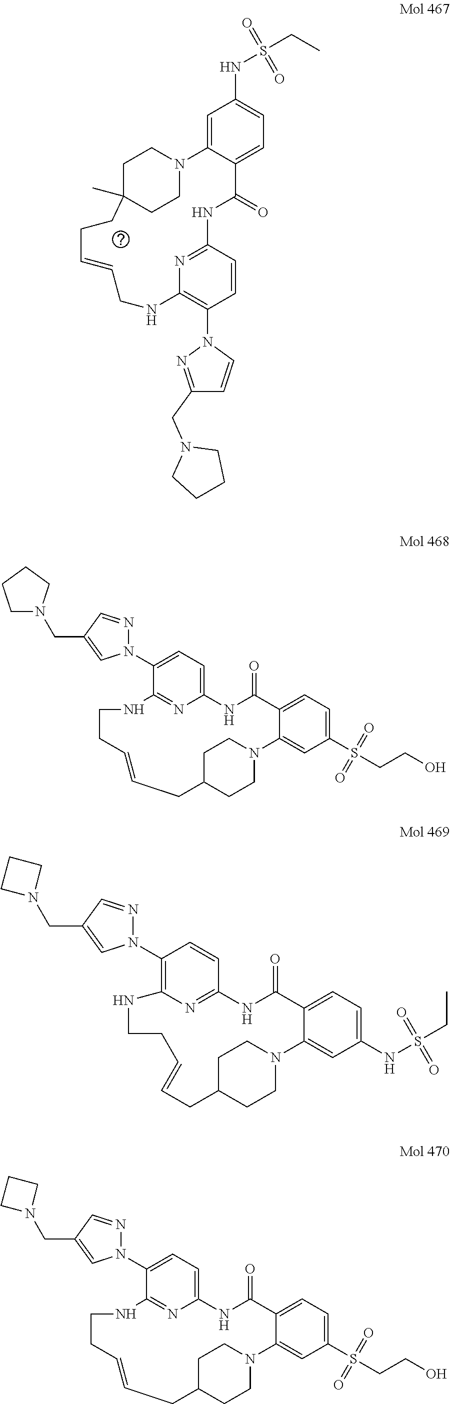 [SMILES: CCS(=O)(=O)Nc1ccc2c(c1)N1CCC(C)(CC/C=C/CNc3nc(ccc3-n3ccc(CN4CCCC4)n3)NC2=O)CC1 ; O=C1Nc2ccc(-n3cc(CN4CCCC4)cn3)c(n2)NCC/C=C/CC2CCN(CC2)c2cc(S(=O)(=O)CCO)ccc21 ; CCS(=O)(=O)Nc1ccc2c(c1)N1CCC(C/C=C/CCNc3nc(ccc3-n3cc(CN4CCC4)cn3)NC2=O)CC1 ; O=C1Nc2ccc(-n3cc(CN4CCC4)cn3)c(n2)NCC/C=C/CC2CCN(CC2)c2cc(S(=O)(=O)CCO)ccc21]

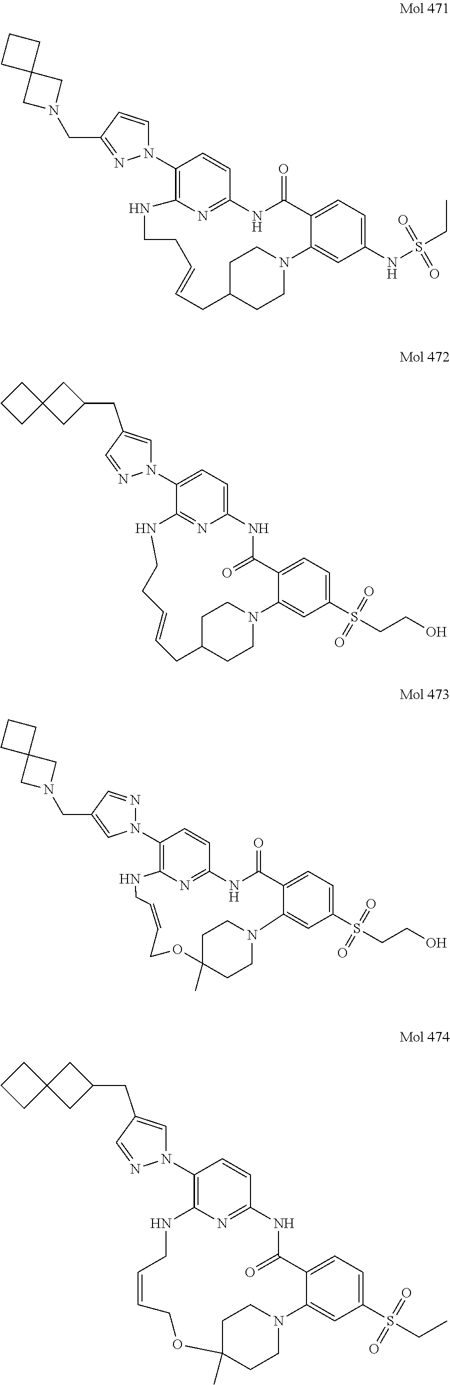 [SMILES: CCS(=O)(=O)Nc1ccc2c(c1)N1CCC(C/C=C/CCNc3nc(ccc3-n3ccc(CN4CC5(CCC5)C4)n3)NC2=O)CC1 ; O=C1Nc2ccc(-n3cc(CC4CC5(CCC5)C4)cn3)c(n2)NCC/C=C/CC2CCN(CC2)c2cc(S(=O)(=O)CCO)ccc21 ; CC12CCN(CC1)c1cc(S(=O)(=O)CCO)ccc1C(=O)Nc1ccc(-n3cc(CN4CC5(CCC5)C4)cn3)c(n1)NC/C=C\CO2 ; CCS(=O)(=O)c1ccc2c(c1)N1CCC(C)(CC1)OC/C=C\CNc1nc(ccc1-n1cc(CC3CC4(CCC4)C3)cn1)NC2=O]

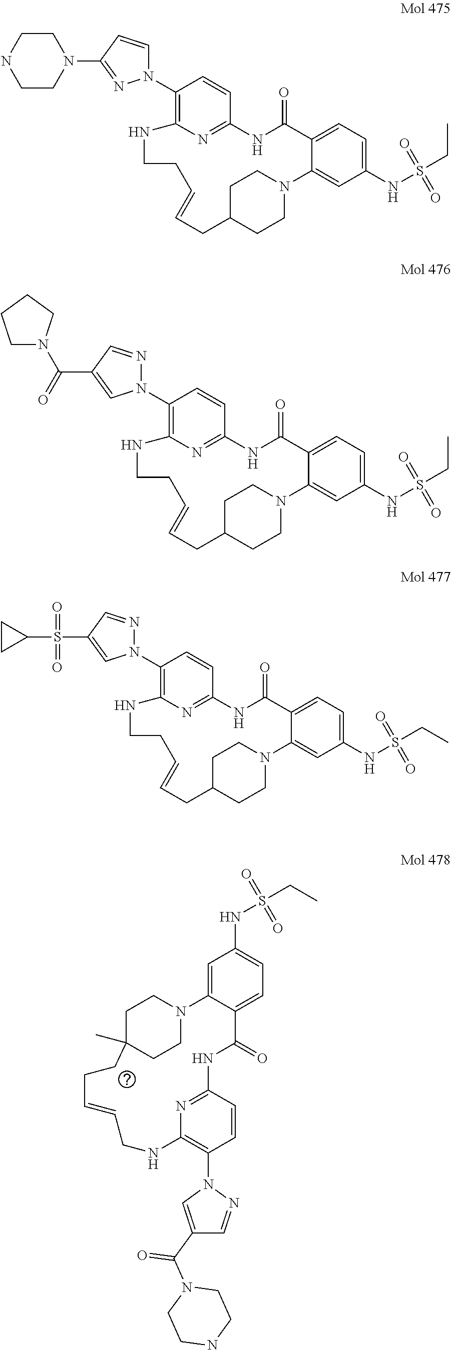 [SMILES: CCS(=O)(=O)Nc1ccc2c(c1)N1CCC(C/C=C/CCNc3nc(ccc3-n3ccc(N4CCNCC4)n3)NC2=O)CC1 ; CCS(=O)(=O)Nc1ccc2c(c1)N1CCC(C/C=C/CCNc3nc(ccc3-n3cc(C(=O)N4CCCC4)cn3)NC2=O)CC1 ; CCS(=O)(=O)Nc1ccc2c(c1)N1CCC(C/C=C/CCNc3nc(ccc3-n3cc(S(=O)(=O)C4CC4)cn3)NC2=O)CC1 ; CCS(=O)(=O)Nc1ccc2c(c1)N1CCC(C)(CC/C=C/CNc3nc(ccc3-n3cc(C(=O)N4CCNCC4)cn3)NC2=O)CC1]

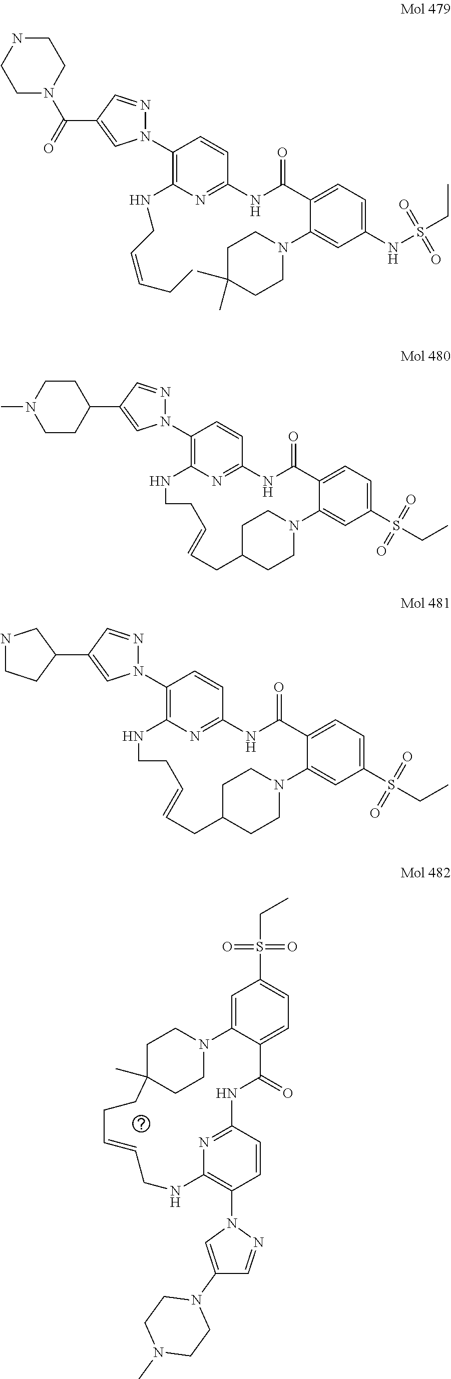

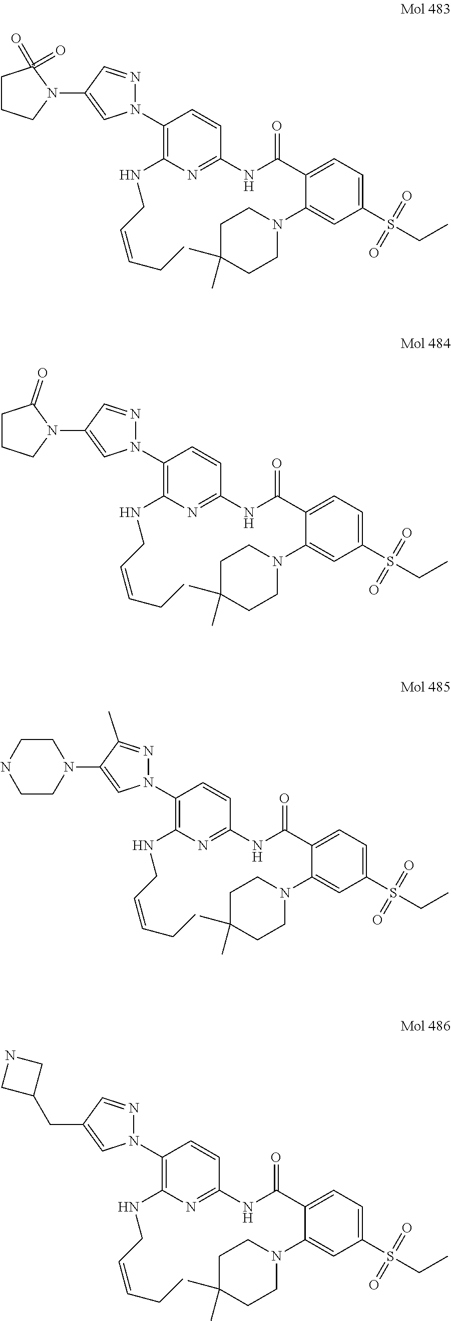

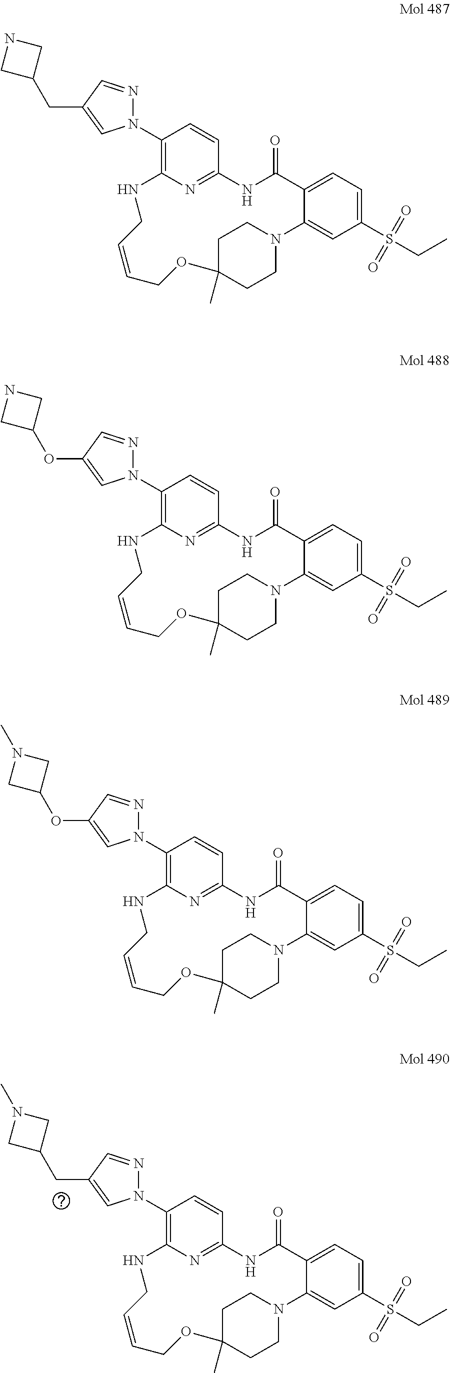

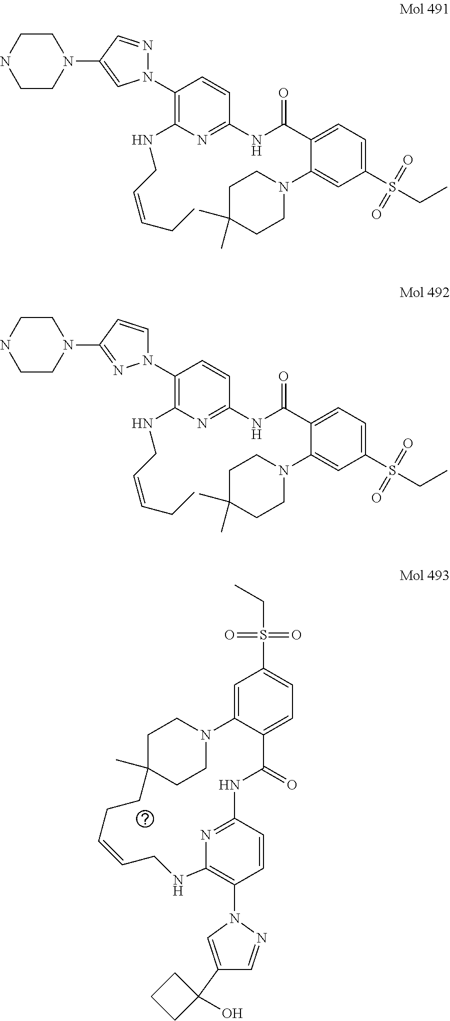 [SMILES: CC/C=C\CNc1nc(NC(=O)c2ccc(S(=O)(=O)CC)cc2N2CCC(C)(C)CC2)ccc1-n1cc(N2CCNCC2)cn1 ; CC/C=C\CNc1nc(NC(=O)c2ccc(S(=O)(=O)CC)cc2N2CCC(C)(C)CC2)ccc1-n1ccc(N2CCNCC2)n1 ; CCS(=O)(=O)c1ccc2c(c1)N1CCC(C)(CC/C=C\CNc3nc(ccc3-n3cc(C4(O)CCC4)cn3)NC2=O)CC1]

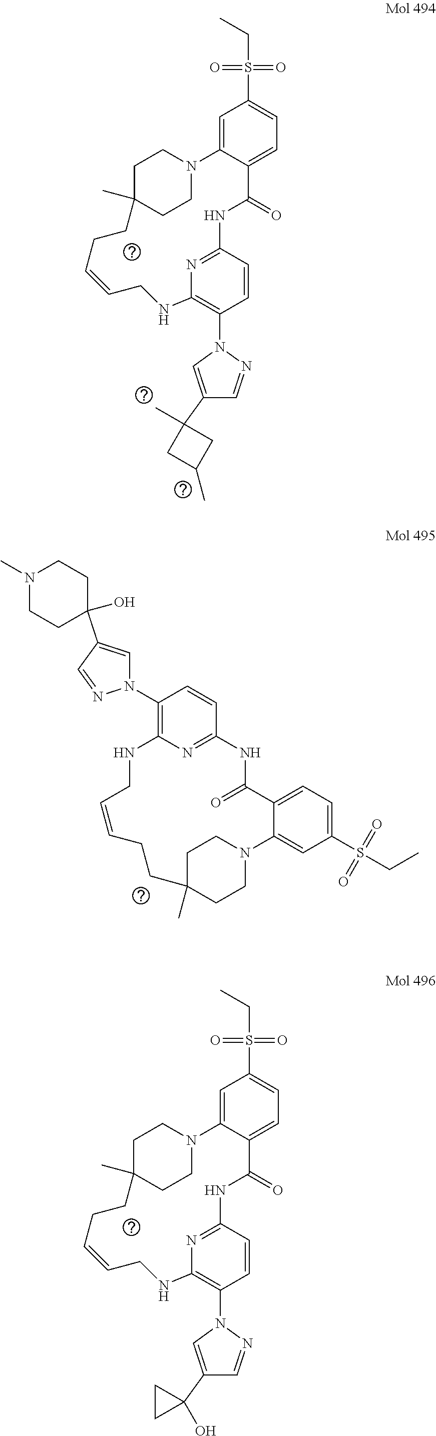 [SMILES: CCS(=O)(=O)c1ccc2c(c1)N1CCC(C)(CC/C=C\CNc3nc(ccc3-n3cc(C4(C)CC(C)C4)cn3)NC2=O)CC1 ; CCS(=O)(=O)c1ccc2c(c1)N1CCC(C)(CC/C=C\CNc3nc(ccc3-n3cc(C4(O)CCN(C)CC4)cn3)NC2=O)CC1 ; CCS(=O)(=O)c1ccc2c(c1)N1CCC(C)(CC/C=C\CNc3nc(ccc3-n3cc(C4(O)CC4)cn3)NC2=O)CC1]

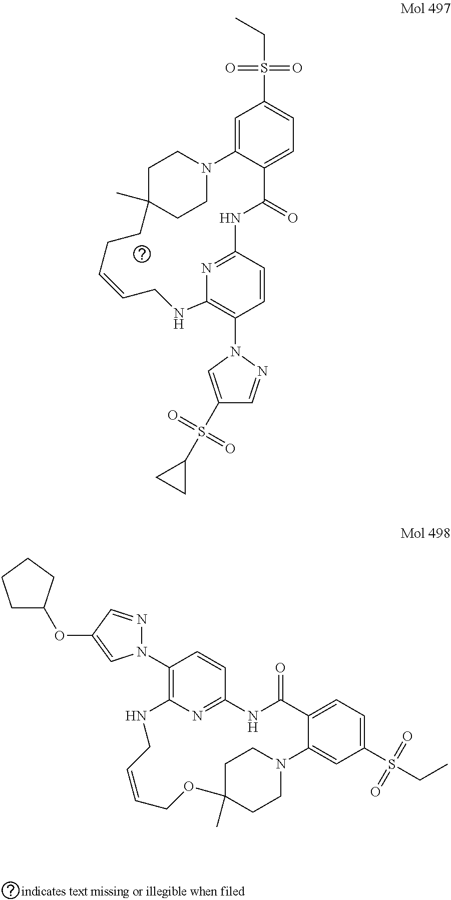 [SMILES: CCS(=O)(=O)c1ccc2c(c1)N1CCC(C)(CC/C=C\CNc3nc(ccc3-n3cc(S(=O)(=O)C4CC4)cn3)NC2=O)CC1 ; CCS(=O)(=O)c1ccc2c(c1)N1CCC(C)(CC1)OC/C=C\CNc1nc(ccc1-n1cc(OC3CCCC3)cn1)NC2=O]

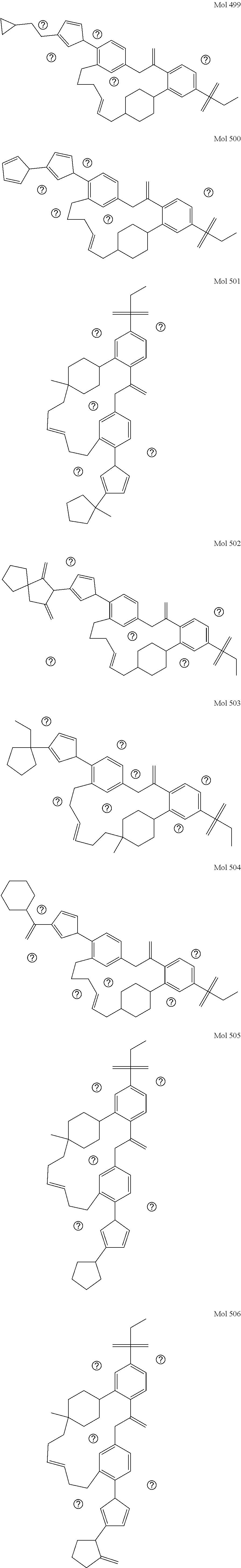

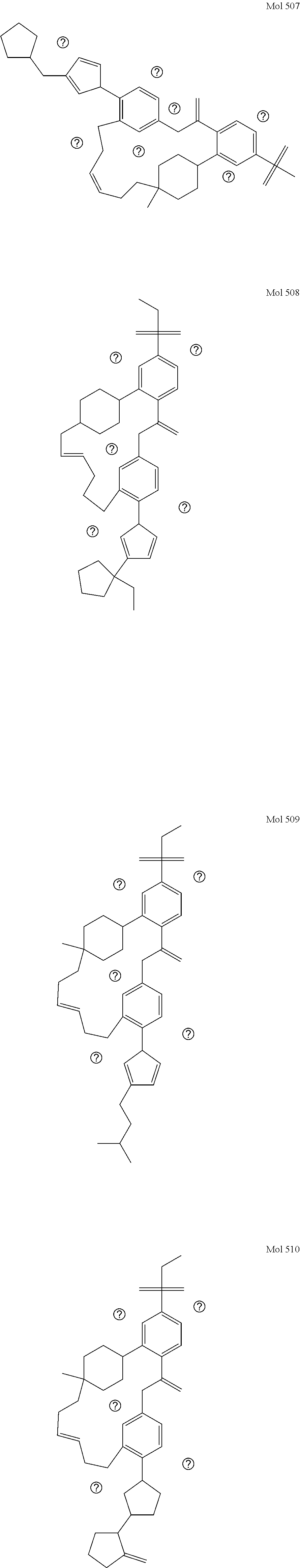

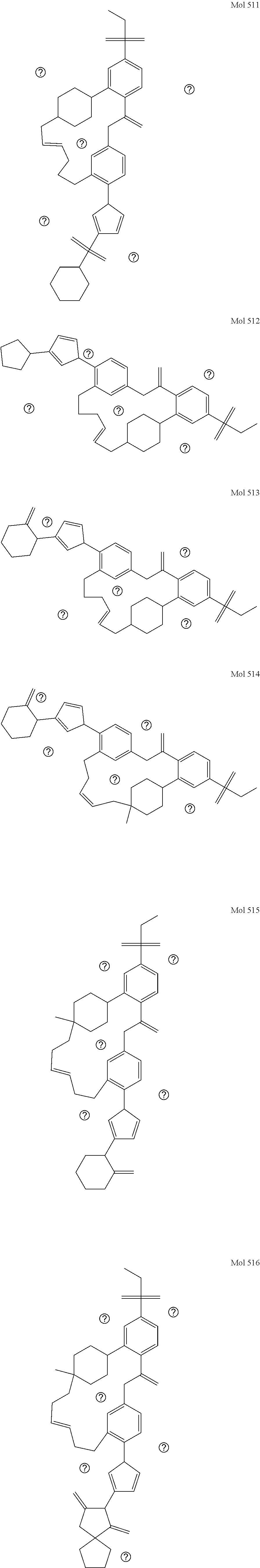

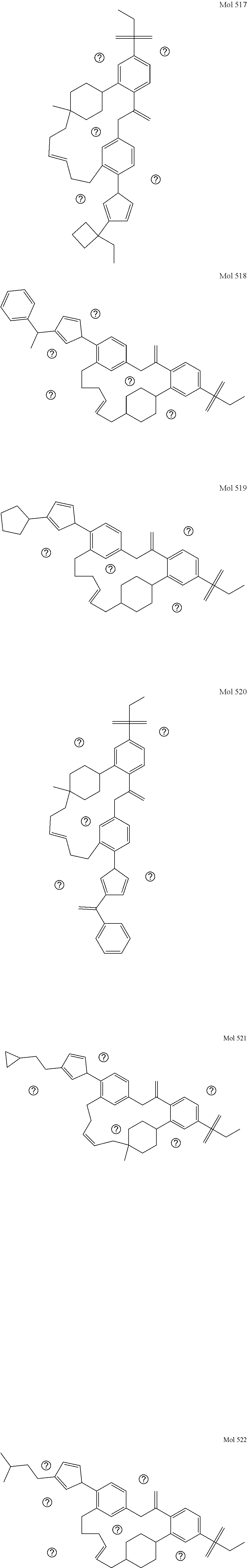

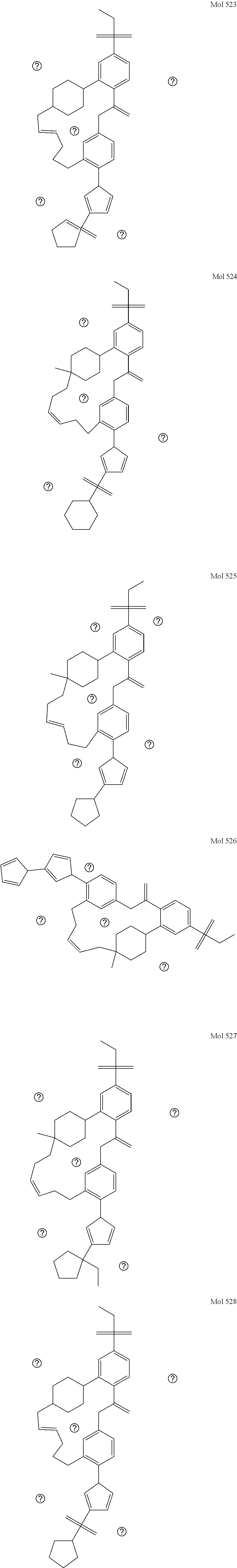

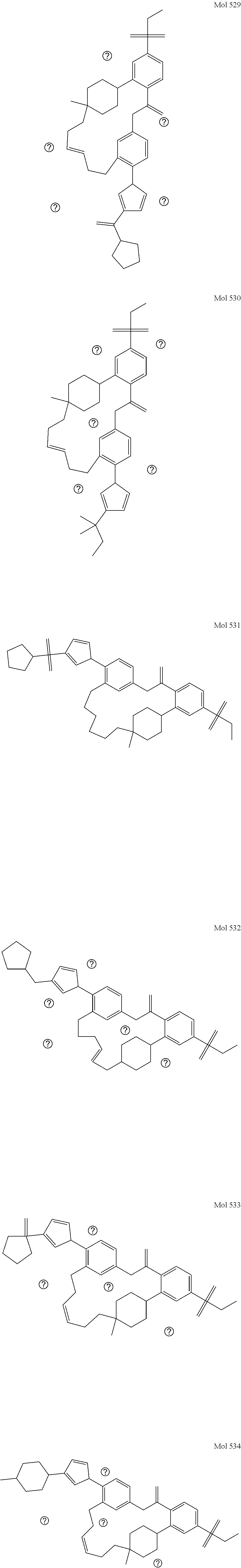

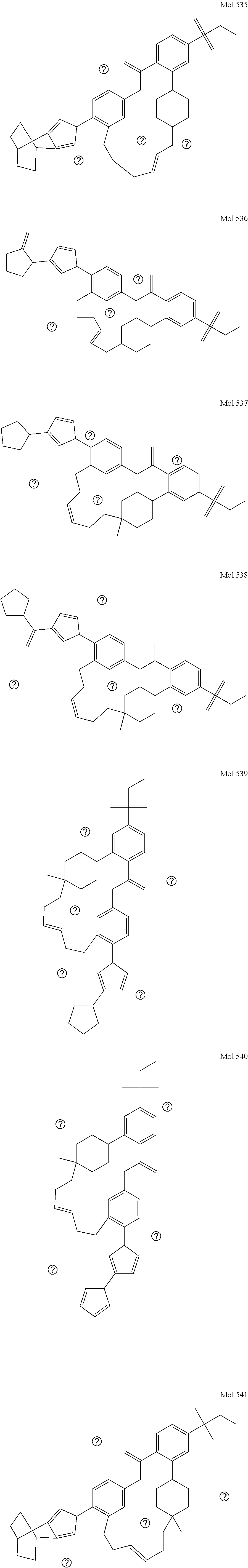

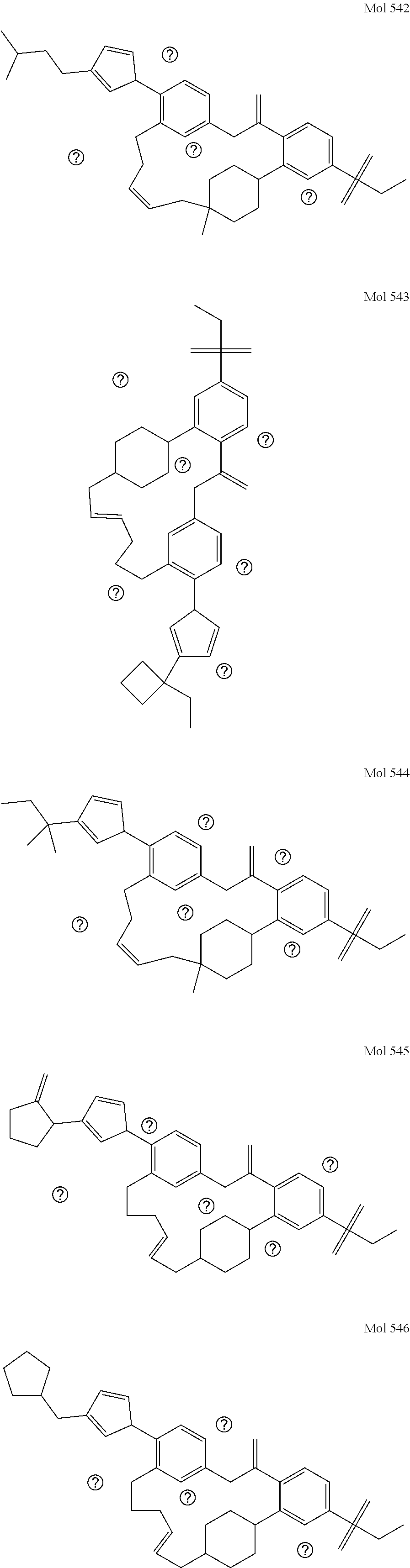

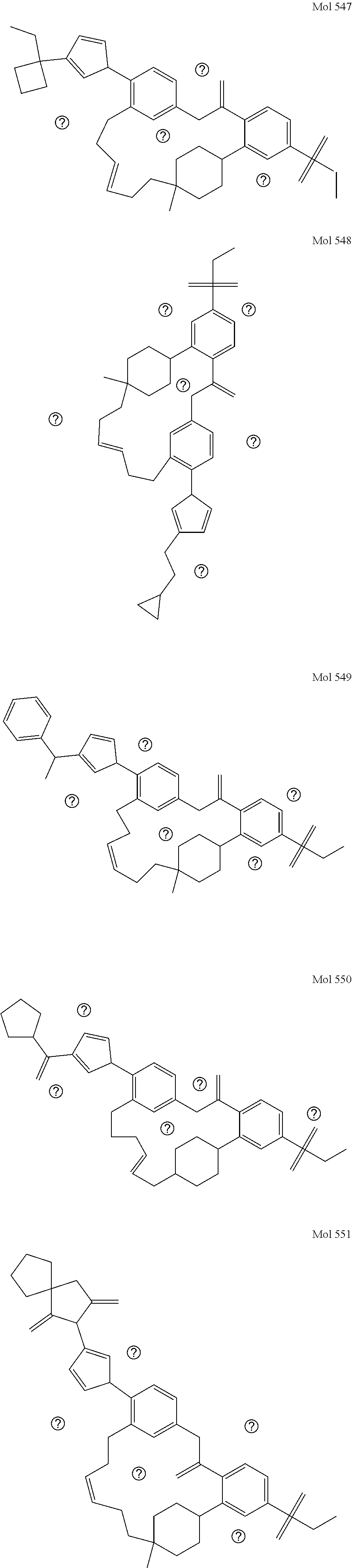

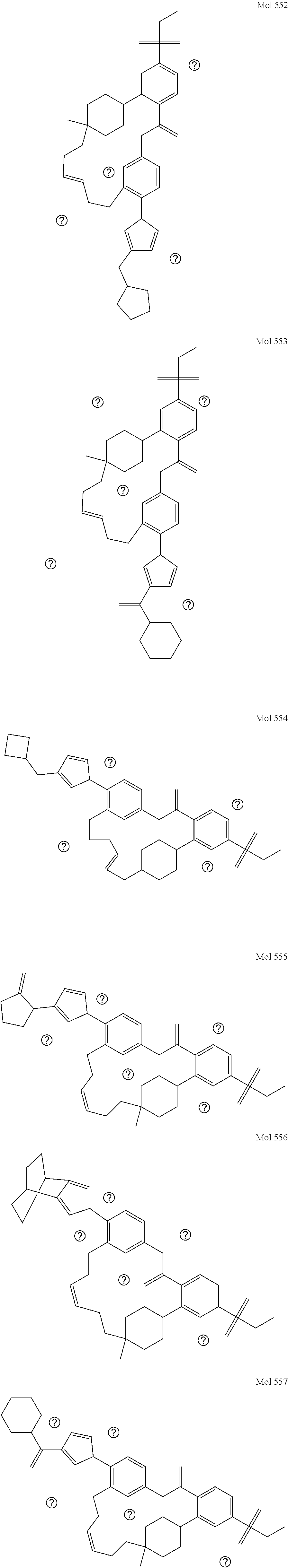

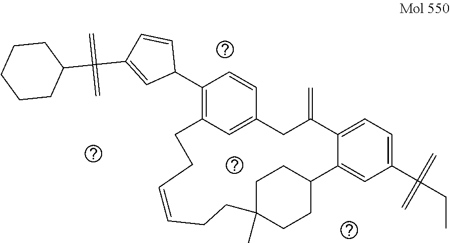

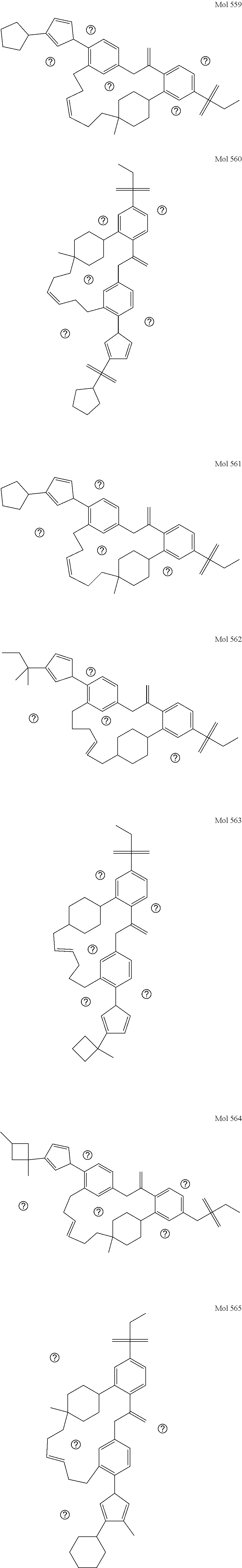

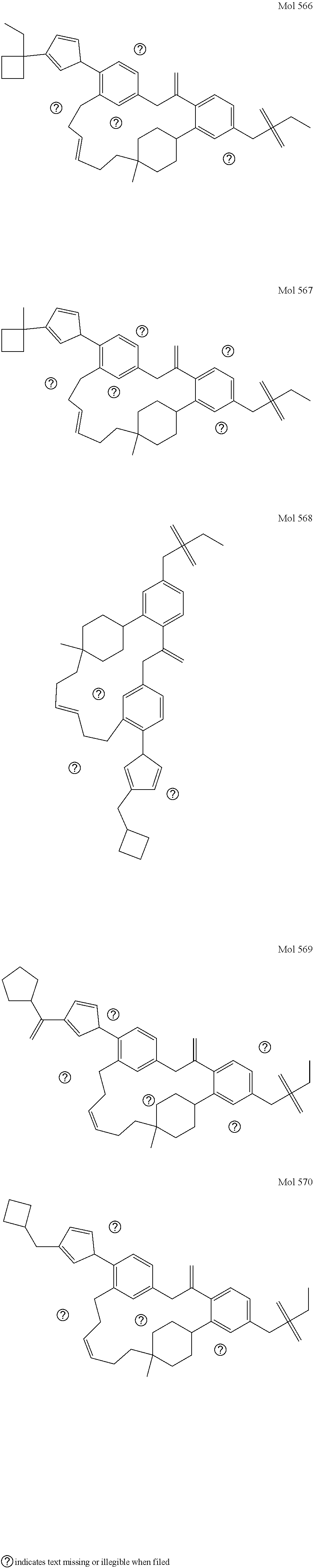

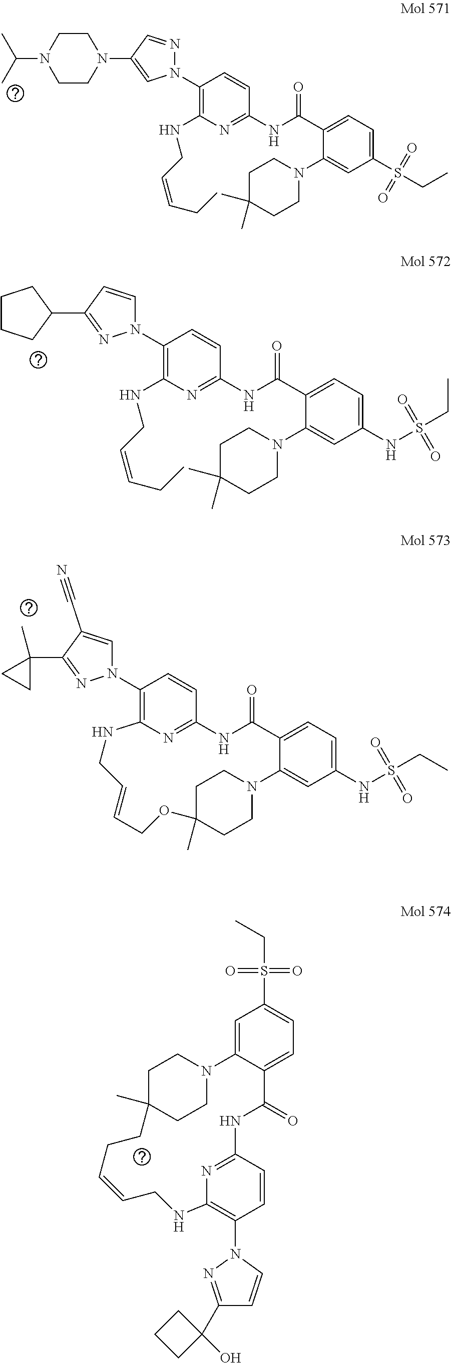 [SMILES: CC/C=C\CNc1nc(NC(=O)c2ccc(S(=O)(=O)CC)cc2N2CCC(C)(C)CC2)ccc1-n1cc(N2CCN(C(C)C)CC2)cn1 ; CC/C=C\CNc1nc(NC(=O)c2ccc(NS(=O)(=O)CC)cc2N2CCC(C)(C)CC2)ccc1-n1ccc(C2CCCC2)n1 ; CCS(=O)(=O)Nc1ccc2c(c1)N1CCC(C)(CC1)OC/C=C/CNc1nc(ccc1-n1cc(C#N)c(C3(C)CC3)n1)NC2=O ; CCS(=O)(=O)c1ccc2c(c1)N1CCC(C)(CC/C=C\CNc3nc(ccc3-n3ccc(C4(O)CCC4)n3)NC2=O)CC1]

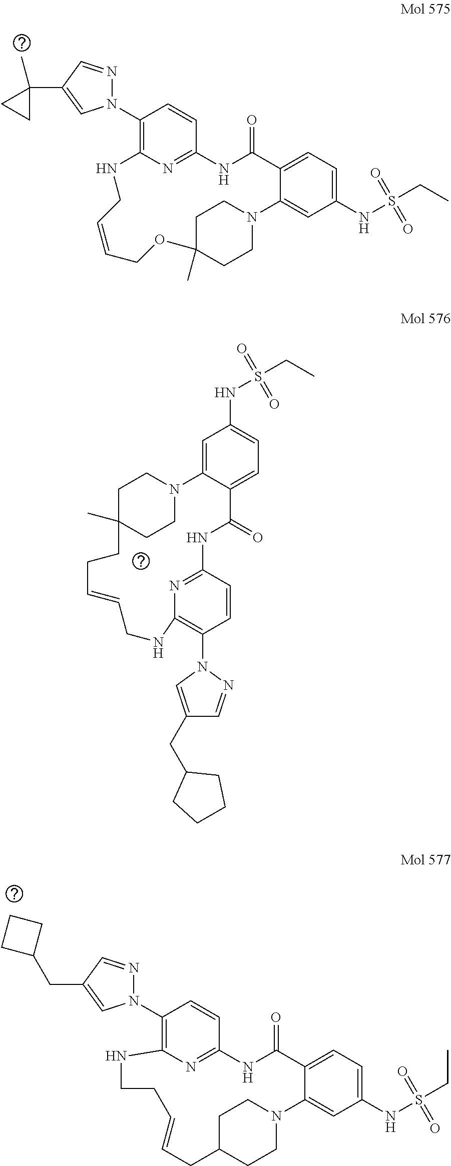

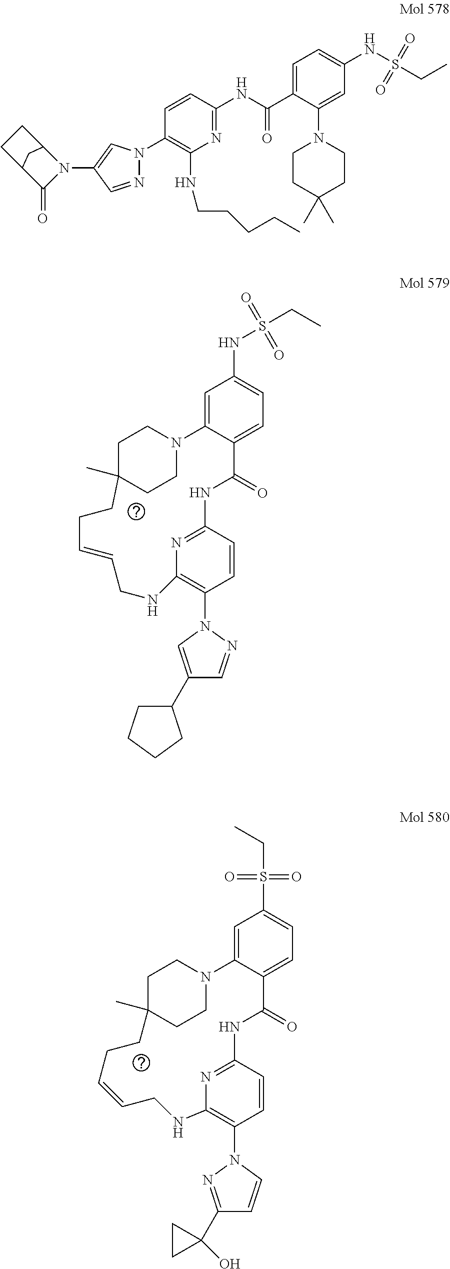 [SMILES: CCCCCNc1nc(NC(=O)c2ccc(NS(=O)(=O)CC)cc2N2CCC(C)(C)CC2)ccc1-n1cc(N2C(=O)C3CCC2C3)cn1 ; CCS(=O)(=O)Nc1ccc2c(c1)N1CCC(C)(CC/C=C/CNc3nc(ccc3-n3cc(C4CCCC4)cn3)NC2=O)CC1 ; CCS(=O)(=O)c1ccc2c(c1)N1CCC(C)(CC/C=C\CNc3nc(ccc3-n3ccc(C4(O)CC4)n3)NC2=O)CC1]

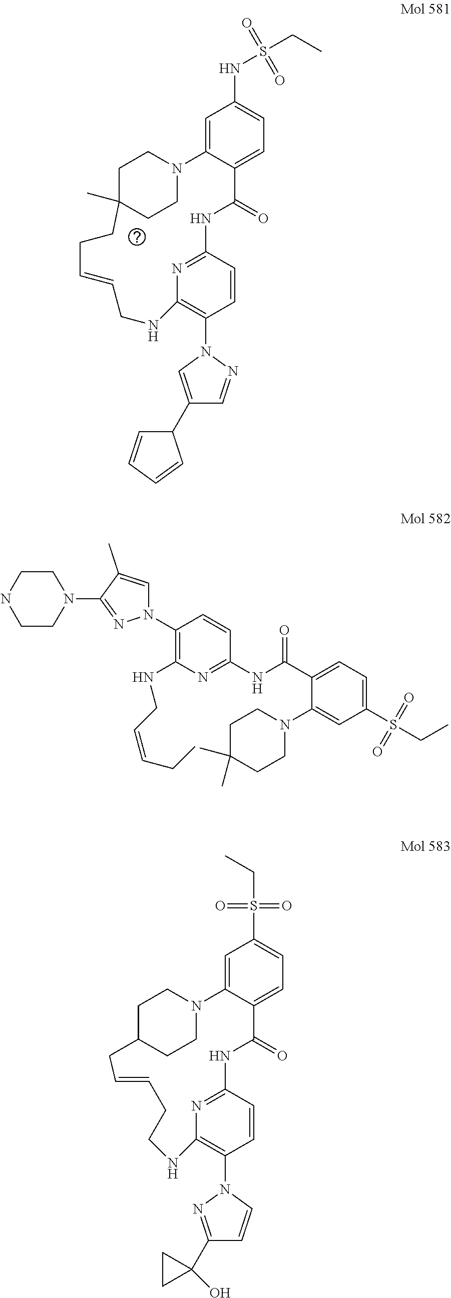 [SMILES: CCS(=O)(=O)Nc1ccc2c(c1)N1CCC(C)(CC/C=C/CNc3nc(ccc3-n3cc(C4C=CC=C4)cn3)NC2=O)CC1 ; CC/C=C\CNc1nc(NC(=O)c2ccc(S(=O)(=O)CC)cc2N2CCC(C)(C)CC2)ccc1-n1cc(C)c(N2CCNCC2)n1 ; CCS(=O)(=O)c1ccc2c(c1)N1CCC(C/C=C/CCNc3nc(ccc3-n3ccc(C4(O)CC4)n3)NC2=O)CC1]

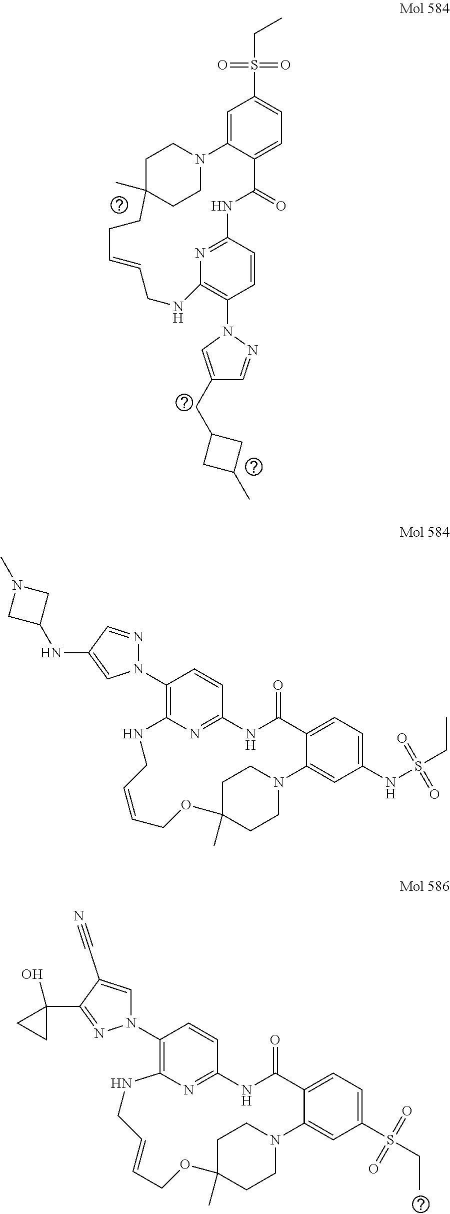 [SMILES: CCS(=O)(=O)c1ccc2c(c1)N1CCC(C)(CC/C=C/CNc3nc(ccc3-n3cc(CC4CC(C)C4)cn3)NC2=O)CC1 ; CCS(=O)(=O)Nc1ccc2c(c1)N1CCC(C)(CC1)OC/C=C\CNc1nc(ccc1-n1cc(NC3CN(C)C3)cn1)NC2=O ; CCS(=O)(=O)c1ccc2c(c1)N1CCC(C)(CC1)OC/C=C/CNc1nc(ccc1-n1cc(C#N)c(C3(O)CC3)n1)NC2=O]

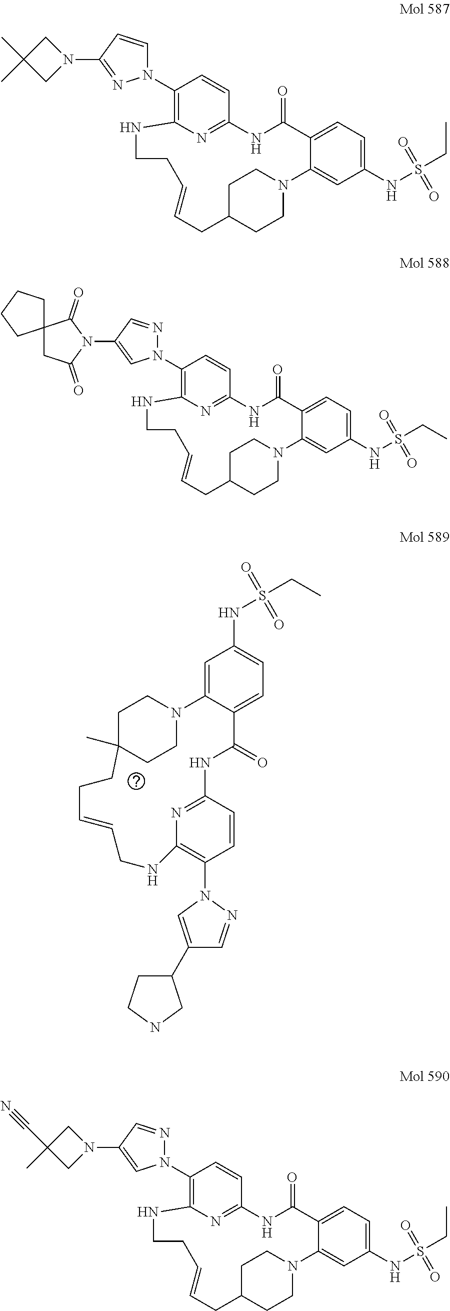 [SMILES: CCS(=O)(=O)Nc1ccc2c(c1)N1CCC(C/C=C/CCNc3nc(ccc3-n3ccc(N4CC(C)(C)C4)n3)NC2=O)CC1 ; CCS(=O)(=O)Nc1ccc2c(c1)N1CCC(C/C=C/CCNc3nc(ccc3-n3cc(N4C(=O)CC5(CCCC5)C4=O)cn3)NC2=O)CC1 ; CCS(=O)(=O)Nc1ccc2c(c1)N1CCC(C)(CC/C=C/CNc3nc(ccc3-n3cc(C4CCNC4)cn3)NC2=O)CC1 ; CCS(=O)(=O)Nc1ccc2c(c1)N1CCC(C/C=C/CCNc3nc(ccc3-n3cc(N4CC(C)(C#N)C4)cn3)NC2=O)CC1]

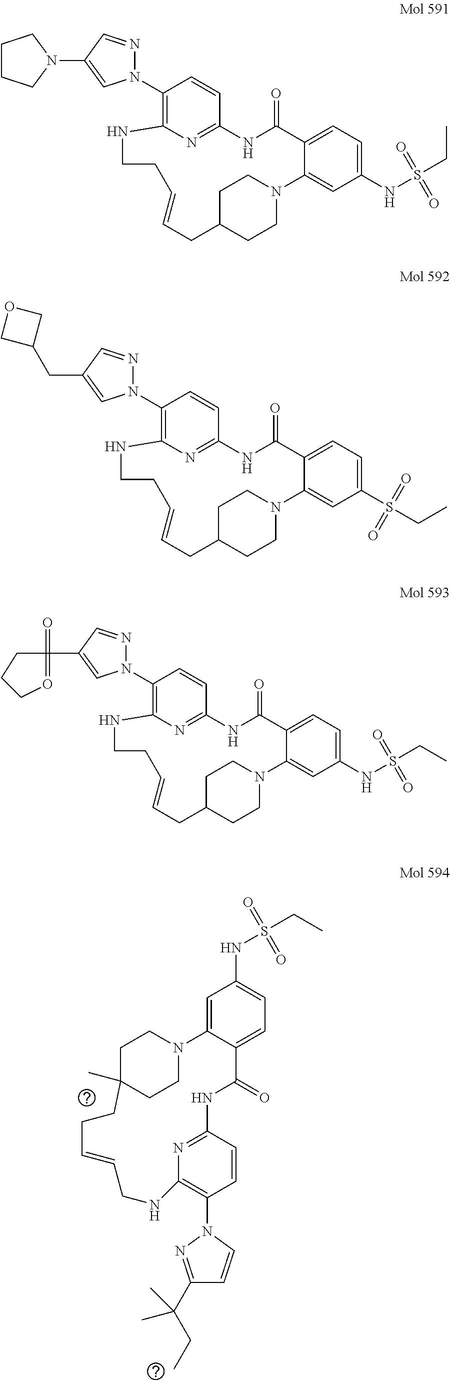

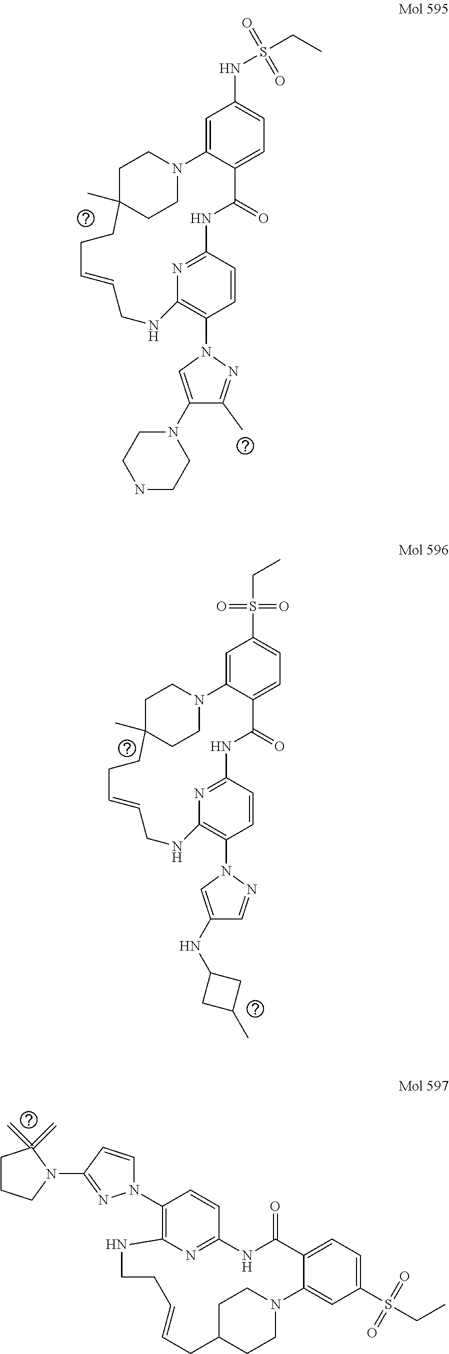

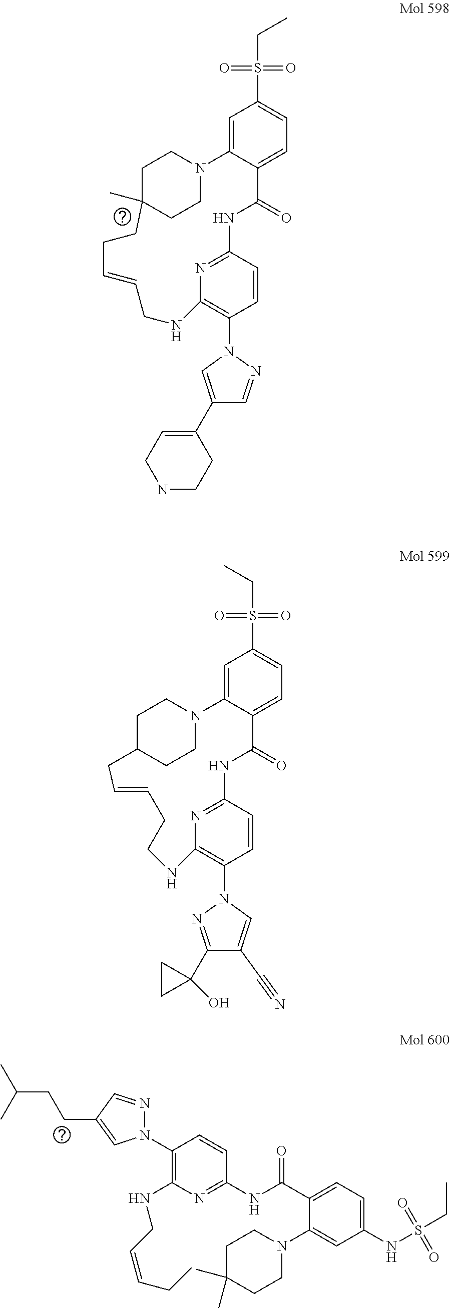 [SMILES: CCS(=O)(=O)c1ccc2c(c1)N1CCC(C)(CC/C=C/CNc3nc(ccc3-n3cc(C4=CCNCC4)cn3)NC2=O)CC1 ; CCS(=O)(=O)c1ccc2c(c1)N1CCC(C/C=C/CCNc3nc(ccc3-n3cc(C#N)c(C4(O)CC4)n3)NC2=O)CC1 ; CC/C=C\CNc1nc(NC(=O)c2ccc(NS(=O)(=O)CC)cc2N2CCC(C)(C)CC2)ccc1-n1cc(CCC(C)C)cn1]

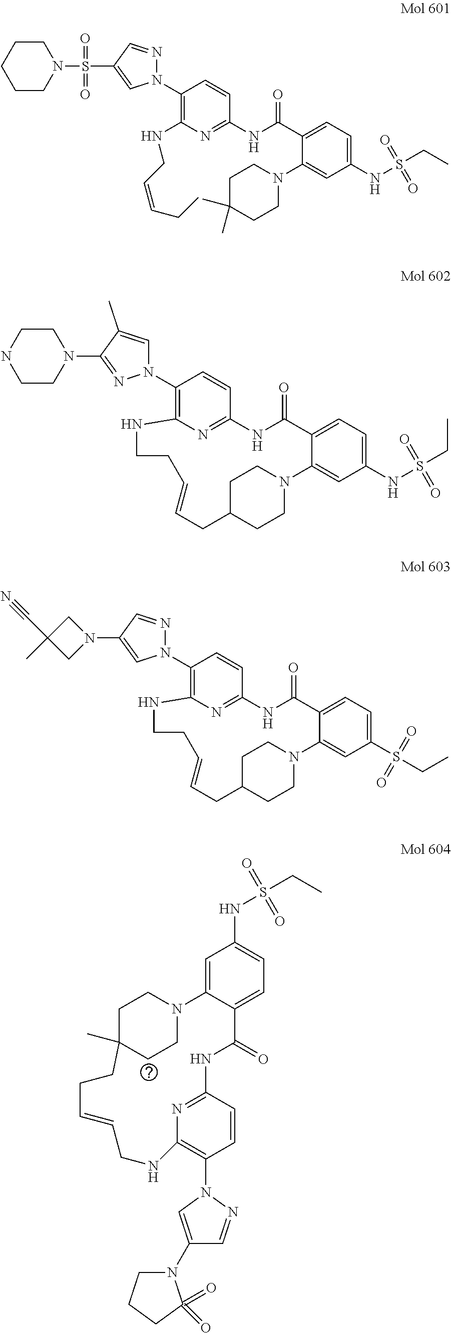

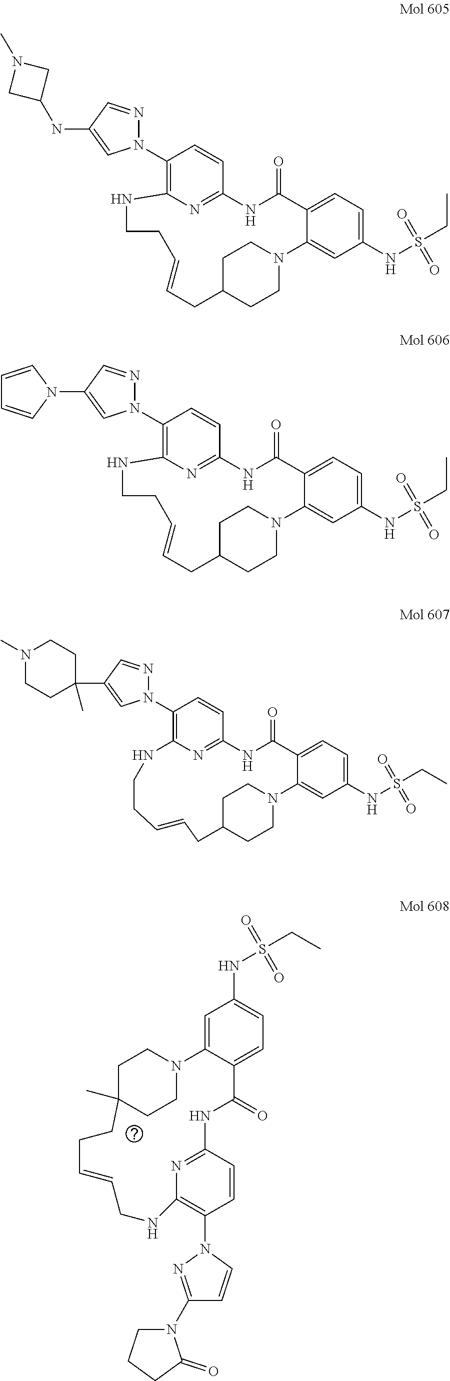

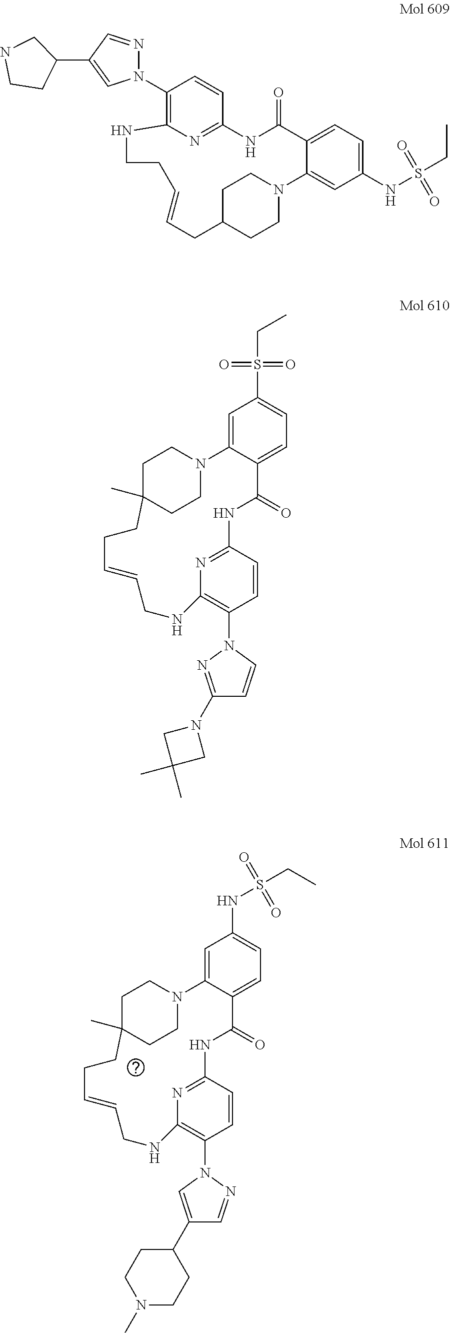 [SMILES: CCS(=O)(=O)Nc1ccc2c(c1)N1CCC(C/C=C/CCNc3nc(ccc3-n3cc(C4CCNC4)cn3)NC2=O)CC1 ; CCS(=O)(=O)c1ccc2c(c1)N1CCC(C)(CC/C=C/CNc3nc(ccc3-n3ccc(N4CC(C)(C)C4)n3)NC2=O)CC1 ; CCS(=O)(=O)Nc1ccc2c(c1)N1CCC(C)(CC/C=C/CNc3nc(ccc3-n3cc(C4CCN(C)CC4)cn3)NC2=O)CC1]

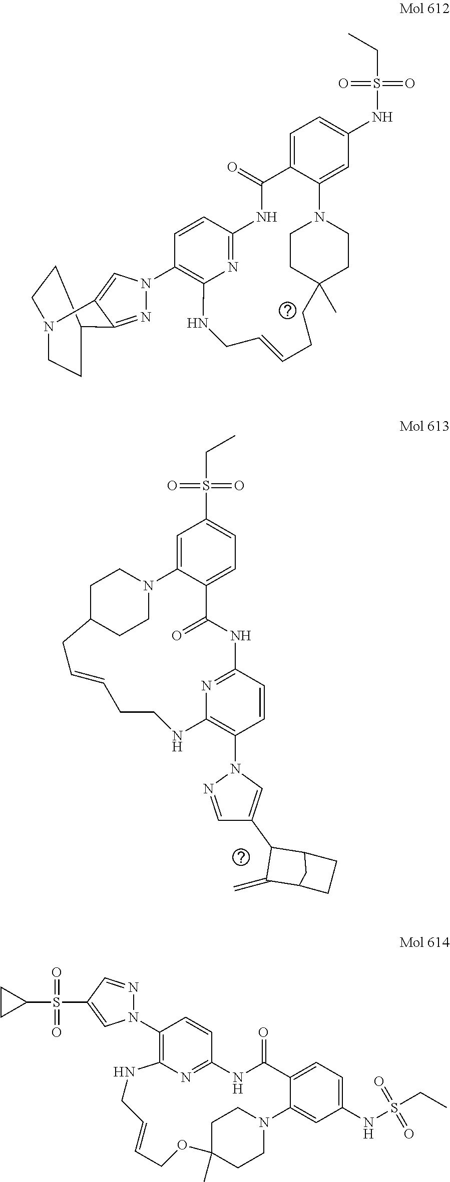 [SMILES: CCS(=O)(=O)Nc1ccc2c(c1)N1CCC(C)(CC/C=C/CNc3nc(ccc3-n3cc4c(n3)C3CCN4CC3)NC2=O)CC1 ; C=C1C2CCC(C2)C1c1cnn(-c2ccc3nc2NCC/C=C/CC2CCN(CC2)c2cc(S(=O)(=O)CC)ccc2C(=O)N3)c1 ; CCS(=O)(=O)Nc1ccc2c(c1)N1CCC(C)(CC1)OC/C=C/CNc1nc(ccc1-n1cc(S(=O)(=O)C3CC3)cn1)NC2=O]

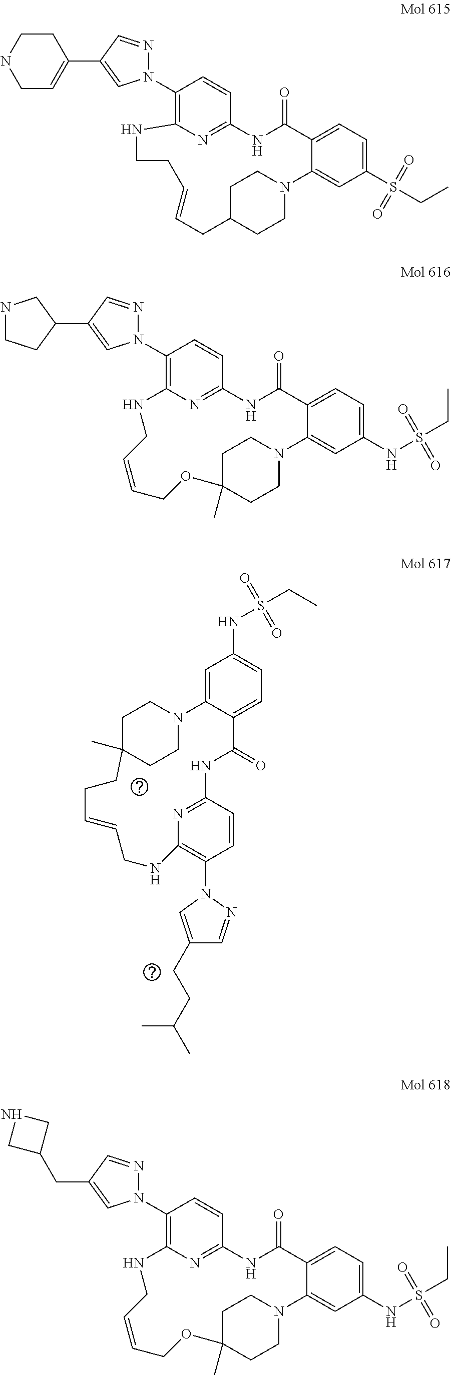 [SMILES: CCS(=O)(=O)c1ccc2c(c1)N1CCC(C/C=C/CCNc3nc(ccc3-n3cc(C4=CCNCC4)cn3)NC2=O)CC1 ; CCS(=O)(=O)Nc1ccc2c(c1)N1CCC(C)(CC1)OC/C=C\CNc1nc(ccc1-n1cc(C3CCNC3)cn1)NC2=O ; CCS(=O)(=O)Nc1ccc2c(c1)N1CCC(C)(CC/C=C/CNc3nc(ccc3-n3cc(CCC(C)C)cn3)NC2=O)CC1 ; CCS(=O)(=O)Nc1ccc2c(c1)N1CCC(C)(CC1)OC/C=C\CNc1nc(ccc1-n1cc(CC3CNC3)cn1)NC2=O]

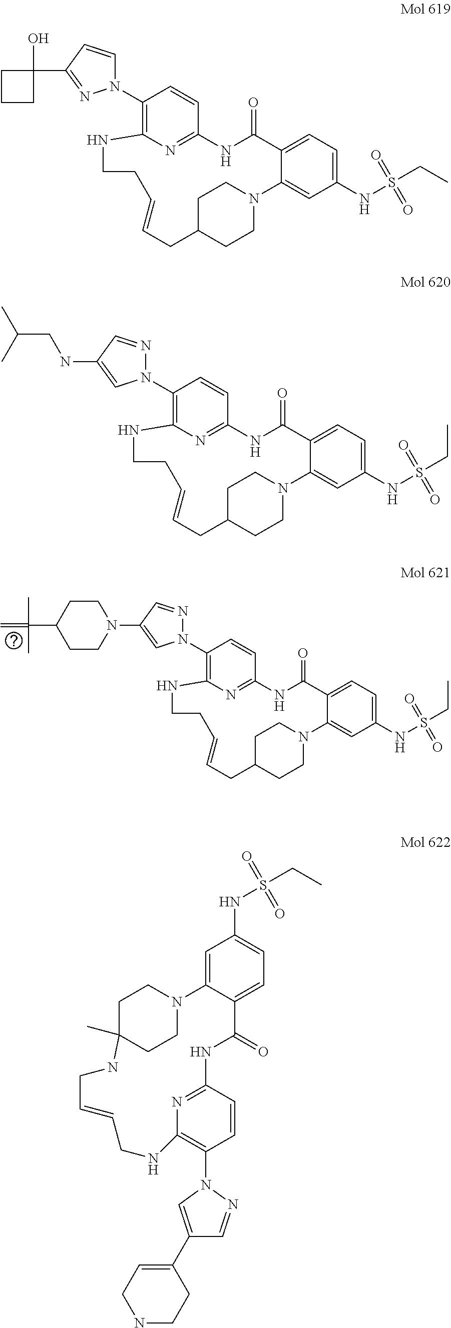

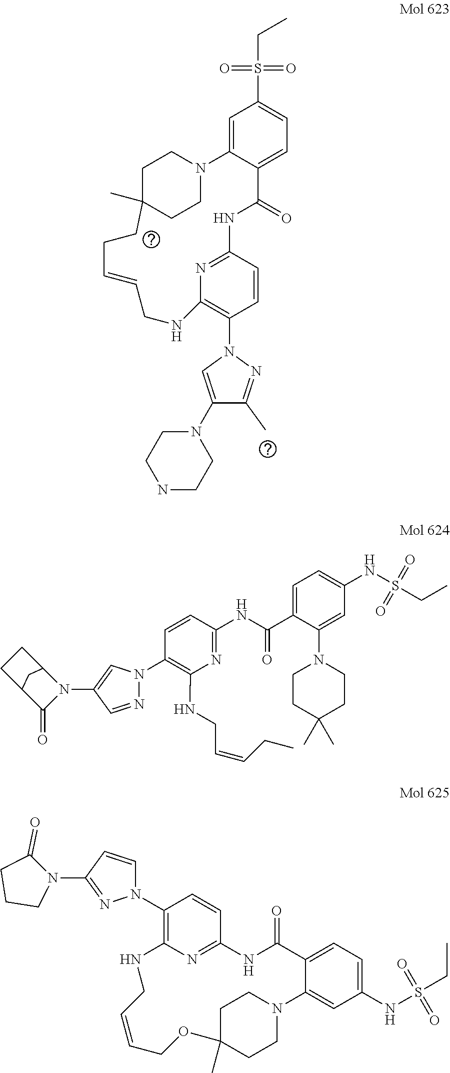 [SMILES: CCS(=O)(=O)c1ccc2c(c1)N1CCC(C)(CC/C=C/CNc3nc(ccc3-n3cc(N4CCNCC4)c(C)n3)NC2=O)CC1 ; CC/C=C\CNc1nc(NC(=O)c2ccc(NS(=O)(=O)CC)cc2N2CCC(C)(C)CC2)ccc1-n1cc(N2C(=O)C3CCC2C3)cn1 ; CCS(=O)(=O)Nc1ccc2c(c1)N1CCC(C)(CC1)OC/C=C\CNc1nc(ccc1-n1ccc(N3CCCC3=O)n1)NC2=O]

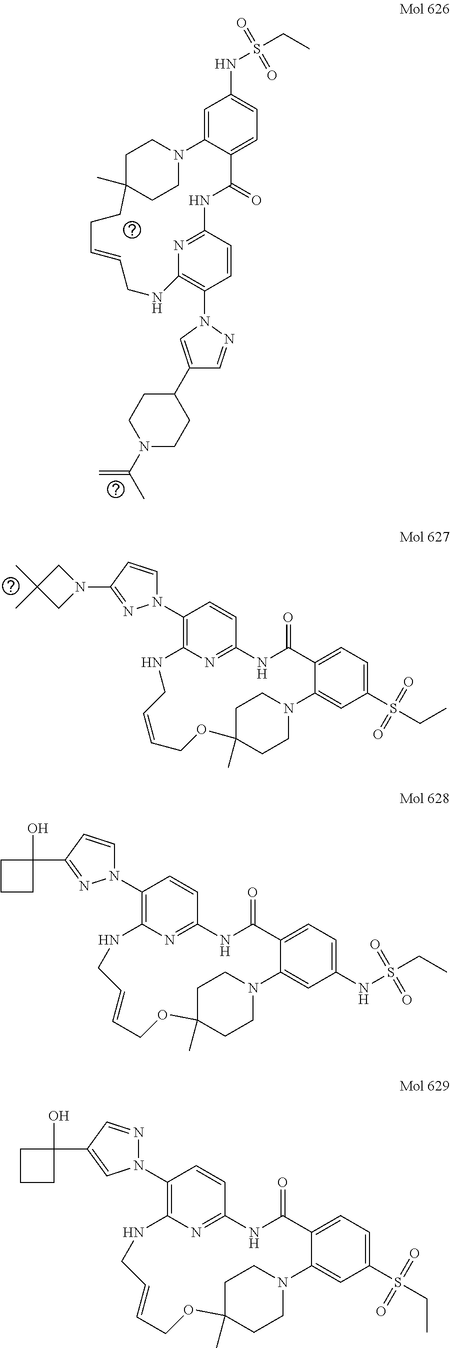 [SMILES: C=C(C)N1CCC(c2cnn(-c3ccc4nc3NC/C=C/CCC3(C)CCN(CC3)c3cc(NS(=O)(=O)CC)ccc3C(=O)N4)c2)CC1 ; CCS(=O)(=O)c1ccc2c(c1)N1CCC(C)(CC1)OC/C=C\CNc1nc(ccc1-n1ccc(N3CC(C)(C)C3)n1)NC2=O ; CCS(=O)(=O)Nc1ccc2c(c1)N1CCC(C)(CC1)OC/C=C/CNc1nc(ccc1-n1ccc(C3(O)CCC3)n1)NC2=O ; CCS(=O)(=O)c1ccc2c(c1)N1CCC(C)(CC1)OC/C=C/CNc1nc(ccc1-n1cc(C3(O)CCC3)cn1)NC2=O]

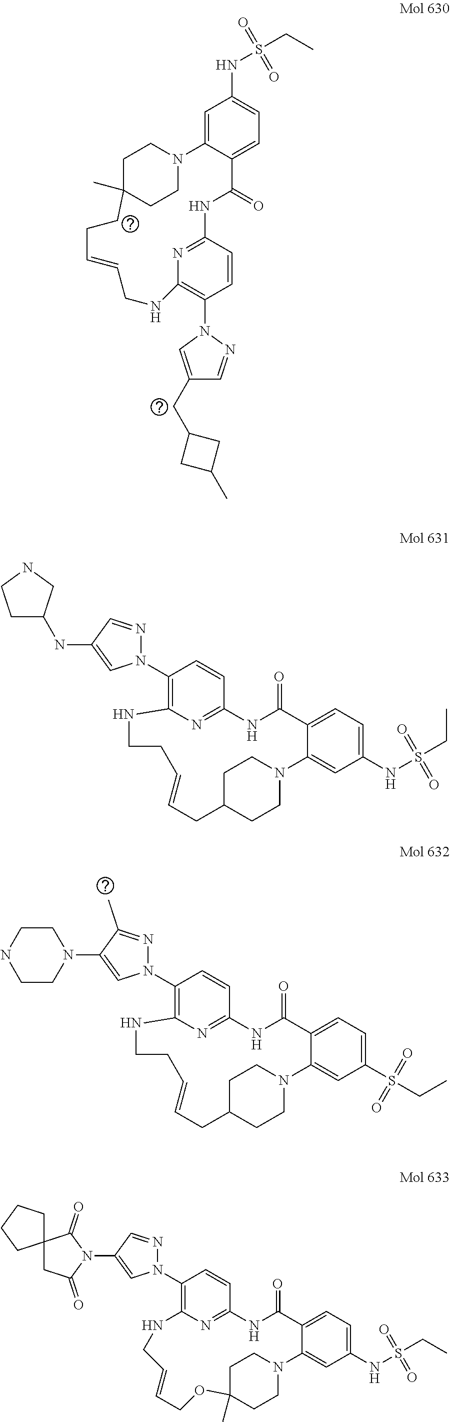 [SMILES: CCS(=O)(=O)Nc1ccc2c(c1)N1CCC(C)(CC/C=C/CNc3nc(ccc3-n3cc(CC4CC(C)C4)cn3)NC2=O)CC1 ; CCS(=O)(=O)Nc1ccc2c(c1)N1CCC(C/C=C/CCNc3nc(ccc3-n3cc(NC4CCNC4)cn3)NC2=O)CC1 ; CCS(=O)(=O)c1ccc2c(c1)N1CCC(C/C=C/CCNc3nc(ccc3-n3cc(N4CCNCC4)c(C)n3)NC2=O)CC1 ; CCS(=O)(=O)Nc1ccc2c(c1)N1CCC(C)(CC1)OC/C=C/CNc1nc(ccc1-n1cc(N3C(=O)CC4(CCCC4)C3=O)cn1)NC2=O]

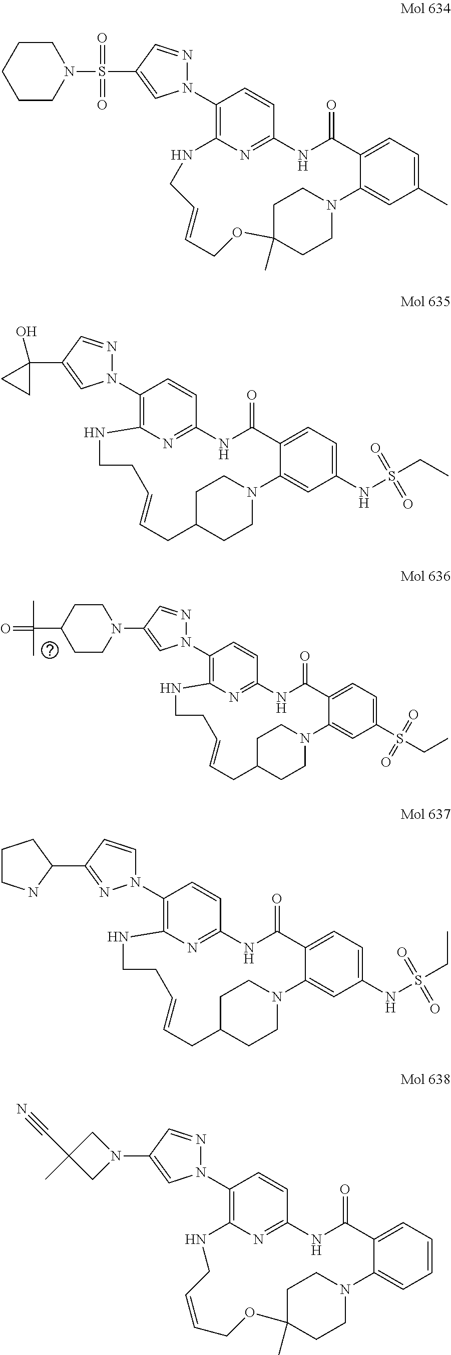

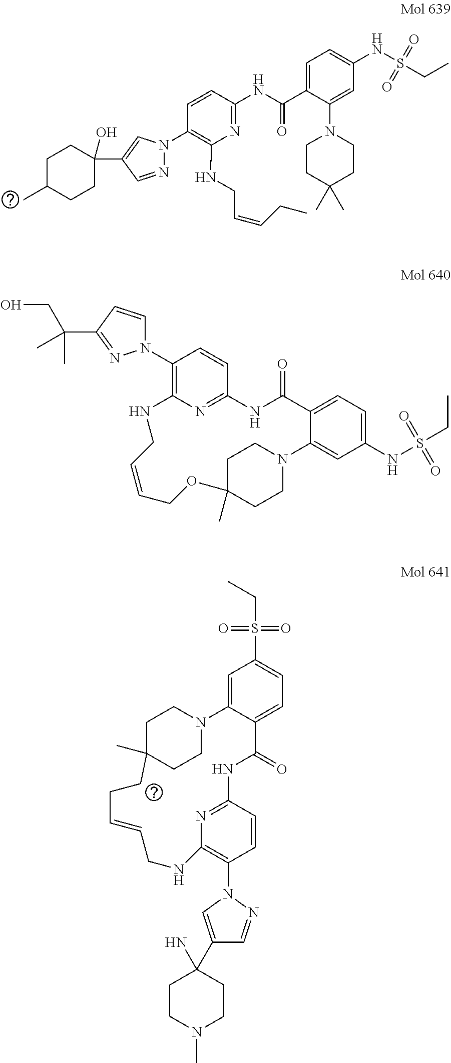 [SMILES: CC/C=C\CNc1nc(NC(=O)c2ccc(NS(=O)(=O)CC)cc2N2CCC(C)(C)CC2)ccc1-n1cc(C2(O)CCC(C)CC2)cn1 ; CCS(=O)(=O)Nc1ccc2c(c1)N1CCC(C)(CC1)OC/C=C\CNc1nc(ccc1-n1ccc(C(C)(C)CO)n1)NC2=O ; CCS(=O)(=O)c1ccc2c(c1)N1CCC(C)(CC/C=C/CNc3nc(ccc3-n3cc(C4(N)CCN(C)CC4)cn3)NC2=O)CC1]

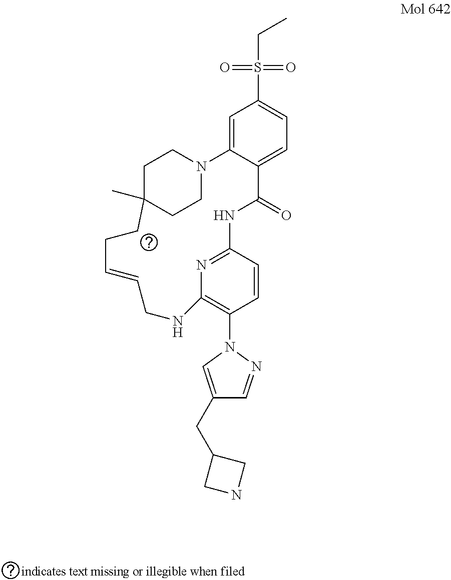 [SMILES: CCS(=O)(=O)c1ccc2c(c1)N1CCC(C)(CC/C=C/CNc3nc(ccc3-n3cc(CC4CNC4)cn3)NC2=O)CC1]

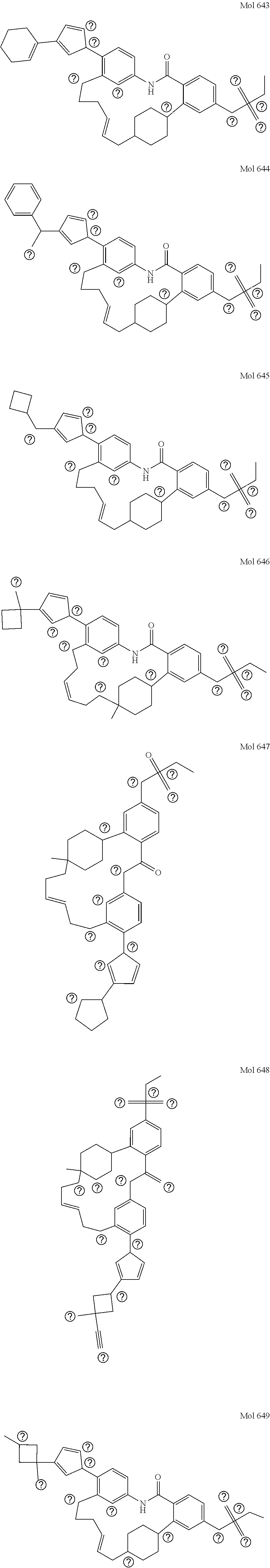

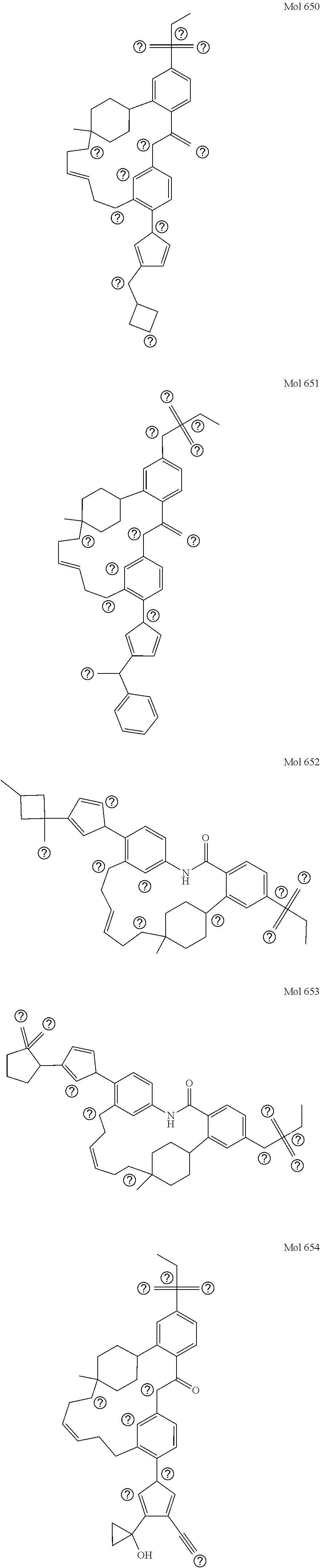

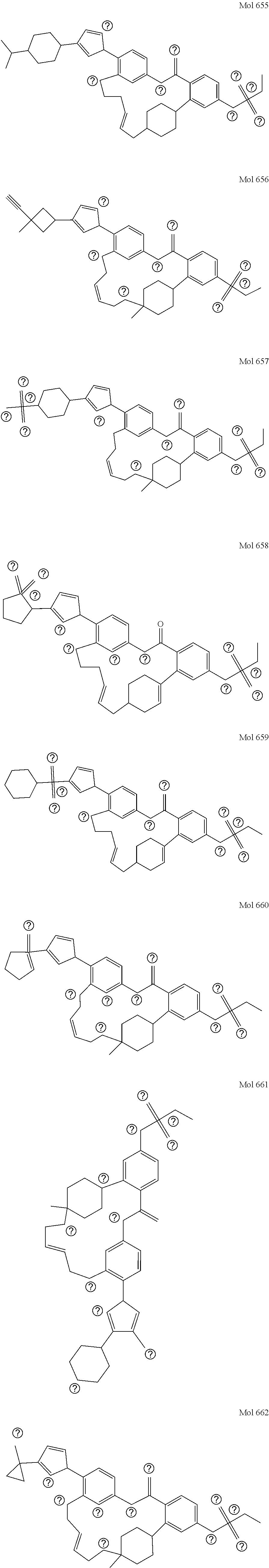

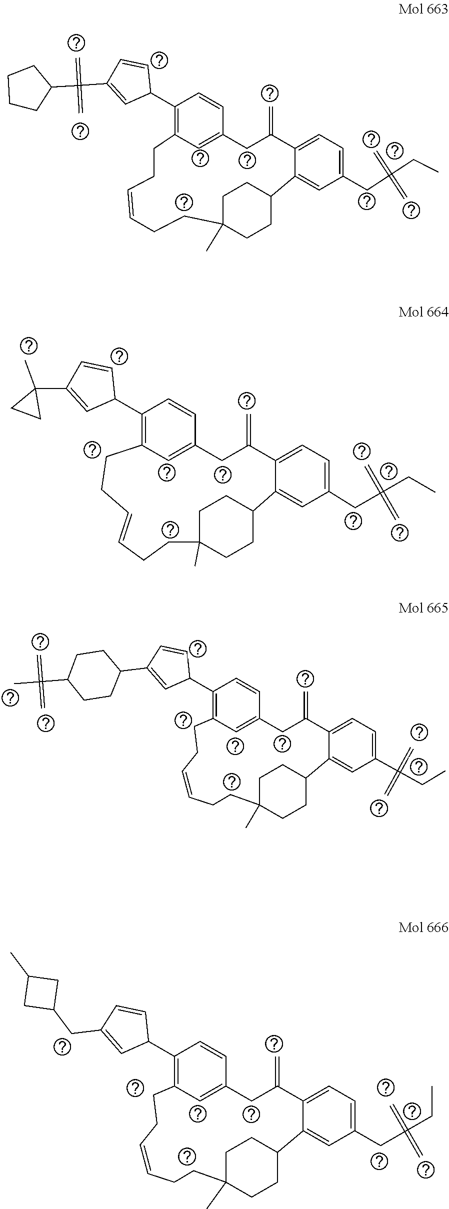

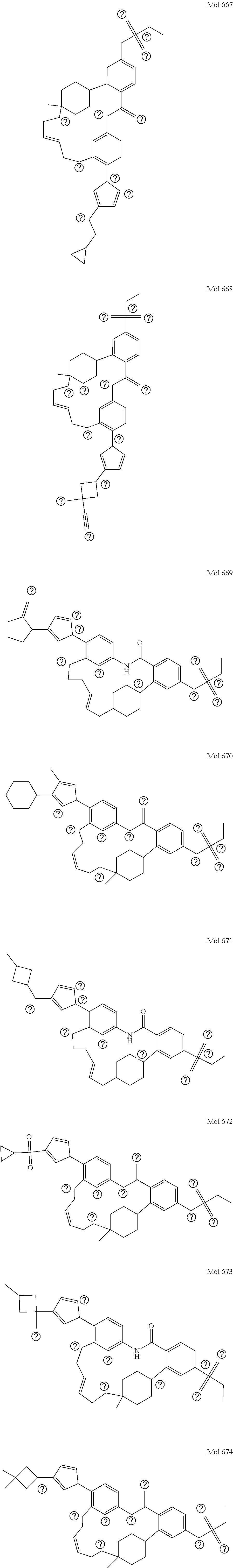

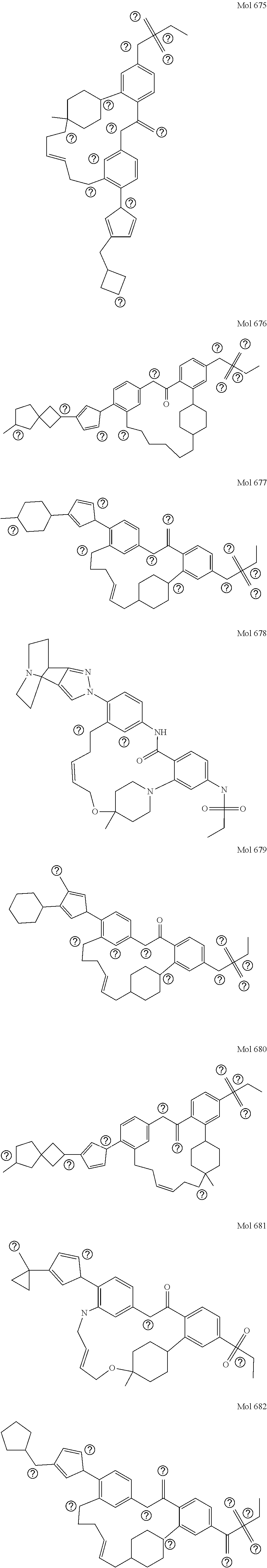

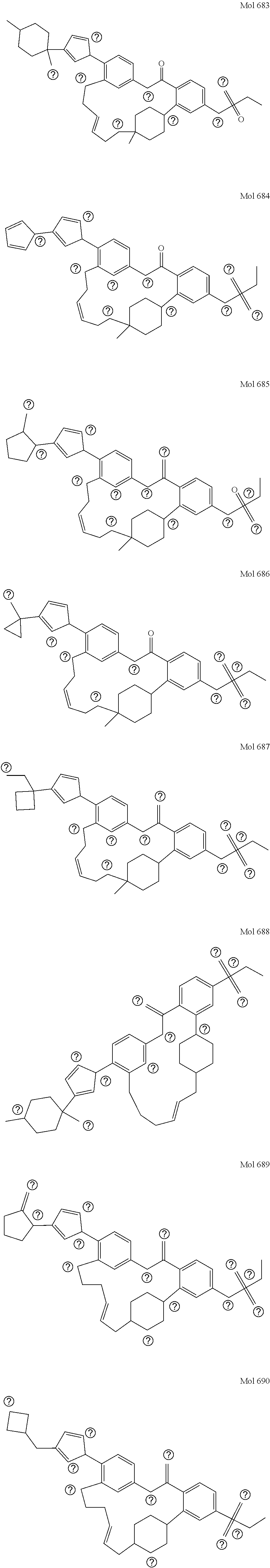

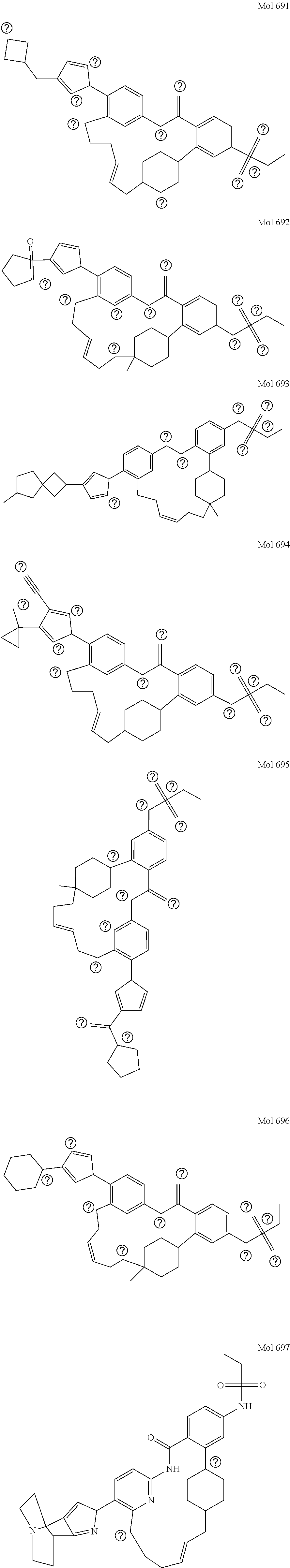

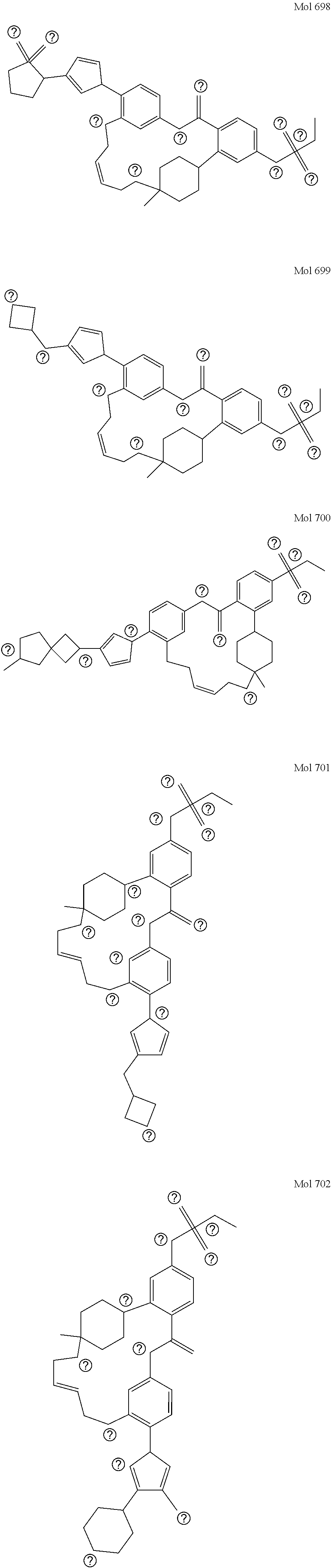

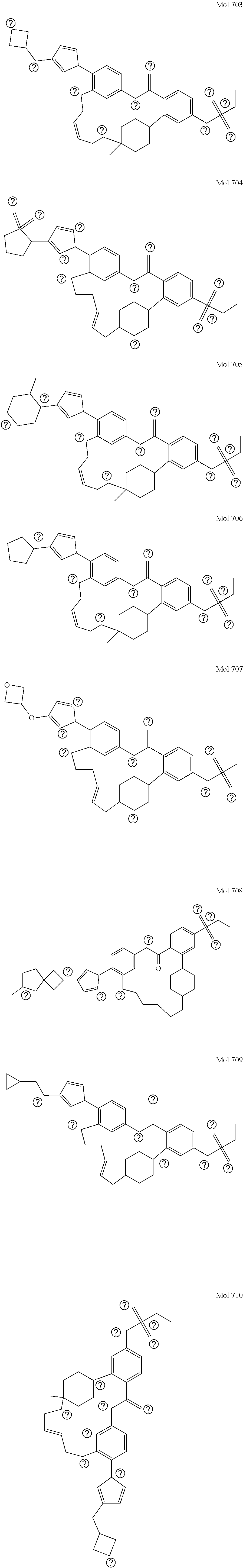

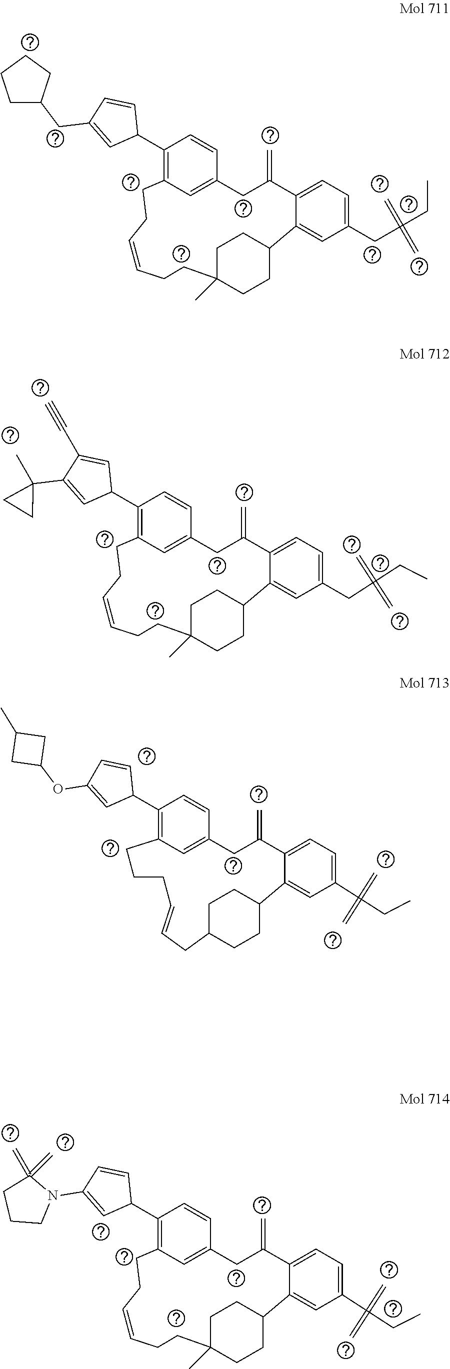

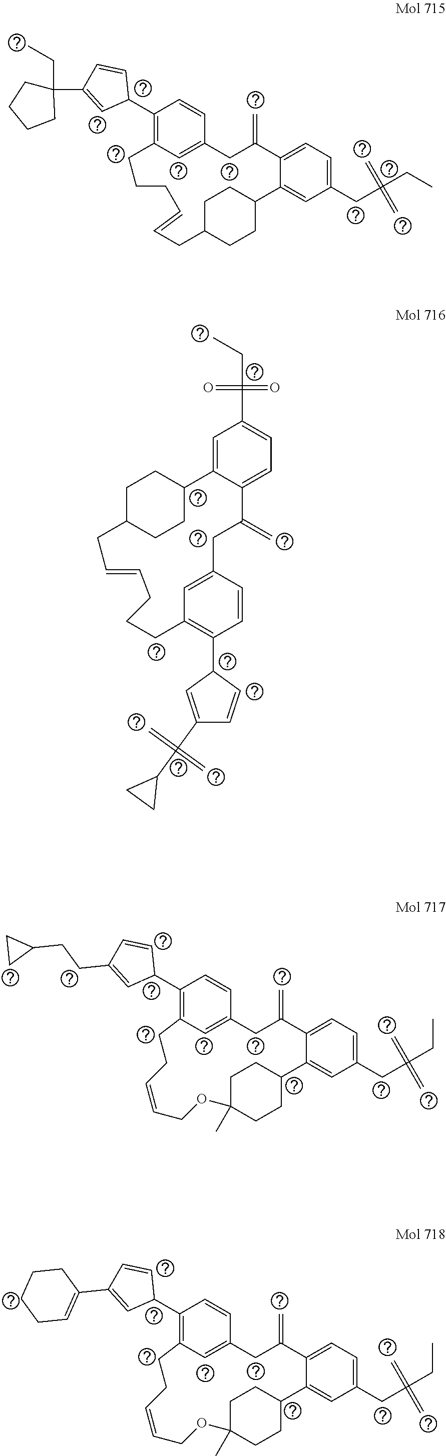

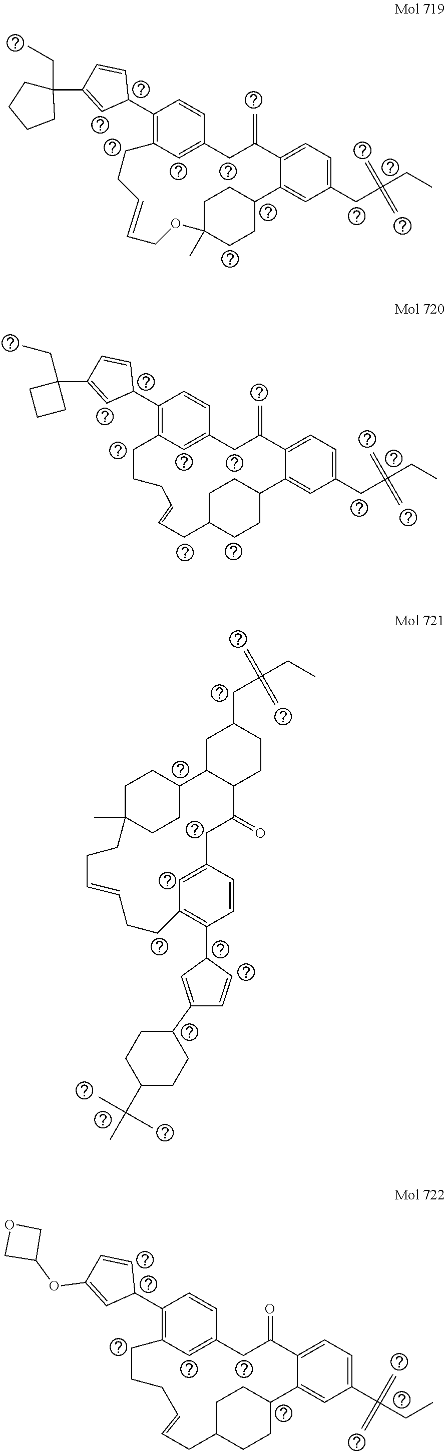

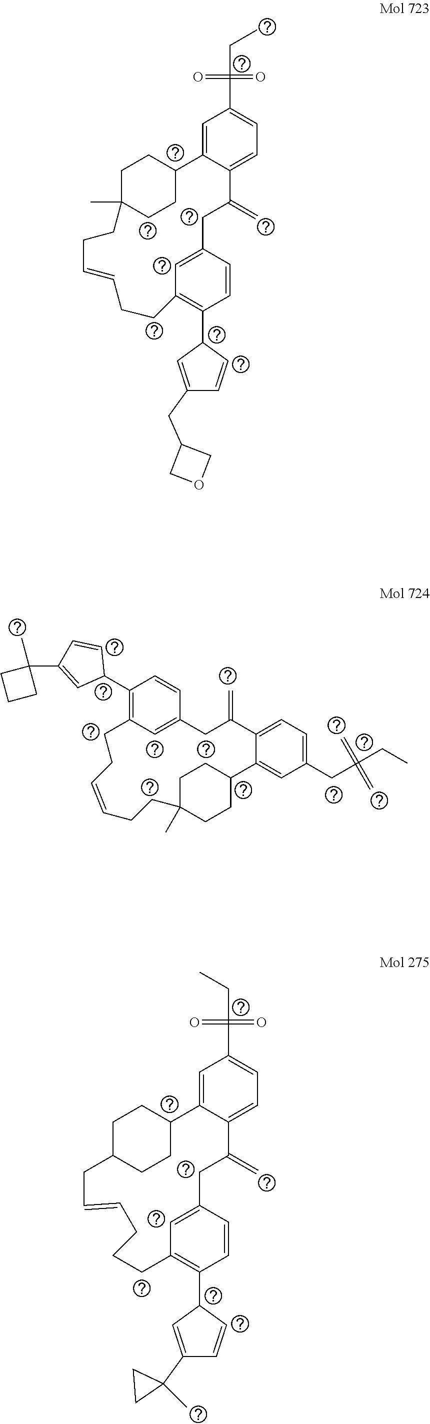

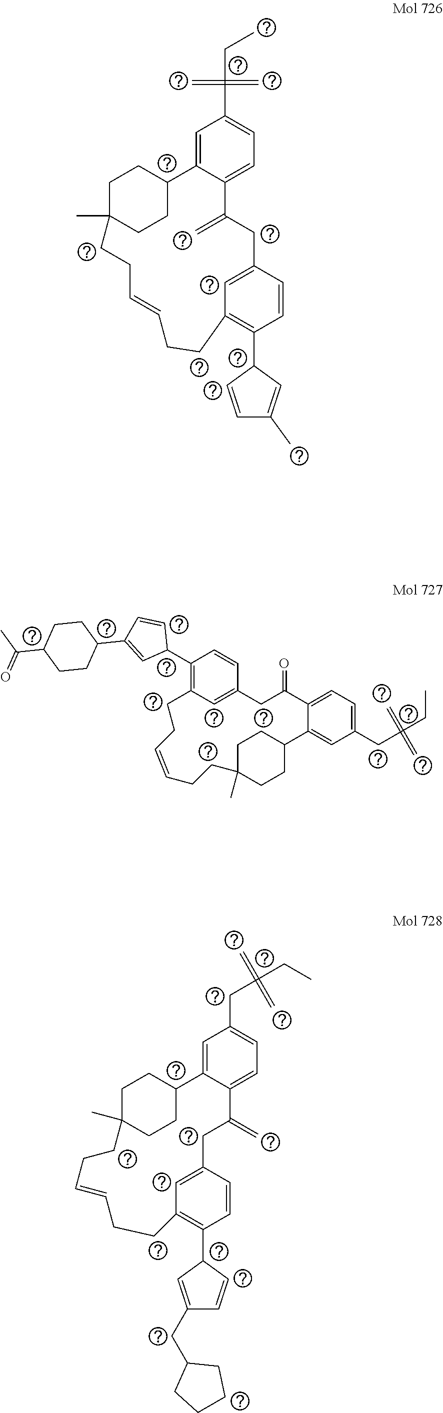

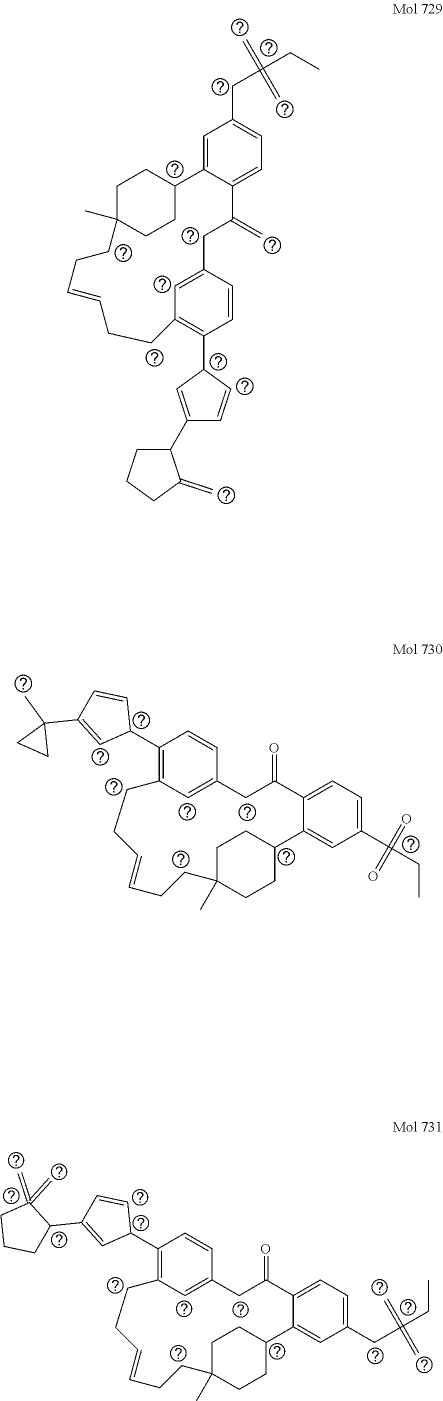

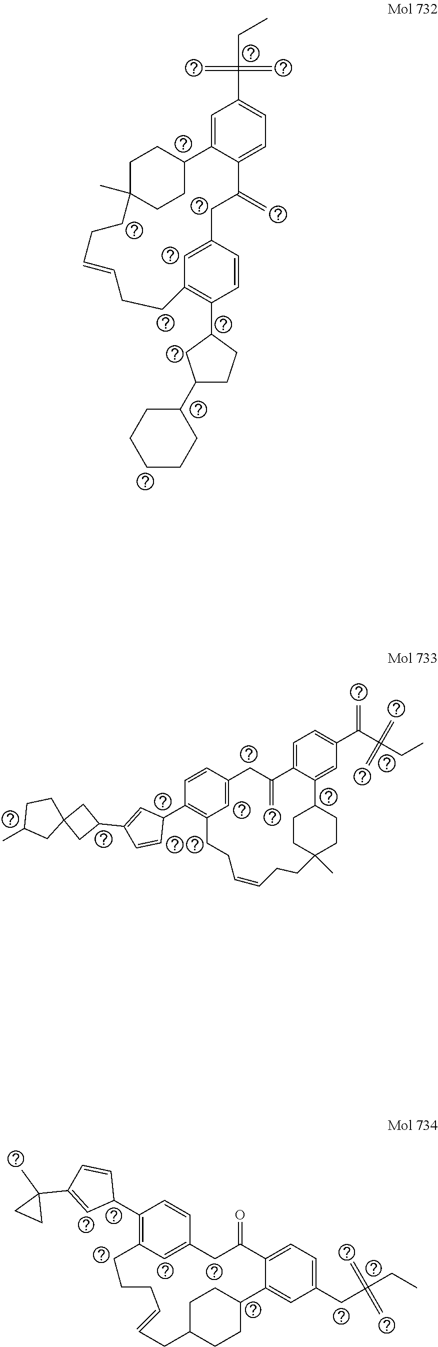

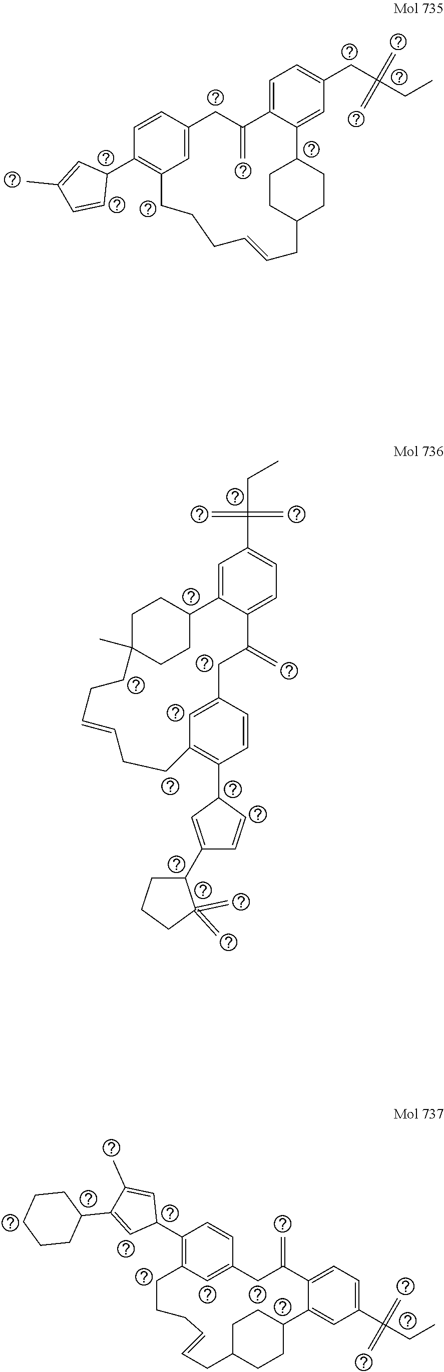

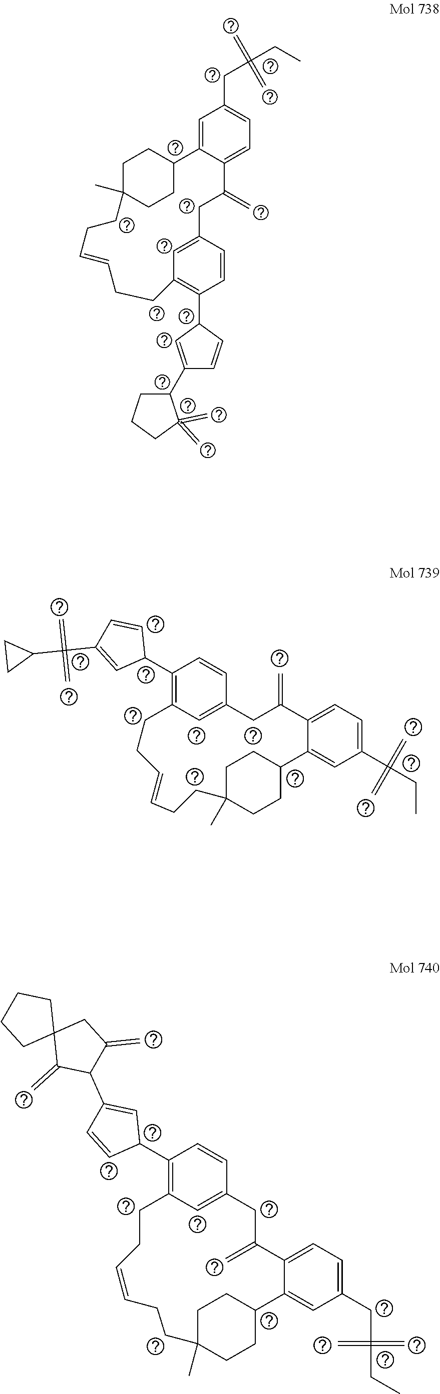

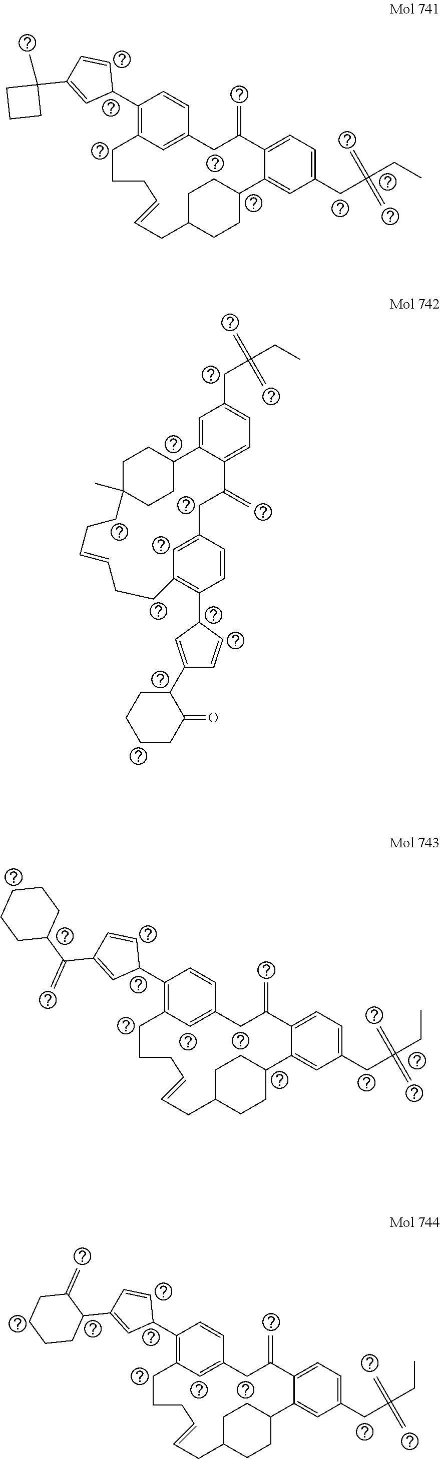

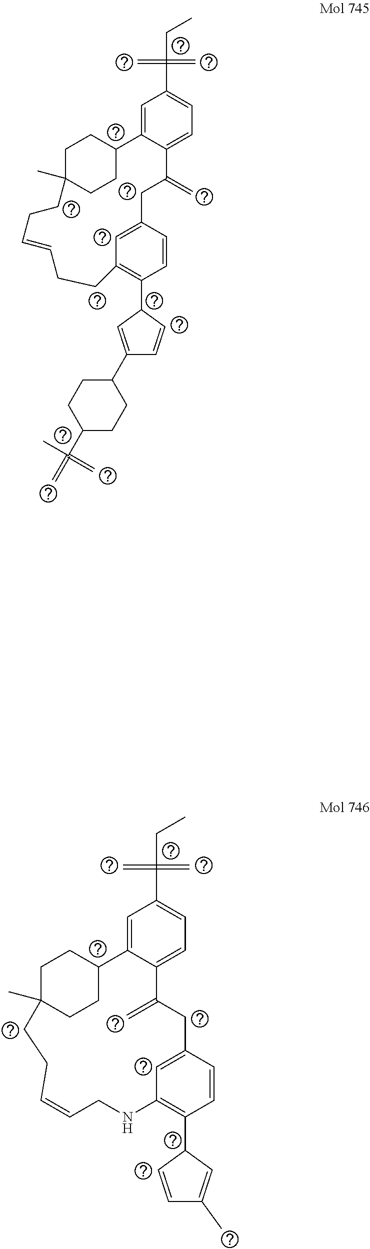

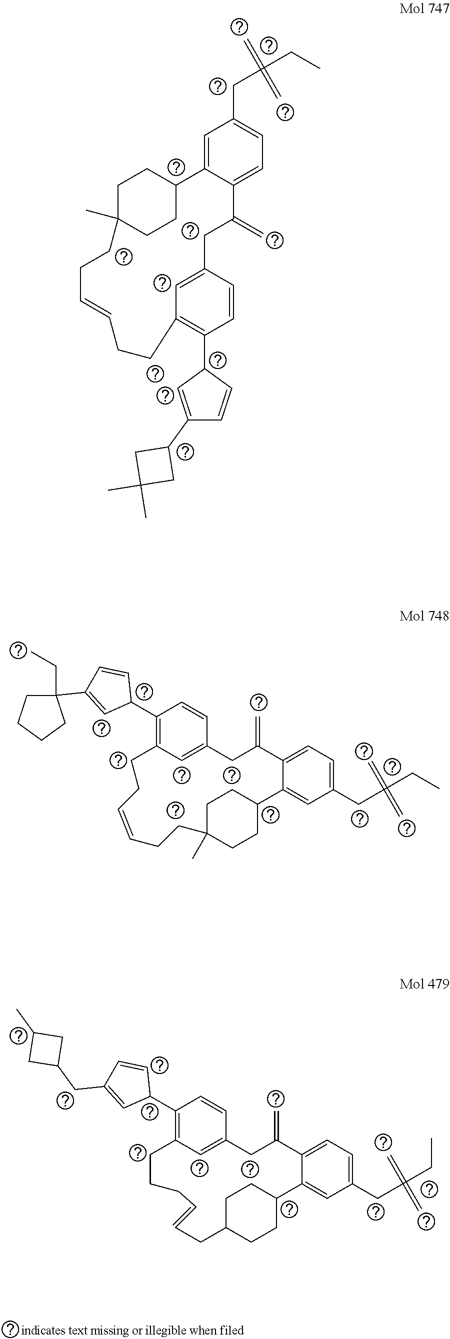

### Adducts

The compounds described herein, for example, the compounds of Formula (I) as described in the “Compound” section above, can be useful for forming an adduct with a KIF18A enzyme. The formation of an adduct between the compounds described herein and KIF18A can be useful for treating cancer. In some embodiments, the formation of the adduct inhibits the KIF18A protein, thereby treating cancer.

An adduct can comprise a KIFA18A protein and a compound of any Formula described herein.

The compounds described herein can interact or contact KIF18A when there is suitable adjacent distance between the compound and the KIF18A to form an adduct. In some embodiments, adjacent can refer to the distance of about 1 to 10 angstroms between the compound and KIF18A. In some embodiments, it can be about 1 to 8 angstroms between the compound and KIF18A. In some embodiments, it can be about 1 to 5 angstroms between the compound and KIF18A. In some embodiments, it can be about 3 to 5 angstroms between the compound and KIF18A.

The compounds described herein have structures which are useful for interacting with KIF18A to bind with it and form an adduct. For example, the 6-membered aromatic ring comprising Y¹, Y², and Y³ (hereinafter referred to as “Ring A”) can be sandwiched between the side chains of the KIF18A in the α4-helix and α6-helix location of the KIF18A. Aromatic rings which have a planar structure are useful for sandwiching between the α4-helix and α6-helix. Ring A can also be substituted with a diverse range of polar groups that can form hydrogen bonds that an interact with KIF18A, for example the substituent R³ as described herein. The amide linker between Ring A and the pyrazole substituted 6-membered heteroaromatic ring (hereinafter referred to as the “Ring B”) may be useful for keeping Ring A and Ring B in the same plane due to the carbonyl group allowing for delocalization of the aromatic rings. Ring B can interact against the flat surface of KIF18A α4-helix, and can also be useful for forming hydrogen bonds. The saturated heterocycloalkyl ring attached to Ring A (e.g., the piperidine ring in many examples; hereinafter referred to as “Ring C”) can interact with hydrophobic KIF18 residues. For example, Ring C can interact with the hydrophobic residues within the α4-helix and α6-helix region of KIF18A. Additionally, if Ring C is substituted with halogens, alkyls, or cycloalkyls, it can contact with a deeper layer of the hydrophobic side chains of the KIF18A β-sheet. The L¹-R¹ tether can be useful for interacting with the hydrophobic side chains of the KIF18A β-sheet region. Additionally, the L¹-R¹ tether can interact with KIF18A via hydrogen bonding in the α4-helix region. The L¹-R¹ tether region can weave through the α6-helix and β-sheet region of KIF18A and interact with the sidechains.

In some embodiments, Ring A is adjacent to the KIF18A between the KIF18 α4-helix and α6-helix. In some embodiments, Ring A is adjacent to the KIF18A between Leu285 residue and Tyr348 residue.

In some embodiments, Ring B is adjacent to a flat surface of the KIF18A α4-helix. In some embodiments, Ring B is adjacent to the KIF18A Gly289 residue or Asn293 residue. In some embodiments, Ring B interacts with the KIF18A Asn293 residue via a hydrogen bond.

In some embodiments, Ring C is adjacent to hydrophobic residues of the KIF18A. In some embodiments, the hydrophobic residues of the KIF18A are in α4-helix or α6-helix, or a combination thereof. In some embodiments, Ring C is adjacent to the KIF18A Tyr112 residue, Leu284 residue, Leu288 residue, and Tyr348 residue. In some embodiments, ring C is adjacent to the KIF18A Leu110 residue, Leu259 residue, Met328 residue, or a combination thereof.

In some embodiments, L¹-R¹ is adjacent to the hydrophobic side chain of the KIF18A α6-helix and β-sheet. L¹-R¹ is adjacent to the Ala352 residue, Met328 residue, and Tyr112 residue of KIF18A. In some embodiments, L¹-R¹ is adjacent to the hydrophobic side chain of the KIF18A β-sheet. In some embodiments, L¹-R¹ is adjacent to the KIF18A Leu110 residue, Leu259 residue, and Met328 residue.

In some embodiments, R¹ interacts with the KIF18A α4-helix via a hydrogen bond. In some embodiments, R¹ interacts with the KIF18A Asn293 residue via the hydrogen bond.

In some embodiments, R³ interacts with KIF18A via a hydrogen bond. In some embodiments, R³ interacts with the KIF18A Ser262 residue via a hydrogen bond.

Provided in some embodiments is an adduct comprising a KIF18A and a compound described herein, wherein the compound is a compound provided in Table 1.

### Compositions

The compounds described herein, for example, the compounds described in the “Compound” section above can be useful for forming a composition with a protein. In some embodiments, the composition comprising a protein and a compound described herein form an adduct. In some embodiments, the protein is KIF18A. In some embodiments, the compound can be any suitable compound described herein.

The composition can be useful for treating cancer. In some embodiments, the formation of the adduct inhibits the KIF18A protein, thereby treating cancer.

Also provided herein is a composition comprising an adduct described herein. In some embodiments, the adduct comprises KIF18A and a compound described herein. Accordingly, in some embodiments, provided herein is a composition comprising a protein and a compound, wherein the protein is KIF18A, and the compound is a compound described herein.

### Pharmaceutical Compositions

Provided in some embodiments herein is a pharmaceutical composition comprising a compound provided herein, or pharmaceutically acceptable salt, solvate, or isotopologue thereof. In some embodiments, the pharmaceutical composition comprises a pharmaceutically acceptable excipient.

Provided in some embodiments herein is a method for preparing a pharmaceutical composition described herein. In some embodiments, the method comprises mixing a pharmaceutically acceptable carrier and a compound provided herein, or pharmaceutically acceptable salt, solvate, or isotopologue thereof.

In some embodiments, the compounds described herein are formulated in oral dosage forms that include, by way of example only, tablets, powders, pills, dragees, capsules, liquids, gels, syrups, elixirs, slurries, suspensions and the like.

In some embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, tale, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In some embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as tale or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

The pharmaceutically acceptable excipients in the pharmaceutical composition described herein can be any suitable pharmaceutically acceptable excipient. In some embodiments, the pharmaceutically acceptable excipient is sodium lauryl sulfate, partially pre-gelatinized maize starch, microcrystalline cellulose, sodium starch glycolate, hydroxypropylcellulose LF, colloidal silicon dioxide, and magnesium stearate. In some embodiments, the sodium lauryl sulfate is Kolliphore SLS fine, the partially pre-gelatinized maize starch is Starch 1500, the microcrystalline cellulose is Avicel PH 101, the sodium starch glycolate is type A, the hydroxypropyl cellulose LF is Klucel HPC LF, and the colloidal silicon dioxide is Aerosil Pharma 200.

Provided herein is a pharmaceutical composition comprising a compound described herein, or pharmaceutically acceptable salt, solvate, or isotopologue thereof, and a pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

### Methods of Treatment

In some embodiments, a compound provided herein, or pharmaceutically acceptable salt, solvate, or isotopologue thereof is used in a method for treating cancer.

Provided in some embodiments herein is a method for treating cancer in an individual, the method comprising administering to the individual a compound provided herein or pharmaceutically acceptable salt, solvate, or isotopologue thereof.

Provided in some embodiments is a method of inhibiting KIF18A, comprising forming an adduct by contacting KIF18A with any compound of Formula (B). The adduct can comprise any adduct described herein. In some embodiments, the adduct comprising a KIF18A and a compound as provided in Table 1.

Provided in some embodiments in a method of treating cancer in a patient by forming an adduct as described herein.

Cancer is often characterized by unregulated cell proliferation. Damage to one or more genes, responsible for the cellular pathways, which control progress of proliferation through the cell cycle and centrosome cycle, can cause the loss of normal regulation of cell proliferation. These deregulated genes can code for various tumor suppressor or oncogene proteins, which participate in a cascade of events, leading to unchecked cell-cycling progression and cell proliferation. Various kinase and kinesin proteins have been identified, which play key roles in cell cycle and mitotic regulation and progression of normal dividing cells and cancer cells.

In some embodiments, the cancer is mediated by KIF18A. In some embodiments, the cancer is mediated by a misregulation of KIF18A. In some embodiments, the cancer is treated by modulating KIF18A. In some embodiments, the cancer is treated by inhibiting KIF18A.

Provided in some embodiments herein is a method of inhibiting KIF18A with a compound provided herein, or pharmaceutically acceptable salt, solvate, or isotopologue thereof. In some embodiments, the method comprises contacting KIF18A with the compound.

KIF18A belongs to the Kinesin-8 subfamily and is a plus-end directed motor protein. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells. KIF18A is overexpressed in various types of cancers, including but not limited to colon, breast, lung, pancreas, prostate, bladder, head, neck, cervix, and ovarian cancers. Further, genetic deletion or knockdown, or inhibition of KIF18A affects the mitotic spindle apparatus in cancer cell lines. Particularly, inhibition of KIF18A has been found to induce mitotic cell arrest, a known vulnerability that can promote cell death in mitosis via apoptosis, mitotic catastrophe, or multipolarity driven lethality or death after mitotic slippage in interphase.

In some embodiments, the cancer is (a) a solid or hematologically derived tumor selected from cancer of the bladder, endometrial, lung squamous cell, breast, colon, kidney, liver, lung, small cell lung cancer, esophagus, gall-bladder, brain, head and neck, ovary, pancreas, stomach, cervix, thyroid, prostate and skin, (b) a hematopoietic tumor of lymphoid lineage selected from leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma, (c) a hematopoietic tumor of myeloid lineage selected from acute and chronic myelogenous leukemias, myelodysplastic syndrome and promyelocytic leukemia (d) a tumor of mesenchymal origin selected from fibrosarcoma and rhabdomyosarcoma, (e) a tumor of the central and peripheral nervous system selected from astrocytoma, neuroblastoma, glioma and schwannoma, or (f) a melanoma, seminoma, teratocarcinoma, osteosarcoma, xeroderma pigmentosum, keratoacanthoma, thyroid follicular cancer or Kaposi's sarcoma.

In some embodiments, the cancer is bladder cancer, endometrial cancer, lung squamous cell cancer, breast cancer, colon cancer, kidney cancer, liver cancer, lung cancer, small cell lung cancer, esophagus cancer, gall-bladder cancer, brain cancer, head and neck cancer, ovarian cancer, pancreas cancer, stomach cancer, cervix cancer, thyroid cancer, prostate cancer or skin cancer. In some embodiments, the cancer is leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma or Burkett's lymphoma. In some embodiments, the cancer is acute and chronic myelogenous leukemias, myelodysplastic syndrome or promyelocytic leukemia. In some embodiments, the cancer is fibrosarcoma or rhabdomyosarcoma. In some embodiments, the cancer is melanoma, seminoma, teratocarcinoma, osteosarcoma, xeroderma pigmentosum, keratoacanthoma, thyroid follicular cancer or Kaposi's sarcoma. In some embodiments, the cancer is melanoma, prostate cancer, ovarian cancer, cervical cancer, breast cancer, colon cancer, sarcoma, or leukemia.

In treatment methods according to embodiments of the invention, an effective amount of at least one compound described herein is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Amino” refers to the —NH₂ radical.

“Cyano” refers to the —CN radical.

“Nitro” refers to the —NO2 radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₈ alkyl). In other embodiments, an alkyl comprises three to five carbon atoms (e.g., C₃-C₈ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. In some instances, an alkyl group provided herein is substituted or further substituted with one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR, —C(O)N(R^{a})₂, —N(R^{a})C(O)OR^{a}, —OC(O)—N(R^{a})₂, —N(R^{a})C(O)R^{a}, —N(R^{a})S(O)ₜR^{a}(where t is 1 or 2), —S(O)ₜOR^{a}(where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Hydroxyalkyl” refers to an alkyl radical, as defined above, that is substituted by one or more hydroxy radicals.

“Alkoxy” refers to a radical bonded through an oxygen atom of the formula —O-alkyl, where alkyl is an alkyl group as defined above.

“Aminoalkyl” refers to a radical bonded through a nitrogen atom of the formula —NH-alkyl, where alkyl is an alkyl group as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group provided herein is substituted or further substituted as described for “alkyl” hereinabove.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group provided herein is substituted or further substituted as described for “alkyl” hereinabove.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₈ alkylene). In other embodiments, an alkylene comprises three to five carbon atoms (e.g., C₃-C₈ alkylene). Unless stated otherwise specifically in the specification, an alkylene chain is substituted or further substituted as described for “alkyl” hereinabove.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₈ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₈ alkenylene). Unless stated specifically otherwise in the specification, the alkenylene is substituted or further substituted as described for “alkyl” hereinabove.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₈ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₈ alkynylene). Unless stated specifically otherwise in the specification, the alkynylene is substituted or further substituted as described for “alkyl” hereinabove.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some instances, an aryl group provided herein is substituted or further substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. Unless stated specifically otherwise in the specification, the alkylene chain part of the aralkyl radical is substituted or unsubstituted as described above for an alkylene chain. Unless stated specifically otherwise in the specification, the aryl part of the aralkyl radical is substituted or unsubstituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. In some instances, a carbocyclyl group provided herein is substituted or further substituted with one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a}(where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. Unless stated specifically otherwise in the specification, the alkylene chain and the carbocyclyl radical are independently substituted or further substituted as defined hereinabove.

“Carbocyclylalkenyl” refers to a radical of the formula —R^{c}-carbocyclyl where R^{c} is an alkenylene chain as defined above. Unless stated specifically otherwise, the alkenylene chain and the carbocyclyl radical are independently substituted or further substituted as defined hereinabove.

“Carbocyclylalkoxy” refers to a radical bonded through an oxygen atom of the formula —O—R^{c}-carbocyclyl where R^{c} is an alkylene chain as defined above. Unless stated specifically otherwise, the alkylene chain and the carbocyclyl radical are independently substituted or further substituted as defined hereinabove.

“Halo” or “halogen” refers to fluoro, bromo, chloro, or iodo substituents. In some embodiments, “halogen” refers to fluoro, bromo, or chloro substituents. In some embodiments, “halogen” refers to fluoro or chloro substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a “fluoroalkyl,” such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is substituted or further substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁s heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated specifically otherwise, a heteroalkyl group is substituted or further substituted as defined above for an alkyl group.

“Heteroalkylene” refers to a divalent heteroalkyl group defined above which links one part of the molecule to another part of the molecule. Unless stated specifically otherwise, a heteroalkylene is substituted or further substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some instances, a heterocyclyl group is substituted or further substituted with one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a}(where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. Unless stated specifically otherwise in the specification, the heterocyclyl part of the heterocyclylalkyl radical is substituted or further substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. Unless stated specifically otherwise in the specification, the heterocyclyl part of the heterocyclylalkoxy radical is substituted or further substituted as defined above for a heterocyclyl group.

“Heteroaryl” or “aromatic heterocycle” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) 7t-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, pyrazinone, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some instances, a heteroaryl group is substituted or further substituted with one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. Unless specifically stated otherwise in the specification, the heteroaryl part of the heteroarylalkyl radical is substituted or further substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. Unless specifically stated otherwise in the specification, the heteroaryl part of the heteroarylalkoxy radical is substituted or further substituted as defined above for a heteroaryl group.

“Adduct” refers to the combined product of two or more compounds and/or proteins to form a larger structure. For example, the adduct may comprise a protein and a compound described herein which forms a new singular structure. The protein and the compound may bind by protein-compound interactions which include, but are not limited to electrostatic interactions, van der Waals forces, hydrophobic interactions, hydrogen bonding, or covalent bonding.

“Adjacent” refers to being within a specific space and distance of a compound or protein. For example, a compound adjacent to a protein can be within a specific distance such as 20 angstroms or less, or 10 angstroms or less between the compound and protein. In some embodiments, a compound adjacent lies within a distance of about 1 to 6 Angstroms. In some embodiments, a compound adjacent lies within a distance of about 2 to 5 Angstroms. In some embodiments, a compound adjacent lies within a distance of about 3 to 5 Angstroms. In some embodiments, a compound adjacent lies within a distance of about 3 to 6 Angstroms.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). In some instances, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a}(where t is 1 or 2), —S(O)ₜOR^{a}(where t is 1 or 2), —S(O)ₜR^{a}(where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl). In some embodiments herein, an optionally substituted group is a group that is substituted by one or more groups independently selected at each occurrence from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, hydroxyalkyl, aminoalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one, two, or three of the preceding groups. In some embodiments, substituted groups are substituted with one or two of the preceding groups. In some embodiments, an optional substituent on an aliphatic carbon atom (acyclic or cyclic) includes oxo (═O). As a non-limiting example, in some embodiments herein, if any group described herein is substituted then it is substituted by 1, 2, or 3 groups independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, if any group described herein is substituted then it is substituted by 1, 2, or 3 groups independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups.

The compounds disclosed herein, in some embodiments, are used in different enriched isotopic forms, e.g., enriched in the content of ²H, ³H, ¹¹C, ¹³C and/or ¹⁴C. That is, compounds disclosed herein, in some embodiments, are isotopologues. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy of drugs, thus increasing the duration of action of drugs.

Unless otherwise stated, compounds described herein are intended to include compounds which differ only in the presence of one or more isotopically enriched atoms. For example, compounds having the present structures except for the replacement of one or more proton(s) by one or more deuterium(deuteria) or tritium(tritia), or combinations thereof, or except for the replacement of one or more ¹²C atom(s) in the structure by one or more ¹³C atom(s), one or more ¹⁴C atom(s), or combinations thereof, in the structure are within the scope of the present disclosure.

The compounds of the present disclosure optionally comprise unnatural proportions of atomic isotopes at one or more atom(s) that constitute such compounds. For example, the compounds may be labeled with one or more isotope(s), such as for example, deuterium (²H), tritium (³H), iodine-125 (¹²⁵I) or carbon-14 (¹⁴C). Isotopic substitution with ²H, ³H, ¹¹C, ¹³C, ¹⁴C, ¹⁵C, ¹²N, ¹³N, ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium-substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as MilliporeSigma.

The section headings used herein are for organizational purposes only and are not to be construed as limiting the subject matter described.

### SPECIFIC EMBODIMENTS OF THE INVENTION

Embodiment A1. A compound having the structure of Formula (I):

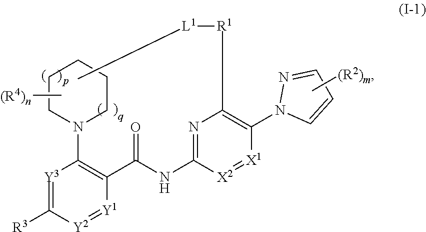

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -Lia-L^{1b}-*, where * designates the attachment point to R¹;
   L^{1a} is a bond, —O—, —NH—, —S—, or —SO₂—;
   L^{1b} is optionally substituted C₄-C₅ alkenylene;
R¹ is a bond, —O—, —NH—, or —N(C₁-C₄ alkyl)-;
each R² is independently halogen, —CN, —OR^{2′}, —N(R^{2a})₂, —C(O)N(R^{2a})₂, —N(R^{2a})C(O)NR^{2a}, —C(O)R², —C(O)OR^{2′}, —S(O)₂N(R^{2a})₂, —S(O)₂R^{2b}, optionally substituted C₁-C₈ alkyl, C₁-C₅ hydroxyalkyl, optionally substituted C₃-C₈ cycloalkyl, or optionally substituted 4- to 8-membered heterocycloalkyl;
   each R^{2a} is independently hydrogen, optionally substituted C₁-C₆ alkyl, or optionally substituted C₃-C₆ cycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl; R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6′}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment A2. The compound of Embodiment A1, or a pharmaceutically acceptable salt or solvate thereof, wherein:
   Y¹ is CR⁵;
   Y² is CR⁵;
   Y³ is CR⁵; and
   each R⁵ is independently hydrogen, halogen, —CN, —OR^{5a}, —N(R^{5a})₂, or C₁-C₈ alkyl.
Embodiment A3. The compound of Embodiment A1 or Embodiment A2, or a pharmaceutically acceptable salt or solvate thereof, wherein:
   X¹ is CR⁶ or N;
   X² is CR⁶; and
   each R⁶ is independently hydrogen, halogen, —CN, —OR^{5a}, —N(R^{5a})₂, or C₁-C₈ alkyl.
Embodiment A4. The compound of any one of Embodiments A1 to A3, or a pharmaceutically acceptable salt or solvate thereof, wherein:
   p is 1.
Embodiment A5. The compound of any one of Embodiments A1 to A4, or a pharmaceutically acceptable salt or solvate thereof, wherein:
   q is 1.
Embodiment A6. The compound of Embodiment A1, having the structure of Formula (II):

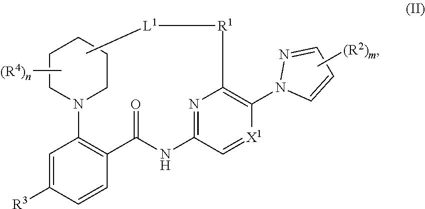

or a pharmaceutically acceptable salt or solvate thereof, wherein:
X¹ is CH or N.
Embodiment A7. The compound of any one of Embodiments A1 to A6, or a pharmaceutically acceptable salt or solvate thereof, wherein:
   R¹ is —NH— or —N(C₁-C₄ alkyl)-.
Embodiment A8. The compound of any one of Embodiments A1 to A7, or a pharmaceutically acceptable salt or solvate thereof, wherein:
   R¹ is —NH—.
Embodiment A9. The compound of any one of Embodiments A1 to A8, or a pharmaceutically acceptable salt or solvate thereof, wherein:
   L^{1a} is a bond or —O—.
Embodiment A10. The compound of any one of Embodiments A1 to A9, or a pharmaceutically acceptable salt or solvate thereof, wherein:
   L^{1a} is a bond.
Embodiment A11. The compound of Embodiment A10, or a pharmaceutically acceptable salt or solvate thereof, wherein:
   L^{1b} is C₅ alkenylene.
Embodiment A12. The compound of any one of Embodiments A1 to A9, or a pharmaceutically acceptable salt or solvate thereof, wherein:
   L^{1a} is —O—.
Embodiment A13. The compound of Embodiment A12, or a pharmaceutically acceptable salt or solvate thereof, wherein:
   L^{1b} is C₄ alkenylene.
Embodiment A14. The compound of any one of Embodiments A1 to A8, or a pharmaceutically acceptable salt or solvate thereof, wherein:
   L^{1a} is a bond; and L^{1b} is C₅ alkenylene;
   or L^{1a} is —O—; and L^{1b} is C₄ alkenylene.
Embodiment A15. The compound of any one of Embodiments A1 to A8, or a pharmaceutically acceptable salt or solvate thereof, wherein L¹ is —CH₂CH═CHCH₂CH₂—*, —CH₂CH₂CH═CHCH₂—*, or —OCH₂CH═CHCH₂—*, where * designates the attachment point to R¹.
Embodiment A16. The compound of Embodiment A1, having the structure of Formula (IIIa) or (IIIb):

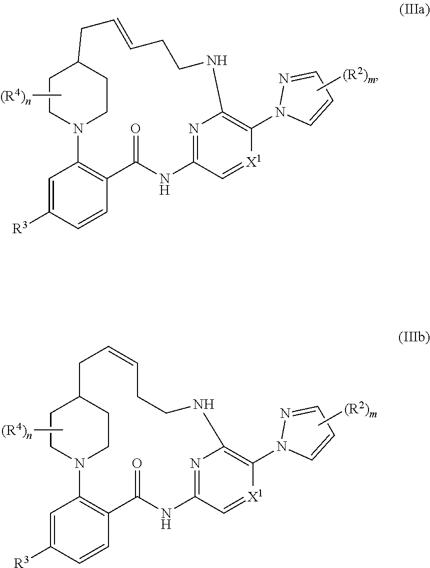

or a pharmaceutically acceptable salt or solvate thereof, wherein:
X¹ is CH or N.
Embodiment A17. The compound of any one of Embodiments A1 to A16, or a pharmaceutically acceptable salt or solvate thereof, wherein:
   X¹ is CH.
Embodiment A18. The compound of any one of Embodiments A1 to A16, or a pharmaceutically acceptable salt or solvate thereof, wherein:
   X¹ is N.
Embodiment A19. The compound of any one of Embodiments A1 to A18, or a pharmaceutically acceptable salt or solvate thereof, wherein:
   each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl;
   each R^{2a} is independently hydrogen or C₁-C₆ alkyl; wherein the alkyl is optionally substituted with 1, 2, or 3 groups independently selected from halogen, —OH, and —CN;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl;
   and
   each R^{2b} is independently C₁-C₆ alkyl.
Embodiment A20. The compound of any one of Embodiments A1 to A19, or a pharmaceutically acceptable salt or solvate thereof, wherein:
   each R² is independently —F, —Cl, —CN, —OR^{2a}, —C(O)N(R^{2a})₂, —S(O)₂R^{2b}, or C₁-C₄ hydroxyalkyl;
   each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH;
   or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and
   each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment A21. The compound of any one of Embodiments A1 to A20, or a pharmaceutically acceptable salt or solvate thereof, wherein:
   each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

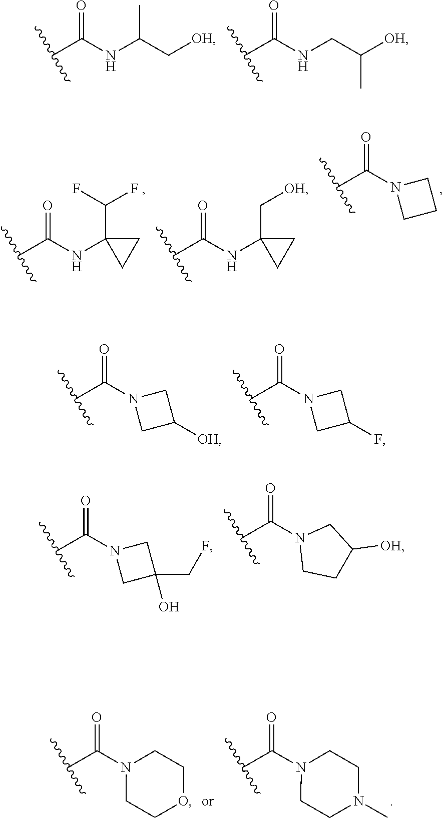

Embodiment A22. The compound of any one of Embodiments A1 to A21, or a pharmaceutically acceptable salt or solvate thereof, wherein:
   m is 1.
Embodiment A23. The compound of any one of Embodiments A1 to A22, or a pharmaceutically acceptable salt or solvate thereof, wherein:
   each R⁴ is independently halogen, —CN, C₁-C₆ alkyl, or C₁-C₆ fluoroalkyl.
Embodiment A24. The compound of any one of Embodiments A1 to A23, or a pharmaceutically acceptable salt or solvate thereof, wherein:
   each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment A25. The compound of any one of Embodiments A1 to A24, or a pharmaceutically acceptable salt or solvate thereof, wherein:
   each R⁴ is independently —CN or —CH₃.
Embodiment A26. The compound of any one of Embodiments A1 to A25, or a pharmaceutically acceptable salt or solvate thereof, wherein:
   n is 1.
Embodiment A27. The compound of any one of Embodiments A1 to A26, or a pharmaceutically acceptable salt or solvate thereof, wherein:
   R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or C₁-C₆ alkyl;
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl.
Embodiment A28. The compound of any one of Embodiments A1 to A27, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
   or R^{3a} and R^{3b} are taken together with the intervening atoms to which they are attached to form an optionally substituted 5- to 6-membered heterocycloalkyl.
Embodiment A29. The compound of Embodiment A28, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment A30. The compound of Embodiment A29, or a pharmaceutically acceptable salt or solvate thereof, wherein:
   R^{3a} is hydrogen or C₁-C₃ alkyl; and
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment A31. The compound of claim 28, or a pharmaceutically acceptable salt or solvate thereof, wherein:
   R^{3a} is hydrogen, —CH₃, or —CH₂CH₃; and
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH,

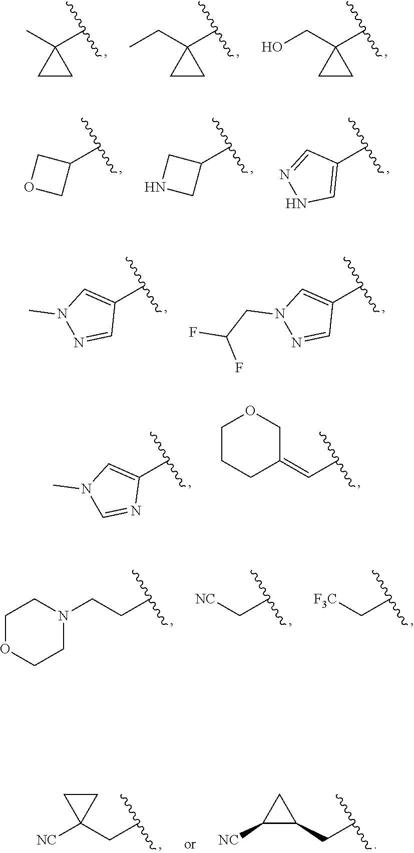

Embodiment A32. The compound of Embodiment A28, or a pharmaceutically acceptable salt or solvate thereof, wherein:
   R^{3a} and R^{3b} are taken together with the intervening atoms to which they are attached to form an optionally substituted 5- to 6-membered heterocycloalkyl.
Embodiment A33. The compound of Embodiment A32, or a pharmaceutically acceptable salt or solvate thereof, wherein:
   R^{3a} and R^{3b} are taken together with the intervening atoms to which they are attached to form

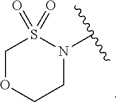 [SMILES: *N1CCOCS1(=O)=O]

Embodiment A34. The compound of any one of Embodiments A1 to A27, or a pharmaceutically acceptable salt or solvate thereof, wherein:
   R³ is —N(R^{3a})₂; and
   each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment A35. The compound of any one of Embodiments A1 to A27, or a pharmaceutically acceptable salt or solvate thereof, wherein:
   R³ is —SO₂R^{3b}; and
   R^{3b} is C₁-C₆ alkyl, C₁-C₆ hydroxyalkyl, or optionally substituted C₃-C₆ cycloalkyl.
Embodiment A36. The compound of Embodiment A35, or a pharmaceutically acceptable salt or solvate thereof, wherein:
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment A37. The compound of Embodiment A35, or a pharmaceutically acceptable salt or solvate thereof, wherein:
   R^{3b} is —CH₃, —CH₂CH₃, —CH₂CH₂CH₃, —CH(CH₃)₂, —CH₂OH, —CH₂CH₂OH, —CH₂CH₂CH₂OH,

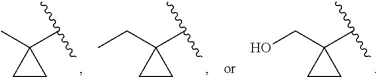

Embodiment A38. The compound of any one of Embodiments A1 to A27, or a pharmaceutically acceptable salt or solvate thereof, wherein:
   R³ is —NR^{3a}C(O)—C(O)R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment A39. The compound of Embodiment A38, or a pharmaceutically acceptable salt or solvate thereof, wherein:
   R^{3a} is hydrogen, —CH₃, —CH₂CH₃, —CH₂CH₂CH₃, or —CH(CH₃)₂; and
   R^{3b} is —CH₃, —CH₂CH₃, —CH₂CH₂CH₃, —CH(CH₃)₂, —CH₂OH, —CH₂CH₂OH, —CH₂CH₂CH₂OH,

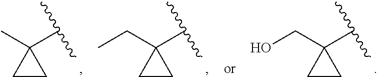

Embodiment A40. The compound of any one of Embodiments A1 to A26, or a pharmaceutically acceptable salt or solvate thereof, wherein:
   R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C═CH, —NHC(O)—C(O)CH₃, —NHSO₂N(CH₃)₂, —NHSO₂NH(CH₂CH₃), —SO₂CH₂CH₂OH, —SO₂HHCH₂CH₂OH,

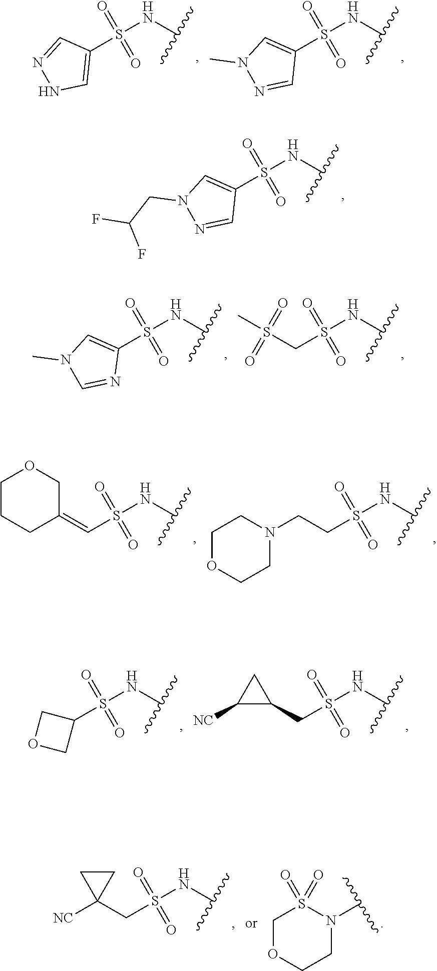

Embodiment A41. The compound of any one of Embodiments A1 to A26, or a pharmaceutically acceptable salt or solvate thereof, wherein:
   R³ is —NHSO₂CH₂CH₃ or —NHSO₂CH₂CH₂OH.
Embodiment A42. The compound of any one of Embodiments A1 to A26, or a pharmaceutically acceptable salt or solvate thereof, wherein:
   R³ is —NHSO₂CH₂CH₂OH.
Embodiment B1. A compound having the structure of Formula (I):

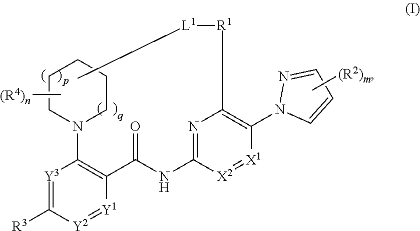

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —OR², —N(R^{2a})₂, —C(O)N(R^{2a})₂, —N(R^{2a})C(O)NR^{2a}, —C(O)R^{2b}, —C(O)OR^{2′}, —S(O)₂N(R^{2a})₂, —S(O)₂R^{2b}, optionally substituted C₁-C₈ alkyl, C₁-C₅ hydroxyalkyl, optionally substituted C₃-C₈ cycloalkyl, or optionally substituted 4- to 8-membered heterocycloalkyl;
   each R^{2a} is independently hydrogen, optionally substituted C₁-C₆ alkyl, or optionally substituted C₃-C₆ cycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl; each R^{2b} is independently optionally substituted C₁-C₆ alkyl;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment B2. The compound of Embodiment B1, or a pharmaceutically acceptable salt or solvate thereof, wherein:
   Y¹ is CR⁵;
   Y² is CR⁵;
   Y³ is CR⁵; and
   each R⁵ is independently hydrogen, halogen, —CN, —OR⁵, —N(R^{5a})₂, or C₁-C₈ alkyl.
Embodiment B3. The compound of Embodiment B1 or Embodiment B2, or a pharmaceutically acceptable salt or solvate thereof, wherein:
   X¹ is CR⁶ or N;
   X² is CR⁶; and
   each R⁶ is independently hydrogen, halogen, —CN, —OR⁵, —N(R^{5a})₂, or C₁-C₈ alkyl.
Embodiment B4. The compound of any one of Embodiments B1 to B3, or a pharmaceutically acceptable salt or solvate thereof, wherein:
   p is 1.
Embodiment B5. The compound of any one of Embodiments B1 to B4, or a pharmaceutically acceptable salt or solvate thereof, wherein:
   q is 1.
Embodiment B6. The compound of Embodiment B1, having the structure of Formula (II):

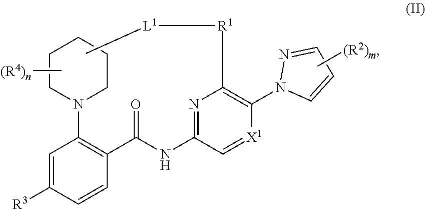

or a pharmaceutically acceptable salt or solvate thereof, wherein:
X¹ is CH or N.
Embodiment B7. The compound of any one of Embodiments B1 to B6, or a pharmaceutically acceptable salt or solvate thereof, wherein:
   R¹ is —NH— or —N(C₁-C₄ alkyl)-.
Embodiment B8. The compound of any one of Embodiments B1 to B7, or a pharmaceutically acceptable salt or solvate thereof, wherein:
   R¹ is —NH—.
Embodiment B9. The compound of any one of Embodiments B1 to B8, or a pharmaceutically acceptable salt or solvate thereof, wherein:
   L¹ is a linker comprising the structure -Lia-L^{1b}-*, where * designates the attachment point to R¹;
      L^{1a} is a bond, —O—, —NH—, —S—, or —SO₂—; and
   L^{1b} is optionally substituted C₄-C₅ alkenylene.
Embodiment B10. The compound of Embodiment B9, or a pharmaceutically acceptable salt or solvate thereof, wherein:
   L^{1a} is a bond or —O—.
Embodiment B11. The compound of Embodiment B9 or B10, or a pharmaceutically acceptable salt or solvate thereof, wherein:
   L^{1a} is a bond.
Embodiment B12. The compound of Embodiment B11, or a pharmaceutically acceptable salt or solvate thereof, wherein:
   L^{1b} is C₅ alkenylene.
Embodiment B13. The compound of Embodiment B9 or B10, or a pharmaceutically acceptable salt or solvate thereof, wherein:
   L^{1a} is —O—.
Embodiment B14. The compound of Embodiment B13, or a pharmaceutically acceptable salt or solvate thereof, wherein:
   L^{1b} is C₄ alkenylene.
Embodiment B15. The compound of Embodiment B9, or a pharmaceutically acceptable salt or solvate thereof, wherein:
   L^{1a} is a bond; and L^{1b} is C₅ alkenylene;
   or L^{1a} is —O—; and L^{1b} is C₄ alkenylene.
Embodiment B16. The compound of Embodiment B9, or a pharmaceutically acceptable salt or solvate thereof, wherein L¹ is —CH₂CH═CHCH₂CH₂—*, —CH₂CH₂CH═CHCH₂—*, or —OCH₂CH═CHCH₂—*, where * designates the attachment point to R¹.
Embodiment B17. The compound of Embodiment B1, having the structure of Formula (IIIa) or (IIIb):

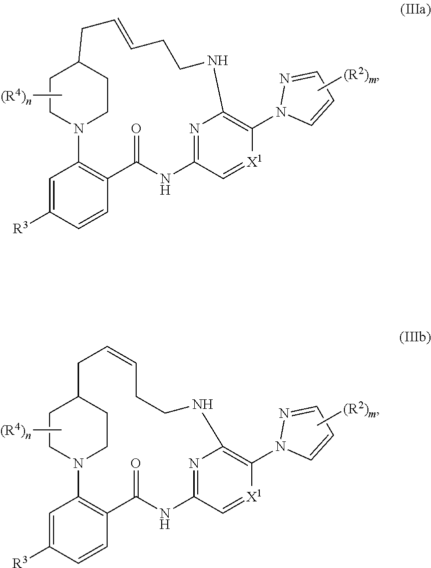

or a pharmaceutically acceptable salt or solvate thereof, wherein:
X¹ is CH or N.
Embodiment B18. The compound of any one of Embodiments B1 to B8, or a pharmaceutically acceptable salt or solvate thereof, wherein:
   L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.
Embodiment B19. The compound of Embodiment B18, or a pharmaceutically acceptable salt or solvate thereof, wherein:
   L¹ is optionally substituted C₄-C₅ heteroalkylene; wherein the terminal atoms of the heteroalkylene are C, and the heteroalkylene comprises 1 heteroatom selected from N, O, and S.
Embodiment B20. The compound of Embodiment B18 or B19, or a pharmaceutically acceptable salt or solvate thereof, wherein:
   L¹ is optionally substituted C₅ heteroalkylene; wherein the terminal atoms of the heteroalkylene are C, and the heteroalkylene comprises 1 O atom.
Embodiment B21. The compound of Embodiment B18, or a pharmaceutically acceptable salt or solvate thereof, wherein L¹ is —CH₂OCH₂CH₂CH₂—* or —CH₂CH₂OCH₂CH₂—*, where * designates the attachment point to R¹.
Embodiment B22. The compound of Embodiment B1, having the structure of Formula (VIa), (VIb), (VIIa), or (VIIb):

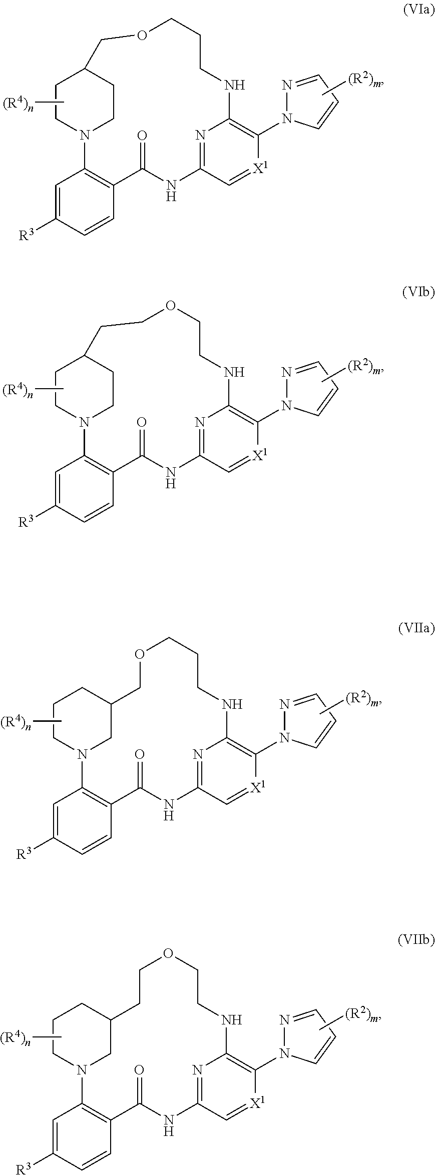

or a pharmaceutically acceptable salt or solvate thereof, wherein:
X¹ is CH or N.
Embodiment B23. The compound of any one of Embodiments B1 to B22, or a pharmaceutically acceptable salt or solvate thereof, wherein:
   X¹ is CH.
Embodiment B24. The compound of any one of Embodiments B1 to B22, or a pharmaceutically acceptable salt or solvate thereof, wherein:
   X¹ is N.
Embodiment B25. The compound of any one of Embodiments B1 to B24, or a pharmaceutically acceptable salt or solvate thereof, wherein:
   each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl;
   each R^{2a} is independently hydrogen or C₁-C₆ alkyl; wherein the alkyl is optionally substituted with 1, 2, or 3 groups independently selected from halogen, —OH, and —CN;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl;
   and
   each R^{2b} is independently C₁-C₆ alkyl.
Embodiment B26. The compound of any one of Embodiments B1 to B25, or a pharmaceutically acceptable salt or solvate thereof, wherein:
   each R² is independently —F, —Cl, —CN, —OR^{2a}, —C(O)N(R^{2a})₂, —S(O)₂R^{2b}, or C₁-C₄ hydroxyalkyl;
   each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH;
   or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and
   each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment B27. The compound of any one of Embodiments B1 to B26, or a pharmaceutically acceptable salt or solvate thereof, wherein:
   each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

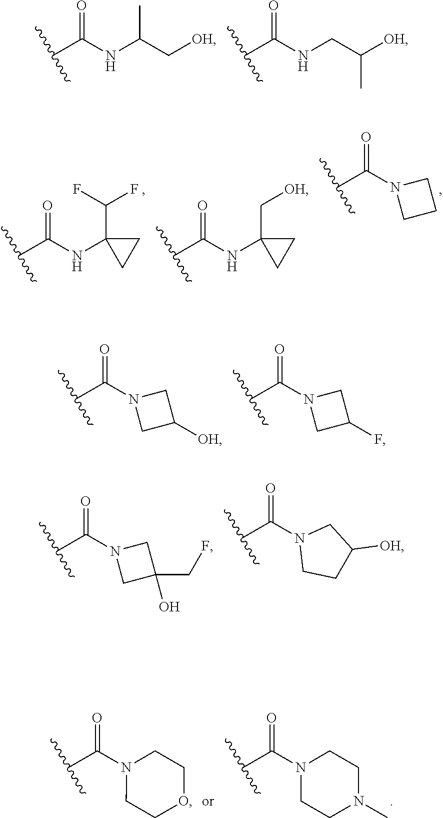

Embodiment B28. The compound of any one of Embodiments B1 to B27, or a pharmaceutically acceptable salt or solvate thereof, wherein:
   m is 1.
Embodiment B29. The compound of any one of Embodiments B1 to B28, or a pharmaceutically acceptable salt or solvate thereof, wherein:
   each R⁴ is independently halogen, —CN, C₁-C₆ alkyl, or C₁-C₆ fluoroalkyl.
Embodiment B30. The compound of any one of Embodiments B1 to B29, or a pharmaceutically acceptable salt or solvate thereof, wherein:
   each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment B31. The compound of any one of Embodiments B1 to B30, or a pharmaceutically acceptable salt or solvate thereof, wherein:
   each R⁴ is independently —CN or —CH₃.
Embodiment B32. The compound of any one of Embodiments B1 to B31, or a pharmaceutically acceptable salt or solvate thereof, wherein:
   n is 1.
Embodiment B33. The compound of any one of Embodiments B1 to B32, or a pharmaceutically acceptable salt or solvate thereof, wherein:
   R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b}; each R^{3a} is independently hydrogen or C₁-C₆ alkyl;
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl.
Embodiment B34. The compound of any one of Embodiments B1 to B33, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
   or R^{3a} and R^{3b} are taken together with the intervening atoms to which they are attached to form an optionally substituted 5- to 6-membered heterocycloalkyl.
Embodiment B35. The compound of Embodiment B34, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment B36. The compound of Embodiment B35, or a pharmaceutically acceptable salt or solvate thereof, wherein:
   R^{3a} is hydrogen or C₁-C₃ alkyl; and
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment B37. The compound of Embodiment B34, or a pharmaceutically acceptable salt or solvate thereof, wherein:
   R^{3a} is hydrogen, —CH₃, or —CH₂CH₃; and
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH,

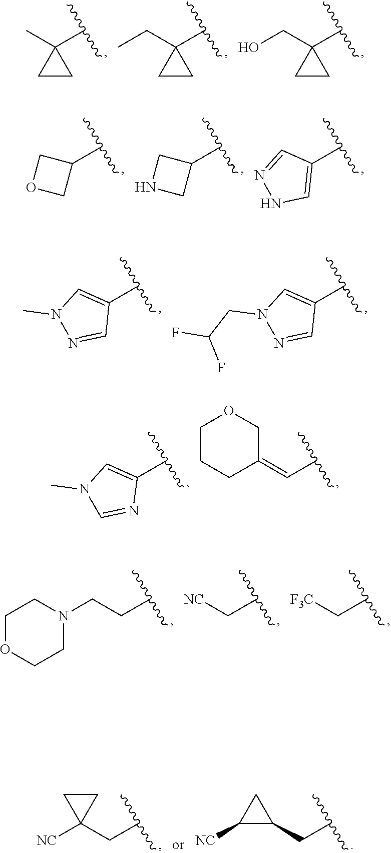

Embodiment B38. The compound of Embodiment B34, or a pharmaceutically acceptable salt or solvate thereof, wherein:
   R^{3a} and R^{3b} are taken together with the intervening atoms to which they are attached to form an optionally substituted 5- to 6-membered heterocycloalkyl.
Embodiment B39. The compound of Embodiment B38, or a pharmaceutically acceptable salt or solvate thereof, wherein:
   R^{3a} and R^{3b} are taken together with the intervening atoms to which they are attached to form

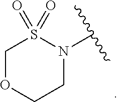 [SMILES: *N1CCOCS1(=O)=O]

Embodiment B40. The compound of any one of Embodiments B1 to B33, or a pharmaceutically acceptable salt or solvate thereof, wherein:
   R³ is —N(R^{3a})₂; and
   each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment B41. The compound of any one of Embodiments B1 to B33, or a pharmaceutically acceptable salt or solvate thereof, wherein:
   R³ is —SO₂R^{3b}; and
   R^{3b} is C₁-C₆ alkyl, C₁-C₆ hydroxyalkyl, or optionally substituted C₃-C₆ cycloalkyl.
Embodiment B42. The compound of Embodiment B41, or a pharmaceutically acceptable salt or solvate thereof, wherein:
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment B43. The compound of Embodiment B41, or a pharmaceutically acceptable salt or solvate thereof, wherein:
   R^{3b} is —CH₃, —CH₂CH₃, —CH₂CH₂CH₃, —CH(CH₃)₂, —CH₂OH, —CH₂CH₂OH, —CH₂CH₂CH₂OH,

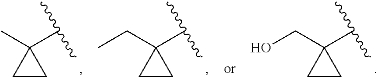

Embodiment B44. The compound of any one of Embodiments B1 to B33, or a pharmaceutically acceptable salt or solvate thereof, wherein:
   R³ is —NR^{3a}C(O)—C(O)R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment B45. The compound of Embodiment B44, or a pharmaceutically acceptable salt or solvate thereof, wherein:
   R^{3a} is hydrogen, —CH₃, —CH₂CH₃, —CH₂CH₂CH₃, or —CH(CH₃)₂; and
   R^{3b} is —CH₃, —CH₂CH₃, —CH₂CH₂CH₃, —CH(CH₃)₂, —CH₂OH, —CH₂CH₂OH, —CH₂CH₂CH₂OH,

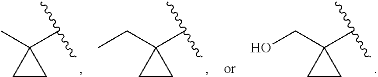

Embodiment B46. The compound of any one of Embodiments B1 to B32, or a pharmaceutically acceptable salt or solvate thereof, wherein:
   R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHC(O)—C(O)CH₃, —NHSO₂N(CH₃)₂, —NHSO₂NH(CH₂CH₃), —SO₂CH₂CH₂OH, —SO₂NHCH₂CH₂OH,

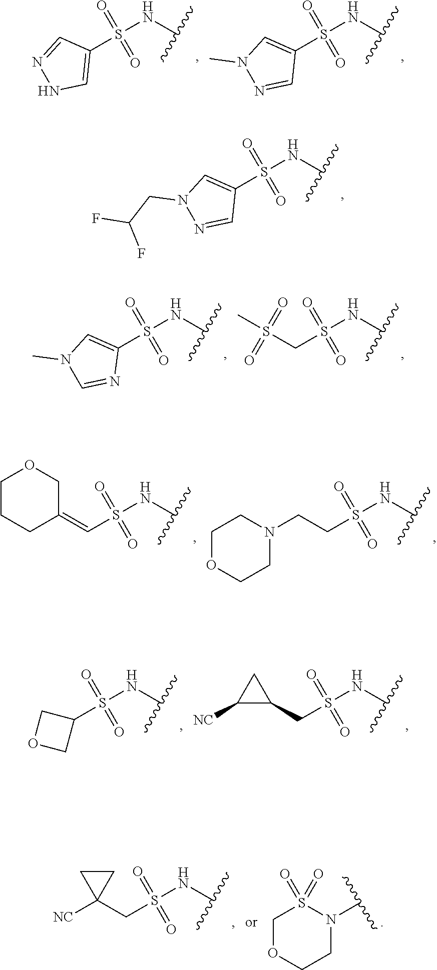

Embodiment B47. The compound of any one of Embodiments B1 to B32, or a pharmaceutically acceptable salt or solvate thereof, wherein:
   R³ is —NHSO₂CH₂CH₃ or —NHSO₂CH₂CH₂OH.
Embodiment C1. A compound having the structure of Formula (I):

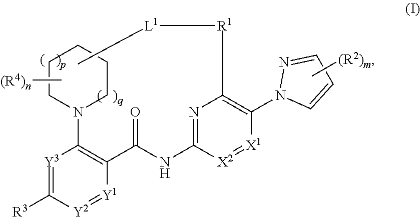

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -Lia-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —OR^{2a}, —N(R^{2a})₂, —C(O)N(R^{2a})₂, —N(R^{2a})C(O)NR^{2a}, —C(O)R^{2b}, —C(O)OR^{2a}, —S(O)₂N(R^{2a})₂, —S(O)₂R^{2b}, optionally substituted C₁-C₈ alkyl, C₁-C₅ hydroxyalkyl, optionally substituted C₃-C₈ cycloalkyl, or optionally substituted 4- to 8-membered heterocycloalkyl;
   each R^{2a} is independently hydrogen, optionally substituted C₁-C₆ alkyl, or optionally substituted C₃-C₆ cycloalkyl;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b}; each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6′}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment C2. The compound of Embodiment C1, or a pharmaceutically acceptable salt or solvate thereof, wherein:
   Y¹ is CR⁵;
   Y² is CR⁵;
   Y³ is CR⁵; and
   each R⁵ is independently hydrogen, halogen, —CN, —OR^{5a}, —N(R^{5a})₂, or C₁-C₈ alkyl.
Embodiment C3. The compound of Embodiment C1 or Embodiment C2, or a pharmaceutically acceptable salt or solvate thereof, wherein:
   X¹ is CR⁶ or N;
   X² is CR⁶; and
   each R⁶ is independently hydrogen, halogen, —CN, —OR^{5a}, —N(R^{5a})₂, or C₁-C₈ alkyl.
Embodiment C4. The compound of any one of Embodiments C1 to C3, or a pharmaceutically acceptable salt or solvate thereof, wherein:
   p is 1.
Embodiment C5. The compound of any one of Embodiments C1 to C4, or a pharmaceutically acceptable salt or solvate thereof, wherein:
   q is 1.
Embodiment C6. The compound of Embodiment C1, having the structure of Formula (II):

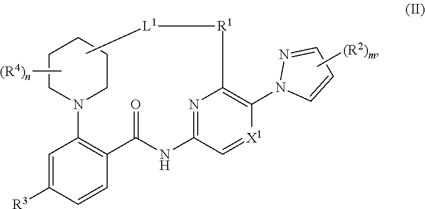

or a pharmaceutically acceptable salt or solvate thereof, wherein:
X¹ is CH or N.
Embodiment C7. The compound of any one of Embodiments C1 to C6, or a pharmaceutically acceptable salt or solvate thereof, wherein:
   R¹ is —NH— or —N(C₁-C₄ alkyl)-.
Embodiment C8. The compound of any one of Embodiments C1 to C7, or a pharmaceutically acceptable salt or solvate thereof, wherein:
   R¹ is —NH—.
Embodiment C9. The compound of any one of Embodiments C1 to C8, or a pharmaceutically acceptable salt or solvate thereof, wherein:
   L¹ is a linker comprising the structure -L^{1a}-L^{1b}-*, where * designates the attachment point to R¹;
      L^{1a} is a bond, —O—, —NH—, —S—, or —SO₂—; and
      L^{1b} is optionally substituted C₄-C₅ alkenylene.
Embodiment C10. The compound of Embodiment C9, or a pharmaceutically acceptable salt or solvate thereof, wherein:
   L^{1a} is a bond or —O—.
Embodiment C11. The compound of Embodiment C9 or Embodiment C10, or a pharmaceutically acceptable salt or solvate thereof, wherein:
   L^{1a} is a bond.
Embodiment C12. The compound of Embodiment C11, or a pharmaceutically acceptable salt or solvate thereof, wherein:
   L^{1b} is C₅ alkenylene.
Embodiment C13. The compound of Embodiment C9 or Embodiment C10, or a pharmaceutically acceptable salt or solvate thereof, wherein:
   L^{1a} is —O—.
Embodiment C14. The compound of Embodiment C13, or a pharmaceutically acceptable salt or solvate thereof, wherein:
   L^{1b} is C₄ alkenylene.
Embodiment C15. The compound of Embodiment C9, or a pharmaceutically acceptable salt or solvate thereof, wherein:
   L^{1a} is a bond; and L^{1b} is C₅ alkenylene;
   or L^{1a} is —O—; and L^{1b} is C₄ alkenylene.
Embodiment C16. The compound of Embodiment C9, or a pharmaceutically acceptable salt or solvate thereof, wherein L¹ is —CH₂CH═CHCH₂CH₂—*, —CH₂CH₂CH═CHCH₂—*, or —OCH₂CH═CHCH₂—*, where * designates the attachment point to R¹.
Embodiment C17. The compound of Embodiment A1, having the structure of Formula (IIIa), (IIIb), (IVa), or (IVb):

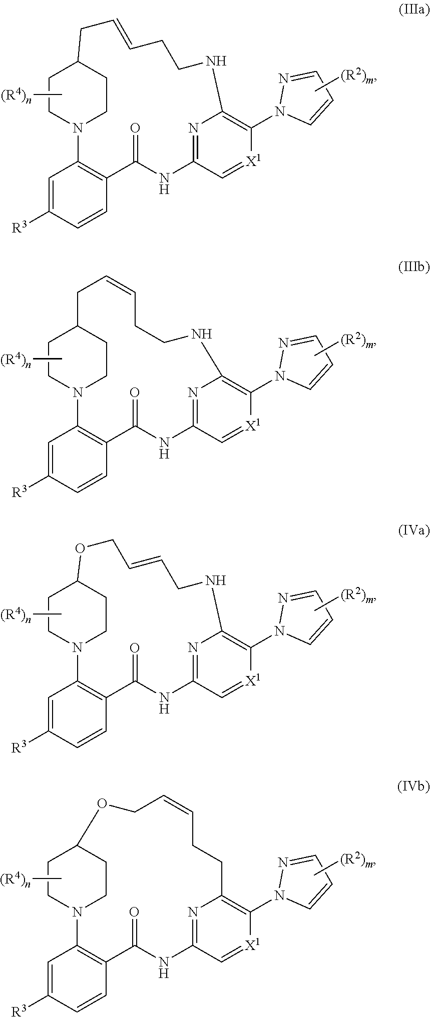

or a pharmaceutically acceptable salt or solvate thereof, wherein:
X¹ is CH or N.
Embodiment C18. The compound of any one of Embodiments C1 to C8, or a pharmaceutically acceptable salt or solvate thereof, wherein:
   L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.
Embodiment C19. The compound of Embodiment C18, or a pharmaceutically acceptable salt or solvate thereof, wherein:
   L¹ is optionally substituted C₄-C₅ heteroalkylene; wherein the terminal atoms of the heteroalkylene are C, and the heteroalkylene comprises 1 heteroatom selected from N, O, and S.
Embodiment C20. The compound of Embodiment C18 or Embodiment C19, or a pharmaceutically acceptable salt or solvate thereof, wherein:
   L¹ is optionally substituted C₅ heteroalkylene; wherein the terminal atoms of the heteroalkylene are C, and the heteroalkylene comprises 1 O atom.
Embodiment C21. The compound of Embodiment C18, or a pharmaceutically acceptable salt or solvate thereof, wherein L¹ is —CH₂OCH₂CH₂CH₂—* or —CH₂CH₂OCH₂CH₂—*, where * designates the attachment point to R¹.
Embodiment C22. The compound of Embodiment A1, having the structure of Formula (VIa), (VIb), (VIIa), or (VIIb):

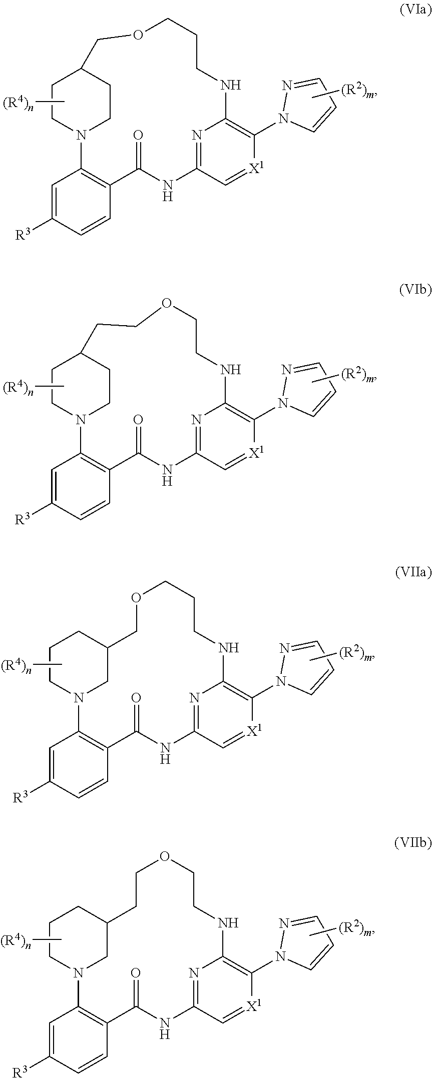

or a pharmaceutically acceptable salt or solvate thereof, wherein:
X¹ is CH or N.
Embodiment C23. The compound of any one of Embodiments C1 to C22, or a pharmaceutically acceptable salt or solvate thereof, wherein:
   X¹ is CH.
Embodiment C24. The compound of any one of Embodiments C1 to C22, or a pharmaceutically acceptable salt or solvate thereof, wherein:
   X¹ is N.
Embodiment C25. The compound of any one of Embodiments C1 to C24, or a pharmaceutically acceptable salt or solvate thereof, wherein:
   each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl;
   each R^{2a} is independently hydrogen or C₁-C₆ alkyl; wherein the alkyl is optionally substituted with 1, 2, or 3 groups independently selected from halogen, —OH, and —CN;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl;
   and
   each R^{2b} is independently C₁-C₆ alkyl.
Embodiment C26. The compound of any one of Embodiments C1 to C25, or a pharmaceutically acceptable salt or solvate thereof, wherein:
   each R² is independently —F, —Cl, —CN, —OR^{2a}, —C(O)N(R^{2a})₂, —S(O)₂R²¹, or C₁-C₄ hydroxyalkyl;
   each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH;
   or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and
   each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment C27. The compound of any one of Embodiments C1 to C26, or a pharmaceutically acceptable salt or solvate thereof, wherein:
   each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

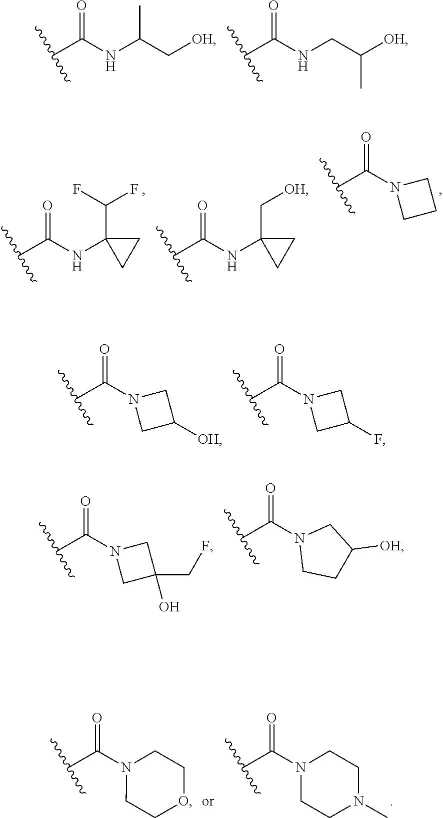

Embodiment C28. The compound of any one of Embodiments C1 to C27, or a pharmaceutically acceptable salt or solvate thereof, wherein:
   m is 1.
Embodiment C29. The compound of any one of Embodiments C1 to C28, or a pharmaceutically acceptable salt or solvate thereof, wherein:
   each R⁴ is independently halogen, —CN, C₁-C₆ alkyl, or C₁-C₆ fluoroalkyl.
Embodiment C30. The compound of any one of Embodiments C1 to C29, or a pharmaceutically acceptable salt or solvate thereof, wherein:
   each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment C31. The compound of any one of Embodiments C1 to C30, or a pharmaceutically acceptable salt or solvate thereof, wherein:
   each R⁴ is independently —CN or —CH₃.
Embodiment C32. The compound of any one of Embodiments C1 to C31, or a pharmaceutically acceptable salt or solvate thereof, wherein:
   n is 1.
Embodiment C33. The compound of any one of Embodiments C1 to C32, or a pharmaceutically acceptable salt or solvate thereof, wherein:
   R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or C₁-C₆ alkyl;
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl.
Embodiment C34. The compound of any one of Embodiments C1 to C33, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
   or R^{3a} and R^{3b} are taken together with the intervening atoms to which they are attached to form an optionally substituted 5- to 6-membered heterocycloalkyl.
Embodiment C35. The compound of Embodiment C34, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment C36. The compound of Embodiment C35, or a pharmaceutically acceptable salt or solvate thereof, wherein:
   R^{3a} is hydrogen or C₁-C₃ alkyl; and
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment C37. The compound of Embodiment C34, or a pharmaceutically acceptable salt or solvate thereof, wherein:
   R^{3a} is hydrogen, —CH₃, or —CH₂CH₃; and
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH, —CH₂CH₂CH₂CH₂C≡CH,

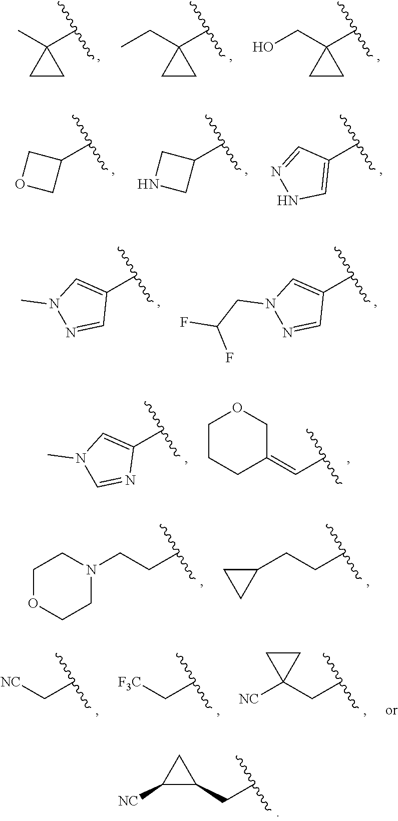

Embodiment C38. The compound of Embodiment C34, or a pharmaceutically acceptable salt or solvate thereof, wherein:
   R^{3a} and R^{3b} are taken together with the intervening atoms to which they are attached to form an optionally substituted 5- to 6-membered heterocycloalkyl.
Embodiment C39. The compound of Embodiment C38, or a pharmaceutically acceptable salt or solvate thereof, wherein:
   R^{3a} and R^{3b} are taken together with the intervening atoms to which they are attached to form

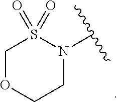 [SMILES: *N1CCOCS1(=O)=O]

Embodiment C40. The compound of any one of Embodiments C1 to C33, or a pharmaceutically acceptable salt or solvate thereof, wherein:
   R³ is —N(R^{3a})₂; and
   each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment C41. The compound of any one of Embodiments C1 to C33, or a pharmaceutically acceptable salt or solvate thereof, wherein:
   R³ is —SO₂R^{3b}; and
   R^{3b} is C₁-C₆ alkyl, C₁-C₆ hydroxyalkyl, or optionally substituted C₃-C₆ cycloalkyl.
Embodiment C42. The compound of Embodiment C41, or a pharmaceutically acceptable salt or solvate thereof, wherein:
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment C43. The compound of Embodiment C41, or a pharmaceutically acceptable salt or solvate thereof, wherein:
   R^{3b} is —CH₃, —CH₂CH₃, —CH₂CH₂CH₃, —CH(CH₃)₂, —CH₂OH, —CH₂CH₂OH, —CH₂CH₂CH₂OH,

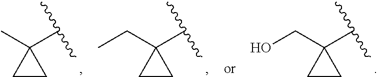

Embodiment C44. The compound of any one of Embodiments C1 to C33, or a pharmaceutically acceptable salt or solvate thereof, wherein:
   R³ is —NR^{3a}C(O)—C(O)R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment C45. The compound of Embodiment C44, or a pharmaceutically acceptable salt or solvate thereof, wherein:
   R^{3a} is hydrogen, —CH₃, —CH₂CH₃, —CH₂CH₂CH₂, or —CH(CH₃)₂; and
   R^{3b} is —CH₃, —CH₂CH₃, —CH₂CH₂CH₃, —CH(CH₃)₂, —CH₂OH, —CH₂CH₂OH, —CH₂CH₂CH₂OH,

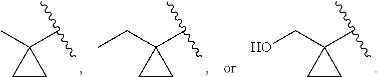

Embodiment C46. The compound of any one of Embodiments C1 to C32, or a pharmaceutically acceptable salt or solvate thereof, wherein:
   R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂NHCH₂CH₂OH,

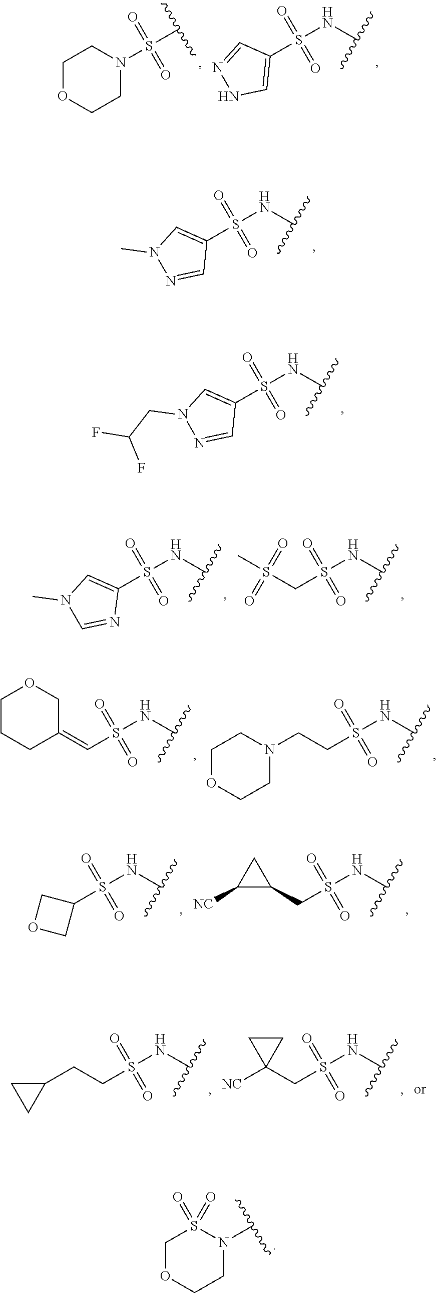

Embodiment C47. The compound of any one of Embodiments C1 to C32, or a pharmaceutically acceptable salt or solvate thereof, wherein:
   R³ is —NHSO₂CH₂CH₃ or —NHSO₂CH₂CH₂OH.
Embodiment C48. The compound of any one of Embodiments C1 to C32, or a pharmaceutically acceptable salt or solvate thereof, wherein:
   R³ is —NHSO₂CH₂CH₂OH.
Embodiment D1. A compound having the structure of Formula (I):

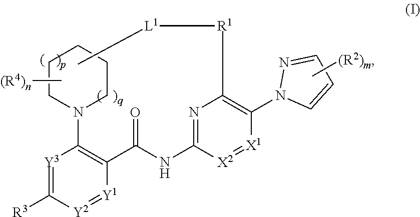

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₅ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁—C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6′}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment D2. The compound of Embodiment D1, or a pharmaceutically acceptable salt or solvate thereof, wherein:
   Y¹ is CR⁵;
   Y² is CR⁵;
   Y³ is CR⁵; and
   each R⁵ is independently hydrogen, halogen, —CN, —OR^{5a}, —N(R^{5a})₂, or C₁-C₈ alkyl.
Embodiment D3. The compound of Embodiment D1 or Embodiment D2, or a pharmaceutically acceptable salt or solvate thereof, wherein:
   X¹ is CR⁶ or N;
   X² is CR⁶; and
   each R⁶ is independently hydrogen, halogen, —CN, —OR^{5a}, —N(R^{5a})₂, or C₁-C₈ alkyl.
Embodiment D4. The compound of any one of Embodiments D1 to D3, or a pharmaceutically acceptable salt or solvate thereof, wherein:
   p is 1.
Embodiment D5. The compound of any one of Embodiments D1 to D4, or a pharmaceutically acceptable salt or solvate thereof, wherein:
   q is 1.
Embodiment D6. The compound of Embodiment D1, having the structure of Formula (II):

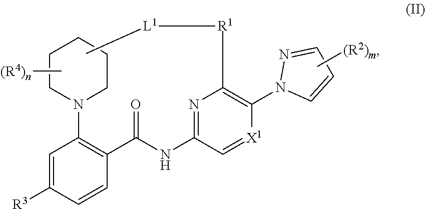

or a pharmaceutically acceptable salt or solvate thereof, wherein:
X¹ is CH or N.
Embodiment D7. The compound of any one of Embodiments D1 to D6, or a pharmaceutically acceptable salt or solvate thereof, wherein:
   R¹ is —NH— or —N(C₁-C₄ alkyl)-.
Embodiment D8. The compound of any one of Embodiments D1 to D7, or a pharmaceutically acceptable salt or solvate thereof, wherein:
   R¹ is —NH—.
Embodiment D9. The compound of any one of Embodiments D1 to D8, or a pharmaceutically acceptable salt or solvate thereof, wherein:
   L¹ is a linker comprising the structure -L^{1a}-L^{1b}-*, where * designates the attachment point to R¹;
      L^{1a} is a bond, —O—, —NH—, —S—, or —SO₂—; and
      L^{1b} is optionally substituted C₄-C₅ alkenylene.
Embodiment D10. The compound of Embodiment D9, or a pharmaceutically acceptable salt or solvate thereof, wherein:
   L^{1a} is a bond or —O—.
Embodiment D11. The compound of Embodiment D9 or D10, or a pharmaceutically acceptable salt or solvate thereof, wherein:
   L^{1a} is a bond.
Embodiment D12. The compound of Embodiment D11, or a pharmaceutically acceptable salt or solvate thereof, wherein:
   L^{1b} is C₅ alkenylene.
Embodiment D13. The compound of Embodiment D9 or D10, or a pharmaceutically acceptable salt or solvate thereof, wherein:
   L^{1a} is —O—.
Embodiment D14. The compound of Embodiment D13, or a pharmaceutically acceptable salt or solvate thereof, wherein:
   L^{1b} is C₄ alkenylene.
Embodiment D15. The compound of Embodiment D9, or a pharmaceutically acceptable salt or solvate thereof, wherein:
   L^{1a} is a bond; and L^{1b} is C₅ alkenylene;
   or L^{1a} is —O—; and L^{1b} is C₄ alkenylene.
Embodiment D16. The compound of Embodiment D9, or a pharmaceutically acceptable salt or solvate thereof, wherein L¹ is —CH₂CH═CHCH₂CH₂—*, —CH₂CH₂CH═CHCH₂—*, or —OCH₂CH═CHCH₂—*, where * designates the attachment point to RU.
Embodiment D17. The compound of Embodiment D1, having the structure of Formula (IIIa), (IIIb), (IVa), or (IVb):

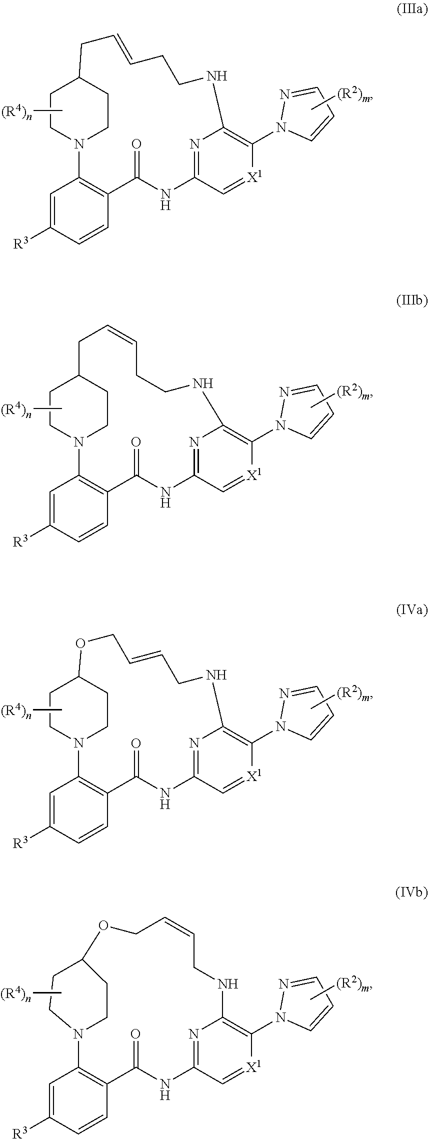

or a pharmaceutically acceptable salt or solvate thereof, wherein:
X¹ is CH or N.
Embodiment D18. The compound of any one of Embodiments D1 to D8, or a pharmaceutically acceptable salt or solvate thereof, wherein:
   L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.
Embodiment D19. The compound of Embodiment D18, or a pharmaceutically acceptable salt or solvate thereof, wherein:
   L¹ is optionally substituted C₄-C₅ heteroalkylene; wherein the terminal atoms of the heteroalkylene are C, and the heteroalkylene comprises 1 heteroatom selected from N, O, and S.
Embodiment D20. The compound of Embodiment D18 or D19, or a pharmaceutically acceptable salt or solvate thereof, wherein:
   L¹ is optionally substituted C₅ heteroalkylene; wherein the terminal atoms of the heteroalkylene are C, and the heteroalkylene comprises 1 O atom.
Embodiment D21. The compound of Embodiment D18, or a pharmaceutically acceptable salt or solvate thereof, wherein L¹ is —CH₂OCH₂CH₂CH₂—* or —CH₂CH₂OCH₂CH₂—*, where * designates the attachment point to R¹.
Embodiment D22. The compound of Embodiment D1, having the structure of Formula (VIa), (VIb), (VIIa), or (VIIb):

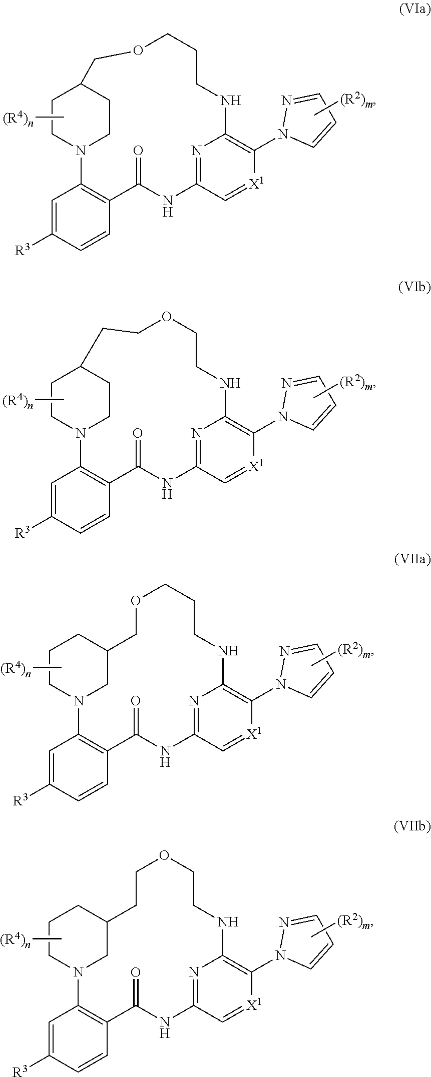

or a pharmaceutically acceptable salt or solvate thereof, wherein:
X¹ is CH or N.
Embodiment D23. The compound of any one of Embodiments D1 to D22, or a pharmaceutically acceptable salt or solvate thereof, wherein:
   X¹ is CH.
Embodiment D24. The compound of any one of Embodiments D1 to D22, or a pharmaceutically acceptable salt or solvate thereof, wherein:
   X¹ is N.
Embodiment D25. The compound of any one of Embodiments D1 to D24, or a pharmaceutically acceptable salt or solvate thereof, wherein:
   each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl;
   each R^{2a} is independently hydrogen or C₁-C₆ alkyl; wherein the alkyl is optionally substituted with 1, 2, or 3 groups independently selected from halogen, —OH, and —CN;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl; and
   each R^{2b} is independently C₁-C₆ alkyl.
Embodiment D26. The compound of any one of Embodiments D1 to D25, or a pharmaceutically acceptable salt or solvate thereof, wherein:
   each R² is independently —F, —Cl, —CN, —OR^{2a}, —C(O)N(R^{2a})₂, —S(O)₂R^{2b}, or C₁-C₄ hydroxyalkyl;
   each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH;
   or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and
   each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment D27. The compound of any one of Embodiments D1 to D24, or a pharmaceutically acceptable salt or solvate thereof, wherein:
each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₂, —CH₂OH,

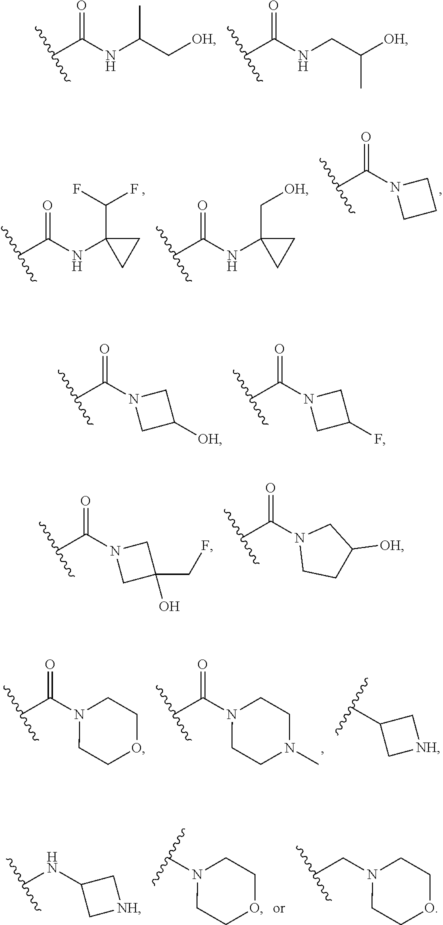

Embodiment D28. The compound of any one of Embodiments D1 to D27, or a pharmaceutically acceptable salt or solvate thereof, wherein:
   m is 1.
Embodiment D29. The compound of any one of Embodiments D1 to D28, or a pharmaceutically acceptable salt or solvate thereof, wherein:
   each R⁴ is independently halogen, —CN, C₁-C₆ alkyl, or C₁-C₆ fluoroalkyl.
Embodiment D30. The compound of any one of Embodiments D1 to D29, or a pharmaceutically acceptable salt or solvate thereof, wherein:
   each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment D31. The compound of any one of Embodiments D1 to D30, or a pharmaceutically acceptable salt or solvate thereof, wherein:
   each R⁴ is independently —CN or —CH₃.
Embodiment D32. The compound of any one of Embodiments D1 to D31, or a pharmaceutically acceptable salt or solvate thereof, wherein:
   n is 1.
Embodiment D33. The compound of any one of Embodiments D1 to D32, or a pharmaceutically acceptable salt or solvate thereof, wherein:
   R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
      each R^{3a} is independently hydrogen or C₁-C₆ alkyl;
      R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl;
      or R^{3a} and R^{3b} are taken together with the intervening atoms to which they are attached to form an optionally substituted 4- to 8-membered heterocycloalkyl.
Embodiment D34. The compound of any one of Embodiments D1 to D33, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
   or R^{3a} and R^{3b} are taken together with the intervening atoms to which they are attached to form an optionally substituted 5- to 6-membered heterocycloalkyl.
Embodiment D35. The compound of Embodiment D34, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment D36. The compound of Embodiment D35, or a pharmaceutically acceptable salt or solvate thereof, wherein:
   R^{3a} is hydrogen or C₁-C₃ alkyl; and
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment D37. The compound of Embodiment D34, or a pharmaceutically acceptable salt or solvate thereof, wherein:
   R^{3a} is hydrogen, —CH₃, or —CH₂CH₃; and
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH, —CH₂CH₂CH₂CH₂C≡CH,

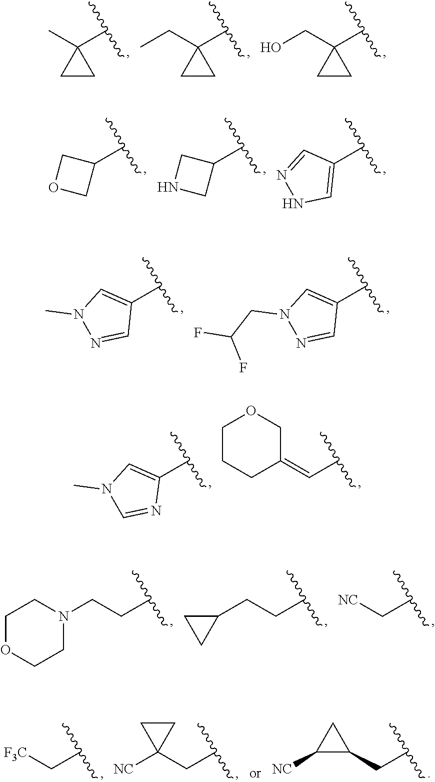

Embodiment D38. The compound of Embodiment D34, or a pharmaceutically acceptable salt or solvate thereof, wherein:
   R^{3a} and R^{3b} are taken together with the intervening atoms to which they are attached to form an optionally substituted 5- to 6-membered heterocycloalkyl.
Embodiment D39. The compound of Embodiment D38, or a pharmaceutically acceptable salt or solvate thereof, wherein:
   R^{3a} and R^{3b} are taken together with the intervening atoms to which they are attached to form

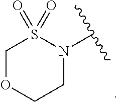 [SMILES: *N1CCOCS1(=O)=O]

Embodiment D40. The compound of any one of Embodiments D1 to D33, or a pharmaceutically acceptable salt or solvate thereof, wherein:
   R³ is —N(R^{3a})₂; and
   each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment D41. The compound of any one of Embodiments D1 to D33, or a pharmaceutically acceptable salt or solvate thereof, wherein:
   R³ is —SO₂R^{3b}; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic 5-membered heteroaryl.
Embodiment D42. The compound of Embodiment D41, or a pharmaceutically acceptable salt or solvate thereof, wherein:
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment D43. The compound of Embodiment D41, or a pharmaceutically acceptable salt or solvate thereof, wherein:
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂,

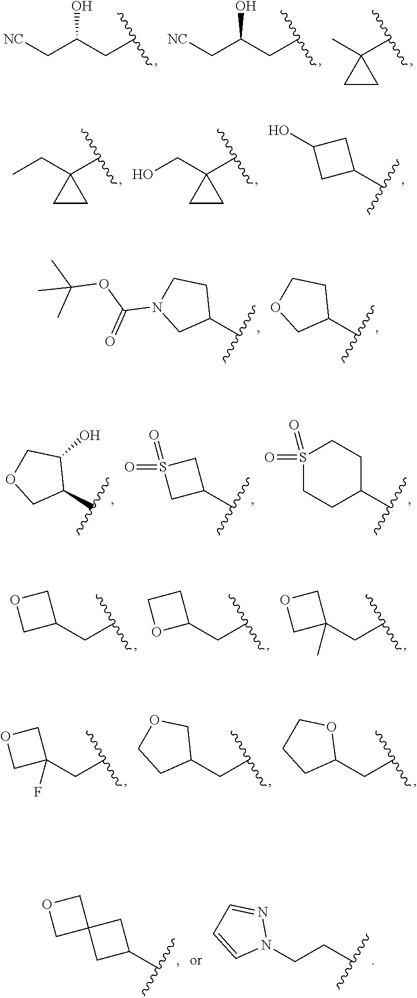

Embodiment D44. The compound of any one of Embodiments D1 to D33, or a pharmaceutically acceptable salt or solvate thereof, wherein:
   R³ is —NR^{3a}C(O)—C(O)R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment D45. The compound of Embodiment D44, or a pharmaceutically acceptable salt or solvate thereof, wherein:
   R^{3a} is hydrogen, —CH₃, —CH₂CH₃, —CH₂CH₂CH₃, or —CH(CH₃)₂; and
   R^{3b} is —CH₃, —CH₂CH₃, —CH₂CH₂CH₃, —CH(CH₃)₂, —CH₂OH, —CH₂CH₂OH, —CH₂CH₂CH₂OH,

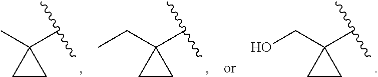

Embodiment D46. The compound of any one of Embodiments D1 to D32, or a pharmaceutically acceptable salt or solvate thereof, wherein:
   R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂CH₂CH₂OCH₃, —SO₂CH₂CH₂CH₂OH, —SO₂CH₂C(CH₃)₂OH, —SO₂CH₂CH₂C(CH₃)₂OH, —SO₂CH(CH₃)CH₂OH, —SO₂CH₂CH₂SO₂CH₃, —SO₂CH₂CH₂OCH₂CN, —SO₂CH₂CH(CH₃)OH, —SO₂CH₂CH₂CN, —SO₂CH(CH₃)CN, —SO₂CH₂CH₂C(═CH₂)CN, —SO₂CH₂C(CH₃)(OH)CH₂OH, —SO₂CH₂C(═O)NH₂, —SO₂NHCH₂CH₂OH,

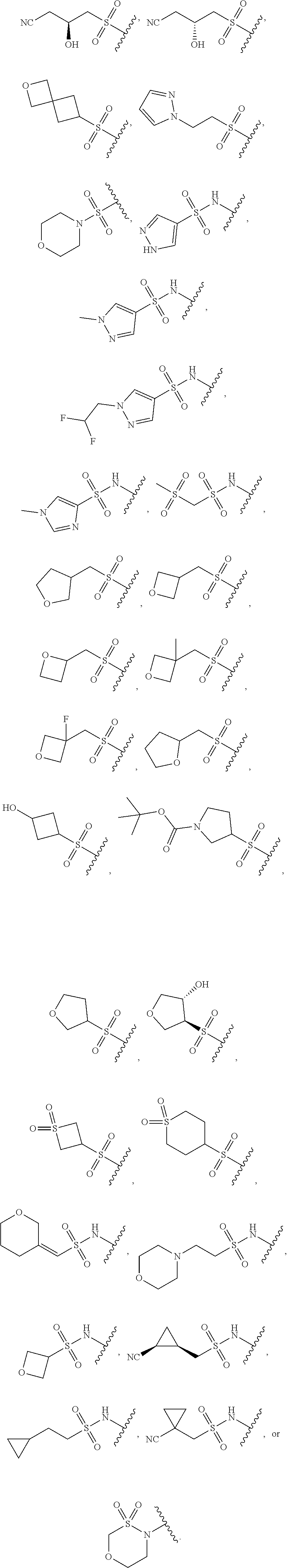

Embodiment D47. The compound of any one of Embodiments D1 to D32, or a pharmaceutically acceptable salt or solvate thereof, wherein:
   R³ is —NHSO₂CH₂CH₃, —NHSO₂CH₂CH₂OH, —SO₂CH₂CH₃, or —SO₂CH₂CH₂OH.
Embodiment D48. The compound of any one of Embodiments D1 to D32, or a pharmaceutically acceptable salt or solvate thereof, wherein:
   R³ is —NHSO₂CH₂CH₃ or —NHSO₂CH₂CH₂OH.
Embodiment E1. A compound having the structure of Formula (I-0):

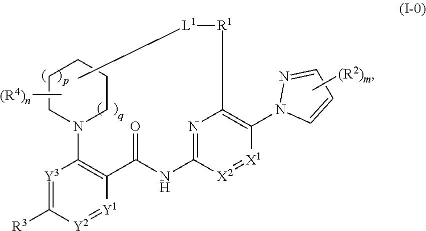

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR⁵, —N(R^{5a})₂, —C(O)N(R^{5a})₂, —N(R^{5a})C(O)NR^{5a}, —C(O)R^{5b}, —C(O)OR⁵, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6b}, —C(O)OR^{6a}, —S(O)₂N(R^{6a})₂, —S(O)₂R^{6b}, optionally substituted C₁-C₅ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment E2. A compound having the structure of Formula (I):

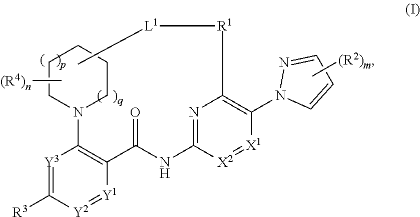

or a pharmaceutically acceptable salt or solvate thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
   L^{1a} is a bond, —O—, —NH—, —S—, or —SO₂—;
   L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
   each R^{2a} is independently hydrogen, optionally substituted C₁-C₆ alkyl, optionally substituted C₃-C₁₀ cycloalkyl, or optionally substituted 4- to 10-membered heterocycloalkyl;
   or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
   each R^{2b} is independently optionally substituted C₁-C₆ alkyl;
   each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or optionally substituted C₁-C₆ alkyl;
   R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
   each R^{5a} is independently hydrogen or optionally substituted C₁-C₆ alkyl;
   each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R^{6′}, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
   each R^{6a} is independently hydrogen or optionally substituted C₁-C₆ alkyl;
   each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4.
Embodiment E3. The compound of Embodiment E1 or Embodiment E2, or a pharmaceutically acceptable salt or solvate thereof, wherein:
   Y¹ is CR⁵;
   Y² is CR⁵;
   Y³ is CR⁵; and
   each R⁵ is independently hydrogen, halogen, —CN, —OR^{5a}, —N(R^{5a})₂, or C₁-C₈ alkyl.
Embodiment E4. The compound of any one of Embodiments E1 to E3, or a pharmaceutically acceptable salt or solvate thereof, wherein:
   X¹ is CR⁶ or N;
   X² is CR⁶; and
   each R⁶ is independently hydrogen, halogen, —CN, —OR^{5a}, —N(R^{5a})₂, or C₁-C₈ alkyl.
Embodiment E5. The compound of any one of Embodiments E1 to E4, or a pharmaceutically acceptable salt or solvate thereof, wherein:
   p is 1.
Embodiment E6. The compound of any one of Embodiments E1 to E5, or a pharmaceutically acceptable salt or solvate thereof, wherein:
   q is 1.
Embodiment E7. The compound of Embodiment E1 or Embodiment E2, having the structure of Formula (II):

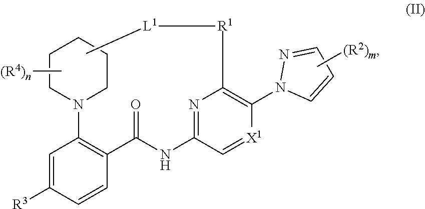

or a pharmaceutically acceptable salt or solvate thereof, wherein:
X¹ is CH or N.
Embodiment E8. The compound of any one of Embodiments E1 to E7, or a pharmaceutically acceptable salt or solvate thereof, wherein:
   R¹ is —NH— or —N(C₁-C₄ alkyl)-.
Embodiment E9. The compound of any one of Embodiments E1 to E8, or a pharmaceutically acceptable salt or solvate thereof, wherein:
   R¹ is —NH—.
Embodiment E10. The compound of any one of Embodiments E1 to E9, or a pharmaceutically acceptable salt or solvate thereof, wherein:
   L¹ is a linker comprising the structure -L^{1a}-L^{1b}-*, where * designates the attachment point to R¹;
   L^{1a} is a bond, —O—, —NH—, —S—, or —SO₂—; and
   L^{1b} is optionally substituted C₄-C₅ alkenylene.
Embodiment E11. The compound of Embodiment E10, or a pharmaceutically acceptable salt or solvate thereof, wherein:
   L^{1a} is a bond or —O—.
Embodiment E12. The compound of Embodiment E10 or E11, or a pharmaceutically acceptable salt or solvate thereof, wherein:
   L^{1a} is a bond.
Embodiment E13. The compound of Embodiment E12, or a pharmaceutically acceptable salt or solvate thereof, wherein:
   L^{1b} is C₅ alkenylene.
Embodiment E14. The compound of Embodiment E10 or E11, or a pharmaceutically acceptable salt or solvate thereof, wherein:
   L^{1a} is —O—.
Embodiment E15. The compound of Embodiment E14, or a pharmaceutically acceptable salt or solvate thereof, wherein:
   L^{1b} is C₄ alkenylene.
Embodiment E16. The compound of Embodiment E10, or a pharmaceutically acceptable salt or solvate thereof, wherein:
   L^{1a} is a bond; and L^{1b} is C₅ alkenylene;
   or L^{1a} is —O—; and L^{1b} is C₄ alkenylene.
Embodiment E17. The compound of Embodiment E10, or a pharmaceutically acceptable salt or solvate thereof, wherein L¹ is —CH₂CH═CHCH₂CH₂—*, —CH₂CH₂CH═CHCH₂—*, or —OCH₂CH═CHCH₂—*, where * designates the attachment point to R¹.
Embodiment E18. The compound of Embodiment E1 or Embodiment E2, having the structure of Formula (IIIa), (IIIb), (IVa), or (IVb):

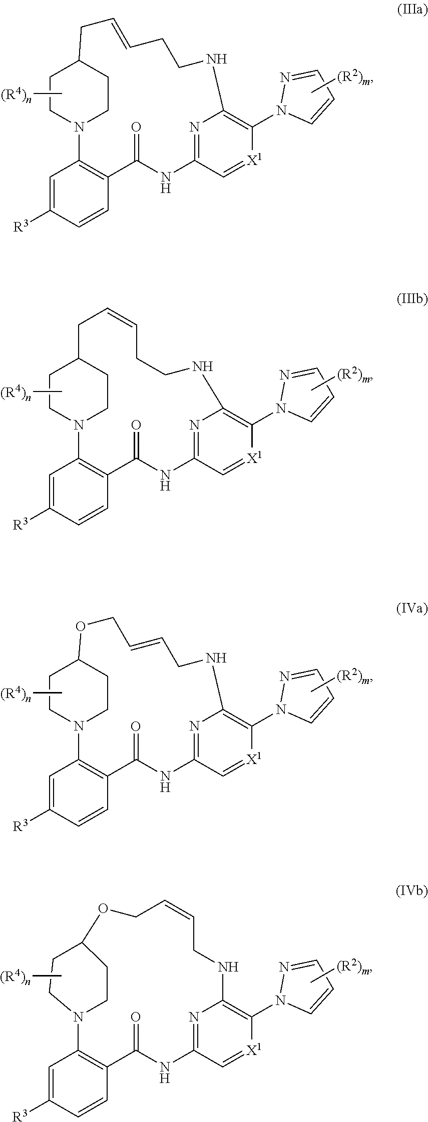

or a pharmaceutically acceptable salt or solvate thereof, wherein:
X is CH or N.
Embodiment E19. The compound of any one of Embodiments E1 to E9, or a pharmaceutically acceptable salt or solvate thereof, wherein:
   L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.
Embodiment E20. The compound of Embodiment E19, or a pharmaceutically acceptable salt or solvate thereof, wherein:
   L¹ is optionally substituted C₄-C₅ heteroalkylene; wherein the terminal atoms of the heteroalkylene are C, and the heteroalkylene comprises 1 heteroatom selected from N, O, and S.
Embodiment E21. The compound of Embodiment E19 or E20, or a pharmaceutically acceptable salt or solvate thereof, wherein:
   L¹ is optionally substituted C₅ heteroalkylene; wherein the terminal atoms of the heteroalkylene are C, and the heteroalkylene comprises 1 O atom.
Embodiment E22. The compound of Embodiment E19, or a pharmaceutically acceptable salt or solvate thereof, wherein L¹ is —CH₂OCH₂CH₂CH₂—* or —CH₂CH₂OCH₂CH₂—*, where * designates the attachment point to R¹.
Embodiment E23. The compound of Embodiment E1 or Embodiment E2, having the structure of Formula (VIa), (VIb), (VIIa), or (VIIb):

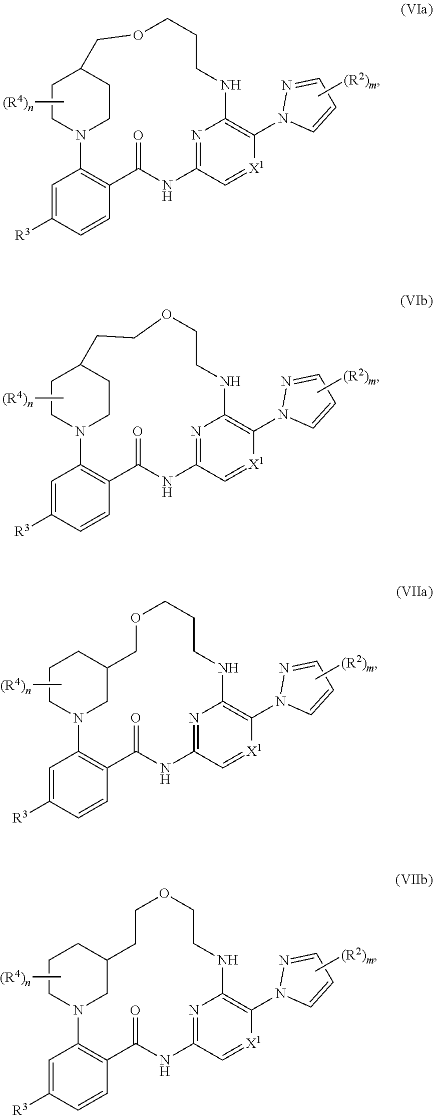

or a pharmaceutically acceptable salt or solvate thereof, wherein:
X¹ is CH or N.
Embodiment E24. The compound of any one of Embodiments E1 to E23, or a pharmaceutically acceptable salt or solvate thereof, wherein:
   X¹ is CH.
Embodiment E25. The compound of any one of Embodiments E1 to E23, or a pharmaceutically acceptable salt or solvate thereof, wherein:
   X¹ is N.
Embodiment E26. The compound of any one of Embodiments E1 to E25, or a pharmaceutically acceptable salt or solvate thereof, wherein:
   each R² is independently halogen, —CN, —OR^{2a}, —N(R^{2a})₂, —C(O)N(R^{2a})₂, —N(R^{2a})C(O)NR^{2a}, —S(O)₂N(R^{2a})₂, —S(O)₂R^{2b}, C₁-C₈ alkyl, or C₁-C₈ hydroxyalkyl;
   each R^{2a} is independently hydrogen or C₁-C₆ alkyl; wherein the alkyl is optionally substituted with 1, 2, or 3 groups independently selected from halogen, —OH, and —CN;
   or two R^{2a} on the same N atom are taken together with the N atom to which they are attached to form a 4- to 6-membered heterocycloalkyl which is optionally substituted with 1, 2, or 3 groups independently selected from halogen, —OH, C₁-C₂ alkyl, and C₁-C₂ haloalkyl;
   and
   each R^{2b} is independently C₁-C₆ alkyl.
Embodiment E27. The compound of any one of Embodiments E1 to E26, or a pharmaceutically acceptable salt or solvate thereof, wherein:
   each R² is independently —F, —Cl, —CN, —OR^{2a}, —C(O)N(R^{2a})₂, —S(O)₂R^{2b}, or C₁-C₄ hydroxyalkyl;
   each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, or —CH(CH₃)CH₂OH;
   or two R^{2a} on the same N atom are taken together with the N atom to which they are attached to form a 4- to 6-membered heterocycloalkyl selected from an azetidinyl, pyrrolidinyl, piperidinyl, and morpholinyl, wherein the heterocycloalkyl is optionally substituted with 1, 2, or 3 groups independently selected from —F, —OH, —CH₃, —CH₂F, —CHF₂, and —CF₃; and
   each R^{2b} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment E28. The compound of any one of Embodiments E1 to E25, or a pharmaceutically acceptable salt or solvate thereof, wherein:
   each R² is independently —Cl, —CN, —OCH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂OH,

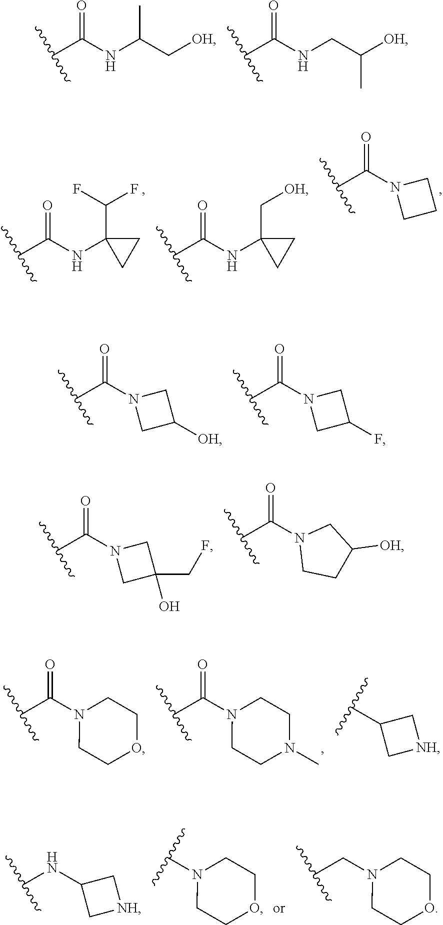

Embodiment E29. The compound of any one of Embodiments E1 to E28, or a pharmaceutically acceptable salt or solvate thereof, wherein:
   m is 1.
Embodiment E30. The compound of any one of Embodiments E1 to E29, or a pharmaceutically acceptable salt or solvate thereof, wherein:
   each R⁴ is independently halogen, —CN, C₁-C₆ alkyl, or C₁-C₆ fluoroalkyl.
Embodiment E31. The compound of any one of Embodiments E1 to E30, or a pharmaceutically acceptable salt or solvate thereof, wherein:
   each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment E32. The compound of any one of Embodiments E1 to E31, or a pharmaceutically acceptable salt or solvate thereof, wherein:
   each R⁴ is independently —CN or —CH₃.
Embodiment E33. The compound of any one of Embodiments E1 to E32, or a pharmaceutically acceptable salt or solvate thereof, wherein: n is 1.
Embodiment E34. The compound of any one of Embodiments E1 to E33, or a pharmaceutically acceptable salt or solvate thereof, wherein:
   R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
   each R^{3a} is independently hydrogen or C₁-C₆ alkyl;
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl;
   or R^{3a} and R^{3b} are taken together with the intervening atoms to which they are attached to form an optionally substituted 4- to 8-membered heterocycloalkyl.
Embodiment E35. The compound of any one of Embodiments E1 to E34, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
   or R^{3a} and R^{3b} are taken together with the intervening atoms to which they are attached to form an optionally substituted 5- to 6-membered heterocycloalkyl.
Embodiment E36. The compound of Embodiment E35, or a pharmaceutically acceptable salt or solvate thereof, wherein:
   R³ is —NR^{3a}(SO₂R^{3b}) or —SO₂NR^{3a}R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment E37. The compound of Embodiment E36, or a pharmaceutically acceptable salt or solvate thereof, wherein:
   R^{3a} is hydrogen or C₁-C₃ alkyl; and
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment E38. The compound of Embodiment E35, or a pharmaceutically acceptable salt or solvate thereof, wherein:
   R^{3a} is hydrogen, —CH₃, or —CH₂CH₃; and
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂CH₂C≡CH, —CH₂CH₂CH₂CH₂C≡CH,

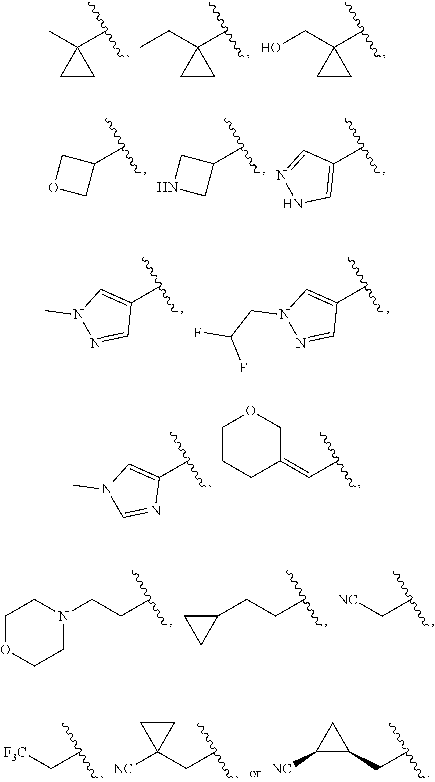

Embodiment E39. The compound of Embodiment E35, or a pharmaceutically acceptable salt or solvate thereof, wherein:
   R^{3a} and R^{3b} are taken together with the intervening atoms to which they are attached to form an optionally substituted 5- to 6-membered heterocycloalkyl.
Embodiment E40. The compound of Embodiment E39, or a pharmaceutically acceptable salt or solvate thereof, wherein:
   R^{3a} and R^{3b} are taken together with the intervening atoms to which they are attached to form

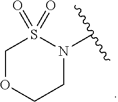 [SMILES: *N1CCOCS1(=O)=O]

Embodiment E41. The compound of any one of Embodiments E1 to E34, or a pharmaceutically acceptable salt or solvate thereof, wherein:
   R³ is —N(R^{3a})₂; and
   each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.
Embodiment E42. The compound of any one of Embodiments E1 to E34, or a pharmaceutically acceptable salt or solvate thereof, wherein:
   R³ is —SO₂R^{3b}; and
   R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic 5-membered heteroaryl.
Embodiment E43. The compound of Embodiment E42, or a pharmaceutically acceptable salt or solvate thereof, wherein:
   R^{3b} is C₁-C₃ alkyl, C₁-C₃ hydroxyalkyl, or C₃-C₆ cycloalkyl which is substituted by a C₁-C₃ alkyl or C₁-C₃ hydroxyalkyl.
Embodiment E44. The compound of Embodiment E42, or a pharmaceutically acceptable salt or solvate thereof, wherein:
   R^{3b} is —CH₃, —CH₂CH₃, —CH₂CH₂CH₃, —CH(CH₃)₂, —CH₂OH, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂,

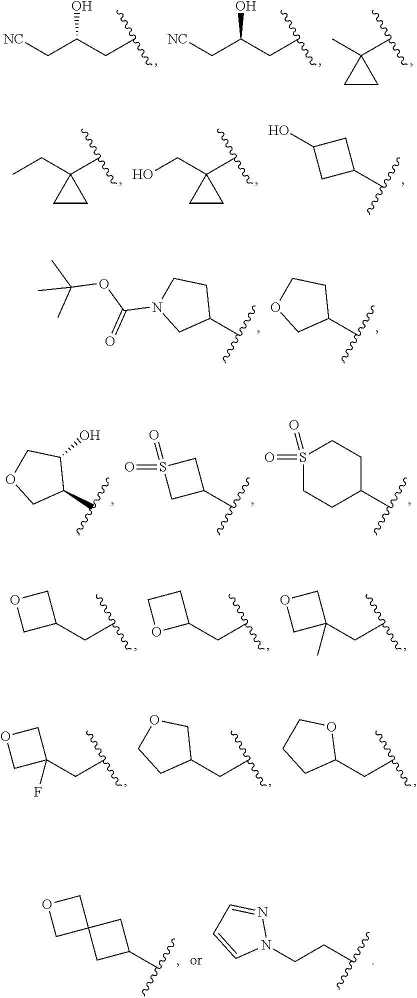

Embodiment E45. The compound of any one of Embodiments E1 to E34, or a pharmaceutically acceptable salt or solvate thereof, wherein:
   R³ is —NR^{3a}C(O)—C(O)R^{3b};
   R^{3a} is hydrogen or C₁-C₆ alkyl; and
   R^{3b} is C₁-C₆ alkyl, C₁-C₆ hydroxyalkyl, or optionally substituted C₃-C₆ cycloalkyl.
Embodiment E46. The compound of Embodiment E45, or a pharmaceutically acceptable salt or solvate thereof, wherein:
   R^{3a} is hydrogen, —CH₃, —CH₂CH₃, —CH₂CH₂CH₃, or —CH(CH₃)₂; and
   R^{3b} is —CH₃, —CH₂CH₃, —CH₂CH₂CH₃, —CH(CH₃)₂, —CH₂OH, —CH₂CH₂OH, —CH₂CH₂CH₂OH,

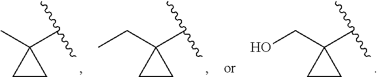

Embodiment E47. The compound of any one of Embodiments E1 to E33, or a pharmaceutically acceptable salt or solvate thereof, wherein:
   R³ is —NH₂, —NHSO₂CH₂CH₃, —N(CH₃)SO₂CH₂CH₃, —NHSO₂CH₂CH₂OH, —N(CH₃)SO₂CH₂CH₂OH, —NHSO₂CH₂CN, —NHSO₂CH₂CF₃, —NHSO₂CH₂CH₂C≡CH, —NHSO₂CH₂CH₂CH₂CH₂C≡CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂CH₂CH₂OCH₃, —SO₂CH₂CH₂CH₂OH, —SO₂CH₂C(CH₃)₂OH, —SO₂CH₂CH₂C(CH₃)₂OH, —SO₂CH(CH₃)CH₂OH, —SO₂CH₂CH₂SO₂CH₃, —SO₂CH₂CH₂OCH₂CN, —SO₂CH₂CH(CH₃)OH, —SO₂CH₂CH₂CN, —SO₂CH(CH₃)CN, —SO₂CH₂CH₂C(═CH₂)CN, —SO₂CH₂C(CH₃)(OH)CH₂OH, —SO₂CH₂C(═O)NH₂, —SO₂NHCH₂CH₂OH,

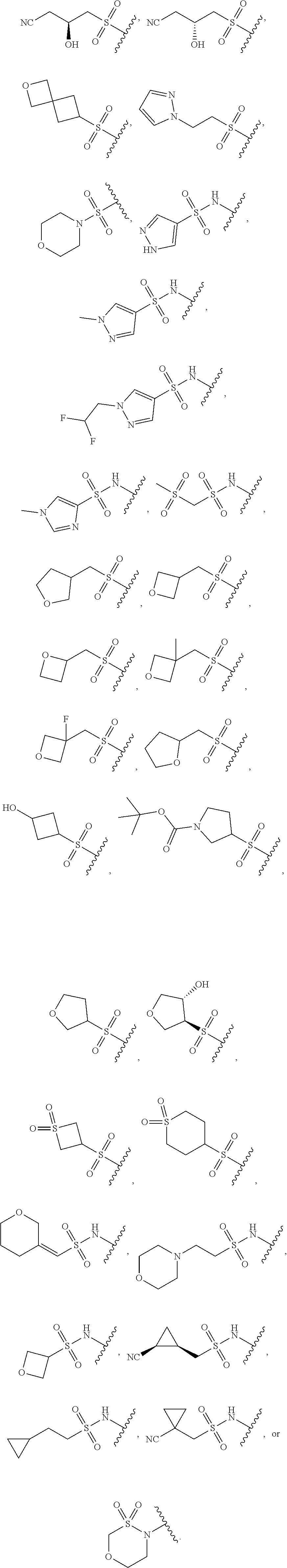

Embodiment E48. The compound of any one of Embodiments E1 to E33, or a pharmaceutically acceptable salt or solvate thereof, wherein:
   R³ is —NHSO₂CH₂CH₃, —NHSO₂CH₂CH₂OH, —SO₂CH₂CH₃, or —SO₂CH₂CH₂OH.
Embodiment E49. The compound of any one of Embodiments E1 to E33, or a pharmaceutically acceptable salt or solvate thereof, wherein:
   R³ is —NHSO₂CH₂CH₃ or —NHSO₂CH₂CH₂OH.

### EXAMPLES

The following illustrative examples are representative of embodiments of the stimulation, systems, and methods described herein and are not meant to be limiting in any way.

### I. Chemical Synthesis

In some instances, a compound provided herein (e.g., a compound provided in Table 1) is synthesized as described herein.

Synthesis of Compounds

### Intermediate A. 3-bromo-N²-(but-3-en-1-yl)pyridine-2,6-diamine

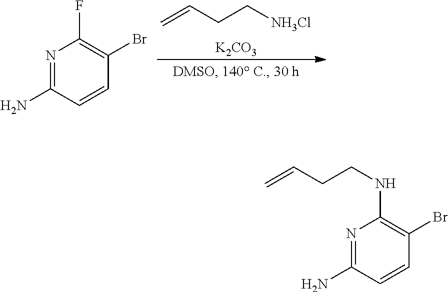

To a flask was added 3-Buten-1-amine, hydrochloride (11.3 g, 105 mmol), 5-Bromo-6-fluoro-pyridin-2-ylamine (10.0 g, 52.4 mmol), and potassium carbonate (18.1 g, 131 mmol). The flask was placed under a N₂ atmosphere and DMSO (200 mL) was added. The reaction mixture was then heated to 140° C. After 30 hour, the reaction mixture was diluted with water and extracted with EtOAc (4×). The combined organic layers were washed with brine (3×) dried over magnesium sulfate, and concentrated. Purification via Isco column chromatography (0-20% EtOAc in heptane) provided 3-bromo-N²-(but-3-en-1-yl)pyridine-2,6-diamine (10.9 g, 86.0% yield). LCMS (ESI-MS) m/z=242.0 [M+H]⁺.

### Intermediate B. N²-(but-3-en-1-yl)-3-(1H-pyrazol-1-yl)pyridine-2,6-diamine

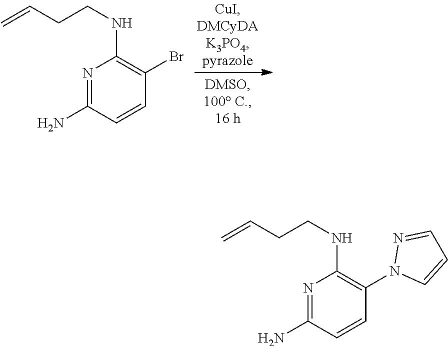 [SMILES: C=CCCNc1nc(N)ccc1Br ; C=CCCNc1nc(N)ccc1-n1cccn1]

CuI (47 g, 0.25 mol) was added to the mixture of 3-bromo-N²-(but-3-en-1-yl)pyridine-2,6-diamine (40 g, 0.17 mol), K₃PO₄ (110 g, 0.51 mol), 1H-pyrazole (34 g, 0.50 mol) and (1R,2R)—N¹,N²-dimethylcyclohexane-1,2-diamine (35 g, 0.25 mol) in DMSO (400 mL) under N₂ atmosphere. The mixture was stirred at 100° C. overnight. The reaction mixture was diluted with water (2 L). The resulting mixture was extracted with EA (3×2 L). The combined organic layers were washed with brine (3 L), dried over anhydrous Na₂SO₄, and concentrated. The residue was purified by silica gel column chromatography, eluted with EA:PE=1:4 to afford N²-(but-3-en-1-yl)-3-(1H-pyrazol-1-yl)pyridine-2,6-diamine (20.1 g, 53% yield). LCMS (ESI-MS) m/z=230.1 [M+H]⁺.

### Intermediate C. 2-(4-allylpiperidin-1-yl)-4-chlorobenzoic acid

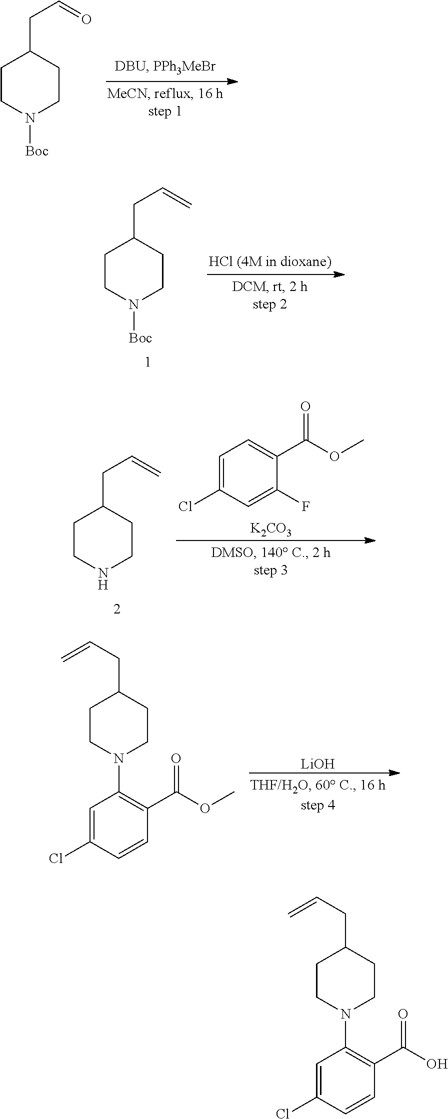

Step 1. tert-butyl 4-allylpiperidine-1-carboxylate: DBU (54 g, 53 mL, 0.35 mol) was added to a mixture of tert-butyl 4-(2-oxoethyl)piperidine-1-carboxylate (40 g, 0.18 mol) and Methyltriphenylphosphonium Bromide (130 g, 0.35 mol) in Acetonitrile (400 mL) The resulting mixture was heated to 85° C. and stirred for 16 hours. After cooling to rt, the reaction mixture was diluted with water (300 mL) and extracted with EA (3×200 mL). The combined organic layers were washed with brine (3×200 mL), dried with anhydrous Na₂SO₄, and concentrated. The residue was purified via silica gel column chromatography (EA:PE=1:10) to afford tert-butyl 4-allylpiperidine-1-carboxylate (34 g, 86% yield). LCMS (ESI-MS) m/z=226.2 [M+H]⁺.

Step 2. 4-allylpiperidine: HCl in 1,4-dioxane (4 M, 36 mL) was added to a stirred mixture of tert-butyl 4-allylpiperidine-1-carboxylate (34 g, 0.15 mol) in DCM (68 mL). The resulting mixture was stirred at room temperature for 2 hours and concentrated to afford 4-allylpiperidine (18.6 g). This material was used for the next step without further purification. LCMS (ESI-MS) m/z=126.2 [M+H]⁺.

Step 3. Methyl 2-(4-allylpiperidin-1-yl)-4-chlorobenzoate: K₂CO₃ (61.3 g, 443 mmol) was added to a stirred mixture of 4-allylpiperidine (18.5 g, 148 mmol) and methyl 4-chloro-2-fluorobenzoate (27.9 g, 148 mmol) in DMSO (190 mL). The resulting mixture was stirred at 140° C. for 2 hours, diluted with water (600 mL), and extracted with EA (3×200 mL). The combined organic layers were washed with brine (300 mL×2), dried with anhydrous Na₂SO₄, and concentrated. The residue was purified with silica gel column, eluted with EA:PE=1:3 to afford the desired product methyl 2-(4-allylpiperidin-1-yl)-4-chlorobenzoate (23.8 g, 54.8% yield). LCMS (ESI-MS) m/z=294.1 [M+H]⁺.

Step 4. 2-(4-allylpiperidin-1-yl)-4-chlorobenzoic acid: LiOH (5.82 g, 243 mmol) in Water (60 mL) was added to a mixture of methyl 2-(4-allylpiperidin-1-yl)-4-chlorobenzoate (23.8 g, 81.0 mmol) in THF (240 mL). The resulting mixture was heated to 60° C. and stirred for 16 hours. After cooling to rt. The mixture was acidified to pH 2 with 1 M HCl. The resulting mixture was extracted with EtOAc (3×200 mL). The combined organic layers were washed with brine (2×200 mL), dried over anhydrous Na₂SO₄, filtered, and concentrated to afford 2-(4-allylpiperidin-1-yl)-4-chlorobenzoic acid (21.5 g, 94.9% yield). This material was used without further purification. LCMS (ESI-MS) m/z=280.2 [M+H]⁺.

### Intermediate D. N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide

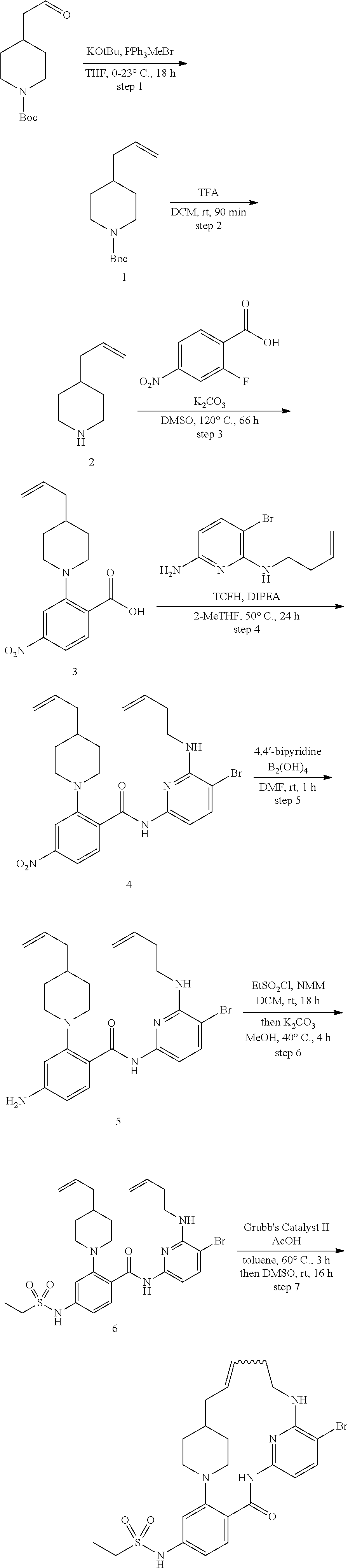

Step 1. tert-butyl 4-allylpiperidine-1-carboxylate: KOtBu (8.89 g, 79.2 mmol) was slowly added to Methyltriphenylphosphonium Bromide (28.3 g, 79.2 mmol) in THF (140 mL) at 0° C., then the reaction mixture was warmed to rt and stirred for 1 hour. A solution of 4-(2-oxoethyl)piperidine-1-carboxylic acid, tert-butyl ester (9.00 g, 39.6 mmol) in THF (20 mL) was then added slowly and the reaction mixture was stirred at rt. After 18 hour, the reaction mixture was diluted with diethyl ether, filtered through celite, and concentrated. Purification via Isco column chromatography (0-20% EtOAc in heptane) provided tert-butyl 4-allylpiperidine-1-carboxylate (8.75 g, 98.1% yield).

Step 2. 4-allylpiperidine: tert-butyl 4-allylpiperidine-1-carboxylate (18.8 g, 83.4 mmol) was stirred with TFA (47.6 g, 32.1 mL, 417 mmol) in DCM (250 mL) at rt for 90 min, then concentrated in vacuo. The crude material was used for the next step without further purification.

Step 3. 2-(4-allylpiperidin-1-yl)-4-nitrobenzoic acid: To the crude material from the previous step was added 2-fluoro-4-nitrobenzoic acid (15.4 g, 83.4 mmol), K₂CO₃ (30.0 g, 217 mmol), and DMSO (300 mL). The reaction mixture was then heated to 120° C. After 66 hour, the reaction mixture was diluted with brine (500 mL) and extracted with DCM (5×). The combined organic layers were washed with brine (3×), dried over MgSO₄, and concentrated. The crude material was purified via Isco column chromatography (0-100% 75:25:1 EtOAc/EtOH/AcOH in heptane) to provide 2-(4-allylpiperidin-1-yl)-4-nitrobenzoic acid (20.8 g, 85.9% yield). LCMS (ESI-MS) m/z=291.1 [M+H]⁺.

Step 4. 2-(4-allylpiperidin-1-yl)-N-(5-bromo-6-(but-3-en-1-ylamino)pyridin-2-yl)-4-nitrobenzamide: To a flask was added 2-(4-allylpiperidin-1-yl)-4-nitrobenzoic acid (5.00 g, 17.2 mmol), 3-bromo-N²-(but-3-en-1-yl)pyridine-2,6-diamine (5.00 g, 20.7 mmol), N-(chloro(dimethylamino)methylene)-N-methylmethanaminium hexafluorophosphate(V) (6.28 g, 22.4 mmol), DIPEA (4.45 g, 6.00 mL, 34.4 mmol), and 2-MeTHF (90.0 mL). The reaction mixture was then stirred at rt. After 24 hour the reaction mixture was quenched with 1 M aq. NaOH, diluted with water, and extracted with EtOAc (3×). The combined organic layers were washed with brine, dried over MgSO₄, and concentrated. Purification via Isco column chromatography (0-20% EtOAc on heptane) provided 2-(4-allylpiperidin-1-yl)-N-(5-bromo-6-(but-3-en-1-ylamino)pyridin-2-yl)-4-nitrobenzamide (5.77 g, 65.1% yield). LCMS (ESI-MS) m/z=514.1 [M+H]⁺.

Step 5. 2-(4-allylpiperidin-1-yl)-4-amino-N-(5-bromo-6-(but-3-en-1-ylamino)pyridin-2-yl)benzamide: To a flask was added 2-(4-allylpiperidin-1-yl)-N-(5-bromo-6-(but-3-en-1-ylamino)pyridin-2-yl)-4-nitrobenzamide (6.99 g, 1 Eq, 13.6 mmol), Hypodiboric acid (4.87 g, 4 Eq, 54.4 mmol) and 4,4′-Dipyridyl (53.1 mg, 0.025 Eq, 340 μmol). The reaction vial was placed under a N₂ atmosphere, then DMF (60 mL) was added. The reaction mixture was then stirred at 23° C. After 1 hour the reaction mixture was diluted with water and extracted with EtOAc (3×). The combined organic layers were washed with sat. aq. NaHCO₃ then brine, dried over MgSO₄, and concentrated. Purification via Isco column chromatography (0-100% EtOAc on heptane) provided 2-(4-allylpiperidin-1-yl)-4-amino-N-(5-bromo-6-(but-3-en-1-ylamino)pyridin-2-yl)benzamide (6.00 g, 91.1% yield). LCMS (ESI-MS) m/z=484.1 [M+H]⁺.

Step 6. 2-(4-allylpiperidin-1-yl)-N-(5-bromo-6-(but-3-en-1-ylamino)pyridin-2-yl)-4-(ethylsulfonamido)benzamide: To a flask was added 2-(4-allylpiperidin-1-yl)-4-amino-N-(5-bromo-6-(but-3-en-1-ylamino)pyridin-2-yl)benzamide (6.0 g, 12 mmol), THF (50 mL), and 4-methylmorpholine (7.5 g, 8.2 mL, 74 mmol). Ethylsulfonyl Chloride (6.4 g, 4.7 mL, 50 mmol) in THF (10 mL) was added slowly and the reaction mixture was stirred at rt. After 18 hour, K₂CO₃ (17 g, 0.12 mol) and MeOH (30 mL) were added and the reaction mixture heated to 40° C. After 4 hour, the reaction mixture was diluted with brine and extracted with DCM (5×). The combined organic layers were washed with brine, dried over MgSO₄, and concentrated. Purification via Isco column chromatography (0-10% MeOH in DCM) provided 2-(4-allylpiperidin-1-yl)-N-(5-bromo-6-(but-3-en-1-ylamino)pyridin-2-yl)-4-(ethylsulfonamido)benzamide (2.1 g, 29% yield). LCMS (ESI-MS) m/z=577.6 [M+H]⁺.

Step 7. N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide: To a flask was added 2-(4-allylpiperidin-1-yl)-N-(5-bromo-6-(but-3-en-1-ylamino)pyridin-2-yl)-4-(ethylsulfonamido)benzamide (2.1 g, 3.6 mmol), toluene (300 mL) and Benzylidene(dichloro)(1,3-dimesityl-2-imidazolidinylidene)ruthenium-tricyclohexylphosphine (1:1) (0.62 g, 0.73 mmol) under a N₂ atmosphere. The reaction mixture was then heated to 60° C. After 3 hour the reaction mixture was cooled to rt and DMSO (5.7 g, 5.2 mL, 73 mmol) was added and stirred at rt. After 18 h, the reaction mixture was concentrated in vacuo. Purification via Isco column chromatography (0-100% EtOAc on heptane) provided N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (1.5 g, 75% yield). LCMS (ESI-MS) m/z=548.1 [M+H]⁺.

### Intermediate EL. (E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one and Intermediate E2. (Z)-2⁵-amino-5⁵-(1H-pyrazol-1-yl)-4,6-diaza-5(2,6)-pyridina-1(1,4)-piperidina-2(1,2)-benzenacycloundecaphan-9-en-3-one

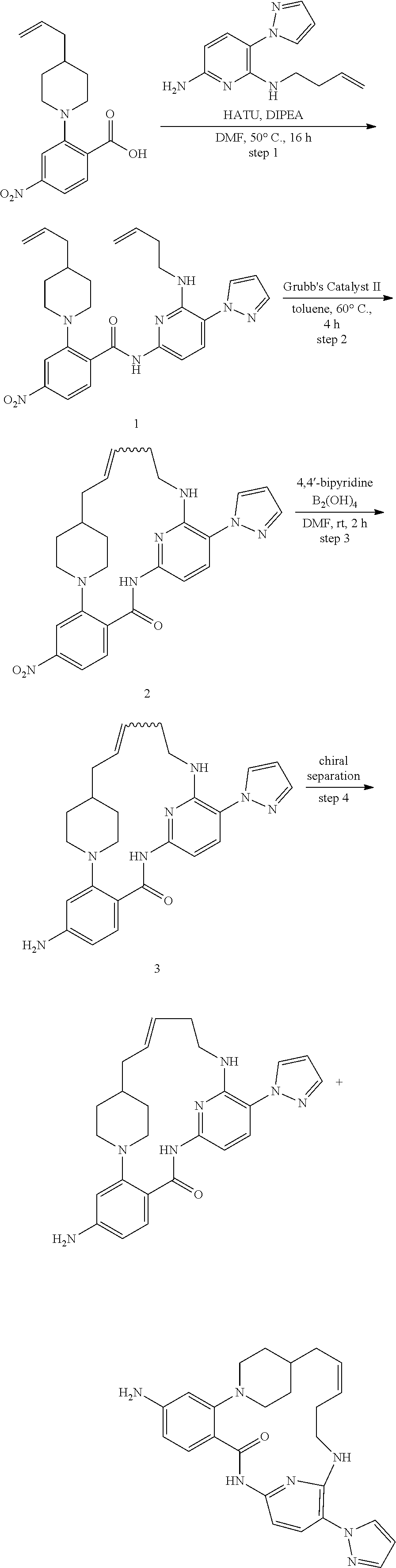

Step 1. 2-(4-allylpiperidin-1-yl)-N-(6-(but-3-en-1-ylamino)-5-(1H-pyrazol-1-yl)pyridin-2-yl)-4-nitrobenzamide: Into a solution of 2-(4-allylpiperidin-1-yl)-4-nitrobenzoic acid (7 g, 0.02 mol) in DMF (100 mL) was added N²-(but-3-en-1-yl)-3-(1H-pyrazol-1-yl)pyridine-2,6-diamine (6 g, 0.02 mol), HATU (26 g, 0.07 mol) and DIEA (9 g, 0.07 mol). The resulting mixture was heated to 50° C. and stirred for 16 hour. After cooling to room temperature, the reaction mixture was diluted with water (100 mL) and extracted with EA (2×100 mL). The combined organic layers were washed with brine (500 mL), dried with anhydrous Na₂SO₄, filtered and concentrated under vacuum. The residue was purified with silica gel column, eluted with PE:EA=4:1 to afford 2-(4-allylpiperidin-1-yl)-N-(6-(but-3-en-1-ylamino)-5-(1H-pyrazol-1-yl)pyridin-2-yl)-4-nitrobenzamide (4 g, 30% yield). LCMS (ESI-MS) m/z=502.3 [M+H]⁺.

Step 2. (E)-2⁵-nitro-5⁵-(1H-pyrazol-1-yl)-4,6-diaza-5(2,6)-pyridina-1(1,4)-piperidina-2(1,2)-benzenacycloundecaphan-9-en-3-one: Into a solution of 2-(4-allylpiperidin-1-yl)-N-(6-(but-3-en-1-ylamino)-5-(1H-pyrazol-1-yl)pyridin-2-yl)-4-nitrobenzamide (6 g, 0.01 mol) in toluene (100 mL) was added Grubb's catalyst 2nd Generation (2 g, 2 mmol) under a nitrogen atmosphere. The resulting mixture was heated to 60° C. and stirred for 4 hours. After cooling to room temperature, the reaction mixture concentrated to afford the crude product. The residue was purified with silica gel column, eluted with PE:EA=8:1 to afford 2⁵-nitro-5⁵-(1H-pyrazol-1-yl)-4,6-diaza-5(2,6)-pyridina-1(1,4)-piperidina-2(1,2)-benzenacycloundecaphan-9-en-3-one (2.5 g, 40% yield). LCMS (ESI-MS) m/z=474.2 [M+H]⁺.

Step 3. 2⁵-amino-5⁵-(1H-pyrazol-1-yl)-4,6-diaza-5(2,6)-pyridina-1(1,4)-piperidina-2(1,2)-benzenacycloundecaphan-9-en-3-one: Into a solution of 2⁵-nitro-5⁵-(1H-pyrazol-1-yl)-4,6-diaza-5(2,6)-pyridina-1(1,4)-piperidina-2(1,2)-benzenacycloundecaphan-9-en-3-one (3.00 g, 6.3 mmol) in DMF (20 mL) was added 4,4′-bipyridine (98.90 mg, 0.63 mmol), hypodiboric acid (2.20 g, 25.30 mmol) under nitrogen atmosphere. The resulting solution was stirred for 2 hours at room temperature. Then the reaction mixture was diluted with water (20 mL) and extracted with EA (2×20 mL). The combined organic layers were washed with brine (50 mL), dried over anhydrous Na₂SO₄, filtered and concentrated under vacuum to afford 2⁵-amino-5⁵-(1H-pyrazol-1-yl)-4,6-diaza-5(2,6)-pyridina-1(1,4)-piperidina-2(1,2)-benzenacycloundecaphan-9-en-3-one (2.3 g 82% yield). LCMS (ESI-MS) m/z=444.2 [M+H]⁺.

Step 4. (E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one and (Z)-2⁵-amino-5⁵-(1H-pyrazol-1-yl)-4,6-diaza-5(2,6)-pyridina-1(1,4)-piperidina-2(1,2)-benzenacycloundecaphan-9-en-3-one: The material from the previous step (2.3 g, 5 mmol) was dissolved with MeOH and purified by Chiral-Prep-HPLC with the following conditions: Column: (R, R); Mobile Phase A: Hex(0.1% DEA):(MeOH:DCM=1:1)=20:80; Flow rate: 1 mL/min; Gradient: isocratic; Injection Volume: 2 μl mL to afford:

First eluting isomer: (E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one (1.3 g, 56% yield). LCMS (ESI-MS) m/z=444.2 [M+H]⁺.

Second eluting isomer: (Z)-2⁵-amino-5⁵-(1H-pyrazol-1-yl)-4,6-diaza-5(2,6)-pyridina-1(1,4)-piperidina-2(1,2)-benzenacycloundecaphan-9-en-3-one (200 mg, 8% yield). LCMS (ESI-MS) m/z=444.2 [M+H]⁺.

### Intermediate F. 2⁵-chloro-5⁵-(1H-pyrazol-1-yl)-4,6-diaza-5(2,6)-pyridina-1(1,4)-piperidina-2(1,2)-benzenacycloundecaphan-9-en-3-one

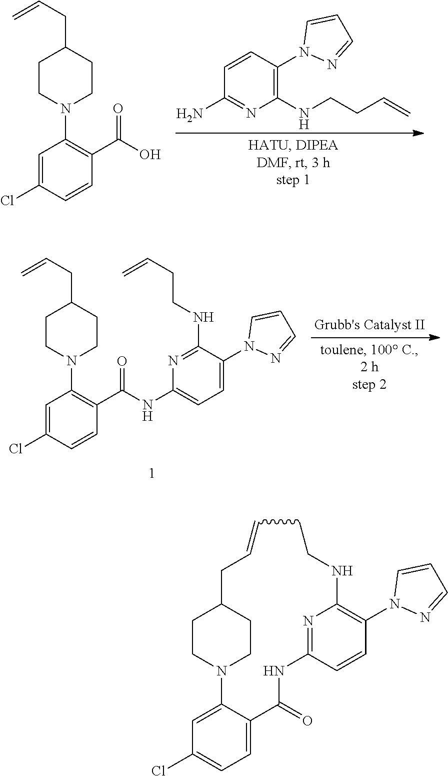 [SMILES: C=CCC1CCN(c2cc(Cl)ccc2C(=O)O)CC1 ; C=CCCNc1nc(N)ccc1-n1cccn1 ; C=CCCNc1nc(NC(=O)c2ccc(Cl)cc2N2CCC(CC=C)CC2)ccc1-n1cccn1 ; O=C1Nc2ccc(-n3cccn3)c(n2)NCCC=CCC2CCN(CC2)c2cc(Cl)ccc21]

Step 1. 2-(4-allylpiperidin-1-yl)-N-(6-(but-3-en-1-ylamino)-5-(1H-pyrazol-1-yl)pyridin-2-yl)-4-chlorobenzamide: DIPEA (1.7 g, 2.3 mL, 13 mmol) was added to a stirred mixture of 2-(4-allylpiperidin-1-yl)-4-chlorobenzoic acid (1.2 g, 4.4 mmol), N²-(but-3-en-1-yl)-3-(1H-pyrazol-1-yl)pyridine-2,6-diamine (1.1 g, 4.8 mmol) and HATU (3.3 g, 8.7 mmol) in DMF (20 mL). The resulting solution was stirred at room temperature for 3 hours, quenched by addition of water (60 mL) and extracted with EA (3×20 mL). The combined organic layers were washed with brine (3×30 mL), dried with anhydrous Na₂SO₄, filtered and concentrated under vacuum. The residue was purified with silica gel chromatography, eluted with EA:PE=1:1 to provide 2-(4-allylpiperidin-1-yl)-N-(6-(but-3-en-1-ylamino)-5-(1H-pyrazol-1-yl)pyridin-2-yl)-4-chlorobenzamide (1.2 g, 56% yield). LCMS (ESI-MS) m/z=491.3 [M+H]⁺.

Step 2. 2⁵-chloro-5⁵-(1H-pyrazol-1-yl)-4,6-diaza-5(2,6)-pyridina-1(1,4)-piperidina-2(1,2)-benzenacycloundecaphan-9-en-3-one: Benzylidene(dichloro)(1,3-dimesityl-2-imidazolidinylidene)ruthenium-tricyclohexylphosphine (1:1) (0.38 g, 0.45 mmol) was added to a stirred mixture of 2-(4-allylpiperidin-1-yl)-N-(6-(but-3-en-1-ylamino)-5-(1H-pyrazol-1-yl)pyridin-2-yl)-4-chlorobenzamide (1.1 g, 2.2 mmol) in toluene (40 mL). The resulting solution was stirred at 100° C. for 2 hours, quenched by addition of water (20 mL) and extracted with EA (3×20 mL). The combined organic layers were washed with brine (300 mL), dried with anhydrous Na₂SO₄, filtered and concentrated under vacuum. The residue was purified with silica gel column, eluted with EA:PE=1:1 to provide 2⁵-chloro-5⁵-(1H-pyrazol-1-yl)-4,6-diaza-5(2,6)-pyridina-1(1,4)-piperidina-2(1,2)-benzenacycloundecaphan-9-en-3-one (550 mg, 53% yield). LCMS (ESI-MS) m/z=463.2 [M+H]⁺.

### Intermediate G. 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid

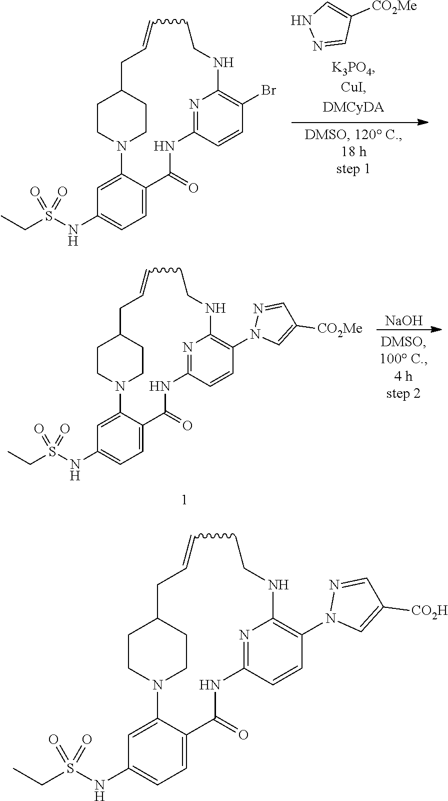 [SMILES: CCS(=O)(=O)Nc1ccc2c(c1)N1CCC(CC=CCCNc3nc(ccc3Br)NC2=O)CC1 ; COC(=O)c1cn[nH]c1 ; CCS(=O)(=O)Nc1ccc2c(c1)N1CCC(CC=CCCNc3nc(ccc3-n3cc(C(=O)OC)cn3)NC2=O)CC1 ; "NaOH" ; CCS(=O)(=O)Nc1ccc2c(c1)N1CCC(CC=CCCNc3nc(ccc3-n3cc(C(=O)O)cn3)NC2=O)CC1]

Step 1. Methyl 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylate: To a vial was added N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (520 mg, 948 μmol), CuI (181 mg, 948 μmol), Trans-(1r,2r)-n,n\′-bismethyl-1,2-cyclohexanediamine (135 mg, 150 μL, 948 μmol), Methyl 1H-Pyrazole-4-Carboxylate (598 mg, 5 Eq, 4.74 mmol), K₃PO₄ (604 mg, 235 μL, 2.84 mmol), and DMSO (5.0 mL) under a N₂ atmosphere. The reaction mixture was then heated to 120° C. After 18 hour, the reaction mixture was diluted with brine and extracted with DCM (3×). The combined organic layers were washed with brine, dried over MgSO₄, and concentrated. The crude material was used for the next step without further purification. LCMS (ESI-MS) m/z=594.2 [M+H]⁺.

Step 2. 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid: The crude residue from the previous step was dissolved in DMSO (5.0 mL) and aq. NaOH (75.8 mg, 1.90 mL, 1.0 molar, 1.90 mmol) was added. The reaction mixture was heated to 100° C. After 4 hour, the crude reaction was purified directly via preparative HPLC (10-100% MeCN in H₂O with 0.1% TFA) followed by lyophilization to provide 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid (247 mg, 44.9% yield). LCMS (ESI-MS) m/z=580.2 [M+H]⁺.

### Intermediate H. 2-(4-allyl-4-methylpiperidin-1-yl)-4-chlorobenzoic acid

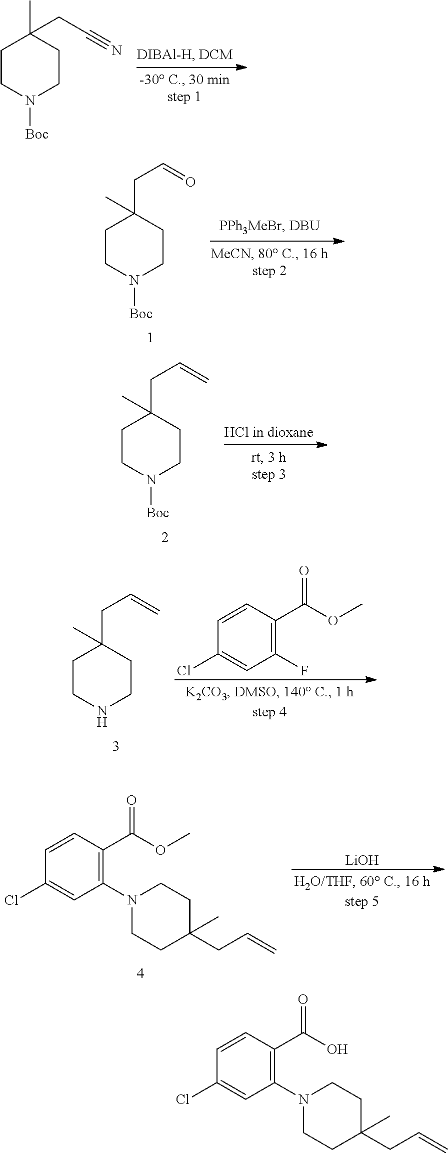

Step 1. tert-butyl 4-methyl-4-(2-oxoethyl)piperidine-1-carboxylate: DIBAL-H (1 M in hexane, 20.98 mL, 21 mmol) was added to a solution of tert-butyl 4-(cyanomethyl)-4-methylpiperidine-1-carboxylate (2 g, 8.39 mmol) in DCM (50 mL) dropwise at −30° C. The resulting solution was stirred at −30° C. for 30 min. The reaction was quenched with MeOH and filtered. The filtrated was diluted with DCM (50 mL), washed with brine (50 mL×2), dried with anhydrous Na₂SO₄ and concentrated to give tert-butyl 4-methyl-4-(2-oxoethyl)piperidine-1-carboxylate (1 g, 50% yield). LCMS (ESI-MS) m/z=242.0 [M+H]⁺.

Step 2. tert-butyl 4-allyl-4-methylpiperidine-1-carboxylate: DBU (1.26 g, 8.29 mmol) was added to a solution of tert-butyl 4-methyl-4-(2-oxoethyl)piperidine-1-carboxylate (1 g, 4.14 mmol) and methyltriphenylphosphanium bromide (2.96 g, 8.29 mmol) in ACN (20 mL). The resulting solution was heated to reflux and stirred for 16 hours. The reaction mixture was diluted with water (30 mL), extracted with EA (30 mL×2). The combined organic layers were combined, washed with brine (30 mL×2), dried with anhydrous Na₂SO₄, filtered and evaporated under vacuum. The residue was purified by silica gel column chromatography, eluted with EA/PE 3:1 to afford tert-butyl 4-allyl-4-methylpiperidine-1-carboxylate (500 mg, 50% yield). LCMS (ESI-MS) m/z=140.4 [M-Boc]⁺.

Step 3. 4-allyl-4-methylpiperidine: Into a 25-mL round-bottom flask, was placed tert-butyl 4-allyl-4-methylpiperidine-1-carboxylate (500 mg, 2.09 mmol) and HCl in 1,4-dioxane (4 M, 5 mL, 20 mmol). The resulting solution was stirred at room temperature for 3 hours. The mixture was evaporated and dried under vacuum to give 4-allyl-4-methylpiperidine (280 mg, 76% yield). LCMS (ESI-MS) m/z=140.1 [M+H]⁺.

Step 4. methyl 2-(4-allyl-4-methylpiperidin-1-yl)-4-chlorobenzoate: K₂CO₃ (14.5 g, 0.108 mol) was added to stirred mixture of 4-allyl-4-methylpiperidine (5 g, 0.036 mol) and methyl 4-chloro-2-fluorobenzoate (6.8 g, 0.036 mol) in DMSO (100 mL). The resulting mixture was heated to 140° C. and stirred for 1 hour. After cooling to room temperature, the reaction mixture was diluted with water (100 mL) and extracted with EA (2×100 mL). The combined organic layers were washed with brine (100 mL), dried over anhydrous Na₂SO₄, filtered and concentrated. The crude residue was purified with silica gel column, eluted with PE:DCM=5:1 to afford methyl 2-(4-allyl-4-methylpiperidin-1-yl)-4-chlorobenzoate (7.0 g, 64% yield). LCMS (ESI-MS) m/z=308.1 [M+H]⁺.

Step 5. 2-(4-allyl-4-methylpiperidin-1-yl)-4-chlorobenzoic acid: LiOH (3.2 g, 0.136 mol) was added to a stirred mixture of methyl 2-(4-allyl-4-methylpiperidin-1-yl)-4-chlorobenzoate (7.0 g, 0.023 mol) in H₂O (20 mL) and THF (60 mL). The resulting mixture was stirred for 16 hours at 60° C. The mixture was allowed to cool to room temperature. The mixture was acidified to pH 6 with conc. HCl. The reaction mixture was diluted with water (60 mL) and extracted with EA (2×60 mL). The combined organic layers were washed with brine (60 mL), dried over anhydrous Na₂SO₄, filtered and concentrated under vacuum to afford 2-(4-allyl-4-methylpiperidin-1-yl)-4-chlorobenzoic acid (6.5 g, 97% yield) which was used without further purification. LCMS (ESI-MS) m/z=294.1 [M+H]⁺.

### Intermediate I. 2⁵-chloro-1⁴-methyl-5⁵-(1H-pyrazol-1-yl)-4,6-diaza-5(2,6)-pyridina-1(1,4)-piperidina-2(1,2)-benzenacycloundecaphan-9-en-3-one

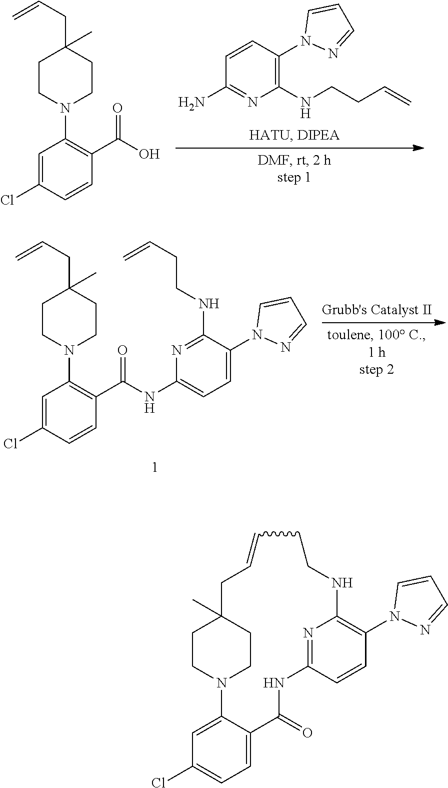 [SMILES: C=CCC1(C)CCN(c2cc(Cl)ccc2C(=O)O)CC1 ; C=CCCNc1nc(N)ccc1-n1cccn1 ; C=CCCNc1nc(NC(=O)c2ccc(Cl)cc2N2CCC(C)(CC=C)CC2)ccc1-n1cccn1 ; CC12CC=CCCNc3nc(ccc3-n3cccn3)NC(=O)c3ccc(Cl)cc3N(CC1)CC2]

Step 1. 2-(4-allyl-4-methylpiperidin-1-yl)-N-(6-(but-3-en-1-ylamino)-5-(1H-pyrazol-1-yl)pyridin-2-yl)-4-chlorobenzamide: 2-(4-allyl-4-methylpiperidin-1-yl)-4-chlorobenzoic acid (6.5 g, 0.022 mol) was added to a stirred mixture of HATU (12.6 g, 0.033 mol), N²-(but-3-en-1-yl)-3-(1H-pyrazol-1-yl)pyridine-2,6-diamine (5.1 g, 0.022 mol) and DIPEA (8.5 g, 0.066 mol) in DMF (50 mL). The resulting mixture was stirred at room temperature. After 2 hour, the reaction mixture was diluted with H₂O (50 mL) and extracted with EA (2×2 mL). The combined organic layers were washed with brine (50 mL), dried over anhydrous Na₂SO₄, filtered and concentrated under vacuum. The residue was purified with silica gel column, eluted with PE:DCM=2:1 to afford methyl 2-(4-allyl-4-methylpiperidin-1-yl)-4-chlorobenzoate (10 g, 89.6% yield). LCMS (ESI-MS) m/z=505.2 [M+H]⁺.

Step 2. 2⁵-chloro-14-methyl-5⁵-(1H-pyrazol-1-yl)-4,6-diaza-5(2,6)-pyridina-1(1,4)-piperidina-2(1,2)-benzenacycloundecaphan-9-en-3-one: 2-(4-allyl-4-methylpiperidin-1-yl)-N-(6-(but-3-en-1-ylamino)-5-(1H-pyrazol-1-yl)pyridin-2-yl)-4-chlorobenzamide (10 g, 0.020 mol) was added to a stirred mixture of Dichloro(1,3-dimesityl-2-imidazolidinylidene)(2-isopropoxybenzylidene)ruthenium (1.25 g, 0.002 mol) in toluene (400 mL). The resulting mixture was stirred for 1 hours at 100° C. The resulting mixture was concentrated under reduced pressure. The residue was purified by silica gel column chromatography, eluted with PE:EA=6:1 to afford 2⁵-chloro-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (6 g, 63.6% yield). LCMS (ESI-MS) m/z=477.2 [M+H]⁺.

### Intermediate J. 2-(4-(allyloxy)-4-methylpiperidin-1-yl)-4-chlorobenzoic acid

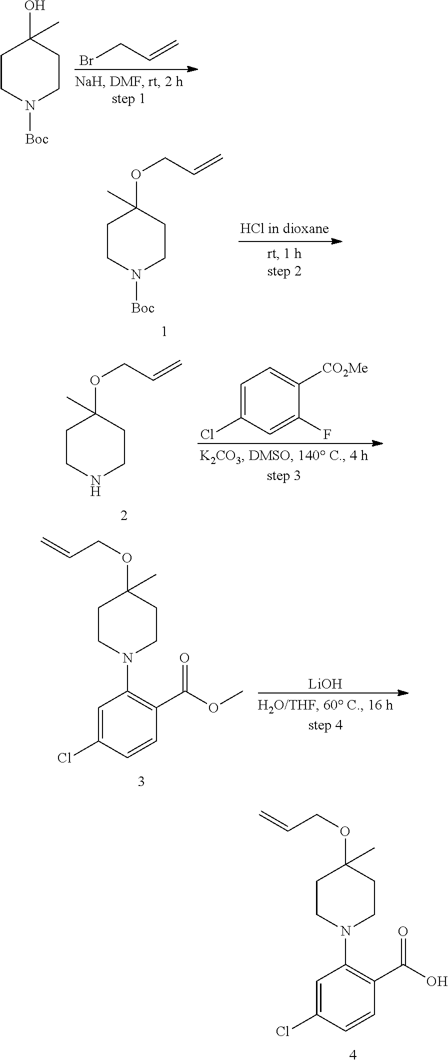

Step 1. tert-butyl 4-(allyloxy)-4-methylpiperidine-1-carboxylate: To a stirred solution of tert-butyl 4-hydroxy-4-methylpiperidine-1-carboxylate (3 g, 13.93 mmol) in anhydrous DMF (30 mL) was added NaH (1 g, 41.80 mmol) at 0° C. and stirred for 1 hour. Then add dropwise 3-bromoprop-1-ene (2.53 g, 20.92 mmol) dissolved in DMF. The resulting mixture was stirred. After 2 hours, the reaction was quenched with water (10 mL) and the aqueous layer was extracted with EA (2×10 mL). The combined organic layers were washed with brine (20 mL), dried over anhydrous Na₂SO₄, filtered and concentrated under vacuum to afford the crude product. The residue was purified by silica gel column chromatography, eluted with PE:EA=4:1 to afford tert-butyl 4-(allyloxy)-4-methylpiperidine-1-carboxylate (2.6 g, 73% yield). LCMS (ESI-MS) m/z=256.2 [M+H]⁺.

Step 2. 4-(allyloxy)-4-methylpiperidine: 4M HCl in 1,4-dioxane (10 mL) was added to a stirred mixture of tert-butyl 4-(allyloxy)-4-methylpiperidine-1-carboxylate (2.6 g, 10.18 mmol) in 1,4-dioxane (10 mL). The resulting mixture was stirred at room temperature for 1 hour and concentrated to afford the crude product 4-(allyloxy)-4-methylpiperidine (1.5 g), which was used for the next step without further purification. LCMS (ESI-MS) m/z=156.1 [M+H]⁺.

Step 3. Methyl 2-(4-(allyloxy)-4-methylpiperidin-1-yl)-4-chlorobenzoate: K₂CO₃ (3.7 g, 27.05 mmol) was added to a mixture of 4-(allyloxy)-4-methylpiperidine (1.4 g, 9.02 mmol) and methyl 4-chloro-2-fluorobenzoate (1.7 g, 9.02 mmol) in DMSO (10 mL). The resulting mixture was heated to 140° C. and stirred for 4 hours. After cooling to room temperature, the reaction mixture was diluted with water (20 mL) and extracted with EA (2×20 mL). The combined organic layers were washed with brine (100 mL), dried over anhydrous Na₂SO₄, filtered and concentrated under vacuum to afford the crude product. The residue was purified by silica gel column chromatography, eluted with PE:EA=4:1 to afford methyl 2-(4-(allyloxy)-4-methylpiperidin-1-yl)-4-chlorobenzoate (2 g, 68% yield). LCMS (ESI-MS) m/z=324.1 [M+H]⁺.

Step 4. 2-(4-(allyloxy)-4-methylpiperidin-1-yl)-4-chlorobenzoic acid: LiOH (1.4 g, 58.6 mmol) was added to a mixture of methyl 2-(4-(allyloxy)-4-methylpiperidin-1-yl)-4-chlorobenzoate (1.9 g, 5.87 mmol) in THF (15 mL) and H₂O (5 mL). The resulting mixture was heated to 50° C. and stirred for 16 hours. After cooling to room temperature, the reaction mixture was diluted with water (20 mL) and extracted with EA (2×20 mL). The combined organic layers were washed with brine (20 mL), dried over anhydrous Na₂SO₄, filtered and concentrated under vacuum to afford 2-(4-(allyloxy)-4-methylpiperidin-1-yl)-4-chlorobenzoic acid (1.8 g, 99% yield). LCMS (ESI-MS) m/z=310.1 [M+H]⁺.

### Intermediate K. 2⁵-chloro-1⁴-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one

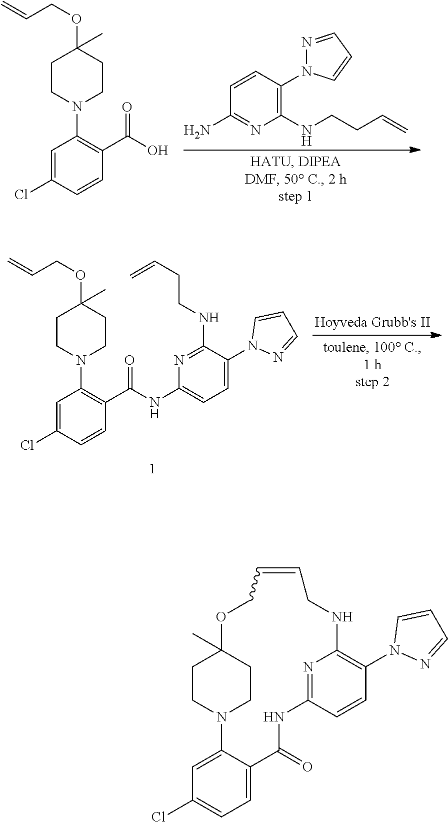 [SMILES: C=CCOC1(C)CCN(c2cc(Cl)ccc2C(=O)O)CC1 ; C=CCCNc1nc(N)ccc1-n1cccn1 ; C=CCCNc1nc(NC(=O)c2ccc(Cl)cc2N2CCC(C)(OCC=C)CC2)ccc1-n1cccn1 ; CC12CCN(CC1)c1cc(Cl)ccc1C(=O)Nc1ccc(-n3cccn3)c(n1)NCC=CCO2]

Step 1. N-(6-(allylamino)-5-(1H-pyrazol-1-yl)pyridin-2-yl)-2-(4-(allyloxy)piperidin-1-yl)-4-chlorobenzamide: Into a solution of 2-(4-(allyloxy)piperidin-1-yl)-4-chlorobenzoic acid (800 mg, 2.70 mmol) in DMF (10 mL) was added N²-allyl-3-(1H-pyrazol-1-yl)pyridine-2,6-diamine (582 mg, 2.70 mmol), HATU (3 g, 7.75 mmol) and DIPEA (1 g, 7.75 mmol). The resulting mixture was heated to 50° C. and stirred for 2 hours. After cooling to room temperature, the reaction mixture was diluted with water (50 mL) and extracted with EA (2×50 mL). The combined organic layers were washed with brine (200 mL), dried with anhydrous Na₂SO₄, filtered and concentrated. The residue was purified with silica gel column, eluted with PE:EA=4:1 to afford N-(6-(allylamino)-5-(1H-pyrazol-1-yl)pyridin-2-yl)-2-(4-(allyloxy)-4-methylpiperidin-1-yl)-4-chlorobenzamide (300 mg, 23% yield). LCMS (ESI-MS) m/z=507.2 [M+H]⁺.

Step 2. 2⁵-chloro-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one: N-(6-(allylamino)-5-(1H-pyrazol-1-yl)pyridin-2-yl)-2-(4-(allyloxy)-4-methylpiperidin-1-yl)-4-chlorobenzamide (280 mg, 0.552 mmol) was added to a stirred mixture of Dichloro(1,3-dimesityl-2-imidazolidinylidene)(2-isopropoxybenzylidene)ruthenium (34.6 mg, 0.055 mmol) in toluene (12 mL). The resulting mixture was stirred for 1 hour at 100° C. The resulting mixture was concentrated under reduced pressure then purified by silica gel column chromatography, eluted with PE:EA=6:1 to afford 2⁵-chloro-1⁴-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (100 mg, 38% yield). LCMS (ESI-MS) m/z=479.2 [M+H]⁺.

### Intermediate L. 2⁵-chloro-5⁵-iodo-4,6-diaza-5(2,6)-pyridina-1(1,4)-piperidina-2(1,2)-benzenacycloundecaphan-9-en-3-one

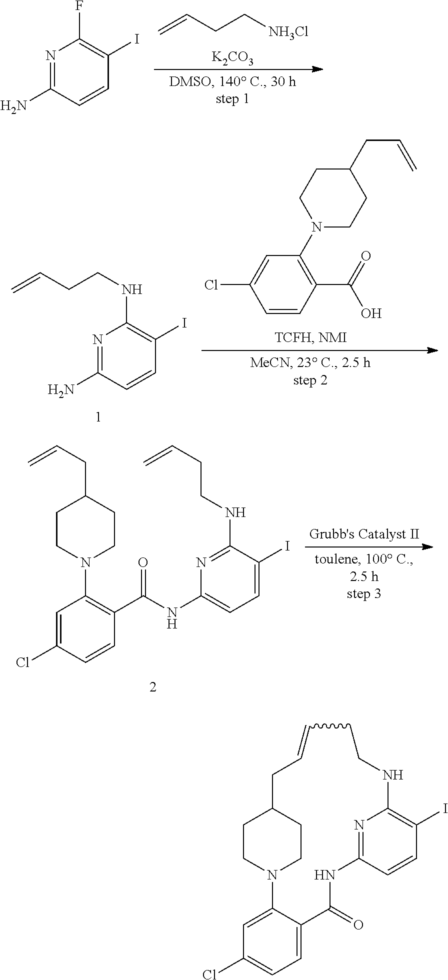

Step 1. N2-(but-3-en-1-yl)-3-iodopyridine-2,6-diamine: To a vial was added 3-buten-1-amine hydrochloride (90.4 mg, 840.3 μmol), 6-fluoro-5-iodopyridin-2-amine (100 mg, 420 μmol), DMSO (0.50 mL), and K₂CO₃ (232 mg, 1.68 mmol). The reaction mixture was then heated to 140° C. After 18 hour, the reaction mixture was diluted with water and extracted with EtOAc (3×). The combined organic layers were washed with brine, dried over MgSO4, and concentrated. Purification via Isco column chromatography (0-100% EtOAc in heptane) provided N²-(but-3-en-1-yl)-3-iodopyridine-2,6-diamine (30 mg, 25% yield). LCMS (ESI-MS) m/z=290.0 [M+H]⁺.

Step 2. 2-(4-allylpiperidin-1-yl)-N-(6-(but-3-en-1-ylamino)-5-iodopyridin-2-yl)-4-chlorobenzamide: To a vial was added 2-(4-allylpiperidin-1-yl)-4-chlorobenzoic acid (80 mg, 1 Eq, 0.29 mmol), N²-(but-3-en-1-yl)-3-iodopyridine-2,6-diamine (91 mg, 0.31 mmol), N-(chloro(dimethylamino)methylene)-N-methylmethanaminium hexafluorophosphate(V) (0.10 g, 0.37 mmol), 1-Methylimidazole(N—) (82 mg, 1.0 mmol), and MeCN (1.5 mL). The reaction mixture was then stirred at rt. After 2.5 hour the reaction mixture was quenched with water and diluted with EtOAc. The organic layer was separated and washed with brine, dried over MgSO₄, and concentrated. Purification via Isco column chromatography (0-20% EtOAc on heptane) provided 2-(4-allylpiperidin-1-yl)-N-(6-(but-3-en-1-ylamino)-5-iodopyridin-2-yl)-4-chlorobenzamide (80 mg, 51% yield). LCMS (ESI-MS) m/z=551.0 [M+H]⁺.

Step 3. 2⁵-chloro-5⁵-iodo-4,6-diaza-5(2,6)-pyridina-1(1,4)-piperidina-2(1,2)-benzenacycloundecaphan-9-en-3-one: To a vial was added 2-(4-allylpiperidin-1-yl)-N-(6-(but-3-en-1-ylamino)-5-iodopyridin-2-yl)-4-chlorobenzamide (80 mg, 0.15 mmol) and Benzylidene(dichloro)(1,3-dimesityl-2-imidazolidinylidene)ruthenium-tricyclohexylphosphine (1:1) (25 mg, 29 μmol). The reaction vial was placed under a N₂ atmosphere then toluene (4.0 mL) was added via syringe. The reaction mixture was then heated to 100° C. After 2.5 hour the reaction mixture was quenched with water and diluted with EtOAc. The organic layer was separated and washed with brine, dried over MgSO₄, and concentrated. Purification via Isco column chromatography (0-20% EtOAc on heptane) provided 25-chloro-5⁵-iodo-4,6-diaza-5(2,6)-pyridina-1(1,4)-piperidina-2(1,2)-benzenacycloundecaphan-9-en-3-one (32 mg, 42% yield). LCMS (ESI-MS) m/z=523.0 [M+H]⁺.

### Intermediate M. 5⁵-bromo-2⁵-chloro-4,6-diaza-5(2,6)-pyridina-1(1,4)-piperidina-2(1,2)-benzenacycloundecaphan-9-en-3-one

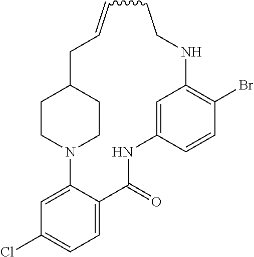 [SMILES: O=C1Nc2ccc(Br)c(c2)NCCC=CCC2CCN(CC2)c2cc(Cl)ccc21]

Intermediate M was synthesized following the procedure for Intermediate L using 5-bromo-6-fluoropyridin-2-amine in step 1.

### Intermediate N. 5⁵-bromo-2⁵-chloro-1⁴-methyl-11-oxa-4,6-diaza-5(2,6)-pyridina-1(1,4)-piperidina-2(1,2)-benzenacycloundecaphan-8-en-3-one

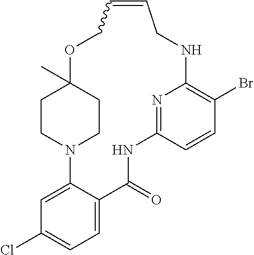 [SMILES: CC12CCN(CC1)c1cc(Cl)ccc1C(=O)Nc1ccc(Br)c(n1)NCC=CCO2]

Intermediate N was synthesized following the procedure for Intermediate K using 3-bromo-N²-(but-3-en-1-yl)pyridine-2,6-diamine in step 1.

### Intermediate O. tert-butyl 3-((3-aminopropoxy)methyl)piperidine-1-carboxylate

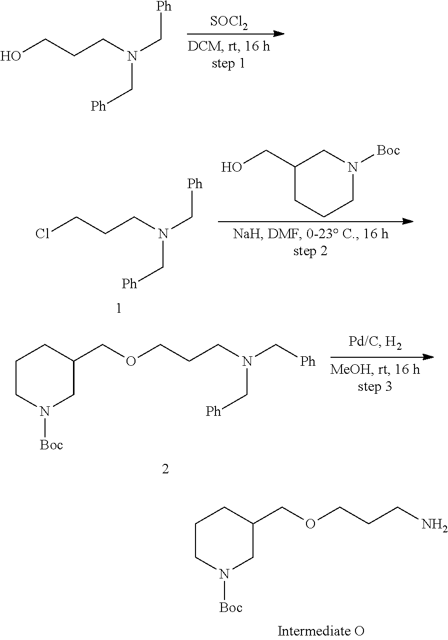

Step 1. N,N-dibenzyl-3-chloropropan-1-amine: 3-(dibenzylamino)propan-1-ol (6.2 g, 24 mmol), a stir bar, SOCl₂ (4.3 g, 36 mmol) and DCM (60 mL) were added to a 100 mL round-bottomed flask, and the resulting mixture stirred at rt for 16 h. The reaction mixture was then concentrated to dryness in vacuo to give a black semi-solid. The semi-solid was taken up in DCM (100 mL), washed with sat NaHCO₃ (100 mL×3) and brine (300 mL), dried over anhydrous MgSO4, filtered, and concentrated to dryness in vacuo to give N,N-dibenzyl-3-chloropropan-1-amine (6.4 g, crude), LCMS (ESI-MS) m/z=274.2 [M+H]⁺.

Step 2. tert-butyl 3-((3-(dibenzylamino)propoxy)methyl)piperidine-1-carboxylate: NaH (60% in mineral oil, 1.9 g, 47 mmol) was added to a stirred mixture of N,N-dibenzyl-3-chloropropan-1-amine (6.4 g, 23 mmol) in DMF (64 mL) at 0° C. After stirring for 30 minutes, tert-butyl 3-(hydroxymethyl)piperidine-1-carboxylate (7.5 g, 35 mmol) was added. The resulting mixture was stirred at room temperature for 16 hours, quenched by addition of saturated aqueous NH₄Cl (200 mL) and extracted with EA (2×200 mL). The combined organic layers were washed with brine (200 mL), dried with anhydrous Na₂SO₄, filtered and concentrated under vacuum. The residue was purified with silica gel column, eluted with EA:PE=10:1 to afford the desired product tert-butyl 3-((3-(dibenzylamino)propoxy)methyl)piperidine-1-carboxylate (7.8 g, 74% yield), LCMS (ESI-MS) m/z=453.4 [M+H]⁺.

Step 3. tert-butyl 3-((3-aminopropoxy)methyl)piperidine-1-carboxylate: Pd/C (4 g, 0.04 mol) was added to a stirred mixture of tert-butyl 3-((3-(dibenzylamino)propoxy)methyl)piperidine-1-carboxylate (7.8 g, 17 mmol) in MeOH (80 mL) under nitrogen atmosphere. The resulting mixture stirred at room temperature for 16 hours under hydrogen atmosphere, filtered and the filtrate was concentrated under vacuum to afford tert-butyl 3-((3-aminopropoxy)methyl)piperidine-1-carboxylate (4.2 g, 89% yield). LCMS (ESI-MS) m/z=273.2 [M+H]⁺.

### Intermediate P. tert-butyl 3-(2-(2-aminoethoxy)ethyl)piperidine-1-carboxylate

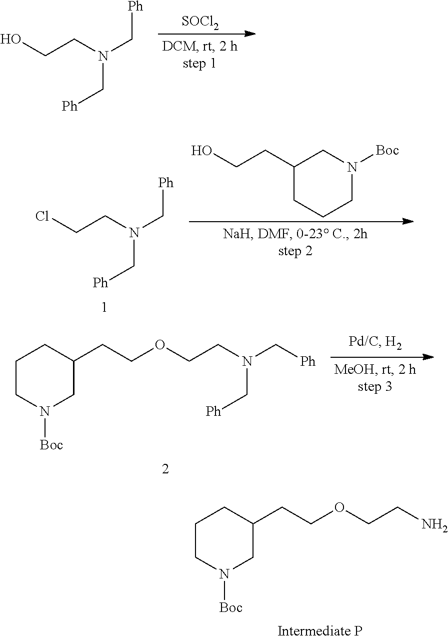

Step 1. N,N-dibenzyl-2-chloroethan-1-amine: To a stirred solution of 2-(dibenzylamino)ethan-1-ol (5 g, 20.7 mmol) in DCM (50 mL) were added SOCl₂(3.7 g, 31.1 mmol) dropwise at 0° C. Then the reaction mixture was stirred for 2 hours at room temperature. After completion of reaction, the reaction mixture was concentrated under reduced pressure to give crude product N,N-dibenzyl-2-chloroethan-1-amine (3 g, 55% yield), LCMS (ESI-MS) m/z=260.1 [M+H]⁺.

Step 2. tert-butyl 3-(2-(2-(dibenzylamino)ethoxy)ethyl)piperidine-1-carboxylate: To a stirred solution of tert-butyl 3-(2-hydroxyethyl)piperidine-1-carboxylate (3 g, 13.1 mmol) in anhydrous DMF (30 mL) was added NaH (942 mg, 39.3 mmol) at 0° C. and stirred for 1 hour. Then N,N-dibenzyl-2-chloroethan-1-amine (3.2 g, 13.1 mmol) dissolved in DMF(10 mL) was added dropwise. The resulting mixture was stirred for 2 hours and monitored by LCMS. After completion of reaction, the reaction was quenched with water (50 mL) and the aqueous layer was extracted with EA (2×50 mL). The residue was purified by silica gel column chromatography, eluted with PE/EA 2:1 to afford tert-butyl 3-(2-(2-(dibenzylamino)ethoxy)ethyl)piperidine-1-carboxylate (1.5 g, 25% yield), LCMS (ESI-MS) m/z=453.3 [M+H]⁺.

Step 3. tert-butyl 3-(2-(2-aminoethoxy)ethyl)piperidine-1-carboxylate: To a solution of tert-butyl 3-(2-(2-(dibenzylamino)ethoxy)ethyl)piperidine-1-carboxylate (1.5 g, 3.3 mmol) in MeOH (20 mL) was added Pd/C (352 mg, 3.3 mmol) under nitrogen atmosphere. The mixture was hydrogenated at room temperature for 2 hours under hydrogen atmosphere using a hydrogen balloon, filtered through a Celite pad and concentrated under reduced pressure to afford tert-butyl 3-(2-(2-aminoethoxy)ethyl)piperidine-1-carboxylate (800 mg, 88% yield), LCMS (ESI-MS) m/z=273.2 [M+H]⁺.

### Intermediate Q. tert-butyl 4-((3-aminopropoxy)methyl)-4-methylpiperidine-1-carboxylate

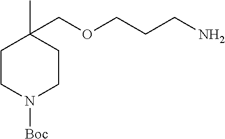 [SMILES: CC1(COCCCN)CCN(C(=O)OC(C)(C)C)CC1]

Intermediate Q was prepared using the same route as Intermediate O with tert-butyl 4-(hydroxymethyl)-4-methylpiperidine-1-carboxylate in step 2. LCMS (ESI-MS) m/z=467.3 [M+H]⁺.

### Intermediate R. tert-butyl 3-((3-aminopropoxy)methyl)-3-methylpiperidine-1-carboxylate

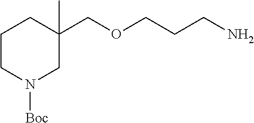 [SMILES: CC1(COCCCN)CCCN(C(=O)OC(C)(C)C)C1]

Intermediate R was prepared using the same route as Intermediate O with tert-butyl 3-(hydroxymethyl)-3-methylpiperidine-1-carboxylate in step 2. LCMS (ESI-MS) m/z=287.1 [M+H]⁺.

### Intermediate S. N-allyl-3-bromopyridine-2,6-diamine

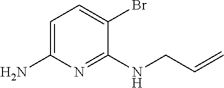

Intermediate S was synthesized following the procedure for Intermediate A using allylamine as the nucleophile.

### Intermediate T. N²-allyl-3-(1H-pyrazol-1-yl)pyridine-2,6-diamine

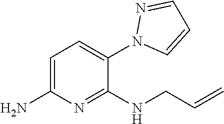 [SMILES: C=CCNc1nc(N)ccc1-n1cccn1]

Intermediate T was synthesized following the procedure for Intermediate B using Intermediate S as the starting substrate.

### Intermediate U. 2-(4-allylpiperidin-1-yl)-4-iodobenzoic acid

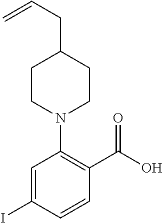 [SMILES: C=CCC1CCN(c2cc(I)ccc2C(=O)O)CC1]

Intermediate U was synthesized following the procedure for Intermediate C using methyl 2-fluoro-4-iodobenzoate in step 3.

### Intermediate V. 2⁵-iodo-5⁵-(1H-pyrazol-1-yl)-4,6-diaza-5(2,6)-pyridina-1(1,4)-piperidina-2(1,2)-benzenacycloundecaphan-9-en-3-one

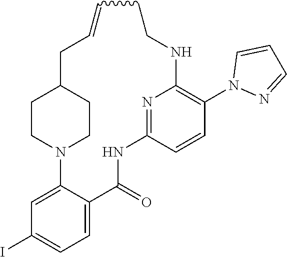 [SMILES: O=C1Nc2ccc(-n3cccn3)c(n2)NCCC=CCC2CCN(CC2)c2cc(I)ccc21]

Intermediate V was synthesized following the procedure for Intermediate F using Intermediate U in step 1

### Intermediate W. 3-bromo-N²-(pent-4-en-1-yl)pyridine-2,6-diamine

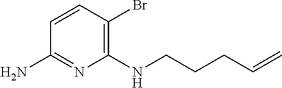

Intermediate W was synthesized following the procedure for Intermediate A using pent-4-en-1-amine as the nucleophile.

### Intermediate X. N²-(pent-4-en-1-yl)-3-(1H-pyrazol-1-yl)pyridine-2,6-diamine

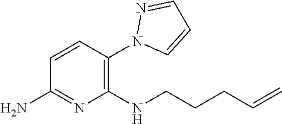 [SMILES: C=CCCCNc1nc(N)ccc1-n1cccn1]

Intermediate X was synthesized following the procedure for Intermediate B using Intermediate W as the starting substrate.

### Intermediate Y. methyl 3-((14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)thio)propanoate

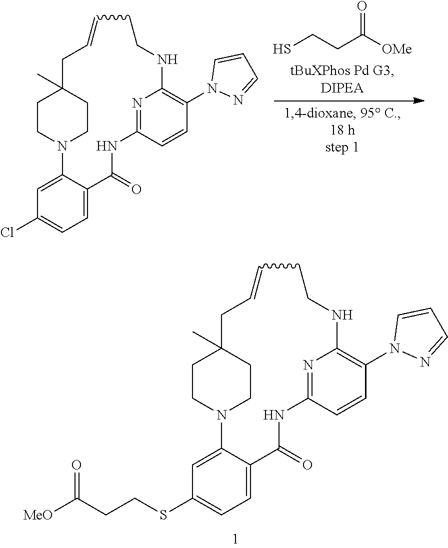

To a flask was added 2⁵-chloro-14-methyl-5⁵-(1H-pyrazol-1-yl)-4,6-diaza-5(2,6)-pyridina-1(1,4)-piperidina-2(1,2)-benzenacycloundecaphan-9-en-3-one (4.0 g, 8.4 mmol), t-BuXPhos Pd G3 (0.34 g, 0.42 mmol), 1,4-Dioxane (40 mL), Propanoic acid, 3-mercapto-, methyl ester (1.2 g, 1.1 mL, 10 mmol), and DIPEA (1.6 g, 2.2 mL, 13 mmol) under a N₂ atmosphere. The reaction mixture was then heated to 95° C. After 18 hours, reaction mixture was cooled to rt. The reaction mixture was then diluted with 1M aq. NaOH and extracted with EtOAc (3×). The combined organic layers were washed with brine, dried over MgSO₄, and concentrated. Purification via Isco column chromatography (0-100% EtOAc on heptane) provided methyl 3-((14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)thio)propanoate (3.41 g, 73% yield). LCMS (ESI-MS) m/z=561.2 [M+H]⁺.

### Intermediate Z. 2⁵-mercapto-1⁴-methyl-5⁵-(1H-pyrazol-1-yl)-4,6-diaza-5(2,6)-pyridina-1(1,4)-piperidina-2(1,2)-benzenacycloundecaphan-9-en-3-one

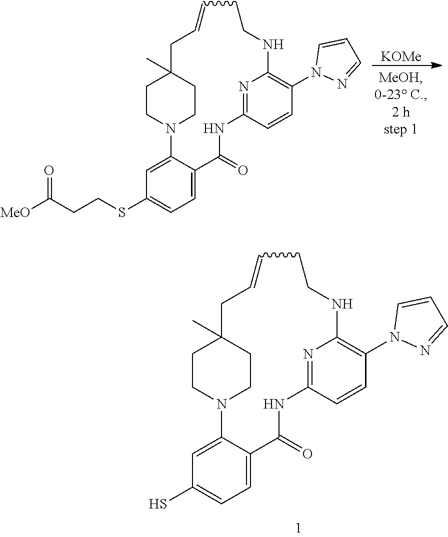 [SMILES: COC(=O)CCSc1ccc2c(c1)N1CCC(C)(CC=CCCNc3nc(ccc3-n3cccn3)NC2=O)CC1 ; CC12CC=CCCNc3nc(ccc3-n3cccn3)NC(=O)c3ccc(S)cc3N(CC1)CC2 ; "KOMe"]

To methyl 3-((14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)thio)propanoate (3.31 g, 5.90 mmol) in DMSO (60 mL) at 0° C. was added slowly Potassium methoxide (0.5 M in MeOH) (455 mg, 13.0 mL, 0.5 molar, 6.49 mmol). The reaction mixture was then warmed to rt. After 2 hour the reaction was quenched with 1M HCl and extracted with EtOAc (3×). The combined organic layers were washed with brine, dried over MgSO₄, and concentrated. Purification via Isco column chromatography (0-50% EtOAc on heptane) provided 2⁵-mercapto-14-methyl-5⁵-(1H-pyrazol-1-yl)-4,6-diaza-5(2,6)-pyridina-1(1,4)-piperidina-2(1,2)-benzenacycloundecaphan-9-en-3-one (2.61 g, 93% yield). LCMS (ESI-MS) m/z=475.2 [M+H]⁺.

### Intermediate AA: sodium (E)-5⁵-bromo-3-oxo-4,6-diaza-5(2,6)-pyridina-1(1,4)-piperidina-2(1,2)-benzenacycloundecaphan-9-ene-2⁵-sulfinate

### Step 1: tert-butyl 2-(4-allylpiperidin-1-yl)-4-iodobenzoate

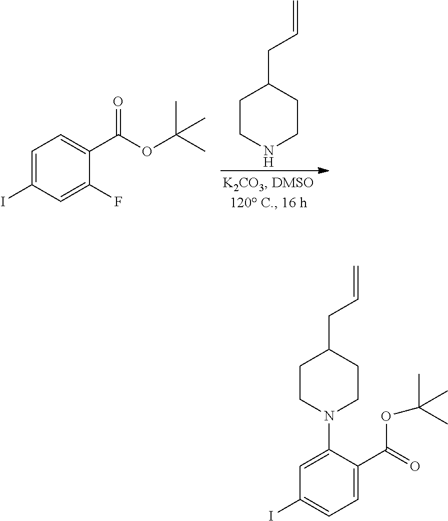

4-allylpiperidine (12 g, 93 mmol) and K₂CO₃ (19 g, 140 mmol) were added tert-butyl 2-fluoro-4-iodobenzoate (15 g, 47 mmol) in DMSO (150 mL) at room temperature. The resulting mixture was stirred at 120° C. for 16 hours. The resulting mixture was extracted with EA (3×100 mL). The combined organic layers were washed with water (300 mL), dried over anhydrous Na₂SO₄, filtered and concentrated under reduced pressure. The residue was purified by silica gel column chromatography, eluted with PE/EA (1:1) to afford tert-butyl 2-(4-allylpiperidin-1-yl)-4-iodobenzoate (17 g, 77% yield). LCMS (ESI-MS) m/z=428.1 [M+H]⁺.

### Step 2: tert-butyl 2-(4-allylpiperidin-1-yl)-4-((3-methoxy-3-oxopropyl)sulfonyl)benzoate

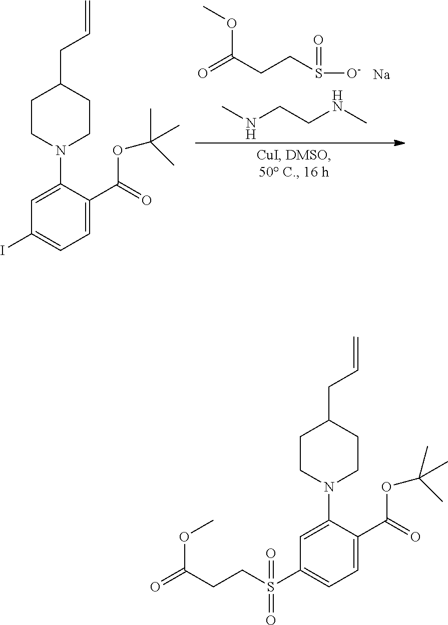

A solution of tert-butyl 2-(4-allylpiperidin-1-yl)-4-iodobenzoate (7 g, 20.0 mmol) and N¹,N²-dimethylethane-1,2-diamine (100 mg, 2.0 mmol), sodium 3-methoxy-3-oxopropane-1-sulfinate (4 g, 20.0 mmol) and CuI (300 mg, 2.0 mmol) in DMSO (70 mL) was stirred at 50° C. for 16 hours under nitrogen atmosphere. The resulting mixture was extracted with EA (3×50 mL). The combined organic layers were washed with water (3×50 mL), dried over anhydrous Na₂SO₄, filtered, and concentrated under reduced pressure. The residue was purified by silica gel column chromatography, eluted with PE/EA (5:1) to afford tert-butyl 2-(4-allylpiperidin-1-yl)-4-((3-methoxy-3-oxopropyl)sulfonyl)benzoate (5.5 g, 60% yield). LCMS (ESI-MS) m/z=452.3 [M+H]⁺.

### Step 3: 2-(4-allylpiperidin-1-yl)-4-((3-methoxy-3-oxopropyl)sulfonyl)benzoic acid

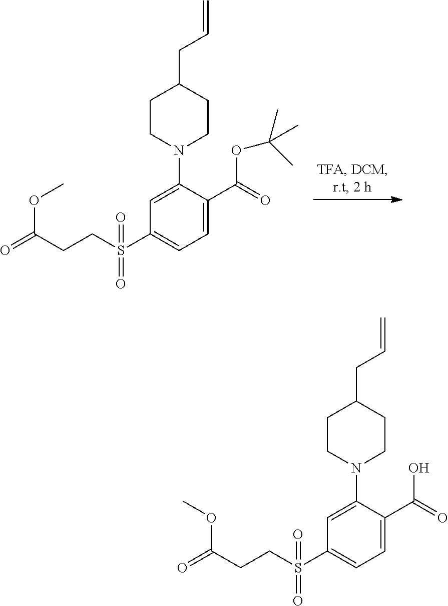 [SMILES: C=CCC1CCN(c2cc(S(=O)(=O)CCC(=O)OC)ccc2C(=O)OC(C)(C)C)CC1 ; C=CCC1CCN(c2cc(S(=O)(=O)CCC(=O)OC)ccc2C(=O)O)CC1]

Into a 250 mL round-bottom flask were added tert-butyl 2-(4-allylpiperidin-1-yl)-4-((3-methoxy-3-oxopropyl)sulfonyl)benzoate (7 g, 20.0 mmol) in DCM (80 mL) and TFA (16 mL) at 25° C. The solution reaction product 2 hours. The resulting mixture was concentrated under reduced pressure. The crude product 2-(4-allylpiperidin-1-yl)-4-((3-methoxy-3-oxopropyl)sulfonyl)benzoic acid (5.5 g, 70% yield) was used in the next step directly without further purification. LCMS (ESI-MS) m/z=396.1 [M+H]⁺.

### Step 4: Methyl 3-((3-(4-allylpiperidin-1-yl)-4-((5-bromo-6-(but-3-en-1-ylamino)pyridin-2-yl)carbamoyl)phenyl)sulfonyl)propanoate

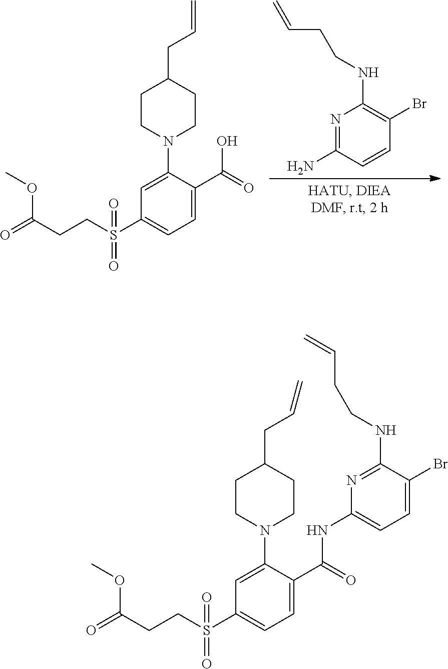 [SMILES: C=CCC1CCN(c2cc(S(=O)(=O)CCC(=O)OC)ccc2C(=O)O)CC1 ; C=CCCNc1nc(N)ccc1Br ; C=CCCNc1nc(NC(=O)c2ccc(S(=O)(=O)CCC(=O)OC)cc2N2CCC(CC=C)CC2)ccc1Br]

Into a 250 mL round-bottom flask were added 2-(4-allylpiperidin-1-yl)-4-((3-methoxy-3-oxopropyl)sulfonyl)benzoic acid (5.5 g, 4.0 mmol) in DMF (55 mL) and 3-bromo-N²-(but-3-en-1-yl)pyridine-2,6-diamine (5.1 g, 21.0 mmol), DIEA (7.3 mL) and HATU (7.9 g, 21 mmol) at room temperature. The mixture was stirred at room temperature for 2 hours. The resulting mixture was quenched by addition of water (50 mL) and extracted with EA (3×50 mL). The combined organic layers were washed with brine (3×50 mL), dried with anhydrous Na₂SO₄, filtered and concentrated under vacuum, and the residue was purified by silica gel column chromatography, eluted with PE/EA (1:1) to afford the desired product methyl 3-((3-(4-allylpiperidin-1-yl)-4-((5-bromo-6-(but-3-en−1-ylamino)pyridin-2-yl)carbamoyl)phenyl)sulfonyl)propanoate (10 g, 93% yield). LCMS (ESI-MS) m/z=621.15 [M+H]⁺.

### Step 5: Methyl (E)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate & methyl (Z)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate

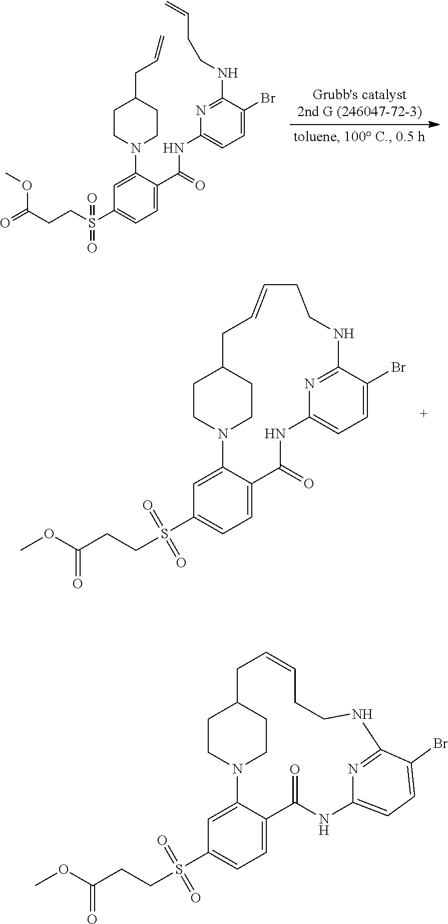 [SMILES: C=CCCNc1nc(NC(=O)c2ccc(S(=O)(=O)CCC(=O)OC)cc2N2CCC(CC=C)CC2)ccc1Br ; COC(=O)CCS(=O)(=O)c1ccc2c(c1)N1CCC(C/C=C/CCNc3nc(ccc3Br)NC2=O)CC1 ; COC(=O)CCS(=O)(=O)c1ccc2c(c1)N1CCC(C/C=C\CCNc3nc(ccc3Br)NC2=O)CC1]

A mixture of methyl 3-((3-(4-allylpiperidin-1-yl)-4-((5-bromo-6-(but-3-en-1-ylamino)pyridin-2-yl)carbamoyl)phenyl)sulfonyl)propanoate (10 g, 16 mmol) and Benzylidene(dichloro)(1,3-dimesityl-2-imidazolidinylidene)ruthenium-tricyclohexylphosphine (1:1) (1.4 g, 1.60 mmol) in toluene (200 mL) was stirred at 100° C. for 0.5 hour under nitrogen atmosphere. The resulting mixture was concentrated under vacuum. The residue was purified by silica gel column chromatography, eluted with PE/EA (5:1) to afford the mixture of -trans &-cis (6 g, purity 85%). The crude product (6 g) was re-purified by Prep-SFC with the conditions (Column: (S, S)-Whelk-O 1 4.6*50 mm, 3 μm; Mobile Phase B: ACN:MeOH=80: 20 (0.1% DEA); Flow rate: 4 mL/min; Gradient (B %): isocratic % B) to afford methyl (E)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate (2.8907 g, 4.85 mmol, 30% yield) and methyl (Z)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate (1.1837 g, 1.99 mmol, 12% yield).

### First Eluting Isomer: methyl (E)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate

¹H NMR (400 MHz, DMSO-d₆) δ 10.57 (s, 1H), 8.03 (d, J=8.1 Hz, 1H), 7.74 (d, J=8.4 Hz, 1H), 7.66-7.55 (m, 2H), 7.50 (d, J=8.4 Hz, 1H), 6.44 (d, J=6.5 Hz, 1H), 5.76 (s, 1H), 5.61-5.41 (m, 2H), 3.64 (t, J=7.2 Hz, 3H), 3.55 (s, 3H), 3.31-3.23 (m, 2H), 2.80 (d, J=11.5 Hz, 2H), 2.66 (d, J=7.3 Hz, 2H), 2.22 (s, 2H), 2.05 (s, 2H), 1.78-1.62 (m, 3H), 1.51 (d, J=12.0 Hz, 2H). LCMS (ESI-MS) m/z=593.05 [M+H]⁺.

### Step 6: Intermediate AA

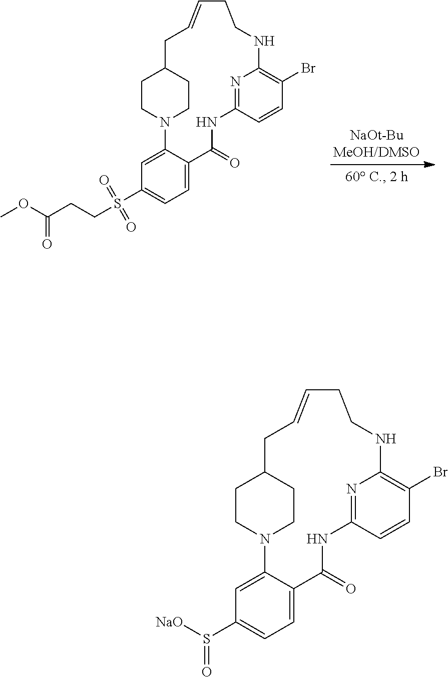

A 40 mL vial equipped with a magnetic stir bar was sequentially charged with a solution of sodium tert-butoxide (81.2 mg, 1.0 equiv, 845 μmol) in methanol (227 μL), methyl (E)-3-((5⁵-bromo-3-oxo-4,6-diaza-5(2,6)-pyridina-1(1,4)-piperidina-2(1,2)-benzenacycloundecaphan-9-en-2⁵-yl)sulfonyl)propanoate (500 mg, 1 Eq, 0.845 mmol), and DMSO (5 mL). The vial was capped, and the contents were allowed to stir at 60° C. for 2 h. After this time, the stir bar was removed and the reaction mixture was evaporated under reduced pressure to afford the desired product, sodium (E)-5⁵-bromo-3-oxo-4,6-diaza-5(2,6)-pyridina-1(1,4)-piperidina-2(1,2)-benzenacycloundecaphan-9-ene-2⁵-sulfinate (444 mg, 93% yield).

Intermediates AB and AC were synthesized using the procedure for Intermediate AA with 4-(allyloxy)-4-methylpiperidine used as the nucleophile in Step 1.

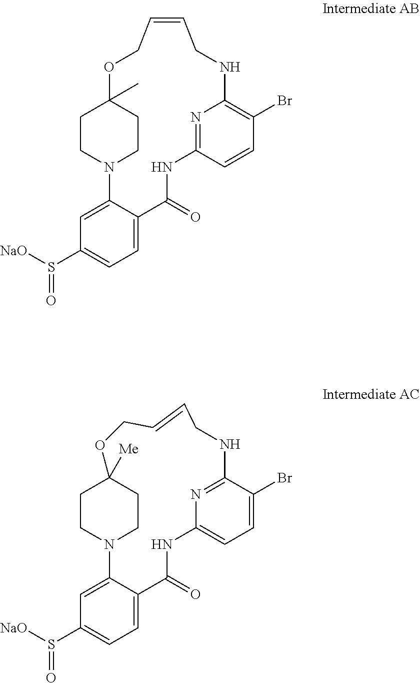

### General Procedures for High-Throughput, Plate-Based Syntheses

### General Procedure 1: Palladium-Catalyzed Cross-Coupling:

A 96-well Analytical Sales Paradox plate equipped with 8×30 1 mL vials was charged with amine or alcohol building blocks (1.5 eq). The plate was transferred to a nitrogen-filled glovebox and was equipped with stir bars. The plate was then sequentially charged with solutions in 1,4-dioxane of the aryl halide core (1.0 equiv, 30-50 mmol, limiting reagent), palladium precatalyst (0.1 equiv), and base (4.0 equiv). The plate was sealed, removed from the glovebox, transferred to a heating block attached to a Tumble Stirring device, and was heated to 100° C. for 16 h while stirring at 1000 RPM. The reaction plate was allowed to cool to 25° C. Reaction mixtures were evaporated under vacuum, subsequently diluted with DMSO (500 uL), and filtered through a 96-well glass filter plate into a collection plate. The reactions were further diluted with MeOH:DMSO (1:1, 1500 uL total volume). The crude reactions in the plate were subjected to mass-directed purification to afford the desired products.

C—N Cross-Coupling: Amide- and Sulfonamide-like Nucleophiles: Employed t-BuXPhos Pd G3 as the precatalyst and cesium carbonate as the base.

C—N Cross-Coupling: Alkyl Amine Nucleophiles: Employed t-BuXPhos Pd G3 as the precatalyst and sodium tert-butoxide as the base.

C—O Cross-Coupling: Alcohol Nucleophiles: Employed t-BuBrettPhos Pd G3 as the precatalyst and sodium tert-butoxide as the base.

C—C Cross-Coupling: Alkyl trifluoroborate nucleophiles: Employed RuPhos Pd G3 as the precatalyst and cesium carbonate as the base.

### General Procedure 2: Copper-Catalyzed Cross-Coupling

A 96-well Analytical Sales Paradox plate equipped with 8×30 1 mL vials was charged with heteroaryl amine building blocks (1.5 eq). The plate was transferred to a nitrogen-filled glovebox and was equipped with stir bars. The plate was then sequentially charged with solutions in DMSO of the aryl halide core (1.0 equiv, 30-50 mmol, limiting reagent), ligand (0.1 equiv) and copper (I) iodide (0.1 equiv), and sodium tert-butoxide (4.0 equiv). The plate was sealed, removed from the glovebox, transferred to a heating block attached to a Tumble Stirring device, and was heated at 100° C. for 16 h while stirring at 1000 RPM. After this time, the reaction plate was allowed to cool to 25° C. Reaction mixtures were filtered through a 96-well glass filter plate into a collection plate. The reactions were further diluted with MeOH:DMSO (1:1, 1500 uL total volume). The crude reactions in the plate were subjected to mass-directed purification to afford the desired products.

trans-C-linked Et-Sulfones and Et-Sulfonamides with electron-rich and -poor pyrazoles: Employed DPEO (N,N′-Bis(2-phenylethyl)ethanediamide) as the ligand.

trans-O-linked Et-Sulfones with electron-rich pyrazoles: Employed BTMO (N,N′-Bis(furan-2-ylmethyl)oxalamide) as the ligand.

cis-O-linked Et-Sulfonamides with electron-rich pyrazoles: Employed BNMO (N¹,N²-Bis(1-naphthalenylmethyl)ethanediamide) as the ligand.

cis-O-linked Et-Sulfones and Et-Sulfonamides with electron-poor pyrazoles: Employed BNMO (N¹,N²-Bis(1-naphthalenylmethyl)ethanediamide) as the ligand.

### Preparative Mass-Directed Auto Purification (MDAP):

High-Pressure Liquid Chromotagraphy (Reverse Phase): Preparative Mass Directed Auto Purification (MDAP) was conducted on an Agilent 1290 Infinity II LCMS equipped with fraction collector. A YMC Triart Column (150 mm×20 mm, 120 Å, 5 μm) was used to run a gradient of 20-70% MeCN with 0.1% v/v TFA or 30-65% MeCN with 0.1% v/v NH₄OAc at 25-40° C. and 50 mL/min flow rate. UV detection was monitored at 254 nm and 280 nm. Mass spectrometry detection was monitored using a single quadrupole Agilent LC/MSD with positive-negative polarity switching in ESI mode.

Supercritical Fluid Chromatography: Preparative Mass Directed Auto Purification (MDAP) was also conducted on a Shimadzu SFC-MS (LC-20AP, SFC-40P, SPD-40) equipped with fraction collector. Four different Princeton columns were used to adjust to sample chromatographic profile: 2-EthylPyridine (21×150 mm, 60 Å, 5 μm), diol (21×150 mm, 60 Å, 5 μm), silica (21×150 mm, 60 Å, 5 μm), and methane sulfonamide (21×150 mm, 60 Å, 5 μm). Samples were run at a gradient 5-50% MeOH with 10 mM Ammonia with respect to CO2 at 80 mL/min over 5 min at 50° C.

The purity of each compound was determined on an Agilent 1290 Infinity II LCMS. A HALO C18 column (1.5 mm×50 mm, 90 Λ, 2.7 μm) was used to run a gradient of 10-98% MeCN with 0.1% v/v TFA at 40° C. and 0.7 mL/min flow rate. UV detection was monitored at 254 nm and 280 nm. Mass spectrometry detection was monitored using a single quadrupole Agilent LC/MSD with positive-negative polarity switching in ESI mode.

### Compounds of the Invention

### Compounds 1 and 2.

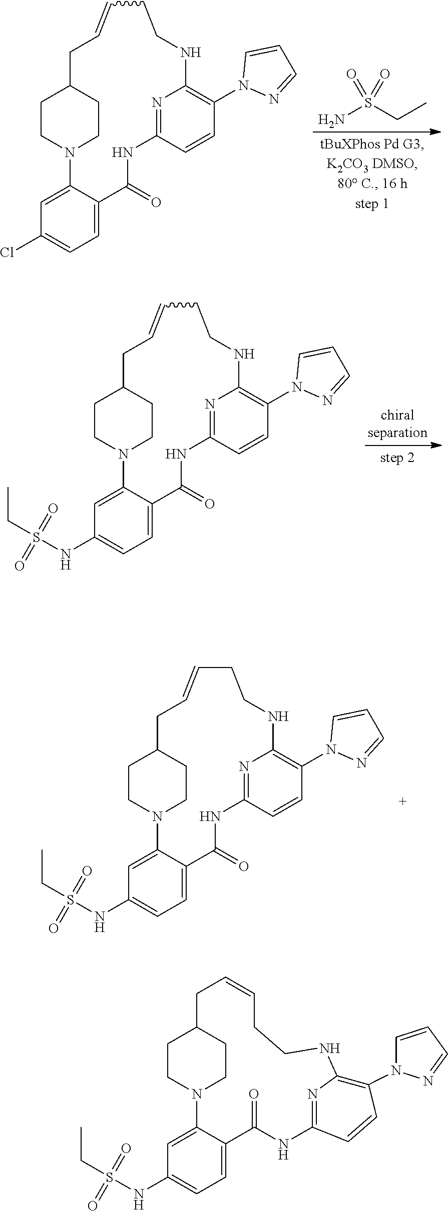

Step 1. N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide: t-BuXphos Pd G3 (410 mg, 0.39 mmol) was added to a mixture of 2⁵-chloro-5⁵-(1H-pyrazol-1-yl)-4,6-diaza-5(2,6)-pyridina-1(1,4)-piperidina-2(1,2)-benzenacycloundecaphan-9-en-3-one (900 mg, 1.94 mmol) and ethanesulfonamide (636 mg, 5.83 mmol), K₂CO₃ (806 mg, 5.83 mmol) in DMSO (10 mL) under nitrogen atmosphere. The mixture was stirred at 80° C. for 2 hours. The reaction mixture was diluted with water (10 mL). The resulting mixture was extracted with EA (2×10 mL). The combined organic layers were washed with brine (100 mL), dried over anhydrous Na₂SO₄. After filtration, the filtrate was concentrated under reduced pressure. The residue was purified by silica gel column chromatography, eluted with PE:EA=1:1 to afford N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (500 mg, 47.9% yield).

Step 2. Compounds 1 and 2: N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (500 mg, 0.93 mmol) was dissolved with MeOH and purified by Chiral-Prep-HPLC with the following conditions: Column: (S, S)-WHELK-O1 3*25 cm, 5 um; Mobile Phase A: CO₂, Mobile Phase B: DCM:MeOH=1:1(0.1% 2M NH3-MEOH); Flow rate: 90 mL/min; Gradient: isocratic 40% B; Column Temperature(° C.): 35; Back Pressure(bar): 100; Wave Length: 220 nm; RT1 (min): 9.7; RT2 (min): 34.35; Sample Solvent: MEOH; Injection Volume: 6 mL; Number Of Runs: 2 to afford:

First eluting isomer (Compound 1): (E)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (300.4 mg, 28.7% yield).

Second eluting isomer (Compound 2): (Z)—N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (17.1 mg, 1.6% yield).

### Compound 3.

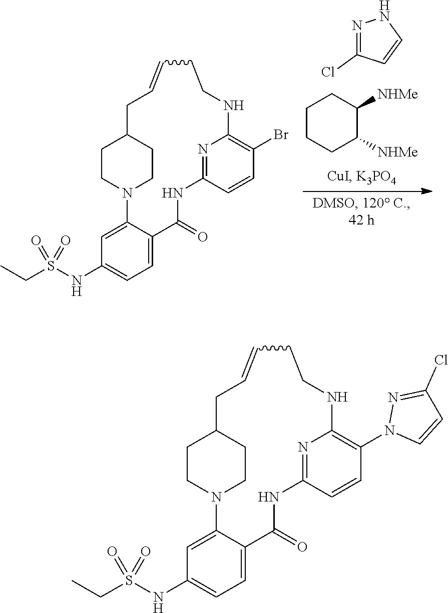

To a vial was added N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (23 mg, 42 μmol), 3-chloro-1H-pyrazole (13 mg, 0.13 mmol), CuI (8.0 mg, 42 μmol), (1R)—N1,N2-dimethylcyclohexane-1,2-diamine (6.0 mg, 42 μmol), and DMSO (0.20 mL). The reaction mixture was then heated to 120° C. After 42 h, the reaction mixture was cooled to rt, filtered, and purified via preparative HPLC (10-100% MeCN in H₂O with 0.1% TFA) followed by lyophilization to provide N-(5⁵-(3-chloro-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (10.6 mg, 44% yield).

### Compound 4.

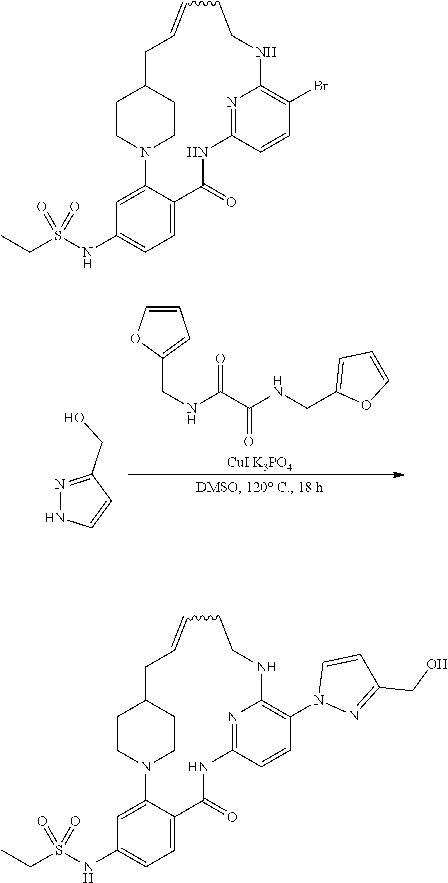

To a vial was added N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (25 mg, 46 μmol), (1H-Pyrazol-5-yl)methanol (6.7 mg, 68 μmol), CuI (1.7 mg, 9.1 μmol), K₃PO₄ (39 mg, 15 μL, 0.18 mmol), N¹,N²-bis(furan-2-ylmethyl)oxalamide (4.5 mg, 18 μmol) and DMSO (0.5 mL) under a N₂ atmosphere. The reaction mixture was then heated to 120° C. After 18 hour, the reaction mixture was diluted with water and extracted with DCM (3×). The combined organic layers were washed with brine, dried over MgSO₄, and concentrated. Purification via Isco column chromatography (0-40% EtOAc in heptane) provided N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (15.7 mg, 61% yield).

### Compounds 5 and 6.

N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (40 mg, 71 μmol) was dissolved in MeOH (2 mL) and purified by Chiral-Prep-HPLC with the following conditions: Column: (R, R)-WHELK-01-Kromasil, 2.12*25 cm, 5 μm; Mobile Phase A: Hex(0.5% 2M NH3-MeOH)--HPLC, Mobile Phase B: MeOH:DCM=1:1--HPLC; Flow rate: 20 ML/MIN mL/min; Gradient: isocratic 70; Wave Length: 254/220 nm nm; RT1 (min): 4.521; RT2 (min): 9.514; Sample Solvent: EtOH--HPLC; Injection Volume: 1.0 mL; Number Of Runs: 2. The fractions were collected, evaporated and lyophilized to afford:

First eluting isomer (Compound 5) (E)-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (17.8 mg, 45% yield).

Second eluting isomer (Compound 6) (Z)—N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.5 mg, 3.8% yield).

Compound 7 was synthesized using the procedure from Compound 4 except 3-(methylsulfonyl)-1H-pyrazole was used to provide N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (50 mg, 86% yield).

### Compounds 8 and 9.

N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (50 mg) was dissolved with MeOH, DCM and purified by Chiral-Prep-HPLC with the following conditions: Column: (R, R); Mobile Phase A: Hex(0.1% DEA): (MeOH:DCM=1:1)=20:80; Flow rate: 1 mL/min; Gradient: isocratic; Injection Volume: 2 μl mL to afford:

First eluting isomer (Compound 8): (E)-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (24.0 mg, 48% yield).

Second eluting isomer (Compound 9): (Z)—N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.9 mg, 7.8% yield).

Compound 10 as prepared according to the procedure outlined for Compound 4 except 3-cyanopyrazole and 8-Quinolinol-N-oxide were used, and the reaction was run for 70 h, to provide N-(5⁵-(3-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (59 mg, 57% yield).

### Compounds 11 and 12.

N-(5⁵-(3-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (78 mg, 0.14 mmol) was dissolved in MeOH (3 mL) and purified by Chiral-Prep-HPLC with the following conditions: Column: Mobile Phase A: Hex(0.1% DEA): (MeOH:DCM=1:1)=40:60; Flow rate: 1 mL/min; Gradient: isocratic; Injection Volume: 0.5 μl mL to afford:

First eluting isomer (Compound 11): (E)-N-(5⁵-(3-cyano-1H-pyrazol-1-yl)-3-oxo-4,6-diaza-5(2,6)-pyridina-1(1,4)-piperidina-2(1,2)-benzenacycloundecaphan-9-en-2⁵-yl) ethanesulfonamide (25 mg, 32% yield).

Second eluting isomer (Compound 12): (Z)—N-(5⁵-(3-cyano-1H-pyrazol-1-yl)-3-oxo-4,6-diaza-5(2,6)-pyridina-1(1,4)-piperidina-2(1,2)-benzenacycloundecaphan-9-en-2⁵-yl) ethanesulfonamide (5.9 mg, 7.5% yield).

### Compound 13. N-(5⁵-(3-methoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (Compound 13)

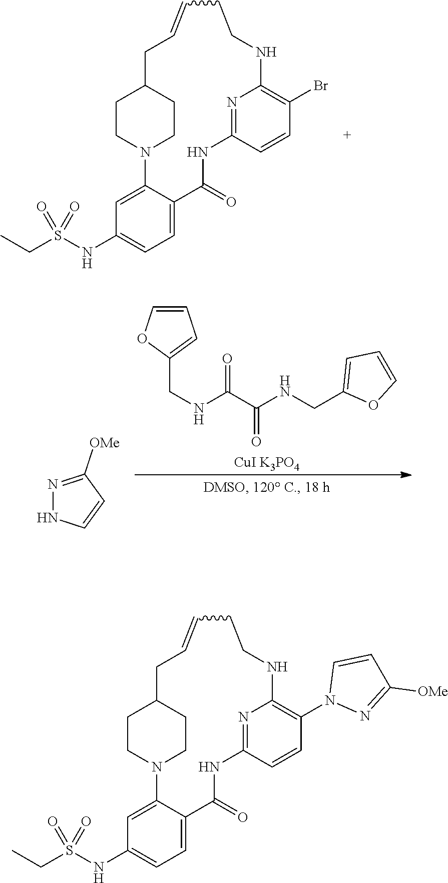

The procedure from Compound 4 was followed except 3-methoxy-1H-pyrazole was used to provide N-(5⁵-(3-methoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (25.4 mg, 61% yield).

### Compounds 14 and 15:

N-(5⁵-(3-methoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (28 mg) was separated by Prep-Chiral-HPLC with the following conditions: =Column: (R, R)1; Mobile Phase A: Hex(0.1% DEA): (MeOH:DCM=1:1)=20:80; Flow rate: 1 L/mmin; Gradient: isocratic; Injection Volume: 2 μl mL to afford:

First eluting isomer (Compound 14): (E)-N-(5⁵-(3-methoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (13.8 mg, 49% yield).

Second eluting isomer (Compound 15): (Z)—N-(5⁵-(3-methoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.7 mg, 9.6% yield).

Compound 16 was prepared according to the procedure outlined for Compound 4 except 4-cyano-1H-pyrazole and 8-Quinolinol-N-oxide were used to provide N-(5⁵-(4-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (25 mg, 46% yield).

Compounds 17 and 18 were purified via Chiral-Prep-HPLC following the procedure from Compound 5 to afford (E)-N-(5⁵-(4-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (Compound 17) (Z)—N-(5⁵-(4-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (Compound 18).

Compound 19 was prepared according to the procedure outlined for Compound 16 was followed to provide (1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide (15 mg, 27% yield).

### Compounds 20 and 21.

(E)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide (13.1 mg) was dissolved in MeOH (2 mL) and separated by Prep-Chiral HPLC with the following conditions (Column: (R, R)-WHELK-01-Kromasil, 2.12*25 cm, 5 μm; Mobile Phase A: Hex--HPLC, Mobile Phase B: MeOH:DCM=1:1-HPLC; Flow rate: 20 mL/min; Gradient (B %): isocratic 70; Wave Length: 254/220 nm nm; RT1 (min): 5.237; RT2 (min): 9.076; Sample Solvent: EtOH--HPLC; Injection Volume: 2.0 mL) to afford:

First eluting isomer (Compound 20): (E)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide (1.7 mg, 13% yield).

Second eluting isomer (Compound 21): (Z)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide (0.2 mg, 1.5% yield).

Compound 22 was prepared according to the procedure outlined for Compound 4 was followed except 1H-pyrazol-4-ylmethanol and 8-Quinolinol-N-oxide were used to provide N-(5⁵-(4-(hy droxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (9.0 mg, 20% yield).

### Compounds 23 and 24.

N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (13.085 mg) was separated by Prep-Chiral-HPLC with the following conditions (Column: (R, R)-WHELK-01-Kromasil, 2.12*25 cm, 5 μm; Mobile Phase A: Hex--HPLC, MobilePhase B: MeOH:DCM=1:1--HPLC; Flow rate: 20 ML/MIN mL/min; Gradient: isocratic 70; Wave Length: 254/220 nm nm; RT1 (min): 4.625; RT2 (min): 8.16; Sample Solvent: EtOH--HPLC; Injection Volume: 2.0 mL) to afford:

First eluting isomer (Compound 23): (E)-N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.1 mg, 31% yield).

Second eluting isomer (Compound 24): (Z)—N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (0.3 mg, 7.6% yield).

### Compounds 25 and 26.

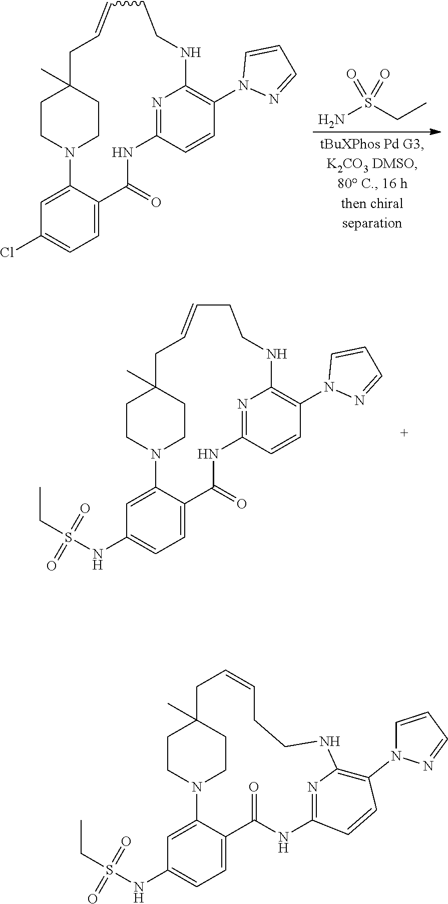

Into a solution of 2⁵-chloro-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (110 mg, 0.231 mmol) in DMSO (5 mL) was added ethanesulfonamide (75.50 mg, 0.692 mmol), t-BuXPhos Pd G3 (36.6 mg, 0.046 mmol) and K₂CO₃ (95.6 mg, 0.693 mmol). The resulting solution was stirred at 80° C. for 16 hours. After cooling to room temperature, the reaction mixture was diluted with water (10 mL), extracted with EA (3×10 mL). The combined organic layers were washed with brine (2×10 mL), dried with anhydrous Na₂SO₄, filtered and evaporated. The residue was purified with Chiral-HPLC by the following conditions: Column: CHIRAL ART Cellulose-SC, 2*25 cm, 5 μm; Mobile Phase A: Hex(0.5% 2M NH3-MeOH)--HPLC, Mobile Phase B: MeOH:DCM=1:1--HPLC; Flow rate: 20 ML/MIN mL/min; Gradient: isocratic 40; Wave Length: 254/220 nm nm; RT1 (min): 7.495; RT2 (min): 9.27; Sample Solvent: EtOH--HPLC; Injection Volume: 0.6 mL; Number Of Runs: 16. The fractions were collected, evaporated and lyophilized to afford:

First eluting isomer (Compound 25): (E)-N-(1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (43.6 mg, 34.4% yield).

Second eluting isomer (Compound 26) (Z)—N-(1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethanesulfonamide (10.8 mg, 8.5% yield). Compounds 27 and 28.

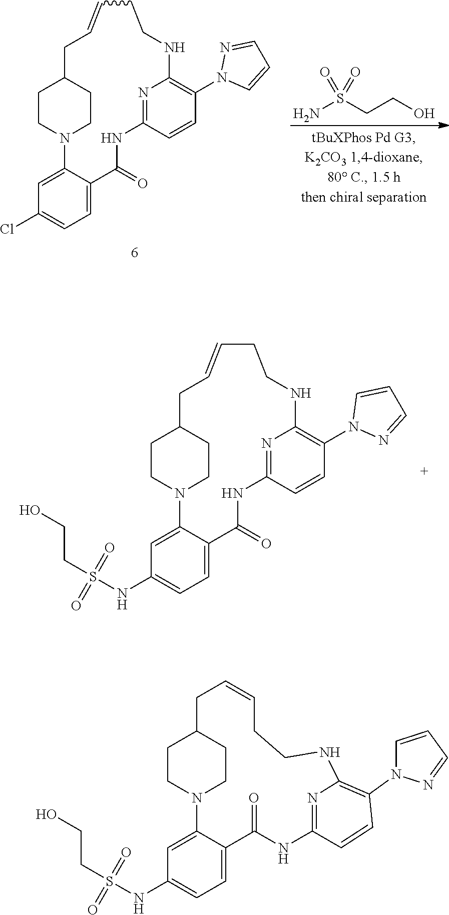 [SMILES: O=C1Nc2ccc(-n3cccn3)c(n2)NCCC=CCC2CCN(CC2)c2cc(Cl)ccc21 ; NS(=O)(=O)CCO ; O=C1Nc2ccc(-n3cccn3)c(n2)NCC/C=C/CC2CCN(CC2)c2cc(NS(=O)(=O)CCO)ccc21 ; O=C1Nc2ccc(-n3cccn3)c(n2)NCC/C=C\CC2CCN(CC2)c2cc(NS(=O)(=O)CCO)ccc21]

A mixture of 2⁵-chloro-5⁵-(1H-pyrazol-1-yl)-4,6-diaza-5(2,6)-pyridina-1(1,4)-piperidina-2(1,2)-benzenacycloundecaphan-9-en-3-one (83 mg, 0.18 mmol), t-BuXPhos Pd G3 (14 mg, 18 μmol), 2-hydroxyethane-1-sulfonamide (0.17 g, 1.3 mmol), and K₂CO₃ (74 mg, 0.54 mmol) was placed under a nitrogen atmosphere and 1,4-dioxane (2.0 mL) was added. The reaction mixture was then heated to 80° C. After 1.5 hour, the reaction mixture was cooled to rt, diluted with EtOAc, filtered through celite, and concentrated. Purification via Isco column chromatography (0-50% EtOAc in heptane) followed by purification via preparative HPLC (10-100% MeCN in H₂O with 0.1% TFA) and lyophilization provided 2-hydroxy-N-(3-oxo-5⁵-(1H-pyrazol-1-yl)-4,6-diaza-5(2,6)-pyridina-1(1,4)-piperidina-2(1,2)-benzenacycloundecaphan-9-en-25-yl)ethane-1-sulfonamide (35.1 mg, 28% yield). LCMS (ESI-MS) m/z=552.2 [M+H]⁺.

The product was dissolved in MeOH (50 mL) and purified with the following conditions: Column: CHIRALPAK IG-3 3.0*50 mm, 3 um; Mobile Phase B:MeOH: ACN=1:1(0.2% 7M NH3-MeOH); Flow rate: 2 mL/min; Gradient: isocratic % B to provide:

First eluting isomer (Compound 27): (E)-2-hydroxy-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl) ethane-1-sulfonamide (18.8 mg, 53.5% yield). LCMS (ESI-MS) m/z=552.3 [M+H]⁺.

Second eluting isomer (Compound 28): (Z)-2-hydroxy-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl) ethane-1-sulfonamide (4.0 mg, 11.3% yield). LCMS (ESI-MS) m/z=552.3 [M+H]⁺.

### Compounds 29 and 30.

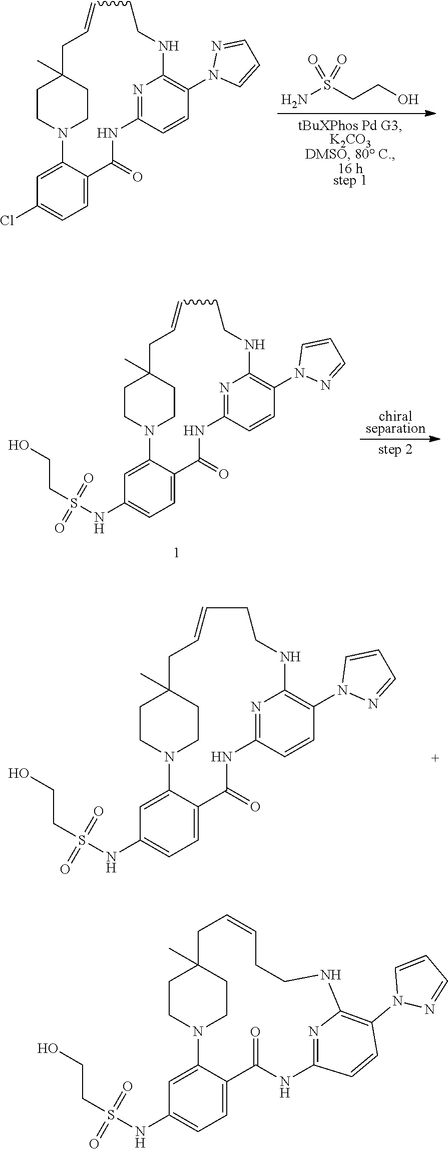

Step 1. 2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide: Into a solution of 2⁵-chloro-14-methyl-5⁵-(1H-pyrazol-1-yl)-4,6-diaza-5(2,6)-pyridina-1(1,4)-piperidina-2(1,2)-benzenacycloundecaphan-9-en-3-one (4 g, 8 mmol) in DMSO (40 mL) was added 2-hydroxyethane-1-sulfonamide (3 g, 0.03 mol), t-BuXPhos Pd G3 (1 g, 2 mmol) and K₂CO₃ (3 g, 0.03 mol). The resulting solution was stirred at 80° C. for 16 hours. After cooling to room temperature, the reaction mixture was diluted with water (80 mL) and extracted with EA (3×80 mL). The combined organic layers were washed with brine (2×80 mL), dried with anhydrous Na₂SO₄, filtered and evaporated to give a crude. The crude was purified with silica gel column, eluted with EA:PE=1:1ito afford 2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (2.5 g, 50% yield). LCMS (ESJ-MS) m/z=566.3 [M+H]⁺.

Step 2. Compounds 29 and 30: 2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (2.5 g, 4.4 mmol) was dissolved in DCM (100 mL), DMSO (100 mL) and formic acid (100 mL) and purified with the following conditions: Column: (R, R)-WHELK-01-Kromasil, 2.12*25 cm, 5 μm; Mobile Phase A: Hex(0.5% 2M NH₃-MeOH)--HPLC, Mobile Phase B: MeoH:DCM=1:1--HPLC; Flow rate: 20 ML/MIN mL/min; Gradient: isocratic 70; Wave Length: 254/220 nm nm; RT1 (min): 4.521; RT2 (min): 9.514; Sample Solvent: EtOH--HPLC; Injection Volume: 1.0 mL; Number Of Runs: 5. The fractions were collected, evaporated and lyophilized to afford:

First eluting isomer: (E)-2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (1.08 g, 43% yield).

Second eluting isomer: (Z)-2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (235.2 mg, 9.3% yield).

### Compounds 31 and 32.

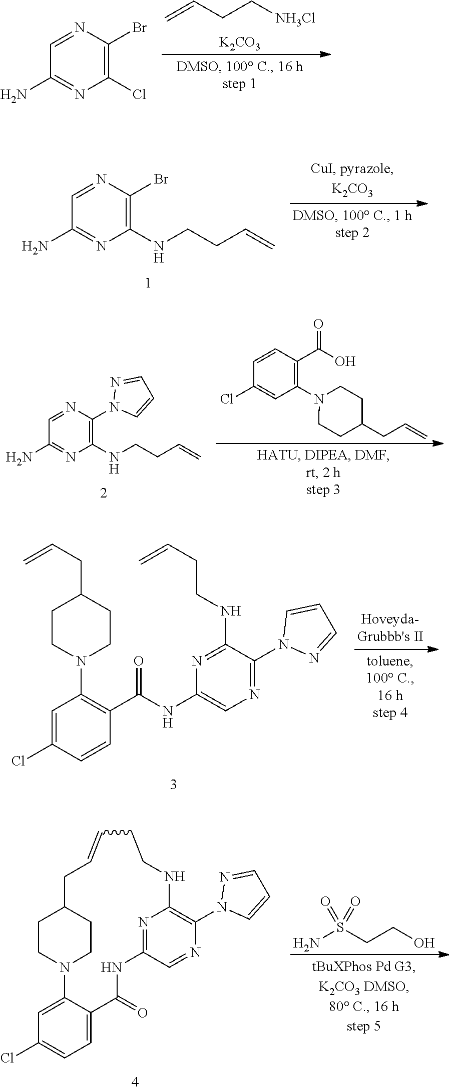

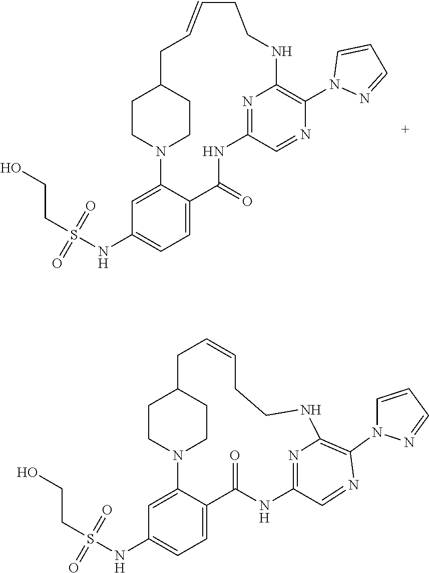 [SMILES: O=C1Nc2cnc(-n3cccn3)c(n2)NCC/C=C/CC2CCN(CC2)c2cc(NS(=O)(=O)CCO)ccc21 ; O=C1Nc2cnc(-n3cccn3)c(n2)NCC/C=C\CC2CCN(CC2)c2cc(NS(=O)(=O)CCO)ccc21]

Step 1. 3-bromo-N²-(but-3-en-1-yl)pyrazine-2,6-diamine: Into a solution of 5-bromo-6-chloropyrazin-2-amine (2 g, 0.01 mol) in DMSO (20 mL) was added but-3-en-1-amine, HCl (3 g, 0.03 mol) and K₂CO₃ (7 g, 0.05 mol). The resulting solution was stirred at 100° C. for 16 hour. After cooling to room temperature, the reaction mixture was diluted with water (30 mL), extracted with EA (30 mL×2). The organic layers were combined, washed with brine (30 mL×2), dried with anhydrous Na₂SO₄, filtered and concentrated. The crude was purified with silica gel column, eluted with EA:PE=1:1 to afford 3-bromo-N²-(but-3-en-1-yl)pyrazine-2,6-diamine (1 g, 40% yield). LCMS (ESI-MS) m/z=242.9 [M+H]⁺.

Step 2. N²-(but-3-en-1-yl)-3-(1H-pyrazol-1-yl)pyrazine-2,6-diamine: Into a solution of 3-bromo-N²-(but-3-en-1-yl)pyrazine-2,6-diamine (1 g, 4 mmol) in DMSO (10 mL) was added 1H-pyrazole (0.8 g, 0.01 mol), CuI (2 g, 0.01 mol), (1R,2R)—N¹,N₂-dimethylcyclohexane-1,2-diamine (2 g, 0.01 mol) and K₂CO₃ (2 g, 0.01 mol). The resulting solution was stirred at 100° C. for 16 hour. After cooling to room temperature, the reaction mixture was diluted with water (20 mL), extracted with EA (2×20 mL). The combined organic layers were combined, washed with brine (2×20 mL), dried with anhydrous Na₂SO₄, filtered and evaporated to give a crude. The crude was purified with silica gel column, eluted with EA:PE=1:1 to afford N²-(but-3-en-1-yl)-3-(1H-pyrazol-1-yl)pyrazine-2,6-diamine (600 mg, 60% yield). LCMS (ESI-MS) m/z=231.0 [M+H]⁺.

Step 3. 2-(4-allylpiperidin-1-yl)-N-(6-(but-3-en-1-ylamino)-5-(1H-pyrazol-1-yl)pyrazin-2-yl)-4-chlorobenzamide: Into a solution of N²-(but-3-en-1-yl)-3-(1H-pyrazol-1-yl)pyrazine-2,6-diamine (200 mg, 0.869 mmol) in DMF (5 mL) was added 2-(4-allylpiperidin-1-yl)-4-chlorobenzoic acid (243 mg, 0.869 mmol), DIEA (337 mg, 2.61 mmol) and HATU (495 mg, 1.30 mmol). The resulting solution was stirred at 60° C. for 16 hour. After cooling to room temperature, the reaction mixture was diluted with water (10 mL), extracted with EA (2×10 mL). The organic layers were combined, washed with brine (2×10 mL), dried with anhydrous Na2SO4, filtered and evaporated to give a crude. The crude was purified with silica gel column, eluted with EA:PE=1:4 to afford 2-(4-allylpiperidin-1-yl)-N-(6-(but-3-en-1-ylamino)-5-(1H-pyrazol-1-yl)pyrazin-2-yl)-4-chlorobenzamide (250 mg, 58% yield). LCMS (ESI-MS) m/z=492.2 [M+H]⁺.

Step 4. 2⁵-chloro-5⁵-(1H-pyrazol-1-yl)-4,6-diaza-5(2,6)-pyrazina-1(1,4)-piperidina-2(1,2)-benzenacycloundecaphan-9-en-3-one: Into a solution of 2-(4-allylpiperidin-1-yl)-N-(6-(but-3-en-1-ylamino)-5-(1H-pyrazol-1-yl)pyrazin-2-yl)-4-chlorobenzamide (240 mg, 0.488 mmol) in toluene (24 mL) was added Dichloro(1,3-dimesityl-2-imidazolidinylidene)(2-isopropoxybenzylidene)ruthenium (61.1 mg, 0.0976 mmol). The resulting solution was stirred at 100° C. for 16 hour. After cooling to room temperature, the reaction mixture was concentrated and purified via silica gel column chromatography, eluted with EA:PE=1:5 to afford 2⁵-chloro-5⁵-(1H-pyrazol-1-yl)-4,6-diaza-5(2,6)-pyrazina-1(1,4)-piperidina-2(1,2)-benzenacycloundecaphan-9-en-3-one (120 mg, 53.0% yield). LCMS (ESI-MS) m/z=464.2 [M+H]⁺.

Step 5. Compounds 31 and 32: Into a solution of 2⁵-chloro-5⁵-(1H-pyrazol-1-yl)-4,6-diaza-5(2,6)-pyrazina-1(1,4)-piperidina-2(1,2)-benzenacycloundecaphan-9-en-3-one (110 mg, 237 μmol) in DMSO (5 mL) was added 2-hydroxyethane-1-sulfonamide (89.0 mg, 711 μmol), K₂CO₃ (98.3 mg, 711 μmol) and t-BuXPhos Pd G3 (37.7 mg, 47.4 μmol). The resulting solution was stirred at 80° C. for 16 hour. After cooling to room temperature, the reaction mixture was diluted with water (10 mL), extracted with EA (2×10 mL). The organic layers were washed with brine (2×10 mL), dried with anhydrous Na₂SO₄, filtered and evaporated. The residue was purified by Prep-HPLC with the following conditions: Column: XBridge Prep OBD C18 Column, 30*150 mm, 5 μm; Mobile Phase A: Water(10 nmol/LNH4HCO3), Mobile Phase B: ACN; Flow rate: 60 mL/min mL/min; Gradient: 48% B to78% B in 10 min; Wavelength: 254 nm/220 nm nm; RT1 (min): 7.88. The fractions were collected, evaporated and lyophilized to afford:

First eluting isomer (Compound 31): (E)-2-hydroxy-N-(3-oxo-5⁵-(1H-pyrazol-1-yl)-4,6-diaza-5(2,6)-pyrazina-1(1,4)-piperidina-2(1,2)-benzenacycloundecaphan-9-en-2⁵-yl)ethane-1-sulfonamide (21.5 mg, 16.4% yield). LCMS (ESI-MS) m/z=553.3 [M+H]⁺.

Second eluting isomer (Compound 32): (Z)-2-hydroxy-N-(3-oxo-5⁵-(1H-pyrazol-1-yl)-4,6-diaza-5(2,6)-pyrazina-1(1,4)-piperidina-2(1,2)-benzenacycloundecaphan-9-en-2⁵-yl)ethane-1-sulfonamide (7.4 mg, 5.6% yield).

Compounds 33 and 34 were prepared according to the procedure outlined for Compounds 27 and 28 using 2-hydroxy-N-methylethane-1-sulfonamide and increasing the reaction time to 16 hours to afford (E)-2-hydroxy-N-methyl-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (Compound 33) and (Z)-2-hydroxy-N-methyl-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (Compound 34).

Compounds 35 and 36 were prepared according to the procedure outlined for Compounds 29 and 30 using 2-hydroxy-N-methylethane-1-sulfonamide in Step 1 to afford (E)-2-hydroxy-N-methyl-N-(1⁴-methyl-3-oxo-55-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (Compound 35) and (Z)-2-hydroxy-N-methyl-N-(1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (Compound 36). Compounds 37 and 38.

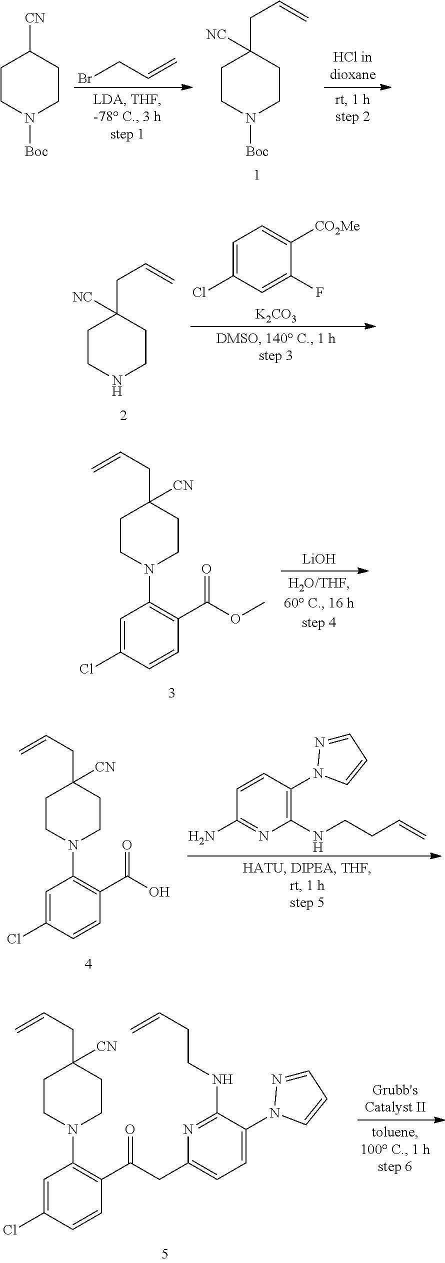

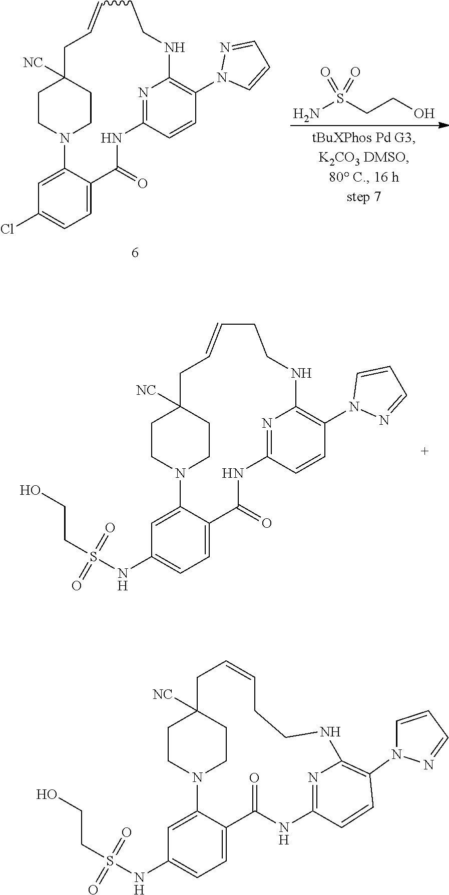 [SMILES: N#CC12CC=CCCNc3nc(ccc3-n3cccn3)NC(=O)c3ccc(Cl)cc3N(CC1)CC2 ; NS(=O)(=O)CCO ; N#CC12C/C=C/CCNc3nc(ccc3-n3cccn3)NC(=O)c3ccc(NS(=O)(=O)CCO)cc3N(CC1)CC2 ; N#CC12C/C=C\CCNc3nc(ccc3-n3cccn3)NC(=O)c3ccc(NS(=O)(=O)CCO)cc3N(CC1)CC2]

Step 1. tert-butyl 4-allyl-4-cyanopiperidine-1-carboxylate: tert-butyl 4-cyanopiperidine-1-carboxylate (5 g, 0.02 mol) in THF (100 mL) was added to the mixture of 2 M LDA in THF (40 mL) in THF (50 mL) under N₂ atmosphere at −78° C. The mixture was stirred at −78° C. for 3 hours. 3-bromoprop-1-ene (3 g, 0.02 mol) was added to the mixture and stirred at −78° C. for 1 hour. The reaction mixture was diluted with water (100 mL) at −78° C. The resulting mixture was extracted with EA (3×10 mL). The combined organic layers were washed with brine (300 mL), dried over anhydrous Na₂SO₄, and concentrated. The residue was purified by silica gel column chromatography, eluted with EA:PE=1:4 to afford tert-butyl 4-allyl-4-cyanopiperidine-1-carboxylate (4 g, 70% yield). LCMS (ESI-MS) m/z=251.2 [M+H]⁺.

Step 2. 4-allylpiperidine-4-carbonitrile: 2M HCl in 1,4-dioxane (8 mL) was added to tert-butyl 4-allyl-4-cyanopiperidine-1-carboxylate (4 g, 0.02 mol) in EA (10 mL). The mixture was stirred at room temperature for 1 hour. The resulting mixture was concentrated under vacuum. The crude material was used for the next step without further purification. LCMS (ESI-MS) m/z=151.2 [M+H]⁺.

Step 3. Methyl 2-(4-allyl-4-cyanopiperidin-1-yl)-4-chlorobenzoate: K₂CO₃ (14 g, 99 mmol) was added to the mixture of 4-allylpiperidine-4-carbonitrile (3.7 g, 25 mmol), methyl 4-chloro-2-fluorobenzoate (4.6 g, 25 mmol) in DMSO (40 mL). The mixture was stirred at 140° C. for 1 hour. The reaction mixture was diluted with water (400 mL) and extracted with EA (3×400 mL). The combined organic layers were washed with brine (1.5 L), dried over anhydrous Na₂SO₄, filtered, and concentrated. The crude residue was purified by silica gel column chromatography, eluted with EA:PE=1:4 to afford methyl 2-(4-allyl-4-cyanopiperidin-1-yl)-4-chlorobenzoate (2 g, 30% yield). LCMS (ESI-MS) m/z=319.1 [M+H]⁺.

Step 4. 2-(4-allyl-4-cyanopiperidin-1-yl)-4-chlorobenzoic acid: LiOH (0.6 g, 0.03 mol) was added to the mixture of methyl 2-(4-allyl-4-cyanopiperidin-1-yl)-4-chlorobenzoate (2 g, 6 mmol) in THF (60 mL) and Water (20 mL). The mixture was stirred at 60° C. for 16 hours. The mixture was acidified to pH 2 with conc. HCl. The resulting mixture was extracted with EA (3×60 mL). The combined organic layers were washed with brine (100 mL), dried over anhydrous Na₂SO₄, filtered, and concentrated to afford 2-(4-allyl-4-cyanopiperidin-1-yl)-4-chlorobenzoic acid (1.6 g, 80% yield). LCMS (ESI-MS) m/z=305.1 [M+H]⁺.

Step 5. 2-(4-allyl-4-cyanopiperidin-1-yl)-N-(6-(but-3-en-1-ylamino)-5-(1H-pyrazol-1-yl)pyridin-2-yl)-4-chlorobenzamide: DIPEA (1.02 g, 7.87 mmol) was added to the mixture of 2-(4-allyl-4-cyanopiperidin-1-yl)-4-chlorobenzoic acid (800 mg, 2.62 mmol), N²-(but-3-en-1-yl)-3-(1H-pyrazol-1-yl)pyridine-2,6-diamine (602 mg, 2.62 mmol) and HATU (1.50 g, 3.94 mmol) in DMF (10 mL). The mixture was stirred at room temperature for 1 hour. The reaction mixture was diluted with water (100 mL) and extracted with EA (3×100 mL). The combined organic layers were washed with brine (300 mL), dried over anhydrous Na₂SO₄, filtered, and concentrated. The residue was purified by silica gel column chromatography, eluted with EA:PE=1:2 to afford 2-(4-allyl-4-cyanopiperidin-1-yl)-N-(6-(but-3-en-1-ylamino)-5-(1H-pyrazol-1-yl)pyridin-2-yl)-4-chlorobenzamide (500 mg, 36.9% yield). LCMS (ESI-MS) m/z=516.2[M+H]⁺.

Step 6. 2⁵-chloro-3-oxo-5⁵-(1H-pyrazol-1-yl)-4,6-diaza-5(2,6)-pyridina-1(1,4)-piperidina-2(1,2)-benzenacycloundecaphan-9-ene-14-carbonitrile: 2-(4-allyl-4-cyanopiperidin-1-yl)-N-(6-(but-3-en-1-ylamino)-5-(1H-pyrazol-1-yl)pyridin-2-yl)-4-chlorobenzamide (500 mg, 0.969 mmol) was added to a stirred mixture of Dichloro(1,3-dimesityl-2-imidazolidinylidene)(2-isopropoxybenzylidene)ruthenium (60.7 mg, 0.097 mmol) in toluene (20 mL). The resulting mixture was stirred for 1 hour at 100° C. The resulting mixture was concentrated under reduced pressure. The residue was purified by silica gel column chromatography, eluted with EA:PE=1:6 to afford (E)-2⁵-chloro-3-oxo-5⁵-(1H-pyrazol-1-yl)-4,6-diaza-5(2,6)-pyridina-1(1,4)-piperidina-2(1,2)-benzenacycloundecaphan-9-ene-1⁴-carbonitrile (300 mg, 63.5% yield). LCMS (ESI-MS) m/z=488.2[M+H]⁺.

Step 7. Compounds 37 and 38: 2⁵-chloro-3-oxo-5⁵-(1H-pyrazol-1-yl)-4,6-diaza-5(2,6)-pyridina-1(1,4)-piperidina-2(1,2)-benzenacycloundecaphan-9-ene-1⁴-carbonitrile (140 mg, 0.287 gmol) was added to a stirred mixture of t-BuXPhos Pd G3 (30.3 mg, 0.028 mmol), 2-hydroxyethane-1-sulfonamide (108 mg, 0.861 mol) and K₂CO₃ (119 mg, 0.861 mmol) in DMSO (2 mL). The resulting mixture was stirred for 16 hour at 80° C. After cooling to room temperature, the reaction mixture was diluted with H₂O (5 mL) and extracted with EA (2×5 mL). The combined organic layers were washed with brine (10 mL), dried over anhydrous Na₂SO₄, filtered and concentrated under vacuum to afford the crude product. The crude product was purified by Prep-Chiral-HPLC with the following conditions: Column: (R, R)-WHELK-01-Kromasil, 2.12*25 cm, 5 μm; Mobile Phase A: Hex(0.5% 2M NH3-MeOH)--HPLC, Mobile Phase B: MeOH:DCM=1:1--HPLC; Flow rate: 20 ML/MIN mL/min; Gradient: isocratic 60; Wave Length: 254/220 nm nm; RT1 (min): 6.734; RT2 (min): 17.83; Sample Solvent: EtOH--HPLC; Injection Volume: 2.0 mL; Number Of Runs: 3 to afford:

First eluting isomer (Compound 37): (E)-N-(1⁴-cyano-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-2-hydroxyethane-1-sulfonamide (36.0 mg, 26.7% yield).

Second eluting isomer (Compound 38): (Z)—N-(1⁴-cyano-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-2-hydroxyethane-1-sulfonamide (3.44 mg, 10% yield).

Compounds 39 and 40 were prepared according to the procedure outlined for Compounds 31 and 32 using ethanesulfonamide in Step 5 to afford (E)-N-(3-oxo-5⁵-(1H-pyrazol-1-yl)-4,6-diaza-5(2,6)-pyrazina-1(1,4)-piperidina-2(1,2)-benzenacycloundecaphan-9-en-25-yl)ethanesulfonamide (Compound 39) and (Z)—N-(3-oxo-5⁵-(1H-pyrazol-1-yl)-4,6-diaza-5(2,6)-pyrazina-1(1,4)-piperidina-2(1,2)-benzenacycloundecaphan-9-en-2⁵-yl)ethanesulfonamide (Compound 40).

Compounds 41 and 42 were prepared following the procedure for Compounds 31 and 32 using 2-(4-allyl-4-methylpiperidin-1-yl)-4-chlorobenzoic acid in Step 3 to afford (E)-2-hydroxy-N-(1⁴-methyl-3-oxo-5⁵-(1H-pyrazol-1-yl)-4,6-diaza-5(2,6)-pyrazina-1(1,4)-piperidina-2(1,2)-benzenacycloundecaphan-9-en-2⁵-yl)ethane-1-sulfonamide (Compound 41) and (Z)-2-hydroxy-N-(1⁴-methyl-3-oxo-5⁵-(1H-pyrazol-1-yl)-4,6-diaza-5(2,6)-pyrazina-1(1,4)-piperidina-2(1,2)-benzenacycloundecaphan-9-en-2⁵-yl)ethane-1-sulfonamide (Compound 42).

### Compounds 43 and 44.

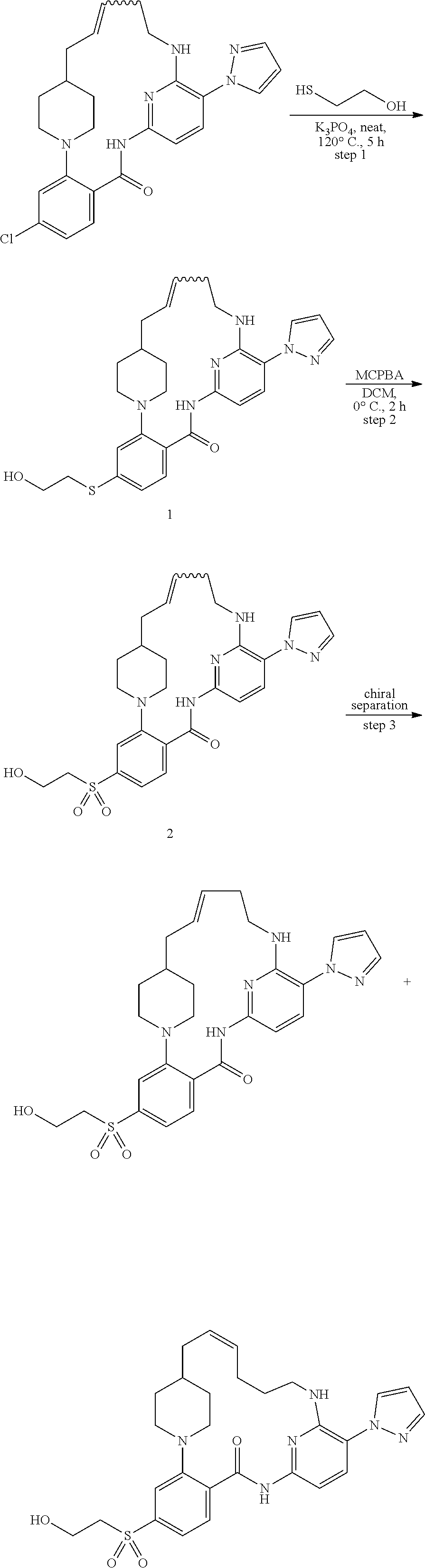

Step 1. 2⁵-((2-hydroxyethyl)thio)-5⁵-(1H-pyrazol-1-yl)-4,6-diaza-5(2,6)-pyridina-1(1,4)-piperidina-2(1,2)-benzenacycloundecaphan-9-en-3-one: 2⁵-chloro-5⁵-(1H-pyrazol-1-yl)-4,6-diaza-5(2,6)-pyridina-1(1,4)-piperidina-2(1,2)-benzenacycloundecaphan-9-en-3-one (500 mg, 1.08 mmol) was added to a stirred mixture of K₃PO₄ (688 mg, 3.24 mmol) in 2-mercaptoethan-1-ol (5 mL). The resulting mixture was stirred for 16 hours at 120° C. After cooling to room temperature, the reaction mixture was diluted with H₂O (5 mL) and extracted with EA (2×5 mL). The combined organic layers were washed with brine (10 mL), dried over anhydrous Na₂SO₄, filtered and concentrated under vacuum to afford the crude product. The residue was purified by silica gel column chromatography, eluted with PE:EA=1:1 to afford 2⁵-((2-hydroxyethyl)thio)-5⁵-(1H-pyrazol-1-yl)-4,6-diaza-5(2,6)-pyridina-1(1,4)-piperidina-2(1,2)-benzenacycloundecaphan-9-en-3-one (300 mg, 55% yield). LCMS (ESI-MS) m/z=505.2 [M+H]⁺.

Step 2. (E)-2⁵-((2-hydroxyethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one: 2⁵-((2-hydroxyethyl)thio)-5⁵-(1H-pyrazol-1-yl)-4,6-diaza-5(2,6)-pyridina-1(1,4)-piperidina-2(1,2)-benzenacycloundecaphan-9-en-3-one (290 mg, 0.575 mmol) was added to a stirred mixture of MCPBA (119 mg, 0.69 mmol) in DCM (5 mL). The resulting mixture was stirred for 2 hours at 0° C. After cooling to room temperature, the reaction mixture was diluted with H₂O (5 mL) and extracted with EA (2×5 mL). The combined organic layers were washed with brine (10 mL), dried over anhydrous Na₂SO₄, filtered and concentrated. The crude residue was purified by silica gel column chromatography, eluted with PE:EA=2:1 to afford 25-((2-hydroxyethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (80 mg, 26% yield). LCMS (ESI-MS) m/z=537.2 [M+H]⁺.

Step 3. Compounds 43 and 44: 2⁵-((2-hydroxyethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (80 mg) was separated by Prep-Chiral-HPLC with the following conditions: Column: XBridge Prep OBD C18 Column, 30*150 mm, 5 μm; Mobile Phase A: Water(10 mmol/L NH4HCO3), Mobile Phase B: ACN; Flow rate: 60 mL/min mL/min; Gradient: 40% B to 60% B in 15 min; Wave Length: 254 nm/220 nm nm; RT1 (min): 9.7 to provide:

First eluting isomer (Compound 43): (E)-2⁵-((2-hydroxyethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (38.5 mg, 54.0% yield). LCMS (ESI-MS) m/z=537.2 [M+H]⁺.

Second eluting isomer (Compound 44): (Z)-2⁵-((2-hydroxyethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (2.2 mg, 22.0% yield). LCMS (ESI-MS) m/z=537.2 [M+H]⁺.

### Compound 45.

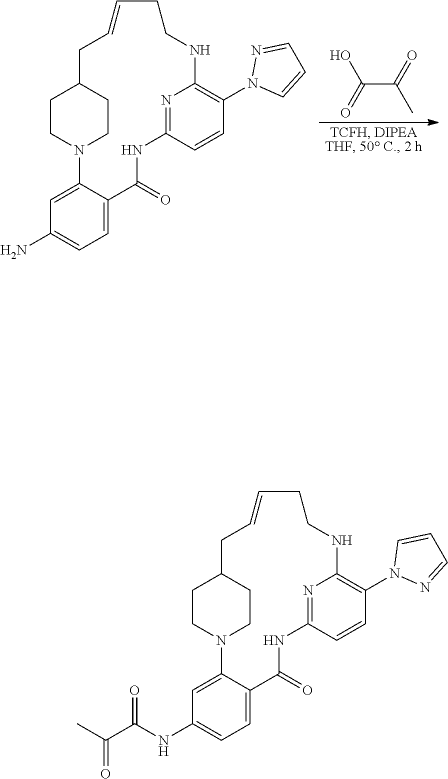

(E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one (50 mg, 0.11 mmol) was added to a stirred mixture of TCFH (47 mg, 0.17 mmol), 2-oxopropanoic acid (30 mg, 0.34 mmol), and DIPEA (44 mg, 0.34 mmol) in THF (2 mL). The resulting mixture was stirred for 2 hours at 50° C. After cooling to room temperature, the reaction mixture was diluted with H₂O (2 mL) and extracted with EA (2×2 mL). The combined organic layers were washed with brine (5 mL), dried over anhydrous Na₂SO₄, filtered and concentrated under vacuum to afford the crude product. The crude product was purified by Prep-HPLC with the following conditions: Column: X Bridge Prep OBD C18 Column, 30*150 mm, 5 μm; Mobile Phase A: Water(H₂O), Mobile Phase B: ACN; Flow rate: 60 mL/min mL/min; Gradient: 55% B to 85% B in 10 min; Wave Length: 254 nm/220 nm nm; RT1 (min): 8.42 afford to (E)-2-oxo-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)propenamide (4.1 mg, 7.0% yield). LCMS (ESI-MS) m/z=514.1 [M+H]⁺.

Compound 46 was prepared according to the procedure outlined for Compound 45 using Intermediate E2 to afford (Z)-2-oxo-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)propenamide.

Compound 47 and 48 were prepared according to the procedure outlined for Compound 29 and 30 using 1,3,4-oxathiazinane 3,3-dioxide and with a reaction temperature of 100° C. in Step 1 to afford (E)-2⁵-(3,3-dioxido-1,3,4-oxathiazinan-4-yl)-1⁴-methyl-55-(1H-pyrazol-1-yl)-4,6-diaza-5(2,6)-pyridina-1(1,4)-piperidina-2(1,2)-benzenacycloundecaphan-9-en-3-one (Compound 47) and (Z)-2⁵-(3,3-dioxido-1,3,4-oxathiazinan-4-yl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (Compound 48).

Compounds 49 and 50 were prepared according to the procedure outlined for Compounds 27 and 28 using 1,3,4-oxathiazinane 3,3-dioxide and increasing the reaction time to 6 hours to afford (E)-2⁵-(3,3-dioxido-1,3,4-oxathiazinan-4-yl)-5⁵-(1H-pyrazol-1-yl)-4,6-diaza-5(2,6)-pyridina-1(1,4)-piperidina-2(1,2)-benzenacycloundecaphan-9-en-3-one (Compound 49) and (Z)-2⁵-(3,3-dioxido-1,3,4-oxathiazinan-4-yl)-5⁵-(1H-pyrazol-1-yl)-4,6-diaza-5(2,6)-pyridina-1(1,4)-piperidina-2(1,2)-benzenacycloundecaphan-9-en-3-one (Compound 50).

Compound 51 was synthesized as described for Intermediate G to afford 1-(25-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid.

### Compounds 52 and 53.

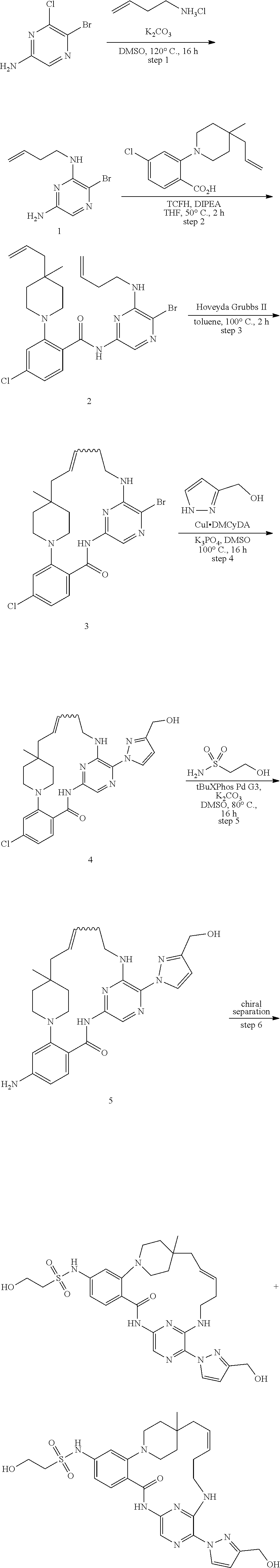

Step 1. 3-bromo-N²-(but-3-en-1-yl)pyrazine-2,6-diamine: 5-bromo-6-chloropyrazin-2-amine (5 g, 20 mmol) was added to a stirred mixture of K₂CO₃ (10 g, 70 mmol) and but-3-en-1-amine, HCl (8 g, 70 mol) in DMSO (100 mL). The resulting mixture was stirred for 16 hours at 120° C. After cooling to room temperature, the reaction mixture was diluted with H₂O (50 mL) and extracted with EA (2×50 mL). The combined organic layers were washed with brine (100 mL), dried over anhydrous Na₂SO₄, filtered and concentrated under vacuum to afford the crude product. The residue was purified by silica gel column chromatography, eluted with PE:EA=9:1 to afford 3-bromo-N²-(but-3-en-1-yl)pyrazine-2,6-diamine (3 g, 50% yield). LCMS (ESI-MS) m/z=244.1 [M+H]⁺.

Step 2. 2-(4-allyl-4-methylpiperidin-1-yl)-N-(5-bromo-6-(but-3-en-1-ylamino)pyrazin-2-yl)-4-chlorobenzamide: 3-bromo-N²-(but-3-en-1-yl)pyrazine-2,6-diamine (1 g, 4 mmol) was added to a stirred mixture of TCFH (2 g, 6 mmol), 2-(4-allyl-4-methylpiperidin-1-yl)-4-chlorobenzoic acid (1 g, 4 mmol) and DIPEA (2 g, 10 mmol) in THF (20 mL). The resulting mixture was stirred for 2 hours at 50° C. After cooling to room temperature, the reaction mixture was diluted with H₂O (10 mL) and extracted with EA (2×10 mL). The combined organic layers were washed with brine (10 mL), dried over anhydrous Na₂SO₄, filtered and concentrated under vacuum to afford the crude product. The residue was purified by silica gel column chromatography, eluted with PE:EA=9:1 to afford 2-(4-allyl-4-methylpiperidin-1-yl)-N-(5-bromo-6-(but-3-en-1-ylamino)pyrazin-2-yl)-4-chlorobenzamide (700 mg, 30% yield). LCMS (ESI-MS) m/z=519.8 [M+H]⁺.

Step 3. 5⁵-bromo-2⁵-chloro-14-methyl-4,6-diaza-5(2,6)-pyrazina-1(1,4)-piperidina-2(1,2)-benzenacycloundecaphan-9-en-3-one: 2-(4-allyl-4-methylpiperidin-1-yl)-N-(5-bromo-6-(but-3-en-1-ylamino)pyrazin-2-yl)-4-chlorobenzamide (680 mg, 1.31 mmol) was added to a stirred mixture of Hoveyda-Grubb's catalyst 2nd Generation (82.1 mg, 13.2 mmol) in toluene (20 mL) and heated to 100° C. After 1 hour, the reaction mixture was cooled to room temperature, concentrated, and purified by silica gel column chromatography, eluted with PE:EA=6:1 to afford 5⁵-bromo-2⁵-chloro-1⁴-methyl-4,6-diaza-5(2,6)-pyrazina-1(1,4)-piperidina-2(1,2)-benzenacycloundecaphan-9-en-3-one (300 mg, 46.6% yield). LCMS (ESI-MS) m/z=491.8 [M+H]⁺.

Step 4. 2⁵-chloro-5⁵-(3-(hydroxymethyl)-1H-pyrazol-1-yl)-1⁴-methyl-4,6-diaza-5(2,6)-pyrazina-1(1,4)-piperidina-2(1,2)-benzenacycloundecaphan-9-en-3-one: 5⁵-bromo-2⁵-chloro-14-methyl-4,6-diaza-5(2,6)-pyrazina-1(1,4)-piperidina-2(1,2)-benzenacycloundecaphan-9-en-3-one (290 mg, 0.591 mmol) was added to a stirred mixture of (1R,2R)—N1,N2-dimethylcyclohexane-1,2-diamine (42.0 mg, 0.295 mmol), (1H-pyrazol-3-yl)methanol (86.9 mg, 0.886 mmol), K₃PO₄ (376 mg, 1.77 mmol) and CuI (56.3 mg, 0.295 mol) in DMSO (5 mL). The resulting mixture was stirred for 16 hour at 100° C. After cooling to room temperature, the reaction mixture was diluted with H₂O (5 mL) and extracted with EA (2×5 mL). The combined organic layers were washed with brine (10 mL), dried over anhydrous Na₂SO₄, filtered and concentrated under vacuum to afford the crude product. The residue was purified by silica gel column chromatography, eluted with PE:EA=3:1 to afford 2⁵-chloro-5⁵-(3-(hydroxymethyl)-1H-pyrazol-1-yl)-1⁴-methyl-4,6-diaza-5(2,6)-pyrazina-1(1,4)-piperidina-2(1,2)-benzenacycloundecaphan-9-en-3-one (100 mg, 33.3% yield). LCMS (ESI-MS) m/z=509.0[M+H]⁺.

Step 5. 2-hydroxy-N-(5⁵-(3-(hydroxymethyl)-1H-pyrazol-1-yl)-1⁴-methyl-3-oxo-4,6-diaza-5(2,6)-pyrazina-1(1,4)-piperidina-2(1,2)-benzenacycloundecaphan-9-en-2⁵-yl)ethane-1-sulfonamide: 2⁵-chloro-5⁵-(3-(hydroxymethyl)-1H-pyrazol-1-yl)-1⁴-methyl-4,6-diaza-5(2,6)-pyrazina-1(1,4)-piperidina-2(1,2)-benzenacycloundecaphan-9-en-3-one (90 mg, 0.18 mmol) was added to a stirred mixture of 2-hydroxyethane-1-sulfonamide (67 mg, 0.53 mmol), K₂CO₃ (73 mg, 0.53 mmol) and t-BuXphos Pd 3 (16 mg, 0.018 mol) in DMSO (1 mL). The resulting mixture was stirred for 80° C. at 16 hours. After cooling to room temperature, the reaction mixture was diluted with H₂O (1 mL) and extracted with EA (2×1 mL). The combined organic layers were washed with brine (2 mL), dried over anhydrous Na₂SO₄, filtered and concentrated under vacuum to afford the crude product. The crude product was purified by Prep-Chiral-HPLC with the following conditions: Column: Xbridge Prep OBD C18 Column, 30*150 mm, 5 μm; Mobile Phase A: Water(10 mmol/L NH4HCO3), Mobile Phase B: ACN; Flow rate: 60 mL/min mL/min; Gradient (B %): 29% B to59% B in 10 min; Wave Length: 254 nm/220 nm nm; RT1 (min): 8.68 to afford 2-hydroxy-N-(5⁵-(3-(hydroxymethyl)-1H-pyrazol-1-yl)-1⁴-methyl-3-oxo-4,6-diaza-5(2,6)-pyrazina-1(1,4)-piperidina-2(1,2)-benzenacycloundecaphan-9-en-2⁵-yl)ethane-1-sulfonamide (50 mg, 47% yield). LCMS (ESI-MS) m/z=597.7[M+H]⁺.

Step 6: Compounds 52 and 53: The previously isolated material was repurified by Prep-Chiral-HPLC with the following conditions: Column: (R, R) Whelk1; Mobile Phase A: Hex: (MeOH:DCM=1:1)=40:60; Flow rate: 1 mL/min mL/min; Gradient: isocratic; Injection Volume: 1 μl mL afford:

First eluting isomer (Compound 52): (E)-2-hydroxy-N-(5⁵-(3-(hydroxymethyl)-1H-pyrazol-1-yl)-1⁴-methyl-3-oxo-4,6-diaza-5(2,6)-pyrazina-1(1,4)-piperidina-2(1,2)-benzenacycloundecaphan-9-en-2⁵-yl)ethane-1-sulfonamide (38.3 mg, 95% yield).

Second eluting isomer (Compound 53): (Z)-2-hydroxy-N-(5⁵-(3-(hydroxymethyl)-1H-pyrazol-1-yl)-1⁴-methyl-3-oxo-4,6-diaza-5(2,6)-pyrazina-1(1,4)-piperidina-2(1,2)-benzenacycloundecaphan-9-en-2⁵-yl)ethane-1-sulfonamide (9.6 mg, 96% yield).

### Compounds 54 and 55.

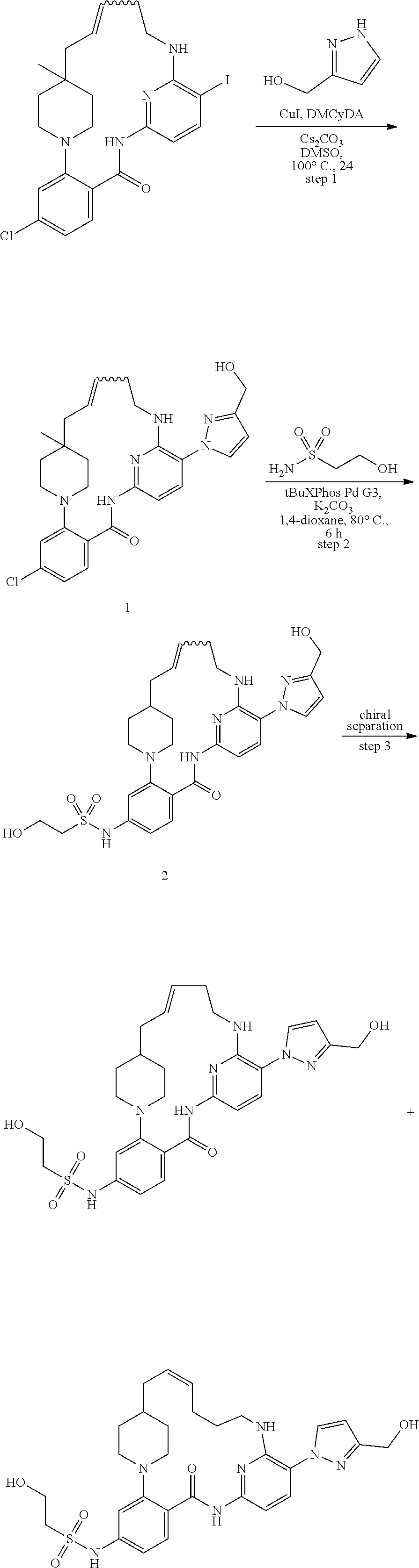

Step 1. 2⁵-chloro-5⁵-(3-(hydroxymethyl)-1H-pyrazol-1-yl)-4,6-diaza-5(2,6)-pyridina-1(1,4)-piperidina-2(1,2)-benzenacycloundecaphan-9-en-3-one: Into a mixture of 2⁵-chloro-5⁵-iodo-4,6-diaza-5(2,6)-pyridina-1(1,4)-piperidina-2(1,2)-benzenacycloundecaphan-9-en-3-one (500 mg, 0.96 mmol) in DMSO (5 mL) was added (1H-pyrazol-3-yl)methanol (188 mg, 1.92 mmol), (1R,2R)—N¹,N²-dimethylcyclohexane-1,2-diamine (273 mg, 1.92 mmol), CuI (365 mg, 1.92 mmol) and Cs₂CO₃ (937 mg, 2.88 mmol) under nitrogen atmosphere. The resulting solution was heated to 100° C. and stirred for 4 hours. After cooling to room temperature, the reaction mixture was diluted with water (200 mL). The resulting mixture was extracted with EA (3×200 mL). The combined organic layers were washed with brine (500 mL), dried over anhydrous Na₂SO₄, and concentrated. The crude residue was purified by silica gel column chromatography, eluted with EA/PE (1:1) to afford 2⁵-chloro-5⁵-(3-(hydroxymethyl)-1H-pyrazol-1-yl)-4,6-diaza-5(2,6)-pyridina-1(1,4)-piperidina-2(1,2)-benzenacycloundecaphan-9-en-3-one (300 mg, 63.7% yield). LCMS (ESI-MS) m/z=493.2 [M+H]⁺.

Step 2. 2-hydroxy-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide: t-BuXphos Pd G3 (48 mg, 61.0 μmol) was added to a mixture of 2⁵-chloro-5⁵-(3-(hydroxymethyl)-1H-pyrazol-1-yl)-4,6-diaza-5(2,6)-pyridina-1(1,4)-piperidina-2(1,2)-benzenacycloundecaphan-9-en-3-one (150 mg, 305 μmol) and 2-hydroxyethane-1-sulfonamide (114 mg, 915 μmol), K₂CO₃ (126 mg, 915 μmol) in DMSO (3 mL) under nitrogen atmosphere. The mixture was stirred at 80° C. for 6 hours. The reaction mixture was diluted with water (150 mL). The resulting mixture was extracted with EA (3×100 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (3:1) to afford 2-hydroxy-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (100 mg, 56.5% yield). LCMS (ESI-MS) m/z=582.2 [M+H]⁺

Step 3. Compounds 54 and 55: 2-hydroxy-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (100 mg) was separated by Prep-Chiral-HPLC with the following conditions (Column: (R, R)-WHELK-01-Kromasil, 2.12*25 cm, 5 μm; Mobile Phase A: Hex--HPLC, Mobile Phase B: EtOH: DCM=1:1--HPLC; Flow rate: 20 ML/MIN mL/min; Gradient (B %): isocratic 50; Wave Length: 254/220 nm nm; RT1 (min): 8.66; RT2 (min): 11.238; Sample Solvent: EtOH--HPLC; Injection Volume: 0.5 mL) to afford:

First eluting isomer (Compound 54): (E)-2-hydroxy-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (17.6 mg, 18% yield).

Second eluting isomer (Compound 55): (Z)-2-hydroxy-N-(5⁵-(3-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (3.4 mg, 3.4% yield). Compounds 56 and 57.

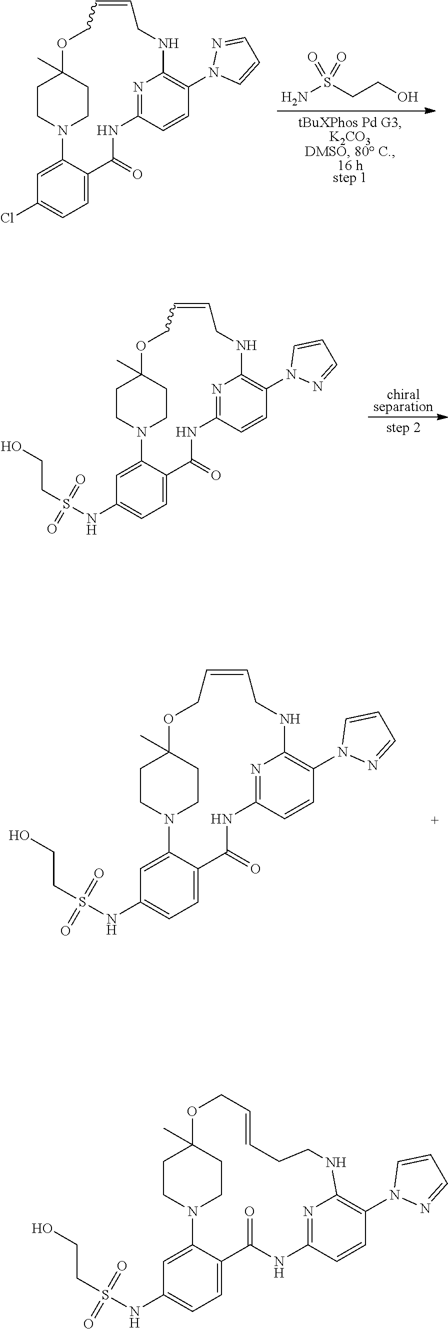

Step 1. 2-hydroxy-N-(14-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethane-1-sulfonamide: 25-chloro-1⁴-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (80 mg, 0.17 mmol) was added to a stirred mixture of t-BuXPhos Pd G3 (18 mg, 0.017 mmol), 2-hydroxyethane-1-sulfonamide (63 mg, 0.50 mmol) and K₂CO₃ (69 mg, 0.50 mmol) in DMSO (2 mL). The resulting mixture was stirred for 16 hour at 80° C. After cooling to room temperature, the reaction mixture was diluted with H₂O (2 mL) and extracted with EA (2×2 mL). The combined organic layers were washed with brine (2 mL), dried over anhydrous Na₂SO₄, filtered and concentrated under vacuum. The crude residue was purified by silica gel column chromatography, eluted with MeOH:DCM=1:9 to afford 2-hydroxy-N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-25-yl)ethane-1-sulfonamide (50 mg, 53% yield). LCMS (ESI-MS) m/z=568.2 [M+H]⁺.

Step 2. Compounds 56 and 57: 2-hydroxy-N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-25-yl)ethane-1-sulfonamide (30 mg) was separated by Prep-HPLC with the following conditions: Column: X select CSH Prep C18 OBD Column, 30*150 mm, 5 μm; Mobile Phase A: Water(0.1% FA), Mobile Phase B: ACN; Flow rate: 60 mL/min mL/min; Gradient: 26% B to 56% B in 10 min; Wave Length: 254 nm/220 nm nm; RT1 (min): 9, 9.67 (min):, 11.1 (min): to afford:

First eluting isomer (Compound 56): (Z)-2-hydroxy-N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethane-1-sulfonamide (5.5 mg, 34% yield).

Second eluting isomer (Compound 57): (E)-2-hydroxy-N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethane-1-sulfonamide (4.4 mg, 29% yield).

### Compounds 58 and 59.

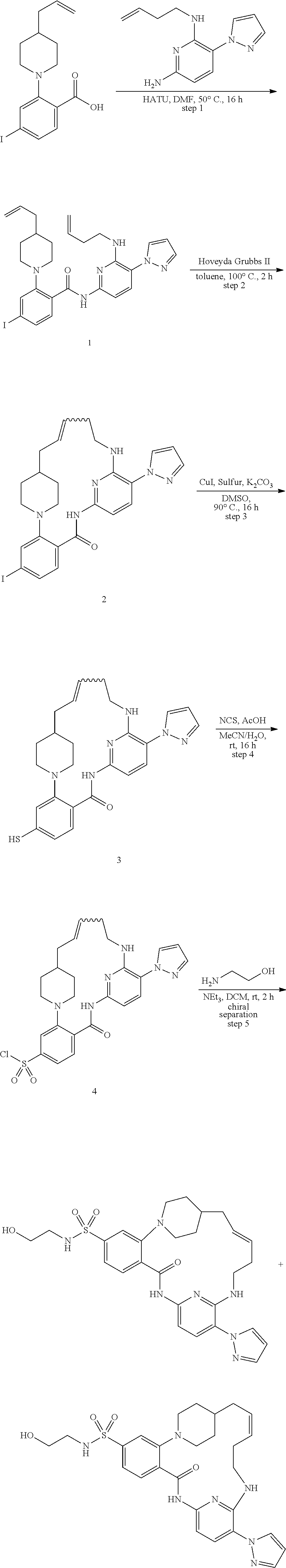

Step 1. 2-(4-allylpiperidin-1-yl)-N-(6-(but-3-en-1-ylamino)-5-(1H-pyrazol-1-yl)pyridin-2-yl)-4-iodobenzamide: 2-(4-allylpiperidin-1-yl)-4-iodobenzoic acid (1 g, 3 mmol) was added to a stirred mixture of HATU (2 g, 4 mmol), N²-(but-3-en-1-yl)-3-(1H-pyrazol-1-yl)pyridine-2,6-diamine (0.7 g, 3 mmol) and DIEA (1 g, 1 mL, 8 mmol) in DMF (10 mL). The resulting mixture was stirred for 16 hours at 50° C. After cooling to room temperature, the reaction mixture was diluted with H₂O (10 mL) and extracted with EA (2×10 mL). The combined organic layers were washed with brine (10 mL), dried over anhydrous Na₂SO₄, filtered and concentrated under vacuum to afford the crude product. The residue was purified by silica gel column chromatography, eluted with PE:EA=6:1 to afford 2-(4-allylpiperidin-1-yl)-N-(6-(but-3-en-1-ylamino)-5-(1H-pyrazol-1-yl)pyridin-2-yl)-4-iodobenzamide (800 mg, 50% yield). LCMS (ESI-MS) m/z=583.4 [M+H]⁺.

Step 2. 2⁵-iodo-5⁵-(1H-pyrazol-1-yl)-4,6-diaza-5(2,6)-pyridina-1(1,4)-piperidina-2(1,2)-benzenacycloundecaphan-9-en-3-one: 2-(4-allylpiperidin-1-yl)-N-(6-(but-3-en-1-ylamino)-5-(1H-pyrazol-1-yl)pyridin-2-yl)-4-iodobenzamide (780 mg, 1.34 mmol) was added to a stirred mixture of dichloro(1,3-dimesityl-2-imidazolidinylidene)(2-isopropoxybenzylidene)ruthenium (83.9 mg, 0.134 mmol) in toluene (20 mL). The resulting mixture was stirred for 1 hour at 100° C. The resulting mixture was concentrated under reduced pressure then purified by silica gel column chromatography, eluted with PE:EA=6:1 to afford 2⁵-iodo-5⁵-(1H-pyrazol-1-yl)-4,6-diaza-5(2,6)-pyridina-1(1,4)-piperidina-2(1,2)-benzenacycloundecaphan-9-en-3-one (400 mg, 53.9% yield). LCMS (ESI-MS) m/z=555.4[M+H]⁺.

Step 3. 2⁵-mercapto-5⁵-(1H-pyrazol-1-yl)-4,6-diaza-5(2,6)-pyridina-1(1,4)-piperidina-2(1,2)-benzenacycloundecaphan-9-en-3-one: Sulfur (347 mg, 1.35 mmol) was added to the mixture of CuI (129 mg, 676 μmol), 2⁵-iodo-5⁵-(1H-pyrazol-1-yl)-4,6-diaza-5(2,6)-pyridina-1(1,4)-piperidina-2(1,2)-benzenacycloundecaphan-9-en-3-one (250 mg, 451 μmol) and K₂CO₃ (187 mg, 1.35 mmol) in DMF (5 mL) nitrogen atmosphere. The mixture was stirred at 90° C. overnight. The reaction mixture was diluted with water (40 mL) and extracted with EA (3×30 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (1:4) to afford 2⁵-mercapto-5⁵-(1H-pyrazol-1-yl)-4,6-diaza-5(2,6)-pyridina-1(1,4)-piperidina-2(1,2)-benzenacycloundecaphan-9-en-3-one (125 mg, 60.2% yield). LCMS (ESI-MS) m/z=461.2[M+H]⁺.

Step 4. 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonyl chloride: AcOH (50 mg, 814 μmol) was added to the mixture of 2⁵-mercapto-5⁵-(1H-pyrazol-1-yl)-4,6-diaza-5(2,6)-pyridina-1(1,4)-piperidina-2(1,2)-benzenacycloundecaphan-9-en-3-one (125 mg, 271 μmol) and NCS (72.5 mg, 543 μmol) in ACN (1 mL) and water (1 mL) under nitrogen atmosphere. The mixture was stirred at room temperature overnight. The reaction mixture was diluted with water (10 mL). The resulting mixture was extracted with EA (3×20 mL). The combined organic layers were washed with brine (100 mL), dried over anhydrous Na₂SO₄. After filtration, the filtrate was concentrated under reduced pressure to afford the product 3-oxo-5⁵-(1H-pyrazol-1-yl)-4,6-diaza-5(2,6)-pyridina-1(1,4)-piperidina-2(1,2)-benzenacycloundecaphan-9-ene-25-sulfonyl chloride (30 mg, 21.0% yield). LCMS (ESI-MS) m/z=527.2[M+H]⁺.

Step 5: Compounds 58 and 59: Triethylamine (5.5 mg, 54 μmol) was added to the mixture of 2-aminoethan-1-ol (3.3 mg, 54 μmol) and N-(2-hydroxyethyl)-3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonamide (30 mg, 54 μmol) in DCM (2 mL). The mixture was stirred at room temperature for 2 hours. The mixture was separated by Prep-Chiral-HPLC with following condition (Column: (R, R) Whelk1; Mobile Phase A: Hex(0.2% FA): (MeOH:DCM=1:1)=40:60; Flow rate: 1 mL/min mL/min; Gradient (B %): isocratic; Injection Volume: 1 μl mL) to afford:

First eluting isomer (Compound 58): (E)-N-(2-hydroxyethyl)-3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonamide (6.3 mg, 20% yield).

Second eluting isomer (Compound 59): (Z)—N-(2-hydroxyethyl)-3-oxo-55-(1H-pyrazol-1-yl)-4,6-diaza-5(2,6)-pyridina-1(1,4)-piperidina-2(1,2)-benzenacycloundecaphan-9-ene-25-sulfonamide (1.4 mg, 4.5% yield).

### Compounds 60 and 61.

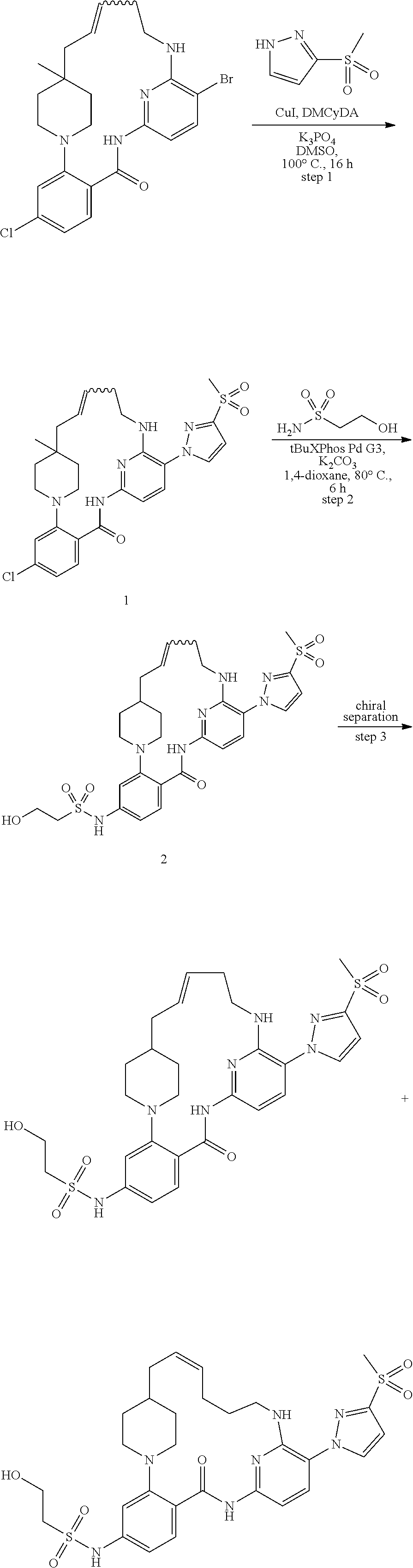

Step 1. 2⁵-chloro-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one: Into a solution of (E)-5⁵-bromo-2⁵-chloro-4,6-diaza-5(2,6)-pyridina-1(1,4)-piperidina-2(1,2)-benzenacycloundecaphan-9-en-3-one (200 mg, 420 μmol) in DMSO (5 mL) was added 3-(methylsulfonyl)-1H-pyrazole (184 mg, 1.26 mmol), (1R,2R)—N¹,N²-dimethylcyclohexane-1,2-diamine (179 mg, 1.26 mmol), CuI (240 mg, 1.26 mmol) and K₃PO4 (268 mg, 1.26 mmol). The resulting mixture was heated to 100° C. and stirred for 16 hours. After cooling to room temperature, the reaction mixture was diluted with water (5 mL), extracted with EA (3×5 mL). The combined organic layers were washed brine (2×5 mL), dried with anhydrous Na₂SO₄, filtered and evaporated to give a crude. The crude was purified with silica gel column, eluted with EA:PE=1:2 to afford the desired product (E)-2⁵-chloro-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (110 mg, 48.4% yield). LCMS (ESI-MS) m/z=541.20 [M+H]⁺.

Step 2. 2-hydroxy-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide: t-BuXphos Pd G3 (29.9 mg, 37.0 μmol) was added to a mixture of (E)-2⁵-chloro-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (100 mg, 185 μmol), 2-hydroxyethane-1-sulfonamide (139 mg, 1.11 mmol), K₂CO₃ (153 mg, 1.11 mmol) in DMSO (10 mL) under nitrogen atmosphere. The mixture was stirred at 100° C. for 16 hours. After cooling to room temperature, the reaction mixture was diluted with water (10 mL) and extracted with EA (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with EA:PE=1:1 to afford (E)-2-hydroxy-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (50 mg, 43% yield). LCMS (ESI-MS) m/z=630.30 [M+H]⁺.

Step 3. Compounds 60 and 61: 2-hydroxy-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (50 mg) was separated by Prep-Chiral HPLC with the following conditions (Column: (R, R)-WHELK-01-Kromasil, 2.12*25 cm, 5 μm; Mobile Phase A: Hex(0.5% 2M NH₃-MeOH)--HPLC, Mobile Phase B: MeOH:DCM=1:1--HPLC; Flow rate: 20 mL/min; Gradient (B %): isocratic 70; Wave Length: 254/220 nm; RT1 (min): 6.403; RT2 (min): 12.664; Sample Solvent: EtOH--HPLC; Injection Volume: 0.8 mL) to afford:

First eluting isomer (Compound 60): (E)-2-hydroxy-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (21.6 mg, 43% yield).

Second eluting isomer (Compound 61): (Z)-2-hydroxy-N-(5⁵-(3-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (3.1 mg, 6.2% yield).

### Compound 62.

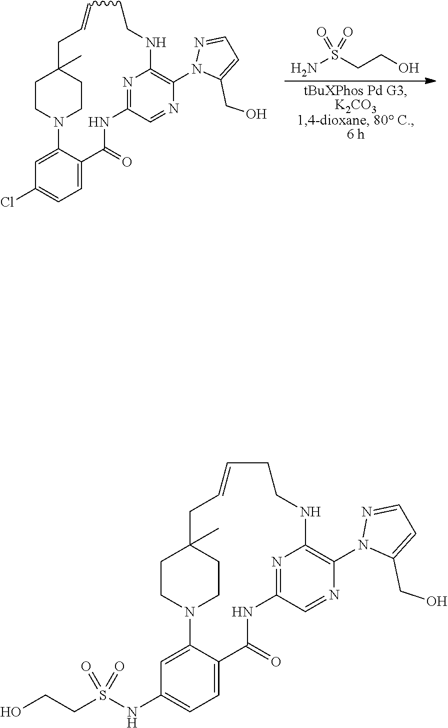

To a solution of 2⁵-chloro-5⁵-(5-(hydroxymethyl)-1H-pyrazol-1-yl)-14-methyl-4,6-diaza-5(2,6)-pyrazina-1(1,4)-piperidina-2(1,2)-benzenacycloundecaphan-9-en-3-one (40 mg, 0.079 mmol, isolated as a byproduct in Step 4 of Compound 52) in DMSO (2 mL) was added t-BuXPhos Pd G3 (17 mg, 0.016 mmol) and K₂CO₃ (33 mg, 0.24 mmol). The resulting mixture was stirred for 16 hours at 80° C. After cooling to room temperature, the reaction mixture was diluted with water (10 mL) and extracted with EA (2×10 mL). The combined organic layers were washed with brine (10 mL), dried with anhydrous Na₂SO₄, filtered and concentrated under vacuum. The residue was purified with silica gel column, eluted with PE:EA=1:1 followed by Chiral-Prep-HPLC with the following conditions: Column: (R, R); Mobile Phase A: Hex: (EtOH:DCM=1:1)=50:50; Flow rate: 1 mL/min mL/min; Gradient (B %): isocratic; Injection Volume: 1 μl mL to afford (E)-2-hydroxy-N-(5⁵-(5-(hydroxymethyl)-1H-pyrazol-1-yl)-14-methyl-3-oxo-4,6-diaza-5(2,6)-pyrazina-1(1,4)-piperidina-2(1,2)-benzenacycloundecaphan-9-en-2⁵-yl)ethane-1-sulfonamide (4.0 mg, 8.2% yield). LCMS (ESI-MS) m/z=597.2 [M+H]⁺.

Compounds 63 and 64 were prepared according to the procedure outlined for Compounds 60 and 61 using 1,3,4-oxathiazinane 3,3-dioxide in step 2 with DMSO as the reaction solvent to afford (E)-2⁵-(3,3-dioxido-1,3,4-oxathiazinan-4-yl)-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (Compound 63) and (Z)-2⁵-(3,3-dioxido-1,3,4-oxathiazinan-4-yl)-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (Compound 64).

Compound 65 was prepared according to the procedure outlined for Compound 3 using 4-(methylsulfonyl)-1H-pyrazole with a reaction time of 4 hours to afford N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

### Compound 66.

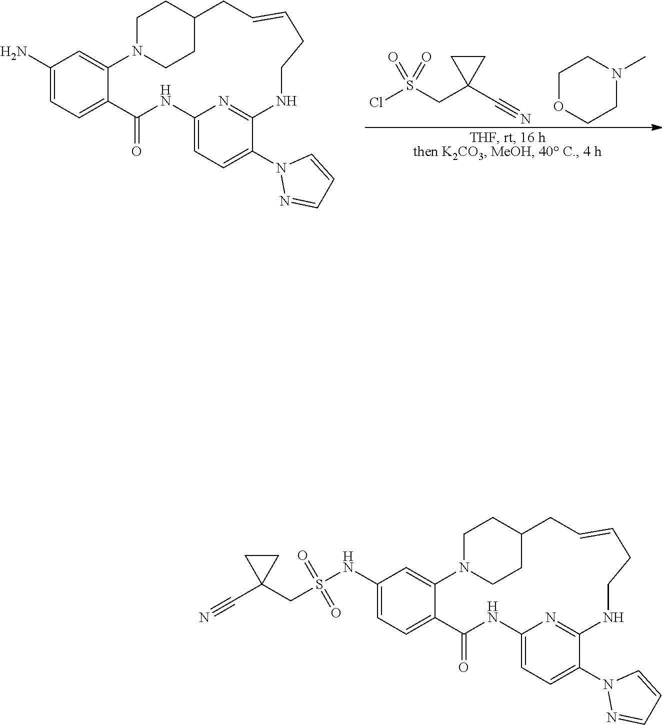

To a vial was added (E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one (17.5 mg, 39.5 μmol), THF (98.6 μL), 4-methylmorpholine (23.9 mg, 26 μL, 237 μmol), and (1-cyanocyclopropyl)methanesulfonyl chloride (21.3 mg, 118 μmol). The reaction mixture was then stirred at rt. After 16 h, K₂CO₃ (5.45 mg, 1 Eq, 39.5 μmol) and MeOH (0.20 mL) were added and the reaction mixture was heated to 40° C. After 4 hour, the reaction mixture was cooled to rt, filtered, and purified via preparative HPLC (10-100% MeCN in H₂O with 0.1% TFA) followed by lyophilization to provide (E)-1-(1-cyanocyclopropyl)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)methanesulfonamide (1.45 mg, 6.26% yield).

Compound 67 was prepared according to the procedure outlined for Compound 66 with ethylsulfamoyl chloride to afford N-(E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one-N′-ethylsulfamide.

Compound 68 was prepared according to the procedure outlined for Compound 66 with 2,2,2-trifluoroethane-1-sulfonyl chloride to afford (E)-2,2,2-trifluoro-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide.

Compound 69 was prepared according to the procedure outlined for Compound 66 with cyanomethanesulfonyl chloride to afford (E)-1-cyano-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)methanesulfonamide.

Compound 70 was prepared according to the procedure outlined for Compound 66 with dimethylsulfamoyl chloride to afford N′-(E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one-N,N-dimethylsulfamide.

### Compound 71.

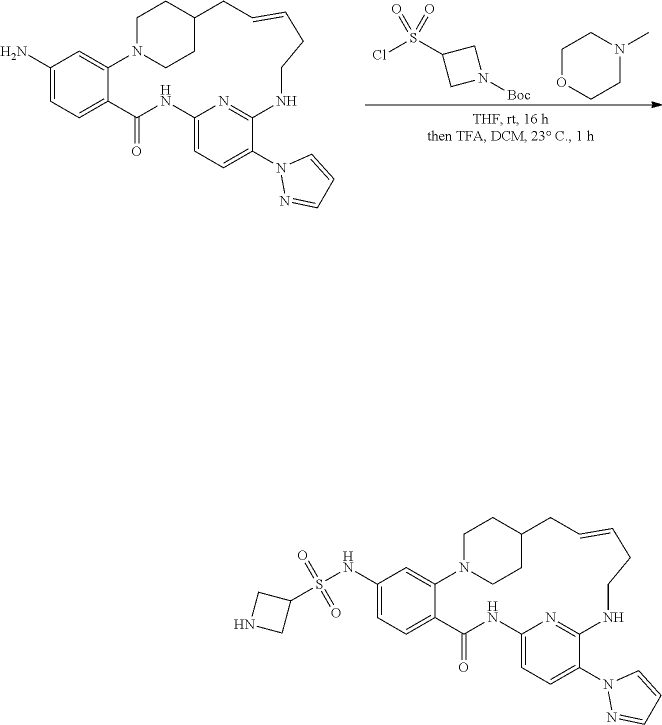

To a vial was added (E)-2⁵-amino-5⁵-(1H-pyrazol-1-yl)-4,6-diaza-5(2,6)-pyridina-1(1,4)-piperidina-2(1,2)-benzenacycloundecaphan-9-en-3-one (17.5 mg, 39.5 μmol), THF (0.20 mL), 4-methylmorpholine (23.9 mg, 26 μL, 237 μmol), and tert-butyl 3-(chlorosulfonyl)azetidine-1-carboxylate (30.3 mg, 118 μmol). The reaction mixture was then stirred at rt. After 16 h, the reaction mixture was concentrated in vacuo then DCM (0.20 mL) and TFA (45.0 mg, 30.4 μL, 395 mol) were added. After 2 hour, the reaction mixture was cooled to rt, concentrated, and purified via preparative HPLC (10-100% MeCN in H₂O with 0.1% TFA) followed by lyophilization to provide (E)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)azetidine-3-sulfonamide 2,2,2-trifluoroacetate (5.1 mg, 23% yield).

Compound 72 was prepared according to the procedure outlined for Compound 66 with ((1R,2S)-2-cyanocyclopropyl)methanesulfonyl chloride to afford 1-((1R,2S)-2-cyanocyclopropyl)-N—((E)-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)methanesulfonamide.

Compound 73 was prepared according to the procedure outlined for Compound 66 with oxetane-3-sulfonyl chloride to afford (E)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)oxetane-3-sulfonamide.

Compound 74 was prepared according to the procedure outlined for Compound 66 with but-3-yne-1-sulfonyl chloride to afford (E)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)but-3-yne-1-sulfonamide.

Compound 75 was prepared according to the procedure outlined for Compound 66 with 2-morpholinoethane-1-sulfonyl chloride hydrochloride to afford (E)-2-morpholino-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide.

Compound 76 was prepared according to the procedure outlined for Compound 66 with (3-fluorotetrahydro-2H-pyran-3-yl)methanesulfonyl chloride to afford 1-(dihydro-2H-pyran-3(4H)-ylidene)-N—((E)-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)methanesulfonamide.

Compound 77 was prepared according to the procedure outlined for Compound 66 with 1H-pyrazole-4-sulfonyl chloride to afford (E)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-1H-pyrazole-4-sulfonamide.

Compound 78 was prepared according to the procedure outlined for Compound 66 with 1-methyl-1H-imidazole-4-sulfonyl chloride to afford (E)-1-methyl-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-1H-imidazole-4-sulfonamide.

Compound 79 was prepared according to the procedure outlined for Compound 66 with 1-(2,2-difluoroethyl)-1H-pyrazole-4-sulfonyl chloride to afford (E)-1-(2,2-difluoroethyl)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-1H-pyrazole-4-sulfonamide.

Compound 80 was prepared according to the procedure outlined for Compound 66 with (methylsulfonyl)methanesulfonyl chloride to afford (E)-1-(methylsulfonyl)-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)methanesulfonamide.

Compound 81 was prepared according to the procedure outlined for Compound 66 with 1-methyl-1H-pyrazole-4-sulfonyl chloride to afford (E)-1-methyl-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)-1H-pyrazole-4-sulfonamide.

### Compound 82.

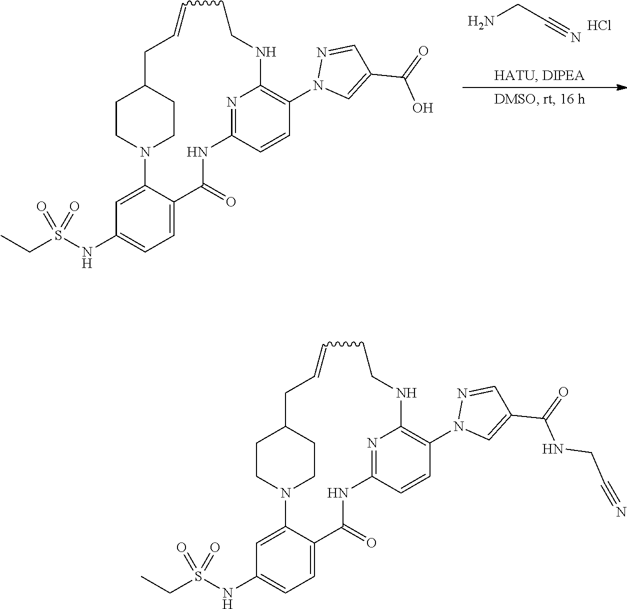

To a vial was added 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid (13.8 mg, 23.8 μmol), 2-aminoacetonitrile hydrochloride (5.51 mg, 59.5 μmol), HATU (13.6 mg, 35.7 μmol), DMSO (0.20 mL) and DIPEA (21.5 mg, 29.0 μL, 167 μmol). The reaction mixture was stirred at rt. After 16 h, the reaction mixture was purified directly via preparative HPLC (10-100% MeCN in water with 0.1% TFA) to provide N-(cyanomethyl)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide (3.61 mg, 24.5% yield).

Compound 83 was prepared according to the procedure outlined for Compound 82 with methylamine hydrochloride to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-methyl-1H-pyrazole-4-carboxamide.

Compound 84 was prepared according to the procedure outlined for Compound 82 with 2-aminoethan-1-ol to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-(2-hydroxyethyl)-1H-pyrazole-4-carboxamide.

Compound 85 was prepared according to the procedure outlined for Compound 82 with morpholine to afford N-(5⁵-(4-(morpholine-4-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 86 was prepared according to the procedure outlined for Compound 82 with 1-(difluoromethyl)cyclopropan-1-amine hydrochloride to afford N-(1-(difluoromethyl)cyclopropyl)-1-(25-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide.

Compound 87 was prepared according to the procedure outlined for Compound 82 with azetidine hydrochloride to afford N-(5⁵-(4-(azetidine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 88 was prepared according to the procedure outlined for Compound 82 with azetidin-3-ol to afford N-(5⁵-(4-(3-hydroxyazetidine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 89 was prepared according to the procedure outlined for Compound 82 with pyrrolidin-3-ol to afford N-(5⁵-(4-(3-hydroxypyrrolidine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 90 was prepared according to the procedure outlined for Compound 82 with 2-aminopropan-1-ol to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-(1-hydroxypropan-2-yl)-1H-pyrazole-4-carboxamide.

Compound 91 was prepared according to the procedure outlined for Compound 82 with dimethylamine hydrochloride to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N,N-dimethyl-1H-pyrazole-4-carboxamide.

Compound 92 was prepared according to the procedure outlined for Compound 82 with 2,2-difluoroethan-1-amine hydrochloride to afford N-(2,2-difluoroethyl)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxamide.

Compound 93 was prepared according to the procedure outlined for Compound 82 with 1-aminopropan-2-ol to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-(2-hydroxypropyl)-1H-pyrazole-4-carboxamide.

Compound 94 was prepared according to the procedure outlined for Compound 82 with (1-aminocyclopropyl)methanol hydrochloride to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-(1-(hydroxymethyl)cyclopropyl)-1H-pyrazole-4-carboxamide.

Compound 95 was prepared according to the procedure outlined for Compound 82 with 3-fluoroazetidine hydrochloride N-(5⁵-(4-(3-fluoroazetidine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 96 was prepared according to the procedure outlined for Compound 82 with 1-methylpiperazine to afford N-(5⁵-(4-(4-methylpiperazine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

Compound 97 was prepared according to the procedure outlined for Compound 82 with 2,2,2-trifluoroethan-1-amine hydrochloride to afford 1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-N-(2,2,2-trifluoroethyl)-1H-pyrazole-4-carboxamide.

Compound 98 was prepared according to the procedure outlined for Compound 82 with 3-(fluoromethyl)azetidin-3-ol hydrochloride to afford N-(5⁵-(4-(3-(fluoromethyl)-3-hydroxyazetidine-1-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

### Compounds 99 and 100.

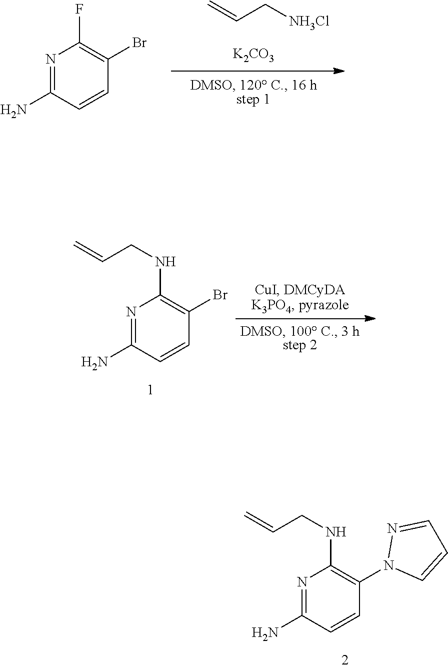

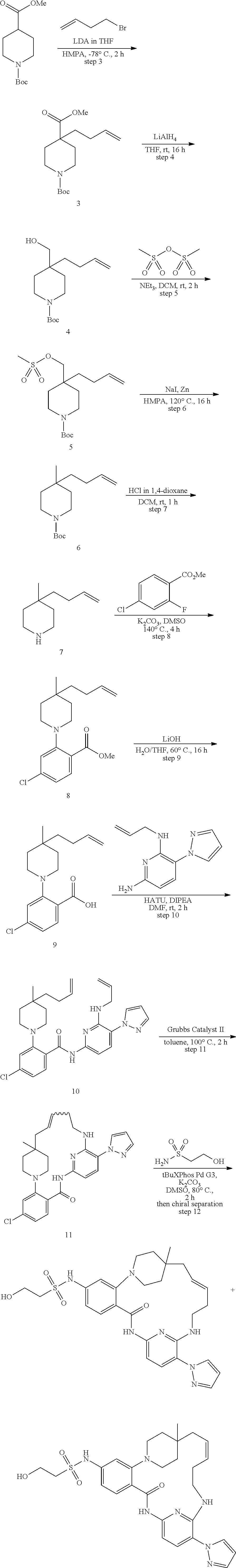

Step 1. N²-allyl-3-bromopyridine-2,6-diamine: K₂CO₃ (10 g, 0.08 mol) was added to a mixture of 5-bromo-6-fluoropyridin-2-amine (5 g, 0.03 mol) and prop-2-en-1-amine (2 g, 0.03 mol) in DMSO (50 mL). The resulting mixture was heated to 120° C. and stirred for 16 hours. After cooling to room temperature, the reaction mixture was diluted with water (500 mL) and extracted with EA (3×200 mL). The combined organic layers were washed with brine (200 mL×2), dried with anhydrous Na₂SO₄, filtered and concentration under vacuum to afford the crude product. The residue was purified with silica gel column chromatography, eluted with EA:PE=1:5 to afford the desired product N²-allyl-3-bromopyridine-2,6-diamine (4.2 g, 70% yield). LCMS (ESI-MS) m/z=227.9 [M+H]⁺.

Step 2. N²-allyl-3-(1H-pyrazol-1-yl) pyridine-2,6-diamine: Into a solution of N²-allyl-3-bromopyridine-2,6-diamine (4.2 g, 18 mmol) in DMSO (42 mL) was added (1R,2R)—N¹, N²-dimethylcyclohexane-1,2-diamine (7.9 g, 55 mmol), 1H-pyrazole (3.8 g, 55 mmol), CuI (7.0 g, 37 mmol) and K₃PO₄ (12 g, 55 mmol). The resulting solution was heated to 100° C. and stirred for 3 hours. After cooling to room temperature, the reaction mixture was filtered and concentrated. The residue was purified with silica gel column chromatography, eluted with EA:PE=1:1 to give the desired product N²-allyl-3-(1H-pyrazol-1-yl) pyridine-2,6-diamine (3.2 g, 81% yield). LCMS (ESI-MS) m/z=216.2 [M+H]⁺.

Step 3. 1-(tert-butyl) 4-methyl 4-(but-3-en-1-yl)piperidine-1,4-dicarboxylate: To a stirred mixture of 1-(tert-butyl) 4-methyl piperidine-1,4-dicarboxylate (5 g, 0.02 mol) in THF (50 mL) was added LDA (4 g, 0.04 mol) dropwise at −78° C. under nitrogen atmosphere. The resulting mixture was stirred at −78° C. for 30 min under nitrogen atmosphere. To the above mixture was added 4-bromobut-1-ene (6 g, 0.04 mol) and HMPA (4 g, 4 mL, 0.02 mol) dropwise at −78° C. The resulting mixture was stirred at room temperature for additional 1.5 h. The reaction was quenched by the addition of sat. NH₄Cl (aq.) (50 mL) at 0° C. The resulting mixture was extracted with EtOAc (3×50 mL). The combined organic layers were washed with brine (2×50 mL), dried over anhydrous Na₂SO₄, filtered and concentrated to afford 1-(tert-butyl) 4-methyl 4-(but-3-en-1-yl)piperidine-1,4-dicarboxylate (5.7 g, 90% yield). LCMS (ESI-MS) m/z=298.2 [M+H]⁺.

Step 4. tert-butyl 4-(but-3-en-1-yl)-4-(hydroxymethyl)piperidine-1-carboxylate: To a stirred mixture of 1-(tert-butyl) 4-methyl 4-(but-3-en-1-yl)piperidine-1,4-dicarboxylate (5.7 g, 19 mmol) in THF (57 mL) was added LiAlH₄ (1.1 g, 29 mmol) at 0° C. under nitrogen atmosphere. The resulting mixture was stirred at room temperature for an additional 16 h. The reaction was quenched by the addition of sat. NH₄Cl (aq.) (100 mL) at 0° C. The resulting mixture was extracted with EtOAc (3×50 mL). The combined organic layers were washed with brine (2×50 mL), dried over anhydrous Na₂SO₄, filtered and concentrated to afford tert-butyl 4-(but-3-en-1-yl)-4-(hydroxymethyl)piperidine-1-carboxylate (3.1 g, 60% yield). LCMS (ESI-MS) m/z=270.2 [M+H]⁺.

Step 5. tert-butyl 4-(but-3-en-1-yl)-4-(((methylsulfonyl)oxy)methyl)piperidine-1-carboxylate: Triethylamine (3.5 g, 4.8 mL, 35 mmol), tert-butyl 4-(but-3-en-1-yl)-4-(hydroxymethyl)piperidine-1-carboxylate (3.1 g, 12 mmol) and methanesulfonic anhydride (4.0 g, 23 mmol) in DCM (31 mL) was stirred at room temperature for 2 hours. The resulting mixture was extracted with EtOAc (3×50 mL). The combined organic layers were washed with brine (2×50 mL), dried over anhydrous Na₂SO₄. After filtration, the filtrate was concentrated under reduced pressure to afford tert-butyl 4-(but-3-en-1-yl)-4-(((methylsulfonyl)oxy)methyl)piperidine-1-carboxylate (1.8 g, 40% yield). LCMS (ESI-MS) m/z=348.2 [M+H]⁺.

Step 6. tert-butyl 4-(but-3-en-1-yl)-4-methylpiperidine-1-carboxylate: A mixture of tert-butyl 4-(but-3-en-1-yl)-4-(((methylsulfonyl)oxy)methyl)piperidine-1-carboxylate (1.8 g, 5.2 mmol), Zinc (1.7 g, 26 mmol), Sodium iodide (2.1 g, 14 mmol) in HMPA (20 mL) was stirred at 120° C. for 16 h. The reaction was quenched by the addition of sat. NH₄Cl (aq.) (100 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure to afford tert-butyl 4-(but-3-en-1-yl)-4-methylpiperidine-1-carboxylate (1.3 g, 99% yield). LCMS (ESI-MS) m/z=254.3 [M+H]⁺.

Step 7. 4-(but-3-en-1-yl)-4-methylpiperidine: HCl in 1,4-dioxane (4 M, 13 mL) was added to stirred mixture of tert-butyl 4-(but-3-en-1-yl)-4-methylpiperidine-1-carboxylate (1.3 g, 5.1 mmol) in DCM (13 mL). The resulting mixture was stirred at room temperature for 1 hour and concentrated under vacuum to afford the crude product 4-(but-3-en-1-yl)-4-methylpiperidine (750 mg, 92% yield). LCMS (ESI-MS) m/z=154.3 [M+H]⁺.

Step 8. Methyl 2-(4-(but-3-en-1-yl)-4-methylpiperidin-1-yl)-4-chlorobenzoate: K₂CO₃ (2.03 g, 14.7 mmol) was added to a stirred mixture of 4-(but-3-en-1-yl)-4-methylpiperidine (750 mg, 4.89 mmol) and methyl 4-chloro-2-fluorobenzoate (969 mg, 5.14 mmol) in DMSO (8 mL). The resulting solution was stirred at 140° C. for 3 hours, quenched by addition of water (100 mL) and extracted with EA (3×50 mL). The combined organic layers were washed with brine (100 mL), dried with anhydrous Na₂SO₄, filtered and concentrated under vacuum. The residue was purified with silica gel column, eluted with EA:PE=1:1 to give the desired product methyl 2-(4-(but-3-en-1-yl)-4-methylpiperidin-1-yl)-4-chlorobenzoate (1 g, 60% yield). LCMS (ESI-MS) m/z=322.2 [M+H]⁺.

Step 9. 2-(4-(but-3-en-1-yl)-4-methylpiperidin-1-yl)-4-chlorobenzoic acid: LiOH (0.4 g, 0.02 mol) in water (5 mL) was added to a mixture of methyl 2-(4-(but-3-en-1-yl)-4-methylpiperidin-1-yl)-4-chlorobenzoate (1 g, 3 mmol) in THF (10 mL). The resulting mixture was heated to 60° C. and stirred for 16 hours. After cooling to room temperature. The mixture was acidified to pH 2 with 1 M. HCl. The resulting mixture was extracted with EtOAc (3×100 mL). The combined organic layers were washed with brine (2×100 mL), dried over anhydrous Na₂SO₄. After filtration, the filtrate was concentrated under reduced pressure to afford 2-(4-(but-3-en-1-yl)-4-methylpiperidin-1-yl)-4-chlorobenzoic acid (680 mg, 70% yield). LCMS (ESI-MS) m/z=308.2 [M+H]⁺.

Step 10. N-(6-(allylamino)-5-(1H-pyrazol-1-yl)pyridin-2-yl)-2-(4-(but-3-en-1-yl)-4-methylpiperidin-1-yl)-4-chlorobenzamide: DIPEA (857 mg, 1.15 mL, 6.63 mmol) was added to a stirred mixture of 2-(4-(but-3-en-1-yl)-4-methylpiperidin-1-yl)-4-chlorobenzoic acid (680 mg, 2.21 mmol), N²-allyl-3-(1H-pyrazol-1-yl)pyridine-2,6-diamine (713 mg, 3.31 mmol) and HATU (1.26 g, 3.31 mmol) in DMF (7 mL). The resulting solution was stirred at room temperature for 3 hours, quenched by addition of water (100 mL) and extracted with EA (3×50 mL). The combined organic layers were washed with brine (50 mL), dried with anhydrous Na₂SO₄, filtered and concentrated under vacuum. The residue was purified with silica gel column, eluted with EA:PE=1:1 to provide N-(6-(allylamino)-5-(1H-pyrazol-1-yl)pyridin-2-yl)-2-(4-(but-3-en-1-yl)-4-methylpiperidin-1-yl)-4-chlorobenzamide (350 mg, 31.% yield). LCMS (ESI-MS) m/z=505.2 [M+H]⁺.

Step 11. 2⁵-chloro-14-methyl-5⁵-(1H-pyrazol-1-yl)-4,6-diaza-5(2,6)-pyridina-1(1,4)-piperidina-2(1,2)-benzenacycloundecaphan-8-en-3-one: Benzylidene(dichloro)(1,3-dimesityl-2-imidazolidinylidene)ruthenium-tricyclohexylphosphine (1:1) (118 mg, 139 μmol) was added to a stirred mixture of N-(6-(allylamino)-5-(1H-pyrazol-1-yl)pyridin-2-yl)-2-(4-(but-3-en-1-yl)-4-methylpiperidin-1-yl)-4-chlorobenzamide (350 mg, 693 μmol) in toluene (4 mL). The resulting solution was stirred at 100° C. for 2 hours, quenched by addition of water (50 mL) and extracted with EA (3×30 mL). The combined organic layers were washed with brine (30 mL), dried with anhydrous Na₂SO₄, filtered and concentrated under vacuum. The residue was purified with silica gel column, eluted with EA:PE=1:1 to give the desired product (Z)-2⁵-chloro-14-methyl-5⁵-(1H-pyrazol-1-yl)-4,6-diaza-5(2,6)-pyridina-1(1,4)-piperidina-2(1,2)-benzenacycloundecaphan-8-en-3-one (50 mg, 15% yield). LCMS (ESI-MS) m/z=477.3 [M+H]⁺.

Step 12. Compounds 99 and 100: A mixture of (Z)-2⁵-chloro-14-methyl-5⁵-(1H-pyrazol-1-yl)-4,6-diaza-5(2,6)-pyridina-1(1,4)-piperidina-2(1,2)-benzenacycloundecaphan-8-en-3-one (50 mg, 0.10 mmol), 2-hydroxyethane-1-sulfonamide (20 mg, 0.16 mmol), K₂CO₃ (43 mg, 0.31 mmol) and t-BuXPhos Pd G3 (17 mg, 21 μmol) in DMSO (1 mL). The mixture was stirred for 2 hours at 80° C. The reaction was cooled to room temperature. The resulting mixture was extracted with EA (30 mL×3). The combined organic layers were washed with brine (50 mL×2), dried over anhydrous Na₂SO₄. After filtration, the filtrate was concentrated under reduced pressure afford a crude product. The crude product was dissolved in MeOH (2 mL) and purified via Chiral-Preparative HPLC with the following conditions: Column: (R, R)-WHELK-01-Kromasil, 2.12*25 cm, 5 μm; Mobile Phase A: Hex--HPLC, Mobile Phase B: EtOH:DCM=1:1--HPLC; Flow rate: 20 ML/MIN mL/min; Gradient (B %): isocratic 55; Wave Length: 254/220 nm; RT1 (min): 7.892; RT2 (min): 9.813; Sample Solvent: EtOH--HPLC; Injection Volume: 1.6 mL; Number Of Runs: 1 to afford:

First eluting isomer (Compound 99): (Z)-2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-8-en-2⁵-yl)ethane-1-sulfonamide (0.9 mg, 6% yield).

Second eluting isomer (Compound 100): (E)-2-hydroxy-N-(14-methyl-3-oxo-5⁵-(1H-pyrazol-1-yl)-4,6-diaza-5(2,6)-pyridina-1(1,4)-piperidina-2(1,2)-benzenacycloundecaphan-8-en-2⁵-yl)ethane-1-sulfonamide (0.4 mg, 3% yield).

Compounds 101 and 102 were prepared according to the procedure outlined for Compounds 43 and 44 using the material produced from Step 1 of Compound 60 and 61. The procedure for the chiral preparative HPLC purification is as follows: 2⁵-(2-hydroxyethyl)sulfonyl)-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (35 mg) was separated by Prep-chiral-HPLC with the following conditions (Column: (R, R)-WHELK-01-Kromasil, 2.12*25 cm, 5 μm; Mobile Phase A: Hex(0.1% FA)--HPLC, Mobile Phase B: MeOH:DCM=1:1--HPLC; Flow rate: 20 ML/MIN mL/min; Gradient (B %): isocratic 80; Wave Length: 254/220 nm nm; RT1 (min): 5.686; RT2 (min): 10.258; Sample Solvent: EtOH--HPLC; Injection Volume: 0.9 mL; Number Of Runs: 2) to afford:

First eluting isomer (Compound 101): (E)-2⁵-((2-hydroxyethyl)sulfonyl)-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (4.3 mg, 12% yield).

Second eluting isomer (Compound 102): (Z)-2⁵-((2-hydroxyethyl)sulfonyl)-5⁵-(3-(methylsulfonyl)-1H-pyrazol-1-yl)-4,6-diaza-5(2,6)-pyridina-1(1,4)-piperidina-2(1,2)-benzenacycloundecaphan-9-en-3-one (1.2 mg, 3.4% yield).

Compounds 102 and 103 were prepared according to the procedure outlined for Compounds 60 and 61 using Intermediate N in Step 1. The procedure for chiral preparative HPLC purification in step 3 is as follows: 2-hydroxy-N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethane-1-sulfonamide (40 mg) was separated by Prep-Chiral-HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 ML/MIN mL/min; Gradient (B %): isocratic 60; Wave Length: 254/220 nm nm; RT1 (min): 7.505; RT2 (min): 11.56; Sample Solvent: EtOH--HPLC; Injection Volume: 0.6 mL) to afford:

First eluting isomer (Compound 102): (Z)-2-hydroxy-N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethane-1-sulfonamide (19.1 mg, 47.8% yield).

Second eluting isomer (Compound 103): (E)-2-hydroxy-N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethane-1-sulfonamide (1.9 mg, 4.8% yield).

Compounds 105 and 106 were prepared according to the procedure outlined for Compounds 103 and 104 using ethanesulfonamide in Step 2. The procedure for preparative HPLC purification in step 3 is as follows: N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (40 mg) was separated by Prep-HPLC with the following conditions (Column: XBridge Prep OBD C18 Column, 30*150 mm, 5 μm; Mobile Phase A: Water(10 mmol/LNH₄HCO₃), Mobile Phase B: ACN; Flow rate: 60 mL/min mL/min; Gradient (B %): 29% B to59% B in 10 min; Wave Length: 254 nm/220 nm n; RT1 (min): 7.97, 8.43 (min):, 9.27 (min):, 9.70 (min)) to afford:

First eluting isomer (Compound 105): (Z)—N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (5.8 mg, 14% yield).

Second eluting isomer (Compound 106): (E)-N-(1⁴-methyl-5⁵-(3-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (2.6 mg, 6.5% yield).

Compounds 107 and 108 were synthesized following Step 1 of Compounds 60 and 61 using Intermediate N as the starting substrate, followed by Steps 1-3 of Compounds 43 and 44 on the resultant intermediate. The procedure for the final chiral preparative HPLC purification is as follows: 25-((2-hydroxyethyl)sulfonyl)-1⁴-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (30 mg) was separated by Prep-HPLC with the following conditions (Column: Xselect CSH Prep C18 OBD Column, 30*150 mm, 5 μm; Mobile Phase A: Water(0.1% FA), Mobile Phase B: ACN; Flow rate: 60 mL/min mL/min; Gradient (B %): 20% B to 50% B in 10 min; Wave Length: 254 nm/220 nm n; RT1 (min): 9.2, 9.9 (min):) to afford:

First eluting isomer (Compound 107): (Z)-2⁵-((2-hydroxyethyl)sulfonyl)-1⁴-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (7.7 mg, 26% yield).

Second eluting isomer (Compound 108): (E)-2⁵-((2-hydroxyethyl)sulfonyl)-1⁴-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (5.3 mg, 18% yield).

Compounds 109 and 110 were prepared according to the procedure outlined for Compounds 60 and 61 using (1H-pyrazol-4-yl)methanol in step 1. The procedure for chiral preparative HPLC purification is as follows: 2-hydroxy-N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (30 mg) was separated by Prep-Chiral-HPLC with the following conditions (Column: (R, R)-WHELK-01-Kromasil, 2.12*25 cm, 5 μm; Mobile Phase A: Hex(0.1% FA)--HPLC, Mobile Phase B: MeOH:DCM=1:1--HPLC; Flow rate: 20 ML/MIN mL/min; Gradient (B %): isocratic 70; Wave Length: 254/220 nm nm; RT1 (min): 4.411; RT2 (min): 7.769; Sample Solvent: EtOH--HPLC; Injection Volume: 1.1 mL; Number Of Runs: 3) to afford:

First eluting isomer (Compound 109): (E)-2-hydroxy-N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (12.1 mg, 40% yield).

Second eluting isomer (Compound 110): (Z)-2-hydroxy-N-(5⁵-(4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (2.2 mg, 7.3% yield).

Compounds 111 and 112 were prepared according to the procedure outlined for Compounds 60 and 61 using 4-(methylsulfonyl)-1H-pyrazole in Step 1. The procedure for chiral preparative HPLC purification is as follows: 2-hydroxy-N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (35 mg) was separated by Prep-Chiral-HPLC with the following conditions (Column: (R, R)-WHELK-01-Kromasil, 2.12*25 cm, 5 μm; Mobile Phase A: Hex(0.1% FA)--HPLC, Mobile Phase B: MeOH:DCM=1:1--HPLC; Flow rate: 20 ML/MIN mL/min; Gradient (B %): isocratic 80; Wave Length: 254/220 nm nm; RT1 (min): 4.935; RT2 (min): 8.431; Sample Solvent: EtOH--HPLC; Injection Volume: 1.0 mL; Number Of Runs: 2) to afford:

First eluting isomer (Compound 111): (E)-2-hydroxy-N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (11.0 mg, 31% yield).

Second eluting isomer (Compound 112): (Z)-2-hydroxy-N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (0.9 mg, 2.6% yield).

Compounds 113 and 114 were prepared according to the procedure outlined for Compounds 103 and 104 using 1,3,4-oxathiazinane 3,3-dioxide in Step 2. The procedure for chiral preparative HPLC purification in step 3 is as follows: 2⁵-(3,3-dioxido-1,3,4-oxathiazinan-4-yl)-14-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (40 mg) was separated by Prep-HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 ML/MIN mL/min; Gradient (B %): isocratic 60; Wave Length: 254/220 nm nm; RT1 (min): 13.526; RT2 (min): 17.222; Sample Solvent: EtOH--HPLC; Injection Volume: 2.0 mL) to afford:

First eluting isomer (Compound 113): (Z)-2⁵-(3,3-dioxido-1,3,4-oxathiazinan-4-yl)-14-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (7.7 mg, 19% yield).

Second eluting isomer (Compound 114): (E)-2⁵-(3,3-dioxido-1,3,4-oxathiazinan-4-yl)-14-methyl-5⁵-(3-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (4.3 mg, 11% yield).

### Compounds 115, 116, and 117.

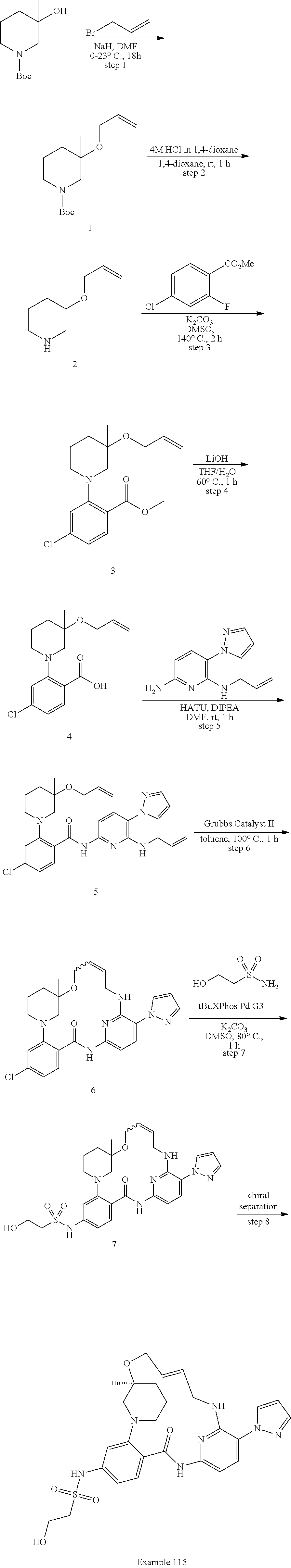

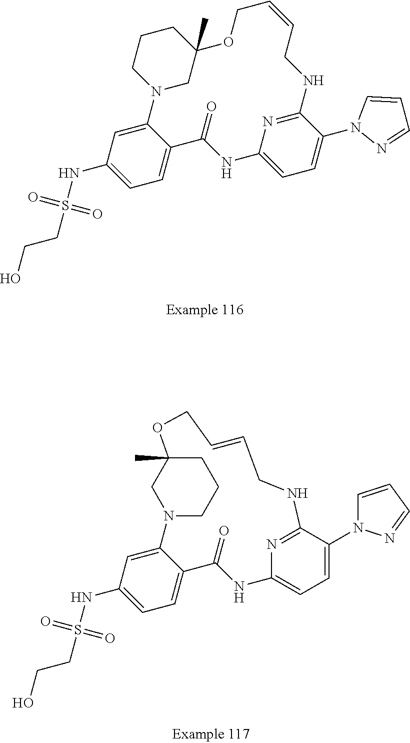 [SMILES: C[C@]12CCCN(C1)c1cc(NS(=O)(=O)CCO)ccc1C(=O)Nc1ccc(-n3cccn3)c(n1)NC/C=C\CO2 ; C[C@@]12CCCN(C1)c1cc(NS(=O)(=O)CCO)ccc1C(=O)Nc1ccc(-n3cccn3)c(n1)NC/C=C/CO2]

Step 1. tert-butyl 3-(allyloxy)-3-methylpiperidine-1-carboxylate: NaH (0.5 g, 0.02 mol) was added to the mixture of tert-butyl 3-hydroxy-3-methylpiperidine-1-carboxylate (3 g, 0.01 mol) and 3-bromoprop-1-ene (2 g, 0.01 mol) in DMF (20 mL). The mixture was stirred at room temperature overnight. The reaction was poured into aq. NH₄Cl (100 mL) at 0° C. The resulting mixture was extracted with EA (3×100 mL). The combined organic layers were washed with brine (300 mL), dried over anhydrous Na₂SO₄. After filtration, the filtrate was concentrated under reduced pressure to afford the crude product tert-butyl 3-(allyloxy)-3-methylpiperidine-1-carboxylate (3.5 g, crude). LCMS (ESI-MS) m/z=256.2 [M+H]⁺.

Step 2. 3-(allyloxy)-3-methylpiperidine: A solution of HCl (4 M in dioxane) was added to the mixture of tert-butyl 3-(allyloxy)-3-methylpiperidine-1-carboxylate (3.5 g, 14 mmol) in 1,4-dioxane (6 mL). The mixture was stirred at room temperature for 1 hour. The reaction mixture was concentrated under vacuum to afford the crude product 3-(allyloxy)-3-methylpiperidine (2.5 g, crude). LCMS (ESI-MS) m/z=156.2 [M+H]⁺.

Step 3. Methyl 2-(3-(allyloxy)-3-methylpiperidin-1-yl)-4-chlorobenzoate: K₂CO₃ (4.0 g, 29 mmol) was added to the mixture of 3-(allyloxy)-3-methylpiperidine (1.5 g, 9.7 mmol) and methyl 4-chloro-2-fluorobenzoate (1.8 g, 9.7 mmol) in DMSO (20 mL). The mixture was stirred at 140° C. for 2 hours. After cooling to room temperature, the reaction was poured into water (200 mL) at room temperature. The resulting mixture was extracted with EA (3×200 mL). The combined organic layers were washed with brine (800 mL), dried over anhydrous Na₂SO₄. After filtration, the filtrate was concentrated under reduced pressure. The residue was purified by silica gel column chromatography, eluted with EA:PE=1:10 to afford methyl 2-(3-(allyloxy)-3-methylpiperidin-1-yl)-4-chlorobenzoate (2.0 g, 64% yield). LCMS (ESI-MS) m/z=324.1 [M+H]⁺.

Step 4. 2-(3-(allyloxy)-3-methylpiperidin-1-yl)-4-chlorobenzoic acid: LiOH (0.9 g, 0.04 mol) was added to the mixture of methyl 2-(3-(allyloxy)-3-methylpiperidin-1-yl)-4-chlorobenzoate (2 g, 6 mmol) in THF (6 mL) and Water (6 mL). The mixture was stirred at 60° C. overnight. After cooling to room temperature, the mixture was acidified to pH 2 with hydrochloric acid solution, the mixture was diluted with H₂O (50 mL) and extracted with EA (2×50 mL). The combined organic layers were washed with brine (50 mL), dried over anhydrous Na₂SO₄, filtered and concentrated under vacuum to afford the crude product 2-(3-(allyloxy)-3-methylpiperidin-1-yl)-4-chlorobenzoic acid (1.8 g, crude). LCMS (ESI-MS) m/z=324.1 [M+H]⁺.

Step 5. N-(6-(allylamino)-5-(1H-pyrazol-1-yl)pyridin-2-yl)-2-(3-(allyloxy)-3-methylpiperidin-1-yl)-4-chlorobenzamide: DIPEA (0.75 g, 5.8 mmol) was added to the mixture of 2-(3-(allyloxy)-3-methylpiperidin-1-yl)-4-chlorobenzoic acid (1.8 g, 5.8 mmol), N²-allyl-3-(1H-pyrazol-1-yl)pyridine-2,6-diamine (1.3 g, 5.8 mmol) and HATU (3.3 g, 8.7 mmol) in DMF (20 mL). The mixture was stirred at room temperature for 1 hour. the reaction was poured into water (200 mL) at room temperature. The resulting mixture was extracted with EA (3×200 mL). The combined organic layers were washed with brine (800 mL), dried over anhydrous Na₂SO₄. After filtration, the filtrate was concentrated under reduced pressure. The residue was purified by silica gel column chromatography, eluted with EA:PE=1:2 to afford N-(6-(allylamino)-5-(1H-pyrazol-1-yl)pyridin-2-yl)-2-(3-(allyloxy)-3-methylpiperidin-1-yl)-4-chlorobenzamide (900 mg, 50% yield). LCMS (ESI-MS) m/z=324.1 [M+H]⁺.

Step 6. 2⁵-chloro-1³-methyl-5⁵-(1H-pyrazol-1-yl)-11-oxa-4,6-diaza-5(2,6)-pyridina-1(1,3)-piperidina-2(1,2)-benzenacycloundecaphan-8-en-3-one: Benzylidene(dichloro)(1,3-dimesityl-2-imidazolidinylidene)ruthenium-tricyclohexylphosphine (1:1) (134 mg, 0.16 mmol) was added to N-(6-(allylamino)-5-(1H-pyrazol-1-yl)pyridin-2-yl)-2-(3-(allyloxy)-3-methylpiperidin-1-yl)-4-chlorobenzamide (800 mg, 1.58 mmol) in toluene (160 mL) under nitrogen atmosphere. The mixture was stirred at 100° C. for 1 hour. The reaction mixture was concentrated under vacuum. The residue was purified by silica gel column chromatography, eluted with EA:PE=1:10 to afford 2⁵-chloro-13-methyl-5⁵-(1H-pyrazol-1-yl)-11-oxa-4,6-diaza-5(2,6)-pyridina-1(1,3)-piperidina-2(1,2)-benzenacycloundecaphan-8-en-3-one (450 mg, 53% yield). LCMS (ESI-MS) m/z=479.2 [M+H]⁺.

Step 7. 2-hydroxy-N-(1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide: t-BuXPhos Pd G3 (145 mg, 93.9 μmol) was added to the mixture of 2⁵-chloro-1³-methyl-5⁵-(1H-pyrazol-1-yl)-11-oxa-4,6-diaza-5(2,6)-pyridina-1(1,3)-piperidina-2(1,2)-benzenacycloundecaphan-8-en-3-one (450 mg, 0.94 μmol), 2-hydroxyethane-1-sulfonamide (353 mg, 2.82 mmol) and K₂CO₃ (390 mg, 2.82 mmol) in DMSO (10 mL) under nitrogen atmosphere. The mixture was stirred at 80° C. for 1 hour. The reaction was poured into water (200 mL) at room temperature. The resulting mixture was extracted with EA (3×100 mL). The combined organic layers were washed with brine (300 mL), dried over anhydrous Na₂SO₄. After filtration, the filtrate was concentrated under reduced pressure. The residue was purified by silica gel column chromatography, eluted with MeOH:DCM (1:10) to afford 2-hydroxy-N-(1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide (300 mg, 56% yield). LCMS (ESI-MS) m/z=568.2 [M+H]⁺.

Step 8. Compounds 115, 116, and 117: 2-hydroxy-N-(1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide (200 mg, 352 μmol) was separated by Prep-Chiral-HPLC with following condition (Column: CHIRAL ART Cellulose-Z; Mobile Phase A: Hex: (EtOH:DCM=1:1)=65:35; Flow rate: 1 mL/min mL/min; Gradient (B %): isocratic; Injection Volume: 1 μl mL) to afford the first eluting isomer, and the remaining mixture was separated by Prep-Chiral HPLC with following conditions Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 ML/MIN mL/min; Gradient (B %): isocratic 40; Wave Length: 254/220 nm nm; RT 1 (min): 10.276; RT 2 (min): 16.706; Sample Solvent: EtOH--HPLC; Injection Volume: 0.6 mL; Number Of Runs: 13 to afford the second and third eluting isomers:

First eluting isomer (Compound 116): stereoisomerically pure 2-hydroxy-N-((1³S,Z)-1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide (3.1 mg, 1.3% yield).

Second eluting isomer (Compound 117): stereoisomerically pure 2-hydroxy-N-((1³S,E)-1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide (29.9 mg, 14.5% yield).

Third eluting isomer (Compound 115): stereoisomerically pure 2-hydroxy-N-((1³R,E)-1³-methyl-3-oxo-5⁵-(1H-pyrazol-1-yl)-11-oxa-4,6-diaza-5(2,6)-pyridina-1(1,3)-piperidina-2(1,2)-benzenacycloundecaphan-8-en-2⁵-yl)ethane-1-sulfonamide (20.1 mg, 10.0% yield).

### Compounds 118 and 119.

N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (20.4 mg) was separated by Prep-Chiral-HPLC with the following conditions (Column: (R, R)-WHELK-01-Kromasil, 2.12*25 cm, 5 μm; Mobile Phase A: Hex(0.1% FA)--HPLC, Mobile Phase B: MeOH:DCM=1:1--HPLC; Flow rate: 20 ML/MIN mL/min; Gradient (B %): isocratic 80; Wave Length: 254/220 nm nn; RT1 (min): 5.264; RT2 (min): 9.523; Sample Solvent: EtOH--HPLC; Injection Volume: 1.8 mL) to afford:

First eluting isomer (Compound 118): (E)-N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (11.2 mg, 55% yield).

Second eluting isomer (Compound 119): (Z)—N-(5⁵-(4-(methylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.5 mg, 12% yield).

### Compounds 120 and 121.

1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid (6.5 mg) was separated by Prep-Chiral-HPLC with the following conditions conditions (Column: (R, R)-WHELK-01-Kromasil, 2.12*25 cm, 5 μm; Mobile Phase A: Hex(0.1% FA)--HPLC, Mobile Phase B: MeOH:DCM=1:1--HPLC; Flow rate: 20 ML/MIN mL/min; Gradient (B %): isocratic 80; Wave Length: 254/220 nm nm; RT1 (min):4.045; RT2 (min): 6.316; Sample Solvent: EtOH--HPLC; Injection Volume: 1.9 mL) to afford:

First eluting isomer (Compound 120): (E)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid (3.6 mg, 55% yield).

Second eluting isomer (Compound 121): (Z)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-4-carboxylic acid (0.5 mg, 8% yield).

### Compound 122.

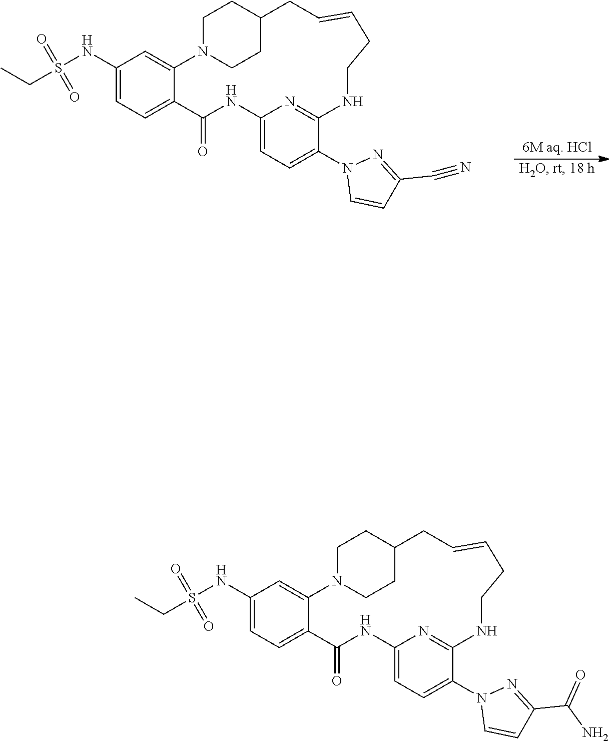 [SMILES: CCS(=O)(=O)Nc1ccc2c(c1)N1CCC(C/C=C/CCNc3nc(ccc3-n3ccc(C#N)n3)NC2=O)CC1 ; "6M aq. HCl" ; "H2O, rt, 18 h" ; CCS(=O)(=O)Nc1ccc2c(c1)N1CCC(C/C=C/CCNc3nc(ccc3-n3ccc(C(N)=O)n3)NC2=O)CC1]

6 M aq. HCl (1 ml) was added to the mixture of (E)-N-(5⁵-(3-cyano-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (10 mg, 18 μmol) in water (1 mL). The mixture was stirred at room temperature overnight. The mixture was purified by Prep-HPLC with the following conditions (UV 254 nm/220 nm Xselect CSH Prep C18 OBD Column, 30*150 mm, 5 μm Water(0.1% FA) ACN 60 mL/min 29% B to 59% B in 8 min 7.5) to afford (E)-1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazole-3-carboxamide (7.1 mg, 69% yield).

### Compound 123.

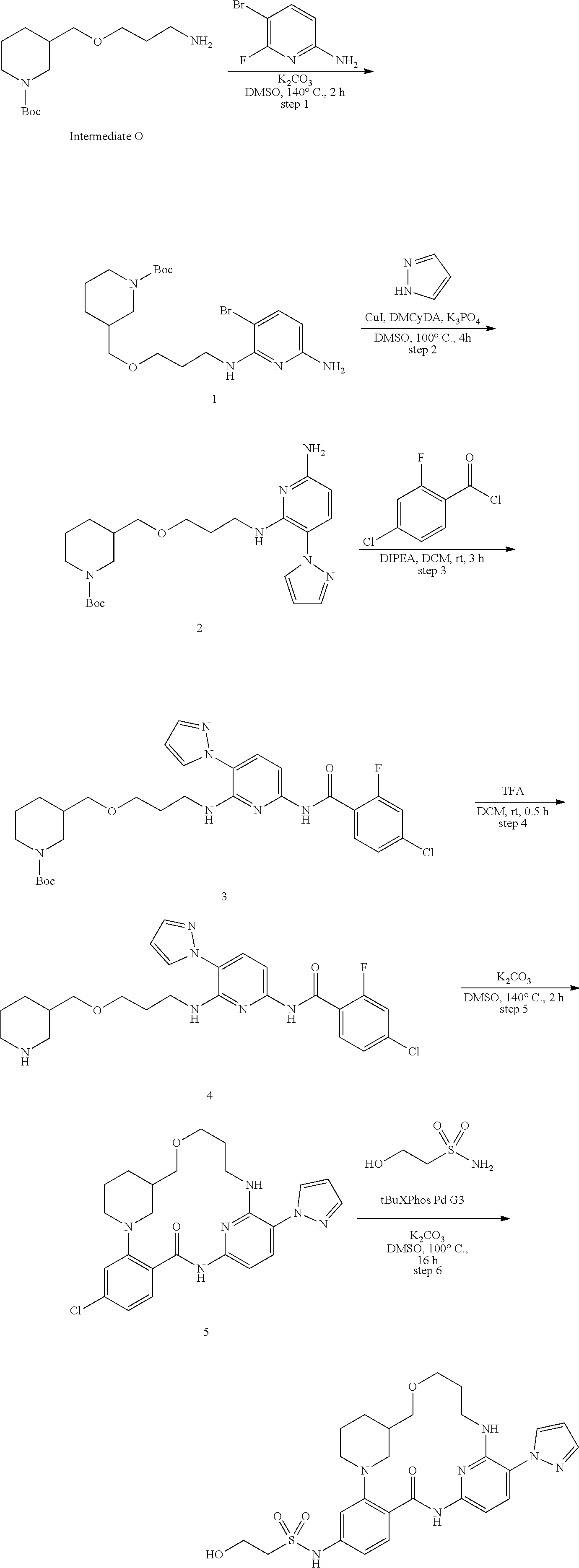

Step 1. tert-butyl 3-((3-((6-amino-3-bromopyridin-2-yl)amino)propoxy)methyl)piperidine-1-carboxylate: K₂CO₃ (6.4 g, 46 mmol) was added to a mixture of tert-butyl 3-((3-aminopropoxy)methyl)piperidine-1-carboxylate, Intermediate O (4.2 g, 15 mmol) and 5-bromo-6-fluoropyridin-2-amine (3.2 g, 17 mmol) in DMSO (40 mL) The resulting mixture was heated to 140° C. and stirred for 2 hours. After cooling to room temperature, the reaction mixture was diluted with water (300 mL) and extracted with EA (3×100 mL). The combined organic layers were washed with brine (3×100 mL), dried with anhydrous Na₂SO₄, filtered and concentrated under vacuum. The residue was purified with silica gel column, eluted with EA/PE 1:1 to afford the desired product tert-butyl 3-((3-((6-amino-3-bromopyridin-2-yl)amino)propoxy)methyl)piperidine-1-carboxylate (2.6 g, 38% yield), LCMS (ESI-MS) m/z=443.1 [M+H]⁺.

Step 2. tert-butyl 3-((3-((6-amino-3-(1H-pyrazol-1-yl) pyridin-2-yl)amino)propoxy)methyl)piperidine-1-carboxylate: Into a solution of tert-butyl 3-((3-((6-amino-3-bromopyridin-2-yl)amino)propoxy)methyl)piperidine-1-carboxylate (1.3 g, 2.9 mmol) in DMSO (26 mL) was added 1H-pyrazole (0.60 g, 8.8 mmol), (1R,2R)—N¹,N²-dimethylcyclohexane-1,2-diamine (1.3 g, 8.8 mmol), CuI (1.1 g, 5.9 mmol) and Potassium phosphate, tribasic (1.9 g, 0.73 mL, 8.8 mmol). The resulting mixture was heated to 100° C. and stirred for 2 hours. After cooling to room temperature, the reaction mixture was filtered and the filtrate was concentrated under vacuum. The residue was purified with silica gel column, eluted with EA/PE 1:3 to afford the desired product tert-butyl 3-((3-((6-amino-3-(1H-pyrazol-1-yl) pyridin-2-yl)amino)propoxy)methyl)piperidine-1-carboxylate (840 mg, 67% yield), LCMS (ESI-MS) m/z=431.3 [M+H]⁺.

Step 3. tert-butyl 3-((3-((6-(4-chloro-2-fluorobenzamido)-3-(1H-pyrazol-1-yl) pyridin-2-yl)amino) propoxy)methyl)piperidine-1-carboxylate: DIPEA (756 mg, 1.02 mL, 5.85 mmol) was added to a mixture of tert-butyl 3-((3-((6-amino-3-(1H-pyrazol-1-yl) pyridin-2-yl)amino)propoxy)methyl)piperidine-1-carboxylate (840 mg, 1.95 mmol) and 4-chloro-2-fluorobenzoyl chloride (452 mg, 2.34 mmol) in DCM (9 mL). The resulting mixture was stirred for 3 hours at rt. The reaction mixture was diluted with water (100 mL) and extracted with EA (3×100 mL). The combined organic layers were washed with brine (100 mL), dried over anhydrous Na₂SO₄, filtered and concentrated under vacuum to afford the crude product. The residue was purified with silica gel column, eluted with EA/PE 1:1 to afford the desired product tert-butyl 3-((3-((6-(4-chloro-2-fluorobenzamido)-3-(1H-pyrazol-1-yl)pyridin-2-yl)amino)propoxy)methyl)piperidine-1-carboxylate (750 mg, 65% yield), LCMS (ESI-MS) m/z=587.3 [M+H]⁺.

Step 4. 4-chloro-2-fluoro-N-(6-((3-(piperidin-3-ylmethoxy)propyl)amino)-5-(1H-pyrazol-1-yl)pyridin-2-yl)benzamide: TFA (7.28 g, 4.92 mL, 63.9 mmol) was added to stirred mixture of tert-butyl 3-((3-((6-(4-chloro-2-fluorobenzamido)-3-(1H-pyrazol-1-yl)pyridin-2-yl)amino)propoxy)methyl)piperidine-1-carboxylate (750 mg, 1.28 mmol) in DCM (5 mL). The resulting mixture was stirred at room temperature for 30 min and concentrated under vacuum to afford the crude product 4-chloro-2-fluoro-N-(6-((3-(piperidin-3-ylmethoxy)propyl)amino)-5-(1H-pyrazol-1-yl)pyridin-2-yl)benzamide (610 mg, crude), LCMS (ESI-MS) m/z=487.2 [M+H]⁺.

Step 5. 2⁵-chloro-5⁵-(1H-pyrazol-1-yl)-10-oxa-4,6-diaza-5(2,6)-pyridina-1(1,3)-piperidina-2(1,2)-benzenacycloundecaphan-3-one: K₂CO₃ (519 mg, 3.76 mmol) was added to a mixture of 4-chloro-2-fluoro-N-(6-((3-(piperidin-3-ylmethoxy)propyl)amino)-5-(1H-pyrazol-1-yl)pyridin-2-yl)benzamide (610 mg, 1.25 mmol) in DMSO (6 mL). The resulting mixture was heated to 140° C. and stirred for 2 hours. After cooling to room temperature, the reaction mixture was diluted with water (100 mL) and extracted with EA (3×50 mL). The combined organic layers were washed with brine (100 mL), dried over anhydrous Na₂SO₄, filtered and concentrated under vacuum to afford desired product 2⁵-chloro-5⁵-(1H-pyrazol-1-yl)-10-oxa-4,6-diaza-5(2,6)-pyridina-1(1,3)-piperidina-2(1,2)-benzenacycloundecaphan-3-one (480 mg, crude). LCMS (ESI-MS) m/z=467.2 [M+H]⁺.

Step 6. Compound 123: A mixture of 2⁵-chloro-5⁵-(1H-pyrazol-1-yl)-10-oxa-4,6-diaza-5(2,6)-pyridina-1(1,3)-piperidina-2(1,2)-benzenacycloundecaphan-3-one (430 mg, 921 μmol), 2-hydroxyethane-1-sulfonamide (138 mg, 1.10 mmol), K₂CO₃ (382 mg, 2.76 mmol) and t-BuXPhos Pd G3 (149 mg, 184 μmol) in DMSO (5 mL) was stirred for 1 hour at 80° C. The resulting mixture was extracted with EA (20 mL×3). The combined organic layers were washed with brine (40 mL×2), dried over anhydrous Na₂SO₄. After filtration, the filtrate was concentrated under reduced pressure afford a crude product. The crude product was purified by preparative TLC (10% MeOH in DCM) to afford 2-hydroxy-N-(3-oxo-5⁵-(1H-pyrazol-1-yl)-10-oxa-4,6-diaza-5(2,6)-pyridina-1(1,3)-piperidina-2(1,2)-benzenacycloundecaphane-2⁵-yl)ethane-1-sulfonamide (82 mg), LCMS (ESI-MS) m/z=556.1 [M+H]⁺.

Compounds 124 and 125 were prepared via chiral separation of Compound 123 using the following conditions: Column: Mobile Phase A: Hex: (EtOH:DCM=1:1)=60:40; Flow rate: 1 mL/min mL/min; Gradient: isocratic; Injection Volume: 1 μl/mL to afford the two products:

First eluting isomer (Compound 124): enantiopure 2-hydroxy-N-(3-oxo-5⁴-(1H-pyrazol-1-yl)-10-oxa-4,6-diaza-1(1,3)-piperidina-2(1,2),5(1,3)-dibenzenacycloundecaphane-2⁵-yl)ethane-1-sulfonamide (34.7 mg, 43% yield).

Second eluting isomer (Compound 125): enantiopure 2-hydroxy-N-(3-oxo-5⁴-(1H-pyrazol-1-yl)-10-oxa-4,6-diaza-1(1,3)-piperidina-2(1,2),5(1,3)-dibenzenacycloundecaphane-2⁵-yl)ethane-1-sulfonamide (30.3 mg, 37% yield).

Compound 126 was prepared from Intermediate P according to the procedure outlined for Compound 123 to afford 2-hydroxy-N-(3-oxo-5⁵-(1H-pyrazol-1-yl)-9-oxa-4,6-diaza-5(2,6)-pyridina-1(1,3)-piperidina-2(1,2)-benzenacycloundecaphane-2⁵-yl)ethane-1-sulfonamide.

### Compounds 126 and 127.

2-hydroxy-N-(3-oxo-5⁵-(1H-pyrazol-1-yl)-9-oxa-4,6-diaza-5(2,6)-pyridina-1(1,3)-piperidina-2(1,2)-benzenacycloundecaphane-2⁵-yl)ethane-1-sulfonamide (30 mg, 54 μmol) was dissolved with MeOH and purified by Chiral-Prep-HPLC with the following conditions: Column: CHIRAL ART Cellulose-SJ, 3*25 cm, 5 μm; Mobile Phase A: Hex(0.1% FA)--HPLC, Mobile Phase B: MeOH:DCM=1:1--HPLC; Flow rate: 30 ML/MIN mL/min; Gradient: isocratic 20; Wave Length: 254/220 nm nm; RT1 (min): 33.741; RT2 (min): 37.858; Sample Solvent: EtOH--HPLC; Injection Volume: 0.6 mL; Number Of Runs: 3 to afford the two products:

First eluting isomer (Compound 126): Enantiopure 2-hydroxy-N-(3-oxo-5⁵-(1H-pyrazol-1-yl)-9-oxa-4,6-diaza-5(2,6)-pyridina-1(1,3)-piperidina-2(1,2)-benzenacycloundecaphane-2⁵-yl)ethane-1-sulfonamide (10.0 mg, 33% yield).

Second eluting isomer (Compound 127): Enantiopure 2-hydroxy-N-(3-oxo-5⁵-(1H-pyrazol-1-yl)-9-oxa-4,6-diaza-5(2,6)-pyridina-1(1,3)-piperidina-2(1,2)-benzenacycloundecaphane-2⁵-yl)ethane-1-sulfonamide (10.1 mg, 34% yield).

Compound 129 was prepared according to the procedure outlined for Compound 123 with Intermediate Q to afford 2-hydroxy-N-(14-methyl-3-oxo-5⁵-(1H-pyrazol-1-yl)-10-oxa-4,6-diaza-5(2,6)-pyridina-1(1,4)-piperidina-2(1,2)-benzenacycloundecaphane-2⁵-yl)ethane-1-sulfonamide.

Compound 130 was prepared according to the procedure outlined for Compound 123 with Intermediate R to afford 2-hydroxy-N-(1³-methyl-3-oxo-5⁵-(1H-pyrazol-1-yl)-10-oxa-4,6-diaza-5(2,6)-pyridina-1(1,3)-piperidina-2(1,2)-benzenacycloundecaphane-2⁵-yl)ethane-1-sulfonamide.

Compounds 131 and 132 was prepared via chiral separation of 2-hydroxy-N-(1³-methyl-3-oxo-5⁵-(1H-pyrazol-1-yl)-10-oxa-4,6-diaza-5(2,6)-pyridina-1(1,3)-piperidina-2(1,2)-benzenacycloundecaphane-2⁵-yl)ethane-1-sulfonamide (50 mg, Compound 130) using the following conditions: (Column: CHIRALPAK IM, 2*25 cm, 5 μm; Mobile Phase A: Hex--HPLC, Mobile Phase B: MeOH:DCM=1:1--HPLC; Flow rate: 20 mL/min; Gradient (B %): isocratic 50; Wave Length: 254/220 nm; RT1 (min): 5.27; RT2 (min): 7.609; Sample Solvent: EtOH--HPLC; Injection Volume: 1.0 mL) to afford:

First eluting isomer (Compound 131): enantiopure 2-hydroxy-N-(1³-methyl-3-oxo-5⁵-(1H-pyrazol-1-yl)-10-oxa-4,6-diaza-5(2,6)-pyridina-1(1,3)-piperidina-2(1,2)-benzenacycloundecaphane-2⁵-yl)ethane-1-sulfonamide (10 mg, 20% yield).

Second eluting isomer (Compound 132): enantiopure 2-hydroxy-N-(1³-methyl-3-oxo-5⁵-(1H-pyrazol-1-yl)-10-oxa-4,6-diaza-5(2,6)-pyridina-1(1,3)-piperidina-2(1,2)-benzenacycloundecaphane-2⁵-yl)ethane-1-sulfonamide (14 mg, 28% yield).

Compound 134 was prepared according to the procedure outlined for Compound 66 using Intermediate E2 and hex-5-yne-1-sulfonyl chloride to afford (Z)—N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)hex-5-yne-1-sulfonamide.

Compound 135 was prepared according to the procedure outlined for Compound 66 using Intermediate E2 and 2,2,2-trifluoroethane-1-sulfonyl chloride to afford (Z)-2,2,2-trifluoro-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide.

Compound 136 was prepared according to the procedure outlined for Compound 66 using Intermediate E2 and 2-cyclopropylethane-1-sulfonyl chloride to afford (Z)-2-cyclopropyl-N-(3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide.

### Compounds 137 and 138.

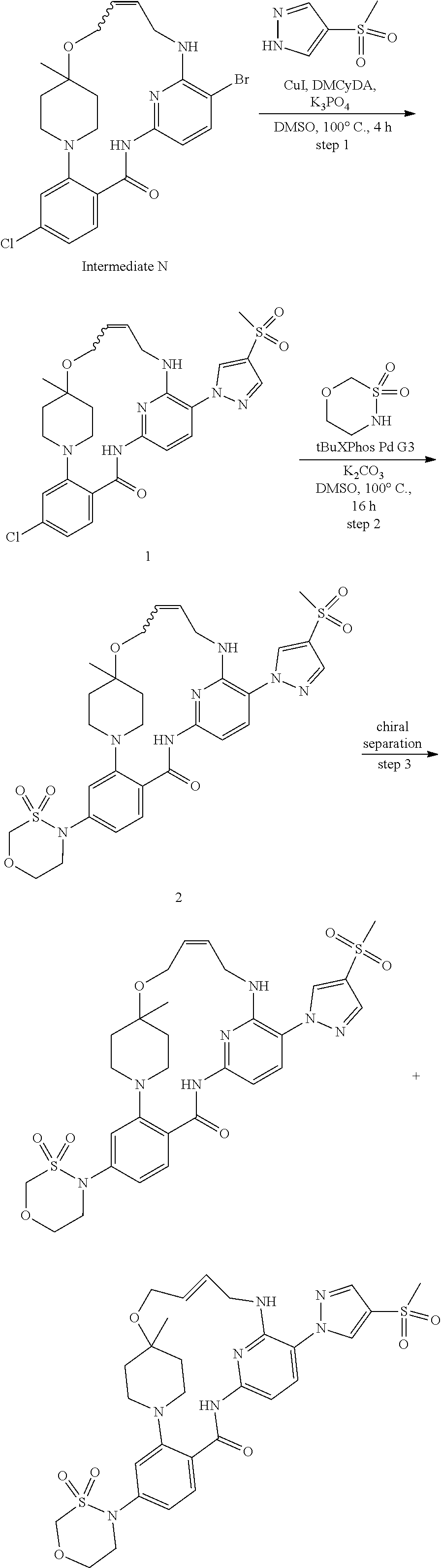

Step 1. 2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one: Into a solution of 5⁵-bromo-2⁵-chloro-1⁴-methyl-11-oxa-4,6-diaza-5(2,6)-pyridina-1(1,4)-piperidina-2(1,2)-benzenacycloundecaphan-8-en-3-one (220 mg, 447 μmol) in DMSO (2 mL) was added 4-(methylsulfonyl)-1H-pyrazole (196 mg, 1.34 mmol), (1R,2R)—N¹,N²-dimethylcyclohexane-1,2-diamine (191 mg, 1.34 mmol), K₃PO₄ (285 mg, 1.34 mmol), CuI (256 mg, 1.34 mmol). The resulting mixture was heated to 100° C. and stirred for 4 hours. After cooling to room temperature, the reaction mixture was diluted with water (2 mL), extracted with EA (3×2 mL). The combined organic layers were washed brine (3×2 mL), dried with anhydrous Na₂SO₄, filtered and evaporated to give a crude. The crude was purified with silica gel column, eluted with EA:PE=1:2 to afford the desired product (Z)-2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (100 mg, 40% yield). LCMS (ESI-MS) m/z=557.2 [M+H]⁺.

Step 2. 2⁵-(3,3-dioxido-1,3,4-oxathiazinan-4-yl)-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one: t-BuXphos Pd G3 (34 mg, 32.0 μmol) was added to a mixture of 2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (90 mg, 0.16 mmol), 2-hydroxyethane-1,3,4-oxathiazinane 3,3-dioxide (66 mg, 0.48 mmol), K₂CO₃ (67 mg, 0.48 mmol) in DMSO (1 mL) under nitrogen atmosphere. The mixture was stirred at 100° C. for 16 hours. After cooling to room temperature, the reaction mixture was diluted with water (1 mL), extracted with EA (3×1 mL). The combined organic layers were washed with brine (2×1 mL), dried over anhydrous Na₂SO₄. After filtration, the filtrate was concentrated under reduced pressure. The residue was purified by Prep-HPLC with the following conditions (Column: XBridge Prep OBD C18 Column, 30*150 mm, 5 μm; Mobile Phase A: Water(10 mmol/LNH4HCO3), Mobile Phase B: ACN; Flow rate: 60 mL/min mL/min; Gradient (B %): 36% B to 66% B in 10 min; Wave Length: 254 nm/220 nm n; RT1 (min): 5.82, 6.28 (min):, 7.23 (min):) to afford (Z)-2⁵-(3,3-dioxido-1,3,4-oxathiazinan-4-yl)-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (40 mg, 38% yield). LCMS (ESI-MS) m/z=658.3 [M+H]⁺.

Step 3. Compounds 137 and 138: 25-(3,3-dioxido-1,3,4-oxathiazinan-4-yl)-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (40 mg, 61 μmol) was dissolved in MeOH (2 mL) and purified with the following conditions (Column: XBridge Prep OBD C18 Column, 30*150 mm, 5 μm; Mobile Phase A: Water(10 mmol/LNH4HCO3), Mobile Phase B: ACN; Flow rate: 60 mL/min mL/min; Gradient (B %): 36% B to 66% B in 10 min; Wave Length: 254 nm/220 nm nm; RT1 (min): 5.82, 6.28 (min):, 7.23 (min):) to afford:

First eluting isomer (Compound 137): (Z)-2⁵-(3,3-dioxido-1,3,4-oxathiazinan-4-yl)-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (5.2 mg, 13% yield).

Second eluting isomer (Compound 138): (E)-2⁵-(3,3-dioxido-1,3,4-oxathiazinan-4-yl)-14-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (2.6 mg, 6.5% yield).

Compounds 139 and 140 were prepared according to the procedure outlined for Compounds 137 and 138 using 1H-pyrazole-3-carbonitrile in Step 1. Purification in Step 3 was provided by Prep-HPLC with following conditions (Column: Xselect CSH Prep Fluoro-Phenyl Column, 30*150 mm, 5 μm; Mobile Phase A: Water(0.1% FA), Mobile Phase B: ACN; Flow rate: 60 mL/min mL/min; Gradient (B %): 26% B to 56% B in 10 min; Wave Length: 254 nm/220 nm n; RT1 (min): 9.8, 10.2 (min) to afford:

First eluting isomer (Compound 139): (Z)-1-(2⁵-(3,3-dioxido-1,3,4-oxathiazinan-4-yl)-14-methyl-3-oxo-1′-oxa-4,6-diaza-5(2,6)-pyridina-1(1,4)-piperidina-2(1,2)-benzenacycloundecaphan-8-en-5⁵-yl)-1H-pyrazole-3-carbonitrile (4.0 mg, 3.3% yield).

Second eluting isomer (Compound 140): (E)-1-(25-(3,3-dioxido-1,3,4-oxathiazinan-4-yl)-14-methyl-3-oxo-1′-oxa-4,6-diaza-5(2,6)-pyridina-1(1,4)-piperidina-2(1,2)-benzenacycloundecaphan-8-en-5⁵-yl)-1H-pyrazole-3-carbonitrile (5.4 mg, 4.5% yield). Compounds 141 and 142.

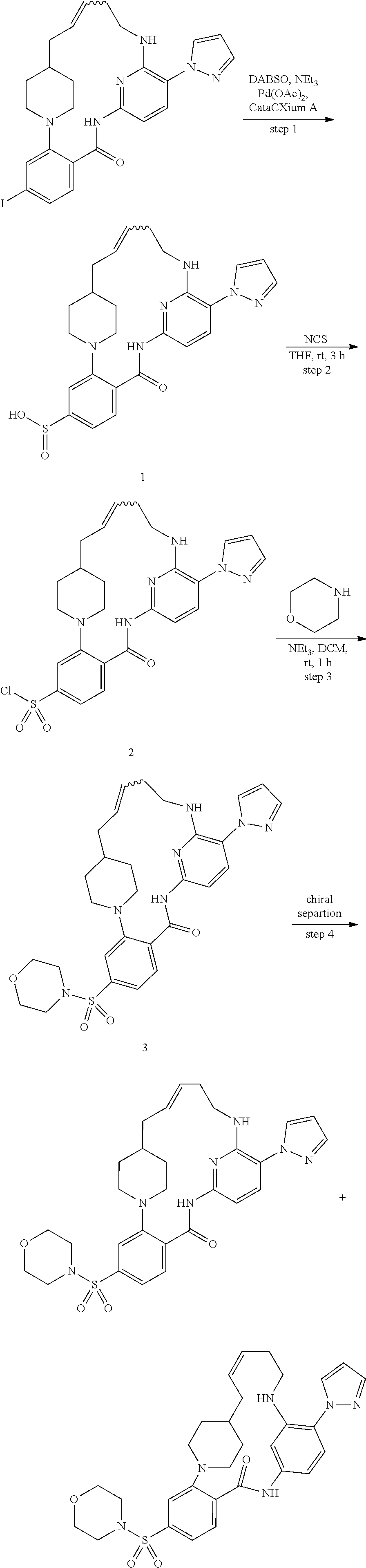

Step 1. 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfinic acid: Into a solution of 2⁵-iodo-5⁵-(1H-pyrazol-1-yl)-4,6-diaza-5(2,6)-pyridina-1(1,4)-piperidina-2(1,2)-benzenacycloundecaphan-9-en-3-one (270 mg, 487 μmol) in 2-propanol (3 mL) was added palladium diacetate (5.47 mg, 24.3 μmol), DABSO (70.2 mg, 292 μmol) and bis(1-adamantyl)-butyl-phosphane (14.0 mg, 39.0 μmol). The resulting solution was heated to 75° C. and stirred for 16 hours. After cooling to room temperature, the reaction mixture was evaporated under vacuum. The crude was purified with silica gel column, eluted with EA:PE=3:2 to afford 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfinic acid (150 mg, 62% yield). LCMS (ESI-MS) m/z=493.2 [M+H]⁺.

Step 2. 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonyl chloride: Into a solution of 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfinic acid (130 mg, 264 μmol) in THF (2 mL) was added NCS (52.9 mg, 396 μmol). The resulting solution was stirred at 25° C. for 3 hours. The reaction mixture was evaporated and purified with silica gel column, eluted with EA:PE=1:1 to afford (E)-3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonyl chloride (90 mg, 65% yield). LCMS (ESI-MS) m/z=527.2 [M+H]⁺.

Step 3. 2⁵-(morpholinosulfonyl)-5⁵-(1H-pyrazol-1-yl)-4,6-diaza-5(2,6)-pyridina-1(1,4)-piperidina-2(1,2)-benzenacycloundecaphan-9-en-3-one: Into a solution of 3-oxo-5⁵-(1H-pyrazol-1-yl)-4,6-diaza-5(2,6)-pyridina-1(1,4)-piperidina-2(1,2)-benzenacycloundecaphan-9-ene-2⁵-sulfonyl chloride (80 mg, 0.15 mmol) in DCM (2 mL) was added triethylamine (46 mg, 63 μL, 0.46 mmol) and morpholine (16 mg, 0.18 mmol). The resulting solution was stirred at 25° C. for 1 hour. The reaction mixture was evaporated and purified with silica gel column, eluted with EA:PE=1:1 to afford (E)-2⁵-(morpholinosulfonyl)-5⁵-(1H-pyrazol-1-yl)-4,6-diaza-5(2,6)-pyridina-1(1,4)-piperidina-2(1,2)-benzenacycloundecaphan-9-en-3-one (50 mg, 57% yield). LCMS (ESI-MS) m/z=578.3 [M+H]⁺.

Step 4. Compounds 141 and 142: 2⁵-(morpholinosulfonyl)-5⁵-(1H-pyrazol-1-yl)-4,6-diaza-5(2,6)-pyridina-1(1,4)-piperidina-2(1,2)-benzenacycloundecaphan-9-en-3-one (50 mg, 1 Eq, 87 μmol) was dissolved in MeOH (1 mL) and purified with the following conditions: Column: (R, R)-WHELK-O1-Kromasil, 2.12*25 cm, 5 μm; Mobile Phase A: Hex(0.5% 2M NH3-MeOH)--HPLC, Mobile Phase B: MeOH:DCM=1:1--HPLC; Flow rate: 20 ML/MIN mL/min; Gradient (B %): isocratic 80; Wave Length: 254/220 nm nm; RT1 (min): 3.931; RT2 (min): 6.851; Sample Solvent: EtOH--HPLC; Injection Volume: 1.2 mL; Number Of Runs: 2. The fractions were collected, evaporated and lyophilized to afford:

First eluting isomer (Compound 141): (E)-2⁵-(morpholinosulfonyl)-5⁵-(1H-pyrazol-1-yl)-4,6-diaza-5(2,6)-pyridina-1(1,4)-piperidina-2(1,2)-benzenacycloundecaphan-9-en-3-one (29.4 mg, 54% yield).

Second eluting isomer (Compound 142): (Z)-2⁵-(morpholinosulfonyl)-5⁵-(1H-pyrazol-1-yl)-4,6-diaza-5(2,6)-pyridina-1(1,4)-piperidina-2(1,2)-benzenacycloundecaphan-9-en-3-one (5.7 mg, 11% yield).

### Compounds 143 and 144.

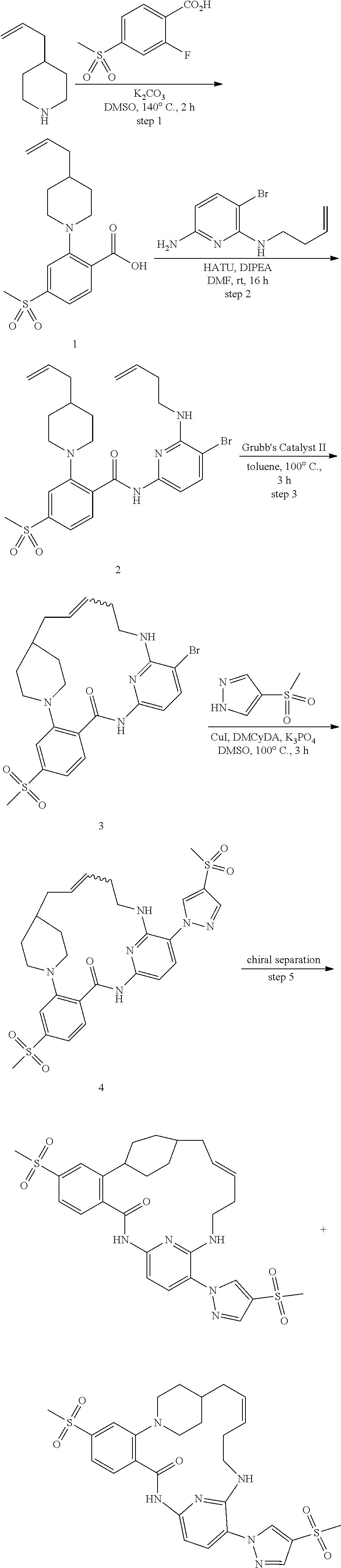

Step 1. 2-(4-allylpiperidin-1-yl)-4-(methylsulfonyl)benzoic acid: K₂CO₃ (1.52 g, 11.0 mmol) was added to a mixture of 4-allylpiperidine (459 mg, 3.67 mmol) and 2-fluoro-4-(methylsulfonyl)benzoic acid (800 mg, 3.67 mmol) in DMSO (8 mL). The resulting mixture was heated to 140° C. and stirred for 2 hours. After cooling to room temperature, the solution was brought to pH=3 and extracted with EA (10 mL). The combined organic layers were washed with brine (10 mL), dried over anhydrous Na₂SO₄, filtered and concentrated under vacuum to afford the crude product. The crude was purified by silica gel column chromatography, eluted with EA:PE=1:1 to afford 2-(4-allylpiperidin-1-yl)-4-(methylsulfonyl)benzoic acid (900 mg, 76% yield). LCMS (ESI-MS) m/z=324.1 [M+H]⁺.

Step 2. 2-(4-allylpiperidin-1-yl)-N-(5-bromo-6-(but-3-en-1-ylamino)pyridin-2-yl)-4-(methylsulfonyl)benzamide: 3-bromo-N²-(but-3-en-1-yl)pyridine-2,6-diamine (659 mg, 2.72 mmol), DIPEA (1.06 g, 8.16 mmol) was added to a stirred mixture of 2-(4-allylpiperidin-1-yl)-4-(methylsulfonyl)benzoic acid (880 mg, 2.72 mmol) and HATU (3.10 g, 8.16 mmol) in DMF (9 mL). The resulting mixture was stirred at 25° C. for 16 hours. The reaction mixture was diluted with water (10 mL) and extracted with EA (3×10 mL). The combined organic layers were washed with brine (10 mL×2), dried with anhydrous Na₂SO₄, filtered and concentrated under vacuum. The residue was purified with silica gel column, eluted with EA:PE=1:5 to afford 2-(4-allylpiperidin-1-yl)-N-(5-bromo-6-(but-3-en-1-ylamino)pyridin-2-yl)-4-(methylsulfonyl)benzamide (790 mg, 53% yield). LCMS (ESI-MS) m/z=549.1 [M+H]⁺.

Step 3. 5⁵-bromo-2⁵-(methylsulfonyl)-4,6-diaza-5(2,6)-pyridina-1(1,4)-piperidina-2(1,2)-benzenacycloundecaphan-9-en-3-one: Into a solution of 2-(4-allylpiperidin-1-yl)-N-(5-bromo-6-(but-3-en-1-ylamino)pyridin-2-yl)-4-(methylsulfonyl)benzamide (750 mg, 1.37 mmol) in toluene (20 mL) was added Benzylidene(dichloro)(1,3-dimesityl-2-imidazolidinylidene)ruthenium-tricyclohexylphosphine (1:1) (233 mg, 274 μmol). The resulting solution was stirred at 100° C. for 3 hours. After cooling to room temperature, the reaction mixture was diluted with water (20 mL), extracted with EA (3×20 mL). The combined organic layers were washed brine (2×20 mL), dried with anhydrous Na₂SO₄, filtered and evaporated to give a crude. The crude was dissolved in DCM (5 mL) and purified with silica gel column (EA:PE=1:5) to afford 5⁵-bromo-2⁵-(methylsulfonyl)-4,6-diaza-5(2,6)-pyridina-1(1,4)-piperidina-2(1,2)-benzenacycloundecaphan-9-en-3-one (400 mg, 56% yield). LCMS (ESI-MS) m/z=521.1 [M+H]⁺.

Step 4. 2⁵-(methylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one: Into a solution of 5⁵-bromo-2⁵-(methylsulfonyl)-4,6-diaza-5(2,6)-pyridina-1(1,4)-piperidina-2(1,2)-benzenacycloundecaphan-9-en-3-one (140 mg, 270 μmol) in DMSO (3 mL) was added 4-(methylsulfonyl)-1H-pyrazole (118 mg, 809 μmol), (1R,2R)—N¹,N²-dimethylcyclohexane-1,2-diamine (115 mg, 809 μmol), CuI (154 mg, 809 μmol) and K₃PO₄ (172 mg, 809 μmol). The resulting mixture was heated to 100° C. and stirred for 3 hours. After cooling to room temperature, the reaction mixture was diluted with water (3 mL), extracted with EA (3×3 mL). The combined organic layers were washed brine (2×3 mL), dried with anhydrous Na₂SO₄, filtered and evaporated to give a crude. The crude was purified with silica gel column, eluted with EA:PE=1:2 to afford 2⁵-(methylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (40 mg, 25% yield). LCMS (ESI-MS) m/z=585.2 [M+H]⁺.

Step 5. Compounds 143 and 144: 2⁵-(methylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (40 mg, 68 μmol) was separated by Prep-Chiral HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 mL/min; Gradient (B %): isocratic 80; Wave Length: 254/220 nm; RT1 (min): 6.54; RT2 (min): 13.418; Sample Solvent: EtOH--HPLC; Injection Volume: 2.1 mL) to afford:

First eluting isomer (Compound 143): (E)-2⁵-(methylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (22.3 mg, 56% yield).

Second eluting isomer (Compound 144): (Z)-2⁵-(methylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (3.4 mg, 8.5% yield).

### Compounds 145 and 146.

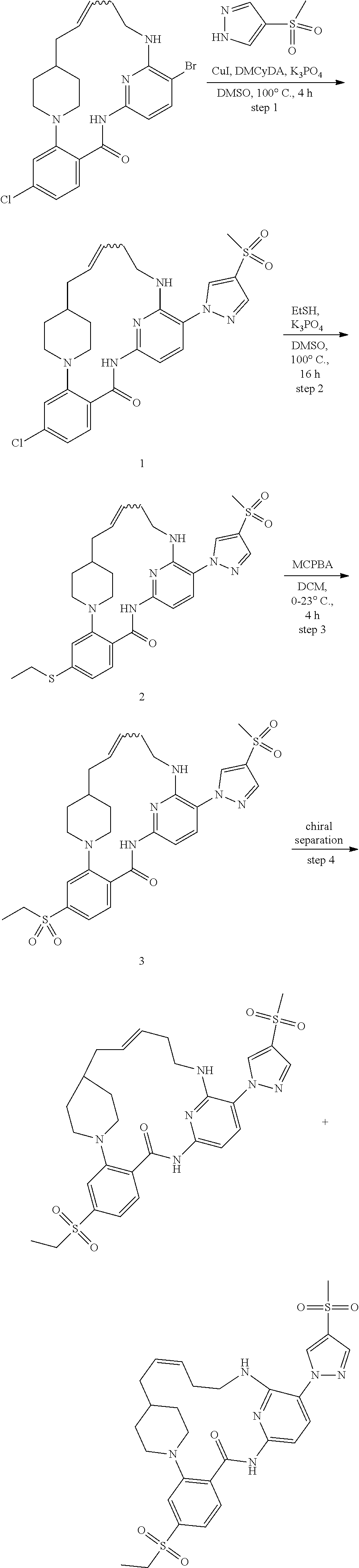

Step 1. 2⁵-chloro-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one: Into a mixture of 5⁵-bromo-2⁵-chloro-4,6-diaza-5(2,6)-pyridina-1(1,4)-piperidina-2(1,2)-benzenacycloundecaphan-9-en-3-one (200 mg, 0.42 mmol) in DMSO (3 mL) was added 4-(methylsulfonyl)-1H-pyrazole (123 mg, 0.84 mmol), (1R,2R)—N¹,N²-dimethylcyclohexane-1,2-diamine (119 mg, 0.84 mmol), CuI (160 mg, 0.84 mmol) and K₃PO₄ (267 mg, 1.26 mmol) under nitrogen atmosphere. The resulting solution was heated to 100° C. and stirred for 4 hours. After cooling to room temperature, the reaction mixture was diluted with water (100 mL). The resulting mixture was extracted with EA (3×100 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:3) to afford 2⁵-chloro-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (110 mg, 48% yield). LCMS (ESI-MS) m/z=541.2 [M+H]⁺.

Step 2. 2⁵-(ethylthio)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one: Into a mixture of 2⁵-chloro-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (100 mg, 0.19 mmol) in DMAc (3 mL) was added ethanethiol (118 mg, 1.90 mmol) and K₂CO₃ (79 mg, 0.57 mmol). The resulting solution was heated to 100° C. and stirred for 16 hours. After cooling to room temperature, The reaction mixture was diluted with water (100 mL). The resulting mixture was extracted with EA (3×100 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:2) to afford 2⁵-(ethylthio)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (100 mg, 92% yield). LCMS (ESI-MS) m/z=567.3 [M+H]⁺.

Step 3. 2⁵-(ethylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one: Into a mixture of 25-(ethylthio)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (90 mg, 0.16 mmol) in DCM (3 mL) was added m-CPBA (55 mg, 0.32 mmol) at 0° C. The resulting solution was stirred for 1 hour at room temperature. Then, the reaction mixture was diluted with saturated sodium bicarbonate (50 mL). The resulting mixture was extracted with EA (3×50 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:1) to afford 2⁵-(ethylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (45 mg, 46% yield). LCMS (ESI-MS) m/z=599.3 [M+H]⁺.

Step 4. Compounds 145 and 146: 2⁵-(ethylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (45 mg) was separated by Prep-chiral-HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 mL/min; Gradient (B %): isocratic 80; Wave Length: 254/220 nm; RT1 (min): 6.705; RT2 (min): 13.541; Sample Solvent: EtOH--HPLC; Injection Volume: 2.2 mL; Number Of Runs: 1) to afford:

First eluting isomer (Compound 145): (E)-2⁵-(ethylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (2.5 mg, 5.6% yield).

second eluting isomer (Compound 146): (Z)-2⁵-(ethylsulfonyl)-5⁵-(4-(methylsulfonyl)-1H-pyrazol-1-yl)-4,6-diaza-5(2,6)-pyridina-1(1,4)-piperidina-2(1,2)-benzenacycloundecaphan-9-en-3-one (1.1 mg, 2.4% yield).

Compounds 147 and 148 were prepared according to the procedure outlined for Compounds 143 and 144 using (1H-pyrazol-4-yl)methanol in Step 4. Purification conditions as follows: 5⁵-(4-(hydroxymethyl)-1H-pyrazol-1-yl)-2⁵-(methylsulfosnyl)-4,6-diaza-5(2,6)-pyridina-1(1,4)-piperidina-2(1,2)-benzenacycloundecaphan-9-en-3-one (40 mg, 75 μmol) was separated by Prep-Chiral HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 mL/min; Gradient (B %): isocratic 80; Wave Length: 254/220 nm; RT1 (min): 5.031; RT2 (min): 9.232; Sample Solvent: EtOH--HPLC; Injection Volume: 1.2 mL) to afford:

First eluting isomer (Compound 147): (E)-5⁵-(4-(hydroxymethyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (16.8 mg, 42% yield).

Second eluting isomer (Compound 148): (Z)-5⁵-(4-(hydroxymethyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (3.2 mg, 8.0% yield).

Compounds 149 and 150 were prepared according to the procedure outlined for Compounds 145 and 146 using (1H-pyrazol-4-yl)methanol in step 1. Purification conditions as follows: 2⁵-(ethylsulfonyl)-5⁵-(4-(hydroxymethyl)-1H-pyrazol-1-yl)-4,6-diaza-5(2,6)-pyridina-1(1,4)-piperidina-2(1,2)-benzenacycloundecaphan-9-en-3-one (40 mg) was separated by Prep-chiral-HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 mL/min; Gradient (B %): isocratic 80; Wave Length: 254/220 nm; RT1 (min): 5.019; RT2 (min): 8.965; Sample Solvent: EtOH--HPLC; Injection Volume: 1.8 mL; Number Of Runs: 1) to afford:

First eluting isomer (Compounds 149): (E)-2⁵-(ethylsulfonyl)-5⁵-(4-(hydroxymethyl)-1H-pyrazol-1-yl)-4,6-diaza-5(2,6)-pyridina-1(1,4)-piperidina-2(1,2)-benzenacycloundecaphan-9-en-3-one (6.8 mg, 17% yield).

Second eluting isomer (Compound 150): (Z)-2⁵-(ethylsulfonyl)-5⁵-(4-(hydroxymethyl)-1H-pyrazol-1-yl)-4,6-diaza-5(2,6)-pyridina-1(1,4)-piperidina-2(1,2)-benzenacycloundecaphan-9-en-3-one (3.0 mg, 7.5% yield).

Compounds 151 and 152 were prepared according to the procedure outlined for Compounds 137 and 138 using 1H-pyrazole-4-carbonitrile in step 1. Purification in step 3 was provided by Prep-HPLC with following conditions: Column: Xselect CSH Prep Fluoro-Phenyl Column, 30*150 mm, 5 μm; Mobile Phase A: Water(0.1% FA), Mobile Phase B: ACN; Flow rate: 60 mL/min mL/min; Gradient (B %): 26% B to 56% B in 10 min; Wave Length: 254 nm/220 nm nm; RT1 (min): 9.8, 10.2 (min) to afford:

First eluting isomer (Compound 151): (Z)-1-(2⁵-(3,3-dioxido-1,3,4-oxathiazinan-4-yl)-14-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-4-carbonitrile (3.6 mg, 3.0% yield).

Second eluting isomer (Compound 152): (E)-1-(25-(3,3-dioxido-1,3,4-oxathiazinan-4-yl)-14-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-4-carbonitrile (3.6 mg, 5.9 μmol, 3.0% yield).

### Compounds 153 and 154.

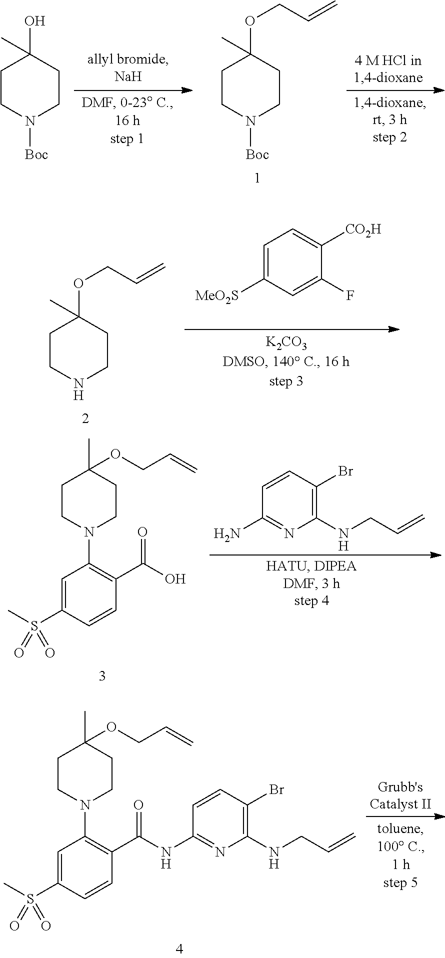

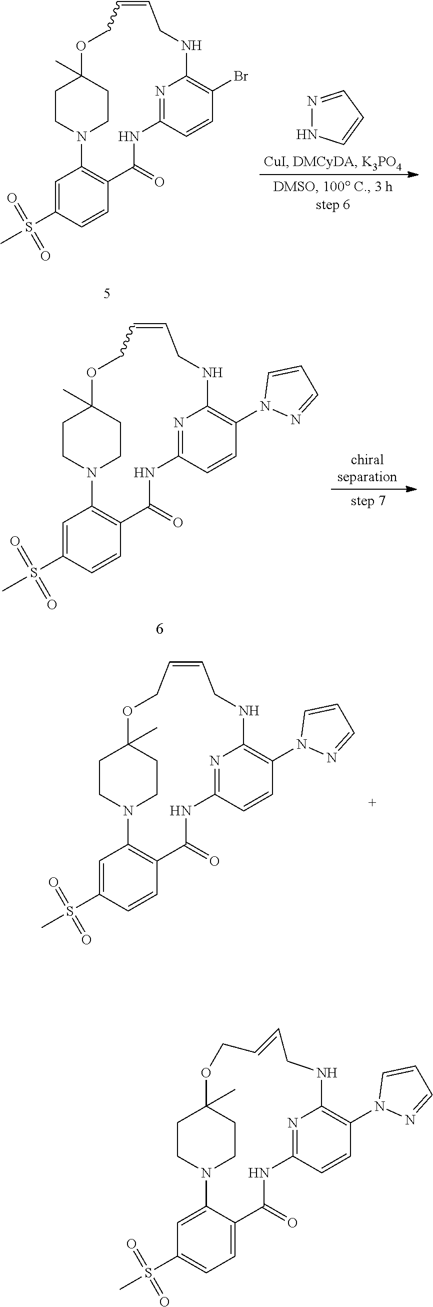 [SMILES: CC12CCN(CC1)c1cc(S(C)(=O)=O)ccc1C(=O)Nc1ccc(Br)c(n1)NCC=CCO2 ; c1cn[nH]c1 ; CC12CCN(CC1)c1cc(S(C)(=O)=O)ccc1C(=O)Nc1ccc(-n3cccn3)c(n1)NCC=CCO2 ; CC12CCN(CC1)c1cc(S(C)(=O)=O)ccc1C(=O)Nc1ccc(-n3cccn3)c(n1)NC/C=C\CO2 ; CC12CCN(CC1)c1cc(S(C)(=O)=O)ccc1C(=O)Nc1ccc(-n3cccn3)c(n1)NC/C=C/CO2]

Step 1. tert-butyl 4-(allyloxy)-4-methylpiperidine-1-carboxylate: NaH (60% in mineral oil, 849 mg, 35.4 mmol) was added to a stirred mixture of tert-butyl 4-hydroxy-4-methylpiperidine-1-carboxylate (5 g, 23.3 mmol) in DMF (50 mL) at 0° C. After stirring for 30 minutes, 3-bromoprop-1-ene (2.80 g, 23.3 mmol) was added. The resulting mixture was stirred at room temperature for 16 hours, quenched by addition of saturated aqueous NH₄Cl (200 mL) and extracted with EA (2×200 mL). The combined organic layers were washed with brine (200 mL), dried with anhydrous Na₂SO₄, filtered and concentrated under vacuum. The residue was purified with silica gel column, eluted with EA:PE=1:1 to afford tert-butyl 4-(allyloxy)-4-methylpiperidine-1-carboxylate (3.4 g, 57% yield). LCMS (ESI-MS) m/z=241.0 [M+H]⁺.

Step 2. 4-(allyloxy)-4-methylpiperidine: 4 M HCl in dioxane (35 ml) was added to tert-butyl 4-(allyloxy)-4-methylpiperidine-1-carboxylate (3.4 g, 13.3 mmol). The resulting mixture was stirred at room temperature for 3 hours and concentrated under vacuum to afford the crude product 4-(allyloxy)-4-methylpiperidine (2.8 g crude). LCMS (ESI-MS) m/z=156.0 [M+H]⁺.

Step 3. 2-(4-(allyloxy)-4-methylpiperidin-1-yl)-4-(methylsulfonyl)benzoic acid: K₂CO₃ (3.65 g, 26.45 mmol) was added to a mixture of 4-(allyloxy)-4-methylpiperidine (820 mg, 5.29 mmol) and 2-fluoro-4-(methylsulfonyl)benzoic acid (961.4 mg, 4.41 mmol) in DMSO (10 mL). The resulting mixture was heated to 140° C. and stirred for 16 hours. After cooling to room temperature, the reaction mixture was diluted with water (50 mL) and extracted with EA (3×50 mL). The combined organic layers were washed with brine (50 mL×2), dried with anhydrous Na₂SO₄, filtered and concentration under vacuum to afford the crude product. The residue was purified with silica gel column, eluted with EA:PE=1:1 to afford 2-(4-(allyloxy)-4-methylpiperidin-1-yl)-4-(methylsulfonyl)benzoic acid (1.1 g, 59% yield). LCMS (ESI-MS) m/z=354.1 [M+H]⁺.

Step 4. N-(6-(allylamino)-5-bromopyridin-2-yl)-2-(4-(allyloxy)-4-methylpiperidin-1-yl)-4-(methylsulfonyl)benzamide: DIPEA (1.207 g, 9.36 mmol) was added to a stirred mixture of 2-(4-(allyloxy)-4-methylpiperidin-1-yl)-4-(methylsulfonyl)benzoic acid (1.1 g, 3.12 mmol), N²-allyl-3-bromopyridine-2,6-diamine (708.2 mg, 3.12 mmol) and HATU (979.7 mg, 6.24 mmol) in DMF (5 mL). The resulting mixture was stirred at room temperature for 3 hours, diluted with water (10 mL), and extracted with EA (3×10 mL). The combined organic layers were washed with brine (10 mL×2), dried with anhydrous Na₂SO₄, filtered and concentrated under vacuum. The residue was purified with silica gel column, eluted with EA:PE=1:1 to afford N-(6-(allylamino)-5-bromopyridin-2-yl)-2-(4-(allyloxy)-4-methylpiperidin-1-yl)-4-(methylsulfonyl)benzamide (1.5 g, 85.6% yield). LCMS (ESI-MS) m/z=563.1 [M+H]⁺.

Step 5. 5⁵-bromo-1⁴-methyl-2⁵-(methylsulfonyl)-11-oxa-4,6-diaza-5(2,6)-pyridina-1(1,4)-piperidina-2(1,2)-benzenacycloundecaphan-8-en-3-one: N-(6-(allylamino)-5-bromopyridin-2-yl)-2-(4-(allyloxy)-4-methylpiperidin-1-yl)-4-(methylsulfonyl)benzamide (1.5 g, 2.67 mmol) was added to a stirred mixture of Benzylidene(1,3-bis(2,4,6-trimethylpheyl-2-imidazolidinylidene)dichloro(tricyclohexylphosphine)ruthenium (453.2 mg, 0.53 mmol) in toluene (15 mL). The resulting mixture was stirred for 1 hour at 100° C. then concentrated under reduced pressure. The residue was purified by silica gel column chromatography, eluted with PE:EA (5:1) to afford 5⁵-bromo-1⁴-methyl-2⁵-(methylsulfonyl)-11-oxa-4,6-diaza-5(2,6)-pyridina-1(1,4)-piperidina-2(1,2)-benzenacycloundecaphan-8-en-3-one (360 mg, 25% yield). LCMS (ESI-MS) m/z=535.1 [M+H]⁺.

Step 6. 14-methyl-2⁵-(methylsulfonyl)-5⁵-(1H-pyrazol-1-yl)-11-oxa-4,6-diaza-5(2,6)-pyridina-1(1,4)-piperidina-2(1,2)-benzenacycloundecaphan-8-en-3-one: Into a solution of 5⁵-bromo-1⁴-methyl-2⁵-(methylsulfonyl)-11-oxa-4,6-diaza-5(2,6)-pyridina-1(1,4)-piperidina-2(1,2)-benzenacycloundecaphan-8-en-3-one (350 mg, 0.655 mmol) in DMSO (4 mL) was added 1H-pyrazole (133.6 mg, 1.965 mmol), (1R,2R)—N¹,N²-dimethylcyclohexane-1,2-diamine (279 mg, 1.965 mmol), CuI (373.4 mg, 1.965 mmol) and K₃PO₄ (416.6 mg, 1.965 mmol). The resulting solution was heated to 100° C. and stirred for 3 hours. After cooling to room temperature, the reaction mixture was filtered and concentrated under vacuum. The residue was purified with silica gel column, eluted with EA:PE=1:1 to afford 1⁴-methyl-2⁵-(methylsulfonyl)-5⁵-(1H-pyrazol-1-yl)-11-oxa-4,6-diaza-5(2,6)-pyridina-1(1,4)-piperidina-2(1,2)-benzenacycloundecaphan-8-en-3-one (150 mg, 43% yield). LCMS (ESI-MS) m/z=523.2 [M+H]⁺.

Step 7. Compounds 153 and 154: Purification of 1⁴-methyl-2⁵-(methylsulfonyl)-5⁵-(1H-pyrazol-1-yl)-11-oxa-4,6-diaza-5(2,6)-pyridina-1(1,4)-piperidina-2(1,2)-benzenacycloundecaphan-8-en-3-one (150 mg) via Prep-chiral-SFC with the following conditions (Column: Xselect CSH Prep C18 OBD Column, 30*150 mm, 5 μm; Mobile Phase A: Water(0.1% FA), Mobile Phase B: ACN; Flow rate: 60 mL/min mL/min; Gradient (B %): 39% B to 69% B in 10 min; Wave Length: 254 nm/220 nm nm; RT1 (min): 8.3, 9.7 (min):) to afford:

First eluting isomer (Compound 153): (Z)-1⁴-methyl-2⁵-(methylsulfonyl)-5⁵-(1H-pyrazol-1-yl)-11-oxa-4,6-diaza-5(2,6)-pyridina-1(1,4)-piperidina-2(1,2)-benzenacycloundecaphan-8-en-3-one (10.9 mg, 72% yield).

Second eluting isomer (Compound 154): (E)-1⁴-methyl-2⁵-(methylsulfonyl)-5⁵-(1H-pyrazol-1-yl)-11-oxa-4,6-diaza-5(2,6)-pyridina-1(1,4)-piperidina-2(1,2)-benzenacycloundecaphan-8-en-3-one (7.7 mg, 5.1% yield).

### Compounds 155 and 156.

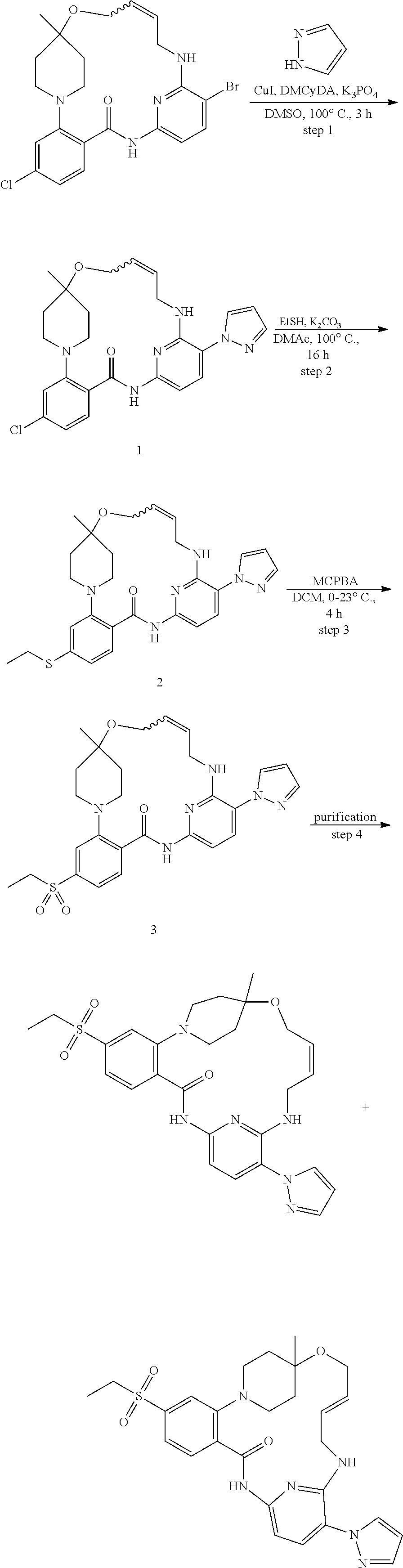

Step 1. 2⁵-chloro-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one: Into a mixture of 5⁵-bromo-2⁵-chloro-14-methyl-11-oxa-4,6-diaza-5(2,6)-pyridina-1(1,4)-piperidina-2(1,2)-benzenacycloundecaphan-8-en-3-one (120 mg, 0.24 mmol) in DMSO (3 mL) was added 1H-pyrazole (33 mg, 0.48 mmol), (1R,2R)—N¹,N²-dimethylcyclohexane-1,2-diamine (68.2 mg, 0.48 mmol), CuI (91.2 mg, 0.48 mmol) and K₃PO₄ (153 mg, 0.72 mmol) under nitrogen atmosphere. The resulting solution was heated to 100° C. and stirred for 4 hours. After cooling to room temperature, the reaction mixture was diluted with water and extracted with EA (3×100 mL). The combined organic layers were washed with brine (200 mL), dried over anhydrous Na₂SO₄, and concentrated under reduced pressure. The residue was purified by silica gel column chromatography, eluted with EA/PE (1:1) to afford 2⁵-chloro-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (100 mg, 85% yield). LCMS (ESI-MS) m/z=479.2 [M+H]⁺.

Step 2. 2⁵-(ethylthio)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one: Into a mixture of 2⁵-chloro-14-methyl-55-(1H-pyrazol-1-yl)-11-oxa-4,6-diaza-5(2,6)-pyridina-1(1,4)-piperidina-2(1,2)-benzenacycloundecaphan-8-en-3-one (90 mg, 0.19 mmol) in DMAc (3 mL) was added ethanethiol (118 mg, 1.90 mmol) and K₂CO₃ (79 mg, 0.57 mmol). The resulting solution was heated to 100° C. and stirred for 16 hours. After cooling to room temperature, the reaction mixture was diluted with water (100 mL) and extracted with EA (3×100 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:2) to afford 25-(ethylthio)-14-methyl-55-(1H-pyrazol-1-yl)-11-oxa-4,6-diaza-5(2,6)-pyridina-1(1,4)-piperidina-2(1,2)-benzenacycloundecaphan-8-en-3-one (80 mg, 84% yield). LCMS (ESI-MS) m/z=505.2 [M+H]⁺.

Step 3. 2⁵-(ethylsulfonyl)-14-methyl-54-(1H-pyrazol-1-yl)-11-oxa-4,6-diaza-1(1,4)-piperidina-2(1,2),5(1,3)-dibenzenacycloundecaphan-8-en-3-one: Into a mixture of 2⁵-(ethylthio)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (70 mg, 0.14 mmol) in DCM (3 mL) was added m-CPBA (48 mg, 0.28 mmol) at 0° C. The resulting solution was stirred for 1 hour at room temperature. Then, the reaction mixture was diluted with saturated sodium bicarbonate (50 mL) and extracted with EA (3×50 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:1) to afford 2⁵-(ethylthio)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (50 mg, 67% yield). LCMS (ESI-MS) m/z=537.1 [M+H]⁺.

Compounds 155 and 156: 2⁵-(ethylthio)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (50 mg) was separated by Prep-HPLC with the following conditions (Column: XBridge Prep OBD C18 Column, 30*150 mm, 5 μm; Mobile Phase A: Water(10 mmol/L NH4HCO3), Mobile Phase B: ACN; Flow rate: 60 mL/min mL/min; Gradient (B %): 43% B to 58% B in 10 min; Wave Length: 254 nm/220 nm nm; RT1 (min): 8.77; 9.88) to afford:

First eluting isomer (Compound 155): (Z)-2⁵-(ethylsulfonyl)-1⁴-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (6.0 mg, 12% yield). LCMS (ESI-MS).

Second eluting isomer (Compound 156): (E)-2⁵-(ethylsulfonyl)-1⁴-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (6.0 mg, 12% yield).

### Compounds 157 and 158.

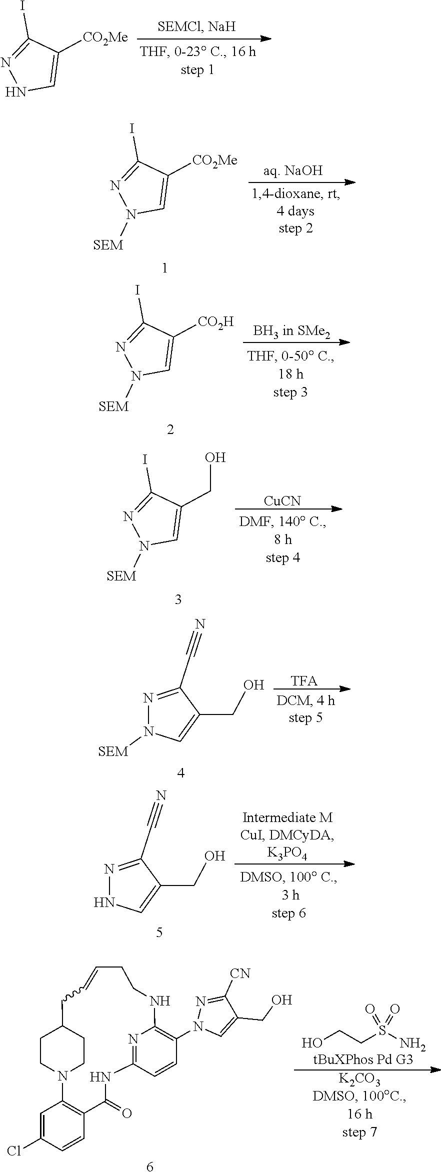

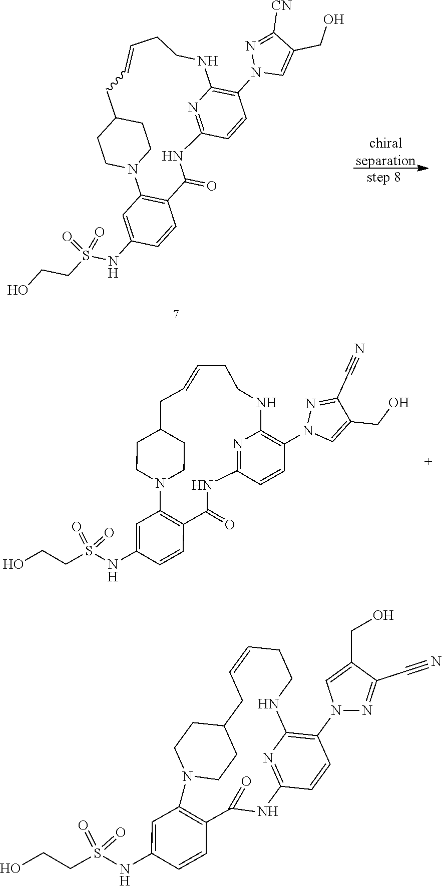 [SMILES: N#Cc1nn(-c2ccc3nc2NCCC=CCC2CCN(CC2)c2cc(NS(=O)(=O)CCO)ccc2C(=O)N3)cc1CO ; N#Cc1nn(-c2ccc3nc2NCC/C=C/CC2CCN(CC2)c2cc(NS(=O)(=O)CCO)ccc2C(=O)N3)cc1CO ; N#Cc1nn(-c2ccc3nc2NCC/C=C\CC2CCN(CC2)c2cc(NS(=O)(=O)CCO)ccc2C(=O)N3)cc1CO]

Step 1. ethyl 3-iodo-1-((2-(trimethylsilyl)ethoxy)methyl)-1H-pyrazole-4-carboxylate: A compound ethyl 3-iodo-1H-pyrazole-4-carboxylate (2.0 g, 7.5 mmol) was dissolved in tetrahydrofuran (80 mL) and sodium hydride (60% mineral oil dispersion, 0.42 g, 10.5 mmol) was added at 0° C. After stirring at room temperature for 1 hour, the reaction mixture was added 2-(trimethylsilyl)ethoxymethyl chloride (1.76 g, 10.5 mmol), continued stirring for 16 hours, and then a saturated brine (80 mL) was added to the reaction mixture, which was then extracted with ethyl acetate (80 mL×3), the organic phases were combined and washed with saturated brine (80 mL), the solvent was removed under reduced pressure, the residue was purified by silica gel column chromatography, eluted with EA:PE=1:5 to afford ethyl 3-iodo-1-((2-(trimethylsilyl)ethoxy)methyl)-1H-pyrazole-4-carboxylate (2.5 g, 84% yield). LCMS (ESI-MS) m/z=397.0 [M+H]⁺.

Step 2. 3-iodo-1-((2-(trimethylsilyl)ethoxy)methyl)-1H-pyrazole-4-carboxylic acid: Ethyl 3-iodo-1-((2-(trimethylsilyl)ethoxy)methyl)-1H-pyrazole-4-carboxylate (2.5 g, 6.3 mmol) and 3N aqueous sodium hydroxide (5.5 mL, 16.5 mmol) in 1,4-dioxane (22 mL) was stirred for four days. The solution was brought to pH=3 and extracted with CH₂Cl₂. The combined organic extracts were dried over Na₂SO₄, filtered, and concentrated under reduced pressure to afford 3-iodo-1-((2-(trimethylsilyl)ethoxy)methyl)-1H-pyrazole-4-carboxylic acid (1.5 g, 64% yield). LCMS (ESI-MS) m/z=369.0 [M+H]⁺.

Step 3. (3-iodo-1-((2-(trimethylsilyl)ethoxy)methyl)-1H-pyrazol-4-yl)methanol: Borane dimethyl sulfide complex 10M (1.49 mL, 14.9 mmol) was slowly added to 3-iodo-1-((2-(trimethylsilyl)ethoxy)methyl)-1H-pyrazole-4-carboxylic acid (1.2 g, 3.3 mmol) in THF (12 mL) at 0° C. After stirring for 30 minutes, the solution was placed into a preheated oil bath at 50° C. After stirring for 18 hours, the solution was cooled to 0° C. and ice was added. After stirring for 30 minutes, saturated aqueous NH₄Cl was added and stirred for an additional 30 minutes. The solution was extracted with CH₂Cl₂ and the combined organic extracts were dried over Na₂SO₄, filtered, and concentrated under reduced pressure to afford (3-iodo-1-((2-(trimethylsilyl)ethoxy)methyl)-1H-pyrazol-4-yl)methanol (1 g, 87% yield). LCMS (ESI-MS) m/z=355.0 [M+H]⁺.

Step 4. 4-(hydroxymethyl)-1-((2-(trimethylsilyl)ethoxy)methyl)-1H-pyrazole-3-carbonitrile: Copper(I) Cyanide (303 mg, 3.38 mmol) was added to a mixture of (3-iodo-1-((2-(trimethylsilyl)ethoxy)methyl)-1H-pyrazol-4-yl)methanol (800 mg, 2.25 mmol) in DMF (10 mL) under nitrogen atmosphere. The mixture was stirred at 140° C. for 8 hours. After cooling to room temperature, the reaction mixture was diluted with water (10 mL), extracted with EA (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with EA:PE=1:1 to afford 4-(hydroxymethyl)-1-((2-(trimethylsilyl)ethoxy)methyl)-1H-pyrazole-3-carbonitrile (150 mg, 26% yield). LCMS (ESI-MS) m/z=254.15 [M+H]⁺.

Step 5. 4-(hydroxymethyl)-1H-pyrazole-3-carbonitrile: TFA (1 mL, 1.49 mmol) was added to a mixture of 4-(hydroxymethyl)-1-((2-(trimethylsilyl)ethoxy)methyl)-1H-pyrazole-3-carbonitrile (150 mg, 0.59 mmol) in DCM (3 mL). After stirring at room temperature for 4 hours, the mixture was diluted with water (3 mL), extracted with EA (3×1 mL). The combined organic layers were washed with brine (2×3 mL), dried over anhydrous Na₂SO₄. After filtration, the filtrate was concentrated under reduced pressure to afford the crude 4-(hydroxymethyl)-1H-pyrazole-3-carbonitrile (70 mg, 96% yield). LCMS (ESI-MS) m/z=124.15 [M+H]⁺.

Step 6. 1-(2⁵-chloro-3-oxo-4,6-diaza-5(2,6)-pyridina-1(1,4)-piperidina-2(1,2)-benzenacycloundecaphan-9-en-5⁵-yl)-4-(hydroxymethyl)-1H-pyrazole-3-carbonitrile: Into a solution of 5⁵-bromo-2⁵-chloro-4,6-diaza-5(2,6)-pyridina-1(1,4)-piperidina-2(1,2)-benzenacycloundecaphan-9-en-3-one (90 mg, 189 μmol) in DMSO (1 mL) was added 4-(hydroxymethyl)-1H-pyrazole-3-carbonitrile (35 mg, 284 μmol), (1R,2R)—N¹,N²-dimethylcyclohexane-1,2-diamine (80.9 mg, 569 μmol), CuI (108.5 mg, 569 gmol) and K₃PO₄ (120.9 mg, 569 μmol). The resulting mixture was heated to 100° C. and stirred for 3 hours. After cooling to room temperature, the reaction mixture was diluted with water (1 mL), extracted with EA (3×1 mL). The combined organic layers were washed brine (2×1 mL), dried with anhydrous Na₂SO₄, filtered and evaporated. The crude was purified with silica gel column, eluted with EA:PE=1:2 to afford the desired product 1-(2⁵-chloro-3-oxo-4,6-diaza-5(2,6)-pyridina-1(1,4)-piperidina-2(1,2)-benzenacycloundecaphan-9-en-5⁵-yl)-4-(hydroxymethyl)-1H-pyrazole-3-carbonitrile (15 mg, 15% yield). LCMS (ESI-MS) m/z=518.2 [M+H]⁺.

Step 7. N-(5⁵-(3-cyano-4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)-2-hydroxyethane-1-sulfonamide: t-BuXphos Pd G3 (9.2 mg, 11.6 gmol) was added to a mixture of 1-(2⁵-chloro-3-oxo-4,6-diaza-5(2,6)-pyridina-1(1,4)-piperidina-2(1,2)-benzenacycloundecaphan-9-en-5⁵-yl)-4-(hydroxymethyl)-1H-pyrazole-3-carbonitrile (30 mg, 58 gmol), 2-hydroxyethane-1-sulfonamide (21 mg, 0.174 mmol), K₂CO₃ (24 mg, 0.174 mmol) in DMSO (1 mL) under nitrogen atmosphere. The mixture was stirred at 100° C. for 16 hours. After cooling to room temperature, the reaction mixture was diluted with water (1 mL), extracted with EA (3×1 mL). The combined organic layers were washed with brine (2×1 mL), dried over anhydrous Na₂SO₄. After filtration, the filtrate was concentrated under reduced pressure. The residue was purified by Prep-HPLC with the following conditions (Column: XBridge Prep OBD C18 Column, 30*150 mm, 5 μm; Mobile Phase A: Water(10 mmol/LNH₄HCO₃), Mobile Phase B: ACN; Flow rate: 60 mL/min; Gradient (B %): 30% B to 60% B in 10 min; Wave Length: 254 nm/220 nm; RT1 (min): 8.02) to afford (E)-N-(5⁵-(3-cyano-4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)-2-hydroxyethane-1-sulfonamide (5.0 mg, 14% yield). LCMS (ESI-MS) m/z=607.2 [M+H]⁺.

Step 8. Compounds 157 and 158: N-(5⁵-(3-cyano-4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)-2-hydroxyethane-1-sulfonamide (5.0 mg, 8 gmol) was separated by Prep-Chiral HPLC with the following conditions (Column: (R, R)-WHELK-01-Kromasil, 2.12*25 cm, 5 μm; Mobile Phase A: Hex--HPLC, Mobile Phase B: MeOH:DCM=1:1--HPLC; Flow rate: 20 mL/min; Gradient (B %): isocratic 80; Wave Length: 254/220 nm; RT1 (min): 3.904; RT2 (min): 5.856; Sample Solvent: EtOH--HPLC; Injection Volume: 1.0 mL) to afford:

First eluting isomer (Compound 157): (E)-N-(5⁵-(3-cyano-4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)-2-hydroxyethane-1-sulfonamide (2.3 mg, 46% yield).

Second eluting isomer (Compound 158): (Z)—N-(5⁵-(3-cyano-4-(hydroxymethyl)-1H-pyrazol-1-yl)-3-oxo-4,6-diaza-5(2,6)-pyridina-1(1,4)-piperidina-2(1,2)-benzenacycloundecaphan-9-en-2⁵-yl)-2-hydroxyethane-1-sulfonamide (0.3 mg, 6% yield).

### Compounds 159 and 160.

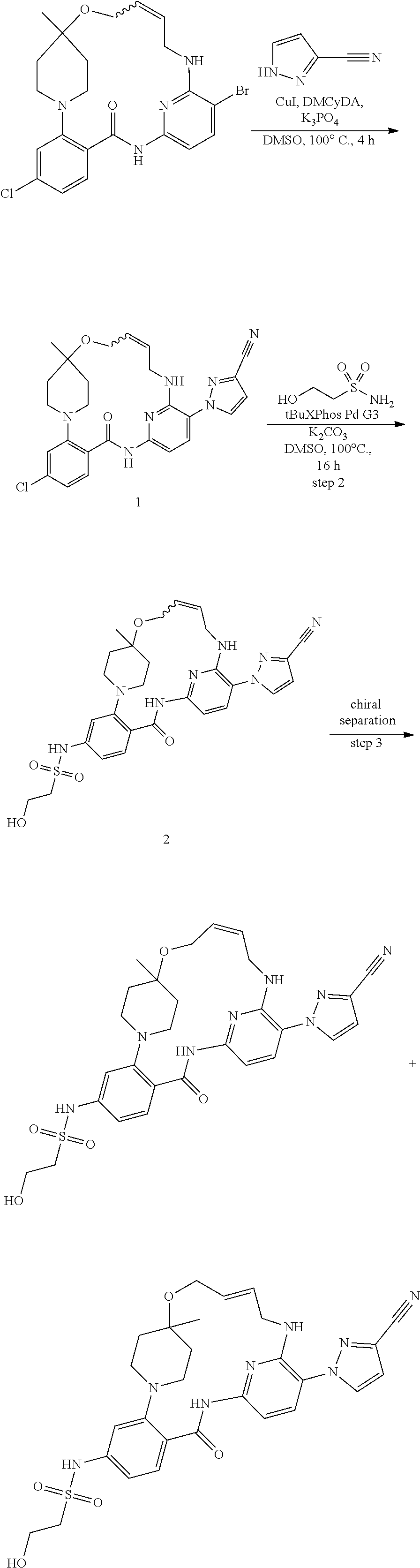

Step 1. 1-(2⁵-chloro-14-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-3-carbonitrile: Into a solution of 5⁵-bromo-2⁵-chloro-1⁴-methyl-11-oxa-4,6-diaza-5(2,6)-pyridina-1(1,4)-piperidina-2(1,2)-benzenacycloundecaphan-8-en-3-one (100 mg, 204 μmol) in DMSO (1 mL) was added 1H-pyrazole-3-carbonitrile (56.9 mg, 0.61 mmol), (1R,2R)—N¹,N²-dimethylcyclohexane-1,2-diamine (87.0 mg, 0.61 mmol), K₃PO₄ (129.9 mg, 0.61 mmol), CuI (116.6 mg, 0.61 mmol). The resulting mixture was heated to 100° C. and stirred for 4 hours. After cooling to room temperature, the reaction mixture was diluted with water (1 mL), extracted with EA (3×1 mL). The combined organic layers were washed brine (3×1 mL), dried with anhydrous Na₂SO₄, filtered and evaporated to give a crude. The crude was purified with silica gel column, eluted with EA:PE=1:2 to afford the desired product 1-(2⁵-chloro-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-3-carbonitrile (58 mg, 56% yield). LCMS (ESI-MS) m/z=504.2 [M+H]⁺.

Step 2. N-(5⁵-(3-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide: t-BuXphos Pd G3 (15.1 mg, 19 μmol) was added to a mixture of 1-(2⁵-chloro-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-3-carbonitrile (48 mg, 95 μmol), 2-hydroxyethane-1-sulfonamide (35.7 mg, 0.285 mmol), K₂CO₃ (39.4 mg, 0.285 mmol) in DMSO (1 mL) under nitrogen atmosphere. The mixture was stirred at 100° C. for 16 hours. After cooling to room temperature, the reaction mixture was diluted with water (1 mL), extracted with EA (3×1 mL). The combined organic layers were washed with brine (2×1 mL), dried over anhydrous Na₂SO₄. After filtration, the filtrate was concentrated under reduced pressure. The residue was purified by silica gel column chromatography, eluted with EA:PE=1:1 to afford N-(5⁵-(3-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide (27 mg, 47% yield). LCMS (ESI-MS) m/z=593.25 [M+H]⁺.

Step 3. Compounds 159 and 160: N-(5⁵-(3-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide (20 mg, 34 μmol) was separated by Prep-HPLC with the following conditions (Column: Xselect CSH Prep C18 OBD Column, 30*150 mm, 5 μm; Mobile Phase A: Water(0.1% FA), Mobile Phase B: ACN; Flow rate: 60 mL/min mL/min; Gradient (B %): 29% B to 59% B in 10 min; Wave Length: 254 nm/220 nm nm; RT1 (min): 8.8, 9.8 (min):) to afford:

First eluting isomer (Compound 159): (Z)—N-(5⁵-(3-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide (0.9 mg, 4.5% yield).

Second eluting isomer (Compound 160): (E)-N-(5⁵-(3-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide (0.6 mg, 3.0% yield).

### Compounds 161 and 162.

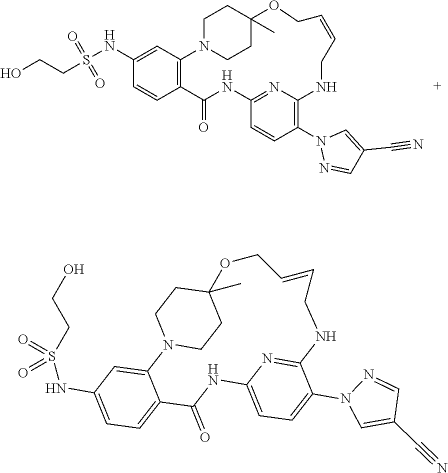 [SMILES: CC12CCN(CC1)c1cc(NS(=O)(=O)CCO)ccc1C(=O)Nc1ccc(-n3cc(C#N)cn3)c(n1)NC/C=C\CO2 ; CC12CCN(CC1)c1cc(NS(=O)(=O)CCO)ccc1C(=O)Nc1ccc(-n3cc(C#N)cn3)c(n1)NC/C=C/CO2]

Compounds 161 and 162 were prepared according to the procedure outlined in Compounds 159 and 160 with 1H-pyrazole-4-carbonitrile used in Step 1. The procedure for chiral preparative HPLC in step 3 is as follows: N-(5⁵-(4-cyano-1H-pyrazol-1-yl)-1⁴-methyl-3-oxo-1′-oxa-4,6-diaza-5(2,6)-pyridina-1(1,4)-piperidina-2(1,2)-benzenacycloundecaphan-8-en-2⁵-yl)-2-hydroxyethane-1-sulfonamide (30 mg) was separated by Prep-Chiral-HPLC with the following conditions (Column: (R, R)-WHELK-01-Kromasil, 2.12*25 cm, 5 μm; Mobile Phase A: Hex--HPLC, Mobile Phase B: EtOH:DCM=1:1--HPLC; Flow rate: 20 mL/min; Gradient (B %): isocratic 50; Wave Length: 254/220 nm; RT1 (min): 13.563; RT2 (min): 16.622; Sample Solvent: EtOH--HPLC; Injection Volume: 0.9 mL; Number Of Runs: 3) to afford:

First eluting isomer (Compound 161): (Z)—N-(5⁵-(4-cyano-1H-pyrazol-1-yl)-1⁴-methyl-3-oxo-1′-oxa-4,6-diaza-5(2,6)-pyridina-1(1,4)-piperidina-2(1,2)-benzenacycloundecaphan-8-en-2⁵-yl)-2-hydroxyethane-1-sulfonamide (3.3 mg, 11% yield).

Second eluting isomer (Compound 162): (E)-N-(5⁵-(4-cyano-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)-2-hydroxyethane-1-sulfonamide (5.8 mg, 19% yield).

### Compounds 163 and 164.

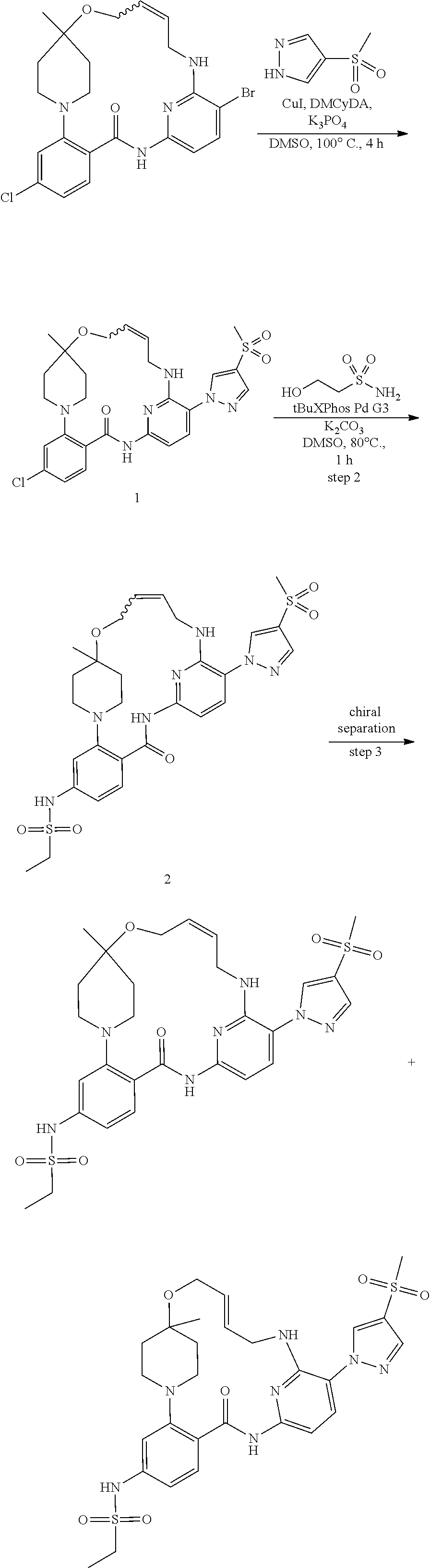

Step 1. 2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one: CuI (176.7 mg, 0.93 mmol) was added to a mixture of 5⁵-bromo-2⁵-chloro-1⁴-methyl-11-oxa-4,6-diaza-5(2,6)-pyridina-1(1,4)-piperidina-2(1,2)-benzenacycloundecaphan-8-en-3-one (150 mg, 0.31 mmol), 4-(methylsulfonyl)-1H-pyrazole (226.3 mg, 1.55 mmol), (1R,2R)—N¹,N²-dimethylcyclohexane-1,2-diamine (132 mg, 0.93 mmol) and K₃PO₄ (197.2 mg, 0.93 mmol) in DMSO (2 mL) under nitrogen atmosphere. The mixture was stirred at 100° C. overnight. The reaction was purified by silica gel column chromatography, eluted with PE/EA (1:1) to afford 2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (60 mg, 35%). LCMS (ESI-MS) m/z=557.3 [M+H]⁺.

Step 2. N-(1⁴-methyl-5⁵-(4-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide: tBuXPhos Pd G3 (13.8 mg, 0. 017 mmol) was added to a mixture of 2⁵-chloro-1⁴-methyl-5⁵-(4-(methylsulfonyl)-1H-pyrazol-1-yl)-11-oxa-4,6-diaza-5(2,6)-pyridina-1(1,4)-piperidina-2(1,2)-benzenacycloundecaphan-8-en-3-one (55 mg, 0.087 mmol), ethanesulfonamide (28.4 mg, 0.261 mmol) and K₂CO₃ (36.0 mg, 0.261 mmol) in DMSO (1 mL) under nitrogen atmosphere. The mixture was stirred at 80° C. for 1 hour. The reaction was purified by silica gel column chromatography, eluted with PE/EA (1:1) to afford N-(1⁴-methyl-5⁵-(4-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (40 mg, 64%). LCMS (ESI-MS) m/z=630.2 [M+H]⁺.

Step 3. Compounds 163 and 164: N-(1⁴-methyl-5⁵-(4-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (40 mg) was separated by Prep-chiral-SFC with the following conditionsColumn: (R, R)WhelkOl; Mobile Phase A: Hex: (EtOH:DCM=1:1)=50:50; Flow rate: 1 mL/min mL/min; Gradient (B %): isocratic; Injection Volume: 1 mL) to afford:

First eluting isomer (Compound 163): (Z)—N-(1⁴-methyl-5⁵-(4-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4.7 mg, 12% yield).

Second eluting isomer (Compound 164): (E)-N-(1⁴-methyl-5⁵-(4-(methylsulfonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4.5 mg, 11% yield).

Compounds 165 and 166 were prepared according to the procedure outlined in Compounds 163 and 164 using (1H-pyrazol-3-yl)methanol in Step 1. The desired products were purified by preparative-Chiral-HPLC with the following conditions (Column: CHIRALPAK IG, 2*25 cm, 5 μm; Mobile Phase A: Hex--HPLC, Mobile Phase B: MeOH:DCM=1:1--HPLC; Flow rate: 20 mL/min; Gradient (B %): isocratic 40; Wave Length: 254/220 nm; RT1 (min): 6.78; RT2 (min): 8.903; Sample Solvent: EtOH--HPLC; Injection Volume: 0.8 mL; Number Of Runs: 6) to afford:

First eluting isomer (Compound 165): (Z)—N-(5⁵-(3-(hydroxymethyl)-1H-pyrazol-1-yl)-1⁴-methyl-3-oxo-1′-oxa-4,6-diaza-5(2,6)-pyridina-1(1,4)-piperidina-2(1,2)-benzenacycloundecaphan-8-en-2⁵-yl)ethanesulfonamide (0.3 mg, 1.5% yield).

Second eluting isomer (Compound 166): (E)-N-(5⁵-(3-(hydroxymethyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.5 mg, 7.5% yield).

Compounds 167 and 168 were prepared according to the procedure outlined for Compounds 163 and 164 using (1H-pyrazol-4-yl)methanol in step 1. The procedure for chiral preparative HPLC in Step 3 is as follows: N-(5⁵-(4-(hydroxymethyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (30 mg) was separated by Prep-chiral-SFC with the following conditions (Column: CHIRALPAK IG, 2*25 cm, 5 μm; Mobile Phase A: Hex--HPLC, Mobile Phase B: MeOH:DCM=1:1--HPLC; Flow rate: 20 mL/min; Gradient (B %): isocratic 40; Wave Length: 254/220 nm; RT1 (min): 7.853; RT2 (min): 11.256; Sample Solvent: EtOH--HPLC; Injection Volume: 0.6 mL; Number Of Runs: 4) to afford:

First eluting isomer (Compound 167): (Z)—N-(5⁵-(4-(hydroxymethyl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.0 mg, 6.7% yield). LCMS (ESI-MS) m/z=582.2 [M+H]⁺.

Second eluting isomer (Compound 168): (E)-N-(5⁵-(4-(hydroxymethyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.2 mg, 11% yield). LCMS (ESI-MS) m/z=582.2 [M+H]⁺.

Compounds 169 and 170 were prepared according to the procedures outlined for Compounds 163 and 164 using 1H-pyrazole in Step 1. The procedure for chiral preparative HPLC in Step 3 is as follows: N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (25 mg, 45 μmol) was dissolved with MeOH (1 mL) and purified by PREP-HPLC with the following conditions: Column: Xbridge Prep OBD Shield RP18, 30*150 mm, 5 μm; Mobile Phase A: Water(10 mmol/L NH4HCO3), Mobile Phase B: ACN; Flow rate: 60 mL/min mL/min; Gradient (B %): 42% B to63% B in 10 min; Wave Length: 254 nm/220 nm nm; RT1 (min): 7.91; 8.55 to afford:

First eluting isomer (Compound 169): (Z)—N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (1.3 mg, 5.2% yield).

Second eluting isomer (Compound 170): (E)-N-(1⁴-methyl-3-oxo-5⁵-(1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (3.6 mg, 14% yield).

Compounds 171 and 172 were prepared using Steps 2 and 3 of the procedure outlined for Compounds 137 and 138 using Intermediate K in Step 2. The desired products were purified using purified by preparative HPLC with the following conditions (Column: XBridge Prep OBD C18 Column, 30*150 mm, 5 μm; Mobile Phase A: Water(10 mmol/LNH4HCO3), Mobile Phase B: ACN; Flow rate: 60 mL/min mL/min; Gradient (B %): 40% B to70% B in 10 min; Wave Length: 254 nm/220 nm nm; RT1 (min): 8.5; 9.5) to afford:

First eluting isomer (Compound 171): (Z)-2⁵-(3,3-dioxido-1,3,4-oxathiazinan-4-yl)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (2.4 mg, 9.6% yield).

Second eluting isomer (Compound 172): (E)-2⁵-(3,3-dioxido-1,3,4-oxathiazinan-4-yl)-14-methyl-5⁵-(1H-pyrazol-1-yl)-11-oxa-4,6-diaza-5(2,6)-pyridina-1(1,4)-piperidina-2(1,2)-benzenacycloundecaphan-8-en-3-one (2.2 mg, 8.8% yield)

Compounds 173 and 174 were prepared according to the procedures outlined for Compounds 155 and 156 using 1H-pyrazole-4-carbonitrile in step 1. The desired products were purified under the following conditions (Column: XBridge Prep OBD C18 Column, 30*150 mm, 5 μm; Mobile Phase A: Water(10 mmol/L NH4HCO3), Mobile Phase B: ACN; Flow rate: 60 mL/min mL/min; Gradient (B %): 45% B to 60% B in 10 min; Wave Length: 254 nm/220 nm nm; RT1 (min): 9.13) to afford:

First eluting isomer (Compound 173): (Z)-1-(2⁵-(ethylsulfonyl)-1⁴-methyl-3-oxo-1′-oxa-4,6-diaza-5(2,6)-pyridina-1(1,4)-piperidina-2(1,2)-benzenacycloundecaphan-8-en-5⁵-yl)-1H-pyrazole-4-carbonitrile (2.6 mg, 10% yield).

Second eluting isomer (Compound 174): (E)-1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazole-4-carbonitrile (2.0 mg, 8.0% yield).

### Compounds 175 and 176.

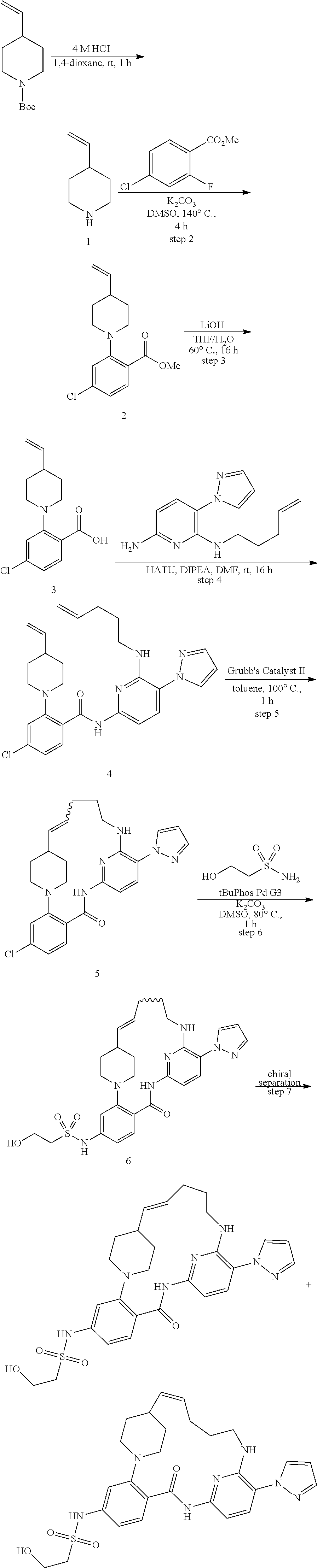

Step 1. 4-vinylpiperidine: 4 M HCl in dioxane (80 mL) was added to a stirred mixture of tert-butyl 4-allylpiperidine-1-carboxylate (3.0 g, 14.21 mmol) in dioxane (30 mL). The resulting mixture was stirred at room temperature for 1 hour and concentrated to afford the crude product 4-vinylpiperidine (1.5 g crude). LCMS (ESI-MS) m/z=112.1 [M+H]⁺.

Step 2. Methyl 4-chloro-2-(4-vinylpiperidin-1-yl)benzoate: K₂CO₃ (5.21 g, 37.8 mmol) was added to a mixture of 4-allylpiperidine (1.4 g, 12.6 mmol) and methyl 4-chloro-2-fluorobenzoate (2.37 g, 12.6 mmol) in DMSO (40 mL). The resulting mixture was heated to 140° C. and stirred for 4 hours. After cooling to room temperature, the reaction mixture was diluted with water (400 mL) and extracted with EA (3×300 mL). The combined organic layers were washed with brine (500 mL), dried over anhydrous Na₂SO₄, filtered and concentrated under vacuum to afford the crude product. The residue was purified by silica gel column chromatography, eluted with PE:EA=5:1 to afford methyl 4-chloro-2-(4-vinylpiperidin-1-yl)benzoate (3.3 g, 94% yield). LCMS (ESI-MS) m/z=280.0 [M+H]⁺.

Step 3. 4-chloro-2-(4-vinylpiperidin-1-yl)benzoic acid: LiOH (1.38 g, 57 mmol) was added to a mixture of 4-chloro-2-(4-vinylpiperidin-1-yl)benzoate (1.6 g, 5.7 mmol) in THF (15 mL) and H₂O (5 mL). The resulting mixture was stirred at room temperature for 16 hours. After cooling to room temperature, the reaction mixture was diluted with water (400 mL) and extracted with EA (3×400 mL). The combined organic layers were washed with brine (300 mL), dried over anhydrous Na₂SO₄, filtered and concentrated under vacuum to afford 4-chloro-2-(4-vinylpiperidin-1-yl)benzoic acid (1.5 g crude). LCMS (ESI-MS) m/z=266.0 [M+H]⁺.

Step 4. 4-chloro-N-(6-(pent-4-en-1-ylamino)-5-(1H-pyrazol-1-yl)pyridin-2-yl)-2-(4-vinylpiperidin-1-yl)benzamide: Into a solution of 4-chloro-2-(4-vinylpiperidin-1-yl)benzoic acid (381.7 mg, 1.44 mmol) in DMF (40 mL) was added N²-(pent-4-en-1-yl)-3-(1H-pyrazol-1-yl)pyridine-2,6-diamine (350 mg, 1.44 mmol), HATU (452.1 mg, 2.88 mmol) and DIPEA (557.3 mg, 4.32 mmol). The resulting mixture was stirred at room temperature for 16 hours. Then, the reaction mixture was diluted with water (400 mL) and extracted with EA (3×400 mL). The combined organic layers were washed with brine (300 mL), dried with anhydrous Na₂SO₄, filtered and concentrated under vacuum. The residue was purified with silica gel column, eluted with PE:EA=5:1 to afford 4-chloro-N-(6-(pent-4-en-1-ylamino)-5-(1H-pyrazol-1-yl)pyridin-2-yl)-2-(4-vinylpiperidin-1-yl)benzamide (460 mg, 65% yield). LCMS (ESI-MS) m/z=491.2 [M+H]⁺.

Step 5. 2⁵-chloro-5⁵-(1H-pyrazol-1-yl)-4,6-diaza-5(2,6)-pyridina-1(1,4)-piperidina-2(1,2)-benzenacycloundecaphan-10-en-3-one: 4-chloro-N-(6-(pent-4-en-1-ylamino)-5-(1H-pyrazol-1-yl)pyridin-2-yl)-2-(4-vinylpiperidin-1-yl)benzamide (400 mg, 8.16 mmol) was added to a stirred mixture of Benzylidene(1,3-bis(2,4,6-trimethylpheyl-2-imidazolidinylidene)dichloro(tricyclohexylphosphine) ruthenium (1.39 g, 1.63 mmol) in toluene (25 mL). The resulting mixture was stirred for 1 hour at 100° C. The resulting mixture was concentrated under reduced pressure. The residue was purified by silica gel column chromatography, eluted with PE:EA (5:1) to afford 2⁵-chloro-5⁵-(1H-pyrazol-1-yl)-4,6-diaza-5(2,6)-pyridina-1(1,4)-piperidina-2(1,2)-benzenacycloundecaphan-10-en-3-one (120 mg, 32% yield). LCMS (ESI-MS) m/z=463.8 [M+H]⁺.

Step 6. 2-hydroxy-N-(3-oxo-5⁵-(1H-pyrazol-1-yl)-4,6-diaza-5(2,6)-pyridina-1(1,4)-piperidina-2(1,2)-benzenacycloundecaphan-10-en-2⁵-yl)ethane-1-sulfonamide: tBu-XPhos Pd G3 (34.9 mg, 0.044 mmol) was added to a mixture of 2⁵-chloro-5⁵-(1H-pyrazol-1-yl)-4,6-diaza-5(2,6)-pyridina-1(1,4)-piperidina-2(1,2)-benzenacycloundecaphan-10-en-3-one (100 mg, 0.22 mmol), 2-hydroxyethane-1-sulfonamide (81.2 mg, 0.66 mmol) and K₂CO₃ (89.6 mg, 0.66 mmol) in DMSO (2 mL) under nitrogen atmosphere. The mixture was stirred at 80° C. for 1 hour. The reaction was purified by silica gel column chromatography, eluted with PE/EA (1:1) to afford 2-hydroxy-N-(3-oxo-5⁵-(1H-pyrazol-1-yl)-4,6-diaza-5(2,6)-pyridina-1(1,4)-piperidina-2(1,2)-benzenacycloundecaphan-10-en-2⁵-yl)ethane-1-sulfonamide (70 mg, 58%). LCMS (ESI-MS) m/z=552.2 [M+H]⁺.

Step 7. Compounds 175 and 176: 2-hydroxy-N-(3-oxo-5⁵-(1H-pyrazol-1-yl)-4,6-diaza-5(2,6)-pyridina-1(1,4)-piperidina-2(1,2)-benzenacycloundecaphan-10-en-2⁵-yl)ethane-1-sulfonamide (70 mg) was separated by Prep-chiral-SFC with the following conditions (Column: CHIRALPAK IG, 2*25 cm, 5 μm; Mobile Phase A: Hex--HPLC, Mobile Phase B: EtOH:DCM=1:1--HPLC; Flow rate: 20 mL/min; Gradient (B %): isocratic 50; Wave Length: 254/220 nm; RT1 (min): 5.736; RT2 (min): 7.895; Sample Solvent: EtOH--HPLC; Injection Volume: 1.0 mL; Number Of Runs: 5) to afford:

First eluting isomer (Compound 175): (E)-2-hydroxy-N-(3-oxo-5⁵-(1H-pyrazol-1-yl)-4,6-diaza-5(2,6)-pyridina-1(1,4)-piperidina-2(1,2)-benzenacycloundecaphan-10-en-2⁵-yl)ethane-1-sulfonamide (14.2 mg, 20% yield). LCMS (ESI-MS) m/z=552.3 [M+H]⁺.

Second eluting isomer (Compound 176): (Z)-2-hydroxy-N-(3-oxo-5⁵-(1H-pyrazol-1-yl)-4,6-diaza-5(2,6)-pyridina-1(1,4)-piperidina-2(1,2)-benzenacycloundecaphan-10-en-2⁵-yl)ethane-1-sulfonamide (3.4 mg, 4.9% yield). LCMS (ESI-MS) m/z=552.2 [M+H]⁺.

### Compounds 177 and 178.

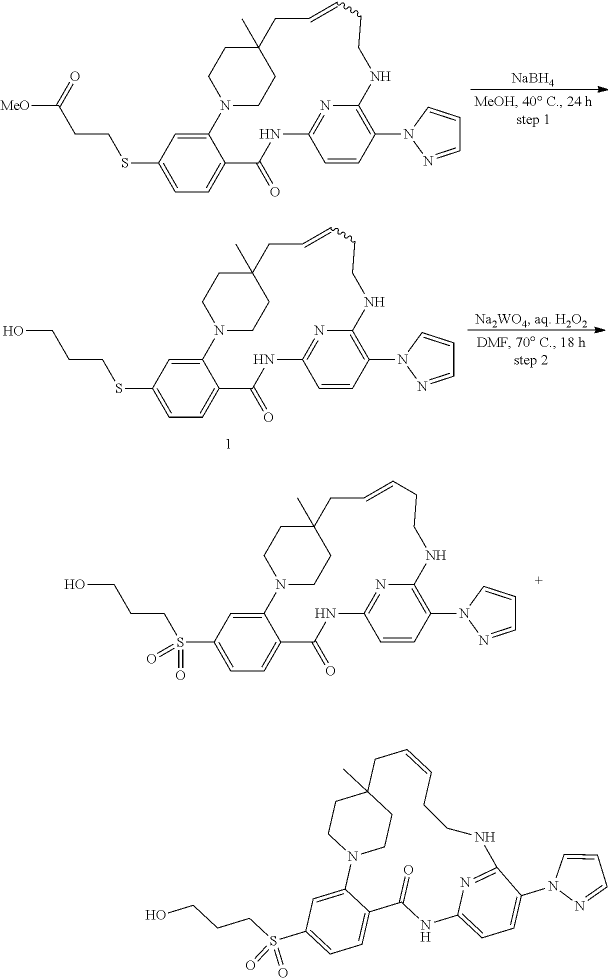

Step 1. 2⁵-((3-hydroxypropyl)thio)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one: To a vial was added methyl 3-((14-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)thio)propanoate (100 mg, 178 μmol), NaBH₄ (33.7 mg, 892 μmol), and MeOH (4.0 mL). The reaction mixture was heated to 50° C. Additional portions of NaBH₄ (33.7 mg, 892 μmol) were added every 30 min (10×). After 24 hour the reaction was quenched with 1M HCl and extracted with EtOAc (3×). The combined organic layers were washed with brine, dried over MgSO₄, and concentrated. Purification via Isco column chromatography (0-80% EtOAc on heptane) provided 25-((3-hydroxypropyl)thio)-14-methyl-5⁵-(1H-pyrazol-1-yl)-4,6-diaza-5(2,6)-pyridina-1(1,4)-piperidina-2(1,2)-benzenacycloundecaphan-9-en-3-one (51 mg, 54% yield).

Step 2. Compounds 177 and 178: To a vial was added 2⁵-((3-hydroxypropyl)thio)-14-methyl-5⁵-(1H-pyrazol-1-yl)-4,6-diaza-5(2,6)-pyridina-1(1,4)-piperidina-2(1,2)-benzenacycloundecaphan-9-en-3-one (50 mg, 94 μmol,) DMF (1.0 mL), and Sodium dioxido(dioxo)tungsten hydrate (2:1:2) (0.2 M in water) (3.10 mg, 47 μL, 9.4 μmol). The reaction was heated to 70° C. with stirring for 1 minute, then H₂O₂(128 mg, 115 μL, 50% Wt, 1.88 mmol) was added directly. After 18 hour the reaction was quenched with 0.2 M aq. Na₂S₂O₃ and aq. NaHCO₃ and extracted with EtOAc (3×). The combined organic layers were washed with brine, dried over MgSO₄, and concentrated. Purification via Isco column chromatography (0-100% EtOAc in heptane) followed by purification via preparative HPLC (10-100% MeCN in water with 0.1% TFA) afforded:

First eluting isomer (Compound 177): (E)-2⁵-((3-hydroxypropyl)sulfonyl)-14-methyl-5⁵-(1H-pyrazol-1-yl)-4,6-diaza-5(2,6)-pyridina-1(1,4)-piperidina-2(1,2)-benzenacycloundecaphan-9-en-3-one (2.8 mg, 4.5% yield).

Second eluting isomer (Compound 178): (Z)-2⁵-((3-hydroxypropyl)sulfonyl)-14-methyl-5⁵-(1H-pyrazol-1-yl)-4,6-diaza-5(2,6)-pyridina-1(1,4)-piperidina-2(1,2)-benzenacycloundecaphan-9-en-3-one (2.2 mg, 4.2% yield).

### Compound 179.

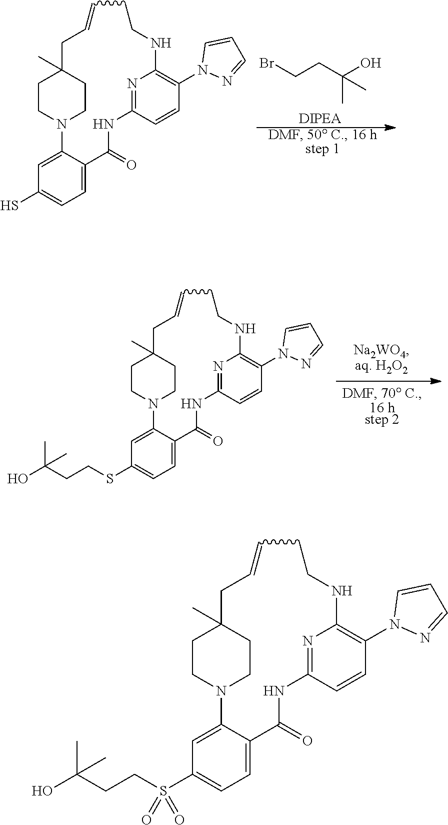

Step 1. 2⁵-((3-hydroxy-3-methylbutyl)thio)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one: To a vial was added 2-butanol, 4-bromo-2-methyl- (10.0 mg, 60.0 μmol) and followed by a solution of 2⁵-mercapto-14-methyl-5⁵-(1H-pyrazol-1-yl)-4,6-diaza-5(2,6)-pyridina-1(1,4)-piperidina-2(1,2)-benzenacycloundecaphan-9-en-3-one (23.7 mg, 50.0 μmol) and DIPEA (15.5 mg, 20.9 μL, 120 μmol) in DMF (250 μL) under a nitrogen atmosphere. The reaction mixture was then sealed and heated to 50° C. After 16 hour, reaction mixture was cooled to rt and used for the next step without further purification.

Step 2. Compound 179: To the crude reaction mixture from the previous step was added Sodium dioxido(dioxo)tungsten hydrate (2:1:2) (0.03 M in DMF) (1.6 mg, 0.17 mL, 5.0 μmol) followed by H₂O₂(0.10 g, 92 μL, 50% Wt, 1.5 mmol). The reaction vial was sealed and heated to 70° C. After 16 hour the reaction was quenched with 0.2 M aq. Na₂S₂O₃ and aq. NaHCO₃ and filtered. Purification via Prep-SFC with the following conditions: Columns: 2-Ethylpyridine, HA-morpholine, Diol, or 3,5 Dihydroxyphenyl, Mobile Phase A: C₀₂, Mobile Phase B: Methanol; Flow rate: 60 mL/min; Gradient (B %): 5-60% over 5.5 minutes provided 25-((3-hydroxy-3-methylbutyl)sulfonyl)-14-methyl-5⁵-(1H-pyrazol-1-yl)-4,6-diaza-5(2,6)-pyridina-1(1,4)-piperidina-2(1,2)-benzenacycloundecaphan-9-en-3-one (3.1 mg, 10% yield).

Compound 180 was prepared according to the procedure outlined for Compound 179 using (S)-4-chloro-3-hydroxybutanenitrile in step 1 to provide (S)-3-hydroxy-4-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)butanenitrile (5.22 mg, 17.7% yield).

Compound 181 was prepared according to the procedure outlined for Compound 179 using 1-bromo-2-methoxyethane in Step 1 to provide 2⁵-((2-methoxyethyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (4.33 mg, 15.4% yield).

Compound 182 was prepared according to the procedure outlined for Compound 179 using 3-(bromomethyl)tetrahydrofuran in Step 1 to provide 1⁴-methyl-5⁵-(1H-pyrazol-1-yl)-2⁵-(((tetrahydrofuran-3-yl)methyl)sulfonyl)-4,6-diaza-5(2,6)-pyridina-1(1,4)-piperidina-2(1,2)-benzenacycloundecaphan-9-en-3-one (1.93 mg, 5.93% yield).

Compound 183 was prepared according to the procedure outlined for Compound 179 using 2-(bromomethyl)tetrahydrofuran in Step 1 to provide 1⁴-methyl-5⁵-(1H-pyrazol-1-yl)-2⁵-(((tetrahydrofuran-2-yl)methyl)sulfonyl)-4,6-diaza-5(2,6)-pyridina-1(1,4)-piperidina-2(1,2)-benzenacycloundecaphan-9-en-3-one (3.75 mg, 12.7% yield).

Compound 184 was prepared according to the procedure outlined for Compound 179 using 2-bromopropan-1-ol in Step 1 to provide 25-((1-hydroxypropan-2-yl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (3.29 mg, 11.7% yield).

Compound 185 was prepared according to the procedure outlined for Compound 179 using 1-(bromomethyl)cyclopropane-1-carbonitrile in Step 1 to provide 1-(((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)methyl)cyclopropane-1-carbonitrile (2.38 mg, 8.14% yield).

Compound 186 was prepared according to the procedure outlined for Compound 179 using 1-bromo-2-methylpropan-2-ol in step 1 to provide 2⁵-((2-hydroxy-2-methylpropyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (1.91 mg, 6.61% yield).

Compound 187 was prepared according to the procedure outlined for Compound 179 using 6-bromo-2-oxaspiro[3.3]heptane in step 1 to provide 2⁵-((2-oxaspiro[3.3]heptan-6-yl)sulfonyl)-14-methyl-5⁵-(1H-pyrazol-1-yl)-4,6-diaza-5(2,6)-pyridina-1(1,4)-piperidina-2(1,2)-benzenacycloundecaphan-9-en-3-one (5.30 mg, 17.6% yield).

Compound 188 was prepared according to the procedure outlined for Compound 179 using tert-butyl 3-bromopyrrolidine-1-carboxylate in step 1 to provide tert-butyl 3-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)pyrrolidine-1-carboxylate (3.37 mg, 10.0% yield).

Compound 189 was prepared according to the procedure outlined for Compound 179 using 3,6-dioxabicyclo[3.1.0]hexane in step 1 to provide 2⁵-((trans-4-hydroxytetrahydrofuran-3-yl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (1.82 mg, 6.15% yield).

Compound 190 was prepared according to the procedure outlined for Compound 179 using 3-bromocyclobutan-1-ol in step 1 to provide 25-((3-hydroxycyclobutyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (5.49 mg, 19.1% yield).

Compound 191 was prepared according to the procedure outlined for Compound 179 using 1-(2-bromoethyl)-1H-pyrazole in step 1 to provide 2⁵-((2-(1H-pyrazol-1-yl)ethyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (7.75 mg, 25.8% yield).

Compound 192 was prepared according to the procedure outlined for Compound 179 using 1-bromo-2-(methylsulfonyl)ethane in step 1 to provide 1⁴-methyl-2⁵-((2-(methylsulfonyl)ethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (3.74 mg, 12.2% yield).

Compound 193 was prepared according to the procedure outlined for Compound 179 using 2-(chloromethoxy)acetonitrile in step 1 to provide 2-(2-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)ethoxy)acetonitrile (1.00 mg, 3.40% yield).

Compound 194 was prepared according to the procedure outlined for Compound 179 using 3-(bromomethyl)-3-methyloxetane in step 1 to provide 1⁴-methyl-2⁵-(((3-methyloxetan-3-yl)methyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (5.17 mg, 17.5% yield).

Compound 195 was prepared according to the procedure outlined for Compound 179 using (R)-4-chloro-3-hydroxybutanenitrile in step 1 to provide (R)-3-hydroxy-4-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)butanenitrile (4.84 mg, 16.4% yield).

Compound 196 was prepared according to the procedure outlined for Compound 179 using 3-(bromomethyl)oxetane in step 1 to provide 1⁴-methyl-2⁵-((oxetan-3-ylmethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (4.06 mg, 14.1% yield).

Compound 197 was prepared according to the procedure outlined for Compound 179 using 1-bromopropan-2-ol in step 1 to provide 2⁵-((2-hydroxypropyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (8.20 mg, 29.1% yield).

Compound 198 was prepared according to the procedure outlined for Compound 179 using 3-bromopropanenitrile in step 1 to provide 3-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)propanenitrile (0.79 mg, 2.8% yield).

Compound 199 was prepared according to the procedure outlined for Compound 179 using 1-bromocyclobutane-1-carbonitrile in step 1 to provide 4-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)-2-methylenebutanenitrile (0.78 mg, 2.7% yield).

Compound 200 was prepared according to the procedure outlined for Compound 179 using 4-bromotetrahydro-2H-thiopyran in step 1 to provide 25-((1,1-dioxidotetrahydro-2H-thiopyran-4-yl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (3.15 mg, 9.88% yield).

Compound 201 was prepared according to the procedure outlined for Compound 179 using 3-bromothietane 1,1-dioxide in step 1 to provide 25-((1,1-dioxidothietan-3-yl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (0.90 mg, 3.0% yield).

Compound 202 was prepared according to the procedure outlined for Compound 179 179 using (2-methyloxiran-2-yl)methanol in step 1 to provide 25-((2,3-dihydroxy-2-methylpropyl)sulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (10.0 mg, 33.7% yield).

Compound 203 was prepared according to the procedure outlined for Compound 179 using 3-bromotetrahydrofuran in step 1 to provide 1⁴-methyl-5⁵-(1H-pyrazol-1-yl)-2⁵-((tetrahydrofuran-3-yl)sulfonyl)-4,6-diaza-5(2,6)-pyridina-1(1,4)-piperidina-2(1,2)-benzenacycloundecaphan-9-en-3-one (5.51 mg, 19.1% yield).

Compound 204 was prepared according to the procedure outlined for Compound 179 was synthesized following the procedure for Compound 179 using (3-fluorooxetan-3-yl)methyl 4-methylbenzenesulfonate in step 1 to provide (5.95 mg, 20.0% yield).

Compound 205 was prepared according to the procedure outlined for Compound 179 using 2-(bromomethyl)oxetane in step 1 to provide 1⁴-methyl-2⁵-((oxetan-2-ylmethyl)sulfonyl)-5⁵-(1H-pyrazol-1-yl)-4,6-diaza-5(2,6)-pyridina-1(1,4)-piperidina-2(1,2)-benzenacycloundecaphan-9-en-3-one (1.98 mg, 6.88% yield).

Compound 206 was prepared according to the procedure outlined for Compound 179 using 2-bromoacetamide in step 1 to provide 2-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)acetamide (4.49 mg, 16.0% yield).

Compound 207 was prepared according to General Procedure 2 using Compound 304 and 4-(1H-pyrazol-4-yl)morpholine to afford (E)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

### Compounds 208 and 209.

CuI (5 mg, 0.02 mmol) was added to the mixture of 4-(1H-pyrazol-4-yl)morpholine (80 mg, 0.52 mmol), K₃PO₄ (95 mg, 0.45 mmol), 5⁵-bromo-2⁵-(ethylsulfonyl)-1⁴-methyl-1′-oxa-4,6-diaza-5(2,6)-pyridina-1(1,4)-piperidina-2(1,2)-benzena-cycloundecaphan-8-en-3-one (80 mg, 0.15 mmol) and (1R,2R)—N¹,N²-dimethylcyclohexane-1,2-diamine (2 mg, 0.01 mmol) in DMSO (1 mL) under nitrogen atmosphere. The mixture was stirred at 100° C. for 16 hours. The mixture was purified by Prep-HPLC with following condition (Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient (B %): 45% B to 56% B in 9 min; Wave Length: 254 nm/220 nm nm; RT1 (min): 7.27; 8.13) to afford two products:

First eluting isomer (Compound 209); and (E)-2⁵-(ethylsulfonyl)-1⁴-methyl-5⁵-(4-morpholino-1H-pyrazol-1-yl)-11-oxa-4,6-diaza-5(2,6)-pyridina-1(1,4)-piperidina-2(1,2)-benzenacycloundecaphan-8-en-3-one (2.8 mg, 3.0% yield).

Second eluting isomer (Compound 208): (Z)-2⁵-(ethylsulfonyl)-1⁴-methyl-5⁵-(4-morpholino-1H-pyrazol-1-yl)-11-oxa-4,6-diaza-5(2,6)-pyridina-1(1,4)-piperidina-2(1,2)-benzenacycloundecaphan-8-en-3-one (1.8 mg, 2.0% yield).

### Compounds 210 and 211.

### Step 1: ter-butyl 4-(morpholinomethyl)-1H-pyrazole-1-carboxylate

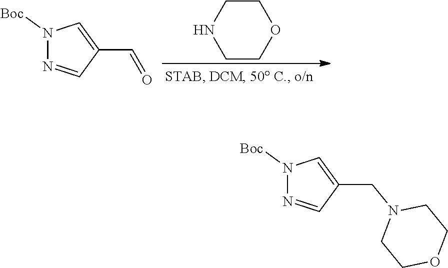 [SMILES: CC(C)(C)OC(=O)n1cc(C=O)cn1 ; C1COCCN1 ; CC(C)(C)OC(=O)n1cc(CN2CCOCC2)cn1]

STAB (2 g, 0.01 mol) was added into a mixture of morpholine (1 g, 0.01 mol) and tert-butyl 4-formyl-1H-pyrazole-1-carboxylate (2 g, 0.01 mol) in DCM (20 mL) at room temperature. The resulting mixture was stirred for overnight at 50° C. The resulting mixture was concentrated under vacuum. The reaction mixture was diluted with water (50 mL) and extracted with EA (3×50 mL). The combined organic layers were washed with brine (150 mL), dried over anhydrous Na₂SO₄, filtered and concentrated under vacuum to afford the crude product. The crude product was dissolved in DCM. Then the solution was uploaded to Flash Column Silica-CS (40 g) and eluting with THF in PE from 0% to 30% with 50 mL/min flow rate. Fractions with desired mass signal were combined and concentrated under vacuum to afford the desired product tert-butyl 4-(morpholinomethyl)-1H-pyrazole-1-carboxylate (200 mg, 7% yield). LCMS (ESI-MS) m/z=268.3 [M+H]⁺.

### Step 2: 4-((1H-pyrazol-4-yl)methyl)morpholine

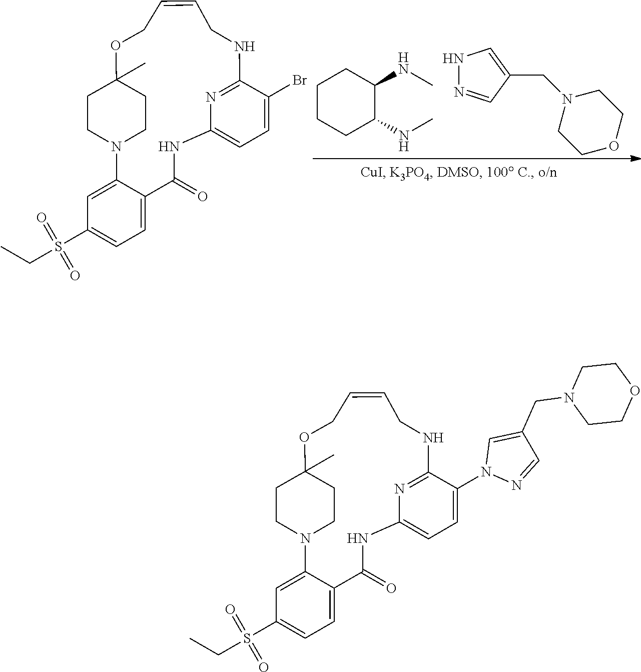

TFA (1 mL, 10 mmol) was added to tert-butyl 4-(morpholinomethyl)-1H-pyrazole-1-carboxylate (200 mg, 0.75 mmol) in DCM (4 mL). The mixture was stirred at room temperature for 1 hour. The resulting mixture was concentrated under vacuum. The residue was dissolved in aq. NaHCO₃ (5 mL). The reaction mixture was extracted with EA (3×5 mL). The combined organic layers were washed with brine (15 mL), dried over anhydrous Na₂SO₄, filtered and concentrated under vacuum to afford the crude product 4-((1H-pyrazol-4-yl)methyl)morpholine (100 mg, crude). LCMS (ESI-MS) m/z=168.2 [M+H]⁺.

### Step 3: 2⁵-(ethylsulfonyl)-14-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one

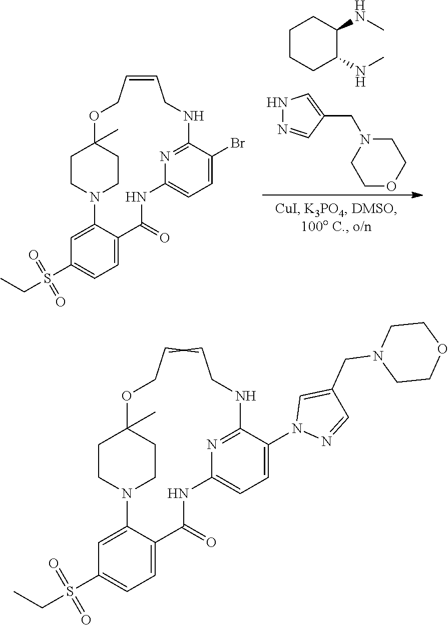

CuI (3.1 mg, 0.02 mmol) and (1R,2R)—N¹,N²-dimethylcyclohexane-1,2-diamine (2.3 mg, 0.02 mmol) were added to the mixture of 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (90 mg, 0.16 mmol), 4-((1H-pyrazol-4-yl)methyl)morpholine (27 mg, 0.16 mmol) and K₃PO₄ (68 mg, 0.49 mmol) in DMSO (1 mL) at room temperature under nitrogen atmosphere. The resulting mixture was stirred for overnight at 100° C. under nitrogen atmosphere. The residue was purified by reversed-phase flash chromatography with the following conditions: column, C18 silica gel; mobile phase, H₂O in ACN, 10% to 50% gradient in 10 min; detector, UV 254 nm, to afford the crude product 2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one (27 mg, crude). LCMS (ESI-MS) m/z=636.3 [M+H]⁺.

### Step 4: Compounds 210 and 211

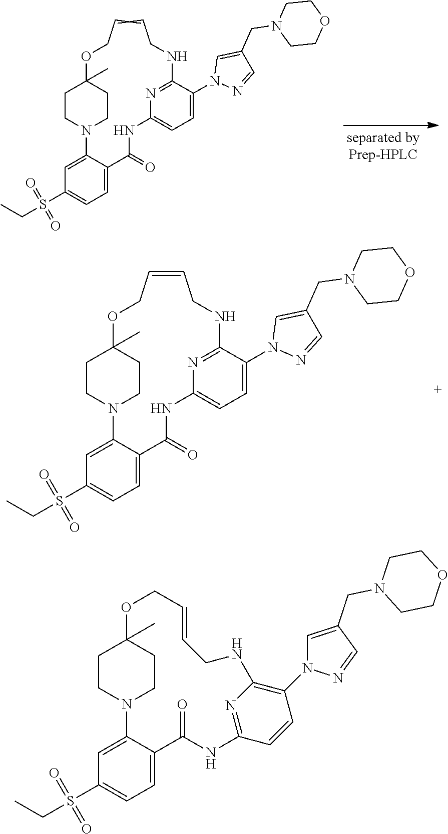 [SMILES: CCS(=O)(=O)c1ccc2c(c1)N1CCC(C)(CC1)OCC=CCNc1nc(ccc1-n1cc(CN3CCOCC3)cn1)NC2=O ; CCS(=O)(=O)c1ccc2c(c1)N1CCC(C)(CC1)OC/C=C\CNc1nc(ccc1-n1cc(CN3CCOCC3)cn1)NC2=O ; CCS(=O)(=O)c1ccc2c(c1)N1CCC(C)(CC1)OC/C=C/CNc1nc(ccc1-n1cc(CN3CCOCC3)cn1)NC2=O]

The crude product 2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one (27 mg, 0.04 mmol) was purified by Prep-Chiral-HPLC with the following conditions: Column: Xbridge Prep OBD Shield RP18, 30*150 mm, 5 μm; Mobile Phase A: Water (10 mmol/L NH₄HCO₃), Mobile Phase B: ACN; Flow rate: 60 mL/min; Wave Length: 254 nm/220 nm nm; RT1 (min): 6.0; 6.7 the desired fractions were combined and lyophilized to afford the two products: (E)-2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one (1.6 mg, 12% yield) and (Z)-2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one (3.5 mg, 24% yield).

Second eluting isomer (Compound 210): (Z)-2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one.

First eluting isomer (Compound 211): (E)-2⁵-(ethylsulfonyl)-1⁴-methyl-5⁵-(4-(morpholinomethyl)-1H-pyrazol-1-yl)-11-oxa-4,6-diaza-5(2,6)-pyridina-1(1,4)-piperidina-2(1,2)-benzenacycloundecaphan-8-en-3-one.

Compound 212 was prepared according to the procedure used to prepare Compound 179 using 1 2-bromopropanenitrile in Step 1 to provide (E)-2-((1⁴-methyl-3-oxo-5⁵-(1H-pyrazol-1-yl)-4,6-diaza-5(2,6)-pyridina-1(1,4)-piperidina-2(1,2)-benzenacycloundecaphan-9-en-2⁵-yl)sulfonyl)propanenitrile (0.43 mg, 2.0%).

### Compounds 213 and 214.

### Step 1: tert-butyl 3-(1-(6-amino-2-chloropyridin-3-yl)-1H-pyrazol-4-yl) azetidine-1-carboxylate

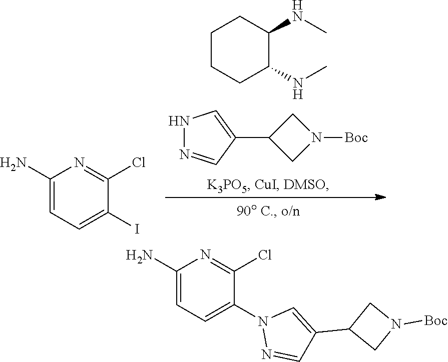

K₃PO₄ (3 g, 10 mmol) was added to the mixture of 6-chloro-5-iodopyridin-2-amine (1 g, 5 mmol), tert-butyl 3-(1H-pyrazol-4-yl) azetidine-1-carboxylate (1 g, 4 mmol), CuI (0.09 g, 0.1 Eq, 0.4 mmol) and (1R,2R)—N¹, N²-dimethylcyclohexane-1,2-diamine (0.06 g, 0.4 mmol) in DMSO (20 mL) under nitrogen atmosphere. The mixture was stirred at 90° C. overnight. The reaction mixture was purified by reversed-phase flash chromatography with the following conditions: column, C18 silica gel; mobile phase, aq. 0.04% TFA in ACN, 10% to 60% gradient in 10 min; detector, UV 254 nm to afford the product tert-butyl 3-(1-(6-amino-2-chloropyridin-3-yl)-1H-pyrazol-4-yl) azetidine-1-carboxylate (400 mg, 20% yield). LCMS (ESI-MS) m/z=350.1 [M+H]⁺.

### Step 2: tert-butyl 3-(1-(2-(allylamino)-6-aminopyridin-3-yl)-1H-pyrazol-4-yl) azetidine-1-carboxylate

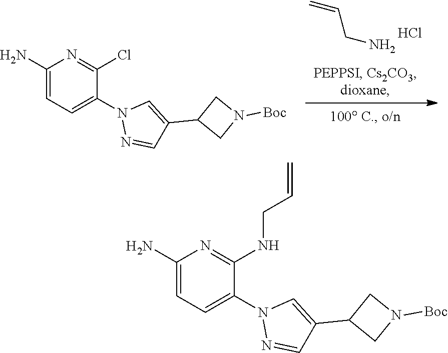

(SP-4-1)-[1,3-Bis[2,6-bis(1-propylbutyl)phenyl]-4,5-dichloro-1,3-dihydro-2H-imidazol-2-ylidene]dichloro-(3-chloropyridine-kappaN)-Palladium (111 mg, 0.11 mmol) was added to the mixture of prop-2-en-1-amine hydrochloride (321 mg, 3.43 mmol), tert-butyl 3-(1-(6-amino-2-chloropyridin-3-yl)-1H-pyrazol-4-yl)azetidine-1-carboxylate (400 mg, 1.14 mmol) and Cs₂CO₃ (1.12 g, 3.43 mmol) in dioxane (4 mL) under nitrogen atmosphere. The mixture was stirred at 100° C. overnight. The resulting mixture was filtered and the filter cake was washed with DCM (3×10 mL). The filtrate was concentrated under reduced pressure. The residue was purified by silica gel column chromatography, eluted with EA:PE (1:1) to afford tert-butyl 3-(1-(2-(allylamino)-6-aminopyridin-3-yl)-1H-pyrazol-4-yl) azetidine-1-carboxylate (360 mg, 72% yield). LCMS (ESI-MS) m/z=371.2 [M+H]⁺.

### Step 3: tert-butyl 3-(1-(2-(allylamino)-6-(2-(4-(allyloxy)-4-methylpiperidin-1-yl)-4-(ethylsulfonyl)-benza-mido) pyridin-3-yl)-1H-pyrazol-4-yl) azetidine-1-carboxylate

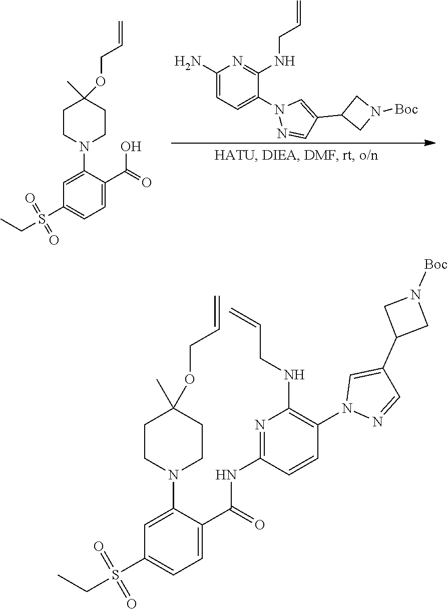 [SMILES: C=CCOC1(C)CCN(c2cc(S(=O)(=O)CC)ccc2C(=O)O)CC1 ; C=CCNc1nc(N)ccc1-n1cc(C2CN(C(=O)OC(C)(C)C)C2)cn1 ; C=CCNc1nc(NC(=O)c2ccc(S(=O)(=O)CC)cc2N2CCC(C)(OCC=C)CC2)ccc1-n1cc(C2CN(C(=O)OC(C)(C)C)C2)cn1]

DIEA (419 mg, 3.24 mmol) was added to the mixture of 2-(4-(allyloxy)-4-methylpiperidin-1-yl)-4-(ethylsulfonyl) benzoic acid (397 mg, 1.08 mmol), tert-butyl 3-(1-(2-(allylamino)-6-aminopyridin-3-yl)-1H-pyrazol-4-yl) azetidine-1-carboxylate (400 mg, 1.08 mmol) and HATU (616 mg, 1.62 mmol) in DMF (10 mL). The mixture was stirred at room temperature overnight. The reaction was poured into water (50 mL) at room temperature. The resulting mixture was extracted with EA (3×50 mL). The combined organic layers were washed with brine (150 mL), dried over anhydrous Na₂SO₄. After filtration, the filtrate was concentrated under reduced pressure. The crude product was purified by silica gel column chromatography, eluted with EA:PE (1:5) to afford tert-butyl 3-(1-(2-(allylamino)-6-(2-(4-(allyloxy)-4-methylpiperidin-1-yl)-4-(ethylsulfonyl)-benzamido) pyridin-3-yl)-1H-pyrazol-4-yl) azetidine-1-carboxylate (550 mg, 70.8% yield). LCMS (ESI-MS) m/z=720.3 [M+H]⁺.

### Step 4: tert-butyl 3-(1-(2⁵-(ethylsulfonyl)-14-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl) azetidine-1-carboxylate

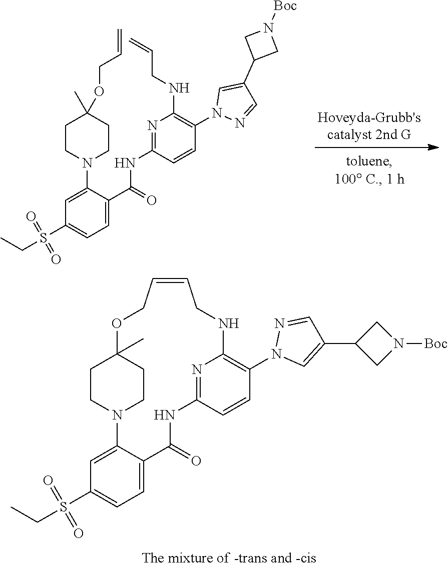 [SMILES: C=CCNc1nc(NC(=O)c2ccc(S(=O)(=O)CC)cc2N2CCC(C)(OCC=C)CC2)ccc1-n1cc(C2CN(C(=O)OC(C)(C)C)C2)cn1 ; CCS(=O)(=O)c1ccc2c(c1)N1CCC(C)(CC1)OC/C=C\CNc1nc(ccc1-n1cc(C3CN(C(=O)OC(C)(C)C)C3)cn1)NC2=O]

Hoveyda-Grubb's catalyst 2nd Generation (47.9 mg, 0.08 mmol) was added to tert-butyl 3-(1-(2-(allylamino)-6-(2-(4-(allyloxy)-4-methylpiperidin-1-yl)-4-(ethylsulfonyl) benzamido) pyridin-3-yl)-1H-pyrazol-4-yl) azetidine-1-carboxylate (550 mg, 0.76 mmol) in toluene (10 mL) under nitrogen atmosphere. The resulting mixture was concentrated under vacuum. The reaction mixture was purified by Preparative TLC (EA:PE=1:2) to afford the crude product tert-butyl 3-(1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)azetidine-1-carboxylate (60 mg, 11% yield). LCMS (ESI-MS) m/z=692.3 [M+H]⁺.

### Step 5: 5⁵-(4-(azetidin-3-yl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

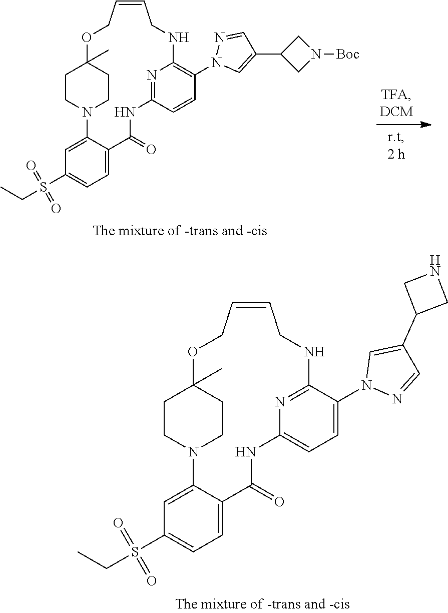 [SMILES: CCS(=O)(=O)c1ccc2c(c1)N1CCC(C)(CC1)OC/C=C\CNc1nc(ccc1-n1cc(C3CN(C(=O)OC(C)(C)C)C3)cn1)NC2=O ; CCS(=O)(=O)c1ccc2c(c1)N1CCC(C)(CC1)OC/C=C\CNc1nc(ccc1-n1cc(C3CNC3)cn1)NC2=O]

TFA (0.5 mL) was added to tert-butyl 3-(1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)azetidine-1-carboxylate (30 mg, 0.04 mmol) in DCM (1 mL). The mixture was stirred at 25° C. for 1 hour. The reaction mixture was concentrated under vacuum to afford the crude product 5⁵-(4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (10 mg crude). LCMS (ESI-MS) m/z=592.3 [M+H]⁺.

Step 6: Compounds 214 and 215: 5⁵-(4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (10 mg crude) was purified by Prep-HPLC with following condition (Column: X-Bridge Prep OBD C18 Column, 19*250 mm, 5 μm; Mobile Phase A: Water(10 mmol/L NH₄HCO₃), Mobile Phase B: ACN; Flow rate: 25 mL/min mL/min; Gradient (B %): 44% B to 61% B in 10 min; Wave Length: 254 nm/220 nm nm; RT1 (min): 8.45; 9.23) to afford two products:

First eluting isomer (Compound 214): (E)-5⁵-(4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (6 mg, 20% yield).

Second eluting isomer (Compound 213): (Z)-5⁵-(4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (10 mg, 37% yield).

### Compounds 215 and 216.

### Step 1: tert-butyl 3-((1H-pyrazol-4-yl)amino)azetidine-1-carboxylate

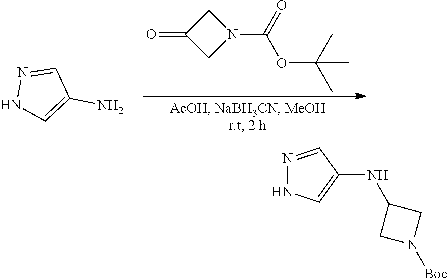 [SMILES: Nc1cn[nH]c1 ; CC(C)(C)OC(=O)N1CC(=O)C1 ; CC(C)(C)OC(=O)N1CC(Nc2cn[nH]c2)C1]

To a stirred solution of 1H-pyrazol-4-amine (500 mg, 6.02 mmol) in anhydrous MeOH (10 mL) was added tert-butyl 3-oxoazetidine-1-carboxylate (1.03 g, 6.02 mmol) and AcOH (181 mg, 3.01 mmol) at room temperature 0.5 hour, The added Sodium cyanoborohydride (567 mg, 9.03 mmol). The mixture was stirred at room temperature for 2 hours. The reaction mixture was purified by C.C. with following condition (ACN in aq.0.04% NH₄HCO₃, 20%) to afford the product tert-butyl 3-((1H-pyrazol-4-yl)amino)azetidine-1-carboxylate (1.06 g, 59% yield). LCMS (ESI-MS) m/z=182.9 [M-tbu]⁺.

### Step 2: tert-butyl (Z)-3-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)amino)azetidine-1-carboxylate

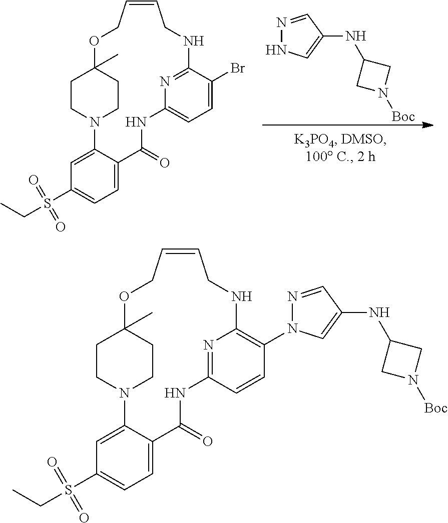 [SMILES: CCS(=O)(=O)c1ccc2c(c1)N1CCC(C)(CC1)OC/C=C\CNc1nc(ccc1Br)NC2=O ; CC(C)(C)OC(=O)N1CC(Nc2cn[nH]c2)C1 ; CCS(=O)(=O)c1ccc2c(c1)N1CCC(C)(CC1)OC/C=C\CNc1nc(ccc1-n1cc(NC3CN(C(=O)OC(C)(C)C)C3)cn1)NC2=O]

To a solution of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (90 mg, 0.16 mmol), tert-butyl 3-((1H-pyrazol-4-yl)amino)azetidine-1-carboxylate (39 mg, 0.16 mmol) and K₂CO₃ (23 mg, 0.16 mmol) in DMSO (1 mL) were added (1R)—N¹,N²-dimethylcyclohexane-1,2-diamine (23 mg, 0.16 mmol) and CuI (31 mg, 0.16 mmol). After stirring for 2 hours at 100° C. under nitrogen atmosphere. After cooling to room temperature, the reaction mixture was diluted with H₂O (3 mL) and extracted with EA (3×3 mL). The combined organic layers were washed with brine (10 mL), dried over anhydrous Na₂SO₄, filtered and concentrated under vacuum to afford the crude product. The residue was purified by silica gel column chromatography, eluted with PE:EA (1:1) to afford tert-butyl (Z)-3-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)amino)azetidine-1-carboxylate (120 mg, 81% yield). LCMS (ESI-MS) m/z=707.3 [M+H]⁺.

Step 3: Compounds 215 and 216: Into a 25 mL round-bottom flask were added tert-butyl (Z)-3-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)amino)azetidine-1-carboxylate (50 mg, 0.07 mmol), DCM (3.6 mL) and TFA (0.7 mL, 9.0 mmol) at room temperature. The resulting mixture was stirred for 25° C. at 2 hours. The resulting mixture was concentrated under reduced pressure. The crude product (130 mg) was purified by Prep-HPLC with the conditions (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient (B %): 29% B to 41% B in13 min; Wave Length: 254 nm/220 nm nm; RT1 (min): 6.95 7.65) to afford:

First eluting isomer (Compound 215): (Z)-5⁵-(4-(azetidin-3-ylamino)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (0.5 mg, 1% yield).

Second eluting isomer (Compound 216): (E)-5⁵-(4-(azetidin-3-ylamino)-1H-pyrazol-1-yl)-25-(ethylsulfonyl)-1⁴-methyl-11-oxa-4,6-diaza-5(2,6)-pyridina-1(1,4)-piperidina-2(1,2)-benzenacycloundecaphan-8-en-3-one (1.89 mg, 2.8% yield).

Compound 217 was prepared according to the procedure outlined for Compound 179 using 1-chlorobut-2-yne in step 1 to provide (E)-2⁵-(but-2-yn-1-ylsulfonyl)-1⁴-methyl-5⁵-(1H-pyrazol-1-yl)-4,6-diaza-5(2,6)-pyridina-1(1,4)-piperidina-2(1,2)-benzenacycloundecaphan-9-en-3-one (1.1 mg, 4% yield).

Compound 218 was prepared according to General Procedure 2 using Compound 300 and 4-(oxolan-3-yl)-1H-pyrazole to afford (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one (4.1 mg, 15.6% yield).

Compound 219 was prepared according to General Procedure 2 using Compound 300 and 1-methyl-4-(1H-pyrazol-4-yl)piperidine to afford (E)-2⁵-(ethylsulfonyl)-55-(4-(1-methylpiperidin-4-yl)-1H-pyrazol-1-yl)-4,6-diaza-5(2,6)-pyridina-1(1,4)-piperidina-2(1,2)-benzenacycloundecaphan-9-en-3-one (0.9 mg, 4.2% yield).

Compound 220 was prepared according to General Procedure 2 using Compound 310 and 1-methyl-4-(1H-pyrazol-4-yl) to afford (Z)—N-(1⁴-methyl-5⁵-(4-(1-methylpiperidin-4-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (9.3 mg, 32.9% yield).

Compound 221 was prepared using Compound 310 and 4-(cyclopentyloxy)-1H-pyrazole via General Procedure 2 to afford (Z)—N-(5⁵-(4-(cyclopentyloxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (16.9 mg, 71.7% yield).

### Compounds 222 and 224.

### Step 1: tert-butyl 4-cyclopropoxy-1H-pyrazole-1-carboxylate

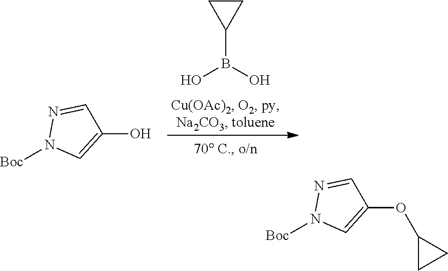 [SMILES: OB(O)C1CC1 ; CC(C)(C)OC(=O)n1cc(O)cn1 ; CC(C)(C)OC(=O)n1cc(OC2CC2)cn1]

Cu(OAc)₂ (1 g, 8 mmol) was added to the mixture of tert-butyl 4-hydroxy-1H-pyrazole-1-carboxylate (1 g, 5 mmol), Na₂CO₃ (623 mg, 5.54 mmol), pyridine (0.6 g, 8 mmol) and cyclopropylboronic acid (1 g, 0.01 mol) in toluene (20 mL) under oxygen atmosphere. The mixture was stirred at 70° C. overnight. The resulting mixture was concentrated under vacuum. The residue was diluted with water (50 mL). The resulting mixture was extracted with EA (3×50 mL). The combined organic layers were washed with brine (100 mL), dried over anhydrous Na₂SO₄. After filtration, the filtrate was concentrated under reduced pressure. The residue was purified by silica gel column chromatography, eluted with THF/PE (1:9) to afford tert-butyl 4-cyclopropoxy-1H-pyrazole-1-carboxylate (200 mg, 20% yield).

### Step 2: 4-cyclopropoxy-1H-pyrazole

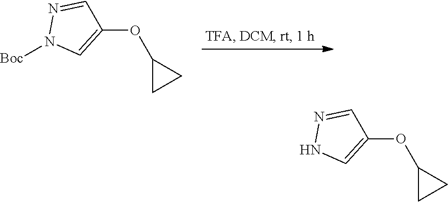 [SMILES: CC(C)(C)OC(=O)n1cc(OC2CC2)cn1 ; c1n[nH]cc1OC1CC1]

TFA (0.5 mL, 6 mmol) was added to the mixture of tert-butyl 4-cyclopropoxy-1H-pyrazole-1-carboxylate (200 mg, 0.89 mmol) in DCM (3 mL). The mixture was stirred at room temperature for 1 hour. The resulting mixture was concentrated under vacuum to afford the crude product 4-cyclopropoxy-1H-pyrazole (120 mg, crude). LCMS (ESI-MS) m/z=125.1 [M+H]⁺.

### Step 3: Compounds 222 and 224

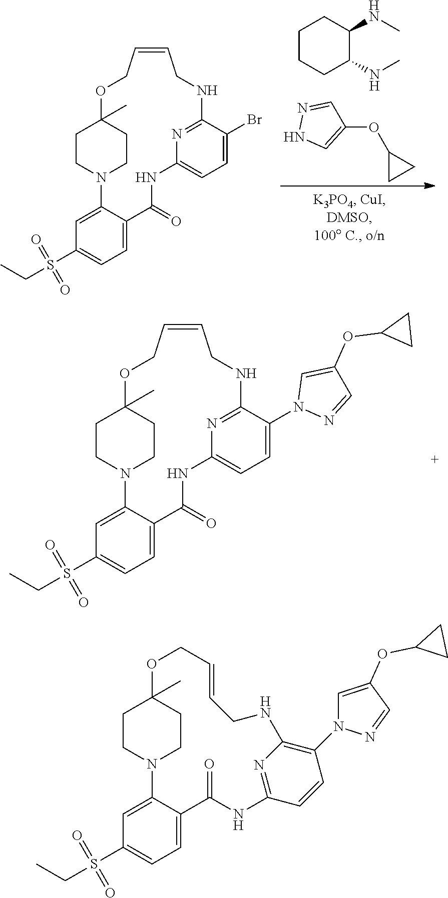

CuI (104 mg, 0.54 mmol) was added to the mixture of 5⁵-bromo-2⁵-(ethylsulfonyl)-1⁴-methyl-1¹-oxa-4,6-diaza-5(2,6)-pyridina-1(1,4)-piperidina-2(1,2)-benzenacycloundecaphan-8-en-3-one (200 mg, 0.36 mmol), (1R,2R)—N¹,N²-dimethylcyclohexane-1,2-diamine (51.8 mg, 0.36 mmol) and K₃PO₄ (232 mg, 1.09 mmol) in DMSO (5 mL) under nitrogen atmosphere. The reaction mixture was stirred at 100° C. overnight. The mixture was purified by Prep-HPLC with condition (Column: X-Bridge Prep OBD C18 Column, 30*150 mm, 5 μm; Mobile Phase A: Water(10 nmol/LNH₄HCO₃), Mobile Phase B: ACN; Flow rate: 60 mL/min mL/min; Gradient (B %): 45% B to 58% B in13 min; Wave Length: 254 nm/220 nm nm; RT1 (min): 7.47; 8.65) to afford the two products:

First eluting isomer (Compound 222): (E)-5⁵-(4-cyclopropoxy-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one.

Second eluting isomer (Compound 224): (Z)-5⁵-(4-cyclopropoxy-1H-pyrazol-1-yl)-2⁵-(ethylsulfonyl)-1⁴-methyl-1′-oxa-4,6-diaza-5(2,6)-pyridina-1(1,4)-piperidina-2(1,2)-benzenacycloundecaphan-8-en-3-one.

Compound 223 was prepared according to General Procedure 2 using Compound 302 and 4-cyclobutoxy-1H-pyrazole to afford (E)-5⁵-(4-cyclobutoxy-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one (6 mg, 26.5% yield).

### Compounds 225 and 226.

### Step 1: tert-butyl 3-((1-benzyl-1H-pyrazol-4-yl)oxy)azetidine-1-carboxylate

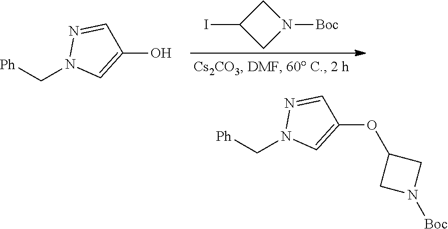 [SMILES: Oc1cnn(Cc2ccccc2)c1 ; CC(C)(C)OC(=O)N1CC(I)C1 ; CC(C)(C)OC(=O)N1CC(Oc2cnn(Cc3ccccc3)c2)C1]

1-benzyl-1H-pyrazol-4-ol (300 mg, 1.72 mmol) was added to a mixture of sodium tert-butyl 3-iodoazetidine-1-carboxylate (585 mg, 2.07 mmol) and Cs₂CO₃ (1.12 g, 3.44 mmol) in DMF (3 mL) at room temperature. The resulting mixture was stirred for 2 hours at 60° C. After cooling to room temperature, the reaction mixture was diluted with H₂O (5 mL) and extracted with EA (5×3 mL). The combined organic layers were washed with brine (15 mL), dried over anhydrous Na₂SO₄, filtered and concentrated under vacuum to afford the crude product. The residue was purified by silica gel column chromatography, eluted with PE:EA (1:1) to afford tert-butyl 3-((1-benzyl-1H-pyrazol-4-yl)oxy)azetidine-1-carboxylate (500 mg, 78% yield). LCMS (ESI-MS) m/z=330.0 [M+H]⁺.

### Step 2: tert-butyl 3-((1H-pyrazol-4-yl)oxy)azetidine-1-carboxylate

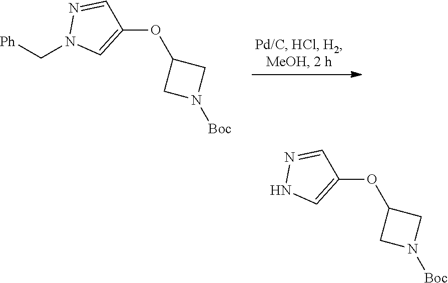 [SMILES: CC(C)(C)OC(=O)N1CC(Oc2cnn(Cc3ccccc3)c2)C1 ; CC(C)(C)OC(=O)N1CC(Oc2cn[nH]c2)C1]

To a solution of tert-butyl 3-((1-benzyl-1H-pyrazol-4-yl)oxy)azetidine-1-carboxylate (280 mg, 0.85 mmol) in MeOH (6 mL) was added Pd/C (10% on carbon, 10.0 mg) and 2 M HCl (0.03 mL) under nitrogen atmosphere in a 50 mL round-bottom flask. The mixture was hydrogenated at room temperature for 2 hours. The mixture was filtered, the filter cake was washed EA. The filtrate was concentrated under reduced pressure. The crude product was purified by reverse phase flash column choromatography with the following conditions (ACN/water 1:3) to afford tert-butyl 3-((1H-pyrazol-4-yl)oxy)azetidine-1-carboxylate (180 mg, 88.5% yield). LCMS (ESI-MS) m/z=184.0 [M-tBu]⁺.

### Step 3: tert-butyl (Z)-3-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)oxy)azetidine-1-carboxylate

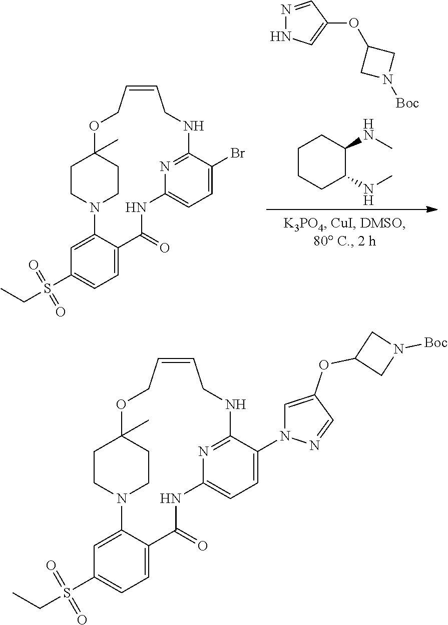

(1R,2R)—N¹,N²-dimethylcyclohexane-1,2-diamine (47 mg, 33 mmol) and CuI (78.0 mg, 0.41 mmol) were added to a mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (150 mg, 0.27 mmol), tert-butyl 3-((1H-pyrazol-4-yl)oxy)azetidine-1-carboxylate (200 mg, 0.84 mmol) and K₂CO₃ (76 mg, 0.55 mmol) in DMSO (1.5 mL) at room temperature under nitrogen atmosphere. The resulting mixture was stirred for 2 hours at 80° C. under nitrogen atmosphere. The resulting mixture was extracted with EA (3×5 mL). The combined organic layers were washed with H₂O (3×5 mL), dried over anhydrous Na₂SO₄. After filtration, the filtrate was concentrated under reduced pressure. The residue was purified by silica gel column chromatography, eluted with EA/PE (1:3) to afford tert-butyl (Z)-3-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)oxy)azetidine-1-carboxylate (50 mg, 26% yield). LCMS (ESI-MS) m/z=708.3 [M+H]⁺.

Step 4: Compounds 225 and 226: Into a 25 mL round-bottom flask were added tert-butyl (Z)-3-((1-(2⁵-(ethylsulfonyl)-14-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)oxy)azetidine-1-carboxylate (50 mg, 0.07 mmol), DCM (1 mL) and TFA (9 mg, 0.08 mmol) at room temperature. The resulting mixture was stirred for room temperature at 2 hours. The resulting mixture was concentrated under reduced pressure. The crude product (40 mg) was purified by Prep-HPLC with the conditions (Column: CHIRALPAK IC₃; Mobile Phase A: Hex (0.3% IPAmine): (MeOH:DCM=1:1)=10:90; Flow rate: 1 mL/min; Gradient (B %): isocratic; Injection Volume: 2 mL) to afford:

Second eluting isomer (Compound 225): (Z)-5⁵-(4-(azetidin-3-yloxy)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (2.7 mg, 6% yield).

First eluting isomer (Compound 226): (E)-5⁵-(4-(azetidin-3-yloxy)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (3.3 mg, 8% yield).

### Compounds 227 and 274.

### Step 1: 2-(1-(tetrahydro-2H-pyran-2-yl)-1H-pyrazol-4-yl) isothiazolidine 1,1-dioxide

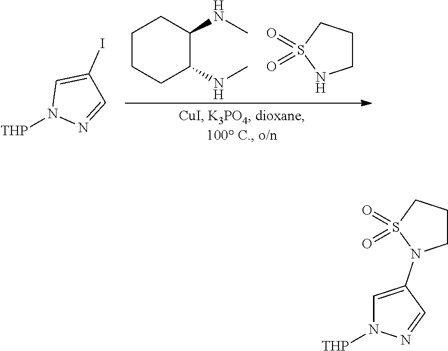

K₃PO₄ (1.14 g, 5.39 mmol) was added to the mixture of 4-iodo-1-(tetrahydro-2H-pyran-2-yl)-1H-pyrazole (500 mg, 1.80 mmol), isothiazolidine 1,1-dioxide (436 mg, 3.60 mmol), (1R,2R)—N¹, N²-dimethylcyclohexane-1,2-diamine (384 mg, 2.70 mmol) and CuI (514 mg, 2.70 mmol) in DMSO (10 mL) under nitrogen atmosphere. The mixture was stirred at 100° C. overnight. After cooling to room temperature, the reaction mixture was diluted with H₂O (100 mL) and extracted with EA (2×100 mL). The combined organic layers were washed with brine (200 mL), dried over anhydrous Na₂SO₄, filtered and concentrated under vacuum to afford the crude product. The crude product purified by silica gel column chromatography, eluted with PE:THF (5:1) to afford 2-(1-(tetrahydro-2H-pyran-2-yl)-1H-pyrazol-4-yl) isothiazolidine 1,1-dioxide (300 mg, 52% yield). LCMS (ESI-MS) m/z=272.1 [M+H]⁺.

### Step 2: 2-(1H-pyrazol-4-yl) isothiazolidine 1,1-dioxide

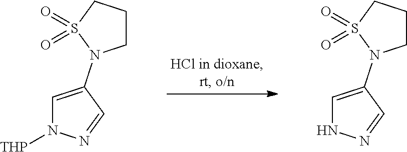

4 M HCl in dioxane (0.5 mL) was added to the mixture of 2-(1-(tetrahydro-2H-pyran-2-yl)-1H-pyrazol-4-yl) isothiazolidine 1,1-dioxide (300 mg, 1.11 mmol) in dioxane (5 mL) under nitrogen atmosphere. The mixture was stirred at room temperature overnight. The resulting mixture was concentrated under vacuum to afford the crude product 2-(1H-pyrazol-4-yl) isothiazolidine 1,1-dioxide (160 mg, crude). LCMS (ESI-MS) m/z=188.0 [M+H]⁺.

Step 3: Compounds 227 and 274: K₃PO₄ (116 mg, 0.55 mmol) was added to the mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (100 mg, 0.18 mmol), 2-(1H-pyrazol-4-yl)isothiazolidine 1,1-dioxide (68.1 mg, 0.36 mmol), (1R,2R)—N¹,N²-dimethylcyclohexane-1,2-diamine (38.8 mg, 0.27 mmol) in DMSO (5 mL) under nitrogen atmosphere. The mixture was stirred at 80° C. overnight. The mixture was purified by Prep-HPLC with following condition (Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient (B %): 40% B to 52% B in 10 min; Wave Length: 254 nm/220 nm nm; RT1 (min): 8.45; 9.52) to afford two products:

First eluting isomer (Compound 227): (Z)-5⁵-(4-(1,1-dioxidoisothiazolidin-2-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (8.0 mg, 6.0% yield).

Second eluting isomer (Compound 274): (E)-5⁵-(4-(1,1-dioxidoisothiazolidin-2-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacyclou-ndecaphan-8-en-3-one (3.7 mg, 3.0% yield).

### Compounds 228 and 265.

Step 1: tert-butyl 3-fluoro-4-morpholino-1H-pyrazole-1-carboxylate

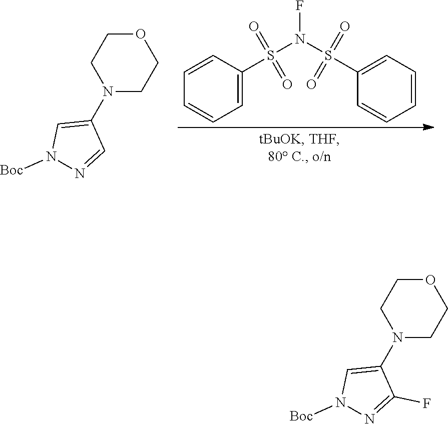

tBuONa (0.6 g, 6 mmol) was added to a stirred mixture of tert-butyl 4-morpholino-1H-pyrazole-1-carboxylate (1 g, 4 mmol) and N-fluoro-N-(phenylsulfonyl) benzenesulfonamide (2 g, 6 mmol) in THF (20 mL). The resulting mixture was stirred for 80° C. at 16 hours. After cooling to room temperature, the reaction mixture was diluted with H₂O (25 mL) and extracted with EA (2×25 mL). The combined organic layers were washed with brine (40 mL), dried over anhydrous Na₂SO₄, filtered and concentrated under vacuum to afford the crude product. The crude product purified by silica gel column chromatography, eluted with PE:EA (3:1) to afford tert-butyl 3-fluoro-4-morpholino-1H-pyrazole-1-carboxylate (110 mg, 10% yield). LCMS (ESI-MS) m/z=272.1 [M+H]⁺.

### Step 2: 4-(3-fluoro-1H-pyrazol-4-yl) morpholine

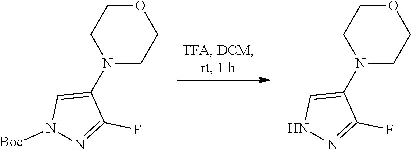 [SMILES: CC(C)(C)OC(=O)n1cc(N2CCOCC2)c(F)n1 ; Fc1n[nH]cc1N1CCOCC1]

TFA (1 mL) was added to a stirred mixture of tert-butyl 3-fluoro-4-morpholino-1H-pyrazole-1-carboxylate (110 mg, 0.4 mmol) in DCM (3 mL). The resulting mixture was stirred for 25° C. at 1 hour. The resulting mixture was concentrated under reduced pressure to afford 4-(3-fluoro-1H-pyrazol-4-yl) morpholine (130 mg, crude). LCMS (ESI-MS) m/z=172.1 [M+H]⁺.

Step 3: Compounds 228 and 265: tBuONa (49 mg, 0.51 mmol) was added to the mixture of 4-(3-fluoro-1H-pyrazol-4-yl)morpholine (65 mg, 0.38 mmol), 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (70 mg, 0.13 mmol), CuI (2.4 mg, 0.01 mmol) and N,N-Bis(2-phenylethyl)oxamide (3.8 mg, 0.01 mol) in DMSO (3 mL) under nitrogen atmosphere. The mixture was stirred at 100° C. overnight. The mixture was purified by Prep-HPLC with following condition (Column: X-Bridge Prep Shield RP18 OBD C18 Column, 30*150 mm, 5 μm; Mobile Phase A: Water(10 mmol/LNH₄HCO₃), Mobile Phase B: ACN; Flow rate: 60 mL/min mL/min; Gradient (B %): 44% B to74% B in 10 min; Wave Length: 254 nm/220 nm n; RT1 (min): 7.9; 8.6) to afford two products:

First eluting isomer (Compound 228) (Z)-2⁵-(ethylsulfonyl)-5⁵-(3-fluoro-4-morpholino-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (7.1 mg, 8% yield).

Second eluting isomer (Compound 265) (E)-2⁵-(ethylsulfonyl)-5⁵-(3-fluoro-4-morpholino-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (3.4 mg, 4% yield).

### Compound 229.

### Step 1: methyl 2-fluoro-4-((2-hydroxyethyl)thio)benzoate

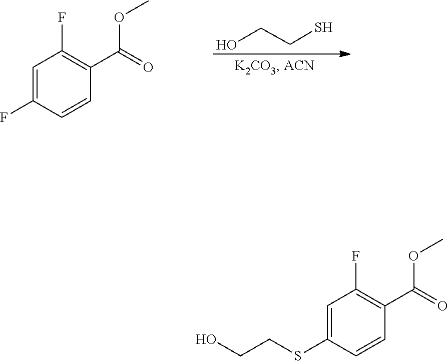

Methyl 2,4-difluorobenzoate (430 g, 2.50 mol, 1.5 equiv), 2-mercaptoethan-1-ol (130 g, 1.67 mol, 1 equiv), K₂CO₃ (345 g, 2.50 mol, 1.5 equiv) and ACN (4.3 L) were added into a flask at 25±5° C. The mixture was stirred for 4 h at 50° C. The reaction was filtered. The solution was concentrated under reduced pressure. methyl 2-fluoro-4-((2-hydroxyethyl)thio)benzoate (610 g, crude) was obtained. The crude product was used in the next step directly without further purification.

### Step 2: methyl 2-fluoro-4-((2-hydroxyethyl)sulfonyl)benzoate

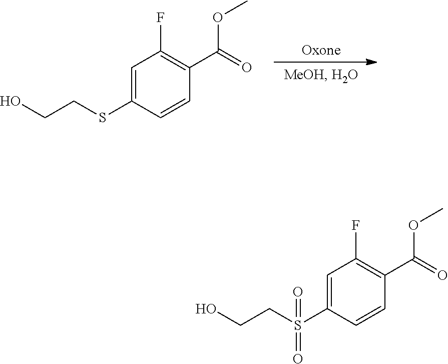 [SMILES: COC(=O)c1ccc(SCCO)cc1F ; COC(=O)c1ccc(S(=O)(=O)CCO)cc1F]

Methyl 2,4-difluorobenzoate (610 g, 2.65 mol, 1 equiv), MeOH (3.05 L) and H₂O (3.05 L) were added into a flask at 25±5° C. The mixture was cooled to 0° C. and oxone (2.75 kg, 7.96 mol, 3 equiv) was added into reactor at 0° C. in batches. After stirring for 16 h at 0° C., the reaction mixture was filtered. The filtrate was quenched by the addition of H₂O (10 V) at 25±5° C. and the mixture was extracted with EA (3×10 V). The combined organic layers were then dried over Na₂SO₄, and the solution was concentrated under reduced pressure. The crude product was further purified by stirring with heptane (10 V) for 2 h at room temperature. The precipitated solids were collected by filtration and were rinsed with heptane (2 V) to afford methyl 2-fluoro-4-((2-hydroxyethyl)sulfonyl)benzoate (172 g, 29% over two steps).

### Step 3: methyl 2-(4-(allyloxy)-4-methylpiperidin-1-yl)-4-((2-hydroxyethyl)sulfonyl)benzoate

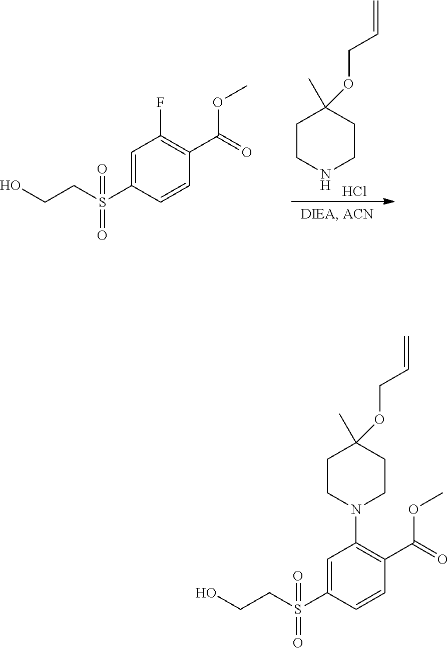 [SMILES: COC(=O)c1ccc(S(=O)(=O)CCO)cc1F ; C=CCOC1(C)CCNCC1 ; Cl ; C=CCOC1(C)CCN(c2cc(S(=O)(=O)CCO)ccc2C(=O)OC)CC1]

methyl 2-fluoro-4-((2-hydroxyethyl)sulfonyl)benzoate (65 g, 248 mmol, 1 equiv), 4-(allyloxy)-4-methylpiperidine hydrochloride (47.4 g, 248 mmol, 1 equiv), DIEA (96.0 g, 744 mmol, 3 equiv) and ACN (650 mL) were added into a flask at 25±5° C. The mixture was stirred overnight at 70° C. The mixture was concentrated in vacuum to 2-3 V, and then added to the mixture H₂O (10 V) at 25±5° C., extracted with EA (3×10 V). And the organic was then dried over Na₂SO₄. The solution was concentrated under reduced pressure. methyl 2-(4-(allyloxy)-4-methylpiperidin-1-yl)-4-((2-hydroxyethyl) sulfonyl)benzoate (91 g, crude) was obtained. The crude product was used in the next step directly without further purification.

### Step 4: N-[5-bromo-6-(prop-2-en-1-ylamino)pyridin-2-yl]-4-{2-[(tert-butyldimethylsilyl)oxy]ethanesulfonyl}-2-[4-methyl-4-(prop-2-en-1-yloxy) piperidin-1-yl]benzamide

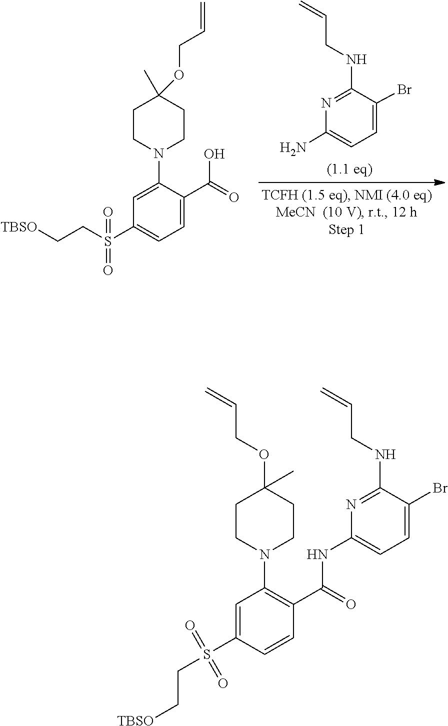 [SMILES: C=CCOC1(C)CCN(c2cc(S(=O)(=O)CCO[Si](C)(C)C(C)(C)C)ccc2C(=O)O)CC1 ; C=CCNc1nc(N)ccc1Br ; C=CCNc1nc(NC(=O)c2ccc(S(=O)(=O)CCO[Si](C)(C)C(C)(C)C)cc2N2CCC(C)(OCC=C)CC2)ccc1Br]

To a stirred solution of 4-{2-[(tert-butyldimethylsilyl)oxy]ethanesulfonyl}-2-[4-methyl-4-(prop-2-en-1-yloxy) piperidin-1-yl]benzoic acid (25 g, 50.2 mmol, 1 equiv), 1-methyl-1H-imidazole (16.5 g, 201 mmol, 4 equiv) and 3-bromo-N²-(prop-2-en-1-yl)pyridine-2,6-diamine (12.6 g, 55.2 mmol, 1.1 equiv) in MeCN (250 mL) was added TCFH (21.1 g, 75.3 mmol, 1.5 equiv) at 0° C. The resulting mixture was stirred at rt for 12 h. The mixture was quenched by the addition of water (500 mL). The resulting mixture was extracted with EtOAc (3×500 mL). The combined organic layers were dried over anhydrous Na₂SO₄. After filtration, the filtrate was concentrated under reduced pressure. The residue was purified by silica gel column chromatography, eluted with PE/EA (2:1) to afford N-[5-bromo-6-(prop-2-en-1-ylamino)pyridin-2-yl]-4-{2-[(tert-butyldimethylsilyl)oxy]ethanesulfonyl}-2-[4-methyl-4-(prop-2-en-1-yloxy) piperidin-1-yl]benzamide (31 g, 87% yield).

### Step 5: (E)-5⁵-bromo-2⁵-((2-((tert-butyldimethylsilyl)oxy)ethyl)sulfonyl)-1⁴-methyl-11-oxa-4,6-diaza-5(2,6)-pyridina-1(1,4)-piperidina-2(1,2)-benzenacycloundecaphan-8-en-3-one and (Z)-5⁵-bromo-2⁵-((2-((tert-butyldimethylsilyl)oxy)ethyl)sulfonyl)-1⁴-methyl-11-oxa-4,6-diaza-5(2,6)-pyridina-1(1,4)-piperidina-2(1,2)-benzenacycloundecaphan-8-en-3-one

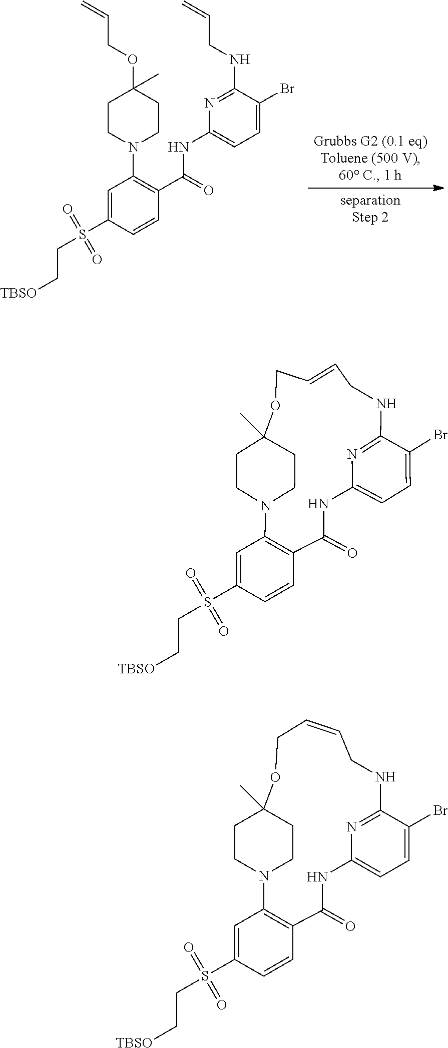 [SMILES: C=CCNc1nc(NC(=O)c2ccc(S(=O)(=O)CCO[Si](C)(C)C(C)(C)C)cc2N2CCC(C)(OCC=C)CC2)ccc1Br ; CC12CCN(CC1)c1cc(S(=O)(=O)CCO[Si](C)(C)C(C)(C)C)ccc1C(=O)Nc1ccc(Br)c(n1)NC/C=C/CO2 ; CC12CCN(CC1)c1cc(S(=O)(=O)CCO[Si](C)(C)C(C)(C)C)ccc1C(=O)Nc1ccc(Br)c(n1)NC/C=C\CO2]

A 20 L 3-necked round-bottom flask was charged with N-[5-bromo-6-(prop-2-en-1-ylamino)pyridin-2-yl]-4-{2-[(tert-butyldimethylsilyl)oxy]ethanesulfonyl}-2-[4-methyl-4-(prop-2-en-1-yloxy)piperidin-1-yl]benzamide (31 g, 43.8 mmol, 1 equiv) and toluene (15.5 L) at RT. Then, a solution of 2-[dichloro(phenylmethylidene) (tricyclohexyl-1\hat{ }[5]-phosphanyl)ruthenio]-1,3-bis(2,4,6-trimethylphenyl)imidazolidine (Grubbs Generation 2 catalyst, 3.7 g, 4.4 mmol, 0.1 equiv) in toluene (0.1 L) was added dropwise over 10 min while stirring at 50° C. under a nitrogen atmosphere. After stirring for 1 h at 60° C., the mixture was cooled and was concentrated under reduced pressure. The residue was purified by silica gel column chromatography, eluted with PE/EA (2:1) to afford mixture of E/Z isomer product (18 g, 60% yield). The residue was purified by reverse phase flash chromatography with the following conditions: column, C18 silica gel; mobile phase, MeCN in Water (10 mmol/L NH₄HCO₃), 95% to 95% gradient in 10 min; detector, UV 254 n. The resulting solution was concentrated under reduced pressure and dried by high vacuum. This resulted in (E)-5⁵-bromo-2⁵-((2-((tert-butyldimethylsilyl)oxy)ethyl)sulfonyl)-1⁴-methyl-11-oxa-4,6-diaza-5(2,6)-pyridina-1(1,4)-piperidina-2(1,2)-benzenacycloundecaphan-8-en-3-one (7.6703 g, 42.6% yield) and (Z)-5⁵-bromo-2⁵-((2-((tert-butyldimethylsilyl)oxy)ethyl)sulfonyl)-1⁴-methyl-11-oxa-4,6-diaza-5(2,6)-pyridina-1(1,4)-piperidina-2(1,2)-benzenacycloundecaphan-8-en-3-one (5.8620 g, 32.6% yield).

### Steps 6: (E)-5⁵-bromo-2⁵-((2-hydroxyethyl) sulfonyl)-1⁴-methyl-11-oxa-4,6-diaza-5(2,6)-pyridina-1(1,4)-piperidina-2(1,2)-benzenacycloundecaphan-8-en-3-one

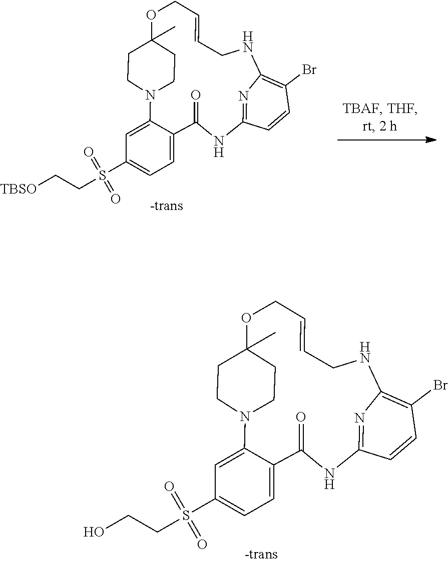 [SMILES: CC12CCN(CC1)c1cc(S(=O)(=O)CCO[Si](C)(C)C(C)(C)C)ccc1C(=O)Nc1ccc(Br)c(n1)NC/C=C/CO2 ; CC12CCN(CC1)c1cc(S(=O)(=O)CCO)ccc1C(=O)Nc1ccc(Br)c(n1)NC/C=C/CO2]

1M TBAF in THF (1.84 mL, 1.84 mmol) was added to the solution of (E)-5⁵-bromo-2⁵-((2-((tert-butyldimethylsilyl) oxy) ethyl) sulfonyl)-1⁴-methyl-11-oxa-4,6-diaza-5(2,6)-pyridina-1(1,4)-piperidina-2(1,2)-benzenacycloundecaphan-8-en-3-one (500 mg, 0.73 mmol) in THF (20 mL). The mixture was stirred at room temperature for 2 hours. The reaction was poured into aq. NH₄Cl (30 mL) at room temperature and the resulting mixture was extracted with EA (30 mL). The combined organic layers were washed with aq. NH4Cl (2×30 mL) and were dried over anhydrous Na₂SO₄. After filtration, the filtrate was concentrated under reduced pressure to afford the crude product (E)-5⁵-bromo-2⁵-((2-hydroxyethyl) sulfonyl)-1⁴-methyl-11-oxa-4,6-diaza-5(2,6)-pyridina-1(1,4)-piperidina-2(1,2)-benzenacycloundecaphan-8-en-3-one (375 mg, 90% yield). LCMS (ESI-MS) m/z=595.5 [M+H]⁺.

Steps 7 and 8: Compound 229: (E)-5⁵-bromo-2⁵-((2-hydroxyethyl) sulfonyl)-1⁴-methyl-11-oxa-4,6-diaza-5(2,6)-pyridina-1(1,4)-piperidina-2(1,2)-benzenacycloundecaphan-8-en-3-one was cross-coupled to 4-(1-((tert-butyldimethylsilyl)oxy)cyclopropyl)-1H-pyrazole, purified, and deprotected as described in Steps 1 and 2 in of the synthesis of Compounds 230, 271, 354, and 355 to afford (E)-2⁵-((2-hydroxyethyl)sulfonyl)-1⁴-methyl-5⁵-(4-propionyl-1H-pyrazol-1-yl)-11-oxa-4,6-diaza-5(2,6)-pyridina-1(1,4)-piperidina-2(1,2)-benzenacycloundecaphan-8-en-3-one (8 mg, 10% yield).

### Compounds 230, 271, 354, and 355.

### Step 1: 5⁵-(4-(1-((tert-butyldimethylsilyl) oxy) cyclopropyl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

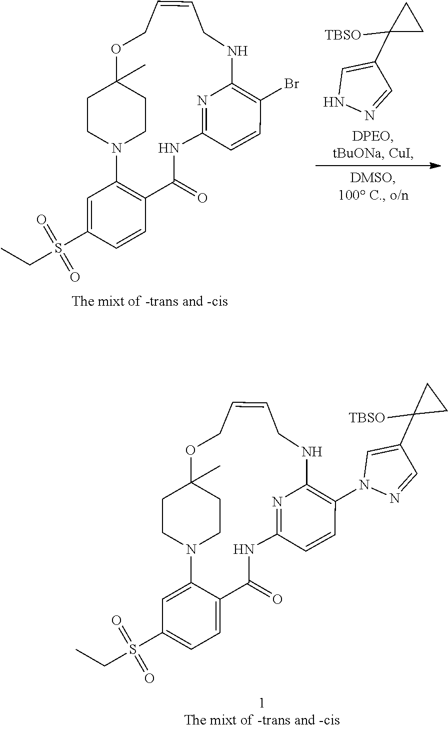 [SMILES: CCS(=O)(=O)c1ccc2c(c1)N1CCC(C)(CC1)OC/C=C\CNc1nc(ccc1Br)NC2=O ; CC(C)(C)[Si](C)(C)OC1(c2cn[nH]c2)CC1 ; CCS(=O)(=O)c1ccc2c(c1)N1CCC(C)(CC1)OC/C=C\CNc1nc(ccc1-n1cc(C3(O[Si](C)(C)C(C)(C)C)CC3)cn1)NC2=O]

CuI (0.20 equiv) was added to the mixture of 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (100 mg, 0.18 mmol), 4-(1-((tert-butyldimethylsilyl)oxy)cyclopropyl)-1H-pyrazole (1.0 equiv), t-BuONa (2.0 equiv) and N¹,N²-bis(furan-2-ylmethyl)oxalamide (0.20 equiv) in DMSO (15 V) under nitrogen atmosphere. The reaction was stirred at 80° C. for 16 hours. After cooling to room temperature, the reaction mixture was purified by reversed-phase flash chromatography with the following conditions: Column, C18 silica gel; mobile phase, aq. 0.04% TFA in ACN, 10% to 100% gradient in 10 min; detector, UV 254 nm to afford 5⁵-(4-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (40% yield). LCMS (ESI-MS) m/z=707.3 [M+H]⁺.

Step 2: Compounds 230, 271, 354, and 355:1 M TBAF in THF (2.0 equiv) was added to 5⁵-(4-(1-((tert-butyldimethylsilyl) oxy) cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (1.0 equiv) in THF (10 V). The mixture was stirred at room temperature for 1 hour. The reaction was poured into aq. NH₄Cl (50 V) at room temperature. The resulting mixture was extracted with EA (3×50 V). The combined organic layers were washed with aq. NH₄Cl (50 V), dried over anhydrous Na₂SO₄. After filtration, the filtrate was concentrated under reduced pressure. The crude product was purified by Prep-HPLC with following condition (Column: X-Bridge Prep OBD C18 Column, 30*150 mm, 5 μm; Mobile Phase A: Water(10 nmol/LNH₄HCO₃), Mobile Phase B: ACN; Flow rate: 60 mL/min mL/min; Gradient (B %): 34% B to 64% B in8 min; Wave Length: 254 nm/220 nm nm; RT1 (min): 7.07) to afford four peaks with desired product mass:

First eluting peak (Compound 271): (E)-2⁵-(ethylsulfonyl)-5⁵-(4-(1-hydroxycyclopropyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (6.9% yield).

Second eluting peak (Compound 230): (Z)-2⁵-(ethylsulfonyl)-5⁵-(4-(1-hydroxycyclopropyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (5.7% yield).

Third eluting peak (Compound 355): (E)-2⁵-(ethylsulfonyl)-1⁴-methyl-5⁵-(4-propionyl-1H-pyrazol-1-yl)-11-oxa-4,6-diaza-5(2,6)-pyridina-1(1,4)-piperidina-2(1,2)-benzenacycloundecaphan-8-en-3-one (1% yield).

Fourth eluting peak (Compound 354): (Z)-2⁵-(ethylsulfonyl)-1⁴-methyl-5⁵-(4-propionyl-1H-pyrazol-1-yl)-11-oxa-4,6-diaza-5(2,6)-pyridina-1(1,4)-piperidina-2(1,2)-benzenacycloundecaphan-8-en-3-one (2% yield).

Compound 231 was prepared according to General Procedure 2 using Compound 301 and [1-(1H-pyrazol-3-yl)cyclopentyl]methanol to afford (E)-2⁵-(ethylsulfonyl)-55-(3-(1-(hydroxymethyl)cyclopentyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (6.6 mg, 28.3% yield).

Compound 232 was prepared according to General Procedure 2 using Compound 301 and 2-([3-(trifluoromethyl)-1H-pyrazol-4-yl]oxy)ethan-1-ol to afford (E)-2⁵-(ethylsulfonyl)-5⁵-(4-(2-hydroxyethoxy)-3-(trifluoromethyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (3.4 mg, 13.7% yield).

Compound 233 was prepared according to General Procedure 2 using Compound 300 and 4-(oxolan-3-yloxy)-1H-pyrazole to afford (E)-2⁵-(ethylsulfonyl)-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-3-one (8.1 mg, 30.3% yield).

Compound 234 was prepared according to General Procedure 2 using Compound 300 and 4-(pyrrolidin-2-yl)-1H-pyrazole dihydrochloride to afford (E)-2⁵-(ethylsulfonyl)-5⁵-(4-(pyrrolidin-2-yl)-1H-pyrazol-1-yl)-4,6-diaza-5(2,6)-pyridina-1(1,4)-piperidina-2(1,2)-benzenacycloundecaphan-9-en-3-one (0.4 mg, 1.3% yield).

Compound 235 was prepared according to General Procedure 2 using Compound 300 and 4-cyclopropylmethoxy-1H-pyrazole to afford (E)-5⁵-(4-(cyclopropylmethoxy)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (4.6 mg, 21.1% yield).

Compound 236 was prepared according to General Procedure 2 using Compound 300 and 4-(cyclopentyloxy)-1H-pyrazole to afford (E)-5⁵-(4-(cyclopentyloxy)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (3.8 mg, 17% yield).

Compound 237 was prepared according to General Procedure 2 using Compound 300 and 2-methyl-2-(1H-pyrazol-3-yl)propan-1-ol to afford (E)-2⁵-(ethylsulfonyl)-5⁵-(3-(1-hydroxy-2-methylpropan-2-yl)-1H-pyrazol-1-yl)-4,6-diaza-5(2,6)-pyridina-1(1,4)-piperidina-2(1,2)-benzenacycloundecaphan-9-en-3-one (4 mg, 15.1% yield).

Compound 238 was prepared according to General Procedure 2 using Compound 300 and N,N-dimethyl-1H-pyrazole-4-carboxamide to afford (E)-1-(25-(ethylsulfonyl)-3-oxo-4,6-diaza-5(2,6)-pyridina-1(1,4)-piperidina-2(1,2)-benzenacycloundecaphan-9-en-5⁵-yl)-N,N-dimethyl-1H-pyrazole-4-carboxamide (0.5 mg, 2.3% yield).

Compound 239 was prepared according to General Procedure 2 using Compound 301 and and N-cyclopentyl-1H-pyrazol-4-amine to afford (E)-5⁵-(4-(cyclopentylamino)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (0.8 mg, 2% yield).

Compound 240 was prepared according to General Procedure 2 using Compound 300 and 4-(1H-pyrazol-4-yl)morpholin-3-one to afford (E)-4-(1-(25-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl)morpholin-3-one (0.65 mg, 2% yield).

Compound 241 was prepared according to General Procedure 2 using Compound 300 and [1-(1H-pyrazol-3-yl)cyclopentyl]methanol to afford (E)-2⁵-(ethylsulfonyl)-55-(3-(1-(hydroxymethyl)cyclopentyl)-1H-pyrazol-1-yl)-4,6-diaza-5(2,6)-pyridina-1(1,4)-piperidina-2(1,2)-benzenacycloundecaphan-9-en-3-one (2.4 mg, 10.5% yield).

Compound 242 was prepared according to General Procedure 2 using Compound 300 and 4-cyclobutoxy-1H-pyrazole to afford (E)-5⁵-(4-cyclobutoxy-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (6.8 mg, 26% yield).

Compound 243 was prepared according to General Procedure 2 using Compound 302 and N-[(1H-pyrazol-3-yl)methyl]acetamide to afford (Z)—N-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-3-yl)methyl)acetamide (1.2 mg, 4% yield).

Compound 244 was prepared according to General Procedure 2 using Compound 301 and N-[(1H-pyrazol-3-yl)methyl]acetamide to afford (E)-N-((1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-3-yl)methyl)acetamide (2.9 mg, 10.1% yield).

Compound 245 was prepared according to General Procedure 2 using Compound 301 and 2-(1H-pyrazol-4-yl)piperidine to afford (E)-2⁵-(ethylsulfonyl)-1⁴-methyl-5⁵-(4-(piperidin-2-yl)-1H-pyrazol-1-yl)-11-oxa-4,6-diaza-5(2,6)-pyridina-1(1,4)-piperidina-2(1,2)-benzenacycloundecaphan-8-en-3-one (1.4 mg, 4.8% yield).

Compound 246 was prepared according to General Procedure 2 using Compound 300 and N-[(1H-pyrazol-3-yl)methyl]acetamide to afford (E)-N-((1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-3-yl)methyl)acetamide (0.7 mg, 2.4% yield).

Compound 247 was prepared according to General Procedure 2 using Compound 302 and 2-(1H-pyrazol-4-yl)piperidine to afford (Z)-2⁵-(ethylsulfonyl)-1⁴-methyl-5⁵-(4-(piperidin-2-yl)-1H-pyrazol-1-yl)-11-oxa-4,6-diaza-5(2,6)-pyridina-1(1,4)-piperidina-2(1,2)-benzenacycloundecaphan-8-en-3-one (1.1 mg, 4.4% yield).

Compound 248 was prepared according to General Procedure 2 using Compound 300 and 2-(1H-pyrazol-4-yl)piperidine to afford (E)-2⁵-(ethylsulfonyl)-5⁵-(4-(piperidin-2-yl)-1H-pyrazol-1-yl)-4,6-diaza-5(2,6)-pyridina-1(1,4)-piperidina-2(1,2)-benzenacycloundecaphan-9-en-3-one (6.7 mg, 23.5% yield).

Compound 249 was prepared according to General Procedure 2 using Compound 300 and phenyl(1H-pyrazol-4-yl)methanol to afford (E)-2⁵-(ethylsulfonyl)-5⁵-(4-(hydroxy(phenyl)methyl)-1H-pyrazol-1-yl)-4,6-diaza-5(2,6)-pyridina-1(1,4)-piperidina-2(1,2)-benzenacycloundecaphan-9-en-3-one (1.8 mg, 7.9% yield).

Compound 250 was prepared according to General Procedure 2 using Compound 300 and 4-pyrrolidin-1-yl-1H-pyrazole to afford (E)-2⁵-(ethylsulfonyl)-5⁵-(4-(pyrrolidin-1-yl)-1H-pyrazol-1-yl)-4,6-diaza-5(2,6)-pyridina-1(1,4)-piperidina-2(1,2)-benzenacycloundecaphan-9-en-3-one (9.4 mg, 43% yield).

Compound 251 was prepared according to General Procedure 2 using Compound 301 and 4-cyclopropylmethoxy-1H-pyrazole to afford (E)-5⁵-(4-(cyclopropylmethoxy)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (8.9 mg, 39.4% yield).

Compound 252 was prepared according to General Procedure 2 using Compound 301 and 3-(methanesulfonylmethyl)-1H-pyrazole to afford (E)-2⁵-(ethylsulfonyl)-1⁴-methyl-5⁵-(3-((methylsulfonyl)methyl)-1H-pyrazol-1-yl)-11-oxa-4,6-diaza-5(2,6)-pyridina-1(1,4)-piperidina-2(1,2)-benzenacycloundecaphan-8-en-3-one (1.6 mg, 6.7% yield).

Compound 253 was prepared according to General Procedure 2 using Compound 300 and dimethyl[(1H-pyrazol-4-yl)methyl]amine dihydrochloride to afford (E)-5⁵-(4-((dimethylamino)methyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (1.4 mg, 4.9% yield).

Compound 254 was prepared according to General Procedure 2 using Compound 301 and dimethyl[(1H-pyrazol-4-yl)methyl]amine dihydrochloride to afford (E)-5⁵-(4-((dimethylamino)methyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (4.2 mg, 14.8% yield).

Compound 255 was prepared according to General Procedure 2 using Compound 300 and 3-cyclopropyl-1H-pyrazole to afford (E)-5⁵-(3-cyclopropyl-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (6.8 mg, 30.1% yield).

Compound 256 was prepared according to General Procedure 2 using Compound 300 and 1-(1H-pyrazol-4-yl)pyrrolidin-2-one to afford (E)-2⁵-(ethylsulfonyl)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (0.6 mg, 2.7% yield).

Compound 257 was prepared according to General Procedure 2 using Compound 300 and N-cyclopentyl-1H-pyrazol-4-amine to afford (E)-5⁵-(4-(cyclopentylamino)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (5.2 mg, 18.2% yield).

Compound 258 was prepared according to General Procedure 2 using Compound 301 and 4-(oxolan-3-yl)-1H-pyrazole to afford (E)-2⁵-(ethylsulfonyl)-1⁴-methyl-5⁵-(4-(tetrahydrofuran-3-yl)-1H-pyrazol-1-yl)-11-oxa-4,6-diaza-5(2,6)-pyridina-1(1,4)-piperidina-2(1,2)-benzenacycloundecaphan-8-en-3-one (6.3 mg, 28% yield).

Compound 259 was prepared according to General Procedure 2 using Compound 300 and [1-(1H-pyrazol-3-yl)cyclobutyl]methanol to afford (E)-2⁵-(ethylsulfonyl)-5⁵-(3-(1-(hydroxymethyl)cyclobutyl)-1H-pyrazol-1-yl)-4,6-diaza-5(2,6)-pyridina-1(1,4)-piperidina-2(1,2)-benzenacycloundecaphan-9-en-3-one (2.4 mg, 10.9% yield).

### Compounds 260 and 261.

### Step 1: N-cyclopropyl-1-(tetrahydro-2H-pyran-2-yl)-1H-pyrazol-4-amine

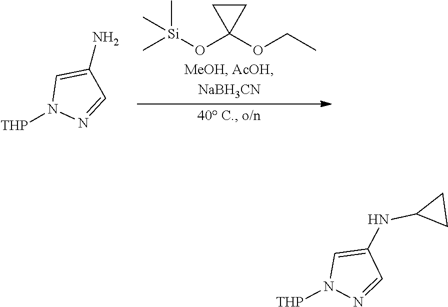

NaBH₃CN (2 g, 40 mmol) was added to the mixture of 1-(tetrahydro-2H-pyran-2-yl)-1H-pyrazol-4-amine (1 g, 6 mmol), (1-ethoxycyclopropoxy) trimethylsilane (2 g, 10 mmol) in MeOH (10 mL) and AcOH (1.0 mL). The mixture was stirred at 50° C. overnight. The mixture was poured into aq. NaHCO₃ (50 mL) at room temperature. The resulting mixture was extracted with EA (3×50 mL). The combined organic layers were washed with brine (150 mL), dried over anhydrous Na₂SO₄. After filtration, the filtrate was concentrated under reduced pressure. The residue was purified by silica gel column chromatography, eluted with THF:PE (1:2) to afford N-cyclopropyl-1-(tetrahydro-2H-pyran-2-yl)-1H-pyrazol-4-amine (280 mg, 20% yield). LCMS (ESI-MS) m/z=208.1 [M+H]⁺.

### Step 2: 4 cyclopropoxy-1H-pyrazole

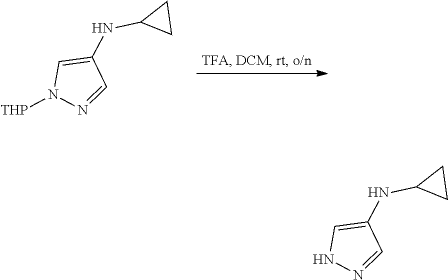

TFA (4 mL) was added to N-cyclopropyl-1-(tetrahydro-2H-pyran-2-yl)-1H-pyrazol-4-amine (400 mg, 1.93 mmol) in DCM (4 mL). The mixture was stirred at room temperature overnight. The resulting mixture was concentrated under vacuum. The residue was dissolved in aq. NaHCO₃ (5 mL). The resulting mixture was extracted with EA (3×5 mL). The combined organic layers were washed with brine (15 mL), dried over anhydrous Na₂SO₄. After filtration, the filtrate was concentrated under reduced pressure to afford the N-cyclopropyl-1H-pyrazol-4-amine (90 mg crude). LCMS (ESI-MS) m/z=124.1 [M+H]⁺.

Step 3: Compounds 260 and 261: K₂CO₃ (75.5 mg, 0.54 mmol) was added to the mixture of 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (100 mg, 0.18 mmol), N-cyclopropyl-1H-pyrazol-4-amine (22.4 mg, 0.18 mmol), (1R,2R)—N¹,N²-dimethylcyclohexane-1,2-diamine (38.8 mg, 0.27 mmol) and CuI (52.0 mg, 0.27 mmol) in DMSO (2 mL) under nitrogen atmosphere. The mixture was stirred at 100° C. overnight. The mixture was purified by Prep-HPLC with following condition (Column: Xselect CSH Prep Fluoro-Phenyl Column, 30*150 mm, 5 μm; Mobile Phase A: Water(0.1% FA), Mobile Phase B: ACN; Flow rate: 60 mL/min mL/min; Gradient (B %): 30% B to 60% B in 10 min; Wave Length: 254 nm/220 nm nm; RT1 (min): 8.1, 9.2 (min):) to afford two products:

First eluting isomer (Compound 260): (Z)-5⁵-(4-(cyclopropylamino)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (5.9 mg, 4.7% yield).

Second eluting isomer (261): (E)-5⁵-(4-(cyclopropylamino)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (2.2 mg, 1.7% yield).

### Compounds 262 and 263.

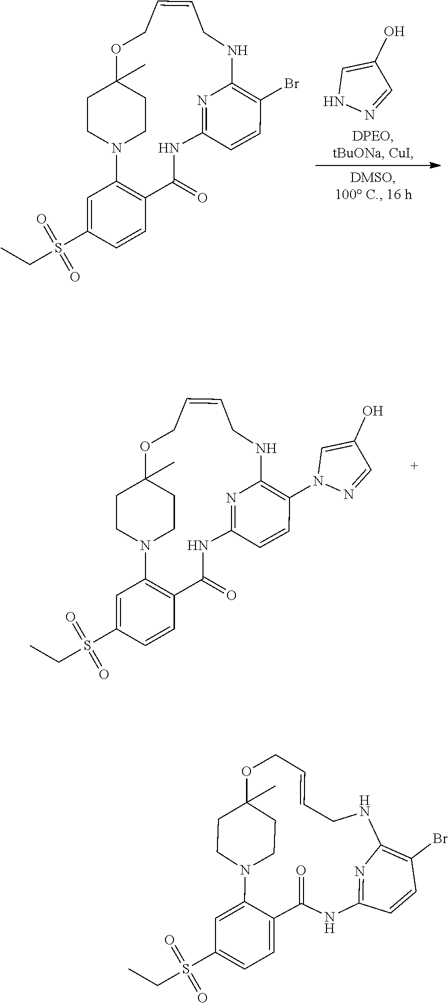 [SMILES: CCS(=O)(=O)c1ccc2c(c1)N1CCC(C)(CC1)OC/C=C\CNc1nc(ccc1Br)NC2=O ; Oc1cn[nH]c1 ; CCS(=O)(=O)c1ccc2c(c1)N1CCC(C)(CC1)OC/C=C\CNc1nc(ccc1-n1cc(O)cn1)NC2=O ; CCS(=O)(=O)c1ccc2c(c1)N1CCC(C)(CC1)OC/C=C/CNc1nc(ccc1Br)NC2=O]

t-BuONa (30 mg, 54 mmol) was added to the mixture of 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (100 mg, 0.18 mmol), 1H-pyrazol-4-ol (45.9 mg, 0.54 mmol), CuI (34.7 mg, 0.18 mmol) and N, N-bis(2-phenylethyl)ethanediamide (216 mg, 0.18 mmol) in DMSO (3 mL) under nitrogen atmosphere. The mixture was stirred at 100° C. for 16 hours. The mixture was purified by Prep-HPLC with following condition with Column: X-select CSH Prep Fluoro-Phenyl Column, 30*150 mm, 5 μm; Mobile Phase A: Water(0.1% FA), Mobile Phase B: ACN; Flow rate: 60 mL/min mL/min; Gradient (B %): 24% B to 54% B in 10 min; Wave Length: 254 nm/220 nm nm; RT1 (min): 7.5, 8.6 (min):, 9.3 (min) to afford two produce:

First eluting isomer (Compound 262): (Z)-2⁵-(ethylsulfonyl)-5⁵-(4-hydroxy-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (2.3 mg, 2.2% yield).

Second eluting isomer (Compound 263): (E)-2⁵-(ethylsulfonyl)-5⁵-(4-hydroxy-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (1.7 mg, 1.6% yield).

Compound 264 was prepared according to General Procedure 2 using Compound 311 and [1-(1H-pyrazol-3-yl)cyclobutyl]methanol to afford (E)-N-(55-(3-(1-(hydroxymethyl)cyclobutyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.8 mg, 28.8% yield).

Compound 266 was prepared according to General Procedure 2 using Compound 304 and 2-methyl-2-(1H-pyrazol-3-yl)propan-1-ol to afford (E)-N-(5⁵-(3-(1-hydroxy-2-methylpropan-2-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.3 mg, 5% yield).

Compound 267 was prepared according to General Procedure 2 using Compound 304 and 4-(cyclopentyloxy)-1H-pyrazole to afford (E)-N-(5⁵-(4-(cyclopentyloxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 8.9% yield).

Compound 268 was prepared according to General Procedure 2 using Compound 310 and 4-(oxolan-3-yloxy)-1H-pyrazole to afford (Z)—N-(1⁴-methyl-3-oxo-5⁵-(4-((tetrahydrofuran-3-yl)oxy)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (15.1 mg, 63.9% yield).

Compound 269 was prepared according to General Procedure 2 using Compound 310 and 1-(1H-pyrazol-3-yl)pyrrolidin-2-one to afford (Z)—N-(1⁴-methyl-3-oxo-5⁵-(3-(2-oxopyrrolidin-1-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (1.6 mg, 5.8% yield).

Compound 270 was prepared according to General Procedure 2 using Compound 304 and 4-cyclobutoxy-1H-pyrazole to afford (E)-N-(5⁵-(4-cyclobutoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.5 mg, 13.2% yield).

Compound 272 was prepared according to General Procedure 2 using Compound 311 and 4-cyclobutoxy-1H-pyrazole to afford (E)-N-(5⁵-(4-cyclobutoxy-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (9.2 mg, 40.2% yield).

Compound 273 was prepared according to General Procedure 2 using Compound 311 and 4-(oxolan-3-yloxy)-1H-pyrazole to afford (E)-N-(1⁴-methyl-3-oxo-5⁵-(4-((tetrahydrofuran-3-yl)oxy)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (6.9 mg, 29.2% yield).

Compound 275 was prepared according to General Procedure 2 using Compound 311 and 4-(1H-pyrrol-1-yl)-1H-pyrazole to afford (E)-N-(55-(4-(1H-pyrrol-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6 mg, 22.2% yield).

Compound 276 was prepared according to General Procedure 2 using Compound 304 and 1-(1H-pyrazol-3-yl)pyrrolidin-2-one to afford (E)-N-(3-oxo-5⁵-(3-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (1.9 mg, 7% yield).

Compound 277 was prepared according to General Procedure 2 using Compound 310 and 4-(1H-pyrrol-1-yl)-1H-pyrazole to afford (Z)—N-(5⁵-(4-(1H-pyrrol-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.4 mg, 23.5% yield).

Compound 278 was prepared according to General Procedure 2 using Compound 310 and 1-(1H-pyrazol-4-yl)pyrrolidin-2-one to afford (Z)—N-(1⁴-methyl-3-oxo-5⁵-(4-(2-oxopyrrolidin-1-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (8.5 mg, 30.9% yield).

Compound 279 was prepared according to General Procedure 2 using Compound 304 and 1-(1H-pyrazol-4-yl)pyrrolidin-2-one to afford (E)-N-(3-oxo-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (5.2 mg, 19.3% yield).

Compound 280 was prepared according to General Procedure 2 using Compound 304 and 4-pyrrolidin-1-yl-1H-pyrazole to afford (E)-N-(3-oxo-5⁵-(4-(pyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (8.6 mg, 32.2% yield).

Compound 281 was prepared according to General Procedure 2 using Compound 304 and 4-(oxolan-3-yloxy)-1H-pyrazole to afford E)-N-(3-oxo-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (1.3 mg, 4.7% yield).

### Compound 282.

### Step 1: ((E)-N-(5⁵-(4-(1-((tert-butyldimethylsilyl) oxy) cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl) ethanesulfonamide

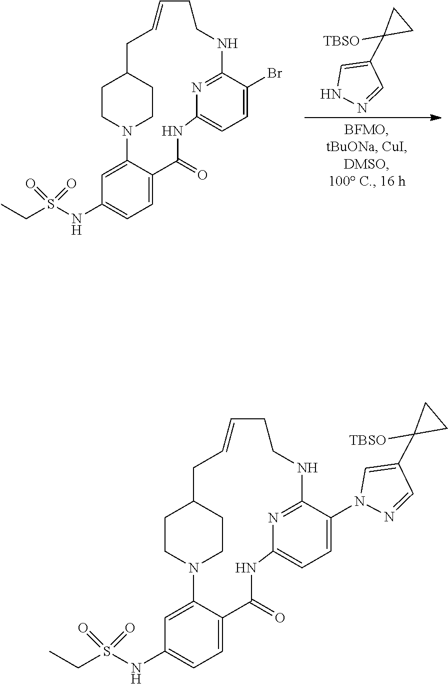 [SMILES: CCS(=O)(=O)Nc1ccc2c(c1)N1CCC(C/C=C/CCNc3nc(ccc3Br)NC2=O)CC1 ; CC(C)(C)[Si](C)(C)OC1(c2cn[nH]c2)CC1 ; CCS(=O)(=O)Nc1ccc2c(c1)N1CCC(C/C=C/CCNc3nc(ccc3-n3cc(C4(O[Si](C)(C)C(C)(C)C)CC4)cn3)NC2=O)CC1]

t-BuONa (26 mg, 0.27 mmol) was added to the mixture of (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (50 mg, 0.091 mmol, Compound 304), 4-(1-((tert-butyldimethylsilyl)oxy)cyclopropyl)-1H-pyrazole (65 mg, 0.27 mmol), CuI (2 mg, 0.01 mmol) and N¹,N²-bis(furan-2-ylmethyl)oxalamide (3 mg, 0.01 mmol) in DMSO (5 mL) under nitrogen atmosphere. The mixture was stirred at 100° C. overnight. After cooling to room temperature, the reaction mixture was diluted with H₂O (50 mL) and extracted with EA (3×50 mL). The combined organic layers were washed with brine (200 mL), dried over anhydrous Na₂SO₄, filtered and concentrated under vacuum to afford the crude product. The crude product purified by reversed-phase flash chromatography, with the following conditions: Column, C18 silica gel; mobile phase, aq. 0.01% FA in ACN, 10% to 100% gradient in 10 min; detector, UV 254 nm to afford (E)-N-(5⁵-(4-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (25 mg, 39% yield). LCMS (ESI-MS) m/z=706.3 [M+H]⁺.

### Step 2: (E)-N-(5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl) ethanesulfonamide

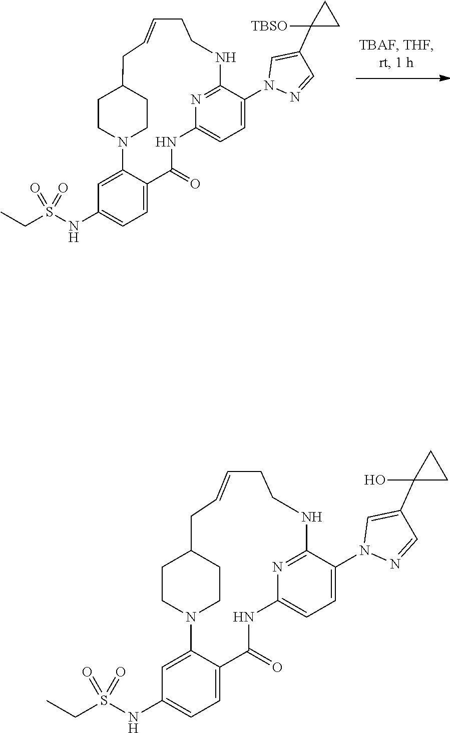 [SMILES: CCS(=O)(=O)Nc1ccc2c(c1)N1CCC(C/C=C/CCNc3nc(ccc3-n3cc(C4(O[Si](C)(C)C(C)(C)C)CC4)cn3)NC2=O)CC1 ; CCS(=O)(=O)Nc1ccc2c(c1)N1CCC(C/C=C/CCNc3nc(ccc3-n3cc(C4(O)CC4)cn3)NC2=O)CC1]

1M TBAF in THF (0.07 mL, 0.07 mmol) was added to (E)-N-(5⁵-(4-(1-((tert-butyldimethylsilyl) oxy) cyclopropyl)-1H-pyrazol-1-yl)-3-oxo-4,6-diaza-5(2,6)-pyridina-1(1,4)-piperidina-2(1,2)-benzenac-ycloundecaphan-9-en-2⁵-yl) ethanesulfonamide (20 mg, 0.03 mmol) in THF (3 mL). The mixture was stirred at room temperature for 1 hour. The reaction was poured into aq. NH₄Cl (10 mL) at room temperature. The resulting mixture was extracted with EA (10 mL). The combined organic layers were washed with aq. NH₄Cl (2×10 mL), dried over anhydrous Na₂SO₄. After filtration, the filtrate was concentrated under reduced pressure. The crude product purified by silica gel column chromatography, eluted with DCM:MeOH (20:1) to afford (E)-N-(5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl) ethanesulfonamide (9.8 mg, 53% yield).

Compound 283 was prepared according to General Procedure 2 using Compound 304 and 1-methyl-4-(1H-pyrazol-4-yl)piperidine to afford (E)-N-(5⁵-(4-(1-methylpiperidin-4-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (7.4 mg, 32% yield).

Compound 284 was prepared according to General Procedure 2 using Compound 304 and [1-(1H-pyrazol-3-yl)cyclopentyl]methanol to afford (E)-N-(55-(3-(1-(hydroxymethyl)cyclopentyl)-1H-pyrazol-1-yl)-3-oxo-4,6-diaza-5(2,6)-pyridina-1(1,4)-piperidina-2(1,2)-benzenacycloundecaphan-9-en-25-yl)ethanesulfonamide (10.3 mg, 37.2% yield).

Compound 285 was prepared according to General Procedure 2 using Compound 304 and [1-(1H-pyrazol-3-yl)cyclobutyl]methanol to afford (E)-N-(55-(3-(1-(hydroxymethyl)cyclobutyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.5 mg, 20.4% yield).

Compound 286 was prepared according to General Procedure 2 using Compound 311 and 1-(1H-pyrazol-3-yl)pyrrolidin-2-one to afford (E)-N-(1⁴-methyl-3-oxo-5⁵-(3-(2-oxopyrrolidin-1-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (0.5 mg, 1.8% yield).

Compound 287 was prepared according to General Procedure 2 using Compound 304 and 1-methyl-4-(1H-pyrazol-4-yl)piperidine to afford (E)-N-(5⁵-(4-(morpholine-4-carbonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (7.4 mg, 31.7% yield).

Compound 288 was prepared according to General Procedure 2 using Compound 310 and 4-(oxolan-3-yl)-1H-pyrazole to afford (Z)—N-(1⁴-methyl-3-oxo-5⁵-(4-(tetrahydrofuran-3-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (6 mg, 22.7% yield).

Compound 289 was prepared according to General Procedure 2 using Compound 310 and 4-(oxolan-3-yl)-1H-pyrazole to afford (Z)—N-(1⁴-methyl-3-oxo-5⁵-(4-(tetrahydrofuran-3-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (10.9 mg, 47.6% yield).

Compound 290 was prepared according to General Procedure 2 using Compound 310 phenyl(1H-pyrazol-4-yl)methanol to afford (Z)—N-(5⁵-(4-(hydroxy(phenyl)methyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-25-yl)ethanesulfonamide (11.7 mg, 48.1% yield).

Compound 291 was prepared according to General Procedure 2 using Compound 311 phenyl(1H-pyrazol-4-yl)methanol to afford (E)-N-(5⁵-(4-(hydroxy(phenyl)methyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (8.8 mg, 36.2% yield).

Compound 292 was prepared according to General Procedure 2 using Compound 304 4-cyclopropylmethoxy-1H-pyrazole to afford (E)-N-(5⁵-(4-(cyclopropylmethoxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (10.2 mg, 38.4% yield).

Compound 293 was prepared according to General Procedure 2 using Compound 310 and 4-cyclopropylmethoxy-1H-pyrazole to afford (Z)—N-(5⁵-(4-(cyclopropylmethoxy)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-25-yl)ethanesulfonamide (10.6 mg, 46.3% yield).

Compound 294 was prepared according to General Procedure 2 using Compound 300 and 4-(cyclopropanesulfonyl)-1H-pyrazole to afford (E)-5⁵-(4-(cyclopropylsulfonyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (1.7 mg, 7.5% yield).

Compound 295 was prepared according to General Procedure 2 using Compound 311 1-(1H-pyrazol-4-yl)pyrrolidin-2-one to afford (E)-N-(1⁴-methyl-3-oxo-5⁵-(4-(2-oxopyrrolidin-1-yl)-1H-pyrazol-1-yl)-11-oxa-4,6-diaza-5(2,6)-pyridina-1(1,4)-piperidina-2(1,2)-benzenacycloundecaphan-8-en-25-yl)ethanesulfonamide (10.4 mg, 37.6% yield).

Compound 296 was prepared according to General Procedure 2 using Compound 311 4-(cyclopentyloxy)-1H-pyrazole to afford (E)-N-(5⁵-(4-(cyclopentyloxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (8.8 mg, 37.5% yield).

Compound 297 was prepared according to General Procedure 2 using Compound 311 and [1-(1H-pyrazol-3-yl)cyclopentyl]methanol to afford (E)-N-(55-(3-(1-(hydroxymethyl)cyclopentyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (10.1 mg, 41.8% yield).

Compound 298 was prepared according to General Procedure 2 using Compound 310 4-cyclobutoxy-1H-pyrazole to afford (Z)—N-(5⁵-(4-cyclobutoxy-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (11.5 mg, 42.2% yield).

### Compound 299.

### Step 1A: 4-(1-trityl-1H-pyrazol-4-yl)morpholine

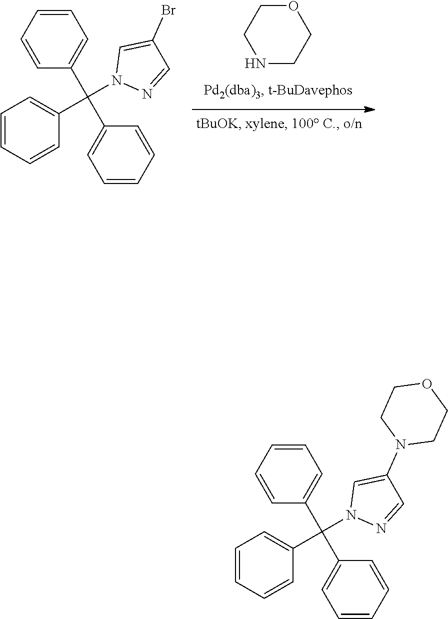 [SMILES: Brc1cnn(C(c2ccccc2)(c2ccccc2)c2ccccc2)c1 ; C1COCCN1 ; c1ccc(C(c2ccccc2)(c2ccccc2)n2cc(N3CCOCC3)cn2)cc1]

Pd₂(dba)₃ (23.5 g, 25.7 mmol) and t-BuDavephos (17.5 g, 51.4 mmol) were added to the mixture of 4-bromo-1-trityl-1H-pyrazole (100 g, 257 mmol), morpholine (44.8 g, 514 mmol) and tBuOK (57.6 g, 514 mmol) in xylene (500 mL) at room temperature under nitrogen atmosphere. The resulting mixture was stirred for overnight at 100° C. under nitrogen atmosphere. The reaction mixture was diluted with water (1 L) and extracted with EA (3×1 L). The combined organic layers were washed with brine (3 L), dried over anhydrous Na₂SO₄, filtered and concentrated under vacuum to afford the crude product. The crude product was dissolved in DCM. Then the solution was uploaded to Flash Column Silica-CS (330 g) and eluting with EA in PE from 0% to 40% with 100 mL/min flow rate. Fractions with desired mass signal were combined and concentrated under vacuum to afford the desired product 4-(1-trityl-1H-pyrazol-4-yl)morpholine (65 g, 45% yield). LCMS (ESI-MS) m/z=396.1 [M+H]⁺.

### Step 2A: 4-(1H-pyrazol-4-yl)morpholine

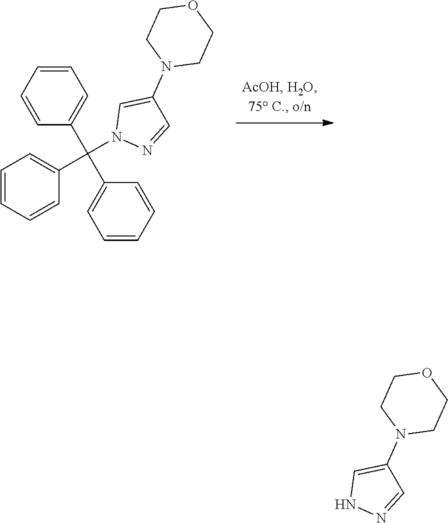 [SMILES: c1ccc(C(c2ccccc2)(c2ccccc2)n2cc(N3CCOCC3)cn2)cc1 ; c1n[nH]cc1N1CCOCC1]

AcOH (1200 mL) was added to the mixture of 4-(1-trityl-1H-pyrazol-4-yl)morpholine (64 g, 0.16 mol) in H₂O (600 mL) at 25° C. Then the resulting mixture was stirred at 75° C. for overnight. The mixture was basified to Ph=8 with aq. NaOH. The aqueous layer was extracted with EA (3×1 L) and dried over anhydrous Na₂SO₄, filtered and concentrated under vacuum to afford the crude product. The crude product was dissolved in DCM. Then the solution was uploaded to Flash Column Silica-CS (330 g) and eluting with MeOH in DCM from 0% to 20% with 100 mL/min flow rate. Fractions with desired mass signal were combined and concentrated under vacuum to afford the desired product 4-(1H-pyrazol-4-yl)morpholine (9.5 g, 34% yield). LCMS (ESI-MS) m/z=154.0 [M+H]⁺.

### Step 3A: N²-(but-3-en-1-yl)-3-(4-morpholino-1H-pyrazol-1-yl)pyridine-2,6-diamine

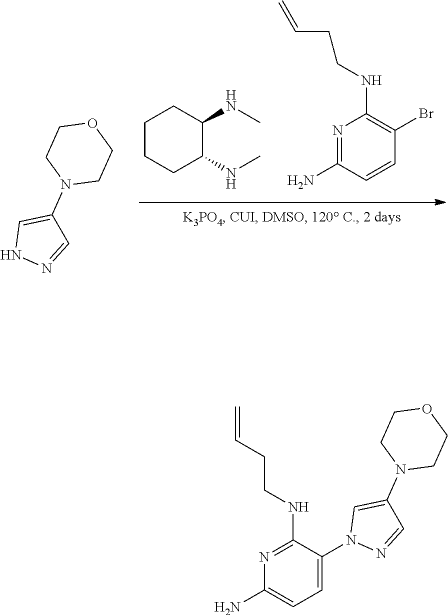

CuI (1.0 g, 5.4 mmol) and (1R,2R)—N¹,N²-dimethylcyclohexane-1,2-diamine (0.76 g, 5.4 mmol) were added to the mixture of 3-bromo-N²-(but-3-en-1-yl)pyridine-2,6-diamine (13 g, 54 mmol), 4-(1H-pyrazol-4-yl)morpholine (8.2 g, 54 mmol) and K₃PO₄(34 g, 160 mmol) in DMSO (100 mL) under nitrogen atmosphere at room temperature. Then the resulting mixture was stirred at 120° C. for 2 days. After cooling to room temperature, the reaction mixture was diluted with water (1 L) and extracted with EA (3×1 L). The combined organic layers were washed with brine (3 L), dried over anhydrous Na₂SO₄, filtered, and concentrated under vacuum to afford the crude product. The crude product was dissolved in DCM and was purified via flash column chromatography (Column Silica-CS (330 g) and eluting with EA in PE from 0% to 40% with 100 mL/min flow rate). Fractions with desired mass signal were combined and concentrated under vacuum to afford N²-(but-3-en-1-yl)-3-(4-morpholino-1H-pyrazol-1-yl)pyridine-2,6-diamine (8 g, 40% yield). LCMS (ESI-MS) m/z=315.0 [M+H]⁺.

### Step 1B: tert-butyl 4-allylpiperidine-1-carboxylate

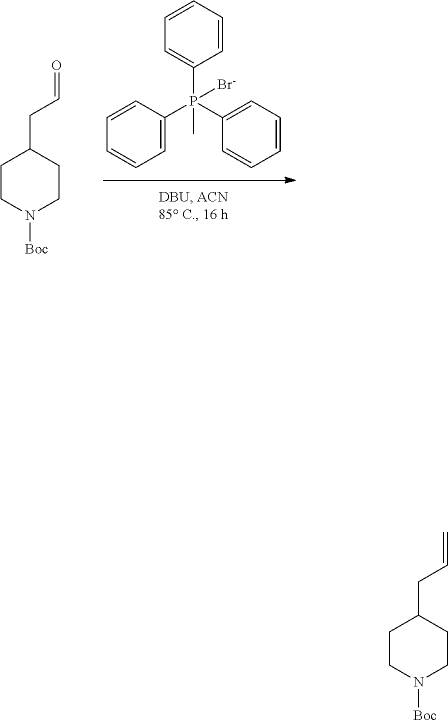

Into a solution of tert-butyl 4-(2-oxoethyl)piperidine-1-carboxylate (20 g, 8 mmol) in ACN (200 mL) was added methyltriphenylphosphonium bromide (62.8 g, 176 mmol) and DBU (26.8 g, 176 mmol). The resulting solution was stirred at 85° C. for 16 hours. The solution was concentrated under vacuum to afford the crude product. The reaction was quenched with water (100 mL). The aqueous layer was extracted with EA (3×100 mL), and wash the organic phase with saturated saline solution (300 mL). The resulting mixture was dried with Na₂SO₄. The resulting mixture was concentrated under reduced pressure. The residue was purified by silica gel column chromatography, eluted with EA/PE (1:5) to afford tert-butyl 4-allylpiperidine-1-carboxylate (12 g, 48% yield). LCMS (ESI-MS) m/z=226.8 [M+H]⁺.

### Step 1B: 4-allylpiperidine

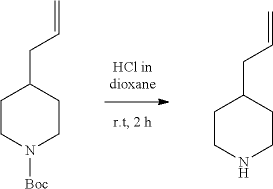

Into a 250 mL round-bottom flask were added tert-butyl 4-allylpiperidine-1-carboxylate (12 g, 53 mmol) and HCl in dioxane (60 mL) at room temperature. The solution was stirred for 2 hours at room temperature. The solution was concentrated under reduced pressure. The crude product 4-allylpiperidine (9 g, 90% yield) was used in the next step directly without further purification. LCMS (ESI-MS) m/z=126.1 [M+H]⁺.

### Step 2B: methyl 2-(4-allylpiperidin-1-yl)-4-bromobenzoate

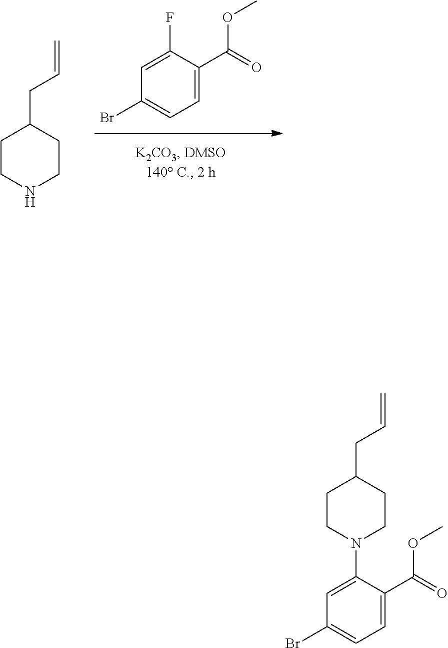

Into a 500 mL round-bottom flask were added 4-allylpiperidine (9 g, 70 mmol) in DMSO (90 mL), then added K₂CO₃ (30.4 g, 220 mmol) and methyl 4-bromo-2-fluorobenzoate (17 g, 3 mmol) at room temperature. The resulting mixture was stirred for 2 hours at 140° C. The resulting mixture was extracted with EA (3×10 mL). The combined organic layers were washed with water (3×10 mL), dried over anhydrous Na₂SO₄. After filtration, the filtrate was concentrated under reduced pressure. The residue was purified by silica gel column chromatography, eluted with EA/PE (1:1) to afford methyl 2-(4-allylpiperidin-1-yl)-4-bromobenzoate (12 g, 39% yield). LCMS (ESI-MS) m/z=338.0 [M+H]⁺.

### Step 3B: 2-(4-allylpiperidin-1-yl)-4-bromobenzoic acid

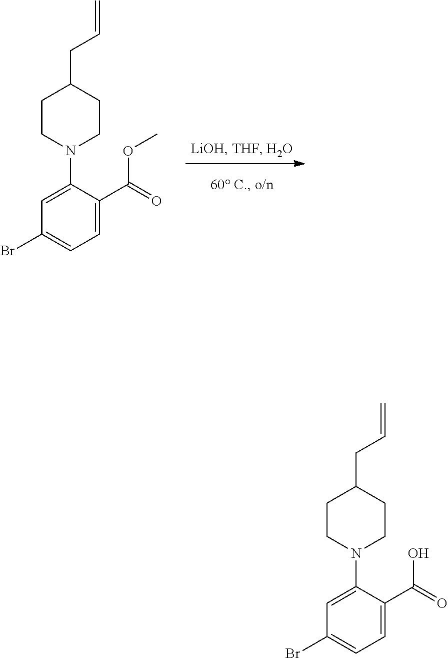 [SMILES: C=CCC1CCN(c2cc(Br)ccc2C(=O)OC)CC1 ; C=CCC1CCN(c2cc(Br)ccc2C(=O)O)CC1]

Into a 500 mL round-bottom flask were added methyl 2-(4-allylpiperidin-1-yl)-4-bromobenzoate (12 g, 35 mmol) in THF (120 mL) and H₂O (40 mL), then added LiOH (6.9 g, 290 mmol) at 60° C. for overnight. The mixture was acidified to pH 3 with 2M HCl (aq.). The combined organic layers were concentrated under reduced pressure. The residue was purified by silica gel column chromatography, eluted with PE/EA (1:1) to afford 2-(4-allylpiperidin-1-yl)-4-bromobenzoic acid (10 g, 70% yield). LCMS (ESI-MS) m/z=324.0 [M+H]⁺.

### Step 4. 2-(4-allylpiperidin-1-yl)-4-bromo-N-(6-(but-3-en-1-ylamino)-5-(4-morpholino-1H-pyrazol-1-yl)pyridin-2-yl)benzamide

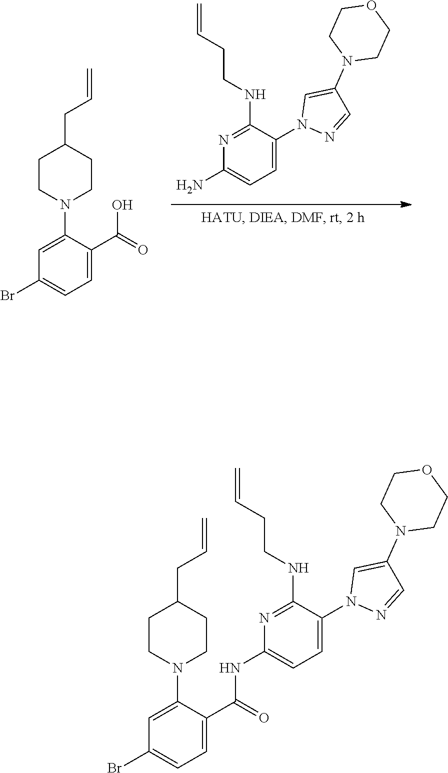 [SMILES: C=CCC1CCN(c2cc(Br)ccc2C(=O)O)CC1 ; C=CCCNc1nc(N)ccc1-n1cc(N2CCOCC2)cn1 ; C=CCCNc1nc(NC(=O)c2ccc(Br)cc2N2CCC(CC=C)CC2)ccc1-n1cc(N2CCOCC2)cn1]

To a stirred solution of 2-(4-allylpiperidin-1-yl)-4-bromobenzoic acid (7 g, 20.0 mmol) in DMF (70 mL) was added N²-(but-3-en-1-yl)-3-(4-morpholino-1H-pyrazol-1-yl)pyridine-2,6-diamine (8 g, 30 mmol), DIEA (8 g, 60 mmol) and HATU (12.4 g, 32.6 mmol) at room temperature. The reaction mixture was stirred at room temperature for a period of 2 hours. The reaction mixture was diluted with water (500 mL) and extracted with EA (3×500 mL). The combined organic layers were washed with brine (1500 mL), dried over anhydrous Na₂SO₄, filtered and concentrated under vacuum to afford the crude product. The crude product which was further purified by column chromatography using 10% to 90% EA in PE gradient to afford desired compound 2-(4-allylpiperidin-1-yl)-4-bromo-N-(6-(but-3-en-1-ylamino)-5-(4-morpholino-1H-pyrazol-1-yl)pyridin-2-yl)benzamide (12 g, 90% yield). LCMS (ESI-MS) m/z=622.25 [M+H]⁺.

### Step 5: Compound 299

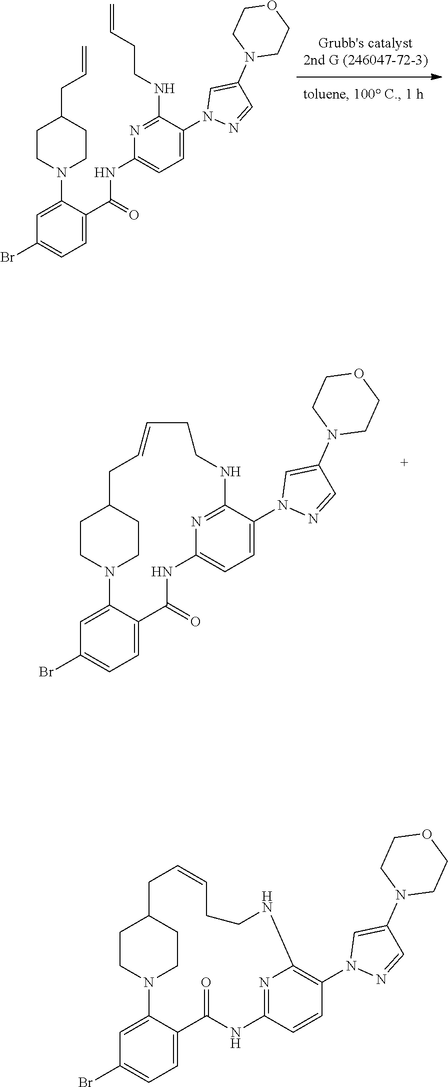 [SMILES: C=CCCNc1nc(NC(=O)c2ccc(Br)cc2N2CCC(CC=C)CC2)ccc1-n1cc(N2CCOCC2)cn1 ; O=C1Nc2ccc(-n3cc(N4CCOCC4)cn3)c(n2)NCC/C=C/CC2CCN(CC2)c2cc(Br)ccc21 ; O=C1Nc2ccc(-n3cc(N4CCOCC4)cn3)c(n2)NCC/C=C\CC2CCN(CC2)c2cc(Br)ccc21]

A mixture of 2-(4-allylpiperidin-1-yl)-4-bromo-N-(6-(but-3-en-1-ylamino)-5-(4-morpholino-1H-pyrazol-1-yl)pyridin-2-yl)benzamide (12 g, 19 mmol) and benzylidene(dichloro)(1,3-dimesityl-2-imidazolidinylidene)ruthenium-tricyclohexylphosphine (1:1) (1.6 g, 1.9 mmol) in toluene (120 mL) was stirred at 100° C. for 1 hour under nitrogen atmosphere. The resulting mixture was concentrated under vacuum and the resulting residue was purified by silica gel column chromatography, eluted with PE/EA (5:1) to afford the crude product (6 g, purity 85%). This material was further purified by preparative SFC with the conditions (Column: (R, R)-Whelk-O 1 4.6*50 mm, 3 sm; Mobile Phase B: DCM:MeOH=1:1 (1% 2M NH₃-MeOH); Flow rate: 4 mL/min; Gradient (B %): isocratic % B) to afford (E)-2⁵-bromo-5⁵-(4-morpholino-1H-pyrazol-1-yl)-4,6-diaza-5(2,6)-pyridina-1(1,4)-piperidina-2(1,2)-benzenacycloundecaphan-9-en-3-one (2.608 g, 3.39 mmol, 18% yield) and (Z)-2⁵-bromo-5⁵-(4-morpholino-1H-pyrazol-1-yl)-4,6-diaza-5(2,6)-pyridina-1(1,4)-piperidina-2(1,2)-benzenacycloundecaphan-9-en-3-one (0.6574 g, 1.08 mmol, 5.6% yield).

### Compound 300.

### Step 1: 4-allylpiperidine hydrochloride

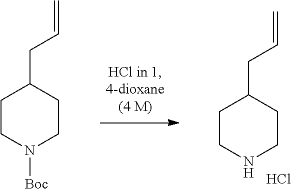

A reactor was charged with tert-butyl 4-allylpiperidine-1-carboxylate (150 g, 667 mmol, 1.0 eq.) and 1,4-dioxane (1.5 L, 10V). The, HCl in 1,4-dioxane (500 mL, 2.0 mol, 3.0 eq., 4.0M) was added in the reaction at 5-10° C. After stirring overnight at 20-25° C., the solution was concentrated under reduced pressure and the solved was exchanged for MTBE (2×5 V with drying). Additional MTBE (10 V) was added, and the mixture was stirred for 30 min. The resulting slurry was filtered and collected to afford 4-allylpiperidine hydrochloride (93 g, 87% yield).

### Step 2: methyl 2-(4-allylpiperidin-1-yl)-4-iodobenzoate

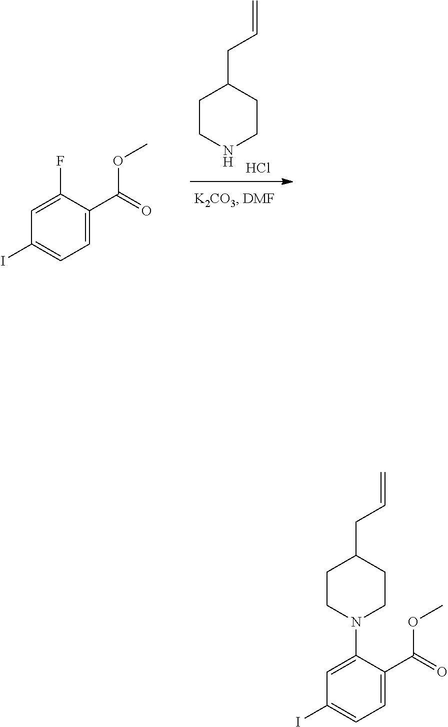

A flask was charged with methyl 2-fluoro-4-iodobenzoate (22 g, 78.6 mmol, 1 equiv), 4-allylpiperidine hydrochloride (12.6 g, 78.6 mmol, 1 equiv), K₂CO₃ (32.5 g, 236 mmol, 3 equiv) and DMF (220 mL). After stirring overnight at 80° C., the reaction mixture was cooled to room temperature, was filtered through celite, and was quenched with saturated NH₄Cl aqueous solution (10 V). The mixture was extracted with EA (3×10 V), the combined organic layers were washed with saturated NaCl aqueous solution (2×10 V), and the organic was then dried over Na₂SO₄. The solution was concentrated under reduced pressure to afford methyl 2-(4-allylpiperidin-1-yl)-4-iodobenzoate (24.1 g, 79.6% yield). LCMS: (ES, m/z): 386[M+H]+

### Step 3: methyl 4-(ethylsulfonyl)-2-fluorobenzoate

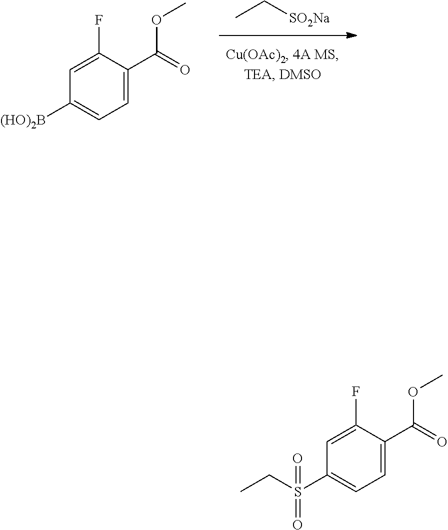

(3-fluoro-4-(methoxycarbonyl)phenyl)boronic acid (100 g, 505 mmol, 1 equiv), sodium ethanesulfinate (87.8 g, 758 mmol, 1.5 equiv), Cu(OAc)₂ (101 g, 556 mmol, 1.1 equiv), TEA (102 g, 1.01 mol, 2 equiv), Molecular sieves (200 g, 200% w/w) and DMSO (1000 mL) were added into a flask at 25 ±5° C. The mixture was stirred overnight at 30° C. The reaction was filtered, quenched by the addition of H₂O (10 V) at 25±5° C., extracted with EA (3×10 V). The combined organic layers were washed with saturated NaCl aqueous solution (2×10 V), and the combined organic layers were then dried over Na₂SO₄. The solution was concentrated under reduced pressure and the resulting residue was purified by column chromatography on silica gel to afford methyl 4-(ethylsulfonyl)-2-fluorobenzoate (78 g, 62% yield). LCMS: (ES, m/z): 264[M+NH₃]+

### Step 4: methyl 2-(4-allylpiperidin-1-yl)-4-(ethylsulfonyl)benzoate

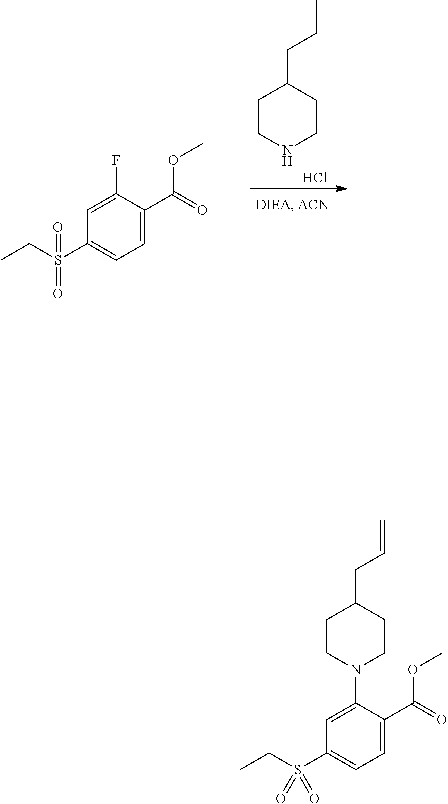

Methyl 4-(ethylsulfonyl)-2-fluorobenzoate (50 g, 203 mmol, 1 equiv), 4-allylpiperidine hydrochloride (32.5 g, 203 mmol, 1 equiv), DIEA (78.5 g, 610 mmol, 3 equiv) and ACN (500 mL) were added into a flask at 25±5° C. The mixture was stirred overnight at 70° C. The mixture was concentrated in vacuum to 2-3V, was added to H₂O (10 V) at 25±5° C., and the resulting solution was extracted with EA (3×10 V). And the organic was dried over Na₂SO₄. The solution was concentrated under reduced pressure to afford methyl 2-(4-allylpiperidin-1-yl)-4-(ethylsulfonyl)benzoate (70 g, 95% yield). LCMS: (ES, m/z): 352[M+H]+

### Step 5: 2-(4-allylpiperidin-1-yl)-4-(ethylsulfonyl)benzoic acid

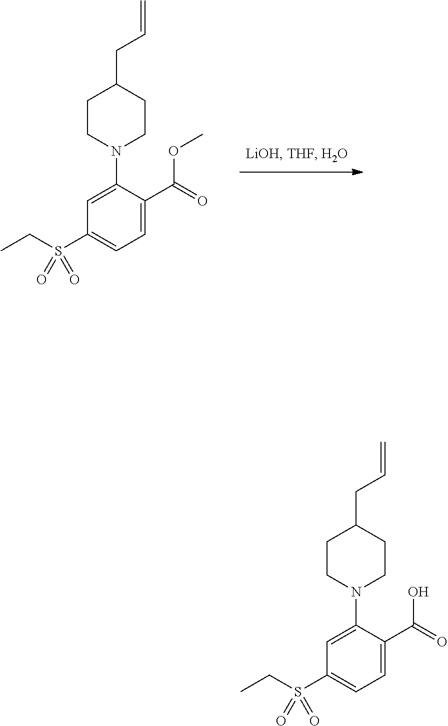 [SMILES: C=CCC1CCN(c2cc(S(=O)(=O)CC)ccc2C(=O)OC)CC1 ; C=CCC1CCN(c2cc(S(=O)(=O)CC)ccc2C(=O)O)CC1]

Methyl 2-(4-allylpiperidin-1-yl)-4-(ethylsulfonyl)benzoate (70 g, 199 mmol, 1 equiv), LiOH (15.3 g, 598 mmol, 3 equiv), THF (470 mL) and H₂O (230 mL) were added into a flask at 25±5° C. The mixture was stirred overnight at 60° C. After cooling to room temperature, HCl (3M, 213 mL, 3 equiv) was added at 25±5° C. The mixture was quenched with a saturated NH₄Cl solution (10 V), was extracted with EA (3×10 V), and the organic phase was then dried over Na₂SO₄, filtered, and concentrated under reduced pressure to afford 2-(4-allylpiperidin-1-yl)-4-(ethylsulfonyl)benzoic acid (60 g, 89% yield). LCMS: (ES, m/z): 338[M+H]+

### Step 6: N-[5-bromo-6-(but-3-en-1-ylamino)pyridin-2-yl]-4-(ethanesulfonyl)-2-[4-(prop-2-en-1-yl)piperidin-1-yl]benzamide

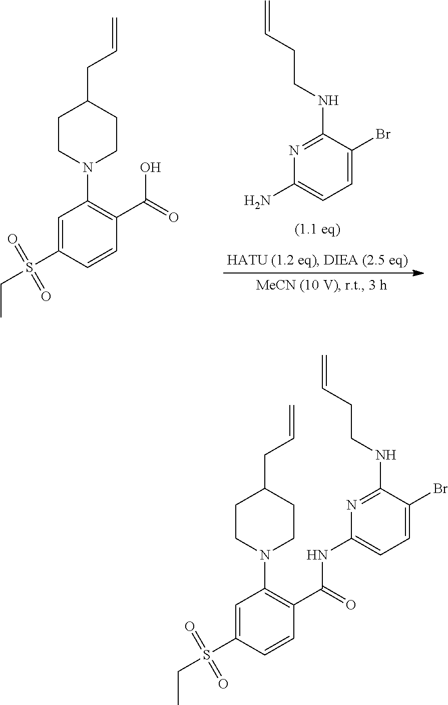 [SMILES: C=CCC1CCN(c2cc(S(=O)(=O)CC)ccc2C(=O)O)CC1 ; C=CCCNc1nc(N)ccc1Br ; C=CCCNc1nc(NC(=O)c2ccc(S(=O)(=O)CC)cc2N2CCC(CC=C)CC2)ccc1Br]

To a solution of 4-(ethanesulfonyl)-2-[4-(prop-2-en-1-yl)piperidin-1-yl]benzoic acid (10 g, 29.6 mmol, 1.0 equiv) in MeCN (100 mL) was added 3-bromo-N²-(but-3-en-1-yl)pyridine-2,6-diamine (7.89 g, 32.6 mmol, 1.1 equiv), HATU (13.5 g, 35.5 mmol, 1.2 equiv) and DIEA (9.58 g, 74.1 mmol, 2.5 equiv) at 25° C. The reaction was stirred at 25° C. for 3 h. The desired product could be detected by LCMS. The reaction mixture was concentrated under vacuum. The residue was purified by silica gel column chromatography (eluted with PE/EA (5:1)) to afford N-[5-bromo-6-(but-3-en-1-ylamino)pyridin-2-yl]-4-(ethanesulfonyl)-2-[4-(prop-2-en-1-yl)piperidin-1-yl]benzamide (15.8 g, 95% yield). LCMS: (ES, m z): [M+1]⁺: 563

### Step 7: 5⁵-bromo-2⁵-(ethylsulfonyl)-4,6-diaza-5(2,6)-pyridina-1(1,4)-piperidina-2(1,2)-benzenacycloundecaphan-9-en-3-one

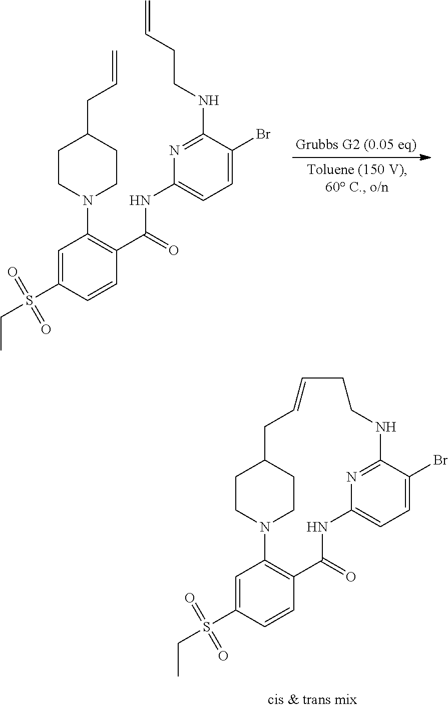 [SMILES: C=CCCNc1nc(NC(=O)c2ccc(S(=O)(=O)CC)cc2N2CCC(CC=C)CC2)ccc1Br ; CCS(=O)(=O)c1ccc2c(c1)N1CCC(C/C=C/CCNc3nc(ccc3Br)NC2=O)CC1]

A solution of N-[5-bromo-6-(but-3-en-1-ylamino)pyridin-2-yl]-4-(ethanesulfonyl)-2-[4-(prop-2-en-1-yl)piperidin-1-yl]benzamide (15.8 g, 28.1 mmol, 1.0 equiv) in toluene (2370 mL) was treated with Grubbs G2 (1.19 g, 1.40 mmol, 0.05 equiv) at room temperature under a nitrogen atmosphere. The reaction was stirred at 60° C. for 16 h. The mixture was allowed to cool down to room temperature and imidazole (2.30 g, 33.7 mmol, 1.2 equiv) was added at room temperature to quench the Grubbs catalyst. The resulting mixture was stirred at room temperature for additional 3 h. The resulting mixture was concentrated under reduced pressure and the resulting residue was purified by silica gel column chromatography (eluted with PE/EA (2:1)) to afford a cis- and trans- mixture of (E) and (Z)-5⁵-bromo-2⁵-(ethylsulfonyl)-4,6-diaza-5(2,6)-pyridina-1(1,4)-piperidina-2(1,2)-benzenacycloundecaphan-9-en-3-one (10.3 g, 68.6% yield, 1:5 cis:trans ratio).

### Step 8: (E)-5⁵-bromo-2⁵-(ethylsulfonyl)-4,6-diaza-5(2,6)-pyridina-1(1,4)-piperidina-2(1,2)-benzenacycloundecaphan-9-en-3-one (Compound 300)

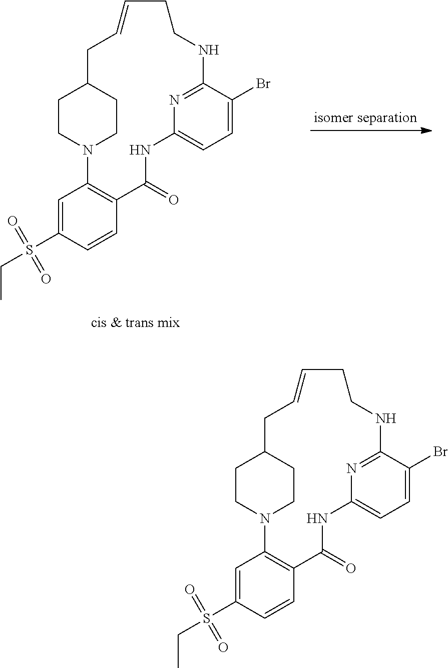 [SMILES: CCS(=O)(=O)c1ccc2c(c1)N1CCC(C/C=C/CCNc3nc(ccc3Br)NC2=O)CC1 ; CCS(=O)(=O)c1ccc2c(c1)N1CCC(C/C=C/CCNc3nc(ccc3Br)NC2=O)CC1]

Cis- and trans- mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (10.3 g, 19.3 mmol, 1.0 equiv) was purified by preparative achiral-SFC with the following conditions: Column: GreenSep Naphthyl 3*25 cm, 5 μm; Mobile Phase A: CO₂, Mobile Phase B: IPA (0.1% 7M NH₃-MeOH); Flow rate: 100 mL/min; Gradient (B %): isocratic 50% B; Back Pressure(bar): 100; Wave Length: 220 nm; RT1 (min): 4.2; RT2 (min): 4.6; Total Elution Time (min): 6; Sample Solvent: MEOH; Injection Volume: 0.5 mL. The resulting solution was concentrated under vacuum. The resulting mixture was dried by lyophilization with acetonitrile and water overnight to afford (E)-5⁵-bromo-2⁵-(ethylsulfonyl)-4,6-diaza-5(2,6)-pyridina-1(1,4)-piperidina-2(1,2)-benzenacycloundecaphan-9-en-3-one (5.4358 g, 52.8% yield)

### Compounds 301 and 302.

### Step 1: tert-butyl 4-(allyloxy)-4-methylpiperidine-1-carboxylate

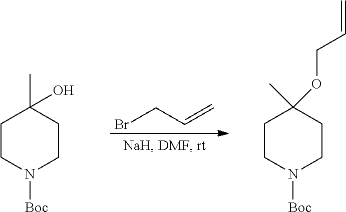

A reactor was charged with NaH (152 g, 6.32 mol, 2 equiv) under a nitrogen atmosphere. The reactor was cooled to 0±5° C. and DMF (5 V) was added. A solution of tert-butyl 4-hydroxy-4-methylpiperidine-1-carboxylate (680 g, 3.16 mol, 1 equiv) in DMV (2 V) was added dropwise at 0±5° C. The mixture was stirred for 0.5 h at this temperature, then allyl bromide (1.91 kg, 15.8 mol, 5 equiv) was slowly added dropwise at 0±5° C. The mixture was stirred for 3 h at 25±5° C. The reaction was cooled to 0° C., quenched with saturated NH₄Cl aqueous solution (5 V), and extracted with MTBE (3×10 V). The combined organic layers were washed with saturated NaCl aqueous solution (2×10 V), and the organic layer was then dried over Na₂SO₄. The solution was concentrated under reduced pressure and purified by column chromatography on silica gel to afford tert-butyl 4-(allyloxy)-4-methylpiperidine-1-carboxylate (686 g, 85% yield).

### Step 2: 4-methyl-4-(prop-2-en-1-yloxy) piperidine hydrochloride

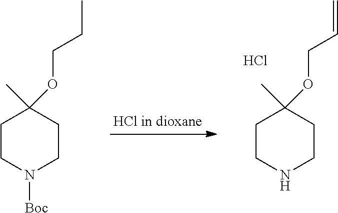 [SMILES: CCCOC1(C)CCN(C(=O)OC(C)(C)C)CC1 ; Cl ; C=CCOC1(C)CCNCC1]

A cooled reactor was sequentially charged with tert-butyl 4-methyl-4-(prop-2-en-1-yloxy)piperidine-1-carboxylate (580 g, 2.27 mol, 1 equiv) and HCl in dioxane (1.70 L, 48.3 mmol, 3 equiv, dropwise, 0 5° C.). The solution was allowed to warm to room temperature and the mixture was stirred for 3 h. After this time, the mixture concentrated under reduced pressure, MTBE (5 V) was added, and the slurry was stirred overnight at room temperature. The mixture was filtered to obtain 4-methyl-4-(prop-2-en-1-yloxy) piperidine hydrochloride (395 g, 91% yield).

### Step 3: methyl 2-(4-(allyloxy)-4-methylpiperidin-1-yl)-4-iodobenzoate

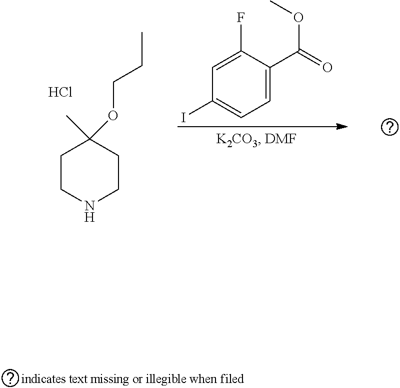

4-methyl-4-(prop-2-en-1-yloxy) piperidine hydrochloride (20 g, 104 mmol, 1 equiv), methyl 2-fluoro-4-iodobenzoate (29.2 g, 104 mmol, 1 equiv), K₂CO₃ (43.3 g, 313 mmol, 3 equiv) and DMF (200 mL) were added into a flask at 25±5° C. The mixture was stirred overnight at 60° C. The reaction was quenched by the addition of H₂O at 25±5° C. The resulting mixture was extracted with MTBE (10 V×3). The reaction was quenched by the addition of H₂O (10 V) at 25±5° C. The resulting mixture was extracted with MTBE (10 V×3). The combined organic layers were washed with saturated NaCl aqueous solution (10 V×2), dried over anhydrous Na₂SO₄. After filtration, the filtrate was concentrated under reduced pressure, and the crude material was purified by column chromatography on silica gel to afford methyl 2-(4-(allyloxy)-4-methylpiperidin-1-yl)-4-iodobenzoate (30 g, 70% yield).

### Step 4: methyl 2-(4-(allyloxy)-4-methylpiperidin-1-yl)-4-(ethylsulfonyl)benzoate

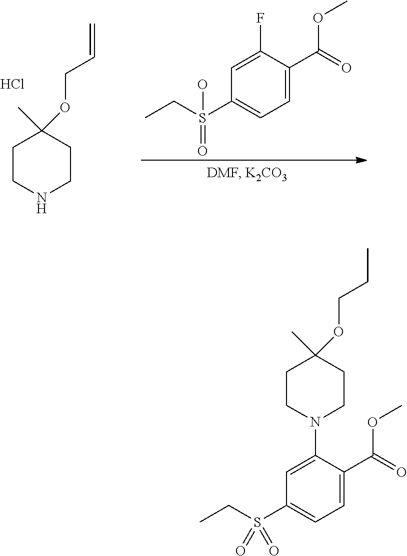

Methyl 4-(ethanesulfonyl)-2-fluorobenzoate (60 g, 244 mmol, 1 equiv). 4-methyl-4-(prop-2-en-1-yloxy) piperidine hydrochloride (51.4 g, 268 mmol, 1.1 equiv), K₂CO₃ (101 g, 731 mmol, 3 equiv) and DMF (600 mL) were added into a flask at 25±5° C. The mixture was stirred overnight at 60° C. After cooling to room temperature, the reaction was quenched by the addition of H₂O (10 V) at 25±5° C. The resulting mixture was extracted with MTBE (10 V×3). The combined organic layers were washed with saturated NaCl aqueous solution (10 V×2), dried over anhydrous Na₂SO₄. After filtration, the filtrate was concentrated under reduced pressure. The crude product was used in the next step directly without further purification.

### Step 5: 4-(ethanesulfonyl)-2-[4-methyl-4-(prop-2-en-1-yloxy)piperidin-1-yl]benzoic acid

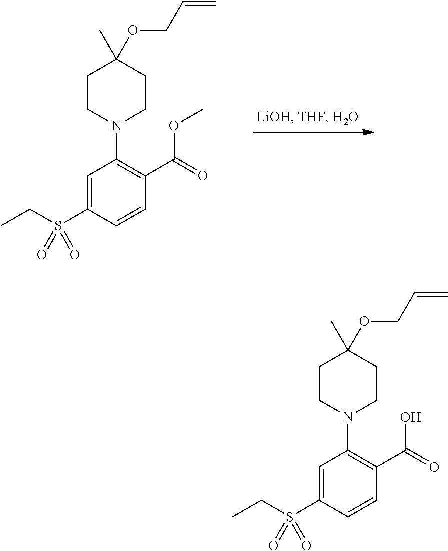 [SMILES: C=CCOC1(C)CCN(c2cc(S(=O)(=O)CC)ccc2C(=O)OC)CC1 ; C=CCOC1(C)CCN(c2cc(S(=O)(=O)CC)ccc2C(=O)O)CC1]

Methyl 4-(ethanesulfonyl)-2-[4-methyl-4-(prop-2-en-1-yloxy)piperidin-1-yl]benzoate (90 g, 236 mmol, 1 equiv), LiOH·H₂O (49.5 g, 1.18 mol, 5 equiv), THF (600 mL) and H₂O (300 mL) were added into a flask at 25±5° C. The mixture was stirred overnight at 60° C. The reaction was quenched by the addition of H₂O (15 V) at 25±5° C. The aqueous layer was extracted with MTBE (10 V×3). The pH of the aqueous phase was adjusted to 3 with HCl (1 M). The aqueous phase was re-extracted with EA (10 V×3), dried over anhydrous Na₂SO₄, filered, and concentrated under reduced pressure. Heptane (10 v) was added to the resulting solid and the mixture was stirred overnight at room temperature. The precipitated solids were collected by vacuum filtration and were rinsed with heptane (2 V) to afford 4-(ethanesulfonyl)-2-[4-methyl-4-(prop-2-en-1-yloxy) piperidin-1-yl]benzoic acid (77 g, 90% yield).

### Step 6: Synthesis of N-[5-bromo-6-(prop-2-en-1-ylamino)pyridin-2-yl]-4-(ethanesulfonyl)-2-[4-methyl-4-(prop-2-en-1-yloxy)piperidin-1-yl]benzamide

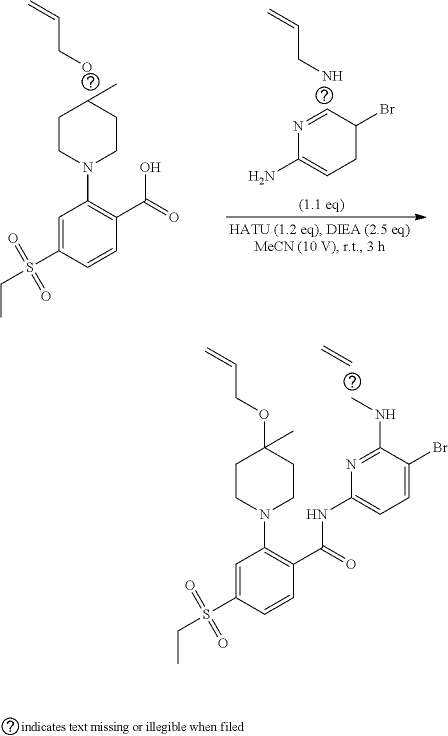

A 250 mL 3-necked round-bottom flask was charged with 4-(ethanesulfonyl)-2-[4-methyl-4-(prop-2-en-1-yloxy)piperidin-1-yl]benzoic acid (15.0 g, 40.8 mmol, 1 equiv) and MeCN (150 mL) at room temperature. To the above mixture was added 3-bromo-N²-(prop-2-en-1-yl)pyridine-2,6-diamine (10.2 g, 44.9 mmol, 1.1 equiv) and HATU (18.6 g, 49.0 mmol, 1.2 equiv) at room temperature. The resulting mixture was stirred at room temperature for 10 min. To the above mixture was added DIEA (13.2 g, 102 mmol, 2.5 equiv) dropwise at 0° C. The resulting mixture was stirred at room temperature for additional 3 h. The resulting mixture was concentrated under reduced pressure. The residue was purified by silica gel column chromatography, eluted with PE/EA (10:1) to afford N-[5-bromo-6-(prop-2-en-1-ylamino)pyridin-2-yl]-4-(ethanesulfonyl)-2-[4-methyl-4-(prop-2-en-1-yloxy)piperidin-1-yl]benzamide (25.2 g, 96% yield).

### Step 7: 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one

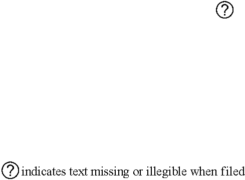

A 5 L 4-necked round-bottom flask was charged with N-[5-bromo-6-(prop-2-en-1-ylamino)pyridin-2-yl]-4-(ethanesulfonyl)-2-[4-methyl-4-(prop-2-en-1-yloxy)piperidin-1-yl]benzamide (25.0 g, 43.3 mmol, 1 equiv) and toluene (3.75 L) at room temperature. [1,3-bis(2,4,6-trimethylphenyl)imidazolidin-2-ylidene]dichloro(phenylmethylidene)ruthenium; tricyclohexylphosphane (1.84 g, 2.16 mmol, 0.05 equiv) in toluene (125 mL) was added dropwise at 60° C. under nitrogen atmosphere. The resulting mixture was stirred at 60° C. overnight under nitrogen atmosphere. The mixture was allowed to cool to room temperature and triethyl phosphite (8.63 g, 51.9 mmol, 1.2 equiv) was added to quench the reaction. The resulting mixture was stirred at room temperature for additional 3 h. The resulting mixture was concentrated under reduced pressure and the resulting residue was purified by silica gel column chromatography (PE/EA 1:1) to afford 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (8.40 g, 35.3% yield).

Step 8: Compounds 301 and 302: 5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (8.10 g, 14.7 mmol, 1 equiv) was purified by reversed-phase flash chromatography with the following conditions: column, C18 silica gel; mobile phase, MeCN in Water (0.1% NH₃·H₂O), 70% to 75% gradient in 20 min; detector, UV 254 nm. The resulting solution was concentrated under vacuum. The resulting mixture was dried by lyophilization with acetonitrile and water overnight to afford (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (3.5863 g, 44.2% yield) and (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (2.9149 g, 36% yield).

Compound 303 was prepared according to General Procedure 2 using Compound 300 and 4-(1H-pyrazol-4-yl)morpholine to afford (E)-2⁵-(ethylsulfonyl)-5⁵-(4-morpholino-1H-pyrazol-1-yl)-4,6-diaza-5(2,6)-pyridina-1(1,4)-piperidina-2(1,2)-benzenacycloundecaphan-9-en-3-one (1.5 mg, 5.6% yield).

### Compound 304.

### Step 1: methyl 4-ethanesulfonamido-2-[4-(prop-2-en-1-yl)piperidin-1-yl]benzoate

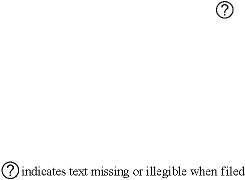

A solution of CuI (17.80 g, 93.4 mmol, 2 equiv) in dioxane (270 mL) was treated with (1R,2R)-1-N,2-N-dimethylcyclohexane-1,2-diamine (13.29 g, 93.4 mmol, 2 equiv) at room temperature for 1 h under nitrogen atmosphere, followed by the addition of methyl 4-iodo-2-[4-(prop-2-en-1-yl)piperidin-1-yl]benzoate (18 g, 46.7 mmol, 1 equiv), K₃PO₄ (29.75 g, 140 mmol, 3 equiv) and ethanesulfonamide (15.30 g, 140 mmol, 3 equiv) at room temperature. The reactor was flushed with nitrogen gas and the reaction mixture was stirred at 50° C. overnight. The mixture was cooled down to 20-30° C. Concentrated to 1-2 V under vacuum below 40° C. To the mixture were charged EA (10 V), H₂O (5 V), sodium citrate (2.0 eq) and sodium bromate (1.5 eq), and stirred for 1-2 h at 20-30° C. Filtered and washed the cake with EA (1 V). The filtrate was extracted and divided. The organic phase was washed with sodium citrate (10 V, 10% w/w). Then the organic phase was washed with aq. NH₄Cl (30 V, saturated). Finally, the organic phase was washed with H₂O (10 V). Concentrated to 1-2 V under vacuum below 40° C. To the resulting mixture was added heptane (15 V) dropwise at 20-30° C. The mixture was stirred for 2-3 h at 20-30° C. and filtered. Then the cake was washed with heptane (2 V). Dried the wet cake at 45-55° C. for 8-12 h. This resulted in methyl 4-ethanesulfonamido-2-[4-(prop-2-en-1-yl)piperidin-1-yl]benzoate (13 g, 76% yield).

### Step 2: 4-ethanesulfonamido-2-[4-(prop-2-en-1-yl)piperidin-1-yl]benzoic acid

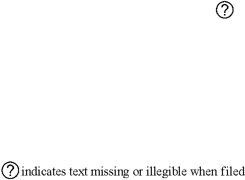

A solution of methyl 4-ethanesulfonamido-2-[4-(prop-2-en-1-yl)piperidin-1-yl]benzoate (13 g, 35.5 mmol, 1 equiv) NaOH (4.26 g, 106 mmol, 3 equiv) in MeOH (78 mL), THF (52 mL), and water (26 mL) was stirred overnight at 50° C. under a nitrogen atmosphere. Then, the mixture was allowed to cool to room temperature, and the reactor was charged with water (3 V). Aqueous HCl (2 M) was added to adjust the pH to 3-4, EA (10 V) was added, and the mixture was extracted 2×. The organic phases were combined, washed with 10% saturated salt water (10 V), and the organic phase was dried by adding anhydrous magnesium sulfate (0.3 w/w), the mixture was filtered, and the filtrate was further concentrated 2-3 V. EA (5 V) was added to the mixture and was removed under reduced pressure (2×), then n-heptane (5 V) was added. After stirring for 2 h, the resulting precipitate was filtered and was washed with n-heptane (2 V) to afford 4-ethanesulfonamido-2-[4-(prop-2-en-1-yl)piperidin-1-yl]benzoic acid (12 g, crude product).

### Step 3: N-[5-bromo-6-(but-3-en-1-ylamino)pyridin-2-yl]-4-ethanesulfonamido-2-[4-(prop-2-en-1-yl)piperidin-1-yl]benzamide

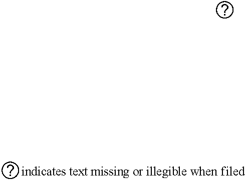

To a stirred solution of 4-ethanesulfonamido-2-[4-(prop-2-en-1-yl)piperidin-1-yl]benzoic acid (12 g, 34.0 mmol, 1.0 equiv) and 3-bromo-N²-(but-3-en-1-yl)pyridine-2,6-diamine (8.24 g, 34.0 mmol, 1 equiv) in DMF (240 mL) were added DIEA (17.60 g, 136 mmol, 4 equiv), PyBOP (26.53 g, 51 mmol, 1.5 equiv) and DMAP (1.25 g, 10.2 mmol, 0.3 equiv) in portions at room temperature under nitrogen atmosphere. The resulting mixture was stirred at 40° C. for 24 h. The resulting mixture was extracted with EtOAc (3×120 mL). The combined organic layers were washed with NaCl (saturated, aq, 2×100 mL), dried over anhydrous Na₂SO₄, and filtered. The filtrate was concentrated under reduced pressure and the resulting residue was purified by silica gel column chromatography (PE/EA 1:1) to afford N-[5-bromo-6-(but-3-en-1-ylamino)pyridin-2-yl]-4-ethanesulfonamido-2-[4-(prop-2-en-1-yl)piperidin-1-yl]benzamide (11 g, 56% yield).

### Step 4: N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide

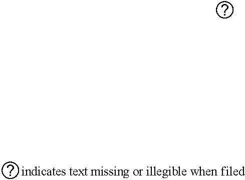

A solution of N-[5-bromo-6-(but-3-en-1-ylamino)pyridin-2-yl]-4-ethanesulfonamido-2-[4-(prop-2-en-1-yl)piperidin-1-yl]benzamide (11 g, 19 mmol, 1 equiv) and Grubbs II catalyst (0.81 g, 0.954 mmol, 0.05 equiv) in toluene (165 mL) was stirred overnight at 60° C. under a nitrogen atmosphere. The mixture was then cooled to room temperature, concentrated under reduced pressure, and the resulting residue was purified by silica gel column chromatography (PE/EA 1:1) to afford N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (7.3 g, 70% yield).

### Step 5: (E) and (Z)—N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide

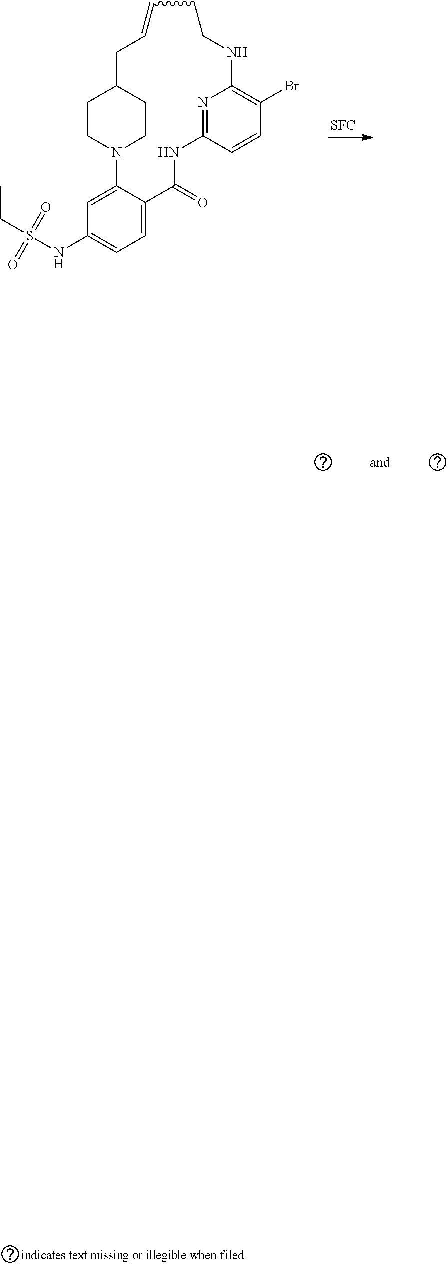

The mixture of cis- and trans-isomers were purified via preparative SFC: (Column: NN-XSELECT HSS PFP, 3.0*100 mm, 3.5 um; Mobile Phase A: CO₂, Mobile Phase B: DCM (0.1% 2M NH₃-MeOH)--HPLC; Flow rate: 100 mL/min; Gradient (B %): isocratic 60% B; Back Pressure(bar): 100; Wave Length: 220 nm; RT1 (min): 4.88; Total Elution Time (min): 11.5; Sample Solvent: DCM: MEOH=4:1(0.2% TEA); Injection Volume: 4.49 mL) to afford (E)-N-(55-bromo-3-oxo-4,6-diaza-5(2,6)-pyridina-1(1,4)-piperidina-2(1,2)-benzenacycloundecaphan-9-en-25-yl)ethanesulfonamide (3.7 g, 49% yield) and (Z)—N-(55-bromo-3-oxo-4,6-diaza-5(2,6)-pyridina-1(1,4)-piperidina-2(1,2)-benzenacycloundecaphan-9-en-25-yl)ethanesulfonamide (1.11 g, 15% yield).

Compound 305 was prepared according to General Procedure 2 using Compound 304 and 2-([3-(trifluoromethyl)-1H-pyrazol-4-yl]oxy)ethan-1-ol to afford (E)-N-(5⁵-(4-(2-hydroxyethoxy)-3-(trifluoromethyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.4 mg, 11.8% yield).

Compound 306 was prepared according to General Procedure 2 using Compound 304 and 3-(methanesulfonylmethyl)-1H-pyrazole to afford (E)-N-(55-(3-((methylsulfonyl)methyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.7 mg, 15.9% yield).

Compound 307 was prepared according to General Procedure 2 using Compound 304 and 4-(cyclopropanesulfonyl)-1H-pyrazole to afford (E)-N-(5⁵-(4-(cyclopropylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.1 mg, 4.1% yield).

Compound 308 was prepared according to General Procedure 2 using Compound 304 and N,N-dimethyl-1H-pyrazole-4-carboxamide to afford (E)-1-(25-(ethylsulfonamido)-3-oxo-4,6-diaza-5(2,6)-pyridina-1(1,4)-piperidina-2(1,2)-benzenacycloundecaphan-9-en-5⁵-yl)-N,N-dimethyl-1H-pyrazole-4-carboxamide (3.9 mg, 17.3% yield).

Compound 309 was prepared according to General Procedure 2 using Compound 304 and 3-fluoro-1H-pyrazole to afford (E)-N-(5⁵-(3-fluoro-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.3 mg, 5.2% yield).

### Compounds 310 and 311.

### Step 1: 4-ethanesulfonamido-2-[4-methyl-4-(prop-2-en-1-yloxy)piperidin-1-yl]benzoate

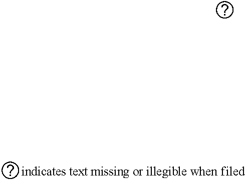

To a solution of rac-(1R,2R)—N1,N2-dimethylcyclohexane-1,2-diamine (13.61 g, 95.8 mmol, 2.0 equiv) in dioxane (300 mL) was added CuI (18.25 g, 95.84 mmol, 2.00 eq.) at 25° C. The reactor was then evacuated under reduced pressure and refilled with nitrogen (3×). The reaction was stirred for 1 h at 25° C. Then, methyl 4-iodo-2-[4-methyl-4-(prop-2-en-1-yloxy)piperidin-1-yl]benzoate (19.90 g, 47.92 mmol, 1.00 eq.), ethanesulfonamide (15.69 g, 144 mmol, 3.00 equiv) and K₃PO₄ (30.5 g, 144 mmol, 3.0 equiv) were added into the reactor. The reaction was stirred for 16 h at 50° C. The mixture was allowed to cool down to room temperature, was concentrated under reduced pressure, and the resulting mixture was diluted with ethyl acetate (200 mL). H₂O (100 mL) sodium citrate (31.08 g, 2.50 equiv) and sodium bromate (9.90 g, 2.00 equiv) were added into reactor and stirred for 2 h at 25° C. The resulting mixture was filtered, and the filter cake was washed with ethyl acetate (50 mL). The filtrate was extracted. The organic phase was washed with 10% w/w sodium citrate (10 V, 200 mL), then washed with aq. NH₄Cl (30 V, 600 mL) and H₂O (10 V, 200 mL). The organic phase was dried over anhydrous Na₂SO₄. After filtration, the filtrate was concentrated under reduced pressure. The residue was purified by trituration with EA: n-heptane (1V:15V=20 mL:300 mL) to afford methyl 4-ethanesulfonamido-2-[4-methyl-4-(prop-2-en-1-yloxy)piperidin-1-yl]benzoate (17.70 g, 93% yield).

### Step 2: 4-ethanesulfonamido-2-[4-methyl-4-(prop-2-en-1-yloxy)piperidin-1-yl]benzoic

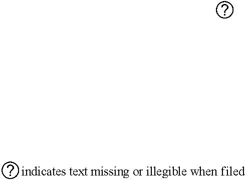

A solution of methyl 4-ethanesulfonamido-2-[4-methyl-4-(prop-2-en-1-yloxy)piperidin-1-yl]benzoate (17.70 g, 44.64 mmol, 1.00 eq.) and NaOH (5.36 g, 133.92 mmol, 3.00 eq.) in MeOH (106.20 mL, 6 V), THF (70.80 mL, 4 V) and H₂O (35.40 mL, 2 V) was stirred at 70° C. for 16 h under nitrogen atmosphere. The mixture was allowed to cool to room temperature and the mixture was concentrated under reduced pressure. The resulting mixture was diluted with THF (90 mL) and H₂O (50 mL). The solution was acidified to pH 3 with 2M HCl (aq.) and was extracted with EtOAc (2×90 mL). The combined organic layers were washed with brine (90 mL), dried over anhydrous Na₂SO₄, filtered, and concentrated under reduced pressure. The resulting residue was purified by trituration with EA: n-heptane (2 V:5 V=36 mL: 90 mL). This resulted in 4-ethanesulfonamido-2-[4-methyl-4-(prop-2-en-1-yloxy)piperidin-1-yl]benzoic acid (16.0 g, 94% yield).

### Step 3: N-[5-bromo-6-(prop-2-en-1-ylamino)pyridin-2-yl]-4-ethanesulfonamido-2-[4-methyl-4-(prop-2-en-1-yloxy)piperidin-1-yl]benzamide

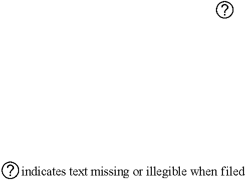

To a stirred solution of 4-ethanesulfonamido-2-[4-methyl-4-(prop-2-en-1-yloxy)piperidin-1-yl]benzoic acid (16.00 g, 41.83 mmol, 1.00 eq.) and 3-bromo-N²-(prop-2-en-1-yl)pyridine-2,6-diamine (10.50 g, 46.01 mmol, 1.10 eq.) in MeCN (160 mL) were added 1-methyl-1H-imidazole (13.74 g, 167.32 mmol, 4.00 eq.) and TCFH (17.61 g, 62.75 mmol, 1.50 eq.) in portions at room temperature under nitrogen atmosphere. The resulting mixture was stirred at 25° C. for 16 h under nitrogen atmosphere. The residue was washed with brine (2×90 mL). The organic layer was dried over anhydrous Na₂SO₄. After filtration, the filtrate was concentrated under reduced pressure. The residue was purified by silica gel column chromatography, eluted with PE:EA=5:1. This resulted in N-[5-bromo-6-(prop-2-en-1-ylamino)pyridin-2-yl]-4-ethanesulfonamido-2-[4-methyl-4-(prop-2-en-1-yloxy)piperidin-1-yl]benzamide (21.40 g, 86% yield).

### Step 4: N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide

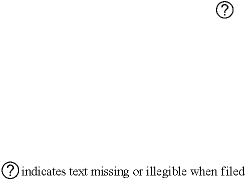

A solution of N-[5-bromo-6-(prop-2-en-1-ylamino)pyridin-2-yl]-4-ethanesulfonamido-2-[4-methyl-4-(prop-2-en-1-yloxy)piperidin-1-yl]benzamide (21.04 g, 36.11 mmol, 1.00 eq.) in toluene (3.3 L, 155 V) was treated with Grubbs' 2^{nd} Generation (1.53 g, 1.806 mmol, 0.05 eq.) at 25° C. under N₂ atmosphere. The reaction was stirred for 18 h at 60° C. The mixture was allowed to cool down to room temperature. The resulting mixture was added Imidazole (2.95 g, 43.34 mmol, 1.20 eq.). The resulting mixture was stirred at room temperature for 2 h under nitrogen atmosphere. The resulting mixture was concentrated under reduced pressure. The residue was purified by silica gel column chromatography, eluted with PE/EA 1:1. This resulted in N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (10.90 g, 53.47% yield).

Step 5: Compounds 310 and 311: 10.90 g of N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide was separated by Prep-SFC with the following conditions (Column: NN-XSELECT HSS PFP, 3.0*100 mm, 3.5 um; Mobile Phase A: CO₂, Mobile Phase B: ACN:MeOH=1:1(1% 2M NH₃-MeOH); Flow rate: 100 mL/min; Gradient (B %): isocratic 40% B; Back Pressure(bar): 100; Wave Length: 220 nm; RT1 (min): 10.73; RT2 (min): 14.83; Total Elution Time (min): 17; Sample Solvent: DCM (0.1% 2M NH₃-MeOH)--HPLC). This resulted in (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (Compound 311) (3.80 g, 34.85% yield) and: (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (Compound 310) (5.60 g, 51.37% yield).

Compound 312 was prepared according to General Procedure 2 using Compound 310 and 3-fluoro-1H-pyrazole to afford (Z)—N-(5⁵-(3-fluoro-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.6 mg, 21.7% yield).

Compound 313 was prepared according to General Procedure 2 using Compound 310 and N,N-dimethyl-1H-pyrazole-4-carboxamide to afford (Z)-1-(25-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-N,N-dimethyl-1H-pyrazole-4-carboxamide (2.9 mg, 10.7% yield).

Compound 314 was prepared according to General Procedure 2 using Compound 310 and 4-(1H-pyrazol-4-ylcarbonyl)morpholine to afford (Z)—N-(1⁴-methyl-5⁵-(4-(morpholine-4-carbonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (2.5 mg, 8.6% yield)

Compound 315 was prepared according to General Procedure 2 using Compound 311 and 4-(1H-pyrazol-4-ylcarbonyl)morpholine to afford (E)-N-(1⁴-methyl-5⁵-(4-(morpholine-4-carbonyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (2.2 mg, 7.5% yield).

Compound 316 was prepared according to General Procedure 2 using Compound 310 and 3-cyclopropyl-1H-pyrazole to afford. (Z)—N-(5⁵-(3-cyclopropyl-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.4 mg, 4.9% yield).

Compound 317 was prepared according to General Procedure 2 using Compound 311 and 3-cyclopropyl-1H-pyrazole to afford (E)-N-(55-(3-cyclopropyl-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.2 mg, 9.1% yield).

Compound 318 was prepared according to General Procedure 2 using Compound 304 and 3-(2,2,2-trifluoroethyl)-1H-pyrazole to afford (E)-N-(3-oxo-5⁵-(3-(2,2,2-trifluoroethyl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (1.7 mg, 5.9% yield).

Compound 319 was prepared according to General Procedure 2 using Compound 304 and 3-cyclopropyl-1H-pyrazole to afford (E)-N-(55-(3-cyclopropyl-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (8.9 mg, 32.1% yield).

Compound 320 was prepared according to General Procedure 2 using Compound 311 and N-cyclopentyl-1H-pyrazol-4-amine to afford (E)-N-(5⁵-(4-(cyclopentylamino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.8 mg, 2.8% yield).

Compound 321 was prepared according to General Procedure 2 using Compound 311 and N-[(1H-pyrazol-3-yl)methyl]acetamide to afford (E)-N-((1-(2⁵-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-3-yl)methyl)acetamide (1.6 mg, 6.5% yield).

Compound 322 was prepared according to General Procedure 2 using Compound 310 and 2-(1H-pyrazol-4-yl)piperidine to afford (Z)—N-(1⁴-methyl-3-oxo-5⁵-(4-(piperidin-2-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (11.5 mg, 38.4% yield).

Compound 323 was prepared according to General Procedure 2 using Compound 304 and N-cyclopentyl-1H-pyrazol-4-amine to afford (E)-N-(5⁵-(4-(cyclopentylamino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.9 mg, 16.7% yield).

Compound 324 was prepared according to General Procedure 2 using Compound 310 and N-[(1H-pyrazol-3-yl)methyl]acetamide to afford (Z)—N-((1-(25-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-3-yl)methyl)acetamide (1.0 mg, 4.0% yield).

Compound 325 was prepared according to General Procedure 2 using Compound 304 and 2-(1H-pyrazol-4-yl)piperidine to afford (E)-N-(3-oxo-5⁵-(4-(piperidin-2-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2.8 mg, 9.6% yield).

Compound 326 was prepared according to General Procedure 2 using Compound 310 and N-cyclopentyl-1H-pyrazol-4-amine to afford (Z)—N-(5⁵-(4-(cyclopentylamino)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.1 mg, 13.8% yield).

Compound 327 was prepared according to General Procedure 2 using Compound 304 and N-[(1H-pyrazol-3-yl)methyl]acetamide to afford (E)-N-((1-(2⁵-(ethylsulfonamido)-3-oxo-4,6-diaza-5(2,6)-pyridina-1(1,4)-piperidina-2(1,2)-benzenacycloundecaphan-9-en-5⁵-yl)-1H-pyrazol-3-yl)methyl)acetamide (1.5 mg, 6% yield).

Compound 328 was prepared according to General Procedure 1 using Compound 299 and N-methyl methanesulfonamide to afford (E)-N-methyl-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (11.1 mg, 44.8% yield).

Compound 329 was prepared according to General Procedure 1 using Compound 299 and 2,2,2-trifluoroethane-1-sulfonamide to afford (E)-2,2,2-trifluoro-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (3.1 mg, 11.3% yield).

Compound 330 was prepared according to General Procedure 1 using Compound 299 and oxetane-3-sulfonamide to afford (E)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)oxetane-3-sulfonamide (3 mg, 10% yield).

Compound 331 was prepared according to General Procedure 1 using Compound 299 and (2R)-1-hydroxypropane-2-sulfonamide to afford (R,E)-1-hydroxy-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)propane-2-sulfonamide (11.3 mg, 37% yield).

Compound 332 was prepared according to General Procedure 1 using Compound 299 and N-methylethane-1-sulfonamide to afford (E)-N-methyl-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (12.7 mg, 42.3% yield).

Compound 333 was prepared according to General Procedure 1 using Compound 299 and 1,3,4-oxathiazinane 3,3-dioxide to afford (E)-2⁵-(3,3-dioxido-1,3,4-oxathiazinan-4-yl)-5⁵-(4,-morpholino-1H-pyrazol-1-yl)-4,6-diaza-5(2,6)-pyridina-1(1,4)-piperidina-2(1,2)-benzenacycloundecaphan-9-en-3-one (15.3 mg, 50.2% yield).

Compound 334 was prepared according to General Procedure 1 using Compound 299 and (2S)-1-hydroxypropane-2-sulfonamide to afford (S,E)-1-hydroxy-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)propane-2-sulfonamide (6.8 mg, 22.2% yield).

Compound 335 was prepared according to General Procedure 1 using Compound 299 and difluoromethanesulfonamide to afford (E)-1,1-difluoro-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (6.6 mg, 21.8% yield).

Compound 336 was prepared according to General Procedure 1 using Compound 299 and 2-(dimethylamino)ethane-1-sulfonamide to afford (E)-2-(dimethylamino)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-25-yl)ethane-1-sulfonamide (11.7 mg, 43.9% yield).

Compound 337 was prepared according to General Procedure 1 using Compound 299 and 2-hydroxy-N-methylethane-1-sulfonamide to afford (E)-2-hydroxy-N-methyl-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-25-yl)ethane-1-sulfonamide (12.2 mg, 45.6% yield).

Compound 338 was prepared according to General Procedure 1 using Compound 299 and 1-(hydroxymethyl)cyclopropane-1-sulfonamide to afford (E)-1-(hydroxymethyl)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)cyclopropane-1-sulfonamide (2.1 mg, 6.8% yield).

Compound 339 was prepared according to General Procedure 1 using Compound 299 and 2-hydroxyethane-1-sulfonamide to afford (E)-2-hydroxy-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (10.1 mg, 33.8% yield).

Compound 340 was prepared according to General Procedure 1 using Compound 299 and isothiazolidine 1,1-dioxide to afford (E)-2⁵-(1,1-dioxidoisothiazolidin-2-yl)-5⁵-(4-morpholino-1H-pyrazol-1-yl)-4,6-diaza-5(2,6)-pyridina-1(1,4)-piperidina-2(1,2)-benzenacycloundecaphan-9-en-3-one (13.2 mg, 50.9% yield).

Compound 341 was prepared according to General Procedure 1 using Compound 299 and methanesulfonamide to afford (E)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (1.6 mg, 6.4% yield).

Compound 342 was prepared according to General Procedure 1 using Compound 299 and 2-methoxyethane-1-sulfonamide to afford (E)-2-methoxy-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)ethane-1-sulfonamide (13.3 mg, 49.8% yield).

Compound 343 was prepared according to General Procedure 1 using Compound 299 and 2-oxazolidone to afford (E)-3-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)oxazolidin-2-one (4.1 mg, 14.5% yield).

Compound 344 was prepared according to General Procedure 1 using Compound 299 and 1,4-butanesultam to afford (E)-2⁵-(1,1-dioxido-1,2-thiazinan-2-yl)-5⁵-(4-morpholino-1H-pyrazol-1-yl)-4,6-diaza-5(2,6)-pyridina-1(1,4)-piperidina-2(1,2)-benzenacycloundecaphan-9-en-3-one (10.5 mg, 34.6% yield).

Compound 345 was prepared according to General Procedure 1 using Compound 299 and 2-methyl-1,2,5-thiadiazolidine 1,1-dioxide to afford (E)-2⁵-(5-methyl-1,1-dioxido-1,2,5-thiadiazolidin-2-yl)-5⁵-(4-morpholino-1H-pyrazol-1-yl)-4,6-diaza-5(2,6)-pyridina-1(1,4)-piperidina-2(1,2)-benzenacycloundecaphan-9-en-3-one (3.2 mg, 12% yield).

Compound 346 was prepared according to General Procedure 1 using Compound 299 and 2-imidazolidone to afford (E)-5⁵-(4-morpholino-1H-pyrazol-1-yl)-2⁵-(2-oxoimidazolidin-1-yl)-4,6-diaza-5(2,6)-pyridina-1(1,4)-piperidina-2(1,2)-benzenacycloundecaphan-9-en-3-one (7.6 mg, 31.8% yield).

Compound 347 was prepared according to General Procedure 1 using Compound 299 and N-methylmethanesulfonoimidamide hydrochloride to afford (E)-N-methyl-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-25-yl)methanesulfonimidamide (0.4 mg, 1.2% yield).

Compound 348 was prepared according to General Procedure 1 using Compound 299 and 1-methylcyclopropane-1-sulfonamide to afford (E)-1-methyl-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)cyclopropane-1-sulfonamide (11.3 mg, 43.8% yield).

Compound 349 was prepared according to General Procedure 1 using Compound 299 and N-(2-hydroxyethyl)methanesulfonamide to afford (E)-N-(2-hydroxyethyl)-N-(5⁵-(4-morpholino-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (4.1 mg, 13.4% yield).

Compound 350 was prepared according to General Procedure 1 using Compound 299 and 1,2,5-thiadiazolidine 1,1-dioxide to afford (E)-2⁵-(1,1-dioxido-1,2,5-thiadiazolidin-2-yl)-5⁵-(4-morpholino-1H-pyrazol-1-yl)-4,6-diaza-5(2,6)-pyridina-1(1,4)-piperidina-2(1,2)-benzenacycloundecaphan-9-en-3-one (5.4 mg, 21.2% yield).

### Compound 351.

### Step 1: 4-(1-((tert-butyldimethylsilyl) oxy) vinyl)-1-((2-(trimethylsilyl) ethoxy) methyl)-1H-pyrazole

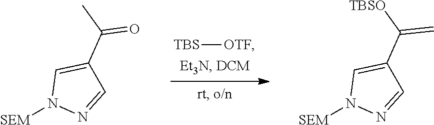

Et₃N (3.0 equiv) was added to the mixture of 1-(1-((2-(trimethylsilyl) ethoxy) methyl)-1H-pyrazol-4-yl) ethan-1-one (1.0 equiv) and TBS-OTF (3.0 equiv) in DCM (10 V). The mixture was stirred at room temperature for 16 hours. The resulting mixture was diluted with water (10 V). The resulting mixture was extracted with DCM (3×10 V). The combined organic layers were washed with brine (20 V), dried over anhydrous Na₂SO₄. After filtration, the filtrate was concentrated under reduced pressure. The residue was purified by reversed-phase flash chromatography with the following conditions: Column, C18 silica gel; mobile phase, aq. 0.04% NH₄HCO₃ in ACN, 10% to 60% gradient in 10 min; detector, UV 254 nm to afford the product 4-(1-((tert-butyldimethylsilyl) oxy) vinyl)-1-((2-(trimethylsilyl) ethoxy) methyl)-1H-pyrazole (68% yield). LCMS (ESI-MS) m/z=354.2 [M+H]⁺.

### Step 2: 4-(1-((tert-butyldimethylsilyl) oxy) cyclopropyl)-1-((2-(trimethylsilyl) ethoxy) methyl)-1H-pyrazole

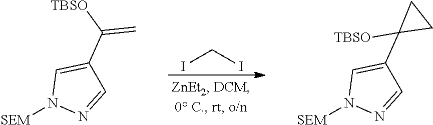

1 M Diethylzinc in n-hexane (10 equiv) was slowly added to 4-(1-((tert-butyldimethylsilyl) oxy) vinyl)-1-((2-(trimethylsilyl) ethoxy) methyl)-1H-pyrazole (1.0 equiv) in DCM (5 V) at 0° C. under a nitrogen atmosphere. The mixture was stirred at 0° C. for 1 hour. Diiodomethane (10 equiv) was added to the reaction mixture at 0° C. slowly and stirred at room temperature for 16 hours. Water (50 V) was added to the mixture, the resulting solution was extracted with DCM (2×500 mL), the organic layer was washed with brine (50 V), dried over anhydrous Na₂SO₄. After filtration, the filtrate was concentrated under reduced pressure. The residue was purified by reversed-phase flash chromatography with the following conditions: column, C18 silica gel; mobile phase, aq. 0.04% NH₄HCO₃ in ACN, 10% to 70% gradient in 10 min; detector, UV 254 nm to afford the product 4-(1-((tert-butyldimethylsilyl) oxy) cyclopropyl)-1-((2-(trimethylsilyl) ethoxy) methyl)-1H-pyrazole (24% yield). LCMS (ESI-MS) m/z=369.2 [M+H]⁺.

### Step 3: 4-(1-((tert-butyldimethylsilyl)oxy)cyclopropyl)-1H-pyrazole

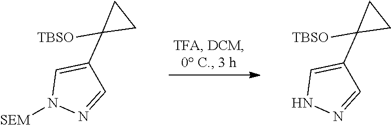 [SMILES: CC(C)(C)[Si](C)(C)OC1(c2cnn(COCC[Si](C)(C)C)c2)CC1 ; CC(C)(C)[Si](C)(C)OC1(c2cn[nH]c2)CC1]

TFA (4 V) was added to 4-(1-((tert-butyldimethylsilyl) oxy) cyclopropyl)-1-((2-(trimethylsilyl) ethoxy) methyl)-1H-pyrazole (1.0 equiv) in DCM (8 V) at 0° C. The mixture was stirred at 0° C. for 3 hours. The mixture was basified to pH 9 with aq. Na₂CO₃. The resulting mixture was extracted with DCM (3×5 V). The combined organic layers were washed with brine (8 V), dried over anhydrous Na₂SO₄. After filtration, the filtrate was concentrated under reduced pressure. The residue was purified by reversed-phase flash chromatography with the following conditions: Column, C18 silica gel; mobile phase, aq. 0.04% NH₄HCO₃ in ACN, 10% to 50% gradient in 10 min; detector, UV 254 nm to afford the product 4-(1-((tert-butyldimethylsilyl)oxy)cyclopropyl)-1H-pyrazole (24% yield).

### Step 4: (E)-5⁵-(4-(1-((tert-butyldimethylsilyl) oxy) cyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one

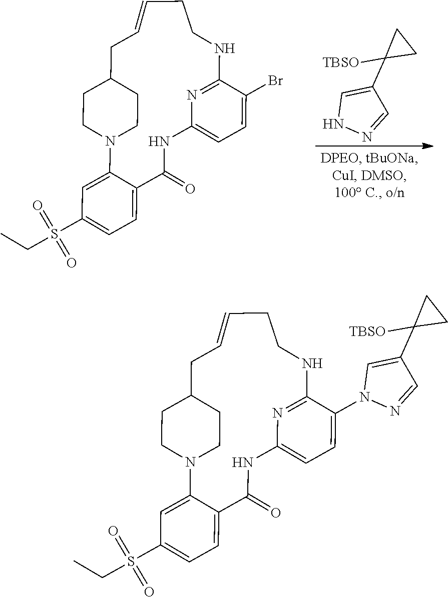 [SMILES: CCS(=O)(=O)c1ccc2c(c1)N1CCC(C/C=C/CCNc3nc(ccc3Br)NC2=O)CC1 ; CC(C)(C)[Si](C)(C)OC1(c2cn[nH]c2)CC1 ; CCS(=O)(=O)c1ccc2c(c1)N1CCC(C/C=C/CCNc3nc(ccc3-n3cc(C4(O[Si](C)(C)C(C)(C)C)CC4)cn3)NC2=O)CC1]

t-BuONa (4.0 equiv.) was added to the mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (1.0 equiv, Compound 300), 4-(1-((tert-butyldimethylsilyl)oxy)cyclopropyl)-1H-pyrazole (1.8 equiv.), (N, N-Bis(2-phenylethyl)oxamide (0.1 equiv), CuI (0.1 equiv) in DMSO (7 V) under nitrogen atmosphere. The mixture was stirred at 100° C. overnight. After cooling to room temperature, the mixture was purified by reversed-phase flash chromatography with the following conditions: Column, C18 silica gel; mobile phase, aq. 0.1% NH₄HCO₃ in ACN, 10% to 100% gradient in 10 min; detector, UV 254 nm to afford the product (E)-5⁵-(4-(1-((tert-butyldimethylsilyl)oxy)cyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (45% yield). LCMS (ESI-MS) m/z=691.3 [M+H]⁺.

Step 5: Compound 351: 1 M TBAF in THF (2.0 equiv) was added to the mixture of (E)-5⁵-(4-(1-((tert-butyldimethylsilyl) oxy) cyclopropyl)-1H-pyrazol-1-yl)-2⁵-(ethylsulfonyl)-4,6-diaza-5(2,6)-pyridina-1(1,4)-piperidina-2(1,2)-benzen-acycloundecaphan-9-en-3-one (1.0 equiv) in THF (12 V). The mixture was stirred at room temperature for 5 hours. The mixture was concentrated under vacuum to afford the residue mixture. The residue mixture was purified by reversed-phase flash chromatography with the following conditions: Column, C18 silica gel; mobile phase, aq. 0.04% NH₄HCO₃ in ACN, 10% to 35% gradient in 10 min; detector, UV 254 nm to afford the crude product (80% purity). The crude product mixture was purified by silica gel column chromatography, eluted with EA:PE (1:3) to afford (E)-2⁵-(ethylsulfonyl)-5⁵-(4-propionyl-1H-pyrazol-1-yl)-4,6-diaza-5(2,6)-pyridina-1(1,4)-piperidina-2(1,2)-benzenacycloundecaphan-9-en-3-one (54% yield).

### Compound 352.

### Step 1: (Z)—N-(5⁵-(4-(1-((tert-butyldimethylsilyl) oxy) cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide

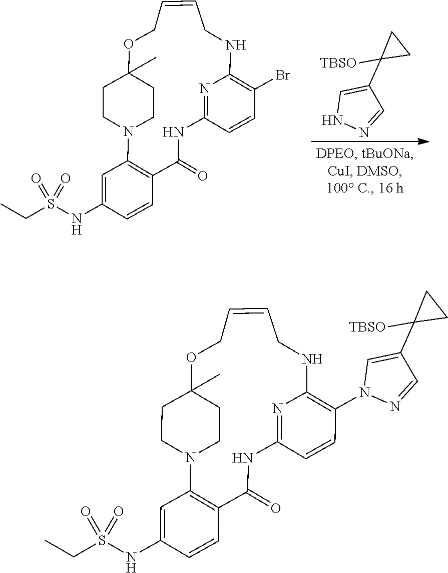 [SMILES: CCS(=O)(=O)Nc1ccc2c(c1)N1CCC(C)(CC1)OC/C=C\CNc1nc(ccc1Br)NC2=O ; CC(C)(C)[Si](C)(C)OC1(c2cn[nH]c2)CC1 ; CCS(=O)(=O)Nc1ccc2c(c1)N1CCC(C)(CC1)OC/C=C\CNc1nc(ccc1-n1cc(C3(O[Si](C)(C)C(C)(C)C)CC3)cn1)NC2=O]

t-BuONa (3.0 equiv) was added to the mixture of ((Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.0 equiv), 4-(1-((tert-butyldimethylsilyl)oxy)cyclopropyl)-1H-pyrazole (20 equiv), CuI (1.0 equiv) and N¹,N²-diphenethyloxalamide (1.0 equiv) in DMSO (20 V) under nitrogen atmosphere. The mixture was stirred at 100° C. overnight. After cooling to room temperature, the reaction mixture was diluted with H₂O (50 V) and extracted with EA (3×50 V). The combined organic layers were washed with brine (150 V), dried over anhydrous Na₂SO₄, filtered and concentrated under vacuum to afford the crude product. The crude product purified by reversed-phase flash chromatography, with the following conditions: column, C18 silica gel; mobile phase, aq. 0.1% NH₄HCO₃ in [ACN], 10% to 100% gradient in 10 min; detector, UV 254 nm to afford (Z)—N-(5⁵-(4-(1-((tert-butyldimethylsilyl)oxy)cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (39% yield). LCMS (ESI-MS) m/z=722.0 [M+H]⁺.

Step 2: Compound 352: 1M TBAF in THF (2.0 equiv) was added to (Z)—N-(5⁵-(4-(1-((tert-butyldimethylsilyl) oxy) cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (1.0 equiv) in THF (20 V). The mixture was stirred at room temperature for 2 hours. The reaction was poured into aq. NH₄Cl (20 V) at temperature. The resulting mixture was extracted with EA (20 V). The combined organic layers were washed with aq. NH₄Cl (2×20 V), dried over anhydrous Na₂SO₄. After filtration, the filtrate was concentrated under reduced pressure. The crude product purified by silica gel column chromatography, eluted with DCM:MeOH (20:1) to afford (Z)—N-(5⁵-(4-(1-hydroxycyclopropyl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (40% yield).

### Compound 353.

### Step 1: (E)-N-(5⁵-(4-(1-((tert-butyldimethylsilyl) oxy) cyclopropyl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl) ethanesulfonamide

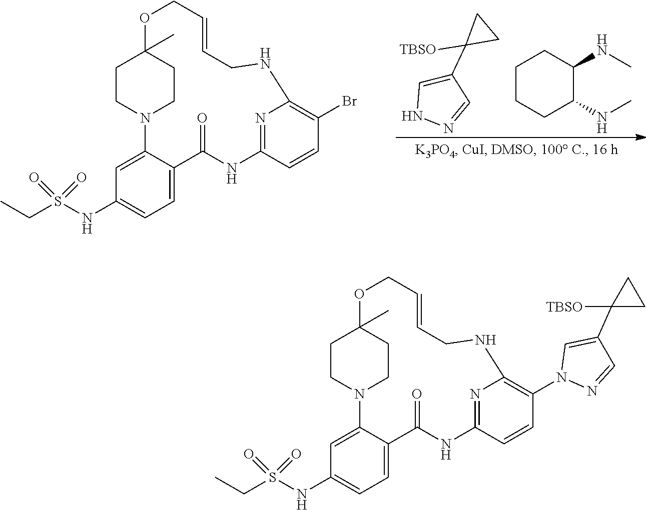

K₃PO₄ (56 mg, 0.27 mmol) was added to the mixture of (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (50 mg, 0.09 mmol), 4-(1-((tert-butyldimethylsilyl)oxy)cyclopropyl)-1H-pyrazole (100 mg, 0.41 mmol), CuI (2 mg, 0.01 mmol) and (1R,2R)—N¹,N²-dimethylcyclohexane-1,2-diamine (2 mg, 0.01 mmol) in DMSO (5 mL) under nitrogen atmosphere. The mixture was stirred at 100° C. overnight. After cooling to room temperature, the reaction mixture was diluted with H₂O (50 mL) and extracted with EA (3×50 mL). The combined organic layers were washed with brine (200 mL), dried over anhydrous Na₂SO₄, filtered and concentrated under vacuum to afford the crude product. The crude product purified by reversed-phase flash chromatography, with the following conditions: Column, C₁₈ silica gel; mobile phase, aq. 0.1% NH₄HCO₃ in ACN, 10% to 100% gradient in 10 min; detector, UV 254 nm to afford (E)-N-(5⁵-(4-(1-((tert-butyldimethylsilyl)oxy)cyclopropyl)-1H-pyrazol-1-yl)-1⁴-methyl-3-oxo-11-oxa-4,6-diaza-5(2,6)-pyridina-1(1,4)-piperidina-2(1,2)-benzena-cycloundecaphan-8-en-2⁵-yl)ethanesulfonamide (30 mg, 44% yield). LCMS (ESI-MS) m/z=722.0 [M+H]⁺.

Step 2: Compound 353: 1M TBAF in THF (0.09 mL, 0.09 mmol) was added to (E)-N-(5⁵-(4-(1-((tert-butyldimethylsilyl) oxy) cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (25 mg, 0.04 mmol) in THF (10 mL). The mixture was stirred at room temperature for 1 hour. The reaction was poured into aq. NH₄Cl (20 mL) at temperature. The resulting mixture was extracted with EA (20 mL). The combined organic layers were washed with aq. NH₄Cl (2×20 mL), dried over anhydrous Na₂SO₄. After filtration, the filtrate was concentrated under reduced pressure. The crude product purified by silica gel column chromatography, eluted with DCM:MeOH (20:1) to afford (E)-N-(5⁵-(4-(1-hydroxycyclopropyl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (13.4 mg, 60% yield).

### Compounds 356 and 357.

### Step 1A: tert-butyl (6-chloro-5-iodopyridin-2-yl) carbamate

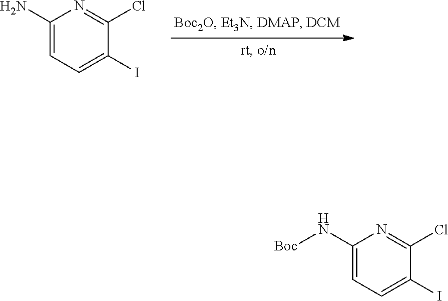 [SMILES: Nc1ccc(I)c(Cl)n1 ; CC(C)(C)OC(=O)Nc1ccc(I)c(Cl)n1]

DMAP (0.2 g, 2 mmol) was added to the mixture of 6-chloro-5-iodopyridin-2-amine (5 g, 0.02 mol), Et₃N (6 g, 60 mmol) and Boc₂O (6 g, 30 mmol) in DCM (50 mL) at 0° C. The mixture was stirred at room temperature overnight. The resulting mixture was diluted with water (100 mL). The resulting mixture was extracted with DCM (2×100 mL). The combined organic layers were washed with brine (400 mL), dried over anhydrous Na₂SO₄. After filtration, the filtrate was concentrated under reduced pressure. The mixture was purified by silica gel column chromatography, eluted with EA:PE (1:5) to afford tert-butyl (6-chloro-5-iodopyridin-2-yl)carbamate (2.4 g, 30% yield). LCMS (ESI-MS) m/z=355.0 [M+H]⁺.

### Step 2A: tert-butyl (6-chloro-5-(4-iodo-1H-pyrazol-1-yl)pyridin-2-yl)carbamate

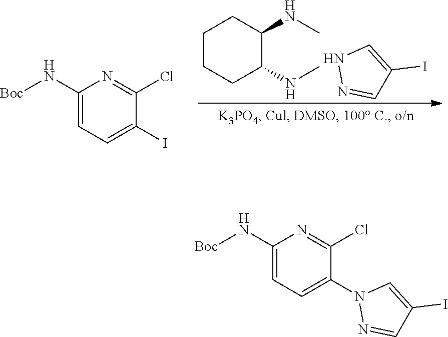

K₃PO₄ (4.8 g, 23 mmol) was added to the mixture of 4-iodo-1H-pyrazole (2.0 g, 10 mmol), tert-butyl (6-chloro-5-iodopyridin-2-yl) carbamate (2.0 g, 5.6 mmol), (1R,2R)—N¹, N²-dimethylcyclohexane-1,2-diamine (80 mg, 0.56 mmol) and CuI (0.11 g, 0.56 mmol) in DMSO (20 mL) under nitrogen atmosphere. The mixture was stirred at 100° C. overnight. The reaction mixture was purified by reversed-phase flash chromatography with the following conditions: column, C18 silica gel; mobile phase, aq. 0.04% TFA in ACN, 10% to 70% gradient in 10 min; detector, UV 254 nm to afford the product tert-butyl (6-chloro-5-(4-iodo-1H-pyrazol-1-yl)pyridin-2-yl)carbamate (1.5 g, 51% yield). LCMS (ESI-MS) m/z=421.1 [M+H]⁺.

### Step 3A: tert-butyl (5-(4-(azetidin-1-yl)-1H-pyrazol-1-yl)-6-chloropyridin-2-yl) carbamate

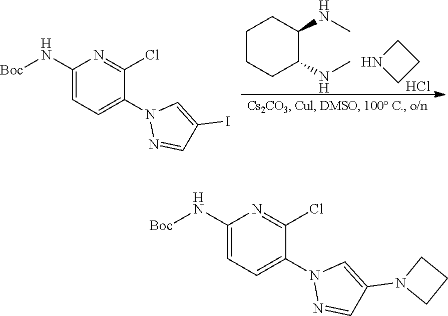

Cs₂CO₃ (3.5 g, 11 mmol) was added to the mixture of tert-butyl (6-chloro-5-(4-iodo-1H-pyrazol-1-yl) pyridin-2-yl) carbamate (1.5 g, 3.6 mmol), (1R,2R)—N¹, N²-dimethylcyclohexane-1,2-diamine (0.51 g, 3.6 mmol), azetidine hydrochloride (1.7 g, 18 mmol) and CuI (0.68 g, 3.6 mmol) in DMSO (20 mL) under nitrogen atmosphere. The mixture was stirred at 100° C. overnight. The reaction mixture was purified by reversed-phase flash chromatography with the following conditions: column, C18 silica gel; mobile phase, aq. 0.04% TFA in ACN, 10% to 60% gradient in 10 min; detector, UV 254 nm to afford the crude product tert-butyl (5-(4-(azetidin-1-yl)-1H-pyrazol-1-yl)-6-chloropyridin-2-yl) carbamate (800 mg, 64% yield). LCMS (ESI-MS) m/z=350.1 [M+H]⁺.

### Step 4A: tert-butyl (6-(allylamino)-5-(4-(azetidin-1-yl)-1H-pyrazol-1-yl) pyridin-2-yl) carbamate

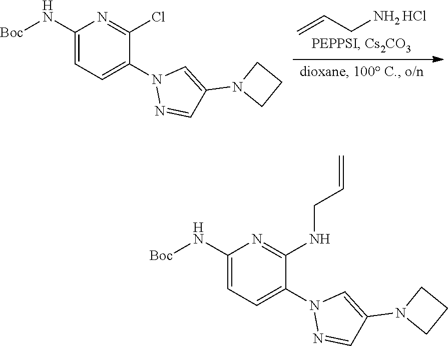

(SP-4-1)-[1,3-Bis[2,6-bis(1-propylbutyl)phenyl]-4,5-dichloro-1,3-dihydro-2H-imidazol-2-ylidene]dichloro(3-chloropyridine-kappaN)-Palladium (195 mg, 0.20 mmol) was added to the mixture of tert-butyl (5-(4-(azetidin-1-yl)-1H-pyrazol-1-yl)-6-chloropyridin-2-yl)carbamate (700 mg, 2.0 mmol), prop-2-en-1-amine hydrochloride (562 mg, 6.0 mmol) and Cs₂CO₃ (1.96 g, 6.0 mmol) in 1,4-dioxane (10 mL) under nitrogen atmosphere. The resulting mixture was filtered, the filter cake was washed with DCM (3×10 mL). The filtrate was concentrated under reduced pressure. The residue was purified by silica gel column chromatography, eluted with EA:PE (1:2) to afford tert-butyl (6-(allylamino)-5-(4-(azetidin-1-yl)-1H-pyrazol-1-yl)pyridin-2-yl)carbamate (300 mg, 40.5% yield). LCMS (ESI-MS) m/z=371.2 [M+H]⁺.

### Step 5A: N²-allyl-3-(4-(azetidin-1-yl)-1H-pyrazol-1-yl)pyridine-2,6-diamine

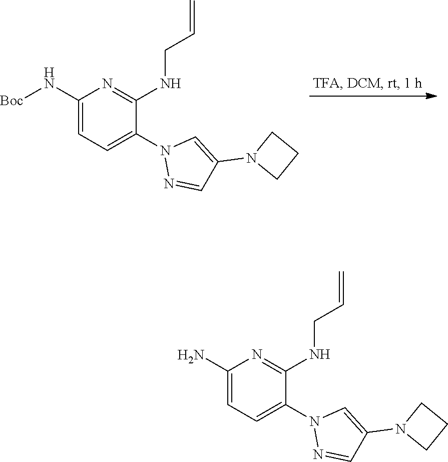 [SMILES: C=CCNc1nc(NC(=O)OC(C)(C)C)ccc1-n1cc(N2CCC2)cn1 ; C=CCNc1nc(N)ccc1-n1cc(N2CCC2)cn1]

TFA (5 mL) was added to the mixture of tert-butyl (6-(allylamino)-5-(4-(azetidin-1-yl)-1H-pyrazol-1-yl) pyridin-2-yl) carbamate (300 mg, 0.81 mmol) in DCM (10 mL). The mixture was stirred at room temperature for 1 hour. The resulting mixture was concentrated under vacuum to afford the crude product N²-allyl-3-(4-(azetidin-1-yl)-1H-pyrazol-1-yl) pyridine-2,6-diamine (265 mg crude). LCMS (ESI-MS) m/z=271.1 [M+H]⁺.

### Step 1B: methyl 2-(4-(allyloxy)-4-methylpiperidin-1-yl)-4-iodobenzoate

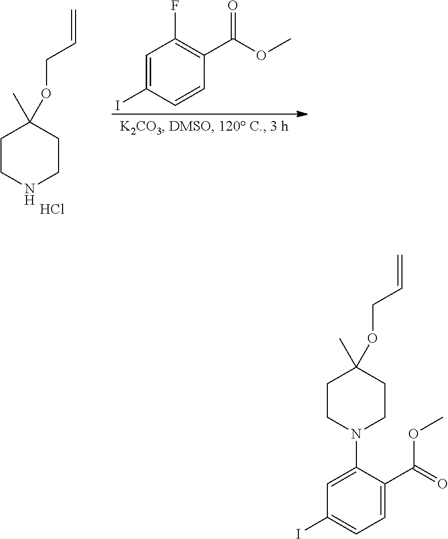 [SMILES: C=CCOC1(C)CCNCC1 ; COC(=O)c1ccc(I)cc1F ; Cl ; C=CCOC1(C)CCN(c2cc(I)ccc2C(=O)OC)CC1]

K₂CO₃ (14.8 g, 107 mmol) was added to a stirred mixture of methyl 2-fluoro-4-iodobenzoate (10 g, 35 mmol) and 4-(allyloxy)-4-methylpiperidine hydrochloride (8.2 g, 42 mmol) in DMSO (100 mL). The resulting mixture was stirred at 120° C. for 3 hours. After cooling to room temperature, the reaction mixture was diluted with H₂O (500 mL) and extracted with EA (3×500 mL). The combined organic layers were washed with brine (2 L), dried over anhydrous Na₂SO₄, filtered and concentrated under vacuum to afford the crude product. The crude product purified by silica gel column chromatography, eluted with PE:EA (3:1) to afford methyl 2-(4-(allyloxy)-4-methylpiperidin-1-yl)-4-iodobenzoate (12 g, 81% yield). LCMS (ESI-MS) m/z=416.1 [M+H]⁺.

### Step 2B: 2-(4-allyloxy)-4-methylpiperidin-1-yl)-4-(ethylsulfonyl) benzoate

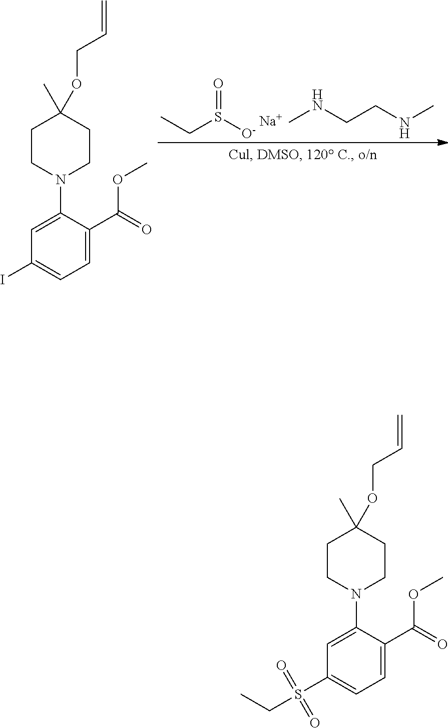

CuI (92 mg, 0.48 mmol) was added to a stirred mixture of methyl 2-(4-(allyloxy)-4-methylpiperidin-1-yl)-4-iodobenzoate (2 g, 4.81 mmol), sodium ethanesulfinate (839 mg, 7.23 mmol) and N¹, N²-dimethylethane-1,2-diamine (43 mg, 0.48 mmol) in DMSO (20 mL). The resulting mixture was stirred at 120° C. overnight. After cooling to room temperature, the reaction mixture was diluted with H₂O (200 mL) and extracted with EA (3×200 mL). The combined organic layers were washed with brine (1 L), dried over anhydrous Na₂SO₄, filtered and concentrated under vacuum to afford the crude product. The crude product purified by silica gel column chromatography, eluted with PE:EA (5:1) to afford methyl 2-(4-(allyloxy)-4-methylpiperidin-1-yl)-4-(ethylsulfonyl) benzoate (1.59 g, 90% yield). LCMS (ESI-MS) m/z=382.2 [M+H]⁺.

### Step 3B: 2-(4-(allyloxy)-4-methylpiperidin-1-yl)-4-(ethylsulfonyl)benzoic acid

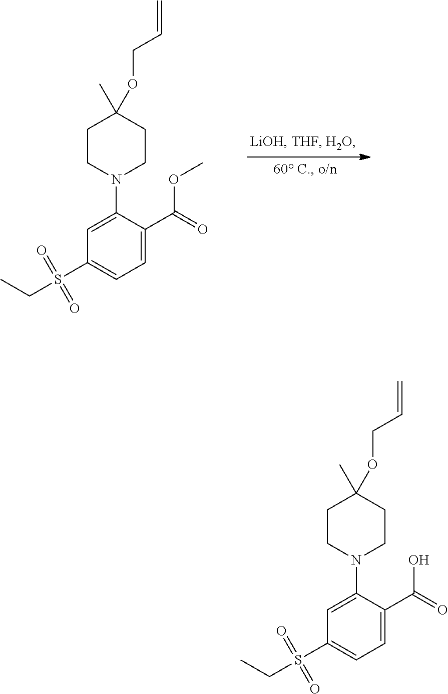 [SMILES: C=CCOC1(C)CCN(c2cc(S(=O)(=O)CC)ccc2C(=O)OC)CC1 ; C=CCOC1(C)CCN(c2cc(S(=O)(=O)CC)ccc2C(=O)O)CC1]

LiOH (600 mg, 25 mmol) was added to a stirred mixture of methyl 2-(4-(allyloxy)-4-methylpiperidin-1-yl)-4-(ethylsulfonyl) benzoate (1.59 g, 4.17 mmol) in THF:H₂O (3:1, 20 mL). The resulting mixture was stirred at 60° C. overnight. The mixture was acidified to pH=2-3 with saturated 6N HCl, then the aqueous layer was extracted with EA (3×20 mL). The combined organic layers were washed with brine (100 mL), dried over anhydrous Na₂SO₄, filtered and concentrated under vacuum to afford 2-(4-(allyloxy)-4-methylpiperidin-1-yl)-4-(ethylsulfonyl) benzoic acid (1.49 g crude). LCMS (ESI-MS) m/z=368.1 [M+H]⁺.

### Step 7. N-(6-(allylamino)-5-(4-(azetidin-1-yl)-1H-pyrazol-1-yl) pyridin-2-yl)-2-(4-(allyloxy)-4-methylpiperidin-1-yl)-4-(ethylsulfonyl) benzamide

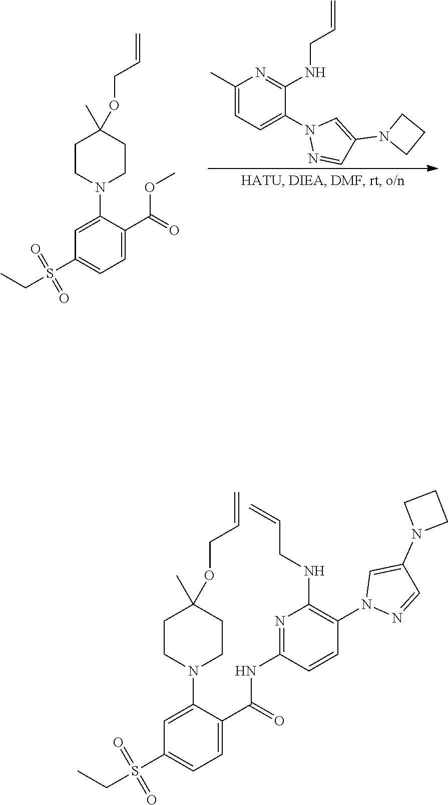 [SMILES: C=CCOC1(C)CCN(c2cc(S(=O)(=O)CC)ccc2C(=O)OC)CC1 ; C=CCNc1nc(C)ccc1-n1cc(N2CCC2)cn1 ; C=CCNc1nc(NC(=O)c2ccc(S(=O)(=O)CC)cc2N2CCC(C)(OCC=C)CC2)ccc1-n1cc(N2CCC2)cn1]

DIEA (280 mg, 2.16 mmol) was added to the mixture of 2-(4-(allyloxy)-4-methylpiperidin-1-yl)-4-(ethylsulfonyl) benzoic acid (265 mg, 0.72 mmol), N²-allyl-3-(4-(azetidin-1-yl)-1H-pyrazol-1-yl) pyridine-2,6-diamine (195 mg, 0.72 nmol) and HATU (411 mg, 1.08 mmol) in DMF (5 mL). The mixture was stirred at room temperature overnight. The reaction mixture was diluted with water (50 mL). The resulting mixture was extracted with EA (3×50 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (1:4) to afford N-(6-(allylamino)-5-(4-(azetidin-1-yl)-1H-pyrazol-1-yl) pyridin-2-yl)-2-(4-(allyloxy)-4-methylpiperidin-1-yl)-4-(ethylsulfonyl) benzamide (250 mg, 45% yield). LCMS (ESI-MS) m/z=620.3 [M+H]⁺.

### Step 8. 5⁵-(4-(azetidin-1-yl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

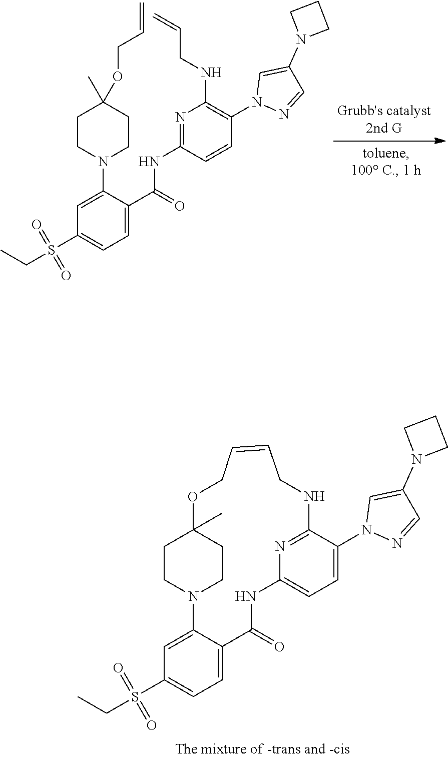 [SMILES: C=CCNc1nc(NC(=O)c2ccc(S(=O)(=O)CC)cc2N2CCC(C)(OCC=C)CC2)ccc1-n1cc(N2CCC2)cn1 ; CCS(=O)(=O)c1ccc2c(c1)N1CCC(C)(CC1)OC/C=C\CNc1nc(ccc1-n1cc(N3CCC3)cn1)NC2=O]

Grubb's catalyst 2nd Generation (34.2 mg, 0.04 mmol) was N-(6-(allylamino)-5-(4-(azetidin-1-yl)-1H-pyrazol-1-yl) pyridin-2-yl)-2-(4-(allyloxy)-4-methylpiperidin-1-yl)-4-(ethylsulfonyl) benzamide (250 mg, 0.40 mmol) in toluene (10 mL) under nitrogen atmosphere. The mixture was stirred at 100° C. for 1 hour. The resulting mixture was concentrated under vacuum. The crude product was purified by preparative TLC, eluted with EA:PE (5:1) to afford the product 5⁵-(4-(azetidin-1-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (20 mg crude). LCMS (ESI-MS) m/z=592.3 [M+H]⁺.

Step 9: Compounds 356 and 357: 5⁵-(4-(azetidin-1-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (20 mg crude) was purified by reversed-phase flash chromatography with the following conditions: column, C18 silica gel; mobile phase, aq. 0.04% TFA in ACN, 10% to 76% gradient in 10 min; detector, UV 254 nm to afford two products:

First eluting isomer (Compound 356): (E)-5⁵-(4-(azetidin-1-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (1.7 mg, 0.61% yield).

Second eluting isomer (Compound 357): (Z)-5⁵-(4-(azetidin-1-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (3.0 mg, 1.0% yield).

### Compound 358.

### Step 1: 1-(1-(tetrahydro-2H-pyran-2-yl)-1H-pyrazol-4-yl)cyclobutan-1-ol

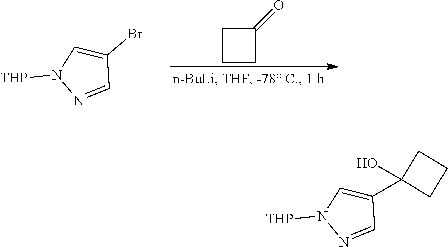

To a stirred mixture of 4-bromo-1-(tetrahydro-2H-pyran-2-yl)-1H-pyrazole (5 g, 20 mmol) in THF (200 mL) was added n-BuLi (2 g, 30 mmol) dropwise at −78° C. under nitrogen atmosphere. The resulting mixture was stirred at −78° C. for additional 0.5 hours. To the above mixture was added cyclobutanone (2 g, 30 mmol). The resulting mixture was stirred at −78° C. for additional 1 hour. The reaction was quenched with sat. NH₄Cl (aq.) at 0° C. The reaction mixture was filtered. The precipitate was filtered off and the filtrate was concentrated. The residue was purified by silica gel column chromatography, eluted with DCM/MeOH (10:1) to afford 1-(1-(tetrahydro-2H-pyran-2-yl)-1H-pyrazol-4-yl)cyclobutan-1-ol (6 g, 90% yield). LCMS (ESI-MS) m/z=223.1 [M+H]⁺.

### Step 2: 4-(1-((tert-butyldimethylsilyl)oxy)cyclobutyl)-1-(tetrahydro-2H-pyran-2-yl)-1H-pyrazole

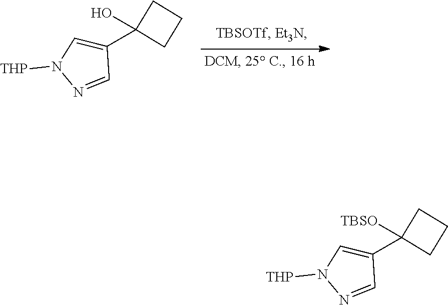

To a stirred solution of 1-(1-(tetrahydro-2H-pyran-2-yl)-1H-pyrazol-4-yl)cyclobutan-1-ol (3 g, 10 mmol) and Et₃N (6.0 mL) in DCM (30 mL) was added TBSOTf (5 g, 20 mmol) in portions at 0° C. under nitrogen atmosphere. The resulting mixture was stirred at 25° C. for additional 16 hours. The reaction was quenched with water at 0° C. The resulting mixture was extracted with DCM (3×50 mL). The resulting mixture was concentrated under vacuum. The crude product was used in the next step directly without further purification 4-(1-((tert-butyldimethylsilyl)oxy)cyclobutyl)-1-(tetrahydro-2H-pyran-2-yl)-1H-pyrazole (5.5 g, crude). LCMS (ESI-MS) m/z=337.2 [M+H]⁺.

### Step 3: 4-(1-((tert-butyldimethylsilyl)oxy)cyclobutyl)-1H-pyrazole

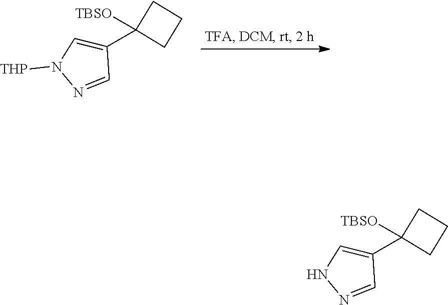

Into a 250 mL round-bottom flask were added 4-(1-((tert-butyldimethylsilyl)oxy)cyclobutyl)-1-(tetrahydro-2H-pyran-2-yl)-1H-pyrazole (5 g, 10 mmol) and TFA (10.0 mL) in DCM (50.0 mL) at room temperature. The mixture was basified to pH 8 with saturated Na₂CO₃ (aq.). The aqueous layer was extracted with DCM (3×100 mL). After filtration, the filtrate was concentrated under reduced pressure. The residue was purified by silica gel column chromatography, eluted with PE/EA (5:1) to afford 4-(1-((tert-butyldimethylsilyl)oxy)cyclobutyl)-1H-pyrazole (400 mg, 9% yield). LCMS (ESI-MS) m/z=253.2 [M+H]⁺.

### Step 4: (Z)-5⁵-(4-(1-((tert-butyldimethylsilyl)oxy)cyclobutyl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

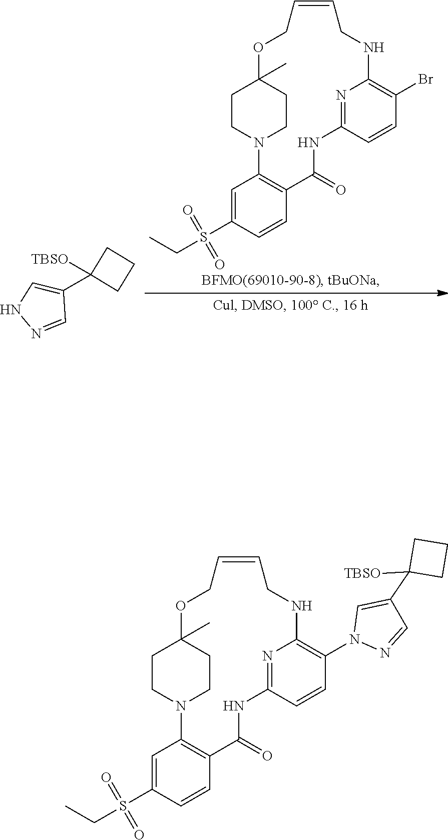

A mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.09 mmol), 4-(1-((tert-butyldimethylsilyl)oxy)cyclobutyl)-1H-pyrazole (69 mg, 0.27 mmol), t-BuONa (35 mg, 36 mmol), N¹,N²-bis(furan-2-ylmethyl)oxalamide (3.0 mg, 0.01 mmol) and CuI (2.0 mg, 0.01 mmol) in DMSO (23 mL) was stirred at 100° C. for 16 hours under a nitrogen atmosphere. The reaction solution was purified by reversed-phase column (48 g) and eluted with ACN in water (NH₄HCO₃ 0.1%) from 0% to 65%. Fractions with desired mass signal were combined and concentrated under vacuum to afford (Z)-5⁵-(4-(1-((tert-butyldimethylsilyl)oxy)cyclobutyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (40 mg, 49% yield). LCMS (ESI-MS) m/z=721.3 [M+H]⁺.

Step 5: Compound 358: 1M TBAF in THF (0.35 mL, 0.35 mmol) was added (Z)-5⁵-(4-(1-((tert-butyldimethylsilyl)oxy)cyclobutyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.07 mmol) in THF (1 mL) at 25° C. The resulting mixture was stirred for 1 hour at room temperature. The resulting mixture was extracted with EA (3×5 mL). The combined organic layers were washed with aq.NH₄Cl (3×5 mL), dried over anhydrous Na₂SO₄. After filtration, the filtrate was concentrated under reduced pressure. The residue was purified by silica gel column chromatography, eluted with PE/EA (1:1) to afford the crude product. The sample (30 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 35% B to 65% B in 8 min; Wave Length: 254 nm/220 nm nm; RT1 (min): 7.31) to afford (Z)-2⁵-(ethylsulfonyl)-5⁵-(4-(1-hydroxycyclobutyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (10.5 mg, 25% yield).

### Compound 359

### Step 1: 2-(1-(tetrahydro-2H-pyran-2-yl)-1H-pyrazol-3-yl) isothiazolidine 1,1-dioxide

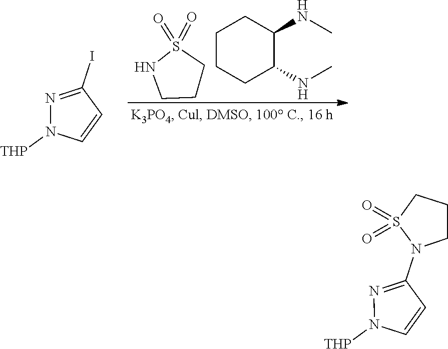

K₃PO₄ (2 g, 10 mmol) was added to the mixture of 3-iodo-1-(tetrahydro-2H-pyran-2-yl)-1H-pyrazole (1 g, 4 mmol), isothiazolidine 1,1-dioxide (1 g, 10 mmol), CuI (70 mg, 0.4 mmol) and (1R,2R)—N¹, N²-dimethylcyclohexane-1,2-diamine (50 mg, 0.4 mmol) in DMSO (10 mL) under nitrogen atmosphere. The mixture was stirred at 100° C. overnight. After cooling to room temperature, the reaction mixture was diluted with H₂O (100 mL) and extracted with EA (3×100 mL). The combined organic layers were washed with brine (300 mL), dried over anhydrous Na₂SO₄, filtered and concentrated under vacuum to afford the crude product. The crude product purified by silica gel column chromatography, eluted with MeOH:DCM (1:10) to afford 2-(1-(tetrahydro-2H-pyran-2-yl)-1H-pyrazol-3-yl) isothiazolidine 1,1-dioxide (1.3 g crude).

### Step 2: 2-(1H-pyrazol-3-yl) isothiazolidine 1,1-dioxide

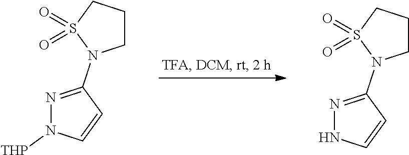

TFA (2 mL) was added to 2-(1-(tetrahydro-2H-pyran-2-yl)-1H-pyrazol-3-yl) isothiazolidine 1,1-dioxide (1.3 g, 4.8 mmol) in DCM (5 mL). The mixture was stirred at room temperature for 2 hours. The resulting mixture was concentrated under vacuum. The residue was dissolved in aq. Na₂CO₃ (20 mL). The resulting mixture was extracted with EA (3×20 mL). The combined organic layers were washed with brine (100 mL), dried over anhydrous Na₂SO₄. After filtration, the filtrate was concentrated under reduced pressure to afford 2-(1H-pyrazol-3-yl) isothiazolidine 1,1-dioxide (483 mg, 54% yield).

### Step 3: Compound 359

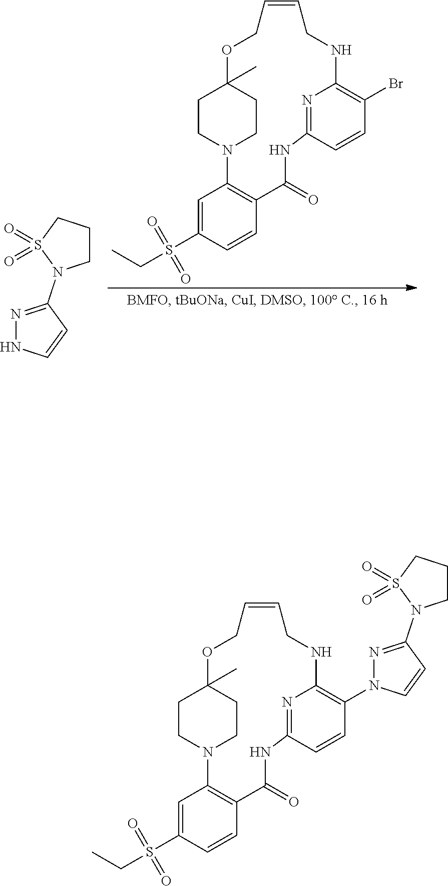

t-BuONa (42 mg, 0.44 mmol) was added to the mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (80 mg, 0.15 mmol), 2-(1H-pyrazol-3-yl) isothiazolidine 1,1-dioxide (160 mg, 0.85 mmol), N¹,N²-bis(furan-2-ylmethyl)oxalamide (4 mg, 0.02 mmol) and CuI (3 mg, 0.02 mmol) in DMSO (20 mL) under nitrogen atmosphere. The mixture was stirred at 100° C. overnight. After cooling to room temperature, the reaction mixture was diluted with H₂O (100 mL) and extracted with EA (3×100 mL). The combined organic layers were washed with brine (300 mL), dried over anhydrous Na₂SO₄, filtered and concentrated under vacuum to afford the crude product. The crude product purified by reversed-phase flash chromatography with the following conditions: Column X-Bridge Prep OBD C18 Column, 30*150 mm, 5 μm; Mobile Phase A: Water(10 mmol/LNH₄HCO₃), Mobile Phase B: ACN; Flow rate: 60 mL/min; Gradient (B %): 12% B to 42% B in 7 min; Wave Length: 254 nm/220 nm nm; RT1 (min): 7; Number Of Runs: 1 to afford (Z)-5⁵-(3-(1,1-dioxidoisothiazolidin-2-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenac-ycloundecaphan-8-en-3-one (9.3 mg, 9.7% yield).

Compound 360 was prepared using the procedure outlined in Compound 359 using (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one to afford (E)-5⁵-(3-(1,1-dioxidoisothiazolidin-2-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenac-ycloundecaphan-8-en-3-one (12.7 mg, 21% yield).

### Compound 361.

K₃PO₄ (60 mg, 0.27 mmol) was added to the mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (50 mg, 0.094 mmol), 2-(1H-pyrazol-3-yl) isothiazolidine 1,1-dioxide (100 mg, 0.53 mmol), CuI (2 mg, 0.01 mmol) and (1R,2R)—N¹,N²-dimethylcyclohexane-1,2-diamine (2 mg, 0.01 mmol) in DMSO (5 mL) under nitrogen atmosphere. The mixture was stirred at 100° C. overnight. After cooling to room temperature, the reaction mixture was diluted with H₂O (50 mL) and extracted with EA (3×50 mL). The combined organic layers were washed with brine (200 mL), dried over anhydrous Na₂SO₄, filtered and concentrated under vacuum to afford the crude product. The crude product purified by reversed-phase flash chromatography, with the following conditions: Column: X-Bridge Prep Shield RP18 OBD C18 Column, 30*150 mm, 51m; Mobile Phase A: Water(10 mmol/LNH₄HCO₃), Mobile Phase B: ACN; Flow rate: 60 mL/min; Gradient (B %): 47% B to77% B in 10 min; Wave Length: 254 nm/220 nm; RT1 (min): 7.6 to afford (E)-5⁵-(3-(1,1-dioxidoisothiazolidin-2-yl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (34.7 mg, 57% yield).

### Compound 362

K₃PO₄ (58 mg, 0.27 mmol) was added to the mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.091 mmol), 4-(1H-pyrazol-4-yl)morpholin-3-one (42 mg, 0.27 mmol), CuI (1.7 mg, 0.0091 mmol) and (1R,2R)—N¹,N²-dimethylcyclohexane-1,2-diamine (1.3 mg, 0.0091 mmol) in DMSO (5 mL) under nitrogen atmosphere. The mixture was stirred at 100° C. overnight. After cooling to room temperature, the reaction mixture was diluted with H₂O (50 mL) and extracted with EA (3×50 mL). The combined organic layers were washed with brine (200 mL), dried over anhydrous Na₂SO₄, filtered and concentrated under vacuum to afford the crude product. The crude product purified by reversed-phase flash chromatography, with the following conditions: Column: X-select CSH Prep Fluoro-Phenyl Column, 30*150 mm, 5 μm; Mobile Phase A: Water(0.1% FA), Mobile Phase B: ACN; Flow rate: 60 mL/min; Gradient (B %): 28% B to 58% B in 7 min; Wave Length: 254 nm/220 nm nm; RT1 (min): 6.6 to afford (Z)-2⁵-(ethylsulfonyl)-1⁴-methyl-5⁵-(3-morpholino-1H-pyrazol-1-yl)-11-oxa-4,6-diaza-5(2,6)-pyridina-1(1,4)-piperidina-2(1,2)-benzenacycloundecaphan-8-en-3-one (9.5 mg, 17% yield).

Compound 363 was prepared according to the procedure outlined in Compound 362 using (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one to afford (E)-2⁵-(ethylsulfonyl)-1⁴-methyl-5⁵-(3-morpholino-1H-pyrazol-1-yl)-11-oxa-4,6-diaza-5(2,6)-pyridina-1(1,4)-piperidina-2(1,2)-benzenacyclounde-caphan-8-en-3-one (8.3 mg, 15% yield).

Compound 364 was prepared according to the procedure outlined in Compound 362 using (E)-5⁵-bromo-2⁵-(ethylsulfonyl)-4,6-diaza-5(2,6)-pyridina-1(1,4)-piperidina-2(1,2)-benzenacycloundecaphan-9-en-3-one to afford (E)-2⁵-(ethylsulfonyl)-5⁵-(3-morpholino-1H-pyrazol-1-yl)-4,6-diaza-5(2,6)-pyridina-1(1,4)-piperidina-2(1,2)-benzenacycloundecaphan-9-en-3-one (8.0 mg, 14% yield).

### Compound 365.

### Step 1: (E)-N-(5⁵-(3-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide

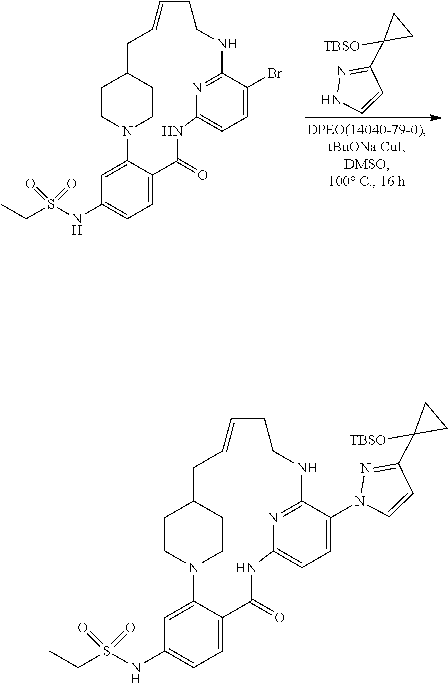 [SMILES: CCS(=O)(=O)Nc1ccc2c(c1)N1CCC(C/C=C/CCNc3nc(ccc3Br)NC2=O)CC1 ; CC(C)(C)[Si](C)(C)OC1(c2cc[nH]n2)CC1 ; CCS(=O)(=O)Nc1ccc2c(c1)N1CCC(C/C=C/CCNc3nc(ccc3-n3ccc(C4(O[Si](C)(C)C(C)(C)C)CC4)n3)NC2=O)CC1]

To a solution of (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (50 mg, 0.09 mmol) in DMSO (1.5 mL) added 3-(1-((tert-butyldimethylsilyl)oxy)cyclopropyl)-1H-pyrazole (65 mg, 0.27 mmol) and tBuONa (35 mg, 0.36 mmol) under nitrogen atmosphere, then added N,N-Bis(2-phenylethyl)oxamide (1 mg, 0.01 mmol) and CuI (1 mg, 0.01 mmol). After stirring for 16 hours at 100° C. under nitrogen atmosphere. The crude product was purified by reversed-phase column (25 g) and eluted with ACN in water (NH₄HCO₃ 0.1%) from 0% to 85%. Fractions with desired mass signal were combined and concentrated under vacuum to afford the desired product (E)-N-(5⁵-(3-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (15 mg, 19% yield). LCMS (ESI-MS) m/z=239.1 [M+H]⁺.

Step 2: Compound 365:1M TBAF in THF (0.04 mL, 0.04 mmol) was added (E)-N-(5⁵-(3-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (15 mg, 0.02 mmol) in THF (4 mL) at 25° C. for 1 hour. The resulting mixture was extracted with EA (3×5 mL). The combined organic layers were washed with aq.NH₄Cl (3×5 mL), dried over anhydrous Na₂SO₄. After filtration, the filtrate was concentrated under reduced pressure. The residue was purified by silica gel column chromatography, eluted with PE/EA (1:2) to afford Product (20 mg, purity 80%). The crude product (20 mg) was purified by Prep-HPLC with the following conditions (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 51% B to 81% B in 10 min; Wave Length: 254 nm/220 nm nm; RT1 (min): 7.18) to afford (E)-N-(5⁵-(3-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 17% yield).

### Compound 366.

### Step 1: (E)-5⁵-(4-(1-((tert-butyldimethylsilyl)oxy)cyclobutyl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

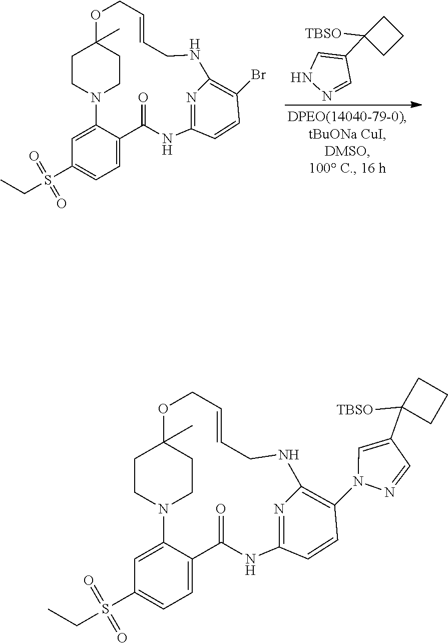 [SMILES: CCS(=O)(=O)c1ccc2c(c1)N1CCC(C)(CC1)OC/C=C/CNc1nc(ccc1Br)NC2=O ; CC(C)(C)[Si](C)(C)OC1(c2cn[nH]c2)CCC1 ; CCS(=O)(=O)c1ccc2c(c1)N1CCC(C)(CC1)OC/C=C/CNc1nc(ccc1-n1cc(C3(O[Si](C)(C)C(C)(C)C)CCC3)cn1)NC2=O]

Into a 8 mL vial were added (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.09 mmol), 4-(1-((tert-butyldimethylsilyl)oxy)cyclobutyl)-1H-pyrazole (69 mg, 0.27 mmol), sodium 2-methylpropan-2-olate (35 mg, 0.36 mmol), N,N-Bis(2-phenylethyl)oxamide (3.0 mg, 0.01 mmol) and CuI (2.0 mg, 0.01 mmol) in DMSO (1.5 mL) at room temperature under nitrogen atmosphere. The resulting mixture was stirred for 16 hours at 100° C. under nitrogen atmosphere. The resulting mixture was extracted with EA (3×5 mL). The combined organic layers were washed with water (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 10% to 90% gradient in 10 min to afford (E)-5⁵-(4-(1-((tert-butyldimethylsilyl)oxy)cyclobutyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (10 mg, 14% yield).

Step 2: Compound 366: 1M TBAF in THF (11 mg, 0.04 mmol) was added (E)-5⁵-(4-(1-((tert-butyldimethylsilyl)oxy)cyclobutyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (15 mg, 0.02 mmol) in THF (4 mL) at 25° C. The resulting mixture was stirred for 1 hour at 25° C. The resulting mixture was extracted with EA (3×5 mL). The combined organic layers were washed with aq.NH₄Cl (3×5 mL), dried over anhydrous Na₂SO₄. After filtration, the filtrate was concentrated under reduced pressure. The residue was purified by silica gel column chromatography, eluted with PE/EA (1:2) to afford Product (20 mg, purity 80%). The crude product (20 mg) was purified by Prep-HPLC with the following conditions (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 51% B to 81% B in 10 min; Wave Length: 254 nm/220 nm nm; RT1 (min): 7.18) to afford (E)-2⁵-(ethylsulfonyl)-5⁵-(4-(1-hydroxycyclobutyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (1.4 mg, 9.5% yield).

Compound 367 was prepared using the procedure outlined in Compound 366 using (E)-5⁵-bromo-2⁵-(ethylsulfonyl)-4,6-diaza-5(2,6)-pyridina-1(1,4)-piperidina-2(1,2)-benzenacycloundecaphan-9-en-3-one to afford (E)-2⁵-(ethylsulfonyl)-55-(4-(1-hydroxycyclobutyl)-1H-pyrazol-1-yl)-4,6-diaza-5(2,6)-pyridina-1(1,4)-piperidina-2(1,2)-benzenacycloundecaphan-9-en-3-one (15 mg, 18% yield).

### Compound 368.

### Step 1: 3-(cyclopropylsulfonyl)-1-(tetrahydro-2H-pyran-2-yl)-1H-pyrazole

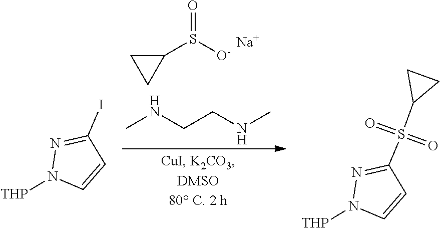

CuI (0.07 g, 0.4 mmol) and N¹,N²-dimethylethane-1,2-diamine (0.03 g, 0.4 mmol) were added to a mixture of 3-iodo-1-(tetrahydro-2H-pyran-2-yl)-1H-pyrazole (1 g, 4 mmol), sodium cyclopropanesulfinate (0.6 g, 4 mmol) and K₂CO₃ (1 g, 10 mmol) in DMSO (10 mL) at room temperature under nitrogen atmosphere. The resulting mixture was stirred for 2 hours at 80° C. under nitrogen atmosphere. The residue was purified by reversed-phase flash chromatography with the following conditions: column, C18 silica gel; mobile phase, H₂O in ACN, 10% to 25% gradient in 10 min; detector, UV 254 nm to afford 3-(cyclopropylsulfonyl)-1-(tetrahydro-2H-pyran-2-yl)-1H-pyrazole (800 mg, 70% yield). LCMS (ESI-MS) m/z=257.0 [M+H]⁺.

### Step 2: 3-(cyclopropylsulfonyl)-1H-pyrazole

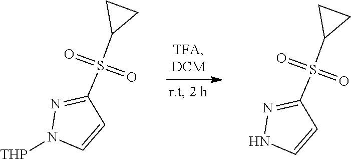

TFA (4 mL) was added into a mixture of 3-(cyclopropylsulfonyl)-1-(tetrahydro-2H-pyran-2-yl)-1H-pyrazole (800 mg, 3.12 mmol) in DCM (8 mL) at room temperature. The resulting mixture was stirred for 2 hours at room temperature. The mixture was neutralized to pH 9 with sodium carbonate aqueous solution. The reaction was quenched with water (50 mL). The aqueous layer was extracted with EA (3×50 mL), and wash the organic phase with saturated saline solution (150 mL). The resulting mixture was dried with Na₂SO₄. The resulting mixture was concentrated under reduced pressure to afford the crude product 3-(cyclopropylsulfonyl)-1H-pyrazole (700 mg, 100% yield crude). LCMS (ESI-MS) m/z=173.0 [M+H]⁺.

Step 3: Compound 368: CuI (1.7 mg, 0.01 mmol) and DPEO (2.7 mg, 0.01 mmol) were added into a mixture of 3-(cyclopropylsulfonyl)-1H-pyrazole (16 mg, 0.09 mmol), (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.09 mmol) and tBuONa (26 mg, 0.27 mmol) in DMSO (0.5 mL) at room temperature under nitrogen atmosphere. The resulting mixture was stirred for overnight at 100° C. under nitrogen atmosphere. The reaction was quenched with water (5 mL). The aqueous layer was extracted with EA (3×5 mL) and the combined organic phase was washed with saturated saline solution (15 mL). The resulting mixture was dried with Na₂SO₄. The resulting mixture was concentrated under reduced pressure and the resulting residue was purified by preparative TLC ([PE:EA][1:3]) to afford the crude product. This material was further purified by preparative HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 36% B to 60% B in 8 min; Wave Length: 254 nm/220 nm nm; RT1 (min): 6.2 to afford (Z)-5⁵-(3-(cyclopropylsulfonyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (16.5 mg, 8.5% yield).

### Compound 369.

### Step 1: 3-(pyrrolidin-1-yl)-1-(tetrahydro-2H-pyran-2-yl)-1H-pyrazole

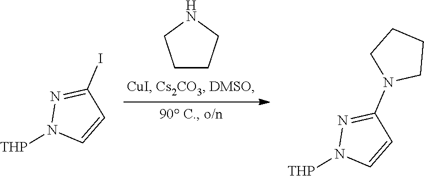

Cs₂CO₃ (4 g, 10 mmol) was added to the mixture of (1R,2R)—N¹, N²-dimethylcyclohexane-1,2-diamine (0.6 g, 4 mmol), pyrrolidine (1 g, 20 mmol), CuI (1 g, 5 mmol) and 3-iodo-1-(tetrahydro-2H-pyran-2-yl)-1H-pyrazole (1 g, 4 mmol) in DMSO (10 mL) under nitrogen atmosphere. The mixture was stirred at 90° C. overnight. The resulting mixture was filtered, the filter cake was washed with DCM (3×20 mL). The filtrate was concentrated under reduced pressure. The residue was purified by reversed-phase flash chromatography with the following conditions: Column, C18 silica gel; mobile phase, aq. 0.04% TFA in ACN, 10% to 68% gradient in 10 min; detector, UV 254 nm to afford the product 3-(pyrrolidin-1-yl)-1-(tetrahydro-2H-pyran-2-yl)-1H-pyrazole (190 mg, 20% yield). LCMS (ESI-MS) m/z=222.2 [M+H]⁺.

### Step 2: 4-(1-((tert-butyldimethylsilyl) oxy) vinyl)-1-((2-(trimethylsilyl) ethoxy) methyl)-1H-pyrazole

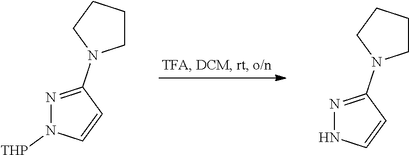

TFA (0.3 mL) was added to the mixture of 3-(pyrrolidin-1-yl)-1-(tetrahydro-2H-pyran-2-yl)-1H-pyrazole (190 mg, 0.86 mmol) in DCM (5 mL). The mixture was stirred at room temperature for 16 hours. The resulting mixture was concentrated under vacuum to afford the crude product. The residue was dissolved in aq. NH₄HCO₃ (5 mL). The resulting mixture was extracted with EA (3×5 mL). The combined organic layers were washed with brine (15 mL), dried over anhydrous Na₂SO₄. After filtration, the filtrate was concentrated under reduced pressure to afford the product 3-(pyrrolidin-1-yl)-1H-pyrazole (150 mg, crude). LCMS (ESI-MS) m/z=138.1 [M+H]⁺.

### Step 3: Compound 369:

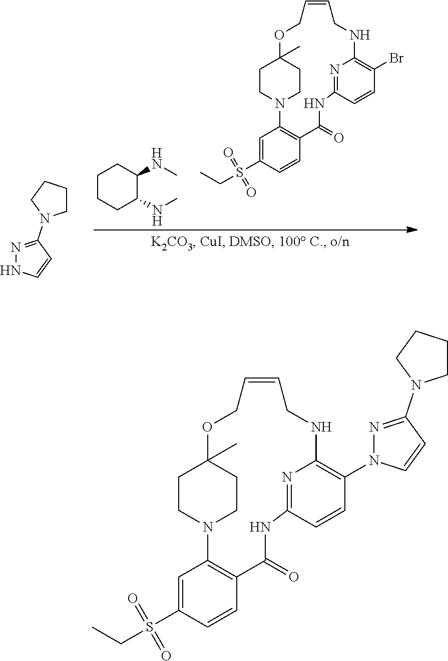

K₂CO₃ (7.5 mg, 0.055 mmol) was added to the mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (30 mg, 0.055 mmol), 3-(pyrrolidin-1-yl)-1H-pyrazole (22 mg, 0.16 mmol), (1R,2R)—N¹,N²-dimethylcyclohexane-1,2-diamine (3 mg, 0.2 mmol) in 1,4-dioxane (3 mL) under a nitrogen atmosphere. The mixture was stirred at 100° C. for 16 hours. The reaction mixture was purified by Prep-HPLC with following condition (Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 54% B to 84% B in 8 min; Wave Length: 254 nm/220 nm n; RT1 (min): 7.92) to afford (Z)-2⁵-(ethylsulfonyl)-1⁴-methyl-5⁵-(3-(pyrrolidin-1-yl)-1H-pyrazol-1-yl)-11-oxa-4,6-diaza-5(2,6)-pyridina-1(1,4)-piperidina-2(1,2)-benzenacycloundecaphan-8-en-3-one (2.2 mg, 6.6% yield).

Compound 370 was prepared according to the procedure described in Compound 369 using (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one to afford the product (E)-2⁵-(ethylsulfonyl)-1⁴-methyl-5⁵-(3-(pyrrolidin-1-yl)-1H-pyrazol-1-yl)-11-oxa-4,6-diaza-5(2,6)-pyridina-1(1,4)-piperidina-2(1,2)-benzenacyc-loundecaphan-8-en-3-one (5.9 mg, 17% yield).

Compound 371 was prepared according to General Procedure 2 using Compound 300 and 3-(pyrrolidin-1-yl)-1H-pyrazole to afford (E)-2⁵-(ethylsulfonyl)-55-(3-(pyrrolidin-1-yl)-1H-pyrazol-1-yl)-4,6-diaza-5(2,6)-pyridina-1(1,4)-piperidina-2(1,2)-benzenacycloundecaphan-9-en-3-one to (1.8 mg, 5.4% yield).

### Compound 372.

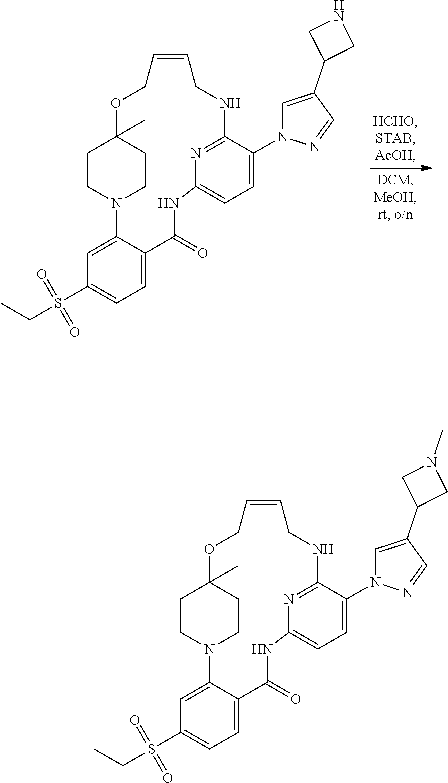 [SMILES: CCS(=O)(=O)c1ccc2c(c1)N1CCC(C)(CC1)OC/C=C\CNc1nc(ccc1-n1cc(C3CNC3)cn1)NC2=O ; CCS(=O)(=O)c1ccc2c(c1)N1CCC(C)(CC1)OC/C=C\CNc1nc(ccc1-n1cc(C3CN(C)C3)cn1)NC2=O]

STAB (3 mg, 0.02 mmol) was added to the mixture of (Z)-5⁵-(4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaph-an-8-en-3-one (6 mg, 0.01 mmol), formaldehyde (2 mg, 37% Wₜ, 0.03 mmol) and AcOH (1 mg, 0.02 mmol) in DCM (1 mL). The mixture was stirred at room temperature for 1 hour and then was poured into water (2 mL). The resulting mixture was extracted with EA (3×2 mL), dried over anhydrous Na₂SO₄, and concentrated under reduced pressure. The resulting residue was purified by Prep-HPLC with following condition (Column: X-Bridge Prep OBD C18 Column, 30*150 mm, 5 μm; Mobile Phase A: Water(10 mmol/LNH₄HCO₃), Mobile Phase B: ACN; Flow rate: 60 mL/min mL/min; Gradient (B %): 25% B to 55% B in 11 min; Wave Length: 254 nm/220 nm n; RT1 (min): 9.5; 10.5) to afford the product (Z)-2⁵-(ethylsulfonyl)-1⁴-methyl-5⁵-(4-(1-methylazetidin-3-yl)-1H-pyrazol-1-yl)-11-oxa-4,6-diaza-5(2,6)-pyridina-1(1,4)-piperidina-2(1,2)-benzenacycloundecaphan-8-en-3-one (1.7 mg, 30% yield).

### Compound 373.

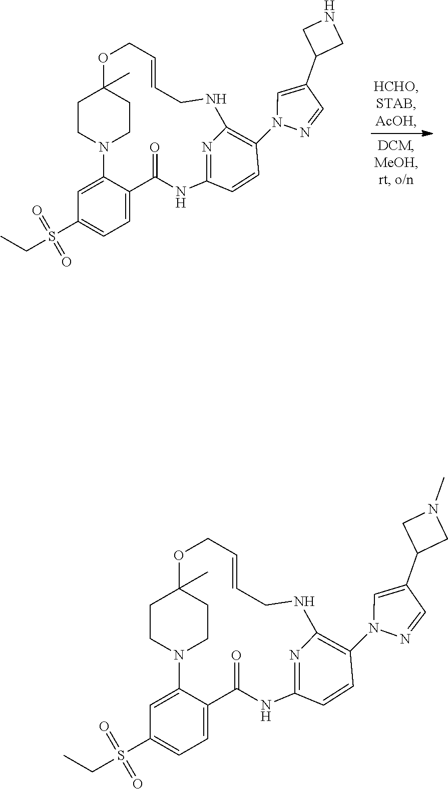 [SMILES: CCS(=O)(=O)c1ccc2c(c1)N1CCC(C)(CC1)OC/C=C/CNc1nc(ccc1-n1cc(C3CNC3)cn1)NC2=O ; CCS(=O)(=O)c1ccc2c(c1)N1CCC(C)(CC1)OC/C=C/CNc1nc(ccc1-n1cc(C3CN(C)C3)cn1)NC2=O]

STAB (2 mg, 0.01 mmol) was added to the mixture of (F)-5⁵ (4-(azetidin-3-yl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecap-han-8-en-3-one (4 mg, 0.005 mmol), AcOH (2 mg, 0.03 mmol) and formaldehyde (2 mg, 37% Wt, 0.02 mmol) in DCM (1 mL) under nitrogen atmosphere. The mixture was stirred at room temperature for 1 hour. The mixture was purified by Prep-HPLC with following condition (Column: X-Bridge Prep OBD C18 Column, 30*150 mm, 5 μm; Mobile Phase A: Water(10 mmol/LNH₄HCO₃), Mobile Phase B: ACN; Flow rate: 60 mL/min mL/min; Gradient (B %): 25% B to 55% B in 11 min; Wave Length: 254 nm/220 nm nm; RT1 (min): 9.5; 10.5) to afford the product (E)-2⁵-(ethylsulfonyl)-1⁴-methyl-5⁵-(4-(1-methylazetidin-3-yl)-1H-pyrazol-1-yl)-11-oxa-4,6-diaza-5(2,6)-pyridina-1(1,4)-piperidina-2(1,2)-benzenacycloundecaphan-8-en-3-one (1.9 mg, 50% yield).

### Compound 374.

### Step 1: (Z)-55-(3-(1-((tert-butyldimethylsilyl)oxy)cyclopropyl)-1H-pyrazol-1-yl)-2⁵-(ethylsulfonyl)-14-methyl-11-oxa-4,6-diaza-5(2,6)-pyridina-1(1,4)-piperidina-2(1,2)-benzenacycloundecaphan-8-en-3-one

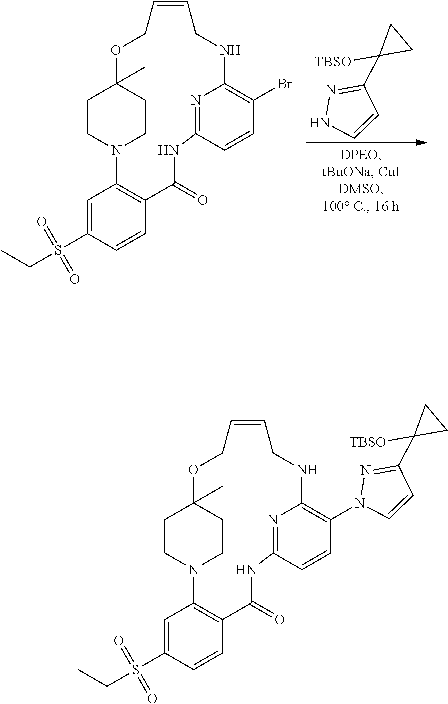 [SMILES: CCS(=O)(=O)c1ccc2c(c1)N1CCC(C)(CC1)OC/C=C\CNc1nc(ccc1Br)NC2=O ; CC(C)(C)[Si](C)(C)OC1(c2cc[nH]n2)CC1 ; CCS(=O)(=O)c1ccc2c(c1)N1CCC(C)(CC1)OC/C=C\CNc1nc(ccc1-n1ccc(C3(O[Si](C)(C)C(C)(C)C)CC3)n1)NC2=O]

A solution of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (40 mg, 0.07 mmol) in DMSO (1.5 mL) was added to 3-(1-((tert-butyldimethylsilyl)oxy)cyclopropyl)-1H-pyrazole (52 mg, 0.22 mmol) and tBuONa (28 mg, 0.29 mmol) under a nitrogen atmosphere DPEO (3 mg, 0.01 mmol) and CuI (2 mg, 0.01 mmol) were added and the mixture was stirred for 16 hours at 100° C. under a nitrogen atmosphere. The residue was purified by reversed-phase flash chromatography with the following conditions: column, C18 silica gel; mobile phase, ACN in Water (0.1% NH₃·H₂O), 20% to 80% gradient in 30 min; detector, UV 254 nm. to afford (Z)-5⁵-(3-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (15 mg, 23% yield). LCMS (ESI-MS) m/z=708.1 [M+H]⁺.

Step 2: Compound 374: 1 M TBAF in THF (0.03 mL, 0.03 mmol) was added (Z)-5⁵-(3-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (10 mg, 0.01 mmol) in THF (4 mL) at 25° C. The resulting mixture was stirred for 1 hour at 25° C. The resulting mixture was extracted with EA (3×5 mL). The combined organic layers were washed with water (3×5 mL), dried over anhydrous Na₂SO₄. After filtration, the filtrate was concentrated under reduced pressure and the resulting residue was purified by silica gel column chromatography, eluted with PE/EA (1:2) to afford the crude product. This material was further purified via Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 35% B to 65% B in 8 min; Wave Length: 254 nm/220 nm n; RT1 (min): 7.4) to afford (Z)-2⁵-(ethylsulfonyl)-5⁵-(3-(1-hydroxycyclopropyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (1.2 mg, 13% yield).

### Compound 375.

### Step 1: (E)-2⁵-(ethylsulfonyl)-5⁵-(3-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one

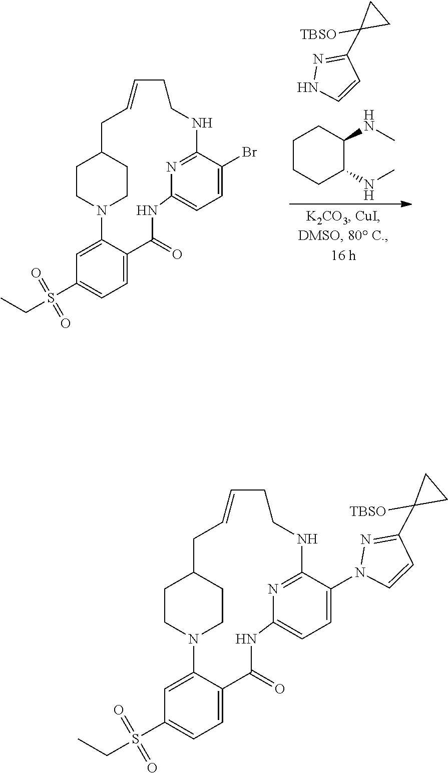

(1R,2R)—N¹,N²-dimethylcyclohexane-1,2-diamine (1.0 mg, 0.01 mmol) and CuI (2 mg, 0.01 mmol) were added into a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (40 mg, 0.08 mmol), 3-(1-((tert-butyldimethylsilyl)oxy)cyclopropyl)-1H-pyrazole (54 mg, 0.22 mmol) and K₂CO₃ (31 mg, 0.22 mmol) in DMSO (0.3 mL) at room temperature under nitrogen atmosphere. The resulting mixture was stirred for 16 hours at 80° C. under nitrogen atmosphere. The reaction was purified by reverse phase column and eluted with ACN in water (0.1% NH₄HCO₃) from 0% to 85%. Fractions with desired mass signal were combined and concentrated under vacuum to afford (E)-2⁵-(ethylsulfonyl)-5⁵-(3-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one (10 mg, 17% yield). LCMS (ESI-MS) m/z=691.4 [M+H]⁺.

Step 2: Compound 375:1M TBAF in THF (0.04 mL, 0.04 mmol) was added (E)-5⁵-(3-(1-((tert-butyldimethylsilyl) oxy) cyclopropyl)-1H-pyrazol-1-yl)-2⁵-(ethylsulfonyl)-4,6-diaza-5(2,6)-pyridina-1(1,4)-pieridina-2(1,2)-benzenacycloundecaphan-9-en-3-one (10 mg, 0.02 mmol) in THF (3 mL) at 25° C. The resulting mixture was stirred for 1 hour at room temperature. The resulting mixture was extracted with EA (3×5 mL). The combined organic layers were washed with aq.NH₄Cl (3×5 mL), dried over anhydrous Na₂SO₄. After filtration, the filtrate was concentrated under reduced pressure. The residue was purified by Preparative TLC (PE/EA 1:1) to afford (E)-2⁵-(ethylsulfonyl)-5⁵-(3-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one (2.0 mg, 21% yield).

### Compound 376.

### Step 1: (Z)—N-(5⁵-(3-(1-((tert-butyldimethylsilyl) oxy) cyclopropyl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl) ethanesulfonamide

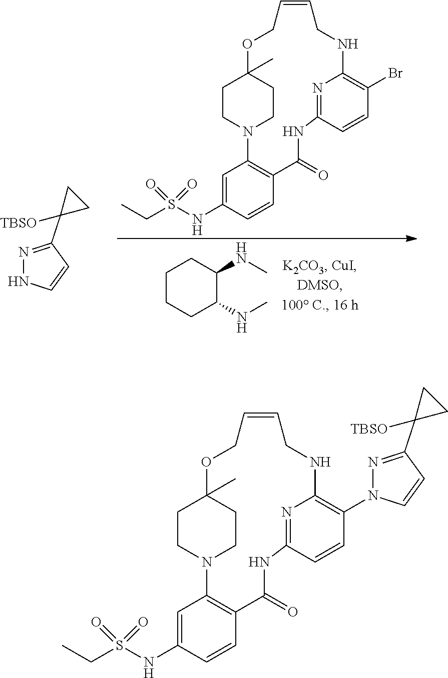

K₃PO₄ (4.0 equiv) was added to the mixture of (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.0 equiv), 3-(1-((tert-butyldimethylsilyl)oxy)cyclopropyl)-1H-pyrazole (1.0 equiv), (1R,2R)—N¹, N²-dimethylcyclohexane-1,2-diamine (0.05 equiv) and CuI (0.05 equiv) in DMSO (10 V) under a nitrogen atmosphere. The mixture was stirred at 100° C. overnight. The reaction mixture was purified by reversed-phase flash chromatography with the following conditions: Column, C18 silica gel; mobile phase, aq. 0.1% NH₄HCO₃ in ACN, 10% to 80% gradient in 10 min; detector, UV 220 nm to afford the product (Z)—N-(5⁵-(3-(1-((tert-butyldimethylsilyl)oxy)cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (28% yield).

Step 2: Compound 376: 1M TBAF in THF (2.0 equiv) was added to ((Z)—N-(5⁵-(3-(1-((tert-butyldimethylsilyl) oxy) cyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (1.0 equiv) in THF (5 mL). The mixture was stirred at room temperature for 1 hour. The reaction was poured into aq. NH₄Cl (10 mL) at temperature. The resulting mixture was extracted with EA (20 V). The combined organic layers were washed with aq. NH₄Cl (3×20 V mL), dried over anhydrous Na₂SO₄. After filtration, the filtrate was concentrated under reduced pressure. The crude product was purified by preparative TLC, eluted with MeOH:DCM (1:20) to afford the product (Z)—N-(5⁵-(3-(1-hydroxycyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (45% yield).

### Compound 377.

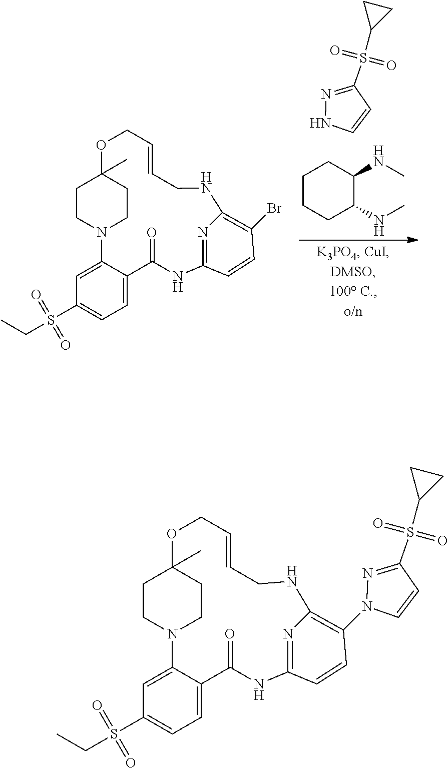

CuI (2 mg, 0.01 mmol) and (1R,2R)—N¹,N²-dimethylcyclohexane-1,2-diamine (2 mg, 0.01 mmol) were added into a mixture of (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.09 mmol), 3-(cyclopropylsulfonyl)-1H-pyrazole (16 mg, 0.09 mmol) and K₃PO₄ (58 mg, 0.27 mmol) in DMSO (0.5 mL) at room temperature under nitrogen atmosphere. The resulting mixture was stirred for overnight at 100° C. under nitrogen atmosphere. The reaction was quenched with water (5 mL). The aqueous layer was extracted with EA (3×5 mL), and wash the organic phase with saturated saline solution (15 mL). The resulting mixture was dried with Na₂SO₄. The resulting mixture was concentrated under reduced pressure. The residue was purified by Preparative TLC ([PE:EA][1:3]) to afford the crude product. The crude was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 36% B to 66% B in 10 min; Wave Length: 254 nm/220 nm nm; RT1 (min): 7.6 to afford (E)-5⁵-(3-(cyclopropylsulfonyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (12.4 mg, 21% yield).

Compound 378 was prepared according to the procedure outlined in Compounds 377 using (E)-5⁵-bromo-2⁵-(ethylsulfonyl)-4,6-diaza-5(2,6)-pyridina-1(1,4)-piperidina-2(1,2)-benzenacycloundecaphan-9-en-3-one to afford (E)-5⁵-(3-(cyclopropylsulfonyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (27.8 mg, 46% yield).

### Compound 379.

### Step 1: 1-((2-(trimethylsilyl) ethoxy) methyl)-1H-pyrazole-4-carbaldehyde

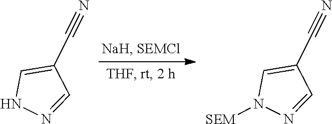 [SMILES: N#Cc1cn[nH]c1 ; C[Si](C)(C)CCOCn1cc(C#N)cn1]

To a solution of 1H-pyrazole-4-carbonitrile (5 g, 50 mmol) in THF (50 mL) was added NaH (2 g, 80 mmol) at 0° C. The mixture was stirred for 30 min, SEMCl (10 g, 60 mmol) was added and the mixture was allowed to warm to room temperature and stirred for 2 hours. The reaction mixture was quenched by NH₄Cl (50 mL) and extracted with EA (3×50 mL). The combined organic layers were washed with brine (200 mL), dried with anhydrous Na₂SO₄, filtered and concentrated under vacuum to afford the crude product. The crude product was dissolved in DCM. Then the solution was uploaded to Flash Column Silica-CS (120 g) and eluting with EA in PE from 0% to 80% with 80 mL/min flow rate. Fractions with desired mass signal were combined and concentrated under vacuum to afford the desired product 1-((2-(trimethylsilyl) ethoxy) methyl)-1H-pyrazole-4-carbonitrile (12 g, 80% yield). LCMS (ESI-MS) m/z=224.10 [M+H]⁺.

### Step 2: 1-(1-((2-(trimethylsilyl) ethoxy) methyl)-1H-pyrazol-4-yl) cyclopropan-1-amine

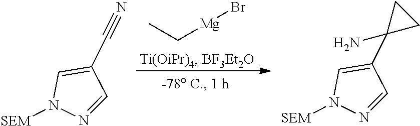

To a solution of 1-((2-(trimethylsilyl) ethoxy) methyl)-1H-pyrazole-4-carbonitrile (4 g, 20 mmol) in THF (40 mL) was added Ti(OiPr)₄ (6 g, 0.02 mol) and ethylmagnesium bromide (120 g, 880 mmol) at −78° C. The mixture was stirred for 30 min. BF₃Et₂O (6 g, 0.04 mol) was added and the mixture was allowed at −78° C. and stirred for 1 hour. The reaction mixture was diluted with NH₄Cl (aq.) (60 mL) and extracted with EA (3×60 mL). The combined organic layers were washed with brine (180 mL), dried over anhydrous Na₂SO₄, filtered and concentrated under vacuum to afford the crude product. The crude product was purified by reversed-phase column (90 g) and eluted with ACN in water (NH₄HCO₃ 0.1%) from 0% to 55%. Fractions with desired mass signal were combined and concentrated under vacuum to afford to 1-(1-((2-(trimethylsilyl) ethoxy) methyl)-1H-pyrazol-4-yl) cyclopropan-1-amine (1 g, 20% yield). LCMS (ESI-MS) m/z=254.15 [M+H]⁺.

### Step 3: tert-butyl (1-(1-((2-(trimethylsilyl) ethoxy) methyl)-1H-pyrazol-4-yl) cyclopropyl) carbamate

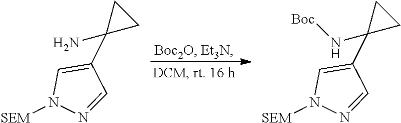 [SMILES: C[Si](C)(C)CCOCn1cc(C2(N)CC2)cn1 ; CC(C)(C)OC(=O)NC1(c2cnn(COCC[Si](C)(C)C)c2)CC1]

To a stirred solution of Boc₂O (215 mg, 0.98 mmol) in DCM (1 mL) was added 1-(1-((2-(trimethylsilyl) ethoxy) methyl)-1H-pyrazol-4-yl) cyclopropan-1-amine (100 mg, 0.39 mmol) and Et₃N (120 mg, 1.18 mmol). The resulting mixture was stirred for 16 hours at room temperature. After completion of reaction, the reaction was quenched with water (5 mL) and the aqueous layer was extracted with DCM (3×5 mL). The combined organic layers were washed with brine (15 mL), dried over anhydrous Na₂SO₄, filtered and concentrated under vacuum to afford the crude product. The residue was purified by silica gel column chromatography, eluted with PE:EA=3:1 to afford tert-butyl (1-(1-((2-(trimethylsilyl) ethoxy) methyl)-1H-pyrazol-4-yl) cyclopropyl) carbamate (110 mg, 63% yield). LCMS (ESI-MS) m/z=354.25 [M+H]⁺.

### Step 4: tert-butyl (1-(1H-pyrazol-4-yl) cyclopropyl) carbamate

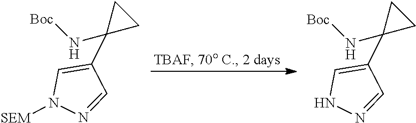 [SMILES: CC(C)(C)OC(=O)NC1(c2cnn(COCC[Si](C)(C)C)c2)CC1 ; CC(C)(C)OC(=O)NC1(c2cn[nH]c2)CC1]

2M TBAF in THF (2 mL) was added to tert-butyl (1-(1-((2-(trimethylsilyl) ethoxy) methyl)-1H-pyrazol-4-yl) cyclopropyl) carbamate (110 mg, 0.3 mmol). The mixture was stirred at 70° C. for 2 days. The reaction mixture was diluted with NH₄Cl (aq.) (10 mL) and extracted with EA (10 mL). The combined organic layers were washed with NH₄Cl (aq.) (3×10 mL), dried over anhydrous Na₂SO₄, filtered and concentrated under vacuum to afford the crude product. The crude product was purified by reversed-phase column (40 g) and eluted with ACN in water (NH₄HCO₃ 0.1%) from 0% to 25%. Fractions with desired mass signal were combined and concentrated under vacuum to afford to tert-butyl (1-(1H-pyrazol-4-yl) cyclopropyl) carbamate (30 mg, 19% yield). LCMS (ESI-MS) m/z=224.3 [M+H]⁺.

### Step 5: tert-butyl (E)-(1-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl) cyclopropyl) carbamate

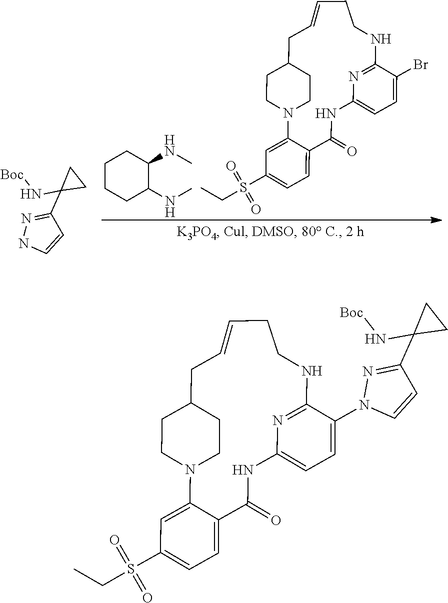

(1R,2R)—N¹,N²-dimethylcyclohexane-1,2-diamine (1 mg, 0.005 mmol) and CuI (1 mg, 0.005 mmol) were added to a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (30 mg, 0.006 mmol), tert-butyl (1-(1H-pyrazol-4-yl) cyclopropyl) carbamate (35 mg, 0.13 mmol) and K₃PO₄ (36 mg, 0.17 mmol) in DMSO (1 mL) at room temperature under nitrogen atmosphere. The resulting mixture was stirred for 2 hours at 80° C. under nitrogen atmosphere. After cooling to room temperature, the reaction mixture was diluted with H₂O (3 mL) and extracted with EA (3×3 mL). The combined organic layers were washed with brine (10 mL), dried over anhydrous Na₂SO₄, filtered and concentrated under vacuum to afford the crude product. The crude product was purified by reversed-phase column (90 g) and eluted with ACN in water (NH₄HCO₃ 0.1%) from 0% to 80%. Fractions with desired mass signal were combined and concentrated under vacuum to afford tert-butyl (E)-(1-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl) cyclopropyl) carbamate (20 mg, 42% yield). LCMS (ESI-MS) m/z=676.3 [M+H]⁺.

Step 6: Compound 379: TFA (0.2 mL) was added to a stirred mixture of tert-butyl (E)-(1-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl) cyclopropyl) carbamate (20 mg, 0.03 mmol) in DCM (1 mL). The resulting mixture was stirred at room temperature for 1 hour and concentrated to afford the crude product, which was purified by Prep-HPLC with following condition (Column: XBridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 43% B to 73% B in 8 min; Wave Length: 254 nm/220 nm nm; RT1 (min): 7.37) to afford (E)-5⁵-(4-(1-aminocyclopropyl)-1H-pyrazol-1-yl)-2⁵-(ethylsulfonyl)-4,6-diaza-5(2,6)-pyridina-1(1,4)-piperidina-2(1,2) benzenacycloundecaphan-9-en-3-one (7.5 mg, 44% yield).

### Compound 380.

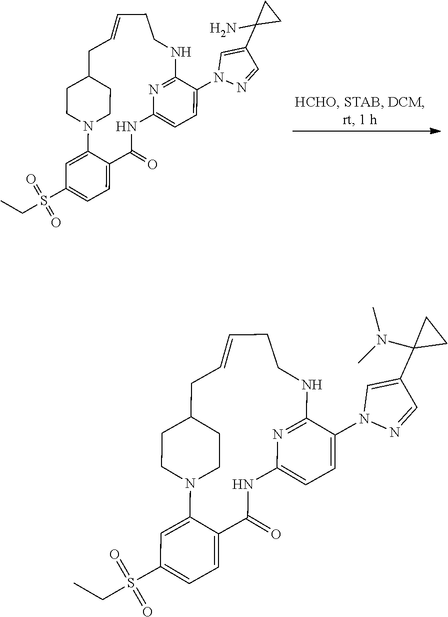 [SMILES: CCS(=O)(=O)c1ccc2c(c1)N1CCC(C/C=C/CCNc3nc(ccc3-n3cc(C4(N)CC4)cn3)NC2=O)CC1 ; CCS(=O)(=O)c1ccc2c(c1)N1CCC(C/C=C/CCNc3nc(ccc3-n3cc(C4(N(C)C)CC4)cn3)NC2=O)CC1]

STAB (37 mg, 0.17 mmol) was added into a mixture of Compound 379, (E)-5⁵-(4-(1-aminocyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one, (50 mg, 0.087 mmol) and HCHO (6.5 mg, 0.22 mmol) in DCM (2 mL) at room temperature. The resulting mixture was stirred at room temperature for 1 hour. Then quenched with solution of water (3 mL), extracted with DCM (3×3 mL). The combined organic layers were washed with brine (9 mL), dried with anhydrous Na₂SO₄, filtered and concentrated under vacuum. The crude product (30 mg) was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 58% B to 78% B in 10 min; Wave Length: 254 nm/220 nm nm; RT1 (min): 8.12) to afford (E)-5⁵-(4-(1-(dimethylamino)cyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (4.7 mg, 8.9% yield).

Compound 381 was synthesized using the synthetic route described in Compound 376 using (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide in Step 1 to afford (E)-N-(5⁵-(3-(1-hydroxycyclopropyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (6.6 mg, 43% yield).

Compound 382 was synthesized using the synthetic route described in Compound 377 using (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide to afford (Z)—N-(5⁵-(3-(cyclopropylsulfonyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (10.4 mg, 18% yield).

Compound 383 was synthesized using the synthetic route described in Compound 377 using (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide to afford (E)-N-(5⁵-(3-(cyclopropylsulfonyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (20.7 mg, 36% yield).

Compound 384 was synthesized using the synthetic route described in Compound 377 using (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide to afford (E)-N-(5⁵-(3-(cyclopropylsulfonyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (18.2 mg, 31% yield).

### Compound 385.

### Step 1: 4-(1-(tetrahydro-2H-pyran-2-yl)-1H-pyrazol-4-yl) morpholin-3-one

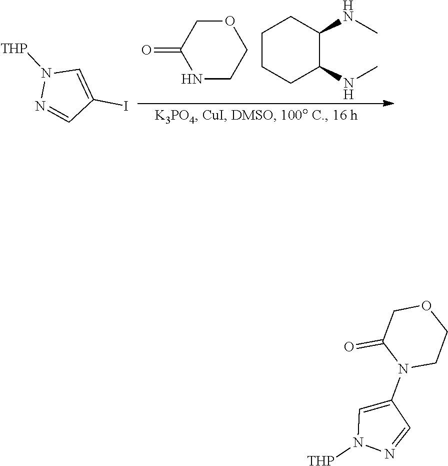

K₃PO₄ (5 g, 20 mmol) was added to the mixture of 4-iodo-1-(tetrahydro-2H-pyran-2-yl)-1H-pyrazole (2 g, 7 mmol), morpholin-3-one (1 g, 0.01 mmol), (1R,2R)—N¹, N²-dimethylcyclohexane-1,2-diamine (1 g, 7 mmol) and CuI (1 g, 7 mmol) in DMSO (20 mL) under nitrogen atmosphere. The mixture was stirred at 100° C. overnight. After cooling to room temperature, the reaction mixture was diluted with H₂O (200 mL) and extracted with EA (3×200 mL). The combined organic layers were washed with brine (1 L), dried over anhydrous Na₂SO₄, filtered and concentrated under vacuum to afford the crude product. The mixture was purified by reversed-phase flash chromatography with the following conditions: Column, C18 silica gel; mobile phase, aq. 0.1% NH₄HCO₃ in ACN, 10% to 60% gradient in 10 min; detector, UV 254 nm to afford the 4-(1-(tetrahydro-2H-pyran-2-yl)-1H-pyrazol-4-yl) morpholin-3-one (1.5 g, 80% yield). LCMS (ESI-MS) m/z=252.1 [M+H]⁺.

### Step 2: 4-(1H-pyrazol-4-yl) morpholin-3-one

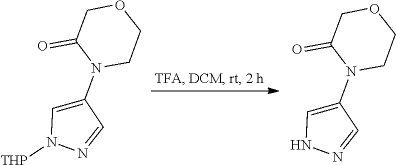

TFA (6 mL) was added to 4-(1-(tetrahydro-2H-pyran-2-yl)-1H-pyrazol-4-yl) morpholin-3-one (1.45 g, 5.77 mmol) in DCM (15 mL). The mixture was stirred at room temperature for 2 hours. The resulting mixture was concentrated under vacuum. The residue was dissolved in aq. Na₂CO₃ (20 mL). The resulting mixture was extracted with EA (3×20 mL). The combined organic layers were washed with brine (100 mL), dried over anhydrous Na₂SO₄. After filtration, the filtrate was concentrated under reduced pressure to afford 4-(1H-pyrazol-4-yl) morpholin-3-one (160 mg, 16.6% yield). LCMS (ESI-MS) m/z=168.1 [M+H]⁺.

### Step 3: Compound 385

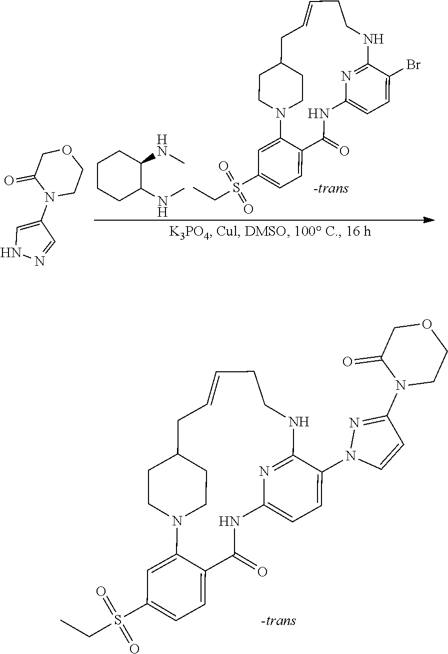

K₃PO₄ (119 mg, 0.56 mmol) was added to the mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (100 mg, 0.18 mmol), 4-(1H-pyrazol-4-yl)morpholin-3-one (160 mg, 0.95 mmol), (1R,2R)—N¹, N²-dimethylcyclohexane-1,2-diamine (2.67 mg, 0.018 mmol) and CuI (3.57 mg, 0.018 mmol) in DMSO (10 mL) under nitrogen atmosphere. The mixture was stirred at 80° C. overnight. After cooling to room temperature, the reaction mixture was diluted with H₂O (100 mL) and extracted with EA (3×100 mL). The combined organic layers were washed with brine (300 mL), dried over anhydrous Na₂SO₄, filtered and concentrated. The residue was purified by Preparative TLC, eluted with EA:PE (2:1) followed by purification by reversed-phase flash chromatography with the following conditions: Column, C18 silica gel; mobile phase, aq. 0.1% NH₄HCO₃ in ACN, 10% to 80% gradient in 10 min; detector, UV 254 nm to afford (E)-4-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl) morpholin-3-one (18.3 mg, 15.0% yield).

Compound 386 was prepared according to the procedure outlined for Compound 385 using (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide to afford (E)-N-(3-oxo-5⁵-(4-(3-oxomorpholino)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (4.5 mg, 7.1% yield).

Compounds 387 and 388 were prepared according to the procedure outlined for Compounds 310 and 311 using 3-(3-bromopyrazol-1-yl)-N2-(prop-2-en-1-yl)pyridine-2,6-diamine as the amine coupling partner in Step 3. The final products were isolated via preparative SFC with the following parameters: Column: (S, S)-Whelk-01, 5 μm, 250 mm×30 mm; Mobile Phase A: CO₂, Mobile Phase B: 1:1 MeOH/DCM (with 0.1% NH₃ (7 M in MeOH)); Flow rate: 90 mL/min; Gradient (B %): isocratic 55% B; Column Temperature (° C.): 35; Back Pressure(bar): 100; Wave Length: 220 nm; RT1 (min): 4.78; RT2 (min): 5.56; Total Elution Time (min): 10; Sample Solvent: 1:1 MeOH/DCM; Injection Volume: 0.5 mL; Number Of Runs: 121 to afford:

First eluting isomer (Compound 387): (E)-N-(5⁵-(3-bromo-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide. (2.82 g, 33% yield).

Second eluting isomer (Compound 388): (Z)—N-(5⁵-(3-bromo-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.57 g, 42% yield).

Compounds 389 and 390 were prepared according to the procedure outlined for Compounds 310 and 311 using 3-(4-bromopyrazol-1-yl)-N2-(prop-2-en-1-yl)pyridine-2,6-diamine as the amine coupling partner in Step 3. The final products were isolated via preparative SFC with the following parameters: Column: CHIRALPAK ID, 5 μm, 250 mm×30 mm; Mobile Phase A: C₀₂, Mobile Phase B: 1:1 IPA/DCM (with 0.1% NH3 (7 M in MeOH)); Flow rate: 100 mL/min; Gradient (B %): isocratic 50% B; Column Temperature (oC): 35; Back Pressure (bar): 100; Wave Length: 220 nm; RT1 (min): 3.5; RT2 (min): 4.52; Total Elution Time (min): 10; Sample Solvent: MeOH; Injection Volume: 1 mL; Number Of Runs: 21) to afford:

First eluting isomer (Compound 389): (E)-N-(5⁵-(4-bromo-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.40 g, 40% yield).

Second eluting isomer (Compound 390): (Z)—N-(5⁵-(4-bromo-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.98 g, 35% yield).

Compounds 391 and 392 were prepared according to the procedure outlined for Compound 304 using 3-(3-bromopyrazol-1-yl)-N²-(but-3-en-1-yl)pyridine-2,6-diamine as the amine coupling partner in Step 3. The final products were isolated via preparative SFC with the following parameters: Column: (R, R)-WHELK-01-Kromasil 5×25 cm, 5 μm; Mobile Phase A: CO₂, Mobile Phase B: THF--HPLC; Flow rate: 150 mL/min; Gradient (B %): isocratic 35% B; Sample Solvent: DCM:ACN=1:1; Injection Volume: 20 mL; Number Of Runs: 10) to afford:

Compound 391: (E)-N-(5⁵-(3-bromo-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (6.83 g, 56% yield).

Compound 392: (Z)—N-(55-(3-bromo-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.98 g, 35% yield).

Compounds 393 and 394 were prepared according to the procedure outlined for Compound 304 using 3-(4-bromopyrazol-1-yl)-N²-(but-3-en-1-yl)pyridine-2,6-diamine as the amine coupling partner in Step 3. The final products were isolated via preparative SFC with the following parameters: Column: CHIRALPAK IJ-3 4.6*100 mm, 3 μm; Mobile Phase B: MeOH (0.5% 2M NH₃-MeOH); Flow rate: 4 mL/min; Gradient (B %): isocratic % B to afford:

First eluting isomer (Compound 393): (E)-N-(5⁵-(4-bromo-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.71 g, yield: 57% yield).

Second eluting isomer (Compound 394): (Z)—N-(5⁵-(4-bromo-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.60 g, yield: 26% yield).

Compound 395 was prepared according to the procedure outlined in Compound 385 using (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one to afford (Z)-2⁵-(ethylsulfonyl)-1⁴-methyl-5⁵-(4-(3-oxomorpholino)-1H-pyrazol-1-yl)-11-oxa-4,6-diaza-5(2,6)-pyridina-1(1,4)-piperidina-2(1,2)-benzenacycloundecaphan-8-en-3-one (6.0 mg, 10% yield).

Compound 396 was prepared according to the procedure outlined in Compound 385 using (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one to afford (E)-2⁵-(ethylsulfonyl)-1⁴-methyl-5⁵-(4-(3-oxomorpholino)-1H-pyrazol-1-yl)-11-oxa-4,6-diaza-5(2,6)-pyridina-1(1,4)-piperidina-2(1,2)-benzenacycloundecaphan-8-en-3-one (3.1 mg, 5.3% yield).

### Compound 397.

### Step 1: (1H-pyrazol-4-yl)(pyrrolidin-1-yl)methanone

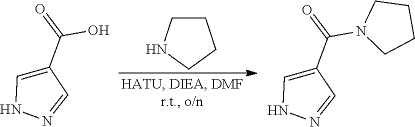 [SMILES: O=C(O)c1cn[nH]c1 ; C1CCNC1 ; O=C(c1cn[nH]c1)N1CCCC1]

DIEA (1.73 g, 13.4 mmol) was added into a mixture of 1H-pyrazole-4-carboxylic acid (500 mg, 4.46 mmol), pyrrolidine (634 mg, 8.92 mmol) and HATU (2.54 g, 6.69 mmol) in DMF (5 mL) at room temperature. The resulting mixture was stirred for overnight. The crude product was purified by reversed-phase column and eluted with ACN in water (NH₄HCO₃ 0.1%) from 0% to 50% to afford (1H-pyrazol-4-yl)(pyrrolidin-1-yl)methanone (300 mg, 38% yield).

Step 2: Compound 397

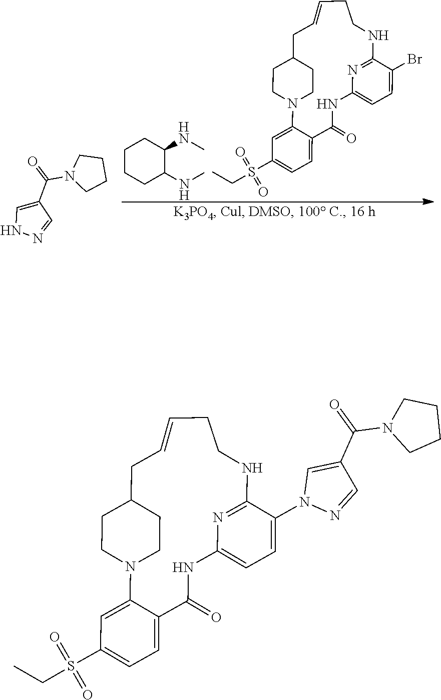

(1R,2R)—N¹,N²-dimethylcyclohexane-1,2-diamine (2 mg, 0.01 mmol) and CuI (2 mg, 0.01 mmol) were added into a mixture of (E)-5⁵-bromo-25-(ethylsulfonyl)-4,6-diaza-5(2,6)-pyridina-1(1,4)-piperidina-2(1,2)-benzenacycloundecaphan-9-en-3-one (50 mg, 0.1 mmol), (1H-pyrazol-4-yl)(pyrrolidin-1-yl)methanone (15 mg, 0.1 mmol) and K₃PO₄ (60 mg, 0.28 mmol) in DMSO (0.5 mL) under nitrogen atmosphere. The resulting mixture was stirred for overnight at 100° C. The reaction was quenched with water (5 mL) and extracted with EA (3×5 mL). The organic phase was washed with brine (15 mL), dried with Na₂SO₄, and. The residue was purified by Preparative TLC ([PE:EA][1:5]) followed by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 44% B to 74% B in 8 min; Wave Length: 254 nm/220 nm n; RT1 (min): 7.47 to afford (E)-2⁵-(ethylsulfonyl)-5⁵-(4-(pyrrolidine-1-carbonyl)-1H-pyrazol-1-yl)-4,6-diaza-5(2,6)-pyridina-1(1,4)-piperidina-2(1,2)-benzenacycloundecaphan-9-en-3-one (5.2 mg, 9.0% yield).

Compound 398 was prepared according to the procedure outlined in Compound 397 using (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide to afford (E)-N-(3-oxo-5⁵-(4-(pyrrolidine-1-carbonyl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (12.1 mg, 21% yield).

### Compound 399.

### Step 1: 4-(pyrrolidin-1-ylsulfonyl)-1H-pyrazole

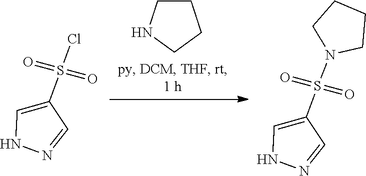 [SMILES: O=S(=O)(Cl)c1cn[nH]c1 ; C1CCNC1 ; O=S(=O)(c1cn[nH]c1)N1CCCC1]

1H-pyrazole-4-sulfonyl chloride (1 g, 6 mmol) in DCM (20 mL) was added to pyrrolidine (0.5 g, 7 mmol) in THF (10 mL). The mixture was stirred at room temperature for 1 hour then concentrated. The crude product was purified by reversed-phase flash chromatography with the following conditions: column, C18 silica gel; mobile phase, aq. 0.01% NH₄HCO₃ in ACN, 10% to 50% gradient in 10 min; detector, UV 254 nm to afford the product 4-(pyrrolidin-1-ylsulfonyl)-1H-pyrazole (300 mg, 20% yield). LCMS (ESI-MS) m/z=202.1 [M+H]⁺.

### Step 2: Compound 399

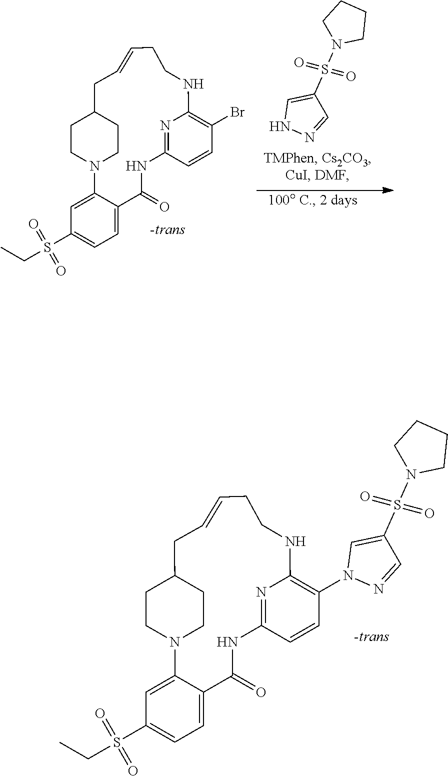 [SMILES: CCS(=O)(=O)c1ccc2c(c1)N1CCC(C/C=C/CCNc3nc(ccc3Br)NC2=O)CC1 ; O=S(=O)(c1cn[nH]c1)N1CCCC1 ; CCS(=O)(=O)c1ccc2c(c1)N1CCC(C/C=C/CCNc3nc(ccc3-n3cc(S(=O)(=O)N4CCCC4)cn3)NC2=O)CC1]

Cs₂CO₃ (55 mg, 0.17 mmol) was added to the mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (30 mg, 0.06 mmol), 4-(pyrrolidin-1-ylsulfonyl)-1H-pyrazole (34 mg, 0.17 mmol), CuI (1.1 mg, 0.06 mmol) and 3,4,7,8-Tetramethyl-1,10-diazaphenanthrene (1.3 mg, 0.06 mmol) in DMF (1 mL) under nitrogen atmosphere. The mixture was stirred at 100° C. for 48 hours. The mixture was purified by Prep-HPLC with following condition (Column: X-Bridge Prep Shield RP18 OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 45% B to72% B in 10 min; Wave Length: 254 nm/220 nm n; RT1 (min): 8.37) to afford the product (E)-2⁵-(ethylsulfonyl)-5⁵-(4-(pyrrolidin-1-ylsulfonyl)-1H-pyrazol-1-yl)-4,6-diaza-5(2,6)-pyridina-1(1,4)-piperidina-2(1,2)-benzenacycloundecaphan-9-en-3-one (9.0 mg, 24% yield).

Compound 400 was prepared using the procedure outlined in Compound 399 using (E)-5⁵-bromo-2⁵-(ethylsulfonyl)-4,6-diaza-5(2,6)-pyridina-1(1,4)-piperidina-2(1,2)-benzenacycloundecaphan-9-en-3-one (30 mg, 0.06 mmol) to afford (E)-2⁵-(ethylsulfonyl)-5⁵-(4-(pyrrolidin-1-ylsulfonyl)-1H-pyrazol-1-yl)-4,6-diaza-5(2,6)-pyridina-1(1,4)-piperidina-2(1,2)-benzenacycloundecaphan-9-en-3-one (9.0 mg, 24% yield).

### Compound 401.

### Step 1: 1-((2-(trimethylsilyl)ethoxy)methyl)-1H-pyrazole-3-carbaldehyde

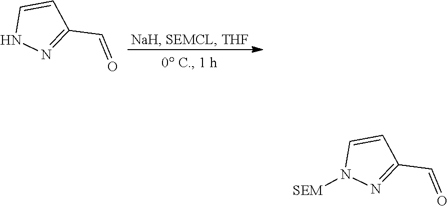 [SMILES: O=Cc1cc[nH]n1 ; C[Si](C)(C)CCOCn1ccc(C=O)n1]

To a stirred solution of 1H-pyrazole-3-carbaldehyde (5 g, 50 mmol) in THF (50 mL) was added NaH (2 g, 100 mmol) in portions at 0° C. under nitrogen atmosphere. The resulting mixture was stirred at 0° C. for additional 0.5 hours. Then added SEM-Cl (10 g, 60.00 mol) resulting mixture was stirred at 0° C. for additional 1 hour. The reaction was quenched with water at 0° C. The resulting mixture was extracted with EA (3×50 mL) then concentrated. The residue was purified by silica gel column chromatography, eluted with PE/EA (10:1) to afford 1-((2-(trimethylsilyl)ethoxy)methyl)-1H-pyrazole-3-carbaldehyde (5 g, 30% yield). LCMS (ESI-MS) m/z=227.1 [M+H]⁺.

### Step 2: (3 (1-((2-(trimethylsilyl)ethoxy)methyl)-1H-pyrazol-3-1 methanol

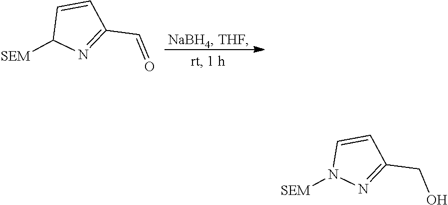 [SMILES: C[Si](C)(C)CCOCC1C=CC(C=O)=N1 ; C[Si](C)(C)CCOCn1ccc(CO)n1]

NaBH₄ (3 g, 80 mmol) was added to 1-((2-(trimethylsilyl)ethoxy)methyl)-1H-pyrazole-3-carbaldehyde (5 g, 20 mmol) in THF (50.0 mL). The mixture was stirred at room temperature for 1 hour. The resulting mixture was concentrated under vacuum. The resulting mixture was diluted with aq. NaHCO₃ (70.0 mL). The resulting mixture was extracted with EA (3×70 mL). The combined organic layers were washed with brine (70.0 mL), dried over anhydrous Na₂SO₄. After filtration, the filtrate was concentrated under reduced pressure to afford the crude product (1-((2-(trimethylsilyl)ethoxy)methyl)-1H-pyrazol-3-yl)methanol (5 g, 80% yield). LCMS (ESI-MS) m/z=229.3 [M+H]⁺.

### Step 3: 3-(chloromethyl)-1-((2-(trimethylsilyl)ethoxy)methyl)-1H-pyrazole

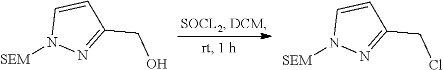 [SMILES: C[Si](C)(C)CCOCn1ccc(CO)n1 ; C[Si](C)(C)CCOCn1ccc(CCl)n1]

(1-((2-(trimethylsilyl)ethoxy)methyl)-1H-pyrazol-3-yl)methanol (5 g, 20 mmol), SOCl₂ (4 g, 30 mmol) and DCM (70.0 mL) were added to a 250 mL round-bottomed flask, and the resulting mixture stirred at room temperature for 1 hour then concentrated. The crude residue was taken up in DCM (3×50 mL), washed with sat NaHCO₃ (3×50 mL) and brine (3×50 mL), dried over anhydrous Na₂SO₄, filtered, and concentrated give 3-(chloromethyl)-1-((2-(trimethylsilyl)ethoxy)methyl)-1H-pyrazole (5 g, 70% yield). LCMS (ESI-MS) m/z=247.1 [M+H]⁺.

### Step 4: 3-((methylthio)methyl)-1-((2-(trimethylsilyl)ethoxy)methyl)-1H-pyrazole

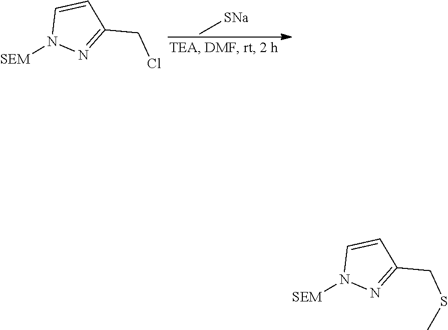

Into a solution of 3-(chloromethyl)-1-((2-(trimethylsilyl)ethoxy)methyl)-1H-pyrazole (5 g, 20 mmol) in DMF (70.0 mL) was added TEA (8 mL) and sodium methanesate, 21% aqueous solution (2 g, 20 mmol). The resulting solution was stirred at 25° C. for 2 hours. The reaction mixture was quenched with EA (3×100 mL). The organic phase was separated, washed with brine (3×100 mL), dried with anhydrous Na₂SO₄, filtered and evaporated to afford 3-((methylthio)methyl)-1-((2-(trimethylsilyl)ethoxy)methyl)-1H-pyrazole (5 g, 80% yield). LCMS (ESI-MS) m/z=259.3 [M+H]⁺.

### Step 5: 3-((methylsulfinyl)methyl)-1-((2-(trimethylsilyl)ethoxy)methyl)-1H-pyrazole

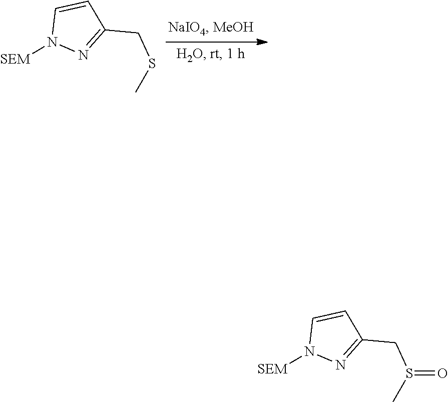 [SMILES: CSCc1ccn(COCC[Si](C)(C)C)n1 ; CS(=O)Cc1ccn(COCC[Si](C)(C)C)n1]

3-((methylthio)methyl)-1-((2-(trimethylsilyl)ethoxy)methyl)-1H-pyrazole (5 g, 20 mmol) was added to sodium metaperiodate (20 g, 100 mmol) in MeOH (140.0 mL) and H₂O (28.0 mL) at room temperature. After 1 h, the resulting mixture was concentrated under reduced pressure. The resulting residue was dissolved in water (50 mL) and the reaction mixture was extracted with EA (3×50 mL). The combined organic layers were washed with brine (150 mL), dried over anhydrous Na₂SO₄, filtered, and concentrated under reduced pressure to afford 3-((methylsulfinyl)methyl)-1-((2-(trimethylsilyl)ethoxy)methyl)-1H-pyrazole (5 g, 80% yield). LCMS (ESI-MS) m/z=275.10 [M+H]⁺.

### Step 6: 3-(1-(methylsulfinyl)cyclopropyl)-1-((2-(trimethylsilyl)ethoxy)methyl)-1H-pyrazole

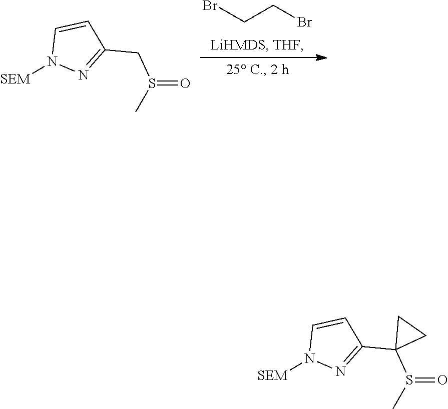

LiHMDS (40.0 mL) was added to a solution of 3-((methylsulfinyl)methyl)-1-((2-(trimethylsilyl)ethoxy)methyl)-1H-pyrazole (3 g, 10 mmol) in THF while stirring at 0° C. After 1 hour, ethylene dibromide (11.2 g, 59.5 mmol) was added and the resulting mixture was allowed to warm to 25° C. while stirring for 2 hours. After this time, the reaction was quenched by the addition of sat. NH₄Cl (aq.) (30 mL) at 0° C. and was extracted with EA (3×50 mL). The combined organic layers were washed with brine (150 mL), dried over anhydrous Na₂SO₄, filtered, concentrated under reduced pressure, and purified by reversed-phase flash column chromatography with the following conditions: column, C₁₈ silica gel; mobile phase, ACN in Water (10 mmol/L NH₄HCO₃), 30% to 70% gradient in 30 min; detector, UV 254 nm to afford 3-(1-(methylsulfinyl)cyclopropyl)-1-((2-(trimethylsilyl)ethoxy)methyl)-1H-pyrazole (580 mg, 20% yield). LCMS (ESI-MS) m/z=301.15 [M+H]⁺.

### Step 7: 3-(1-(methylsulfonyl)cyclopropyl)-1-((2-(trimethylsilyl)ethoxy)methyl)-1H-pyrazole

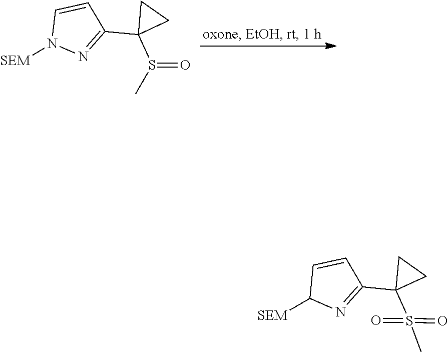 [SMILES: CS(=O)C1(c2ccn(COCC[Si](C)(C)C)n2)CC1 ; C[Si](C)(C)CCOCC1C=CC(C2(S(C)(=O)=O)CC2)=N1]

A 40 mL vial was charged with 3-(1-(methylsulfinyl)cyclopropyl)-1-((2-(trimethylsilyl)ethoxy)methyl)-1H-pyrazole (580 mg, 1.93 mmol), oxone (1.50 g, 2.44 mmol) and EtOH (7.9 mL) at room temperature. After stirring for 1 h, the reaction mixture was concentrated under reduced pressure and the resulting residue was dissolved in water (30 mL). The reaction mixture was extracted with EA (3×30 mL) and the combined organic layers were washed with brine (90 mL), dried over anhydrous Na₂SO₄, filtered, concentrated under reduced pressure, and were purified by reversed-phase flash column chromatography with the following conditions: column, C18 silica gel; mobile phase, ACN in Water (10 mmol/L NH₄HCO₃), 10% to 90% gradient in 30 min; detector, UV 254 n, to afford 3-(1-(methylsulfonyl)cyclopropyl)-1-((2-(trimethylsilyl)ethoxy)methyl)-1H-pyrazole (330 mg, 43% yield). LCMS (ESI-MS) m/z=317.10 [M+H]⁺.

### Step 8: 3-(1-(methylsulfonyl)cyclopropyl)-1H-pyrazole

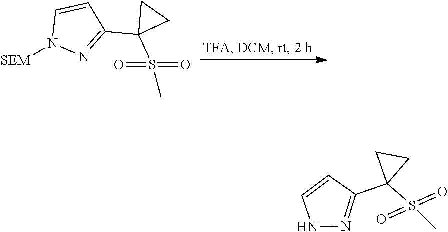 [SMILES: C[Si](C)(C)CCOCn1ccc(C2(S(C)(=O)=O)CC2)n1 ; CS(=O)(=O)C1(c2cc[nH]n2)CC1]

TFA (1.0 mL) was added to a stirred mixture of 3-(1-(methylsulfonyl)cyclopropyl)-1-((2-(trimethylsilyl)ethoxy)methyl)-1H-pyrazole (250 mg, 0.80 mmol) in DCM (1 mL). The resulting mixture was stirred at room temperature for 2 hours. The pH of the aqueous phase was adjusted to 8.0 by adding aq. NaHCO₃ solution and extracted with DCM. The combined organic layers were washed with brine, dried over Na₂SO₄, filtered, concentrated under reduced pressure, and purified by reversed-phase column and eluted with ACN in water (NH₄HCO₃ 0.1%) from 0% to 15% to afford the 3-(1-(methylsulfonyl)cyclopropyl)-1H-pyrazole (200 mg, 110% yield). LCMS (ESI-MS) m/z=187.10 [M+H]⁺.

### Step 9: Compound 401.

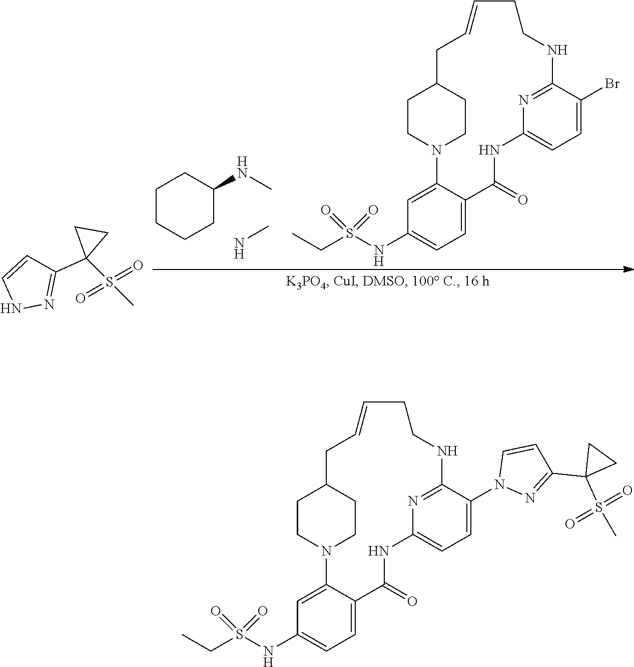

K₃PO₄ (58 mg, 0.27 mmol) was added to the mixture of 3-(1-(methylsulfonyl)cyclopropyl)-1H-pyrazole (50 mg, 0.27 mmol), (1R)—N¹,N²-dimethylcyclohexane-1,2-diamine (1.3 mg, 0.01 mmol), (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide (50 mg, 0.09 mmol) and CuI (2.0 mg, 0.01 mmol) in DMSO (1 mL) under nitrogen atmosphere. The mixture was stirred at 100° C. overnight. The crude product was purified by reverse phase flash column chromatography with the following conditions (column, C18 silica gel; mobile phase, ACN in Water (0.1% NH₃·H₂O), 80% to 20% gradient in 30 min; detector, UV 254 nm.) followed by repurification by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 20% B to 50% B in 7 min; Wave Length: 254 nm/220 nm nm; RT1 (min): 6.57) to afford (E)-N-(5⁵-(3-(1-(methylsulfonyl)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.2 mg, 8.6% yield).

### Compound 402.

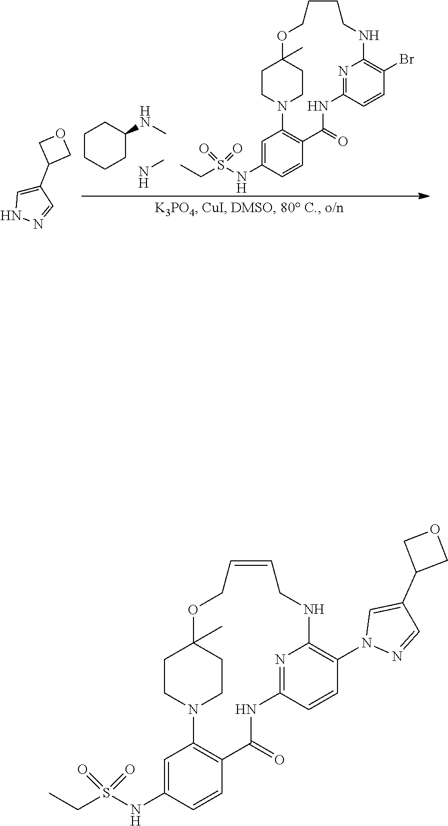

CuI (2 mg, 0.01 mmol) and (1R,2R)—N¹,N²-dimethylcyclohexane-1,2-diamine (2 mg, 0.01 mmol) were added into a mixture of (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (50 mg, 0.09 mmol), 4-(oxetan-3-yl)-1H-pyrazole (11 mg, 0.09 mmol) and K₃PO₄ (56 mg, 0.27 mmol) in DMSO (0.5 mL) under nitrogen atmosphere. The resulting mixture was stirred overnight at 80° C. After this time, the reaction was quenched with water (5 mL). The aqueous layer was extracted with EA (3×5 mL) and the organic phase was washed with brine (15 mL), dried with Na₂SO₄ and concentrated. The residue was purified by preparative TLC ([PE:FA][1:5]) followed by Prep-HPL C purification with the following conditions: Column: XBridge Prep Shield RP18 OBD C18 Column, 30*150 mm, 5 μm; Mobile Phase A: Water (10 mmol/L NH₄HCO₃), Mobile Phase B: ACN; Flow rate: 60 mL/min; Gradient (B %): 39% B to 64% B in 10 min; Wave Length: 254 nm/220 nm nm; RT 1 (min): 8.37 to afford (Z)—N-(1⁴-methyl-5⁵-(4-(oxetan-3-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (9.6 mg, 18% yield).

Compound 403 was prepared according to the procedure outlined in Compound 385 using (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide to afford (Z)—N-(1⁴-methyl-3-oxo-5⁵-(4-(3-oxomorpholino)-1H-pyrazol-1-yl)-11-oxa-4,6-diaza-5(2,6)-pyridina-1(1,4)-piperidina-2(1,2)-benzenacy-cloundecaphan-8-en-2⁵-yl)ethanesulfonamide (8.4 mg, 7.6% yield).

### Compound 404.

t-BuONa (41 mg, 0.43 mmol) was added to the mixture of (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (80 mg, 0.14 mmol), 4-(1H-pyrazol-4-yl)morpholin-3-one (160 mg, 0.95 mmol), CuI (4 mg, 0.02 mmol) and N¹,N²-bis(furan-2-ylmethyl)oxalamide (4 mg, 0.02 mmol) in DMSO (5 mL) under nitrogen atmosphere. The mixture was stirred at 100° C. overnight. After cooling to room temperature, the reaction mixture was diluted with H₂O (50 mL) and extracted with EA (3×50 mL). The combined organic layers were washed with brine (200 mL), dried over anhydrous Na₂SO₄, filtered and concentrated under vacuum to afford the crude product. The crude product purified by reversed-phase flash chromatography, with the following conditions: Column: X-Bridge Prep OBD C18 Column, 19*250 mm, 5 μm; Mobile Phase A: Water(10 mmol/L NH₄HCO₃), Mobile Phase B: MeOH; Flow rate: 25 mL/min; Gradient (B %): 55% B to 72% B in 10 min; Wave Length: 254 nm/220 nm; RT1 (min): 9.23 to afford (E)-N-(1⁴-methyl-3-oxo-5⁵-(4-(3-oxomorpholino)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (8.2 mg, 8.0% yield).

Compound 405 was prepared according to the procedure outlined in Compound 486 using (E)-N-(5⁵-bromo-3-oxo-4,6-diaza-5(2,6)-pyridina-1(1,4)-piperidina-2(1,2)-benzenacycloundecaphan-9-en-2⁵-yl)ethanesulfonamide to afford (E)-N-(3-oxo-5⁵-(4-(2-oxooxazolidin-3-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (8.8 mg, 26% yield).

Compound 406 was prepared according to the procedure used to prepare Compound 589 using Intermediate AA and 4-bromo-2-methylbutan-2-ol to afford (E)-5⁵-bromo-2⁵-((3-hydroxy-3-methylbutyl)sulfonyl)-4,6-diaza-5(2,6)-pyridina-1(1,4)-piperidina-2(1,2)-benzenacycloundecaphan-9-en-3-one (2.4 mg, 6% yield).

Compound 407 was prepared according to the procedure used to prepare Compound 589 using Intermediate AA and 3-bromo-1-propanol to afford (E)-5⁵-bromo-2⁵-((3-hydroxypropyl)sulfonyl)-4,6-diaza-5(2,6)-pyridina-1(1,4)-piperidina-2(1,2)-benzenacycloundecaphan-9-en-3-one (1.4 mg, 3.7% yield).

Compound 408 was prepared according to the procedure used to prepare Compound 589 using Intermediate AA and 2-bromoethyl methyl ether to afford (E)-5⁵-bromo-2⁵-((2-methoxyethyl)sulfonyl)-4,6-diaza-5(2,6)-pyridina-1(1,4)-piperidina-2(1,2)-benzenacycloundecaphan-9-en-3-one (1.7 mg, 5.4% yield).

Compound 409 was prepared according to General Procedure 1 using Compound 391 and 2-thia-6-azaspiro[3.4]octane 2,2-dioxide hydrochloride to afford (E)-N-(5⁵-(3-(2,2-dioxido-2-thia-6-azaspiro[3.4]octan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (5.6 mg, 32.4% yield).

Compound 410 was prepared according to General Procedure 1 using Compound 391 and (S)-2-(3-pyrrolidinyl)-2-propanol to afford (S,E)-N-(5⁵-(3-(3-(2-hydroxypropan-2-yl)pyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (8.9 mg, 53.5% yield).

Compound 411 was prepared according to General Procedure 1 using Compound 391 and 1-{2,7-diazaspiro[4.4]nonan-2-yl}ethanone to afford (E)-N-(5⁵-(3-(7-acetyl-2,7-diazaspiro[4.4]nonan-2-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (3.3 mg, 18.5% yield).

Compound 412 was prepared according to General Procedure 1 using Compound 391 and (4-methoxypiperidin-4-yl)methanol to afford (E)-N-(55-(3-(4-(hydroxymethyl)-4-methoxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (10.1 mg, 59.4% yield).

Compound 413 was prepared according to General Procedure 1 using Compound 391 and (R)-piperidin-3-ol to afford (R,E)-N-(5⁵-(3-(3-hydroxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (13 mg, 82% yield).

Compound 414 was prepared according to General Procedure 1 using Compound 391 and 3,3,4,4-tetrafluoropyrrolidine hydrochloride to afford (E)-N-(3-oxo-5⁵-(3-(3,3,4,4-tetrafluoropyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2.8 mg, 16.5% yield).

Compound 415 was prepared according to General Procedure 1 using Compound 391 and (S)-3-methoxypiperidine to afford (S,E)-N-(5⁵-(3-(3-methoxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.6 mg, 9.8% yield).

Compound 416 was prepared according to General Procedure 1 using Compound 391 and N-methylcyclobutanamine hydrochloride to afford (E)-N-(5⁵-(3-(cyclobutyl(methyl)amino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.3 mg, 21.3% yield).

Compound 417 was prepared according to General Procedure 1 using Compound 391 and 3-methylsulfonyl-azetidine hydrochloride to afford (E)-N-(5⁵-(3-(3-(methylsulfonyl)azetidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4 mg, 24% yield).

Compound 418 was prepared according to General Procedure 1 using Compound 391 and 2-oxa-6-azaspiro[3.3]heptane to afford (E)-N-(5⁵-(3-(2-oxa-6-azaspiro[3.3]heptan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (8.8 mg, 55.5% yield).

Compound 419 was prepared according to General Procedure 1 using Compound 391 and 4,4-difluoropiperidine hydrochloride to afford (E)-N-(5⁵-(3-(4,4-difluoropiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.6 mg, 16.1% yield).

Compound 420 was prepared according to General Procedure 1 using Compound 391 and (3R,4R)-3,4-dimethoxypyrrolidine hydrochloride to afford N—((E)-55-(3-((3R,4R)-3,4-dimethoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.3 mg, 13.7% yield).

Compound 421 was prepared according to General Procedure 1 using Compound 391 and (S)-3-methoxypyrrolidine to afford (S,E)-N-(5⁵-(3-(3-methoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (10.9 mg, 68.6% yield).

Compound 422 was prepared according to General Procedure 1 using Compound 391 and (R)-3-methoxypyrrolidine to afford (R,E)-N-(55-(3-(3-methoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (9.5 mg, 59.6% yield).

Compound 423 was prepared according to General Procedure 1 using Compound 391 and (3S,4S)-3-fluoro-4-methoxypyrrolidine hydrochloride to afford N—((E)-5⁵-(3-((3S,4S)-3-fluoro-4-methoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.6 mg, 15.9% yield).

Compound 424 was prepared according to General Procedure 1 using Compound 391 and 1,2,3,4-tetrahydropyrrolo[1,2-a]pyrazine to afford (E)-N-(55-(3-(3,4-dihydropyrrolo[1,2-a]pyrazin-2(1H)-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.9 mg, 11.7% yield).

Compound 425 was prepared according to General Procedure 1 using Compound 391 and thiomorpholine 1,1-dioxide hydrochloride to afford (E)-N-(55-(3-(1,1-dioxidothiomorpholino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 12.3% yield).

Compound 426 was prepared according to General Procedure 1 using Compound 391 and (R)-3-hydroxypyrrolidine hydrochloride to afford (R,E)-N-(5⁵-(3-(3-hydroxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.3 mg, 27.4% yield).

Compound 427 was prepared according to General Procedure 1 using Compound 391 and (S)-pyrrolidin-3-ol to afford S,E)-N-(5⁵-(3-(3-hydroxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.7 mg, 37% yield).

Compound 428 was prepared according to General Procedure 1 using Compound 391 and 3,3-difluoropyrrolidine hydrochloride to afford (E)-N-(55-(3-(3,3-difluoropyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.6 mg, 28.8% yield).

Compound 429 was prepared according to General Procedure 1 using Compound 391 and 2-oxa-7-azaspiro[4.4]nonane to afford (E)-N-(55-(3-(2-oxa-7-azaspiro[4.4]nonan-7-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (13.8 mg, 83.3% yield).

Compound 430 was prepared according to General Procedure 1 using Compound 391 and (3R)-3-(difluoromethyl)pyrrolidine hydrochloride to afford (R,E)-N-(5⁵-(3-(3-(difluoromethyl)pyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.4 mg, 14.6% yield).

Compound 431 was prepared according to General Procedure 1 using Compound 391 and thiomorpholine-1-oxide hydrochloride to afford (E)-N-(5⁵-(3-(1-oxidothiomorpholino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (9.7 mg, 59.7% yield).

Compound 432 was prepared according to General Procedure 1 using Compound 391 and 1-oxa-6-azaspiro[3.4]octane to afford (E)-N-(55-(3-(1-oxa-6-azaspiro[3.4]octan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (14.9 mg, 92% yield).

Compound 433 was prepared according to General Procedure 1 using Compound 391 and 4,4-dimethyl-1,4-azasilinane hydrochloride to afford (E)-N-(5⁵-(3-(4,4-dimethyl-1,4-azasilinan-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1 mg, 6.3% yield).

Compound 434 was prepared according to General Procedure 1 using Compound 391 and 2-methyl-2,6-diazaspiro[3.4]octane dihydrochloride (E)-N-(5⁵-(3-(2-methyl-2,6-diazaspiro[3.4]octan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide to afford (0.8 mg, 4.8% yield).

Compound 435 was prepared according to General Procedure 1 using Compound 391 and (R)-pyrrolidine-3-carbonitrile hydrochloride to afford (R,E)-N-(5⁵-(3-(3-cyanopyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.7 mg, 23.7% yield).

Compound 436 was prepared according to General Procedure 1 using Compound 391 and using potassium (piperidin-1-yl)methyltrifluoroborate to afford (E)-N-(3-oxo-55-(3-(piperidin-1-ylmethyl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (1.8 mg, 11.5% yield).

Compound 437 was prepared according to General Procedure 1 using Compound 391 and to afford (E)-N-(3-oxo-55-(3-(pyrrolidin-1-ylmethyl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (19 mg, 100% yield).

Compound 438 was prepared according to General Procedure 1 using Compound 391 and potassium (morpholin-4-yl)methyltrifluoroborate to afford (E)-N-(5⁵-(3-(morpholinomethyl)-1H-pyrazol-1-yl)-3-oxo-4,6-diaza-5(2,6)-pyridina-1(1,4)-piperidina-2(1,2)-benzenacycloundecaphan-9-en-25-yl)ethanesulfonamide (17.1 mg, 91.1% yield).

Compound 439 was prepared according to the procedure outlined in General Procedure 1 using Compound 392 and (morpholinium-4-ylmethyl)trifluoroborate internal salt to afford (21.3 mg, 100% yield).

Compound 440 was prepared according to General Procedure 1 using Compound 392 and potassium trifluoro[(pyrrolidin-1-yl)methyl]borate to afford (Z)—N-(3-oxo-5⁵-(3-(pyrrolidin-1-ylmethyl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (17.7 mg, 96.8% yield).

Compound 441 was prepared according to General Procedure 1 using Compound 392 and 2,7-diazaspiro[4.4]nonan-1-one hydrochloride to afford (Z)—N-(3-oxo-5⁵-(3-(6-oxo-2,7-diazaspiro[4.4]nonan-2-yl)-1H-pyrazol-1-yl)-4,6-diaza-5(2,6)-pyridina-1(1,4)-piperidina-2(1,2)-benzenacycloundecaphanen-2⁵-yl)ethanesulfonamide (0.6 mg, 3.5% yield).

Compound 442 was prepared according to General Procedure 1 using Compound 392 and thiomorpholine-1-oxide hydrochloride to afford (Z)—N-(55-(3-(1-oxidothiomorpholino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 12.8% yield).

Compound 443 was prepared according to General Procedure 1 using Compound 392 and (S)-3-hydroxypyrrolidine hydrochloride to afford (S,Z)—N-(5⁵-(3-(3-hydroxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.6 mg, 10.2% yield).

Compound 444 was prepared according to General Procedure 1 using Compound 392 and (S)-2-(3-pyrrolidinyl)-2-propanol to afford (S,Z)—N-(5⁵-(3-(3-(2-hydroxypropan-2-yl)pyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.3 mg, 7.7% yield).

### Compound 445.

### Step 1: 2-(1-((2-(trimethylsilyl) ethoxy) methyl)-1H-pyrazol-4-yl) acetonitrile

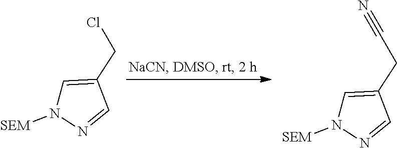 [SMILES: C[Si](C)(C)CCOCn1cc(CCl)cn1 ; C[Si](C)(C)CCOCn1cc(CC#N)cn1]

Sodium cyanide (1.0 equiv) was added to the mixture of 4-(chloromethyl)-1-((2-(trimethylsilyl) ethoxy) methyl)-1H-pyrazole (1.0 equiv) in DMSO (8 V). The mixture was stirred at 25° C. for 1 hour. The reaction was poured into water (500 mL) at room temperature. The resulting mixture was extracted with EA (3×50 V). The combined organic layers were washed with brine (200 V), dried over anhydrous Na₂SO₄. After filtration, the filtrate was concentrated under reduced pressure. The residue was purified by silica gel column chromatography, eluted with THF:PE (1:9) to afford 2-(1-((2-(trimethylsilyl)ethoxy)methyl)-1H-pyrazol-4-yl)acetonitrile (56% yield).

### Step 2: 1-(1-((2-(trimethylsilyl) ethoxy) methyl)-1H-pyrazol-4-yl) cyclopropane-1-carbonitrile

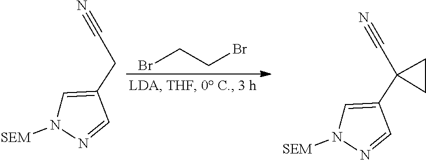

A solution of 2-(1-((2-(trimethylsilyl) ethoxy) methyl)-1H-pyrazol-4-yl) acetonitrile (1.0 equiv) in THF (10 V) was added to 2.5 M LDA in hexane (2.0 equiv) at 0° C. under a nitrogen atmosphere. The mixture was stirred at 0° C. for 1 hours. Then 1,2-dibromoethane (2.0 equiv) was added to the reaction mixture and stirred at room temperature for 3 hours. The reaction was poured into water (20 V) at room temperature and the resulting mixture was extracted with EA (3×20 V). The combined organic layers were washed with brine (40 V), dried over anhydrous Na₂SO₄. After filtration, the filtrate was concentrated under reduced pressure. The residue was purified by silica gel column chromatography, eluted with THF:PE (1:9) to afford 1-(1-((2-(trimethylsilyl) ethoxy) methyl)-1H-pyrazol-4-yl) cyclopropane-1-carbonitrile (41% yield).

### Step 3: 1-(1H-pyrazol-4-yl) cyclopropane-1-carbonitrile

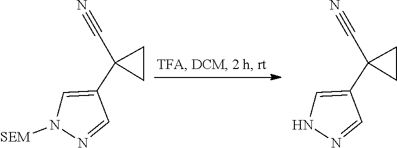 [SMILES: C[Si](C)(C)CCOCn1cc(C2(C#N)CC2)cn1 ; N#CC1(c2cn[nH]c2)CC1]

TFA (5 V) was added to 1-(1-((2-(trimethylsilyl) ethoxy) methyl)-1H-pyrazol-4-yl) cyclopropane-1-carbonitrile (1.0 equiv) in DCM (10 V). The mixture was stirred at room temperature for 2 hours. The resulting mixture was concentrated under vacuum and the resulting residue was dissolved in aq. NaHCO₃ (15 mL). The mixture was extracted with EA (3×10 V) and the combined organic layers were washed with brine (20 V), dried over anhydrous Na₂SO₄, filtered, and concentrated under reduced pressure to afford 1-(1H-pyrazol-4-yl) cyclopropane-1-carbonitrile (crude). LCMS (ESI-MS) m/z=134.1[M+H]⁺

### Step 4: Compound 445

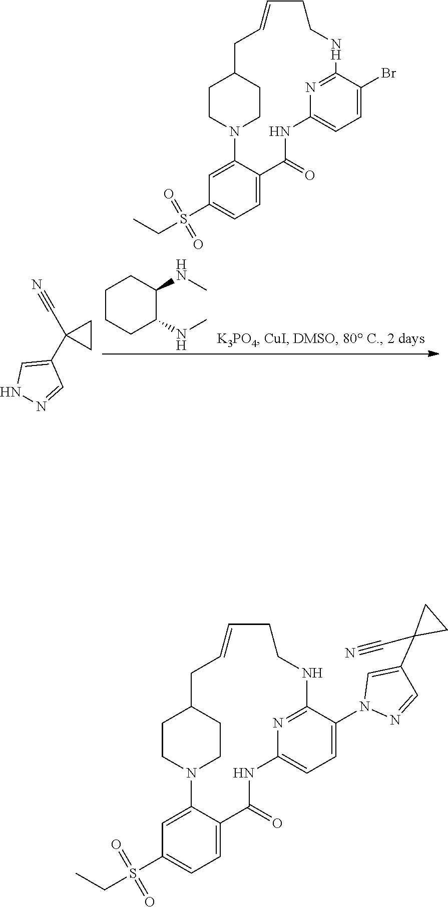

K₃PO₄ (6 equiv) was added to the mixture of 1-(1H-pyrazol-4-yl)cyclopropane-1-carbonitrile (6 equiv), (1R,2R)—N¹,N²-dimethylcyclohexane-1,2-diamine (0.10 equiv), (E)-5⁵-bromo-2⁵-(ethylsulfonyl)-4,6-diaza-5(2,6)-pyridina-1(1,4)-piperidina-2(1,2)-benzenacycloundecaphan-9-en-3-one (1.0 equiv) and CuI (0.10 equiv) in DMSO (30 V) under nitrogen atmosphere. The mixture was stirred at 80° C. for 2 days. The mixture was purified by Prep-HPLC with following condition (Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 18% B to 48% B in 10 min; Wave Length: 254 nm/220 nm n; RT1 (min): 8.29) to afford the product (E)-1-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl)cyclopropane-1-carbonitrile (7.1% yield).

Compound 446 was prepared according to General Procedure 1 using Compound 392 and 1-methylpiperazine to afford (Z)—N-(5⁵-(3-(4-methylpiperazin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (6.8 mg, 36.4% yield).

Compound 447 was prepared according to General Procedure 1 using Compound 392 and 1-{2,7-diazaspiro[4.4]nonan-2-yl}ethanone to afford (Z)—N-(55-(3-(7-acetyl-2,7-diazaspiro[4.4]nonan-2-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (5.3 mg, 30.5% yield).

Compound 448 was prepared according to General Procedure 1 using Compound 392 and (4-methoxypiperidin-4-yl)methanol to afford (Z)—N-(55-(3-(4-(hydroxymethyl)-4-methoxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.3 mg, 26.6% yield).

Compound 449 was prepared according to General Procedure 1 using Compound 392 and 4-oxa-7-azaspiro[2.5]octane hydrochloride to afford (Z)—N-(5⁵-(3-(4-oxa-7-azaspiro[2.5]octan-7-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (1.3 mg, 6.9% yield).

Compound 450 was prepared according to General Procedure 1 using Compound 392 and (3S,4S)-4-fluoropyrrolidin-3-ol hydrochloride to afford N—((Z)-55-(3-((3S,4S)-3-fluoro-4-hydroxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.3 mg, 6.8% yield).

Compound 451 was prepared according to General Procedure 1 using Compound 392 and 3,3-difluoropyrrolidine to afford (Z)—N-(55-(3-(3,3-difluoropyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.9 mg, 31.4% yield).

Compound 452 was prepared according to General Procedure 1 using Compound 392 and (4-fluoropiperidin-4-yl)methanol hydrochloride to afford (Z)—N-(5⁵-(3-(4-fluoro-4-(hydroxymethyl)piperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.9 mg, 17.4% yield).

Compound 453 was prepared according to General Procedure 1 using Compound 392 and 1-(4-piperidinyl)cyclopropanol hydrochloride to afford (Z)—N-(5⁵-(3-(4-(1-hydroxycyclopropyl)piperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.1 mg, 15.7% yield).

Compound 454 was prepared according to General Procedure 1 using Compound 392 and 3-oxa-8-azabicyclo[3.2.1]octane hydrochloride to afford (Z)—N-(5⁵-(3-(3-oxa-8-azabicyclo[3.2.1]octan-8-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.6 mg, 8.6% yield).

Compound 455 was prepared according to General Procedure 1 using Compound 392 and 2-oxa-6-azaspiro[3.3]heptane to afford (Z)—N-(55-(3-(2-oxa-6-azaspiro[3.3]heptan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (7 mg, 44.1% yield).

Compound 456 was prepared according to General Procedure 1 using Compound 392 and (S)-3-(methylsulfonyl)pyrrolidine to afford (S,Z)—N-(5⁵-(3-(3-(methylsulfonyl)pyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.2 mg, 24.4% yield).

Compound 457 was prepared according to General Procedure 1 using Compound 392 and 2-oxa-7-azaspiro[4.4]nonane to afford (Z)—N-(5⁵-(3-(2-oxa-7-azaspiro[4.4]nonan-7-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (1.9 mg, 9.9% yield).

Compound 458 was prepared according to General Procedure 1 using Compound 392 and (S)-3-methoxypiperidine to afford (S,Z)—N-(55-(3-(3-methoxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.6 mg, 24.1% yield).

Compound 459 was prepared according to General Procedure 1 using Compound 392 and 2-methyl-2,6-diazaspiro[3.4]octane dihydrochloride to afford (Z)—N-(5⁵-(3-(2-methyl-2,6-diazaspiro[3.4]octan-6-yl)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (0.6 mg, 3.4% yield).

Compound 460 was prepared according to General Procedure 1 using Compound 392 and N-methylcyclobutanamine hydrochloride to afford (Z)—N-(5⁵-(3-(cyclobutyl(methyl)amino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.4 mg, 7.4% yield).

Compound 461 was prepared according to General Procedure 1 using Compound 392 and 2-oxa-5-azabicyclo[2.2.1]heptane to afford (Z)—N-(5⁵-(3-(2-oxa-5-azabicyclo[2.2.1]heptan-5-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.9 mg, 26.4% yield).

### Compound 462.

### Step 1: (E)-5⁵-(3-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one

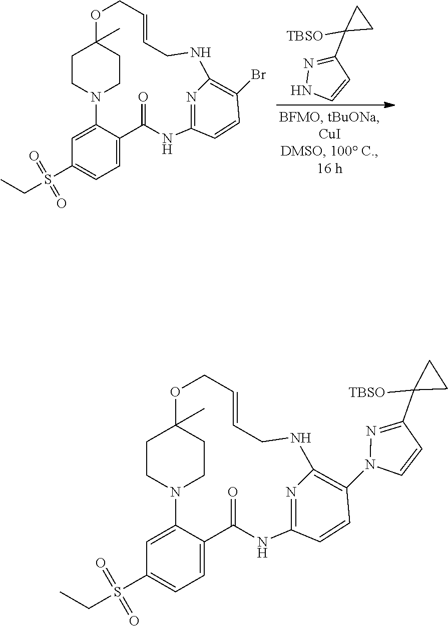 [SMILES: CCS(=O)(=O)c1ccc2c(c1)N1CCC(C)(CC1)OC/C=C/CNc1nc(ccc1Br)NC2=O ; CC(C)(C)[Si](C)(C)OC1(c2cc[nH]n2)CC1 ; CCS(=O)(=O)c1ccc2c(c1)N1CCC(C)(CC1)OC/C=C/CNc1nc(ccc1-n1ccc(C3(O[Si](C)(C)C(C)(C)C)CC3)n1)NC2=O]

Under a nitrogen atmosphere, a solution of (E)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (1.0 equiv) in DMSO (6 V) was added to 3-(1-((tert-butyldimethylsilyl)oxy)cyclopropyl)-1H-pyrazole (3.0 equiv), sodium 2-methylpropan-2-olate (4.0 equiv), N¹,N²-bis(furan-2-ylmethyl)oxalamide (0.10 equiv and CuI (0.10 equiv). After stirring for 16 hours at 100° C., the mixture was allowed to cool to room temperature, was concentrated under reduced pressure, and the resulting residue was purified by reversed-phase flash column chromatography with the following conditions: column, C18 silica gel; mobile phase, ACN in Water (0.1% NH₃·H₂O), 10% to 80% gradient in 40 min; detector, UV 254 nm. to afford (E)-5⁵-(3-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (25% yield). LCMS (ESI-MS) m/z=707.4 [M+H]⁺.

Step 2: Compound 462: 1M TBAF in THF (2.0 equiv) was added to (E)-5⁵-(3-(1-((tert-butyldimethylsilyl)oxy)cyclopropyl)-1H-pyrazol-1-yl)-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (1.0 equiv) in THF (40 V) at 25° C. The resulting mixture was extracted with EA (3×10 V). The combined organic layers were washed with water (3×5 V), dried over anhydrous Na₂SO₄, filtered, concentrated under reduced pressure, and the resulting residue was purified by silica gel column chromatography (eluted with PE/EA (1:2)) to afford the crude product. This material was then was purified by Prep-HPLC with the following conditions (Column: XBridge Prep Shield RP18 OBD C18 Column, 30*150 mm, 5 μm; Mobile Phase A: Water (10 mmol/L NH₄HCO₃), Mobile Phase B: ACN; Flow rate: 60 mL/min; Gradient (B %): 36% B to 66% B in 10 min; Wave Length: 254 nm/220 nm n; RT1 (min): 7.6) to afford (E)-2⁵-(ethylsulfonyl)-5⁵-(3-(1-hydroxycyclopropyl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (14% yield).

### Compound 463.

### Step 1: (E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one

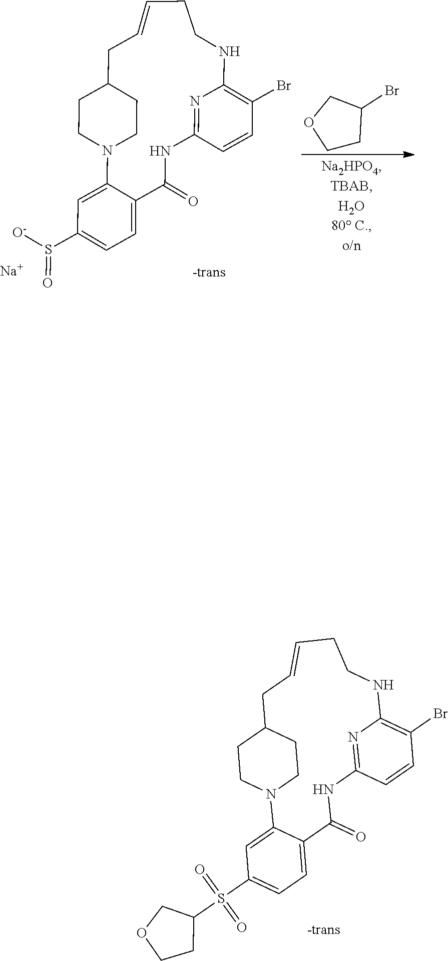 [SMILES: [Na+] ; O=C1Nc2ccc(Br)c(n2)NCC/C=C/CC2CCN(CC2)c2cc(S(=O)[O-])ccc21 ; BrC1CCOC1 ; O=C1Nc2ccc(Br)c(n2)NCC/C=C/CC2CCN(CC2)c2cc(S(=O)(=O)C3CCOC3)ccc21]

Na₂HPO₄ (26.9 mg, 0.19 mmol) and TBAB (61.1 mg, 0.19 mmol) were added to a mixture of sodium (E)-5⁵-bromo-3-oxo-4,6-diaza-5(2,6)-pyridina-1(1,4)-piperidina-2(1,2)-benzenacycloundecaphan-9-ene-2⁵-sulfinate (Intermediate AA, 100 mg, 0.19 mmol) and 3-bromotetrahydrofuran (28.6 mg, 0.19 mmol) in H₂O (1 mL) at room temperature under nitrogen atmosphere. The resulting mixture was stirred for overnight at 80° C. under a nitrogen atmosphere. After this time, the reaction mixture was diluted with water (3 mL) and extracted with EA (3×3 mL). The combined organic layers were washed with brine (10 mL), dried over anhydrous Na₂SO₄, filtered, concentrated under reduced pressure, and purified iva flash column chromatography (Silica-CS (20 g) and eluting with EA in PE from 0% to 40%) to afford (E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one (60 mg, 52% yield). LCMS (ESI-MS) m/z=575.1 [M+H]⁺.

### Step 2: (R,E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one

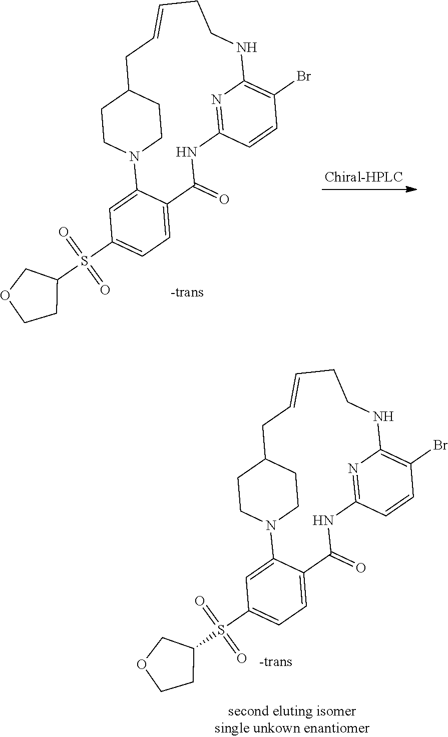 [SMILES: O=C1Nc2ccc(Br)c(n2)NCC/C=C/CC2CCN(CC2)c2cc(S(=O)(=O)C3CCOC3)ccc21 ; O=C1Nc2ccc(Br)c(n2)NCC/C=C/CC2CCN(CC2)c2cc(S(=O)(=O)[C@@H]3CCOC3)ccc21]

(E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one (60 mg, 0.10 mmol) was separated by preparative Chiral HPLC with the following condition: Column: CHIRALPAK IM, 5 μm, 250 mm×20 mm; Mobile Phase A: Hex (with 0.2% FA), Mobile Phase B: EtOH:DCM=1:1; Flow rate: 20 mL/min; Gradient (B %) 50% B; Wave Length: 254/220 n; RT1 (min): 7.69; RT2 (min): 9.61; Sample Solvent: EtOH--HPLC; Injection Volume: 0.9 mL; Number Of Runs: 4; Single injection run time (min): 12 to afford (R,E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one (35 mg, 56% yield). LCMS (ESI-MS) m/z=575.1 [M+H]⁺.

### Step 3: (R,E)-5⁵-(4-(1-((tert-butyldimethylsilyl)oxy)cyclopropyl)-1H-pyrazol-1-yl)-2⁵-((tetrahydrofuran-3-yl)sulfonyl)-4,6-diaza-5(2,6)-pyridina-1(1,4)-piperidina-2(1,2)-benzenacycloundecaphan-9-en-3-one

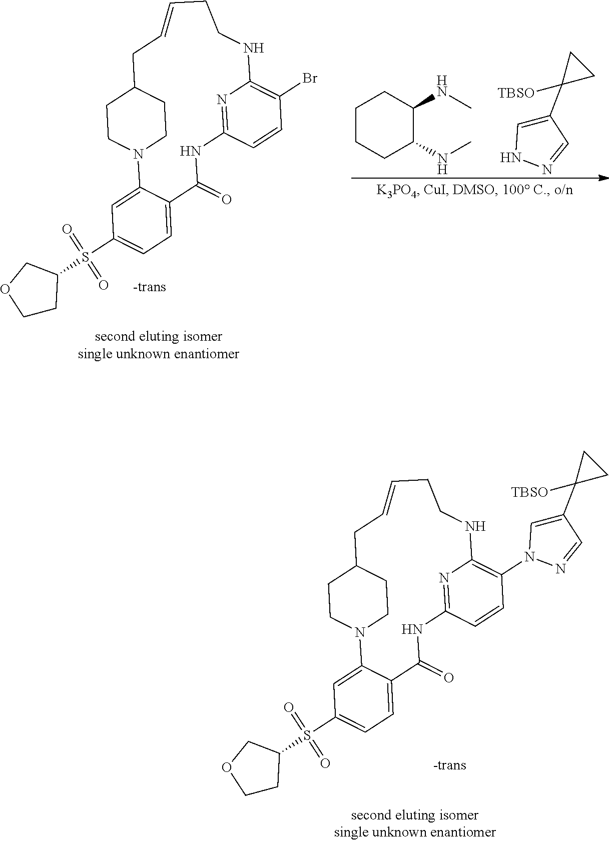

(1R,2R)—N¹,N²-dimethylcyclohexane-1,2-diamine (1 mg, 0.01 mmol) and CuI (2 mg, 0.01 mmol) were added to a mixture of (R,E)-5⁵-bromo-2⁵-((tetrahydrofuran-3-yl)sulfonyl)-4,6-diaza-5(2,6)-pyridina-1(1,4)-piperidina-2(1,2)-benzenacycloundecaphan-9-en-3-one (35 mg, 0.06 mmol), 4-(1-((tert-butyldimethylsilyl)oxy)cyclopropyl)-1H-pyrazole (29 mg, 0.12 mmol) and K₃PO₄ (39 mg, 0.18 mmol) in DMSO (0.5 mL) at room temperature under nitrogen atmosphere. The resulting mixture was stirred overnight at 100° C. After this time, the reaction mixture was cooled to room temperature, was diluted with water (5 mL), and was extracted with EA (3×5 mL). The combined organic layers were washed with brine (15 mL), dried over anhydrous Na₂SO₄, filtered, concentrated under reduced pressure, and the resulting residue was purified by preparative TLC (PE:EA 1:1) to afford the product (R,E)-5⁵-(4-(1-((tert-butyldimethylsilyl)oxy)cyclopropyl)-1H-pyrazol-1-yl)-2⁵-((tetrahydrofuran-3-yl)sulfonyl)-4,6-diaza-5(2,6)-pyridina-1(1,4)-piperidina-2(1,2)-benzenacycloundecaphan-9-en-3-one (10 mg, 21% yield). LCMS (ESI-MS) m/z=733.3 [M+H]⁺.

Step 4: Compound 463: 1M TBAF in THF (0.1 mL) was added to the stirred mixture of (R,E)-5⁵-(4-(1-((tert-butyldimethylsilyl)oxy)cyclopropyl)-1H-pyrazol-1-yl)-2⁵-((tetrahydrofuran-3-yl)sulfonyl)-4,6-diaza-5(2,6)-pyridina-1(1,4)-piperidina-2(1,2)-benzenacycloundecaphan-9-en-3-one (10 mg, 0.01 mmol) in THF (0.5 mL), then the resulting mixture was stirred for 1 hour at room temperature. After this time, the reaction mixture was diluted with NH₄Cl (3×3 mL) and extracted with EA (3×3 mL). The combined organic layers were washed with brine (9 mL), dried over anhydrous Na₂SO₄, filtered, and concentrated under reduced pressure to afford the crude product. This residue was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 45% B to 75% B in 8 min; Wave Length: 254 nm/220 nm nm; RT1 (min): 6.98 to afford (R,E)-5⁵-(4-(1-hydroxycyclopropyl)-1H-pyrazol-1-yl)-2⁵-((tetrahydrofuran-3-yl)sulfonyl)-4,6-diaza-5(2,6)-pyridina-1(1,4)-piperidina-2(1,2)-benzenacycloundecaphan-9-en-3-one (1.4 mg, 17% yield).

### Compound 464.

### Step 1: (Z)-55-(4-(1-((tert-butyldimethylsilyl)oxy)cyclopropyl)-1H-pyrazol-1-yl)-2⁵-((2-hydroxyethyl)sulfonyl)-14-methyl-11-oxa-4,6-diaza-5(2,6)-pyridina-1(1,4)-piperidina-2(1,2)-benzenacycloundecaphan-8-en-3-one

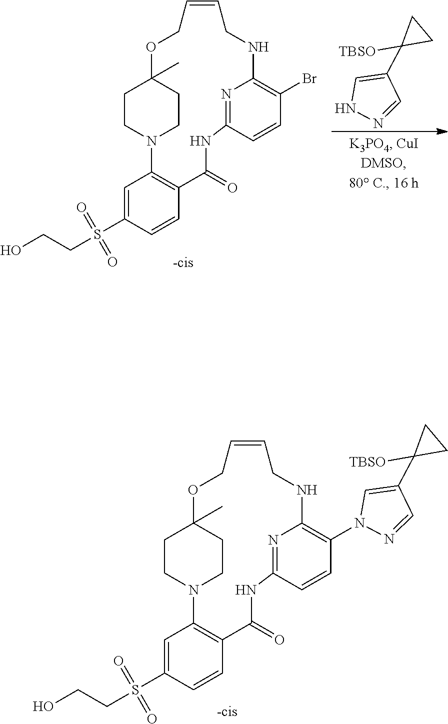 [SMILES: CC12CCN(CC1)c1cc(S(=O)(=O)CCO)ccc1C(=O)Nc1ccc(Br)c(n1)NC/C=C\CO2 ; CC(C)(C)[Si](C)(C)OC1(c2cn[nH]c2)CC1 ; CC12CCN(CC1)c1cc(S(=O)(=O)CCO)ccc1C(=O)Nc1ccc(-n3cc(C4(O[Si](C)(C)C(C)(C)C)CC4)cn3)c(n1)NC/C=C\CO2]

(1R,2R)—N¹,N²-dimethylcyclohexane-1,2-diamine (2.0 mg, 0.01 mmol) and CuI (3.0 mg, 0.01 mmol) were added to a mixture of (Z)-5⁵-bromo-2⁵-((2-hydroxyethyl)sulfonyl)-1⁴-methyl-11-oxa-4,6-diaza-5(2,6)-pyridina-1(1,4)-piperidina-2(1,2)-benzenacycloundecaphan-8-en-3-one (70 mg, 0.12 mmol, Step 5 of Compound 229), 4-(1-((tert-butyldimethylsilyl)oxy)cyclopropyl)-1H-pyrazole (89 mg, 0.37 mmol) and K₃PO₄ (79 mg, 0.37 mmol) in DMSO (4 mL) at room temperature under nitrogen atmosphere. The resulting mixture was stirred for 16 hours at 80° C. under nitrogen atmosphere. After cooling to room temperature, the reaction mixture was diluted with H₂O (10 mL) and extracted with EA (3×10 mL). The combined organic layers were washed with brine (30 mL), dried over anhydrous Na₂SO₄, filtered and concentrated under vacuum to afford the crude product. The crude product was purified by reversed-phase column (90 g) and eluted with ACN in water (NH₄HCO₃ 0.1%) from 0% to 80%. Fractions with desired mass signal were combined and concentrated under vacuum to afford (Z)-5⁵-(4-(1-((tert-butyldimethylsilyl)oxy)cyclopropyl)-1H-pyrazol-1-yl)-2⁵-((2-hydroxyethyl)sulfonyl)-1⁴-methyl-11-oxa-4,6-diaza-5(2,6)-pyridina-1(1,4)-piperidina-2(1,2)-benzenacycloundecaphan-8-en-3-one (55 mg, 49% yield). LCMS (ESI-MS) m/z=723.4 [M+H]⁺.

Step 2: Compound 464: 2M TBAF in THF (0.03 mL) was added to (Z)-5⁵-(4-(1-((tert-butyldimethylsilyl)oxy)cyclopropyl)-1H-pyrazol-1-yl)-2⁵-((2-hydroxyethyl)sulfonyl)-1⁴-methyl-11-oxa-4,6-diaza-5(2,6)-pyridina-1(1,4)-piperidina-2(1,2)-benzenacycloundecaphan-8-en-3-one (50 mg, 0.07 mmol) in THF (2 mL). The mixture was stirred at room temperature for 1 hour. The reaction was then poured into EA (10 mL) and extracted with aq. NH₄Cl (3×10 mL). The combined organic layers were washed with aq. NH₄Cl (15 mL), dried over anhydrous Na₂SO₄. After filtration, the residue was purified by reversed-phase flash chromatography with the following conditions: column, C18 silica gel; mobile phase, aq. 0.01% TFA in ACN, 10% to 90% gradient in 10 min; detector, UV 254 nm to afford the crude product. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient (B %): 23% B to 53% B in 10 min; Wave Length: 254 nm/220 nm nm; RT1 (min): 7.6) to afford (Z)-5⁵-(4-(1-hydroxycyclopropyl)-1H-pyrazol-1-yl)-2⁵-((2-hydroxyethyl)sulfonyl)-1⁴-methyl-11-oxa-4,6-diaza-5(2,6)-pyridina-1(1,4)-piperidina-2(1,2)-benzenacycloundecaphan-8-en-3-one (6.1 mg, 14% yield).

### Compound 465.

### Step 1: 4-bromo-3-fluoro-1-((2-(trimethylsilyl)ethoxy)methyl)-1H-pyrazole

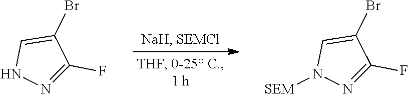 [SMILES: Fc1n[nH]cc1Br ; C[Si](C)(C)CCOCn1cc(Br)c(F)n1]

To a stirred solution of 4-bromo-3-fluoro-1H-pyrazole (10 g, 61 mmol) in THF (100 mL) was added NaH (2.2 g, 91 mmol) portionwise at 0° C. under nitrogen atmosphere. After the resulting mixture was stirred at 0° C. for additional 0.5 hour, SEM-Cl (12 mL) was added. The resulting mixture was stirred at 25° C. for an additional hour then was quenched with water at 0° C. The precipitated solids were collected by filtration and were washed with EA (3×5 mL). The resulting mixture was concentrated under reduced pressure. The residue was purified by silica gel column chromatography, eluted with PE/EA (10:1) to afford 4-bromo-3-fluoro-1-((2-(trimethylsilyl)ethoxy)methyl)-1H-pyrazole (10 g, 45% yield). LCMS (ESI-MS) m/z=295.2[M+H]⁺.

### Step 2: 1-(3-fluoro-1-((2-(trimethylsilyl)ethoxy)methyl)-1H-pyrazol-4-yl)ethan-1-one

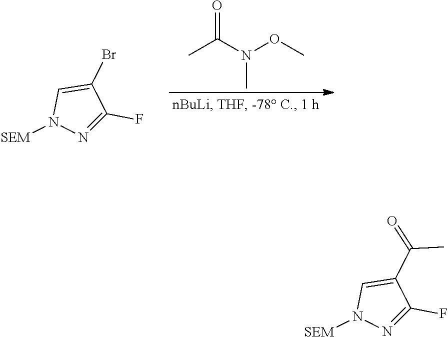

To a stirred solution of 4-bromo-3-fluoro-1-((2-(trimethylsilyl)ethoxy)methyl)-1H-pyrazole (5 g, 20 mmol) in THF (160 mL) was added n-BuLi (1 g, 20 mmol) dropwise at −78° C. under a nitrogen atmosphere. The resulting mixture was stirred at −78° C. for additional 0.5 hour, then N-methoxy-N-methylacetamide (2 g, 20 mmol) was added. The resulting mixture was stirred at −78° C. for additional 1 hour. The reaction was quenched with sat. NH₄Cl (aq.) at 0° C. The precipitated solids were collected by filtration and washed with EA (3×200 mL). The resulting mixture was concentrated under reduced pressure and the resulting residue was purified by silica gel column chromatography, eluted with PE/EA (3:1), to afford 1-(3-fluoro-1-((2-(trimethylsilyl)ethoxy)methyl)-1H-pyrazol-4-yl)ethan-1-one (1.9 g, 40% yield). LCMS (ESI-MS) m/z=259.1 [M+H]⁺.

### Step 3: 4-(1-((tert-butyldimethylsilyl)oxy)vinyl)-3-fluoro-1-((2-(trimethylsilyl)ethoxy)methyl)-1H-pyrazole

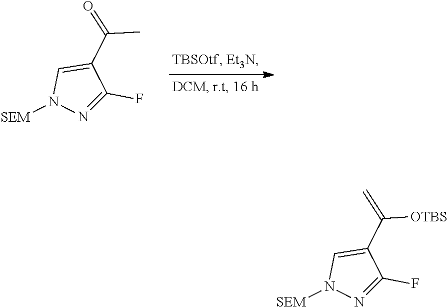 [SMILES: CC(=O)c1cn(COCC[Si](C)(C)C)nc1F ; C=C(O[Si](C)(C)C(C)(C)C)c1cn(COCC[Si](C)(C)C)nc1F]

A solution of 1-(3-fluoro-1-((2-(trimethylsilyl)ethoxy)methyl)-1H-pyrazol-4-yl)ethan-1-one (1.9 g, 7.40 mmol), TEA (3.1 mL) and TBSOTf (5.0 mL) in DCM (20 mL) was stirred at room temperature for 16 hours under a N₂ atmosphere. The reaction was quenched by the addition of water (30 mL) at room temperature. The aqueous layer was extracted with DCM (3×30 mL) and dried over anhydrous Na₂SO₄. After filtration, the crude product was purified by reversed-phase column and eluted with ACN in water (NH₄HCO₃ 0.1%) from 0% to 75% to afford the desired product 4-(1-((tert-butyldimethylsilyl)oxy)vinyl)-3-fluoro-1-((2-(trimethylsilyl)ethoxy)methyl)-1H-pyrazole (1.7 g, 53% yield). LCMS (ESI-MS) m/z=373.1 [M+H]⁺.

### Step 4: 4-(1-((tert-butyldimethylsilyl)oxy)vinyl)-3-fluoro-1-((2-(trimethylsilyl)ethoxy)methyl)-1H-pyrazole

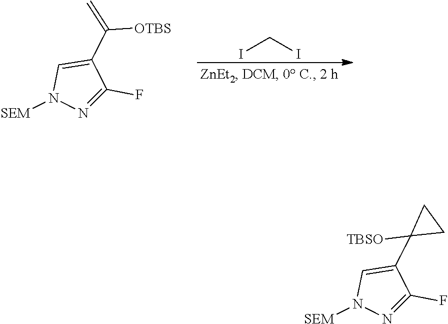

To a stirred solution of 4-(1-((tert-butyldimethylsilyl)oxy)vinyl)-3-fluoro-1-((2-(trimethylsilyl)-ethoxy)methyl)-1H-pyrazole (1.7 g, 6.0 mmol) in DCM (130 mL) was added 1 M ZnEt₂ in DCM (5.8 mL) dropwise at 0° C. under a nitrogen atmosphere. The resulting mixture was stirred at 0° C. for 0.5 h, then diiodomethane (12 g, 46 mmol) was added dropwise over 10 min while stirring at at 0° C. After stirring for an addition 2 h, the reaction mixture was diluted with water (100 mL) and was extracted with DCM (3×80 mL). The combined organic layers were washed with brine (240 mL), dried over anhydrous Na₂SO₄, filtered and concentrated under vacuum. The crude product was purified by reversed-phase column and eluted with ACN in water (NH₄HCO₃ 0.1%) from 0% to 60% to afford to 4-(1-((tert-butyldimethylsilyl)oxy)cyclopropyl)-3-fluoro-1-((2-(trimethylsilyl)ethoxy)methyl)-1H-pyrazole (500 mg, 26% yield). LCMS (ESI-MS) m/z=387.2 [M+H]⁺.

### Step 5: 4-(1-((tert-butyldimethylsilyl)oxy)vinyl)-3-fluoro-1-((2-(trimethylsilyl)ethoxy)methyl)-1H-pyrazole

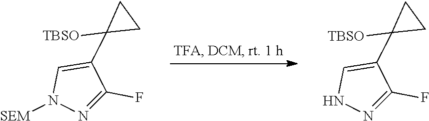 [SMILES: CC(C)(C)[Si](C)(C)OC1(c2cn(COCC[Si](C)(C)C)nc2F)CC1 ; CC(C)(C)[Si](C)(C)OC1(c2c[nH]nc2F)CC1]

TFA (1 mL) was added to a mixture of 4-(1-((tert-butyldimethylsilyl)oxy)cyclopropyl)-3-fluoro-1-((2-(trimethylsilyl)ethoxy)methyl)-1H-pyrazole (500 mg, 1.29 mmol) in DCM (5 mL). After stirring at room temperature for 1 hour, the mixture was diluted with water (5 mL) and was extracted with EA (3×5 mL). The combined organic layers were washed with brine (15 mL), dried over anhydrous Na₂SO₄, filtered, and concentrated under reduced pressure. The crude product was purified by reversed-phase column and eluted with ACN in water (NH₄HCO₃ 0.1%) from 0% to 80%. Fractions with desired mass signal were combined and concentrated under vacuum to afford to 4-(1-((tert-butyldimethylsilyl)oxy)cyclopropyl)-3-fluoro-1H-pyrazole (200 mg, 48% yield). LCMS (ESI-MS) m/z=257.15 [M+H]⁺.

### Step 6: (E)-5⁵-(4-(1-((tert-butyldimethylsilyl)oxy)cyclopropyl)-3-fluoro-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one

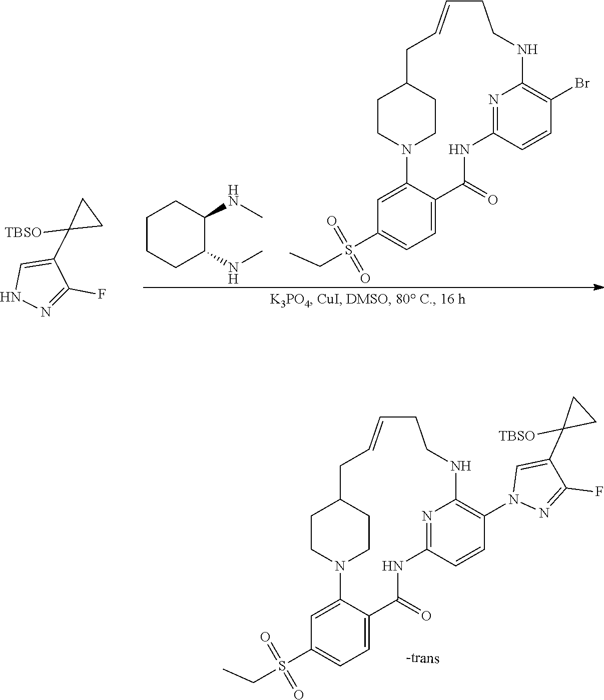

(1R,2R)—N¹,N²-dimethylcyclohexane-1,2-diamine (3.0 mg, 0.02 mmol) and CuI (4.0 mg, 0.02 mmol) were added into a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (100 mg, 0.20 mmol), tert-butyl (1-(1H-pyrazol-4-yl)cyclopropyl)carbamate (251 mg, 1.12 mmol) and K₃PO₄ (119 mg, 0.06 mmol) in DMSO (5 mL) at room temperature under nitrogen atmosphere. The resulting mixture was stirred for 16 hours at 80° C. under nitrogen atmosphere. After cooling to room temperature, the reaction mixture was diluted with H₂O (15 mL) and was extracted with EA (3×15 mL). The combined organic layers were washed with brine (45 mL), dried over anhydrous Na₂SO₄, filtered and concentrated. The crude product was purified by reversed-phase column and eluted with ACN in water (NH₄HCO₃ 0.1%) from 0% to 80% to afford (E)-55-(4-(1-((tert-butyldimethylsilyl)oxy)cyclopropyl)-3-fluoro-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (50 mg, 30% yield). LCMS (ESI-MS) m/z=709.3 [M+H]⁺.

Step 7: Compound 465:1M TBAF in THF (0.1 mL) was added to (E)-5⁵-(4-(1-((tert-butyldimethylsilyl)oxy)cyclopropyl)-3-fluoro-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (50 mg, 0.07 mmol) in THF (2 mL). The mixture was stirred at room temperature for 1 hour. The reaction was poured into EA (10 mL) and extracted with aq. NH₄Cl (3×5 mL). The combined organic layers were washed with brine (15 mL) and dried over anhydrous Na₂SO₄. The crude product was purified by Prep-HPLC with following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 50% B to 80% B in 8 min; Wave Length: 254 nm/220 nm nm; RT1 (min): 7.18) the desired fractions were combined and lyophilized to afford the desired product (E)-2⁵-(ethylsulfonyl)-5⁵-(3-fluoro-4-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one (9.8 mg, 23% yield).

Compound 466 was prepared according to General Procedure 1 using Compound 387 and 1-cyclopropylazetidin-3-ol to afford (E)-N-(55-(3-((1-cyclopropylazetidin-3-yl)oxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.1 mg, 16.2% yield).

Compound 467 was prepared according to General Procedure 1 using Compound 387 and trans-3-(dimethylamino)cyclobutan-1-ol to afford N—((E)-55-(3-((1r,3r)-3-(dimethylamino)cyclobutoxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.1 mg, 5.5% yield).

Compound 468 was prepared according to General Procedure 1 using Compound 388 and 2-(1H-imidazol-1-yl)ethanol to afford (Z)—N-(5⁵-(3-(2-(1H-imidazol-1-yl)ethoxy)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.7 mg, 24% yield).

Compound 469 was prepared according to General Procedure 1 using Compound 388 and trans-3-(dimethylamino)cyclobutan-1-ol to afford N—((Z)-55-(3-((1r,3r)-3-(dimethylamino)cyclobutoxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.5 mg, 7.5% yield).

Compound 470 was prepared according to General Procedure 1 using Compound 387 and 2-(1H-imidazol-1-yl)ethanol to afford (E)-N-(5⁵-(3-(2-(1H-imidazol-1-yl)ethoxy)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.6 mg, 13.6% yield).

Compound 471 was prepared according to General Procedure 1 using Compound 388 and (R)-tetrahydro-2h-pyran-3-ol to afford (R,E)-N-(1⁴-methyl-3-oxo-55-(3-((tetrahydro-2H-pyran-3-yl)oxy)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (1.1 mg, 5.6% yield).

Compound 472 was prepared according to General Procedure 1 using Compound 392 and thiomorpholine 1,1-dioxide hydrochloride to afford (Z)—N-(55-(3-(1,1-dioxidothiomorpholino)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.9 mg, 11.3% yield).

Compound 473 was prepared according to General Procedure 1 using Compound 392 and (S)-3-fluoropiperidine hydrochloride to afford (S,Z)—N-(5⁵-(3-(3-fluoropiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.2 mg, 6.4% yield).

Compound 474 was prepared according to General Procedure 1 using Compound 392 and (S)-3-methoxypyrrolidine to afford (S,Z)—N-(5⁵-(3-(3-methoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.4 mg, 23.3% yield).

Compound 475 was prepared according to General Procedure 1 using Compound 392 and 4,4-difluoropiperidine hydrochloride to afford (Z)—N-(5⁵-(3-(4,4-difluoropiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.7 mg, 8.7% yield).

Compound 476 was prepared according to General Procedure 1 using Compound 392 and (R)-3-methoxypiperidine to afford (R,Z)—N-(5⁵-(3-(3-methoxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (7.6 mg, 40% yield).

Compound 477 was prepared according to General Procedure 1 using Compound 392 and 3,3,4,4-tetrafluoropyrrolidine hydrochloride to afford (Z)—N-(3-oxo-5⁵-(3-(3,3,4,4-tetrafluoropyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2 mg, 10% yield).

Compound 478 was prepared according to General Procedure 1 using Compound 392 and 2-oxazolidone to afford (Z)—N-(3-oxo-55-(3-(2-oxooxazolidin-3-yl)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2 mg, 10.7% yield).

Compound 479 was prepared according to General Procedure 1 using Compound 392 and 4,4-dimethyl-1,4-azasilinane hydrochloride to afford (Z)—N-(5⁵-(3-(4,4-dimethyl-1,4-azasilinan-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.8 mg, 19.6% yield).

Compound 480 was prepared according to General Procedure 1 using Compound 392 and (R)-3-fluoropiperidine hydrochloride to afford (R,Z)—N-(5⁵-(3-(3-fluoropiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3 mg, 16.2% yield).

Compound 481 was prepared according to General Procedure 1 using Compound 392 and 4-fluoropiperidine hydrochloride to afford (Z)—N-(5⁵-(3-(4-fluoropiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.3 mg, 23.1% yield).

Compound 482 was prepared according to General Procedure 1 using Compound 392 and (R)-piperidin-3-ol to afford (R,Z)—N-(5⁵-(3-(3-hydroxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.2 mg, 26.1% yield).

Compound 483 was prepared according to General Procedure 1 using Compound 392 and 4-piperidinol to afford (Z)—N-(55-(3-(4-hydroxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (8.1 mg, 43.2% yield).

Compound 484 was prepared according to General Procedure 1 using Compound 392 and (R)-3-methoxypyrrolidine to afford (R,Z)—N-(5⁵-(3-(3-methoxypyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5 mg, 26.7% yield).

Compound 485 was prepared according to General Procedure 1 using Compound 392 and (R)-pyrrolidine-3-carbonitrile hydrochloride to afford (R,Z)—N-(55-(3-(3-cyanopyrrolidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 11.3% yield).

### Compound 486.

Step 1: 3-(1-(tetrahydro-2H-pyran-2-yl)-1H-pyrazol-4-yl)oxazolidin-2-one

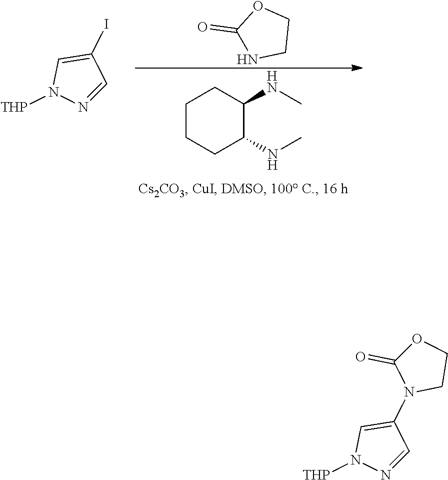

CuI (0.07 g, 0.4 mmol) and (1R,2R)—N¹,N²-dimethylcyclohexane-1,2-diamine (0.05 g, 0.4 mmol) were added to a mixture of 4-iodo-1-(tetrahydro-2H-pyran-2-yl)-1H-pyrazole (1 g, 4 mmol), oxazolidin-2-one (0.6 g, 7 mmol) and Cs₂CO₃ (4 g, 10 mmol) in DMSO (10 mL) at room temperature under nitrogen atmosphere. The resulting mixture was stirred for 16 hours at 100° C. under nitrogen atmosphere. The crude product was purified by reversed-phase column chromatography (220 g) and eluted with MeCN in water (NH₄HCO₃ 0.1%) from 0% to 65%. Fractions with desired mass signal were combined and concentrated under vacuum to afford the desired product 3-(1-(tetrahydro-2H-pyran-2-yl)-1H-pyrazol-4-yl)oxazolidin-2-one (600 mg, 70% yield). LCMS (ESI-MS) m/z=237.9 [M+H]⁺.

### Step 2: 3-(1H-pyrazol-4-yl)oxazolidin-2-one

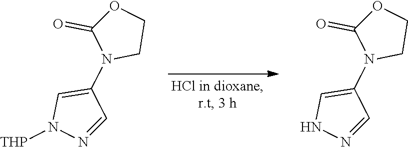

4M HCl in dioxane (6 mL) was added to the stirred mixture of 3-(1-(tetrahydro-2H-pyran-2-yl)-1H-pyrazol-4-yl)oxazolidin-2-one (600 mg, 2.53 mmol), then the resulting mixture was stirred for 3 hours at room temperature. The resulting mixture was concentrated under vacuum to afford crude product 3-(1H-pyrazol-4-yl)oxazolidin-2-one (280 mg, crude). LCMS (ESI-MS) m/z=153.9 [M+H]⁺.

### Step 3: Compound 486

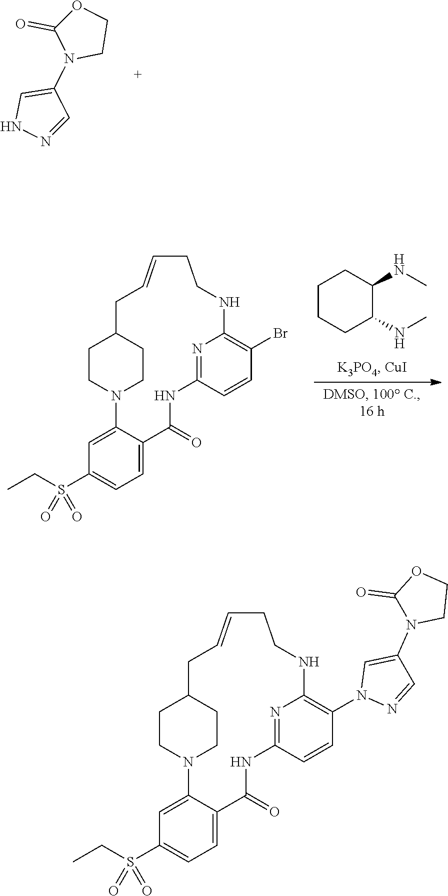

(1R,2R)—N¹,N²-dimethylcyclohexane-1,2-diamine (26.0 mg, 0.18 mmol) and CuI (34.8 mg, 0.18 mmol) were added into a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (975 mg, 1.83 mmol), 3-(1H-pyrazol-4-yl)oxazolidin-2-one (280 mg, 1.83 mmol) and K₃PO₄ (1.16 g, 5.49 mmol) in DMSO (3 mL) at room temperature under a nitrogen atmosphere. After stirring for 16 hours at 100° C., the reaction mixture was cooled, was diluted with water (50 mL), and was extracted with EA (3×50 mL). The combined organic layers were washed with brine (150 mL), dried over anhydrous Na₂SO₄, filtered and concentrated. The crude material purified using flash column chromatography (Silica-CS (40 g) and eluting with EA in PE from 0% to 30% with 50 mL/min flow rate) to afford the crude product. The crude was purified by Prep-HPLC with the following conditions: Column: XBridge Prep Shield RP18 OBD C18 Column, 30*150 mm, 5 μm; Mobile Phase A: Water (10 mmol/L NH₄HCO₃), Mobile Phase B: ACN; Flow rate: 60 mL/min; Gradient (B %): 46% B to 66% B in 10 min; Wave Length: 254 nm/220 nm n; RT1 (min): 8.37 to afford (E)-3-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl)oxazolidin-2-one (16.5 mg, 1.48% yield).

### Compounds 487 and 490.

### Step 1: 1-(1-(tetrahydro-2H-pyran-2-yl)-1H-pyrazol-4-yl)pyrrolidin-2-one

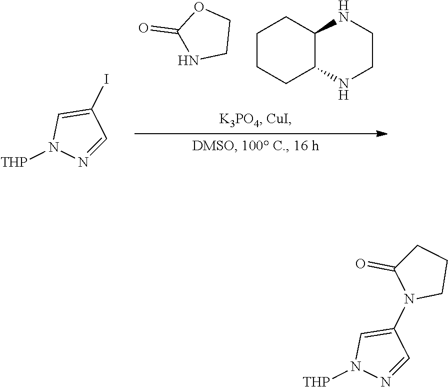

CuI (70 mg, 0.40 mmol) was added to a mixture of 4-iodo-1-(tetrahydro-2H-pyran-2-yl)-1H-pyrazole (1 g, 4 mmol), pyrrolidin-2-one (1 g, 10 mmol), (1R,2R)—N¹,N²-dimethylcyclohexane-1,2-diamine (50.0 mg, 0.4 mmol) and K₃PO₄ (2.3 g, 11 mmol) in DMSO (10 mL) under a nitrogen atmosphere. The mixture was stirred at 100° C. overnight. The reaction was purified by silica gel column chromatography, eluted with PE/EA (1:1) to afford 1-(1-(tetrahydro-2H-pyran-2-yl)-1H-pyrazol-4-yl)pyrrolidin-2-one (1 g, 100%, yield). LCMS (ESI-MS) m/z=236.15 [M+H]⁺.

### Step 2: 1-(1H-pyrazol-4-yl)pyrrolidin-2-one

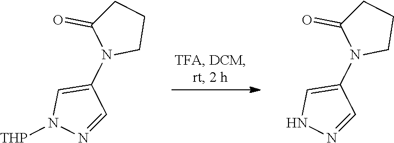

TFA (1 mL, 10.0 mmol) was added to a mixture of 1-(1-(tetrahydro-2H-pyran-2-yl)-1H-pyrazol-4-yl)pyrrolidin-2-one (400 mg, 1.70 mmol) in DCM (5 mL). After stirring at room temperature for 2 hours, the mixture was diluted with water (5 mL) and was extracted with DCM (3×2 mL). The combined organic layers were washed with brine (5×2 mL), dried over anhydrous Na₂SO₄, filtered, and concentrated under reduced pressure to afford the crude 1-(1H-pyrazol-4-yl)pyrrolidin-2-one (200 mg, 66% yield). LCMS (ESI-MS) m/z=152.15 [M+H]⁺.

### Step 3: (E)-2⁵-chloro-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one

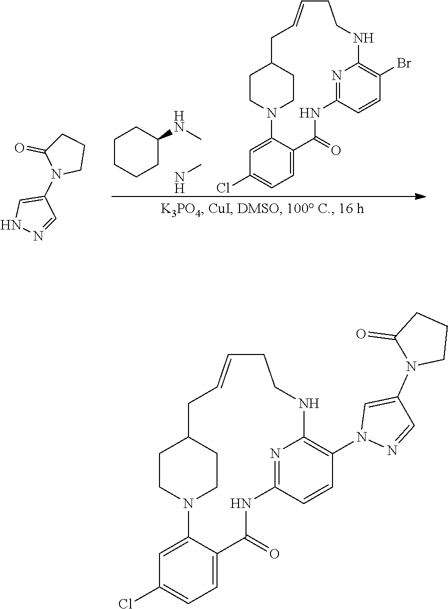

CuI (4.00 mg, 0.02 mmol) was added to a mixture of (E)-5⁵-bromo-2⁵-chloro-4,6-diaza-5(2,6)-pyridina-1(1,4)-piperidina-2(1,2)-benzenacycloundecaphan-9-en-3-one (100 mg, 0.20 mmol), 1-(1H-pyrazol-4-yl)pyrrolidin-2-one (10 mg, 0.07 mmol), (1R,2R)—N¹,N²-dimethylcyclohexane-1,2-diamine (3.0 mg, 0.02 mmol) and K₃PO₄ (134 mg, 0.6 mmol) in DMSO (1.5 mL) under nitrogen atmosphere. The mixture was stirred at 100° C. overnight. The residue was purified by reverse-phase flash chromatography with the following conditions: column, C18 silica gel; mobile phase, aq. 0.04% NH₄CO₃ in ACN, 10% to 90% gradient in 10 min; detector, UV 254 nm to afford (E)-2⁵-chloro-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (60 mg, 47% yield). LCMS (ESI-MS) m/z=546.1 [M+H]⁺.

### Step 4: (E)-2⁵-((2-hydroxyethyl)thio)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one

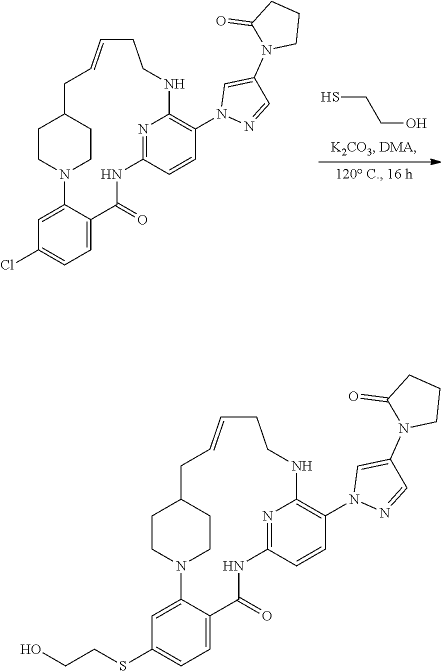

A vial was charged with (E)-2⁵-chloro-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (30 mg, 0.05 mmol), DMA (3 mL), 2-mercaptoethanol (43 mg, 0.55 mmol), and K₂CO₃ (76 mg, 0.55 mmol). The mixture was heated to 120° C. and was stirred for 16 hours. The resulting mixture was cooled to room temperature and was extracted with EA (3×5 mL). The combined organic layers were washed with water (3×3 mL), dried over anhydrous Na₂SO₄, filtered, and concentrated under reduced pressure. The residue was purified by silica gel column chromatography, eluted with PE/EA (1:10) to afford (E)-2⁵-((2-hydroxyethyl)thio)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (30 mg, 79% yield). LCMS (ESI-MS) m/z=588.3 [M+H]⁺.

### Step 5: Compounds 487 and 490

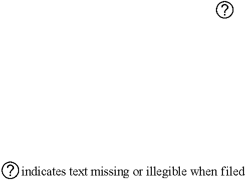

(E)-25-((2-hydroxyethyl)thio)-55-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (30 mg, 0.05 mmol) was added to a stirred mixture of m-CPBA (11 mg, 0.06 mmol) in DCM (1 mL). The resulting mixture was stirred at 25° C. for 1 hour. After cooling to room temperature, the reaction mixture was diluted with H₂O (5 mL) and extracted with EA (2×5 mL). The combined organic layers were washed with brine (10 mL), dried over anhydrous Na₂SO₄, filtered and concentrated. The crude residue was purified by silica gel column chromatography, eluted with PE:EA (1:1) then re-purified by Prep-SFC with the conditions (Column: (R, R)-WHELK-01, 5 μm, 250 mm×20 mm.; Mobile Phase A: Hex (with 0.5% NH3 (2 M in MeOH)), Mobile Phase B: MeOH:DCM=1:1; Flow rate: 20 mL/min; Gradient (B %) 80% B; Wave Length: 254/220 nm; RT1 (min): 7.68; RT2 (min): 19.04; Sample Solvent: EtOH; Injection Volume: 2.0 mL; Number Of Runs: 1; Single injection run time (min): 22) to afford the two products:

First eluting isomer (Compound 487): (E)-2⁵-((2-hydroxyethyl)sulfonyl)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (3.2 mg, 9.6% yield).

Second eluting isomer (Compound 490): (Z)-2⁵-((2-hydroxyethyl)sulfonyl)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (1.4 mg, 4.2% yield).

Compounds 488 and 489 were prepared using (E)-2⁵-((2-hydroxyethyl)thio)-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one from Step 4 in the synthesis of Compound 487 as follows:

To a solution of (E)-2⁵-chloro-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-3-one (20 mg, 0.04 mmol) and 2-hydroxyethane-1-sulfonamide (14 mg, 0.11 mmol) in DMSO (1 mL) were added K₂CO₃ (15 mg, 0.11 mmol) and t-BuXPhos Pd G3 (4.0 mg, 0.004 mmol). After stirring for 16 hours at 80° C. under a nitrogen atmosphere, the resulting mixture was extracted with EA (3×5 mL). The combined organic layers were washed with water (15 mL) and dried over anhydrous Na₂SO₄. After filtration, the filtrate was concentrated under reduced pressure. The crude product (20.0 mg) was purified by Prep-HPLC with the following conditions (Column: (R, R)WhelkO 1; Mobile Phase A: Hex (0.2% FA): (MeOH:DCM=1:1) =30:70; Flow rate: 1 mL/min mL/min; Gradient (B %); Injection Volume: 1 μL mL) to afford the two products:

First eluting isomer (Compound 488) (E)-2-hydroxy-N-(3-oxo-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-25-yl)ethane-1-sulfonamide (1.6 mg, 6.8% yield).

Second eluting isomer (Compound 489) (Z)-2-hydroxy-N-(3-oxo-5⁵-(4-(2-oxopyrrolidin-1-yl)-1H-pyrazol-1-yl)-4,6-diaza-5(2,6)-pyridina-1(1,4)-piperidina-2(1,2)-benzenacycloundecaphan-9-en-2⁵-yl)ethane-1-sulfonamide (1.1 mg, 4.7% yield).

Compound 491 was prepared according to General Procedure 1 using Compound 387 and 1,2,3,4-tetrahydropyrrolo[1,2-a]pyrazine to afford (E)-N-(55-(3-(3,4-dihydropyrrolo[1,2-a]pyrazin-2(1H)-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.3 mg, 28.5% yield).

Compound 492 was prepared according to General Procedure 1 using Compound 387 and 4-methoxypiperidine hydrochloride to afford (E)-N-(5⁵-(3-(4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.1 mg, 14.2% yield).

Compound 493 was prepared according to General Procedure 1 using Compound 387 and 1-methylpiperazine to afford (E)-N-(1⁴-methyl-55-(3-(4-methylpiperazin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (11 mg, 75.1% yield).

Compound 494 was prepared according to General Procedure 1 using Compound 387 and 2-thia-6-azaspiro[3.4]octane 2,2-dioxide hydrochloride to afford (E)-N-(5⁵-(3-(2,2-dioxido-2-thia-6-azaspiro[3.4]octan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (6.9 mg, 42.9% yield).

Compound 495 was prepared according to General Procedure 1 using Compound 387 and 1-(4-hydroxypiperidin-1-yl)ethanone to afford (E)-N-(5⁵-(3-((1-acetylpiperidin-4-yl)oxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.7 mg, 3.9% yield).

Compound 496 was prepared according to General Procedure 1 using Compound 387 and dimethyl-piperidin-4-yl-amine dihydrochloride to afford (E)-N-(5⁵-(3-(4-(dimethylamino)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.5 mg, 9.8% yield).

Compound 497 was prepared according to General Procedure 1 using Compound 387 and (4-fluoropiperidin-4-yl)methanol hydrochloride to afford (E)-N-(5⁵-(3-(4-fluoro-4-(hydroxymethyl)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.7 mg, 17.8% yield).

Compound 498 was prepared according to General Procedure 1 using Compound 387 and (S)-3-(methylsulfonyl)pyrrolidine to afford (E)-N-(5⁵-(3-(4-fluoro-4-(hydroxymethyl)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (10.2 mg, 64.6% yield).

Compound 499 was prepared according to General Procedure 1 using Compound 387 and (R)-3-methoxypyrrolidine to afford (R,E)-N-(55-(3-(3-methoxypyrrolidin-1-yl)-1H-pyrazol-1-yl)-14-methyl-3-oxo-11-oxa-4,6-diaza-5(2,6)-pyridina-1(1,4)-piperidina-2(1,2)-benzenacycloundecaphan-8-en-25-yl)ethanesulfonamide (10.1 mg, 69.3% yield).

Compound 500 was prepared according to General Procedure 1 using Compound 387 and (R)-piperidin-3-ol to afford (R,E)-N-(5⁵-(3-(3-hydroxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.1 mg, 42% yield).

Compound 501 was prepared according to General Procedure 1 using Compound 387 and piperidine-4-carbonitrile hydrochloride to afford (E)-N-(55-(3-(4-cyanopiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.2 mg, 21.7% yield).

Compound 502 was prepared according to General Procedure 1 using Compound 387 and (S)-2-(3-pyrrolidinyl)-2-propanol to afford (S,E)-N-(5⁵-(3-(3-(2-hydroxypropan-2-yl)pyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (10.9 mg, 71.2% yield).

Compound 503 was prepared according to General Procedure 1 using Compound 387 and 3-methylsulfonyl-azetidine hydrochloride to afford (E)-N-(1⁴-methyl-5⁵-(3-(3-(methylsulfonyl)azetidin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (9.4 mg, 61.1% yield).

Compound 504 was prepared according to General Procedure 1 using Compound 387 and 4-piperidinol to afford (E)-N-(5⁵-(3-(4-hydroxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.1 mg, 21.1% yield).

Compound 505 was prepared according to General Procedure 1 using Compound 387 and 2-oxa-5-azabicyclo[2.2.1]heptane to afford (E)-N-(5⁵-(3-(2-oxa-5-azabicyclo[2.2.1]heptan-5-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.8 mg, 46.6% yield).

Compound 506 was prepared according to General Procedure 1 using Compound 387 and 2-oxa-6-azaspiro[3.3]heptane to afford (E)-N-(5⁵-(3-(2-oxa-6-azaspiro[3.3]heptan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (11.3 mg, 77.3% yield).

Compound 507 was prepared according to General Procedure 1 using Compound 387 and (R)-pyrrolidine-3-carbonitrile hydrochloride to afford (R,E)-N-(5⁵-(3-(3-cyanopyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.4 mg, 23.3% yield).

Compound 508 was prepared according to General Procedure 1 using Compound 387 and 2,7-diazaspiro[4.4]nonan-1-one hydrochloride to afford (E)-N-(1⁴-methyl-3-oxo-5⁵-(3-(6-oxo-2,7-diazaspiro[4.4]nonan-2-yl)-1H-pyrazol-1-yl)-11-oxa-4,6-diaza-5(2,6)-pyridina-1(1,4)-piperidina-2(1,2)-benzenacycloundecaphanen-2⁵-yl)ethanesulfonamide (0.8 mg, 5% yield).

Compound 509 was prepared according to General Procedure 1 using Compound 387 and 2-methyl-2,6-diazaspiro[3.4]octane to afford (E)-N-(1⁴-methyl-5⁵-(3-(2-methyl-2,6-diazaspiro[3.4]octan-6-yl)-1H-pyrazol-1-yl)-3-oxo-11-oxa-4,6-diaza-5(2,6)-pyridina-1(1,4)-piperidina-2(1,2)-benzenacycloundecaphanen-2⁵-yl)ethanesulfonamide (4.3 mg, 28.1% yield).

Compound 510 was prepared according to General Procedure 1 using Compound 387 and thiomorpholine 1,1-dioxide hydrochloride to afford (E)-N-(55-(3-(1,1-dioxidothiomorpholino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.2 mg, 33.8% yield).

Compound 511 was prepared according to General Procedure 1 using Compound 387 and 3,3-difluoropiperidine hydrochloride to afford (E)-N-(5⁵-(3-(3,3-difluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2 mg, 13% yield).

Compound 512 was prepared according to General Procedure 1 using Compound 387 and (S)-3-methoxypyrrolidine to afford (S,E)-N-(5⁵-(3-(3-methoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6 mg, 41.2% yield).

Compound 513 was prepared according to General Procedure 1 using Compound 387 and 1-oxa-6-azaspiro[3.4]octane to afford (E)-N-(55-(3-(1-oxa-6-azaspiro[3.4]octan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (3.6 mg, 24.2% yield).

Compound 514 was prepared according to General Procedure 1 using Compound 387 and 2-oxa-7-azaspiro[4.4]nonane to afford (E)-N-(55-(3-(2-oxa-7-azaspiro[4.4]nonan-7-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (7.8 mg, 51.3% yield).

Compound 515 was prepared according to General Procedure 1 using Compound 387 and (3R,4R)-3,4-dimethoxypyrrolidine hydrochloride to afford N—((E)-55-(3-((3R,4R)-3,4-dimethoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.5 mg, 22.7% yield).

Compound 516 was prepared according to General Procedure 1 using Compound 388 and 3,3-difluoropyrrolidine to afford (Z)—N-(55-(3-(3,3-difluoropyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2 mg, 11.9% yield).

Compound 517 was prepared according to General Procedure 1 using Compound 388 and 2-methyl-2,6-diazaspiro[3.4]octane dihydrochloride to afford (Z)—N-(1⁴-methyl-5⁵-(3-(2-methyl-2,6-diazaspiro[3.4]octan-6-yl)-1H-pyrazol-1-yl)-3-oxo-11-oxa-4,6-diaza-5(2,6)-pyridina-1(1,4)-piperidina-2(1,2)-benzenacycloundecaphanen-2⁵-yl)ethanesulfonamide (1.2 mg, 7.1% yield).

Compound 518 was prepared according to General Procedure 1 using Compound 388 and 4-methoxypiperidine hydrochloride to afford (Z)—N-(5⁵-(3-(4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.8 mg, 14.2% yield).

Compound 519 was prepared according to General Procedure 1 using Compound 388 and 1-{2,7-diazaspiro[4.4]nonan-2-yl}ethanone to afford (Z)—N-(55-(3-(7-acetyl-2,7-diazaspiro[4.4]nonan-2-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (9.8 mg, 54.7% yield).

Compound 520 was prepared according to General Procedure 1 using Compound 388 and (R)-3-hydroxypyrrolidine hydrochloride to afford (R,Z)—N-(5⁵-(3-(3-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1 mg, 6.5% yield).

Compound 521 was prepared according to General Procedure 1 using Compound 388 and thiomorpholine-1-oxide hydrochloride to afford (Z)—N-(1⁴-methyl-55-(3-(1-oxidothiomorpholino)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (1.6 mg, 9.4% yield).

Compound 522 was prepared according to General Procedure 1 using Compound 388 and (S)-(+)-3-fluoropyrrolidine hydrochloride to afford (S,Z)—N-(55-(3-(3-fluoropyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.2 mg, 11.4% yield).

Compound 523 was prepared according to General Procedure 1 using Compound 388 and (S)-3-hydroxypyrrolidine hydrochloride to afford (S,Z)—N-(5⁵-(3-(3-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.5 mg, 29.4% yield).

Compound 524 was prepared according to General Procedure 1 using Compound 388 and 2-thia-6-azaspiro[3.4]octane 2,2-dioxide hydrochloride to afford (Z)—N-(5⁵-(3-(2,2-dioxido-2-thia-6-azaspiro[3.4]octan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (11.6 mg, 65.2% yield).

Compound 525 was prepared according to General Procedure 1 using Compound 388 and dimethyl-piperidin-4-yl-amine dihydrochloride to afford (Z)—N-(55-(3-(4-(dimethylamino)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.3 mg, 16.5% yield).

Compound 526 was prepared according to General Procedure 1 using Compound 388 and 4-oxa-7-azaspiro[2.5]octane hydrochloride to afford (Z)—N-(5⁵-(3-(4-oxa-7-azaspiro[2.5]octan-7-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (1.6 mg, 8.4% yield).

Compound 527 was prepared according to General Procedure 1 using Compound 388 and 1-methylpiperazine to afford (Z)—N-(1⁴-methyl-55-(3-(4-methylpiperazin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (5.7 mg, 29.8% yield).

Compound 528 was prepared according to General Procedure 1 using Compound 388 and 2,7-diazaspiro[4.4]nonan-3-one hydrochloride to afford (Z)—N-(1⁴-methyl-3-oxo-5⁵-(3-(8-oxo-2,7-diazaspiro[4.4]nonan-2-yl)-1H-pyrazol-1-yl)-11-oxa-4,6-diaza-5(2,6)-pyridina-1(1,4)-piperidina-2(1,2)-benzenacycloundecaphanen-2⁵-yl)ethanesulfonamide (3.7 mg, 18.3% yield).

Compound 529 was prepared according to General Procedure 1 using Compound 388 and 2-oxazolidone to afford (Z)—N-(1⁴-methyl-3-oxo-55-(3-(2-oxooxazolidin-3-yl)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (6 mg, 37.5% yield).

Compound 530 was prepared according to General Procedure 1 using Compound 388 and (3R,4R)-3,4-dimethoxypyrrolidine hydrochloride to afford N—((Z)-55-(3-((3R,4R)-3,4-dimethoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.2 mg, 26% yield).

Compound 531 was prepared according to General Procedure 1 using Compound 388 and 2,7-diazaspiro[4.4]nonan-1-one hydrochloride to afford (Z)—N-(1⁴-methyl-3-oxo-5⁵-(3-(6-oxo-2,7-diazaspiro[4.4]nonan-2-yl)-1H-pyrazol-1-yl)-11-oxa-4,6-diaza-5(2,6)-pyridina-1(1,4)-piperidina-2(1,2)-benzenacycloundecaphanen-2⁵-yl)ethanesulfonamide (0.3 mg, 2% yield).

Compound 532 was prepared according to General Procedure 1 using Compound 388 and 4-piperidinol to afford (Z)—N-(55-(3-(4-hydroxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (11.3 mg, 58.9% yield).

Compound 533 was prepared according to General Procedure 1 using Compound 388 and 2-oxa-7-azaspiro[4.4]nonane to afford (Z)—N-(5⁵-(3-(2-oxa-7-azaspiro[4.4]nonan-7-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (5.5 mg, 32.7% yield).

Compound 534 was prepared according to General Procedure 1 using Compound 388 and (S)-3-methoxypiperidine to afford (S,Z)—N-(55-(3-(3-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.5 mg, 12.7% yield).

Compound 535 was prepared according to General Procedure 1 using Compound 388 and 2-oxa-5-azabicyclo[2.2.1]heptane to afford (Z)—N-(5⁵-(3-(2-oxa-5-azabicyclo[2.2.1]heptan-5-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.9 mg, 36.1% yield).

Compound 536 was prepared according to General Procedure 1 using Compound 388 and (S)-3-methoxypyrrolidine hydrochloride to afford (S,Z)—N-(5⁵-(3-(3-methoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (12.5 mg, 65.4% yield).

Compound 537 was prepared according to General Procedure 1 using Compound 388 and 4,4-dimethyl-1,4-azasilinane hydrochloride to afford (Z)—N-(5⁵-(3-(4,4-dimethyl-1,4-azasilinan-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.1 mg, 10.7% yield).

Compound 538 was prepared according to General Procedure 1 using Compound 388 and 2-oxa-6-azaspiro[3.3]heptane to afford (Z)—N-(55-(3-(2-oxa-6-azaspiro[3.3]heptan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (8.7 mg, 53.8% yield).

Compound 539 was prepared according to General Procedure 1 using Compound 388 and (S)-2-(3-pyrrolidinyl)-2-propanol to afford (S,Z)—N-(5⁵-(3-(3-(2-hydroxypropan-2-yl)pyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (8.7 mg, 44.1% yield).

Compound 540 was prepared according to General Procedure 1 using Compound 388 and (S)-3-(methylsulfonyl)pyrrolidine to afford (S,Z)—N-(1⁴-methyl-55-(3-(3-(methylsulfonyl)pyrrolidin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (11.8 mg, 58.3% yield).

Compound 541 was prepared according to General Procedure 1 using Compound 388 and (R)-3-methoxypiperidine to afford (R,Z)—N-(5⁵-(3-(3-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.9 mg, 30.4% yield).

Compound 542 was prepared according to General Procedure 1 using Compound 388 and 3-methylsulfonyl-azetidine hydrochloride to afford (Z)—N-(1⁴-methyl-5⁵-(3-(3-(methylsulfonyl)azetidin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4 mg, 20% yield).

Compound 543 was prepared according to General Procedure 1 using Compound 388 and 4,4-difluoropiperidine hydrochloride to afford (Z)—N-(5⁵-(3-(4,4-difluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.5 mg, 18% yield).

Compound 544 was prepared according to General Procedure 1 using Compound 388 and thiomorpholine 1,1-dioxide hydrochloride to afford (Z)—N-(55-(3-(1,1-dioxidothiomorpholino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.7 mg, 3.9% yield).

Compound 545 was prepared according to General Procedure 1 using Compound 388 and 1-(4-piperidinyl)cyclopropanol hydrochloride to afford (Z)—N-(5⁵-(3-(4-(1-hydroxycyclopropyl)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.4 mg, 26.9% yield).

Compound 546 was prepared according to General Procedure 1 using Compound 388 and (4-methoxypiperidin-4-yl)methanol to afford (Z)—N-(55-(3-(4-(hydroxymethyl)-4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (11.8 mg, 58.3% yield).

Compound 547 was prepared according to General Procedure 1 using Compound 388 and (3S,4S)-4-fluoropyrrolidin-3-ol hydrochloride to afford N—((Z)-55-(3-((3S,4S)-3-fluoro-4-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.4 mg, 2.4% yield).

Compound 548 was prepared according to the procedure used to prepare Compound 589 using Intermediate AB and 2-bromoethyl methyl ether to afford (Z)-5⁵-bromo-2⁵-((2-methoxyethyl)sulfonyl)-1⁴-methyl-11-oxa-4,6-diaza-5(2,6)-pyridina-1(1,4)-piperidina-2(1,2)-benzenacycloundecaphan-8-en-3-one (11.9 mg, 30.6% yield).

Compound 549 was prepared according to the procedure used to prepare Compound 589 using Intermediate AB and using chloroacetonitrile to afford (Z)-2-((5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)sulfonyl)acetonitrile (13 mg, 34.5% yield).

Compound 550 was prepared according to the procedure used to prepare Compound 589 using Intermediate AB and 1-(2-bromoethyl)-1H-pyrazole to afford (Z)-2⁵-((2-(1H-pyrazol-1-yl)ethyl)sulfonyl)-5⁵-bromo-1⁴-methyl-11-oxa-4,6-diaza-5(2,6)-pyridina-1(1,4)-piperidina-2(1,2)-benzenacycloundecaphan-8-en-3-one (16.4 mg, 40.1% yield).

Compound 551 was prepared according to the procedure used to prepare Compound 589 using Intermediate AB and 4-bromo-2-methylbutan-2-ol to afford (Z)-5⁵-bromo-2⁵-((3-hydroxy-3-methylbutyl)sulfonyl)-1⁴-methyl-11-oxa-4,6-diaza-5(2,6)-pyridina-1(1,4)-piperidina-2(1,2)-benzenacycloundecaphan-8-en-3-one (8.4 mg, 20.9% yield).

Compound 552 was prepared according to the procedure used to prepare Compound 589 using Intermediate AC and 2-bromoethyl methyl ether to afford (E)-5⁵-bromo-2⁵-((2-methoxyethyl)sulfonyl)-1⁴-methyl-11-oxa-4,6-diaza-5(2,6)-pyridina-1(1,4)-piperidina-2(1,2)-benzenacycloundecaphan-8-en-3-one (11.7 mg, 30.2% yield).

Compound 553 was prepared according to the procedure used to prepare Compound 589 using Intermediate AC and 3-bromo-1-propanol to afford (E)-5⁵-bromo-2⁵-((3-hydroxypropyl)sulfonyl)-1⁴-methyl-11-oxa-4,6-diaza-5(2,6)-pyridina-1(1,4)-piperidina-2(1,2)-benzenacycloundecaphan-8-en-3-one (5.9 mg, 15.1% yield).

Compound 554 was prepared according to the procedure used to prepare Compound 589 using Intermediate AC and using chloroacetonitrile to afford (E)-2-((5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)sulfonyl)acetonitrile (13.8 mg, 36.6% yield).

Compound 555 was prepared according to the procedure used to prepare Compound 589 using Intermediate AC and 1-(2-bromoethyl)-1H-pyrazole to afford (E)-2⁵-((2-(1H-pyrazol-1-yl)ethyl)sulfonyl)-5⁵-bromo-1⁴-methyl-11-oxa-4,6-diaza-5(2,6)-pyridina-1(1,4)-piperidina-2(1,2)-benzenacycloundecaphan-8-en-3-one (2.6 mg, 6.5% yield).

Compound 556 was prepared according to the procedure used to prepare Compound 589 using Intermediate AC and 4-bromo-2-methylbutan-2-ol to afford (E)-5⁵-bromo-2⁵-((3-hydroxy-3-methylbutyl)sulfonyl)-1⁴-methyl-11-oxa-4,6-diaza-5(2,6)-pyridina-1(1,4)-piperidina-2(1,2)-benzenacycloundecaphan-8-en-3-one (4.6 mg, 13.6% yield).

Compound 557 was prepared according to the procedure used to prepare Compound 589 using Intermediate AC and 1-(bromomethyl)cyclopropane-1-carbonitrile to afford (E)-1-(((5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)sulfonyl)methyl)cyclopropane-1-carbonitrile (13.1 mg, 32.8% yield).

Compound 558 was prepared according to the procedure used to prepare Compound 589 using Intermediate AC and 2-bromoethyl methyl sulfone to afford (E)-5⁵-bromo-1⁴-methyl-2⁵-((2-(methylsulfonyl)ethyl)sulfonyl)-11-oxa-4,6-diaza-5(2,6)-pyridina-1(1,4)-piperidina-2(1,2)-benzenacycloundecaphan-8-en-3-one (1.2 mg, 2.8% yield).

Compound 559 was prepared according to the procedure used to prepare Compound 589 using Intermediate AC and 3-bromopropionitrile to afford (E)-3-((5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)sulfonyl)propanenitrile (2.7 mg, 7% yield).

Compound 560 was prepared according to the procedure outlined in Compound 402 using (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide and heating the reaction mixture at 100° C. to afford (E)-N-(1⁴-methyl-5⁵-(4-(oxetan-3-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4.0 mg, 7.4% yield).

### Compounds 561 and 562.

### Step 1: 1-(tetrahydro-2H-pyran-2-yl)-4-((tetrahydrofuran-3-yl) oxy)-1H-pyrazole

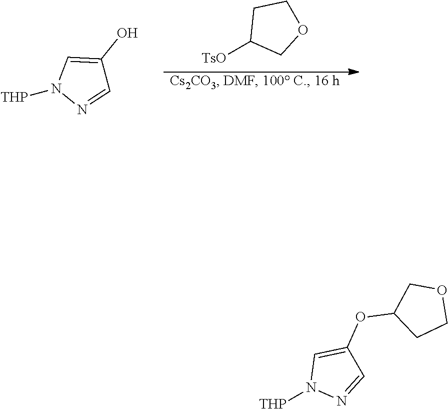

Cs₂CO₃ (3.49 g, 10.7 mmol) was added to a stirred mixture of 1-(tetrahydro-2H-pyran-2-yl)-1H-pyrazol-4-ol (900 mg, 5.35 mmol) and tetrahydrofuran-3-yl 4-methylbenzenesulfonate (1.30 g, 5.35 mmol) in DMF (20 mL). The resulting mixture was stirred at 100° C. overnight. After cooling to room temperature, the reaction mixture was filtered and washed with DMF. The residue was purified by reversed-phase flash chromatography with the following conditions: Column, C18 silica gel; mobile phase, aq. 0.01% NH₄HCO₃ in ACN, 0% to 100% gradient in 20 min; detector, UV 220 nm to afford 1-(tetrahydro-2H-pyran-2-yl)-4-((tetrahydrofuran-3-yl) oxy)-1H-pyrazole (820 mg, 64.3% yield). LCMS (ESI-MS) m/z=239.1 [M+H]⁺.

### Step 2: 4-((tetrahydrofuran-3-yl) oxy)-1H-pyrazole

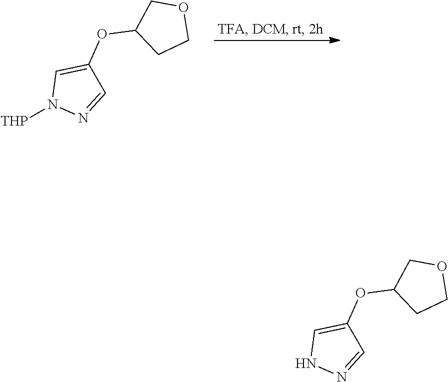

TFA (7 mL) was added to 1-(tetrahydro-2H-pyran-2-yl)-4-((tetrahydrofuran-3-yl) oxy)-1H-pyrazole (800 mg, 3.36 mmol) in DCM (18 mL). The mixture was stirred at room temperature for 2 hours. The mixture was basified to pH 9 with saturated aq. Na₂CO₃ (30 mL) and extracted with EA (3×50 mL). The combined organic layers were washed with brine (100 mL), dried over anhydrous Na₂SO₄, filtered and concentrated under vacuum to afford 4-((tetrahydrofuran-3-yl) oxy)-1H-pyrazole (700 mg crude). LCMS (ESI-MS) m/z=155.1 [M+H]⁺.

### Step 3: (E)-N-(3-oxo-5⁵-(4-((tetrahydrofuran-3-yl) oxy)-1H-pyrazol-1-yl)-4,6-diaza-5(2,6)-pyridina-1(1,4)-piperidina-2(1,2)-benzenacycloundecaphan-9-en-2⁵-yl) ethanesulfonamide

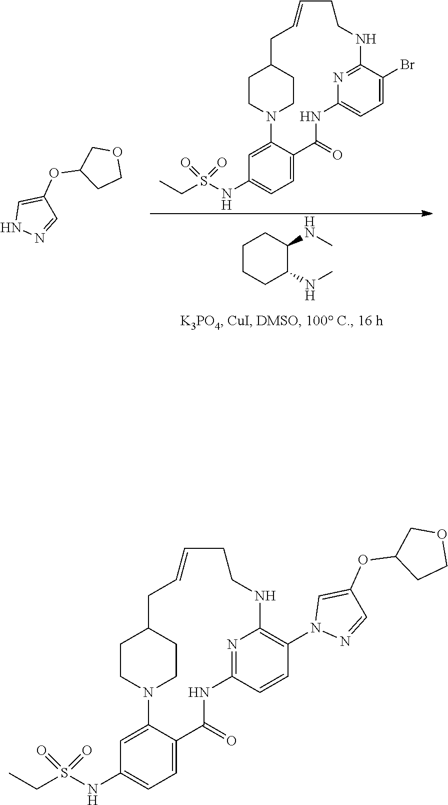

K₃PO₄ (5 mg, 0.21 mmol) was added to the mixture of (E)-N-(5⁵-bromo-3-oxo-4,6-diaza-5(2,6)-pyridina-1(1,4)-piperidina-2(1,2)-benzenacycloundecaphan-9-en-25-yl)ethanesulfonamide (50 mg, 0.09 mmol), 4-((tetrahydrofuran-3-yl)oxy)-1H-pyrazole (100 mg, 0.64 mmol), (1R,2R)—N¹, N²-dimethylcyclohexane-1,2-diamine (2 mg, 0.01 mmol) and CuI (2 mg, 0.01 mmol) in DMSO (5 mL) under nitrogen atmosphere. The mixture was stirred at 100° C. overnight. After cooling to room temperature, the reaction mixture was purified by reversed-phase flash chromatography with the following conditions: Column, C18 silica gel; mobile phase, aq. 0.01% TFA in ACN, 0% to 100% gradient in 10 min; detector, UV 254 nm to afford crude produce. The crude product purified by Prep-HPLC, with the following conditions: Column: X-Bridge Prep OBD C18 Column, 30*150 mm, 5 μm; Mobile Phase A: water (10 mmol/L NH₄HCO₃), Mobile Phase B: ACN; Flow rate: 76 mL/min; Gradient (B %): 46% B to76% B in 10 min; Wave Length: 254 nm/220 nm; RT1 (min): 8.28 to afford (E)-N-(3-oxo-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (30 mg, 53% yield).

Step 4: Compounds 561 and 562: (E)-N-(3-oxo-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (30 mg, 0.048 mmol) was separated by Prep-Chiral HPLC with the following conditions: Column: CHIRALPAK IF3; Mobile Phase A: Hex (0.5% 2M NH₃ MeOH): (MeOH:DCM=1:1)=60:40; Flow rate: 1 mL/min; Gradient (B %); Injection Volume: 1 mL to afford the two products:

First eluting isomer (Compound 561): (S, E)-N-(3-oxo-5⁵-(4-((tetrahydrofuran-3-yl) oxy)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (6.2 mg, 21% yield).

Second eluting isomer (Compound 562): (R, E)-N-(3-oxo-5⁵-(4-((tetrahydrofuran-3-yl) oxy)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (4.9 mg, 16% yield).

### Compounds 563 and 564.

### Step 1: (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one

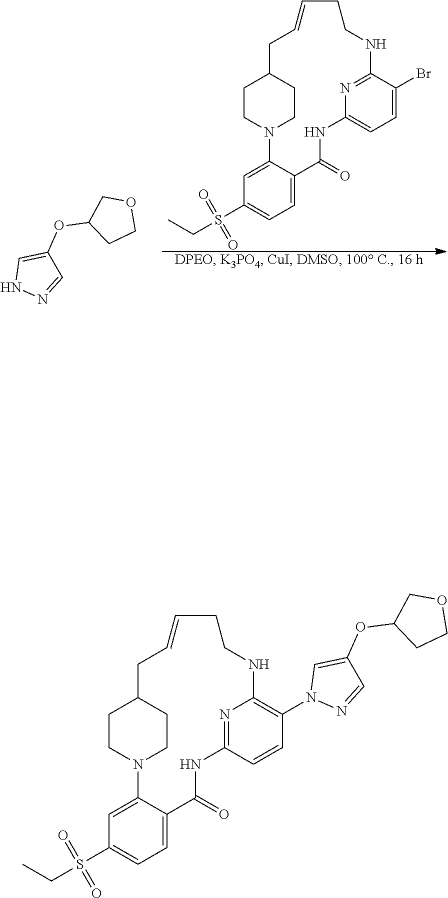

t-BuONa (3.0 equiv) was added to a mixture of ((E)-5⁵-bromo-2⁵-(ethylsulfonyl)-4,6-diaza-5(2,6)-pyridina-1(1,4)-piperidina-2(1,2)-benzenacycloundecaphan-9-en-3-one (1.0 equiv), 4-((tetrahydrofuran-3-yl)oxy)-1H-pyrazole (7.0 equiv), CuI (0.10 equiv) and N¹,N²-diphenethyloxalamide (0.10 equiv) in DMSO (40 V) under nitrogen atmosphere. The mixture was stirred at 100° C. overnight. The reaction mixture was purified by reversed-phase flash chromatography with the following conditions: Column, C18 silica gel; mobile phase, aq. 0.01% TFA in ACN, 0% to 100% gradient in 10 min; detector, UV 220 nm to afford crude product. The crude product was further purified by reversed-phase flash chromatography, with the following conditions: Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min; Gradient (B %): 50% B to 80% B in 8 min; Wave Length: 254 nm/220 nm; RT1 (min): 7.9 to afford (E)-2⁵-(ethylsulfonyl)-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-3-one (53% yield). LCMS (ESI-MS) m/z=607.3 [M+H]⁺.

Step 2: Compounds 563 and 564: (E)-2⁵-(ethylsulfonyl)-5⁵-(4-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-3-one was purified by preparative chiral HPLC with the following conditions: Column: CHIRALPAK 1F3; Mobile Phase A: Hex (0.5% 2M NH3MeOH): (MeOH:DCM=1:1)=60:40; Flow rate: 1 mL/min; Gradient (B %); Injection Volume: 1 mL to afford the two products:

First eluting isomer (Compound 563): enantiopure (E)-2⁵-(ethylsulfonyl)-5⁵-(4-((tetrahydrofuran-3-yl) oxy)-1H-pyrazol-1-yl)-4,6-diaza-5(2,6)-pyridina-1(1,4)-piperidina-2(1,2)-benzenacycloundecaphan-9-en-3-one (21% yield).

Second eluting isomer (Compound 564): enantiopure (E)-2⁵-(ethylsulfonyl)-5⁵-(4-((tetrahydrofuran-3-yl) oxy)-1H-pyrazol-1-yl)-4,6-diaza-5(2,6)-pyridina-1(1,4)-piperidina-2(1,2)-benzenacycloundecaphan-9-en-3-one (20% yield).

Compound 565 was prepared according to General Procedure 1 using Compound 391 and 1-methylpiperidin-4-ol to afford (E)-N-(5⁵-(3-((1-methylpiperidin-4-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (5.4 mg, 17.7% yield).

Compound 566 was prepared according to General Procedure 1 using Compound 391 and 1-(4-hydroxypiperidin-1-yl)ethanone to afford (E)-N-(5⁵-(3-((1-acetylpiperidin-4-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (4.4 mg, 16.2% yield).

Compound 567 was prepared according to General Procedure 1 using Compound 391 and cyclobutanol to afford (E)-N-(55-(3-cyclobutoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (3.4 mg, 11.8% yield).

Compound 568 was prepared according to General Procedure 1 using Compound 391 and 7-oxaspiro[3.5]nonan-2-ol to afford (E)-N-(5⁵-(3-((7-oxaspiro[3.5]nonan-2-yl)oxy)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (3.1 mg, 9.8% yield).

Compound 569 was prepared according to General Procedure 1 using Compound 391 and 3-hydroxytetrahydrofuran to afford (E)-N-(3-oxo-5⁵-(3-((tetrahydrofuran-3-yl)oxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (4.1 mg, 16.5% yield).

Compound 570 was prepared according to General Procedure 1 using Compound 391 and 2-(1H-imidazol-1-yl)ethanol to afford (E)-N-(5⁵-(3-(2-(1H-imidazol-1-yl)ethoxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (6.7 mg, 22.1% yield).

Compound 571 was prepared according to General Procedure 1 using Compound 391 and N-(2-hydroxyethyl)pyrrolidine to afford (E)-N-(3-oxo-5⁵-(3-(2-(pyrrolidin-1-yl)ethoxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (6.0 mg, 19.6% yield).

Compound 572 was prepared according to General Procedure 1 using Compound 391 and 2-(methylsulfonyl)-2-azaspiro[3.3]heptan-6-ol to afford (E)-N-(5⁵-(3-((2-(methylsulfonyl)-2-azaspiro[3.3]heptan-6-yl)oxy)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (4.3 mg, 12.8% yield).

Compound 573 was prepared according to General Procedure 1 using Compound 391 and 1-cyclopropylazetidin-3-ol to afford. (E)-N-(5⁵-(3-((1-cyclopropylazetidin-3-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (8.9 mg, 29.3% yield).

Compound 574 was prepared according to General Procedure 1 using Compound 391 and trans-3-(dimethylamino)cyclobutan-1-ol to afford N—((E)-55-(3-((1r,3r)-3-(dimethylamino)cyclobutoxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (7.5 mg, 24.4% yield).

Compound 575 was prepared according to General Procedure 1 using Compound 392 and 1-cyclopropylazetidin-3-ol to afford (Z)—N-(55-(3-((1-cyclopropylazetidin-3-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.4 mg, 4.5% yield).

Compound 576 was prepared according to General Procedure 1 using Compound 392 and cyclobutanol to afford (Z)—N-(5⁵-(3-cyclobutoxy-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.2 mg, 4.2% yield).

Compound 577 was prepared according to General Procedure 1 using Compound 392 and trans-3-(dimethylamino)cyclobutan-1-ol to afford N—((Z)-55-(3-((1r,3r)-3-(dimethylamino)cyclobutoxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 6.8% yield).

Compound 578 was prepared according to General Procedure 1 using Compound 392 and 2-(1H-imidazol-1-yl)ethanol to afford (Z)—N-(5⁵-(3-(2-(1H-imidazol-1-yl)ethoxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (0.6 mg, 2.1% yield).

Compound 579 was prepared according to General Procedure 1 using Compound 392 and N-(2-hydroxyethyl)pyrrolidine to afford (Z)—N-(3-oxo-5⁵-(3-(2-(pyrrolidin-1-yl)ethoxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2.9 mg, 9.6% yield).

Compound 580 was prepared according to General Procedure 1 using Compound 392 and 1-methylpiperidin-4-ol to afford (Z)—N-(5⁵-(3-((1-methylpiperidin-4-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (1.0 mg, 3.3% yield).

Compound 581 was prepared according to General Procedure 1 using Compound 392 and 1-(4-hydroxypiperidin-1-yl)ethanone to afford (Z)—N-(5⁵-(3-((1-acetylpiperidin-4-yl)oxy)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.0 mg, 6.2% yield).

Compound 582 was prepared according to General Procedure 1 using Compound 392 and 2-methyl-2-azaspiro[3.3]heptan-6-ol to afford (Z)—N-(55-(3-((2-methyl-2-azaspiro[3.3]heptan-6-yl)oxy)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (2.1 mg, 6.9% yield).

Compound 583 was prepared according to General Procedure 1 using Compound 392 and 2-(methylsulfonyl)-2-azaspiro[3.3]heptan-6-ol to afford (Z)—N-(55-(3-((2-(methylsulfonyl)-2-azaspiro[3.3]heptan-6-yl)oxy)-1H-pyrazol-1-yl)-3-oxo-4,6-diaza-5(2,6)-pyridina-1(1,4)-piperidina-2(1,2)-benzenacycloundecaphanen-2⁵-yl)ethanesulfonamide (2 mg, 6% yield).

Compound 584 was prepared according to General Procedure 1 using Compound 387 and potassium 2-cyanoethyltrifluoroborate to afford (E)-N-(55-(3-(2-cyanoethyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.5 mg, 12% yield).

Compound 585 was prepared according to General Procedure 1 using Compound 387 and potassium cyclobutylmethyltrifluoroborate to afford (E)-N-(5⁵-(3-(cyclobutylmethyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.9 mg, 16.7% yield).

### Compound 586.

### Step 1: (E)-5⁵-(4-(1-((tert-butyldimethylsilyl) oxy) cyclopropyl)-1H-pyrazol-1-yl)-2⁵-((2-hydroxyethyl)-sulfonyl)-4,6-diaza-5(2,6)-pyridina-1(1,4)-piperidina-2(1,2)-benzenacycloundecaphan-9-en-3-one

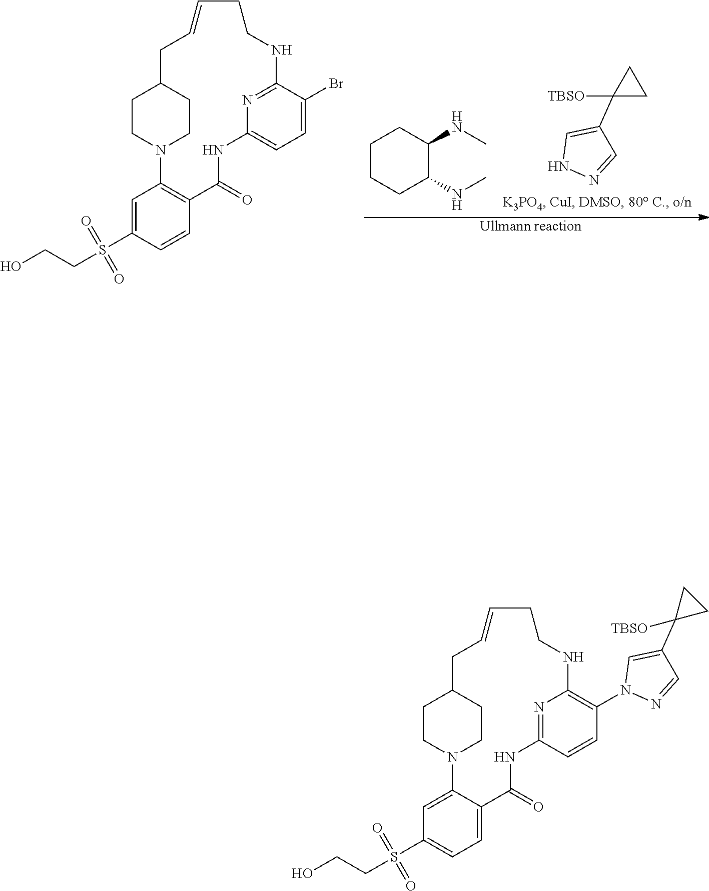

K₃PO₄(3.0 equiv) was added to a mixture of (E)-5⁵-bromo-2⁵-((2-hydroxyethyl)sulfonyl)-4,6-diaza-5(2,6)-pyridina-1(1,4)-piperidina-2(1,2)-benzenacycloundecaphan-9-en-3-one (1.0 equiv), 4-(1-((tert-butyldimethylsilyl)oxy)cyclopropyl)-1H-pyrazole (3.0 equiv), CuI (0.10 equiv) and (1R,2R)—N¹, N²-dimethylcyclohexane-1,2-diamine (0.10 equiv) in DMSO (20 V) under nitrogen atmosphere. The mixture was stirred at 100° C. overnight. The reaction mixture was purified by reversed-phase flash column chromatography with the following conditions: Column, C18 silica gel; mobile phase, aq. 0.04% TFA in ACN, 10% to 82% gradient in 10 min; detector, UV 254 nm to afford (E)-5⁵-(4-(1-((tert-butyldimethylsilyl)oxy)cyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one (31% yield). LCMS (ESI-MS) m/z=707.3 [M+H]⁺.

Step 2: Compound 586: 1M TBAF in THF (1.0 equiv) was added to (E)-5⁵-(4-(1-((tert-butyldimethylsilyl) oxy) cyclopropyl)-1H-pyrazol-1-yl)-2⁵-((2-hydroxyethyl) sulfonyl)-4,6-diaza-5(2,6)-pyridina-1(1,4)-piperidina-2(1,2)-benzenacycloundecaphan-9-en-3-one (1.0 equiv) in THF (40 V). After stirring at room temperature for 2 hours, the reaction mixture was poured into aq. NH₄Cl (30 V) and was extracted with EA (3×30 V). The combined organic layers were washed with aq. NH₄Cl (3×20 V), dried over anhydrous Na₂SO₄, filtered, and concentrated. The crude product was purified by Prep-HPLC with following condition (Column: X-select CSH Prep Fluoro-Phenyl Column, 30*150 mm, 5 μm; Mobile Phase A: Water(0.1% FA), Mobile Phase B: ACN; Flow rate: 60 mL/min mL/min; Gradient (B %): 22% B to 52% B in 10 min; Wave Length: 254 nm/220 nm nm; RT1 (min): 9.5) to afford the product (E)-5⁵-(4-(1-hydroxycyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one (8.7% yield).

### Compounds 587 and 588.

### Step 1: (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one

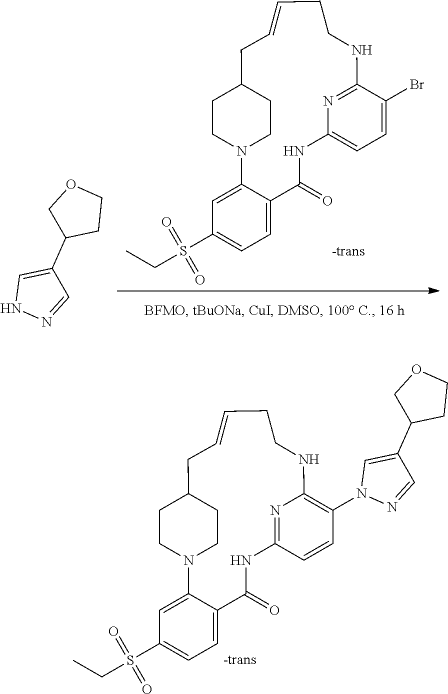

t-BuONa (3.0 equiv) was added to a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (1.0 equiv) 4-(tetrahydrofuran-3-yl)-1H-pyrazole (8.0 equiv), CuI (0.10 equiv) and N¹, N²-bis(furan-2-ylmethyl)oxalamide (0.10 equiv) in DMSO (40 V) under a nitrogen atmosphere. The mixture was stirred at 100° C. overnight. The reaction mixture was purified by reversed-phase flash chromatography with the following conditions: Column, C18 silica gel; mobile phase, aq. 0.1% NH₄HCO₃ in ACN, 10% to 100% gradient in 10 min; detector, UV 220 nm to afford the product (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacy-cloundecaphan-9-en-3-one (36% yield).

Step 2 Compounds 587 and 588: ((E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one was purified by Prep-Chiral HPLC with the following conditions: Column: CHIRALPAK IM, 5 μm, 250 mm×20 mm; Mobile Phase A: Hex, Mobile Phase B: EtOH:DCM=2:7; Flow rate: 20 mL/min; Gradient (B %)₄₅% B; Wave Length: 254/220 n; RT1 (min): 16.52; RT2 (min): 19.13; Sample Solvent: EtOH-HPLC to afford the two products:

First eluting isomer (Compound 587): enantiopure (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one (20% yield).

Second eluting isomer (Compound 588): enantiopure (E)-2⁵-(ethylsulfonyl)-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one (18% yield).

### Compound 589.

### Step 1: (E)-5⁵-bromo-2⁵-(methylsulfonyl)-4,6-diaza-5(2,6)-pyridina-1(1,4)-piperidina-2(1,2)-benzenacycloundecaphan-9-en-3-one

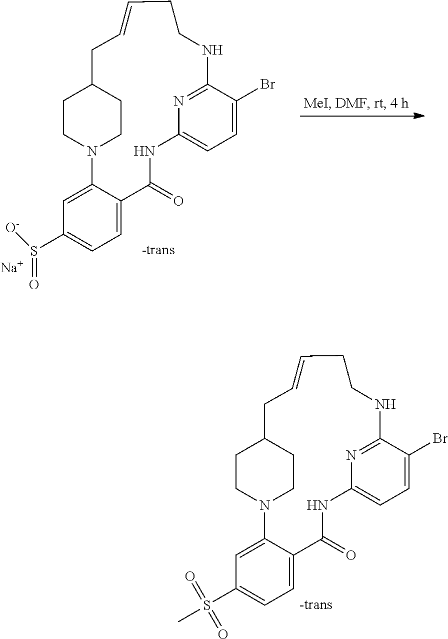 [SMILES: [Na+] ; O=C1Nc2ccc(Br)c(n2)NCC/C=C/CC2CCN(CC2)c2cc(S(=O)[O-])ccc21 ; CS(=O)(=O)c1ccc2c(c1)N1CCC(C/C=C/CCNc3nc(ccc3Br)NC2=O)CC1]

MeI (2.0 equiv) was added to sodium (E)-5⁵-bromo-3-oxo-4,6-diaza-5(2,6)-pyridina-1(1,4)-piperidina-2(1,2)-benzenacycloundecaphan-9-ene-2⁵-sulfinate (Intermediate AA, 1.0 equiv), in DMF (20 V). The mixture was stirred at room temperature for 4 hours. After this time, the reaction mixture was diluted with H₂O (50 V mL) and was extracted with EA (3×50 V). The combined organic layers were washed with brine (150 V), dried over anhydrous Na₂SO₄, filtered, and were concentrated under reduced pressure to afford the crude product. The material was purified by normal phase column chromatography (EA:PE=1:3) to afford (E)-5⁵-bromo-2⁵-(methylsulfonyl)-4,6-diaza-5(2,6)-pyridina-1(1,4)-piperidina-2(1,2)-benzenacycloundecaphan-9-en-3-one (64% yield). LCMS (ESI-MS) m/z=519.5 [M+H]⁺.

### Step 2: (E)-5⁵-(4-(1-((tert-butyldimethylsilyl) oxy) cyclopropyl)-1H-pyrazol-1-yl)-25-(methylsulfonyl)-4,6-diaza-5(2,6)-pyridina-1(1,4)-piperidina-2(1,2)-benzenacycloundecaphan-9-en-3-one

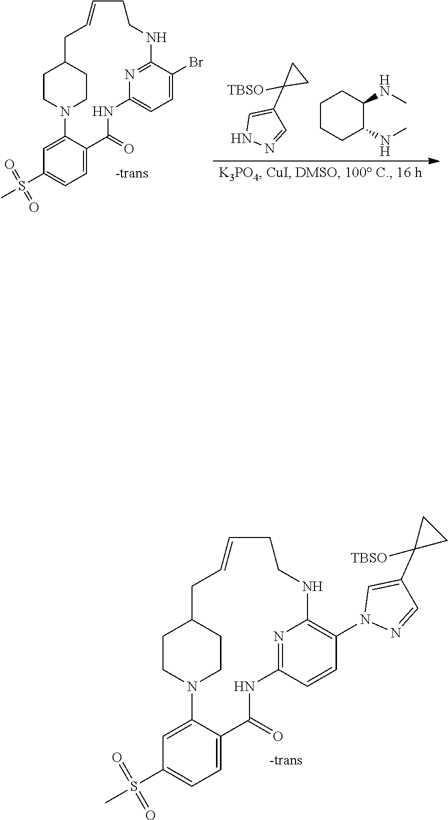

K₃PO₄ (3.0 equiv) was added to a mixture of (E)-5⁵-(4-(1-((tert-butyldimethylsilyl)oxy) cyclopropyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (1.0 equiv), 4-(1-((tert-butyldimethylsilyl)oxy)cyclopropyl)-1H-pyrazole (4. 0 equiv), (1R,2R)—N¹, N²-dimethylcyclohexane-1,2-diamine (0.10 equiv) and CuI (0.10 equiv) in DMSO (40 V) under a nitrogen atmosphere. The mixture was stirred at 100° C. overnight. After cooling to room temperature, the reaction mixture purified by reversed-phase flash chromatography, with the following conditions: Column: X-Bridge Prep OBD C18 Column, 19*250 mm, 5 μm; Mobile Phase A: Water (10 mmol/L NH₄HCO₃), Mobile Phase B: MEOH; Flow rate: 40 mL/min; Gradient (B %): 0% B to 100% B in 10 min; Wave Length: 254 nm/220 nm; RT1 (min): 9.23 to afford (E)-5⁵-(4-(1-((tert-butyldimethylsilyl)oxy)cyclopropyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (31% yield). LCMS (ESI-MS) m/z=677.3 [M+H]⁺.

Step 3: Compound 589: 1M TBAF in THF (2.0 equiv) was added to (E)-5⁵-(4-(1-((tert-butyldimethylsilyl) oxy) cyclopropyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (1.0 equiv) in THF (40 V). The mixture was stirred at room temperature for 2 hours. After this time, the reaction was poured into aq. NH₄Cl (10 mL) at room temperature. The resulting mixture was extracted with EA (3×20 V) and the combined organic layers were washed with aq. NH₄Cl (2×20 V), dried over anhydrous Na₂SO₄, filtered, and concentrated. The crude product was purified by Prep-HPLC with the following conditions: Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 48% B to 78% B in 8 min; Wave Length: 254 nm/220 nm; RT1 (min): 6.23; 7.70 to afford (E)-55-(4-(1-hydroxycyclopropyl)-1H-pyrazol-1-yl)-2⁵-(methylsulfonyl)-4,6-diaza-5(2,6)-pyridina-1(1,4)-piperidina-2(1,2)-benzenacycloundecaphan-9-en-3-one (29% yield).

### Compound 590.

### Step 1: (E)-5⁵-(3-(1-((tert-butyldimethylsilyl)oxy)cyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one

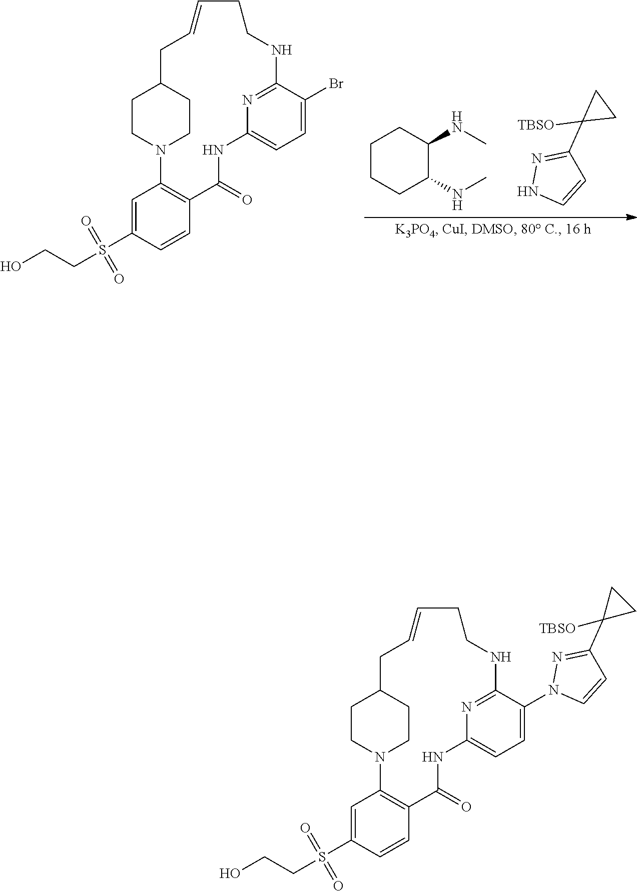

A mixture of (E)-5⁵-bromo-2⁵-((2-hydroxyethyl)sulfonyl)-4,6-diaza-5(2,6)-pyridina-1(1,4)-piperidina-2(1,2)-benzenacycloundecaphan-9-en-3-one (1.0 equiv) in DMSO (15 V) was charged with 3-(1-((tert-butyldimethylsilyl)oxy)cyclopropyl)-1H-pyrazole (4.0 equiv), (1R,2R)—N¹,N²-dimethylcyclohexane-1,2-diamine (0.10 equiv), CuI (0.10 equiv) and K₃PO₄ (5.0 equiv). The resulting solution was heated to 80° C. and stirred for 16 hours. After cooling to room temperature, the reaction mixture was concentrated under reduced pressure and the resulting crude product was purified by reversed-phase column chromatography (elution with ACN in water (NH₄HCO₃ 0.1%) from 0% to 85%) to afford (E)-5⁵-(3-(1-((tert-butyldimethylsilyl)oxy)cyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one (19% yield). LCMS (ESI-MS) m/z=707.3 [M+H]⁺.

Step 2: Compound 589: 1 M TBAF in THF (2.0 equiv) was added to (E)-5⁵-(3-(1-((tert-butyldimethylsilyl)oxy)cyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one (1.0 equiv) in THF (2 mL). After stirring at room temperature for 1 hour, the reaction mixture was poured into aq. NH₄Cl (5 mL) at room temperature. The resulting mixture was extracted with EA (5 mL×3) and the combined organic layers were washed with aq. NH₄Cl (20 mL), dried over anhydrous Na₂SO₄, filtered, and were concentrated under reduced pressure. The residue was purified by Preparative TLC (PE/EA 1:1) to afford (E)-5⁵-(3-(1-hydroxycyclopropyl)-1H-pyrazol-1-yl)-2⁵-((2-hydroxyethyl)sulfonyl)-4,6-diaza-5(2,6)-pyridina-1(1,4)-piperidina-2(1,2)-benzenacycloundecaphan-9-en-3-one (50% yield).

Compound 591 was prepared according to General Procedure 1 using Compound 390 and dimethyl-piperidin-4-yl-amine dihydrochloride to afford (Z)—N-(5⁵-(4-(4-(dimethylamino)piperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.0 mg, 5.7% yield).

Compound 592 was prepared according to General Procedure 1 using Compound 390 and 2-methyl-2,6-diazaspiro[3.4]octane dihydrochloride to afford (Z)—N-(1⁴-methyl-5⁵-(4-(2-methyl-2,6-diazaspiro[3.4]octan-6-yl)-1H-pyrazol-1-yl)-3-oxo-11-oxa-4,6-diaza-5(2,6)-pyridina-1(1,4)-piperidina-2(1,2)-benzenacycloundecaphanen-2⁵-yl)ethanesulfonamide (0.5 mg, 2.6% yield).

Compound 593 was prepared according to General Procedure 1 using Compound 390 and (S)-2-(3-pyrrolidinyl)-2-propanol to afford (S,Z)—N-(5⁵-(4-(3-(2-hydroxypropan-2-yl)pyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (8.3 mg, 48.9% yield).

Compound 594 was prepared according to General Procedure 1 using Compound 390 and 1-methylpiperazine to afford (Z)—N-(1⁴-methyl-5⁵-(4-(4-methylpiperazin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (3.9 mg, 24.2% yield).

Compound 595 was prepared according to General Procedure 1 using Compound 390 and 2-oxa-6-azaspiro[3.3]heptane to afford (Z)—N-(5⁵-(4-(2-oxa-6-azaspiro[3.3]heptan-6-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (9.7 mg, 59.6% yield).

Compound 596 was prepared according to General Procedure 1 using Compound 389 and 1-methyl-4-(methylamino)piperidine to afford (E)-N-(1⁴-methyl-5⁵-(4-(methyl(1-methylpiperidin-4-yl)amino)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (2.7 mg, 16.1% yield).

Compound 597 was prepared according to General Procedure 1 using Compound 389 and 2-methyl-2,6-diazaspiro[3.4]octane to afford (E)-N-(1⁴-methyl-5⁵-(4-(2-methyl-2,6-diazaspiro[3.4]octan-6-yl)-1H-pyrazol-1-yl)-3-oxo-11-oxa-4,6-diaza-5(2,6)-pyridina-1(1,4)-piperidina-2(1,2)-benzenacycloundecaphanen-2⁵-yl)ethanesulfonamide (3.1 mg, 18.2% yield).

Compound 598 was prepared according to General Procedure 1 using Compound 389 and (4-methoxypiperidin-4-yl)methanol to afford (E)-N-(5⁵-(4-(4-(hydroxymethyl)-4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.7 mg, 27.2% yield).

Compound 599 was prepared according to General Procedure 1 using Compound 389 and 2,7-diazaspiro[4.4]nonan-3-one hydrochloride to afford (E)-N-(1⁴-methyl-3-oxo-5⁵-(4-(8-oxo-2,7-diazaspiro[4.4]nonan-2-yl)-1H-pyrazol-1-yl)-11-oxa-4,6-diaza-5(2,6)-pyridina-1(1,4)-piperidina-2(1,2)-benzenacycloundecaphanen-2⁵-yl)ethanesulfonamide (1.4 mg, 6.7% yield).

Compound 600 was prepared according to General Procedure 1 using Compound 389 and 4-fluoropiperidine hydrochloride to afford (E)-N-(5⁵-(4-(4-fluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.4 mg, 17.8% yield).

Compound 601 was prepared according to General Procedure 1 using Compound 389 and thiomorpholine 1,1-dioxide hydrochloride to afford (E)-N-(55-(4-(1,1-dioxidothiomorpholino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.4 mg, 7.1% yield).

Compound 602 was prepared according to General Procedure 1 using Compound 389 and 2-oxa-7-azaspiro[4.4]nonane to afford (E)-N-(5⁵-(4-(2-oxa-7-azaspiro[4.4]nonan-7-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphanen-2⁵-yl)ethanesulfonamide (7.4 mg, 37.5% yield).

Compound 603 was prepared according to General Procedure 1 using Compound 389 and N-methylcyclopropanamine hydrochloride to afford (E)-N-(5⁵-(4-(cyclopropyl(methyl)amino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.9 mg, 12.3% yield).

Compound 604 was prepared according to General Procedure 1 using Compound 389 and 4,4-difluoropiperidine hydrochloride to afford (E)-N-(5⁵-(4-(4,4-difluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.4 mg, 17.4% yield).

Compound 605 was prepared according to General Procedure 1 using Compound 389 and 3-methylsulfonyl-azetidine hydrochloride to afford (E)-N-(1⁴-methyl-5⁵-(4-(3-(methylsulfonyl)azetidin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4.2 mg, 21% yield).

Compound 606 was prepared according to General Procedure 1 using Compound 389 and (R)-piperidin-3-ol to afford (1.2 mg, 6.2% yield). (R,E)-N-(55-(4-(3-hydroxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide.

Compound 607 was prepared according to General Procedure 1 using Compound 389 and 4-methoxypiperidine hydrochloride to afford (E)-N-(5⁵-(4-(4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.2 mg, 11.2% yield).

Compound 608 was prepared according to General Procedure 1 using Compound 389 and (S)-3-hydroxypyrrolidine hydrochloride to afford (S,E)-N-(5⁵-(4-(3-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.3 mg, 17.7% yield).

Compound 609 was prepared according to General Procedure 1 using Compound 389 and (R)-3-fluoropiperidine hydrochloride to afford (R,E)-N-(5⁵-(4-(3-fluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.8 mg, 4.3% yield).

Compound 610 was prepared according to General Procedure 1 using Compound 389 and 1-methylpiperazine to afford (E)-N-(1⁴-methyl-5⁵-(4-(4-methylpiperazin-1-yl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (7 mg, 36.8% yield).

Compound 611 was prepared according to General Procedure 1 using Compound 389 and (R)-3-hydroxypyrrolidine hydrochloride to afford (R,E)-N-(5⁵-(4-(3-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.6 mg, 19.1% yield).

Compound 612 was prepared according to General Procedure 1 using Compound 389 and (R)-3-methoxypiperidine to afford (R,E)-N-(5⁵-(4-(3-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-25-yl)ethanesulfonamide (1.2 mg, 6.1% yield).

Compound 613 was prepared according to General Procedure 1 using Compound 389 and (S)-3-methoxypyrrolidine to afford (S,E)-N-(5⁵-(4-(3-methoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (12.5 mg, 65.4% yield).

Compound 614 was prepared according to General Procedure 1 using Compound 389 and 4-piperidinol to afford (E)-N-(5⁵-(4-(4-hydroxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (14 mg, 73.1% yield).

Compound 615 was prepared according to General Procedure 1 using Compound 389 and (S)-3-methoxypiperidine to afford (S,E)-N-(5⁵-(4-(3-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.5 mg, 33.4% yield).

Compound 616 was prepared according to General Procedure 1 using Compound 389 and 4,4-dimethyl-1,4-azasilinane hydrochloride to afford (E)-N-(5⁵-(4-(4,4-dimethyl-1,4-azasilinan-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (2.6 mg, 13.1% yield).

Compound 617 was prepared according to General Procedure 1 using Compound 389 and thiomorpholine-1-oxide hydrochloride to afford (E)-N-(1⁴-methyl-55-(4-(1-oxidothiomorpholino)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (4.8 mg, 24.5% yield).

### Compound 618.

K₃PO₄ (56 mg, 0.27 mmol) was added to the mixture of (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (50 mg, 0.09 mmol), 4-(1-(methylsulfonyl)cyclopropyl)-1H-pyrazole (66 mg, 0.35 mmol), CuI (17 mg, 0.09 mmol) and (1R,2R)—N¹,N²-dimethylcyclohexane-1,2-diamine (0.51 g, 3.6 mmol) in DMSO (2 mL) under nitrogen atmosphere. The mixture was stirred at 100° C. for 16 hours. After cooling, the reaction mixture was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 69 mL/min mL/min; Gradient (B %): 38% B to 68% B in 10 min; Wave Length: 254 nm/220 nm n; RT1 (min): 8.23) to afford the product (E)-N-(1⁴-methyl-5⁵-(4-(1-(methylsulfonyl)cyclopropyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethane-sulfonamide (11.3 mg, 19% yield).

Compound 619 was prepared according to the procedure outlined in Compound 618 using (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide to afford the product (Z)—N-(1⁴-methyl-5⁵-(4-(1-(methylsulfonyl)cyclopropyl)-1H-pyrazol-1-yl)-3-oxo-11-oxa-4,6-diaza-5(2,6)-pyridina-1(1,4)-piperidina-2(1,2)-benzenacycloundecaphan-8-en-2⁵-yl)ethanesulfonamide (11.8 mg, 20% yield).

### Compound 620.

### Step 1: 1-(1-((2-(trimethylsilyl)ethoxy)methyl)-1H-pyrazol-4-yl)cyclopropan-1-ol

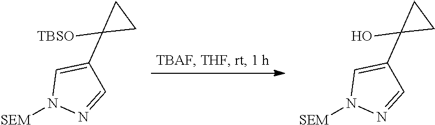 [SMILES: CC(C)(C)[Si](C)(C)OC1(c2cnn(COCC[Si](C)(C)C)c2)CC1 ; "TBAF, THF, rt, 1 h" ; C[Si](C)(C)CCOCn1cc(C2(O)CC2)cn1]

1 M TBAF in THF (7 mL) was added to 4-(1-((tert-butyldimethylsilyl)oxy)cyclopropyl)-1-((2-(trimethylsilyl)ethoxy)methyl)-1H-pyrazole (1 g, 3 mmol) in THF (10 mL). The mixture was stirred at room temperature for 2 hours. The reaction was poured into EA (40 mL) and extracted with aq. NH₄Cl (3×50 mL). The combined organic layers were washed with brine (100 mL), dried over anhydrous Na₂SO₄, filtered, and concentrated under reduced pressure to afford crude product 1-(1-((2-(trimethylsilyl)ethoxy)methyl)-1H-pyrazol-4-yl)cyclopropan-1-ol (520 mg, 60% yield). LCMS (ESI-MS) m/z=255.1 [M+H]⁺.

### Step 2: 4-(1-methoxycyclopropyl)-1-((2-(trimethylsilyl)ethoxy)methyl)-1H-pyrazole

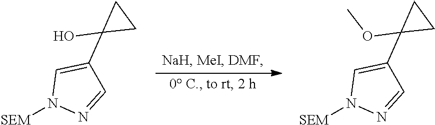 [SMILES: C[Si](C)(C)CCOCn1cc(C2(O)CC2)cn1 ; "0° C., to rt, 2 h" ; "NaH, MeI, DMF," ; COC1(c2cnn(COCC[Si](C)(C)C)c2)CC1]

NaH (124 mg, 60% in mineral oil, 3.07 mmol) was added to 1-(1-((2-(trimethylsilyl)ethoxy)methyl)-1H-pyrazol-4-yl)cyclopropan-1-ol (520 mg, 2.04 mmol) in DMF (10 mL) at 0° C. The mixture was stirred at 0° C. for 1 hour. Then MeI (580 mg, 4.09 mmol) was added to the reaction mixture and the resulting mixture was stirred at room temperature for 1 hour. After this time, mixture was diluted with aq. NH₄Cl (50 mL) and was extracted with EA (3×50 mL). The combined organic layers were washed with brine (150 mL), dried over anhydrous Na₂SO, filtered and concentrated under reduced pressure to afford the crude product 4-(1-methoxycyclopropyl)-1-((2-(trimethylsilyl)ethoxy)methyl)-1H-pyrazole (480 mg, 70% yield). LCMS (ESI-MS) m/z=269.1 [M+H]⁺.

### Step 3: 4-(1-methoxycyclopropyl)-1H-pyrazole

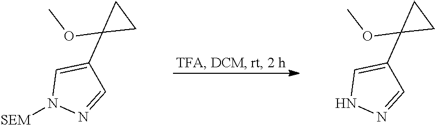

TFA (3 mL) was added to 4-(1-methoxycyclopropyl)-1-((2-(trimethylsilyl)ethoxy)methyl)-1H-pyrazole (480 mg, 1.79 mmol) in DCM (10 mL). The mixture was stirred at room temperature for 1 hour then concentrated under reduced pressure. The residue was dissolved in aq. NaHCO₃ (10 mL) and the resulting mixture was extracted with EA (3×10 mL). The combined organic layers were washed with brine (30 mL), dried over anhydrous Na₂SO₄, filtered, and concentrated under reduced pressure to afford the crude product. The crude product was purified by reversed-phase flash chromatography with the following conditions: column, C18 silica gel; mobile phase, aq. 0.04% TFA in ACN, 10% to 68% gradient in 10 min; detector, UV 254 nm to afford 4-(1-methoxycyclopropyl)-1H-pyrazole (240 mg, 78% yield). LCMS (ESI-MS) m/z=637.2 [M+H]⁺.

### Step 4: Compound 620

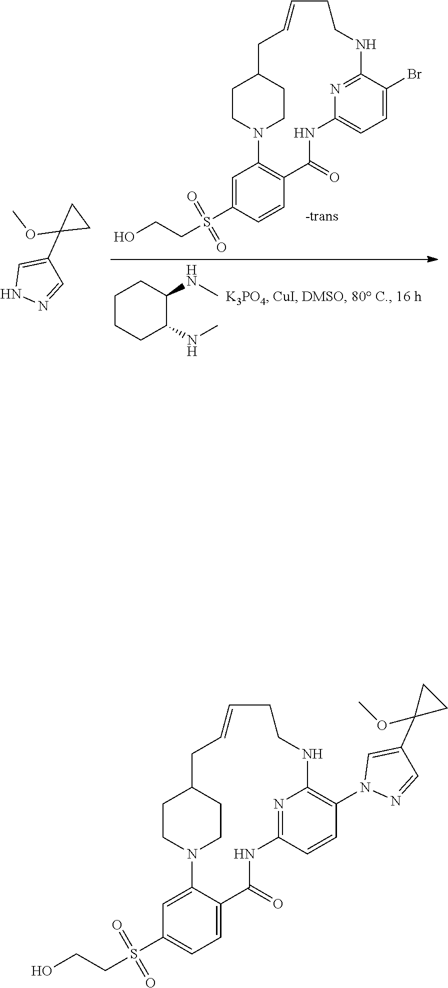

K₃PO₄ (232 mg, 1.09 mmol) was added to the mixture of 4-(1-methoxycyclopropyl)-1H-pyrazole (200 mg, 1.45 mmol), (E)-5⁵-bromo-2⁵-((2-hydroxyethyl)sulfonyl)-4,6-diaza-5(2,6)-pyridina-1(1,4)-piperidina-2(1,2)-benzenacycloundecaphan-9-en-3-one (200 mg, 0.363 mmol), CuI (7 mg, 0.03 mmol) and (1R,2R)—N¹,N²-dimethylcyclohexane-1,2-diamine (6 mg, 0.03 mmol) in DMSO (10 mL) under a nitrogen atmosphere. The mixture was stirred at 100° C. for 16 hours. After cooling, the reaction mixture was purified by reversed-phase flash chromatography with the following conditions: column, C18 silica gel; mobile phase, aq. 0.04% NH₄HCO₃ in ACN, 10% to 89% gradient in 10 min; detector, UV 254 nm to afford the product (E)-2⁵-((2-hydroxyethyl)sulfonyl)-5⁵-(4-(1-methoxycyclopropyl)-1H-pyrazol-1-yl)-4,6-diaza-5(2,6)-pyridina-1(1,4)-piperidina-2(1,2)-benzenacycloundecaphan-9-en-3-one (4.1 mg, 1.7% yield).

### Compound 621.

### Step 1: (E)-5⁵-bromo-2⁵-((2-methoxyethyl)sulfonyl)-4,6-diaza-5(2,6)-pyridina-1(1,4)-piperidina-2(1,2)-benzenacycloundecaphan-9-en-3-one

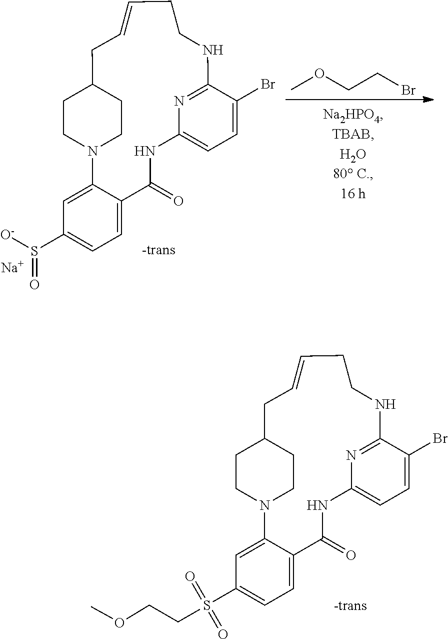

1-bromo-2-methoxyethane (1.0 equiv was added to a mixture of sodium (E)-5⁵-bromo-3-oxo-4,6-diaza-5(2,6)-pyridina-1(1,4)-piperidina-2(1,2)-benzenacycloundecaphan-9-ene-2⁵-sulfinate (Intermediate AA, 1.0 equiv), Na₂HPO₄ (1.0 equiv) and TBAB (1.0 equiv) in H₂O (20 V) at room temperature. The resulting mixture was stirred for 16 hours at 80° C. After cooling, the reaction mixture was diluted with water (20 V) and extracted with EA (3×20 V). The combined organic layers were washed with brine (50 V), dried over anhydrous Na₂SO₄, filtered, and the resulting mixture was concentrated under reduced pressure to afford the crude product (E)-5⁵-bromo-2⁵-((2-methoxyethyl)sulfonyl)-4,6-diaza-5(2,6)-pyridina-1(1,4)-piperidina-2(1,2)-benzenacycloundecaphan-9-en-3-one. LCMS (ESI-MS) m/z=563.5 [M+H]⁺.

### Step 2: (E)-5⁵-(4-(1-((tert-butyldimethylsilyl)oxy)cyclopropyl)-1H-pyrazol-1-yl)-2⁵-((2-methoxyethyl)sulfonyl)-4,6-diaza-5(2,6)-pyridina-1(1,4)-piperidina-2(1,2)-benzenacycloundecaphan-9-en-3-one

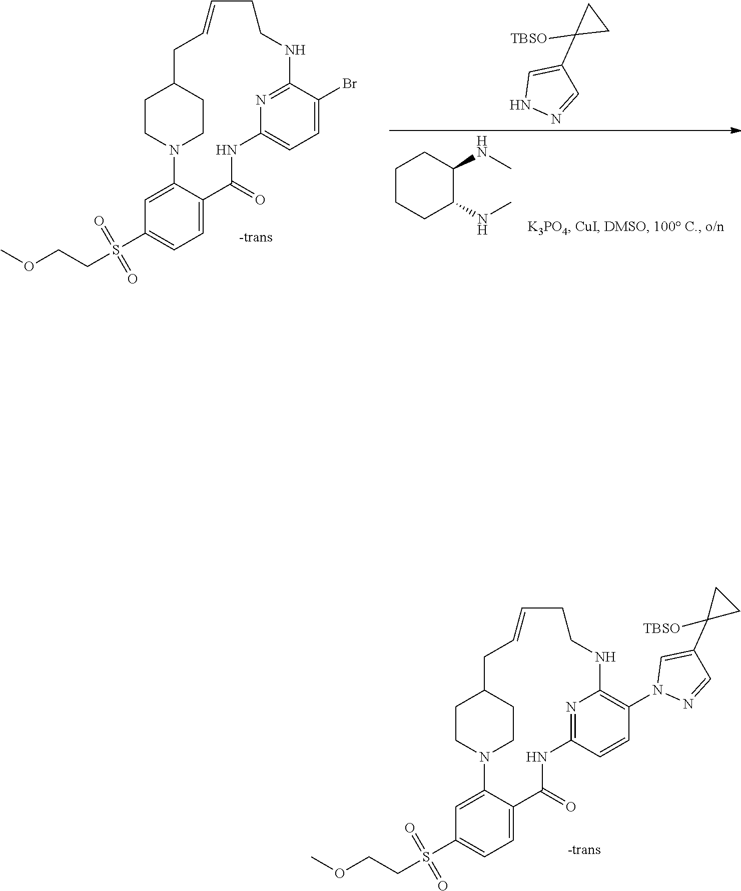

(1R,2R)—N¹,N²-dimethylcyclohexane-1,2-diamine (0.10 equiv) and cuprous iodide (0.10 equiv) were added to a mixture of (E)-5⁵-bromo-2⁵-((2-methoxyethyl)sulfonyl)-4,6-diaza-5(2,6)-pyridina-1(1,4)-piperidina-2(1,2)-benzenacycloundecaphan-9-en-3-one (1.0 equiv), 4-(1-((tert-butyldimethylsilyl)oxy)cyclopropyl)-1H-pyrazole (2.0 equiv) and K₃PO₄ (3.0 equiv) in DMSO (30 V) at room temperature under nitrogen atmosphere. The resulting mixture was stirred for 16 hours at 100° C., then the reaction mixture was cooled, was diluted with water (40 V), and was extracted with EA (3×20 V). The combined organic layers were washed with brine (40 V), dried over anhydrous Na₂SO₄, filtered, and concentrated under reduced pressure. The resulting material was purified by normal phase chromatography ([PE:EA][1:3]) to afford the product (E)-5⁵-(4-(1-((tert-butyldimethylsilyl)oxy)cyclopropyl)-1H-pyrazol-1-yl)-2⁵-((2-methoxyethyl)sulfonyl)-4,6-diaza-5(2,6)-pyridina-1(1,4)-piperidina-2(1,2)-benzenacycloundecaphan-9-en-3-one (17% yield). LCMS (ESI-MS) m/z=721.0 [M+H]⁺.

Step 3: Compound 621: 1M TBAF in THF (2.0 equiv) was added to a mixture of (E)-5⁵-(4-(1-((tert-butyldimethylsilyl)oxy)cyclopropyl)-1H-pyrazol-1-yl)-2⁵-((2-methoxyethyl)sulfonyl)-4,6-diaza-5(2,6)-pyridina-1(1,4)-piperidina-2(1,2)-benzenacycloundecaphan-9-en-3-one (1.0 equiv) in THF (20 V), then the resulting mixture was stirred for 1 hour at room temperature. The reaction mixture was diluted with aq. NH₄Cl (10 V) and extracted with EA (3×20 V). The combined organic layers were washed with brine (40 V), dried over anhydrous Na₂SO₄, filtered, and concentrated under reduced pressure to afford the crude product. The crude was purified by preparative HPLC to afford (E)-5⁵-(4-(1-hydroxycyclopropyl)-1H-pyrazol-1-yl)-2⁵-((2-methoxyethyl)sulfonyl)-4,6-diaza-5(2,6)-pyridina-1(1,4)-piperidina-2(1,2)-benzenacycloundecaphan-9-en-3-one (15% yield).

### Compound 622.

K₃PO₄ (3.0 equiv) was added to the mixture of (E)-5⁵-bromo-2⁵-(methylsulfonyl)-4,6-diaza-5(2,6)-pyridina-1(1,4)-piperidina-2(1,2)-benzenacycloundecaphan-9-en-3-one (1.0 equiv), 4-(1H-pyrazol-4-yl)morpholin-3-one (4.0 equiv), (1R,2R)—N¹, N²-dimethylcyclohexane-1,2-diamine (0.10 equiv) and CuI (0.10 equiv) in DMSO (50 V) under a nitrogen atmosphere. The mixture was stirred at 100° C. overnight. After cooling to room temperature, the resulting mixture was filtered, the filter cake was washed with DCM (3×30 V). The filtrate was concentrated under reduced pressure. The residue was purified by reversed-phase flash column chromatography to afford (E)-4-(1-(2⁵-(methylsulfonyl)-3-oxo-4,6-diaza-5(2,6)-pyridina-1(1,4)-piperidina-2(1,2)-benzenacycloundecaphan-9-en-5⁵-yl)-1H-pyrazol-4-yl)morpholin-3-one (8% yield).

### Compound 623.

K₃PO₄ (3.0 equiv) was added to the mixture of (E)-5⁵-bromo-2⁵-((2-hydroxyethyl)sulfonyl)-4,6-diaza-5(2,6)-pyridina-1(1,4)-piperidina-2(1,2)-benzenacycloundecaphan-9-en-3-one (1.0 equiv) mmol), 4-(1H-pyrazol-4-yl)morpholin-3-one (1.2 equiv), (1R,2R)—N¹, N²-dimethylcyclohexane-1,2-diamine (0.02 equiv) and CuI (0.02 equiv) in DMSO (50 V) under nitrogen atmosphere. The mixture was stirred at 100° C. overnight. After cooling to room temperature, the residue was purified by reversed-phase flash column chromatography to afford (E)-4-(1-(2⁵-((2-hydroxyethyl)sulfonyl)-3-oxo-4,6-diaza-5(2,6)-pyridina-1(1,4)-piperidina-2(1,2)-benzenacycloundecaphan-9-en-55-yl)-1H-pyrazol-4-yl)morpholin-3-one (8% yield).

Compound 624 was prepared according to the procedure outlined in Compound 618 using Compound 304 as the electrophile to afford (E)-N-(55-(4-(1-(methylsulfonyl)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide.

### Compounds 625 and 629.

### Step 1: 4-(oxetan-3-yl)-1-(tetrahydro-2H-pyran-2-yl)-1H-pyrazole

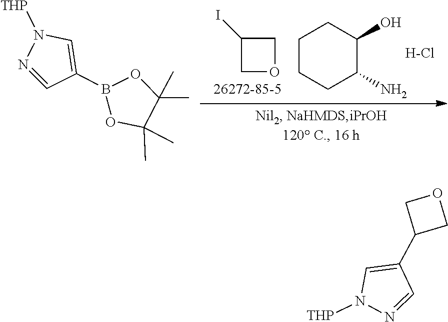

2 M NaHMDS in THF (1.0 equiv) and (1R,2R)-2-aminocyclohexan-1-ol hydrochloride (0.1 equiv) were added to a mixture of 3-iodooxetane (1.2 equiv), 1-(tetrahydro-2H-pyran-2-yl)-4-(4,4,5,5-tetramethyl-1,3,2-dioxaborolan-2-yl)-1H-pyrazole (1.0 equiv) and NiI₂ (0.10 equiv) in iPrOH (10 V) at room temperature under nitrogen atmosphere. The resulting mixture was stirred for 16 hours at 120° C. After cooling to room temperature, the reaction mixture was diluted with water (30 V) and extracted with EA (3×30 V). The combined organic layers were washed with brine (90 V), dried over anhydrous Na₂SO₄, filtered, and concentrated under reduced pressure. The resulting crude product was purified via flash column chromatography Silica-CS and eluting with THF in PE from 0% to 40% with 40 mL/min flow rate. Fractions with desired mass signal were combined and concentrated under vacuum to afford 4-(oxetan-3-yl)-1-(tetrahydro-2H-pyran-2-yl)-1H-pyrazole (60% yield). LCMS (ESI-MS) m/z=209.1 [M+H]⁺.

### Step 2: 4-(oxetan-3-yl)-1H-pyrazole

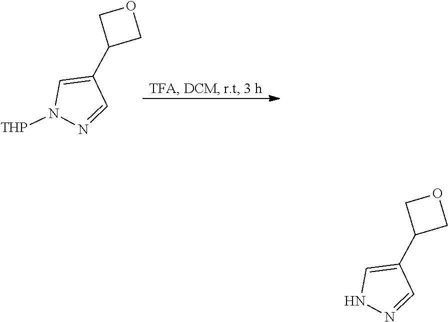

TFA (1.0 equiv) was added to the stirred mixture of 4-(oxetan-3-yl)-1-(tetrahydro-2H-pyran-2-yl)-1H-pyrazole (1.0 equiv) in DCM (15 V), then the resulting mixture was stirred for 3 hours at room temperature. The pH value of the resulting solution was adjusted to 8-9 with 1 N Na₂CO₃. The aqueous layer was extracted with DCM (15 V). The resulting mixture was concentrated under reduced pressure to afford 4-(oxetan-3-yl)-1H-pyrazole (crude). LCMS (ESI-MS) m/z=124.1 [M+H]⁺.

### Step 3: 2⁵-chloro-5⁵-(4-(oxetan-3-yl)-1H-pyrazol-1-yl)-4,6-diaza-5(2,6)-pyridina-1(1,4)-piperidina-2(1,2)-benzenacycloundecaphan-9-en-3-one

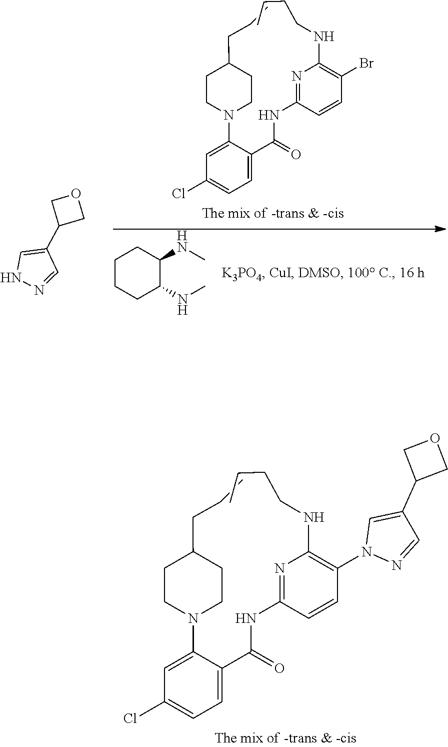

(1R,2R)—N¹,N²-dimethylcyclohexane-1,2-diamine (1.0 equiv) and cuprous iodide (1.0 equiv) were added to a mixture of 5⁵-bromo-2⁵-chloro-4,6-diaza-5(2,6)-pyridina-1(1,4)-piperidina-2(1,2)-benzenacycloundecaphan-9-en-3-one (1.0 equiv), 4-(oxetan-3-yl)-1H-pyrazole (3.0 equiv) and K₃PO₄ (3.0 equiv) in DMSO (3 equiv) at room temperature under a nitrogen atmosphere. After stirring for 16 hours at 100° C. under nitrogen atmosphere, the reaction mixture was diluted with water (10 V) and was extracted with EA (3×10 V). The combined organic layers were washed with brine (30 V), dried over anhydrous Na₂SO₄, filtered, and concentrated under reduced pressure, and purified via flash column chromatography (Silica-CS and eluting with EA in PE from 0% to 75% with 30 mL/min flow rate). Fractions with desired mass signal were combined and concentrated under vacuum to afford 2⁵-chloro-5⁵-(4-(oxetan-3-yl)-1H-pyrazol-1-yl)-4,6-diaza-5(2,6)-pyridina-1(1,4)-piperidina-2(1,2)-benzenacycloundecaphan-9-en-3-one (55% yield). LCMS (ESI-MS) m/z=519.0 [M+H]⁺.

### Step 4: N-(5⁵-(4-(oxetan-3-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide

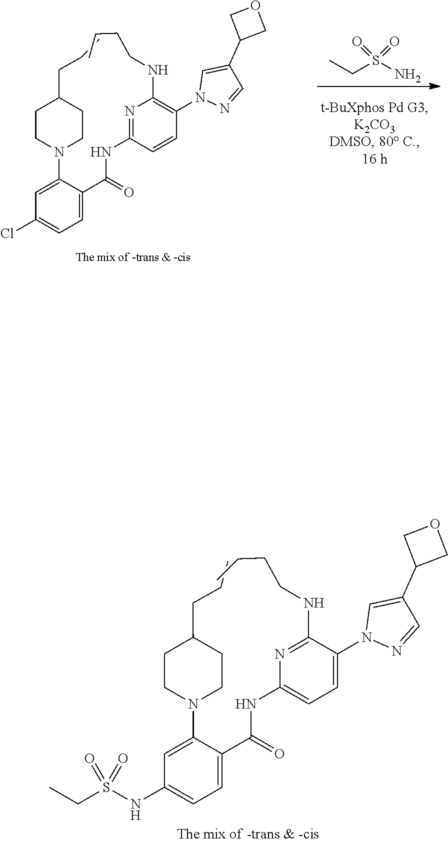

K₂CO₃ (3.0 equiv) was added to a mixture of 2⁵-chloro-5⁵-(4-(oxetan-3-yl)-1H-pyrazol-1-yl)-4,6-diaza-5(2,6)-pyridina-1(1,4)-piperidina-2(1,2)-benzenacycloundecaphan-9-en-3-one (1.0 equiv), ethanesulfonamide (2.0 equiv) and t-BuXphos Pd G3 (1.0 equiv) in DMSO (10 V) at room temperature under a nitrogen atmosphere. After stirring for 16 hours at 80° C., the reaction mixture was diluted with water (20 V) and was extracted with EA (3×20 V). The combined organic layers were washed with brine (40 V), dried over anhydrous Na₂SO₄, filtered, concentrated under reduced pressure, and purified via flash column chromatography (Silica-CS and eluting with EA in PE from 0% to 40% with 30 mL/min flow rate) to afford N-(5⁵-(4-(oxetan-3-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (36% yield). LCMS (ESI-MS) m/z=592.7 [M+H]⁺.

Step 5: Compounds 625 and 629: N-(5⁵-(4-(oxetan-3-yl)-1H-pyrazol-1-yl)-3-oxo-4,6-diaza-5(2,6)-pyridina-1(1,4)-piperidina-2(1,2)-benzena-cycloundecaphan-9-en-2⁵-yl)ethanesulfonamide was separated by preparative chiral HPLC with the following condition: Column: (R, R)-WHELK-01, 5 μm, 250 mm×21.2 mm; Mobile Phase A: Hex, Mobile Phase B: MeOH:DCM=1:1; Flow rate: 20 mL/min; Gradient (B %) 90% B; Wave Length: 254/220 nm; RT1 (min): 4.68; RT2 (min): 8.21; Sample Solvent: EtOH--HPLC; to afford the two products:

First eluting isomer (Compound 625): (E)-N-(5⁵-(4-(oxetan-3-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (32% yield).

Second eluting isomer (Compound 629): (Z)—N-(5⁵-(4-(oxetan-3-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (26% yield).

### Compounds 626 and 627.

### Step 1: 1-(tetrahydro-2H-pyran-2-yl)-4-(tetrahydrofuran-3-yl)-1H-pyrazole

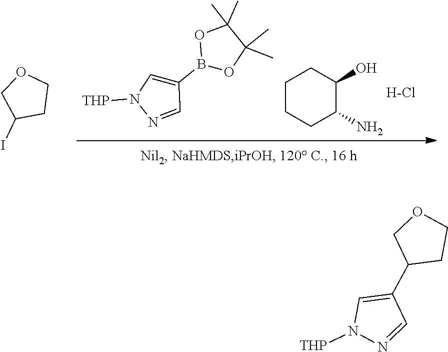

2.5 M NaHMDS in THF (10 mL) was added to the mixture of 3-iodotetrahydrofuran (1 g, 5 mmol), 1-(tetrahydro-2H-pyran-2-yl)-4-(4,4,5,5-tetramethyl-1,3,2-dioxaborolan-2-yl)-1H-pyrazole (2 g, 8 mmol), nickel (II) iodide (0.2 g, 0.5 mmol) and (1R,2R)-2-aminocyclohexan-1-ol hydrochloride (0.08 g, 0.5 mmol) in i-PrOH (20 mL) under a nitrogen atmosphere. After stirring at 120° C. for 16 hours, the reaction mixture was cooled to room temperature, diluted with water (200 mL), and extracted with EA (3×200 mL). The combined organic layers were washed with brine (1 L), dried over anhydrous Na₂SO₄, filtered, concentrated under reduced pressure, and purified via flash column chromatography (Silica-CS (80 g) and eluting with EA in PE from 0% to 30% with 80 mL/min flow rate). Fractions with desired mass signal were combined and concentrated under vacuum to afford the product 1-(tetrahydro-2H-pyran-2-yl)-4-(tetrahydrofuran-3-yl)-1H-pyrazole (500 mg, 40% yield). LCMS (ESI-MS) m/z=223.1 [M+H]⁺.

### Step 2: 4-(tetrahydrofuran-3-yl)-1H-pyrazole

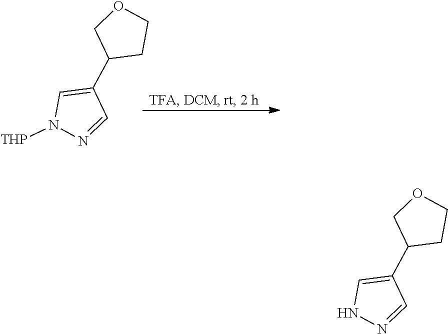

TFA (4 mL) was added to 1-(tetrahydro-2H-pyran-2-yl)-4-(tetrahydrofuran-3-yl)-1H-pyrazole (470 mg, 2.11 mmol) in DCM (20 mL). After stirring at room temperature for 2 hours. The resulting mixture was concentrated under reduced pressure and was dissolved in aq. Na₂CO₃ (20 mL). The resulting mixture was extracted with EA (3×20 mL) and the combined organic layers were washed with brine (100 mL), dried over anhydrous Na₂SO₄, filtered, and concentrated under reduced pressure to afford 4-(tetrahydrofuran-3-yl)-1H-pyrazole (300 mg crude). LCMS (ESI-MS) m/z=139.1 [M+H]⁺.

### Step 3: (E)-2⁵-chloro-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one

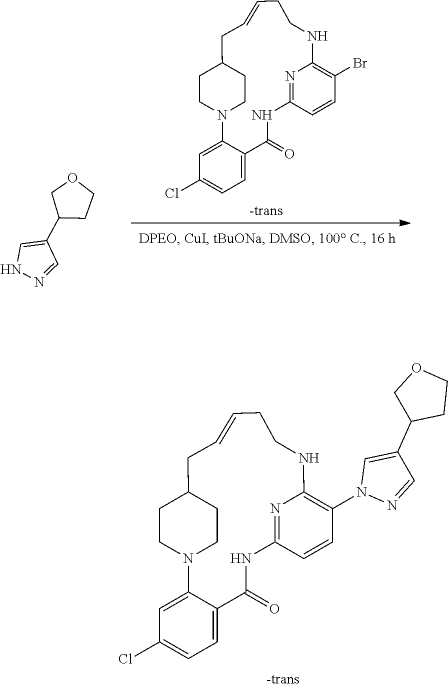 [SMILES: c1n[nH]cc1C1CCOC1 ; O=C1Nc2ccc(Br)c(n2)NCC/C=C/CC2CCN(CC2)c2cc(Cl)ccc21 ; O=C1Nc2ccc(-n3cc(C4CCOC4)cn3)c(n2)NCC/C=C/CC2CCN(CC2)c2cc(Cl)ccc21]

t-BuONa (30 mg, 0.32 mmol) was added to the mixture of 4-(tetrahydrofuran-3-yl)-1H-pyrazole (100 mg, 0.73 mmol), (E)-5⁵-bromo-2⁵-chloro-4,6-diaza-5(2,6)-pyridina-1(1,4)-piperidina-2(1,2)-benzenacyclounde-caphan-9-en-3-one (50 mg, 0.11 mmol), CuI (2.0 mg, 0.011 mmol) and N¹,N²-diphenethy-loxalamide (3.1 mg, 0.011 mol) in DMSO (5 mL) under a nitrogen atmosphere. After stirring at 100° C. for 16 hours, the reaction mixture was cooled to room temperature and was diluted with water (10 mL). The resulting mixture was extracted with EA (3×10 mL) and the combined organic layers were washed with brine (30 mL), dried over anhydrous Na₂SO₄, filtered, concentrated under reduced pressure and purified via reversed-phase flash chromatography with the following conditions: column, C18 silica gel; mobile phase, aq. 0.04% NH₄HCO₃ in ACN, 10% to 89% gradient in 10 min; detector, UV 254 nm to afford the product (E)-2⁵-chloro-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one (49 mg crude). LCMS (ESI-MS) m/z=533.1 [M+H]⁺.

### Step 4: (E)-N-(3-oxo-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-2⁵-yl) ethanesulfonamide

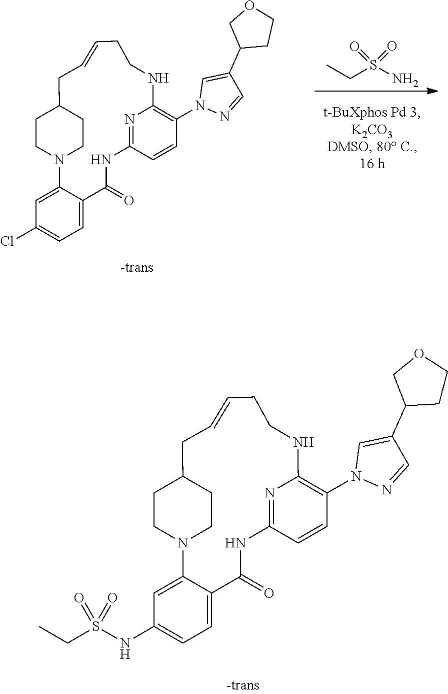

K₂CO₃ (31 mg, 0.23 mmol) was added to the mixture of (E)-2⁵-chloro-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-3-one (40 mg, 0.075 mmol), ethanesulfonamide (41 mg, 0.38 mmol) and t-BuXphos Pd G3 (7.9 mg, 0.075 mmol) in DMSO (12 mL) under a nitrogen atmosphere. After stirring at 80° C. for 16 hours, the reaction mixture was diluted with water (20 mL). The resulting mixture was extracted with EA (3×30 mL) and the combined organic layers were washed with brine (100 mL), dried over anhydrous Na₂SO₄, filtered, concentrated under reduced pressure, and purified by reversed-phase flash chromatography with the following conditions: column, C18 silica gel; mobile phase, aq. 0.04% NH₄HCO₃ in ACN, 10% to 100% gradient in 10 min; detector, UV 254 nm to afford the product (E)-N-(3-oxo-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (6 mg, 10% yield). LCMS (ESI-MS) m/z=606.3 [M+H]⁺.

Step 5: Compounds 626 and 627: (E)-N-(3-oxo-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (6 mg, 0.01 mmol) was separated by preparative chiral HPLC with the following conditions: Column: CHIRAL ART Cellulose SB; Mobile Phase A: Hex (0.2% FA): (IPA:DCM=1:1) =60:40; Flow rate: 1 mL/min mL/min; Gradient (B %); Injection Volume: 1 μL mL; Number Of Runs: 3 to afford the two products:

First eluting isomer (Compound 626): enantiopure (E)-N-(3-oxo-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-2⁵-yl) ethanesulfonamide (2.3 mg, 40% yield).

Second eluting isomer (Compound 627): enantiopure (S, E)-N-(3-oxo-5⁵-(4-(tetrahydrofuran-3-yl)-1H-pyrazol-1-yl)-4,6-diaza-5(2,6)-pyridina-1(1,4)-piperidina-2(1,2)-benzenacycloundecaphan-9-en-25-yl) ethanesulfonamide (2.3 mg, 40% yield).

### Compound 628.

K₃PO₄ (60 mg, 0.28 mmol) was added to the mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (50 mg, 0.09 mmol), 4-(1-methoxycyclopropyl)-1H-pyrazole (39 mg, 0.28 mmol), CuI (2 mg, 0.09 mmol) and (1R,2R)—N¹,N²-dimethylcyclohexane-1,2-diamine (2 mg, 0.09 mmol) in DMSO (2 mL) under a nitrogen atmosphere. The mixture was stirred at 100° C. overnight. The reaction mixture was purified by preparative HPLC to afford the product (E)-2⁵-(ethylsulfonyl)-5⁵-(4-(1-methoxycyclopropyl)-1H-pyrazol-1-yl)-4,6-diaza-5(2,6)-pyridina-1(1,4)-piperidina-2(1,2)-benzenacycloundecaphan-9-en-3-one (6.5 mg, 12% yield).

### Compound 630.

### Step 1: tert-butyl 4-(3-fluoro-1-((2-(trimethylsilyl) ethoxy) methyl)-1H-pyrazol-4-yl)-3,6-dihydropyridine-1(2H)-carboxylate

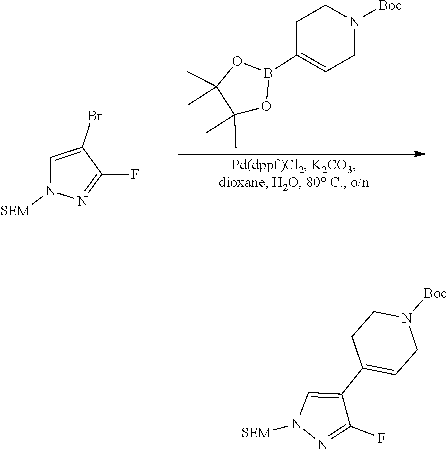 [SMILES: C[Si](C)(C)CCOCn1cc(Br)c(F)n1 ; CC(C)(C)OC(=O)N1CC=C(B2OC(C)(C)C(C)(C)O2)CC1 ; CC(C)(C)OC(=O)N1CC=C(c2cn(COCC[Si](C)(C)C)nc2F)CC1]

A solution of 4-bromo-3-fluoro-1-((2-(trimethylsilyl)ethoxy)methyl)-1H-pyrazole (100 mg, 0.34 mmol) in dioxane (0.9 mL) and water (0.3 mL) was charged with tert-butyl 4-(4,4,5,5-tetramethyl-1,3,2-dioxaborolan-2-yl)-3,6-dihydropyridine-1(2H)-carboxylate (126 mg, 0.41 mmol), K₂CO₃ (99.7 mg, 1.02 mmol) and Pd(dppf)Cl₂(24.8 mg, 0.034 mmol). The resulting solution was stirred at 80° C. for overnight. After cooling to room temperature, the reaction mixture was concentrated under reduced pressure and was purified with silica gel column chromatography (EA:PE=1:1) to afford tert-butyl 4-(3-fluoro-1-((2-(trimethylsilyl) ethoxy) methyl)-1H-pyrazol-4-yl)-3,6-dihydropyridine-1(2H)-carboxylate (110 mg, 65% yield). LCMS (ESI-MS) m/z=398.2 [M+H]⁺.

### Step 2: tert-butyl 4-(3-fluoro-1-((2-(trimethylsilyl) ethoxy) methyl)-1H-pyrazol-4-yl) piperidine-1-carboxylate

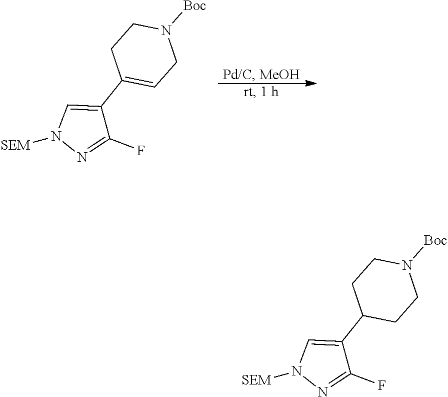 [SMILES: CC(C)(C)OC(=O)N1CC=C(c2cn(COCC[Si](C)(C)C)nc2F)CC1 ; CC(C)(C)OC(=O)N1CCC(c2cn(COCC[Si](C)(C)C)nc2F)CC1]

A solution of tert-butyl 4-(3-fluoro-1-((2-(trimethylsilyl) ethoxy) methyl)-1H-pyrazol-4-yl)-3,6-dihydropyridine-1(2H)-carboxylate (110 mg, 0.28 mmol) in MeOH (1.1 mL) was charged with Pd/C (Pd 10% on carbon, 11 mg) under a nitrogen atmosphere. The mixture was stirred at temperature for 1 hour. The reaction mixture was filtered and concentrated under reduced pressure to afford tert-butyl 4-(3-fluoro-1-((2-(trimethylsilyl) ethoxy) methyl)-1H-pyrazol-4-yl) piperidine-1-carboxylate (110 mg, 85% yield). LCMS (ESI-MS) m/z=400.2 [M+H]⁺.

### Step 3: tert-butyl 4-(3-fluoro-1H-pyrazol-4-yl) piperidine-1-carboxylate

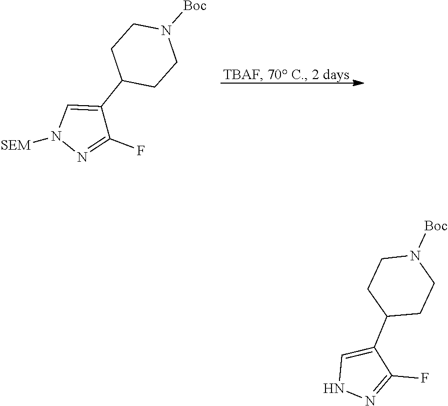 [SMILES: CC(C)(C)OC(=O)N1CCC(c2cn(COCC[Si](C)(C)C)nc2F)CC1 ; CC(C)(C)OC(=O)N1CCC(c2c[nH]nc2F)CC1]

2M TBAF in THF (1 mL) was added to tert-butyl 4-(3-fluoro-1-((2-(trimethylsilyl) ethoxy) methyl)-1H-pyrazol-4-yl) piperidine-1-carboxylate (90 mg, 0.23 mmol). The mixture was stirred at 70° C. for 2 days. The reaction mixture was diluted with NH₄Cl (aq.) (10 mL) and extracted with EA (10 mL). The combined organic layers were washed with NH₄Cl (aq.) (3×10 mL), dried over anhydrous Na₂SO₄, filtered, concentrated under reduced pressure, and the resulting crude product was purified by reversed-phase column chromatography (40 g, eluted with ACN in water (NH₄HCO₃ 0.1%) from 0% to 25%). Fractions with desired mass signal were combined and concentrated under vacuum to afford tert-butyl 4-(3-fluoro-1H-pyrazol-4-yl) piperidine-1-carboxylate (30 mg, 42% yield). LCMS (ESI-MS) m/z=268.15 [M−H]⁺.

### Step 4: tert-butyl (Z)-4-(1-(2⁵-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-3-fluoro-1H-pyrazol-4-yl) piperidine-1-carboxylate

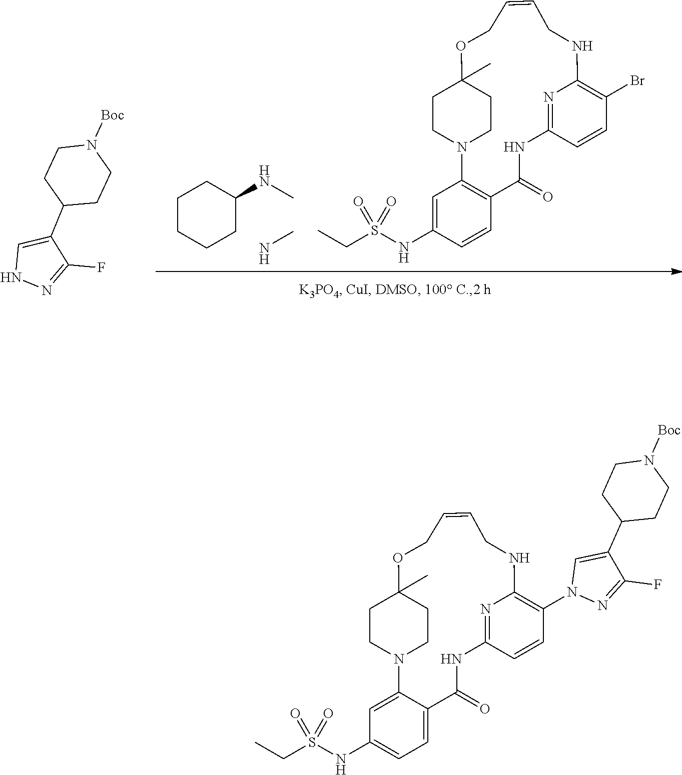

A mixture of (Z)—N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (30 mg, 0.05 mmol) and tert-butyl 4-(3-fluoro-1H-pyrazol-4-yl)piperidine-1-carboxylate (35 mg, 0.13 mmol) in DMSO (1 mL) and K₃PO₄ (34 mg, 0.16 mmol) were added (1R)—N¹,N²-dimethylcyclohexane-1,2-diamine (1.0 mg, 0.005 mmol) and CuI (1.0 mg, 0.005 mmol). After stirring for 2 hours at 100° C. under a nitrogen atmosphere, the mixture was allowed to cool to room temperature, was diluted with H₂O (3 mL) and was extracted with EA (3×3 mL). The combined organic layers were washed with brine (9 mL), dried over anhydrous Na₂SO₄, filtered, concentrated under reduced pressure, and the resulting crude product was purified by reversed-phase column chromatography (20 g, eluted with ACN in water (NH₄HCO₃ 0.1%) from 0% to 70%) to afford tert-butyl (Z)-4-(1-(2⁵-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-3-fluoro-1H-pyrazol-4-yl) piperidine-1-carboxylate (25 mg, 53% yield). LCMS (ESI-MS) m/z=753.40 [M+H]⁺.

### Step 5: (Z)—N-(5⁵-(3-fluoro-4-(piperidin-4-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide

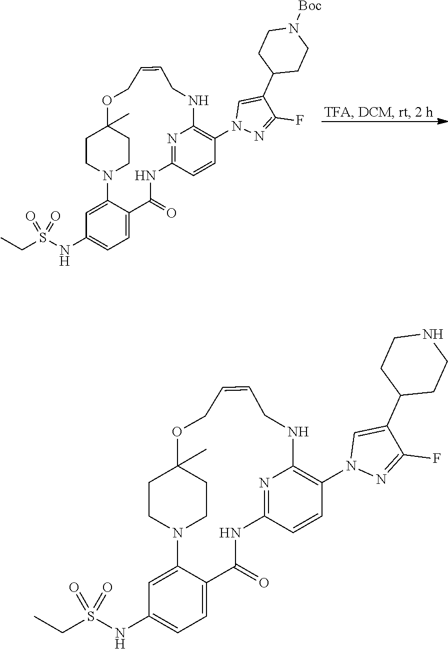 [SMILES: CCS(=O)(=O)Nc1ccc2c(c1)N1CCC(C)(CC1)OC/C=C\CNc1nc(ccc1-n1cc(C3CCN(C(=O)OC(C)(C)C)CC3)c(F)n1)NC2=O ; CCS(=O)(=O)Nc1ccc2c(c1)N1CCC(C)(CC1)OC/C=C\CNc1nc(ccc1-n1cc(C3CCNCC3)c(F)n1)NC2=O]

TFA (0.4 mL) was added to a mixture of tert-butyl (Z)-4-(1-(2⁵-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-3-fluoro-1H-pyrazol-4-yl) piperidine-1-carboxylate (25 mg, 0.03 mmol) in DCM (2 mL). The resulting mixture was concentrated under reduced pressure and the resulting crude product was purified by reversed-phase column chromatography (eluted with ACN in water (NH₄HCO₃ 0.1%) from 0% to 85%) to afford (Z)—N-(5⁵-(3-fluoro-4-(piperidin-4-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl) ethanesulfonamide (30 mg crude). LCMS (ESI-MS) m/z=653.35 [M+H]⁺.

Step 6: Compound 630: STAB (65 mg, 0.31 mmol) was added into a mixture of (Z)—N-(5⁵-(3-fluoro-4-(piperidin-4-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (20 mg, 0.03 mmol) and HCHO (1.0 mg, 0.03 mmol) in DCM (1 mL) at room temperature. The resulting mixture was stirred at room temperature for 1 hour then was quenched with solution of water (3 mL) and was extracted with DCM (3×3 mL). The combined organic layers were washed with brine (9 mL), dried with anhydrous Na₂SO₄, filtered, concentrated under reduced pressure, and the resulting crude product (30 mg) was purified by preparative HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 34% B to 64% B in 8 min; Wave Length: 254 nm/220 nm n; RT1 (min): 7.27) to afford (Z)—N-(5⁵-(3-fluoro-4-(1-methylpiperidin-4-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (11.1 mg, 54% yield).

### Compound 631.

1M TBAF in THF (9.5 mL, 9.5 mmol) was added to (E)-5⁵-bromo-2⁵-((2-((tert-butyldimethylsilyl) oxy) ethyl) sulfonyl)-4,6-diaza-5(2,6)-pyridina-1(1,4)-piperidina-2(1,2)-benzenacycloundecaphan-9-en-3-one (2.5 g, 3.8 mmol) in THF (25 mL). The mixture was stirred at room temperature for 2 hours. The reaction was poured into aq. NH₄Cl (50 mL) at temperature. The resulting mixture was extracted with EA (50 mL). The combined organic layers were washed with aq. NH₄Cl (2×50 mL), dried over anhydrous Na₂SO₄., filtered, concentrated under reduced pressure. The resulting crude product was purified by preparative TLC (eluted with MeOH:DCM (1:10)) to afford (E)-5⁵-bromo-2⁵-((2-hydroxyethyl) sulfonyl)-4,6-diaza-5(2,6)-pyridina-1(1,4)-piperidina-2(1,2)-benzenacycloundecaphan-9-en-3-one (1.21 g, 55% yield).

Compound 632 was prepared according to General Procedure 1 using Compound 389 and N,N-dimethyl-1H-pyrazole-4-carboxamide to afford (E)-1-(2⁵-(ethylsulfonamido)-1⁴-methyl-3-oxo-11-oxa-4,6-diaza-5(2,6)-pyridina-1(1,4)-piperidina-2(1,2)-benzenacycloundecaphan-8-en-5⁵-yl)-N,N-dimethyl-1H-pyrazole-4-carboxamide (2.4 mg, 8.8% yield).

Compound 633 was prepared according to General Procedure 1 using Compound 387 and (S)-3-hydroxypyrrolidine hydrochloride to afford (S,E)-N-(5⁵-(3-(3-hydroxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.2 mg, 29.1% yield).

Compound 634 was prepared according to General Procedure 1 using Compound 387 and 2-methyl-2-(1H-pyrazol-3-yl)propan-1-ol to afford (Z)—N-(5⁵-(3-(1-hydroxy-2-methylpropan-2-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.0 mg, 17.3% yield).

Compound 635 was prepared according to General Procedure 1 using Compound 390 and [1-(1H-pyrazol-3-yl)cyclopentyl]methanol to afford (Z)—N-(5⁵-(3-(1-(hydroxymethyl)cyclopentyl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.0 mg, 20.7% yield).

Compound 636 was prepared according to General Procedure 1 using Compound 390 and 4-piperidinol to afford (E)-N-(5⁵-(3-(4-hydroxypiperidin-1-yl)-1H-pyrazol-1-yl)-3-oxo-4,6-diaza-5(2,6)-pyridina-1(1,4)-piperidina-2(1,2)-benzenacycloundecaphan-9-en-2⁵-yl)ethanesulfonamide (2.1 mg, 12.9% yield).

Compound 637 was prepared according to the procedure used to prepare Compound 589 using Intermediate AA and 3-(bromomethyl)oxetane to afford (Z)-5⁵-bromo-1⁴-methyl-2⁵-((oxetan-3-ylmethyl)sulfonyl)-11-oxa-4,6-diaza-5(2,6)-pyridina-1(1,4)-piperidina-2(1,2)-benzenacycloundecaphan-8-en-3-one (12.3 mg, 31.1% yield).

Compound 638 was prepared according to General Procedure 1 using Compound 391 and (S)-tetrahydro-2H-pyran-3-ol to afford (S,E)-N-(3-oxo-5⁵-(3-((tetrahydro-2H-pyran-3-yl)oxy)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (2.3 mg, 7.8% yield).

Compound 639 was prepared according to General Procedure 1 using Compound 390 and (S)-3-fluoropiperidine hydrochloride to afford (S,Z)—N-(5⁵-(4-(3-fluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.4 mg, 2.4% yield).

Compound 640 was prepared according to General Procedure 1 using Compound 390 and (R)-3-methoxypyrrolidine to afford (R,Z)—N-(5⁵-(4-(3-methoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (4.6 mg, 24.2% yield).

Compound 641 was prepared according to General Procedure 1 using Compound 390 and 4-fluoropiperidine hydrochloride to afford (Z)—N-(5⁵-(4-(4-fluoropiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.4 mg, 1.9% yield).

Compound 642 was prepared according to General Procedure 1 using Compound 390 and 4-methoxypiperidine hydrochloride to afford (Z)—N-(5⁵-(4-(4-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (0.9 mg, 4.6% yield).

Compound 643 was prepared according to General Procedure 1 using Compound 390 and (R)-3-methoxypiperidine to afford (R,Z)—N-(5⁵-(4-(3-methoxypiperidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.0 mg, 30.8% yield).

Compound 644 was prepared according to General Procedure 1 using Compound 390 and (S)-(+)-3-fluoropyrrolidine hydrochloride to afford (S,Z)—N-(55-(4-(3-fluoropyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.2 mg, 7.3% yield).

Compound 645 was prepared according to General Procedure 1 using Compound 390 and 3,3-difluoropyrrolidine to afford (Z)—N-(55-(4-(3,3-difluoropyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (6.9 mg, 42.1% yield).

Compound 646 was prepared according to General Procedure 1 using Compound 390 and (S)-3-methoxypyrrolidine to afford (S,Z)—N-(5⁵-(4-(3-methoxypyrrolidin-1-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (5.9 mg, 30.7% yield).

Compound 647 was prepared according to General Procedure 1 using Compound 388 and N-(2-hydroxyethyl)pyrrolidine to afford (Z)—N-(1⁴-methyl-3-oxo-5⁵-(3-(2-(pyrrolidin-1-yl)ethoxy)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (2.9 mg, 17.6% yield).

Compound 648 was prepared according to General Procedure 1 using Compound 388 and 2-methyl-2-azaspiro[3.3]heptan-6-ol to afford (Z)—N-(1⁴-methyl-5⁵-(3-((2-methyl-2-azaspiro[3.3]heptan-6-yl)oxy)-1H-pyrazol-1-yl)-3-oxo-11-oxa-4,6-diaza-5(2,6)-pyridina-1(1,4)-piperidina-2(1,2)-benzenacycloundecaphanen-2⁵-yl)ethanesulfonamide (3 mg, 17.6% yield).

Compound 649 was prepared according to General Procedure 1 using Compound 387 and N-(2-hydroxyethyl)pyrrolidine to afford (E)-N-(1⁴-methyl-3-oxo-55-(3-(2-(pyrrolidin-1-yl)ethoxy)-1H-pyrazol-1-yl)-11-oxa-4,6-diaza-5(2,6)-pyridina-1(1,4)-piperidina-2(1,2)-benzenacycloundecaphan-8-en-25-yl)ethanesulfonamide (0.6 mg, 2.9% yield).

Compound 650 was prepared according to General Procedure 1 using Compound 388 and 1-cyclopropylazetidin-3-ol to afford (Z)—N-(55-(3-((1-cyclopropylazetidin-3-yl)oxy)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.2 mg, 16.5% yield).

Compound 651 was prepared according to General Procedure 1 using Compound 389 and N-methylcyclobutanamine hydrochloride to afford (E)-N-(5⁵-(4-(cyclopropyl(methyl)amino)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (1.3 mg, 8% yield).

Compound 652 was prepared according to General Procedure 2 using Compound 301 and 2-methyl-2-(1H-pyrazol-3-yl)propan-1-ol to afford (E)-2⁵-(ethylsulfonyl)-5⁵-(3-(1-hydroxy-2-methylpropan-2-yl)-1H-pyrazol-1-yl)-1⁴-methyl-11-oxa-4,6-diaza-5(2,6)-pyridina-1(1,4)-piperidina-2(1,2)-benzenacycloundecaphan-8-en-3-one (2.2 mg, 9.8% yield).

Compound 653 was prepared according to General Procedure 2 using Compound 311 and 2-methyl-2-(1H-pyrazol-3-yl)propan-1-ol to afford (E)-N-(5⁵-(3-(1-hydroxy-2-methylpropan-2-yl)-1H-pyrazol-1-yl)-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (3.2 mg, 14.1% yield).

Compound 654 was made using the procedure outlined in Step 6 of Compound 229.

Compound 655 was made using the procedure outlined in Compound 631 using (Z)-5⁵-bromo-2⁵-((2-((tert-butyldimethylsilyl)oxy)ethyl)sulfonyl)-1⁴-methyl-11-oxa-4,6-diaza-5(2,6)-pyridina-1(1,4)-piperidina-2(1,2)-benzenacycloundecaphan-8-en-3-one (2.5 g, 3.70 mmol) to afford (Z)-5⁵-bromo-2⁵-((2-hydroxyethyl)sulfonyl)-1⁴-methyl-11-oxa-4,6-diaza-5(2,6)-pyridina-1(1,4)-piperidina-2(1,2)-benzenacycloundecaphan-8-en-3-one (1.16 g, 55% yield).

### Compound 656.

### Step 1: 2-(1-((2-(trimethylsilyl) ethoxy) methyl)-1H-pyrazol-3-yl) acetonitrile

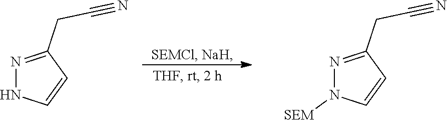 [SMILES: N#CCc1cc[nH]n1 ; C[Si](C)(C)CCOCn1ccc(CC#N)n1]

NaH (1.0 equiv) was added to 2-(1H-pyrazol-3-yl) acetonitrile (1.0 equiv) in THF (10 mL) at 0° C. The mixture was stirred at 0° C. for 0.5 hour, then SEM-Cl (1.0 equiv) was added to the reaction mixture at 0° C. After stirring at room temperature for 2 hours, the reaction mixture was diluted with aq. NH₄Cl (20 mL) and the resulting mixture was extracted with EA (3×20 V). The organic phases were combined, washed with brine (50 V), and the resulting mixture was concentrated under reduced pressure then purified via flash column chromatography (Silica-CS and eluting with EA in PE from 0% to 30%) to afford 2-(1-((2-(trimethylsilyl) ethoxy) methyl)-1H-pyrazol-3-yl) acetonitrile (38% yield). LCMS (ESI-MS) m/z=238.1 [M+H]⁺.

### Step 2: 1-(1H-pyrazol-3-yl) cyclopropane-1-carbonitrile

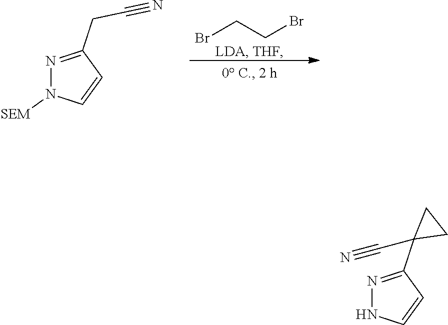

To a stirred mixture of 2-(1-((2-(trimethylsilyl)ethoxy)methyl)-1H-pyrazol-3-yl)acetonitrile (1.0 equiv) in THF (60 V) was added LDA (4.0 equiv) dropwise at 0° C. under a nitrogen atmosphere. The resulting mixture was stirred at 0° C. for 0.5 hour under nitrogen atmosphere, then 1,2-dibromoethane (1.1 equiv) was added dropwise at 0° C. The resulting mixture was stirred at 0° C. for additional 2 hours, then the reaction was quenched by the addition of aq. NH₄Cl (40 V) at 0° C. The resulting mixture was extracted with EA (3×40 V). The combined organic layers were washed with brine (100 V), dried over anhydrous Na₂SO₄, filtered, concentrated under reduced pressure, and purified by reversed-phase column chromatography (eluted with ACN in water (NH₄HCO₃ 0.1%) from 0% to 85%) a to afford 1-(1H-pyrazol-3-yl) cyclopropane-1-carbonitrile (25% yield).

### Step 3: Compound 656

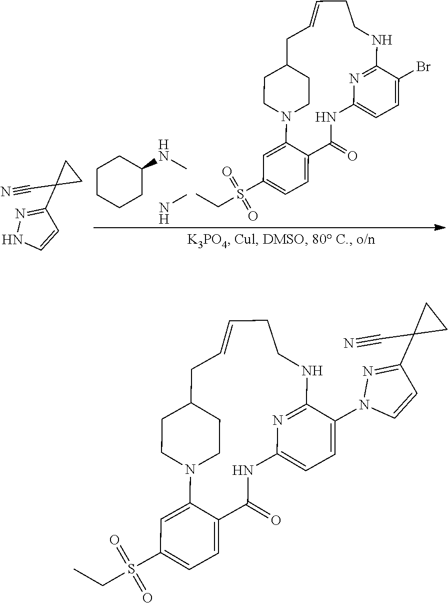

CuI (0.05 equiv) was added to a mixture of (E)-5⁵-bromo-2⁵-(ethylsulfonyl)-4,6-diaza-5(2,6)-pyridina-1(1,4)-piperidina-2(1,2)-benzenacycloundecaphan-9-en-3-one (1.0 equiv), 1-(1H-pyrazol-3-yl)cyclopropane-1-carbonitrile (4.0 equiv), (1R,2R)—N¹,N²-dimethylcyclohexane-1,2-diamine (0.05 equiv) and K₃PO₄ (4.0 equiv) in DMSO (20 V) under a nitrogen atmosphere. The mixture was stirred at 80° C. overnight. After cooling to room temperature, the resulting mixture was filtered and the filter cake was washed with DCM (3×20 V mL). The filtrate was concentrated under reduced pressure and the crude product was purified by reversed-phase column chromatography (eluted with ACN in water (NH₄HCO₃ 0.1%) from 0% to 90%) to afford the crude product. This material was further purified by preparative HPLC with the following conditions (Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min; Gradient (B %): 52% B to 82% B in 8 min; Wave Length: 254 nm/220 nm; RT1 (min): 7.13) to afford (E)-1-(1-(2⁵-(ethylsulfonyl)-3-oxo-4,6-diaza-5(2,6)-pyridina-1(1,4)-piperidina-2(1,2)-benzenacycloundecaphan-9-en-5⁵-yl)-1H-pyrazol-3-yl)cyclopropane-1-carbonitrile (14% yield).

### Compound 657.

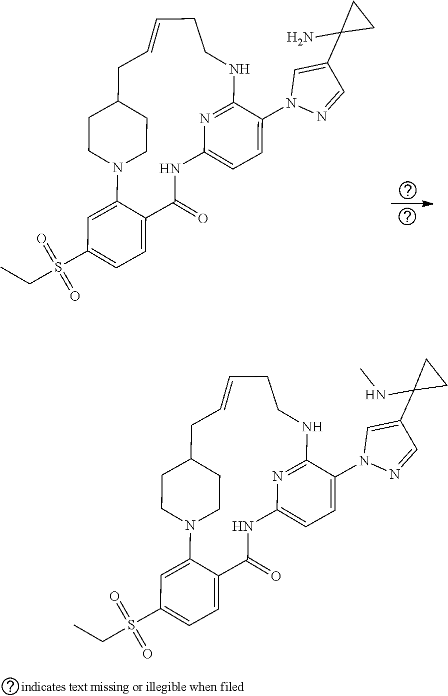 [SMILES: CCS(=O)(=O)c1ccc2c(c1)N1CCC(C/C=C/CCNc3nc(ccc3-n3cc(C4(N)CC4)cn3)NC2=O)CC1 ; CCS(=O)(=O)c1ccc2c(c1)N1CCC(C/C=C/CCNc3nc(ccc3-n3cc(C4(NC)CC4)cn3)NC2=O)CC1]

Pyrrolidine (40 mg, 0.56 mmol) was added into the mixture of (E)-55-(4-(1-aminocyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one (80 mg, 0.14 mmol) and formaldehyde (13 mg, 0.42 mmol) in HFIP (2.4 mL) at room temperature. The resulting mixture was stirred at room temperature for 3 days, then the mixture was quenched with water (10 mL), extracted with EA (3×10 mL), dried with anhydrous Na₂SO₄, filtered, and concentrated under reduced pressure. The crude product was purified by reversed-phase column (eluted with ACN in water (NH₄HCO₃ 0.1%) from 0% to 75%) to afford the crude product. The material was further purified by Prep-HPLC with following conditions: (Column: X-Bridge Prep Phenyl OBD C18 Column, 30*150 mm, 5 μm; Mobile Phase A: Water(0.1% FA), Mobile Phase B: ACN; Flow rate: 100 mL/min; Gradient (B %): 54% B to 84% B in 8 min; Wave Length: 254 nm/220 nm; RT1 (min): 7.59) and the desired fractions were combined and lyophilized to afford (E)-2⁵-(ethylsulfonyl)-5⁵-(4-(1-(methylamino)cyclopropyl)-1H-pyrazol-1-yl)-4,6-diaza-5(2,6)-pyridina-1(1,4)-piperidina-2(1,2)-benzenacycloundecaphan-9-en-3-one (7.8 mg, 9.5% yield).

### Compound 658.

### Step 1: (E)-N-(5⁵-(4-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamid

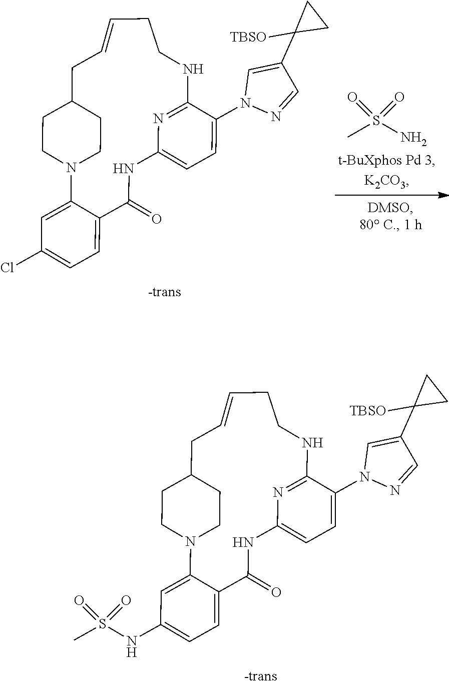

To a solution of (E)-5⁵-(4-(1-((tert-butyldimethylsilyl)oxy)cyclopropyl)-1H-pyrazol-1-yl)-2⁵-chloro-4,6-diaza-5(2,6)-pyridina-1(1,4)-piperidina-2(1,2)-benzenacycloundecaphan-9-en-3-one (30 mg, 0.05 mmol) and methanesulfonamide (6 mg, 0.06 mmol) in DMSO (1 mL) were added K₂CO₃ (20 mg, 0.14 mmol) and t-BuXphos Pd G3 (2 mg, 0.01 mmol). After stirring for 1 hour at 80° C. under a nitrogen atmosphere, the reaction mixture was allowed to cool to room temperature and was extracted with EA (3×5 mL). The combined organic layers were washed with water (15 mL), dried over anhydrous Na₂SO₄, filtered, concentrated under reduced pressure, and the resulting crude product was purified by reversed-phase column (eluted with ACN in water (NH₄HCO₃ 0.1%) from 0% to 65%) to afford (E)-N-(5⁵-(4-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (20 mg, 55% yield). LCMS (ESI-MS) m/z=692.2 [M+H]⁺.

Step 2: Compound 658: 1 M TBAF in THF (0.06 mL) was added into a mixture of (E)-N-(55-(4-(1-((tert-butyldimethylsilyl)oxy)cyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (15 mg, 0.02 mmol) in THF (3 mL) at room temperature. The resulting mixture was stirred at room temperature for 1 hour. After this time, the reaction mixture was diluted with NH₄Cl (aq.) (5 mL) and was extracted with EA (3×5 mL). The combined organic layers were washed with NH₄Cl (aq.) (15 mL), dried over anhydrous Na₂SO₄, filtered, and were concentrated under reduced pressure. The resulting crude product was purified by Prep-HPLC with the following conditions (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Wave Length: 254 nm/220 nm; RT1 (min): 7.43) to afford (E)-N-(5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-en-2⁵-yl)methanesulfonamide (1.9 mg, 15% yield).

### Compound 659 and 660.

### Step 1: Sodium 3-(4-allylpiperidin-1-yl)-4-(tert-butoxycarbonyl) benzenesulfinate (Compound 659) and (Z)—N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide (Compound 660)

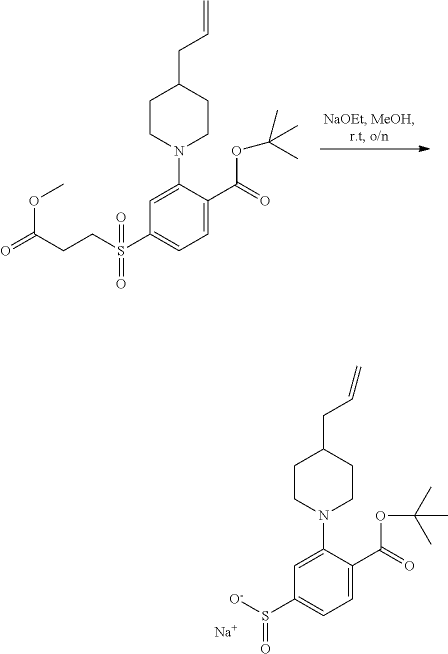 [SMILES: C=CCC1CCN(c2cc(S(=O)(=O)CCC(=O)OC)ccc2C(=O)OC(C)(C)C)CC1 ; [Na+] ; C=CCC1CCN(c2cc(S(=O)[O-])ccc2C(=O)OC(C)(C)C)CC1]

Sodium ethoxide (75.3 mg, 1.11 mmol) was added into a mixture of tert-butyl 2-(4-allylpiperidin-1-yl)-4-((3-methoxy-3-oxopropyl) sulfonyl) benzoate (500 mg, 1.11 mmol) in MeOH (5 mL) at room temperature. The resulting mixture was stirred overnight then concentrated under reduced pressure to afford crude product sodium 3-(4-allylpiperidin-1-yl)-4-(tert-butoxycarbonyl) benzenesulfinate (300 mg crude). LCMS (ESI-MS) m/z=366.1 [M+H]⁺.

### Step 2: tert-butyl 2-(4-allylpiperidin-1-yl)-4-(N-ethylsulfamoyl)benzoate

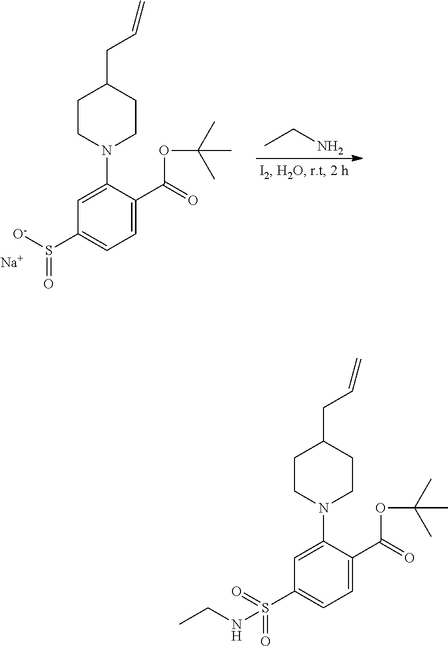

I₂ (328 mg, 1.29 mmol) was added into a mixture of sodium 3-(4-allylpiperidin-1-yl)-4-(tert-butoxycarbonyl)benzenesulfinate (500 mg, 1.29 mmol) and ethanamine (87.3 mg, 1.94 mmol) in H₂O (5 mL) at room temperature. The resulting mixture was stirred for 2 hours. The reaction mixture was diluted with water (10 mL) and was extracted with EA (3×10 mL). The combined organic layers were washed with brine (30 mL), dried over anhydrous Na₂SO₄, filtered, and concentrated under reduced pressure. The resulting crude product was was then purified via flash column chromatography (Silica-CS and eluting with EA in PE from 0% to 30%) to afford the desired product tert-butyl 2-(4-allylpiperidin-1-yl)-4-(N-ethylsulfamoyl)benzoate (230 mg, 42% yield). LCMS (ESI-MS) m/z=409.1 [M+H]⁺.

### Step 3: 2-(4-allylpiperidin-1-yl)-4-(N-ethylsulfamoyl)benzoic acid

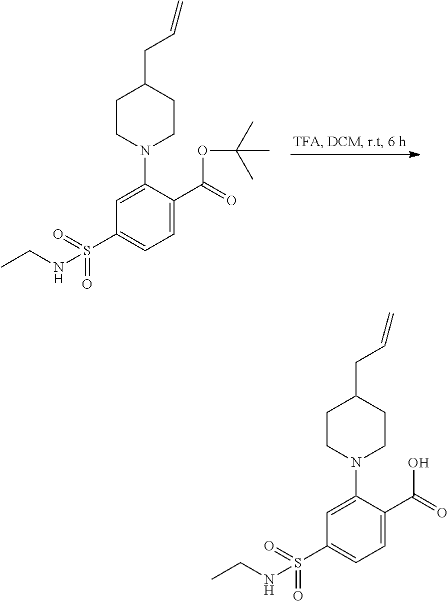

TFA (1.5 mL) was added into a mixture of tert-butyl 2-(4-allylpiperidin-1-yl)-4-(N-ethylsulfamoyl)benzoate (230 mg, 0.56 mmol) in DCM (3 mL) at room temperature and the reaction mixture was stirred for 6 hours. The resulting mixture was concentrated under reduced pressure to afford crude product 2-(4-allylpiperidin-1-yl)-4-(N-ethylsulfamoyl)benzoic acid (220 mg crude). LCMS (ESI-MS) m/z=352.9 [M+H]⁺.

### Step 4: 2-(4-allylpiperidin-1-yl)-N-(6-(but-3-en-1-ylamino)-5-(4-(1-((tert-butyldimethylsilyl) oxy) cyclopropyl)-1H-pyrazol-1-yl) pyridin-2-yl)-4-(N-ethylsulfamoyl) benzamide

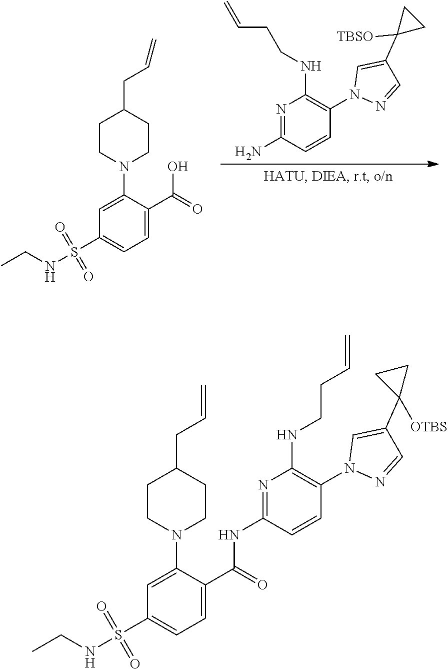 [SMILES: C=CCC1CCN(c2cc(S(=O)(=O)NCC)ccc2C(=O)O)CC1 ; C=CCCNc1nc(N)ccc1-n1cc(C2(O[Si](C)(C)C(C)(C)C)CC2)cn1 ; C=CCCNc1nc(NC(=O)c2ccc(S(=O)(=O)NCC)cc2N2CCC(CC=C)CC2)ccc1-n1cc(C2(O[Si](C)(C)C(C)(C)C)CC2)cn1]

DIEA (352 mg, 2.72 mmol) was added to a mixture of 2-(4-allylpiperidin-1-yl)-4-(N-ethylsulfamoyl)benzoic acid (320 mg, 0.9 mmol), N²-(but-3-en-1-yl)-3-(4-(1-((tert-butyldimethylsilyl)oxy)cyclopropyl)-1H-pyrazol-1-yl)pyridine-2,6-diamine (435 mg, 1.09 mmol) and HATU (518 mg, 1.36 mmol) in DMF (3 mL) at room temperature. The resulting mixture was stirred for 16 hours, then the residue was purified by reversed-phase flash chromatography with the following conditions: column, C18 silica gel; mobile phase, [H₂O] in [ACN], 10% to 90% gradient in 10 min; detector, UV 254 nm to afford 2-(4-allylpiperidin-1-yl)-N-(6-(but-3-en-1-ylamino)-5-(4-(1-((tert-butyldimethylsilyl)oxy)cyclopropyl)-1H-pyrazol-1-yl)pyridin-2-yl)-4-(N-ethylsulfamoyl)benzamide (300 mg, 43% yield). LCMS (ESI-MS) m/z=734.3 [M+H]⁺.

### Step 5: 55-(4-(1-((tert-butyldimethylsilyl)oxy)cyclopropyl)-1H-pyrazol-1-yl)-N-ethyl-3-oxo-4,6-diaza-5(2,6)-pyridina-1(1,4)-piperidina-2(1,2)-benzenacycloundecaphan-9-ene-2⁵-fonamide

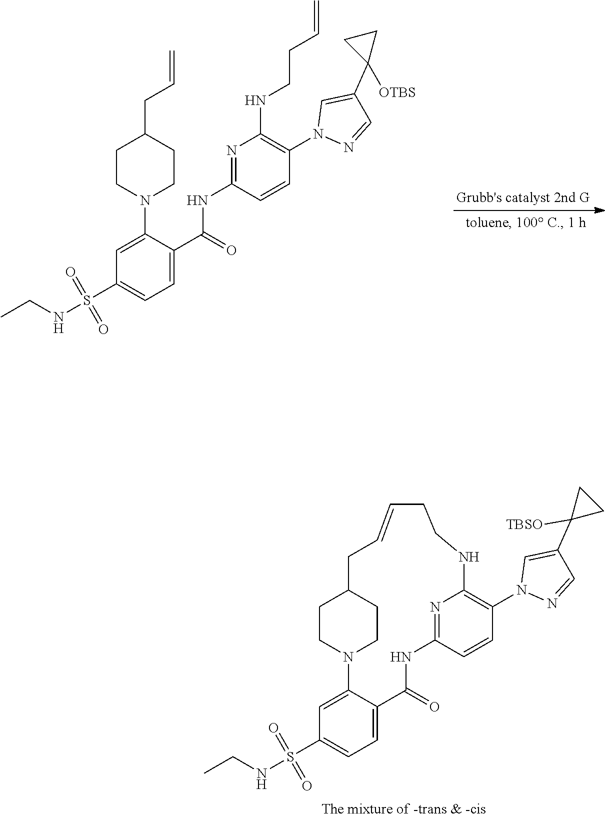 [SMILES: C=CCCNc1nc(NC(=O)c2ccc(S(=O)(=O)NCC)cc2N2CCC(CC=C)CC2)ccc1-n1cc(C2(O[Si](C)(C)C(C)(C)C)CC2)cn1 ; CCNS(=O)(=O)c1ccc2c(c1)N1CCC(C/C=C/CCNc3nc(ccc3-n3cc(C4(O[Si](C)(C)C(C)(C)C)CC4)cn3)NC2=O)CC1]

Grubb's catalyst 2nd Generation (32.4 mg, 0.04 mmol) was added to a mixture of 2-(4-allylpiperidin-1-yl)-N-(6-(but-3-en-1-ylamino)-5-(4-(1-((tert-butyldimethylsilyl)oxy) cyclopropyl)-1H-pyrazol-1-yl) pyridin-2-yl)-4-(N-ethylsulfamoyl) benzamide (280 mg, 0.38 mmol) in toluene (15 mL) at room temperature under a nitrogen atmosphere. The resulting mixture was stirred for 1 hour at 100° C. The resulting mixture was concentrated under reduced pressure, and the resulting crude product was purified via flash column chromatography (Silica-CS and eluting with EA in PE from 0% to 40%) to afford the desired product 5⁵-(4-(1-((tert-butyldimethylsilyl)oxy)cyclopropyl)-1H-pyrazol-1-yl)-N-ethyl-3-oxo-4,6-diaza-5(2,6)-pyridina-1(1,4)-piperidina-2(1,2)-benzenacycloundecaphan-9-ene-2⁵-sulfonamide (250 mg, 88% yield). LCMS (ESI-MS) m/z=706.3 [M+H]⁺.

### Step 6: N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide

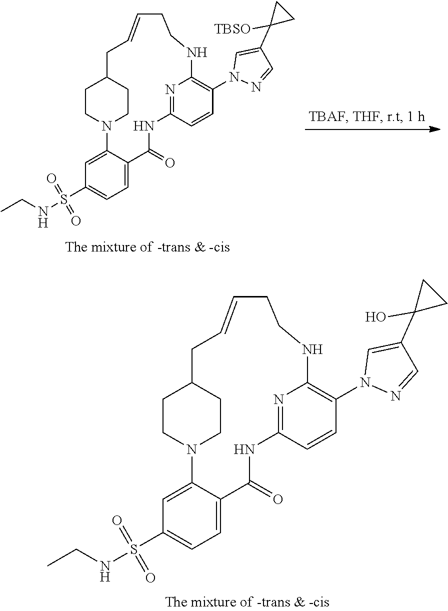 [SMILES: CCNS(=O)(=O)c1ccc2c(c1)N1CCC(C/C=C/CCNc3nc(ccc3-n3cc(C4(O[Si](C)(C)C(C)(C)C)CC4)cn3)NC2=O)CC1 ; CCNS(=O)(=O)c1ccc2c(c1)N1CCC(C/C=C/CCNc3nc(ccc3-n3cc(C4(O)CC4)cn3)NC2=O)CC1]

1M TBAF in THF (0.5 mL) was added to the stirred mixture of 5⁵-(4-(1-((tert-butyldimethylsilyl) oxy) cyclopropyl)-1H-pyrazol-1-yl)-N-ethyl-3-oxo-4,6-diaza-5(2,6)-pyridina-1(1,4)-piperidina-2(1,2)-benzenacycloundecaphan-9-ene-2⁵-sulfonamide (230 mg, 0.33 mmol) in THF (5 mL), then the resulting mixture was stirred for 1 hour at room temperature. After this time, the reaction mixture was diluted with NH₄Cl (3×3 mL) and was extracted with EA (3×3 mL). The combined organic layers were washed with brine (9 mL), dried over anhydrous Na₂SO₄, filtered, concentrated under reduced pressure, and the resulting crude product was purified by Prep-HPLC with the following conditions: Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 44% B to 74% B in 11 min; Wave Length: 254 nm/220 nm nm; RT1 (min): 7.28 to afford N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide (130 mg, 61% yield). LCMS (ESI-MS) m/z=592.1 [M+H]⁺.

Step 7: Compounds 659 and 660: N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide (130 mg, 0.22 mmol) was separated by Prep Chiral HPLC with the following condition: Column: (R, R)-WHELK-01, 5 μm, 250 mm×20 mm; Mobile Phase A: Hex (with 0.5% NH₃ (2 M in MeOH)), Mobile Phase B: MeOH:DCM=1:1; Flow rate: 20 mL/min; Gradient (B %) 80% B; Wave Length: 254/220 nm; RT1 (min): 4.09; RT2 (min): 6.99; Sample Solvent: EtOH; Injection Volume: 1.0 mL; Number Of Runs: 5; Single injection run time (min): 9 the desired fractions were combined and lyophilized to afford the two products:

First eluting isomer (Compound 660): (Z)—N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide (21.8 mg, 16.7% yield).

Second eluting isomer (Compound 659): (E)-N-ethyl-5⁵-(4-(1-hydroxycyclopropyl)-1H-pyrazol-1-yl)-3-oxo-4,6-diaza-5(2,6)-pyridina-1(1,4)-piperidina-2(1,2)-benzenacycloundecaphan-9-ene-2⁵-sulfonamide (63.8 mg, 48.8% yield).

### Compound 661.

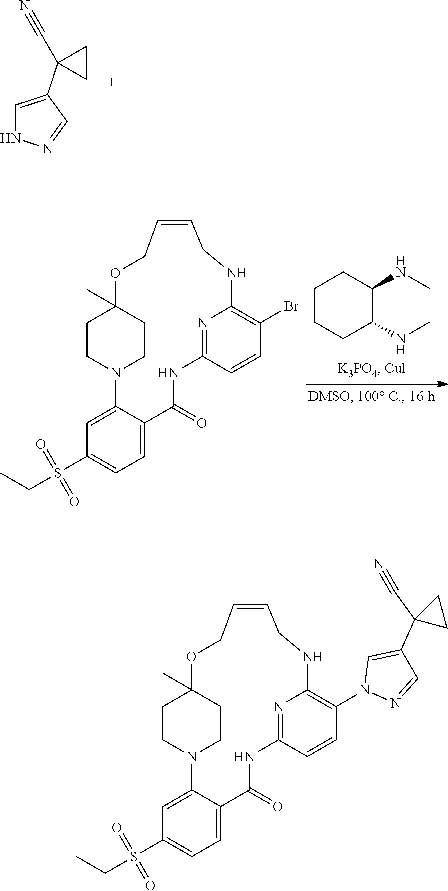

CuI (2 mg, 0.01 mmol) and (1R,2R)—N¹,N²-dimethylcyclohexane-1,2-diamine (2 mg, 0.01 mmol) were added into a mixture of (Z)-5⁵-bromo-2⁵-(ethylsulfonyl)-1⁴-methyl-11-oxa-4,6-diaza-5(2,6)-pyridina-1(1,4)-piperidina-2(1,2)-benzenacycloundecaphan-8-en-3-one (50 mg, 0.09 mmol), 1-(1H-pyrazol-4-yl)cyclopropane-1-carbonitrile (24 mg, 0.18 mmol) and K₃PO₄ (58 mg, 0.27 mmol) in DMSO (1 mL) at room temperature under nitrogen atmosphere. The resulting mixture was stirred for overnight at 100° C. After cooling to room temperature, the reaction was quenched with water (5 mL) and extracted with EA (3×5 mL). The combined organic layers were washed with brine, dried with Na₂SO₄, filtered, concentrated under reduced pressure, and the resulting residue was purified by silica gel column chromatography (eluted with PE:EA (1:1)) to afford crude product. This material was further purified by Prep-HPLC with the following conditions: Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 42% B to 72% B in 8 min; Wave Length: 254 nm/220 nm nm; RT1 (min): 7.33 to afford (Z)-1-(1-(2⁵-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)cyclopropane-1-carbonitrile (11.8 mg, 22% yield).

Compound 662 was prepared according the procedure outlined in Compound 661 using (E)-N-(5⁵-bromo-1⁴-methyl-3-oxo-11-oxa-4,6-diaza-5(2,6)-pyridina-1(1,4)-piperidina-2(1,2)-benzenacycloundecaphan-8-en-2⁵-yl)ethanesulfonamide (50 mg, 0.09 mmol) to afford (Z)-1-(1-(25-(ethylsulfonyl)-1⁴-methyl-3-oxo-11-oxa-4,6-diaza-5(2,6)-pyridina-1(1,4)-piperidina-2(1,2)-benzenacycloundecaphan-8-en-5⁵-yl)-1H-pyrazol-4-yl)cyclopropane-1-carbonitrile (11.8 mg, 22% yield).

Compound 663 was prepared according to General Procedure 2 using Compound 300 and 4-(1-(difluoromethyl)cyclopropyl)-1H-pyrazole to afford (E)-5⁵-(4-(1-(difluoromethyl)cyclopropyl)-1H-pyrazol-1-yl)-2⁵-(ethylsulfonyl)-4,6-diaza-5(2,6)-pyridina-1(1,4)-piperidina-2(1,2)-benzenacycloundecaphan-9-en-3-one.

Compound 664 was prepared by coupling 4-(1H-pyrazol-4-yl) morpholin-3-one with isomerically-pure Intermediate M using the procedure outlined in Step 3 of Compound 358. The resulting material was then cross-coupled to methanesulfonamide using General Procedure 1 to afford (E)-N-(3-oxo-5⁵-(4-(3-oxomorpholino)-1H-pyrazol-1-yl)-4,6-diaza-5(2,6)-pyridina-1(1,4)-piperidina-2(1,2)-benzenacycloundecaphan-9-en-2⁵-yl)methanesulfonamide.

Compounds 665 and 666 were prepared according to the procedure outlined for Compounds 659 and 660 using methylamine in Step 2 to afford: (E)-5⁵-(4-(1-hydroxycyclopropyl)-1H-pyrazol-1-yl)-N-methyl-3-oxo-4,6-diaza-5(2,6)-pyridina-1(1,4)-piperidina-2(1,2)-benzenacycloundecaphan-9-ene-2⁵-sulfonamide (Compound 665) and (Z)-5⁵-(4-(-hydroxycyclopropyl)-1H-pyrazol-1-yl)-N-methyl-3-oxo-4,6-diaza-5(2,6)-pyridina-1(1,4)-piperidina-2(1,2)-benzenacycloundecaphan-9-ene-2⁵-sulfonamide (Compound 666).

Compound 667 was prepared according to General Procedure 1 using Compound 389 and pyrrolidine-d₉ to afford (E)-N-(1⁴-methyl-3-oxo-5⁵-(4-(pyrrolidin-1-yl-d₈)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (0.8 mg, 4.3% yield).

Compound 668 was prepared according to General Procedure 1 using Compound 389 and piperidine-d₁₁ to afford (E)-N-(1⁴-methyl-3-oxo-5⁵-(4-(piperidin-1-yl-d₁₀)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (16.7 mg, 88% yield).

Compound 669 was prepared according to General Procedure 1 using Compound 389 and pyrrolidine-d₉ to afford (Z)—N-(1⁴-methyl-3-oxo-5⁵-(3-(pyrrolidin-1-yl-d₈)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (2.0 mg, 13% yield).

Compound 670 was prepared according to General Procedure 1 using Compound 388 and piperidine-d₁₁ to afford (Z)—N-(1⁴-methyl-3-oxo-5⁵-(3-(piperidin-1-yl-d₁₀)-1H-pyrazol-1-yl)-11-oxa-4,6-diaza-5(2,6)-pyridina-1(1,4)-piperidina-2(1,2)-benzenacycloundecaphan-8-en-2⁵-yl)ethanesulfonamide (6.6 mg, 35% yield).

Compound 671 was prepared according to General Procedure 1 using Compound 388 and piperidine-d₁₁ to afford (Z)—N-(3-oxo-55-(3-(piperidin-1-yl-d₁₀)-1H-pyrazol-1-yl)-4,6-diaza-5(2,6)-pyridina-1(1,4)-piperidina-2(1,2)-benzenacycloundecaphan-9-en-2⁵-yl)ethanesulfonamide (0.5 mg, 2.7% yield).

Compound 672 and 673 were prepared according to the procedure outlined for Compounds 659 and 660 using methylamine in Step 2 and 4-(1-(6-amino-2-(but-3-en-1-ylamino)pyridin-3-yl)-1H-pyrazol-4-yl)morpholin-3-one in Step 4 to afford (E)-N-methyl-3-oxo-5⁵-(4-(3-oxomorpholino)-1H-pyrazol-1-yl)-4,6-diaza-5(2,6)-pyridina-1(1,4)-piperidina-2(1,2)-benzenacycloundecaphan-9-ene-2⁵-sulfonamide (Compound 672) and (Z)—N-methyl-3-oxo-5⁵-(4-(3-oxomorpholino)-1H-pyrazol-1-yl)-4,6-diaza-5(2,6)-pyridina-1(1,4)-piperidina-2(1,2)-benzenacycloundecaphan-9-ene-2⁵-sulfonamide (Compound 673).

Compound 674 was prepared according to the procedure outlined for Compound 351 but with 2.0 equiv of TBAF and 1 h of stirring at RT to afford (E)-2⁵-(ethylsulfonyl)-5⁵-(4-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one.

Compounds 675 and 676 were prepared according to the procedure outlined for Compound 589 using 1-iodoethane-1,1,2,2,2-d₅ in Step 1 to afford a mixture of (E)-2⁵-((ethyl-2,2,2-d₃)sulfonyl)-55-(4-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one (Compound 675) and (E)-2⁵-((ethyl-d₅)sulfonyl)-5⁵-(4-(1-hydroxycyclopropyl)-1H-pyrazol-1-yl)-4,6-diaza-5(2,6)-pyridina-1(1,4)-piperidina-2(1,2)-benzenacycloundecaphan-9-en-3-one (Compound 676)

### II. Biological Data

### Example A-1: KIF18A ADP-Glo Assay

The purpose of KIF18A ADP-Glo assay is to evaluate the inhibition (% inhibition and IC50 values) of small molecule inhibitors by using a Luminescent based ADP-Glo assay. KIF18A catalyzes the production of ADP from ATP. ADP-Glo assay monitors ADP producing biochemical reactions. ADP-Glo is performed in 2 steps upon completion of ATPase reaction: a combined termination of ATPase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the ATPase activity. KIF18A was purchased from Cytoskeleton (CS—KF18). Typical reaction solutions (5 uL final reaction volume) contained 1% DMSO (±inhibitor), 10 mM MgCI2, 4 uM Taxol, 1000 uM ATP, 0.01% Pluronic F-68, 0.1 mg/ml pre-formed micro-tubules, and 1 nM enzyme complex in 15 mM HEPES buffer at pH 7. 5. The assay was initiated with the addition of ATP solution, following a 30-minute pre-incubation of enzyme and inhibitor at room temperature in the reaction mixture. The reaction was stopped after 24 hours at room temperature by the addition of 5 μL of ADP-GLO Reagent and 10 μL of Kinase Detection Reagent. A ClarioStar plate-reader (BMG) was used to read the luminescent signal. The IC50 determinations were made from a plot of the fractional velocity as a function of inhibitor concentration fit to the 4 parameters IC50 equation with the enzyme concentration as a variable using the GraphPad Prism (San Diego).

Data for representative compounds can be found in Table A, wherein IC₅₀ data in nM, values <100 are denoted at +++++; values from 100-499 are denoted as ++++; values from 500-999 are denoted as +++; values from 1000-4999 are denoted as ++; values of 5000 and greater are denoted as +.

TABLE A
KIF18A Biochemical data

| Cpd No. | KIF18A IC₅₀ |
|---|---|
| 1 | +++++ |
| 2 | +++++ |
| 3 | +++++ |
| 4 | +++++ |
| 5 | +++++ |
| 6 | +++++ |
| 7 | +++++ |
| 8 | +++++ |
| 9 | +++++ |
| 10 | +++++ |
| 11 | +++++ |
| 12 | +++++ |
| 13 | +++++ |
| 14 | +++++ |
| 15 | +++++ |
| 16 | +++++ |
| 17 | +++++ |
| 18 | +++++ |
| 19 | +++++ |
| 20 | +++++ |
| 21 | +++++ |
| 22 | +++++ |
| 23 | +++++ |
| 24 | +++++ |
| 25 | +++++ |
| 26 | +++++ |
| 27 | +++++ |
| 28 | +++++ |
| 29 | +++++ |
| 30 | +++++ |
| 31 | +++++ |
| 32 | +++++ |
| 33 | +++++ |
| 34 | +++++ |
| 35 | +++++ |
| 36 | +++++ |
| 37 | +++++ |
| 38 | +++++ |
| 39 | +++++ |
| 40 | ++++ |
| 41 | +++++ |
| 42 | +++++ |
| 43 | +++++ |
| 44 | ++++ |
| 45 | ++ |
| 46 | + |
| 47 | +++++ |
| 48 | +++++ |
| 49 | +++++ |
| 50 | +++++ |
| 51 | +++++ |
| 52 | +++++ |
| 53 | +++++ |
| 54 | +++++ |
| 55 | +++++ |
| 56 | +++++ |
| 57 | +++++ |
| 58 | +++++ |
| 59 | +++++ |
| 60 | +++++ |
| 61 | +++++ |
| 62 | +++++ |
| 63 | +++++ |
| 64 | +++++ |
| 65 | +++++ |
| 66 | +++++ |
| 67 | +++++ |
| 68 | +++++ |
| 69 | +++++ |
| 70 | +++++ |
| 71 | +++++ |
| 72 | ++++ |
| 73 | +++++ |
| 74 | +++++ |
| 75 | ++++ |
| 76 | +++ |
| 77 | +++++ |
| 78 | ++ |
| 79 | ++ |
| 80 | +++++ |
| 81 | ++++ |
| 82 | +++++ |
| 83 | ++++ |
| 84 | +++++ |
| 85 | +++++ |
| 86 | +++++ |
| 87 | +++++ |
| 88 | +++++ |
| 89 | +++++ |
| 90 | +++++ |
| 91 | +++++ |
| 92 | +++++ |
| 93 | +++++ |
| 94 | +++++ |
| 95 | +++++ |
| 96 | +++++ |
| 97 | +++++ |
| 98 | +++++ |
| 99 | +++++ |
| 100 | +++++ |
| 101 | +++++ |
| 102 | +++++ |
| 103 | +++++ |
| 104 | +++ |
| 105 | +++++ |
| 106 | +++++ |
| 107 | ++++ |
| 108 | ++++ |
| 109 | +++++ |
| 110 | +++++ |
| 111 | +++++ |
| 112 | +++++ |
| 113 | ++++ |
| 114 | ++++ |
| 115 | + |
| 116 | ++ |
| 117 | +++++ |
| 118 | +++++ |
| 119 | +++++ |
| 120 | +++++ |
| 121 | +++++ |
| 122 | +++++ |
| 123 | +++++ |
| 126 | ++++ |
| 129 | +++++ |
| 130 | +++++ |
| 131 | |
| 132 | |
| 134 | ++ |
| 135 | +++ |
| 136 | ++ |
| 137 | +++++ |
| 138 | +++++ |
| 139 | +++++ |
| 140 | +++++ |
| 141 | ++++ |
| 142 | +++++ |
| 143 | +++++ |
| 144 | ++++ |
| 145 | +++++ |
| 146 | +++++ |
| 147 | +++++ |
| 148 | +++++ |
| 149 | +++++ |
| 150 | +++++ |
| 151 | ++++ |
| 152 | +++++ |
| 153 | +++++ |
| 154 | +++++ |
| 155 | +++++ |
| 156 | +++++ |
| 157 | +++++ |
| 158 | +++++ |
| 159 | +++++ |
| 160 | +++++ |
| 161 | +++++ |
| 162 | +++++ |
| 163 | +++++ |
| 164 | +++++ |
| 165 | +++++ |
| 166 | +++++ |
| 167 | +++++ |
| 168 | +++++ |
| 169 | +++++ |
| 170 | +++++ |
| 171 | +++++ |
| 172 | +++++ |
| 173 | +++++ |
| 174 | +++++ |
| 175 | +++++ |
| 176 | +++++ |
| 177 | +++++ |
| 178 | +++++ |
| 179 | +++++ |
| 180 | +++++ |
| 181 | +++++ |
| 182 | +++++ |
| 183 | +++++ |
| 184 | +++++ |
| 185 | +++++ |
| 186 | +++++ |
| 187 | +++++ |
| 188 | ++++ |
| 189 | +++++ |
| 190 | +++++ |
| 191 | ++++ |
| 192 | +++++ |
| 193 | +++++ |
| 194 | +++++ |
| 195 | +++++ |
| 196 | +++++ |
| 197 | +++++ |
| 198 | +++++ |
| 199 | +++++ |
| 200 | +++++ |
| 201 | +++++ |
| 202 | +++++ |
| 203 | +++++ |
| 204 | +++++ |
| 205 | +++++ |
| 206 | +++++ |
| 207 | +++++ |
| 208 | ++++ |
| 209 | ++++ |
| 210 | ++++ |
| 211 | +++++ |
| 212 | +++++ |
| 213 | +++ |
| 214 | ++++ |
| 215 | ++++ |
| 216 | ++++ |
| 217 | +++++ |
| 218 | +++++ |
| 219 | +++++ |
| 220 | +++++ |
| 221 | ++++ |
| 222 | ++++ |
| 223 | +++++ |
| 224 | +++ |
| 225 | +++++ |
| 226 | ++++ |
| 227 | +++++ |
| 228 | ++++ |
| 229 | +++++ |
| 230 | +++++ |
| 231 | ++++ |
| 232 | +++++ |
| 233 | +++++ |
| 234 | ++++ |
| 235 | +++++ |
| 236 | +++++ |
| 237 | ++++ |
| 238 | +++++ |
| 239 | ++++ |
| 240 | ++++ |
| 241 | ++++ |
| 242 | ++++ |
| 243 | + |
| 244 | +++++ |
| 245 | ++++ |
| 246 | +++++ |
| 247 | +++ |
| 248 | ++++ |
| 249 | ++++ |
| 250 | +++++ |
| 251 | +++++ |
| 252 | +++++ |
| 253 | ++++ |
| 254 | ++++ |
| 255 | +++++ |
| 256 | +++++ |
| 257 | ++++ |
| 258 | +++++ |
| 259 | ++++ |
| 260 | ++++ |
| 261 | ++++ |
| 262 | +++++ |
| 263 | +++++ |
| 264 | +++++ |
| 265 | +++++ |
| 266 | +++++ |
| 267 | ++++ |
| 268 | +++++ |
| 269 | ++++ |
| 270 | +++++ |
| 271 | ++++ |
| 272 | +++++ |
| 273 | +++++ |
| 274 | +++++ |
| 275 | ++++ |
| 276 | +++++ |
| 277 | +++++ |
| 278 | +++++ |
| 279 | +++++ |
| 280 | +++++ |
| 281 | +++++ |
| 282 | +++++ |
| 283 | +++++ |
| 284 | ++++ |
| 285 | ++++ |
| 286 | ++++ |
| 287 | +++++ |
| 288 | +++++ |
| 289 | +++++ |
| 290 | +++++ |
| 291 | +++++ |
| 292 | +++++ |
| 293 | +++++ |
| 294 | ++++ |
| 295 | +++++ |
| 296 | ++++ |
| 297 | ++++ |
| 298 | +++++ |
| 299 | + |
| 300 | +++++ |
| 301 | +++++ |
| 302 | ++++ |
| 303 | ++++ |
| 304 | +++++ |
| 305 | ++++ |
| 306 | +++++ |
| 307 | ++++ |
| 308 | +++++ |
| 309 | +++++ |
| 310 | +++++ |
| 311 | +++++ |
| 312 | +++++ |
| 313 | +++++ |
| 314 | +++++ |
| 315 | ++++ |
| 316 | ++++ |
| 317 | +++++ |
| 318 | +++++ |
| 319 | +++++ |
| 320 | ++++ |
| 321 | +++++ |
| 322 | ++++ |
| 323 | +++++ |
| 324 | +++++ |
| 325 | +++++ |
| 326 | ++++ |
| 327 | +++++ |
| 328 | ++++ |
| 329 | ++++ |
| 330 | +++++ |
| 331 | +++++ |
| 332 | ++++ |
| 333 | ++++ |
| 334 | +++++ |
| 335 | +++++ |
| 336 | ++++ |
| 337 | +++++ |
| 338 | +++++ |
| 339 | +++++ |
| 340 | ++++ |
| 341 | +++++ |
| 342 | ++++ |
| 343 | +++ |
| 344 | ++++ |
| 345 | ++++ |
| 346 | ++ |
| 347 | ++ |
| 348 | +++++ |
| 349 | ++++ |
| 350 | ++++ |
| 351 | +++++ |
| 352 | +++++ |
| 353 | +++++ |
| 354 | +++++ |
| 355 | ++++ |
| 356 | ++ |
| 357 | ++ |
| 358 | ++++ |
| 359 | +++++ |
| 360 | +++++ |
| 361 | +++++ |
| 362 | +++++ |
| 363 | +++++ |
| 364 | +++++ |
| 365 | +++++ |
| 366 | ++++ |
| 367 | +++++ |
| 368 | ++++ |
| 369 | ++++ |
| 370 | ++++ |
| 371 | ++++ |
| 372 | +++++ |
| 373 | +++++ |
| 374 | +++++ |
| 375 | +++++ |
| 376 | +++++ |
| 377 | +++++ |
| 378 | +++++ |
| 379 | +++++ |
| 380 | ++++ |
| 381 | +++++ |
| 382 | +++++ |
| 383 | +++++ |
| 384 | +++++ |
| 385 | +++++ |
| 386 | +++++ |
| 387 | +++++ |
| 388 | +++++ |
| 389 | +++++ |
| 390 | +++++ |
| 391 | +++++ |
| 392 | +++++ |
| 393 | +++++ |
| 394 | +++++ |
| 395 | +++++ |
| 396 | +++++ |
| 397 | +++++ |
| 398 | +++++ |
| 399 | ++ |
| 400 | +++++ |
| 401 | +++++ |
| 402 | +++++ |
| 403 | +++++ |
| 404 | +++++ |
| 405 | +++++ |
| 406 | + |
| 407 | ++++ |
| 408 | ++ |
| 409 | +++++ |
| 410 | ++++ |
| 411 | ++ |
| 412 | +++++ |
| 413 | ++++ |
| 414 | ++++ |
| 415 | ++++ |
| 416 | ++++ |
| 417 | +++++ |
| 418 | +++++ |
| 419 | +++++ |
| 420 | ++++ |
| 421 | +++++ |
| 422 | +++++ |
| 423 | ++++ |
| 424 | ++++ |
| 425 | +++++ |
| 426 | +++++ |
| 427 | +++++ |
| 428 | +++++ |
| 429 | ++++ |
| 430 | ++++ |
| 431 | +++++ |
| 432 | +++++ |
| 433 | ++++ |
| 434 | +++++ |
| 435 | +++++ |
| 436 | ++++ |
| 437 | +++ |
| 438 | +++ |
| 439 | ++ |
| 440 | ++ |
| 441 | +++ |
| 442 | ++++ |
| 443 | ++++ |
| 444 | ++++ |
| 445 | +++++ |
| 446 | +++++ |
| 447 | ++++ |
| 448 | +++++ |
| 449 | ++ |
| 450 | ++++ |
| 451 | ++++ |
| 452 | ++++ |
| 453 | +++ |
| 454 | ++ |
| 455 | +++++ |
| 456 | +++++ |
| 457 | ++++ |
| 458 | ++++ |
| 459 | +++++ |
| 460 | ++ |
| 461 | ++++ |
| 462 | +++++ |
| 463 | +++++ |
| 464 | +++++ |
| 465 | +++++ |
| 466 | +++ |
| 467 | ++++ |
| 468 | +++++ |
| 469 | +++++ |
| 470 | ++++ |
| 471 | ++++ |
| 472 | ++++ |
| 473 | ++ |
| 474 | +++++ |
| 475 | ++ |
| 476 | ++++ |
| 477 | ++ |
| 478 | +++++ |
| 479 | ++++ |
| 480 | ++++ |
| 481 | ++++ |
| 482 | +++++ |
| 483 | +++++ |
| 484 | +++++ |
| 485 | ++++ |
| 486 | +++++ |
| 487 | +++++ |
| 488 | +++++ |
| 489 | +++++ |
| 490 | +++++ |
| 491 | ++++ |
| 492 | +++++ |
| 493 | +++++ |
| 494 | +++++ |
| 495 | ++++ |
| 496 | ++++ |
| 497 | ++++ |
| 498 | +++++ |
| 499 | +++++ |
| 500 | +++++ |
| 501 | ++++ |
| 502 | ++++ |
| 503 | +++++ |
| 504 | ++++ |
| 505 | ++++ |
| 506 | +++++ |
| 507 | +++++ |
| 508 | ++++ |
| 509 | +++++ |
| 510 | ++++ |
| 511 | ++++ |
| 512 | +++++ |
| 513 | +++++ |
| 514 | ++++ |
| 515 | +++++ |
| 516 | +++++ |
| 517 | +++++ |
| 518 | ++++ |
| 519 | +++++ |
| 520 | ++++ |
| 521 | +++++ |
| 522 | +++++ |
| 523 | +++++ |
| 524 | +++++ |
| 525 | +++++ |
| 526 | ++++ |
| 527 | +++++ |
| 528 | ++++ |
| 529 | +++++ |
| 530 | +++++ |
| 531 | ++++ |
| 532 | +++++ |
| 533 | +++++ |
| 534 | ++++ |
| 535 | +++++ |
| 536 | +++++ |
| 537 | ++ |
| 538 | +++++ |
| 539 | +++++ |
| 540 | +++++ |
| 541 | +++++ |
| 542 | +++++ |
| 543 | ++++ |
| 544 | +++++ |
| 545 | ++++ |
| 546 | +++++ |
| 547 | ++++ |
| 548 | ++ |
| 549 | ++ |
| 550 | + |
| 551 | + |
| 552 | ++++ |
| 553 | ++++ |
| 554 | ++++ |
| 555 | ++ |
| 556 | ++++ |
| 557 | +++ |
| 558 | ++++ |
| 559 | ++++ |
| 560 | +++++ |
| 561 | +++++ |
| 562 | +++++ |
| 563 | +++++ |
| 564 | +++++ |
| 565 | ++++ |
| 566 | ++++ |
| 567 | ++++ |
| 568 | ++++ |
| 569 | +++++ |
| 570 | ++++ |
| 571 | +++++ |
| 572 | ++++ |
| 573 | ++++ |
| 574 | +++++ |
| 575 | ++++ |
| 576 | +++ |
| 577 | +++++ |
| 578 | ++++ |
| 579 | +++++ |
| 580 | ++++ |
| 581 | ++++ |
| 582 | +++++ |
| 583 | ++++ |
| 584 | +++++ |
| 585 | +++ |
| 586 | ++++ |
| 587 | +++++ |
| 588 | +++++ |
| 589 | +++++ |
| 590 | +++++ |
| 591 | +++ |
| 592 | +++ |
| 593 | +++ |
| 594 | ++++ |
| 595 | +++++ |
| 596 | ++++ |
| 597 | +++++ |
| 598 | +++++ |
| 599 | ++++ |
| 600 | ++++ |
| 601 | ++++ |
| 602 | +++++ |
| 603 | ++++ |
| 604 | +++++ |
| 605 | +++++ |
| 606 | ++++ |
| 607 | +++ |
| 608 | ++ |
| 609 | +++++ |
| 610 | +++++ |
| 611 | ++++ |
| 612 | ++++ |
| 613 | ++++ |
| 614 | ++++ |
| 615 | +++++ |
| 616 | ++ |
| 617 | ++++ |
| 618 | ++++ |
| 619 | ++++ |
| 620 | +++++ |
| 621 | +++++ |
| 622 | +++++ |
| 623 | +++++ |
| 624 | ++++ |
| 625 | +++++ |
| 626 | +++++ |
| 627 | +++++ |
| 628 | +++++ |
| 629 | +++++ |
| 630 | +++++ |
| 631 | +++++ |
| 632 | +++++ |
| 633 | +++++ |
| 634 | +++++ |
| 635 | ++++ |
| 636 | +++++ |
| 637 | ++++ |
| 638 | ++++ |
| 639 | ++++ |
| 640 | ++++ |
| 641 | ++++ |
| 642 | +++ |
| 643 | ++++ |
| 644 | ++++ |
| 645 | +++++ |
| 646 | ++++ |
| 647 | +++++ |
| 648 | +++++ |
| 649 | ++++ |
| 650 | ++++ |
| 651 | ++++ |
| 652 | ++++ |
| 653 | ++++ |
| 654 | +++++ |
| 655 | +++++ |
| 656 | +++++ |
| 657 | ++++ |
| 658 | +++++ |
| 659 | +++++ |
| 660 | +++++ |
| 661 | +++++ |
| 662 | +++++ |
| 663 | +++++ |
| 664 | +++++ |
| 665 | +++++ |
| 666 | +++++ |
| 667 | ++++ |
| 668 | ++++ |
| 669 | +++++ |
| 670 | ++++ |
| 671 | ++ |

### Example A-2: CYP Inhibition Assay

The purpose of the single point Cytoclirome P450 Drug-Drug Interaction (DDJ) assay is to evaluate the inhibition (% inhibition) of small molecule inhibitors on a panel of Cytochrome P450 metabolic enzyme isoforms (CYPs 2B36, 2C8, 2C9, 2C19, 2D6, and 3A4). To determine if a small molecule is a potential CYP inhibitor, the compound is incubated (at a concentration of 10 uM) with human liver microsomes at 0.2 mg/mL containing 20 mM NADPH. First, the test compound was incubated in the above mixture for 10 minutes at 37° C. After that a mixture of CYP substrates (Bupropion (2B36) 2 μM, Amodiaquine (2C8) 1 μM, Tolbutamide(2C9) 25 μM, Mephenytoin (2C19) 50 μM, Dextromethorphan (2D6) 10 μM and Midazolam (3A4) 2 μM) was added and incubated at 37° C. for an additional 15 minutes. The reaction was then quenched with 1:1 addition of acetonitrile containing 0.1 μM Verapamil (as internal standard/IS). The samples were centrifuged at 4200 g for 15 minutes to precipitate protein. Then diluted 1:1 ultra-pure H₂O, and the mixture was analyzed by LC-MS/MS. Formation of the substate metabolites Hydroxybupropion, Desethylamodiaquine. Hydroxytolbutamide, Hydroxymephenytoin, Dextrorphan, and Hydroxymidazolam were monitored using MRM with a Triple Quad™ 6500+ instrument from AB Sciex with an ESI interface. The % inhibition for each CYP were calculated by comparing the amount of substrate metabolite generated for the test compound incubation to the DMSO control incubation using the formula: 1−(Compound/DMSO Control). 100015101 Data for representative compounds can be found in Table B, wherein “A” represents a value of 0-50% inhibition and “B” represents a value of 50-100% inhibition.

TABLE B
Cyp % Inhibition Data

| Cpd. No | CYP2B6 | CYP2C9 | CYP2D6 | CYP2C8 | CYP3A4 | CYP2C19 |
|---|---|---|---|---|---|---|
| 1 | A | A | A | A | A | A |
| 2 | A | A | A | A | A | A |
| 3 | A | A | A | A | A | A |
| 4 | A | B | A | A | A | A |
| 5 | A | B | A | B | A | A |
| 6 | A | A | A | A | A | A |
| 7 | A | B | A | A | A | A |
| 8 | A | B | A | A | A | A |
| 9 | A | B | A | A | A | A |
| 10 | A | A | A | A | A | A |
| 11 | A | A | A | A | A | A |
| 12 | A | A | A | A | A | A |
| 13 | A | A | A | A | A | A |
| 14 | A | A | A | A | A | A |
| 15 | A | A | A | A | A | A |
| 16 | A | A | A | A | A | A |
| 17 | A | A | A | A | A | A |
| 18 | A | A | A | A | A | A |
| 19 | A | B | A | B | B | B |
| 20 | A | B | A | B | B | B |
| 21 | A | A | A | A | A | A |
| 22 | A | B | A | A | A | A |
| 23 | A | B | A | A | A | A |
| 24 | A | A | A | A | A | A |
| 25 | A | A | A | A | A | A |
| 26 | A | A | A | A | A | A |
| 27 | A | A | A | A | A | A |
| 28 | A | A | A | A | A | A |
| 29 | A | B | A | A | A | A |
| 30 | A | A | A | A | A | A |
| 31 | A | A | A | A | A | A |
| 32 | A | A | A | A | A | A |
| 33 | A | A | A | A | A | A |
| 34 | A | A | A | A | A | A |
| 35 | A | B | A | A | A | A |
| 36 | A | A | A | A | A | A |
| 37 | A | B | A | A | A | A |
| 38 | A | A | A | A | A | A |
| 39 | A | A | A | A | A | A |
| 40 | A | A | A | A | A | A |
| 41 | A | A | A | A | A | A |
| 42 | A | A | A | A | A | A |
| 43 | A | A | A | A | A | A |
| 44 | B | A | A | A | B | A |
| 45 | A | A | A | A | A | A |
| 46 | A | A | A | A | A | A |
| 49 | A | A | A | A | A | A |
| 50 | A | A | A | A | A | A |
| 51 | A | A | A | B | A | A |
| 52 | A | B | A | A | A | A |
| 53 | A | B | A | A | A | A |
| 54 | A | B | A | A | B | A |
| 55 | A | B | A | A | A | A |
| 56 | A | A | A | A | A | A |
| 57 | A | A | A | A | A | A |
| 58 | A | A | A | A | A | A |
| 59 | A | A | A | A | A | A |
| 60 | A | B | A | A | A | A |
| 61 | A | B | A | A | A | A |
| 62 | A | A | A | A | A | A |
| 63 | A | B | A | A | A | A |
| 64 | A | B | A | B | A | A |
| 65 | A | B | A | A | A | A |
| 66 | A | B | A | A | A | A |
| 67 | A | B | A | A | A | A |
| 68 | A | B | A | B | A | A |
| 69 | A | A | A | B | A | A |
| 70 | A | A | A | A | A | A |
| 71 | B | B | A | B | B | B |
| 72 | A | B | A | A | A | A |
| 73 | A | B | A | A | A | A |
| 74 | A | B | A | A | A | A |
| 75 | A | B | A | A | A | A |
| 76 | A | B | A | A | A | A |
| 77 | A | B | A | A | A | A |
| 78 | A | A | A | A | A | A |
| 79 | A | A | A | A | A | A |
| 80 | A | B | A | A | A | A |
| 81 | A | B | A | A | A | A |
| 82 | A | B | A | B | A | A |
| 83 | A | A | A | A | A | A |
| 84 | A | A | A | B | A | A |
| 85 | A | A | A | B | A | A |
| 86 | A | A | A | B | A | A |
| 87 | A | A | A | B | A | A |
| 88 | A | A | A | B | A | A |
| 89 | A | A | A | A | A | A |
| 90 | A | A | A | B | A | A |
| 91 | A | A | A | B | A | A |
| 92 | A | B | A | B | A | A |
| 93 | A | A | A | B | A | A |
| 94 | A | A | A | A | A | A |
| 95 | A | A | A | B | A | A |
| 96 | A | A | A | B | A | A |
| 97 | A | B | A | B | A | A |
| 98 | A | A | A | A | A | A |
| 99 | A | A | A | A | A | A |
| 100 | A | A | A | A | A | A |
| 101 | A | B | A | A | A | A |
| 102 | A | B | A | A | A | A |
| 103 | A | B | A | A | A | A |
| 104 | A | A | A | A | A | A |
| 105 | A | B | A | A | A | A |
| 106 | A | A | A | A | A | A |
| 107 | A | A | A | A | A | A |
| 108 | A | A | A | A | A | A |
| 109 | A | B | A | A | A | A |
| 110 | A | B | A | A | A | A |
| 111 | A | B | A | A | A | A |
| 112 | A | B | A | A | A | B |
| 113 | A | A | A | A | A | A |
| 114 | A | A | A | A | A | A |
| 115 | A | A | A | A | A | A |
| 116 | A | A | A | A | A | A |
| 117 | A | A | A | A | A | A |
| 118 | A | B | A | A | A | A |
| 119 | A | B | A | A | A | B |
| 120 | A | A | A | B | A | A |
| 121 | A | B | A | B | A | A |
| 122 | A | B | A | B | A | B |
| 123 | A | B | A | A | A | A |
| 124 | A | B | A | A | A | A |
| 125 | A | A | A | A | A | A |
| 126 | A | A | A | A | A | A |
| 127 | A | A | A | A | A | A |
| 128 | A | B | A | A | A | A |
| 129 | A | A | A | A | A | A |
| 130 | A | B | A | A | A | A |
| 131 | A | B | A | A | A | A |
| 132 | A | B | A | A | A | A |
| 137 | A | A | A | A | A | A |
| 138 | A | A | A | A | A | A |
| 139 | A | A | A | A | A | A |
| 140 | A | A | A | A | A | A |
| 141 | A | A | A | A | A | A |
| 142 | A | A | A | A | A | A |
| 143 | A | A | A | A | A | A |
| 144 | A | A | A | A | A | A |
| 145 | A | A | A | A | A | A |
| 146 | A | B | A | A | A | A |
| 147 | A | B | A | A | A | A |
| 148 | A | B | A | A | A | A |
| 149 | A | A | A | A | A | A |
| 150 | A | A | A | A | A | A |
| 151 | A | A | A | A | A | A |
| 152 | A | A | A | A | A | A |
| 153 | A | B | A | A | A | A |
| 154 | A | A | A | B | A | A |
| 155 | A | A | A | A | A | A |
| 156 | A | B | A | A | A | A |
| 157 | A | A | A | A | A | A |
| 158 | A | A | A | A | A | A |
| 159 | A | A | A | A | A | A |
| 160 | A | A | A | A | A | A |
| 161 | A | B | A | A | A | A |
| 162 | A | A | A | A | A | A |
| 163 | A | A | A | A | A | A |
| 164 | A | A | A | A | A | A |
| 165 | A | B | A | A | A | A |
| 166 | A | B | A | A | A | A |
| 167 | A | B | A | A | A | A |
| 168 | A | B | A | A | A | A |
| 169 | A | B | A | A | A | B |
| 170 | A | A | A | A | A | A |
| 171 | A | B | A | A | A | A |
| 172 | B | B | A | B | A | A |
| 173 | A | A | A | A | A | A |
| 174 | A | B | A | B | A | A |
| 175 | A | B | A | A | A | A |
| 176 | A | B | A | A | A | A |
| 177 | A | C | A | A | B | B |
| 178 | A | B | A | A | A | A |
| 207 | A | A | A | A | A | A |
| 208 | A | A | A | A | A | A |
| 209 | A | A | A | A | B | A |
| 210 | A | A | A | A | A | B |
| 211 | A | A | A | A | A | A |
| 213 | A | A | A | A | A | A |
| 214 | A | A | A | A | A | A |
| 215 | A | A | A | A | A | A |
| 216 | A | B | A | A | B | A |
| 218 | A | A | A | A | A | A |
| 219 | A | A | A | A | A | A |
| 220 | A | A | A | A | A | A |
| 221 | A | A | A | A | A | A |
| 222 | A | A | A | A | A | A |
| 223 | A | A | A | A | A | B |
| 224 | A | A | A | A | A | A |
| 225 | B | B | A | A | A | B |
| 226 | B | B | A | A | A | B |
| 227 | A | A | A | A | A | A |
| 228 | A | A | A | A | A | A |
| 229 | A | B | A | A | A | B |
| 230 | A | B | A | A | B | B |
| 231 | A | B | A | A | A | A |
| 232 | A | B | A | A | A | A |
| 233 | A | A | A | A | A | A |
| 234 | A | A | A | A | A | A |
| 235 | A | A | A | A | A | A |
| 237 | A | B | A | A | A | A |
| 238 | A | A | A | A | A | A |
| 239 | B | B | B | B | B | B |
| 240 | A | A | A | A | A | A |
| 241 | A | B | A | A | B | B |
| 243 | A | A | A | A | A | A |
| 244 | A | A | A | A | A | A |
| 245 | A | A | A | A | A | A |
| 246 | A | B | A | A | B | A |
| 247 | A | A | A | A | A | B |
| 248 | A | A | A | A | A | A |
| 249 | A | A | A | A | A | A |
| 250 | A | A | A | A | A | A |
| 251 | A | B | A | A | A | B |
| 252 | A | A | A | A | A | A |
| 253 | A | A | A | A | A | A |
| 254 | A | A | A | A | A | A |
| 255 | A | A | A | A | A | A |
| 256 | A | A | A | A | A | A |
| 257 | A | A | A | A | A | A |
| 258 | A | A | A | A | A | B |
| 259 | A | B | A | A | B | A |
| 260 | A | B | A | B | B | B |
| 261 | A | B | A | B | B | B |
| 262 | A | B | A | B | B | A |
| 263 | A | A | A | B | B | A |
| 264 | B | B | A | B | A | A |
| 265 | B | A | A | A | A | A |
| 266 | A | B | A | A | B | A |
| 267 | A | A | A | A | A | A |
| 268 | A | B | A | A | A | A |
| 269 | A | A | A | A | A | A |
| 270 | A | A | A | A | A | A |
| 271 | A | A | A | A | A | A |
| 272 | A | A | A | A | A | A |
| 273 | A | A | A | A | A | A |
| 274 | A | B | A | A | A | B |
| 275 | A | A | A | A | A | A |
| 276 | A | A | A | A | A | A |
| 277 | A | A | A | A | A | A |
| 278 | A | A | A | A | A | A |
| 279 | A | A | A | A | A | A |
| 280 | A | A | A | B | A | A |
| 281 | A | A | A | A | A | A |
| 282 | A | B | A | A | B | A |
| 283 | A | A | A | A | A | A |
| 284 | A | B | A | B | B | A |
| 285 | A | B | A | B | B | A |
| 286 | A | A | A | A | A | A |
| 287 | A | A | A | A | A | A |
| 288 | A | A | A | A | A | A |
| 289 | A | A | A | A | A | A |
| 290 | A | B | A | A | A | A |
| 291 | B | B | A | B | A | A |
| 292 | A | A | A | A | A | A |
| 293 | A | A | A | A | A | A |
| 294 | A | B | A | B | A | A |
| 295 | A | A | A | A | A | A |
| 296 | A | A | A | A | A | A |
| 297 | A | A | A | A | A | A |
| 298 | A | A | A | A | A | A |
| 299 | A | A | A | A | A | A |
| 300 | A | A | A | A | A | A |
| 301 | B | A | A | B | A | A |
| 302 | A | A | A | A | A | A |
| 303 | A | A | A | A | A | A |
| 304 | A | A | A | A | A | A |
| 305 | A | A | A | A | A | A |
| 306 | A | B | A | A | A | A |
| 307 | A | A | A | A | A | A |
| 308 | A | A | A | B | A | A |
| 309 | A | A | A | A | A | A |
| 310 | B | B | A | A | B | B |
| 311 | B | B | A | B | A | A |
| 312 | A | B | A | A | A | A |
| 313 | A | A | A | A | A | A |
| 314 | A | A | A | A | A | A |
| 315 | A | A | A | A | A | A |
| 316 | A | A | A | A | A | A |
| 317 | A | A | A | A | A | A |
| 318 | A | A | A | A | A | A |
| 319 | A | A | A | A | A | A |
| 320 | A | A | A | A | A | A |
| 321 | A | A | A | A | A | A |
| 322 | A | A | A | A | A | A |
| 323 | A | A | A | A | A | A |
| 324 | A | B | A | A | A | B |
| 325 | B | A | A | A | A | A |
| 326 | A | A | A | A | A | A |
| 327 | A | B | A | B | B | B |
| 328 | A | A | A | A | A | A |
| 329 | A | A | A | B | A | A |
| 330 | A | A | A | A | A | A |
| 331 | A | A | A | A | B | A |
| 332 | A | A | A | A | A | A |
| 333 | A | A | A | A | A | A |
| 334 | A | A | A | A | B | A |
| 335 | A | B | A | B | A | A |
| 336 | A | A | A | A | A | A |
| 337 | A | A | A | A | B | A |
| 338 | A | A | A | A | B | A |
| 339 | A | B | A | A | B | A |
| 340 | A | A | A | A | A | A |
| 341 | A | A | A | A | A | A |
| 342 | A | A | A | A | A | A |
| 343 | A | A | A | A | A | A |
| 344 | A | A | A | A | A | A |
| 345 | A | A | A | A | A | A |
| 346 | A | A | A | A | A | A |
| 347 | A | A | A | A | A | A |
| 348 | A | A | A | A | A | A |
| 349 | A | A | A | A | B | A |
| 350 | A | A | A | A | A | A |
| 351 | A | A | A | A | A | A |
| 352 | A | B | A | A | B | B |
| 353 | A | A | A | A | B | A |
| 354 | A | A | A | A | A | A |
| 355 | A | A | A | A | A | A |
| 356 | A | A | A | A | B | A |
| 357 | A | A | A | A | A | A |
| 358 | A | A | A | A | A | A |
| 359 | A | A | A | A | A | A |
| 360 | A | A | A | A | A | A |
| 361 | A | A | A | A | B | A |
| 362 | A | A | A | A | A | A |
| 363 | A | A | A | A | B | A |
| 364 | A | A | A | A | B | A |
| 365 | A | B | A | B | B | A |
| 366 | A | A | A | A | A | A |
| 367 | A | B | A | A | B | A |
| 368 | A | A | A | A | A | A |
| 369 | A | A | A | A | A | A |
| 370 | A | A | A | A | B | A |
| 371 | A | A | A | A | A | A |
| 372 | A | A | A | A | A | A |
| 373 | A | A | A | A | A | A |
| 374 | A | A | B | A | A | A |
| 375 | A | B | A | A | B | A |
| 376 | A | B | A | A | B | A |
| 377 | A | B | A | A | A | A |
| 378 | A | B | A | A | A | A |
| 379 | A | A | A | A | B | A |
| 380 | A | A | A | A | A | A |
| 381 | A | A | A | A | A | A |
| 382 | A | B | A | A | A | B |
| 383 | A | A | A | A | A | A |
| 384 | A | B | A | B | A | A |
| 385 | A | A | A | A | A | A |
| 386 | A | A | A | A | A | A |
| 387 | A | A | A | A | A | A |
| 388 | A | A | A | B | A | A |
| 389 | A | A | A | A | A | A |
| 390 | A | A | A | A | A | A |
| 391 | A | A | A | A | A | A |
| 392 | A | A | A | A | A | A |
| 393 | A | B | A | A | A | A |
| 394 | B | B | A | A | A | A |
| 395 | A | A | A | A | A | B |
| 396 | A | A | A | A | A | B |
| 397 | A | A | A | A | A | A |
| 398 | A | A | A | B | A | A |
| 399 | A | A | A | A | A | A |
| 400 | A | A | A | A | A | A |
| 401 | A | B | A | B | A | A |
| 402 | A | A | A | A | A | A |
| 403 | A | A | A | A | A | A |
| 404 | A | A | A | A | A | A |
| 405 | A | A | A | A | A | A |
| 406 | A | A | A | A | A | A |
| 407 | A | A | A | A | A | A |
| 408 | A | B | A | A | A | A |
| 409 | A | A | A | A | B | A |
| 410 | A | A | A | B | A | A |
| 411 | A | A | A | A | A | A |
| 412 | A | A | A | B | B | A |
| 413 | A | B | A | B | B | A |
| 414 | A | A | A | A | A | A |
| 415 | A | A | A | A | A | A |
| 416 | A | A | A | A | A | A |
| 417 | A | A | A | B | B | A |
| 418 | A | A | A | A | B | A |
| 419 | A | A | A | A | A | A |
| 420 | A | A | A | A | A | A |
| 421 | A | A | A | A | A | A |
| 422 | A | A | A | A | A | A |
| 423 | A | A | A | A | A | A |
| 424 | A | A | A | A | A | A |
| 425 | A | B | A | B | B | A |
| 426 | A | B | A | B | B | A |
| 427 | A | A | A | B | B | A |
| 428 | A | A | A | A | A | A |
| 429 | A | A | A | A | A | A |
| 430 | A | A | A | A | A | A |
| 431 | A | A | A | B | B | A |
| 432 | A | A | A | A | A | A |
| 433 | A | A | A | A | A | A |
| 434 | A | A | A | A | B | A |
| 435 | A | A | A | A | A | A |
| 436 | A | A | A | A | A | A |
| 437 | A | A | A | A | A | A |
| 438 | A | A | A | A | A | A |
| 439 | A | A | A | A | A | A |
| 440 | A | A | A | A | A | A |
| 441 | A | A | A | A | A | A |
| 442 | A | A | A | A | A | A |
| 443 | A | A | A | A | A | A |
| 444 | A | A | A | B | A | A |
| 445 | A | A | A | A | A | A |
| 446 | A | A | A | A | A | A |
| 447 | A | A | A | B | B | A |
| 448 | A | A | A | A | A | A |
| 449 | A | A | A | A | A | A |
| 450 | A | A | A | A | A | A |
| 451 | A | A | A | A | A | A |
| 452 | A | A | A | A | B | A |
| 453 | A | A | A | A | A | A |
| 454 | A | A | A | A | A | A |
| 455 | A | A | A | A | A | A |
| 456 | A | A | A | B | B | A |
| 457 | A | A | A | A | A | A |
| 458 | A | A | A | A | A | A |
| 459 | A | A | A | A | B | A |
| 460 | A | A | A | A | A | A |
| 461 | A | A | A | A | A | A |
| 462 | A | A | B | A | B | A |
| 463 | A | B | A | A | B | A |
| 464 | A | B | A | A | B | B |
| 465 | B | B | A | A | B | B |
| 466 | A | B | A | A | A | A |
| 467 | A | A | A | A | A | A |
| 468 | A | B | B | B | B | B |
| 469 | A | A | A | A | A | A |
| 470 | A | B | B | B | B | B |
| 471 | A | A | A | A | A | A |
| 472 | A | A | A | A | B | A |
| 473 | A | A | A | A | A | A |
| 474 | A | A | A | A | A | A |
| 475 | A | A | A | A | A | A |
| 476 | A | A | A | A | A | A |
| 477 | A | A | A | A | A | A |
| 478 | A | A | A | A | A | A |
| 479 | A | A | A | A | A | A |
| 480 | A | A | A | A | A | A |
| 481 | A | A | A | A | A | A |
| 482 | A | B | A | B | B | A |
| 483 | A | A | A | B | A | A |
| 484 | A | A | A | A | A | A |
| 485 | A | A | A | A | A | A |
| 486 | A | A | A | A | A | A |
| 487 | A | A | A | A | A | B |
| 488 | A | A | A | A | A | A |
| 489 | A | A | A | A | A | A |
| 490 | A | A | A | A | A | A |
| 491 | A | B | A | A | B | A |
| 492 | A | A | A | A | B | A |
| 493 | A | A | A | A | A | A |
| 494 | A | A | A | A | B | A |
| 495 | A | A | A | A | A | A |
| 496 | A | A | A | a | A | A |
| 497 | A | A | A | A | A | A |
| 498 | A | A | A | A | B | A |
| 499 | A | A | A | A | B | A |
| 500 | A | A | A | A | B | A |
| 501 | A | A | A | A | B | A |
| 502 | A | A | A | A | B | A |
| 503 | A | A | A | A | B | A |
| 504 | A | A | A | A | A | A |
| 505 | A | A | A | A | B | A |
| 506 | A | A | A | A | A | A |
| 507 | A | A | A | A | B | A |
| 508 | A | A | A | A | A | A |
| 509 | A | A | A | A | A | A |
| 510 | A | A | A | A | A | A |
| 511 | A | A | A | A | A | A |
| 512 | A | A | A | A | A | A |
| 513 | A | A | A | A | A | A |
| 514 | A | A | A | A | A | A |
| 515 | A | A | A | A | A | A |
| 516 | A | A | A | A | A | A |
| 517 | A | A | A | A | A | A |
| 518 | A | A | A | A | A | A |
| 519 | A | A | A | A | A | A |
| 520 | A | A | A | A | A | A |
| 521 | A | A | A | A | A | A |
| 522 | A | A | A | A | A | A |
| 523 | A | A | A | A | A | A |
| 524 | A | A | A | A | B | A |
| 525 | A | A | A | A | A | A |
| 526 | A | A | A | A | A | A |
| 527 | A | A | A | A | A | A |
| 528 | A | A | A | A | A | A |
| 529 | A | A | A | A | A | A |
| 530 | A | A | A | A | A | A |
| 532 | A | A | A | A | A | A |
| 533 | A | A | A | A | A | A |
| 534 | A | A | A | A | A | A |
| 535 | A | A | A | A | B | A |
| 536 | A | A | A | A | A | A |
| 538 | A | A | A | A | A | A |
| 539 | A | A | A | A | A | A |
| 540 | A | A | A | A | B | A |
| 541 | A | A | A | A | B | A |
| 542 | A | A | A | A | B | A |
| 543 | A | A | A | A | A | A |
| 544 | A | A | A | A | A | A |
| 545 | A | A | A | A | B | A |
| 546 | A | A | A | A | A | A |
| 547 | A | A | A | A | A | A |
| 548 | A | B | A | A | A | A |
| 549 | A | B | A | A | B | A |
| 550 | A | B | A | A | B | A |
| 551 | A | B | A | A | A | A |
| 552 | A | A | A | B | B | A |
| 553 | A | A | A | A | A | A |
| 554 | A | A | A | B | B | A |
| 555 | A | A | A | A | B | A |
| 556 | A | A | A | A | A | A |
| 557 | A | A | A | A | A | A |
| 558 | A | A | A | A | A | A |
| 559 | A | A | A | A | A | A |
| 560 | A | A | A | A | A | A |
| 561 | A | A | A | A | A | A |
| 562 | A | A | A | A | A | A |
| 563 | A | A | A | A | A | A |
| 564 | A | A | A | A | A | A |
| 565 | A | A | A | A | A | A |
| 566 | A | A | A | A | A | A |
| 569 | A | A | A | A | A | A |
| 570 | A | B | A | A | B | A |
| 571 | A | A | A | A | A | A |
| 572 | A | A | A | A | A | A |
| 573 | A | B | A | A | A | A |
| 574 | A | A | A | A | A | A |
| 575 | A | B | A | A | A | B |
| 577 | A | A | A | A | A | A |
| 579 | A | A | A | A | A | A |
| 580 | A | A | A | A | A | A |
| 581 | A | A | A | A | A | A |
| 582 | A | A | A | B | A | A |
| 583 | A | A | A | A | A | A |
| 584 | A | A | A | A | A | A |
| 585 | A | A | A | A | A | A |
| 586 | A | A | A | A | A | A |
| 587 | A | A | A | A | A | A |
| 588 | A | A | A | A | A | A |
| 589 | A | B | A | A | B | A |
| 590 | A | B | A | A | B | A |
| 591 | A | A | A | A | A | A |
| 592 | A | A | A | A | A | A |
| 593 | A | A | A | A | A | A |
| 594 | A | A | A | A | A | A |
| 595 | A | A | A | A | A | A |
| 596 | A | A | A | A | A | A |
| 597 | A | A | A | A | A | A |
| 598 | A | A | A | A | A | A |
| 599 | A | A | A | A | A | A |
| 600 | A | A | A | A | A | A |
| 601 | A | A | A | A | A | A |
| 602 | A | A | A | A | A | A |
| 603 | A | A | A | A | A | A |
| 604 | A | A | A | A | A | A |
| 605 | A | A | A | A | A | A |
| 606 | A | A | A | A | A | A |
| 609 | A | A | A | A | A | A |
| 610 | A | A | A | A | A | A |
| 611 | A | A | A | A | A | A |
| 612 | A | A | A | A | A | A |
| 613 | A | A | A | A | A | A |
| 614 | A | A | A | A | A | A |
| 615 | A | A | A | A | A | A |
| 618 | A | B | A | B | A | A |
| 619 | A | A | A | A | A | A |
| 620 | A | A | A | A | A | A |
| 621 | B | B | A | B | B | B |
| 622 | A | A | A | A | A | A |
| 623 | A | A | A | A | A | A |
| 624 | A | A | A | A | A | A |
| 625 | A | A | A | A | A | A |
| 626 | A | A | A | A | A | A |
| 627 | A | A | A | A | A | A |
| 628 | A | A | A | A | A | A |
| 629 | A | A | A | A | A | A |
| 630 | A | A | A | A | A | A |
| 631 | A | B | A | A | A | B |
| 632 | A | A | A | A | A | A |
| 633 | A | A | A | A | A | A |
| 634 | A | B | A | A | A | A |
| 635 | B | B | A | B | B | A |
| 636 | A | A | A | B | B | A |
| 638 | A | A | A | A | A | A |
| 640 | A | A | A | A | A | A |
| 642 | A | A | A | A | A | A |
| 643 | A | A | A | A | A | A |
| 644 | A | A | A | A | A | A |
| 645 | A | B | A | A | A | A |
| 646 | A | A | A | A | A | A |
| 647 | A | A | A | A | A | A |
| 648 | A | A | A | A | A | A |
| 649 | A | A | A | A | A | A |
| 650 | A | B | A | A | A | B |
| 654 | A | A | A | B | A | A |
| 655 | A | B | A | A | A | A |
| 656 | A | A | A | A | A | A |
| 657 | A | A | A | A | B | A |

### Example A-3: Preclinical Activity and Differentiation of A Compound of Formula (I)

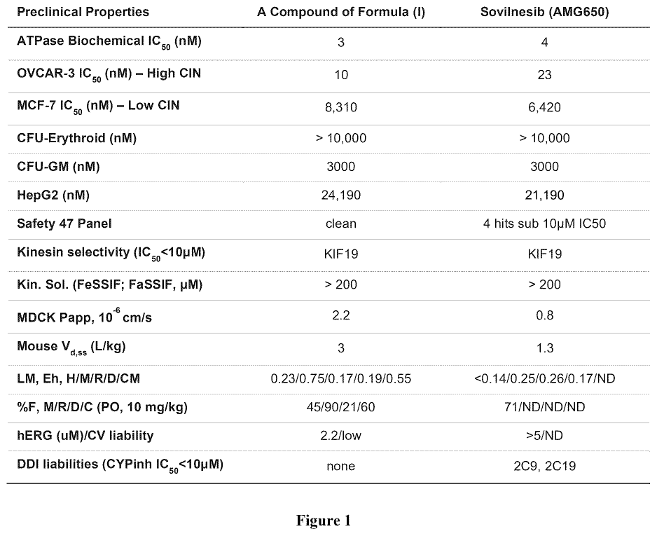

A selected Compound of Formula (I) was compared to sovilnesib (also known as AMG650), a clinical stage KIF18A inhibitor compound under investigation for treatment of cancer, and demonstrated favorable preclinical results. Results from ADME-PK profiling demonstrate that A Compound of Formula (I) is a differentiated, highly potent KIF18A inhibitor with favorable in vitro activity and ADME-PK profile (FIG. 1).

### Methods:

OVCAR-3 and MCF-7 IC₅₀ method—Cell Growth Inhibition assay:

### Cell Lines:

| Name | Vendor | Catalog # | Culture Media |
|---|---|---|---|
| OVCAR-3 | ATCC | HTB-161 | RPMI-1640 + 10% FBS |
| MCF-7 | ATCC | 4637 | RPMI-1640 + 10% FBS |

ATCC = American Type Culture Collection;
FBS = fetal bovine serum;
RPMI = Roswell Park Memorial Institute medium

### Materials:

| Name | Vendor | Catalog # |
|---|---|---|
| RPMI 1640 Medium, 500 mL | ATCC | 30-2001 |
| FBS, HI, certified, | Gibco | A38400-01 |
| One Shot, 50 mL | | |
| Antibiotic-Antimycotic | Gibco | 15240-062 |
| (100X), 100 mL | | |
| PBS, pH 7.4 | Gibco | 10010-023 |
| TrypLE Express | Gibco | 12604013 |
| Enzyme (1X), | | |
| no phenol red | | |
| ViaStain AOPI | Nexcelom | CS2-0106-5mL |
| Staining Solution | Bioscience | |
| 384-well Flat Clear | Corning | 3765 |
| Bottom White Polysterene | | |
| TC-treated Microplates | | |
| CellTiter-Glo 2.0 | Promega | G9243 |
| Assay, 500 mL | | |
| Easy Snap Dispensing | Integra | 5101 |
| Cassette, 5-9,999 uL | | |

A Compound of Formula (I) was dispensed using Echo 655 (Beckman Coulter, Indianapolis IN, USA) in 384-well clear bottom white wall tissue culture (TC) plates, in a ½ log dilution series starting 10000 nM top dose, with 10 doses. Cells were seeded at 500 cells/well for a total volume of 30 uL using the WELLJET dispenser (Integra Biosciences Corp. [New Hampshire, USA]). The cells were allowed to incubate for 3 days in a cell culture incubator at 37° C. and 5% C₀₂. On day 3, 30 μL of CellTiter-Glo 2.0 was added to each well and the luminescent signal was determined using CLARIOstar Plus (BMG Labtech [Ortenberg, Germany]).

Cell growth inhibition was calculated using the following formula:

$$\% Inhibition = 100 \times ( 1 - \frac{{Luminescence}_{sample}}{{Luminescence}_{control}}$$

IC₅₀ values were determined with Prism 9 (GraphPad Software [Boston, MA, USA]) with top and bottom of the curves set at 70% and 0% respectively.

### CFU-Erythroid and CFU-GM Method—In Vitro Evaluation of Test Compounds on Human Erythroid and Myeloid Progenitors Using Colony Forming Cell Assays:

The effects of compounds were tested on human erythroid and myeloid progenitors in a cell colony forming cell assay, performed at Discovery Life Sciences Seattle (formerly ReachBio LLC). Normal human bone marrow light density cells were derived from normal bone marrow (Lonza, Maryland) and stored at −152° C. until needed. On the day of experiment, bone marrow cells were quickly thawed and diluted in Iscove's modified Dulbecco's medium containing 10% fetal bovine serum (IMDM +10% FBS) and washed by centrifugation at approximately 1200 rpm for 10 minutes at room temperature. Cell pellets were resuspended in IMDM+10% FBS upon centrifugation, and cells were plated at 5.0×10⁴ cells per culture after counting and viability check. Test compounds in 10 mM DMSO stocks were serial diluted to working concentrations at 1000× of the desired test concentrations. Final test concentrations (10, 3, 1, 0.1, 0.01, and 0.001 μM) were achieved by adding to the methylcellulose-based medium at 1:1000 (v/v). After 14-day incubation, human myeloid (CFU-GM) and erythroid (CFU-erythroid) colonies were assessed based on size and morphology and scored by trained personnel. The mean±1 standard deviation of three replicate cultures was calculated for progenitors of various lineages. Two-tailed standard t-tests were performed to assess if there was a difference in the number of colonies generated between solvent control and treated cultures. Dose response curve for each compound was generated by plotting the percentage of control colony growth as a function of logarithm of the compound concentrations using Graphpad Prism 9 (GraphPad Software [Boston, MA, USA]) and concentration at 50% inhibition (IC₅₀) was reported.

### Cytotoxicity Screening in HepG2 Cells:

HepG2 cell culture medium consisting of Dulbecco's Modified Eagle's Medium (DMEM) supplemented with 10% FBS, 1 xpenicillin-streptomycin mixture, 1× non-essential amino acids (NEAA) and 1% Hepes was prepared. The cultivated cells in T-75 flasks was rinsed with 5 mL PBS, aspirated off, added 3 mL trypsin/EDTA solution, and incubated at 37° C. for approximately 2 minutes or until the cells detached and floated. Trypsin/EDTA was inactived by adding cell culture medium containing FBS. The cell suspension was removed to a conical tube and pellet cells by centrifugation at 150×g for 5 minutes. The supernatant was aspirated carefully and HepG2 cells were re-suspended in cell culture medium at a density of 150000 cells/mL. 20 μL of above cell suspension was added to each well of 384-well plate. The plates were incubated overnight for the toxicity assay, 37° C., 5% CO2 atmosphere. Test compound was 3-fold serial diluted for 8 doses and started from 30 mM of stock solution. Control compound was 3-fold serial diluted for 8 doses and started from 30 mM of stock solution. The plate was removed from the incubator and directly added 100 nL of the positive control and test article solutions, each in triplicate. The plate(s) was returned to the incubator for 48 hours in a humidified, 37° C., 5% CO2. After the incubation the plates were removed from incubator. Pre-mixed Cell Titer-Glo reagent (20 μL per well) was added directly into the 384-well plates. The plates were shaken for 5 min on a plate shaker. The plates were incubated at room temperature for 10 minutes. After 10 minutes, the luminescence was recorded on a plate reader. Tamoxifen was used as reference control. % Vehicle Control of test compound was calculated using the following equation:

$$\% Vehicle control = [ ( Read Compound - Read Blank ) / ( Read Vehicle - Read Blank ) ] * 100 \%$$

% Vehicle Control was plotted against the concentration of test compound and the data was fitted to a sigmoidal dose-response curve with a variable slope using GraphPad Prism8.0.2. The IC₅₀ was calculated from the curve as the following equation:

$$Y = Bottom + ( Top - Bottom ) / ( 1 + 10 \hat{} ( ( Log IC 50 - X ) * HillSlope ) )$$

SAFETYScan E/IC50 ELECT for 78 Targets was Conducted at Eurofins. Assay Formats:

GPCR cAMP Assay, Gs and Gi Agonist Format, Gs and Gi Antagonist Format; GPCR Calcium Assay; Nuclear Hormone Receptor Assay; Neurotransmitter Transporter Uptake Assay; Ion Channel Assays—hERG Potassium Assay; Ion Channel Assays—KvLQT1/minK Potassium Assay; Ion Channel Assays—Calcium Assay; Ion Channel Assays—Membrane Potential Assay; Ion Channel Assays —Membrane Potential Assay; Enzymatic Assays, KINOMEscan® Assay. Target list and assay details are provided at Eurofins website: www.eurofinsdiscovery.com

### Kinesin Selectivity—ADP-Glo Kinesin Panel:

ATPase Profiling of KIF10 (CENP-E), KIF11 (Eg5), KIF18A, KIF18B, KIF19, KIF20A (MKLP2), KIF22, KIF23 (MKLP1), KIF2C (MCAK), KIF3C, KIF4 (Chromokinesin), KIF5B (Kinesin heavy chain), KIFC, KIFC3 was conducted at ReactionBiology. Compounds were received in a plate at a 10 mM stock concentration in DMSO. Compounds were tested in 10-dose IC50 singlet with 3-fold serial dilution starting at 50 or 10 μM. Control compounds (PF-2771, ARQ 621, Sovilnesib, Paprotrain, Ispinesib (SB-715992), AZ82) were tested in 10-close IC50 with 3-fold serial dilution starting at 100, 10 or 1 μM. Reactions were carried out at 10 μM ATP. ADP-Glo assay format was used. Curve fits were performed where the enzyme activities at the highest concentration of compounds were less than 65%. Kinetic solubility determination FeSSIF and FaSSIF:

Preparation of FaSSIF. Buffer A: Dissolve 1.392 g of NaOH, 2.220 g of Maleic acid and 4.010 g of NaCl into about 900 mL ultrapure water and adjust the pH of the solution to 6.5 with 1 M NaOH. Then dilute the solution with ultrapure water to 1000 mL at room temperature. Add 1.790 g of FaSSIF-V2 powder to about 500 mL of buffer A. Stir until powder is completely dissolved. Then dilute the solution with buffer A to 1000 mL at room temperature. Use within 48 hours at room temperature and 24 hours at 37° C. Preparation of FeSSIF. Buffer A: Dissolve 3.27 g of NaOH, 6.39 g of Maleic acid and 7.33 g of NaCl into about 900 mL ultrapure water and adjust the pH of the solution to 5.8 with 1 M NaOH. Then dilute the solution with ultrapure water to 1000 mL at room temperature. Add 9.760 g of FeSSIF—V2 powder to about 500 mL of buffer A. Stir until powder is completely dissolved. Then dilute the solution with buffer A to 1000 mL at room temperature. Use within 48 hours at room temperature and 24 hours at 37° C. The stock solution of test compounds was prepared in DMSO at the concentration of 10 mM. The stock solution of positive control compound was prepared in DMSO at the concentration of 30 mM. Diclofenac was used as positive control in the assay. 30 μL stock solution of each compound was placed in order into their proper 96-well rack, followed by adding 970 μL of FeSSIF or SGF into each vial of the cap-less Solubility Sample plate. This study was performed in duplicate. One stir stick was added to each vial and then vials were sealed using a molded PTDE/SIL 96-Well Plate Cover. The Solubility Sample plate was transferred to the Thermomixer Comfort plate shaker and incubated at RT for 2 hours or 6 hours with shaking at 1100 rpm. After 2 hours or 6 hours incubation, stir sticks were removed using a big magnet and all samples from the Solubility Sample plate were transferred into the filter plate. All the samples were filtered by using the Vacuum Manifold. An aliquot of 10 μL of the filtered samples were diluted with 980 μL of methanol and 10 μL of DMSO. The dilution factor might be changed according to the solubility value and the LC/MS signal response. Preparation of 3 μM Standards (STD): DMSO stock solution at 10 mM was diluted with DMSO to 300 μM. An aliquot of 10 μL was transferred to a new 96-well plate followed by adding of 980 μL of methanol and 10 μL of FeSSIF or SGF to obtain 3 μM STDs. The dilution factor might be changed according to the LC/MS signal response. Samples were analyzed by LC-MS/MS. The solution filtered was analyzed and quantified against a standard of known concentration in DMSO using LC coupled with Mass spectral peak identification and quantitation. The solubility values of the test compounds were calculated as follows (DF is dilution factor):

$$[ Sample ] = \frac{{AREA}_{Sample} \times INJ {VOL}_{Std} \times {DF}_{Sample} \times [ STD ]}{{AREA}_{Std} \times INJ {VOL}_{Sample}}$$

### Permeability Determination in MDCK Cells:

50 μL and 25 mL of cell culture medium were added to each well of the Transwell insert and reservoir, respectively. And then the HTS transwell plates were incubated at 37° C., 5% CO₂ for 1 hour before cell seeding. MDCK cells were diluted to 1.56×106 cells/mL with culture medium and 50 μL of cell suspension were dispensed into the filter well of the 96-well HTS Transwell plate. Cells were cultivated for 3-8 days in a cell culture incubator at 37° C., 5% CO2, 95% relative humidity. Cell culture medium was replaced every other day, beginning no later than 24 hours after initial plating. 10 mM stock solutions of test compounds were prepared in DMSO. The stock solutions of positive controls were prepared in DMSO at the concentration of 10 mM. Metoprolol, Digoxin and imatinib were used as control compounds in this assay. Assessment of Cell Monolayer Integrity: Medium was removed from the reservoir and each Transwell insert and replaced with prewarmed fresh culture medium. Transepithelial electrical resistance (TEER) across the monolayer was measured using Millicell Epithelial Volt-Ohm measuring system (Millipore, USA). The Plate was returned to the incubator once the measurement was done. The TEER value was calucated according to the following equation:

TEER measurement (ohms)×Area of membrane (cm2)=TEER value (ohm·cm2)

TEER value should be greater than 42 ohm·cm2, which indicates the well-qualified MDCK monolayer.

The MDCK plate was removed from the incubator and washed twice with pre-warmed HBSS (10 mM HEPES, pH 7.4), and then incubated at 37° C. for 30 minutes. The stock solutions of test compounds were diluted in DMSO to get 0.2 mM solutions and then diluted with HBSS (10 mM HEPES and 2% BSA, pH 7.4) to get 1 μM working solutions. The stock solutions of controls were diluted in DMSO to get 0.2 mM solutions and then diluted with HBSS (10 mM HEPES, pH 7.4) to get 1 μM working solutions. The final concentration of DMSO in the incubation system was 0.5%. To determine the rate of drug transport in the apical to basolateral direction. 75 μL of 1 μM working solution of test compound was added to the Transwell insert (apical compartment) and the wells in the receiver plate (basolateral compartment) were filled with 235 μL of HBSS (10 mM HEPES and 2% BSA, pH 7.4). 75 μL of 1 μM working solution of control compound was added to the Transwell insert (apical compartment) and the wells in the receiver plate (basolateral compartment) were filled with 235 μL of HBSS (10 mM HEPES, pH 7.4). The assay was performed in duplicate. To determine the rate of drug transport in the basolateral to apical direction. 235 μL of 1 μM working solution of test compound was to the receiver plate wells (basolateral compartment) and then the Transwell inserts (apical compartment) were filled with 75 μL of HBSS (10 mM HEPES and 2% BSA, pH 7.4). 235 μL of 1 μM working solution of control compound was added to the receiver plate wells (basolateral compartment) and the Transwell inserts (apical compartment) were filled with 75 μL of HBSS (10 mM HEPES, pH 7.4). Time 0 samples were prepared by transferring 50 μL of 1 μM working solution to wells of the 96-deepwell plate, followed by the addition of 200 μL cold methanol containing appropriate internal standards (100 nM Ketoprofen, 200 nM labetalol, 200 nM caffeine and 200 nM diclofenac). The plates were incuabted at 37° C. for 2 hours. At the end of the incubation, 50 μL samples from donor sides (apical compartment for Ap→B1 flux, and basolateral compartment for B→Ap) and receiver sides (basolateral compartment for Ap→B1 flux, and apical compartment for B1→Ap) were transferred to wells of a new 96-well plate, followed by the addition of 4 volume of cold methanol containing appropriate internal standards (100 nM Ketoprofen, 200 nM labetalol, 200 nM caffeine and 200 nM diclofenac). Samples were vortexed for 5 minutes and then centrifuged at 3,220 g for 40 minutes. An aliquot of 100 μL of the supernatant was mixed with 100 μL of ultra-pure water. The samples were analyzed by LC-MS/MS. To determine the Lucifer Yellow leakage after 2 hour transport period, stock solution of Lucifer yellow was prepared in water and diluted with HBSS (10 mM HEPES, pH 7.4) to reach the final concentration of 100 μM. 100 μL of the Lucifer yellow solution was added to each Transwell insert (apical compartment), followed by filling the wells in the receiver plate (basolateral compartment) with 300 μL of HBSS (10 mM HEPES, pH 7.4). The plates were Incubated at 37° C. for 30 mins. 80 μL samples were removed directly from the apical and basolateral wells (using the basolateral access holes) and transferred to wells of new 96 wells plates. The Lucifer Yellow fluorescence (to monitor monolayer integrity) signal was measured in a fluorescence plate reader at 485 nM excitation and 530 nM emission. The apparent permeability coefficient (Papp), in units of centimeter per second, can be calculated for MDCK drug transport assays using the following equation:

$$Papp = ( VA \times [ drug ] acceptor ) / ( Area \times Time \times [ drug ] initial , donor )$$

Where VA is the volume (in mL) in the acceptor well, Area is the surface area of the membrane (0.143 cm2 for Transwell-96 Well Permeable Supports), and time is the total transport time in seconds.

The recovery can be determined using the following equation:

$$Recovery \% = ( VA \times [ drug ] acceptor + VD \times [ drug ] donor ) / ( VD \times [ drug ] initial , donor )$$

Where VA is the volume (in mL) in the acceptor well (0.235 mL for Ap→B1 flux, and 0.075 mL for B1→Ap), VD is the volume (in mL) in the donor well (0.075 mL for Ap→B1 flux, and 0.235 mL for B1→Ap)

The leakage of Lucifer Yellow, in unit of percentage (%), can be calculated using the following equation:

$$\% LY leakage = 100 \times [ LY ] acceptor \times 0.3 / ( [ LY ] donor \times 0.1 + [ LY ] acceptor \times 0.3 )$$

LY leakage of <1% is acceptable to indicate the well-qualified MDCK monolayer.

### Metabolic Stability in Liver Microsomes For Cyno Monkey:

| | Stock | | | | Final | |
|---|---|---|---|---|---|---|
| Reagent | Concentration | | Volume | | Concentration | |
| Phosphate | 114.94 | mM | 69.6 | μL | 111.11 | mM |
| buffer | | | | | | |
| Ultra-pure H₂O | — | | 0.4 | μL | — | |
| Microsomes | 20 | mg/mL | 2 | μL | 0.556 | mg/mL |

10 mM NADPH was prepared in ultra-pure H₂O. 80 nL of 1 mM test compounds were added to the 72 μL of master solution. The reaction was started with the addition of 8 μL of 10 mM NADPH solution. The final concentration of NADPH solution was 1 mM. The negative control samples were prepared by replacing NADPH solutions with 8 μL of ultra-pure H₂O. The negative control was used to exclude the misleading factor that resulted from instability of chemical itself. Samples with NADPH were prepared in duplicate. Negative controls were prepared in singlet. Verapamil was used as positive control in this study. The final concentration of test compounds or control compound was 1 μM and DMSO concentration in the incubation system was 0.1%. The mixture was pre-warmed at 37° C. for 10 minutes. Aliquots of 10 μL of 4 test compounds (40 μL in total) were taken from the reaction solution into one well of a new plate containing 120 μL of cold acetonitrile with internal standards (1S, 200 nM labetalol, 100 nM tolbutamide and 100 nM ketoprofen). The same operation was done after 15-, 30-, 45- and 60 minutes incubation at 37° C. Samples were centrifuged at 3, 220 g for 45 minutes. Aliquot of 40 μL of the supernatant was mixed with 40 μL of ultra-pure H₂O and then was used for LC-MS/MS analysis. Peak areas were determined from extracted ion chromatograms. The slope value, k, was determined by linear regression of the natural logarithm of the remaining percentage of the parent drug vs. incubation time curve. The in vitro half-life (in vitro t_{1/2}) was determined from the slope value:

$$in vitro {t}_{1 / 2} = - \{ 0.693 / k )$$

Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{int} = \frac{0.693}{{t}_{1 / 2}} \times \frac{volume of incubation ( μ L )}{amount of proteins ( mg )}$$

### Metabolic Stability in Liver Microsomes For Human, Mouse, Rat and Dog:

| | | | |
|---|---|---|---|
| | Stock | | Final |
| Reagent | Concentration | Volume | Concentration |

| | | | | | | |
|---|---|---|---|---|---|---|
| Phosphate | 107.8 | mM | 11.685 | μL | 105 | mM |
| buffer | | | | | | |
| Microsomes | 20 | mg/mL | 0.315 | μL | 0.525 | mg/mL |

200 mM NADPH was prepared in ultra-pure H₂O. 5 nL of 2.5 mM test compounds were added to the 12 μL of master solution (100 mM PBS, 0.5 mg/mL microme). The reaction was started with the addition of 0.6 μL of 20 mM NADPH solution. The final concentration of NADPH solution was 1 mM. The negative control samples were prepared by replacing NADPH solutions with 0.6 μL of ultra-pure H₂O. The negative control was used to exclude the misleading factor that resulted from instability of chemical itself. Samples with NADPH were prepared in duplicate. Negative controls were prepared in singlet. Propranolol and Quinidine were used as positive controls in this study. The final concentration of test compounds or control compound was 1 μM and DMSO concentration in the incubation system was 0.04%. The mixture was pre-warmed at 37° C. for 5 minutes. Reactions were quenched with 12.5 ul of cold acetonitrile with internal standard (1S, 50 nM Verapamil). The same operation was done after 7.5-, 15-, 30-, 45- and 60 minutes incubation at 37° C. Samples were centrifuged at 3, 220 g for 15 minutes. Quenched samples was mixed with 25 μL of ultra-pure H₂O and then was used for LC-MS/MS analysis. Peak areas were determined from extracted ion chromatograms. The slope value, k, was determined by linear regression of the natural logarithm of the remaining percentage of the parent drug vs. incubation time curve. The in vitro half-life (in vitro t_{1/2}) was determined from the slope value: LN(2)/slope. Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{int} = \frac{0.693}{{t}_{1 / 2}} \times \frac{volume of incubation ( μ L )}{amount of proteins ( mg )}$$

hERG Assay ICso Determination on Cloned hERG Potassium Channels (Encoded by the KCNH2 Gene and Expressed in HEK293 Cells):

The in vitro effects of test articles were evaluated at 33° C. using the QPatch II® (Sophion Bioscience A/S, Denmark), an automatic parallel patch clamp system. Test article concentrations were prepared by diluting stock solutions into an appropriate HEPES-buffered physiological saline solution (137 mM NaCl, 4 mM KCl, 1.8 mM CaCl₂, 1 mM MgCl₂, 10 mM HEPES, 10 mM Glucose, pH adjusted to 7.4). HEK293 cells were stably transfected with the appropriate ion channel cDNA(s). Except for cells that have been stored frozen, stable transfectants were maintained in the culture medium with the appropriate selection pressure and antibiotics. Vehicle was applied via the QPatch robot pipetting system to naïve cells for a 5-10 minute exposure interval. After vehicle application, the test article concentrations specified below were applied in at least three (3) minute intervals (n≤3), where n=the number of cells/concentration. Each solution exchange on the QPatch was performed multiple times, which resulted in 100% replacement of the compound in the QPlate. Positive control (Cisapride) was applied in the same manner as the test article (n≥2) to verify sensitivity to ion channel blockade.

In preparation for a recording session, intracellular solution (130 mM K-Asp, 5 mM MgCl₂, 4 mM Tris-ATP, 10 mM HEPES, 5 mM EGTA, pH adjusted to 7.2) was loaded into the intracellular compartments of the QPlate and cell suspension was pipetted into the extracellular compartments. After establishment of a whole-cell configuration, membrane currents were recorded using up to 48 parallel patch clamp amplifiers in the QPatch II© or system.

Valid whole-cell recordings had met the following criteria:
Membrane resistance 1 Gn.
Leak current 10% channel current or subtracted.

The liquid junction potential between the internal pipette solution and the hERG buffer was calculated to be approximately 15.7 mV; voltages listed below was adjusted by this voltage. Cells stably expressing hERG was held at −80 mV; from a holding potential of −80 mV, voltage was stepped to −90 mV for 100 ms and then to −80 mV for 100 ms prior to the depolarizing pulse. The voltage was then stepped to +40 mV for 500 ms followed by a 100 ms ramp to −80 mV (rate of 1.2 V/s). This voltage protocol (˜5 seconds in duration) was repeated at 5 second intervals. Onset and steady state inhibition of hERG potassium current due to test article application was measured during the ramp phase of the voltage stimulus pattern. Each recording ended with a final application of a supramaximal concentration of the reference substance (E-4031, 1 μM), to assess the contribution of endogenous currents. The remaining uninhibited current was subtracted off-line digitally from the data to determine the potency of the test substance for hERG inhibition. This voltage protocol is approximately 5 s in duration, and was repeated every 5 s. The voltage “ramp down” phase is 100 ms in duration, from +40 mV to −80 mV (hence a voltage change of −1.2 V/s).

Series resistance compensation was performed. The cut-off frequency was 2 kHz and sampling frequency was 5 kHz. One to two test article concentrations were applied sequentially (without washout between test article concentrations) in ascending order, to each cell. Peak current was measured during the test ramp. Peak tail current was measured until a new steady state is achieved. Drug effect was monitored until steady state hERG current suppression was obtained. To ensure the baseline recording was stable enough for drug application, cells were presented with this protocol in control solution until hERG current amplitudes for 25 consecutively recorded current traces exhibit 10% difference.

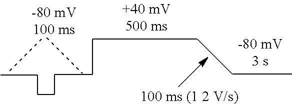

A Compound of Formula (I) was exposed to hERG at 0.3, 1, 3, 5, 10, and 30 μM (n 3). Sovilnesib was exposed to hERG at 3 and 5 μM (n≥3). The duration of exposure to each test article concentration was at least 3 minutes. Steady state is defined by the limiting constant rate of change with time (linear time dependence). The steady state before and after test article application was used to calculate the percentage of current inhibited at each concentration.

If current is blocked ≥50%, estimated IC50 values was calculated using the following:

$$\% Inhibition = \{ 1 - 1 / [ 1 + {( [ Test ] / {IC}_{50} )}^{N} ] \} * 100$$

Where [Test] is the test article concentration, IC₅₀ is the test article concentration at half-maximal inhibition, N is the Hill coefficient, and % Inhibition is the percentage of current inhibited at each test article concentration. Nonlinear least squares fits were solved with the Solver add-in for Excel (Microsoft, WA)

Overestimation of current inhibition, due to rundown, was avoided by a manual adjustment that uses an exponential compensation formula in the QPatch analysis software.

### PK Data Collection Details:

IV (1 mg/kg, 5 mL/kg, vehicle 20% DMSO, 40% PEG400, 40% Water) dosing in female CD-1 mice.
PO (10 mg/mL, 10 mL/kg, vehicle 1% Tween 80, 2% HPMC, 97% water) dosing in female CD-1 mice.
PO (10 mg/mL, 10 mL/kg, vehicle 50% Labrasol, 50% PEG400) dosing in female SD rat.
PO (10 mg/mL, 2 mL/kg, vehicle 1% Tween 80, 2% HPMC, 97% water) dosing in female beagle dog.
PO (10 mg/mL, 2 mL/kg, vehicle 50% Labrasol, 50% PEG400) dosing in male cyno monkey.

### Example A-4: Anti-proliferative Activity of A Compound of Formula (I)

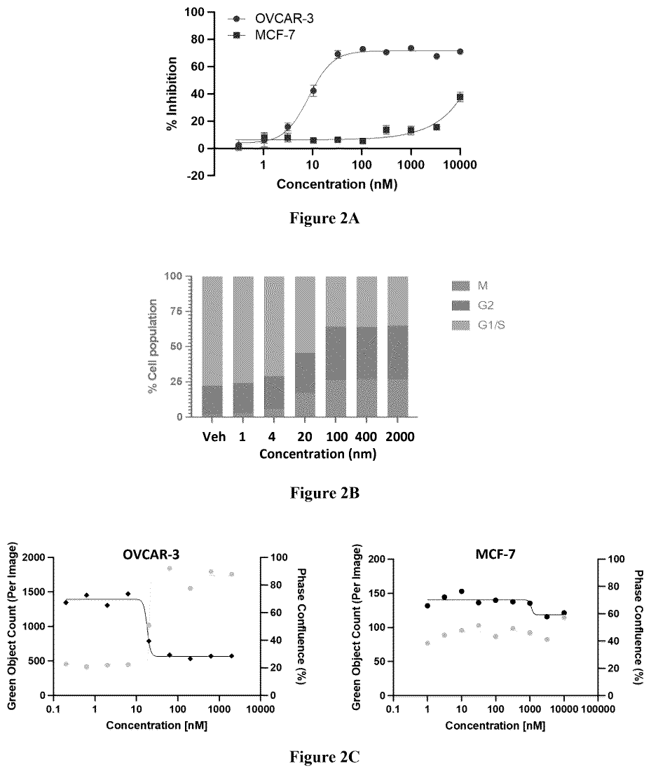

The anti-proliferative activity of A Compound of Formula (I) correlates with mitotic arrest and apoptosis (FIG. 2). FIG. 2A shows a 6-day viability assay with A Compound of Formula (I) dose response curve in OVCAR-3 and MCF-7 cells. OVCAR-3 cells are sensitive to KIF18A inhibition by A Compound of Formula (I) and MCF-7 cells are not. FIG. 2B shows a dose dependent increase in M phase quantified by flow cytometry using phospho-histone H3 (Ser10) Alexa 488 antibody+FxCycle™ Violet stain with A Compound of Formula (I). FIG. 2C shows dose response for 72 hours of OVCAR-3 (left panel) and MCF-7 (right panel) cells treated with A Compound of Formula (I). Incucyte v.2023A was used to quantify cell confluence and cell death (via Caspase-3/7 green apoptosis reagent). Treatment with A Compound of Formula (I) results in increased apoptosis and reduced confluence in OVCAR-3 cells but not MCF-7 cells.

### Methods:

### Cell Growth Inhibition Assay:

### Cell Lines:

| Name | Vendor | Catalog # | Culture Media |
|---|---|---|---|
| OVCAR-3 | ATCC | HTB-161 | RPMI-1640 + 10% FBS |
| MCF-7 | ATCC | 4637 | RPMI-1640 + 10% FBS |

ATCC = American Type Culture Collection;
FBS = fetal bovine serum;
RPMI = Roswell Park Memorial Institute medium

### Materials:

| Name | Vendor | Catalog # |
|---|---|---|
| RPMI 1640 Medium, 500 mL | ATCC | 30-2001 |
| FBS, HI, certified, | Gibco | A38400-01 |
| One Shot, 50 mL | | |
| Antibiotic-Antimycotic | Gibco | 15240-062 |
| (100X), 100 mL | | |
| PBS, pH 7.4 | Gibco | 10010-023 |
| TrypLE Express Enzyme | Gibco | 12604013 |
| (1X), no phenol red | | |
| ViaStain AOPI | Nexcelom | CS2-0106-5mL |
| Staining Solution | Bioscience | |
| 384-well Flat Clear | Corning | 3765 |
| Bottom White Polysterene TC- | | |
| treated Microplates | | |
| CellTiter-Glo | Promega | G9243 |
| 2.0 Assay, 500 mL | | |
| Easy Snap Dispensing | Integra | 5101 |
| Cassette, 5-9,999 uL | | |

A Compound of Formula (I) was dispensed using Echo 655 (Beckman Coulter, Indianapolis IN, USA) in 384-well clear bottom white wall tissue culture (TC) plates, in a ½ log dilution series starting 10000 nM top dose, with 10 doses. Cells were seeded at 500 cells/well for a total volume of 30 uL using the WELLJET dispenser (Integra Biosciences Corp. [New Hampshire, USA]). The cells were allowed to incubate for 3 days in a cell culture incubator at 37° C. and 5% CO2. On day 3, 30 uL of CellTiter-Glo 2.0 was added to each well and the luminescent signal was determined using CLARIOstar Plus (BMG Labtech [Ortenberg, Germany]). Fit curves were plotted with Prism 9 (GraphPad Software [Boston, MA, USA]).

### Mitotic Arrest Assay: Flow Cytometry Assay:

OVCAR-3 cells were seeded in T25 flasks (5 days in culture, density of 500.000 cells/flask) and treated for 24 h with increasing concentrations of A Compound of Formula (I). After 24 h cells were trypsinized and washed with complete RPMI medium (1500 rpm, 5 min). Following centrifugation cells were suspended with calcium free apoptotic-dead cell tag 488 (BioMegis) for 10 minutes at room temperature followed by one wash with 3 mL of staining buffer. The cells were fixed, permeabilized and stained with Alexa 488 Phospho Histone H3 (CST #3465). The cells were stained with FxCycle™ Violet Stain (Invitrogen #F10347) for 30 minutes before analysis followed by Invitrogen provided protocol. Samples were analyzed using a FACSCalibur Flow Cytometer (BD Biosciences) and data processing was done using FlowJo software. Statistical significance was determined for each group relative to the DMSO control by one-way ANOVA at a significance level of 0.05. M-phase analysis (24-h) shows accumulation of M-phase population with increasing concentration of A Compound of Formula (I) (1 nM to 2 μM relative to DMSO vehicle control) in OVCAR-3 cell line.

### Cell Apoptosis Assay Method: Live Cell Imaging:

### Cell Line:

| Name | Vendor | Catalog # |
|---|---|---|
| OVCAR-3 | ATCC | HTB-161 |
| MCF-7 | ATCC | 4637 |

### Materials:

| Name | Vendor | Catalog # |
|---|---|---|
| RPMI 1640 Medium, 500 mL | ATCC | 30-2001 |
| RPMI 1640 Medium, | Gibco | 11835-030 |
| no phenol red | | |
| FBS, HI, certified, | Gibco | A38400-01 |
| One Shot, 50 mL | | |
| Antibiotic-Antimycotic | Gibco | 15240-062 |
| (100X), 100 mL | | |
| PBS, pH 7.4 | Gibco | 10010-023 |
| TrypLE Express Enzyme (1X), | Gibco | 12604013 |
| no phenol red | | |
| ViaStain AOPI | Nexcelom | CS2-0106-5mL |
| Staining Solution | Bioscience | |
| Incucyte Caspase-3/7 | Sartorius | 4440 |
| Green Apoptosis Reagent | | |
| Incucyte Nuclight | Sartorius | 4717 |
| Rapid Red Dye | | |
| Incucyte Imagelock | Sartorius | BA-04855 |
| Plate 96-well, | | |
| TC-treated | | |

### Equipment:

| Name | Vendor | Catalog/Model # |
|---|---|---|
| Forma Series 3 Water | Thermo | 4120 |
| Jacketed CO2 Incubator | Scientific | |
| Cellometer K2 | Nexcelom | K2 SK-150 |
| Image Cytometer | Bioscience | |
| Incucyte S3 | Sartorius | 4637 |

### Software:

| Name | Vendor | Version |
|---|---|---|
| Incucyte 2023A | Sartorius | 2023A Rev2 |
| Prism 9 | GraphPad | GraphPad Prism 9.3.1 |
| | | (350) Macintosh Version |

OVCAR-3 cell line was obtained from American Type Culture Collection (American Type Culture Collection [ATCC]; Manassas, VA, USA) and cultured in Roswell Park Memorial Institute (RPMI) 1640 medium supplemented with 10% fetal bovine serum (FBS). Cells were seeded in a 96-well plate at 10,000 cells/well in 0.1 mL in RPMI 1640, no phenol red medium supplemented with 10% FBS +1× Antibiotic-Antimycotic, excluding edge wells. The edge wells were filled with 0.1 mL of complete culture medium. Cells were allowed to adhere overnight @37° C. in a 5% CO2 incubator. Media was removed from each well and replaced with 0.1 mL of complete culture medium with 5 mM Incucyte Caspase-3/7 Green Apoptosis Reagent (Sartorius [Gottingen, Germany]) and 1:500 Incucyte Nuclight Rapid Red Dye (Sartorius [Gottingen, Germany]). Cells were treated with A Compound of Formula (I) at 0, 0.20, 0.63, 2.0, 6.3, 20.0, 63.3, 2000, 632.5, 200.0, 632.5 and 2000 nM for 72 hours at 37° C., 5% CO2. Images were taken on the Incucyte S3 imaging system (Sartorius [Gottingen, Germany]), using 10× objective, on phase and fluorescent channels, every 4 hours.

MCF-7 cell line was obtained from ATCC (Manassas, VA, USA) and cultured in RPMI 1640 medium supplemented with 10% FBS. Cells were seeded in a 96-well plate at 10,000 cells/well in 0.1 mL in RPMI 1640, no phenol red medium supplemented with 10% FBS+1× Antibiotic-Antimycotic, excluding edge wells. The edge wells were filled with 0.1 mL of complete culture medium. Cells were allowed to adhere overnight @37° C. in a 5% CO₂ incubator. Media was removed from each well and replaced with 0.1 mL of complete culture medium with 5 mM Incucyte Caspase-3/7 Green Apoptosis Reagent (Sartorius [Gottingen, Germany]) and 1:500 Incucyte Nuclight Rapid Red Dye (Sartorius [Gottingen, Germany]). Cells were treated with A Compound of Formula (I) at 0, 0.20, 0.63, 2.0, 6.3, 20.0, 63.3, 2000, 632.5, 200.0, 632.5 and 2000 nM for 72 hours at 37° C., 5% CO2. Images were taken on the Incucyte S3 imaging system (Sartorius [Gottingen, Germany]), using 10× objective, on phase and fluorescent channels, every 4 hours.

Quantification of green fluorescent signal and phase confluence was performed on Incucyte 2023A (Sartorius). Graphical analysis of the test compound was performed with Prism 9 (GraphPad Software [Boston, MA, USA]).

### Example A-5: Anti-proliferative Activity of A Compound of Formula (I) in Breast and Ovarian Cell Lines

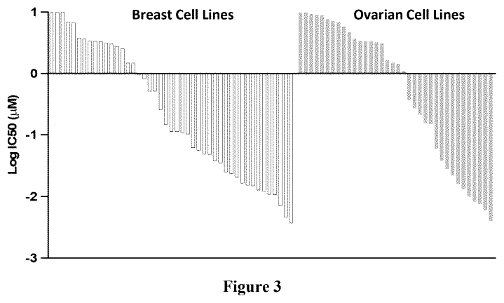

A Compound of Formula (I) results in potent anti proliferative activity in a large panel of breast and ovarian cell lines. FIG. 3 shows IC₅₀s for A Compound of Formula (I) in a broad panel of cell lines, as assessed by 6-day CellTiter-Glo assay.

### Methods:

### Cell Proliferation Measurement Using the CellTiter-Glo Assay (Promega):

Cells were seeded into 384-well plates (#3764, Corning) in 40 μL/well at a standard cell density and culture medium recommended by ATCC or vendors and incubated overnight at 37° C./5% CO₂. Compounds were added to the cells by using a Tecan (EVO200) digital dispenser (Tecan, Switzerland), to achieve final concentrations ranging from 10 μM highest and 0.32 nM lowest for cancer cell lines (3.16-fold, 10-points dilution). DMSO concentration was normalized to 0.1% in all wells by adding the corresponding volume of DMSO with the digital dispenser. Compound effects were assessed after 6 days of incubation at 37° C./5% CO2 by adding 40 μL per well CellTiter-Glo substrate (Promega #G7573). After 30 minutes of incubation at room temperature, the luminescence intensity was measured by Envision. The Inhibition % expressed as the following formula:

$$\% Inhibition = 100 \times ( ReadoutHC - ReadoutSample ) / ( ReadoutHC - ReadoutLC ) .$$

Graphical analysis of the test compound was performed with Prism 9 (GraphPad Software [Boston, MA, USA]).

### Example A-6: Efficacy in OVCAR-3 Xeno2Raft Model

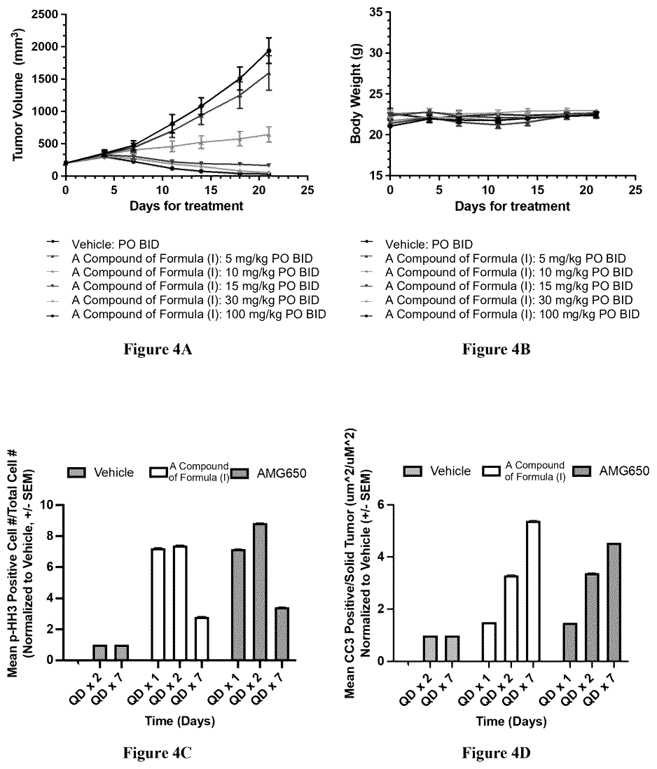

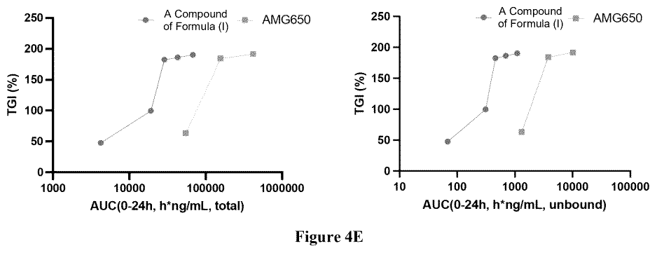

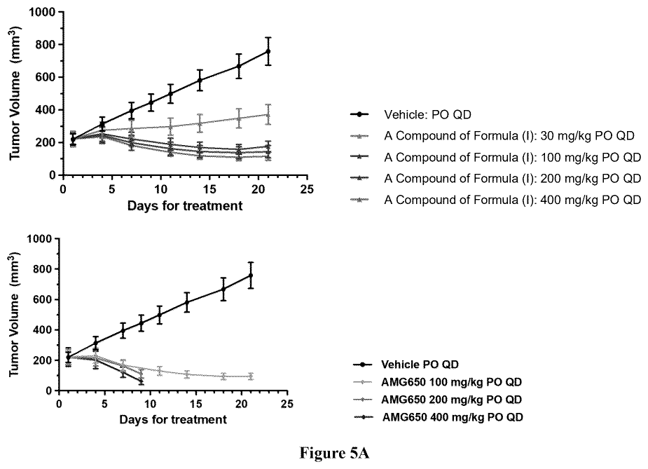

A Compound of Formula (I) demonstrates dose dependent tumor growth inhibition (TGI) and biomarker modulation in OVCAR-3 xenograft model (FIG. 4). Treatment with A Compound of Formula (I) for 21 days achieves dose dependent antitumor activity (FIG. 4A) and no body weight loss (FIG. 4B). Mice bearing OVCAR-3 tumors were treated with A Compound of Formula (I) at 100 mg/kg QD PO×1 day, 2 days or 7 days. Tumors were harvested and stained for phospho-histone H3 (pHH3) (FIG. 4C) or cleaved caspase-3 (CC-3) (FIG. 4D) and images were quantified for positive cells compared to total cells. Treatment with A Compound of Formula (I) leads to mitotic arrest early in the treatment phase followed by apoptosis at the later time points. FIG. 4E shows that A Compound of Formula (I) achieves similar antitumor activity as AMG650, but at ˜10× lower total (left panel) and unbound (right panel) drug exposures. Lower systemic exposure may result in better tolerability and toxicity profile for A Compound of Formula (I).

### Methods:

### Efficacy and Body Weight Evaluation in OVCAR-3 Xenograft Model:

All the procedures related to animal handling, care, and the treatment were performed according to guidelines approved by the Institutional Animal Care and Use Committee (IACUC) of Pharmaron following the guidance of the Association for Assessment and Accreditation of Laboratory Animal Care (AAALAC).

The OVCAR-3 cells were maintained in vitro as monolayer culture in RPMI 1640 medium media supplemented with 20% heat inactivated fetal bovine serum at 37° C. in an atmosphere of 5% CO₂ in air. The tumor cells were routinely sub-cultured before confluence by trypsin-EDTA treatment, not to exceed 4-5 passages. The cells growing in an exponential growth phase were harvested and counted for tumor inoculation.

Each mouse was inoculated subcutaneously on the right flank with OVCAR-3 tumor cells (2×10\hat{ }7) in 0.2 mL of 1:1 RPMI 1640 medium/matrigel for the tumor development. All animals were monitored for tumor growth. In addition, all animals were monitored for irregular behavior such as changes in mobility, food and water consumption (by cage side checking only), body weight (BW), eye/hair matting and any other abnormal effect. Body weights of all animals were measured and recorded twice weekly throughout the study. Relative body weight, expressed in %, was calculated using the following formula:

$$Relative body weight \% ( percentage of initial body weight ) = ( {BW}_{PG - DX} / {BW}_{PG - D 0} ) \times 100.$$

The measurement of tumor volume was conducted twice weekly with a caliper and recorded. The tumor volume (mm³) was estimated using the formula: TV=a×b²/2, where “a” and “b” are the length and width of the tumor, respectively.

The mice were preferentially selected for group 1 to group 6. Groups and treatments were started when the mean tumor volume reached 198 mm³. Based on the tumor volume and body weight, mice were randomly assigned to respective groups such that the average starting tumor volume and body weight were similar for each treatment group. The study groups and number of animals per group are shown in the table below. Graphical analysis of the tumor volumes over the study duration was performed with Prism 9 (GraphPad Software [Boston, MA, USA]).

### Groups and Treatments:

| | | Animals/ | Dose | Dose Vol | | | Duration |
|---|---|---|---|---|---|---|---|
| Group | Test Articles | Group | (mg/kg) | (mL/kg) | Route | Regimen | (days) |
| 1 | Vehicle1 | 6 | — | 5 | PO | BID | 21 |
| 2 | A Compound | 6 | 5 | 5 | PO | BID | 21 |
| | of Formula (I) | | | | | | |
| 3 | A Compound | 6 | 10 | 5 | PO | BID | 21 |
| | of Formula (I) | | | | | | |
| 4 | A Compound | 6 | 15 | 5 | PO | BID | 21 |
| | of Formula (I) | | | | | | |
| 5 | A Compound | 6 | 30 | 5 | PO | QD | 21 |
| | of Formula (I) | | | | | | |
| 6 | A Compound | 6 | 100 | 5 | PO | QD | 21 |
| | of Formula (I) | | | | | | |

Note:
BID interval: 12 h.

### Pharmacodynamic Evaluation in OVCAR-3 Tumor Tissue:

All the procedures related to animal handling, care, and the treatment in this study were performed according to guidelines approved by the Institutional Animal Care and Use Committee (IACUC) of Pharmaron following the guidance of the Association for Assessment and Accreditation of Laboratory Animal Care (AAALAC).

The OVCAR-3 cells were maintained in vitro as monolayer culture in RPMI 1640 medium media supplemented with 20% heat inactivated fetal bovine serum at 37° C. in an atmosphere of 5% CO₂ in air. The tumor cells were routinely sub-cultured before confluence by trypsin-EDTA treatment, not to exceed 4-5 passages. The cells growing in an exponential growth phase were harvested and counted for tumor inoculation.

Each mouse was inoculated subcutaneously on the right flank with OVCAR-3 tumor cells (2×10\hat{ }7) in 0.2 mL of 1:1 RPMI 1640 medium/matrigel for the tumor development. All animals were monitored for tumor growth. In addition, all animals were monitored for irregular behavior such as changes in mobility, food and water consumption (by cage side checking only), body weight (BW), eye/hair matting and any other abnormal effect.

Groups and treatments were started when the mean tumor volume reached 384 mm³. Based on the tumor volume and body weight, mice were randomly assigned to respective groups such that the average starting tumor volume and body weight was similar for each treatment group. The study groups and number of animals per group are shown in the table below:

### Groups and Treatments for PD Study:

| | | Animals/ | Dose | Dose Vol | | | Duration |
|---|---|---|---|---|---|---|---|
| Group | Test Articles | Group | (mg/kg) | (mL/kg) | Route | Regimen | (days) |
| 10 | Vehicle1 | 5 | — | 10 | PO | QD | 2 |
| 11 | Vehicle1 | 5 | — | 10 | PO | QD | 7 |
| 12 | A Compound | 5 | 100 | 10 | PO | QD | 1 |
| | of Formula (I) | | | | | | |
| 13 | A Compound | 5 | 100 | 10 | PO | QD | 2 |
| | of Formula (I) | | | | | | |
| 14 | A Compound | 5 | 100 | 10 | PO | QD | 7 |
| | of Formula (I) | | | | | | |
| 15 | AMG650 | 5 | 100 | 10 | PO | QD | 1 |
| 16 | AMG650 | 5 | 100 | 10 | PO | QD | 2 |
| 17 | AMG650 | 5 | 100 | 10 | PO | QD | 7 |

At the appropriate time post last dose for each group, tumors were harvested and snap frozen for PD analysis. Tumors were evaluated for Phospho-Histone H3 or Cleaved Caspase-3 (Asp175) analysis by immunohistochemistry (IHC).

### Antibody and Detection Reagent Information:

Primary Antibody: Cleaved Caspase-3(Asp175) Antibody (CS H3(Ser10) antibody (CS
Secondary Antibody: Goat anti-Rabbit IgG (H+L) Secondary Antibody, HRP (Invitrogen: 31460
Detection Reagent: DAB peroxidase (HRP) substrate kit (Vector Laboratories: SK-4100)

Antibody and Detection Reagent Information: IHC was conducted based on the standard protocol provided by antibody supplier.

Thickness of 3 um paraffin tissue sections for IHC staining was used. Sections were de-paraffin and rehydrated through xylene and gradient alcohols. A pressure cooker or microwave oven was used to repair antigen for 10-15 min with EDTA. Tissue sections were washed in PBS, 5 min 3 times each. 3% H₂O₂—PBS blocking solution for 10 min was used to block endogenous peroxidase activity. Then slides were washed in PBS for 5 min, 3 times each. Tissue sections were incubated with blocking buffer (10% normal goat serum) at room temp for 30 min, followed by incubation with the primary antibody (CC #1:400; pHH3 1:200) overnight at 4° C. Tissue sections were then washed in PBS-T buffer solution, 5 min 3 times each. Tissue sections were then incubated with secondary antibody (goat anti-rabbit 1:200) at room temp for 1 hour. Tissue sections were washed in PBS-T buffer solution for 5 min, 3 times each. Tissue sections developed the coloration with DAB substrate and color reaction was observed under a microscope. The developing time was fixed for all the sections. The color reaction was stopped with tap water. Tissue sections were counterstained for 3 min in hematoxylin and rinsed in running tap water. Tissue sections were dehydrated through gradient alcohols and xylene. Permount mounting medium was used to mount the stained sections.

All sections were stained with antibody and quantified. The entire field of each section was scanned by Panoramic MIDI. The entire field was analyzed by software ImageJ. First the total tumor area was selected, then the necrotic area was subtracted to obtain the solid tumor area. The positive expression area was selected (brown color) and used to calculate the positive area divided by the solid tumor area. Data was graphed as mean positive cell number divided by total cell number (+/−SEM) at each time point and shown graphically using Prism 9 (GraphPad Software [Boston, MA, USA]).

### Dose Dependent Efficacy and Plasma Exposure Correlation in OVCAR-3 Model:

All the procedures related to animal handling, care, and the treatment in this study were performed according to guidelines approved by the Institutional Animal Care and Use Committee (IACUC) of Pharmaron following the guidance of the Association for Assessment and Accreditation of Laboratory Animal Care (AAALAC).

The OVCAR-3 cells were maintained in vitro as monolayer culture in RPMI 1640 medium media supplemented with 20% heat inactivated fetal bovine serum at 37° C. in an atmosphere of 5% CO2 in air. The tumor cells were routinely sub-cultured before confluence by trypsin-EDTA treatment, not to exceed 4-5 passages. The cells growing in an exponential growth phase were harvested and counted for tumor inoculation.

Each mouse was inoculated subcutaneously on the right flank with OVCAR-3 tumor cells (2×10\hat{ }7) in 0.2 mL of 1:1 RPMI 1640 medium/matrigel for the tumor development. All animals were monitored for tumor growth. In addition, all animals were monitored for irregular behavior such as changes in mobility, food and water consumption (by cage side checking only), body weight (BW), eye/hair matting and any other abnormal effect. The measurement of tumor size was conducted with a caliper. The tumor volume (mm³) was estimated using the formula: TV=a×b2/2, where “a” and “b” are the length and width of the tumor, respectively. Groups and treatments were started when the mean tumor volume reached 520 mm³. Based on the tumor volume and body weight, mice were randomly assigned to respective groups such that the average starting tumor volume and body weight were similar for each treatment group. The study groups and number of animals per group are shown in the table below.

### Groups and Treatments:

| | | Animals/ | Dose | Dose Vol | | | Duration |
|---|---|---|---|---|---|---|---|
| Group | Test Article | Group | (mg/kg) | (mL/kg) | Route | Regimen | (day) |
| 1 | Vehicle1 | 4 | — | — | PO | QD | 21 |
| 2 | AMG650 | 4 | 100 | 10 | PO | QD | 21 |
| 3 | AMG650 | 4 | 30 | 10 | PO | QD | 21 |
| 4 | AMG650 | 4 | 10 | 10 | PO | QD | 21 |
| 5 | Vehicle2 | 4 | — | — | PO | BID | 21 |
| 6 | A Compound | 4 | 100 | 10 | PO | QD | 21 |
| | of Formula (I) | | | | | | |
| 7 | A Compound | 4 | 50 | 10 | PO | BID | 21 |
| | of Formula (I) | | | | | | |
| 8 | A Compound | 4 | 30 | 10 | PO | BID | 21 |
| | of Formula (I) | | | | | | |
| 9 | A Compound | 4 | 15 | 10 | PO | BID | 21 |
| | of Formula (I) | | | | | | |
| 10 | A Compound | 4 | 5 | 10 | PO | BID | 21 |
| | of Formula (I) | | | | | | |

Note:
a. BID interval: 12 h/12 h.

The amount of test article in plasma at the end of efficacy study was evaluated in bioanalytical assay by HPLC. Instrument: Shimadzu (DGU-405, Serial No: L22176153439 IX; LC-40D XS Serial No: L22426102783 AE and L22426102699 AE; SIL-40C XS, Serial No: L22456101706 AE; Plate Changer Serial No. L22226100911 CZ and L22226100907 CZ; CTO-40C: Serial No. L22246108734 IK; CBM-40: Serial No.L22116104621 CD). MS: AB API 6500+LC/MS/MS instrument (QTRAP 6500+ QTR Serial No. DZ265162312). Column: Waters XSelect HSS T3 2.5 μm (50×2.1 mm).

Plasma sample preparation: The desired serial concentrations of working solutions were achieved by diluting stock solution of analyte with 50% acetonitrile in water solution. 5 μL of working solutions (1, 2, 4, 10, 20, 100, 200, 1000, 2000, 5000 ng/mL) were added to 10 μL of the blank female BALB/c Nude Plasma to achieve calibration standards of 0.5˜2500 ng/mL (0.5, 1, 2, 5, 10, 50, 100, 500, 1000, 2500 ng/mL) in a total volume of 15 μL. Six quality control samples at 1 ng/mL, 2 ng/mL, 5 ng/mL, 50 ng/mL, 800 ng/mL and 2000 ng/mL for plasma were prepared independently of those used for the calibration curves. These QC samples were prepared on the day of analysis in the same way as calibration standards.

15 μL of standards, 15 μL of QC samples and 15 μL of unknown samples (10 μL of female BALB/c Nude plasma with 5 μL of blank solution) were added to 200 μL of acetonitrile containing IS mixture for precipitating protein respectively. Then the samples were vortexed for 30 s. After centrifugation at 4° C., 4000 rpm for 15 min. The supernatant was diluted 3 times with water. 4 μL of diluted supernatant was injected into the LC/MS/MS system for quantitative analysis.

Graphical analysis of percent tumor growth inhibition (TGI) was plotted on the left y-axis while plasma AUC (0-24 h, h*ng/mL) was plotted on the x-axis. In the left panel the total AUC is plotted, while on the right the unbound plasma concentration is plotted, calculated by multiplying the total AUC by mouse plasma protein binding value. Graphing was performed with Prism 9 (GraphPad Software [Boston, MA, USA]).

### Example A-7: Efficacy in MFM-223 Xenograft Model

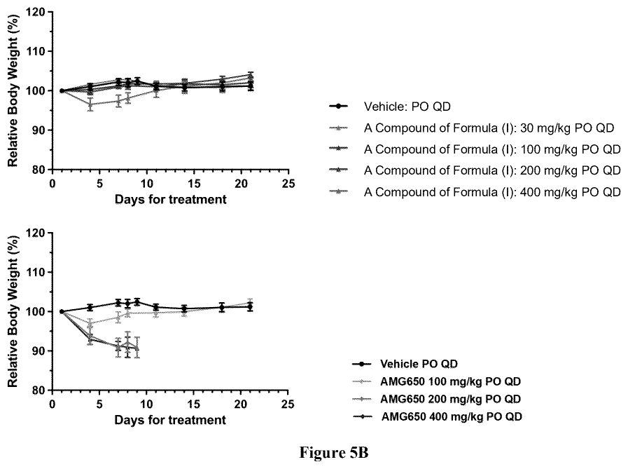

A Compound of Formula (I) exhibits a favorable therapeutic window (FIG. 5) in a MFM-223 Xenograft Model. FIG. 5A shows that treatment with A Compound of Formula (I) led to significant tumor regression at 100, 200 and 400 mg/kg dose levels in MFM-223 cells (top panel); while AMG650 treatment led to significant tumor regression at 100 mg/kg however the study had to be stopped on day 9 at dose levels >100 mg/kg (bottom panel). FIG. 5B shows that all dose levels of A Compound of Formula (I) were well tolerated throughout the treatment duration; while AMG650 was not tolerated at doses above 100 mg/kg QD.

### Methods:

### Efficacy and Body Weight Analysis of Treatment in MFM-223 Xenograft Model:

All the procedures related to animal handling, care, and the treatment in this study were performed according to guidelines approved by the Institutional Animal Care and Use Committee (IACUC) of Pharmaron following the guidance of the Association for Assessment and Accreditation of Laboratory Animal Care (AAALAC).

The MFM-223 cells were maintained in vitro as monolayer culture in MEM medium supplemented with 10% heat inactivated fetal bovine serum and 2 mM L-Glu at 37° C. in an atmosphere of 5% CO² in air. The tumor cells were routinely sub-cultured before confluence by trypsin-EDTA treatment, not to exceed 4-5 passages. The cells growing in an exponential growth phase were harvested and counted for tumor inoculation.

One day before cell inoculation, the mouse was injected estrogen (40 ug/20 uL/mouse) by s.c. After cell inoculation, the mouse was injected with estrogen twice weekly. Each mouse was inoculated subcutaneously on the right flank with 1×10\hat{ }7 cells in 0.1 ml of MEM mixed Matrigel (1:1 ratio) for tumor development.

Body weight of all animals was measured and recorded twice weekly throughout the study. Relative body weight, expressed in %, is calculated using the following formula:

$$Relative body weight ( \% ) = ( {BW}_{PG - DX} / {BW}_{PG - D 0} ) \times 100$$

Grouping and treatments started when the mean tumor volume reached about 150-200 mm³. Based on the tumor volume and body weight, mice were randomly assigned to respective groups such that the average starting tumor volume and body weight is similar for each treatment group. The study groups and number of animals per group are shown in the table below.

### Groups and Treatments:

| | | Animals/ | Dose | Dose Vol | | | Duration |
|---|---|---|---|---|---|---|---|
| Group | Test Article | Group | (mg/kg) | (μL/g) | Route | Regimen | (days) |
| 1 | Vehicle | 6 | — | 10 | PO | QD | 21 |
| 2 | A Compound | 6 | 30 | 10 | PO | QD | 21 |
| | of Formula (I) | | | | | | |
| 3 | A Compound | 6 | 100 | 10 | PO | QD | 21 |
| | of Formula (I) | | | | | | |
| 4 | A Compound | 6 | 100 | 10 | PO | BID | 21.5 |
| | of Formula (I) | | | | | | |
| 5 | A Compound | 6 | 200 | 10 | PO | QD | 21 |
| | of Formula (I) | | | | | | |
| 6 | A Compound | 6 | 200 | 10 | PO | BID | 21.5 |
| | of Formula (I) | | | | | | |
| 7 | A Compound | 6 | 400 | 10 | PO | QD | 21 |
| | of Formula (I) | | | | | | |
| 8 | AMG650 | 6 | 100 | 10 | PO | QD | 21 |
| 9 | AMG650 | 4 | 200 | 10 | PO | QD | 9 |
| 10 | AMG650 | 4 | 400 | 10 | PO | QD | 9 |

Note:
a. BID interval: 12 h

The measurement of tumor size was conducted with a caliper and recorded twice weekly throughout the study. The tumor volume (mm³) is estimated using the formula: TV=a×b²/2, where “a” and “b” are the length and width of the tumor, respectively.

The TVs are used for calculation of the tumor growth inhibition and tumor growth delay. For the tumor growth inhibition (TGI), the value using the formula:

$$\% TGI ( No Tumor Regression ) = [ 1 - ( {TreatedTV}_{final} - {TreatedTV}_{initial} ) / ( {VehicleTV}_{final} - {VehicleTV}_{initial} ) ] \times 100$$

$$\% TGI ( Tumor Regression ) = - ( {TreatedTV}_{final} - {TreatedTV}_{initial} ) / {TreatedTV}_{initial} \times 100 \% + 100 \%$$

The “TV_{final}” and “TVᵢₙᵢₜₐₗi” are the mean tumor volumes on the final day and initial day.

Graphical analysis of the tumor volumes and body weights over the study duration was performed with Prism 9 (GraphPad Software [Boston, MA, USA]).

### Example A-8: Efficacy and PK Analysis of a Compound of Formula (I) Dose Response in OVCAR-8 Xeno2Raft Model

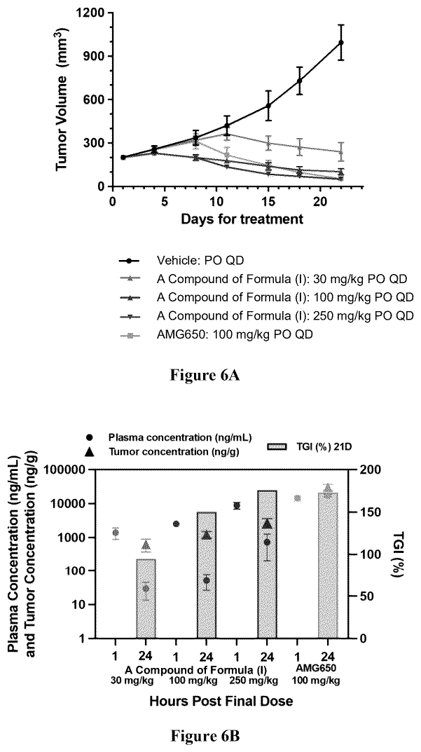

A Compound of Formula (I) achieves dose dependent tumor growth inhibition (TGI) and similar regression with lower drug concentration than AMG650 (FIG. 6). FIG. 6A shows that A Compound of Formula (I) results in dose dependent antitumor activity in a OVCAR-8 xenograft study. A Compound of Formula (I) at 100 and 250 mg/kg QD and AMG650 at 100 mg/kg QD result in similar regression in OVCAR-8 xenograft. FIG. 6B shows that plasma and tumor PK are correlated to depth of TGI response. Similar efficacy is achieved with A Compound of Formula (I) as compared to AMG650 with ˜10× less plasma and tumor exposure.

### Methods:

All the procedures related to animal handling, care, and the treatment in this study were performed according to guidelines approved by the Institutional Animal Care and Use Committee (IACUC) of Pharmaron following the guidance of the Association for Assessment and Accreditation of Laboratory Animal Care (AAALAC).

OVCAR-8 cells were maintained in vitro as a suspension culture in RPMI 1640 medium modified supplemented with 10% heat inactivated fetal bovine serum at 37° C. in an atmosphere of 5% CO₂ in air. The tumor cells were routinely sub-cultured before confluence by trypsin-EDTA treatment, not to exceed 4-5 passages. The cells growing in an exponential growth phase were harvested and counted for tumor inoculation.

Each mouse was inoculated subcutaneously on the right flank with 1×10\hat{ }7 OVCAR-8 tumor cells in 0.1 mL of mixed HC Matrigel (1:1 ratio) for tumor development.

For efficacy study, body weight of all animals was measured and recorded twice weekly throughout the study. Relative body weight, expressed in %, was calculated using the following formula:

Relative body weight % (percentage of initial body weight)=(BW_{PG-DX}/BW_{PG-D1})×100

Groups and treatments were started when the mean tumor volume reached 200 mm³. Based on the tumor volume, mice were randomly assigned to respective groups such that the average starting tumor volume was similar for each treatment group. The study groups and number of animals per group are shown in the table below.

### Groups and Treatments for Efficacy Study:

| | | Animals/ | Dose | Dose Vol | | | Duration |
|---|---|---|---|---|---|---|---|
| Group | Test Articles | Group | (mg/kg) | (mL/kg) | Route | Regimen | (days) |
| 1 | Vehicle1 | 6 | — | 10 | PO | QD | 22 |
| 2 | A Compound | 6 | 30 | 10 | PO | QD | 22 |
| | of Formula (I) | | | | | | |
| 3 | A Compound | 6 | 100 | 10 | PO | QD | 22 |
| | of Formula (I) | | | | | | |
| 4 | A Compound | 6 | 250 | 10 | PO | QD | 22 |
| | of Formula (I) | | | | | | |
| 7 | AMG650 | 6 | 100 | 10 | PO | QD | 22 |

Note:
The treatment was started on the day of mice randomization.

The measurement of tumor volume was conducted twice weekly with a caliper and recorded. The tumor volume (mm³) was estimated using the formula: TV=a×b²/2, where “a” and “b” are length and width of the tumor, respectively. The TVs were used for calculation of the tumor growth inhibition and tumor growth delay. For the tumor growth inhibition (TGI), the value using the formula:

$$\% TGI ( No Tumor Regression ) = [ 1 - ( {TreatedTV}_{final} - {TreatedTV}_{initial} ) / ( {VehicleTV}_{final} - {VehicleTV}_{initial} ) ] \times 100$$

$$\% TGI ( Tumor Regression ) = - ( {TreatedTV}_{final} - {TreatedTV}_{initial} ) / {TreatedTV}_{initial} \times 100 \% + 100 \%$$

The “TV_{final}” and “TVᵢₙᵢₜₐₗ” are the mean tumor volumes on the final day and initial day.

Graphical analysis of the tumor volumes over the study duration was performed with Prism 9 (GraphPad Software [Boston, MA, USA]).

The amount of test article in plasma and tumor tissue was evaluated in bioanalytical assay by HPLC. Instrument: Shimadzu (DGU-405, Serial No: L22176153439 IX; LC-40D XS Serial No: L22426102783 AE and L22426102699 AE; SIL-40C XS, Serial No: L22456101706 AE; Plate Changer Serial No. L22226100911 CZ and L22226100907 CZ; CTO-40C: Serial No. L22246108734 IK; CBM-40: Serial No.L22116104621 CD). MS: AB API 6500+LC/MS/MS instrument (QTRAP 6500+ QTR Serial No. DZ265162312). Column: Waters XSelect HSS T3 2.5 μm (50×2.1 mm).

Plasma sample preparation: The desired serial concentrations of working solutions were achieved by diluting stock solution of analyte with 50% acetonitrile in water solution. 5 μL of working solutions (1, 2, 4, 10, 20, 100, 200, 1000, 2000, 5000 ng/mL) were added to 10 μL of the blank female BALB/c Nude Plasma to achieve calibration standards of 0.5˜2500 ng/mL (0.5, 1, 2, 5, 10, 50, 100, 500, 1000, 2500 ng/mL) in a total volume of 15 μL. Six quality control samples at 1 ng/mL, 2 ng/mL, 5 ng/mL, 50 ng/mL, 800 ng/mLand 2000 ng/mL for plasma were prepared independently of those used for the calibration curves. These QC samples were prepared on the day of analysis in the same way as calibration standards.

15 μL of standards, 15 μL of QC samples and 15 μL of unknown samples (10 μL of female BALB/c Nude plasma with 5 μL of blank solution) were added to 200 μL of acetonitrile containing IS mixture for precipitating protein respectively. Then the samples were vortexed for 30 s. After centrifugation at 4° C., 4000 rpm for 15 min. The supernatant was diluted 3 times with water. 4 μL of diluted supernatant was injected into the LC/MS/MS system for quantitative analysis.

Tumor sample preparation: The desired serial concentrations of working solutions were achieved by diluting stock solution of analyte with 50% acetonitrile in water solution. 15 μL of working solutions (1, 2, 4, 10, 20, 100, 200, 1000, 2000, 5000 ng/mL) were added to 30 μL of the blank female BALB/c Nude tumor to achieve calibration standards of 0.5˜2500 ng/mL (0.5, 1, 2, 5, 10, 50, 100, 500, 1000, 2500 ng/mL) in a total volume of 15 μL. Six quality control samples at 1 ng/mL, 2 ng/mL, 5 ng/mL, 50 ng/mL, 800 ng/mL and 2000 ng/mL for tumor were prepared independently of those used for the calibration curves. These QC samples were prepared on the day of analysis in the same way as calibration standards.

45 μL of standards, 45 μL of QC samples and 45 μL of unknown samples (15 μL of female BALB/c Nude tumor homogenate with 30 μL of blank solution) were added to 200 μL of acetonitrile containing IS mixture for precipitating protein respectively. Then the samples were vortexed for 30 s. After centrifugation at 4° C., 4000 rpm for 15 min. The supernatant was diluted 3 times with water. 2 μL of diluted supernatant was injected into the LC/MS/MS system for quantitative analysis.

Graphical analysis of mean plasma concentration (ng/mL) and mean tumor concentration (ng/g) were plotted on the left y-axis while % TGI as calculated on day 21 was plotted on the right y-axis over the study duration. Graphing was performed with Prism 9 (GraphPad Software [Boston, MA, USA]).

### Example A-9: Antitumor Efficacy of A Compound of Formula (I) in Multiple Models

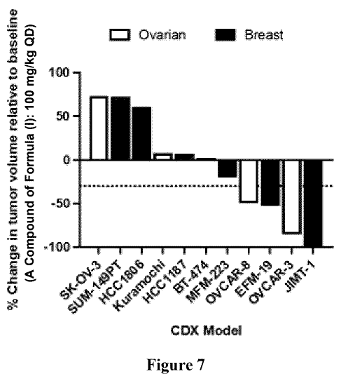

A Compound of Formula (I) achieves robust anti-tumor activity in a panel of xenograft models (FIG. 7). A Compound of Formula (I) at 100 mg/kg QD×˜21 days results in anti-tumor activity across a panel of breast and ovarian cell line derived xenograft models. A Compound of Formula (I) achieved regressions in 3/7 (43%) of breast and 2/4 (50%) of ovarian cancer models tested.

### Methods:

All xenograft models were grown as previously stated with the average tumor size at initiation of dosing to be ˜200 mm3. Animals were randomized into treatment groups for Vehicle QD PO or A Compound of Formula (I) at 100 mg/kg QD PO for ˜21 days.

The TVs were used for calculation of the tumor growth inhibition and tumor growth delay. For the tumor growth inhibition (TGI), the value using the formula:

$$\% TGI ( No Tumor Regression ) = [ 1 - ( {TreatedTV}_{final} - {TreatedTV}_{initial} ) / ( {VehicleTV}_{final} - {VehicleTV}_{initial} ) ] \times 100$$

$$\% TGI ( Tumor Regression ) = - ( {TreatedTV}_{final} - {TreatedTV}_{initial} ) / {TreatedTV}_{initial} \times 100 \% + 100 \%$$

The “TV_{final}” and “TVᵢₙᵢₜᵢₐₗ” are the mean tumor volumes on the final day and initial day.

% TGI values were graphically represented using Prism 9 (GraphPad Software [Boston, MA, USA]).

### Conclusions from Examples A-3 to A-9

A Compound of Formula (I) is a potent, selective, and differentiated KIF18A inhibitor.

A Compound of Formula (I) results in mitotic arrest and ultimately apoptosis in a cell line with high chromosomal instability (CIN), and leads to robust anti proliferation activity in a large panel of cell lines.

A Compound of Formula (I) is well tolerated and exhibits advantageous plasma and tumor levels that correlate with tumor regression, at 5-10× lower efficacious exposures than the reference molecule AMG650 (sovilnesib).

A Compound of Formula (I) demonstrates robust anti-tumor activity in a broad spectrum of xenograft models

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound having the structure of Formula (I-1): 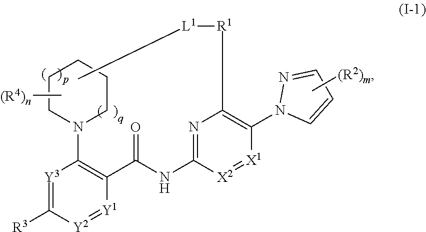
or a pharmaceutically acceptable salt solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L^{1b}-*, where * designates the attachment point to R¹;
L^{1a} is a bond, —O—, —NH—, —S—, or —SO₂—;
L^{1b} is optionally substituted C₄-C₅ alkenylene;
or L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), —CH(OR^{2a})-(optionally substituted phenyl), optionally substituted monocyclic or bicyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
each R^{2a} is independently hydrogen, optionally substituted C₁-C₆ alkyl, optionally substituted C₃-C₁₀ cycloalkyl, optionally substituted 4- to 10-membered heterocycloalkyl, or optionally substituted monocyclic heteroaryl;
or two R^{2a} on the same N atom are taken together with the N atom to which they are attached to form a 4- to 10-membered heterocycloalkyl which is optionally substituted with 1, 2, or 3 groups independently selected from halogen, —OH, oxo, —O(C₁-C₄ alkyl), —N(C₁-C₄ alkyl)₂, C₁-C₄ alkyl, C₁-C₄ haloalkyl, C₁-C₆ hydroxyalkyl —CN, —C(O)(C₁-C₆ alkyl), —S(O)₂(C₁-C₆ alkyl), and optionally substituted C₃-C₆ cycloalkyl;
each R^{2b} is independently optionally substituted C₁-C₆ alkyl, optionally substituted C₃-C₁₀ cycloalkyl, or optionally substituted 4- to 10-membered heterocycloalkyl;
each v is independently 1, 2, or 3;
R³ is —N(R^{3a})₂, —NR^{3a}(SO₂R^{3b}), —SO₂NR^{3a}R^{3b}, —SO(═NH)NR^{3a}R^{3b}, —NR^{3a}SO₂N(R^{3a})₂, —SO₂R^{3b}, —NR^{3a}C(O)—C(O)R^{3b}, —NR^{3a}C(O)OR^{3b}, or —NR^{3b}C(O)NR^{3a}R^{3a};
each R^{3a} is independently hydrogen, optionally substituted C₁-C₆ alkyl, or C₁-C₆ hydroxyalkyl;
R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl), or optionally substituted monocyclic heteroaryl;
or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
each R^{5a} is independently hydrogen or optionally substituted C₁-C₆ alkyl;
each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R⁶, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
each R^{6a} is independently hydrogen or optionally substituted C₁-C₆ alkyl;
each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4;
wherein if any L¹, L^{1b}, R², R^{2a}, R^{2b}, R³, R^{3a}, R^{3b}, R⁵, R^{5a}, R^{5b}, R⁶, R^{6a}, or R^{6b} group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl.

2. The compound of claim 1, or a pharmaceutically acceptable salt, solvate, or isotopologue thereof, wherein:
each R² is independently halogen, —CN, —(CH₂)ᵥ—CN, —OR^{2a}, —(CH₂)ᵥ—OR^{2a}, —O—(CH₂)ᵥ—R^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂)ᵥ—C(O)R^{2b}, —C(O)OR^{2a}, —(CH₂)ᵥ—C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl); or
two R² are joined together to form an optionally substituted C₃-C₁₀ cycloalkyl or an optionally substituted 4- to 10-membered heterocycloalkyl;
each R^{2a} is independently hydrogen, optionally substituted C₁-C₆ alkyl, optionally substituted C₃-C₁₀ cycloalkyl, or optionally substituted 4- to 10-membered heterocycloalkyl;
or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
each R^{2b} is independently optionally substituted C₁-C₆ alkyl;
each v is independently 1, 2, or 3; and
R³ is —N(R^{3a})₂, —NR^{3a}(SO₂R^{3b}), —SO₂NR^{3a}R^{3b}, —NR^{3a}SO₂N(R^{3a})₂, —SO₂R^{3b} or;
each R^{3a} is independently hydrogen or optionally substituted C₁-C₆ alkyl;
R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
or R^{3a} and R^{3b} are taken together with the intervening atoms to which they are attached to form an optionally substituted 4- to 8-membered heterocycloalkyl.

3. The compound of claim 1 or 2, or a pharmaceutically acceptable salt, solvate, or isotopologue thereof, wherein:
Y¹ is CR⁵;
Y² is CR⁵;
Y³ is CR⁵; and
each R⁵ is independently hydrogen, halogen, —CN, —OR^{5a}, —N(R^{5a})₂, or C₁-C₈ alkyl.

4. The compound of any one of claims 1 to 3, or a pharmaceutically acceptable salt, solvate, or isotopologue thereof, wherein:
X¹ is CR⁶ or N;
X² is CR⁶; and
each R⁶ is independently hydrogen, halogen, —CN, —OR^{5a}, —N(R^{5a})₂, or C₁-C₈ alkyl.

5. The compound of any one of claims 1 to 4, or a pharmaceutically acceptable salt, solvate, or isotopologue thereof, wherein:
p is 1.

6. The compound of any one of claims 1 to 5, or a pharmaceutically acceptable salt, solvate, or isotopologue thereof, wherein:
q is 1.

7. The compound of claim 1 or claim 2, having the structure of Formula (II): 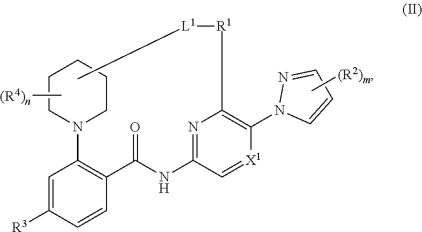
or a pharmaceutically acceptable salt, solvate, or isotopologue thereof, wherein:
X¹ is CH or N.

8. The compound of any one of claims 1 to 7, or a pharmaceutically acceptable salt, solvate, or isotopologue thereof, wherein:
R¹ is —NH— or —N(C₁-C₄ alkyl)-.

9. The compound of any one of claims 1 to 8, or a pharmaceutically acceptable salt, solvate, or isotopologue thereof, wherein:
R¹ is —NH—.

10. The compound of any one of claims 1 to 9, or a pharmaceutically acceptable salt, solvate, or isotopologue thereof, wherein:
L¹ is a linker comprising the structure -L^{1a}-L^{1b}-*, where * designates the attachment point to R¹;
L^{1a} is a bond, —O—, —NH—, —S—, or —SO₂—; and
L^{1b} is optionally substituted C₄-C₅ alkenylene.

11. The compound of claim 10, or a pharmaceutically acceptable salt, solvate, or isotopologue thereof, wherein:
L^{1a} is a bond or —O—.

12. The compound of claim 10 or 11, or a pharmaceutically acceptable salt, solvate, or isotopologue thereof, wherein:
L^{1a} is a bond.

13. The compound of claim 12, or a pharmaceutically acceptable salt, solvate, or isotopologue thereof, wherein:
L^{1b} is C₅ alkenylene.

14. The compound of claim 10 or 11, or a pharmaceutically acceptable salt, solvate, or isotopologue thereof, wherein:
L^{1a} is —O—.

15. The compound of claim 14, or a pharmaceutically acceptable salt, solvate, or isotopologue thereof, wherein:
L^{1b} is C₄ alkenylene.

16. The compound of claim 10, or a pharmaceutically acceptable salt, solvate, or isotopologue thereof, wherein:
L^{1a} is a bond; and L^{1b} is C₅ alkenylene;
or L^{1a} is —O—; and L^{1b} is C₄ alkenylene.

17. The compound of claim 10, or a pharmaceutically acceptable salt, solvate, or isotopologue thereof, wherein L¹ is —CH₂CH═CHCH₂CH₂—*, —CH₂CH₂CH═CHCH₂—*, or —OCH₂CH═CHCH₂—*, where * designates the attachment point to R¹.

18. The compound of claim 1 or claim 2, having the structure of Formula (IIIa), (IIIb), (IVa), or (IVb): 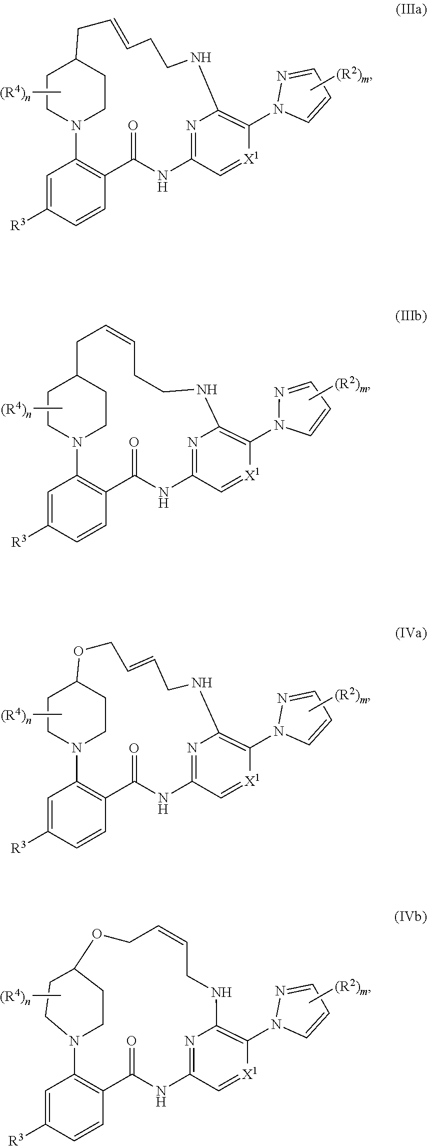
or a pharmaceutically acceptable salt, solvate, or isotopologue thereof, wherein:
X¹ is CH or N.

19. The compound of any one of claims 1 to 9, or a pharmaceutically acceptable salt, solvate, or isotopologue thereof, wherein:
L¹ is optionally substituted C₄-C₆ heteroalkylene; wherein the terminal atoms of the heteroalkylene are C, and the heteroalkylene comprises 1 or 2 non-adjacent heteroatoms independently selected from N, O, and S.

20. The compound of claim 19, or a pharmaceutically acceptable salt, solvate, or isotopologue thereof, wherein:
L¹ is optionally substituted C₄-C₅ heteroalkylene; wherein the terminal atoms of the heteroalkylene are C, and the heteroalkylene comprises 1 heteroatom selected from N, O, and S.

21. The compound of claim 19 or 20, or a pharmaceutically acceptable salt, solvate, or isotopologue thereof, wherein:
L¹ is optionally substituted C₅ heteroalkylene; wherein the terminal atoms of the heteroalkylene are C, and the heteroalkylene comprises 1 O atom.

22. The compound of claim 19, or a pharmaceutically acceptable salt, solvate, or isotopologue thereof, wherein L¹ is —CH₂OCH₂CH₂CH₂—* or —CH₂CH₂OCH₂CH₂—*, where * designates the attachment point to R¹.

23. The compound of claim 1 or claim 2, having the structure of Formula (VIa), (VIb), (VIIa), or (VIIb): 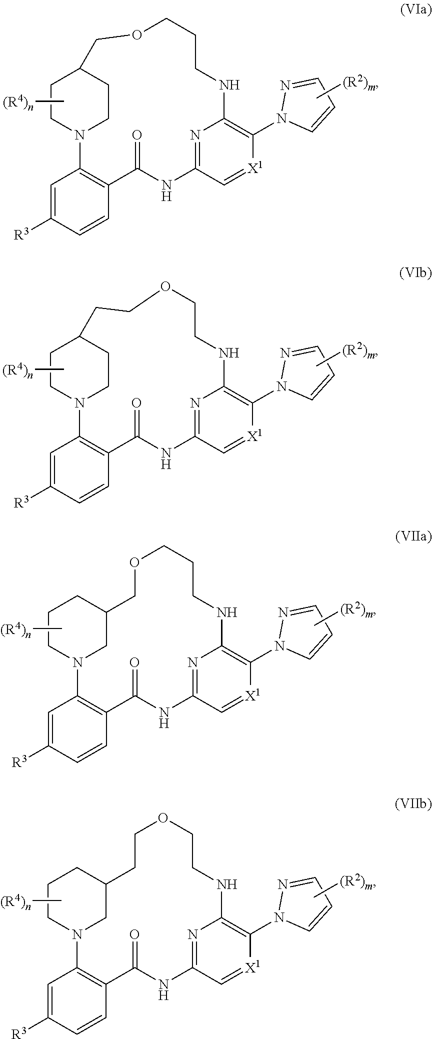
or a pharmaceutically acceptable salt, solvate, or isotopologue thereof, wherein:
X¹ is CH or N.

24. The compound of any one of claims 1 to 23, or a pharmaceutically acceptable salt, solvate, or isotopologue thereof, wherein:
X¹ is CH.

25. The compound of any one of claims 1 to 23, or a pharmaceutically acceptable salt, solvate, or isotopologue thereof, wherein:
X¹ is N.

26. The compound of any one of claims 1 or 3 to 25, or a pharmaceutically acceptable salt, solvate, or isotopologue thereof, wherein:
each R² is independently halogen, —CN, —CH₂CH₂CN, —OR^{2a}, —O—CH₂—R^{2a}, —O—(CH₂)₂—R^{2a}, —N(R^{2a})₂, —CH₂—N(R^{2a})₂, —C(O)N(R^{2a})₂, —N(R^{2a})C(O)NR^{2a}, —C(O)(C₁-C₆ alkyl), —S(O)₂N(R^{2a})₂, —S(O)₂R^{2b}, —CH₂—S(O)₂R^{2b}, C₁-C₈ alkyl, C₁-C₈ hydroxyalkyl, optionally substituted C₃-C₆ cycloalkyl, —CH₂-(optionally substituted C₃-C₆ cycloalkyl), heteroaryl, optionally substituted 4- to 8-membered heterocycloalkyl, —CH₂-(optionally substituted 4- to 10-membered heterocycloalkyl), —CH(OR^{2a})-(phenyl), optionally substituted monocyclic or bicyclic heteroaryl;
each R^{2a} is independently hydrogen, C₁-C₆ alkyl, C₃-C₆ cycloalkyl, 4- to 9-membered heterocycloalkyl, or monocyclic heteroaryl; wherein the alkyl, the C₃-C₆ cycloalkyl, and the 4- to 9-membered heterocycloalkyl is optionally substituted with 1, 2, or 3 groups independently selected from C₁-C₂ alkyl, halogen, —N(C₁-C₂ alkyl)₂, —OH, —CN, —C(O)(C₁-C₂ alkyl), —S(O)₂(C₁-C₂ alkyl), 4- to 6-membered heterocycloalkyl, C₃-C₆ cycloalkyl, and heteroaryl;
or two R^{2a} on the same N atom are taken together with the N atom to which they are attached to form a 4- to 9-membered heterocycloalkyl which is optionally substituted with 1, 2, or 3 groups independently selected from halogen, —OH, oxo, —O(C₁-C₂ alkyl), —N(C₁-C₂ alkyl)₂, C₁-C₂ alkyl, C₁-C₂ haloalkyl, C₁-C₃ hydroxyalkyl —CN, —C(O)(C₁-C₂ alkyl), —S(O)₂(C₁-C₂ alkyl), and C₃-C₆ cycloalkyl, wherein the C₃-C₆ cycloalkyl is optionally substituted with 1, 2, or 3 groups independently selected from —OH;
or two R^{2a} on the same N atom are taken together with the N atom to which they are attached to form a 4- to 6-membered heterocycloalkyl which is optionally substituted with 2 to 10 deuterium atoms;
and
each R^{2b} is independently C₁-C₆ alkyl, C₃-C₆ cycloalkyl, or 4- to 6-membered heterocycloalkyl.

27. The compound of any one of claims 1 or 3 to 26, or a pharmaceutically acceptable salt, solvate, or isotopologue thereof, wherein:
each R² is independently —F, —Cl, —Br, —CN, —CF₃, —OR^{2a}, —N(R^{2a})₂, —C(O)(C₁-C₂ alkyl), —C(O)N(R^{2a})₂, —S(O)₂R^{2b}, S(O)₂N(R^{2a})₂, C₁-C₄ hydroxyalkyl, —CH₂CF₃, —CH₂CH₂CN, —CH₂N(CH₃)₂, —CH₂S(O)₂CH₃, —CH₂NHC(O)CH₃, 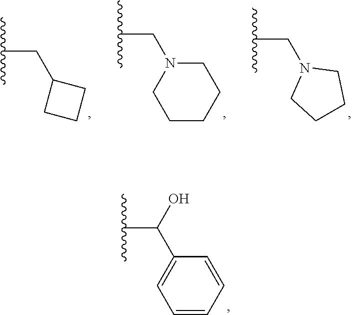 [SMILES: *CC1CCC1 ; *CN1CCCCC1 ; *CN1CCCC1 ; *C(O)c1ccccc1]
—N(R^{2a})C(O)R^{2b}, 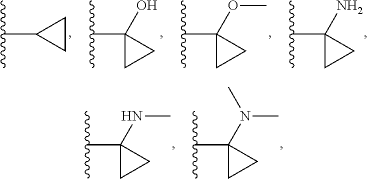
each R^{2a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CN, —CH₂CF₃, —CH₂CHF₂, —CH₂CH₂F, —CH₂CH₂OH, —CH₂CH(CH₃)OH, —CH(CH₃)CH₂OH, —C(CH₃)₂CH₂OH, 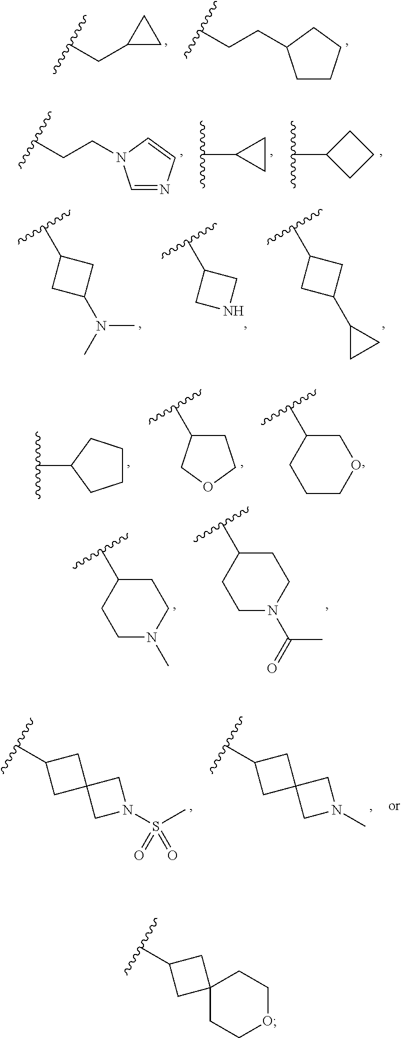
or two R^{2a} on the same N atom are taken together with the N atom to which they are attached to form a 4- to 9-membered heterocycloalkyl selected from an azetidinyl, pyrrolidinyl, piperidinyl, morpholinyl, piperazinyl, 3-oxa-8-azabicyclo[3.2.1]octanyl, 2-oxa-5-azabicyclo[2.2.1]heptanyl, oxazolidinyl, 4-oxa-7-azaspiro[2.5]octanyl, 2-oxa-6-azaspiro[3.3]heptanyl, 2-thia-6-azaspiro[3.4]octanyl, 2,7-diazaspiro[4.4]nonanyl, 2-oxa-7-azaspiro[4.4]nonanyl, 1-oxa-6-azaspiro[3.4]octanyl, 2,6-diazaspiro[3.4]octanyl, 2-thia-6-azaspiro[3.4]octanyl, thiomorpholinyl, isothiazolidinyl, and 1,4-azasilinanyl, wherein the heterocycloalkyl is optionally substituted with 1, 2, or 3 groups independently selected from —F, —OH, —OCH₃, oxo, —CH₃, —CH₂F, —CHF₂, —CF₃, —CN, —N(CH₃)₂, —S(O)₂CH₃, 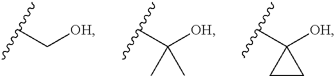
and C(O)CH₃;
or two R^{2a} on the same N atom are taken together with the N atom to which they are attached to form a 5- to 6-membered heterocycloalkyl selected from a pyrrolidinyl and a piperidinyl, wherein the pyrrolidinyl and the piperidinyl is substituted with 2-10 deuterium atoms; and
each R^{2′} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, or

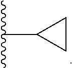 [SMILES: *C1CC1]

28. The compound of any one of claims 1 or 3 to 25, or a pharmaceutically acceptable salt, solvate, or isotopologue thereof, wherein:
each R² is independently —F, —CF₃, —Br, —Cl, —CN, —OH, —OCH₃, —C(O)CH₂CH₃, —C(O)NH₂, —C(O)NHCH₃, —C(O)N(CH₃)₂, —C(O)NH(CH₂CN), —C(O)NH(CH₂CF₃), —C(O)NH(CH₂CHF₂), —C(O)NH(CH₂CH₂OH), —S(O)₂CH₃, —CH₂CF₃, —CH₂CH₂CN, —CH₂OH, —CH₂N(CH₃)₂, —CH₂S(O)₂CH₃, —CH₂NHC(O)CH₃,

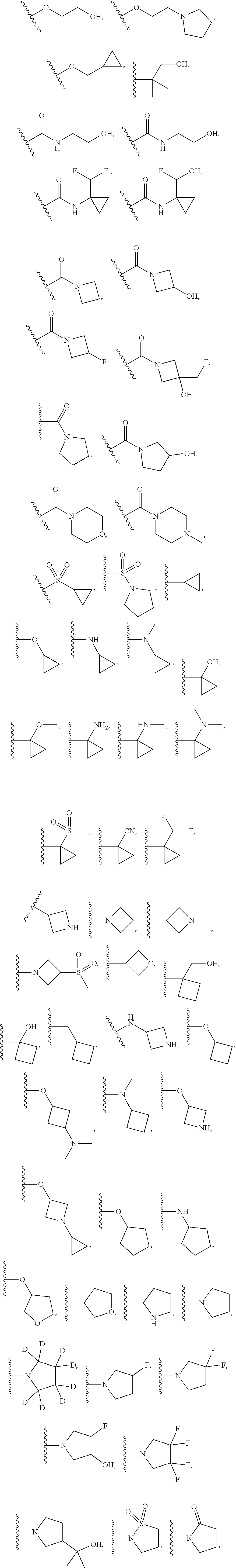

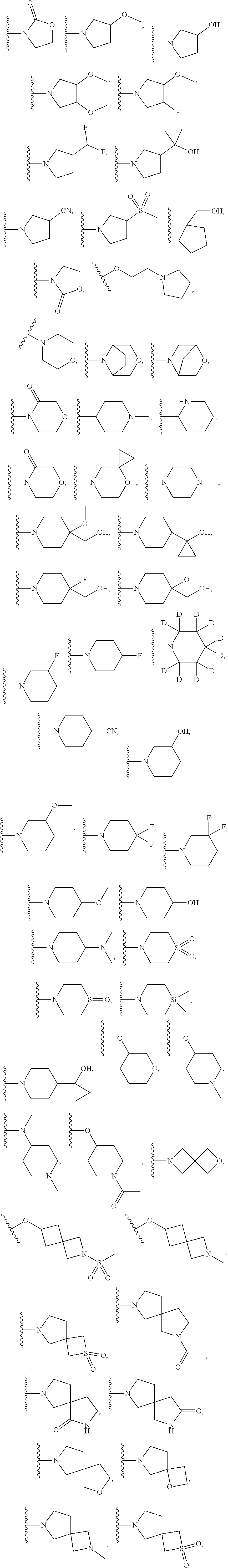

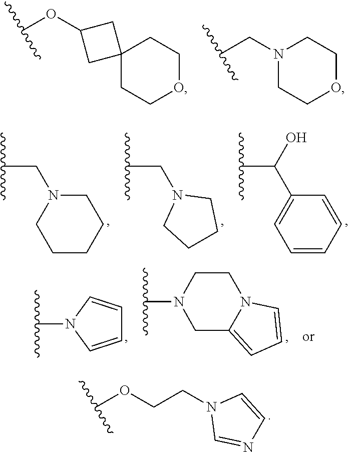

29. The compound of any one of claims 1 to 28, or a pharmaceutically acceptable salt, solvate, or isotopologue thereof, wherein:
m is 1.

30. The compound of any one of claims 1 to 29, or a pharmaceutically acceptable salt, solvate, or isotopologue thereof, wherein:
each R⁴ is independently halogen, —CN, C₁-C₆ alkyl, or C₁-C₆ fluoroalkyl.

31. The compound of any one of claims 1 to 30, or a pharmaceutically acceptable salt, solvate, or isotopologue thereof, wherein:
each R⁴ is independently —F, —Cl, —CN, —CF₃, —CHF₂, —CH₂F, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

32. The compound of any one of claims 1 to 31, or a pharmaceutically acceptable salt, solvate, or isotopologue thereof, wherein:
each R⁴ is independently —CN or —CH₃.

33. The compound of any one of claims 1 to 32, or a pharmaceutically acceptable salt, solvate, or isotopologue thereof, wherein:
n is 1.

34. The compound of any one of claims 1 or 3 to 33, or a pharmaceutically acceptable salt, solvate, or isotopologue thereof, wherein:
R³ is —N(R^{3a})₂, —NR^{3a}(SO₂R^{3b}), —SO₂NR^{3a}R^{3b}, —NR^{3a}SO(═NH)R^{3b}, —NR^{3a}SO₂N(R^{3a})₂, —SO₂R^{3b}, or —NR^{3a}C(O)—C(O)R^{3b}, —NR^{3a}C(O)OR^{3b}, or —NR^{3b}C(O)NR^{3a}R^{3a};
each R^{3a} is independently hydrogen, C₁-C₆ alkyl, or C₁-C₆ hydroxyalkyl;
R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈ cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), or optionally substituted monocyclic heteroaryl;
or R^{3a} and R^{3b} are taken together with the intervening atoms to which they are attached to form an optionally substituted 4- to 8-membered heterocycloalkyl.

35. The compound of any one of claims 1 or 3 to 34, or a pharmaceutically acceptable salt, solvate, or isotopologue thereof, wherein:
R³ is —NR^{3a}(SO₂R^{3b}), —SO₂NR^{3a}R^{3b}, —SO(═NH)NR^{3a}R^{3b}, —NR^{3a}C(O)OR^{3b}, or —NR^{3b}C(O)NR^{3a}R^{3a};
R^{3a} is hydrogen, C₁-C₆ alkyl, or hydroxyalkyl; and
R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₆ cycloalkyl, optionally substituted 4- to 6-membered heterocycloalkyl, optionally substituted (C₃-C₆ cycloalkyl)-(C₁-C₆ alkyl), optionally substituted (4- to 6-membered heterocycloalkyl)-(C₁-C₆ alkyl), or optionally substituted monocyclic 5-membered heteroaryl;
or R^{3a} and R^{3b} are taken together with the intervening atoms to which they are attached to form an optionally substituted 5- to 6-membered heterocycloalkyl.

36. The compound of claim 35, or a pharmaceutically acceptable salt, solvate, or isotopologue thereof, wherein:
R³ is —NR^{3a}(SO₂R^{3b}) or —SO₂NR^{3a}R^{3b};
R^{3a} is hydrogen, C₁-C₆ alkyl, or C₁-C₃ hydroxyalkyl; and
R^{3b} is optionally substituted C₁-C₆ alkyl, C₁-C₆ hydroxyalkyl, optionally substituted C₂-C₆ alkynyl, optionally substituted C₃-C₆ cycloalkyl, optionally substituted (C₃-C₁₀ cycloalkyl)-(C₁-C₆ alkyl), optionally substituted (4- to 10-membered heterocycloalkyl)-(C₁-C₆ alkyl), optionally substituted (heteroaryl)-(C₁-C₆ alkyl). The compound of claim 36, or a pharmaceutically acceptable salt, solvate, or isotopologue thereof, wherein:
R^{3a} is hydrogen, C₁-C₃ alkyl, or C₁-C₃ hydroxyalkyl; and
R^{3b} is optionally substituted C₁-C₃ alkyl, C₁-C₃ hydroxyalkyl, C₃-C₆ cycloalkyl which is substituted by a C₁-C₃ alkyl or C₁-C₃ hydroxyalkyl, or (C₃-C₁₀ cycloalkyl)-(C₁-C₆ alkyl) which is optionally substituted by C₁-C₃ alkyl or C₁-C₃ hydroxyalkyl. The compound of claim 35, or a pharmaceutically acceptable salt, solvate, or isotopologue thereof, wherein:
R^{3a} is hydrogen, —CH₃, —CH₂CH₃, or —CH₂CH₂OH; and
R^{3b} is —CH₃, —CHF₂, —CH₂CH₃, —CH₂CN, —CH₂CH₂CH₃, —CH(CH₃)₂, —CH₂OH, —CH₂CH₂OH, —CH₂CH₂OCH₃, —CH₂CH₂CH₂OH, —CH(CH₃)CH₂OH, —CH₂CH₂C(CH₃)₂OH, —CH₂C≡CCH₃, —CH₂CH₂C≡CH, —CH₂CH₂CH₂CH₂C≡CH,

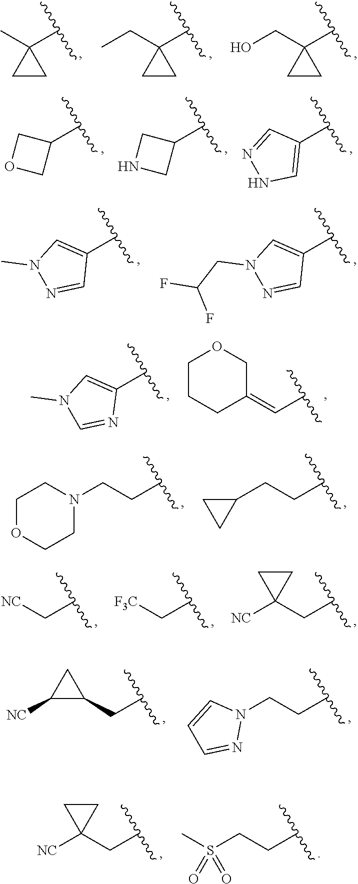

37. The compound of claim 35, or a pharmaceutically acceptable salt, solvate, or isotopologue thereof, wherein:
R^{3a} and R^{3b} are taken together with the intervening atoms to which they are attached to form an optionally substituted 5- to 6-membered heterocycloalkyl.

38. The compound of claim 35, or a pharmaceutically acceptable salt, solvate, or isotopologue thereof, wherein:
R³ is —NR^{3a}(SO₂R^{3b});
R^{3a} and R^{3b} are taken together with the intervening atoms to which they are attached to form an optionally substituted 5- to 6-membered heterocycloalkyl.

39. The compound of claim 35, or a pharmaceutically acceptable salt, solvate, or isotopologue thereof, wherein:
R³ is —NR^{3a}C(O)OR^{3b}, or —NR^{3b}C(O)NR^{3a}R^{3a};
R^{3a} and R^{3b} are taken together with the intervening atoms to which they are attached to form an optionally substituted 5- to 6-membered heterocycloalkyl.

40. The compound of claim 39, or a pharmaceutically acceptable salt, solvate, or isotopologue thereof, wherein:
R^{3a} and R^{3b} are taken together with the intervening atoms to which they are attached to form

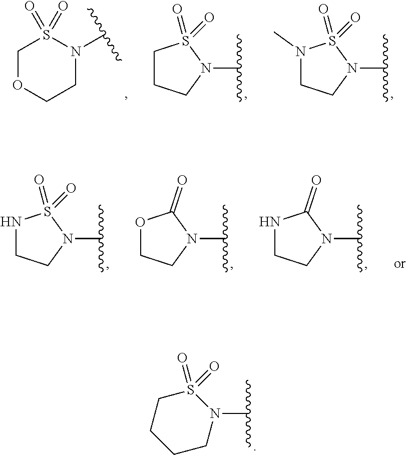

41. The compound of any one of claims 1 to 34, or a pharmaceutically acceptable salt, solvate, or isotopologue thereof, wherein:
R³ is —N(R^{3a})₂; and
each R^{3a} is independently hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

42. The compound of any one of claims 1 or 3 to 34, or a pharmaceutically acceptable salt, solvate, or isotopologue thereof, wherein:
R³ is —SO₂R^{3b}; and
R^{3b} is optionally substituted C₁-C₆ alkyl, C₁-C₆ hydroxyalkyl, optionally substituted C₂-C₆ alkenyl, C₂-C₆ alkynyl, optionally substituted C₃-C₈cycloalkyl, optionally substituted 4- to 8-membered heterocycloalkyl, optionally substituted (C₃-C₈ cycloalkyl)-(C₁-C₆ alkyl), optionally substituted (4- to 8-membered heterocycloalkyl)-(C₁-C₆ alkyl), optionally substituted monocyclic 5-membered heteroaryl, or optionally substituted (heteroaryl)-(C₁-C₆ alkyl).

43. The compound of claim 42, or a pharmaceutically acceptable salt, solvate, or isotopologue thereof, wherein:
R^{3b} is C₁-C₃ alkyl, C₁-C₃ hydroxyalkyl, C₂-C₆ alkynyl, or C₃-C₆ cycloalkyl which is substituted by a —CN, C₁-C₃ alkyl or C₁-C₃ hydroxyalkyl.

44. The compound of claim 42, or a pharmaceutically acceptable salt, solvate, or isotopologue thereof, wherein:
R^{3b} is —CH₃, —CH₂CH₃, —CH₂CH₂CH₃, —CH(CH₃)₂, —CH₂OH, —CH₂CN, —CH₂CH₂OH, —CH₂CH₂CH₂OH, —CH₂C(CH₃)₂OH, —CH₂CH₂C(CH₃)₂OH, —CH₂CH₂OCH₃, —CH₂CH₂SO₂CH₃, —CH₂CH₂OCH₂CN, —CH₂CH(CH₃)OH, —CH₂CH₂CN, —CH(CH₃)CN, —CH₂CH₂C(═CH₂)CN, —CH₂C(CH₃)(OH)CH₂OH, —CH₂C(═O)NH₂, —CH₂C≡CCH₃,

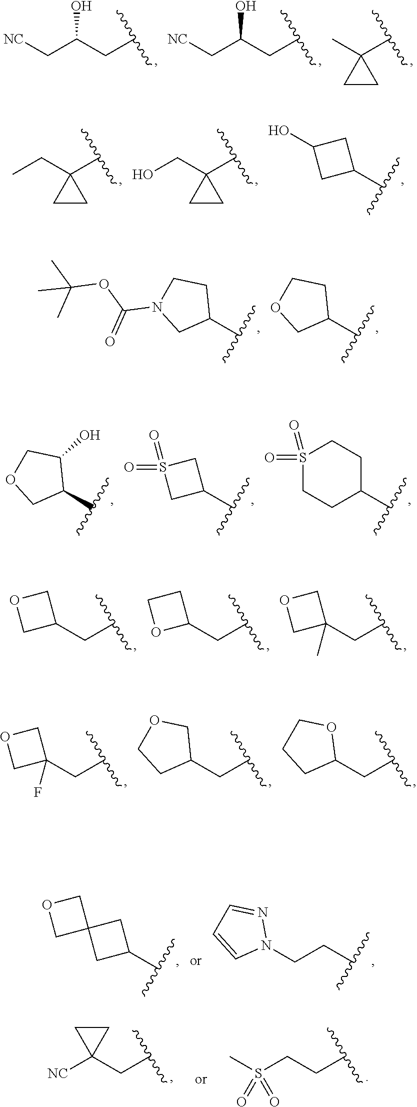

45. The compound of any one of claims 1 to 34, or a pharmaceutically acceptable salt, solvate, or isotopologue thereof, wherein:
R³ is —NR^{3a}C(O)—C(O)R^{3b};
R^{3a} is hydrogen or C₁-C₆ alkyl; and
R^{3b} is C₁-C₆ alkyl, C₁-C₆ hydroxyalkyl, or optionally substituted C₃-C₆ cycloalkyl.

46. The compound of claim 45, or a pharmaceutically acceptable salt, solvate, or isotopologue thereof, wherein:
R^{3a} is hydrogen, —CH₃, —CH₂CH₃, —CH₂CH₂CH₃, or —CH(CH₃)₂; and
R^{3b} is —CH₃, —CH₂CH₃, —CH₂CH₂CH₃, —CH(CH₃)₂, —CH₂OH, —CH₂CH₂OH, —CH₂CH₂CH₂OH,

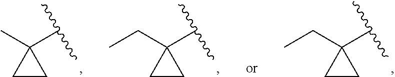

47. The compound of any one of claims 1 to 33, or a pharmaceutically acceptable salt, solvate, or isotopologue thereof, wherein:
R³ is —NH₂, —NHSO₂CH₃, —NHSO₂CHF₂, —NHSO₂CH₂CH₃, —N(CH₃)SO₂CH₃, —N(CH₃)SO₂CH₂CH₃, —NHSO₂CH₂CH₂OH, —NHSO₂CH₂CH₂OCH₃, —N(CH₃)SO₂CH₂CH₂OH, —NHSO₂CH(CH₃)CH₂OH, —NHSO₂CH₂CN, —NHSO₂CH₂CF₃, —NHSO₂CH₂CH₂C≡CH, —NHSO₂CH₂CH₂CH₂CH₂C═CH, —NHC(O)—C(O)CH₃, —NHSO₂N(CH₃)₂, —NHSO₂NH(CH₂CH₃), —SO₂CH₃, —SO₂CH₂CH₃, —SO₂CH₂CH₂OH, —SO₂CH₂CH₂OCH₃, —SO₂CH₂CH₂CH₂OH, —SO₂CH₂C(CH₃)₂OH, —SO₂CH₂CH₂C(CH₃)₂OH, —SO₂CH₂CH₂C(CH₃)₂OH, —SO₂CH(CH₃)CH₂OH, —SO₂CH₂CH₂SO₂CH₃, —SO₂CH₂CH₂OCH₂CN, —SO₂CH₂CH(CH₃)OH, —SO₂CH₂CN, —SO₂CH₂CH₂CN, —SO₂CH(CH₃)CN, —SO₂CH₂CH₂C(═CH₂)CN, —SO₂CH₂C(CH₃)(OH)CH₂OH, —SO₂CH₂C(═O)NH₂, —SO₂NHCH₃, —SO₂NHCH₂CH₃, —SO₂NHCH₂CH₂OH, —SO₂CH₂C≡CCH₃,

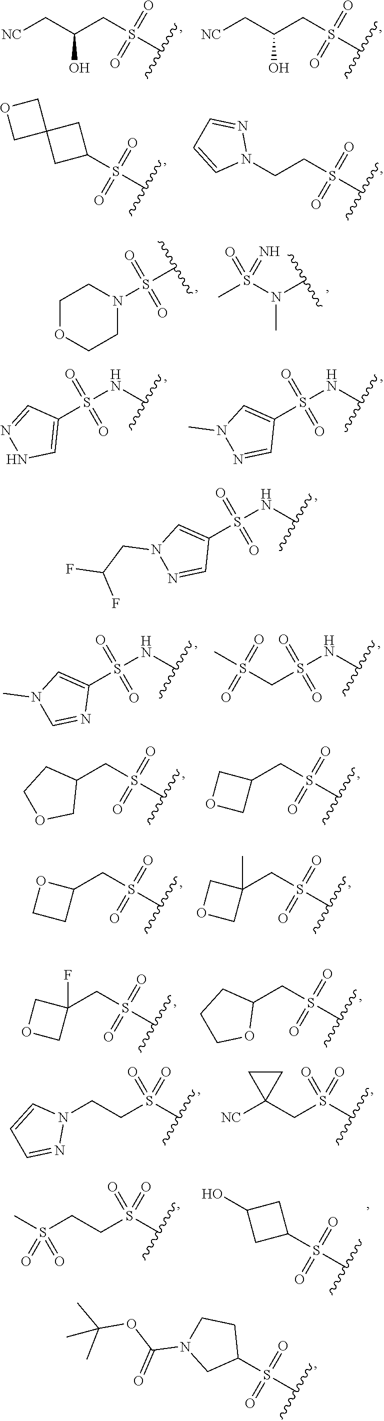

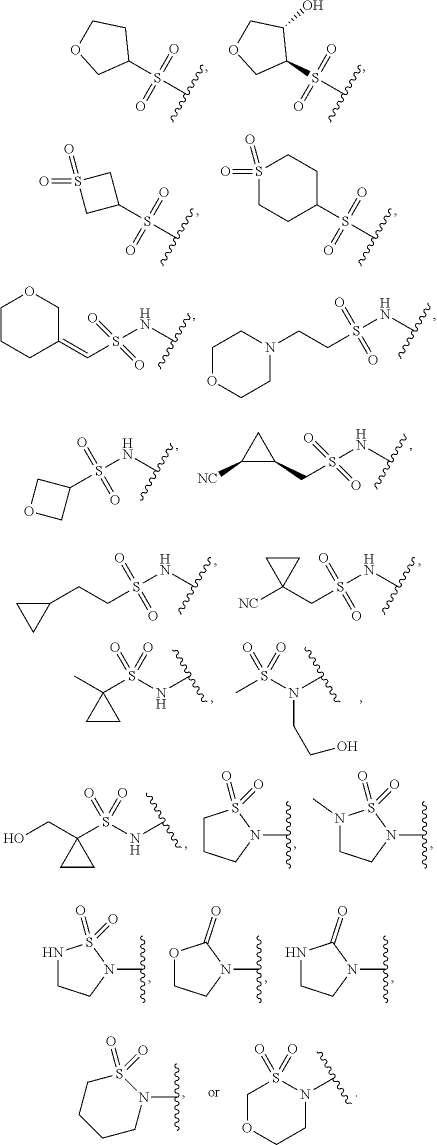

48. The compound of any one of claims 1 to 33, or a pharmaceutically acceptable salt, solvate, or isotopologue thereof, wherein:
R³ is —NHSO₂CH₂CH₃, —NHSO₂CH₂CH₂OH, —SO₂CH₂CH₃, or —SO₂CH₂CH₂OH.

49. The compound of any one of claims 1 to 33, or a pharmaceutically acceptable salt, solvate, or isotopologue thereof, wherein:
R³ is —NHSO₂CH₂CH₃ or —NHSO₂CH₂CH₂OH.

50. A compound having the structure of Formula (I): 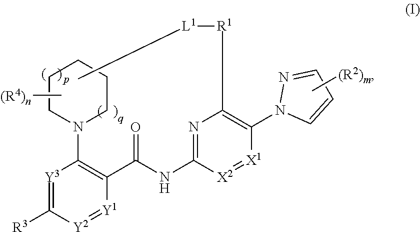
or a pharmaceutically acceptable salt, solvate, or isotopologue thereof, wherein:
Y¹, Y², and Y³ are each independently CR⁵ or N;
X¹ and X² are each independently CR⁶ or N;
L¹ is a linker comprising the structure -L^{1a}-L_{1b}-*, where * designates the attachment point to R¹;
L^{1a} is a bond, —O—, —NH—, —S—, or —SO₂—;
L^{1b} is optionally substituted C₄-C₅ alkenylene;
or LV is optionally substituted C₄-C₆ heteroalkylene; wherein the terminal atoms of the heteroalkylene are C, and the heteroalkylene comprises 1 or 2 non-adjacent heteroatoms independently selected from N, O, and S;
R¹ is a bond, —O—, —NH—, or —N(C₁-C₄ alkyl)-;
each R² is independently halogen, —CN, —(CH₂)ᵥ—CN, —OR^{2a}, —(CH₂)ᵥ—OR^{2a}, —N(R^{2a})₂, —(CH₂)ᵥ—N(R^{2a})₂, —C(O)N(R^{2a})₂, —(CH₂)ᵥ—C(O)N(R^{2a})₂, —N(R^{2a})C(O)NR^{2a}, —(CH₂)ᵥ—N(R^{2a})C(O)NR^{2a}, —C(O)R^{2b}, —(CH₂), —C(O)R^{2b}, —C(O)OR^{2a}, —(CH₂), —C(O)OR^{2a}, —S(O)₂N(R^{2a})₂, —(CH₂)ᵥ—S(O)₂N(R^{2a})₂, —S(O)₂R^{2b}, —(CH₂)ᵥ—S(O)₂R^{2b}, optionally substituted C₁-C₈ alkyl, C₁-C₈ hydroxyalkyl, optionally substituted C₃-C₁₀ cycloalkyl, —(CH₂)ᵥ-(optionally substituted C₃-C₁₀ cycloalkyl), optionally substituted 4- to 10-membered heterocycloalkyl, —(CH₂)ᵥ-(optionally substituted 4- to 10-membered heterocycloalkyl), optionally substituted phenyl, —(CH₂)ᵥ-(optionally substituted phenyl), optionally substituted monocyclic heteroaryl, or —(CH₂)ᵥ-(optionally substituted monocyclic heteroaryl);
each R^{2a} is independently hydrogen, optionally substituted C₁-C₆ alkyl, optionally substituted C₃-C₁₀ cycloalkyl, or optionally substituted 4- to 10-membered heterocycloalkyl;
or two R^{2a} on the same N atom are taken together with the N atom to which they are attached to form a 4- to 10-membered heterocycloalkyl which is optionally substituted with 1, 2, or 3 groups independently selected from halogen, —OH, C₁-C₄ alkyl, and C₁-C₄ haloalkyl;
each R^{2b} is independently optionally substituted C₁-C₆ alkyl;
each v is independently 1, 2, or 3;
R³ is —N(R^{3a})₂, —NR^{3a}(SO₂R^{3b}), —SO₂NR^{3a}R^{3b}, —NR^{3a}SO₂N(R^{3a})₂, —SO₂R^{3b}, or —NR^{3a}C(O)—C(O)R^{3b};
each R^{3a} is independently hydrogen or optionally substituted C₁-C₆ alkyl;
R^{3b} is optionally substituted C₁-C₆ alkyl, C₁-C₆ hydroxyalkyl, optionally substituted C₂-C₆ alkenyl, optionally substituted C₂-C₆ alkynyl, optionally substituted C₃-C₁₀ cycloalkyl, optionally substituted 4- to 10-membered heterocycloalkyl, optionally substituted (C₃-C₁₀ cycloalkyl)-(C₁-C₆ alkyl), optionally substituted (4- to 10-membered heterocycloalkyl)-(C₁-C₆ alkyl), or optionally substituted monocyclic heteroaryl;
or R^{3a} and R^{3b} are taken together with the intervening atoms to which they are attached to form an optionally substituted 4- to 8-membered heterocycloalkyl;
each R⁴ is independently halogen, —CN, C₁-C₆ alkyl, C₁-C₆ fluoroalkyl, C₁-C₆ aminoalkyl, C₁-C₆ alkoxy, or C₁-C₆ hydroxyalkyl;
or two R⁴ on the same carbon atom are taken together with the carbon atom to which they are attached to form a C₃-C₈ cycloalkyl;
or two R⁴ on different carbon atoms are taken together with the intervening atoms to which they are attached to form a C₁-C₃ alkylene bridge;
each R⁵ is independently hydrogen, halogen, —CN, —OR^{5a}, —N(R^{5a})₂, —C(O)N(R^{5a})₂, —N(R^{5a})C(O)NR^{5a}, —C(O)R^{5b}, —C(O)OR^{5a}, —S(O)₂N(R^{5a})₂, —S(O)₂R^{5b}, optionally substituted C₁-C₈ alkyl, optionally substituted C₃-C₈ cycloalkyl, or optionally substituted 4- to 8-membered heterocycloalkyl;
each R^{5a} is independently hydrogen or optionally substituted C₁-C₆ alkyl;
each R^{5b} is independently optionally substituted C₁-C₆ alkyl;
each R⁶ is independently hydrogen, halogen, —CN, —OR^{6a}, —N(R^{6a})₂, —C(O)N(R^{6a})₂, —N(R^{6a})C(O)NR^{6a}, —C(O)R⁶, —C(O)OR^{6a}, —S(O)₂N(R^{6a})₂, —S(O)₂R^{6b}, optionally substituted C₁-C₈ alkyl, optionally substituted C₃-C₈ cycloalkyl, or optionally substituted 4- to 8-membered heterocycloalkyl;
each R^{6a} is independently hydrogen or optionally substituted C₁-C₆ alkyl;
each R^{6b} is independently optionally substituted C₁-C₆ alkyl;
p is 0, 1, or 2;
q is 0, 1, or 2;
m is 0, 1, 2, or 3; and
n is 0, 1, 2, 3, or 4;
wherein if any L¹, L^{1b}, R², R^{2a}, R^{2b}, R³, R^{3a}, R^{3b}, R⁵, R^{5a}, R^{5b}, R⁶, R^{6a}, or Rb group is substituted then it is substituted by 1, 2, or 3 groups independently selected from halogen, oxo, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C—C₄alkyl, and —S(═O)₂C₁-C₄alkyl.

51. A compound having the structure as described in Table 1, or a pharmaceutically acceptable salt, solvate, or isotopologue thereof.

52. A pharmaceutical composition comprising a compound of any one of claims 1 to 51, or pharmaceutically acceptable salt, solvate, or isotopologue thereof, and a pharmaceutically acceptable excipient.

53. A method of treating cancer in a patient in need thereof, comprising administering to the patient a compound of any one of claims 1 to 51, or pharmaceutically acceptable salt, solvate, or isotopologue thereof.

54. The method of claim 53, wherein the cancer is bladder cancer, endometrial cancer, lung squamous cell cancer, breast cancer, colon cancer, kidney cancer, liver cancer, lung cancer, small cell lung cancer, esophagus cancer, gall-bladder cancer, brain cancer, head and neck cancer, ovarian cancer, pancreas cancer, stomach cancer, cervix cancer, thyroid cancer, prostate cancer or skin cancer.

55. A method of inhibiting KIF18A comprising contacting the enzyme with a compound of any one of claims 1 to 51, or pharmaceutically acceptable salt, solvate, or isotopologue thereof.

56. A compound of any one of claims 1 to 51, or pharmaceutically acceptable salt, solvate, or isotopologue thereof, for use in a method of treating cancer.

57. The use of claim 56, wherein the cancer is bladder cancer, endometrial cancer, lung squamous cell cancer, breast cancer, colon cancer, kidney cancer, liver cancer, lung cancer, small cell lung cancer, esophagus cancer, gall-bladder cancer, brain cancer, head and neck cancer, ovarian cancer, pancreas cancer, stomach cancer, cervix cancer, thyroid cancer, prostate cancer or skin cancer.

58. An adduct comprising a KIF18A and a compound of any one of claims 1 to 51.

59. A method of treating cancer in a patient in need thereof, comprising administering to the patient an adduct of claim 58.

60. The method of claim 59 wherein the cancer is bladder cancer, endometrial cancer, lung squamous cell cancer, breast cancer, colon cancer, kidney cancer, liver cancer, lung cancer, small cell lung cancer, esophagus cancer, gall-bladder cancer, brain cancer, head and neck cancer, ovarian cancer, pancreas cancer, stomach cancer, cervix cancer, thyroid cancer, prostate cancer or skin cancer.

61. A composition comprising a protein and a compound, wherein the protein is KIF18A, the compound is a compound of any one of claims 1 to 51, and the protein and the compound form the adduct of claim 58.
